(19) United States
(12) Patent Application Publication
(10) Pub. No.: US 2026/0114396 A1
(43) Pub. Date: Apr. 30, 2026

(54) SNP MOLECULAR MARKER ARRAY OF ALFALFA AND USE THEREOF

(71) Applicants: INSTITUTE OF ECOLOGICAL PROTECTION AND RESTORATION, CHINESE ACADEMY OF FORESTRY, Beijing (CN); HIGENTEC CO., LTD., Changsha (CN); INNER MONGOLIA GRASSLAND TECHNOLOGY INNOVATION CENTER CO., LTD, Huhhot (CN)

(72) Inventors: Xiqiang Liu, Beijing (CN); Xiaoxia Li, Beijing (CN); Youru Jiang, Changsha (CN); Shunxue Tang, Changsha (CN); Fuyang Zhou, Changsha (CN); Zhaoming Wang, Huhhot (CN); Yaling Liu, Huhhot (CN); Feng Yuan, Huhhot (CN); Weibo Ren, Huhhot (CN)

(21) Appl. No.: 19/432,429

(22) Filed: Dec. 24, 2025

Related U.S. Application Data
(63) Continuation of application No. PCT/CN2024/110703, filed on Aug. 8, 2024.

(30) Foreign Application Priority Data
Mar. 18, 2024 (CN) 202410309443.2
Jul. 9, 2024 (CN) 202410915987.3

(51) Int. Cl. A01H 1/04; C12Q 1/6827; C12Q 1/6895

(52) U.S. Cl. CPC A01H 1/045; C12Q 1/6827; C12Q 1/6895; C12Q 2600/156

(57) ABSTRACT

A SNP molecular marker array of alfalfa and the use thereof are disclosed. The SNP molecular marker array is composed of 61351 SNP molecular markers, wherein the physical positions of the 61351 SNP molecular markers are determined by sequence alignment based on a reference genome ZM-4 alfalfa genome of alfalfa. The SNP molecular markers of the present disclosure have good locus-specificity, excellent versatility, and high polymorphism and can effectively distinguish alfalfa germplasm of different origins. A genome-wide liquid-phase gene chip of alfalfa prepared using the SNP molecular markers can be used for assessment of genetic diversity in alfalfa germplasm resources, genetic map construction, functional gene mapping, genome-wide association analysis, etc., thereby providing an essential tool and technical support for molecular design breeding and verification of variety authenticity of alfalfa.

### TECHNICAL FIELD

The present disclosure relates to the field of biotechnologies, in particular to a SNP molecular marker array of alfalfa and the use thereof.

### BACKGROUND

Alfalfa (Medicago sativa L.) is one of the most important perennial leguminous forages in the world and also the earliest cultivated fine leguminous forage with the widest distribution and the highest utilization efficiency. It is known as the “King of Forage” due to its high yield, rich nutritional value, good palatability, drought resistance, and saline-alkali tolerance, as well as the characteristics of strong biological nitrogen fixation and soil fertility improvement. Alfalfa plays an irreplaceable role in promoting the adjustment of agricultural industrial structure, the development of efficient and high-quality animal husbandry, and ecological restoration.

Currently, alfalfa breeding remains at the 1.0-2.0 stage, characterized by low efficiency and accuracy in germplasm resource characterization, with severely inadequate utilization of superior germplasm resources. The lack of a high-throughput genotyping and analysis technology system, along with the absence of molecular markers with significant breeding value, has hindered the in-depth development of genome-wide selection breeding. This has resulted in low efficiency in innovation of alfalfa varieties and a scarcity of outstanding alfalfa varieties with prominent traits, severely restricting the development of the alfalfa industry. Therefore, developing a high-density genome-wide liquid-phase breeding chip of alfalfa will enable efficient and precise genotyping assay of large-scale alfalfa germplasm populations at a low cost, so as to breakthrough address bottlenecks in germplasm evaluation and variety innovation, enhance breeding efficiency, and facilitate the transition from conventional breeding to intelligent molecular design breeding for alfalfa.

### SUMMARY

The present disclosure aims at solving at least one of the above technical problems existing in the prior art. To this end, the present disclosure proposes a SNP (single nucleotide polymorphism) molecular marker array of alfalfa.

The present disclosure further proposes a primer set and/or liquid-phase probe for detecting the above SNP molecular marker array of alfalfa.

The present disclosure further proposes a gene chip having the above SNP molecular marker array of alfalfa.

The present disclosure further proposes a kit having the above SNP molecular marker array of alfalfa.

The present disclosure further proposes the application of the above SNP molecular marker array of alfalfa, gene chip, and kit.

The present disclosure further proposes a method for alfalfa breeding.

According to an aspect of the present disclosure, a SNP molecular marker array of alfalfa composed of 61351 SNP molecular markers is provided, wherein the physical positions of the 61351 SNP molecular markers are determined by sequence alignment based on the reference genome of alfalfa (ZM-4 alfalfa genome, www.figshare.com/s/fb4ba8e0b871007a9e6c), and locus information is as shown in the following Table 1.

TABLE 1
Chr: locus ref/alt

1-1: 59639T/G
1-1: 583751A/G
1-1: 612865C/A
1-1: 838619G/A
1-1: 838642C/T
1-1: 1315771G/A
1-1: 1531897T/C
1-1: 1547524T/A
1-1: 1589201C/T
1-1: 1621839C/A
1-1: 1663715A/C
1-1: 1728456T/C
1-1: 1764314A/G
1-1: 1948706T/A
1-1: 2038199G/A
1-1: 2092927C/T
1-1: 2233337T/A
1-1: 2410106A/G
1-1: 2413651T/A
1-1: 2428747G/A
1-1: 2664044T/C
1-1: 2758287C/T
1-1: 2811054G/A
1-1: 2999340T/C
1-1: 3074675A/G
1-1: 3263181A/G
1-1: 3299017G/A
1-1: 3339167T/C
1-1: 3351065C/A
1-1: 3369148A/C
1-1: 3418200G/A
1-1: 3466889C/T
1-1: 3512959G/A
1-1: 3549170T/C
1-1: 3577578G/A
1-1: 3728234G/A
1-1: 3884962A/G
1-1: 3889927A/T
1-1: 3993933T/C
1-1: 3995399G/A
1-1: 4085762C/T
1-1: 4096382C/T
1-1: 4338235A/T
1-1: 4358405A/T
1-1: 4378963G/T
1-1: 4387725C/T
1-1: 4429047T/C
1-1: 4713082T/G
1-1: 4726161A/T
1-1: 4726295G/A
1-1: 4726442C/T
1-1: 4737485G/A
1-1: 4774425A/G
1-1: 5065849G/T
1-1: 5155645A/T
1-1: 5157064A/T
1-1: 5282171G/A
1-1: 5285442G/A
1-1: 5347132T/A
1-1: 5404926T/A
1-1: 5492883T/C
1-1: 5558677C/T
1-1: 5659606A/G
1-1: 5793260C/A
1-1: 5830129C/T
1-1: 5862959C/T
1-1: 5912927A/C
1-1: 5962899A/T
1-1: 5976901C/T
1-1: 6009323C/A
1-1: 6037133G/A
1-1: 6100844C/T
1-1: 6117748C/.
1-1: 6117752G/.
1-1: 6117764T/.
1-1: 6117795T/.
1-1: 6117814A/.
1-1: 6117817A/.
1-1: 6117830A/.
1-1: 6117864A/.
1-1: 6117879A/.
1-1: 6139399G/A
1-1: 6232257C/T
1-1: 6273582T/G
1-1: 6391626T/C
1-1: 6404917C/T
1-1: 6411302A/G
1-1: 6475627C/T
1-1: 6524938G/A
1-1: 6560215A/G
1-1: 6612506T/A
1-1: 6686032T/C
1-1: 6752340C/G
1-1: 6832029A/G
1-1: 6860962G/A
1-1: 6895475T/C
1-1: 6969565C/G
1-1: 7003572C/T
1-1: 7019878T/A
1-1: 7037862C/T
1-1: 7091716G/A
1-1: 7091748G/A
1-1: 7092918A/G
1-1: 7118106T/C
1-1: 7155976T/C
1-1: 7166583G/A
1-1: 7214618A/C
1-1: 7303268A/T
1-1: 7358151C/A
1-1: 7457722T/C
1-1: 7585376A/T
1-1: 7651150C/T
1-1: 7717155G/A
1-1: 7723659C/A
1-1: 7868317T/C
1-1: 7902318G/A
1-1: 7921151C/A
1-1: 8077361A/C
1-1: 8122898A/C
1-1: 8201613G/A
1-1: 8270235C/T
1-1: 8393480T/C
1-1: 8491104T/C
1-1: 8568690T/A
1-1: 8605581G/A
1-1: 8707307C/T
1-1: 8818732C/T
1-1: 8833355C/T
1-1: 8833517T/C
1-1: 8864238C/T
1-1: 8892162A/T
1-1: 8931906C/T
1-1: 8962547C/T
1-1: 8993948A/T
1-1: 9011069C/A
1-1: 9039287G/A
1-1: 9087664A/G
1-1: 9148561G/A
1-1: 9230919A/T
1-1: 9259950A/G
1-1: 9321572G/A
1-1: 9426871T/A
1-1: 9439690A/T
1-1: 9468146A/G
1-1: 9485789T/A
1-1: 9532247A/G
1-1: 9589131T/C
1-1: 9694327G/T
1-1: 9775916T/C
1-1: 9832669C/T
1-1: 9886900G/A
1-1: 9935801C/A
1-1: 9986970T/C
1-1: 10046328A/G
1-1: 10056669T/G
1-1: 10107045A/C
1-1: 10184863T/C
1-1: 10221656G/A
1-1: 10235617T/G
1-1: 10322368C/T
1-1: 10385681A/G
1-1: 10406019A/C
1-1: 10406056G/A
1-1: 10422928C/T
1-1: 10485915A/T
1-1: 10581458A/G
1-1: 10637452C/T
1-1: 10645370C/G
1-1: 10645475A/T
1-1: 10647983A/T
1-1: 10658843A/G
1-1: 10699208T/C
1-1: 10712743T/C
1-1: 10777646T/C
1-1: 10825042T/A
1-1: 10832191A/C
1-1: 10839212T/A
1-1: 10854870A/G
1-1: 10953889C/T
1-1: 11006287G/T
1-1: 11054278A/T
1-1: 11065066A/T
1-1: 11068227C/T
1-1: 11078739C/T
1-1: 11090193G/A
1-1: 11101656A/G
1-1: 11127600T/C
1-1: 11144803T/A
1-1: 11154895A/C
1-1: 11176449G/A
1-1: 11203377T/C
1-1: 11214947A/G
1-1: 11222057G/T
1-1: 11222066T/C
1-1: 11229916C/A
1-1: 11232719A/G
1-1: 11257796A/C
1-1: 11289338C/G
1-1: 11338419G/A
1-1: 11637642A/G
1-1: 11655537C/T
1-1: 11734527A/G
1-1: 11779694A/G
1-1: 11781516T/C
1-1: 11845143A/G
1-1: 11871813G/A
1-1: 11922231T/A
1-1: 11924343T/C
1-1: 11945234T/A
1-1: 11977246A/G
1-1: 12009573C/T
1-1: 12089849A/G
1-1: 12172968G/A
1-1: 12210123A/G
1-1: 12354815A/T
1-1: 12379270T/G
1-1: 12540554C/T
1-1: 12590652A/G
1-1: 12608587G/A
1-1: 12631852T/C
1-1: 12684339G/A
1-1: 12748944G/A
1-1: 12893788A/G
1-1: 12931697A/T
1-1: 13013688T/C
1-1: 13049687C/G
1-1: 13141192T/C
1-1: 13222538C/T
1-1: 13312085C/T
1-1: 13395766A/G
1-1: 13428437C/T
1-1: 13441082A/C
1-1: 13463117A/G
1-1: 13511897T/C
1-1: 13516958C/T
1-1: 13549952T/G
1-1: 13575837C/T
1-1: 13613593G/C
1-1: 13720353T/C
1-1: 13721942A/T
1-1: 13753929C/T
1-1: 13777256G/A
1-1: 13806073A/T
1-1: 13807154A/T
1-1: 13826099A/T
1-1: 13837208C/G
1-1: 13951825C/A
1-1: 14148037T/A
1-1: 14257032G/A
1-1: 14257597G/A
1-1: 14278841G/T
1-1: 14289958T/G
1-1: 14298464C/T
1-1: 14312150C/T
1-1: 14313102T/C
1-1: 14316285G/T
1-1: 14416115A/C
1-1: 14416799A/G
1-1: 14426764T/C
1-1: 14429176G/C
1-1: 14492353T/G
1-1: 14531268G/A
1-1: 14595933T/G
1-1: 14614386A/G
1-1: 14636069A/T
1-1: 14699986C/T
1-1: 14786554G/A
1-1: 14800002G/A
1-1: 14814136A/T
1-1: 14857658C/T
1-1: 14859587A/T
1-1: 14873339A/G
1-1: 14887197A/G
1-1: 14898404C/G
1-1: 14982742G/A
1-1: 15065864G/A
1-1: 15120775A/G
1-1: 15185677G/A
1-1: 15208061C/T
1-1: 15252678C/T
1-1: 15457227C/T
1-1: 15538542G/A
1-1: 15540194C/T
1-1: 15637781A/G
1-1: 15736759T/C
1-1: 15780793A/G
1-1: 15937882A/T
1-1: 15939792G/A
1-1: 15952763C/T
1-1: 15970892C/T
1-1: 16080092G/A
1-1: 16140946G/T
1-1: 16155655C/T
1-1: 16247186C/G
1-1: 16265764G/T
1-1: 16276018G/A
1-1: 16287310G/A
1-1: 16328252G/C
1-1: 16608820T/A
1-1: 16613317C/A
1-1: 16641215G/A
1-1: 16685541C/A
1-1: 16757475T/C
1-1: 16769836C/T
1-1: 16788990T/C
1-1: 16808066T/C
1-1: 16820152G/A
1-1: 16821049G/T
1-1: 16821178T/A
1-1: 16821268G/A
1-1: 16821335G/A
1-1: 16838549C/T
1-1: 16858843T/C
1-1: 16859295G/A
1-1: 16881651T/G
1-1: 16910745T/C
1-1: 16995192A/G
1-1: 16995300G/A
1-1: 17185552C/G
1-1: 17232271G/A
1-1: 17274780T/G
1-1: 17293817A/G
1-1: 17331814G/A
1-1: 17388185T/C
1-1: 17419680T/C
1-1: 17444891T/C
1-1: 17448338A/G
1-1: 17448350G/T
1-1: 17484165G/A
1-1: 17495248T/A
1-1: 17533926A/C
1-1: 17584562G/A
1-1: 17590094C/T
1-1: 17590287T/A
1-1: 17630869G/T
1-1: 17688180A/G
1-1: 17745815A/G
1-1: 17774966C/T
1-1: 17815450A/C
1-1: 17858231C/T
1-1: 17906711C/A
1-1: 18004758G/A
1-1: 18020206A/T
1-1: 18056212A/G
1-1: 18121215G/T
1-1: 18132688C/G
1-1: 18153863C/T
1-1: 18165612C/A
1-1: 18214664T/C
1-1: 18241040T/A
1-1: 18271255A/G
1-1: 18323922G/A
1-1: 18324444G/T
1-1: 18355043T/A
1-1: 18381686T/C
1-1: 18392208A/G
1-1: 18404347T/G
1-1: 18487692T/G
1-1: 18520728C/G
1-1: 18571222C/T
1-1: 18600523T/G
1-1: 18641650G/A
1-1: 18682741A/G
1-1: 18743838T/C
1-1: 18824036T/C
1-1: 18952065A/G
1-1: 18983436A/T
1-1: 18997478A/T
1-1: 19050473G/A
1-1: 19114378G/A
1-1: 19148119A/G
1-1: 19194845C/T
1-1: 19377794C/A
1-1: 19410183C/T
1-1: 19507576C/T
1-1: 19547453C/T
1-1: 19580016A/C
1-1: 19715911C/T
1-1: 19802561T/C
1-1: 19871768C/G
1-1: 19993650A/G
1-1: 19997210A/G
1-1: 20145377C/T
1-1: 20180516T/C
1-1: 20248441G/T
1-1: 20258712C/G
1-1: 20282937G/A
1-1: 20299323T/C
1-1: 20303028G/A
1-1: 20303381T/C
1-1: 20303486G/A
1-1: 20304142T/A
1-1: 20319397A/G
1-1: 20345863C/G
1-1: 20369759C/T
1-1: 20390293G/C
1-1: 20406316G/A
1-1: 20410084G/C
1-1: 20410522T/A
1-1: 20410945G/A
1-1: 20461745A/C
1-1: 20513846T/A
1-1: 20532915C/T
1-1: 20555638G/A
1-1: 20558079T/C
1-1: 20558442A/G
1-1: 20558500G/A
1-1: 20558606G/A
1-1: 20558723A/G
1-1: 20571464C/A
1-1: 20571607C/T
1-1: 20571608G/A
1-1: 20583583T/C
1-1: 20597349C/A
1-1: 20632950C/T
1-1: 20633507A/C
1-1: 20644397C/T
1-1: 20644704G/A
1-1: 20655920G/A
1-1: 20672129A/G
1-1: 20672175T/G
1-1: 20685445G/A
1-1: 20685506T/A
1-1: 20723831T/A
1-1: 20761782G/A
1-1: 20761867T/C
1-1: 20791470T/C
1-1: 20817939T/C
1-1: 20843074T/A
1-1: 20870543G/T
1-1: 20890242C/T
1-1: 20924255C/T
1-1: 21017282T/A
1-1: 21046013C/T
1-1: 21072173A/G
1-1: 21103434G/A
1-1: 21210059T/G
1-1: 21240544C/G
1-1: 21240951A/C
1-1: 21253640T/C
1-1: 21279765G/A
1-1: 21281852G/A
1-1: 21281882T/C
1-1: 21282059A/C
1-1: 21282069C/A
1-1: 21312555G/A
1-1: 21317809G/T
1-1: 21317812T/.
1-1: 21317815T/.
1-1: 21352451G/A
1-1: 21410141A/G
1-1: 21533494T/C
1-1: 21553654G/A
1-1: 21554006T/A
1-1: 21580032G/T
1-1: 21596082A/G
1-1: 21613146G/A
1-1: 21628622C/A
1-1: 21633760T/A
1-1: 21643862T/G
1-1: 21700636T/C
1-1: 21708406T/C
1-1: 21874049G/T
1-1: 21905070C/T
1-1: 21929897A/T
1-1: 21929899A/T
1-1: 22019220T/C
1-1: 22112652G/A
1-1: 22184920A/C
1-1: 22303434T/C
1-1: 22303438C/A
1-1: 22303451C/G
1-1: 22442561T/C
1-1: 22451414T/A
1-1: 22475918G/A
1-1: 22488523C/T
1-1: 22502727C/T
1-1: 22513261C/T
1-1: 22518116G/T
1-1: 22518528A/G
1-1: 22518536T/C
1-1: 22523842G/T
1-1: 22524804A/T
1-1: 22527390A/C
1-1: 22527412G/A
1-1: 22528568T/C
1-1: 22529249T/C
1-1: 22529287G/A
1-1: 22529426C/T
1-1: 22529924C/G
1-1: 22530961T/A
1-1: 22531073G/A
1-1: 22538877A/T
1-1: 22563417C/T
1-1: 22576704T/C
1-1: 22580735T/G
1-1: 22580993T/A
1-1: 22596276T/A
1-1: 22596301A/G
1-1: 22631636T/C
1-1: 22647004G/A
1-1: 22666868T/C
1-1: 22697260A/C
1-1: 22816845C/G
1-1: 22863909T/C
1-1: 22885001A/T
1-1: 22910230C/T
1-1: 22942971C/G
1-1: 22970642C/T
1-1: 23103875T/G
1-1: 23103977C/T
1-1: 23116945A/T
1-1: 23123918G/T
1-1: 23129841T/C
1-1: 23136177C/A
1-1: 23202336G/A
1-1: 23216514C/T
1-1: 23241634A/G
1-1: 23279825A/C
1-1: 23350625G/A
1-1: 23366381T/C
1-1: 23394704C/T
1-1: 23503079T/C
1-1: 23513010C/T
1-1: 23692416G/T
1-1: 23715554A/T
1-1: 23717864G/A
1-1: 23737998A/G
1-1: 23794732T/C
1-1: 23908217C/A
1-1: 23912226A/C
1-1: 23935633C/T
1-1: 23948164T/C
1-1: 23962060A/T
1-1: 23965971C/A
1-1: 23982776A/C
1-1: 24027368C/T
1-1: 24109956T/C
1-1: 24180868A/G
1-1: 24341893G/C
1-1: 24352213T/A
1-1: 24387411G/A
1-1: 24397597T/A
1-1: 24503426A/C
1-1: 24538027G/A
1-1: 24538696T/C
1-1: 24538768A/G
1-1: 24541053T/C
1-1: 24621670G/A
1-1: 24647586T/C
1-1: 24666817A/C
1-1: 24682116G/A
1-1: 24682447G/A
1-1: 24682467T/A
1-1: 24682480A/T
1-1: 24751490T/C
1-1: 24785090T/C
1-1: 24886910G/T
1-1: 24889590T/C
1-1: 24983380C/T
1-1: 25055171T/G
1-1: 25065352T/C
1-1: 25144203C/A
1-1: 25188475A/G
1-1: 25229996A/T
1-1: 25232398T/C
1-1: 25245771A/G
1-1: 25261764A/G
1-1: 25278928G/T
1-1: 25296212G/T
1-1: 25306313T/A
1-1: 25315035G/A
1-1: 25596496C/T
1-1: 25731042T/A
1-1: 25769880G/A
1-1: 25858683A/C
1-1: 25872436A/T
1-1: 25919595T/C
1-1: 25930001C/A
1-1: 25942290A/T
1-1: 25980139C/T
1-1: 25993959A/G
1-1: 26034444A/T
1-1: 26060773A/T
1-1: 26091197A/G
1-1: 26092365A/T
1-1: 26110298C/T
1-1: 26121365A/G
1-1: 26122221G/A
1-1: 26122289A/G
1-1: 26122321G/A
1-1: 26122353C/T
1-1: 26122776C/T
1-1: 26133223T/A
1-1: 26133411A/T
1-1: 26145911C/A
1-1: 26164579T/C
1-1: 26166274T/A
1-1: 26166278C/A
1-1: 26166403T/C
1-1: 26166513T/C
1-1: 26175015G/A
1-1: 26228518C/T
1-1: 26268024T/C
1-1: 26385462G/A
1-1: 26437596T/A
1-1: 26481120C/A
1-1: 26502661G/A
1-1: 26571187G/A
1-1: 26573449G/A
1-1: 26573515A/G
1-1: 26592473T/C
1-1: 26602911A/C
1-1: 26607621T/G
1-1: 26608044T/C
1-1: 26628224A/G
1-1: 26684900T/C
1-1: 26731540T/C
1-1: 26753018A/T
1-1: 26895407C/T
1-1: 26964916C/A
1-1: 27063083T/C
1-1: 27127534C/A
1-1: 27210044G/A
1-1: 27297499A/G
1-1: 27343424G/T
1-1: 27375107G/A
1-1: 27484480A/T
1-1: 27497123G/T
1-1: 27555840A/G
1-1: 27628657C/G
1-1: 27668509T/A
1-1: 27724151C/T
1-1: 27777837C/T
1-1: 27889766C/A
1-1: 27932975C/T
1-1: 27945684C/T
1-1: 27950886T/C
1-1: 27963033T/C
1-1: 28045406C/T
1-1: 28115387A/T
1-1: 28132405T/C
1-1: 28164782C/T
1-1: 28165040T/C
1-1: 28165167A/G
1-1: 28184978A/G
1-1: 28233351G/A
1-1: 28305424C/T
1-1: 28345023A/G
1-1: 28356353C/A
1-1: 28375173A/G
1-1: 28379037A/C
1-1: 28385110G/A
1-1: 28409253C/A
1-1: 28489477C/T
1-1: 28528060T/G
1-1: 28562684G/A
1-1: 28600264C/T
1-1: 28626843C/T
1-1: 28678221G/A
1-1: 28709357G/A
1-1: 28739906A/G
1-1: 28828046C/T
1-1: 28865290A/T
1-1: 28876027A/T
1-1: 28887831T/A
1-1: 28892639A/G
1-1: 28898190G/A
1-1: 28918112G/A
1-1: 28959495C/T
1-1: 28977842T/C
1-1: 29248146G/T
1-1: 29317904G/A
1-1: 29405973G/A
1-1: 29453716A/C
1-1: 29466819T/A
1-1: 29575333G/A
1-1: 29607526A/G
1-1: 29651320T/C
1-1: 29706507T/C
1-1: 29792241C/T
1-1: 29916667G/T
1-1: 29941989G/A
1-1: 30039580A/T
1-1: 30054568C/T
1-1: 30056634T/A
1-1: 30064611T/C
1-1: 30109384T/G
1-1: 30127733T/C
1-1: 30213994A/G
1-1: 30258270G/A
1-1: 30288507T/C
1-1: 30383433G/A
1-1: 30418113T/C
1-1: 30459351A/G
1-1: 30505045C/T
1-1: 30523631G/A
1-1: 30593842T/G
1-1: 30692096A/C
1-1: 30803783G/A
1-1: 30815801C/T
1-1: 30839818C/T
1-1: 30840402G/A
1-1: 30880834C/T
1-1: 30982682T/A
1-1: 30985798G/T
1-1: 31038354A/G
1-1: 31124260G/A
1-1: 31139689A/T
1-1: 31153736T/C
1-1: 31172553T/C
1-1: 31177912A/G
1-1: 31194597A/G
1-1: 31282512C/T
1-1: 31304703A/G
1-1: 31333661G/A
1-1: 31371900T/A
1-1: 31473830G/A
1-1: 31475031A/T
1-1: 31486052A/T
1-1: 31655809C/T
1-1: 31750435T/A
1-1: 31781993G/C
1-1: 31841258C/A
1-1: 31901942C/T
1-1: 31976703T/C
1-1: 32041533C/T
1-1: 32042064G/A
1-1: 32042131G/A
1-1: 32055807C/T
1-1: 32170387T/C
1-1: 32367309G/A
1-1: 32378357T/A
1-1: 32454127G/A
1-1: 32457781A/G
1-1: 32499511T/C
1-1: 32511000C/T
1-1: 32604250C/T
1-1: 32619028A/T
1-1: 32747797C/T
1-1: 32760249A/G
1-1: 32826216T/C
1-1: 32885254T/G
1-1: 32938996C/T
1-1: 32989283G/C
1-1: 33094810A/G
1-1: 33172183T/G
1-1: 33244872T/C
1-1: 33256592C/G
1-1: 33302944T/C
1-1: 33330510T/G
1-1: 33356095A/G
1-1: 33356573G/A
1-1: 33357874G/A
1-1: 33401860T/A
1-1: 33512710C/T
1-1: 33541675C/T
1-1: 33627256C/A
1-1: 33688826A/G
1-1: 33703069T/A
1-1: 33713411A/G
1-1: 33748599T/A
1-1: 33778764G/A
1-1: 33804933A/T
1-1: 33921314A/T
1-1: 33951635C/A
1-1: 33998753G/A
1-1: 34042538G/A
1-1: 34042675G/T
1-1: 34044463A/T
1-1: 34044629A/T
1-1: 34044675G/A
1-1: 34069192A/G
1-1: 34095350T/C
1-1: 34153488G/A
1-1: 34164718T/C
1-1: 34176159T/C
1-1: 34189379A/T
1-1: 34194339C/T
1-1: 34194495C/T
1-1: 34194743C/T
1-1: 34194978C/A
1-1: 34195079C/T
1-1: 34195238A/G
1-1: 34195344C/T
1-1: 34195407T/C
1-1: 34195877C/T
1-1: 34196017T/C
1-1: 34196438A/G
1-1: 34234452C/T
1-1: 34276946C/G
1-1: 34322586T/C
1-1: 34356331G/A
1-1: 34403921G/A
1-1: 34429760G/T
1-1: 34497766A/G
1-1: 34527519G/A
1-1: 34558162G/C
1-1: 34611562G/A
1-1: 34669597G/T
1-1: 34696959A/T
1-1: 34701208T/C
1-1: 34724955C/A
1-1: 34724974T/G
1-1: 34795578G/T
1-1: 34795804C/T
1-1: 34795929T/C
1-1: 34796224C/T
1-1: 34796326T/C
1-1: 34796561A/G
1-1: 34796588T/C
1-1: 34797190G/A
1-1: 34832096G/C
1-1: 34845290C/T
1-1: 34860975T/C
1-1: 34862566A/G
1-1: 34862668T/C
1-1: 34865429G/A
1-1: 34875460G/T
1-1: 34914985A/G
1-1: 34953172T/C
1-1: 35032108G/A
1-1: 35059468C/T
1-1: 35071707C/T
1-1: 35120446G/A
1-1: 35122540G/A
1-1: 35144932G/T
1-1: 35195550C/T
1-1: 35226100T/C
1-1: 35284734G/A
1-1: 35375091C/A
1-1: 35400291G/A
1-1: 35436606T/A
1-1: 35472382A/C
1-1: 35503258C/T
1-1: 35550854G/C
1-1: 35551212G/A
1-1: 35598944T/A
1-1: 35610218G/A
1-1: 35622556C/A
1-1: 35656796G/T
1-1: 35701340C/G
1-1: 35747149A/G
1-1: 35783966A/C
1-1: 35827701C/G
1-1: 35914816T/C
1-1: 36117592A/G
1-1: 36120889C/T
1-1: 36137856C/T
1-1: 36264071C/T
1-1: 36490808A/G
1-1: 36505512G/A
1-1: 36624367G/A
1-1: 36637717G/T
1-1: 36678974T/C
1-1: 36680008T/C
1-1: 36693303C/A
1-1: 36724667C/G
1-1: 36753023A/G
1-1: 36762613G/A
1-1: 36777220C/T
1-1: 36837368T/C
1-1: 36874953C/T
1-1: 36941462A/C
1-1: 37026122A/C
1-1: 37154460A/C
1-1: 37170680G/T
1-1: 37173124A/C
1-1: 37208546G/C
1-1: 37326369C/T
1-1: 37369538A/G
1-1: 37559760A/G
1-1: 37667505G/A
1-1: 37741690C/A
1-1: 37777737G/C
1-1: 38000409A/G
1-1: 38057365T/G
1-1: 38091306A/T
1-1: 38135636C/G
1-1: 38194767G/A
1-1: 38312394C/G
1-1: 38332976G/A
1-1: 38377911G/T
1-1: 38445574A/T
1-1: 38513983C/A
1-1: 38803542A/G
1-1: 38845158A/C
1-1: 38845985C/T
1-1: 38874986A/T
1-1: 38894187A/C
1-1: 38924138A/G
1-1: 38935762G/A
1-1: 38994013G/T
1-1: 39007227A/G
1-1: 39009271T/C
1-1: 39028623C/T
1-1: 39030473G/A
1-1: 39086419T/A
1-1: 39301009C/T
1-1: 39379889G/T
1-1: 39409205G/A
1-1: 39478475C/A
1-1: 39569249A/T
1-1: 39632566A/G
1-1: 39642630G/A
1-1: 39785082G/T
1-1: 39867586T/A
1-1: 39944871G/A
1-1: 39950903C/T
1-1: 39976386T/G
1-1: 40025997G/A
1-1: 40115480A/G
1-1: 40118777A/G
1-1: 40119211T/A
1-1: 40120591T/C
1-1: 40180459A/G
1-1: 40250217G/A
1-1: 40262029G/A
1-1: 40287426T/C
1-1: 40321634A/T
1-1: 40324881G/A
1-1: 40325163A/G
1-1: 40325283T/A
1-1: 40325933G/T
1-1: 40327489C/T
1-1: 40327677A/T
1-1: 40333628A/C
1-1: 40366150C/T
1-1: 40383807A/G
1-1: 40388095T/C
1-1: 40460877G/A
1-1: 40486284G/A
1-1: 40505661G/A
1-1: 40532253C/T
1-1: 40566550C/T
1-1: 40592296A/G
1-1: 40616150C/T
1-1: 40630585C/A
1-1: 40659292C/T
1-1: 40668497A/G
1-1: 40668643C/T
1-1: 40685248A/G
1-1: 40711458T/C
1-1: 40873821C/T
1-1: 40884626C/T
1-1: 40886858C/T
1-1: 40912757A/G
1-1: 40950046T/A
1-1: 41039959C/A
1-1: 41097959A/G
1-1: 41130818C/T
1-1: 41149597T/A
1-1: 41184658C/T
1-1: 41242192C/T
1-1: 41246041A/G
1-1: 41263610G/T
1-1: 41328695G/A
1-1: 41375792C/T
1-1: 41384256C/A
1-1: 41384266T/C
1-1: 41384279T/C
1-1: 41384623C/G
1-1: 41402391A/C
1-1: 41412452T/A
1-1: 41503655G/A
1-1: 41505872T/A
1-1: 41537838A/C
1-1: 41551163G/A
1-1: 41566178T/C
1-1: 41586705C/G
1-1: 41648597C/T
1-1: 41700155C/T
1-1: 41742113T/A
1-1: 41752439C/A
1-1: 41764276A/T
1-1: 41810113A/G
1-1: 41836995A/T
1-1: 41872583C/T
1-1: 41906555T/C
1-1: 41932567G/A
1-1: 41966900G/T
1-1: 41982675G/T
1-1: 42135824T/C
1-1: 42241983T/C
1-1: 42339881T/A
1-1: 42348052C/A
1-1: 42409246G/T
1-1: 42442887G/T
1-1: 42510242T/C
1-1: 42526345T/C
1-1: 42559422T/A
1-1: 42632502G/A
1-1: 42701329C/G
1-1: 42715306G/A
1-1: 42748219T/C
1-1: 42793183T/C
1-1: 42819322T/C
1-1: 42820495T/C
1-1: 42821104G/A
1-1: 42875082A/G
1-1: 42885741T/A
1-1: 42907593G/C
1-1: 42925290T/C
1-1: 42971176C/A
1-1: 43229165T/C
1-1: 43343364C/T
1-1: 43400510C/T
1-1: 43444374C/T
1-1: 43447915C/A
1-1: 43448019C/T
1-1: 43455109C/T
1-1: 43471686G/T
1-1: 43472463A/G
1-1: 43485574A/G
1-1: 43583331C/A
1-1: 43641214A/T
1-1: 43657233C/T
1-1: 43681293T/C
1-1: 43746774A/C
1-1: 43757286A/C
1-1: 43828422A/T
1-1: 43856896A/C
1-1: 44060474C/G
1-1: 44068060G/A
1-1: 44096857C/T
1-1: 44151132A/T
1-1: 44161533C/G
1-1: 44244668C/A
1-1: 44310901T/C
1-1: 44311433T/G
1-1: 44325034T/C
1-1: 44325368T/A
1-1: 44341856C/T
1-1: 44360471G/A
1-1: 44436754T/C
1-1: 44456016A/T
1-1: 44532644C/A
1-1: 44579088A/G
1-1: 44678079C/T
1-1: 44698452C/T
1-1: 44711129G/C
1-1: 44759125A/T
1-1: 44924443T/C
1-1: 45034924A/T
1-1: 45059937A/T
1-1: 45096050A/G
1-1: 45140161T/C
1-1: 45223659A/G
1-1: 45245620C/T
1-1: 45298352A/G
1-1: 45332085C/T
1-1: 45356052T/C
1-1: 45372769A/T
1-1: 45414647G/A
1-1: 45450406G/A
1-1: 45530637T/G
1-1: 45545553A/T
1-1: 45566603C/T
1-1: 45579508T/C
1-1: 45665942C/G
1-1: 45745253G/A
1-1: 45781241T/A
1-1: 45807510A/G
1-1: 45851912A/G
1-1: 45907681T/A
1-1: 45919377T/G
1-1: 45930622C/A
1-1: 45941838T/C
1-1: 45952101C/A
1-1: 45952537A/C
1-1: 45963828T/C
1-1: 45992175C/A
1-1: 45995496G/A
1-1: 46003913A/T
1-1: 46015628A/T
1-1: 46032329T/G
1-1: 46053500C/G
1-1: 46069945C/A
1-1: 46123286C/G
1-1: 46150007T/G
1-1: 46168997C/T
1-1: 46211050C/T
1-1: 46243408G/A
1-1: 46274653T/G
1-1: 46274812A/G
1-1: 46278994T/A
1-1: 46285085G/A
1-1: 46288595G/T
1-1: 46288756A/T
1-1: 46289547A/T
1-1: 46311595T/C
1-1: 46313188C/T
1-1: 46327050A/G
1-1: 46416933A/C
1-1: 46475118G/A
1-1: 46492005T/A
1-1: 46511542T/A
1-1: 46521703G/A
1-1: 46590555A/T
1-1: 46603064C/T
1-1: 46637296G/T
1-1: 46657609G/A
1-1: 46668331A/C
1-1: 46722032G/A
1-1: 46785988C/T
1-1: 46798286C/T
1-1: 46838117C/T
1-1: 46874170A/G
1-1: 46874520A/G
1-1: 46901477G/C
1-1: 46901631T/G
1-1: 46909883G/A
1-1: 46909885A/G
1-1: 46963503G/C
1-1: 46973987T/G
1-1: 47119965G/A
1-1: 47169668G/A
1-1: 47187738A/T
1-1: 47199896C/T
1-1: 47314941T/C
1-1: 47340217G/A
1-1: 47411435C/T
1-1: 47452522G/A
1-1: 47499324G/T
1-1: 47567450G/A
1-1: 47639431A/G
1-1: 47676244T/A
1-1: 47731171C/A
1-1: 47850476C/T
1-1: 47851708T/A
1-1: 47902931G/A
1-1: 47952631T/C
1-1: 47990340G/A
1-1: 48070823C/T
1-1: 48071417T/A
1-1: 48111777G/T
1-1: 48140513T/A
1-1: 48151872A/G
1-1: 48153941G/C
1-1: 48154217C/T
1-1: 48204107G/A
1-1: 48205702A/T
1-1: 48205880C/A
1-1: 48230945A/T
1-1: 48272037C/T
1-1: 48371539G/A
1-1: 48427176A/C
1-1: 48532566C/A
1-1: 48606444G/A
1-1: 48644339C/G
1-1: 48670235C/A
1-1: 48862369T/C
1-1: 48864032G/A
1-1: 48938668A/G
1-1: 48969540A/G
1-1: 48997316C/T
1-1: 49064334T/C
1-1: 49083508G/C
1-1: 49083661T/A
1-1: 49083826T/A
1-1: 49084237A/G
1-1: 49084705A/G
1-1: 49099245G/T
1-1: 49099658A/G
1-1: 49102004T/C
1-1: 49116314T/C
1-1: 49157783G/A
1-1: 49190532A/G
1-1: 49226547C/G
1-1: 49263112C/A
1-1: 49292649C/T
1-1: 49385983A/C
1-1: 49461443C/T
1-1: 49491480A/G
1-1: 49575513T/G
1-1: 49585784A/G
1-1: 49604686G/A
1-1: 49633616C/T
1-1: 49656792C/T
1-1: 49689896G/A
1-1: 49744325G/C
1-1: 49839405G/C
1-1: 49870657T/C
1-1: 49909698C/T
1-1: 49954801G/C
1-1: 50045429A/T
1-1: 50092046G/T
1-1: 50141668C/T
1-1: 50146455A/T
1-1: 50153076G/A
1-1: 50153762C/G
1-1: 50153933T/C
1-1: 50154798C/T
1-1: 50157613C/T
1-1: 50159798A/C
1-1: 50159909A/C
1-1: 50174164G/A
1-1: 50184725A/C
1-1: 50227752C/T
1-1: 50363504A/T
1-1: 50468400C/T
1-1: 50554460G/T
1-1: 50612922T/C
1-1: 50779891G/T
1-1: 50783799T/C
1-1: 50796027G/T
1-1: 50864524T/C
1-1: 50896519C/T
1-1: 51065982G/A
1-1: 51075361A/T
1-1: 51088944G/A
1-1: 51130891A/T
1-1: 51202970T/A
1-1: 51257501C/T
1-1: 51315654G/A
1-1: 51352565A/G
1-1: 51397013T/G
1-1: 51478883C/A
1-1: 51518193C/A
1-1: 51522872A/G
1-1: 51592400G/A
1-1: 51646927A/C
1-1: 51661990C/A
1-1: 51685922C/T
1-1: 51855000G/A
1-1: 51869462C/T
1-1: 51924616C/T
1-1: 51967854C/T
1-1: 52099589T/C
1-1: 52185982G/A
1-1: 52213708T/C
1-1: 52233045G/A
1-1: 52330323A/G
1-1: 52331326G/A
1-1: 52384978C/T
1-1: 52385110T/A
1-1: 52401289A/G
1-1: 52401398A/G
1-1: 52401712T/C
1-1: 52465111T/G
1-1: 52502041T/A
1-1: 52531580A/C
1-1: 52565831T/C
1-1: 52602574T/G
1-1: 52702685C/T
1-1: 52764859G/C
1-1: 52808585G/A
1-1: 52843474C/T
1-1: 52885332A/C
1-1: 52904794G/A
1-1: 53074630T/A
1-1: 53086015A/T
1-1: 53395522T/A
1-1: 53506296G/.
1-1: 53516378C/T
1-1: 53538978T/G
1-1: 53562556G/A
1-1: 53589746A/G
1-1: 53646738C/G
1-1: 53651117G/A
1-1: 53651625C/T
1-1: 53653032A/T
1-1: 53653833A/G
1-1: 53653934G/T
1-1: 53653986A/T
1-1: 53674018T/C
1-1: 53723363A/C
1-1: 53723940C/T
1-1: 53758963A/C
1-1: 53810061C/A
1-1: 53843814T/A
1-1: 53862485G/A
1-1: 53993756A/G
1-1: 54017633G/A
1-1: 54044293G/A
1-1: 54114750T/A
1-1: 54142832C/T
1-1: 54238971T/C
1-1: 54308957G/T
1-1: 54363470C/A
1-1: 54415769T/A
1-1: 54461009C/T
1-1: 54538713A/G
1-1: 54587762C/T
1-1: 54700139T/C
1-1: 54702422G/A
1-1: 54733717C/T
1-1: 54746898G/A
1-1: 54825745T/G
1-1: 54951619T/C
1-1: 54952850G/A
1-1: 55084738C/T
1-1: 55124406A/T
1-1: 55135955A/G
1-1: 55142206G/A
1-1: 55180918G/T
1-1: 55196478A/G
1-1: 55198879C/T
1-1: 55214638C/A
1-1: 55276054G/A
1-1: 55277212C/A
1-1: 55278880C/T
1-1: 55279067A/G
1-1: 55279196G/C
1-1: 55279570C/T
1-1: 55285419A/G
1-1: 55285440T/C
1-1: 55285649A/T
1-1: 55285848C/T
1-1: 55286028C/T
1-1: 55286223T/C
1-1: 55286282A/G
1-1: 55286381C/G
1-1: 55286468A/T
1-1: 55286681G/A
1-1: 55287028A/T
1-1: 55287181C/T
1-1: 55287304G/T
1-1: 55287343C/T
1-1: 55287735G/A
1-1: 55295744G/A
1-1: 55298760C/T
1-1: 55315480A/G
1-1: 55424770G/A
1-1: 55426877A/G
1-1: 55443272A/G
1-1: 55571498G/C
1-1: 55783612C/T
1-1: 55798182G/A
1-1: 55801007C/G
1-1: 55818608C/T
1-1: 55845670T/G
1-1: 55937657C/T
1-1: 55954420G/A
1-1: 55980611C/A
1-1: 56045682T/C
1-1: 56056692G/A
1-1: 56096418C/G
1-1: 56160364C/T
1-1: 56268549A/G
1-1: 56299721T/A
1-1: 56316720C/T
1-1: 56333981T/C
1-1: 56423748T/G
1-1: 56497799T/G
1-1: 56538241T/A
1-1: 56581965A/C
1-1: 56642311G/A
1-1: 56686871T/A
1-1: 56731262A/G
1-1: 56806183G/T
1-1: 56811303A/G
1-1: 56811480T/C
1-1: 56935659A/G
1-1: 56937757G/A
1-1: 56956431G/A
1-1: 57030413T/A
1-1: 57097601T/G
1-1: 57144598G/T
1-1: 57162685T/A
1-1: 57164441A/T
1-1: 57165720T/G
1-1: 57198234G/T
1-1: 57351154G/A
1-1: 57363883A/T
1-1: 57372398G/T
1-1: 57372598C/T
1-1: 57374644C/T
1-1: 57472043A/G
1-1: 57506417G/T
1-1: 57514356C/T
1-1: 57573614C/G
1-1: 57593221G/A
1-1: 57639399G/A
1-1: 57758743T/C
1-1: 57795349G/A
1-1: 57816764C/T
1-1: 57945440C/A
1-1: 57998322G/C
1-1: 58051864T/C
1-1: 58110431G/A
1-1: 58155084C/A
1-1: 58224201G/C
1-1: 58227161G/A
1-1: 58227444T/C
1-1: 58296624T/A
1-1: 58317984A/C
1-1: 58485770A/G
1-1: 58511267C/T
1-1: 58587371G/T
1-1: 58687740A/C
1-1: 58746697G/A
1-1: 58781806G/T
1-1: 58902473G/C
1-1: 58922359A/G
1-1: 58941035G/A
1-1: 58978894T/A
1-1: 59015851A/G
1-1: 59146456C/G
1-1: 59216496T/C
1-1: 59251930C/G
1-1: 59285521G/A
1-1: 59364948T/C
1-1: 59450959C/T
1-1: 59574464C/A
1-1: 59575938G/A
1-1: 59586376C/G
1-1: 59588046A/G
1-1: 59590962T/C
1-1: 59598117G/A
1-1: 59628143C/T
1-1: 59682696G/C
1-1: 59731497C/G
1-1: 59759392C/A
1-1: 59793161G/A
1-1: 59808278T/A
1-1: 59823296G/A
1-1: 59890136T/C
1-1: 59911391G/A
1-1: 59942388T/C
1-1: 60013603T/A
1-1: 60131390A/G
1-1: 60212993C/T
1-1: 60213227T/C
1-1: 60252132G/A
1-1: 60285614A/G
1-1: 60324444C/T
1-1: 60341456C/T
1-1: 60460050T/A
1-1: 60556024G/A
1-1: 60599558T/G
1-1: 60639156A/G
1-1: 60757833G/C
1-1: 60761124T/C
1-1: 60781136T/A
1-1: 60792971G/A
1-1: 60917685G/T
1-1: 60973116T/C
1-1: 60996944C/A
1-1: 61127453A/T
1-1: 61313362G/A
1-1: 61340954A/G
1-1: 61367013C/T
1-1: 61574599G/T
1-1: 61621361C/G
1-1: 61668411G/A
1-1: 61713565A/G
1-1: 61720721A/G
1-1: 61742924A/G
1-1: 61759987T/A
1-1: 61869938A/T
1-1: 61899434C/G
1-1: 62022855T/A
1-1: 62126431T/C
1-1: 62327043C/A
1-1: 62440446C/A
1-1: 62502780A/G
1-1: 62518175T/C
1-1: 62558679G/A
1-1: 62611300G/A
1-1: 62626451G/A
1-1: 62709547G/T
1-1: 62709930A/G
1-1: 62798501C/T
1-1: 62825386C/T
1-1: 62853531G/A
1-1: 62861111A/T
1-1: 62885544A/T
1-1: 62896337T/C
1-1: 62897797T/A
1-1: 62897899G/C
1-1: 62897906A/T
1-1: 62899152T/G
1-1: 62899659G/T
1-1: 62899690T/C
1-1: 62906756T/G
1-1: 62945856C/T
1-1: 63100509A/G
1-1: 63145338T/C
1-1: 63217735T/C
1-1: 63274521T/C
1-1: 63371190C/G
1-1: 63375763C/T
1-1: 63479617A/G
1-1: 63543179G/A
1-1: 63576398T/C
1-1: 63578751C/T
1-1: 63584611T/C
1-1: 63586536C/T
1-1: 63591122T/C
1-1: 63613016A/G
1-1: 63653567C/T
1-1: 63691326C/T
1-1: 63706430A/G
1-1: 63720173T/A
1-1: 63845069A/C
1-1: 64028092G/T
1-1: 64044753A/G
1-1: 64055852G/C
1-1: 64250273G/A
1-1: 64303590A/G
1-1: 64323727C/A
1-1: 64417874A/G
1-1: 64418708A/G
1-1: 64428477T/G
1-1: 64440588G/A
1-1: 64456724A/T
1-1: 64462011C/T
1-1: 64475686C/A
1-1: 64487990T/G
1-1: 64503000G/A
1-1: 64504722G/A
1-1: 64507436T/A
1-1: 64507545C/T
1-1: 64513314G/T
1-1: 64533411T/C
1-1: 64535787T/C
1-1: 64560698G/C
1-1: 64571413C/G
1-1: 64584730A/T
1-1: 64596907T/A
1-1: 64626145C/T
1-1: 64661545T/G
1-1: 64672666G/A
1-1: 64688765T/C
1-1: 64746904C/A
1-1: 64790380A/C
1-1: 64849288C/G
1-1: 64883217G/A
1-1: 64960387A/C
1-1: 65009550T/G
1-1: 65044623T/G
1-1: 65095842T/C
1-1: 65195142C/T
1-1: 65221544A/T
1-1: 65257832A/G
1-1: 65520354T/C
1-1: 65573196G/T
1-1: 65642214C/T
1-1: 65730239A/C
1-1: 65779126G/A
1-1: 65781861C/A
1-1: 65842341A/G
1-1: 65881290C/T
1-1: 65928570T/C
1-1: 65986546C/G
1-1: 66028585C/T
1-1: 66111082A/T
1-1: 66118453G/A
1-1: 66131721T/A
1-1: 66147013G/C
1-1: 66281592A/T
1-1: 66432350A/G
1-1: 66506515T/A
1-1: 66778428A/G
1-1: 66799102C/T
1-1: 66843479G/A
1-1: 66857130G/A
1-1: 67004466T/C
1-1: 67070000T/A
1-1: 67257203T/A
1-1: 67301432T/G
1-1: 67339399G/T
1-1: 67373881C/T
1-1: 67439714C/A
1-1: 67475629C/T
1-1: 67662367C/T
1-1: 67713569C/G
1-1: 67723034C/T
1-1: 67792010T/C
1-1: 67849084C/T
1-1: 67899384G/C
1-1: 67976326C/T
1-1: 68001439T/G
1-1: 68036924A/T
1-1: 68077402G/T
1-1: 68126977T/A
1-1: 68174191C/A
1-1: 68217439G/A
1-1: 68267711T/C
1-1: 68370197G/A
1-1: 68424445A/T
1-1: 68505646T/G
1-1: 68539408T/C
1-1: 68552140T/A
1-1: 68573518T/A
1-1: 68590181C/G
1-1: 68590375G/A
1-1: 68600702C/T
1-1: 68657428T/C
1-1: 68736125T/A
1-1: 68823872G/A
1-1: 68895979G/A
1-1: 69070838C/T
1-1: 69071101C/T
1-1: 69071108G/A
1-1: 69214064G/A
1-1: 69240730A/G
1-1: 69276197A/G
1-1: 69276839C/T
1-1: 69331097G/T
1-1: 69427108C/G
1-1: 69485969A/G
1-1: 69566219G/C
1-1: 69646557G/T
1-1: 69833327C/G
1-1: 69932501A/G
1-1: 70030935A/G
1-1: 70071133C/T
1-1: 70101275A/G
1-1: 70156214T/C
1-1: 70218979T/C
1-1: 70318679G/A
1-1: 70374118T/C
1-1: 70524040A/T
1-1: 70524223G/C
1-1: 70551524A/C
1-1: 70579221A/G
1-1: 70609679C/A
1-1: 70660760A/G
1-1: 70774522T/A
1-1: 70801183A/T
1-1: 70822883C/A
1-1: 70846034C/A
1-1: 70909246A/G
1-1: 70987705A/T
1-1: 71026734A/T
1-1: 71029471C/T
1-1: 71064958G/A
1-1: 71070434T/G
1-1: 71070455G/T
1-1: 71073723C/G
1-1: 71073803C/A
1-1: 71106758G/A
1-1: 71152467A/T
1-1: 71152500C/T
1-1: 71152519C/T
1-1: 71152632G/A
1-1: 71195944C/T
1-1: 71269528T/C
1-1: 71297725C/T
1-1: 71428210T/A
1-1: 71428987G/A
1-1: 71656004C/T
1-1: 71824559A/G
1-1: 71926990A/G
1-1: 71944056C/T
1-1: 72125105A/T
1-1: 72130439G/C
1-1: 72141809T/C
1-1: 72256542C/A
1-1: 72257884T/G
1-1: 72288461G/A
1-1: 72340872A/G
1-1: 72426299C/T
1-1: 72452238G/A
1-1: 72481062C/T
1-1: 72533286C/T
1-1: 72583466T/C
1-1: 72656936C/T
1-1: 72777888T/C
1-1: 72790666C/T
1-1: 72852810C/T
1-1: 72970881T/G
1-1: 73005652G/A
1-1: 73011576A/G
1-1: 73065686G/C
1-1: 73095030T/G
1-1: 73191913A/T
1-1: 73317049C/T
1-1: 73399442G/A
1-1: 73496899T/G
1-1: 73515929C/T
1-1: 73535247G/A
1-1: 73550660T/C
1-1: 73610129G/A
1-1: 73756500C/T
1-1: 73774217G/A
1-1: 73821599C/A
1-1: 73879799G/C
1-1: 73889085T/C
1-1: 73906751G/T
1-1: 74067520T/C
1-1: 74182916G/T
1-1: 74271480T/A
1-1: 74287376G/A
1-1: 74307513C/A
1-1: 74381766C/T
1-1: 74399269T/A
1-1: 74426635T/G
1-1: 74449307C/T
1-1: 74453629C/A
1-1: 74461779G/C
1-1: 74467398G/A
1-1: 74504761T/G
1-1: 74509010T/C
1-1: 74509246T/C
1-1: 74509370A/G
1-1: 74509403A/G
1-1: 74509476C/G
1-1: 74511586A/G
1-1: 74513372A/T
1-1: 74513478C/T
1-1: 74513724C/A
1-1: 74514085T/C
1-1: 74514154T/C
1-1: 74515099T/C
1-1: 74515138T/C
1-1: 74515382C/A
1-1: 74517540T/C
1-1: 74517650A/G
1-1: 74517970C/G
1-1: 74518399T/A
1-1: 74537663A/G
1-1: 74538003A/G
1-1: 74538211T/C
1-1: 74538620A/C
1-1: 74539381C/T
1-1: 74545540A/G
1-1: 74545550C/T
1-1: 74545568G/A
1-1: 74545761C/T
1-1: 74547952G/A
1-1: 74561455T/C
1-1: 74573385C/T
1-1: 74573784C/T
1-1: 74573954A/G
1-1: 74588345A/G
1-1: 74591146T/A
1-1: 74591311C/T
1-1: 74599277C/A
1-1: 74604272T/C
1-1: 74604300G/C
1-1: 74604858A/C
1-1: 74667241C/T
1-1: 74722397T/C
1-1: 74821056T/A
1-1: 74961227G/A
1-1: 74962096A/G
1-1: 75059940T/A
1-1: 75118392T/C
1-1: 75147888C/T
1-1: 75191475A/G
1-1: 75210445A/G
1-1: 75382844C/T
1-1: 75442592A/G
1-1: 75490328C/T
1-1: 75540826A/G
1-1: 75627227C/A
1-1: 75627860C/A
1-1: 75641628A/G
1-1: 75659085A/G
1-1: 75735508A/T
1-1: 75785389G/C
1-1: 75798622T/C
1-1: 76025791G/A
1-1: 76256411G/A
1-1: 76308642A/G
1-1: 76347134G/A
1-1: 76441558G/A
1-1: 76705785T/A
1-1: 76726301G/A
1-1: 76937839T/G
1-1: 77152739C/T
1-1: 77175543C/T
1-1: 77689597C/T
1-1: 77908664T/A
1-1: 78051760A/T
1-1: 78113396A/T
1-1: 78430771G/A
1-1: 78438595G/A
1-1: 78471380G/A
1-1: 78471592G/A
1-1: 78566544T/A
1-1: 78580973A/G
1-1: 78629830C/A
1-1: 78797729T/C
1-1: 78876427C/T
1-1: 78939446C/T
1-1: 79060880T/C
1-1: 79069021T/A
1-1: 79079081G/T
1-1: 79146581A/T
1-1: 79212775C/T
1-1: 79285631C/A
1-1: 79313596A/G
1-1: 79347255C/T
1-1: 79440735G/A
1-1: 79584307A/T
1-1: 79607484T/C
1-1: 79872353T/C
1-1: 79927165A/T
1-1: 79983783C/T
1-1: 80214472C/T
1-1: 81075465T/G
1-1: 81307428T/C
1-2: 554864T/A
1-2: 738722A/T
1-2: 892248G/T
1-2: 1026668G/A
1-2: 1102315C/T
1-2: 1122030T/A
1-2: 1176398A/G
1-2: 1656739G/C
1-2: 1674492A/G
1-2: 1702078A/C
1-2: 1819067A/C
1-2: 2013311G/A
1-2: 2014338A/G
1-2: 2066615T/A
1-2: 2135816G/A
1-2: 2217734G/A
1-2: 2295706A/C
1-2: 2329557T/C
1-2: 2363456C/T
1-2: 2372133G/T
1-2: 2382518C/T
1-2: 2397435C/T
1-2: 2398088T/G
1-2: 2430588T/C
1-2: 2431154T/C
1-2: 2431351T/A
1-2: 2431368C/T
1-2: 2432495C/T
1-2: 2432558T/G
1-2: 2433120A/G
1-2: 2433336G/A
1-2: 2433632G/A
1-2: 2433648C/A
1-2: 2433892C/A
1-2: 2434059T/C
1-2: 2434303G/A
1-2: 2434725T/G
1-2: 2452210A/T
1-2: 2452228A/T
1-2: 2486633A/C
1-2: 2564137A/C
1-2: 2782222T/G
1-2: 2809423A/T
1-2: 2843873G/A
1-2: 2908688C/T
1-2: 2938785G/A
1-2: 3011588G/T
1-2: 3277873T/C
1-2: 3633442A/G
1-2: 3719857T/C
1-2: 3747300G/C
1-2: 3800639T/G
1-2: 3966409A/T
1-2: 3966923T/A
1-2: 3970025A/G
1-2: 4012481G/A
1-2: 4034832G/A
1-2: 4178072G/T
1-2: 4191030C/T
1-2: 4209832T/A
1-2: 4297426T/A
1-2: 4416147A/C
1-2: 4629656T/C
1-2: 4917703C/G
1-2: 4935704T/A
1-2: 4950039C/T
1-2: 5097297A/G
1-2: 5118969T/C
1-2: 5194099A/G
1-2: 5412711C/T
1-2: 5421497C/T
1-2: 5433916C/T
1-2: 5455651G/C
1-2: 5471320T/C
1-2: 5481690C/T
1-2: 5482127G/T
1-2: 5489256C/G
1-2: 5489541G/T
1-2: 5490891T/G
1-2: 5490893T/G
1-2: 5492013C/T
1-2: 5492240G/T
1-2: 5501821A/G
1-2: 5501822T/C
1-2: 5502494A/G
1-2: 5513161T/G
1-2: 5733394A/G
1-2: 5809280G/A
1-2: 5862511T/C
1-2: 5952556T/C
1-2: 5983985A/G
1-2: 6121341T/C
1-2: 6278351T/A
1-2: 6320274C/A
1-2: 6384479A/G
1-2: 6429428C/A
1-2: 6429901T/C
1-2: 6433095G/A
1-2: 6484265A/G
1-2: 6668078C/T
1-2: 6727042G/A
1-2: 6813048G/A
1-2: 6828224G/A
1-2: 6971684A/G
1-2: 7279317C/T
1-2: 7338593A/G
1-2: 7369381C/T
1-2: 7395484A/G
1-2: 7406730A/G
1-2: 7580688G/C
1-2: 7640836A/G
1-2: 7697705T/C
1-2: 7703122G/A
1-2: 7703226A/G
1-2: 7704377G/C
1-2: 7704535T/A
1-2: 7704708G/A
1-2: 7704920A/G
1-2: 7704929C/A
1-2: 7705094T/C
1-2: 7771885G/T
1-2: 7777094G/A
1-2: 7777235C/T
1-2: 7777865C/T
1-2: 7786641A/G
1-2: 7868316T/C
1-2: 7886956G/A
1-2: 7906748A/T
1-2: 7963997G/T
1-2: 7995010G/A
1-2: 7998087G/C
1-2: 7998308C/T
1-2: 8017517T/A
1-2: 8065420C/T
1-2: 8080369G/C
1-2: 8095762C/G
1-2: 8097463G/A
1-2: 8100165T/C
1-2: 8102097T/G
1-2: 8104955T/C
1-2: 8106854T/A
1-2: 8145409C/T
1-2: 8160782G/T
1-2: 8173453A/G
1-2: 8186988T/A
1-2: 8194580T/G
1-2: 8274989G/C
1-2: 8279216G/A
1-2: 8352864T/G
1-2: 8354532A/G
1-2: 8403754A/C
1-2: 8447701C/T
1-2: 8464623T/C
1-2: 8507384G/T
1-2: 8582764A/T
1-2: 8671035T/C
1-2: 8686291G/A
1-2: 8788946A/T
1-2: 8789028A/G
1-2: 8947304T/G
1-2: 8988887C/T
1-2: 9006803T/G
1-2: 9081965C/T
1-2: 9114114C/T
1-2: 9160107C/T
1-2: 9199252T/C
1-2: 9251044G/C
1-2: 9261317G/C
1-2: 9357042G/T
1-2: 9392680A/G
1-2: 9475645T/C
1-2: 9519786T/A
1-2: 9537527T/G
1-2: 9579525A/G
1-2: 9640868A/G
1-2: 9641026C/T
1-2: 9641686T/A
1-2: 9732232T/C
1-2: 9774059T/A
1-2: 9826540G/A
1-2: 9827166C/T
1-2: 9830796G/A
1-2: 9832409G/C
1-2: 9832674A/G
1-2: 9832794C/T
1-2: 9834828C/T
1-2: 9834928C/A
1-2: 9835034T/C
1-2: 9836617A/C
1-2: 9836696A/G
1-2: 9836748C/T
1-2: 9837668C/T
1-2: 9838835G/T
1-2: 9838884G/C
1-2: 9839071C/A
1-2: 9839077T/A
1-2: 9839654C/T
1-2: 9839765A/G
1-2: 9839910C/T
1-2: 9839975A/C
1-2: 9840065T/C
1-2: 9840802G/A
1-2: 9841567C/T
1-2: 9841746C/T
1-2: 9842452T/C
1-2: 9875221C/T
1-2: 9885076G/A
1-2: 9918447A/C
1-2: 9952370G/A
1-2: 9998188C/A
1-2: 10008190A/T
1-2: 10135162C/T
1-2: 10172393T/A
1-2: 10202512T/C
1-2: 10250795G/A
1-2: 10326364A/T
1-2: 10359761G/A
1-2: 10423241A/T
1-2: 10492707C/T
1-2: 10572037T/C
1-2: 10752219C/T
1-2: 10848005G/C
1-2: 10864429C/T
1-2: 10912917T/C
1-2: 10924636T/C
1-2: 10950985C/T
1-2: 11000743T/A
1-2: 11054986G/A
1-2: 11080258C/T
1-2: 11120461A/T
1-2: 11170855G/A
1-2: 11197373G/A
1-2: 11245134T/A
1-2: 11272953A/G
1-2: 11290045G/T
1-2: 11290287C/A
1-2: 11345276A/G
1-2: 11380081C/G
1-2: 11590358C/T
1-2: 11650499C/G
1-2: 11670184C/T
1-2: 11680079T/C
1-2: 11683820T/A
1-2: 11708520A/T
1-2: 11719369A/T
1-2: 11745943T/C
1-2: 11777875T/C
1-2: 11784680G/T
1-2: 11816773G/A
1-2: 11899361A/G
1-2: 11921556A/G
1-2: 12004385T/C
1-2: 12178868A/C
1-2: 12239571G/A
1-2: 12507904C/T
1-2: 12649327C/G
1-2: 12660636A/G
1-2: 12697688G/A
1-2: 12735366T/C
1-2: 12825154C/G
1-2: 12995643C/G
1-2: 13060761T/C
1-2: 13149673A/G
1-2: 13229660A/T
1-2: 13234450A/T
1-2: 13239731A/G
1-2: 13277637C/T
1-2: 13325969A/G
1-2: 13359804A/T
1-2: 13413280T/C
1-2: 13434218G/T
1-2: 13469446C/T
1-2: 13557386A/G
1-2: 13599106A/G
1-2: 13612033T/C
1-2: 13644503A/G
1-2: 13690004G/A
1-2: 13724677C/A
1-2: 13746395G/A
1-2: 13767002A/G
1-2: 13799269T/C
1-2: 13820049T/C
1-2: 13843228T/C
1-2: 13863584C/T
1-2: 13875315C/T
1-2: 13961152T/C
1-2: 14024774A/G
1-2: 14084075C/T
1-2: 14098221G/T
1-2: 14117619C/T
1-2: 14272273C/G
1-2: 14272401A/T
1-2: 14287547T/C
1-2: 14327500G/T
1-2: 14377501C/T
1-2: 14394982C/T
1-2: 14416858T/A
1-2: 14437617C/T
1-2: 14527462T/A
1-2: 14578165T/G
1-2: 14641238T/C
1-2: 14815752A/G
1-2: 14837592G/A
1-2: 14951763C/T
1-2: 14982175G/A
1-2: 15006911A/C
1-2: 15060511C/T
1-2: 15086419C/A
1-2: 15188026C/T
1-2: 15243555T/G
1-2: 15278719C/T
1-2: 15350555A/C
1-2: 15507003T/C
1-2: 15566330G/T
1-2: 15606012A/G
1-2: 15615869G/A
1-2: 15616215C/G
1-2: 15616380G/T
1-2: 15616517T/C
1-2: 15626800A/T
1-2: 15629789G/A
1-2: 15629859C/T
1-2: 15629914T/C
1-2: 15630040G/A
1-2: 15637345T/C
1-2: 15642346C/G
1-2: 15654692T/C
1-2: 15655334G/A
1-2: 15662778A/G
1-2: 15668786T/C
1-2: 15703732A/T
1-2: 15706139T/G
1-2: 15730913G/T
1-2: 15739442C/A
1-2: 15790424A/G
1-2: 15835724A/T
1-2: 15972624A/G
1-2: 15981858T/A
1-2: 16036417G/A
1-2: 16046307A/G
1-2: 16057856C/A
1-2: 16057958T/C
1-2: 16058069C/T
1-2: 16058189C/T
1-2: 16058238A/G
1-2: 16058312T/C
1-2: 16058459T/G
1-2: 16066066A/G
1-2: 16066193T/C
1-2: 16066913G/A
1-2: 16066967C/T
1-2: 16066996C/T
1-2: 16069440G/T
1-2: 16130744A/G
1-2: 16173083A/G
1-2: 16236265C/A
1-2: 16288300A/G
1-2: 16395732T/G
1-2: 16433238C/T
1-2: 16507347T/C
1-2: 16637508C/T
1-2: 16644980G/A
1-2: 16739829A/G
1-2: 16791497G/T
1-2: 16820319G/A
1-2: 16863072C/T
1-2: 16934311C/A
1-2: 16957052C/T
1-2: 16997893A/G
1-2: 17074331C/T
1-2: 17112464T/C
1-2: 17112628A/G
1-2: 17266604C/A
1-2: 17293573G/C
1-2: 17297297T/C
1-2: 17310724C/G
1-2: 17368315C/A
1-2: 17399668A/G
1-2: 17427940C/T
1-2: 17459133C/A
1-2: 17474913A/C
1-2: 17503820G/A
1-2: 17532803T/C
1-2: 17548298G/T
1-2: 17588058G/A
1-2: 17669285T/C
1-2: 17715603T/A
1-2: 17755673C/T
1-2: 17756673C/G
1-2: 17792341G/T
1-2: 17819935T/C
1-2: 17920020A/T
1-2: 18177822G/A
1-2: 18225151G/A
1-2: 18225383A/G
1-2: 18253308T/A
1-2: 18280874C/A
1-2: 18327714G/A
1-2: 18355140C/A
1-2: 18381816A/T
1-2: 18439653C/T
1-2: 18457089A/G
1-2: 18482826A/G
1-2: 18492866C/A
1-2: 18504396C/T
1-2: 18526868A/G
1-2: 18576885C/T
1-2: 18631494T/C
1-2: 18632536T/C
1-2: 18632563G/T
1-2: 18632569T/C
1-2: 18633767T/C
1-2: 18633812T/C
1-2: 18676207G/A
1-2: 18701422T/A
1-2: 18739579A/G
1-2: 18829936G/T
1-2: 18893514C/A
1-2: 18999169G/A
1-2: 19003767A/G
1-2: 19025786C/G
1-2: 19035651T/C
1-2: 19036994A/G
1-2: 19156513T/A
1-2: 19234466T/C
1-2: 19381010C/G
1-2: 19440119A/T
1-2: 19486945C/G
1-2: 19488315T/C
1-2: 19582150T/C
1-2: 19609241T/C
1-2: 19654725C/A
1-2: 19689833C/A
1-2: 19709659G/A
1-2: 19814905C/T
1-2: 19826816A/C
1-2: 19911603A/T
1-2: 19978078A/G
1-2: 19988125C/T
1-2: 19989200A/G
1-2: 20021748C/T
1-2: 20052268T/G
1-2: 20063022T/C
1-2: 20074643C/A
1-2: 20124973T/C
1-2: 20194792T/C
1-2: 20337948T/C
1-2: 20379941T/C
1-2: 20382268C/T
1-2: 20417382T/C
1-2: 20508189G/A
1-2: 20545022C/T
1-2: 20561124T/C
1-2: 20645315C/T
1-2: 20646682T/G
1-2: 20677456C/A
1-2: 20858689A/G
1-2: 20952066T/C
1-2: 20956859G/C
1-2: 20957168C/T
1-2: 20991800C/T
1-2: 21002195T/A
1-2: 21073707C/T
1-2: 21111342A/T
1-2: 21171737C/T
1-2: 21199708C/A
1-2: 21210254G/C
1-2: 21227994C/T
1-2: 21249871T/G
1-2: 21250062T/A
1-2: 21340872G/C
1-2: 21389543G/C
1-2: 21420438A/T
1-2: 21459555T/C
1-2: 21471617G/A
1-2: 21475163A/T
1-2: 21475179G/A
1-2: 21494014C/A
1-2: 21494022C/T
1-2: 21513139A/C
1-2: 21523252G/A
1-2: 21541645A/G
1-2: 21584111C/G
1-2: 21646764G/C
1-2: 21722706G/A
1-2: 21738108G/A
1-2: 21832911A/G
1-2: 21900499G/T
1-2: 21977968A/T
1-2: 22071176T/C
1-2: 22089039G/A
1-2: 22140931G/T
1-2: 22177632G/A
1-2: 22220645T/C
1-2: 22245774G/A
1-2: 22275721G/A
1-2: 22285575G/T
1-2: 22310943G/C
1-2: 22380328T/A
1-2: 22382227G/T
1-2: 22431413C/T
1-2: 22449368A/G
1-2: 22458964G/A
1-2: 22459054C/T
1-2: 22463718C/T
1-2: 22463737C/T
1-2: 22463972G/A
1-2: 22464240T/G
1-2: 22464355C/T
1-2: 22464580G/T
1-2: 22464645A/T
1-2: 22488357C/T
1-2: 22529146C/T
1-2: 22591903C/A
1-2: 22597948C/A
1-2: 22614737A/.
1-2: 22640906A/C
1-2: 22640924C/A
1-2: 22641077T/G
1-2: 22641184C/T
1-2: 22643968C/G
1-2: 22655850C/T
1-2: 22663561A/G
1-2: 22663694A/G
1-2: 22663723C/G
1-2: 22663818C/T
1-2: 22663827T/C
1-2: 22664053A/G
1-2: 22664077T/C
1-2: 22664291A/G
1-2: 22664905T/C
1-2: 22668471C/T
1-2: 22681378G/A
1-2: 22682053G/.
1-2: 22705895T/G
1-2: 22725005A/G
1-2: 22780031C/G
1-2: 22841880T/C
1-2: 22860812C/A
1-2: 22889868G/C
1-2: 22957906A/C
1-2: 23041390G/A
1-2: 23081099T/C
1-2: 23202601T/G
1-2: 23213408G/T
1-2: 23232900G/A
1-2: 23268165A/T
1-2: 23344606T/G
1-2: 23366564T/A
1-2: 23391695C/T
1-2: 23430622C/T
1-2: 23530896C/A
1-2: 23573969G/A
1-2: 23643233C/G
1-2: 23658111C/A
1-2: 23684763T/A
1-2: 23720402T/C
1-2: 23783095G/T
1-2: 23806940A/T
1-2: 23817490C/T
1-2: 23823705T/C
1-2: 23825026G/A
1-2: 23825298A/G
1-2: 23825307C/A
1-2: 23825705A/G
1-2: 23826029A/G
1-2: 23845440C/T
1-2: 23899627T/C
1-2: 23935009G/A
1-2: 24010770C/T
1-2: 24040606G/A
1-2: 24061186T/A
1-2: 24102635A/G
1-2: 24112513A/G
1-2: 24113731A/T
1-2: 24132943A/G
1-2: 24181064G/A
1-2: 24184501G/A
1-2: 24198527G/T
1-2: 24227047A/G
1-2: 24270938A/T
1-2: 24300349C/T
1-2: 24445601T/C
1-2: 24452937T/G
1-2: 24554282T/C
1-2: 24581162T/C
1-2: 24611960G/A
1-2: 24651466T/G
1-2: 24705478T/C
1-2: 24757923G/A
1-2: 24820726A/T
1-2: 24849521A/G
1-2: 24993234A/G
1-2: 25019482C/A
1-2: 25069575A/C
1-2: 25118586A/T
1-2: 25118868A/G
1-2: 25118886C/T
1-2: 25180773C/T
1-2: 25332087C/A
1-2: 25332167T/C
1-2: 25345927A/T
1-2: 25439141C/T
1-2: 25477397T/A
1-2: 25487446G/A
1-2: 25546263A/G
1-2: 25548281T/C
1-2: 25576566A/G
1-2: 25576892T/C
1-2: 25577364T/C
1-2: 25586652T/C
1-2: 25603915C/T
1-2: 25618475A/G
1-2: 25798416C/A
1-2: 25804187A/G
1-2: 25850494T/C
1-2: 25879376G/A
1-2: 25915936T/C
1-2: 25990289T/A
1-2: 26000304T/C
1-2: 26039818T/A
1-2: 26077906C/T
1-2: 26136828A/C
1-2: 26149833T/C
1-2: 26206948C/A
1-2: 26222949T/C
1-2: 26417395G/A
1-2: 26635650T/C
1-2: 26772792T/C
1-2: 26830444T/C
1-2: 26878995C/T
1-2: 26916114A/G
1-2: 26929139C/T
1-2: 26977935A/C
1-2: 27050645C/T
1-2: 27061838G/A
1-2: 27084685C/T
1-2: 27097971T/C
1-2: 27130785C/T
1-2: 27175633G/T
1-2: 27257383A/C
1-2: 27268275C/A
1-2: 27270756T/C
1-2: 27275519C/G
1-2: 27286620T/C
1-2: 27290975T/C
1-2: 27381950A/G
1-2: 27412668G/A
1-2: 27441651A/C
1-2: 27469227C/A
1-2: 27546466C/A
1-2: 27562014G/A
1-2: 27607525G/A
1-2: 27658874A/G
1-2: 27706093G/T
1-2: 27750581T/C
1-2: 27791745A/C
1-2: 27825912G/A
1-2: 27973018A/T
1-2: 28008687G/A
1-2: 28101530C/T
1-2: 28117655G/A
1-2: 28207559A/G
1-2: 28250643T/C
1-2: 28320083C/T
1-2: 28328869G/A
1-2: 28341450A/G
1-2: 28352802G/A
1-2: 28368350A/C
1-2: 28378451A/G
1-2: 28394578T/A
1-2: 28434449C/T
1-2: 28507853A/G
1-2: 28541824G/A
1-2: 28541928A/G
1-2: 28613902C/T
1-2: 28707302C/T
1-2: 28733883C/T
1-2: 28784893T/A
1-2: 28809856T/A
1-2: 28810222C/T
1-2: 28894678C/G
1-2: 29058260G/T
1-2: 29133882G/A
1-2: 29181407G/C
1-2: 29192273T/A
1-2: 29210398C/G
1-2: 29291810C/T
1-2: 29317396C/T
1-2: 29330411G/A
1-2: 29361634G/A
1-2: 29518465A/T
1-2: 29562486G/A
1-2: 29648330C/G
1-2: 29786840T/A
1-2: 29816889C/T
1-2: 29835105T/C
1-2: 29846902A/C
1-2: 29850906C/T
1-2: 29850965A/G
1-2: 29850973G/A
1-2: 29850998T/C
1-2: 29851025C/T
1-2: 29851058T/C
1-2: 29851061A/G
1-2: 29851073A/G
1-2: 29851155G/A
1-2: 30016858G/A
1-2: 30066055C/T
1-2: 30090999C/A
1-2: 30112744A/G
1-2: 30202106G/A
1-2: 30212525A/G
1-2: 30345277A/G
1-2: 30353183T/C
1-2: 30378024A/G
1-2: 30420225G/A
1-2: 30445056T/A
1-2: 30448889T/C
1-2: 30542572T/A
1-2: 30542878G/A
1-2: 30542891T/C
1-2: 30543069T/G
1-2: 30543176C/A
1-2: 30543333C/A
1-2: 30543837G/C
1-2: 30544076G/A
1-2: 30544090A/G
1-2: 30544127T/A
1-2: 30555062G/C
1-2: 30583508T/A
1-2: 30616081C/T
1-2: 30649382G/A
1-2: 30735919A/T
1-2: 30762952A/G
1-2: 30799718T/C
1-2: 30936969A/C
1-2: 30953613T/C
1-2: 30968832T/C
1-2: 30968834C/T
1-2: 30984045C/T
1-2: 31028731T/G
1-2: 31094413A/G
1-2: 31203468A/T
1-2: 31283625A/G
1-2: 31295538C/T
1-2: 31295639A/G
1-2: 31295886C/G
1-2: 31306798C/T
1-2: 31372297C/T
1-2: 31388946C/T
1-2: 31427712C/T
1-2: 31484207T/C
1-2: 31503690C/G
1-2: 31637583A/G
1-2: 31707162A/G
1-2: 31728960C/T
1-2: 31782633G/A
1-2: 31845114G/C
1-2: 31867226A/G
1-2: 31867235T/C
1-2: 31867236C/A
1-2: 31867238C/T
1-2: 31867283T/C
1-2: 31867415T/G
1-2: 31875946C/A
1-2: 31894518C/T
1-2: 31910683C/T
1-2: 31942997T/C
1-2: 31954785A/T
1-2: 31983714C/T
1-2: 32059739C/T
1-2: 32127039T/A
1-2: 32203515A/G
1-2: 32231300G/T
1-2: 32266673A/.
1-2: 32266677T/.
1-2: 32267767T/C
1-2: 32267780A/G
1-2: 32267804G/T
1-2: 32267813G/A
1-2: 32271282T/G
1-2: 32287242C/T
1-2: 32325139C/T
1-2: 32335870A/G
1-2: 32417384A/T
1-2: 32447073T/A
1-2: 32482817G/C
1-2: 32570211A/T
1-2: 32585638G/A
1-2: 32596000G/C
1-2: 32612752C/T
1-2: 32627385G/C
1-2: 32642947A/G
1-2: 32643594G/T
1-2: 32677135G/A
1-2: 32699338G/C
1-2: 32746348G/T
1-2: 32774324C/T
1-2: 32903366A/G
1-2: 32910546A/G
1-2: 32924034A/G
1-2: 33008588C/G
1-2: 33067629G/T
1-2: 33141296C/G
1-2: 33309266G/A
1-2: 33316499C/T
1-2: 33337122A/G
1-2: 33371370G/C
1-2: 33456330A/G
1-2: 33516124A/G
1-2: 33529119C/A
1-2: 33546328G/A
1-2: 33614722T/C
1-2: 33620397C/T
1-2: 33710569T/C
1-2: 33736950G/A
1-2: 33765252T/C
1-2: 33822341G/T
1-2: 33919236G/A
1-2: 33986452T/C
1-2: 33987953C/A
1-2: 34042552G/A
1-2: 34043029G/T
1-2: 34132277T/C
1-2: 34161306A/C
1-2: 34295797A/G
1-2: 34296941C/T
1-2: 34367770C/T
1-2: 34406633T/G
1-2: 34479538C/A
1-2: 34884123G/T
1-2: 35061663C/T
1-2: 35261063C/T
1-2: 35265397C/T
1-2: 35266056A/G
1-2: 35266100T/G
1-2: 35266286C/T
1-2: 35266535T/A
1-2: 35271971T/G
1-2: 35308670T/C
1-2: 35466646C/A
1-2: 35473418T/C
1-2: 35482713C/G
1-2: 35559758T/C
1-2: 35678641T/A
1-2: 35694285T/A
1-2: 35724291A/G
1-2: 35795734T/C
1-2: 35827388C/T
1-2: 35832000T/A
1-2: 35893262G/A
1-2: 35931414G/T
1-2: 35951893T/G
1-2: 35954221C/G
1-2: 35954352T/C
1-2: 35954479C/A
1-2: 35954628A/C
1-2: 35966650A/G
1-2: 35977129C/T
1-2: 35977269C/G
1-2: 35995321G/A
1-2: 36034233T/C
1-2: 36035421C/T
1-2: 36089668A/G
1-2: 36205478C/T
1-2: 36206814C/T
1-2: 36229677A/G
1-2: 36309474T/C
1-2: 36441666T/C
1-2: 36500220T/C
1-2: 36671944C/G
1-2: 36705906C/T
1-2: 36845287G/C
1-2: 36967723G/C
1-2: 37236598T/G
1-2: 37327063C/T
1-2: 37361386G/C
1-2: 37418972A/T
1-2: 37480697C/T
1-2: 37573874T/A
1-2: 37671234T/C
1-2: 37716552T/G
1-2: 37822147C/T
1-2: 37835558C/T
1-2: 37976359A/G
1-2: 38092653T/C
1-2: 38306735C/T
1-2: 38399038C/T
1-2: 38591315T/A
1-2: 38593653C/A
1-2: 38629559A/G
1-2: 38657049A/G
1-2: 38715383T/C
1-2: 38807942T/C
1-2: 38885722C/T
1-2: 38897932C/T
1-2: 38941016T/G
1-2: 38952678T/C
1-2: 38972640C/T
1-2: 39026680G/A
1-2: 39063050T/C
1-2: 39076348C/T
1-2: 39087735T/C
1-2: 39167599A/G
1-2: 39189268A/G
1-2: 39236146G/A
1-2: 39339096G/T
1-2: 39365991G/A
1-2: 39406788A/G
1-2: 39442584G/A
1-2: 39442649G/A
1-2: 39564842C/T
1-2: 39606936T/C
1-2: 39625835C/T
1-2: 39658487T/A
1-2: 39686628T/C
1-2: 39699884G/A
1-2: 39714983C/A
1-2: 39717624C/A
1-2: 39749676G/A
1-2: 39787466C/T
1-2: 39804841C/T
1-2: 39804857A/G
1-2: 39804859T/G
1-2: 39805066T/C
1-2: 39805115C/T
1-2: 39805135T/C
1-2: 39805265G/A
1-2: 39805318C/G
1-2: 39805349C/G
1-2: 39805494C/.
1-2: 39811974T/C
1-2: 39886138T/A
1-2: 39923848T/A
1-2: 39924283A/G
1-2: 39974363A/G
1-2: 40028986C/T
1-2: 40138556G/T
1-2: 40163157A/G
1-2: 40269086T/C
1-2: 40295228T/G
1-2: 40314908T/C
1-2: 40372771G/A
1-2: 40428275G/T
1-2: 40481332G/A
1-2: 40576638C/A
1-2: 40633204A/T
1-2: 40660528A/G
1-2: 40661447G/T
1-2: 40661449T/A
1-2: 40739825A/C
1-2: 40752222G/A
1-2: 40864453G/A
1-2: 40874371A/C
1-2: 40946915A/G
1-2: 41016331G/T
1-2: 41018399G/C
1-2: 41019133T/C
1-2: 41019196A/G
1-2: 41138038G/A
1-2: 41148082G/A
1-2: 41192988A/G
1-2: 41327390T/C
1-2: 41431920C/T
1-2: 41490763G/A
1-2: 41490780G/A
1-2: 41490790T/A
1-2: 41688777C/T
1-2: 41725285T/C
1-2: 41782628A/G
1-2: 41792898G/A
1-2: 41824995C/.
1-2: 41825157T/A
1-2: 41884009C/T
1-2: 41923596G/T
1-2: 41943533A/C
1-2: 41996201T/C
1-2: 42027909A/G
1-2: 42046632G/A
1-2: 42057485C/T
1-2: 42116846G/T
1-2: 42165232T/A
1-2: 42245227A/G
1-2: 42281998A/C
1-2: 42292327C/A
1-2: 42293083G/A
1-2: 42336879C/T
1-2: 42349127G/T
1-2: 42353778A/C
1-2: 42354166A/G
1-2: 42363295A/C
1-2: 42363647C/T
1-2: 42363655G/A
1-2: 42381658A/T
1-2: 42396072A/G
1-2: 42408836G/A
1-2: 42420934G/T
1-2: 42456839G/T
1-2: 42473565A/T
1-2: 42486112A/G
1-2: 42622647A/G
1-2: 42760846C/T
1-2: 42771349C/T
1-2: 42785828A/T
1-2: 42786539T/G
1-2: 42789967C/T
1-2: 42790048C/T
1-2: 42791006T/C
1-2: 42798664A/G
1-2: 42799904A/G
1-2: 42815579A/G
1-2: 42838945T/C
1-2: 42847155A/G
1-2: 42852536G/A
1-2: 42929790T/C
1-2: 42952882C/T
1-2: 43087466C/T
1-2: 43173865C/T
1-2: 43186947C/T
1-2: 43219804A/C
1-2: 43299215G/A
1-2: 43359650A/G
1-2: 43386258C/T
1-2: 43414496G/T
1-2: 43436450T/G
1-2: 43478365T/A
1-2: 43501000T/C
1-2: 43512455G/A
1-2: 43524970T/C
1-2: 43560702T/C
1-2: 43591806C/A
1-2: 43608333T/C
1-2: 43639294T/C
1-2: 43677219C/G
1-2: 43709335G/A
1-2: 43709345A/G
1-2: 43793282C/T
1-2: 43807398C/T
1-2: 43855786A/C
1-2: 43891410G/A
1-2: 43915474T/C
1-2: 44037508A/G
1-2: 44129947T/C
1-2: 44217567A/T
1-2: 44384801G/A
1-2: 44579051G/A
1-2: 44622688C/A
1-2: 44645944T/C
1-2: 44712093T/G
1-2: 44748925A/C
1-2: 44793543C/G
1-2: 44841646G/A
1-2: 44888100C/T
1-2: 44954055C/A
1-2: 45008628A/C
1-2: 45063975T/G
1-2: 45142702C/A
1-2: 45162185C/G
1-2: 45176067T/C
1-2: 45209728C/T
1-2: 45236297A/G
1-2: 45278660A/C
1-2: 45326429C/T
1-2: 45433093A/G
1-2: 45447556C/T
1-2: 45476127C/T
1-2: 45505299A/G
1-2: 45583647A/C
1-2: 45620264A/T
1-2: 45637773C/A
1-2: 45650592A/G
1-2: 45650709T/C
1-2: 45664867T/G
1-2: 45665373T/C
1-2: 45665379A/G
1-2: 45677272C/A
1-2: 45690374C/A
1-2: 45735635C/T
1-2: 45795240T/G
1-2: 45833652C/G
1-2: 46084584G/T
1-2: 46084730C/T
1-2: 46090851T/C
1-2: 46103113C/T
1-2: 46123684G/A
1-2: 46152509C/G
1-2: 46193284A/G
1-2: 46231530T/G
1-2: 46257835T/C
1-2: 46340786A/G
1-2: 46372923G/T
1-2: 46389291T/C
1-2: 46531629T/C
1-2: 46579173C/G
1-2: 46595028G/T
1-2: 46646030T/C
1-2: 46724879G/A
1-2: 46725206C/T
1-2: 46727210G/A
1-2: 46727219T/C
1-2: 46727405T/C
1-2: 46753601C/T
1-2: 46756940A/T
1-2: 46758903G/C
1-2: 46759082C/G
1-2: 46759152A/G
1-2: 46759198C/T
1-2: 46765568C/G
1-2: 46779973C/G
1-2: 46805358G/A
1-2: 46849220A/G
1-2: 46897977T/A
1-2: 46922556A/G
1-2: 46929208T/A
1-2: 46942004G/C
1-2: 46979633A/C
1-2: 47030838C/A
1-2: 47122615T/G
1-2: 47179948G/C
1-2: 47188325G/C
1-2: 47279281C/A
1-2: 47358883C/T
1-2: 47390976T/C
1-2: 47487872T/A
1-2: 47536767T/C
1-2: 47569170A/G
1-2: 47634310A/G
1-2: 47813386G/A
1-2: 47860382T/C
1-2: 47878690C/A
1-2: 47930412G/T
1-2: 47954722A/G
1-2: 47978913A/G
1-2: 47999081G/A
1-2: 48012742C/G
1-2: 48023473T/C
1-2: 48065652C/T
1-2: 48087805C/T
1-2: 48099893T/C
1-2: 48149202G/A
1-2: 48160725A/C
1-2: 48173200G/T
1-2: 48187403T/C
1-2: 48195659C/.
1-2: 48216029C/T
1-2: 48235894T/G
1-2: 48308943A/G
1-2: 48328207A/G
1-2: 48413878C/T
1-2: 48467500T/A
1-2: 48494712T/A
1-2: 48494916T/A
1-2: 48517562A/G
1-2: 48517747C/T
1-2: 48518732G/A
1-2: 48528058A/T
1-2: 48552935C/A
1-2: 48595680A/T
1-2: 48668002A/G
1-2: 48749931A/G
1-2: 48799066A/G
1-2: 48830435A/G
1-2: 48983141T/C
1-2: 48994227A/T
1-2: 49004975C/T
1-2: 49038992T/C
1-2: 49049762A/C
1-2: 49053041C/T
1-2: 49134538T/A
1-2: 49217182C/G
1-2: 49268187G/A
1-2: 49351346G/A
1-2: 49417039C/T
1-2: 49502607A/G
1-2: 49520277T/G
1-2: 49556410T/G
1-2: 49585834C/T
1-2: 49611475G/A
1-2: 49672799G/A
1-2: 49740304T/G
1-2: 49799790G/A
1-2: 49873103A/G
1-2: 49943523A/G
1-2: 49976000C/T
1-2: 50029969A/G
1-2: 50114515A/T
1-2: 50161085G/A
1-2: 50291896C/T
1-2: 50331642A/C
1-2: 50377606A/C
1-2: 50418502G/A
1-2: 50450621A/G
1-2: 50479709A/G
1-2: 50572230T/A
1-2: 50713717G/T
1-2: 50814496G/A
1-2: 50820772T/A
1-2: 50874532G/A
1-2: 50908407T/C
1-2: 50953842C/T
1-2: 50981385T/C
1-2: 51060884T/A
1-2: 51095144C/A
1-2: 51262452C/T
1-2: 51379287A/G
1-2: 51390178G/A
1-2: 51400201G/A
1-2: 51439382T/G
1-2: 51475004A/C
1-2: 51508875C/T
1-2: 51591607G/A
1-2: 51698615T/C
1-2: 51712099C/T
1-2: 51723068T/C
1-2: 51733916G/A
1-2: 51758832T/C
1-2: 51771860A/G
1-2: 51891206T/C
1-2: 51976708T/C
1-2: 51978592T/A
1-2: 52074311C/T
1-2: 52141592G/A
1-2: 52199536T/C
1-2: 52235610A/G
1-2: 52246739G/T
1-2: 52291280T/A
1-2: 52326600T/C
1-2: 52347689T/C
1-2: 52393295G/A
1-2: 52406377C/A
1-2: 52455952A/G
1-2: 52492212G/C
1-2: 52653167A/C
1-2: 52693460G/T
1-2: 52693887T/G
1-2: 52693922C/T
1-2: 52705356G/A
1-2: 52752665T/A
1-2: 52752886G/A
1-2: 52763816A/G
1-2: 52812007A/C
1-2: 52823285A/C
1-2: 52839484T/G
1-2: 52887533A/G
1-2: 52905267A/T
1-2: 52906756C/T
1-2: 52972700G/A
1-2: 53151066A/G
1-2: 53216923A/T
1-2: 53260831A/G
1-2: 53272467G/T
1-2: 53323958G/A
1-2: 53396313T/A
1-2: 53449654C/T
1-2: 53511439G/A
1-2: 53643746C/T
1-2: 53643815G/C
1-2: 53714848C/T
1-2: 53795754G/A
1-2: 53866709C/A
1-2: 53909886A/T
1-2: 53952984G/T
1-2: 54016670T/A
1-2: 54033096C/T
1-2: 54064482G/A
1-2: 54127030C/T
1-2: 54172277C/T
1-2: 54190101C/A
1-2: 54233480A/G
1-2: 54305185G/A
1-2: 54364125C/G
1-2: 54364365T/G
1-2: 54390433G/A
1-2: 54443039A/T
1-2: 54471507G/A
1-2: 54509793T/A
1-2: 54522668T/C
1-2: 54523039A/T
1-2: 54523049G/A
1-2: 54659676C/T
1-2: 54708062C/T
1-2: 54736893G/T
1-2: 54798821G/C
1-2: 54900895C/T
1-2: 54918018G/C
1-2: 54929408C/T
1-2: 54955362G/A
1-2: 55012852C/A
1-2: 55054118A/T
1-2: 55103379C/A
1-2: 55210267C/G
1-2: 55239351G/A
1-2: 55317972A/G
1-2: 55361799C/T
1-2: 55393048C/T
1-2: 55423891A/G
1-2: 55470148A/G
1-2: 55529532G/A
1-2: 55558941C/T
1-2: 55593854G/A
1-2: 55620983G/T
1-2: 55643961A/G
1-2: 55674778A/G
1-2: 55690098T/C
1-2: 55779213C/T
1-2: 55812712A/G
1-2: 55855311G/A
1-2: 55916078C/T
1-2: 55944960A/T
1-2: 55999668A/G
1-2: 56074239G/A
1-2: 56107498C/T
1-2: 56144517G/A
1-2: 56181692T/A
1-2: 56198663T/C
1-2: 56237619G/A
1-2: 56262760T/A
1-2: 56325008A/C
1-2: 56360286C/T
1-2: 56417576T/G
1-2: 56504774G/A
1-2: 56522512A/G
1-2: 56617622C/A
1-2: 56618652A/G
1-2: 56725189C/T
1-2: 56826875A/G
1-2: 56840989A/G
1-2: 56860517A/G
1-2: 56934271A/G
1-2: 56980436A/G
1-2: 56985662T/C
1-2: 56987109T/C
1-2: 56987226G/A
1-2: 56991388T/A
1-2: 57010422G/C
1-2: 57094560C/T
1-2: 57213703A/G
1-2: 57279989A/C
1-2: 57301819A/C
1-2: 57394853A/T
1-2: 57409344T/A
1-2: 57424419G/A
1-2: 57440721C/G
1-2: 57519059G/A
1-2: 57581658A/T
1-2: 57593611G/C
1-2: 57686633G/C
1-2: 57721192T/G
1-2: 57847605A/G
1-2: 57922861C/G
1-2: 57968998T/C
1-2: 58041152A/T
1-2: 58074716A/T
1-2: 58115222G/C
1-2: 58149954A/C
1-2: 58368352T/A
1-2: 58527603G/A
1-2: 58585117A/G
1-2: 58631108A/T
1-2: 58677122T/A
1-2: 58806914A/G
1-2: 58897203T/G
1-2: 58976496G/A
1-2: 59041337T/C
1-2: 59052386C/T
1-2: 59091066G/A
1-2: 59109601C/T
1-2: 59140722T/A
1-2: 59181517T/C
1-2: 59276962T/C
1-2: 59278070G/C
1-2: 59278220T/C
1-2: 59280053A/C
1-2: 59366807A/C
1-2: 59398637A/G
1-2: 59582408T/C
1-2: 59585456G/A
1-2: 59609718A/G
1-2: 59643346G/A
1-2: 59645324T/C
1-2: 59651437C/T
1-2: 59698873G/C
1-2: 59888418A/G
1-2: 60014900C/G
1-2: 60156138A/G
1-2: 60225632T/C
1-2: 60318193C/T
1-2: 60374031A/G
1-2: 60524558A/G
1-2: 60551347A/G
1-2: 60551865G/A
1-2: 60551907A/T
1-2: 60753278T/C
1-2: 60902238G/A
1-2: 60967050T/C
1-2: 61067978G/A
1-2: 61164216A/G
1-2: 61270787G/C
1-2: 61330735A/G
1-2: 61354969C/T
1-2: 61609757G/A
1-2: 61671070A/G
1-2: 61780740G/A
1-2: 61979774G/C
1-2: 62016665A/T
1-2: 62043521T/C
1-2: 62048648G/A
1-2: 62049006A/G
1-2: 62113871C/T
1-2: 62190027T/A
1-2: 62237526C/G
1-2: 62302808T/C
1-2: 62377121C/A
1-2: 62427842G/A
1-2: 62605897T/A
1-2: 62639091G/A
1-2: 62738031C/G
1-2: 62794022T/G
1-2: 62810412A/G
1-2: 62859269C/G
1-2: 62885272C/T
1-2: 62934290A/G
1-2: 63012154G/A
1-2: 63067973A/C
1-2: 63100567C/T
1-2: 63102851T/G
1-2: 63142018G/A
1-2: 63228076A/G
1-2: 63229690A/T
1-2: 63240690A/G
1-2: 63243251T/C
1-2: 63244333A/G
1-2: 63245919T/C
1-2: 63251960T/A
1-2: 63295080G/A
1-2: 63305506A/G
1-2: 63334836A/T
1-2: 63372303A/G
1-2: 63442860A/G
1-2: 63474873C/T
1-2: 63520190A/G
1-2: 63623348T/A
1-2: 63659484C/T
1-2: 63763197G/A
1-2: 63903447C/G
1-2: 64064364G/A
1-2: 64121873T/A
1-2: 64150082T/A
1-2: 64218876T/C
1-2: 64242004G/A
1-2: 64242031T/C
1-2: 64243485A/C
1-2: 64251549G/A
1-2: 64289747G/A
1-2: 64327707A/T
1-2: 64388027A/C
1-2: 64440056G/A
1-2: 64484965G/A
1-2: 64499545G/A
1-2: 64571208A/G
1-2: 64621929T/C
1-2: 64633173C/T
1-2: 64644110G/A
1-2: 64645680T/C
1-2: 64646806A/G
1-2: 64660661T/G
1-2: 64730452A/G
1-2: 64806480C/T
1-2: 64995448T/A
1-2: 65028474A/G
1-2: 65106669T/A
1-2: 65126455G/A
1-2: 65214418C/T
1-2: 65244612A/T
1-2: 65248020C/T
1-2: 65249938C/T
1-2: 65250041C/A
1-2: 65256254C/T
1-2: 65260164T/A
1-2: 65260327T/G
1-2: 65313135T/C
1-2: 65370465T/A
1-2: 65385817A/G
1-2: 65427637G/T
1-2: 65471762G/A
1-2: 65510579C/T
1-2: 65635712T/G
1-2: 65664859T/C
1-2: 65668864C/G
1-2: 65668882A/G
1-2: 65668986C/G
1-2: 65669957C/T
1-2: 65706640A/G
1-2: 65718991C/T
1-2: 65729653A/C
1-2: 65730078G/A
1-2: 65750661T/G
1-2: 65750787A/T
1-2: 65751404C/T
1-2: 65751629A/T
1-2: 65751711T/C
1-2: 65757821T/C
1-2: 65758191T/C
1-2: 65758199C/T
1-2: 65758723G/A
1-2: 65760659T/C
1-2: 65761042G/C
1-2: 65761306T/A
1-2: 65780497A/C
1-2: 65780954T/C
1-2: 65791515C/G
1-2: 65937519T/C
1-2: 65977339T/G
1-2: 66058230C/G
1-2: 66072440G/A
1-2: 66090734C/G
1-2: 66174002T/G
1-2: 66256578A/G
1-2: 66328560A/T
1-2: 66427361C/T
1-2: 66454026G/A
1-2: 66669437T/C
1-2: 66746121A/T
1-2: 66804313T/C
1-2: 66838591C/T
1-2: 66879593C/G
1-2: 66920436G/A
1-2: 66960761A/C
1-2: 66994910A/G
1-2: 67075667C/A
1-2: 67097077A/G
1-2: 67206368C/T
1-2: 67244186T/C
1-2: 67323915C/G
1-2: 67374269G/A
1-2: 67408706G/A
1-2: 67467055A/G
1-2: 67467274A/G
1-2: 67468453G/A
1-2: 67468588T/C
1-2: 67507701G/C
1-2: 67534435G/A
1-2: 67546406C/T
1-2: 67671737A/T
1-2: 67671865A/T
1-2: 67712595T/A
1-2: 67757662T/C
1-2: 67778726G/T
1-2: 67918724T/A
1-2: 68009114T/A
1-2: 68116445C/T
1-2: 68137418C/T
1-2: 68144847A/C
1-2: 68178798C/T
1-2: 68211507G/A
1-2: 68233780A/G
1-2: 68235097G/A
1-2: 68235131C/T
1-2: 68235353T/G
1-2: 68245932A/G
1-2: 68256278A/G
1-2: 68357523C/T
1-2: 68441250T/C
1-2: 68502136G/A
1-2: 68512523C/A
1-2: 68622199A/G
1-2: 68623735C/T
1-2: 68633937C/T
1-2: 68694237C/T
1-2: 68741231G/C
1-2: 68773236T/C
1-2: 68799578G/A
1-2: 68824652C/T
1-2: 68854321T/C
1-2: 68894566A/C
1-2: 68917358T/C
1-2: 68930015G/A
1-2: 68963155T/G
1-2: 68973614G/A
1-2: 68981496T/C
1-2: 69000327C/A
1-2: 69016256T/A
1-2: 69037280G/A
1-2: 69077302A/G
1-2: 69106870C/A
1-2: 69169117A/G
1-2: 69248988A/G
1-2: 69350709G/A
1-2: 69394156G/A
1-2: 69420127C/A
1-2: 69437962A/T
1-2: 69472591C/T
1-2: 69507350T/C
1-2: 69531078G/C
1-2: 69541534T/G
1-2: 69554521G/C
1-2: 69614098C/T
1-2: 69668202A/C
1-2: 69694174T/C
1-2: 69771288T/G
1-2: 69852339T/G
1-2: 69882586T/C
1-2: 69889225G/A
1-2: 69889408T/C
1-2: 69891318G/A
1-2: 69893069G/A
1-2: 69921178T/C
1-2: 69992076A/G
1-2: 70064379C/T
1-2: 70089989T/C
1-2: 70136924G/A
1-2: 70172074T/C
1-2: 70241278G/A
1-2: 70254536T/G
1-2: 70265530C/T
1-2: 70359301G/A
1-2: 70426334C/T
1-2: 70496512G/A
1-2: 70527624C/T
1-2: 70587689A/G
1-2: 70625307G/T
1-2: 70654024T/C
1-2: 70728572C/A
1-2: 70807807C/A
1-2: 70818491T/C
1-2: 70826754A/T
1-2: 70826824T/A
1-2: 70838897G/A
1-2: 70935341A/T
1-2: 71029980T/G
1-2: 71071834C/T
1-2: 71133269A/G
1-2: 71182599C/T
1-2: 71242257C/T
1-2: 71242439A/G
1-2: 71266003G/T
1-2: 71278179C/T
1-2: 71292933A/G
1-2: 71299192C/G
1-2: 71299459G/A
1-2: 71309686C/A
1-2: 71332580T/A
1-2: 71463312G/T
1-2: 71467784A/G
1-2: 71487631C/T
1-2: 71540665C/T
1-2: 71600813T/C
1-2: 71707049T/C
1-2: 71856449G/T
1-2: 71876497C/T
1-2: 71891421T/C
1-2: 71913486G/C
1-2: 71999897A/T
1-2: 72119643G/A
1-2: 72121554C/T
1-2: 72166511A/C
1-2: 72314520T/C
1-2: 72377248G/T
1-2: 72706184A/G
1-2: 72845631G/C
1-2: 72949745G/A
1-2: 73063526T/C
1-2: 73113980G/T
1-2: 73124793G/C
1-2: 73216742A/G
1-2: 73257530G/A
1-2: 73484474C/T
1-2: 73604568G/A
1-2: 73828377A/T
1-2: 73855538A/G
1-2: 73953174A/T
1-2: 74024595A/G
1-2: 74060612T/C
1-2: 74290431C/A
1-2: 74482782A/G
1-2: 74501353C/T
1-2: 74513224G/A
1-2: 74570111T/G
1-2: 74633309T/G
1-2: 74855538G/T
1-2: 74946358C/T
1-2: 75130802T/A
1-2: 75143988A/G
1-2: 75539069A/T
1-2: 75597366G/A
1-2: 75798882A/G
1-2: 75806045C/T
1-2: 75950374T/C
1-2: 76060705G/A
1-2: 76078449C/T
1-2: 76166435T/C
1-2: 76237984T/C
1-2: 76267854A/C
1-2: 76447909T/C
1-2: 76506624A/T
1-2: 76565999C/T
1-2: 76684418C/T
1-2: 76711867C/T
1-2: 76738400C/T
1-2: 76921798A/T
1-2: 76924799G/A
1-2: 76988626C/T
1-2: 77053620C/T
1-2: 77159054C/T
1-2: 77588572G/C
1-2: 77607914A/G
1-2: 77619858G/T
1-2: 77674350A/T
1-2: 77846345A/G
1-2: 77939867T/A
1-2: 78025833T/C
1-2: 78056952T/C
1-2: 78129977A/G
1-2: 78269812A/G
1-2: 78282724A/C
1-2: 78545115A/T
1-2: 78674565C/T
1-2: 78693619A/G
1-2: 78709120C/T
1-2: 78850406C/T
1-2: 78874107C/T
1-2: 78883332T/A
1-2: 78901643A/C
1-2: 78924245C/T
1-2: 79127653G/A
1-2: 79264970T/A
1-2: 79533915C/T
1-3: 696483T/G
1-3: 1006048T/C
1-3: 1012063A/G
1-3: 1034776G/A
1-3: 1518869A/T
1-3: 1521087C/A
1-3: 1526504G/A
1-3: 1529292G/C
1-3: 1536448A/G
1-3: 1539910C/T
1-3: 1553491T/G
1-3: 1826979T/C
1-3: 1906308G/A
1-3: 2183754A/T
1-3: 2184635T/C
1-3: 2293932G/T
1-3: 2404748T/C
1-3: 2522842A/C
1-3: 2570665A/C
1-3: 2763584C/G
1-3: 2996516C/G
1-3: 3067001C/A
1-3: 3073642A/G
1-3: 3080493G/A
1-3: 3102303A/C
1-3: 3142800G/A
1-3: 3194875C/A
1-3: 3270311G/T
1-3: 3278431A/G
1-3: 3353065C/A
1-3: 3378809G/A
1-3: 3417915T/C
1-3: 3441230C/G
1-3: 3536962C/G
1-3: 3551114A/G
1-3: 3614700T/C
1-3: 3631373C/T
1-3: 3665560C/T
1-3: 3810555A/G
1-3: 3866129C/T
1-3: 3876185T/C
1-3: 3974319A/T
1-3: 3982507C/T
1-3: 4010362C/T
1-3: 4011331G/T
1-3: 4074915A/G
1-3: 4107919T/C
1-3: 4114503A/G
1-3: 4167760C/A
1-3: 4218620C/T
1-3: 4296365C/G
1-3: 4361102A/G
1-3: 4384938T/C
1-3: 4403394C/T
1-3: 4534700T/C
1-3: 4657637T/C
1-3: 4749574G/C
1-3: 4763949G/A
1-3: 4781033T/C
1-3: 4818077C/T
1-3: 4831846T/C
1-3: 4849754T/C
1-3: 4851071G/A
1-3: 4878313A/G
1-3: 4952138A/T
1-3: 5012037A/G
1-3: 5122925A/G
1-3: 5133485C/T
1-3: 5143755A/G
1-3: 5169645G/A
1-3: 5171089T/G
1-3: 5210051A/G
1-3: 5255565T/C
1-3: 5305173A/G
1-3: 5366167T/C
1-3: 5436965G/T
1-3: 5458669T/A
1-3: 5646252G/A
1-3: 5691221C/A
1-3: 5784105C/T
1-3: 5841846C/T
1-3: 5915566C/T
1-3: 5986711T/C
1-3: 6039113T/C
1-3: 6120201A/G
1-3: 6138807A/G
1-3: 6185371G/A
1-3: 6303486C/G
1-3: 6365615G/A
1-3: 6485896T/A
1-3: 6496308G/T
1-3: 6566560C/T
1-3: 6775275C/T
1-3: 6866592A/T
1-3: 6893267T/C
1-3: 6968819G/A
1-3: 7007318C/A
1-3: 7072738T/C
1-3: 7109676C/T
1-3: 7155054G/A
1-3: 7237260A/G
1-3: 7264764A/G
1-3: 7264889A/C
1-3: 7340480C/A
1-3: 7375536C/T
1-3: 7386408A/G
1-3: 7386975T/C
1-3: 7494465T/A
1-3: 7581683C/T
1-3: 7589277C/T
1-3: 7713868T/C
1-3: 7909511A/G
1-3: 7921206C/T
1-3: 7956990G/A
1-3: 8104290C/T
1-3: 8139379C/T
1-3: 8186676T/A
1-3: 8331845A/C
1-3: 8377055A/C
1-3: 8393372G/A
1-3: 8470690A/G
1-3: 8674958C/A
1-3: 8837473C/G
1-3: 8838242C/A
1-3: 8838729G/A
1-3: 8838767C/T
1-3: 8853092G/C
1-3: 8939032T/C
1-3: 8961442C/G
1-3: 8974772C/G
1-3: 9008426T/C
1-3: 9071547C/T
1-3: 9072363C/T
1-3: 9072713C/T
1-3: 9072882G/A
1-3: 9073226T/C
1-3: 9074028C/T
1-3: 9074348T/A
1-3: 9077892A/T
1-3: 9107410G/C
1-3: 9107493C/A
1-3: 9146630C/T
1-3: 9222481C/A
1-3: 9255969T/A
1-3: 9283852G/A
1-3: 9284159G/A
1-3: 9285805C/T
1-3: 9291422A/G
1-3: 9292726C/G
1-3: 9292873C/T
1-3: 9293447C/T
1-3: 9313141C/A
1-3: 9313237C/T
1-3: 9313659G/A
1-3: 9313683T/C
1-3: 9313928G/C
1-3: 9314235G/A
1-3: 9324363C/T
1-3: 9325484A/G
1-3: 9341282C/A
1-3: 9356575G/A
1-3: 9368355T/G
1-3: 9398586T/A
1-3: 9411867G/A
1-3: 9436249C/T
1-3: 9436523T/G
1-3: 9448086A/G
1-3: 9492852A/C
1-3: 9586890A/T
1-3: 9598312G/A
1-3: 9662223T/C
1-3: 9693632G/A
1-3: 9755701T/C
1-3: 9967274A/C
1-3: 10081574G/A
1-3: 10104155C/A
1-3: 10160486G/T
1-3: 10234726T/C
1-3: 10255966G/A
1-3: 10267797G/C
1-3: 10268510T/G
1-3: 10302415T/C
1-3: 10333053T/C
1-3: 10417689T/C
1-3: 10438348G/A
1-3: 10454399A/G
1-3: 10490451G/A
1-3: 10528568A/C
1-3: 10539394C/G
1-3: 10539932G/A
1-3: 10539997C/A
1-3: 10540054C/T
1-3: 10549141A/C
1-3: 10549558T/C
1-3: 10549725T/G
1-3: 10552784T/C
1-3: 10561916A/C
1-3: 10617537T/C
1-3: 10621145G/A
1-3: 10633548T/A
1-3: 10658114A/C
1-3: 10675762A/C
1-3: 10720663A/G
1-3: 10785573T/C
1-3: 10800181A/C
1-3: 10835265G/A
1-3: 10910800C/T
1-3: 10960928G/A
1-3: 10978252A/G
1-3: 11009224G/T
1-3: 11028270G/A
1-3: 11114603T/C
1-3: 11137408A/C
1-3: 11163929G/A
1-3: 11209779A/T
1-3: 11218052A/C
1-3: 11222231G/A
1-3: 11227766A/G
1-3: 11263842T/A
1-3: 11277171G/T
1-3: 11278226C/A
1-3: 11344663T/G
1-3: 11345640C/T
1-3: 11391953A/G
1-3: 11434230A/G
1-3: 11453089C/T
1-3: 11475994G/C
1-3: 11484957C/T
1-3: 11485120C/A
1-3: 11485351C/A
1-3: 11486929C/T
1-3: 11492130G/A
1-3: 11493470T/A
1-3: 11493573C/T
1-3: 11516834T/C
1-3: 11563582T/A
1-3: 11600135A/G
1-3: 11656635T/C
1-3: 11668365C/T
1-3: 11704167A/G
1-3: 11794858G/T
1-3: 11837837C/T
1-3: 11912265A/G
1-3: 11926880T/A
1-3: 11938910C/A
1-3: 11959549T/A
1-3: 11964481C/T
1-3: 12027316C/G
1-3: 12066932A/T
1-3: 12310718A/T
1-3: 12326439A/G
1-3: 12340783T/C
1-3: 12357134A/G
1-3: 12374069A/G
1-3: 12429992C/T
1-3: 12465628G/A
1-3: 12539726T/G
1-3: 12582184C/A
1-3: 12617183A/T
1-3: 12631123A/G
1-3: 12716933G/T
1-3: 12737005A/T
1-3: 12739072C/T
1-3: 12797286T/A
1-3: 12849795C/T
1-3: 12922343G/A
1-3: 12991454G/T
1-3: 13022697G/A
1-3: 13026230G/T
1-3: 13026236G/A
1-3: 13103569G/A
1-3: 13137754G/A
1-3: 13168850G/A
1-3: 13206040C/T
1-3: 13238472A/T
1-3: 13240579G/C
1-3: 13267695T/A
1-3: 13282920T/A
1-3: 13317999G/T
1-3: 13356723C/T
1-3: 13402376T/A
1-3: 13428624C/T
1-3: 13456819G/T
1-3: 13489285C/A
1-3: 13554983T/C
1-3: 13567100C/T
1-3: 13587462C/T
1-3: 13602740T/C
1-3: 13622272C/G
1-3: 13623936T/C
1-3: 13676564G/A
1-3: 13678005T/A
1-3: 13751786A/G
1-3: 13801259A/G
1-3: 13878249C/G
1-3: 13913912A/C
1-3: 13961054T/C
1-3: 14017592C/T
1-3: 14091743A/T
1-3: 14122038T/G
1-3: 14138613G/A
1-3: 14229180T/C
1-3: 14287091C/T
1-3: 14396650A/T
1-3: 14463774C/T
1-3: 14470544A/T
1-3: 14500280C/T
1-3: 14501432C/T
1-3: 14527170G/A
1-3: 14571105A/G
1-3: 14612263A/G
1-3: 14624707G/A
1-3: 14689573G/A
1-3: 14766968T/G
1-3: 14767013C/T
1-3: 14767160A/T
1-3: 14825601C/G
1-3: 14900045T/C
1-3: 14976163A/G
1-3: 15032193G/C
1-3: 15058199C/T
1-3: 15092943T/A
1-3: 15141584G/T
1-3: 15181731C/T
1-3: 15259126C/T
1-3: 15391660C/T
1-3: 15491325G/A
1-3: 15577716G/A
1-3: 15664268T/C
1-3: 15676721T/A
1-3: 15700291T/G
1-3: 15730889A/C
1-3: 15773371G/A
1-3: 15815399A/G
1-3: 15845560A/T
1-3: 15872290C/G
1-3: 15925514T/C
1-3: 16029870C/T
1-3: 16044233A/G
1-3: 16046009T/C
1-3: 16066330G/A
1-3: 16066748A/C
1-3: 16066813T/C
1-3: 16078730A/G
1-3: 16112769G/T
1-3: 16166675T/G
1-3: 16206146T/C
1-3: 16211897A/T
1-3: 16224900G/T
1-3: 16236262G/T
1-3: 16250733T/C
1-3: 16251033T/C
1-3: 16279370A/G
1-3: 16393377A/G
1-3: 16415887T/C
1-3: 16430870C/T
1-3: 16470707G/T
1-3: 16508505A/G
1-3: 16521807C/T
1-3: 16536440A/T
1-3: 16585037G/A
1-3: 16585709T/C
1-3: 16585769T/A
1-3: 16601085G/A
1-3: 16611119T/C
1-3: 16611271A/G
1-3: 16611436T/C
1-3: 16621559T/A
1-3: 16654278C/A
1-3: 16665453G/C
1-3: 16700264G/A
1-3: 16727375A/T
1-3: 16776664G/C
1-3: 16826597G/C
1-3: 16836602A/C
1-3: 16927998T/A
1-3: 16941419C/T
1-3: 17001907G/A
1-3: 17004068G/A
1-3: 17041968T/G
1-3: 17078180C/T
1-3: 17243351C/T
1-3: 17322704C/T
1-3: 17397124A/.
1-3: 17397126A/T
1-3: 17397132C/T
1-3: 17397176T/A
1-3: 17397179T/C
1-3: 17397187C/G
1-3: 17397207C/T
1-3: 17397348T/.
1-3: 17397354T/A
1-3: 17397356T/A
1-3: 17397363T/G
1-3: 17397402T/A
1-3: 17397420T/C
1-3: 17397519A/G
1-3: 17412128A/G
1-3: 17457185A/G
1-3: 17489520G/C
1-3: 17500201T/A
1-3: 17510939T/C
1-3: 17533472T/C
1-3: 17544282G/A
1-3: 17560267A/C
1-3: 17580356T/A
1-3: 17595579C/T
1-3: 17601417A/G
1-3: 17622442G/T
1-3: 17649130A/G
1-3: 17656886A/G
1-3: 17676147G/A
1-3: 17688236A/G
1-3: 17711044T/C
1-3: 17713147C/T
1-3: 17750221A/T
1-3: 17804652C/A
1-3: 17817164C/T
1-3: 17851403C/A
1-3: 17926138A/C
1-3: 17981571C/G
1-3: 18046614G/T
1-3: 18080718G/A
1-3: 18229183T/G
1-3: 18230705T/C
1-3: 18241675C/T
1-3: 18270421A/G
1-3: 18502891A/G
1-3: 18658607G/A
1-3: 18705867G/A
1-3: 18710692G/C
1-3: 18754209T/C
1-3: 18771594A/G
1-3: 18782753T/A
1-3: 18782951T/A
1-3: 18784110C/T
1-3: 18784448A/G
1-3: 18786054C/A
1-3: 18795730C/A
1-3: 18808720T/G
1-3: 18866523A/C
1-3: 18880045G/T
1-3: 18880348C/A
1-3: 18898813C/G
1-3: 18913092A/G
1-3: 19038055A/T
1-3: 19070152C/A
1-3: 19098556G/A
1-3: 19133865A/C
1-3: 19191321T/A
1-3: 19257937G/A
1-3: 19270474T/G
1-3: 19300761A/T
1-3: 19335150C/T
1-3: 19394933C/T
1-3: 19413521G/A
1-3: 19417916T/C
1-3: 19418542G/A
1-3: 19420604C/T
1-3: 19420705G/A
1-3: 19420811T/C
1-3: 19421170C/T
1-3: 19421318T/C
1-3: 19422093G/T
1-3: 19422694C/G
1-3: 19423010C/A
1-3: 19423362C/T
1-3: 19423412A/G
1-3: 19423476T/A
1-3: 19423513C/T
1-3: 19423631A/G
1-3: 19423846G/A
1-3: 19428650T/G
1-3: 19430593C/A
1-3: 19435912C/G
1-3: 19467622C/A
1-3: 19518360T/C
1-3: 19546823T/C
1-3: 19559769A/G
1-3: 19571022T/A
1-3: 19581907A/G
1-3: 19664935C/A
1-3: 19716591A/T
1-3: 19728242T/A
1-3: 19827048T/G
1-3: 19882009C/T
1-3: 19928815A/C
1-3: 19985721A/G
1-3: 20028149T/C
1-3: 20038160C/T
1-3: 20094654G/A
1-3: 20137862C/G
1-3: 20139611G/C
1-3: 20214673A/T
1-3: 20255847G/C
1-3: 20309594C/T
1-3: 20375227G/T
1-3: 20375903T/A
1-3: 20449600C/A
1-3: 20558572G/A
1-3: 20575727G/A
1-3: 20575911A/T
1-3: 20639631C/T
1-3: 20780643C/T
1-3: 20829167C/T
1-3: 20860988C/G
1-3: 20894404G/C
1-3: 20928590A/G
1-3: 20989898A/G
1-3: 20989919T/G
1-3: 21001719C/A
1-3: 21032674T/A
1-3: 21116938T/G
1-3: 21136060T/C
1-3: 21139643C/A
1-3: 21139762A/G
1-3: 21139771C/G
1-3: 21142644T/C
1-3: 21142812C/A
1-3: 21143033T/G
1-3: 21181852A/G
1-3: 21204623C/T
1-3: 21229814T/C
1-3: 21247053T/G
1-3: 21291260A/C
1-3: 21375790A/G
1-3: 21397167G/C
1-3: 21450366T/C
1-3: 21463635T/C
1-3: 21486879C/G
1-3: 21498398G/A
1-3: 21512756T/C
1-3: 21551816C/A
1-3: 21599590C/T
1-3: 21666987C/T
1-3: 21678629T/C
1-3: 21710598A/T
1-3: 21756334T/C
1-3: 21802194G/A
1-3: 21830896A/G
1-3: 21832551C/T
1-3: 21875623G/A
1-3: 21987277C/T
1-3: 22000174T/G
1-3: 22011228A/C
1-3: 22073900T/A
1-3: 22159480T/G
1-3: 22334530C/T
1-3: 22381925C/T
1-3: 22426530T/C
1-3: 22580626T/C
1-3: 22594793G/T
1-3: 22648619C/T
1-3: 22695219T/C
1-3: 22743647C/T
1-3: 22776926C/T
1-3: 22905604T/C
1-3: 22981667G/T
1-3: 23030792G/C
1-3: 23074391G/A
1-3: 23119813T/G
1-3: 23147652C/T
1-3: 23183849T/G
1-3: 23184660G/C
1-3: 23201380C/T
1-3: 23281197G/C
1-3: 23318053G/C
1-3: 23332270A/G
1-3: 23334310T/C
1-3: 23359587C/G
1-3: 23375382T/C
1-3: 23418801C/T
1-3: 23431488T/G
1-3: 23467631T/A
1-3: 23564110G/T
1-3: 23586116A/G
1-3: 23587719A/T
1-3: 23587732C/T
1-3: 23651884A/G
1-3: 23678371C/T
1-3: 23754504G/T
1-3: 23800780A/G
1-3: 23802032T/C
1-3: 23884812C/G
1-3: 23910348T/G
1-3: 23926130A/T
1-3: 23953817A/G
1-3: 24022996C/T
1-3: 24061121T/C
1-3: 24080983G/A
1-3: 24102907A/G
1-3: 24156033G/T
1-3: 24183547G/T
1-3: 24205876A/G
1-3: 24219696A/T
1-3: 24249967T/C
1-3: 24280079A/C
1-3: 24378558T/C
1-3: 24391666A/C
1-3: 24407995C/T
1-3: 24418562G/T
1-3: 24444650G/A
1-3: 24457060G/A
1-3: 24462201G/A
1-3: 24462305G/C
1-3: 24462516A/T
1-3: 24464556A/G
1-3: 24464818T/C
1-3: 24465548T/C
1-3: 24467126C/T
1-3: 24470981A/G
1-3: 24471118T/G
1-3: 24472172T/A
1-3: 24472489G/A
1-3: 24566880G/A
1-3: 24580826G/T
1-3: 24606581C/T
1-3: 24664948T/C
1-3: 24680856C/T
1-3: 24697370C/T
1-3: 24708236T/C
1-3: 24729832T/C
1-3: 24784316C/G
1-3: 24874763A/G
1-3: 24891649A/G
1-3: 24901742G/A
1-3: 24920111A/G
1-3: 24921172A/C
1-3: 25000241C/G
1-3: 25099956T/C
1-3: 25120960T/A
1-3: 25156683A/C
1-3: 25174741A/G
1-3: 25201036A/G
1-3: 25202607C/T
1-3: 25244316G/T
1-3: 25333839A/G
1-3: 25382175G/A
1-3: 25445130G/A
1-3: 25455202G/A
1-3: 25483221T/C
1-3: 25591761G/A
1-3: 25749288G/T
1-3: 25761787C/T
1-3: 25834141A/G
1-3: 25851017T/G
1-3: 25868278A/G
1-3: 25914034G/C
1-3: 25942933T/C
1-3: 25972290C/G
1-3: 25984150T/C
1-3: 26005448C/T
1-3: 26032945G/A
1-3: 26062197C/T
1-3: 26092628G/C
1-3: 26164281G/A
1-3: 26167003C/T
1-3: 26167118C/T
1-3: 26167246A/T
1-3: 26179002T/A
1-3: 26180647T/G
1-3: 26217718A/G
1-3: 26218874G/A
1-3: 26259676A/G
1-3: 26296283T/A
1-3: 26298909G/C
1-3: 26314793G/T
1-3: 26335166A/T
1-3: 26368152C/T
1-3: 26429725C/A
1-3: 26457359T/C
1-3: 26627756T/C
1-3: 26677927T/G
1-3: 26718215C/T
1-3: 26762635G/A
1-3: 26839150G/C
1-3: 26915633C/A
1-3: 27000315T/C
1-3: 27029577C/T
1-3: 27054209T/A
1-3: 27064030T/C
1-3: 27116374T/G
1-3: 27146669G/T
1-3: 27150686C/A
1-3: 27151022A/G
1-3: 27151037T/C
1-3: 27151050A/G
1-3: 27151071C/A
1-3: 27151162A/G
1-3: 27151193G/A
1-3: 27151274C/.
1-3: 27151352C/T
1-3: 27151658C/T
1-3: 27151667A/G
1-3: 27177144T/C
1-3: 27216897C/A
1-3: 27256902G/A
1-3: 27369711A/T
1-3: 27381554A/G
1-3: 27403677T/A
1-3: 27440423A/C
1-3: 27474591G/T
1-3: 27504796G/A
1-3: 27531687T/C
1-3: 27544212A/G
1-3: 27589988G/T
1-3: 27679670T/A
1-3: 27696751T/C
1-3: 27733146G/A
1-3: 27760562G/A
1-3: 27763018A/T
1-3: 27799601C/T
1-3: 27843364A/G
1-3: 27887096C/T
1-3: 27887107A/G
1-3: 27887442G/A
1-3: 27933935T/G
1-3: 28008021T/C
1-3: 28069104C/A
1-3: 28099621A/G
1-3: 28149400G/C
1-3: 28167299A/G
1-3: 28199547G/C
1-3: 28210220C/T
1-3: 28369567T/G
1-3: 28458082A/T
1-3: 28469779T/A
1-3: 28493015G/A
1-3: 28526781A/G
1-3: 28580535C/T
1-3: 28659008G/A
1-3: 28659725A/G
1-3: 28706900G/T
1-3: 28764919C/T
1-3: 28806948C/T
1-3: 28870132A/G
1-3: 28883612C/T
1-3: 28922079T/C
1-3: 28957244A/G
1-3: 29008567A/G
1-3: 29064049C/T
1-3: 29103103A/C
1-3: 29217984G/T
1-3: 29218025T/C
1-3: 29218042C/T
1-3: 29218045G/A
1-3: 29220698A/T
1-3: 29234402G/T
1-3: 29234596G/C
1-3: 29291600G/T
1-3: 29308652A/G
1-3: 29356339A/T
1-3: 29432231C/T
1-3: 29460687G/A
1-3: 29515432T/C
1-3: 29543073C/T
1-3: 29564912G/A
1-3: 29625976G/T
1-3: 29643337G/T
1-3: 29683125C/T
1-3: 29709977A/G
1-3: 29741401G/A
1-3: 29745985C/T
1-3: 29778688A/C
1-3: 29807820T/C
1-3: 29865381C/A
1-3: 29900868T/A
1-3: 29937546G/A
1-3: 29965486T/C
1-3: 29975599C/A
1-3: 30085343A/G
1-3: 30113028G/T
1-3: 30215178G/A
1-3: 30322286G/A
1-3: 30337173G/T
1-3: 30366522A/C
1-3: 30399079A/G
1-3: 30467435G/A
1-3: 30502664A/C
1-3: 30503003C/T
1-3: 30513250C/T
1-3: 30523495A/G
1-3: 30552935A/G
1-3: 30611206G/A
1-3: 30730282G/A
1-3: 30731730A/C
1-3: 30746648C/T
1-3: 30766383T/C
1-3: 30804303T/G
1-3: 30804835C/T
1-3: 30804841T/G
1-3: 30821215G/A
1-3: 30831633C/G
1-3: 30968786A/G
1-3: 31095405C/A
1-3: 31208729C/T
1-3: 31352333G/A
1-3: 31517421C/T
1-3: 31767636A/G
1-3: 31802172C/T
1-3: 31812343A/C
1-3: 31928850G/A
1-3: 32272456G/A
1-3: 32360940A/G
1-3: 32722602A/G
1-3: 32820109T/G
1-3: 32869189T/G
1-3: 32883261C/T
1-3: 32898484A/C
1-3: 33389547C/T
1-3: 33399866T/C
1-3: 33409943G/A
1-3: 33505605A/T
1-3: 33594296C/A
1-3: 33648055A/G
1-3: 33681454C/T
1-3: 33868863T/C
1-3: 33868892C/T
1-3: 33868927T/C
1-3: 33869654A/G
1-3: 33904979T/C
1-3: 33927529A/T
1-3: 33937693G/T
1-3: 33938260C/A
1-3: 33938401A/C
1-3: 33938429A/C
1-3: 34036933A/C
1-3: 34264619T/A
1-3: 34371930G/C
1-3: 34572520A/T
1-3: 34607425T/C
1-3: 34660177T/C
1-3: 34672657G/A
1-3: 34677391A/T
1-3: 34677703G/A
1-3: 34677785C/T
1-3: 34677904C/G
1-3: 34678207C/T
1-3: 34678230C/T
1-3: 34678316A/G
1-3: 34678556C/T
1-3: 34680870C/T
1-3: 34681101T/C
1-3: 34681315C/T
1-3: 34681548G/A
1-3: 34681745A/G
1-3: 34681944G/A
1-3: 34683077A/G
1-3: 34683395C/T
1-3: 34683943C/T
1-3: 34690487T/G
1-3: 34692772G/A
1-3: 34692911A/C
1-3: 34693324C/T
1-3: 34694554C/T
1-3: 34699525T/C
1-3: 34699937T/A
1-3: 34700107A/C
1-3: 34704123C/A
1-3: 34707796T/C
1-3: 34708072A/G
1-3: 34708194C/A
1-3: 34708337T/C
1-3: 34708690G/A
1-3: 34709005C/A
1-3: 34710184G/C
1-3: 34714428A/T
1-3: 34714605A/G
1-3: 34714659G/A
1-3: 34715183T/G
1-3: 34717786A/T
1-3: 34720792G/A
1-3: 34723059A/G
1-3: 34724813C/G
1-3: 34725120C/T
1-3: 34725319A/T
1-3: 34725795G/T
1-3: 34727612T/C
1-3: 34728525T/A
1-3: 34729861G/A
1-3: 34737076T/C
1-3: 34738015C/A
1-3: 34738936T/G
1-3: 34739077C/T
1-3: 34748286T/A
1-3: 34752105G/T
1-3: 34779389G/A
1-3: 34916854A/G
1-3: 35018580T/C
1-3: 35056462A/T
1-3: 35081622T/A
1-3: 35093189T/C
1-3: 35190833C/A
1-3: 35209524A/C
1-3: 35217965C/G
1-3: 35227969C/A
1-3: 35260970T/C
1-3: 35272390C/T
1-3: 35298551C/T
1-3: 35310977C/G
1-3: 35322767C/T
1-3: 35334938C/A
1-3: 35370420T/C
1-3: 35402504A/T
1-3: 35437462T/C
1-3: 35514608C/T
1-3: 35585192C/T
1-3: 35604441G/C
1-3: 35630760T/A
1-3: 35740549C/T
1-3: 35914811T/C
1-3: 36130485C/A
1-3: 36144619T/C
1-3: 36202042T/C
1-3: 36212591G/T
1-3: 36299859G/T
1-3: 36330251A/G
1-3: 36392074T/A
1-3: 36487940A/G
1-3: 36513070C/T
1-3: 36528604G/A
1-3: 36700705C/T
1-3: 36755201A/G
1-3: 37016598A/G
1-3: 37050842T/C
1-3: 37093961G/T
1-3: 37273613T/G
1-3: 37276781T/C
1-3: 37346035G/A
1-3: 37414892A/G
1-3: 37414904T/A
1-3: 37484891C/A
1-3: 37485181A/G
1-3: 37485421T/C
1-3: 37537475G/C
1-3: 37574592T/C
1-3: 37587433G/A
1-3: 37629743T/C
1-3: 37753555A/G
1-3: 37792816C/T
1-3: 37897534C/T
1-3: 38030391G/A
1-3: 38116822T/C
1-3: 38137579C/A
1-3: 38390111T/A
1-3: 38400904C/G
1-3: 38401967A/G
1-3: 38402443A/T
1-3: 38405701G/A
1-3: 38405821G/C
1-3: 38405948T/C
1-3: 38410404G/A
1-3: 38410559T/C
1-3: 38417407T/C
1-3: 38507202A/G
1-3: 38529310G/A
1-3: 38571878T/C
1-3: 38582251G/A
1-3: 38582714T/A
1-3: 38583348A/G
1-3: 38594537T/A
1-3: 38651101T/A
1-3: 38658406G/T
1-3: 38707328A/G
1-3: 38736381T/C
1-3: 38778971C/G
1-3: 38847128C/A
1-3: 38883494C/T
1-3: 38959339G/A
1-3: 39007848G/C
1-3: 39021980C/T
1-3: 39021998T/C
1-3: 39022188A/G
1-3: 39022196G/A
1-3: 39022205T/C
1-3: 39022243A/G
1-3: 39022254C/T
1-3: 39022275A/G
1-3: 39022293T/A
1-3: 39022304G/A
1-3: 39022314G/A
1-3: 39022428T/C
1-3: 39022431G/A
1-3: 39022499A/C
1-3: 39022501G/A
1-3: 39022502C/T
1-3: 39074568T/C
1-3: 39124284C/T
1-3: 39272268T/C
1-3: 39314546C/T
1-3: 39348778C/A
1-3: 39349399A/C
1-3: 39349505T/C
1-3: 39349706G/A
1-3: 39350590T/A
1-3: 39350698C/A
1-3: 39350991G/T
1-3: 39350993G/A
1-3: 39357490G/A
1-3: 39359403T/C
1-3: 39364530A/T
1-3: 39392147T/A
1-3: 39406489T/A
1-3: 39420209G/A
1-3: 39432216A/G
1-3: 39433033C/T
1-3: 39434891A/G
1-3: 39436804A/T
1-3: 39448264C/A
1-3: 39577806A/G
1-3: 39578206C/T
1-3: 39617774C/T
1-3: 39845904T/G
1-3: 39971365A/T
1-3: 39986667T/C
1-3: 40003265C/G
1-3: 40046043A/G
1-3: 40106181T/G
1-3: 40161002A/T
1-3: 40194508G/T
1-3: 40304670C/T
1-3: 40308979G/A
1-3: 40340084A/G
1-3: 40366760G/T
1-3: 40468709A/G
1-3: 40478440T/C
1-3: 40652122C/A
1-3: 40678976T/G
1-3: 40833774C/T
1-3: 40846598A/T
1-3: 40847875A/T
1-3: 40860447C/A
1-3: 40920998G/T
1-3: 40949360A/C
1-3: 40981549A/G
1-3: 41061958T/C
1-3: 41151690T/C
1-3: 41181738A/G
1-3: 41245071T/A
1-3: 41287636C/A
1-3: 41331384T/C
1-3: 41382136A/T
1-3: 41438933C/T
1-3: 41554244C/A
1-3: 41791161T/A
1-3: 41811862A/G
1-3: 41839361G/A
1-3: 41841408A/G
1-3: 41867103G/C
1-3: 41899055A/T
1-3: 41932116A/T
1-3: 41936020C/A
1-3: 41936584G/A
1-3: 41975516A/G
1-3: 42009015A/C
1-3: 42056839C/T
1-3: 42056864T/C
1-3: 42129980G/A
1-3: 42178930G/C
1-3: 42243635A/T
1-3: 42285543C/T
1-3: 42342789C/T
1-3: 42368424T/C
1-3: 42418679G/A
1-3: 42438571A/T
1-3: 42518620T/C
1-3: 42563566A/G
1-3: 42629599G/A
1-3: 42709989A/G
1-3: 42798505T/G
1-3: 42839730A/G
1-3: 42874435C/A
1-3: 42906968G/C
1-3: 42983588T/C
1-3: 43139080A/G
1-3: 43188091A/G
1-3: 43192272G/T
1-3: 43204810C/T
1-3: 43295485T/G
1-3: 43329944C/T
1-3: 43331459C/T
1-3: 43346323A/G
1-3: 43346448A/G
1-3: 43395832G/A
1-3: 43397579C/T
1-3: 43412018C/A
1-3: 43423117G/A
1-3: 43455105T/C
1-3: 43516909A/G
1-3: 43546427G/A
1-3: 43548089G/A
1-3: 43627874G/A
1-3: 43673030A/T
1-3: 43692279G/C
1-3: 43710052T/G
1-3: 43720365G/C
1-3: 43765092G/C
1-3: 43767280G/A
1-3: 43771638A/T
1-3: 43771642C/T
1-3: 43772521G/A
1-3: 43773712A/C
1-3: 43774917T/A
1-3: 43777258C/A
1-3: 43792267G/A
1-3: 43828831T/C
1-3: 43871775G/A
1-3: 43874394C/G
1-3: 43922896T/C
1-3: 43984222G/A
1-3: 44019643G/A
1-3: 44085976G/C
1-3: 44122548G/A
1-3: 44129241G/A
1-3: 44155158C/G
1-3: 44181989G/T
1-3: 44228467G/A
1-3: 44267891A/G
1-3: 44295261G/A
1-3: 44323707A/C
1-3: 44344347G/A
1-3: 44377037A/T
1-3: 44391386C/T
1-3: 44577014G/A
1-3: 44633740T/C
1-3: 44660511G/A
1-3: 44673270A/G
1-3: 44707218C/T
1-3: 44850968C/T
1-3: 44889675T/A
1-3: 44922150A/G
1-3: 44954723T/C
1-3: 44983458A/T
1-3: 44993642A/G
1-3: 45022782G/A
1-3: 45022888C/T
1-3: 45023026T/G
1-3: 45023431C/T
1-3: 45034738A/G
1-3: 45052516C/T
1-3: 45055945C/T
1-3: 45056016C/T
1-3: 45058505G/T
1-3: 45115815G/A
1-3: 45135948G/T
1-3: 45223345T/C
1-3: 45254143G/A
1-3: 45279329A/G
1-3: 45339506G/A
1-3: 45352510C/T
1-3: 45404829C/T
1-3: 45460705C/G
1-3: 45549724A/G
1-3: 45652850T/C
1-3: 45670198G/T
1-3: 45720846C/T
1-3: 45744134A/C
1-3: 45834618A/G
1-3: 45873099G/A
1-3: 45898806C/T
1-3: 45898816T/A
1-3: 45990563A/C
1-3: 46018421C/T
1-3: 46116286C/A
1-3: 46236331T/C
1-3: 46251717C/T
1-3: 46293651A/T
1-3: 46367934T/A
1-3: 46386234G/T
1-3: 46485077C/T
1-3: 46544777C/T
1-3: 46555563T/C
1-3: 46596778T/C
1-3: 46635903A/G
1-3: 46675354T/C
1-3: 46719004C/T
1-3: 46731348A/G
1-3: 46764078A/T
1-3: 46792683C/T
1-3: 46830013A/C
1-3: 46863138C/A
1-3: 46931020C/T
1-3: 47080611A/G
1-3: 47089573T/C
1-3: 47124314A/G
1-3: 47180493C/T
1-3: 47208829G/A
1-3: 47265784G/C
1-3: 47276486A/C
1-3: 47440268G/A
1-3: 47452554C/T
1-3: 47464540A/C
1-3: 47497576C/A
1-3: 47542628G/T
1-3: 47570376G/A
1-3: 47590934A/G
1-3: 47596645T/A
1-3: 47603156G/A
1-3: 47659310T/A
1-3: 47709358G/A
1-3: 47759943C/T
1-3: 47827651C/A
1-3: 47886637C/T
1-3: 47960362T/G
1-3: 47990302A/G
1-3: 48025855C/T
1-3: 48041170A/C
1-3: 48049856G/A
1-3: 48069395G/T
1-3: 48097126G/A
1-3: 48117016G/A
1-3: 48120679A/T
1-3: 48143502C/T
1-3: 48155163C/G
1-3: 48232768A/G
1-3: 48261876G/A
1-3: 48350752T/C
1-3: 48463056G/C
1-3: 48475016A/G
1-3: 48498081C/T
1-3: 48516780T/C
1-3: 48526444C/T
1-3: 48560839T/A
1-3: 48593987C/T
1-3: 48629535G/A
1-3: 48633394C/T
1-3: 48634535C/A
1-3: 48719469G/A
1-3: 48818524A/C
1-3: 48824959A/G
1-3: 48831401G/A
1-3: 48858247A/T
1-3: 48875703T/C
1-3: 48905270T/A
1-3: 48933000T/A
1-3: 49009898A/G
1-3: 49119536G/T
1-3: 49176820C/G
1-3: 49273364T/C
1-3: 49302756A/C
1-3: 49314185A/G
1-3: 49463290T/A
1-3: 49506288G/C
1-3: 49539237C/T
1-3: 49567371T/C
1-3: 49706246A/G
1-3: 49710845A/G
1-3: 49737117A/G
1-3: 49780013T/C
1-3: 49810794T/G
1-3: 49841790A/G
1-3: 49896761A/G
1-3: 49936897T/G
1-3: 50021883T/A
1-3: 50106374T/A
1-3: 50139827A/C
1-3: 50171427T/A
1-3: 50198414A/C
1-3: 50287007G/A
1-3: 50341708C/T
1-3: 50377086G/A
1-3: 50390200T/G
1-3: 50451963C/T
1-3: 50476447A/T
1-3: 50487081T/C
1-3: 50502252C/T
1-3: 50522060G/A
1-3: 50532863T/C
1-3: 50571523C/T
1-3: 50571791A/G
1-3: 50610309G/A
1-3: 50642357C/G
1-3: 50681579C/T
1-3: 50713308C/A
1-3: 50735246T/C
1-3: 50829253G/A
1-3: 50829620G/A
1-3: 50843022A/T
1-3: 50879950A/G
1-3: 50966796A/G
1-3: 51027258A/G
1-3: 51040056C/A
1-3: 51040480C/A
1-3: 51103810C/T
1-3: 51176339C/T
1-3: 51196808A/C
1-3: 51236364G/C
1-3: 51284685C/T
1-3: 51322427G/A
1-3: 51339353T/A
1-3: 51366239C/T
1-3: 51452325T/C
1-3: 51467321G/A
1-3: 51469725T/A
1-3: 51480942A/G
1-3: 51501966C/T
1-3: 51505755C/A
1-3: 51519880C/A
1-3: 51620137G/A
1-3: 51645096T/A
1-3: 51646519T/C
1-3: 51665527C/A
1-3: 51864861G/A
1-3: 51893630T/C
1-3: 51948871G/T
1-3: 52021303C/A
1-3: 52052960T/G
1-3: 52063587T/A
1-3: 52101114G/A
1-3: 52151563T/C
1-3: 52162071A/G
1-3: 52194555C/A
1-3: 52216523C/T
1-3: 52300073C/T
1-3: 52326847C/T
1-3: 52415066T/C
1-3: 52471908T/C
1-3: 52497506T/C
1-3: 52547773A/T
1-3: 52634483T/A
1-3: 52645796T/A
1-3: 52770674G/A
1-3: 52782991C/T
1-3: 52794734T/C
1-3: 52798141A/C
1-3: 52894469C/A
1-3: 52922514A/C
1-3: 52961091T/A
1-3: 53026531T/C
1-3: 53050915G/A
1-3: 53136334G/T
1-3: 53161937A/T
1-3: 53196764T/C
1-3: 53200153G/A
1-3: 53265396T/A
1-3: 53461137C/A
1-3: 53511087A/G
1-3: 53572074A/T
1-3: 53607485C/G
1-3: 53676594T/C
1-3: 53703450T/G
1-3: 53742167G/A
1-3: 53806780G/T
1-3: 53844472T/G
1-3: 53894241G/A
1-3: 54020381C/T
1-3: 54022492A/T
1-3: 54035416A/C
1-3: 54045903C/T
1-3: 54096904T/C
1-3: 54177593C/G
1-3: 54291891T/C
1-3: 54309554T/C
1-3: 54327514G/A
1-3: 54327637T/A
1-3: 54356288G/T
1-3: 54402843A/C
1-3: 54425868C/T
1-3: 54468988G/C
1-3: 54541388C/T
1-3: 54590694T/A
1-3: 54717288C/T
1-3: 54782129A/G
1-3: 54831749A/T
1-3: 54859079T/A
1-3: 54888501A/G
1-3: 54934415T/C
1-3: 54998381T/A
1-3: 55025912G/A
1-3: 55057598C/G
1-3: 55063714C/A
1-3: 55159147T/C
1-3: 55191675A/G
1-3: 55226951A/G
1-3: 55270489G/T
1-3: 55365832A/G
1-3: 55427268A/G
1-3: 55442002T/A
1-3: 55442519C/A
1-3: 55477710G/A
1-3: 55488631G/A
1-3: 55489146A/G
1-3: 55508256A/G
1-3: 55538600G/T
1-3: 55569996T/A
1-3: 55631853A/T
1-3: 55729442T/A
1-3: 55743446A/T
1-3: 55788672A/G
1-3: 55814901G/A
1-3: 55850115C/T
1-3: 55888282A/T
1-3: 55956339G/T
1-3: 55997381C/A
1-3: 56122640C/T
1-3: 56130868A/G
1-3: 56156641A/G
1-3: 56181782C/G
1-3: 56181808C/T
1-3: 56188818C/A
1-3: 56327705T/G
1-3: 56361484G/T
1-3: 56457800G/A
1-3: 56471817T/C
1-3: 56496116C/G
1-3: 56621107A/G
1-3: 56646608T/G
1-3: 56848871A/G
1-3: 56867799C/T
1-3: 56923639G/A
1-3: 56985134T/A
1-3: 57031977A/T
1-3: 57115710T/C
1-3: 57332261A/G
1-3: 57356741A/G
1-3: 57412253G/C
1-3: 57475009C/T
1-3: 57594414C/T
1-3: 57619730A/G
1-3: 57640006A/G
1-3: 57794019G/A
1-3: 57879831A/G
1-3: 57989096A/G
1-3: 58035426A/G
1-3: 58078227A/G
1-3: 58103870T/C
1-3: 58114840A/G
1-3: 58179056T/C
1-3: 58198721C/G
1-3: 58238421C/T
1-3: 58288645A/T
1-3: 58353260T/C
1-3: 58410483A/C
1-3: 58491980G/.
1-3: 58504296C/T
1-3: 58584256C/T
1-3: 58714123C/T
1-3: 58914464C/T
1-3: 58930754G/A
1-3: 58989502T/G
1-3: 59105263T/C
1-3: 59151293A/G
1-3: 59151323A/G
1-3: 59203094A/C
1-3: 59352674C/T
1-3: 59352700T/C
1-3: 59354967T/C
1-3: 59367778G/A
1-3: 59377832T/A
1-3: 59393265G/A
1-3: 59438247C/T
1-3: 59471610C/T
1-3: 59496205G/T
1-3: 59498738A/C
1-3: 59499116T/G
1-3: 59499807C/A
1-3: 59520664C/T
1-3: 59657725T/G
1-3: 59723958G/A
1-3: 59732864A/G
1-3: 59733479C/T
1-3: 59735414A/C
1-3: 59735518G/A
1-3: 59735965C/T
1-3: 59737306A/C
1-3: 59751845G/T
1-3: 59783320G/A
1-3: 59831598G/T
1-3: 59863565A/C
1-3: 59907385A/T
1-3: 59919634A/G
1-3: 59945675G/A
1-3: 59948343A/C
1-3: 59949993T/A
1-3: 59981826T/G
1-3: 59993925C/T
1-3: 60006271A/G
1-3: 60016181C/A
1-3: 60017044A/T
1-3: 60020220C/T
1-3: 60022942T/C
1-3: 60038059A/C
1-3: 60068272C/G
1-3: 60069520G/A
1-3: 60071534C/A
1-3: 60083194A/G
1-3: 60090554A/C
1-3: 60090686G/A
1-3: 60106820T/G
1-3: 60194661A/G
1-3: 60254072C/T
1-3: 60272819G/A
1-3: 60296323T/G
1-3: 60307418C/T
1-3: 60352107A/G
1-3: 60392341C/G
1-3: 60429203A/G
1-3: 60462401C/A
1-3: 60529991G/A
1-3: 60570398C/G
1-3: 60583957A/G
1-3: 60623892G/A
1-3: 60649892T/C
1-3: 60677861C/T
1-3: 60687989A/G
1-3: 60720264A/G
1-3: 60781883A/G
1-3: 60851875T/C
1-3: 60867047G/A
1-3: 60880075G/A
1-3: 60907783C/T
1-3: 60944821A/G
1-3: 60997985C/T
1-3: 61046644T/A
1-3: 61124004G/A
1-3: 61169960T/A
1-3: 61199587G/A
1-3: 61212106T/C
1-3: 61238659G/T
1-3: 61278460C/A
1-3: 61324622G/T
1-3: 61354598C/A
1-3: 61456917T/C
1-3: 61482383A/T
1-3: 61524770C/T
1-3: 61602080A/C
1-3: 61697831C/G
1-3: 61729539A/T
1-3: 61757999C/A
1-3: 61772544G/T
1-3: 61870736G/C
1-3: 62007572T/A
1-3: 62109006G/A
1-3: 62128172T/C
1-3: 62152883T/C
1-3: 62184711T/A
1-3: 62298670C/T
1-3: 62488948T/A
1-3: 62542306T/C
1-3: 62699507T/C
1-3: 62801715G/A
1-3: 62802015A/T
1-3: 62813087T/G
1-3: 62867868G/A
1-3: 62896569A/T
1-3: 62913678C/T
1-3: 63020869A/T
1-3: 63040289C/T
1-3: 63094824A/G
1-3: 63175288G/T
1-3: 63282231C/T
1-3: 63367138C/T
1-3: 63449159T/G
1-3: 63503354T/C
1-3: 63600292A/G
1-3: 63601979C/T
1-3: 63601985C/T
1-3: 63604978C/T
1-3: 63605143A/T
1-3: 63617648G/T
1-3: 63621923T/A
1-3: 63634381T/C
1-3: 63748245G/A
1-3: 63833560C/T
1-3: 63908976T/G
1-3: 63994349C/T
1-3: 64067744T/C
1-3: 64142414C/A
1-3: 64182654A/G
1-3: 64370572G/A
1-3: 64394567A/T
1-3: 64443910T/C
1-3: 64547152G/A
1-3: 64718496G/A
1-3: 64719835G/A
1-3: 64923639G/A
1-3: 65043581T/C
1-3: 65068131A/C
1-3: 65099598A/G
1-3: 65171804C/T
1-3: 65188206C/T
1-3: 65280784A/C
1-3: 65383981T/C
1-3: 65396140C/T
1-3: 65406832C/G
1-3: 65423033G/A
1-3: 65480375G/T
1-3: 65544169G/A
1-3: 65624606A/G
1-3: 65640651A/G
1-3: 65674451G/A
1-3: 65687291C/A
1-3: 65709712A/T
1-3: 65710329C/T
1-3: 65717222A/G
1-3: 65729944G/A
1-3: 65737141G/A
1-3: 65742495A/G
1-3: 65743199A/G
1-3: 65753385T/C
1-3: 65766066G/A
1-3: 65806372A/G
1-3: 65828740C/T
1-3: 65877610G/A
1-3: 65907219A/C
1-3: 65981005G/A
1-3: 66026271C/T
1-3: 66062573G/T
1-3: 66131200C/T
1-3: 66177642C/T
1-3: 66228956A/G
1-3: 66289471A/G
1-3: 66351428C/T
1-3: 66459814C/G
1-3: 66475387C/A
1-3: 66516903G/A
1-3: 66550150A/G
1-3: 66585140T/C
1-3: 66618720T/A
1-3: 66647661T/C
1-3: 66675733C/T
1-3: 66676208A/G
1-3: 66720727T/C
1-3: 66763740C/T
1-3: 66834257T/C
1-3: 66863482C/T
1-3: 66956257T/C
1-3: 66958327T/C
1-3: 66970449G/A
1-3: 67113618C/T
1-3: 67157545G/A
1-3: 67219616T/G
1-3: 67264535T/C
1-3: 67301234C/T
1-3: 67339003G/A
1-3: 67358373A/G
1-3: 67463848C/A
1-3: 67515556A/C
1-3: 67568664T/A
1-3: 67617573T/A
1-3: 67638056G/C
1-3: 67669584G/A
1-3: 67762187C/A
1-3: 67836107G/T
1-3: 67938383G/A
1-3: 67942355G/A
1-3: 67942865A/G
1-3: 67948448T/C
1-3: 67948832G/A
1-3: 67948955G/A
1-3: 67949513A/T
1-3: 67959370G/A
1-3: 67968389A/C
1-3: 67986590A/G
1-3: 67987525T/A
1-3: 67997965A/G
1-3: 68011150C/T
1-3: 68121740G/C
1-3: 68123052C/T
1-3: 68152516C/G
1-3: 68171687C/T
1-3: 68260243C/G
1-3: 68541076G/A
1-3: 68543152C/T
1-3: 68613263G/A
1-3: 68696397G/A
1-3: 68751846C/T
1-3: 68781245C/A
1-3: 68781752T/C
1-3: 68814861T/A
1-3: 68902417T/G
1-3: 68930298G/C
1-3: 68933533T/C
1-3: 68956619T/G
1-3: 68964349G/.
1-3: 68983209G/T
1-3: 69085203C/T
1-3: 69139091G/C
1-3: 69241058A/G
1-3: 69354709T/G
1-3: 69423626T/C
1-3: 69477971T/C
1-3: 69502107T/C
1-3: 69529002C/T
1-3: 69622959A/G
1-3: 69655399C/T
1-3: 69681390G/A
1-3: 69733721T/C
1-3: 69738902G/A
1-3: 69739004A/C
1-3: 69739105C/T
1-3: 69742967A/G
1-3: 69743077A/C
1-3: 69743393C/A
1-3: 69743448C/T
1-3: 69743498A/T
1-3: 69743689C/T
1-3: 69744503C/T
1-3: 69745032T/C
1-3: 69745065G/C
1-3: 69745824G/T
1-3: 69746150T/A
1-3: 69746277G/A
1-3: 69746570C/T
1-3: 69746676C/T
1-3: 69747272T/A
1-3: 69747277G/A
1-3: 69748736G/T
1-3: 69748873A/G
1-3: 69749252T/C
1-3: 69749363T/C
1-3: 69755590A/T
1-3: 69803562T/G
1-3: 69832674T/C
1-3: 69832979G/C
1-3: 69844288T/G
1-3: 69952634A/G
1-3: 69984913G/A
1-3: 69998608G/A
1-3: 70029381G/A
1-3: 70160817C/T
1-3: 70330311C/T
1-3: 70346801T/A
1-3: 70372306C/G
1-3: 70398371G/A
1-3: 70446857A/G
1-3: 70476968A/G
1-3: 70560983A/T
1-3: 70742167A/C
1-3: 70782344A/T
1-3: 70850934T/A
1-3: 70976021T/A
1-3: 71004864G/C
1-3: 71198633G/T
1-3: 71285748A/G
1-3: 71430646C/T
1-3: 71442633G/A
1-3: 71511048G/A
1-3: 71641738A/G
1-3: 71691022C/T
1-3: 71704407C/T
1-3: 71781376A/G
1-3: 71953998A/G
1-3: 71986748G/A
1-3: 72056109A/G
1-3: 72137506A/C
1-3: 72157735A/T
1-3: 72193007T/C
1-3: 72204778C/T
1-3: 72414897G/A
1-3: 72449586A/G
1-3: 72553161A/C
1-3: 72818625G/A
1-3: 72908866G/A
1-3: 73086306C/T
1-3: 73175374A/G
1-3: 73204370A/C
1-3: 73246726C/T
1-3: 73282179A/G
1-3: 73306077C/T
1-3: 73404386C/T
1-3: 73423896C/T
1-3: 73785992T/C
1-3: 73905581A/C
1-3: 73908772A/G
1-3: 73928665A/G
1-3: 73963206G/A
1-3: 74155397G/A
1-3: 74408568G/A
1-3: 74533064A/C
1-3: 74637529A/T
1-3: 75126184G/A
1-3: 75394509A/G
1-3: 75397355G/T
1-3: 75407425T/C
1-3: 75498833G/A
1-3: 75540973T/C
1-3: 75632348G/A
1-3: 76016646T/C
1-3: 76177630T/C
1-3: 76196408T/G
1-3: 76205047T/C
1-3: 76328943T/A
1-3: 76475594G/A
1-3: 76476933T/C
1-3: 76504449A/G
1-3: 76712077A/G
1-3: 76714590C/T
1-3: 76733667C/A
1-3: 76787039T/C
1-3: 77049339G/A
1-4: 120683T/A
1-4: 296646T/C
1-4: 814704G/T
1-4: 933121A/G
1-4: 1121401G/C
1-4: 1135104A/G
1-4: 1149636T/A
1-4: 1250488T/C
1-4: 1260751G/C
1-4: 1408066C/T
1-4: 1409311A/G
1-4: 1409523C/A
1-4: 1644135G/C
1-4: 1644159A/T
1-4: 1673170A/G
1-4: 1706386A/T
1-4: 1714518G/A
1-4: 1733171G/T
1-4: 1789296C/T
1-4: 1882140T/G
1-4: 1904444G/A
1-4: 1926945T/C
1-4: 1938633G/A
1-4: 1949919A/G
1-4: 2147599T/C
1-4: 2313063A/G
1-4: 2319820A/C
1-4: 2335218T/A
1-4: 2346800C/A
1-4: 2347142G/T
1-4: 2347511G/A
1-4: 2347526G/T
1-4: 2348016A/G
1-4: 2348533A/G
1-4: 2348878A/T
1-4: 2349046T/A
1-4: 2349277C/T
1-4: 2349378G/C
1-4: 2349494A/G
1-4: 2349562A/G
1-4: 2349595T/A
1-4: 2349724G/A
1-4: 2349824C/T
1-4: 2349919C/T
1-4: 2350040G/C
1-4: 2350066T/C
1-4: 2350162A/T
1-4: 2350270A/G
1-4: 2350381C/T
1-4: 2350493A/T
1-4: 2350804G/C
1-4: 2350937A/T
1-4: 2351046T/C
1-4: 2351139A/C
1-4: 2351147T/A
1-4: 2351651T/A
1-4: 2351669T/C
1-4: 2351772C/A
1-4: 2351927A/C
1-4: 2352184A/G
1-4: 2399556T/A
1-4: 2443365T/C
1-4: 2588576T/A
1-4: 2661243A/G
1-4: 2879857A/G
1-4: 2895383T/A
1-4: 2900822T/C
1-4: 2930933T/A
1-4: 2935139C/T
1-4: 2935150C/T
1-4: 2941694G/A
1-4: 3111514C/T
1-4: 3498306C/A
1-4: 3513271T/A
1-4: 3594900A/G
1-4: 3648416C/A
1-4: 3648428G/T
1-4: 3677277A/G
1-4: 3802065G/T
1-4: 4019375C/A
1-4: 4098489A/T
1-4: 4149376G/A
1-4: 4181405A/T
1-4: 4443268A/G
1-4: 4624265G/T
1-4: 4655362T/A
1-4: 4698013T/C
1-4: 4817828G/A
1-4: 4832201G/A
1-4: 4875761A/G
1-4: 4901837A/G
1-4: 5035411A/T
1-4: 5084119A/C
1-4: 5096153A/G
1-4: 5110460T/G
1-4: 5207096A/C
1-4: 5217116G/A
1-4: 5228814C/T
1-4: 5270831G/A
1-4: 5289001A/G
1-4: 5291241T/A
1-4: 5300175T/C
1-4: 5339337A/G
1-4: 5509350T/C
1-4: 5511670A/T
1-4: 5528781C/T
1-4: 5592589T/C
1-4: 5650033C/T
1-4: 5693130A/G
1-4: 5738829T/C
1-4: 5774991C/T
1-4: 6058728A/G
1-4: 6074760G/A
1-4: 6100583A/C
1-4: 6132630A/G
1-4: 6182807G/A
1-4: 6225360C/A
1-4: 6248130C/T
1-4: 6291222C/T
1-4: 6313425A/C
1-4: 6500972T/G
1-4: 6555104C/T
1-4: 6566838T/C
1-4: 6580164A/G
1-4: 6581290G/A
1-4: 6581291C/T
1-4: 6581294C/T
1-4: 6581312C/T
1-4: 6655915C/T
1-4: 6721127T/A
1-4: 6755015A/G
1-4: 6772063T/A
1-4: 6803921T/G
1-4: 6814465C/T
1-4: 6826565C/A
1-4: 6869110T/C
1-4: 6929404G/C
1-4: 7065290C/T
1-4: 7123959T/C
1-4: 7150904C/T
1-4: 7324464G/T
1-4: 7405523G/A
1-4: 7430694T/G
1-4: 7531417T/A
1-4: 7544268A/G
1-4: 7573485A/C
1-4: 7582331G/A
1-4: 7619360G/A
1-4: 7661679T/C
1-4: 7698775C/T
1-4: 7709439A/T
1-4: 7722101G/C
1-4: 7774306G/A
1-4: 7810572T/C
1-4: 7849436G/A
1-4: 7868472G/A
1-4: 7895761C/T
1-4: 7930828G/C
1-4: 7965237G/A
1-4: 8053481A/G
1-4: 8147278C/A
1-4: 8159506C/T
1-4: 8212832A/T
1-4: 8258259T/G
1-4: 8339046A/G
1-4: 8380245G/A
1-4: 8380778A/G
1-4: 8439780A/T
1-4: 8541270A/T
1-4: 8696880C/T
1-4: 8752282C/A
1-4: 8900845T/A
1-4: 8922143C/T
1-4: 8973310C/T
1-4: 9020769T/A
1-4: 9117818C/T
1-4: 9239197G/A
1-4: 9382427G/A
1-4: 9436886C/T
1-4: 9463437G/A
1-4: 9571942C/T
1-4: 9917255C/T
1-4: 10027086A/G
1-4: 10071155T/C
1-4: 10132668G/A
1-4: 10188814A/T
1-4: 10282088A/G
1-4: 10336805A/G
1-4: 10399818G/A
1-4: 10464704A/G
1-4: 10567040G/T
1-4: 10670031T/C
1-4: 10672004G/C
1-4: 10833185A/T
1-4: 10890046T/A
1-4: 10891376T/G
1-4: 10916775G/A
1-4: 11060523T/C
1-4: 11072735C/T
1-4: 11111700C/T
1-4: 11122009C/A
1-4: 11133624C/G
1-4: 11133650C/T
1-4: 11133775A/G
1-4: 11134936G/T
1-4: 11144863T/C
1-4: 11155051T/A
1-4: 11165709A/C
1-4: 11407309C/T
1-4: 11488053T/C
1-4: 11545689G/A
1-4: 11545832C/T
1-4: 11555034C/G
1-4: 11555165A/C
1-4: 11572886T/A
1-4: 11620396A/G
1-4: 11632362C/T
1-4: 11643422C/T
1-4: 11655987C/T
1-4: 11657020G/A
1-4: 11667313G/A
1-4: 11705452C/T
1-4: 11727841C/G
1-4: 11728262T/C
1-4: 11756394C/G
1-4: 11767468G/T
1-4: 11827106A/T
1-4: 11860346T/C
1-4: 11864031C/T
1-4: 11864172A/C
1-4: 11865251G/A
1-4: 11867894A/G
1-4: 11871971C/T
1-4: 11872188C/A
1-4: 11872489C/T
1-4: 11872492T/C
1-4: 11872754T/C
1-4: 11898730C/T
1-4: 11934236G/A
1-4: 11941508T/A
1-4: 11947199T/G
1-4: 11970709G/A
1-4: 11986687A/C
1-4: 12000218A/C
1-4: 12015325G/A
1-4: 12026358A/G
1-4: 12026469G/T
1-4: 12063998A/G
1-4: 12083119G/A
1-4: 12169576A/C
1-4: 12199170T/G
1-4: 12224544T/A
1-4: 12224997T/C
1-4: 12226500G/A
1-4: 12304049C/T
1-4: 12368088T/C
1-4: 12398920C/G
1-4: 12418419G/A
1-4: 12530157T/A
1-4: 12537375T/C
1-4: 12552436G/A
1-4: 12569943A/G
1-4: 12572730A/G
1-4: 12640627A/G
1-4: 12731227A/C
1-4: 12786196A/G
1-4: 12866194C/T
1-4: 12897343G/C
1-4: 12937616C/A
1-4: 13094897C/T
1-4: 13144195G/A
1-4: 13179709G/A
1-4: 13224188C/T
1-4: 13225447A/T
1-4: 13225574G/A
1-4: 13225589G/A
1-4: 13225803G/A
1-4: 13225953T/C
1-4: 13249789A/G
1-4: 13298671G/A
1-4: 13361309T/C
1-4: 13412634G/A
1-4: 13468756A/G
1-4: 13527663A/T
1-4: 13608261G/T
1-4: 13727598C/T
1-4: 13775698A/G
1-4: 13915121A/G
1-4: 13919726A/T
1-4: 13939286C/T
1-4: 13955698A/T
1-4: 13965940G/A
1-4: 13998718G/A
1-4: 14024144T/C
1-4: 14084499T/A
1-4: 14137888C/A
1-4: 14193902T/G
1-4: 14230533G/T
1-4: 14265270C/T
1-4: 14275676T/A
1-4: 14303446T/G
1-4: 14322102T/A
1-4: 14365144T/A
1-4: 14365177C/G
1-4: 14366500C/G
1-4: 14366506T/A
1-4: 14366564G/A
1-4: 14366733G/A
1-4: 14366879T/C
1-4: 14375215T/A
1-4: 14426652G/T
1-4: 14431211C/T
1-4: 14431227T/A
1-4: 14445250C/A
1-4: 14482629T/C
1-4: 14483270T/G
1-4: 14727325C/A
1-4: 14791711A/G
1-4: 14839564T/C
1-4: 14915717T/A
1-4: 15030237A/G
1-4: 15193480C/T
1-4: 15214065A/G
1-4: 15219914T/A
1-4: 15250354G/A
1-4: 15282719G/A
1-4: 15338395G/A
1-4: 15383917C/T
1-4: 15415147C/T
1-4: 15426649G/T
1-4: 15428312A/G
1-4: 15503294G/A
1-4: 15521438T/C
1-4: 15542939G/A
1-4: 15574144G/T
1-4: 15579200G/A
1-4: 15579236C/T
1-4: 15595769A/G
1-4: 15606592A/G
1-4: 15618062T/C
1-4: 15655929C/T
1-4: 15688568T/G
1-4: 15738038G/A
1-4: 15755771T/C
1-4: 15758062G/A
1-4: 15831803A/T
1-4: 15860322G/A
1-4: 15872711T/A
1-4: 15874022C/T
1-4: 15935883T/G
1-4: 15987882T/C
1-4: 16004595C/T
1-4: 16034021T/A
1-4: 16043159T/C
1-4: 16043228C/A
1-4: 16072270T/G
1-4: 16094698A/C
1-4: 16140018A/G
1-4: 16189342T/A
1-4: 16227598T/C
1-4: 16267349G/A
1-4: 16292898T/G
1-4: 16319328C/T
1-4: 16343432T/G
1-4: 16424809C/T
1-4: 16463649T/G
1-4: 16501521A/T
1-4: 16531223G/A
1-4: 16606256G/T
1-4: 16672396A/T
1-4: 16770744T/C
1-4: 16783584G/A
1-4: 16798562T/C
1-4: 16811799G/A
1-4: 16822773T/C
1-4: 16948743C/T
1-4: 17025442G/A
1-4: 17085574T/A
1-4: 17150284T/C
1-4: 17221503C/T
1-4: 17250565T/C
1-4: 17491868C/T
1-4: 17622206C/T
1-4: 17642380A/C
1-4: 17682804G/A
1-4: 17756084G/A
1-4: 17824665C/G
1-4: 17853309C/T
1-4: 17886485C/T
1-4: 17900515A/G
1-4: 17925335T/C
1-4: 17925553A/T
1-4: 17925555G/A
1-4: 17925657G/A
1-4: 17936147A/G
1-4: 17936619C/A
1-4: 17936680A/G
1-4: 17953182C/T
1-4: 17957013T/G
1-4: 17957252A/T
1-4: 17992751C/G
1-4: 17992809G/A
1-4: 18058173C/T
1-4: 18091507G/A
1-4: 18093150C/T
1-4: 18179400C/T
1-4: 18192544A/T
1-4: 18267690A/G
1-4: 18297445T/A
1-4: 18348273C/G
1-4: 18367793A/G
1-4: 18373539T/G
1-4: 18391247C/A
1-4: 18429876A/G
1-4: 18690754G/A
1-4: 18710314G/A
1-4: 18715041C/G
1-4: 18750977C/T
1-4: 18775968T/C
1-4: 18811320G/A
1-4: 18837971A/G
1-4: 18880368G/A
1-4: 18962682G/A
1-4: 18965469G/C
1-4: 18965573A/G
1-4: 18965720G/A
1-4: 18966848A/G
1-4: 18966964G/T
1-4: 18976384A/T
1-4: 18981930T/C
1-4: 19017780C/T
1-4: 19036711T/C
1-4: 19094970T/C
1-4: 19126348A/C
1-4: 19163954G/T
1-4: 19232712T/A
1-4: 19263415A/G
1-4: 19310847A/T
1-4: 19338433T/A
1-4: 19346531A/T
1-4: 19346563T/C
1-4: 19384306A/G
1-4: 19498574A/T
1-4: 19510757T/G
1-4: 19555361C/G
1-4: 19603741A/C
1-4: 19707439C/T
1-4: 19717656G/T
1-4: 19731356C/A
1-4: 19808072G/A
1-4: 19855142C/T
1-4: 19875455G/T
1-4: 19885759C/T
1-4: 19916229C/T
1-4: 19939239T/C
1-4: 19963016A/G
1-4: 19975315T/C
1-4: 20039825C/T
1-4: 20106236T/A
1-4: 20146928A/G
1-4: 20179707A/G
1-4: 20278655T/G
1-4: 20327711C/G
1-4: 20347627T/C
1-4: 20350070C/T
1-4: 20350695G/A
1-4: 20370243A/T
1-4: 20384423A/G
1-4: 20420931A/G
1-4: 20452712T/C
1-4: 20457515T/C
1-4: 20474924T/A
1-4: 20558524C/T
1-4: 20602889T/A
1-4: 20650389T/A
1-4: 20652343G/.
1-4: 20673193T/C
1-4: 20674158T/A
1-4: 20674424T/A
1-4: 20674529C/T
1-4: 20693392T/G
1-4: 20715097T/A
1-4: 20729757A/G
1-4: 20730127A/C
1-4: 20730131T/C
1-4: 20730380A/G
1-4: 20746600T/C
1-4: 20768412C/A
1-4: 20814618T/A
1-4: 20827505A/G
1-4: 20875741A/G
1-4: 20889429G/A
1-4: 20957268A/G
1-4: 20989904T/A
1-4: 21024043C/T
1-4: 21067978T/A
1-4: 21079736T/C
1-4: 21080296T/C
1-4: 21101016A/G
1-4: 21165547A/G
1-4: 21215096A/T
1-4: 21274293G/A
1-4: 21370027G/A
1-4: 21477860A/G
1-4: 21499082G/A
1-4: 21554378T/C
1-4: 21575118A/G
1-4: 21586980A/G
1-4: 21620366G/A
1-4: 21661787A/G
1-4: 21689240G/A
1-4: 21811477G/A
1-4: 21829709G/A
1-4: 21843621T/G
1-4: 21927950G/A
1-4: 21995834T/C
1-4: 22006968A/G
1-4: 22017241C/T
1-4: 22039066C/T
1-4: 22042369C/A
1-4: 22067258A/C
1-4: 22084989C/T
1-4: 22103250T/C
1-4: 22104464G/A
1-4: 22198811C/T
1-4: 22268603A/G
1-4: 22330593A/G
1-4: 22365031C/T
1-4: 22389444G/A
1-4: 22389686C/T
1-4: 22389882T/C
1-4: 22395947A/G
1-4: 22402358G/A
1-4: 22426605A/G
1-4: 22440649T/C
1-4: 22471752A/T
1-4: 22486074G/A
1-4: 22486373G/A
1-4: 22500785C/A
1-4: 22537154T/C
1-4: 22593096C/A
1-4: 22618828T/A
1-4: 22630174T/C
1-4: 22633596A/C
1-4: 22633654C/T
1-4: 22643216T/C
1-4: 22693696C/T
1-4: 22703227C/T
1-4: 22703593T/A
1-4: 22704891G/A
1-4: 22708411T/C
1-4: 22872611C/T
1-4: 23010477G/A
1-4: 23010630C/G
1-4: 23011646C/T
1-4: 23155776A/T
1-4: 23245316G/A
1-4: 23278269G/A
1-4: 23331756G/T
1-4: 23337721A/T
1-4: 23353415C/T
1-4: 23390084G/A
1-4: 23421107A/C
1-4: 23461503A/G
1-4: 23472257C/T
1-4: 23484483T/C
1-4: 23535104G/T
1-4: 23543557T/C
1-4: 23548573T/A
1-4: 23609651T/C
1-4: 23689219T/A
1-4: 23689309C/T
1-4: 23725260A/T
1-4: 23772080A/G
1-4: 23814924A/G
1-4: 23833331T/C
1-4: 23867145G/A
1-4: 23868349G/T
1-4: 23868454C/A
1-4: 23879032T/C
1-4: 23906613G/T
1-4: 23948576C/G
1-4: 24011937G/A
1-4: 24041042C/A
1-4: 24042389T/A
1-4: 24052903A/G
1-4: 24069260C/T
1-4: 24108552G/T
1-4: 24126657G/T
1-4: 24144564A/G
1-4: 24151239C/G
1-4: 24185099G/T
1-4: 24212467T/C
1-4: 24474608T/C
1-4: 24478935A/G
1-4: 24487954G/C
1-4: 24502887C/A
1-4: 24558234A/T
1-4: 24592166T/C
1-4: 24609659G/C
1-4: 24636947A/G
1-4: 24637050T/C
1-4: 24637100G/C
1-4: 24637143T/C
1-4: 24774081C/T
1-4: 24907907C/T
1-4: 24934569T/C
1-4: 24984035T/G
1-4: 25170737T/G
1-4: 25225572C/T
1-4: 25263371G/A
1-4: 25417239A/G
1-4: 25456077C/T
1-4: 25497487T/G
1-4: 25517195G/A
1-4: 25532700A/G
1-4: 25563844G/A
1-4: 25581917G/A
1-4: 25635343G/A
1-4: 25646913G/A
1-4: 25694720A/C
1-4: 25767629C/T
1-4: 25822595G/T
1-4: 25845486T/G
1-4: 25867092T/C
1-4: 25893407A/C
1-4: 25983737G/T
1-4: 26054887A/T
1-4: 26071816C/G
1-4: 26128371C/A
1-4: 26139770T/C
1-4: 26184183A/G
1-4: 26264461G/A
1-4: 26264845T/A
1-4: 26291662T/C
1-4: 26298613C/A
1-4: 26334246C/T
1-4: 26353036A/G
1-4: 26489342A/T
1-4: 26570021G/T
1-4: 26580265A/G
1-4: 26620513T/A
1-4: 26732499C/T
1-4: 26750321G/A
1-4: 26846315C/A
1-4: 26888914A/G
1-4: 26899668A/G
1-4: 26911170A/G
1-4: 26939587G/A
1-4: 27027618C/T
1-4: 27027780T/A
1-4: 27027906A/T
1-4: 27040679C/T
1-4: 27041468A/T
1-4: 27050822T/C
1-4: 27085996T/A
1-4: 27113786G/A
1-4: 27166501T/A
1-4: 27272014C/G
1-4: 27293752T/C
1-4: 27317588C/T
1-4: 27339960C/A
1-4: 27378623C/T
1-4: 27395782C/T
1-4: 27429646G/A
1-4: 27473476G/A
1-4: 27507851A/C
1-4: 27556280T/C
1-4: 27559661G/A
1-4: 27595422A/G
1-4: 27626353C/A
1-4: 27661036A/C
1-4: 27686609G/A
1-4: 27737557T/A
1-4: 27770107A/G
1-4: 27930255A/G
1-4: 27949059G/A
1-4: 27980265C/A
1-4: 28012291G/A
1-4: 28012493G/T
1-4: 28012704T/A
1-4: 28032263A/T
1-4: 28089964A/G
1-4: 28128611T/C
1-4: 28182103A/C
1-4: 28202380G/C
1-4: 28241721A/T
1-4: 28254224G/A
1-4: 28273437G/A
1-4: 28275127T/C
1-4: 28336396A/C
1-4: 28405110C/T
1-4: 28447828A/C
1-4: 28509521C/A
1-4: 28654038C/A
1-4: 28738432G/A
1-4: 28757810C/T
1-4: 28757994T/C
1-4: 28778271T/C
1-4: 28794286C/T
1-4: 28861487G/T
1-4: 28874123C/A
1-4: 28901283T/C
1-4: 28989427C/A
1-4: 29004240T/A
1-4: 29026352T/A
1-4: 29030294G/A
1-4: 29073051C/A
1-4: 29073297A/T
1-4: 29091358C/A
1-4: 29108054T/C
1-4: 29122939A/T
1-4: 29173627G/T
1-4: 29206463G/A
1-4: 29238397C/T
1-4: 29326043A/G
1-4: 29390639G/A
1-4: 29474604T/G
1-4: 29593027G/T
1-4: 29730789T/C
1-4: 29767668T/A
1-4: 29818692G/A
1-4: 29847755A/T
1-4: 29944674A/C
1-4: 30020098C/A
1-4: 30091001C/T
1-4: 30111232G/T
1-4: 30131377T/A
1-4: 30170144C/T
1-4: 30201329C/A
1-4: 30260234C/T
1-4: 30306412C/T
1-4: 30318545A/T
1-4: 30329438T/G
1-4: 30377953T/A
1-4: 30421426A/G
1-4: 30455288T/G
1-4: 30495861T/G
1-4: 30507450A/G
1-4: 30575594C/T
1-4: 30637843G/C
1-4: 30928884T/C
1-4: 31025367C/T
1-4: 31040429A/G
1-4: 31067544T/C
1-4: 31126576C/T
1-4: 31166320T/A
1-4: 31178823G/A
1-4: 31320727G/A
1-4: 31339913G/A
1-4: 31377945G/A
1-4: 31397030A/G
1-4: 31397168A/G
1-4: 31397275T/G
1-4: 31411113C/G
1-4: 31442173C/T
1-4: 31492894C/T
1-4: 31536775T/C
1-4: 31620404G/A
1-4: 31646413A/G
1-4: 31692917A/G
1-4: 31776120A/G
1-4: 31787169G/A
1-4: 31798077A/G
1-4: 31814477T/G
1-4: 31858236C/T
1-4: 31866677G/A
1-4: 31866688C/T
1-4: 31898254T/C
1-4: 31946889T/A
1-4: 31995827G/A
1-4: 32085407A/G
1-4: 32128573C/T
1-4: 32282509A/G
1-4: 32594163T/C
1-4: 32636821T/C
1-4: 32780255T/C
1-4: 32881318G/A
1-4: 32882660G/C
1-4: 32883111C/T
1-4: 32883229T/A
1-4: 32900714A/G
1-4: 32919535A/G
1-4: 33105688A/T
1-4: 33163114G/A
1-4: 33312909C/T
1-4: 33322948G/T
1-4: 33434790C/G
1-4: 33448876G/T
1-4: 33473684C/T
1-4: 33567124C/T
1-4: 33658249A/C
1-4: 33712746T/G
1-4: 33756930G/A
1-4: 33761458C/A
1-4: 33767145G/A
1-4: 33808072G/A
1-4: 33842013T/C
1-4: 33911045G/A
1-4: 33938808T/G
1-4: 34168050C/T
1-4: 34260657C/T
1-4: 34280011A/G
1-4: 34390641C/T
1-4: 34437969C/T
1-4: 34758308T/C
1-4: 34893198G/A
1-4: 34979459C/T
1-4: 35065957T/C
1-4: 35166308T/C
1-4: 35230985C/T
1-4: 35242175G/A
1-4: 35357157A/G
1-4: 35374978G/A
1-4: 35401483C/T
1-4: 35465024C/T
1-4: 35504336A/G
1-4: 35511912A/T
1-4: 35522175T/C
1-4: 35680770T/G
1-4: 35735954G/A
1-4: 35794061C/T
1-4: 35932587T/C
1-4: 35953521G/A
1-4: 36051925A/G
1-4: 36081191T/A
1-4: 36111843C/T
1-4: 36146861T/A
1-4: 36165523A/T
1-4: 36165725A/G
1-4: 36166434C/T
1-4: 36200014T/C
1-4: 36220040G/A
1-4: 36233343C/T
1-4: 36248009T/G
1-4: 36276569G/A
1-4: 36434558A/G
1-4: 36536051C/A
1-4: 36550874T/A
1-4: 36559447T/A
1-4: 36575966G/A
1-4: 36597665C/T
1-4: 36684011G/T
1-4: 36735613T/C
1-4: 36746441C/T
1-4: 36808568C/T
1-4: 36834886T/C
1-4: 36866129A/T
1-4: 36932120G/A
1-4: 36945385C/T
1-4: 36962319A/G
1-4: 36964608A/G
1-4: 36964733T/A
1-4: 36964867A/G
1-4: 37042679G/A
1-4: 37076324A/G
1-4: 37115037A/G
1-4: 37129577C/T
1-4: 37140465A/G
1-4: 37166612T/C
1-4: 37238464C/A
1-4: 37260948T/C
1-4: 37311312T/C
1-4: 37355053G/A
1-4: 37396210C/T
1-4: 37423915C/T
1-4: 37425564G/A
1-4: 37425871G/A
1-4: 37512696G/A
1-4: 37513022T/C
1-4: 37513030A/C
1-4: 37516549T/A
1-4: 37541175A/G
1-4: 37606851T/C
1-4: 37668194C/T
1-4: 37725774C/T
1-4: 37787696C/T
1-4: 37885079C/T
1-4: 37910385C/A
1-4: 37927218A/G
1-4: 37977385T/C
1-4: 38039607T/A
1-4: 38058332T/A
1-4: 38070582A/C
1-4: 38153282G/A
1-4: 38212208T/C
1-4: 38224928G/T
1-4: 38496991C/T
1-4: 38510861T/G
1-4: 38527122A/G
1-4: 38542618T/A
1-4: 38550664A/G
1-4: 38553190G/T
1-4: 38553322C/T
1-4: 38553469C/T
1-4: 38553557G/C
1-4: 38555745C/A
1-4: 38557123G/A
1-4: 38557699G/A
1-4: 38620438G/A
1-4: 38620560A/T
1-4: 38639535G/A
1-4: 38662493A/T
1-4: 38684542A/G
1-4: 38816412T/A
1-4: 38817789C/T
1-4: 38828454G/A
1-4: 38853852C/T
1-4: 38921463C/T
1-4: 38961251T/C
1-4: 38994633A/T
1-4: 39007649T/C
1-4: 39052149G/A
1-4: 39053130C/T
1-4: 39082633A/T
1-4: 39084795A/G
1-4: 39097723T/G
1-4: 39115693C/T
1-4: 39197226G/T
1-4: 39221815T/G
1-4: 39222348A/T
1-4: 39222369T/C
1-4: 39222557G/A
1-4: 39223223T/C
1-4: 39245700A/G
1-4: 39255959C/G
1-4: 39295153A/C
1-4: 39317678T/C
1-4: 39357935A/G
1-4: 39387474A/G
1-4: 39410530A/G
1-4: 39440835G/T
1-4: 39474023C/T
1-4: 39484661G/A
1-4: 39625477A/C
1-4: 39627293A/T
1-4: 39642771A/C
1-4: 39710731T/C
1-4: 39770537A/G
1-4: 39795972C/T
1-4: 39833771G/A
1-4: 39879988C/T
1-4: 39949196A/T
1-4: 39957177C/G
1-4: 39957289T/A
1-4: 39957441A/G
1-4: 39962005A/G
1-4: 39962404A/T
1-4: 39977713G/A
1-4: 39978032C/T
1-4: 40108608A/G
1-4: 40110046A/C
1-4: 40237170C/A
1-4: 40285793G/A
1-4: 40287394C/T
1-4: 40330977T/C
1-4: 40421651G/A
1-4: 40423067G/C
1-4: 40423202G/C
1-4: 40426616C/T
1-4: 40433182G/A
1-4: 40434860A/G
1-4: 40501524A/G
1-4: 40541875C/T
1-4: 40574050A/G
1-4: 40588181T/C
1-4: 40589467G/A
1-4: 40589876C/A
1-4: 40715415G/A
1-4: 40728782A/T
1-4: 40807848C/T
1-4: 40808530T/G
1-4: 40821700G/A
1-4: 40849915A/G
1-4: 40879748C/G
1-4: 40919084C/T
1-4: 40932936T/A
1-4: 40986187A/G
1-4: 41034584A/G
1-4: 41051433C/T
1-4: 41062182G/T
1-4: 41085894A/T
1-4: 41101355A/G
1-4: 41101614T/C
1-4: 41150067T/G
1-4: 41287990G/T
1-4: 41319973T/C
1-4: 41399411T/C
1-4: 41434541A/G
1-4: 41487384C/T
1-4: 41489252T/A
1-4: 41516975T/A
1-4: 41583014T/C
1-4: 41632611A/G
1-4: 41680326G/A
1-4: 41722021T/C
1-4: 41772543C/G
1-4: 41799790A/C
1-4: 41928574T/C
1-4: 41982585A/G
1-4: 42026722A/G
1-4: 42108189A/G
1-4: 42108381G/A
1-4: 42155942A/G
1-4: 42182267A/T
1-4: 42273452C/T
1-4: 42294008G/A
1-4: 42367809G/A
1-4: 42453423T/C
1-4: 42490036G/A
1-4: 42581433T/C
1-4: 42663986A/G
1-4: 42858146T/C
1-4: 42894882A/G
1-4: 42922150C/T
1-4: 43048834A/T
1-4: 43159856C/T
1-4: 43167546A/G
1-4: 43243921T/C
1-4: 43273675G/A
1-4: 43350609T/A
1-4: 43363817T/C
1-4: 43380541T/C
1-4: 43386073G/T
1-4: 43386122C/A
1-4: 43404814A/C
1-4: 43407104G/T
1-4: 43439872A/C
1-4: 43453934T/C
1-4: 43489847G/T
1-4: 43535768A/G
1-4: 43633893T/C
1-4: 43667906T/G
1-4: 43679552C/A
1-4: 43704426T/C
1-4: 43789138G/A
1-4: 43942889G/A
1-4: 44038422C/T
1-4: 44077386T/A
1-4: 44196657G/A
1-4: 44334321A/G
1-4: 44458561G/A
1-4: 44463104A/T
1-4: 44645170G/C
1-4: 44648989G/A
1-4: 44678610C/T
1-4: 44747475C/T
1-4: 44774989A/T
1-4: 44792090C/T
1-4: 44831812A/G
1-4: 44877646C/T
1-4: 44910220A/G
1-4: 44940162G/A
1-4: 44964807G/A
1-4: 45387579C/T
1-4: 45540268T/G
1-4: 45693750G/A
1-4: 45980266T/C
1-4: 46025789C/G
1-4: 46076401T/C
1-4: 46201906A/G
1-4: 46285773A/G
1-4: 46327699C/A
1-4: 46395468A/T
1-4: 46471471T/C
1-4: 46499430T/C
1-4: 46533736T/A
1-4: 46606712T/C
1-4: 46664565A/G
1-4: 46792709T/C
1-4: 46850678G/A
1-4: 46919797A/T
1-4: 47021469T/C
1-4: 47031858G/A
1-4: 47083227T/A
1-4: 47118835T/C
1-4: 47162038A/C
1-4: 47186977T/A
1-4: 47339050A/G
1-4: 47352361C/A
1-4: 47381664G/A
1-4: 47408900T/C
1-4: 47485300G/A
1-4: 47555611C/T
1-4: 47555859T/C
1-4: 47556474G/T
1-4: 47556682T/A
1-4: 47575720G/A
1-4: 47626927T/G
1-4: 47644188A/T
1-4: 47718634C/T
1-4: 47766735G/A
1-4: 47809228A/T
1-4: 47842196A/G
1-4: 47884820C/T
1-4: 47897745C/T
1-4: 47985706C/T
1-4: 48004501A/T
1-4: 48039825T/C
1-4: 48060051G/A
1-4: 48081424G/A
1-4: 48108422A/G
1-4: 48148438T/C
1-4: 48174783G/A
1-4: 48190317T/A
1-4: 48205530G/T
1-4: 48249474A/G
1-4: 48295798G/A
1-4: 48328012G/T
1-4: 48353282G/A
1-4: 48408196G/A
1-4: 48465292T/C
1-4: 48507977C/T
1-4: 48568386C/A
1-4: 48675428A/T
1-4: 48739081G/A
1-4: 48839896C/T
1-4: 48850417T/C
1-4: 48883943C/T
1-4: 48958993A/G
1-4: 49006760C/T
1-4: 49085120C/A
1-4: 49162150C/T
1-4: 49219002T/A
1-4: 49371409C/T
1-4: 49393169A/G
1-4: 49480593C/G
1-4: 49630858C/T
1-4: 49727310C/T
1-4: 49842510T/C
1-4: 49881217C/T
1-4: 49918760T/A
1-4: 50030848G/A
1-4: 50103789A/T
1-4: 50119518C/T
1-4: 50156002G/T
1-4: 50227019G/T
1-4: 50280179T/C
1-4: 50342476G/A
1-4: 50410640G/A
1-4: 50440334T/C
1-4: 50496272T/C
1-4: 50551929G/A
1-4: 50577509C/T
1-4: 50649643G/A
1-4: 50716262G/A
1-4: 50764343A/T
1-4: 50774282A/T
1-4: 50822283G/A
1-4: 50826598T/C
1-4: 50872619T/A
1-4: 50884363G/A
1-4: 50907861C/T
1-4: 50928651T/C
1-4: 50942057G/C
1-4: 50962353G/A
1-4: 50980814A/G
1-4: 51011368G/T
1-4: 51065111A/G
1-4: 51077560T/C
1-4: 51165437G/A
1-4: 51179742G/T
1-4: 51206810T/C
1-4: 51338593C/T
1-4: 51341719G/A
1-4: 51363679G/A
1-4: 51397631C/T
1-4: 51420668T/C
1-4: 51449076C/T
1-4: 51487383T/C
1-4: 51565261T/C
1-4: 51614137A/T
1-4: 51647521A/G
1-4: 51647673A/G
1-4: 51667761G/A
1-4: 51704984T/G
1-4: 51795696G/A
1-4: 51806236G/A
1-4: 51905736T/C
1-4: 51958937C/T
1-4: 52036693T/A
1-4: 52081702G/A
1-4: 52145165G/A
1-4: 52151373T/C
1-4: 52224498G/A
1-4: 52393981A/C
1-4: 52424158A/C
1-4: 52554719A/T
1-4: 52576260C/T
1-4: 52576389A/T
1-4: 52577342C/T
1-4: 52606879T/C
1-4: 52654120T/C
1-4: 52704310A/G
1-4: 52736540T/C
1-4: 52783607T/C
1-4: 52818319A/C
1-4: 52850301A/G
1-4: 52892306A/G
1-4: 52911256T/C
1-4: 52964801T/G
1-4: 52976080T/C
1-4: 52991307G/T
1-4: 53014559A/G
1-4: 53017654T/C
1-4: 53018345G/A
1-4: 53018845G/C
1-4: 53019295G/A
1-4: 53019402C/T
1-4: 53019643G/A
1-4: 53036289T/C
1-4: 53036975A/G
1-4: 53037191A/G
1-4: 53037336T/C
1-4: 53037762A/G
1-4: 53037900T/C
1-4: 53038105C/T
1-4: 53049016G/A
1-4: 53064891T/G
1-4: 53093374G/A
1-4: 53153209C/T
1-4: 53204630A/T
1-4: 53260021G/A
1-4: 53315878G/A
1-4: 53403563G/A
1-4: 53482541A/T
1-4: 53530542T/C
1-4: 53578669T/A
1-4: 53594905A/G
1-4: 53605180C/A
1-4: 53660793T/C
1-4: 53687595A/G
1-4: 53865794A/G
1-4: 54019715C/T
1-4: 54035924G/C
1-4: 54043551G/A
1-4: 54124232T/C
1-4: 54159874A/G
1-4: 54173961C/T
1-4: 54282077G/C
1-4: 54325094T/A
1-4: 54341190T/C
1-4: 54426195T/A
1-4: 54508243T/C
1-4: 54521767A/G
1-4: 54613930A/T
1-4: 54723284A/G
1-4: 54886914A/G
1-4: 54913702C/T
1-4: 54918172T/C
1-4: 54924093G/C
1-4: 54963279A/G
1-4: 54999084C/T
1-4: 55068600C/T
1-4: 55100095G/T
1-4: 55138139C/T
1-4: 55139439T/C
1-4: 55148353T/A
1-4: 55158941A/T
1-4: 55206943T/C
1-4: 55281579A/T
1-4: 55349402A/G
1-4: 55365063G/A
1-4: 55388092A/T
1-4: 55403621A/C
1-4: 55445169G/A
1-4: 55471717G/T
1-4: 55531576A/C
1-4: 55666608C/A
1-4: 55686416T/A
1-4: 55769743A/G
1-4: 55866967G/A
1-4: 55908398C/A
1-4: 55955913C/T
1-4: 55956015T/C
1-4: 55956553C/T
1-4: 55956637T/A
1-4: 55956950A/T
1-4: 55956991G/T
1-4: 55957005C/A
1-4: 55957227T/C
1-4: 55957701G/A
1-4: 55958264C/T
1-4: 55958375G/A
1-4: 55964121A/G
1-4: 56029460A/T
1-4: 56050121A/T
1-4: 56095584G/C
1-4: 56192823T/C
1-4: 56315968G/A
1-4: 56363129G/A
1-4: 56382293T/C
1-4: 56414428A/G
1-4: 56427521C/T
1-4: 56427760A/G
1-4: 56427854A/T
1-4: 56427876G/T
1-4: 56427885A/C
1-4: 56528733G/A
1-4: 56545999G/T
1-4: 56567255G/A
1-4: 56672024C/T
1-4: 56777662T/C
1-4: 56792942A/T
1-4: 56916286G/T
1-4: 56943047A/T
1-4: 57019929G/A
1-4: 57087595C/T
1-4: 57125939A/T
1-4: 57205211C/T
1-4: 57238739A/G
1-4: 57265316T/A
1-4: 57323829G/A
1-4: 57349215T/C
1-4: 57460414C/A
1-4: 57474234T/A
1-4: 57504925T/C
1-4: 57544984T/C
1-4: 57680486T/A
1-4: 57720019C/A
1-4: 57783346A/G
1-4: 57830870C/T
1-4: 57869689A/G
1-4: 57905413T/C
1-4: 57984520G/A
1-4: 58039514G/C
1-4: 58061768T/A
1-4: 58076473G/T
1-4: 58171758T/C
1-4: 58193556C/T
1-4: 58216553T/C
1-4: 58233820A/G
1-4: 58247632G/T
1-4: 58277939T/G
1-4: 58339984G/A
1-4: 58417960A/G
1-4: 58469398G/A
1-4: 58501446C/T
1-4: 58511520C/A
1-4: 58559593T/C
1-4: 58606855C/T
1-4: 58643593G/T
1-4: 58664091C/G
1-4: 58785067C/A
1-4: 58788450A/T
1-4: 58894312A/G
1-4: 58990245C/T
1-4: 59043624T/A
1-4: 59348299A/G
1-4: 59485843G/A
1-4: 59519668A/G
1-4: 59552546G/T
1-4: 59703922A/C
1-4: 59790468G/A
1-4: 59875788G/C
1-4: 59953731C/T
1-4: 59959847A/G
1-4: 60114048G/A
1-4: 60183928G/T
1-4: 60184064G/C
1-4: 60312884G/A
1-4: 60337605T/A
1-4: 60418321G/A
1-4: 60456225T/C
1-4: 60475137G/A
1-4: 60485340T/C
1-4: 60513201T/G
1-4: 60548119T/C
1-4: 60573225C/T
1-4: 60631294C/G
1-4: 60710379A/G
1-4: 60766117C/T
1-4: 60803883C/A
1-4: 60882228C/T
1-4: 60917711C/T
1-4: 61011062G/A
1-4: 61068997C/A
1-4: 61191006A/T
1-4: 61266264C/T
1-4: 61307739G/A
1-4: 61369186G/A
1-4: 61425279T/A
1-4: 61433309T/C
1-4: 61433733G/A
1-4: 61444537G/A
1-4: 61479162G/A
1-4: 61517548G/A
1-4: 61528433G/T
1-4: 61669655G/A
1-4: 61681377C/T
1-4: 61739074T/C
1-4: 61804270A/T
1-4: 61854040G/T
1-4: 61955017A/G
1-4: 61989159C/T
1-4: 62075078G/A
1-4: 62087561A/G
1-4: 62089334A/G
1-4: 62098424C/T
1-4: 62123596C/A
1-4: 62199828A/G
1-4: 62279359A/G
1-4: 62319227C/T
1-4: 62346012A/G
1-4: 62398103G/A
1-4: 62443034G/A
1-4: 62487256T/A
1-4: 62547953A/C
1-4: 62592119G/A
1-4: 62636073A/T
1-4: 62703915A/G
1-4: 62781442T/A
1-4: 62816804A/G
1-4: 62901917A/G
1-4: 62934488A/C
1-4: 63009540A/C
1-4: 63168539G/T
1-4: 63179433T/G
1-4: 63250755G/T
1-4: 63296042T/G
1-4: 63324139G/C
1-4: 63363157C/A
1-4: 63419322C/T
1-4: 63445649G/A
1-4: 63529990T/A
1-4: 63568381G/C
1-4: 63610777G/A
1-4: 63640468G/A
1-4: 63814828A/T
1-4: 63829422G/A
1-4: 63910328A/T
1-4: 64104405C/T
1-4: 64125676T/C
1-4: 64158947C/A
1-4: 64187696A/G
1-4: 64228245A/T
1-4: 64435784T/G
1-4: 64473112T/C
1-4: 64518018C/G
1-4: 64546168A/G
1-4: 64582528C/T
1-4: 64659013T/C
1-4: 64663067C/T
1-4: 64731081C/T
1-4: 64854371T/C
1-4: 64975209A/T
1-4: 64985768A/C
1-4: 65059360A/T
1-4: 65151558G/A
1-4: 65325255C/A
1-4: 65488087A/T
1-4: 65549338T/C
1-4: 65580306G/T
1-4: 65620942G/A
1-4: 65633034G/T
1-4: 65659228G/C
1-4: 65689463G/T
1-4: 65707431A/G
1-4: 65937182T/C
1-4: 66069533A/G
1-4: 66126245T/A
1-4: 66157711G/A
1-4: 66158751A/G
1-4: 66158761C/T
1-4: 66186256T/G
1-4: 66224490T/G
1-4: 66252933C/T
1-4: 66297686A/G
1-4: 66348584C/T
1-4: 66359427A/G
1-4: 66438820T/A
1-4: 66496850G/A
1-4: 66535872T/A
1-4: 66591194G/A
1-4: 66609736A/G
1-4: 66731545A/G
1-4: 66840299C/A
1-4: 66883884G/A
1-4: 66941701A/C
1-4: 66967537A/C
1-4: 67054354C/G
1-4: 67193438G/A
1-4: 67236468G/A
1-4: 67241688G/A
1-4: 67327291C/T
1-4: 67349184T/A
1-4: 67359274T/G
1-4: 67377394T/A
1-4: 67390370A/G
1-4: 67415138T/G
1-4: 67452523C/T
1-4: 67479800A/G
1-4: 67481183G/A
1-4: 67499252C/A
1-4: 67531930C/T
1-4: 67558681G/T
1-4: 67589048C/T
1-4: 67604119T/C
1-4: 67604590T/C
1-4: 67604598T/C
1-4: 67604623G/A
1-4: 67604672G/A
1-4: 67604695T/A
1-4: 67604714A/C
1-4: 67604729A/G
1-4: 67604731G/C
1-4: 67797111T/C
1-4: 67799149C/T
1-4: 67799549A/T
1-4: 67815830T/C
1-4: 67816304C/T
1-4: 67820692G/A
1-4: 67820743A/G
1-4: 67863865C/T
1-4: 67878050C/A
1-4: 67890324G/A
1-4: 67993370C/T
1-4: 67997440C/T
1-4: 68076130G/A
1-4: 68175430C/T
1-4: 68185540T/A
1-4: 68213004T/A
1-4: 68270504G/A
1-4: 68318677G/A
1-4: 68365378A/G
1-4: 68402773G/A
1-4: 68482095T/C
1-4: 68648970A/G
1-4: 68747581A/G
1-4: 68856737G/A
1-4: 68891151C/T
1-4: 68920702G/A
1-4: 68921265G/T
1-4: 69012992A/G
1-4: 69022406T/C
1-4: 69032420A/G
1-4: 69060783A/G
1-4: 69096848A/T
1-4: 69165994T/C
1-4: 69293403A/G
1-4: 69377613G/A
1-4: 69427708G/C
1-4: 69552621C/G
1-4: 69637349T/C
1-4: 69757831C/T
1-4: 69888203T/C
1-4: 69906267C/T
1-4: 70033824G/A
1-4: 70048088A/T
1-4: 70141935T/A
1-4: 70207802A/G
1-4: 70243507G/A
1-4: 70294666G/A
1-4: 70311842T/A
1-4: 70336886T/A
1-4: 70415829G/T
1-4: 70429606C/T
1-4: 70495206G/T
1-4: 70550119G/T
1-4: 70717999G/T
1-4: 70949074T/A
1-4: 70961447G/A
1-4: 70971786C/G
1-4: 70986494A/T
1-4: 70987877T/A
1-4: 70997037T/A
1-4: 70997147C/T
1-4: 71000802A/T
1-4: 71020227T/G
1-4: 71020863A/T
1-4: 71021019A/C
1-4: 71021025C/T
1-4: 71057909T/C
1-4: 71097397T/G
1-4: 71160004A/G
1-4: 71361115G/A
1-4: 71379855A/G
1-4: 71621008A/G
1-4: 71800962A/T
1-4: 71885184A/G
1-4: 71921326G/A
1-4: 72018905G/A
1-4: 72123214A/G
1-4: 72225811A/G
1-4: 72257063G/A
1-4: 72291778A/C
1-4: 72305873T/C
1-4: 72396665G/A
1-4: 72410633G/A
1-4: 72548385A/G
1-4: 72549977T/C
1-4: 72678203G/C
1-4: 72682341A/G
1-4: 72911772A/T
1-4: 73054352A/G
1-4: 73146447T/G
1-4: 73366818A/C
1-4: 73394528G/A
1-4: 73526662T/C
1-4: 73737712C/A
1-4: 73902010G/C
1-4: 73916956C/T
1-4: 73940161C/G
1-4: 73946866A/G
1-4: 73959962A/T
1-4: 74130279A/G
1-4: 74132656T/C
1-4: 74159006G/T
1-4: 74159858C/T
1-4: 74162003G/T
1-4: 74186451G/A
1-4: 74215594A/G
1-4: 74260660T/A
1-4: 74366914A/C
1-4: 74552740G/T
1-4: 74640954A/G
1-4: 74782337G/A
1-4: 74819151A/T
1-4: 74830660A/T
1-4: 74930618T/C
1-4: 75197491C/T
1-4: 75252969G/A
1-4: 75257729A/G
1-4: 75289541C/G
1-4: 75397386A/C
1-4: 75402352G/A
1-4: 75636770G/A
1-4: 75659434A/G
1-4: 75689469C/T
1-4: 75741030G/A
1-4: 75846053G/T
1-4: 75947956C/T
1-4: 76052236C/T
1-4: 76122511T/C
1-4: 76290247T/C
1-4: 76390622A/G
1-4: 76426280G/A
2-1: 172710A/G
2-1: 175481T/C
2-1: 175529G/A
2-1: 175531G/A
2-1: 556488A/T
2-1: 598866A/G
2-1: 678133G/A
2-1: 1072954A/G
2-1: 1105890T/A
2-1: 1318704A/G
2-1: 1521545T/C
2-1: 1537959G/A
2-1: 1657899G/C
2-1: 1677370A/G
2-1: 1857836G/A
2-1: 1921161C/A
2-1: 2223054T/C
2-1: 2388658T/C
2-1: 2441468G/A
2-1: 2485859C/G
2-1: 2540297C/T
2-1: 2656949T/A
2-1: 2951940A/C
2-1: 3007218T/C
2-1: 3017894A/C
2-1: 3061317G/T
2-1: 3213788T/C
2-1: 3215076T/C
2-1: 3348187T/A
2-1: 3382395T/C
2-1: 3448364A/T
2-1: 3519555A/G
2-1: 3625940C/G
2-1: 3668065C/A
2-1: 3795513C/T
2-1: 3809087T/G
2-1: 3874226A/G
2-1: 4053111T/C
2-1: 4209676C/T
2-1: 4227126C/T
2-1: 4414853C/T
2-1: 4521678C/G
2-1: 4588343A/C
2-1: 4705941A/C
2-1: 4755665A/G
2-1: 4767222A/G
2-1: 4809830T/C
2-1: 4900082A/G
2-1: 4957187T/A
2-1: 4973486A/G
2-1: 4984989C/G
2-1: 5084205G/A
2-1: 5284705T/A
2-1: 5302646A/C
2-1: 5352162T/C
2-1: 5401084A/G
2-1: 5432082A/T
2-1: 5457945C/T
2-1: 5506081A/G
2-1: 5547001G/A
2-1: 5598591C/T
2-1: 5675908A/C
2-1: 5708678G/A
2-1: 5751615C/G
2-1: 5771692A/G
2-1: 5811532A/G
2-1: 5936442G/A
2-1: 5946447C/A
2-1: 5962163C/A
2-1: 6126234A/G
2-1: 6163448T/A
2-1: 6191084T/C
2-1: 6218460A/G
2-1: 6283360T/C
2-1: 6333734T/C
2-1: 6380789A/G
2-1: 6472795A/G
2-1: 6475246C/T
2-1: 6526025A/T
2-1: 6592158G/A
2-1: 6631135T/A
2-1: 6720817T/C
2-1: 6812270G/A
2-1: 6899679A/T
2-1: 6945819G/A
2-1: 7059802T/C
2-1: 7107974T/C
2-1: 7132222T/C
2-1: 7136069A/T
2-1: 7200927C/A
2-1: 7244586G/A
2-1: 7414821T/C
2-1: 7426784G/A
2-1: 7435399G/C
2-1: 7447936T/A
2-1: 7462919A/T
2-1: 7476499T/C
2-1: 7505749A/C
2-1: 7647494G/A
2-1: 7772229T/A
2-1: 7774754T/A
2-1: 7851405A/T
2-1: 7992496A/G
2-1: 8046068G/C
2-1: 8109012C/T
2-1: 8192125C/T
2-1: 8293719G/A
2-1: 8359745G/A
2-1: 8385502T/C
2-1: 8424612A/G
2-1: 8578441A/G
2-1: 8603592C/A
2-1: 8614228T/C
2-1: 8649775G/A
2-1: 8674805G/C
2-1: 8725572T/C
2-1: 8765829C/T
2-1: 8765839G/A
2-1: 8830966C/A
2-1: 8871570A/G
2-1: 8945392G/A
2-1: 9021261C/T
2-1: 9050322A/G
2-1: 9163539T/C
2-1: 9172674T/G
2-1: 9173698A/T
2-1: 9173988C/T
2-1: 9174947G/A
2-1: 9185798A/C
2-1: 9205549C/A
2-1: 9215406T/A
2-1: 9239564C/T
2-1: 9281809T/C
2-1: 9444219C/T
2-1: 9474949G/A
2-1: 9504938T/C
2-1: 9507777C/T
2-1: 9507833C/T
2-1: 9507849C/T
2-1: 9508105G/A
2-1: 9508392T/G
2-1: 9508509C/T
2-1: 9508721T/C
2-1: 9508865T/C
2-1: 9508871C/T
2-1: 9509050C/T
2-1: 9509397T/C
2-1: 9509729A/T
2-1: 9509836G/T
2-1: 9509948G/A
2-1: 9510250A/T
2-1: 9510265T/C
2-1: 9510366C/T
2-1: 9510501C/A
2-1: 9510816C/A
2-1: 9510947C/T
2-1: 9511068T/G
2-1: 9511075C/T
2-1: 9511220T/G
2-1: 9511329C/A
2-1: 9511343G/A
2-1: 9511444C/T
2-1: 9511577A/G
2-1: 9511602C/G
2-1: 9513372A/T
2-1: 9513616G/A
2-1: 9515510A/T
2-1: 9577081T/C
2-1: 9666856G/A
2-1: 9698066T/G
2-1: 9839666G/A
2-1: 9913259C/T
2-1: 9945904A/C
2-1: 10041815C/T
2-1: 10096511G/T
2-1: 10216327A/T
2-1: 10273316C/G
2-1: 10307440G/A
2-1: 10347288A/G
2-1: 10395924A/T
2-1: 10430419A/G
2-1: 10467728C/T
2-1: 10535293C/T
2-1: 10582517A/G
2-1: 10594122T/C
2-1: 10647949A/C
2-1: 10799112C/G
2-1: 10810682G/A
2-1: 10846965T/C
2-1: 10939301T/G
2-1: 10986874C/T
2-1: 11044242T/G
2-1: 11107848T/C
2-1: 11132986A/G
2-1: 11192990C/T
2-1: 11252891G/A
2-1: 11280045C/T
2-1: 11329713G/A
2-1: 11371183G/A
2-1: 11565342G/A
2-1: 11611664C/T
2-1: 11865337G/T
2-1: 11895046G/A
2-1: 11944840T/G
2-1: 11975820G/C
2-1: 11987240T/C
2-1: 12083330T/A
2-1: 12143156T/A
2-1: 12280249A/G
2-1: 12300075G/A
2-1: 12476191G/A
2-1: 12559364T/C
2-1: 12734177A/T
2-1: 12804650C/T
2-1: 12825876G/A
2-1: 12865071A/T
2-1: 12895979A/G
2-1: 12953296G/A
2-1: 13021147A/G
2-1: 13102802G/A
2-1: 13136034A/C
2-1: 13149600G/A
2-1: 13156256T/C
2-1: 13207384G/A
2-1: 13235626T/C
2-1: 13289129G/T
2-1: 13307656T/C
2-1: 13339180T/A
2-1: 13342460C/A
2-1: 13403165A/G
2-1: 13429816A/C
2-1: 13482199C/T
2-1: 13521996T/G
2-1: 13687787C/A
2-1: 13879797C/T
2-1: 13947761C/T
2-1: 13958710G/A
2-1: 13994222G/A
2-1: 14008648C/T
2-1: 14011980A/G
2-1: 14012709A/T
2-1: 14024788T/C
2-1: 14089651A/G
2-1: 14143245C/T
2-1: 14162756A/G
2-1: 14247001C/T
2-1: 14344639G/A
2-1: 14344680C/G
2-1: 14344968G/A
2-1: 14380388C/T
2-1: 14411724A/G
2-1: 14454433G/T
2-1: 14581001C/A
2-1: 14617106A/G
2-1: 14686349T/C
2-1: 14755176C/T
2-1: 14863867G/A
2-1: 14891352A/C
2-1: 14967588T/A
2-1: 14974519T/C
2-1: 15019302T/A
2-1: 15067421G/A
2-1: 15113236T/G
2-1: 15133882T/C
2-1: 15149194T/C
2-1: 15264112T/G
2-1: 15266757A/T
2-1: 15275965T/A
2-1: 15276013T/C
2-1: 15278371T/G
2-1: 15459738A/G
2-1: 15485213C/T
2-1: 15530771T/A
2-1: 15732837C/A
2-1: 15747474T/A
2-1: 15786333C/T
2-1: 15812939G/T
2-1: 15899409C/T
2-1: 15912742C/G
2-1: 16039639C/T
2-1: 16091514C/T
2-1: 16103084A/G
2-1: 16133488G/T
2-1: 16169982G/T
2-1: 16170693C/T
2-1: 16191802G/A
2-1: 16251058T/C
2-1: 16353136C/T
2-1: 16413039G/A
2-1: 16432860C/T
2-1: 16461151A/G
2-1: 16511651G/A
2-1: 16633059T/C
2-1: 16651208T/C
2-1: 16689628T/G
2-1: 16717450A/G
2-1: 16781434G/A
2-1: 16869299G/A
2-1: 16914019G/A
2-1: 16930905T/A
2-1: 17001565C/G
2-1: 17023909A/G
2-1: 17127023T/G
2-1: 17140286T/C
2-1: 17161135A/G
2-1: 17188938A/G
2-1: 17247537A/C
2-1: 17248448G/A
2-1: 17248589C/G
2-1: 17249547G/A
2-1: 17255728A/G
2-1: 17265653T/C
2-1: 17308632G/T
2-1: 17317241G/A
2-1: 17317405A/T
2-1: 17319462G/C
2-1: 17329570A/G
2-1: 17380017A/G
2-1: 17447835C/T
2-1: 17486424G/A
2-1: 17631688T/G
2-1: 17631720A/C
2-1: 17631725A/G
2-1: 17712134G/A
2-1: 17726268C/T
2-1: 17854985A/G
2-1: 17871878G/A
2-1: 17892774A/T
2-1: 17992095C/T
2-1: 18005885T/C
2-1: 18027345A/G
2-1: 18045191T/A
2-1: 18055335G/A
2-1: 18086407A/G
2-1: 18173600T/C
2-1: 18238721C/T
2-1: 18421013G/T
2-1: 18430324C/T
2-1: 18444835C/T
2-1: 18467712A/G
2-1: 18490655G/T
2-1: 18543023A/G
2-1: 18616401A/G
2-1: 18633243A/C
2-1: 18667674T/C
2-1: 18762533A/G
2-1: 18774951A/T
2-1: 18904053C/A
2-1: 18919511C/T
2-1: 18932639A/G
2-1: 18957841A/G
2-1: 18971342A/G
2-1: 18999544C/G
2-1: 19094063T/A
2-1: 19117010C/A
2-1: 19117140A/G
2-1: 19157764T/C
2-1: 19170646A/T
2-1: 19183517A/T
2-1: 19199497T/G
2-1: 19276287A/C
2-1: 19310000C/T
2-1: 19344292C/A
2-1: 19352720G/A
2-1: 19371558A/G
2-1: 19382349C/A
2-1: 19384317C/T
2-1: 19394212T/C
2-1: 19433394T/G
2-1: 19462303A/G
2-1: 19522375A/G
2-1: 19563868C/T
2-1: 19629317G/A
2-1: 19666087G/C
2-1: 19741666A/C
2-1: 19848838G/A
2-1: 19854166T/C
2-1: 20001409T/A
2-1: 20017777C/T
2-1: 20064302A/C
2-1: 20065701C/T
2-1: 20069790T/C
2-1: 20069978C/T
2-1: 20070101G/A
2-1: 20070110A/G
2-1: 20092093A/T
2-1: 20138450C/T
2-1: 20172483C/T
2-1: 20267151A/C
2-1: 20303390C/A
2-1: 20465184A/G
2-1: 20565397T/A
2-1: 20676528A/G
2-1: 20701503C/A
2-1: 20719920G/A
2-1: 20731022A/T
2-1: 20758406G/A
2-1: 20870715G/T
2-1: 20940276C/A
2-1: 21082364C/T
2-1: 21082388A/T
2-1: 21103398T/G
2-1: 21164637G/A
2-1: 21164644C/T
2-1: 21165287T/C
2-1: 21165350T/C
2-1: 21165408C/T
2-1: 21165639C/A
2-1: 21166139A/T
2-1: 21166211T/A
2-1: 21166341G/T
2-1: 21166364G/A
2-1: 21166441C/T
2-1: 21166450A/G
2-1: 21166514A/G
2-1: 21166525C/A
2-1: 21192694T/G
2-1: 21258453C/T
2-1: 21477861T/C
2-1: 21644187A/G
2-1: 21693988T/C
2-1: 21761070T/G
2-1: 21786994C/T
2-1: 21852562G/A
2-1: 21958799T/C
2-1: 21961391A/G
2-1: 21977267T/G
2-1: 21997422C/T
2-1: 22008309G/A
2-1: 22089386A/G
2-1: 22117597C/T
2-1: 22150933A/G
2-1: 22206037C/T
2-1: 22206462G/A
2-1: 22206968T/G
2-1: 22207009T/A
2-1: 22207628G/A
2-1: 22239010A/G
2-1: 22254422G/C
2-1: 22266361A/T
2-1: 22282257A/T
2-1: 22329934A/T
2-1: 22332884A/T
2-1: 22333063A/T
2-1: 22333117A/T
2-1: 22420965G/A
2-1: 22526967A/G
2-1: 22593383A/T
2-1: 22629770T/C
2-1: 22649646G/T
2-1: 22692973C/T
2-1: 22714010T/A
2-1: 22749243C/G
2-1: 22796507C/T
2-1: 22798312C/A
2-1: 22814131T/C
2-1: 22839009G/A
2-1: 22850569C/G
2-1: 22889191G/A
2-1: 23038933A/T
2-1: 23040412T/C
2-1: 23049341G/A
2-1: 23052274A/T
2-1: 23063888T/C
2-1: 23073720C/T
2-1: 23085627G/A
2-1: 23118438T/G
2-1: 23132690G/A
2-1: 23149948C/T
2-1: 23214416A/G
2-1: 23272133A/G
2-1: 23293185G/A
2-1: 23310940C/T
2-1: 23350147T/C
2-1: 23377473G/A
2-1: 23410048T/C
2-1: 23466148G/A
2-1: 23468087C/G
2-1: 23496001G/A
2-1: 23551588A/C
2-1: 23551774T/C
2-1: 23639521T/A
2-1: 23693261G/A
2-1: 23762283C/G
2-1: 23806353C/A
2-1: 23874051G/T
2-1: 23913058T/G
2-1: 23913381A/G
2-1: 23913584C/T
2-1: 23913629A/C
2-1: 23915557A/G
2-1: 23915566T/C
2-1: 23915671G/A
2-1: 23926237G/A
2-1: 23969843C/A
2-1: 23969880C/A
2-1: 24003112G/A
2-1: 24025344T/G
2-1: 24056995T/C
2-1: 24086443A/G
2-1: 24087379T/C
2-1: 24091762G/A
2-1: 24096836G/A
2-1: 24100141A/G
2-1: 24109860G/A
2-1: 24143451G/A
2-1: 24171286C/T
2-1: 24272845T/A
2-1: 24290663G/T
2-1: 24341615C/G
2-1: 24343941T/G
2-1: 24405872T/G
2-1: 24462992G/A
2-1: 24463674G/A
2-1: 24463983C/T
2-1: 24465051C/A
2-1: 24465069T/G
2-1: 24515104G/T
2-1: 24543789C/G
2-1: 24634560G/C
2-1: 24657078T/C
2-1: 24695134G/T
2-1: 24746302T/C
2-1: 24758720G/T
2-1: 24809606A/T
2-1: 24839712G/T
2-1: 24843566A/T
2-1: 24865439A/T
2-1: 24906969A/C
2-1: 24919289T/G
2-1: 24965605T/C
2-1: 24997692A/G
2-1: 25010432T/A
2-1: 25042222G/C
2-1: 25044504G/A
2-1: 25044598T/C
2-1: 25069570A/T
2-1: 25073795G/A
2-1: 25076136G/A
2-1: 25076158T/C
2-1: 25079119A/G
2-1: 25084547A/G
2-1: 25113549A/G
2-1: 25175591T/C
2-1: 25193861G/C
2-1: 25268946G/A
2-1: 25287284G/T
2-1: 25288509C/A
2-1: 25297993T/G
2-1: 25316705C/T
2-1: 25342231A/G
2-1: 25349451G/T
2-1: 25349615C/A
2-1: 25350117T/A
2-1: 25353685T/A
2-1: 25384789G/A
2-1: 25665685G/T
2-1: 25768136G/A
2-1: 25805392G/T
2-1: 25833853A/G
2-1: 25848767C/T
2-1: 25852338G/T
2-1: 25866139C/T
2-1: 25886901T/C
2-1: 25899485C/T
2-1: 25971471C/T
2-1: 26003695G/A
2-1: 26036599T/A
2-1: 26048454A/C
2-1: 26098497G/A
2-1: 26184887T/C
2-1: 26236232G/C
2-1: 26283863A/C
2-1: 26385729G/C
2-1: 26396451G/A
2-1: 26414501A/C
2-1: 26504415T/C
2-1: 26546874C/T
2-1: 26562228A/T
2-1: 26581541T/.
2-1: 26599175T/A
2-1: 26744424A/T
2-1: 26783950C/G
2-1: 26801638G/A
2-1: 26831356G/A
2-1: 26902943A/G
2-1: 26997131A/G
2-1: 27032569A/G
2-1: 27085321T/A
2-1: 27125165C/T
2-1: 27157722G/A
2-1: 27172639T/C
2-1: 27188866T/C
2-1: 27223210T/G
2-1: 27289094C/T
2-1: 27339509T/A
2-1: 27366559G/C
2-1: 27389305G/C
2-1: 27425808T/C
2-1: 27479115A/G
2-1: 27492127C/T
2-1: 27492469G/T
2-1: 27504791C/T
2-1: 27529342G/A
2-1: 27543957G/A
2-1: 27544448C/A
2-1: 27545989A/G
2-1: 27545993A/G
2-1: 27545998C/T
2-1: 27546018A/T
2-1: 27564662C/T
2-1: 27615963A/T
2-1: 27711936T/C
2-1: 27765337C/T
2-1: 27861316T/G
2-1: 27935636G/T
2-1: 27961909C/T
2-1: 27996631A/G
2-1: 28026841G/T
2-1: 28064545A/T
2-1: 28096143C/T
2-1: 28177426A/T
2-1: 28214965A/C
2-1: 28216089T/A
2-1: 28216356C/T
2-1: 28216560A/C
2-1: 28230050C/A
2-1: 28243548G/T
2-1: 28300204G/A
2-1: 28325882T/C
2-1: 28417291A/C
2-1: 28430003A/G
2-1: 28486106A/G
2-1: 28523427A/T
2-1: 28544462G/A
2-1: 28604296G/T
2-1: 28767457A/T
2-1: 28774178G/A
2-1: 28814674G/A
2-1: 28827309G/C
2-1: 28861619A/C
2-1: 28952675T/A
2-1: 28978256A/T
2-1: 29059321G/A
2-1: 29105058T/C
2-1: 29123324C/T
2-1: 29181525C/T
2-1: 29194913A/G
2-1: 29236246T/A
2-1: 29237549C/G
2-1: 29274939T/G
2-1: 29306961A/T
2-1: 29450845C/T
2-1: 29469299A/G
2-1: 29492085T/A
2-1: 29498475A/T
2-1: 29520298G/A
2-1: 29533569A/C
2-1: 29564879T/C
2-1: 29568376C/T
2-1: 29588604A/T
2-1: 29668113C/A
2-1: 29683704A/T
2-1: 29723874C/G
2-1: 29753412A/G
2-1: 29820660T/A
2-1: 29821627G/A
2-1: 29834428A/T
2-1: 29867084A/G
2-1: 29898372G/C
2-1: 29991642G/T
2-1: 29999312G/A
2-1: 30001731T/C
2-1: 30012722G/T
2-1: 30084828T/C
2-1: 30166639T/C
2-1: 30468443G/C
2-1: 30509029G/A
2-1: 30544951C/A
2-1: 30582097G/T
2-1: 30639974T/A
2-1: 30743710C/T
2-1: 30789313A/G
2-1: 30894553C/A
2-1: 30952365T/C
2-1: 30973390T/C
2-1: 30984270C/T
2-1: 31268298G/A
2-1: 31278443T/C
2-1: 31297798T/C
2-1: 31328400T/C
2-1: 31356883T/C
2-1: 31425433C/T
2-1: 31456008T/G
2-1: 31516783T/C
2-1: 31628052A/G
2-1: 31638062A/C
2-1: 31700723A/G
2-1: 31747392A/G
2-1: 31842342C/T
2-1: 31906079T/A
2-1: 32001845A/G
2-1: 32031751A/G
2-1: 32031868A/G
2-1: 32037420A/G
2-1: 32037442C/A
2-1: 32057209C/T
2-1: 32131812G/A
2-1: 32158501C/T
2-1: 32196692C/T
2-1: 32207225C/T
2-1: 32248493A/T
2-1: 32281963C/T
2-1: 32335472T/A
2-1: 32366475T/C
2-1: 32420239C/T
2-1: 32453508T/C
2-1: 32453653C/A
2-1: 32473632T/C
2-1: 32476855T/G
2-1: 32477047G/T
2-1: 32477123A/G
2-1: 32507716T/A
2-1: 32511824C/T
2-1: 32539566A/G
2-1: 32549894G/A
2-1: 32592435A/G
2-1: 32603834G/A
2-1: 32689694C/A
2-1: 32695684G/T
2-1: 32716112C/G
2-1: 32737926G/A
2-1: 32826385C/G
2-1: 33005918C/A
2-1: 33041507T/C
2-1: 33050927C/T
2-1: 33051081C/T
2-1: 33051418T/C
2-1: 33174653T/C
2-1: 33189171C/A
2-1: 33240848G/A
2-1: 33445284C/A
2-1: 33667168G/A
2-1: 33758233G/A
2-1: 33868800A/T
2-1: 33900827G/A
2-1: 33924739T/A
2-1: 33927019C/T
2-1: 34040347G/A
2-1: 34087594T/C
2-1: 34141712C/G
2-1: 34178976C/A
2-1: 34233458G/A
2-1: 34269200A/G
2-1: 34287327C/T
2-1: 34397659A/C
2-1: 34585067G/A
2-1: 34750079T/A
2-1: 34813320A/G
2-1: 34813469A/G
2-1: 34814103C/T
2-1: 35101145G/A
2-1: 35168561C/T
2-1: 35196147G/C
2-1: 35409099T/C
2-1: 35435341A/T
2-1: 35566102G/T
2-1: 35588401T/C
2-1: 35638841T/A
2-1: 35727160A/G
2-1: 35736720C/G
2-1: 35736971C/T
2-1: 35745136A/G
2-1: 35776577G/A
2-1: 35789963A/T
2-1: 35806783T/C
2-1: 35850531G/A
2-1: 35876322T/C
2-1: 35968849G/A
2-1: 35986353G/A
2-1: 35996445T/A
2-1: 36014890A/C
2-1: 36031000C/T
2-1: 36109835A/G
2-1: 36125941G/A
2-1: 36134291C/A
2-1: 36182263C/T
2-1: 36208226G/A
2-1: 36252055G/A
2-1: 36270066T/C
2-1: 36273465C/T
2-1: 36303465A/G
2-1: 36359001C/T
2-1: 36359364A/T
2-1: 36383679T/A
2-1: 36407822G/A
2-1: 36501622T/G
2-1: 36582485T/A
2-1: 36700501A/G
2-1: 36787154G/A
2-1: 36827938G/C
2-1: 36907101A/G
2-1: 36969700C/T
2-1: 36977951G/A
2-1: 36980057T/G
2-1: 36998625A/G
2-1: 37012950A/T
2-1: 37155684T/C
2-1: 37463550C/G
2-1: 37474914C/T
2-1: 37485395C/T
2-1: 37496926T/C
2-1: 37582995G/T
2-1: 37624918T/C
2-1: 37687654A/G
2-1: 37739862C/T
2-1: 37793629T/C
2-1: 37935274A/C
2-1: 38001002G/T
2-1: 38023422C/T
2-1: 39547124A/T
2-1: 39547180G/A
2-1: 39547433T/C
2-1: 39547684A/G
2-1: 39558667A/G
2-1: 39593116A/G
2-1: 39678552T/C
2-1: 39798561G/A
2-1: 39908876A/C
2-1: 40024814G/A
2-1: 40062717T/C
2-1: 40098467T/A
2-1: 40125519G/C
2-1: 40278168C/T
2-1: 40310243A/T
2-1: 40359162C/T
2-1: 40572555C/A
2-1: 40582855C/T
2-1: 40611594G/A
2-1: 40614044C/T
2-1: 40691282T/C
2-1: 40717634A/T
2-1: 40825376G/A
2-1: 40848883T/C
2-1: 40875947G/C
2-1: 40929518G/A
2-1: 40940142A/G
2-1: 40941883G/A
2-1: 40955415G/A
2-1: 40957852G/A
2-1: 40958240A/T
2-1: 40966514G/A
2-1: 40971046C/T
2-1: 40999084C/T
2-1: 41108244G/A
2-1: 41141083C/T
2-1: 41156584G/A
2-1: 41182717A/T
2-1: 41209602T/A
2-1: 41256806G/A
2-1: 42056737G/T
2-1: 42067723T/C
2-1: 42080578C/T
2-1: 42096827G/A
2-1: 42143762A/T
2-1: 42206167A/G
2-1: 42309254C/T
2-1: 42418819G/A
2-1: 42439263T/C
2-1: 42571899A/T
2-1: 42599412G/A
2-1: 42627908T/C
2-1: 42658393T/G
2-1: 42658762C/G
2-1: 42658826C/T
2-1: 42667818A/G
2-1: 42756240T/C
2-1: 42865167G/T
2-1: 43008225A/T
2-1: 43056706C/T
2-1: 43057353C/T
2-1: 43059092G/T
2-1: 43059243T/C
2-1: 43059313A/T
2-1: 43128160C/T
2-1: 43139586C/T
2-1: 43157503G/A
2-1: 43228835G/A
2-1: 43251728G/A
2-1: 43278155A/G
2-1: 43381047C/A
2-1: 43385673A/C
2-1: 43440488C/T
2-1: 43470688A/T
2-1: 43540315T/A
2-1: 43576410C/T
2-1: 43593341T/C
2-1: 43598102G/A
2-1: 43641935A/G
2-1: 43653986G/A
2-1: 43672824G/A
2-1: 43706959T/C
2-1: 43734554T/C
2-1: 43762728C/T
2-1: 43780871G/A
2-1: 43839561C/A
2-1: 43898107A/T
2-1: 43933997C/T
2-1: 43939950C/T
2-1: 43946684C/T
2-1: 44067407A/G
2-1: 44067914T/A
2-1: 44067935A/G
2-1: 44068320T/G
2-1: 44083787C/T
2-1: 44101033C/A
2-1: 44112325G/A
2-1: 44159058G/A
2-1: 44165433T/G
2-1: 44165521C/A
2-1: 44189633G/A
2-1: 44240025T/A
2-1: 44317012G/A
2-1: 44345349C/A
2-1: 44361313C/A
2-1: 44460584C/T
2-1: 44470511G/T
2-1: 44473946T/C
2-1: 44530099T/C
2-1: 44542476T/G
2-1: 44674290T/A
2-1: 44792374G/T
2-1: 44792626C/T
2-1: 44918660G/C
2-1: 44935634G/C
2-1: 44967297T/G
2-1: 45016914G/A
2-1: 45051250T/C
2-1: 45147929T/A
2-1: 45157913C/T
2-1: 45160943A/C
2-1: 45252366A/T
2-1: 45267777C/G
2-1: 45383800A/G
2-1: 45501155G/A
2-1: 45557111C/A
2-1: 45635451G/A
2-1: 45638165A/.
2-1: 45640098G/A
2-1: 45655867G/T
2-1: 45686909G/C
2-1: 45756611A/G
2-1: 45790629G/A
2-1: 45810348T/A
2-1: 45874142T/A
2-1: 45882093G/A
2-1: 45884518A/G
2-1: 45896326C/T
2-1: 45907301C/T
2-1: 45951780G/T
2-1: 45953304C/T
2-1: 45953933A/G
2-1: 45954002A/T
2-1: 45983823C/A
2-1: 45985271C/T
2-1: 46015348G/A
2-1: 46082592G/A
2-1: 46086466C/T
2-1: 46088966T/A
2-1: 46089099T/C
2-1: 46089206C/T
2-1: 46107993A/G
2-1: 46125460A/G
2-1: 46155588T/C
2-1: 46283215A/G
2-1: 46322977C/T
2-1: 46338216C/T
2-1: 46360457T/A
2-1: 46375205A/T
2-1: 46376771C/G
2-1: 46493763T/C
2-1: 46538911C/T
2-1: 46570441G/A
2-1: 46624854C/G
2-1: 46698099C/A
2-1: 46698190A/G
2-1: 46736293A/G
2-1: 46778405C/T
2-1: 46888119A/T
2-1: 46900789A/G
2-1: 46948691A/G
2-1: 47007619C/T
2-1: 47062324C/T
2-1: 47081028A/T
2-1: 47141884A/G
2-1: 47218262G/T
2-1: 47245702A/T
2-1: 47247532A/T
2-1: 47295955C/G
2-1: 47342681C/T
2-1: 47389812C/A
2-1: 47432259T/A
2-1: 47950695G/T
2-1: 47992994G/A
2-1: 48028457G/A
2-1: 48053515T/C
2-1: 48080380G/A
2-1: 48109986A/G
2-1: 48151012A/G
2-1: 48161958G/A
2-1: 48255285G/A
2-1: 48256796C/A
2-1: 48408887T/A
2-1: 48723664A/G
2-1: 48805784G/A
2-1: 48940113C/T
2-1: 49291405A/T
2-1: 49302668C/A
2-1: 49342414A/G
2-1: 49425513T/A
2-1: 49467569C/T
2-1: 49519552T/A
2-1: 49521760A/G
2-1: 49582217G/A
2-1: 49615277G/C
2-1: 49691700C/A
2-1: 49721967C/T
2-1: 49741561T/C
2-1: 49743640G/C
2-1: 49855458T/A
2-1: 49896242G/A
2-1: 49940205C/T
2-1: 49956941T/C
2-1: 49982239A/G
2-1: 50013706C/A
2-1: 50038967C/A
2-1: 50078557A/T
2-1: 50115716T/A
2-1: 50197248A/G
2-1: 50221259T/G
2-1: 50233323G/T
2-1: 50235828C/T
2-1: 50264748A/G
2-1: 50317543A/T
2-1: 50317828G/C
2-1: 50318309C/T
2-1: 50349965G/C
2-1: 50410845G/A
2-1: 50443617T/C
2-1: 50470460T/C
2-1: 50480957C/T
2-1: 50480975A/G
2-1: 50494190C/G
2-1: 50505726G/A
2-1: 50530899T/A
2-1: 50597663T/C
2-1: 50602810G/T
2-1: 50633974A/G
2-1: 50700014C/T
2-1: 50730521G/A
2-1: 50775325A/T
2-1: 50799615A/G
2-1: 50819942C/T
2-1: 51020970A/G
2-1: 51199321G/A
2-1: 51199540T/A
2-1: 51201066A/G
2-1: 51303375C/T
2-1: 51324654T/C
2-1: 51332593C/T
2-1: 51332714T/A
2-1: 51495630A/T
2-1: 51646069C/A
2-1: 51688700C/T
2-1: 51704651T/C
2-1: 51706269T/C
2-1: 51721019C/A
2-1: 51725187A/G
2-1: 51728397T/A
2-1: 51752017G/T
2-1: 51813164T/G
2-1: 51908826C/T
2-1: 51943350G/C
2-1: 51943520C/G
2-1: 51953679A/C
2-1: 51979925A/G
2-1: 51988981G/A
2-1: 52005916C/A
2-1: 52059739T/G
2-1: 52182482G/A
2-1: 52262963C/A
2-1: 52312661A/T
2-1: 52346901T/A
2-1: 52365150A/C
2-1: 52514686C/T
2-1: 52631982A/T
2-1: 52654910C/T
2-1: 52708395A/T
2-1: 52769180G/T
2-1: 52805960A/T
2-1: 52872419A/G
2-1: 52946866A/C
2-1: 52963773C/T
2-1: 52985256A/T
2-1: 52990391T/C
2-1: 52998122C/T
2-1: 53012125T/A
2-1: 53012589C/T
2-1: 53012715T/G
2-1: 53027402A/G
2-1: 53061066G/C
2-1: 53111309A/T
2-1: 53123077C/T
2-1: 53141670C/T
2-1: 53141861T/C
2-1: 53149225T/C
2-1: 53153373A/T
2-1: 53193442G/A
2-1: 53242200G/A
2-1: 53243490T/C
2-1: 53257358A/T
2-1: 53275544A/G
2-1: 53301183A/G
2-1: 53327250G/A
2-1: 53361081G/A
2-1: 53414199G/A
2-1: 53453156C/T
2-1: 53490201C/T
2-1: 53507203T/C
2-1: 53528598G/A
2-1: 53545823G/A
2-1: 53561564A/T
2-1: 53575910A/G
2-1: 53596820G/A
2-1: 53633503C/G
2-1: 53666444C/A
2-1: 53682591T/A
2-1: 53693190G/T
2-1: 53732840C/A
2-1: 53825603G/T
2-1: 53861331T/A
2-1: 53862141C/T
2-1: 53871392C/A
2-1: 53924442G/A
2-1: 54003158T/G
2-1: 54152056G/A
2-1: 54181174T/A
2-1: 54276851T/C
2-1: 54284110C/A
2-1: 54319484A/T
2-1: 54331078T/A
2-1: 54365488G/A
2-1: 54459910C/T
2-1: 54536142C/T
2-1: 54568193A/T
2-1: 54600669A/T
2-1: 54607362A/G
2-1: 54610727C/A
2-1: 54613009A/G
2-1: 54613114C/T
2-1: 54613836A/T
2-1: 54625163T/G
2-1: 54658026C/T
2-1: 54694234T/C
2-1: 54723517G/A
2-1: 54790123A/C
2-1: 54819108C/T
2-1: 54873115C/T
2-1: 54935743A/C
2-1: 54984268C/T
2-1: 55004948A/T
2-1: 55005100T/A
2-1: 55005202T/C
2-1: 55026015G/A
2-1: 55062226C/T
2-1: 55103517T/A
2-1: 55118777A/G
2-1: 55135809A/C
2-1: 55171746T/C
2-1: 55176809T/G
2-1: 55205634G/A
2-1: 55217028A/G
2-1: 55267746C/A
2-1: 55306887T/G
2-1: 55384324C/T
2-1: 55398628T/C
2-1: 55411698A/C
2-1: 55435885C/T
2-1: 55495167T/A
2-1: 55499164T/C
2-1: 55504364G/A
2-1: 55532546A/G
2-1: 55533114T/C
2-1: 55568409T/G
2-1: 55677833C/T
2-1: 55692681G/A
2-1: 55757951G/A
2-1: 55966484T/G
2-1: 55966819G/C
2-1: 55967035A/G
2-1: 55967144C/T
2-1: 55967149C/T
2-1: 55967165T/C
2-1: 55967172A/T
2-1: 56137705A/G
2-1: 56194411G/A
2-1: 56256096G/A
2-1: 56278864T/C
2-1: 56292431G/T
2-1: 56299817G/A
2-1: 56300396T/A
2-1: 56300622T/G
2-1: 56300817A/T
2-1: 56301456T/A
2-1: 56301666C/A
2-1: 56301898A/G
2-1: 56302025G/A
2-1: 56302203C/G
2-1: 56302884T/C
2-1: 56302893A/G
2-1: 56303000T/C
2-1: 56303155C/T
2-1: 56303430G/A
2-1: 56303597T/A
2-1: 56304056G/A
2-1: 56304230T/C
2-1: 56304415A/G
2-1: 56313122A/G
2-1: 56315088T/A
2-1: 56315434G/T
2-1: 56315965A/T
2-1: 56316573G/A
2-1: 56317926G/A
2-1: 56327047G/C
2-1: 56327185T/C
2-1: 56327594G/A
2-1: 56327872A/G
2-1: 56328044C/T
2-1: 56328098G/A
2-1: 56328108G/A
2-1: 56346627C/T
2-1: 56384370A/G
2-1: 56434985G/A
2-1: 56455644G/A
2-1: 56476683A/C
2-1: 56483679G/A
2-1: 56543572A/G
2-1: 56737646A/C
2-1: 56740519T/C
2-1: 56740770A/G
2-1: 56740873T/C
2-1: 56743141A/G
2-1: 56755255T/G
2-1: 56774353T/C
2-1: 56849123C/T
2-1: 56879766T/G
2-1: 57016436T/A
2-1: 57030859T/C
2-1: 57123904G/A
2-1: 57136233G/A
2-1: 57216617C/G
2-1: 57246871A/G
2-1: 57292180G/A
2-1: 57322865C/A
2-1: 57329394A/T
2-1: 57368483C/T
2-1: 57369570C/T
2-1: 57387161A/G
2-1: 57399520G/A
2-1: 57471522T/C
2-1: 57524957T/C
2-1: 57591490C/G
2-1: 57666180A/G
2-1: 57715209C/T
2-1: 57771644T/G
2-1: 57786471G/T
2-1: 57867386A/C
2-1: 57933673A/T
2-1: 57958860T/C
2-1: 57973399A/G
2-1: 57984655C/T
2-1: 58055243T/C
2-1: 58116645T/A
2-1: 58149392A/G
2-1: 58216844T/A
2-1: 58266003T/A
2-1: 58281915T/C
2-1: 58330043G/A
2-1: 58353755A/T
2-1: 58459117C/A
2-1: 58545701G/A
2-1: 58577554T/C
2-1: 58650470A/G
2-1: 58680972T/C
2-1: 58729926T/C
2-1: 58769444C/A
2-1: 58821868G/T
2-1: 58872674G/A
2-1: 58929080T/C
2-1: 58934716C/G
2-1: 58936053C/A
2-1: 58952686A/C
2-1: 58953217A/G
2-1: 58953238A/C
2-1: 58974969A/G
2-1: 58992880G/A
2-1: 59034334T/G
2-1: 59056832T/C
2-1: 59137879T/A
2-1: 59192880A/G
2-1: 59247509G/A
2-1: 59295416A/C
2-1: 59311287A/G
2-1: 59316983C/T
2-1: 59351560G/A
2-1: 59414159G/A
2-1: 59414259G/T
2-1: 59418224C/T
2-1: 59478153A/T
2-1: 59478271G/C
2-1: 59542071A/C
2-1: 59606097G/T
2-1: 59772274C/T
2-1: 60021874T/A
2-1: 60099926C/T
2-1: 60202669C/T
2-1: 60275497G/A
2-1: 60293804T/C
2-1: 60371574T/A
2-1: 60382974G/A
2-1: 60473267A/G
2-1: 60546619G/T
2-1: 60553132C/A
2-1: 60557779A/T
2-1: 60586832C/A
2-1: 60600730C/T
2-1: 60641960G/A
2-1: 60664343A/C
2-1: 60681063C/T
2-1: 60684453G/A
2-1: 60684732C/T
2-1: 60684777C/T
2-1: 60684852C/G
2-1: 60685023G/A
2-1: 60703567C/T
2-1: 60741866A/T
2-1: 60858319A/G
2-1: 60967468G/A
2-1: 60968000G/A
2-1: 61136913G/A
2-1: 61332225A/C
2-1: 61357666C/A
2-1: 61479787A/G
2-1: 61702589G/T
2-1: 61779657C/A
2-1: 61823411A/T
2-1: 61871398T/C
2-1: 61925602G/A
2-1: 61975066T/C
2-1: 61990917G/A
2-1: 61994610G/A
2-1: 62002045C/T
2-1: 62167825C/T
2-1: 62176905C/T
2-1: 62241452T/G
2-1: 62434908T/G
2-1: 62572129T/C
2-1: 62611905A/G
2-1: 62642671C/T
2-1: 62654058C/T
2-1: 62806863T/A
2-1: 62806906A/.
2-1: 62821456C/T
2-1: 62890315T/C
2-1: 62902246A/C
2-1: 62920541G/T
2-1: 62973071T/G
2-1: 62996284G/T
2-1: 63064562C/G
2-1: 63135798C/T
2-1: 63165161A/G
2-1: 63212650A/C
2-1: 63295623T/C
2-1: 63331849G/C
2-1: 63437074C/T
2-1: 63547889C/G
2-1: 63565098A/T
2-1: 63576139A/G
2-1: 63679390C/T
2-1: 63789530A/G
2-1: 63962086C/T
2-1: 63997156T/C
2-1: 64045704T/A
2-1: 64129378C/A
2-1: 64131160A/T
2-1: 64142698A/G
2-1: 64143434A/T
2-1: 64182599G/T
2-1: 64231986C/G
2-1: 64232972C/A
2-1: 64236856C/T
2-1: 64236877T/C
2-1: 64248599T/C
2-1: 64251465A/G
2-1: 64286075A/G
2-1: 64323232A/C
2-1: 64338652A/G
2-1: 64338736A/G
2-1: 64338822C/.
2-1: 64338933T/.
2-1: 64355837C/T
2-1: 64442085A/C
2-1: 64500957C/A
2-1: 64695664C/T
2-1: 64697827G/A
2-1: 64697897C/T
2-1: 64697926G/T
2-1: 64697945C/T
2-1: 64697964G/A
2-1: 64700381G/A
2-1: 64705238G/A
2-1: 64709778C/A
2-1: 64747801G/A
2-1: 64776300C/A
2-1: 64804336C/T
2-1: 64863008G/A
2-1: 64899904C/T
2-1: 64977186A/G
2-1: 65043836G/A
2-1: 65046062G/C
2-1: 65067482C/T
2-1: 65093526T/C
2-1: 65239010A/G
2-1: 65247823G/A
2-1: 65265000A/G
2-1: 65413482G/A
2-1: 65435547A/G
2-1: 65437688C/T
2-1: 65437867G/A
2-1: 65437988T/G
2-1: 65438098C/T
2-1: 65438128G/C
2-1: 65438174G/T
2-1: 65458522T/A
2-1: 65487617T/C
2-1: 65580544T/C
2-1: 65582166T/C
2-1: 65660800G/A
2-1: 65861624A/T
2-1: 65861651C/T
2-1: 65861695A/G
2-1: 65861706A/G
2-1: 65861724G/T
2-1: 65861741G/A
2-1: 65861751C/G
2-1: 65900474G/A
2-1: 65978445C/T
2-1: 66051786C/A
2-1: 66109947C/T
2-1: 66173377T/C
2-1: 66242545A/G
2-1: 66293511T/C
2-1: 66337462T/A
2-1: 66459520T/G
2-1: 66476697A/G
2-1: 66490061C/T
2-1: 66510023C/G
2-1: 66545641A/G
2-1: 66590958A/C
2-1: 66647780T/A
2-1: 66681505C/T
2-1: 66781170A/G
2-1: 66835590G/T
2-1: 66847726A/T
2-1: 66875440C/T
2-1: 67002149A/T
2-1: 67011577G/A
2-1: 67111664C/T
2-1: 67150890C/A
2-1: 67237021A/G
2-1: 67327078C/T
2-1: 67331662T/C
2-1: 67348517T/G
2-1: 67358697C/T
2-1: 67372165G/A
2-1: 67453855C/T
2-1: 67505344A/G
2-1: 67516415G/C
2-1: 67607512G/A
2-1: 67626542G/A
2-1: 67640940T/A
2-1: 67646213C/T
2-1: 67663901T/A
2-1: 67663938T/G
2-1: 67689156A/G
2-1: 67781522C/T
2-1: 67887677G/A
2-1: 67906456A/G
2-1: 67923715C/T
2-1: 68002834A/C
2-1: 68090839T/G
2-1: 68099749G/T
2-1: 68154996G/T
2-1: 68227510T/C
2-1: 68275886T/A
2-1: 68290083G/A
2-1: 68374880C/G
2-1: 68377982G/A
2-1: 68431060C/G
2-1: 68443809C/T
2-1: 68505560T/A
2-1: 68506683A/G
2-1: 68506854G/A
2-1: 68513532A/G
2-1: 68516301A/G
2-1: 68524686C/G
2-1: 68524809T/C
2-1: 68525575C/T
2-1: 68525707C/A
2-1: 68525819T/C
2-1: 68543273T/G
2-1: 68586960A/G
2-1: 68602304G/A
2-1: 68657066A/G
2-1: 68787186T/A
2-1: 68845354T/A
2-1: 68884612A/T
2-1: 68920761T/A
2-1: 68968822T/A
2-1: 68988918C/A
2-1: 69007909A/T
2-1: 69033124A/G
2-1: 69062794T/A
2-1: 69088374A/C
2-1: 69100748G/A
2-1: 69166672C/A
2-1: 69208298C/T
2-1: 69290308C/T
2-1: 69340507C/T
2-1: 69406091G/T
2-1: 69510700G/A
2-1: 69563242C/G
2-1: 69717006A/G
2-1: 69771373T/A
2-1: 69780764C/T
2-1: 69800605C/T
2-1: 69868877G/T
2-1: 69922486T/A
2-1: 70043197C/T
2-1: 70191463C/A
2-1: 70210120T/A
2-1: 70243595T/C
2-1: 70310107C/T
2-1: 70340983T/C
2-1: 70424720T/G
2-1: 70561284G/A
2-1: 70622040T/A
2-1: 70736719T/C
2-1: 70845263G/A
2-1: 70939204C/T
2-1: 71010750T/G
2-1: 71153513G/T
2-1: 71195322T/C
2-1: 71210186G/A
2-1: 71291477T/A
2-1: 71424028C/T
2-1: 71436447T/C
2-1: 71500327A/G
2-1: 71532645G/T
2-1: 71558464G/A
2-1: 71607606G/A
2-1: 71689141C/T
2-1: 71749125A/T
2-1: 71791429A/G
2-1: 71829072G/A
2-1: 71871633T/C
2-1: 71882097C/T
2-1: 71915359T/C
2-1: 71932415A/G
2-1: 72011572A/G
2-1: 72054920G/A
2-1: 72103173G/A
2-1: 72137500T/C
2-1: 72141569A/T
2-1: 72201059C/A
2-1: 72267641A/T
2-1: 72318923C/T
2-1: 72337111C/T
2-1: 72454540T/C
2-1: 72478387C/T
2-1: 72511183A/T
2-1: 72562681G/A
2-1: 72577624A/T
2-1: 72765077C/G
2-1: 72814235G/C
2-1: 72987752T/C
2-1: 73051263G/A
2-1: 73098056G/A
2-1: 73125293C/A
2-1: 73161262A/G
2-1: 73162743A/C
2-1: 73231887T/C
2-1: 73305414T/C
2-1: 73326911G/A
2-1: 73394497A/G
2-1: 73426398T/G
2-1: 73541005G/A
2-1: 73542813G/A
2-1: 73677069C/T
2-1: 73823843T/C
2-1: 73999399T/C
2-1: 74137692A/C
2-1: 74190897C/T
2-1: 74239413C/T
2-1: 74361545C/A
2-1: 74461469T/A
2-1: 74472309T/C
2-1: 74475434T/A
2-1: 74479073C/G
2-1: 74479185C/T
2-1: 74479317A/T
2-1: 74481748C/A
2-1: 74484984A/T
2-1: 74496267T/C
2-1: 74597168A/T
2-1: 74680690C/G
2-1: 74712711C/A
2-1: 74722793G/A
2-1: 74745779A/C
2-1: 74823516A/C
2-1: 74873820G/A
2-1: 74943322G/T
2-1: 75075856A/G
2-1: 75131086C/T
2-1: 75251578G/A
2-1: 75409634T/C
2-1: 75461892T/A
2-1: 75489310T/C
2-1: 75534152C/G
2-1: 75672992C/T
2-1: 75683185G/T
2-1: 75774113T/A
2-1: 75852084A/G
2-1: 75884306A/T
2-1: 75886075T/C
2-1: 75973623A/G
2-1: 76115845A/T
2-1: 76275321C/T
2-1: 76276468C/T
2-1: 76542848G/A
2-1: 76545535C/T
2-1: 76620289T/A
2-1: 76630983T/C
2-1: 76663124G/A
2-1: 76673483C/A
2-1: 76739641G/C
2-1: 76769916T/C
2-1: 76820620C/T
2-1: 76871225T/C
2-1: 76879707G/A
2-1: 76898717G/A
2-1: 76924130A/T
2-1: 76984059A/G
2-1: 77073386C/T
2-1: 77271449G/A
2-1: 77274310A/C
2-1: 77285183G/T
2-1: 77286989C/G
2-1: 77339951A/C
2-1: 77417669G/A
2-1: 77689794T/G
2-1: 77864125G/A
2-1: 78038481A/C
2-1: 78124399C/A
2-1: 78126335C/T
2-1: 78156801C/T
2-1: 78310387A/T
2-1: 78311585G/T
2-1: 78373274A/C
2-1: 78518578G/A
2-1: 78545565C/T
2-1: 78686840G/C
2-1: 79060255A/C
2-1: 79093138G/A
2-1: 79155680G/A
2-1: 79277912C/T
2-1: 79302219T/A
2-1: 79568885C/T
2-1: 79672014C/T
2-1: 79674275G/A
2-1: 79907312A/G
2-2: 10795T/A
2-2: 82877T/C
2-2: 762285C/A
2-2: 841987C/A
2-2: 1111006T/C
2-2: 1128269G/T
2-2: 1135007G/A
2-2: 1157864T/C
2-2: 1263637T/G
2-2: 1437813C/T
2-2: 1521395C/T
2-2: 1630474A/T
2-2: 1779845G/C
2-2: 1791484C/T
2-2: 1806288T/C
2-2: 1845171C/T
2-2: 2260606A/G
2-2: 2524853A/C
2-2: 2707883C/T
2-2: 2739417T/G
2-2: 3062097G/A
2-2: 3062128A/G
2-2: 3062139T/A
2-2: 3062268C/A
2-2: 3062299T/C
2-2: 3062302A/T
2-2: 3062411C/A
2-2: 3062526C/T
2-2: 3062659G/A
2-2: 3062802C/A
2-2: 3062902C/T
2-2: 3063161C/T
2-2: 3063236A/G
2-2: 3063348C/T
2-2: 3063554C/T
2-2: 3063657A/C
2-2: 3064082A/T
2-2: 3064181C/T
2-2: 3064310C/T
2-2: 3064350T/A
2-2: 3064456G/T
2-2: 3064709A/C
2-2: 3064793A/G
2-2: 3079392A/C
2-2: 3107302A/G
2-2: 3193160T/C
2-2: 3258756T/C
2-2: 3327644G/T
2-2: 3478313T/G
2-2: 3545073T/A
2-2: 3608668G/A
2-2: 3619185C/T
2-2: 3684494G/A
2-2: 3776460G/A
2-2: 3785378T/C
2-2: 3807181T/C
2-2: 3881598A/G
2-2: 3896849G/C
2-2: 3958779T/A
2-2: 4004085A/G
2-2: 4029611A/C
2-2: 4045430T/A
2-2: 4050172A/G
2-2: 4063320G/A
2-2: 4084212C/A
2-2: 4097503A/T
2-2: 4119981C/A
2-2: 4146972A/G
2-2: 4230082G/A
2-2: 4427674A/C
2-2: 4444635A/C
2-2: 4460333G/C
2-2: 4481525A/G
2-2: 4483066C/A
2-2: 4483709A/G
2-2: 4498749T/C
2-2: 4545617G/C
2-2: 4571018A/C
2-2: 4580076A/G
2-2: 4609791C/T
2-2: 4712751A/G
2-2: 4763544G/A
2-2: 4797285A/G
2-2: 4801505A/G
2-2: 4813806A/G
2-2: 4838744A/G
2-2: 4838792T/C
2-2: 4841388T/C
2-2: 4860997A/T
2-2: 4961688A/T
2-2: 4964030A/C
2-2: 4987896C/T
2-2: 5046212T/C
2-2: 5097979C/T
2-2: 5204059G/A
2-2: 5214529A/G
2-2: 5346999G/A
2-2: 5404532G/T
2-2: 5434485T/C
2-2: 5454464T/C
2-2: 5465158C/G
2-2: 5476572C/A
2-2: 5507655A/T
2-2: 5558538A/G
2-2: 5741163C/T
2-2: 5753878A/G
2-2: 5791492C/A
2-2: 5818430G/T
2-2: 5894205A/T
2-2: 5943698C/A
2-2: 5945211G/T
2-2: 5945213C/A
2-2: 5946881C/T
2-2: 5954764C/G
2-2: 5955246G/A
2-2: 5956271T/C
2-2: 6036889T/C
2-2: 6050591G/A
2-2: 6059802C/T
2-2: 6060878C/T
2-2: 6062304C/T
2-2: 6090946G/C
2-2: 6179977T/A
2-2: 6212041G/T
2-2: 6222234G/T
2-2: 6238324A/C
2-2: 6246085C/G
2-2: 6247676C/T
2-2: 6273003T/C
2-2: 6330251G/A
2-2: 6354543G/A
2-2: 6462267G/A
2-2: 6495333C/G
2-2: 6538637C/T
2-2: 6624272A/G
2-2: 6738735T/A
2-2: 6759759T/A
2-2: 6908034T/A
2-2: 6995094G/A
2-2: 7094050C/T
2-2: 7147198T/C
2-2: 7263327G/T
2-2: 7375047A/G
2-2: 7405673A/T
2-2: 7489421T/C
2-2: 7529546A/T
2-2: 7539928G/T
2-2: 7608048G/A
2-2: 7711887G/A
2-2: 7715063T/C
2-2: 7742059T/C
2-2: 7804876G/C
2-2: 7877916A/G
2-2: 7905833G/A
2-2: 8023464T/A
2-2: 8023818C/T
2-2: 8024581C/T
2-2: 8045187C/T
2-2: 8072374T/C
2-2: 8120666T/G
2-2: 8138704T/C
2-2: 8168176T/A
2-2: 8217594A/G
2-2: 8296166G/A
2-2: 8316025C/A
2-2: 8316200C/T
2-2: 8316313T/C
2-2: 8341783C/A
2-2: 8452921C/T
2-2: 8483432A/G
2-2: 8610218G/C
2-2: 8662520C/T
2-2: 8675867T/C
2-2: 8687994T/A
2-2: 8792035A/C
2-2: 8848716T/A
2-2: 8964445A/T
2-2: 9029512T/A
2-2: 9071400A/G
2-2: 9138097T/A
2-2: 9294636G/T
2-2: 9330477A/T
2-2: 9377362A/C
2-2: 9405681T/C
2-2: 9627419A/G
2-2: 9659722C/A
2-2: 9799184G/A
2-2: 9872669C/A
2-2: 9920007G/A
2-2: 9974594C/T
2-2: 10100395T/C
2-2: 10193787A/T
2-2: 10194982A/G
2-2: 10204075G/A
2-2: 10235602A/G
2-2: 10310883A/T
2-2: 10342423C/T
2-2: 10344973C/T
2-2: 10446834A/T
2-2: 10483191A/G
2-2: 10570658T/C
2-2: 10643809G/T
2-2: 10752060C/T
2-2: 10764123A/G
2-2: 10880218C/T
2-2: 10915688C/G
2-2: 11006612G/A
2-2: 11051680C/T
2-2: 11087711A/G
2-2: 11172843C/T
2-2: 11198626C/A
2-2: 11266849C/T
2-2: 11306728T/G
2-2: 11400129A/G
2-2: 11471029G/A
2-2: 11546583C/T
2-2: 11598134G/A
2-2: 11640311C/T
2-2: 11679788C/T
2-2: 11713805C/T
2-2: 11746643A/G
2-2: 11761437A/G
2-2: 11796602C/T
2-2: 11817458C/T
2-2: 11970965A/T
2-2: 11997473T/C
2-2: 12052126T/C
2-2: 12139975A/G
2-2: 12303245G/T
2-2: 12305170G/A
2-2: 12372497G/C
2-2: 12394784A/C
2-2: 12414059A/G
2-2: 12424794G/A
2-2: 12452518T/C
2-2: 12491101C/G
2-2: 12516725A/C
2-2: 12615203A/G
2-2: 12652402T/C
2-2: 12738940C/T
2-2: 12850921C/T
2-2: 12939060G/A
2-2: 13079609A/G
2-2: 13087023A/G
2-2: 13099568C/T
2-2: 13100412G/T
2-2: 13198247G/T
2-2: 13219669A/G
2-2: 13237165T/G
2-2: 13274410C/T
2-2: 13349461A/G
2-2: 13441977G/A
2-2: 13491542C/G
2-2: 13521364A/G
2-2: 13547851G/A
2-2: 13577939G/T
2-2: 13608627G/A
2-2: 13635302G/C
2-2: 13678225T/C
2-2: 13722001T/C
2-2: 13808206C/A
2-2: 13846715T/C
2-2: 13911622C/A
2-2: 13948302C/T
2-2: 13948326T/A
2-2: 13948757G/A
2-2: 13977580C/T
2-2: 13998583G/C
2-2: 14041137A/T
2-2: 14042312A/G
2-2: 14042380C/A
2-2: 14056042A/G
2-2: 14084141T/C
2-2: 14131226T/A
2-2: 14157205C/T
2-2: 14213042G/A
2-2: 14410171A/G
2-2: 14720340A/C
2-2: 14834622C/T
2-2: 14870772T/C
2-2: 14977898G/A
2-2: 15022491G/C
2-2: 15045719A/G
2-2: 15161851A/C
2-2: 15380964G/A
2-2: 15428959A/G
2-2: 15664254G/T
2-2: 15796059T/C
2-2: 15829952T/C
2-2: 15865521A/C
2-2: 15956126A/C
2-2: 15984753G/A
2-2: 16008629G/A
2-2: 16019828T/C
2-2: 16034672A/G
2-2: 16138828C/A
2-2: 16139837G/A
2-2: 16142790C/T
2-2: 16183758T/A
2-2: 16235604A/T
2-2: 16296922A/T
2-2: 16368181T/A
2-2: 16430753A/G
2-2: 16457244C/T
2-2: 16486285A/G
2-2: 16494396A/T
2-2: 16539207T/C
2-2: 16618909G/C
2-2: 16652307G/T
2-2: 16709070C/G
2-2: 16760314C/T
2-2: 16772045G/T
2-2: 16772982C/G
2-2: 16773082C/T
2-2: 16799248C/G
2-2: 16841780C/T
2-2: 16845998T/A
2-2: 16867569C/G
2-2: 16894989G/A
2-2: 16903883T/.
2-2: 16903910T/.
2-2: 16904044T/.
2-2: 16904362G/C
2-2: 16904420C/G
2-2: 16904456G/A
2-2: 16916176A/T
2-2: 16926446A/G
2-2: 16926564T/C
2-2: 16927148A/G
2-2: 16954714T/G
2-2: 16974150A/T
2-2: 17086281A/T
2-2: 17119025A/G
2-2: 17181169G/C
2-2: 17225455T/C
2-2: 17278453T/A
2-2: 17303030T/A
2-2: 17378702A/C
2-2: 17427493A/C
2-2: 17481477A/G
2-2: 17496909T/C
2-2: 17553481G/A
2-2: 17634483G/C
2-2: 17691044T/C
2-2: 17800047G/A
2-2: 17854695C/T
2-2: 17869392A/T
2-2: 17880837T/C
2-2: 17897426A/G
2-2: 17926418G/T
2-2: 17974645A/T
2-2: 18014819T/A
2-2: 18202568C/G
2-2: 18246987C/T
2-2: 18329394A/C
2-2: 18371847A/C
2-2: 18379298C/G
2-2: 18455097T/A
2-2: 18483178C/A
2-2: 18528337T/C
2-2: 18570574A/C
2-2: 18575441G/A
2-2: 18605444G/T
2-2: 18640141G/A
2-2: 18729217G/A
2-2: 18732385C/A
2-2: 18849614G/T
2-2: 18875394T/A
2-2: 18905991T/C
2-2: 18977512A/G
2-2: 18995888G/A
2-2: 19089756A/G
2-2: 19166314T/C
2-2: 19284683G/T
2-2: 19379710C/A
2-2: 19413267T/C
2-2: 19455225G/T
2-2: 19484531T/A
2-2: 19505444T/A
2-2: 19555843T/G
2-2: 19593702A/G
2-2: 19594314G/T
2-2: 19596106A/G
2-2: 19615490T/A
2-2: 19709867T/C
2-2: 19741229A/G
2-2: 19759628C/T
2-2: 19808272A/G
2-2: 19895731A/C
2-2: 19896024G/A
2-2: 19896185C/A
2-2: 19896481G/A
2-2: 19896561A/T
2-2: 19898022C/G
2-2: 19898209A/G
2-2: 19898214T/A
2-2: 19898406G/C
2-2: 19898507G/A
2-2: 19909618C/A
2-2: 19909741T/C
2-2: 19909867T/C
2-2: 19910025G/A
2-2: 19910115C/T
2-2: 19910138T/A
2-2: 19910241T/G
2-2: 19910413G/A
2-2: 19910830C/T
2-2: 19911028C/T
2-2: 19911120G/C
2-2: 19911136C/A
2-2: 19911209A/T
2-2: 19911427T/C
2-2: 19911573G/T
2-2: 19911683G/T
2-2: 19911715C/G
2-2: 19911833G/A
2-2: 19911934A/G
2-2: 19912133A/G
2-2: 19914044C/T
2-2: 19914100A/G
2-2: 19914219T/A
2-2: 19914336A/G
2-2: 19914495G/T
2-2: 19914614C/T
2-2: 19914769G/A
2-2: 19915089C/A
2-2: 19915266C/T
2-2: 19915388C/T
2-2: 19915591T/G
2-2: 19916320C/A
2-2: 19925524T/G
2-2: 19968358G/A
2-2: 19993215T/A
2-2: 19993351C/A
2-2: 19993538C/G
2-2: 19993539A/T
2-2: 20017542G/A
2-2: 20017573C/T
2-2: 20017586C/.
2-2: 20017616C/A
2-2: 20078055C/T
2-2: 20125356C/T
2-2: 20165371C/T
2-2: 20193937T/A
2-2: 20207489T/C
2-2: 20233037T/C
2-2: 20273662A/T
2-2: 20364170C/T
2-2: 20420999A/G
2-2: 20477378C/T
2-2: 20532976C/A
2-2: 20565625G/A
2-2: 20586683C/G
2-2: 20639613C/A
2-2: 20675309A/G
2-2: 20686207G/C
2-2: 20776861A/G
2-2: 20786686A/G
2-2: 20795510T/G
2-2: 20795623A/T
2-2: 20795816C/G
2-2: 20796324C/T
2-2: 20809568G/T
2-2: 20824678A/G
2-2: 20924758G/C
2-2: 20931268A/G
2-2: 20944947A/C
2-2: 20957173T/C
2-2: 21032640C/T
2-2: 21146683C/A
2-2: 21344837C/G
2-2: 21416091G/A
2-2: 21471212A/G
2-2: 21513941G/A
2-2: 21551534T/A
2-2: 21555652A/T
2-2: 21556150T/A
2-2: 21570956T/G
2-2: 21574827C/T
2-2: 21574935T/C
2-2: 21598684T/G
2-2: 21642441C/T
2-2: 21687033T/C
2-2: 21734282A/G
2-2: 21767761A/T
2-2: 21790905T/G
2-2: 21792914A/G
2-2: 21833474T/C
2-2: 21916135C/T
2-2: 21972231A/G
2-2: 22113924G/A
2-2: 22157972G/T
2-2: 22214613G/T
2-2: 22225453T/C
2-2: 22319006T/C
2-2: 22331963A/G
2-2: 22447182C/T
2-2: 22459199G/A
2-2: 22484555C/A
2-2: 22484774T/G
2-2: 22485438C/T
2-2: 22500490G/C
2-2: 22507168C/G
2-2: 22507616G/A
2-2: 22507978G/A
2-2: 22508409A/G
2-2: 22509124C/T
2-2: 22511034C/T
2-2: 22520407C/G
2-2: 22525802T/C
2-2: 22527668T/C
2-2: 22528154T/C
2-2: 22528375C/G
2-2: 22541908A/G
2-2: 22560135C/T
2-2: 22560819T/C
2-2: 22584721G/A
2-2: 22590978A/G
2-2: 22590981A/T
2-2: 22592201T/G
2-2: 22592669C/T
2-2: 22593489C/T
2-2: 22594004G/A
2-2: 22594124C/T
2-2: 22628669A/C
2-2: 22672304A/G
2-2: 22740585G/A
2-2: 22764164T/C
2-2: 22778025C/G
2-2: 22877320T/C
2-2: 22879957A/T
2-2: 22927850T/C
2-2: 22956258C/T
2-2: 23019533A/G
2-2: 23086078T/C
2-2: 23099386G/A
2-2: 23099492G/T
2-2: 23106964T/G
2-2: 23107335C/A
2-2: 23107361A/G
2-2: 23107470G/T
2-2: 23108381G/A
2-2: 23108383A/G
2-2: 23108439G/A
2-2: 23108581G/T
2-2: 23108699T/C
2-2: 23108849A/C
2-2: 23108900A/C
2-2: 23108965A/G
2-2: 23109134T/A
2-2: 23109246G/A
2-2: 23109365G/A
2-2: 23109546T/G
2-2: 23109774C/T
2-2: 23110008C/T
2-2: 23110010A/G
2-2: 23110135A/G
2-2: 23110303C/T
2-2: 23175374A/G
2-2: 23180070T/A
2-2: 23197001G/A
2-2: 23291882C/T
2-2: 23305982T/A
2-2: 23320368A/C
2-2: 23405806G/A
2-2: 23422865C/A
2-2: 23497872G/C
2-2: 23514347G/A
2-2: 23562086T/A
2-2: 23693078T/A
2-2: 23714887A/G
2-2: 23801276G/A
2-2: 23811545G/T
2-2: 23862864T/C
2-2: 23879628C/T
2-2: 23908064G/T
2-2: 24017089C/G
2-2: 24034204C/T
2-2: 24314194T/G
2-2: 24315370C/T
2-2: 24333657G/C
2-2: 24344643A/G
2-2: 24456467T/C
2-2: 24458986G/A
2-2: 24525435C/T
2-2: 24563065C/T
2-2: 24594461C/T
2-2: 24615152A/T
2-2: 24881054A/T
2-2: 24971489A/G
2-2: 24999441G/A
2-2: 25073952C/A
2-2: 25077894A/G
2-2: 25092280A/C
2-2: 25105506C/T
2-2: 25210781C/T
2-2: 25211037A/T
2-2: 25213255T/C
2-2: 25213455C/T
2-2: 25246608A/G
2-2: 25258374A/G
2-2: 25271260A/C
2-2: 25271676G/A
2-2: 25273435C/G
2-2: 25273623A/G
2-2: 25273646C/T
2-2: 25287451A/G
2-2: 25380001C/T
2-2: 25441144C/T
2-2: 25470037C/T
2-2: 25553400T/G
2-2: 25557817A/G
2-2: 25561670C/T
2-2: 25581374A/G
2-2: 25606514T/A
2-2: 25614819A/G
2-2: 25625092T/C
2-2: 25626050C/T
2-2: 25626615A/G
2-2: 25626719A/G
2-2: 25630974A/T
2-2: 25636152A/G
2-2: 25661881C/G
2-2: 25768672A/C
2-2: 25816337G/A
2-2: 25853680T/A
2-2: 25853878T/C
2-2: 25853884G/T
2-2: 25855940T/A
2-2: 25855942A/T
2-2: 25855952A/T
2-2: 25856386T/A
2-2: 25857606T/G
2-2: 25857743G/A
2-2: 25858230G/T
2-2: 25858237T/A
2-2: 26030165G/A
2-2: 26076442T/C
2-2: 26142202T/A
2-2: 26152286A/G
2-2: 26180950C/T
2-2: 26206966G/A
2-2: 26235926A/G
2-2: 26262420T/C
2-2: 26311129C/A
2-2: 26341486G/T
2-2: 26425555G/A
2-2: 26452115T/C
2-2: 26490951T/A
2-2: 26592430G/C
2-2: 26715669G/C
2-2: 26746900C/A
2-2: 26808750A/G
2-2: 26825423G/A
2-2: 26867989G/A
2-2: 26908672C/T
2-2: 26940570T/C
2-2: 27016583G/T
2-2: 27309251C/T
2-2: 27362483A/G
2-2: 27381300C/T
2-2: 27430471C/T
2-2: 27449868C/A
2-2: 27615101C/G
2-2: 27623127A/T
2-2: 27626372C/T
2-2: 27655128G/A
2-2: 27658600G/A
2-2: 27684356C/T
2-2: 27812279G/A
2-2: 27827164A/T
2-2: 27840515T/C
2-2: 27862269T/C
2-2: 27891397T/G
2-2: 27925573A/T
2-2: 28039601A/C
2-2: 28050142G/A
2-2: 28102173G/A
2-2: 28162561C/G
2-2: 28165536C/T
2-2: 28181897A/G
2-2: 28182943A/G
2-2: 28260551A/T
2-2: 28293721A/G
2-2: 28311986C/T
2-2: 28329945A/G
2-2: 28353298A/C
2-2: 28367437G/A
2-2: 28410920G/A
2-2: 28482579C/T
2-2: 28557675C/T
2-2: 28651426A/G
2-2: 28689117C/A
2-2: 28757574C/A
2-2: 28902343T/C
2-2: 28946995A/T
2-2: 28989345T/C
2-2: 29005191T/C
2-2: 29036514T/C
2-2: 29070169C/A
2-2: 29085053A/C
2-2: 29149125T/A
2-2: 29161897A/T
2-2: 29175235A/G
2-2: 29199516A/T
2-2: 29230033T/C
2-2: 29256633T/C
2-2: 29318828A/G
2-2: 29353553G/C
2-2: 29373527A/G
2-2: 29389337C/T
2-2: 29403267C/T
2-2: 29417223G/A
2-2: 29511494G/T
2-2: 29511980C/A
2-2: 29512030A/T
2-2: 29531938A/T
2-2: 29551082T/G
2-2: 29655317G/A
2-2: 29817666G/A
2-2: 29820512A/C
2-2: 29854243C/T
2-2: 29870276G/A
2-2: 29889625A/T
2-2: 29912185T/G
2-2: 29925888A/G
2-2: 29962874G/C
2-2: 30023915G/T
2-2: 30091853A/T
2-2: 30148158A/C
2-2: 30167074T/C
2-2: 30167292T/C
2-2: 30208119T/A
2-2: 30248528T/C
2-2: 30286338A/G
2-2: 30344067G/C
2-2: 30391735T/C
2-2: 30515419C/T
2-2: 30548578T/A
2-2: 30731664C/T
2-2: 30814329C/T
2-2: 30937736A/T
2-2: 31081464C/T
2-2: 31169470T/C
2-2: 31497786A/G
2-2: 31606426T/G
2-2: 31695044G/A
2-2: 31733929C/T
2-2: 31747980T/C
2-2: 31877368A/T
2-2: 31981678T/C
2-2: 32098022C/T
2-2: 32110081T/C
2-2: 32211043A/G
2-2: 32262761A/T
2-2: 32302505T/C
2-2: 32338118A/G
2-2: 32410942T/G
2-2: 32444163A/T
2-2: 32493417A/G
2-2: 32493420G/T
2-2: 32493942A/T
2-2: 32585942G/A
2-2: 32617128C/T
2-2: 32642449T/A
2-2: 32711331G/A
2-2: 32711410G/A
2-2: 32732745T/C
2-2: 32890603T/A
2-2: 32942732G/T
2-2: 33039238T/C
2-2: 33078105C/T
2-2: 33105920T/C
2-2: 33219940C/T
2-2: 33267855G/T
2-2: 33293870G/T
2-2: 33293982C/T
2-2: 33294136C/A
2-2: 33294538A/G
2-2: 33294942C/A
2-2: 33295312A/G
2-2: 33313971A/G
2-2: 33331202G/A
2-2: 33344668C/T
2-2: 33521918T/G
2-2: 33581577T/C
2-2: 33655953A/G
2-2: 33730175C/G
2-2: 33740278A/G
2-2: 33764052G/C
2-2: 33825646C/T
2-2: 33857368C/T
2-2: 33897565C/T
2-2: 33910069T/G
2-2: 33910455A/G
2-2: 33922650G/A
2-2: 34104801A/G
2-2: 34165375A/T
2-2: 34223013T/C
2-2: 34409807C/T
2-2: 34499484G/A
2-2: 34579470G/A
2-2: 34720918A/T
2-2: 34888245G/A
2-2: 34946929T/G
2-2: 34985485G/A
2-2: 35066981C/T
2-2: 35115750T/C
2-2: 35126590C/T
2-2: 35126811T/A
2-2: 35127803C/A
2-2: 35147018A/T
2-2: 35148729G/A
2-2: 35148839C/T
2-2: 35166471C/A
2-2: 35238000G/C
2-2: 35292650A/T
2-2: 35308580G/T
2-2: 35363539C/T
2-2: 35364644G/T
2-2: 35366081A/G
2-2: 35366090C/T
2-2: 35366129T/C
2-2: 35548874G/A
2-2: 35590458G/A
2-2: 35774598T/C
2-2: 35862833G/A
2-2: 35973747A/C
2-2: 36034249A/G
2-2: 36043614C/T
2-2: 36074145G/A
2-2: 36089488T/A
2-2: 36096834C/T
2-2: 36097555G/C
2-2: 36127510C/G
2-2: 36146345A/C
2-2: 36183579G/C
2-2: 36227735G/A
2-2: 36284698A/G
2-2: 36382147G/T
2-2: 36397389A/G
2-2: 36429639G/A
2-2: 36457235G/A
2-2: 36473373G/A
2-2: 36576925T/C
2-2: 36578342C/T
2-2: 36615530G/A
2-2: 36694475C/T
2-2: 36708301T/G
2-2: 36720857T/C
2-2: 36746527A/T
2-2: 36780966C/T
2-2: 36792299T/C
2-2: 36988360C/T
2-2: 36989424A/G
2-2: 37007305G/A
2-2: 37010647C/T
2-2: 37017461T/C
2-2: 37039495A/C
2-2: 37062432T/C
2-2: 37063978G/T
2-2: 37097455G/A
2-2: 37406760G/C
2-2: 37440223G/A
2-2: 37442544G/A
2-2: 37442587T/C
2-2: 37442653A/G
2-2: 37455283C/A
2-2: 37474449G/T
2-2: 37525651A/G
2-2: 37526121C/A
2-2: 37603596T/G
2-2: 37634448C/T
2-2: 37722152G/A
2-2: 37791426G/T
2-2: 37834885C/T
2-2: 37920911C/T
2-2: 37973459T/C
2-2: 38000921T/C
2-2: 38024226G/T
2-2: 38049531A/G
2-2: 38149475C/T
2-2: 38172204G/A
2-2: 38314318A/T
2-2: 38426143A/C
2-2: 38512101A/C
2-2: 38603273C/A
2-2: 38659873T/G
2-2: 38721855T/C
2-2: 38752239G/A
2-2: 38863599A/G
2-2: 38906789G/T
2-2: 39015028A/T
2-2: 39026995C/T
2-2: 39102685C/T
2-2: 39155490A/T
2-2: 39161094G/T
2-2: 39171600G/C
2-2: 39204483G/A
2-2: 39326421T/C
2-2: 39397392C/T
2-2: 39579942C/A
2-2: 39628456A/G
2-2: 39705161G/A
2-2: 39721762A/C
2-2: 39744572G/A
2-2: 39816243G/T
2-2: 39871432G/A
2-2: 39956533C/A
2-2: 39984029G/A
2-2: 40021704T/A
2-2: 40069297T/A
2-2: 40104711A/G
2-2: 40127896A/C
2-2: 40146197T/G
2-2: 40146649T/C
2-2: 40149028T/C
2-2: 40180732C/T
2-2: 40264965T/G
2-2: 40305129G/C
2-2: 40344513T/C
2-2: 40376072T/C
2-2: 40438582C/T
2-2: 40454761A/G
2-2: 40485751G/A
2-2: 40512689G/A
2-2: 40549437A/T
2-2: 40666536T/C
2-2: 40712858C/T
2-2: 40750250C/T
2-2: 40867886C/T
2-2: 40931171G/A
2-2: 40941748C/A
2-2: 40959282T/C
2-2: 40986419T/G
2-2: 41062025T/C
2-2: 41133611G/A
2-2: 41164545G/A
2-2: 41228889C/A
2-2: 41315815T/C
2-2: 41378999T/A
2-2: 41414116C/T
2-2: 41429339G/A
2-2: 41440186C/A
2-2: 41467180T/A
2-2: 41469425G/T
2-2: 41565262G/T
2-2: 41683843A/G
2-2: 41695708G/A
2-2: 41718178C/G
2-2: 41761031G/T
2-2: 41844999C/T
2-2: 41845189G/.
2-2: 41856855C/T
2-2: 41899398C/T
2-2: 41959593T/G
2-2: 41992665C/T
2-2: 42038714T/C
2-2: 42057753G/A
2-2: 42096456G/T
2-2: 42103664C/A
2-2: 42103668T/A
2-2: 42360747A/G
2-2: 42479687T/C
2-2: 42551288C/A
2-2: 42581850G/A
2-2: 42637303G/A
2-2: 42746201G/A
2-2: 42763999A/G
2-2: 42780084T/C
2-2: 42787384C/T
2-2: 42823713C/G
2-2: 42840341G/T
2-2: 42874784A/G
2-2: 42938401G/C
2-2: 43019071G/C
2-2: 43070705C/T
2-2: 43152423C/A
2-2: 43174515A/G
2-2: 43174717G/A
2-2: 43174718C/A
2-2: 43178374C/T
2-2: 43312702A/T
2-2: 43354588A/T
2-2: 43409450C/T
2-2: 43476408T/G
2-2: 43551165C/A
2-2: 43644548T/A
2-2: 43687026G/T
2-2: 43730774T/C
2-2: 43811246A/C
2-2: 43851361A/C
2-2: 43872712G/A
2-2: 43886267T/A
2-2: 43887902C/T
2-2: 43918225G/A
2-2: 43995236A/G
2-2: 44050633T/A
2-2: 44082108C/T
2-2: 44205445G/C
2-2: 44219251G/A
2-2: 44329183T/C
2-2: 44405616A/G
2-2: 44412371T/C
2-2: 44439044G/T
2-2: 44478139A/C
2-2: 44607582C/T
2-2: 44655548A/T
2-2: 44770676A/G
2-2: 44869176T/C
2-2: 44948737A/G
2-2: 44994727A/T
2-2: 45070831G/A
2-2: 45198611A/T
2-2: 45283411G/A
2-2: 45341235T/A
2-2: 45344436A/G
2-2: 45344438A/T
2-2: 45344458A/T
2-2: 45344491T/A
2-2: 45344512A/C
2-2: 45547730G/A
2-2: 45549570T/A
2-2: 45557908G/C
2-2: 45597186T/A
2-2: 45609112A/G
2-2: 45659979G/A
2-2: 45688022G/A
2-2: 45693736C/A
2-2: 45693957T/G
2-2: 45703670A/T
2-2: 45717674A/G
2-2: 45728809T/C
2-2: 45750512G/A
2-2: 45763366T/C
2-2: 45765197A/G
2-2: 45765198T/A
2-2: 45765573T/A
2-2: 45765693A/C
2-2: 45766265A/G
2-2: 45771186G/A
2-2: 45775113C/T
2-2: 45777352G/A
2-2: 45777540T/A
2-2: 45777544G/A
2-2: 45777726G/A
2-2: 45777879C/A
2-2: 45796291C/T
2-2: 45935720C/T
2-2: 45937690C/A
2-2: 45938060C/T
2-2: 45938215C/T
2-2: 46005457A/G
2-2: 46018352T/A
2-2: 46036235T/A
2-2: 46042430G/A
2-2: 46049045C/T
2-2: 46127179G/A
2-2: 46132945G/T
2-2: 46150434G/A
2-2: 46195353T/C
2-2: 46255887G/T
2-2: 46345586G/A
2-2: 46372692C/G
2-2: 46384679T/C
2-2: 46491522G/T
2-2: 46541869T/C
2-2: 46632915T/G
2-2: 46659958A/T
2-2: 46690894G/T
2-2: 46718599A/G
2-2: 46774630A/T
2-2: 46786109T/C
2-2: 46852294C/A
2-2: 46858374A/T
2-2: 46858777T/A
2-2: 46878594C/T
2-2: 46976860A/G
2-2: 46991884C/T
2-2: 47019812G/A
2-2: 47086652A/T
2-2: 47092118G/A
2-2: 47094043A/G
2-2: 47116438G/A
2-2: 47143433A/G
2-2: 47213857T/C
2-2: 47249267T/C
2-2: 47282553T/A
2-2: 47290834A/G
2-2: 47297131C/T
2-2: 47297806A/G
2-2: 47314441A/G
2-2: 47333010G/A
2-2: 47355159T/C
2-2: 47380690G/C
2-2: 47389079A/G
2-2: 47410603C/T
2-2: 47415625G/T
2-2: 47446991T/C
2-2: 47530145A/G
2-2: 47571647T/A
2-2: 47629952C/T
2-2: 47667427A/T
2-2: 47678702T/C
2-2: 47746571T/C
2-2: 47794557A/G
2-2: 47882149C/T
2-2: 47958563T/C
2-2: 48042922T/C
2-2: 48077463T/A
2-2: 48112385T/C
2-2: 48154590A/C
2-2: 48182839T/C
2-2: 48219221T/A
2-2: 48252012C/T
2-2: 48308477G/A
2-2: 48358957G/A
2-2: 48391512A/T
2-2: 48419392A/G
2-2: 48445293G/A
2-2: 48479912T/C
2-2: 48492621G/A
2-2: 48589379C/A
2-2: 48593160A/T
2-2: 48625246A/G
2-2: 48668106A/G
2-2: 48715859C/G
2-2: 48730119G/T
2-2: 48732325C/A
2-2: 48771586T/G
2-2: 48786218G/A
2-2: 48800050C/T
2-2: 48837063A/T
2-2: 48859592A/C
2-2: 48898201G/C
2-2: 49036668T/C
2-2: 49058989T/C
2-2: 49139049G/A
2-2: 49177156A/C
2-2: 49214127C/T
2-2: 49269621T/C
2-2: 49384402A/C
2-2: 49454696G/T
2-2: 49530044G/A
2-2: 49544617G/T
2-2: 49554700A/C
2-2: 49605176G/T
2-2: 49683505A/G
2-2: 49694228A/G
2-2: 49781914A/T
2-2: 49823691G/C
2-2: 49824253G/A
2-2: 49830819C/A
2-2: 49920931C/T
2-2: 49949406T/C
2-2: 49970682T/G
2-2: 49993162T/C
2-2: 50020373G/T
2-2: 50034434A/T
2-2: 50050612G/A
2-2: 50069181G/C
2-2: 50116860T/G
2-2: 50174567C/T
2-2: 50328455C/T
2-2: 50418502G/A
2-2: 50503585A/G
2-2: 50548540C/T
2-2: 50591780G/A
2-2: 50671234A/G
2-2: 50757089A/T
2-2: 50769035T/C
2-2: 50794181C/T
2-2: 50828499T/C
2-2: 50857492G/A
2-2: 50878770A/G
2-2: 50880090T/A
2-2: 50892304T/C
2-2: 50932416G/A
2-2: 50958128T/C
2-2: 50969956G/T
2-2: 51061343G/A
2-2: 51076794C/T
2-2: 51087670G/T
2-2: 51123573A/G
2-2: 51166052T/A
2-2: 51179473A/G
2-2: 51190898A/T
2-2: 51258913G/C
2-2: 51304386T/G
2-2: 51371311A/G
2-2: 51404551T/C
2-2: 51435679G/A
2-2: 51494880T/C
2-2: 51612772T/C
2-2: 51654798C/A
2-2: 51656177G/C
2-2: 51795286T/C
2-2: 51847438G/A
2-2: 51863654G/A
2-2: 51874944T/C
2-2: 51878389T/C
2-2: 51890356A/G
2-2: 51955354G/A
2-2: 52013036A/C
2-2: 52074774T/C
2-2: 52217806T/C
2-2: 52352889G/C
2-2: 52385235G/A
2-2: 52385250A/G
2-2: 52385251A/T
2-2: 52569884A/G
2-2: 52608771T/A
2-2: 52626987C/G
2-2: 52658480G/A
2-2: 52675793G/A
2-2: 52697603A/T
2-2: 52740123C/G
2-2: 52775350A/G
2-2: 52794386G/A
2-2: 52814223G/T
2-2: 52830363G/T
2-2: 52861014T/C
2-2: 52920680G/A
2-2: 52974314T/A
2-2: 53037764A/G
2-2: 53083885A/C
2-2: 53163460G/A
2-2: 53193141G/C
2-2: 53193262C/G
2-2: 53217386A/G
2-2: 53340837C/T
2-2: 53426247G/T
2-2: 53428664C/T
2-2: 53553610A/C
2-2: 53618364T/C
2-2: 53729271T/C
2-2: 53730773T/G
2-2: 53745630A/C
2-2: 53761501C/T
2-2: 53766252T/A
2-2: 53781967C/G
2-2: 53810504T/C
2-2: 53826514G/A
2-2: 53826573T/C
2-2: 53876030C/T
2-2: 53902979A/G
2-2: 53914635A/G
2-2: 53931691G/A
2-2: 53932825A/T
2-2: 53963755T/C
2-2: 53964736A/G
2-2: 53966048G/A
2-2: 54004889T/C
2-2: 54052936G/C
2-2: 54139014G/A
2-2: 54195248T/C
2-2: 54208044T/C
2-2: 54268750G/A
2-2: 54332620A/G
2-2: 54354543C/A
2-2: 54378444G/T
2-2: 54400284T/C
2-2: 54424161A/G
2-2: 54474872G/A
2-2: 54508261T/G
2-2: 54553764T/C
2-2: 54586215G/A
2-2: 54648189A/G
2-2: 54667927C/A
2-2: 54668962A/G
2-2: 54690447C/T
2-2: 54716343G/A
2-2: 54768345C/T
2-2: 54837355G/T
2-2: 54915247G/A
2-2: 55007675G/A
2-2: 55039924G/A
2-2: 55050649C/T
2-2: 55208914G/A
2-2: 55333219A/G
2-2: 55364830G/T
2-2: 55403149C/T
2-2: 55414190T/C
2-2: 55421230T/G
2-2: 55426069C/T
2-2: 55615462C/T
2-2: 55642690A/C
2-2: 55661668T/A
2-2: 55749704G/A
2-2: 55894680G/A
2-2: 55896523T/C
2-2: 55958941C/A
2-2: 55961131T/A
2-2: 55961139A/G
2-2: 55962711G/A
2-2: 55989436C/T
2-2: 56011969T/C
2-2: 56031730C/T
2-2: 56223584A/G
2-2: 56233745T/C
2-2: 56305855A/C
2-2: 56316884A/G
2-2: 56339879T/A
2-2: 56381454C/T
2-2: 56540490G/A
2-2: 56559103T/C
2-2: 56563463A/G
2-2: 56645476G/C
2-2: 56656771T/C
2-2: 56701586A/G
2-2: 56738304G/C
2-2: 56890137C/A
2-2: 56921575T/C
2-2: 56952109A/G
2-2: 57039637C/T
2-2: 57081430T/C
2-2: 57127839C/T
2-2: 57201525T/A
2-2: 57253133G/T
2-2: 57254263A/G
2-2: 57267273G/A
2-2: 57371641A/G
2-2: 57590288T/G
2-2: 57691261G/A
2-2: 57842865C/T
2-2: 57846996C/T
2-2: 57878798T/A
2-2: 57903040A/G
2-2: 57903368C/A
2-2: 57939511A/C
2-2: 58005689T/A
2-2: 58048262T/G
2-2: 58091948G/A
2-2: 58240551A/C
2-2: 58453338A/G
2-2: 58622125T/C
2-2: 58959342G/A
2-2: 58960135G/A
2-2: 58960251T/C
2-2: 58965917G/A
2-2: 58978030G/A
2-2: 59056613T/G
2-2: 59074029T/C
2-2: 59173318C/T
2-2: 59193079A/G
2-2: 59263115G/A
2-2: 59315032A/G
2-2: 59425746G/A
2-2: 59455061A/T
2-2: 59475194G/A
2-2: 59552689C/G
2-2: 59567511A/T
2-2: 59578134C/T
2-2: 59620739C/A
2-2: 59625339G/T
2-2: 59625397C/T
2-2: 59625653G/T
2-2: 59625663C/T
2-2: 59647095A/G
2-2: 59698579C/T
2-2: 59708685T/C
2-2: 59777390G/A
2-2: 59849725G/A
2-2: 59922602T/G
2-2: 59964077G/A
2-2: 59986543T/C
2-2: 60004925C/T
2-2: 60060866C/T
2-2: 60089925G/A
2-2: 60143468A/G
2-2: 60154974C/T
2-2: 60197212T/C
2-2: 60238204T/C
2-2: 60265041T/A
2-2: 60317763G/A
2-2: 60361158T/C
2-2: 60456051T/A
2-2: 60467102C/T
2-2: 60474596C/T
2-2: 60475084C/T
2-2: 60477523G/T
2-2: 60513416A/G
2-2: 60577215C/T
2-2: 60641226G/A
2-2: 60728531A/T
2-2: 60758583T/C
2-2: 60759807T/G
2-2: 60792493A/C
2-2: 60847743C/T
2-2: 60911382G/A
2-2: 60963188T/C
2-2: 60965609C/T
2-2: 61136651G/A
2-2: 61157132G/T
2-2: 61166792T/A
2-2: 61182619C/G
2-2: 61224102C/T
2-2: 61295488T/A
2-2: 61381032C/A
2-2: 61432320T/C
2-2: 61448392C/T
2-2: 61496171G/A
2-2: 61640962T/C
2-2: 61721181T/A
2-2: 61722983T/A
2-2: 61734026G/A
2-2: 61778746G/T
2-2: 61853462C/T
2-2: 61904198G/A
2-2: 61958173A/G
2-2: 61960167T/G
2-2: 62046732T/C
2-2: 62046740C/T
2-2: 62114592G/C
2-2: 62211541A/T
2-2: 62244048C/G
2-2: 62340045A/T
2-2: 62449625G/A
2-2: 62477071C/T
2-2: 62498336C/T
2-2: 62514842A/G
2-2: 62540601G/T
2-2: 62580936T/A
2-2: 62657757T/G
2-2: 62659246A/G
2-2: 62663220T/A
2-2: 62668197G/A
2-2: 62679635T/A
2-2: 62735275A/G
2-2: 62736533T/C
2-2: 62756879T/G
2-2: 62848710G/A
2-2: 62917924C/T
2-2: 62977024G/T
2-2: 62986855A/G
2-2: 63025469A/G
2-2: 63044492C/T
2-2: 63055755G/A
2-2: 63079487A/G
2-2: 63092996A/C
2-2: 63121081G/A
2-2: 63147934C/T
2-2: 63247324G/A
2-2: 63434404T/C
2-2: 63494953C/A
2-2: 63513735C/T
2-2: 63551817A/G
2-2: 63601854T/G
2-2: 63643996T/C
2-2: 63700083A/C
2-2: 63700229G/T
2-2: 63701703T/G
2-2: 63743589G/A
2-2: 63746746A/G
2-2: 63801644G/A
2-2: 63930217G/A
2-2: 63971291C/A
2-2: 63992641C/T
2-2: 64073417C/T
2-2: 64209576G/C
2-2: 64298517T/A
2-2: 64448985A/G
2-2: 64579563C/T
2-2: 64743583G/A
2-2: 64810206C/T
2-2: 64820742G/A
2-2: 64868389T/C
2-2: 64879252G/A
2-2: 64939936C/T
2-2: 65063074C/A
2-2: 65166526C/A
2-2: 65196310C/T
2-2: 65219483A/G
2-2: 65241185A/G
2-2: 65261364A/T
2-2: 65333677A/G
2-2: 65351107T/C
2-2: 65379770C/A
2-2: 65440806A/G
2-2: 65563280C/T
2-2: 65679195T/A
2-2: 65705696A/G
2-2: 65717723T/C
2-2: 65863565A/G
2-2: 65872269G/A
2-2: 65996472A/G
2-2: 66011570T/A
2-2: 66044052G/C
2-2: 66157112G/A
2-2: 66159459T/A
2-2: 66363215A/G
2-2: 66484782G/T
2-2: 66521163C/T
2-2: 66575945A/G
2-2: 66708269A/G
2-2: 66755525A/C
2-2: 66785854T/C
2-2: 66852071G/C
2-2: 66970965T/G
2-2: 66982491A/T
2-2: 67049055T/C
2-2: 67164278C/T
2-2: 67179213T/A
2-2: 67181552T/A
2-2: 67675200C/A
2-2: 67675314A/C
2-2: 67678415G/T
2-2: 67685389A/T
2-2: 67736747G/A
2-2: 67771632G/T
2-2: 67775621C/T
2-2: 67775798C/T
2-2: 67788047A/C
2-2: 67817714A/G
2-2: 67861103T/A
2-2: 67884416C/T
2-2: 68485920A/G
2-2: 68498344T/C
2-2: 68516050G/A
2-2: 68544032G/A
2-2: 68627124C/T
2-2: 68629149A/T
2-2: 68655996T/C
2-2: 68720721G/A
2-2: 68738990G/A
2-2: 68745833T/A
2-2: 68771130T/C
2-2: 68858479C/T
2-2: 68956046C/A
2-2: 69049192T/G
2-2: 69077026G/C
2-2: 69093414A/T
2-2: 69106922A/T
2-2: 69186306T/A
2-2: 69315315T/C
2-2: 69397849T/C
2-2: 69425178A/G
2-2: 69432715C/G
2-2: 69436137C/T
2-2: 69448433A/G
2-2: 69545966G/T
2-2: 69779254G/T
2-2: 69785837A/C
2-2: 69805826C/T
2-2: 70016779A/G
2-2: 70108601C/A
2-2: 70593359A/T
2-2: 70705193C/T
2-3: 16556A/C
2-3: 286191A/G
2-3: 567170A/G
2-3: 694234C/T
2-3: 788199T/A
2-3: 828712T/A
2-3: 1053050A/G
2-3: 1115581C/A
2-3: 1137384C/T
2-3: 1150992A/G
2-3: 1165321C/T
2-3: 1266058C/G
2-3: 1329905G/T
2-3: 1432832T/A
2-3: 1635809A/T
2-3: 1697689T/C
2-3: 1763092G/C
2-3: 1782165A/G
2-3: 1796533A/T
2-3: 1817736T/C
2-3: 2001251T/C
2-3: 2024264T/C
2-3: 2094048T/C
2-3: 2161911T/C
2-3: 2163021G/A
2-3: 2173352C/T
2-3: 2186528T/C
2-3: 2302763G/C
2-3: 2324700G/T
2-3: 2458607A/G
2-3: 2460339A/G
2-3: 2460626G/T
2-3: 2460846T/C
2-3: 2596953G/A
2-3: 2600614G/T
2-3: 2734546G/C
2-3: 2763736T/C
2-3: 2797830G/A
2-3: 2904538T/A
2-3: 2926264T/A
2-3: 2939626G/A
2-3: 2993061T/A
2-3: 3029419G/A
2-3: 3123958A/G
2-3: 3183205C/T
2-3: 3244622C/T
2-3: 3283908C/T
2-3: 3289034G/A
2-3: 3294272T/C
2-3: 3340034G/T
2-3: 3463784A/G
2-3: 3478462C/T
2-3: 3493721C/T
2-3: 3536130A/G
2-3: 3566663T/C
2-3: 3649551G/A
2-3: 3650257G/A
2-3: 3650815A/C
2-3: 3694646T/A
2-3: 3727070G/A
2-3: 3753775A/C
2-3: 3774535A/G
2-3: 3917689G/T
2-3: 3981604C/T
2-3: 4116347A/C
2-3: 4233554G/A
2-3: 4290764T/C
2-3: 4319772T/C
2-3: 4352262G/A
2-3: 4382658A/T
2-3: 4470414A/G
2-3: 4497109T/C
2-3: 4503486G/A
2-3: 4601986A/T
2-3: 4657490T/C
2-3: 4769099A/T
2-3: 4809945C/G
2-3: 4898609T/A
2-3: 4954419T/G
2-3: 5147145T/C
2-3: 5160148G/C
2-3: 5189299C/T
2-3: 5275158A/G
2-3: 5302354C/T
2-3: 5302887C/T
2-3: 5335117C/T
2-3: 5380044A/G
2-3: 5417698G/A
2-3: 5455118C/A
2-3: 5458998A/G
2-3: 5473460C/A
2-3: 5585914G/A
2-3: 5672716T/C
2-3: 5715131A/C
2-3: 5741651A/T
2-3: 5789942C/A
2-3: 5828229T/A
2-3: 6099553A/G
2-3: 6159354A/C
2-3: 6203085A/G
2-3: 6248481A/T
2-3: 6329116G/A
2-3: 6357992C/T
2-3: 6423602C/T
2-3: 6501063A/G
2-3: 6629384A/T
2-3: 6819604A/G
2-3: 6846131T/A
2-3: 6985448C/T
2-3: 7090773G/C
2-3: 7111328C/A
2-3: 7111624T/C
2-3: 7111627A/T
2-3: 7142948A/G
2-3: 7171087T/C
2-3: 7251679G/A
2-3: 7325108T/C
2-3: 7336581A/G
2-3: 7468208G/T
2-3: 7479413C/.
2-3: 7507639G/A
2-3: 7545623T/C
2-3: 7622600C/T
2-3: 7650575A/G
2-3: 7737344T/C
2-3: 7750040A/C
2-3: 7761067C/T
2-3: 7787091A/C
2-3: 7821167G/A
2-3: 7893939G/A
2-3: 7980650T/C
2-3: 8008465G/A
2-3: 8048399G/C
2-3: 8077317A/T
2-3: 8166668T/C
2-3: 8180055C/T
2-3: 8201269A/G
2-3: 8230134A/G
2-3: 8244310A/T
2-3: 8245378T/G
2-3: 8246070C/A
2-3: 8276664A/G
2-3: 8305192A/T
2-3: 8359148C/T
2-3: 8385903C/T
2-3: 8429689T/A
2-3: 8536755T/G
2-3: 8623137A/G
2-3: 8687128T/C
2-3: 8713340T/C
2-3: 8797755G/T
2-3: 8827017A/C
2-3: 8863606T/A
2-3: 8891272C/T
2-3: 8891459G/A
2-3: 8895074G/A
2-3: 8937314A/C
2-3: 8937687T/A
2-3: 8938473G/A
2-3: 8938617G/T
2-3: 8938711G/A
2-3: 8938814C/G
2-3: 8938862C/T
2-3: 8938938T/G
2-3: 8939148T/C
2-3: 8939335A/C
2-3: 8939365C/A
2-3: 8939547A/G
2-3: 8939715C/T
2-3: 8939756C/G
2-3: 8939880G/C
2-3: 8940029T/C
2-3: 8940238C/G
2-3: 8940385C/T
2-3: 8940443G/A
2-3: 8940546T/G
2-3: 8940653A/G
2-3: 8940890C/T
2-3: 8940902A/G
2-3: 8940904A/G
2-3: 8943727C/T
2-3: 8943779G/A
2-3: 8943834T/C
2-3: 8944911A/G
2-3: 8945334A/G
2-3: 8945366C/A
2-3: 8945475G/A
2-3: 8945689C/T
2-3: 8945938G/A
2-3: 8947778C/T
2-3: 8951854T/C
2-3: 8952158T/A
2-3: 8998298C/T
2-3: 9183261T/A
2-3: 9232818T/G
2-3: 9285000T/C
2-3: 9297873C/G
2-3: 9336529A/C
2-3: 9390701A/G
2-3: 9436904A/G
2-3: 9475609G/A
2-3: 9491744A/G
2-3: 9612152T/C
2-3: 9619910T/G
2-3: 9724630G/T
2-3: 9766856T/C
2-3: 9832883C/T
2-3: 9867289T/C
2-3: 9919672T/C
2-3: 9965153G/A
2-3: 10081134T/G
2-3: 10098370T/A
2-3: 10186380T/A
2-3: 10234014A/G
2-3: 10262059C/A
2-3: 10322400C/T
2-3: 10460178A/G
2-3: 10481520C/T
2-3: 10510118G/T
2-3: 10595389G/A
2-3: 10646719T/A
2-3: 10712330G/A
2-3: 10752082G/C
2-3: 10787269G/A
2-3: 10871647T/G
2-3: 10958188T/C
2-3: 10969637A/G
2-3: 11002616A/G
2-3: 11052072C/G
2-3: 11118796G/C
2-3: 11174016T/G
2-3: 11204466G/A
2-3: 11547656A/G
2-3: 11559796G/A
2-3: 11664927C/T
2-3: 11688158T/C
2-3: 11716139G/A
2-3: 11769637G/A
2-3: 11817423C/T
2-3: 11862878T/C
2-3: 11894525C/T
2-3: 11986761G/T
2-3: 12064516C/A
2-3: 12097699A/T
2-3: 12099077C/T
2-3: 12111629C/A
2-3: 12111650A/G
2-3: 12113749A/C
2-3: 12130568A/C
2-3: 12330212T/A
2-3: 12390725T/A
2-3: 12579170C/T
2-3: 12658586G/C
2-3: 12740511T/C
2-3: 12769679T/C
2-3: 12900476G/A
2-3: 13080088G/T
2-3: 13095749G/A
2-3: 13100481C/T
2-3: 13132384G/A
2-3: 13174538T/A
2-3: 13188557A/T
2-3: 13188559T/C
2-3: 13188602T/C
2-3: 13188614C/T
2-3: 13189194T/C
2-3: 13189200G/.
2-3: 13212814C/T
2-3: 13260925C/G
2-3: 13324542C/T
2-3: 13396506T/G
2-3: 13422732T/C
2-3: 13597943A/G
2-3: 13649906G/A
2-3: 13688191T/A
2-3: 13718073C/T
2-3: 13757145G/T
2-3: 13819859T/A
2-3: 13839863G/C
2-3: 13922906T/C
2-3: 14070874G/A
2-3: 14097726C/T
2-3: 14123628T/C
2-3: 14138950C/A
2-3: 14218739G/A
2-3: 14249538A/G
2-3: 14272242G/T
2-3: 14308331G/T
2-3: 14319124A/T
2-3: 14418658T/C
2-3: 14464280A/C
2-3: 14502483C/T
2-3: 14561394C/T
2-3: 14580640A/T
2-3: 14602589C/T
2-3: 14613863T/C
2-3: 14656016C/T
2-3: 14659653C/A
2-3: 14661704T/A
2-3: 14665491T/C
2-3: 14665494C/T
2-3: 14668121C/G
2-3: 14668430A/T
2-3: 14675621T/G
2-3: 14679029G/T
2-3: 14683229G/A
2-3: 14776147A/C
2-3: 14912955T/A
2-3: 15055544T/C
2-3: 15086490G/A
2-3: 15087071T/A
2-3: 15112122C/T
2-3: 15132401T/A
2-3: 15147026T/C
2-3: 15163010C/T
2-3: 15195467A/G
2-3: 15218650T/A
2-3: 15260936A/G
2-3: 15355071A/G
2-3: 15380211G/T
2-3: 15456415A/T
2-3: 15490611A/C
2-3: 15490612T/A
2-3: 15506073G/A
2-3: 15558296C/T
2-3: 15600020G/A
2-3: 15639608C/A
2-3: 15724202A/G
2-3: 15749295G/A
2-3: 15818075C/A
2-3: 15878556T/C
2-3: 16032577A/T
2-3: 16111844C/T
2-3: 16168217A/G
2-3: 16254505A/G
2-3: 16257581A/G
2-3: 16257721T/A
2-3: 16389081G/A
2-3: 16423352G/A
2-3: 16424770A/C
2-3: 16435010G/A
2-3: 16435922T/C
2-3: 16436419A/T
2-3: 16436477G/A
2-3: 16448404T/C
2-3: 16455673A/T
2-3: 16460291T/A
2-3: 16514035G/A
2-3: 16571624C/T
2-3: 16662900G/A
2-3: 16710484T/G
2-3: 16888461T/C
2-3: 17001060T/A
2-3: 17073308A/C
2-3: 17104660T/A
2-3: 17206980A/T
2-3: 17246856A/T
2-3: 17259472T/C
2-3: 17282896T/C
2-3: 17353046G/A
2-3: 17353277G/A
2-3: 17353439A/C
2-3: 17353585T/C
2-3: 17364746C/T
2-3: 17402980T/C
2-3: 17426593C/T
2-3: 17526242G/T
2-3: 17602422T/C
2-3: 17680299A/G
2-3: 17701120T/C
2-3: 17788953G/A
2-3: 17812204G/A
2-3: 17909686T/A
2-3: 17922481G/A
2-3: 17989523G/C
2-3: 18039970A/T
2-3: 18058611A/G
2-3: 18230880A/T
2-3: 18241228A/G
2-3: 18288061T/G
2-3: 18387779C/T
2-3: 18392550T/A
2-3: 18411546G/A
2-3: 18421577C/T
2-3: 18486145A/G
2-3: 18490470C/G
2-3: 18490749C/T
2-3: 18491105G/A
2-3: 18491187C/T
2-3: 18493217A/T
2-3: 18494488A/G
2-3: 18494592C/A
2-3: 18494721C/T
2-3: 18502942T/G
2-3: 18517566T/C
2-3: 18561969C/A
2-3: 18574543T/G
2-3: 18622547A/T
2-3: 18627792G/A
2-3: 18641397T/A
2-3: 18646358A/G
2-3: 18649994A/T
2-3: 18651142T/A
2-3: 18651502C/T
2-3: 18654736G/A
2-3: 18775910T/A
2-3: 18805247T/A
2-3: 18857450T/A
2-3: 18896475A/G
2-3: 18949648T/C
2-3: 19050821G/A
2-3: 19050924T/C
2-3: 19075419C/T
2-3: 19076813A/C
2-3: 19087338C/T
2-3: 19096219A/C
2-3: 19097466C/T
2-3: 19126145A/T
2-3: 19159424A/G
2-3: 19225763A/T
2-3: 19229624C/T
2-3: 19242004T/C
2-3: 19281970C/T
2-3: 19286603A/G
2-3: 19287579T/G
2-3: 19287724A/T
2-3: 19287976C/T
2-3: 19293800G/A
2-3: 19377593C/T
2-3: 19597624G/T
2-3: 19672223C/T
2-3: 19714781T/A
2-3: 19772054G/C
2-3: 19818203T/C
2-3: 19851826C/T
2-3: 20038048C/A
2-3: 20040214T/C
2-3: 20133772G/A
2-3: 20162146C/T
2-3: 20184840A/G
2-3: 20196556T/C
2-3: 20209080C/A
2-3: 20235827T/A
2-3: 20245530T/A
2-3: 20246091G/A
2-3: 20247546A/G
2-3: 20247709A/C
2-3: 20260731C/T
2-3: 20284344G/A
2-3: 20318766C/T
2-3: 20376232G/A
2-3: 20394185T/A
2-3: 20426899G/A
2-3: 20516118T/A
2-3: 20551761A/G
2-3: 20586471T/C
2-3: 20604878T/C
2-3: 20819613A/G
2-3: 20893444G/T
2-3: 20971543A/T
2-3: 20998933G/A
2-3: 21105816A/T
2-3: 21138912G/C
2-3: 21167900A/G
2-3: 21208404G/A
2-3: 21287371G/A
2-3: 21324084T/C
2-3: 21387709A/G
2-3: 21548499A/G
2-3: 21716187T/A
2-3: 21728401T/C
2-3: 21791813G/A
2-3: 21817058A/C
2-3: 21851505C/G
2-3: 21895356C/A
2-3: 21975036T/C
2-3: 22004299A/G
2-3: 22020349T/C
2-3: 22093816C/T
2-3: 22131414T/C
2-3: 22225954T/A
2-3: 22337585C/T
2-3: 22455309T/C
2-3: 22546949T/C
2-3: 22589220G/A
2-3: 22614684T/G
2-3: 22702877A/G
2-3: 22743411G/A
2-3: 22783239C/T
2-3: 22835397C/T
2-3: 22892906C/T
2-3: 22935075C/G
2-3: 22976795C/A
2-3: 23010742G/A
2-3: 23088871A/G
2-3: 23138598T/C
2-3: 23201245C/T
2-3: 23308812T/C
2-3: 23317957C/T
2-3: 23349480A/C
2-3: 23429230G/A
2-3: 23460995G/A
2-3: 23475448T/A
2-3: 23482042A/T
2-3: 23505086C/T
2-3: 23506192C/T
2-3: 23542377C/T
2-3: 23583961A/T
2-3: 23782058G/T
2-3: 23814284T/C
2-3: 23852969T/C
2-3: 23865393T/C
2-3: 23983165A/G
2-3: 24002177G/T
2-3: 24041264T/C
2-3: 24048383G/A
2-3: 24063634C/T
2-3: 24172830A/T
2-3: 24247062C/T
2-3: 24338409A/C
2-3: 24349585T/C
2-3: 24383833G/A
2-3: 24416529C/T
2-3: 24434416G/T
2-3: 24462753A/G
2-3: 24548490T/C
2-3: 24558532T/C
2-3: 24663894G/A
2-3: 24744899G/A
2-3: 24804121T/A
2-3: 24846820G/C
2-3: 24851487G/C
2-3: 24851584C/T
2-3: 24852042C/A
2-3: 24852941T/C
2-3: 24853388T/A
2-3: 24853391G/A
2-3: 24854877A/G
2-3: 24855137A/T
2-3: 24855160T/C
2-3: 24871989A/T
2-3: 24882227G/A
2-3: 24894573G/A
2-3: 24907763C/T
2-3: 24985181C/T
2-3: 25025795C/A
2-3: 25039849C/A
2-3: 25100681G/T
2-3: 25113625G/A
2-3: 25151580T/C
2-3: 25182998A/T
2-3: 25184727G/A
2-3: 25239550A/G
2-3: 25276526A/C
2-3: 25402819C/A
2-3: 25410666G/A
2-3: 25430563G/A
2-3: 25436837G/C
2-3: 25438783A/G
2-3: 25438832A/T
2-3: 25440981G/A
2-3: 25468168G/A
2-3: 25540444G/A
2-3: 25558289C/T
2-3: 25591148T/C
2-3: 25605613C/A
2-3: 25650388A/T
2-3: 25661050G/A
2-3: 25665016T/C
2-3: 25690171T/A
2-3: 25705746T/A
2-3: 25723829G/A
2-3: 25739600T/A
2-3: 25754893T/A
2-3: 25759375A/C
2-3: 25759460T/C
2-3: 25759588C/T
2-3: 25808815A/G
2-3: 25862115T/A
2-3: 25890839T/A
2-3: 25924569A/C
2-3: 25947891G/A
2-3: 25954813A/C
2-3: 26009284A/G
2-3: 26023918A/G
2-3: 26040017G/A
2-3: 26068605T/C
2-3: 26127056G/A
2-3: 26157585G/C
2-3: 26190518C/T
2-3: 26201742C/T
2-3: 26205536C/G
2-3: 26311087A/G
2-3: 26328914C/T
2-3: 26342879T/G
2-3: 26356379A/T
2-3: 26543134G/C
2-3: 26554516G/A
2-3: 26565062T/G
2-3: 26566257C/G
2-3: 26582063C/T
2-3: 26604038C/T
2-3: 26637816G/A
2-3: 26648268C/T
2-3: 26649759C/T
2-3: 26693650G/C
2-3: 26733365G/A
2-3: 26739922A/T
2-3: 26747564C/T
2-3: 26871255T/C
2-3: 27051496C/A
2-3: 27137873T/C
2-3: 27160106C/T
2-3: 27175882C/T
2-3: 27264864G/T
2-3: 27298070G/C
2-3: 27394093T/C
2-3: 27437405T/G
2-3: 27468337G/A
2-3: 27480708G/A
2-3: 27484181A/C
2-3: 27558376G/A
2-3: 27579795A/T
2-3: 27598569T/C
2-3: 27697218G/A
2-3: 27784190G/C
2-3: 27787151T/C
2-3: 27787170A/G
2-3: 27801783A/G
2-3: 27828814A/T
2-3: 27889812C/A
2-3: 27961010G/A
2-3: 28066430C/T
2-3: 28165213T/G
2-3: 28192040T/A
2-3: 28243526T/C
2-3: 28259939C/T
2-3: 28326394T/A
2-3: 28366412A/G
2-3: 28368660C/T
2-3: 28376724G/C
2-3: 28381137T/C
2-3: 28381323A/G
2-3: 28381820A/T
2-3: 28394767C/T
2-3: 28407707A/T
2-3: 28407828G/A
2-3: 28407981C/A
2-3: 28433204C/T
2-3: 28482766A/T
2-3: 28551605G/T
2-3: 28578621T/C
2-3: 28606594T/C
2-3: 28625730G/T
2-3: 28648300T/C
2-3: 28715915C/A
2-3: 28715946T/C
2-3: 28718444G/T
2-3: 28799110G/C
2-3: 28844910G/T
2-3: 28940579G/C
2-3: 28948352A/C
2-3: 28970048A/G
2-3: 29080438A/G
2-3: 29081533C/A
2-3: 29111125C/G
2-3: 29146054T/C
2-3: 29193988A/G
2-3: 29208163G/A
2-3: 29221627C/T
2-3: 29254704A/C
2-3: 29324389T/G
2-3: 29339057A/T
2-3: 29343983T/C
2-3: 29414555T/C
2-3: 29431806T/G
2-3: 29496398T/G
2-3: 29512431G/A
2-3: 29537826C/T
2-3: 29565482G/A
2-3: 29595149G/A
2-3: 29608291G/T
2-3: 29621095T/C
2-3: 29751791A/T
2-3: 29809746A/T
2-3: 29924498T/C
2-3: 30042467T/C
2-3: 30128620T/C
2-3: 30252812T/G
2-3: 30265507G/T
2-3: 30347799G/T
2-3: 30390811T/C
2-3: 30491842A/G
2-3: 30492023T/C
2-3: 30605888A/G
2-3: 30865185G/C
2-3: 30873266G/A
2-3: 30894301C/T
2-3: 31083114A/T
2-3: 31124986G/C
2-3: 31168600T/G
2-3: 31208877G/A
2-3: 31262286G/A
2-3: 31296331T/A
2-3: 31310489T/A
2-3: 31339666G/C
2-3: 31375096C/G
2-3: 31460117C/A
2-3: 31515564C/T
2-3: 31527109G/A
2-3: 31537553C/T
2-3: 31575033C/T
2-3: 31657897C/T
2-3: 31659255T/A
2-3: 31659283G/A
2-3: 31659319A/T
2-3: 31659391C/A
2-3: 31659412G/A
2-3: 31659430A/C
2-3: 31659445A/G
2-3: 31659462T/C
2-3: 31659470G/A
2-3: 31829556A/T
2-3: 31830419T/G
2-3: 31833661T/C
2-3: 31846398T/G
2-3: 31846423G/A
2-3: 31851958G/A
2-3: 31851964T/C
2-3: 31870916T/A
2-3: 31917826A/C
2-3: 31932005A/T
2-3: 31961343G/T
2-3: 31987889C/G
2-3: 32075572G/A
2-3: 32173635A/G
2-3: 32225676G/T
2-3: 32256288T/C
2-3: 32277756G/A
2-3: 32367015C/T
2-3: 32377031A/G
2-3: 32410750A/G
2-3: 32477330T/A
2-3: 32509639G/T
2-3: 32658622G/T
2-3: 32736003C/G
2-3: 32913378A/T
2-3: 32929280G/A
2-3: 32970868A/G
2-3: 33003630C/T
2-3: 33167790T/G
2-3: 33347205A/C
2-3: 33368597A/G
2-3: 33412963G/T
2-3: 33567106G/A
2-3: 33580605T/A
2-3: 33673397G/A
2-3: 33881977C/T
2-3: 33884570G/A
2-3: 33943983G/A
2-3: 33955081T/G
2-3: 34034456A/G
2-3: 34036177T/A
2-3: 34047535C/T
2-3: 34069374A/T
2-3: 34069436A/C
2-3: 34131697T/G
2-3: 34173550G/T
2-3: 34195366C/T
2-3: 34312418C/T
2-3: 34334640A/G
2-3: 34446586T/A
2-3: 34499017A/G
2-3: 34559121C/T
2-3: 34654141T/A
2-3: 34680535C/T
2-3: 34695210A/G
2-3: 34711310G/A
2-3: 34747739A/C
2-3: 34802449T/A
2-3: 34881171C/A
2-3: 34887442G/A
2-3: 34910630C/T
2-3: 34997132A/G
2-3: 35050988C/G
2-3: 35144283C/T
2-3: 35195016C/T
2-3: 35196207T/A
2-3: 35233796G/T
2-3: 35315799A/T
2-3: 35524873C/T
2-3: 35535748T/G
2-3: 35538452C/T
2-3: 35574415T/G
2-3: 35597747A/G
2-3: 35765120C/A
2-3: 35774697C/T
2-3: 35784477C/T
2-3: 35784490T/G
2-3: 35784678C/T
2-3: 35798730T/G
2-3: 35943468G/A
2-3: 35957706T/C
2-3: 35970991A/G
2-3: 36020507G/C
2-3: 36087531G/A
2-3: 36092073C/T
2-3: 36098481A/T
2-3: 36099670C/T
2-3: 36184648T/C
2-3: 36206924C/T
2-3: 36215375G/A
2-3: 36217797C/G
2-3: 36289210G/A
2-3: 36334118C/G
2-3: 36394432G/A
2-3: 36420496T/C
2-3: 36432994T/G
2-3: 36563232G/A
2-3: 36589875T/A
2-3: 36632936T/A
2-3: 36646582T/A
2-3: 36646623A/C
2-3: 36682745A/G
2-3: 36699509T/A
2-3: 36702356C/G
2-3: 36735449T/A
2-3: 36778506A/C
2-3: 36816120C/G
2-3: 36850924T/A
2-3: 36867349G/A
2-3: 36889795A/G
2-3: 36999876C/T
2-3: 37170339C/A
2-3: 37177438A/G
2-3: 37228624T/A
2-3: 37241134T/A
2-3: 37274952T/C
2-3: 37297221C/G
2-3: 37362498G/T
2-3: 37628079T/C
2-3: 37757929G/A
2-3: 37834575T/A
2-3: 37938768C/T
2-3: 37981429G/A
2-3: 38065311T/C
2-3: 38105985C/T
2-3: 38208925G/A
2-3: 38228926T/A
2-3: 38275067T/C
2-3: 38365883T/C
2-3: 38394993C/T
2-3: 38440639T/C
2-3: 38466877G/A
2-3: 38472895A/C
2-3: 38483498T/G
2-3: 38523584C/G
2-3: 38543443C/T
2-3: 38558524G/C
2-3: 38582491A/G
2-3: 38680611C/T
2-3: 38860537A/G
2-3: 38944230A/G
2-3: 39088341C/T
2-3: 39269568A/G
2-3: 39272523T/A
2-3: 39338538C/A
2-3: 39394889G/T
2-3: 39395509T/A
2-3: 39397342C/T
2-3: 39398323A/G
2-3: 39398444A/G
2-3: 39398555C/T
2-3: 39398594A/G
2-3: 39398858A/T
2-3: 39407309T/G
2-3: 39411509A/T
2-3: 39457841A/G
2-3: 39480475G/T
2-3: 39498599G/A
2-3: 39511092T/G
2-3: 39547142A/G
2-3: 39597574T/C
2-3: 39668566C/A
2-3: 39715688C/G
2-3: 39743893G/A
2-3: 39761770A/T
2-3: 39773441G/A
2-3: 39806151T/C
2-3: 39831460C/T
2-3: 39871400C/T
2-3: 39927572A/T
2-3: 39994853A/G
2-3: 40019345T/C
2-3: 40133820C/A
2-3: 40146164A/G
2-3: 40146658C/T
2-3: 40162351A/G
2-3: 40186954T/A
2-3: 40205515T/C
2-3: 40259024A/T
2-3: 40278240T/G
2-3: 40286118C/T
2-3: 40295161C/T
2-3: 40335978T/C
2-3: 40381052C/A
2-3: 40451358T/A
2-3: 40478185C/A
2-3: 40488952T/G
2-3: 40495565C/T
2-3: 40499046T/C
2-3: 40509956A/C
2-3: 40526969A/G
2-3: 40576605A/G
2-3: 40601161G/A
2-3: 40611048G/A
2-3: 40611161G/A
2-3: 40611329C/T
2-3: 40614418C/T
2-3: 40614523T/C
2-3: 40614680T/C
2-3: 40619835G/A
2-3: 40619852C/T
2-3: 40620268G/A
2-3: 40620387A/G
2-3: 40620392G/A
2-3: 40620569G/A
2-3: 40628545T/C
2-3: 40663208A/T
2-3: 40686302A/G
2-3: 40755760T/C
2-3: 40820624T/A
2-3: 40852857T/C
2-3: 40863497A/C
2-3: 40891647C/T
2-3: 40939024A/G
2-3: 40990038A/T
2-3: 41081510C/T
2-3: 41157560T/C
2-3: 41182746G/A
2-3: 41225029G/A
2-3: 41226907T/A
2-3: 41240570T/C
2-3: 41272110C/T
2-3: 41288402A/G
2-3: 41298621C/G
2-3: 41333256C/T
2-3: 41350888A/T
2-3: 41427677A/G
2-3: 41447240A/G
2-3: 41489918A/C
2-3: 41513730A/G
2-3: 41531404T/C
2-3: 41542428C/T
2-3: 41552687T/A
2-3: 41555558T/C
2-3: 41556953G/A
2-3: 41557134G/C
2-3: 41557559A/G
2-3: 41622227C/T
2-3: 41783654A/G
2-3: 41810664T/A
2-3: 41931879T/A
2-3: 41976897G/A
2-3: 42034865C/T
2-3: 42050588T/A
2-3: 42063936C/A
2-3: 42145917A/G
2-3: 42155378C/T
2-3: 42155483A/C
2-3: 42159492C/T
2-3: 42171442A/T
2-3: 42173185G/A
2-3: 42207049A/T
2-3: 42260196T/C
2-3: 42312548G/A
2-3: 42351208T/C
2-3: 42364461C/T
2-3: 42368406T/A
2-3: 42371485A/C
2-3: 42373786C/T
2-3: 42390019C/A
2-3: 42411871T/C
2-3: 42426404T/G
2-3: 42434811T/C
2-3: 42493937G/A
2-3: 42527659C/T
2-3: 42540373T/C
2-3: 42554690G/A
2-3: 42563360C/T
2-3: 42586190C/T
2-3: 42602382G/A
2-3: 42611798C/T
2-3: 42612291C/T
2-3: 42612361G/A
2-3: 42613923C/G
2-3: 42615470T/G
2-3: 42629070C/T
2-3: 42631575G/A
2-3: 42668095C/T
2-3: 42692182A/G
2-3: 42696843A/G
2-3: 42696857C/T
2-3: 42707107A/C
2-3: 42811347T/A
2-3: 42907519T/C
2-3: 42912942G/C
2-3: 42927382A/T
2-3: 42937927C/A
2-3: 42955292G/C
2-3: 42973800T/A
2-3: 42981906C/T
2-3: 42982212T/C
2-3: 42999549T/A
2-3: 43100667A/C
2-3: 43122667A/G
2-3: 43257490A/T
2-3: 43318748G/A
2-3: 43387107A/G
2-3: 43533589G/C
2-3: 43645009C/T
2-3: 43671066T/C
2-3: 43686710C/T
2-3: 43686787G/A
2-3: 43686809G/C
2-3: 43726973G/T
2-3: 43727685C/T
2-3: 43727795C/A
2-3: 43772163T/A
2-3: 43810356T/A
2-3: 43865401C/A
2-3: 43944347A/G
2-3: 43998780A/G
2-3: 43998829T/C
2-3: 43998840G/A
2-3: 44023864T/C
2-3: 44110322G/A
2-3: 44199986T/C
2-3: 44202822T/C
2-3: 44282851C/T
2-3: 44354511G/A
2-3: 44406022G/T
2-3: 44436874G/T
2-3: 44481285T/G
2-3: 44543261C/T
2-3: 44595779A/T
2-3: 44602144A/G
2-3: 44634803T/C
2-3: 44667690C/A
2-3: 44771331T/C
2-3: 44772555T/C
2-3: 44796471T/A
2-3: 44824810T/C
2-3: 44866997C/T
2-3: 44882763G/A
2-3: 44892992T/A
2-3: 44946248C/A
2-3: 44963680G/A
2-3: 44988120C/T
2-3: 45000697A/T
2-3: 45028044T/G
2-3: 45097459G/A
2-3: 45138025A/G
2-3: 45150830C/T
2-3: 45189702G/A
2-3: 45234057T/G
2-3: 45238166A/G
2-3: 45276594A/G
2-3: 45324504C/T
2-3: 45364277C/G
2-3: 45403881C/T
2-3: 45431461A/G
2-3: 45444041G/A
2-3: 45450050G/A
2-3: 45484389C/T
2-3: 45524648A/T
2-3: 45552549A/C
2-3: 45588281A/G
2-3: 45614480C/T
2-3: 45618306G/A
2-3: 45632573C/T
2-3: 45653067G/C
2-3: 45690116G/T
2-3: 45707078A/G
2-3: 45750279T/C
2-3: 45760037C/T
2-3: 45792968T/C
2-3: 45944148G/A
2-3: 45951776G/A
2-3: 45993157A/T
2-3: 46060340C/T
2-3: 46172187A/G
2-3: 46202197C/T
2-3: 46313910G/A
2-3: 46423534A/G
2-3: 46455557G/A
2-3: 46467248G/A
2-3: 46495035C/T
2-3: 46495559G/A
2-3: 46523761A/T
2-3: 46637026C/A
2-3: 46647359A/T
2-3: 46664436G/T
2-3: 46680268G/A
2-3: 46698025C/T
2-3: 46700220C/G
2-3: 46713414A/T
2-3: 46725355C/T
2-3: 46725487T/C
2-3: 46755058C/A
2-3: 46827359C/G
2-3: 46850698T/C
2-3: 46869320G/A
2-3: 46938022G/A
2-3: 47049525A/G
2-3: 47049699G/A
2-3: 47107696G/T
2-3: 47213360G/A
2-3: 47233186C/G
2-3: 47301563A/G
2-3: 47403737T/A
2-3: 47434710T/C
2-3: 47546902G/A
2-3: 47679427C/T
2-3: 47697295T/A
2-3: 47793969T/G
2-3: 47874693A/G
2-3: 48003516C/T
2-3: 48068619G/C
2-3: 48132977T/C
2-3: 48215150T/C
2-3: 48256162T/C
2-3: 48312013A/G
2-3: 48339090G/A
2-3: 48359690A/C
2-3: 48440712A/T
2-3: 48469972C/G
2-3: 48524318A/C
2-3: 48559313G/A
2-3: 48612968T/A
2-3: 48685344C/T
2-3: 48726062C/G
2-3: 48756624G/A
2-3: 48772352C/T
2-3: 48788787A/T
2-3: 48798915A/C
2-3: 48809809G/A
2-3: 48823538T/C
2-3: 48844543T/C
2-3: 48963368G/A
2-3: 48975981A/G
2-3: 48991172A/G
2-3: 49142769G/A
2-3: 49218326G/C
2-3: 49292951A/G
2-3: 49348058A/G
2-3: 49450332G/C
2-3: 49468190G/A
2-3: 49498492C/T
2-3: 49506996A/G
2-3: 49507230T/C
2-3: 49507236T/C
2-3: 49507443T/A
2-3: 49507574A/G
2-3: 49507803C/G
2-3: 49507830C/T
2-3: 49508762G/T
2-3: 49509772C/T
2-3: 49512097A/G
2-3: 49512175G/T
2-3: 49522437C/A
2-3: 49545585A/G
2-3: 49603950T/A
2-3: 49652839A/G
2-3: 49701621G/A
2-3: 49711187C/A
2-3: 49711219T/C
2-3: 49711836T/C
2-3: 49712011A/G
2-3: 49712050A/G
2-3: 49717884G/A
2-3: 49718702T/C
2-3: 49719087T/C
2-3: 49719192C/A
2-3: 49720094C/T
2-3: 49733570C/T
2-3: 49763502C/G
2-3: 49803815G/A
2-3: 49852850A/G
2-3: 49882467G/A
2-3: 49919704A/G
2-3: 49927320T/C
2-3: 49934014A/G
2-3: 49960418T/A
2-3: 50006465G/A
2-3: 50019244C/A
2-3: 50043092T/.
2-3: 50050426T/A
2-3: 50093038A/G
2-3: 50190456A/G
2-3: 50404999T/C
2-3: 50411407A/T
2-3: 50508577A/G
2-3: 50522513C/T
2-3: 50529472T/C
2-3: 50542659A/T
2-3: 50582298G/C
2-3: 50609118T/C
2-3: 50622653G/T
2-3: 50749070C/T
2-3: 50812066A/G
2-3: 50838132C/A
2-3: 50860684C/T
2-3: 50864768G/A
2-3: 50885601A/G
2-3: 50898179C/T
2-3: 50902890C/A
2-3: 50910241T/C
2-3: 50949163C/T
2-3: 50961828T/A
2-3: 50962069C/T
2-3: 50964721G/A
2-3: 50973099A/G
2-3: 51010771G/A
2-3: 51039435C/T
2-3: 51050625G/A
2-3: 51091434G/A
2-3: 51117472G/T
2-3: 51154366T/A
2-3: 51202354G/T
2-3: 51214750T/G
2-3: 51235131G/A
2-3: 51316647C/A
2-3: 51387778C/T
2-3: 51465978C/A
2-3: 51465987A/G
2-3: 51480824A/G
2-3: 51522969G/A
2-3: 51568205C/T
2-3: 51617668G/A
2-3: 51668673C/T
2-3: 51668760A/.
2-3: 51700620G/A
2-3: 51880015C/T
2-3: 51900285A/G
2-3: 51921268G/C
2-3: 51938055A/T
2-3: 51966597T/C
2-3: 51995888T/A
2-3: 52018691G/A
2-3: 52134660C/T
2-3: 52168186G/A
2-3: 52247126A/T
2-3: 52351495A/G
2-3: 52419058C/T
2-3: 52431135G/A
2-3: 52451459T/G
2-3: 52479028T/G
2-3: 52534739C/A
2-3: 52582644C/T
2-3: 52614717C/T
2-3: 52685911A/T
2-3: 52762262A/G
2-3: 52798394T/G
2-3: 52800580C/T
2-3: 52828241C/T
2-3: 52901540G/C
2-3: 52955498G/A
2-3: 52998118T/G
2-3: 53010950C/T
2-3: 53024198T/C
2-3: 53062820C/A
2-3: 53066387G/C
2-3: 53153620C/T
2-3: 53201610C/T
2-3: 53232416G/T
2-3: 53351906T/C
2-3: 53483374T/A
2-3: 53561665C/G
2-3: 53578590G/A
2-3: 53658220T/A
2-3: 53769227T/C
2-3: 53791254C/T
2-3: 53891578T/A
2-3: 53891781G/A
2-3: 53892871T/C
2-3: 53892932T/C
2-3: 53938740A/T
2-3: 53971556C/T
2-3: 53971678T/C
2-3: 53972056C/T
2-3: 53989944T/C
2-3: 54054096T/G
2-3: 54065264C/A
2-3: 54085282C/T
2-3: 54099670C/T
2-3: 54122377C/A
2-3: 54155382T/C
2-3: 54213821G/A
2-3: 54244584C/T
2-3: 54288546A/G
2-3: 54305693A/G
2-3: 54329092T/C
2-3: 54371772A/G
2-3: 54394617T/C
2-3: 54414479C/T
2-3: 54415083T/A
2-3: 54415389C/G
2-3: 54417618G/T
2-3: 54417955C/T
2-3: 54418394C/T
2-3: 54418402C/A
2-3: 54545237T/G
2-3: 54649992T/G
2-3: 54675764G/A
2-3: 54740801G/A
2-3: 54780401A/T
2-3: 54843732T/C
2-3: 54856495T/A
2-3: 54880100A/C
2-3: 54891580A/G
2-3: 54960999T/C
2-3: 54988012T/C
2-3: 55027756A/T
2-3: 55087153G/T
2-3: 55184950T/C
2-3: 55215745G/T
2-3: 55257668A/C
2-3: 55276270A/T
2-3: 55276427G/A
2-3: 55276774T/C
2-3: 55307219T/C
2-3: 55342305G/A
2-3: 55359196G/A
2-3: 55571018T/A
2-3: 55755660T/G
2-3: 55796977G/T
2-3: 55837348C/T
2-3: 55873905G/C
2-3: 55954207A/G
2-3: 55971877A/C
2-3: 56015138A/G
2-3: 56035432G/A
2-3: 56048787C/G
2-3: 56068673G/T
2-3: 56070305T/C
2-3: 56082988G/A
2-3: 56094623G/A
2-3: 56127090T/C
2-3: 56162368C/T
2-3: 56194164T/C
2-3: 56294071A/C
2-3: 56322444A/G
2-3: 56350760T/C
2-3: 56376137C/T
2-3: 56421175G/A
2-3: 56529425C/T
2-3: 56536302T/C
2-3: 56563916T/C
2-3: 56652637G/T
2-3: 56776773G/A
2-3: 56778963A/G
2-3: 56780650T/A
2-3: 56780906T/A
2-3: 56787102A/G
2-3: 56813656C/A
2-3: 56839684G/A
2-3: 56880638T/C
2-3: 56917380T/C
2-3: 56971420A/T
2-3: 57090637A/T
2-3: 57091410A/T
2-3: 57091607C/A
2-3: 57091640A/G
2-3: 57091796C/T
2-3: 57094330G/C
2-3: 57094343A/G
2-3: 57094803T/A
2-3: 57256975C/T
2-3: 57258078C/A
2-3: 57273519A/G
2-3: 57291114G/A
2-3: 57330919C/A
2-3: 57406333T/C
2-3: 57495045G/A
2-3: 57542640C/T
2-3: 57571704G/A
2-3: 57598509G/A
2-3: 57635604A/T
2-3: 57665086C/A
2-3: 57731007C/T
2-3: 57812247C/T
2-3: 57837652C/G
2-3: 57876298T/C
2-3: 57914781T/C
2-3: 57933356A/G
2-3: 58035326T/C
2-3: 58139655A/T
2-3: 58311736A/G
2-3: 58365907C/T
2-3: 58378827T/C
2-3: 58418455C/T
2-3: 58434514A/T
2-3: 58506326G/A
2-3: 58538327C/A
2-3: 58577791G/A
2-3: 58578366C/A
2-3: 58751234A/T
2-3: 58889214T/A
2-3: 58922854T/A
2-3: 58923817A/G
2-3: 58924124A/T
2-3: 58924620C/T
2-3: 58924967A/G
2-3: 58925757C/T
2-3: 58926619T/C
2-3: 58938098C/T
2-3: 59025971C/T
2-3: 59062770G/A
2-3: 59168293G/A
2-3: 59244158A/G
2-3: 59283267T/A
2-3: 59333039A/G
2-3: 59352843C/A
2-3: 59408615G/A
2-3: 59561837C/G
2-3: 59627404T/G
2-3: 59654947C/T
2-3: 59711902A/T
2-3: 59853046G/A
2-3: 59866788C/A
2-3: 59892420C/T
2-3: 59918228C/A
2-3: 59934017G/A
2-3: 59948245T/C
2-3: 60096085T/C
2-3: 60121853G/T
2-3: 60132321G/A
2-3: 60143864G/A
2-3: 60199338A/G
2-3: 60268169T/C
2-3: 60303125C/T
2-3: 60375459C/T
2-3: 60375988T/C
2-3: 60418706T/A
2-3: 60529301C/A
2-3: 60573647A/T
2-3: 60607816T/A
2-3: 60690896A/G
2-3: 60737885T/A
2-3: 60785077C/T
2-3: 60847309T/A
2-3: 60889257A/C
2-3: 60920019C/T
2-3: 60998779T/C
2-3: 61000048A/G
2-3: 61079883A/T
2-3: 61105406A/C
2-3: 61148189T/C
2-3: 61159979G/C
2-3: 61196587A/T
2-3: 61310789C/A
2-3: 61341284G/A
2-3: 61428677G/A
2-3: 61511867T/C
2-3: 61537246A/G
2-3: 61560728C/T
2-3: 61660806A/C
2-3: 61662261A/T
2-3: 61681694G/A
2-3: 61708426A/T
2-3: 61713541T/A
2-3: 61729888C/T
2-3: 61750794A/C
2-3: 61790642A/T
2-3: 61823502C/T
2-3: 61849293T/A
2-3: 61863659A/G
2-3: 62166223T/G
2-3: 62185486C/A
2-3: 62367143T/A
2-3: 62429489C/T
2-3: 62439493A/G
2-3: 62456602G/A
2-3: 62649685C/T
2-3: 62737571T/A
2-3: 62939071C/T
2-3: 62943769C/A
2-3: 63014625T/C
2-3: 63068550A/G
2-3: 63093081T/C
2-3: 63120321C/T
2-3: 63164669C/T
2-3: 63266403T/C
2-3: 63332757G/A
2-3: 63332766G/A
2-3: 63525193C/A
2-3: 63528930G/A
2-3: 63558877G/C
2-3: 63571067T/A
2-3: 63600059G/A
2-3: 63654003A/G
2-3: 63675876A/G
2-3: 63690308A/G
2-3: 63718763T/C
2-3: 63790418G/A
2-3: 63801803C/T
2-3: 63845571G/C
2-3: 63864914A/T
2-3: 64015258A/G
2-3: 64539445T/C
2-3: 64690089T/A
2-3: 64763516A/G
2-3: 64902542T/C
2-3: 64905598G/A
2-3: 64906706G/A
2-3: 64907391T/C
2-3: 64908026T/C
2-3: 64908493A/G
2-3: 64908613G/A
2-3: 64908984C/T
2-3: 64914167C/T
2-3: 64914635C/T
2-3: 64914839A/T
2-3: 64915191C/G
2-3: 64915671C/A
2-3: 64915790G/A
2-3: 64916528A/G
2-3: 64916631T/C
2-3: 64919624T/G
2-3: 64933354C/G
2-3: 64935514C/T
2-3: 64935928A/T
2-3: 64936050G/A
2-3: 64936661C/T
2-3: 64937519C/G
2-3: 64937722A/G
2-3: 64937736G/A
2-3: 64937873C/T
2-3: 64942970C/T
2-3: 64943138T/C
2-3: 64944529G/T
2-3: 64945693T/C
2-3: 64945876C/A
2-3: 64946398A/G
2-3: 64946592T/C
2-3: 64948906C/T
2-3: 64949802C/G
2-3: 64950688G/A
2-3: 64950845A/G
2-3: 64953440A/T
2-3: 64954868T/G
2-3: 64959002C/A
2-3: 64959254T/C
2-3: 64959408A/G
2-3: 64959773C/T
2-3: 64960115A/G
2-3: 64960229T/A
2-3: 64960251G/T
2-3: 64960891C/A
2-3: 64961088G/C
2-3: 64961783G/A
2-3: 64961897C/T
2-3: 64962195A/T
2-3: 64962345C/T
2-3: 64971168G/A
2-3: 65019971T/C
2-3: 65027025C/T
2-3: 65027947G/A
2-3: 65041903A/G
2-3: 65098538T/A
2-3: 65126315G/A
2-3: 65258663A/T
2-3: 65322935T/A
2-3: 65396318C/T
2-3: 65441513G/A
2-3: 65453885C/T
2-3: 65576947C/T
2-3: 65602527C/T
2-3: 65688119A/C
2-3: 65751794C/T
2-3: 65757500C/G
2-3: 66138298C/T
2-3: 66271916A/G
2-3: 66276278G/T
2-3: 66276295C/T
2-3: 66348829C/T
2-3: 66430965T/C
2-3: 66542215C/T
2-4: 113856T/C
2-4: 132262T/A
2-4: 146042C/T
2-4: 374413T/C
2-4: 381787A/G
2-4: 481443T/G
2-4: 589546G/A
2-4: 676945A/G
2-4: 688274G/T
2-4: 854825G/A
2-4: 941983G/C
2-4: 1112501C/A
2-4: 1557731G/A
2-4: 1876727T/C
2-4: 2004299C/T
2-4: 2004365T/G
2-4: 2010377T/C
2-4: 2010705G/A
2-4: 2010708A/G
2-4: 2010715A/G
2-4: 2141350G/A
2-4: 2153513T/G
2-4: 2451876C/T
2-4: 2515219C/T
2-4: 2540677C/A
2-4: 2695908A/G
2-4: 2744941C/T
2-4: 2856980C/A
2-4: 2863723T/C
2-4: 2890977T/G
2-4: 2893371C/A
2-4: 2893640T/C
2-4: 2894155A/G
2-4: 2902752T/C
2-4: 2918625C/A
2-4: 3063784C/A
2-4: 3222278T/A
2-4: 3320992C/T
2-4: 3332496T/A
2-4: 3479886G/A
2-4: 3598759C/T
2-4: 3863574G/A
2-4: 3962863T/C
2-4: 3996051C/A
2-4: 4083670T/C
2-4: 4224993G/A
2-4: 4235351G/A
2-4: 4276610T/C
2-4: 4312215T/G
2-4: 4342859T/A
2-4: 4342879T/C
2-4: 4342895T/A
2-4: 4343330G/T
2-4: 4600574A/G
2-4: 4650577G/A
2-4: 4806381G/A
2-4: 4878313T/C
2-4: 4895408C/A
2-4: 4996681A/G
2-4: 5067522G/A
2-4: 5346764G/A
2-4: 5395616T/C
2-4: 5491831G/T
2-4: 5704883T/C
2-4: 5774846T/C
2-4: 5876573G/C
2-4: 5889061C/T
2-4: 5905997A/G
2-4: 5930324A/G
2-4: 5934568T/C
2-4: 5934714G/A
2-4: 5940627T/C
2-4: 5962778C/G
2-4: 5992093C/T
2-4: 6022952G/A
2-4: 6091010G/C
2-4: 6131787G/A
2-4: 6131927C/T
2-4: 6156040A/G
2-4: 6172236T/C
2-4: 6304250T/G
2-4: 6367593C/T
2-4: 6482092A/T
2-4: 6520748T/G
2-4: 6557151A/G
2-4: 6609741C/A
2-4: 6718004C/T
2-4: 6765948C/G
2-4: 6842356A/G
2-4: 6922620A/G
2-4: 6986433C/T
2-4: 7024662A/T
2-4: 7061131C/T
2-4: 7168723T/C
2-4: 7243549G/A
2-4: 7250858T/A
2-4: 7286822C/T
2-4: 7313688C/T
2-4: 7328023A/T
2-4: 7364236A/T
2-4: 7432611G/T
2-4: 7438005A/C
2-4: 7457909A/G
2-4: 7489959T/C
2-4: 7520321T/A
2-4: 7562046C/T
2-4: 7568512T/C
2-4: 7569117T/G
2-4: 7569577G/A
2-4: 7569689C/G
2-4: 7572240G/A
2-4: 7622769C/T
2-4: 7643359C/T
2-4: 7690350G/A
2-4: 7829626T/A
2-4: 7884003C/T
2-4: 7944005T/A
2-4: 7958170A/G
2-4: 8000762A/G
2-4: 8076585G/C
2-4: 8107015C/T
2-4: 8233271G/A
2-4: 8284682G/A
2-4: 8331150T/C
2-4: 8363292T/C
2-4: 8406758G/C
2-4: 8514951T/C
2-4: 8545107A/G
2-4: 8587336A/G
2-4: 8727151A/G
2-4: 8730091C/A
2-4: 8730811G/A
2-4: 8773468C/T
2-4: 8827688C/T
2-4: 8837971G/T
2-4: 8838158T/C
2-4: 8840280T/C
2-4: 8840434C/A
2-4: 8840714A/T
2-4: 8841790C/G
2-4: 8841897T/C
2-4: 8841900G/A
2-4: 8873309T/C
2-4: 8926901C/A
2-4: 9018018C/T
2-4: 9029688G/C
2-4: 9095389C/T
2-4: 9222103T/C
2-4: 9237377T/A
2-4: 9260165T/A
2-4: 9277685G/T
2-4: 9357176G/A
2-4: 9382764T/C
2-4: 9469376T/C
2-4: 9516951T/C
2-4: 9599409T/C
2-4: 9628271T/C
2-4: 9675134T/G
2-4: 9762242G/A
2-4: 9789167G/A
2-4: 9801020T/C
2-4: 9802398G/A
2-4: 9823109C/T
2-4: 9865789G/C
2-4: 9948268A/G
2-4: 9962021A/G
2-4: 10013435A/T
2-4: 10015717G/T
2-4: 10059904C/T
2-4: 10189179A/G
2-4: 10264408G/T
2-4: 10291524C/T
2-4: 10404393T/C
2-4: 10409246G/A
2-4: 10465534G/A
2-4: 10478571T/A
2-4: 10507912A/T
2-4: 10689906A/G
2-4: 10835720T/A
2-4: 10856762C/A
2-4: 10857659C/T
2-4: 10901517A/T
2-4: 10945099A/T
2-4: 10981259G/A
2-4: 11006318T/C
2-4: 11151459G/A
2-4: 11210060A/G
2-4: 11325594A/T
2-4: 11374972G/A
2-4: 11420854G/A
2-4: 11454428A/G
2-4: 11486150G/A
2-4: 11503317C/T
2-4: 11597051T/A
2-4: 11771622A/T
2-4: 11858239A/G
2-4: 12039423A/T
2-4: 12052708G/T
2-4: 12205143T/C
2-4: 12265068T/C
2-4: 12266359A/C
2-4: 12375803G/C
2-4: 12395869T/C
2-4: 12454354C/A
2-4: 12486210C/A
2-4: 12544502A/T
2-4: 12570012C/T
2-4: 12627651C/A
2-4: 12723817C/T
2-4: 12777354T/G
2-4: 12814298C/T
2-4: 12840527G/C
2-4: 12915322C/A
2-4: 12928704C/T
2-4: 12986224A/G
2-4: 13002326C/T
2-4: 13051652T/C
2-4: 13078785G/A
2-4: 13128207A/T
2-4: 13128411A/G
2-4: 13157166C/G
2-4: 13176830C/A
2-4: 13192195A/C
2-4: 13208278C/A
2-4: 13295996T/G
2-4: 13410843C/T
2-4: 13414575T/C
2-4: 13559388G/A
2-4: 13624622T/C
2-4: 13636704G/A
2-4: 13637309C/T
2-4: 13653227T/G
2-4: 13654847A/T
2-4: 13679902A/G
2-4: 13727075T/C
2-4: 13863889G/A
2-4: 13864985G/A
2-4: 13932283G/T
2-4: 14065575A/G
2-4: 14104828C/T
2-4: 14178112C/G
2-4: 14214589T/A
2-4: 14305401C/T
2-4: 14430990G/A
2-4: 14439893A/G
2-4: 14564069T/A
2-4: 14651873C/G
2-4: 14669856T/C
2-4: 14685623T/C
2-4: 14699167G/A
2-4: 14719107A/G
2-4: 14746714A/C
2-4: 14761408A/T
2-4: 14761575C/T
2-4: 14780516T/G
2-4: 14809069G/A
2-4: 14864149C/A
2-4: 14876186G/C
2-4: 14886478T/C
2-4: 14897241A/T
2-4: 14920609G/A
2-4: 15039616C/T
2-4: 15061916G/A
2-4: 15085730A/G
2-4: 15151474A/G
2-4: 15152985A/G
2-4: 15209454T/G
2-4: 15245050T/C
2-4: 15255383T/C
2-4: 15346122T/A
2-4: 15372106G/A
2-4: 15432523T/G
2-4: 15474656T/C
2-4: 15573823G/A
2-4: 15681439T/G
2-4: 15691286C/T
2-4: 15743699C/A
2-4: 15771413G/A
2-4: 15805161A/G
2-4: 15822067G/A
2-4: 15833698G/T
2-4: 15943604T/A
2-4: 15982317T/G
2-4: 16163881T/A
2-4: 16167123T/C
2-4: 16206702C/T
2-4: 16273653C/T
2-4: 16310517T/A
2-4: 16345873T/C
2-4: 16401553T/A
2-4: 16490915T/C
2-4: 16610663A/T
2-4: 16754581T/A
2-4: 16761472T/C
2-4: 16876327A/C
2-4: 16975672T/C
2-4: 17030890G/A
2-4: 17084895A/C
2-4: 17197440T/C
2-4: 17286313G/A
2-4: 17446478A/T
2-4: 17522416T/C
2-4: 17535478C/T
2-4: 17560574T/C
2-4: 17636940T/A
2-4: 17808962T/C
2-4: 17823890A/T
2-4: 17870245C/T
2-4: 17929543C/T
2-4: 17943641G/C
2-4: 17963388C/T
2-4: 17979737C/A
2-4: 17994659T/C
2-4: 18022313A/C
2-4: 18087748G/T
2-4: 18087751C/A
2-4: 18087860G/C
2-4: 18087883G/T
2-4: 18087958T/C
2-4: 18088001G/A
2-4: 18088005G/C
2-4: 18088078C/A
2-4: 18093366T/C
2-4: 18100281A/T
2-4: 18100338C/T
2-4: 18101021T/C
2-4: 18101039G/A
2-4: 18101093A/T
2-4: 18101128G/A
2-4: 18101890T/.
2-4: 18108182G/A
2-4: 18119724C/T
2-4: 18231777A/T
2-4: 18242554C/G
2-4: 18265013C/T
2-4: 18373789T/C
2-4: 18392329G/A
2-4: 18431858T/C
2-4: 18461773C/T
2-4: 18504599G/T
2-4: 18569879G/A
2-4: 18602733A/T
2-4: 18663703T/A
2-4: 18693197C/T
2-4: 18713596A/C
2-4: 18873150G/T
2-4: 18956888T/G
2-4: 19021443T/C
2-4: 19053596C/T
2-4: 19123485T/A
2-4: 19153539T/C
2-4: 19243755C/G
2-4: 19258795G/T
2-4: 19260739G/C
2-4: 19269376T/C
2-4: 19280366A/G
2-4: 19299385A/G
2-4: 19313905A/G
2-4: 19315655T/C
2-4: 19326986T/G
2-4: 19356317T/C
2-4: 19368172C/T
2-4: 19398250T/C
2-4: 19431924G/T
2-4: 19516364A/T
2-4: 19546609C/G
2-4: 19584407G/A
2-4: 19609465A/G
2-4: 19680645G/A
2-4: 19732300G/A
2-4: 19738140C/A
2-4: 19816002T/C
2-4: 19891429T/G
2-4: 19930036T/C
2-4: 19960446A/G
2-4: 20014546C/T
2-4: 20044675A/C
2-4: 20093700A/G
2-4: 20217915G/A
2-4: 20321978A/T
2-4: 20381205G/T
2-4: 20411708T/.
2-4: 20411939C/.
2-4: 20412421C/.
2-4: 20412478G/.
2-4: 20412600G/.
2-4: 20432614T/C
2-4: 20442795A/C
2-4: 20482264T/G
2-4: 20509453G/A
2-4: 20558397T/G
2-4: 20559676C/A
2-4: 20559724C/T
2-4: 20587089A/T
2-4: 20622163T/G
2-4: 20654671C/T
2-4: 20689235T/A
2-4: 20718858C/T
2-4: 20744275G/A
2-4: 20782986G/C
2-4: 20877002C/T
2-4: 20934660T/A
2-4: 20974505T/C
2-4: 21025402T/A
2-4: 21195205T/C
2-4: 21360331A/G
2-4: 21436105T/C
2-4: 21452642G/A
2-4: 21490263G/A
2-4: 21534409C/T
2-4: 21621840G/T
2-4: 21702567C/T
2-4: 21790929T/G
2-4: 21856935C/T
2-4: 21920193C/T
2-4: 21951670G/A
2-4: 21989172A/G
2-4: 21992924C/T
2-4: 21993047C/T
2-4: 21993049G/A
2-4: 21993270G/A
2-4: 21993375T/C
2-4: 22009950G/C
2-4: 22018601G/C
2-4: 22019232G/T
2-4: 22019280A/G
2-4: 22019400G/A
2-4: 22020475T/C
2-4: 22032928C/T
2-4: 22045713C/A
2-4: 22070161A/C
2-4: 22070275G/A
2-4: 22070291G/A
2-4: 22127843C/T
2-4: 22128075A/G
2-4: 22128786G/A
2-4: 22271624A/G
2-4: 22397705A/T
2-4: 22428467C/T
2-4: 22429185T/C
2-4: 22445336A/C
2-4: 22455945A/T
2-4: 22491266G/A
2-4: 22521453A/G
2-4: 22531307G/A
2-4: 22532457T/C
2-4: 22550564C/A
2-4: 22579087C/T
2-4: 22667489A/C
2-4: 22670954C/G
2-4: 22728854G/A
2-4: 22729654C/T
2-4: 22744482C/T
2-4: 22802607T/A
2-4: 22831546G/A
2-4: 22890089C/T
2-4: 22965059T/C
2-4: 22976841A/G
2-4: 22987396G/A
2-4: 23021554C/T
2-4: 23057759G/T
2-4: 23091359G/A
2-4: 23098907T/C
2-4: 23100514T/G
2-4: 23111096A/G
2-4: 23130768T/C
2-4: 23147022C/T
2-4: 23183652T/A
2-4: 23211229A/G
2-4: 23271363T/G
2-4: 23288853A/G
2-4: 23318287T/C
2-4: 23319855G/A
2-4: 23332201A/G
2-4: 23336546C/A
2-4: 23464681T/C
2-4: 23560388T/C
2-4: 23669603C/A
2-4: 23769522A/G
2-4: 23777728A/G
2-4: 23864479G/T
2-4: 23995997A/T
2-4: 24031186A/G
2-4: 24119589T/A
2-4: 24205826A/G
2-4: 24226493T/C
2-4: 24292958A/G
2-4: 24303301C/T
2-4: 24309657A/G
2-4: 24309687T/G
2-4: 24309821A/G
2-4: 24310093G/T
2-4: 24314912G/A
2-4: 24399424C/T
2-4: 24531437A/C
2-4: 24653361C/T
2-4: 24718212G/A
2-4: 24757507A/G
2-4: 24760808C/T
2-4: 24761153T/C
2-4: 24877920T/C
2-4: 25018032C/T
2-4: 25018501C/T
2-4: 25115448A/G
2-4: 25328127T/C
2-4: 25447103T/C
2-4: 25508997T/C
2-4: 25599307C/A
2-4: 25674421T/C
2-4: 25697620A/T
2-4: 25700818C/T
2-4: 25790692G/A
2-4: 25883323T/C
2-4: 25886939C/T
2-4: 25940691G/A
2-4: 25984315G/A
2-4: 26018608T/C
2-4: 26052426C/T
2-4: 26084512T/C
2-4: 26136041G/A
2-4: 26222727T/C
2-4: 26253111C/T
2-4: 26313393C/G
2-4: 26363857C/T
2-4: 26414487A/T
2-4: 26429474T/C
2-4: 26458885A/G
2-4: 26495691T/C
2-4: 26515441C/T
2-4: 26552075G/A
2-4: 26565960A/C
2-4: 26591243A/G
2-4: 26591953A/G
2-4: 26653736T/C
2-4: 26719842C/T
2-4: 26745716A/G
2-4: 26791346G/A
2-4: 26825709C/T
2-4: 26866036G/A
2-4: 26893596T/C
2-4: 26940644T/C
2-4: 27046250C/A
2-4: 27089314G/A
2-4: 27222602A/C
2-4: 27241060C/G
2-4: 27256645T/C
2-4: 27369766A/C
2-4: 27581582C/T
2-4: 27623841A/G
2-4: 27777053A/T
2-4: 27832169G/C
2-4: 27884708C/T
2-4: 28035065A/T
2-4: 28165419G/A
2-4: 28210157A/G
2-4: 28240944C/T
2-4: 28341191T/G
2-4: 28409328A/G
2-4: 28450490C/T
2-4: 28526489G/A
2-4: 28594182G/A
2-4: 28625973T/A
2-4: 28749025G/A
2-4: 28759308T/C
2-4: 28816740G/A
2-4: 28934847A/C
2-4: 29030468T/C
2-4: 29179687A/T
2-4: 29194140A/G
2-4: 29251271A/G
2-4: 29251826T/A
2-4: 29251939C/T
2-4: 29276283A/T
2-4: 29370142A/G
2-4: 29392086G/A
2-4: 29397254C/A
2-4: 29439772C/G
2-4: 29440970C/T
2-4: 29456743C/A
2-4: 29485902A/G
2-4: 29493930T/C
2-4: 29494146G/A
2-4: 29494306G/A
2-4: 29498441T/A
2-4: 29521816A/G
2-4: 29540207A/C
2-4: 29572422T/G
2-4: 29582617G/A
2-4: 29701589T/G
2-4: 29706549C/T
2-4: 29711937A/T
2-4: 29745762G/A
2-4: 29776668G/A
2-4: 29822006C/T
2-4: 29866852T/C
2-4: 29897105T/G
2-4: 29972673C/T
2-4: 30114435T/C
2-4: 30148674A/G
2-4: 30185867G/T
2-4: 30216632G/A
2-4: 30288939T/C
2-4: 30326175G/T
2-4: 30372456A/C
2-4: 30443954A/C
2-4: 30473338T/A
2-4: 30542726G/T
2-4: 30544620T/G
2-4: 30544723A/T
2-4: 30559115C/T
2-4: 30563475A/G
2-4: 30582102G/T
2-4: 30612949A/T
2-4: 30665420G/A
2-4: 30675559C/T
2-4: 30675722A/G
2-4: 30682473G/A
2-4: 30705199A/G
2-4: 30729747A/G
2-4: 30753894A/G
2-4: 30780084T/A
2-4: 30792701G/T
2-4: 30847254T/A
2-4: 30929207C/A
2-4: 30987223G/A
2-4: 30993198C/T
2-4: 31026256T/C
2-4: 31051589G/T
2-4: 31067439G/C
2-4: 31072184A/G
2-4: 31154718A/G
2-4: 31195496G/A
2-4: 31222902A/G
2-4: 31237775A/T
2-4: 31293163G/A
2-4: 31321954G/A
2-4: 31324952T/G
2-4: 31325654A/G
2-4: 31353933C/T
2-4: 31384216A/T
2-4: 31554662T/G
2-4: 31554788T/G
2-4: 31562290A/T
2-4: 31562703A/G
2-4: 31563017G/A
2-4: 31679689T/C
2-4: 31702967T/C
2-4: 31954059C/T
2-4: 32012588A/G
2-4: 32059730C/T
2-4: 32220216G/T
2-4: 32319116T/C
2-4: 32332046C/A
2-4: 32370006A/G
2-4: 32400072A/C
2-4: 32423107A/G
2-4: 32459033G/A
2-4: 32479242G/A
2-4: 32531485T/C
2-4: 32576611A/G
2-4: 32591508C/T
2-4: 32602340C/T
2-4: 32632940A/G
2-4: 32750954C/T
2-4: 32767386G/A
2-4: 32831234G/A
2-4: 32858033A/T
2-4: 32911989C/T
2-4: 32942880C/T
2-4: 32976935A/G
2-4: 32984275A/T
2-4: 33033328T/C
2-4: 33045838A/G
2-4: 33189660A/G
2-4: 33200206G/T
2-4: 33222458C/T
2-4: 33260078T/C
2-4: 33285511G/A
2-4: 33321577A/G
2-4: 33361082T/C
2-4: 33420271G/A
2-4: 33489109T/G
2-4: 33504251T/A
2-4: 33516157G/A
2-4: 33593153T/C
2-4: 33609247A/C
2-4: 33621349A/T
2-4: 33633108C/G
2-4: 33650373T/C
2-4: 33720456G/C
2-4: 33805667G/C
2-4: 33845720T/C
2-4: 33878854C/T
2-4: 33892191A/T
2-4: 33911871G/A
2-4: 33916352C/A
2-4: 33916868G/T
2-4: 33916942G/T
2-4: 33932462G/C
2-4: 33966020A/G
2-4: 34046557C/T
2-4: 34090234A/T
2-4: 34167309G/A
2-4: 34272351A/T
2-4: 34289514T/C
2-4: 34316191C/T
2-4: 34375226C/T
2-4: 34405215T/C
2-4: 34449586C/A
2-4: 34461259T/A
2-4: 34565022A/T
2-4: 34649596G/A
2-4: 34661416T/C
2-4: 34822328A/G
2-4: 34837048T/C
2-4: 34962801A/G
2-4: 34967663C/T
2-4: 35076659G/A
2-4: 35123993T/C
2-4: 35286551G/T
2-4: 35383056T/A
2-4: 35384141T/C
2-4: 35397396G/A
2-4: 35472490G/T
2-4: 35691328A/T
2-4: 35770378C/A
2-4: 35791871G/A
2-4: 35829356A/G
2-4: 35829453C/T
2-4: 35944088A/G
2-4: 36038224G/A
2-4: 36110200G/C
2-4: 36111644T/C
2-4: 36125824A/G
2-4: 36197368C/T
2-4: 36197784A/C
2-4: 36262619A/G
2-4: 36323513T/C
2-4: 36459086A/C
2-4: 36491764T/C
2-4: 36512699C/T
2-4: 36628599A/C
2-4: 36757948A/T
2-4: 36814482C/T
2-4: 36870770G/A
2-4: 36930522A/G
2-4: 36956252A/G
2-4: 36975291G/A
2-4: 37011826T/C
2-4: 37029345G/A
2-4: 37178181G/A
2-4: 37178595C/G
2-4: 37182698C/A
2-4: 37185794G/A
2-4: 37187068T/C
2-4: 37188078C/G
2-4: 37330732C/T
2-4: 37402202T/C
2-4: 37493020A/G
2-4: 37595074C/T
2-4: 37596772C/G
2-4: 37609321A/C
2-4: 37639085C/T
2-4: 37643432A/G
2-4: 37650492C/T
2-4: 37661906T/G
2-4: 37717487A/G
2-4: 37747022C/T
2-4: 37867882G/A
2-4: 37896632A/T
2-4: 37933056A/C
2-4: 37973104C/G
2-4: 38050459T/A
2-4: 38081466C/A
2-4: 38169572A/G
2-4: 38198124T/C
2-4: 38250511C/T
2-4: 38278038T/C
2-4: 38326497C/T
2-4: 38374099C/T
2-4: 38420420C/A
2-4: 38468475A/T
2-4: 38474163C/A
2-4: 38561929T/A
2-4: 38613319C/T
2-4: 38719691C/T
2-4: 38720164T/G
2-4: 38726390A/C
2-4: 38740699G/A
2-4: 38837238C/T
2-4: 38843602C/G
2-4: 38852614G/A
2-4: 38890459T/C
2-4: 38915226C/T
2-4: 39007684A/G
2-4: 39082266G/T
2-4: 39095771T/C
2-4: 39157869T/C
2-4: 39169968C/A
2-4: 39234667A/C
2-4: 39277431C/T
2-4: 39367733T/G
2-4: 39394513C/T
2-4: 39449070G/A
2-4: 39467781C/T
2-4: 39518712T/A
2-4: 39574577A/G
2-4: 39587749A/G
2-4: 39601343G/T
2-4: 39645847C/G
2-4: 39646515T/A
2-4: 39696974T/A
2-4: 39739593G/A
2-4: 39792941A/G
2-4: 39804270C/A
2-4: 39828820T/A
2-4: 39842596C/T
2-4: 39842705G/C
2-4: 39843111A/C
2-4: 39847394G/C
2-4: 39847860G/T
2-4: 39850090C/A
2-4: 39861373C/T
2-4: 39872341T/C
2-4: 39877108C/T
2-4: 39877360C/T
2-4: 39880818T/C
2-4: 39882308T/A
2-4: 39884649C/T
2-4: 39888866T/C
2-4: 39937042G/A
2-4: 40002265T/C
2-4: 40202304A/T
2-4: 40418197T/G
2-4: 40466561C/T
2-4: 40571660G/T
2-4: 40645826G/A
2-4: 40781864C/T
2-4: 40886027A/T
2-4: 40888238G/T
2-4: 40921059G/T
2-4: 41022882T/C
2-4: 41031630C/T
2-4: 41048077A/T
2-4: 41090741A/C
2-4: 41102413G/A
2-4: 41160619A/G
2-4: 41193762G/A
2-4: 41204166T/C
2-4: 41333728C/G
2-4: 41373750C/T
2-4: 41380088C/T
2-4: 41393310G/A
2-4: 41407502A/C
2-4: 41446841A/T
2-4: 41490967T/C
2-4: 41518824C/G
2-4: 41533470T/C
2-4: 41567420A/G
2-4: 41611310A/G
2-4: 41659119A/G
2-4: 41707657C/G
2-4: 41708610G/A
2-4: 41738781T/G
2-4: 41842636A/T
2-4: 41859728C/T
2-4: 41865311G/A
2-4: 41885186C/T
2-4: 41965648C/G
2-4: 41978776A/G
2-4: 42080388T/A
2-4: 42129657C/T
2-4: 42143014C/T
2-4: 42157139T/G
2-4: 42168471C/T
2-4: 42220713A/G
2-4: 42221796G/A
2-4: 42223854C/T
2-4: 42223910G/T
2-4: 42223919C/T
2-4: 42266942C/T
2-4: 42300196A/G
2-4: 42310938G/C
2-4: 42333016C/T
2-4: 42377338T/C
2-4: 42441736T/C
2-4: 42474386T/G
2-4: 42496443T/C
2-4: 42523308A/G
2-4: 42523313A/G
2-4: 42531505C/A
2-4: 42531633C/T
2-4: 42539469G/A
2-4: 42559182A/G
2-4: 42608154G/T
2-4: 42685259A/G
2-4: 42791683G/A
2-4: 42793385T/C
2-4: 42804958G/A
2-4: 42806507G/A
2-4: 42845983G/A
2-4: 42880098A/G
2-4: 42931262A/G
2-4: 42945666A/T
2-4: 42958115G/A
2-4: 42984922A/C
2-4: 43012815A/C
2-4: 43032039T/A
2-4: 43047120C/A
2-4: 43060661G/A
2-4: 43093549A/G
2-4: 43144521C/T
2-4: 43196506C/T
2-4: 43221755C/A
2-4: 43338176T/A
2-4: 43451525A/G
2-4: 43480053A/G
2-4: 43526967A/G
2-4: 43531456T/G
2-4: 43531901T/C
2-4: 43538429T/C
2-4: 43550221C/T
2-4: 43578534A/T
2-4: 43642000C/T
2-4: 43669787G/C
2-4: 43707907G/A
2-4: 43734058T/C
2-4: 43741285T/G
2-4: 43744593T/C
2-4: 43745723T/C
2-4: 43746230T/G
2-4: 43748072A/G
2-4: 43748073A/G
2-4: 43748215G/A
2-4: 43748796G/A
2-4: 43749909A/G
2-4: 43749943G/T
2-4: 43750049A/G
2-4: 43755652T/C
2-4: 43782067G/A
2-4: 43837179C/T
2-4: 43850256G/C
2-4: 43886386A/G
2-4: 43900189G/A
2-4: 43916640T/A
2-4: 43990177A/G
2-4: 44004310A/T
2-4: 44023547T/C
2-4: 44051033G/A
2-4: 44080429A/G
2-4: 44106569C/T
2-4: 44118583G/A
2-4: 44142113C/T
2-4: 44142126T/C
2-4: 44152215G/A
2-4: 44155923C/T
2-4: 44176540G/A
2-4: 44192659A/T
2-4: 44271506G/T
2-4: 44271763A/G
2-4: 44301286G/A
2-4: 44301567A/G
2-4: 44303460A/G
2-4: 44303621T/A
2-4: 44303891A/G
2-4: 44310811G/A
2-4: 44311660G/A
2-4: 44322231T/G
2-4: 44328821T/A
2-4: 44335692T/C
2-4: 44336290G/A
2-4: 44336457A/G
2-4: 44336758C/G
2-4: 44336771G/T
2-4: 44347604G/A
2-4: 44381884C/A
2-4: 44395246G/C
2-4: 44427664G/A
2-4: 44445426A/G
2-4: 44458764T/C
2-4: 44469765T/A
2-4: 44498084A/T
2-4: 44575528C/A
2-4: 44619368C/T
2-4: 44635832T/G
2-4: 44654946T/G
2-4: 44711054T/C
2-4: 44768918C/T
2-4: 44806044A/T
2-4: 44878401T/C
2-4: 44915601G/A
2-4: 45000639T/A
2-4: 45041821C/T
2-4: 45303122A/G
2-4: 45307671A/T
2-4: 45396453T/C
2-4: 45435304A/G
2-4: 45507970A/C
2-4: 45605360C/A
2-4: 45605601T/C
2-4: 45605867C/T
2-4: 45605893C/T
2-4: 45605971T/G
2-4: 45606077G/C
2-4: 45628528G/C
2-4: 45635274T/C
2-4: 45636573G/A
2-4: 45636717C/G
2-4: 45637275G/T
2-4: 45637423G/A
2-4: 45637814G/T
2-4: 45637823T/G
2-4: 45637924C/T
2-4: 45638107G/T
2-4: 45638186C/T
2-4: 45638399C/T
2-4: 45638401C/T
2-4: 45638567G/C
2-4: 45638632C/T
2-4: 45638684T/G
2-4: 45638810C/A
2-4: 45638944T/C
2-4: 45638996T/C
2-4: 45639135T/G
2-4: 45639220G/T
2-4: 45639323T/C
2-4: 45639434A/C
2-4: 45667999A/T
2-4: 45668078G/A
2-4: 45668532T/A
2-4: 45668538G/T
2-4: 45669052G/A
2-4: 45669367G/A
2-4: 45669385G/A
2-4: 45669572A/G
2-4: 45669613C/T
2-4: 45669803A/G
2-4: 45670625A/C
2-4: 45670765A/T
2-4: 45671128A/G
2-4: 45671308A/T
2-4: 45671645G/C
2-4: 45671813C/A
2-4: 45672152G/A
2-4: 45672528G/A
2-4: 45672671A/C
2-4: 45673177A/G
2-4: 45707368G/C
2-4: 45718801T/G
2-4: 45789562G/T
2-4: 45799475C/T
2-4: 45805564G/A
2-4: 45813019C/T
2-4: 45845033A/G
2-4: 45860995G/T
2-4: 45881737T/A
2-4: 45968708G/A
2-4: 46006522T/A
2-4: 46008752T/C
2-4: 46008753C/T
2-4: 46016909T/C
2-4: 46037032G/A
2-4: 46044952C/T
2-4: 46050570G/T
2-4: 46083573G/A
2-4: 46092898T/C
2-4: 46133060T/C
2-4: 46185897T/C
2-4: 46202734T/A
2-4: 46292163A/C
2-4: 46292428T/C
2-4: 46313124T/A
2-4: 46368064G/A
2-4: 46385475G/A
2-4: 46400258A/G
2-4: 46446629T/A
2-4: 46451602C/A
2-4: 46498977A/G
2-4: 46509201C/T
2-4: 46513254G/A
2-4: 46515480T/C
2-4: 46540506G/A
2-4: 46552662T/C
2-4: 46552810T/C
2-4: 46552908C/T
2-4: 46560576A/G
2-4: 46560680A/C
2-4: 46562733A/C
2-4: 46562742G/A
2-4: 46563403A/G
2-4: 46574321A/G
2-4: 46592178C/T
2-4: 46649551T/C
2-4: 46708661T/G
2-4: 46750441T/C
2-4: 46762866T/A
2-4: 46821798G/.
2-4: 46875432C/A
2-4: 46930594A/G
2-4: 46930671G/T
2-4: 46936185G/T
2-4: 46955466G/A
2-4: 47004421A/T
2-4: 47039759A/T
2-4: 47051010T/C
2-4: 47051521A/C
2-4: 47053281T/C
2-4: 47053403C/A
2-4: 47068294G/A
2-4: 47081455C/T
2-4: 47158876T/A
2-4: 47189980A/G
2-4: 47253837A/G
2-4: 47307258T/A
2-4: 47375835C/T
2-4: 47467595A/G
2-4: 47502636C/T
2-4: 47554918A/G
2-4: 47624279C/T
2-4: 47655584C/T
2-4: 47685498T/C
2-4: 47700584C/T
2-4: 47715883A/T
2-4: 47720793G/T
2-4: 47768401C/T
2-4: 47821706A/G
2-4: 47846907G/T
2-4: 47910914A/G
2-4: 47963763A/G
2-4: 48037900A/G
2-4: 48038563C/G
2-4: 48041897C/T
2-4: 48076183G/T
2-4: 48109288G/A
2-4: 48121960G/T
2-4: 48141576C/A
2-4: 48363850A/G
2-4: 48390670T/G
2-4: 48410564T/G
2-4: 48438827C/T
2-4: 48453098A/T
2-4: 48468475A/G
2-4: 48583811A/G
2-4: 48638022T/G
2-4: 48749278T/C
2-4: 48794692G/A
2-4: 48841607T/A
2-4: 48861730A/G
2-4: 48918212T/A
2-4: 49086737G/A
2-4: 49184917A/G
2-4: 49223724T/C
2-4: 49275116T/C
2-4: 49348211C/A
2-4: 49365520C/T
2-4: 49434015T/A
2-4: 49488722A/G
2-4: 49528295C/T
2-4: 49564594T/A
2-4: 49596925G/T
2-4: 49603062G/A
2-4: 49620635A/G
2-4: 49626057C/T
2-4: 49628561G/A
2-4: 49657789T/A
2-4: 49730474A/G
2-4: 49763851G/T
2-4: 49776337A/G
2-4: 49820092C/T
2-4: 49820374T/C
2-4: 49827250T/C
2-4: 49835233T/C
2-4: 49858575C/A
2-4: 49884713A/G
2-4: 49891644C/T
2-4: 49923125G/T
2-4: 49970251G/A
2-4: 50007488T/A
2-4: 50027520A/G
2-4: 50045638G/A
2-4: 50073414T/C
2-4: 50086852G/T
2-4: 50099736A/G
2-4: 50105594C/T
2-4: 50105611C/A
2-4: 50106239G/T
2-4: 50124094T/C
2-4: 50168330C/T
2-4: 50257888C/T
2-4: 50268266C/T
2-4: 50296541T/A
2-4: 50378819C/T
2-4: 50517348T/C
2-4: 50541542A/T
2-4: 50553466T/C
2-4: 50606313G/A
2-4: 50620969A/T
2-4: 50628421T/A
2-4: 50682906G/A
2-4: 50698756G/A
2-4: 50717029T/G
2-4: 50733702G/A
2-4: 50764382A/G
2-4: 50770138C/G
2-4: 50815089A/G
2-4: 50854715G/T
2-4: 50865936A/G
2-4: 50865952T/C
2-4: 50891447A/G
2-4: 50921053A/T
2-4: 50932202T/A
2-4: 50948051A/G
2-4: 50960635C/T
2-4: 50961730G/A
2-4: 50989456C/T
2-4: 51001510A/G
2-4: 51004823G/T
2-4: 51011661T/C
2-4: 51040062G/T
2-4: 51050729A/G
2-4: 51061364A/G
2-4: 51077114C/T
2-4: 51088195C/T
2-4: 51115598C/T
2-4: 51151247T/C
2-4: 51177031G/C
2-4: 51231705C/T
2-4: 51243451T/C
2-4: 51262760C/A
2-4: 51290063T/C
2-4: 51358288T/G
2-4: 51402214T/G
2-4: 51424560G/A
2-4: 51447075G/A
2-4: 51473699G/A
2-4: 51517946G/A
2-4: 51605359G/A
2-4: 51620629T/C
2-4: 51646143T/C
2-4: 51692306G/C
2-4: 51707718A/G
2-4: 51747438C/G
2-4: 51875869A/G
2-4: 51879392A/T
2-4: 51905542T/C
2-4: 52060114G/A
2-4: 52063569C/T
2-4: 52141890T/C
2-4: 52154218T/C
2-4: 52167645A/T
2-4: 52185243G/T
2-4: 52221945A/T
2-4: 52267048T/G
2-4: 52268601G/A
2-4: 52335529A/C
2-4: 52390427A/G
2-4: 52448550G/A
2-4: 52448553A/G
2-4: 52449362G/A
2-4: 52486764A/C
2-4: 52512731C/A
2-4: 52514803G/T
2-4: 52543096A/G
2-4: 52581482A/G
2-4: 52593700C/A
2-4: 52621628G/A
2-4: 52663410G/A
2-4: 52663577G/A
2-4: 52682894T/A
2-4: 52694222G/A
2-4: 52711929G/C
2-4: 52723730A/G
2-4: 52735961G/A
2-4: 52751472C/T
2-4: 52765830C/T
2-4: 52785708C/A
2-4: 52837474A/G
2-4: 52837504G/A
2-4: 52862923C/T
2-4: 52864425A/C
2-4: 53008560T/A
2-4: 53114762C/T
2-4: 53169143T/C
2-4: 53208770A/G
2-4: 53294513C/G
2-4: 53296594C/T
2-4: 53326539C/T
2-4: 53347326A/T
2-4: 53382959C/T
2-4: 53411022A/G
2-4: 53447064G/C
2-4: 53485219A/G
2-4: 53518925G/A
2-4: 53535356A/G
2-4: 53570693C/T
2-4: 53584212C/T
2-4: 53604862C/A
2-4: 53696980A/G
2-4: 53725639C/T
2-4: 53778882A/G
2-4: 53791745C/T
2-4: 53820853C/G
2-4: 53932677T/C
2-4: 53988685A/G
2-4: 54023160T/C
2-4: 54057438T/C
2-4: 54079778A/G
2-4: 54092474T/C
2-4: 54123895A/T
2-4: 54201907C/T
2-4: 54239097A/G
2-4: 54365227T/C
2-4: 54391141T/C
2-4: 54394782A/T
2-4: 54417498A/C
2-4: 54455239G/T
2-4: 54479222A/G
2-4: 54493327T/A
2-4: 54540428A/C
2-4: 54597693G/A
2-4: 54631260T/A
2-4: 54662832C/T
2-4: 54697590A/G
2-4: 54731065C/T
2-4: 54785758A/T
2-4: 54851554G/C
2-4: 54962346T/A
2-4: 54975001T/C
2-4: 55033130A/C
2-4: 55048059C/A
2-4: 55127709G/C
2-4: 55176969A/T
2-4: 55220895C/A
2-4: 55284756T/A
2-4: 55341214C/T
2-4: 55396328G/A
2-4: 55467857G/A
2-4: 55518967G/A
2-4: 55569265A/G
2-4: 55609872C/T
2-4: 55610272C/T
2-4: 55623557C/T
2-4: 55628839C/T
2-4: 55637531A/G
2-4: 55644275G/C
2-4: 55644717A/T
2-4: 55714003G/A
2-4: 55744716A/G
2-4: 55792721A/G
2-4: 55804784T/C
2-4: 55893835C/A
2-4: 55898363C/T
2-4: 56040719A/T
2-4: 56046096T/A
2-4: 56057053A/G
2-4: 56092415A/T
2-4: 56165939A/G
2-4: 56203645T/A
2-4: 56305531A/G
2-4: 56340841T/A
2-4: 56414745G/A
2-4: 56629015T/C
2-4: 56666118A/G
2-4: 56711954G/C
2-4: 56793162T/C
2-4: 56833594A/G
2-4: 56846296A/T
2-4: 56858932G/A
2-4: 56890098T/A
2-4: 56916379A/G
2-4: 56918008A/C
2-4: 56977040A/T
2-4: 57020832T/C
2-4: 57073239G/A
2-4: 57170990G/C
2-4: 57174945C/A
2-4: 57181058G/A
2-4: 57246156G/A
2-4: 57279654C/G
2-4: 57313938C/T
2-4: 57343789A/G
2-4: 57373157C/G
2-4: 57489901A/C
2-4: 57500369C/A
2-4: 57532290C/T
2-4: 57550313T/G
2-4: 57656211G/A
2-4: 57658111G/T
2-4: 57701723C/T
2-4: 57748848T/C
2-4: 57765560G/C
2-4: 57813640T/A
2-4: 57916769G/A
2-4: 57949032C/T
2-4: 58007376C/T
2-4: 58055174C/T
2-4: 58134878C/.
2-4: 58139882G/A
2-4: 58155193G/C
2-4: 58287795T/C
2-4: 58298663T/C
2-4: 58311129T/A
2-4: 58325997C/A
2-4: 58428538T/C
2-4: 58430502T/G
2-4: 58430699T/G
2-4: 58431031T/C
2-4: 58431617T/C
2-4: 58433315G/A
2-4: 58433655T/G
2-4: 58435936A/G
2-4: 58455279A/T
2-4: 58487688C/T
2-4: 58574376A/G
2-4: 58590526G/A
2-4: 58610672C/G
2-4: 58625944C/T
2-4: 58722789A/G
2-4: 58775072T/C
2-4: 58802343T/A
2-4: 58812864G/A
2-4: 58846828C/A
2-4: 58889197G/A
2-4: 58934793T/C
2-4: 58965728T/C
2-4: 58996417T/G
2-4: 59057729C/T
2-4: 59099065T/A
2-4: 59110020C/T
2-4: 59133017G/T
2-4: 59143596A/C
2-4: 59158724G/A
2-4: 59190357A/G
2-4: 59227543C/T
2-4: 59276369T/C
2-4: 59358064C/G
2-4: 59458714C/A
2-4: 59505440C/T
2-4: 59609695A/T
2-4: 59613124G/T
2-4: 59623992A/G
2-4: 59708365T/C
2-4: 59711818A/G
2-4: 59809724A/G
2-4: 59898405G/A
2-4: 59957917A/G
2-4: 60044419G/A
2-4: 60091028A/C
2-4: 60129218T/A
2-4: 60145333A/T
2-4: 60210649A/G
2-4: 60237863T/A
2-4: 60302156T/A
2-4: 60395289A/C
2-4: 60432699A/G
2-4: 60447531G/T
2-4: 60489743T/G
2-4: 60580264G/A
2-4: 60641688C/T
2-4: 60670696A/T
2-4: 60738152G/A
2-4: 60964483T/C
2-4: 61017869C/T
2-4: 61019070T/A
2-4: 61019741C/A
2-4: 61020185A/G
2-4: 61020426C/T
2-4: 61020614C/T
2-4: 61021129G/A
2-4: 61199224G/A
2-4: 61330943A/T
2-4: 61352371A/C
2-4: 61438041T/C
2-4: 61574880C/A
2-4: 61598011C/T
2-4: 61620429G/A
2-4: 61653733A/G
2-4: 61756064T/C
2-4: 61827316T/C
2-4: 61884735A/T
2-4: 61930522A/G
2-4: 61959904T/C
2-4: 61973736A/G
2-4: 62025724G/T
2-4: 62078366C/T
2-4: 62205133T/G
2-4: 62265454C/T
2-4: 62276083A/T
2-4: 62313007C/T
2-4: 62381063G/A
2-4: 62383455C/T
2-4: 62383479A/G
2-4: 62384906A/G
2-4: 62402361T/A
2-4: 62445055C/A
2-4: 62463892A/G
2-4: 62499416G/A
2-4: 62546907G/C
2-4: 62769871T/C
2-4: 62779430G/T
2-4: 62796130C/G
2-4: 63007357T/C
2-4: 63119838T/C
2-4: 63134768G/C
2-4: 63185323G/T
2-4: 63310712G/A
2-4: 63328947A/T
2-4: 63339223G/T
2-4: 63360813G/C
2-4: 63361057G/T
2-4: 63375228T/C
2-4: 63414023C/A
2-4: 63484673T/G
2-4: 63520730G/A
2-4: 63594511T/C
2-4: 63665288A/G
2-4: 63702394C/A
2-4: 63714314A/C
2-4: 63715623A/C
2-4: 63800427C/T
2-4: 63906827T/A
2-4: 64045708C/T
2-4: 64164379T/G
2-4: 64171028T/C
2-4: 64533051A/T
2-4: 65079493A/G
2-4: 65107776C/A
3-1: 264675G/A
3-1: 362928C/A
3-1: 483690C/T
3-1: 898496T/C
3-1: 903400T/C
3-1: 910267C/T
3-1: 914619C/T
3-1: 944453C/T
3-1: 972040T/G
3-1: 1052224C/T
3-1: 1052876T/C
3-1: 1108242T/C
3-1: 1162341T/A
3-1: 1215227C/T
3-1: 1315796C/T
3-1: 1390288A/G
3-1: 1483719G/A
3-1: 1553631C/T
3-1: 1604295T/A
3-1: 1700006G/T
3-1: 1772279C/T
3-1: 1958314C/A
3-1: 2129247C/T
3-1: 2163225G/A
3-1: 2188878T/C
3-1: 2278522G/A
3-1: 2501707G/A
3-1: 2565770T/A
3-1: 2584382T/G
3-1: 2597138G/C
3-1: 2790702T/G
3-1: 2794659T/C
3-1: 2801443A/G
3-1: 2881889G/T
3-1: 3023505A/G
3-1: 3036464T/C
3-1: 3066978T/C
3-1: 3080388G/C
3-1: 3308751T/G
3-1: 3358930A/G
3-1: 3387902C/A
3-1: 3543588G/T
3-1: 3814336C/T
3-1: 4086565C/T
3-1: 4097384C/A
3-1: 4123243A/G
3-1: 4136455A/G
3-1: 4254668T/A
3-1: 4261986G/C
3-1: 4262510A/T
3-1: 4280962A/G
3-1: 4283200A/G
3-1: 4293753A/T
3-1: 4293894T/C
3-1: 4299939A/C
3-1: 4302180G/A
3-1: 4327815C/A
3-1: 4477761A/G
3-1: 4546922T/A
3-1: 4799146G/A
3-1: 5202126A/G
3-1: 5307293C/T
3-1: 5308355T/C
3-1: 5337870C/A
3-1: 5352505C/G
3-1: 5356228G/A
3-1: 5356256A/T
3-1: 5374461C/T
3-1: 5430933A/G
3-1: 5458265C/T
3-1: 5534621C/T
3-1: 5583454G/A
3-1: 5590928A/G
3-1: 5594454C/T
3-1: 5594844T/C
3-1: 5595218T/C
3-1: 5595220T/C
3-1: 5606105C/T
3-1: 5606517T/C
3-1: 5755824C/T
3-1: 5761656C/T
3-1: 5772148G/A
3-1: 5811802C/G
3-1: 5824181C/T
3-1: 5824518G/A
3-1: 5824544A/T
3-1: 5861886T/A
3-1: 5929712G/A
3-1: 5999297A/T
3-1: 6020103A/T
3-1: 6025026C/T
3-1: 6068259C/T
3-1: 6117052A/G
3-1: 6195341T/C
3-1: 6268703T/C
3-1: 6351607T/A
3-1: 6401210G/C
3-1: 6426271G/A
3-1: 6480792T/A
3-1: 6502706C/T
3-1: 6582735T/A
3-1: 6617522A/G
3-1: 6699114G/A
3-1: 6720443G/C
3-1: 6742849T/C
3-1: 6761749T/C
3-1: 6843247C/A
3-1: 6907187C/T
3-1: 7046068G/T
3-1: 7060526A/G
3-1: 7147067G/A
3-1: 7157694G/A
3-1: 7178168T/G
3-1: 7207038T/G
3-1: 7252936C/G
3-1: 7275263C/T
3-1: 7329264A/T
3-1: 7383937C/T
3-1: 7508995G/C
3-1: 7509023T/C
3-1: 7509030G/T
3-1: 7666985T/G
3-1: 7761140T/C
3-1: 7776410T/C
3-1: 7839614C/G
3-1: 7865856A/G
3-1: 7990712T/A
3-1: 8024117G/T
3-1: 8055629T/A
3-1: 8144458C/A
3-1: 8184097T/C
3-1: 8218722T/C
3-1: 8234203T/A
3-1: 8303472A/C
3-1: 8358922A/G
3-1: 8361637A/C
3-1: 8375379G/T
3-1: 8385765A/G
3-1: 8386200C/T
3-1: 8400151G/A
3-1: 8517409G/C
3-1: 8526710A/T
3-1: 8540273C/T
3-1: 8630690T/A
3-1: 8677456T/C
3-1: 8741871C/T
3-1: 8784123C/T
3-1: 8811240C/T
3-1: 8828891G/T
3-1: 8828899G/A
3-1: 8849997G/A
3-1: 8875925C/T
3-1: 8925079T/C
3-1: 9011031C/T
3-1: 9038479T/C
3-1: 9047074C/A
3-1: 9048715A/G
3-1: 9259459A/T
3-1: 9333504C/T
3-1: 9448102A/G
3-1: 9459395T/G
3-1: 9520240A/G
3-1: 9533645A/T
3-1: 9583923T/A
3-1: 9764754G/A
3-1: 9782356G/T
3-1: 9817980T/C
3-1: 9871779C/A
3-1: 9905463G/C
3-1: 9922722A/T
3-1: 9956748A/G
3-1: 9982928T/C
3-1: 10042544T/C
3-1: 10051056C/T
3-1: 10051834G/A
3-1: 10054888T/A
3-1: 10055600A/T
3-1: 10055973G/A
3-1: 10057880T/C
3-1: 10058027A/T
3-1: 10058138C/T
3-1: 10067645A/T
3-1: 10084714A/G
3-1: 10170542C/T
3-1: 10180968A/G
3-1: 10191169G/C
3-1: 10231473G/A
3-1: 10284739G/T
3-1: 10342366T/A
3-1: 10342612A/T
3-1: 10342674C/T
3-1: 10485690G/A
3-1: 10608795G/T
3-1: 10615300C/A
3-1: 10621342T/G
3-1: 10638973T/C
3-1: 10701310A/T
3-1: 10707126T/C
3-1: 10795517C/T
3-1: 10842971G/A
3-1: 10904395G/C
3-1: 10957418C/T
3-1: 10970908A/G
3-1: 10991801G/C
3-1: 10999617T/G
3-1: 11002158A/G
3-1: 11002303A/T
3-1: 11002859T/A
3-1: 11002891T/C
3-1: 11003364A/G
3-1: 11003406G/A
3-1: 11004428A/T
3-1: 11107131A/G
3-1: 11193506A/C
3-1: 11196700T/C
3-1: 11196775A/G
3-1: 11253175T/C
3-1: 11273301A/G
3-1: 11288091C/A
3-1: 11288237T/G
3-1: 11300393A/G
3-1: 11321724T/C
3-1: 11339341C/T
3-1: 11382892A/T
3-1: 11406718C/G
3-1: 11447305T/A
3-1: 11539201T/C
3-1: 11627865G/T
3-1: 11659752A/C
3-1: 11674408T/C
3-1: 11884553G/A
3-1: 11937534A/T
3-1: 12020385A/G
3-1: 12046795A/T
3-1: 12098182T/C
3-1: 12121397C/G
3-1: 12134557G/A
3-1: 12232011C/T
3-1: 12255913G/C
3-1: 12301644G/A
3-1: 12336599C/T
3-1: 12359469A/T
3-1: 12386161T/C
3-1: 12432389C/T
3-1: 12459082G/A
3-1: 12510733G/A
3-1: 12550551G/A
3-1: 12580881A/T
3-1: 12589048C/T
3-1: 12594699G/A
3-1: 12611141A/G
3-1: 12854915G/C
3-1: 12954612G/A
3-1: 12967090T/A
3-1: 13003958A/T
3-1: 13015915T/A
3-1: 13022943A/T
3-1: 13026034C/T
3-1: 13077436G/A
3-1: 13111124T/C
3-1: 13121244T/A
3-1: 13184708G/A
3-1: 13272124C/T
3-1: 13405634T/C
3-1: 13409149G/A
3-1: 13417336C/T
3-1: 13484518G/A
3-1: 13544810G/A
3-1: 13603625G/A
3-1: 13681656C/T
3-1: 13694785A/G
3-1: 13696454T/A
3-1: 13726793T/C
3-1: 13738448G/A
3-1: 13739409A/G
3-1: 13777591C/T
3-1: 13786348C/A
3-1: 13893544A/G
3-1: 13939958G/T
3-1: 13961995A/T
3-1: 13986204T/A
3-1: 14014986T/C
3-1: 14134770C/T
3-1: 14160003T/G
3-1: 14186133T/G
3-1: 14262251A/G
3-1: 14272727C/T
3-1: 14286891C/T
3-1: 14322681T/A
3-1: 14322991T/C
3-1: 14333844T/C
3-1: 14373426T/A
3-1: 14391785T/C
3-1: 14474510A/G
3-1: 14476877G/A
3-1: 14485042C/T
3-1: 14496866A/T
3-1: 14552489G/C
3-1: 14564203G/A
3-1: 14654338T/A
3-1: 14699828A/G
3-1: 14804891C/T
3-1: 14831393C/T
3-1: 14831827G/T
3-1: 14833149T/G
3-1: 14833919C/T
3-1: 14836448C/T
3-1: 14847215T/C
3-1: 14889578A/G
3-1: 14988606C/T
3-1: 15126794C/T
3-1: 15142321T/A
3-1: 15237177G/A
3-1: 15303780A/G
3-1: 15331675C/T
3-1: 15346963G/A
3-1: 15402408A/G
3-1: 15403719G/A
3-1: 15476285C/A
3-1: 15624419G/T
3-1: 15722082G/T
3-1: 15855075A/G
3-1: 15858246A/T
3-1: 15858313C/T
3-1: 15904885C/G
3-1: 15921237C/T
3-1: 15931682C/T
3-1: 15932077G/A
3-1: 15935288G/A
3-1: 15936046T/C
3-1: 15936255T/A
3-1: 15937116C/A
3-1: 15937338C/T
3-1: 15937564G/A
3-1: 15948026C/T
3-1: 15973030T/C
3-1: 15983066C/T
3-1: 16025710C/G
3-1: 16031484A/G
3-1: 16031532C/T
3-1: 16041491G/A
3-1: 16050535G/A
3-1: 16054870C/T
3-1: 16055240A/G
3-1: 16055345C/T
3-1: 16055365A/T
3-1: 16074822C/T
3-1: 16116352A/T
3-1: 16215573C/T
3-1: 16234280A/G
3-1: 16253264T/C
3-1: 16258180A/C
3-1: 16259739C/T
3-1: 16273881C/T
3-1: 16274180G/C
3-1: 16275951A/G
3-1: 16318459G/A
3-1: 16335955C/A
3-1: 16421996C/A
3-1: 16784941T/C
3-1: 16912221A/G
3-1: 16923109G/C
3-1: 16923768C/A
3-1: 16928049C/T
3-1: 16934654C/A
3-1: 16992248T/C
3-1: 17144650T/G
3-1: 17163531T/C
3-1: 17200591G/C
3-1: 17217792C/A
3-1: 17230837A/G
3-1: 17354796A/G
3-1: 17499900G/T
3-1: 17508618C/G
3-1: 17565942A/T
3-1: 17598628T/C
3-1: 17601408T/C
3-1: 17601616A/G
3-1: 17601691T/C
3-1: 17603551C/T
3-1: 17610196T/C
3-1: 17617062T/C
3-1: 17617543G/A
3-1: 17623221A/G
3-1: 17635199C/T
3-1: 17653791T/G
3-1: 17664654T/C
3-1: 17674991G/A
3-1: 17675610T/C
3-1: 17698550A/G
3-1: 17723791G/A
3-1: 17737936A/C
3-1: 17738537G/T
3-1: 17739092A/C
3-1: 17739111G/A
3-1: 17739500T/G
3-1: 17739894A/T
3-1: 17756102G/A
3-1: 17769727T/C
3-1: 17804827C/T
3-1: 17846024A/T
3-1: 17860873T/C
3-1: 17917929C/G
3-1: 17947932T/A
3-1: 17974724T/A
3-1: 17985802T/A
3-1: 18186963C/T
3-1: 18201392T/C
3-1: 18211613C/T
3-1: 18245977T/G
3-1: 18262544A/G
3-1: 18334693T/G
3-1: 18349170G/A
3-1: 18350330G/T
3-1: 18350941A/G
3-1: 18351257T/C
3-1: 18354390T/C
3-1: 18354735T/C
3-1: 18369755G/A
3-1: 18390704C/T
3-1: 18408238G/A
3-1: 18423421G/T
3-1: 18423819C/T
3-1: 18424067C/A
3-1: 18439706T/C
3-1: 18493815G/T
3-1: 18498451G/A
3-1: 18523732T/C
3-1: 18539577T/A
3-1: 18588770C/A
3-1: 18623211G/C
3-1: 18627429G/A
3-1: 18627486C/T
3-1: 18627709G/A
3-1: 18627825G/A
3-1: 18627937G/A
3-1: 18627954T/C
3-1: 18628088C/A
3-1: 18628210C/T
3-1: 18628786T/C
3-1: 18644961C/T
3-1: 18651664T/C
3-1: 18655872C/T
3-1: 18676026G/A
3-1: 18718993C/T
3-1: 18759066C/T
3-1: 18782069A/G
3-1: 18879610C/T
3-1: 18901714C/T
3-1: 18961987G/A
3-1: 19100746T/C
3-1: 19113581C/A
3-1: 19157667G/T
3-1: 19189037G/A
3-1: 19239012T/G
3-1: 19261806G/A
3-1: 19313498A/C
3-1: 19326642T/C
3-1: 19347951C/G
3-1: 19351937C/T
3-1: 19359865T/G
3-1: 19431030A/C
3-1: 19509523C/T
3-1: 19557345T/A
3-1: 19598623G/A
3-1: 19616234C/T
3-1: 19644623T/C
3-1: 19673416G/A
3-1: 19679183G/A
3-1: 19691141A/G
3-1: 19696293C/T
3-1: 19696831G/C
3-1: 19779966T/C
3-1: 19791997T/G
3-1: 19793599A/T
3-1: 19793613C/A
3-1: 19794128A/G
3-1: 19848963A/C
3-1: 19853369T/A
3-1: 19890211C/T
3-1: 19915832G/A
3-1: 19961260A/T
3-1: 19973173A/C
3-1: 19984260T/C
3-1: 19984413G/A
3-1: 19985346C/T
3-1: 19985554C/T
3-1: 19985699G/A
3-1: 19985721C/G
3-1: 19985977C/T
3-1: 19985981T/C
3-1: 19986127G/A
3-1: 19986820T/A
3-1: 19992685T/A
3-1: 20037614C/T
3-1: 20183171G/A
3-1: 20183720G/A
3-1: 20184124C/T
3-1: 20220379G/A
3-1: 20252018G/A
3-1: 20285230G/T
3-1: 20306287A/G
3-1: 20375349G/T
3-1: 20402619G/A
3-1: 20443776A/G
3-1: 20488435T/C
3-1: 20517862A/G
3-1: 20567058T/C
3-1: 20616593C/T
3-1: 20692582C/T
3-1: 20706407G/C
3-1: 20788746A/G
3-1: 20826237T/C
3-1: 20874904A/G
3-1: 21058997A/T
3-1: 21073919C/A
3-1: 21091689T/C
3-1: 21100471G/A
3-1: 21111477A/G
3-1: 21122815G/A
3-1: 21122819C/A
3-1: 21216248T/C
3-1: 21240459T/C
3-1: 21524820G/A
3-1: 21525946A/T
3-1: 21562925T/A
3-1: 21644291G/A
3-1: 21670928T/C
3-1: 21704919T/C
3-1: 21715358T/G
3-1: 21718139C/T
3-1: 21718626C/A
3-1: 21718773G/A
3-1: 21718892C/T
3-1: 21718902T/A
3-1: 21719058A/G
3-1: 21719457C/T
3-1: 21720143G/A
3-1: 21721147G/A
3-1: 21721247T/C
3-1: 21721267G/T
3-1: 21721551A/T
3-1: 21721597G/A
3-1: 21842324G/T
3-1: 21857541A/G
3-1: 21934282A/G
3-1: 21999250G/T
3-1: 22004723C/G
3-1: 22039010T/G
3-1: 22090332G/A
3-1: 22134460G/A
3-1: 22148395A/G
3-1: 22186617C/T
3-1: 22227878A/G
3-1: 22246541G/A
3-1: 22370930G/A
3-1: 22469954T/C
3-1: 22480430A/G
3-1: 22504779C/T
3-1: 22514252G/A
3-1: 22526189C/T
3-1: 22533669T/C
3-1: 22536482C/T
3-1: 22564439C/T
3-1: 22597675C/T
3-1: 22628063A/G
3-1: 22629233C/T
3-1: 22668653T/A
3-1: 22697934A/T
3-1: 22797686G/A
3-1: 22816781C/A
3-1: 22860711T/C
3-1: 22898646A/G
3-1: 22939248C/A
3-1: 22983291C/T
3-1: 23016270G/T
3-1: 23041078T/C
3-1: 23182161G/A
3-1: 23184469G/C
3-1: 23278710G/A
3-1: 23348952T/C
3-1: 23403753G/C
3-1: 23442204A/G
3-1: 23488801G/C
3-1: 23527007T/C
3-1: 23529240A/G
3-1: 23554611G/A
3-1: 23609388T/A
3-1: 23617482T/G
3-1: 23621903C/T
3-1: 23627898G/C
3-1: 23631771A/G
3-1: 23631782C/T
3-1: 23659194C/T
3-1: 23688039C/A
3-1: 23688156G/T
3-1: 23688482T/C
3-1: 23701704C/T
3-1: 23739973A/C
3-1: 23762122C/T
3-1: 23787306G/A
3-1: 23865424A/C
3-1: 23891832C/A
3-1: 23913889G/A
3-1: 23947279T/A
3-1: 24016738T/C
3-1: 24064648C/A
3-1: 24080366T/C
3-1: 24111930A/G
3-1: 24133455C/G
3-1: 24150329T/G
3-1: 24193764C/T
3-1: 24209676C/T
3-1: 24223992T/C
3-1: 24234411A/G
3-1: 24255421G/A
3-1: 24279163C/G
3-1: 24290933G/T
3-1: 24337393T/C
3-1: 24353706A/G
3-1: 24374122C/T
3-1: 24400175G/A
3-1: 24518549C/T
3-1: 24550821C/T
3-1: 24576604C/T
3-1: 24654316T/A
3-1: 24702010T/A
3-1: 24734057C/A
3-1: 24791995A/G
3-1: 24880473T/C
3-1: 24908684T/A
3-1: 25003213C/T
3-1: 25042814T/A
3-1: 25095041T/C
3-1: 25223654G/A
3-1: 25237191A/C
3-1: 25361504T/C
3-1: 25372225T/C
3-1: 25423343T/G
3-1: 25540128G/A
3-1: 25547538A/T
3-1: 25548698T/C
3-1: 25592865G/T
3-1: 25670108G/A
3-1: 25716019C/T
3-1: 25745133T/G
3-1: 25785908G/A
3-1: 25803495G/T
3-1: 25804947T/C
3-1: 25804958A/G
3-1: 26033759A/G
3-1: 26035503G/T
3-1: 26084477G/T
3-1: 26101745A/G
3-1: 26110237C/A
3-1: 26166959A/G
3-1: 26199966T/C
3-1: 26362654T/C
3-1: 26384194C/A
3-1: 26394725A/T
3-1: 26398759G/T
3-1: 26420830G/T
3-1: 26477060A/C
3-1: 26515268T/C
3-1: 26549061G/A
3-1: 26549334G/A
3-1: 26648545G/A
3-1: 26672861A/G
3-1: 26698450G/A
3-1: 26734922T/A
3-1: 26755583G/A
3-1: 26809559G/T
3-1: 26827929C/T
3-1: 26928760T/A
3-1: 26953649G/A
3-1: 26969465G/C
3-1: 26988274G/T
3-1: 27056927T/C
3-1: 27089669T/C
3-1: 27200521C/G
3-1: 27277673A/T
3-1: 27397658T/G
3-1: 27430267T/G
3-1: 27608673A/T
3-1: 27642330T/C
3-1: 27789409T/C
3-1: 27791470C/A
3-1: 27805323G/A
3-1: 27870676A/G
3-1: 27995085T/G
3-1: 28041263T/C
3-1: 28073429G/C
3-1: 28142626T/C
3-1: 28228456A/G
3-1: 28228910G/A
3-1: 28229016A/G
3-1: 28229809T/C
3-1: 28229920T/C
3-1: 28230312C/T
3-1: 28230435C/T
3-1: 28231284C/T
3-1: 28402359C/T
3-1: 28459652C/T
3-1: 28473928G/A
3-1: 28518138G/A
3-1: 28532495A/C
3-1: 28542755A/T
3-1: 28600034A/G
3-1: 28778454A/C
3-1: 28822966T/C
3-1: 29158544C/G
3-1: 29168473C/G
3-1: 29186050T/C
3-1: 29261072G/A
3-1: 29402395T/C
3-1: 29519517G/A
3-1: 29584514T/C
3-1: 29710318G/A
3-1: 29811043G/T
3-1: 29982334A/C
3-1: 29984284A/G
3-1: 30018211C/T
3-1: 30091211G/T
3-1: 30111677G/T
3-1: 30116508C/T
3-1: 30118031G/C
3-1: 30118226A/G
3-1: 30123875A/G
3-1: 30124020A/T
3-1: 30124314T/G
3-1: 30125634T/C
3-1: 30128240C/T
3-1: 30143079A/G
3-1: 30150867G/T
3-1: 30150913T/A
3-1: 30151340A/C
3-1: 30154018T/G
3-1: 30167163G/C
3-1: 30273621T/C
3-1: 30284707C/A
3-1: 30286330A/T
3-1: 30286345A/T
3-1: 30294926A/T
3-1: 30343630A/T
3-1: 30360119C/T
3-1: 30397710G/A
3-1: 30464851T/C
3-1: 30465100T/C
3-1: 30465204G/T
3-1: 30465336C/A
3-1: 30465529A/G
3-1: 30470576G/A
3-1: 30478366A/G
3-1: 30514490G/C
3-1: 30549254A/T
3-1: 30636125A/G
3-1: 30719960T/C
3-1: 30724280G/A
3-1: 30903875T/A
3-1: 30964436T/C
3-1: 31009527T/C
3-1: 31060515G/A
3-1: 31071811G/A
3-1: 31127604C/A
3-1: 31306809C/T
3-1: 31414427G/A
3-1: 31622660C/T
3-1: 31635214C/A
3-1: 31650190T/G
3-1: 31652307T/C
3-1: 31652310G/A
3-1: 31868825C/T
3-1: 32002692G/T
3-1: 32063086G/A
3-1: 32094897A/T
3-1: 32123421G/A
3-1: 32135721C/T
3-1: 32660126A/C
3-1: 32709106T/A
3-1: 32863815C/T
3-1: 32967851A/T
3-1: 33190698A/T
3-1: 33227338T/A
3-1: 33315089A/C
3-1: 33515293G/A
3-1: 33860735G/T
3-1: 33902809C/G
3-1: 33918440T/C
3-1: 34139578G/A
3-1: 34139645A/G
3-1: 34200208G/A
3-1: 34228508T/C
3-1: 34286864C/T
3-1: 34512448A/G
3-1: 34671277C/A
3-1: 34863861T/C
3-1: 34939243A/T
3-1: 35205250C/T
3-1: 35404119A/T
3-1: 35494921A/C
3-1: 35534328T/A
3-1: 35718408G/T
3-1: 35729156T/A
3-1: 35731871G/A
3-1: 35894031T/C
3-1: 35927249T/A
3-1: 35961759G/A
3-1: 35973950A/T
3-1: 35997548G/A
3-1: 36169388G/A
3-1: 36198344G/A
3-1: 36210951C/T
3-1: 36246130C/T
3-1: 36387047T/G
3-1: 36395663G/T
3-1: 36395899C/A
3-1: 36398792C/A
3-1: 36430144A/T
3-1: 36441958G/A
3-1: 36452200C/T
3-1: 36625943G/A
3-1: 36727382A/G
3-1: 36755977T/A
3-1: 36783208A/T
3-1: 36955803A/G
3-1: 37070734G/A
3-1: 37132407A/T
3-1: 37191200T/C
3-1: 37243049A/G
3-1: 37269677G/A
3-1: 37285575T/C
3-1: 37302953T/C
3-1: 37313015C/T
3-1: 37323621A/G
3-1: 37324156G/C
3-1: 37334711C/T
3-1: 37354795G/A
3-1: 37401510A/T
3-1: 37460570T/C
3-1: 37531861G/T
3-1: 37532766A/G
3-1: 37595202T/C
3-1: 37670753A/C
3-1: 37791516C/T
3-1: 37818018G/A
3-1: 37885055G/A
3-1: 38009396T/G
3-1: 38331375T/C
3-1: 38523319C/A
3-1: 38597626C/A
3-1: 38914855T/A
3-1: 38968367C/T
3-1: 39002221A/G
3-1: 39143007C/T
3-1: 39195232G/T
3-1: 39222910G/A
3-1: 39305621C/A
3-1: 39356475G/A
3-1: 39408365G/A
3-1: 39422027C/T
3-1: 39426947A/T
3-1: 39449675T/C
3-1: 39463666C/T
3-1: 39518827A/G
3-1: 39680399T/G
3-1: 39710836G/A
3-1: 39836157G/A
3-1: 39867407C/T
3-1: 39899359A/C
3-1: 40082769C/T
3-1: 40185098A/T
3-1: 40250451T/A
3-1: 40408255G/A
3-1: 40435610A/G
3-1: 40448416G/A
3-1: 40520427A/G
3-1: 40749140T/G
3-1: 40756362C/T
3-1: 40756363C/T
3-1: 40756405T/A
3-1: 40756454G/A
3-1: 40756465G/A
3-1: 40756524C/T
3-1: 40757331C/A
3-1: 40757351A/G
3-1: 40757391C/.
3-1: 40757450A/T
3-1: 40757524C/T
3-1: 40757879T/A
3-1: 40763286T/C
3-1: 40896783G/T
3-1: 41053318G/A
3-1: 41140961C/T
3-1: 41193018C/T
3-1: 41280977C/T
3-1: 41927561G/A
3-1: 41989655C/T
3-1: 42007564A/T
3-1: 42519362A/C
3-1: 42572982C/T
3-1: 42596199T/C
3-1: 42676989T/C
3-1: 42715768G/A
3-1: 42727504C/T
3-1: 42768054C/T
3-1: 42796577G/A
3-1: 42994593C/G
3-1: 43009163G/A
3-1: 43034725A/C
3-1: 43076677T/C
3-1: 43122198G/T
3-1: 43236622C/T
3-1: 43241659G/A
3-1: 43317811T/C
3-1: 43357102T/C
3-1: 43358214G/A
3-1: 43411695A/T
3-1: 43461158A/T
3-1: 43801519C/G
3-1: 43943879A/G
3-1: 44004009A/T
3-1: 44033154T/A
3-1: 44093525G/A
3-1: 44231029T/C
3-1: 44235157T/A
3-1: 44241157A/T
3-1: 44260586G/A
3-1: 44321024T/A
3-1: 44386913C/T
3-1: 44508855T/G
3-1: 44511411C/T
3-1: 44559525G/A
3-1: 44613219G/T
3-1: 44646065A/T
3-1: 44708303C/T
3-1: 44809202C/T
3-1: 44846349A/G
3-1: 44949277C/T
3-1: 45062519A/G
3-1: 45090994G/A
3-1: 45113200A/G
3-1: 45306612G/A
3-1: 45344918T/A
3-1: 45472320C/A
3-1: 45508243G/A
3-1: 45555705C/T
3-1: 45601778C/T
3-1: 45690686G/C
3-1: 45736941G/A
3-1: 45830977C/T
3-1: 45877233G/A
3-1: 45921485G/T
3-1: 45947757T/A
3-1: 45973137T/A
3-1: 45996842T/A
3-1: 46010843T/C
3-1: 46061725C/T
3-1: 46146085G/A
3-1: 46179912A/G
3-1: 46229739A/T
3-1: 46279203A/G
3-1: 46322209T/C
3-1: 46377765C/T
3-1: 46390740C/T
3-1: 46401666G/A
3-1: 46431093C/T
3-1: 46470598A/T
3-1: 46472211G/A
3-1: 46570889C/T
3-1: 46571078C/T
3-1: 46584116T/C
3-1: 46598480A/G
3-1: 46754036G/A
3-1: 46755154C/T
3-1: 46838599G/C
3-1: 46852742G/T
3-1: 46881650C/G
3-1: 46913996T/C
3-1: 46956429T/C
3-1: 46994114T/A
3-1: 47071767A/G
3-1: 47071796A/C
3-1: 47071973G/T
3-1: 47083652A/G
3-1: 47177938T/C
3-1: 47193457A/T
3-1: 47195420A/C
3-1: 47251807G/A
3-1: 47307704A/T
3-1: 47373612A/C
3-1: 47384848A/G
3-1: 47446973A/C
3-1: 47523933T/C
3-1: 47582673A/C
3-1: 47585695T/A
3-1: 47587372T/G
3-1: 47615487A/G
3-1: 47685131C/T
3-1: 47832871A/T
3-1: 47985436G/T
3-1: 48072122G/A
3-1: 48108190A/G
3-1: 48189416A/T
3-1: 48328960T/A
3-1: 48402710A/C
3-1: 48700143C/G
3-1: 48856735G/A
3-1: 48882455A/G
3-1: 48971345T/A
3-1: 49030013G/C
3-1: 49189083C/T
3-1: 49246241G/A
3-1: 49277530G/A
3-1: 49309511G/C
3-1: 49324170C/A
3-1: 49346266T/C
3-1: 49407582C/A
3-1: 49446373C/G
3-1: 49502880A/T
3-1: 49523661G/A
3-1: 49549282A/G
3-1: 49613341A/G
3-1: 49647900G/A
3-1: 49677152A/G
3-1: 49705179A/T
3-1: 49761465T/C
3-1: 49860730A/G
3-1: 49898430G/A
3-1: 49913989A/C
3-1: 49976934A/T
3-1: 50019713G/A
3-1: 50092108G/A
3-1: 50264871C/T
3-1: 50367968G/A
3-1: 50408866G/A
3-1: 50519466A/T
3-1: 50519870T/C
3-1: 50521118A/G
3-1: 50621302A/G
3-1: 50641107G/A
3-1: 50653573A/G
3-1: 50729080G/A
3-1: 50792946G/A
3-1: 50852254T/.
3-1: 50852349G/.
3-1: 50870445A/C
3-1: 50932618T/G
3-1: 50981344A/G
3-1: 50997786C/T
3-1: 51045337G/A
3-1: 51059304T/C
3-1: 51070316T/C
3-1: 51096256A/G
3-1: 51168337G/A
3-1: 51192959G/A
3-1: 51203815A/C
3-1: 51214456C/A
3-1: 51251569C/T
3-1: 51294681G/T
3-1: 51338689T/C
3-1: 51338978A/T
3-1: 51349747T/G
3-1: 51365564A/G
3-1: 51440536T/A
3-1: 51459755C/G
3-1: 51460261T/C
3-1: 51460283T/C
3-1: 51548760C/G
3-1: 51605202G/C
3-1: 51623425C/T
3-1: 51665819G/A
3-1: 51697934T/C
3-1: 51774336C/T
3-1: 51776072T/C
3-1: 51791267A/G
3-1: 51809000A/G
3-1: 51816651G/T
3-1: 51816956T/A
3-1: 51818309C/A
3-1: 51828271G/A
3-1: 51882679G/A
3-1: 51924857C/T
3-1: 51944489T/G
3-1: 51994088C/T
3-1: 52048313C/T
3-1: 52082365A/T
3-1: 52107854G/A
3-1: 52140046C/G
3-1: 52155640C/T
3-1: 52238633C/T
3-1: 52341303G/A
3-1: 52374167C/T
3-1: 52451254C/T
3-1: 52465913T/G
3-1: 52511493C/T
3-1: 52517417C/A
3-1: 52518387A/C
3-1: 52518854C/T
3-1: 52518872T/C
3-1: 52519010T/A
3-1: 52519416T/C
3-1: 52527854A/T
3-1: 52528152C/T
3-1: 52544384G/A
3-1: 52557275G/A
3-1: 52567591T/C
3-1: 52585237T/C
3-1: 52644692C/T
3-1: 52711444T/C
3-1: 52755560A/G
3-1: 52763283T/A
3-1: 52873358C/T
3-1: 52905855A/G
3-1: 53000272T/C
3-1: 53001150C/T
3-1: 53001265A/C
3-1: 53020582A/T
3-1: 53045439G/A
3-1: 53070956C/T
3-1: 53101221A/G
3-1: 53161799C/T
3-1: 53299742T/C
3-1: 53360553G/A
3-1: 53383821T/C
3-1: 53394260T/C
3-1: 53394654A/G
3-1: 53394662T/C
3-1: 53395287G/T
3-1: 53405836A/G
3-1: 53551768A/C
3-1: 53578291G/T
3-1: 53592528C/G
3-1: 53641889T/A
3-1: 53704317A/G
3-1: 53721419G/A
3-1: 53747196T/A
3-1: 53820195G/A
3-1: 53852914C/T
3-1: 53879574A/G
3-1: 53890221G/A
3-1: 53957662A/T
3-1: 53984957G/A
3-1: 54037756G/C
3-1: 54053246T/C
3-1: 54081748C/G
3-1: 54094219A/G
3-1: 54106308C/T
3-1: 54127703C/T
3-1: 54146204C/T
3-1: 54147695G/T
3-1: 54175254T/C
3-1: 54221963T/A
3-1: 54254880C/G
3-1: 54328753A/C
3-1: 54427198C/T
3-1: 54523540T/G
3-1: 54537127T/C
3-1: 54537148G/A
3-1: 54537161G/A
3-1: 54601140A/T
3-1: 54715845T/C
3-1: 54717685C/G
3-1: 54729217C/T
3-1: 54838951G/T
3-1: 54843252C/T
3-1: 54983044T/C
3-1: 55082076G/A
3-1: 55103981C/T
3-1: 55184077A/G
3-1: 55198077T/A
3-1: 55230584G/C
3-1: 55232114T/G
3-1: 55232216A/G
3-1: 55232306A/G
3-1: 55236358T/C
3-1: 55239037C/T
3-1: 55239259T/A
3-1: 55246734T/A
3-1: 55329625C/T
3-1: 55364805A/T
3-1: 55450076A/G
3-1: 55468084A/G
3-1: 55490045A/G
3-1: 55510500T/C
3-1: 55581579A/G
3-1: 55676947C/T
3-1: 55766905C/T
3-1: 55794430G/A
3-1: 55851210T/A
3-1: 55936851G/A
3-1: 55953946T/.
3-1: 55957589G/T
3-1: 55984484A/G
3-1: 55985405G/A
3-1: 55985520A/G
3-1: 55999029C/A
3-1: 56010374T/C
3-1: 56018563C/T
3-1: 56019241G/T
3-1: 56019343C/T
3-1: 56019581A/C
3-1: 56020009T/C
3-1: 56020560A/G
3-1: 56041624T/A
3-1: 56057826G/C
3-1: 56070021A/G
3-1: 56076268G/A
3-1: 56076448G/T
3-1: 56076759G/A
3-1: 56076992A/C
3-1: 56077368G/A
3-1: 56078708T/G
3-1: 56078839A/G
3-1: 56079033T/C
3-1: 56079192T/C
3-1: 56079400C/G
3-1: 56079641A/G
3-1: 56079756C/A
3-1: 56079857T/G
3-1: 56080115T/C
3-1: 56080716T/G
3-1: 56080765C/A
3-1: 56081230T/C
3-1: 56081674G/A
3-1: 56110314T/G
3-1: 56120632G/A
3-1: 56132604G/A
3-1: 56136400G/T
3-1: 56172260G/A
3-1: 56183185T/A
3-1: 56183672G/A
3-1: 56185199G/A
3-1: 56194224G/A
3-1: 56200259A/G
3-1: 56204554G/A
3-1: 56207494G/T
3-1: 56208026T/A
3-1: 56254321T/A
3-1: 56297297A/C
3-1: 56306167A/G
3-1: 56307929G/A
3-1: 56319730C/T
3-1: 56321453C/T
3-1: 56321766G/A
3-1: 56327554C/T
3-1: 56327633C/T
3-1: 56327935G/T
3-1: 56333031A/G
3-1: 56346927C/T
3-1: 56361038T/A
3-1: 56368243T/A
3-1: 56375200C/T
3-1: 56379720T/C
3-1: 56391349C/T
3-1: 56438541G/A
3-1: 56471030G/A
3-1: 56493108T/G
3-1: 56526362T/G
3-1: 56580665C/T
3-1: 56611032A/G
3-1: 56665830G/A
3-1: 56744073C/T
3-1: 56754110G/A
3-1: 56793105A/T
3-1: 56799744C/A
3-1: 56867642T/C
3-1: 56902711G/A
3-1: 56933971G/A
3-1: 56967142A/G
3-1: 56999169G/C
3-1: 57130734T/A
3-1: 57360186C/T
3-1: 57476415A/G
3-1: 57543235G/T
3-1: 57675342C/T
3-1: 57696783A/G
3-1: 57708644A/G
3-1: 57760449G/A
3-1: 57977901C/T
3-1: 58057853G/T
3-1: 58097951A/C
3-1: 58124936A/G
3-1: 58125083C/T
3-1: 58138269A/G
3-1: 58157320A/T
3-1: 58197235C/T
3-1: 58237690T/G
3-1: 58305090G/A
3-1: 58318668G/C
3-1: 58352909G/C
3-1: 58387171T/C
3-1: 58413677C/T
3-1: 58463047G/T
3-1: 58472169G/A
3-1: 58481181G/A
3-1: 58491264C/T
3-1: 58513775C/A
3-1: 58514950C/G
3-1: 58552788C/T
3-1: 58616367C/A
3-1: 58725791A/C
3-1: 58746292C/T
3-1: 58757188G/A
3-1: 58804716T/C
3-1: 58886602G/A
3-1: 58948172C/A
3-1: 58984388A/G
3-1: 58988654C/G
3-1: 58989471C/A
3-1: 59004118A/G
3-1: 59018106C/G
3-1: 59041999A/G
3-1: 59067742G/A
3-1: 59218632G/A
3-1: 59220950T/C
3-1: 59265397C/T
3-1: 59303372C/T
3-1: 59389661A/T
3-1: 59390597C/T
3-1: 59392324C/T
3-1: 59414810T/C
3-1: 59455009C/G
3-1: 59541243T/C
3-1: 59554587T/G
3-1: 59567381G/A
3-1: 59578163C/T
3-1: 59617346G/A
3-1: 59674560A/G
3-1: 59720458A/G
3-1: 59750516C/A
3-1: 59771219G/A
3-1: 59790123T/A
3-1: 59811949G/T
3-1: 59824957C/A
3-1: 59839393G/A
3-1: 59888105A/G
3-1: 59905579C/T
3-1: 59918289C/T
3-1: 59918469T/C
3-1: 59927075A/T
3-1: 59927485A/G
3-1: 59929272T/G
3-1: 59930462A/G
3-1: 59937351G/T
3-1: 59937464A/G
3-1: 59937622A/G
3-1: 59937749T/A
3-1: 59937941G/A
3-1: 59940766A/G
3-1: 59985291T/A
3-1: 60000015C/G
3-1: 60032712C/T
3-1: 60041695T/C
3-1: 60050186C/T
3-1: 60062462A/G
3-1: 60081961C/T
3-1: 60084702C/T
3-1: 60084835G/T
3-1: 60089758G/A
3-1: 60089940T/C
3-1: 60090080G/A
3-1: 60090403C/T
3-1: 60090414T/A
3-1: 60090516A/T
3-1: 60090526G/T
3-1: 60090822G/A
3-1: 60090956A/T
3-1: 60092679T/G
3-1: 60094391G/T
3-1: 60095200C/T
3-1: 60095704G/A
3-1: 60129285C/A
3-1: 60189207G/T
3-1: 60204868A/G
3-1: 60228620G/A
3-1: 60244812G/A
3-1: 60294190T/C
3-1: 60308571C/T
3-1: 60365561C/T
3-1: 60366743T/G
3-1: 60423506G/C
3-1: 60483229G/A
3-1: 60534001C/T
3-1: 60645225T/A
3-1: 60720555T/C
3-1: 60720635T/C
3-1: 60720780C/G
3-1: 60721123G/A
3-1: 60721162A/G
3-1: 60734283A/T
3-1: 60734819A/G
3-1: 60746785T/A
3-1: 60750109G/A
3-1: 60774435G/A
3-1: 60784966T/A
3-1: 60785745C/T
3-1: 60796280G/C
3-1: 60798047G/A
3-1: 60804798G/C
3-1: 60804872G/C
3-1: 60806907G/A
3-1: 60904320G/A
3-1: 60931368T/C
3-1: 60952858G/A
3-1: 60963577G/A
3-1: 60966836A/G
3-1: 61020986C/T
3-1: 61140106T/A
3-1: 61166625T/C
3-1: 61188947G/A
3-1: 61256013G/A
3-1: 61297016C/T
3-1: 61327367T/G
3-1: 61338561A/G
3-1: 61355417T/A
3-1: 61412064G/A
3-1: 61470690G/C
3-1: 61529731C/T
3-1: 61572493C/G
3-1: 61622039T/A
3-1: 61650286A/T
3-1: 61711504A/G
3-1: 61776766T/C
3-1: 61828436C/T
3-1: 61859796G/A
3-1: 61870405A/G
3-1: 61881851A/G
3-1: 61937310G/A
3-1: 62017121C/T
3-1: 62093314T/C
3-1: 62117824A/G
3-1: 62385934A/C
3-1: 62502230G/A
3-1: 62582719G/C
3-1: 62593076C/T
3-1: 62608677C/T
3-1: 62662202A/T
3-1: 62666576A/G
3-1: 62713075A/T
3-1: 62752836T/C
3-1: 62754357A/C
3-1: 62778803G/C
3-1: 62825625T/C
3-1: 62828226C/T
3-1: 62853133T/C
3-1: 62867002C/T
3-1: 62996591G/A
3-1: 63037839C/T
3-1: 63037972G/A
3-1: 63040975G/C
3-1: 63044131G/A
3-1: 63045723G/A
3-1: 63056337A/C
3-1: 63064062G/A
3-1: 63064221G/A
3-1: 63083770G/A
3-1: 63098688T/G
3-1: 63132500A/G
3-1: 63182946A/T
3-1: 63193627A/T
3-1: 63208373C/T
3-1: 63219494G/C
3-1: 63234510A/G
3-1: 63280824G/A
3-1: 63309311T/A
3-1: 63338707C/T
3-1: 63377190T/A
3-1: 63403768A/T
3-1: 63507083C/T
3-1: 63519532A/C
3-1: 63531066A/T
3-1: 63624972C/A
3-1: 63645487T/C
3-1: 63665837G/A
3-1: 63708671C/G
3-1: 63743040A/T
3-1: 63743376G/A
3-1: 63743611G/C
3-1: 63754011A/G
3-1: 63766311T/G
3-1: 63795584T/C
3-1: 63834405T/A
3-1: 63873380A/T
3-1: 63899065A/T
3-1: 63950545A/G
3-1: 63975075G/T
3-1: 64001882C/T
3-1: 64043654G/A
3-1: 64082770T/A
3-1: 64137976T/A
3-1: 64190372G/T
3-1: 64215693C/T
3-1: 64249218C/T
3-1: 64279274G/T
3-1: 64297152G/T
3-1: 64322187T/G
3-1: 64356135T/C
3-1: 64398738C/T
3-1: 64409145A/G
3-1: 64419845A/T
3-1: 64480763T/C
3-1: 64628672T/C
3-1: 64747982G/T
3-1: 64847822A/C
3-1: 64864234C/A
3-1: 64864351G/C
3-1: 64893211C/T
3-1: 64908790C/G
3-1: 65081727G/A
3-1: 65120014C/T
3-1: 65165600A/T
3-1: 65176555T/A
3-1: 65277633C/T
3-1: 65332474C/T
3-1: 65372349C/T
3-1: 65401098A/C
3-1: 65448396G/A
3-1: 65474211A/G
3-1: 65536378T/C
3-1: 65569787G/A
3-1: 65666847A/G
3-1: 65708536A/G
3-1: 65722816G/C
3-1: 65735925C/T
3-1: 65769100A/T
3-1: 65807080T/C
3-1: 65827907T/G
3-1: 65882381T/G
3-1: 65952019G/A
3-1: 65996371G/A
3-1: 66032284C/T
3-1: 66112019T/C
3-1: 66149794C/A
3-1: 66197188T/C
3-1: 66236213T/C
3-1: 66273646G/C
3-1: 66280517G/A
3-1: 66302832T/C
3-1: 66347251A/G
3-1: 66347568T/C
3-1: 66369314C/T
3-1: 66395058A/G
3-1: 66540962T/G
3-1: 66575506G/T
3-1: 66585573C/T
3-1: 66597893T/C
3-1: 66617788A/G
3-1: 66654306A/G
3-1: 66675448A/G
3-1: 66713530G/A
3-1: 66732843G/A
3-1: 66740413C/T
3-1: 66812889G/A
3-1: 66844597T/C
3-1: 66922861T/C
3-1: 66980711A/T
3-1: 67005166A/T
3-1: 67007058T/A
3-1: 67019190T/A
3-1: 67087633A/G
3-1: 67120015G/A
3-1: 67152760G/A
3-1: 67187202A/T
3-1: 67248660C/A
3-1: 67277065A/T
3-1: 67323493G/A
3-1: 67349671G/T
3-1: 67350555A/G
3-1: 67378281G/A
3-1: 67403980T/C
3-1: 67447059A/C
3-1: 67507875G/C
3-1: 67577759A/G
3-1: 67627633G/A
3-1: 67630215G/A
3-1: 67679399T/C
3-1: 67719019C/T
3-1: 67757286C/A
3-1: 67825347G/A
3-1: 67853036G/T
3-1: 67991654T/C
3-1: 68113447G/A
3-1: 68193411C/A
3-1: 68281688T/C
3-1: 68312749C/T
3-1: 68382407G/A
3-1: 68452955T/C
3-1: 68492367T/C
3-1: 68528224C/T
3-1: 68589841C/T
3-1: 68632694G/A
3-1: 68666839G/A
3-1: 68715879T/C
3-1: 68736906C/T
3-1: 68828722T/G
3-1: 68870761T/G
3-1: 68916187C/T
3-1: 68992350C/T
3-1: 69036053C/A
3-1: 69055148G/A
3-1: 69224479A/C
3-1: 69355561T/G
3-1: 69357263G/A
3-1: 69406319A/C
3-1: 69497905C/T
3-1: 69526925A/G
3-1: 69571297T/C
3-1: 69630672C/A
3-1: 69669598T/A
3-1: 69828721T/C
3-1: 69914096A/C
3-1: 70008657G/A
3-1: 70044412T/C
3-1: 70125440G/C
3-1: 70177843G/T
3-1: 70340006C/T
3-1: 70362489A/G
3-1: 70464801C/G
3-1: 70518875A/G
3-1: 70568868A/G
3-1: 70569608A/G
3-1: 70579352G/A
3-1: 70580306A/T
3-1: 70580732C/G
3-1: 70580926A/G
3-1: 70581295C/A
3-1: 70582673G/A
3-1: 70583307G/A
3-1: 70591815T/C
3-1: 70592165G/A
3-1: 70592703G/C
3-1: 70620738C/A
3-1: 70738412T/A
3-1: 70747345T/A
3-1: 70758497C/T
3-1: 70814192G/A
3-1: 70844107G/A
3-1: 70958454T/C
3-1: 71055632T/C
3-1: 71096526T/G
3-1: 71118442G/T
3-1: 71148201C/T
3-1: 71176973A/G
3-1: 71212722T/A
3-1: 71222786T/C
3-1: 71224569T/C
3-1: 71292809G/T
3-1: 71377881T/C
3-1: 71397158T/C
3-1: 71406490T/G
3-1: 71410072G/T
3-1: 71450944A/T
3-1: 71476589G/A
3-1: 71478492A/G
3-1: 71478934T/A
3-1: 71652454T/A
3-1: 71654350A/C
3-1: 71709003G/A
3-1: 71784529T/C
3-1: 71802453A/T
3-1: 71887893G/A
3-1: 71895841G/A
3-1: 71944409G/A
3-1: 71989345A/G
3-1: 72183597C/A
3-1: 72288815C/T
3-1: 72607935G/A
3-1: 72662489G/A
3-1: 72674064A/G
3-1: 72784153A/G
3-1: 72810823A/T
3-1: 72841852T/A
3-1: 72865376G/A
3-1: 72888869A/G
3-1: 72937038G/A
3-1: 72949429A/G
3-1: 72988679T/A
3-1: 73022158G/T
3-1: 73033196G/C
3-1: 73063356G/A
3-1: 73063392G/C
3-1: 73090009A/G
3-1: 73212314T/C
3-1: 73303288A/G
3-1: 73372238G/A
3-1: 73436926A/T
3-1: 73482663C/T
3-1: 73540552G/T
3-1: 73638265A/G
3-1: 73745283A/G
3-1: 73982339A/G
3-1: 74146357T/G
3-1: 74246879A/G
3-1: 74282244G/A
3-1: 74335593T/G
3-1: 74408437G/A
3-1: 74417101G/A
3-1: 74420699G/A
3-1: 74454446T/C
3-1: 74564943T/C
3-1: 74814674G/T
3-1: 74890083A/T
3-1: 74893516T/A
3-1: 74904373T/C
3-1: 74948003C/T
3-1: 75027952G/T
3-1: 75151639A/C
3-1: 75247586T/C
3-1: 75282776G/A
3-1: 75300208T/G
3-1: 75339115A/T
3-1: 75387307A/T
3-1: 75474185T/A
3-1: 75590667T/C
3-1: 75630087T/C
3-1: 75663317T/C
3-1: 75697357C/A
3-1: 75726004T/G
3-1: 75767533A/G
3-1: 75802006A/T
3-1: 75869541T/A
3-1: 75884548G/C
3-1: 75940760C/T
3-1: 75967951T/C
3-1: 76116641C/T
3-1: 76137538C/T
3-1: 76204465A/T
3-1: 76255106T/C
3-1: 76310284C/T
3-1: 76390688T/A
3-1: 76435522G/C
3-1: 76484189G/A
3-1: 76536343C/T
3-1: 76694314A/G
3-1: 76704270T/A
3-1: 76718634A/G
3-1: 76998788C/G
3-1: 77157262A/T
3-1: 77240641A/C
3-1: 77317374T/C
3-1: 77331820C/T
3-1: 77331945A/G
3-1: 77433993A/G
3-1: 77597213T/C
3-1: 77617833A/T
3-1: 77631337G/C
3-1: 77633260T/A
3-1: 77668990T/A
3-1: 77774210T/A
3-1: 77877592A/T
3-1: 77966786C/G
3-1: 78199687A/G
3-1: 78211308T/C
3-1: 78211388A/T
3-1: 78212561G/T
3-1: 78215776T/C
3-1: 78225004A/G
3-1: 78255922A/G
3-1: 78291243G/C
3-1: 78324585C/T
3-1: 78324730C/T
3-1: 78334297C/T
3-1: 78336727A/G
3-1: 78338637G/A
3-1: 78340559G/C
3-1: 78360048G/A
3-1: 78386557A/G
3-1: 78454574C/T
3-1: 78455731G/T
3-1: 78456214C/T
3-1: 78456421G/T
3-1: 78491174C/T
3-1: 78522123G/A
3-1: 78706490G/A
3-1: 78708508T/G
3-1: 78708593C/T
3-1: 78781104T/G
3-1: 79013730G/A
3-1: 79022926T/G
3-1: 79047875T/C
3-1: 79060867A/G
3-1: 79078421A/G
3-1: 79195259C/G
3-1: 79284462C/T
3-1: 79307183A/G
3-1: 79307531C/T
3-1: 79373264A/G
3-1: 79408861C/A
3-1: 79417398G/T
3-1: 79417429G/T
3-1: 79417452G/C
3-1: 79466177T/C
3-1: 79518592T/A
3-1: 79547110G/C
3-1: 79639320G/T
3-1: 79661435T/C
3-1: 79785245C/G
3-1: 79807567T/G
3-1: 79811937C/T
3-1: 79838704T/C
3-1: 79850992T/C
3-1: 79851308T/C
3-1: 79863870T/G
3-1: 79881464G/C
3-1: 79912775T/C
3-1: 79948957C/T
3-1: 79967087C/T
3-1: 79993078A/G
3-1: 80037607A/T
3-1: 80065179T/C
3-1: 80099989A/G
3-1: 80128543C/T
3-1: 80169615A/G
3-1: 80195664T/C
3-1: 80396461G/C
3-1: 80412681A/T
3-1: 80431218T/A
3-1: 80464268T/A
3-1: 80495121A/G
3-1: 80548458G/C
3-1: 80593969A/G
3-1: 80634533G/C
3-1: 80672766T/G
3-1: 80718632A/T
3-1: 80790374C/A
3-1: 80925846G/A
3-1: 80951615C/T
3-1: 81131422C/T
3-1: 81142049C/T
3-1: 81231969C/T
3-1: 81365602A/T
3-1: 81479597C/T
3-1: 81596434T/C
3-1: 81661847C/G
3-1: 81758996T/A
3-1: 81913429A/T
3-1: 81958616T/A
3-1: 81974447G/C
3-1: 82098755G/A
3-1: 82113406A/T
3-1: 82135195C/A
3-1: 82147627T/C
3-1: 82147894G/C
3-1: 82148147T/C
3-1: 82148175A/G
3-1: 82163941G/T
3-1: 82224075C/T
3-1: 82334817G/C
3-1: 82559854A/G
3-1: 82625033C/T
3-1: 82715885A/G
3-1: 82871527T/C
3-1: 82892862G/A
3-1: 83096166C/A
3-1: 83198626T/G
3-1: 83213814C/T
3-1: 83226013G/A
3-1: 83287305T/C
3-1: 83393683A/G
3-1: 83438027G/A
3-1: 83531438A/T
3-1: 83611218G/A
3-1: 83642158A/T
3-1: 83659434C/A
3-1: 83678721A/T
3-1: 83908591T/A
3-1: 83962176G/T
3-1: 84012997T/G
3-1: 84037547T/C
3-1: 84077884C/T
3-1: 84158182T/C
3-1: 84244442C/T
3-1: 84399278G/A
3-1: 84431613C/G
3-1: 84471011G/C
3-1: 84559915T/C
3-1: 84570931T/G
3-1: 84605143G/T
3-1: 84630575C/T
3-1: 84631754C/T
3-1: 84631787T/C
3-1: 84760539G/A
3-1: 84978805C/T
3-1: 85107593A/G
3-1: 85115856C/T
3-1: 85230719T/C
3-1: 85361494T/C
3-1: 85364320G/T
3-1: 85401543C/G
3-1: 85431069C/T
3-1: 85789082T/C
3-1: 85898617C/G
3-1: 85959289G/T
3-1: 86084331G/A
3-1: 86249877C/T
3-1: 86279793A/G
3-1: 86329788G/T
3-1: 86355951G/A
3-1: 86382110T/C
3-1: 86459731G/A
3-1: 86487824A/T
3-1: 86550379A/T
3-1: 86562168G/T
3-1: 86573484A/G
3-1: 86603580T/G
3-1: 86636631G/C
3-1: 86654125A/T
3-1: 86686067T/C
3-1: 86761477A/G
3-1: 86841356T/A
3-1: 86852307T/C
3-1: 86987090T/A
3-1: 87003622T/A
3-1: 87031405G/A
3-1: 87058070C/T
3-1: 87104685A/C
3-1: 87132959C/A
3-1: 87188130T/C
3-1: 87226670A/G
3-1: 87274727C/T
3-1: 87358209G/C
3-1: 87392050C/T
3-1: 87570084A/G
3-1: 87625734T/A
3-1: 87658676T/A
3-1: 87670917G/A
3-1: 87686059G/T
3-1: 87700027A/T
3-1: 87732194G/C
3-1: 87775576T/C
3-1: 87786733C/T
3-1: 87822524A/G
3-1: 87901446G/T
3-1: 88062342A/T
3-1: 88078663A/T
3-1: 88131742C/T
3-1: 88147464T/C
3-1: 88179436G/T
3-1: 88225088A/T
3-1: 88260671G/A
3-1: 88303482C/T
3-1: 88319580A/C
3-1: 88329620C/A
3-1: 88369262G/A
3-1: 88411086A/G
3-1: 88443099T/A
3-1: 88494124A/T
3-1: 88500075A/G
3-1: 88527830T/A
3-1: 88699918G/A
3-1: 88738578T/A
3-1: 88823239G/C
3-1: 89004320A/T
3-1: 89005465G/A
3-1: 89005466A/G
3-1: 89083405G/A
3-1: 89201423T/A
3-1: 89231587A/G
3-1: 89304107C/G
3-1: 89370854G/A
3-1: 89515980C/T
3-1: 89568279C/T
3-1: 89596403A/G
3-1: 89660841C/T
3-1: 89713811T/A
3-1: 89785625T/C
3-1: 89872443A/.
3-1: 89879521A/T
3-1: 89985822A/G
3-1: 89990939C/T
3-1: 90095937A/G
3-1: 90130874A/G
3-1: 90199045A/G
3-1: 90232384C/T
3-1: 90240931G/T
3-1: 90240942G/A
3-1: 90325977T/C
3-1: 90346211A/G
3-1: 90385202G/A
3-1: 90431941C/T
3-1: 90514040A/G
3-1: 90524472A/G
3-1: 90524528G/A
3-1: 90585866A/G
3-1: 90616247T/C
3-1: 90652888C/A
3-1: 90679889A/T
3-1: 90731598G/A
3-1: 90785749T/A
3-1: 90836784A/T
3-1: 90892910A/T
3-1: 90900327C/G
3-1: 90903187G/A
3-1: 90997531T/C
3-1: 91089940G/A
3-1: 91269305G/T
3-1: 91406318A/T
3-1: 91628793C/T
3-1: 91829201T/A
3-1: 91879595A/G
3-1: 91968078C/T
3-1: 91995874G/C
3-1: 92107362A/G
3-1: 92169056G/A
3-1: 92508002T/G
3-1: 92615705T/C
3-1: 92623801A/G
3-1: 92632464A/T
3-1: 92736895T/C
3-1: 92851496C/A
3-1: 92922749T/A
3-1: 93005686T/A
3-1: 93159377T/C
3-1: 93271623A/C
3-1: 93317206A/C
3-1: 93320456G/T
3-1: 93321520A/T
3-1: 93337748A/G
3-1: 93353311A/G
3-1: 93353470G/A
3-1: 93355141T/C
3-1: 93355456C/T
3-1: 93359038C/T
3-1: 93360253T/C
3-1: 93360412A/G
3-1: 93364699T/A
3-1: 93537177C/T
3-1: 93701511T/G
3-1: 93845585T/C
3-1: 93845662C/T
3-1: 93856077A/T
3-1: 93931201C/T
3-1: 94028407C/T
3-1: 94028524A/T
3-1: 94028789A/G
3-1: 94051972G/T
3-1: 94052033A/G
3-1: 94052067A/G
3-1: 94052365T/A
3-1: 94108079G/A
3-1: 94118433A/G
3-1: 94528589T/C
3-1: 94620325G/A
3-1: 94672294A/T
3-2: 7421A/G
3-2: 182499T/C
3-2: 201640T/C
3-2: 424572A/T
3-2: 449578G/T
3-2: 497529A/T
3-2: 554247C/A
3-2: 725365T/C
3-2: 811283T/C
3-2: 880483A/T
3-2: 976099C/A
3-2: 1220367G/T
3-2: 1240239C/T
3-2: 1267427C/T
3-2: 1313329G/A
3-2: 1375022C/T
3-2: 1376681T/G
3-2: 1401874G/T
3-2: 1440189G/A
3-2: 1684283T/A
3-2: 1858465T/C
3-2: 1992228A/G
3-2: 2027272C/T
3-2: 2042292A/G
3-2: 2167720C/T
3-2: 2170648A/T
3-2: 2207860A/C
3-2: 2240635T/C
3-2: 2454879A/T
3-2: 2456710A/T
3-2: 2467203G/A
3-2: 2470973G/A
3-2: 2471161G/A
3-2: 2490675A/T
3-2: 2609947T/G
3-2: 2680283G/C
3-2: 2729850G/A
3-2: 2730064T/A
3-2: 2740149T/A
3-2: 2755909G/C
3-2: 2992577T/C
3-2: 3129337G/A
3-2: 3139860C/T
3-2: 3152640C/T
3-2: 3270973C/G
3-2: 3893983C/A
3-2: 4017120A/G
3-2: 4033476C/A
3-2: 4125469C/T
3-2: 4193290A/T
3-2: 4458982C/T
3-2: 4966671T/A
3-2: 5107746G/C
3-2: 5128882C/A
3-2: 5158125C/T
3-2: 5231184C/A
3-2: 5266922G/A
3-2: 5380810A/G
3-2: 5407468A/G
3-2: 5453224C/T
3-2: 5468568T/A
3-2: 5576982A/T
3-2: 5589480C/T
3-2: 5603713G/A
3-2: 5696919T/C
3-2: 5702341C/T
3-2: 5747297G/A
3-2: 5773496C/T
3-2: 5831274C/G
3-2: 5909325G/A
3-2: 5958387C/T
3-2: 6143263A/T
3-2: 6145186A/C
3-2: 6245510T/A
3-2: 6269677A/T
3-2: 6270836C/G
3-2: 6270846G/A
3-2: 6302766C/T
3-2: 6449272A/G
3-2: 6451083T/A
3-2: 6460035G/A
3-2: 6503565C/T
3-2: 6566023T/C
3-2: 6627956T/C
3-2: 6684226A/C
3-2: 6738308A/T
3-2: 6753515C/A
3-2: 6765701C/A
3-2: 6868430A/G
3-2: 6939852C/T
3-2: 7008701A/T
3-2: 7089833A/T
3-2: 7180187G/A
3-2: 7180610G/T
3-2: 7181204T/A
3-2: 7201044C/T
3-2: 7217286A/G
3-2: 7241644G/C
3-2: 7259803A/T
3-2: 7266356A/C
3-2: 7266472A/T
3-2: 7271391G/T
3-2: 7271569A/G
3-2: 7286375T/C
3-2: 7317286C/T
3-2: 7333776C/T
3-2: 7339829T/C
3-2: 7424617G/C
3-2: 7501918A/G
3-2: 7506316G/T
3-2: 7520994T/A
3-2: 7521260T/C
3-2: 7530986C/T
3-2: 7531466T/A
3-2: 7558504T/C
3-2: 7561624A/T
3-2: 7583090A/T
3-2: 7589934T/A
3-2: 7600154G/C
3-2: 7689898A/G
3-2: 7694901A/C
3-2: 7710462G/T
3-2: 7711813G/C
3-2: 7712019G/A
3-2: 7712335C/T
3-2: 7759289T/C
3-2: 7815429A/G
3-2: 7882851C/T
3-2: 7897996T/C
3-2: 7953396T/C
3-2: 8073433G/A
3-2: 8134578T/C
3-2: 8170643G/A
3-2: 8256605A/G
3-2: 8281383T/G
3-2: 8294746G/A
3-2: 8435035C/T
3-2: 8439634A/T
3-2: 8461274C/T
3-2: 8471300G/A
3-2: 8514511C/A
3-2: 8580239C/T
3-2: 8581818G/A
3-2: 8628819G/A
3-2: 8631323T/A
3-2: 8663305A/C
3-2: 8815817C/A
3-2: 8887311A/G
3-2: 8985554G/A
3-2: 9044421T/A
3-2: 9092094A/G
3-2: 9097536G/T
3-2: 9097987G/C
3-2: 9142977C/T
3-2: 9171716T/A
3-2: 9230031C/T
3-2: 9260891C/A
3-2: 9260924G/A
3-2: 9260932A/G
3-2: 9260942A/G
3-2: 9367494C/G
3-2: 9571508G/C
3-2: 9692883A/G
3-2: 9720553C/T
3-2: 9745722A/C
3-2: 9757810C/T
3-2: 9791481A/G
3-2: 9806178T/A
3-2: 9820703G/A
3-2: 9849440T/A
3-2: 9909208G/A
3-2: 9935258C/T
3-2: 9972634C/G
3-2: 10116056A/G
3-2: 10331039C/T
3-2: 10339781T/G
3-2: 10341667C/G
3-2: 10391713T/C
3-2: 10503706C/T
3-2: 10558006A/G
3-2: 10686867C/T
3-2: 10918211A/T
3-2: 10935896A/G
3-2: 10998217C/A
3-2: 11001491A/T
3-2: 11018632A/G
3-2: 11204982T/C
3-2: 11435388T/C
3-2: 11589564C/A
3-2: 11670602C/A
3-2: 11728185C/T
3-2: 11779517T/C
3-2: 11821519A/G
3-2: 11858700G/A
3-2: 11894031T/C
3-2: 11905413C/T
3-2: 11917503A/T
3-2: 11996862G/A
3-2: 12016415G/A
3-2: 12115889G/A
3-2: 12148852A/T
3-2: 12185172A/T
3-2: 12190428T/C
3-2: 12190447A/G
3-2: 12190479G/A
3-2: 12191984G/C
3-2: 12192006T/C
3-2: 12192093T/A
3-2: 12203441C/A
3-2: 12203545A/G
3-2: 12273137T/G
3-2: 12340770A/T
3-2: 12386556A/T
3-2: 12387755A/C
3-2: 12399274G/A
3-2: 12431865G/A
3-2: 12445062T/G
3-2: 12447576A/T
3-2: 12456963A/G
3-2: 12504351A/G
3-2: 12538705A/G
3-2: 12578507A/C
3-2: 12627028G/A
3-2: 12687519A/G
3-2: 12781400G/T
3-2: 12805875A/C
3-2: 12835909C/T
3-2: 12846029C/A
3-2: 12863366T/C
3-2: 12912475G/A
3-2: 12976710A/G
3-2: 13182882G/A
3-2: 13190176C/T
3-2: 13196095G/A
3-2: 13282041C/T
3-2: 13313393C/G
3-2: 13390743A/G
3-2: 13436659G/A
3-2: 13454040A/T
3-2: 13502376A/C
3-2: 13517637A/G
3-2: 13530699G/A
3-2: 13551389T/C
3-2: 13557049T/C
3-2: 13557561G/A
3-2: 13557916A/T
3-2: 13602121T/C
3-2: 13743992G/T
3-2: 13892190C/T
3-2: 13918107C/T
3-2: 13943302G/A
3-2: 13994842C/T
3-2: 14042241A/T
3-2: 14043392C/T
3-2: 14045615A/G
3-2: 14065413A/G
3-2: 14086799A/G
3-2: 14139424A/G
3-2: 14201416A/T
3-2: 14261052A/G
3-2: 14290431C/T
3-2: 14433481G/A
3-2: 14606364T/G
3-2: 14708770G/A
3-2: 14784330G/C
3-2: 14834164T/A
3-2: 14899963C/T
3-2: 14901001C/T
3-2: 14901169G/A
3-2: 14901462C/T
3-2: 14916947A/C
3-2: 14933967A/G
3-2: 15047838C/T
3-2: 15062762G/A
3-2: 15102908T/G
3-2: 15148581G/A
3-2: 15279107T/C
3-2: 15279761T/A
3-2: 15282053C/T
3-2: 15282060A/G
3-2: 15282231C/T
3-2: 15289204G/A
3-2: 15323645A/C
3-2: 15349574G/A
3-2: 15386909T/C
3-2: 15404675T/C
3-2: 15450120C/T
3-2: 15500460C/G
3-2: 15546323A/G
3-2: 15660543A/T
3-2: 15705393G/T
3-2: 15745571C/A
3-2: 15745720C/A
3-2: 15798913A/G
3-2: 15834449G/A
3-2: 15849270C/A
3-2: 15873203A/G
3-2: 15977449G/A
3-2: 16099760G/A
3-2: 16100036G/T
3-2: 16125389C/A
3-2: 16133602A/T
3-2: 16159581G/T
3-2: 16207046A/C
3-2: 16214415C/T
3-2: 16268491T/A
3-2: 16342242T/G
3-2: 16400204G/A
3-2: 16437193T/G
3-2: 16458088G/C
3-2: 16472844A/G
3-2: 16484921G/A
3-2: 16486219T/A
3-2: 16539258G/C
3-2: 16565225C/T
3-2: 16625510A/G
3-2: 16635683T/C
3-2: 16654147T/C
3-2: 16665482A/C
3-2: 16746490G/T
3-2: 16748347A/G
3-2: 16764226C/T
3-2: 16772753G/A
3-2: 16773970C/T
3-2: 16774136A/T
3-2: 16774427A/C
3-2: 16774430G/T
3-2: 16779794G/T
3-2: 16788467T/C
3-2: 16807967C/A
3-2: 16833329A/G
3-2: 16847945A/T
3-2: 16866221T/A
3-2: 16872542G/A
3-2: 16889553C/T
3-2: 17038421G/C
3-2: 17048561C/T
3-2: 17051019C/A
3-2: 17175018C/T
3-2: 17182789T/C
3-2: 17214461C/T
3-2: 17251104G/A
3-2: 17351671A/G
3-2: 17378525C/A
3-2: 17481488T/C
3-2: 17533067G/A
3-2: 17690312G/A
3-2: 17733836A/G
3-2: 17769558T/A
3-2: 17857854G/A
3-2: 17893549C/T
3-2: 17902491A/C
3-2: 17903761G/A
3-2: 17925597A/G
3-2: 17937101A/T
3-2: 17951165G/A
3-2: 18019798C/T
3-2: 18073990C/A
3-2: 18094832T/C
3-2: 18116346T/A
3-2: 18137892G/A
3-2: 18140045C/T
3-2: 18148712T/C
3-2: 18150597A/T
3-2: 18151534A/C
3-2: 18152363T/C
3-2: 18152587G/A
3-2: 18153541T/C
3-2: 18154107A/G
3-2: 18169968C/T
3-2: 18185658C/T
3-2: 18205258G/T
3-2: 18215608T/C
3-2: 18255359C/T
3-2: 18273468T/G
3-2: 18281972T/C
3-2: 18364741A/G
3-2: 18364762T/A
3-2: 18365996C/G
3-2: 18372813T/C
3-2: 18564172C/T
3-2: 18566913C/G
3-2: 18588362T/C
3-2: 18590271G/A
3-2: 18604501A/G
3-2: 18830846G/T
3-2: 18832049G/A
3-2: 18881877A/G
3-2: 18895601C/T
3-2: 18925434C/T
3-2: 18958926G/A
3-2: 19125948G/C
3-2: 19147350G/A
3-2: 19162919A/T
3-2: 19258803G/A
3-2: 19286074T/A
3-2: 19303923C/T
3-2: 19379892A/C
3-2: 19398767T/G
3-2: 19409214C/T
3-2: 19420025G/A
3-2: 19515136G/A
3-2: 19525217C/A
3-2: 19536645G/A
3-2: 19601723C/T
3-2: 19743341A/T
3-2: 19786904A/T
3-2: 19806968G/A
3-2: 19836398A/G
3-2: 19878230T/C
3-2: 19879265T/G
3-2: 19881643C/A
3-2: 19881748C/T
3-2: 19881906T/C
3-2: 19882398C/T
3-2: 19886021C/T
3-2: 19892811A/G
3-2: 20069373G/A
3-2: 20077072A/G
3-2: 20101302C/T
3-2: 20129510C/T
3-2: 20246904T/C
3-2: 20283218A/T
3-2: 20297645C/T
3-2: 20393127T/A
3-2: 20445172T/C
3-2: 20458616G/T
3-2: 20483013A/C
3-2: 20500248T/G
3-2: 20524095A/C
3-2: 20559210G/A
3-2: 20573883T/C
3-2: 20582032G/A
3-2: 20603726C/T
3-2: 20621080T/C
3-2: 20627175T/C
3-2: 20644347A/T
3-2: 20655999A/C
3-2: 20657360G/A
3-2: 20676394C/A
3-2: 20687010T/C
3-2: 20785672G/A
3-2: 20824746G/C
3-2: 20901449A/G
3-2: 20909674A/G
3-2: 20933132A/G
3-2: 20961050G/A
3-2: 21004065A/C
3-2: 21030711G/T
3-2: 21032043C/T
3-2: 21033179T/G
3-2: 21069060A/G
3-2: 21071661T/C
3-2: 21074443C/T
3-2: 21123727C/A
3-2: 21186373C/T
3-2: 21215711T/C
3-2: 21217965G/C
3-2: 21217989A/G
3-2: 21218093C/A
3-2: 21220380T/A
3-2: 21225761C/A
3-2: 21227687C/T
3-2: 21228610G/A
3-2: 21228751C/T
3-2: 21229122A/T
3-2: 21374363T/C
3-2: 21381253G/A
3-2: 21472842T/C
3-2: 21548449C/A
3-2: 21650996T/C
3-2: 21655161T/C
3-2: 21690420A/G
3-2: 21739828T/A
3-2: 21752868T/A
3-2: 21800684G/A
3-2: 21888251C/T
3-2: 21909970T/C
3-2: 22071365A/T
3-2: 22074238G/A
3-2: 22177445A/G
3-2: 22268746T/A
3-2: 22359785G/A
3-2: 22423852C/A
3-2: 22455156A/C
3-2: 22535729T/A
3-2: 22562730T/C
3-2: 22581247A/C
3-2: 22595277T/C
3-2: 22608044A/T
3-2: 22635321G/T
3-2: 22651418A/G
3-2: 22665985C/T
3-2: 22695634C/G
3-2: 22708169G/A
3-2: 22720767A/T
3-2: 22732002T/G
3-2: 22745214A/T
3-2: 22756221A/G
3-2: 22822486A/C
3-2: 22968927A/G
3-2: 23151497C/T
3-2: 23249967A/G
3-2: 23255194C/T
3-2: 23255309C/A
3-2: 23255413T/C
3-2: 23255417T/G
3-2: 23268081G/C
3-2: 23278158A/G
3-2: 23280910G/A
3-2: 23294220T/A
3-2: 23328893G/T
3-2: 23345118C/T
3-2: 23409716T/C
3-2: 23437616A/G
3-2: 23647932T/C
3-2: 23655442G/A
3-2: 23724478A/T
3-2: 23734886G/A
3-2: 23774592G/A
3-2: 23819643G/A
3-2: 23883218G/C
3-2: 23940768A/G
3-2: 23978255G/A
3-2: 23989668A/G
3-2: 24001406C/T
3-2: 24038097C/A
3-2: 24144322A/T
3-2: 24192511A/G
3-2: 24302205A/G
3-2: 24389174C/T
3-2: 24590347C/T
3-2: 24601830C/A
3-2: 24619587T/C
3-2: 24621297A/G
3-2: 24633290T/A
3-2: 24638628C/G
3-2: 24639177C/T
3-2: 24639297T/G
3-2: 24639319G/A
3-2: 24650417T/C
3-2: 24650710C/T
3-2: 24688461T/C
3-2: 24737605C/T
3-2: 24768839A/G
3-2: 24828048T/C
3-2: 24829723T/A
3-2: 24830382G/T
3-2: 24841881A/G
3-2: 24845739C/A
3-2: 24966470T/C
3-2: 24980718C/A
3-2: 25058193C/T
3-2: 25137829G/A
3-2: 25164628C/A
3-2: 25208649T/A
3-2: 25270202A/T
3-2: 25279551G/A
3-2: 25315777T/C
3-2: 25332481T/G
3-2: 25343962A/G
3-2: 25408513A/C
3-2: 25410467T/C
3-2: 25445483T/C
3-2: 25467930A/C
3-2: 25499538G/T
3-2: 25535156C/T
3-2: 25545318C/T
3-2: 25571305T/C
3-2: 25650029G/A
3-2: 25681134A/G
3-2: 25723061A/T
3-2: 25752583A/G
3-2: 25796990T/C
3-2: 25883685A/G
3-2: 25934892A/G
3-2: 25937163T/A
3-2: 25944160T/C
3-2: 25945841T/C
3-2: 25945992T/C
3-2: 25946050G/A
3-2: 25949175C/T
3-2: 25963400T/C
3-2: 26045835G/A
3-2: 26066411T/C
3-2: 26180154A/G
3-2: 26232167G/A
3-2: 26251352A/G
3-2: 26293108A/G
3-2: 26294110C/T
3-2: 26355743C/T
3-2: 26436653G/C
3-2: 26453868A/C
3-2: 26521887T/A
3-2: 26651652A/G
3-2: 26814586C/A
3-2: 26870109T/A
3-2: 26906860C/A
3-2: 26919771A/G
3-2: 26925082C/T
3-2: 26943787T/A
3-2: 27051144C/T
3-2: 27079522C/T
3-2: 27126456A/G
3-2: 27192241T/A
3-2: 27223525T/C
3-2: 27272340A/T
3-2: 27281063T/C
3-2: 27290528G/T
3-2: 27338797T/C
3-2: 27383061A/T
3-2: 27396487A/G
3-2: 27397458C/T
3-2: 27397572T/G
3-2: 27397673A/G
3-2: 27413235T/C
3-2: 27464800C/T
3-2: 27502015A/T
3-2: 27520003T/C
3-2: 27625751T/C
3-2: 27627574T/C
3-2: 27682793T/A
3-2: 27698765G/A
3-2: 27709243G/A
3-2: 27720827T/G
3-2: 27776852T/C
3-2: 27780741G/T
3-2: 27788793T/C
3-2: 27945109A/G
3-2: 27957189T/C
3-2: 27977300C/A
3-2: 27988078A/C
3-2: 28008592A/G
3-2: 28301341G/A
3-2: 28444638A/G
3-2: 28512325C/T
3-2: 28665082T/A
3-2: 28725829A/T
3-2: 28738572C/T
3-2: 28775918G/A
3-2: 28924330C/T
3-2: 29023653G/A
3-2: 29036611T/A
3-2: 29056791T/A
3-2: 29095040T/G
3-2: 29104435C/T
3-2: 29127033G/T
3-2: 29161872T/C
3-2: 29219918A/G
3-2: 29251061T/C
3-2: 29306920G/A
3-2: 29459862A/G
3-2: 29481546A/G
3-2: 29556352A/G
3-2: 29647353A/G
3-2: 29699616T/A
3-2: 29781901A/G
3-2: 29842511T/C
3-2: 29873078C/T
3-2: 29888044G/C
3-2: 29981071C/T
3-2: 30048160A/C
3-2: 30363454G/T
3-2: 30604973A/C
3-2: 30665521C/T
3-2: 30686552G/A
3-2: 30741115A/G
3-2: 30784678A/C
3-2: 30813882G/T
3-2: 30852787A/G
3-2: 30879492A/T
3-2: 30910594C/A
3-2: 30941618G/A
3-2: 30944755T/C
3-2: 31028500A/T
3-2: 31037134G/T
3-2: 31053993C/A
3-2: 31064117T/G
3-2: 31102534G/A
3-2: 31157535C/T
3-2: 31217859A/C
3-2: 31276714G/A
3-2: 31312986A/G
3-2: 31491930G/A
3-2: 31567094G/A
3-2: 31609510T/C
3-2: 31621520A/C
3-2: 31686791T/G
3-2: 31719282G/A
3-2: 31771911T/G
3-2: 31799403T/A
3-2: 31807418C/T
3-2: 31844598T/C
3-2: 31892331C/T
3-2: 31985148A/T
3-2: 32135091G/A
3-2: 32148599G/A
3-2: 32172536G/C
3-2: 32207430C/T
3-2: 32219215A/G
3-2: 32268953G/A
3-2: 32318006T/C
3-2: 32404651A/C
3-2: 32438924C/T
3-2: 32457388A/T
3-2: 32671233T/C
3-2: 32741879T/C
3-2: 32765906C/T
3-2: 32797596T/G
3-2: 32831950G/A
3-2: 32887821C/T
3-2: 32936229C/T
3-2: 32946406G/T
3-2: 33065429G/A
3-2: 33163605T/A
3-2: 33270032G/T
3-2: 33586723G/A
3-2: 33775539T/C
3-2: 33792409C/G
3-2: 33899895T/A
3-2: 33900992A/T
3-2: 33904939T/A
3-2: 33907649G/A
3-2: 33922133G/A
3-2: 33922471A/G
3-2: 33948519T/A
3-2: 33970224A/G
3-2: 33991394G/A
3-2: 34040499C/T
3-2: 34050954G/A
3-2: 34263773T/C
3-2: 34292095A/C
3-2: 34377088C/T
3-2: 34412447A/G
3-2: 34448960T/G
3-2: 34547236T/A
3-2: 34595175A/G
3-2: 34765820G/A
3-2: 34793563T/A
3-2: 34843814A/T
3-2: 34857429C/G
3-2: 34868529T/C
3-2: 34879870T/C
3-2: 34897052A/G
3-2: 34928873C/T
3-2: 34985150G/A
3-2: 34992150C/T
3-2: 35000059G/A
3-2: 35402673C/T
3-2: 35475222T/C
3-2: 35541341C/T
3-2: 35554508T/C
3-2: 35677703C/A
3-2: 35681963A/G
3-2: 35699213A/G
3-2: 35712629A/G
3-2: 35715092T/C
3-2: 35715786G/C
3-2: 35722073T/A
3-2: 35722183G/A
3-2: 35723736A/G
3-2: 35724114T/G
3-2: 35724690A/C
3-2: 35724826T/C
3-2: 35735685G/A
3-2: 35746269A/T
3-2: 35839456T/A
3-2: 35986479T/C
3-2: 36273225C/T
3-2: 36273893G/T
3-2: 36302293A/T
3-2: 36334095G/T
3-2: 36369796G/T
3-2: 36401037A/G
3-2: 36411153G/T
3-2: 36424594G/A
3-2: 36520732C/T
3-2: 36550652T/C
3-2: 36719111A/C
3-2: 36721256C/T
3-2: 36844249C/T
3-2: 36857164G/C
3-2: 37031938A/C
3-2: 37126959A/T
3-2: 37210822G/A
3-2: 37245906T/A
3-2: 37309174G/A
3-2: 37351397G/A
3-2: 37393980A/G
3-2: 37495711G/A
3-2: 37516613C/T
3-2: 37542226C/T
3-2: 37723568C/T
3-2: 37737415T/C
3-2: 37998953G/A
3-2: 38002410C/G
3-2: 38023038C/T
3-2: 38121882T/A
3-2: 38150531G/A
3-2: 38171098A/T
3-2: 38252123A/C
3-2: 38262442G/T
3-2: 38391743C/T
3-2: 38434234T/C
3-2: 38448183T/G
3-2: 38504889G/A
3-2: 38673909G/A
3-2: 38792966T/C
3-2: 38802007G/A
3-2: 38929852T/A
3-2: 38978951A/G
3-2: 39054686T/C
3-2: 39099909A/T
3-2: 39162131A/C
3-2: 39192667G/A
3-2: 39318236G/A
3-2: 39327232A/G
3-2: 39333589G/C
3-2: 39412126G/C
3-2: 39525308T/C
3-2: 39597185A/G
3-2: 39654775C/T
3-2: 39703181T/A
3-2: 39762108C/T
3-2: 39783946C/A
3-2: 40040861T/G
3-2: 40055982C/T
3-2: 40056128C/A
3-2: 40364476T/C
3-2: 40456200A/G
3-2: 40470065T/G
3-2: 40609498C/A
3-2: 40697017G/T
3-2: 40706011T/C
3-2: 40723448G/T
3-2: 40803072G/T
3-2: 40838199T/C
3-2: 40934236C/T
3-2: 40949582T/C
3-2: 40950371T/C
3-2: 40951613A/G
3-2: 40952387T/C
3-2: 40953220G/C
3-2: 40953412T/C
3-2: 40953675T/G
3-2: 40953735G/T
3-2: 40955308T/C
3-2: 40956060T/C
3-2: 40958287G/A
3-2: 40960416G/C
3-2: 41019066A/T
3-2: 41085650A/T
3-2: 41115759A/G
3-2: 41146424C/A
3-2: 41257283C/A
3-2: 41287879G/A
3-2: 41300734A/T
3-2: 41337123C/A
3-2: 41373436A/T
3-2: 41413102A/G
3-2: 41449770G/T
3-2: 41468779C/A
3-2: 41640928C/G
3-2: 41707889A/G
3-2: 41901338G/T
3-2: 41919168C/T
3-2: 41984331T/C
3-2: 42054839G/A
3-2: 42082271T/C
3-2: 42093300T/C
3-2: 42126693T/C
3-2: 42196840T/C
3-2: 42215355A/G
3-2: 42302059G/A
3-2: 42306302C/T
3-2: 42391056A/G
3-2: 42409760G/A
3-2: 42411600A/T
3-2: 42414794A/G
3-2: 42428578G/A
3-2: 42450110G/C
3-2: 42473407G/A
3-2: 42512547T/C
3-2: 42613835C/T
3-2: 42638702T/C
3-2: 42665879G/C
3-2: 42668221C/G
3-2: 42744444A/C
3-2: 42851864T/G
3-2: 42928869G/A
3-2: 43100860C/A
3-2: 43106199G/A
3-2: 43106589T/C
3-2: 43106932T/A
3-2: 43114861T/C
3-2: 43172164A/C
3-2: 43291407G/T
3-2: 43331239G/A
3-2: 43508980G/A
3-2: 43577702T/G
3-2: 43715077G/A
3-2: 43794852C/T
3-2: 43920652A/C
3-2: 43962419G/C
3-2: 44056167T/A
3-2: 44071878A/G
3-2: 44136363A/G
3-2: 44360829G/A
3-2: 44462961T/A
3-2: 44568360T/A
3-2: 44589353T/C
3-2: 44686034C/T
3-2: 44711545T/C
3-2: 44820097G/C
3-2: 44917165C/A
3-2: 44930070T/C
3-2: 44959583G/A
3-2: 45031914C/T
3-2: 45091861T/A
3-2: 45127337T/G
3-2: 45145522A/T
3-2: 45263108A/T
3-2: 45338919C/T
3-2: 45487836C/T
3-2: 45508195T/A
3-2: 45537720C/T
3-2: 45572889A/C
3-2: 45600259T/C
3-2: 45744821C/A
3-2: 45776941C/T
3-2: 45851887T/A
3-2: 45883998A/G
3-2: 45884597T/C
3-2: 45884631A/G
3-2: 45884709G/A
3-2: 45885375C/A
3-2: 45885406G/C
3-2: 45885522T/A
3-2: 45889760T/C
3-2: 45889895G/T
3-2: 45903932A/G
3-2: 45989905T/A
3-2: 46011289C/T
3-2: 46044661C/T
3-2: 46087331A/G
3-2: 46118736A/G
3-2: 46201085T/G
3-2: 46355433A/T
3-2: 46360412G/A
3-2: 46379376G/A
3-2: 46465200G/A
3-2: 46535697G/A
3-2: 46570952T/C
3-2: 46656921G/A
3-2: 46749718A/G
3-2: 46902574C/T
3-2: 46954392A/T
3-2: 47054334C/A
3-2: 47068003T/C
3-2: 47141422C/T
3-2: 47275160G/A
3-2: 47279294C/G
3-2: 47331238C/T
3-2: 47368069G/A
3-2: 47393248A/C
3-2: 47453550G/T
3-2: 47530069C/T
3-2: 47601996A/G
3-2: 47616142A/C
3-2: 47617426A/T
3-2: 47649906G/T
3-2: 47658027G/A
3-2: 47682087C/T
3-2: 47704314T/C
3-2: 47714802A/G
3-2: 47723767T/C
3-2: 47732219G/A
3-2: 47768849C/A
3-2: 47775296C/T
3-2: 47783833T/C
3-2: 47783935A/G
3-2: 47784218G/T
3-2: 47784889T/C
3-2: 47785328A/G
3-2: 47786224A/G
3-2: 47786790T/C
3-2: 47787476G/C
3-2: 47787667G/C
3-2: 47794337C/T
3-2: 47794850G/A
3-2: 47803665G/A
3-2: 47807113G/A
3-2: 47817984T/C
3-2: 47828489T/C
3-2: 47829142T/A
3-2: 47829288A/G
3-2: 47837254T/C
3-2: 47838696T/A
3-2: 47856331T/C
3-2: 47860489T/C
3-2: 47867310T/A
3-2: 47867470G/A
3-2: 47871003C/T
3-2: 47871027T/C
3-2: 47958502C/T
3-2: 47977404C/T
3-2: 47987557G/T
3-2: 48001054C/T
3-2: 48067399T/G
3-2: 48093615T/A
3-2: 48137791T/A
3-2: 48192335T/C
3-2: 48203913C/T
3-2: 48227153T/A
3-2: 48239370C/T
3-2: 48260309G/A
3-2: 48271708C/T
3-2: 48320856A/T
3-2: 48321235A/T
3-2: 48322251C/A
3-2: 48337284G/A
3-2: 48399968C/A
3-2: 48430289T/G
3-2: 48452651C/T
3-2: 48583601A/T
3-2: 48660272G/A
3-2: 48683978G/A
3-2: 48742716G/A
3-2: 48756248T/C
3-2: 48848983G/T
3-2: 48861704A/T
3-2: 48942156T/A
3-2: 49005709G/C
3-2: 49006777G/A
3-2: 49006888A/G
3-2: 49025979G/A
3-2: 49067864C/T
3-2: 49078244C/G
3-2: 49129816T/G
3-2: 49202156T/A
3-2: 49204670C/T
3-2: 49276520A/T
3-2: 49327426G/A
3-2: 49355694A/G
3-2: 49390856T/A
3-2: 49416664C/T
3-2: 49432066T/C
3-2: 49569869G/A
3-2: 49572146G/A
3-2: 49578780G/A
3-2: 49578873A/G
3-2: 49732875T/G
3-2: 49760791T/A
3-2: 49826718G/A
3-2: 49839975G/T
3-2: 49845120A/C
3-2: 49881772G/A
3-2: 49970210C/A
3-2: 49970218C/T
3-2: 49997665C/T
3-2: 50025772T/C
3-2: 50026198A/T
3-2: 50054287A/G
3-2: 50054588A/T
3-2: 50077819C/T
3-2: 50122837C/T
3-2: 50220693A/G
3-2: 50396953C/A
3-2: 50443772T/A
3-2: 50454055G/A
3-2: 50465952C/T
3-2: 50476938T/C
3-2: 50499259T/C
3-2: 50624717T/C
3-2: 50737113G/A
3-2: 50754932C/T
3-2: 50794432G/T
3-2: 50815910C/T
3-2: 50987697T/C
3-2: 51039596A/T
3-2: 51062158G/T
3-2: 51166917T/C
3-2: 51191590A/G
3-2: 51208539G/T
3-2: 51224066T/C
3-2: 51234310A/G
3-2: 51235172T/C
3-2: 51244517C/T
3-2: 51288553C/A
3-2: 51292524G/T
3-2: 51292625T/G
3-2: 51293047G/A
3-2: 51293067C/A
3-2: 51293167T/C
3-2: 51293169T/C
3-2: 51293796T/G
3-2: 51293816C/T
3-2: 51294767A/T
3-2: 51295276G/C
3-2: 51304918T/A
3-2: 51305230C/T
3-2: 51330196C/A
3-2: 51348434A/G
3-2: 51360536A/G
3-2: 51453658C/T
3-2: 51550354T/A
3-2: 51551483C/T
3-2: 51565129A/G
3-2: 51576686A/G
3-2: 51619970A/T
3-2: 51637194A/G
3-2: 51727528T/A
3-2: 51730398A/G
3-2: 51747984C/T
3-2: 51749086G/A
3-2: 51759246C/T
3-2: 52008037T/C
3-2: 52072506C/T
3-2: 52073728T/C
3-2: 52116442A/T
3-2: 52126523G/A
3-2: 52129824G/A
3-2: 52165929G/C
3-2: 52200554C/G
3-2: 52295322A/T
3-2: 52297348A/T
3-2: 52297369T/C
3-2: 52372289C/T
3-2: 52411741G/T
3-2: 52415251T/C
3-2: 52415333A/T
3-2: 52415424G/C
3-2: 52415571T/A
3-2: 52416365A/C
3-2: 52435816G/C
3-2: 52472401C/A
3-2: 52486119T/A
3-2: 52562234G/A
3-2: 52606633C/T
3-2: 52656333T/C
3-2: 52683201T/C
3-2: 52721806T/G
3-2: 52752414C/G
3-2: 52771202T/C
3-2: 52781216G/A
3-2: 52810371G/T
3-2: 52811683C/T
3-2: 52859230A/G
3-2: 52861593A/T
3-2: 52865189A/G
3-2: 52865615G/T
3-2: 52917289A/G
3-2: 52942606C/A
3-2: 53017426C/A
3-2: 53037352C/T
3-2: 53084048T/C
3-2: 53096706C/T
3-2: 53097921C/T
3-2: 53106555G/A
3-2: 53108646C/T
3-2: 53109058C/A
3-2: 53119735C/T
3-2: 53130791T/C
3-2: 53136390T/A
3-2: 53141644G/A
3-2: 53141899A/G
3-2: 53141906C/A
3-2: 53141914G/A
3-2: 53142023A/G
3-2: 53148664T/C
3-2: 53148884T/A
3-2: 53148897A/G
3-2: 53156745A/G
3-2: 53165151T/C
3-2: 53165331A/.
3-2: 53171472T/C
3-2: 53181747G/A
3-2: 53182547T/C
3-2: 53182859A/G
3-2: 53182918G/A
3-2: 53183635T/C
3-2: 53183963G/A
3-2: 53184077G/A
3-2: 53184531A/G
3-2: 53184644G/T
3-2: 53185842G/A
3-2: 53185858T/C
3-2: 53185869A/G
3-2: 53185882T/G
3-2: 53185883T/C
3-2: 53185905A/G
3-2: 53185909T/.
3-2: 53185934C/T
3-2: 53185950A/.
3-2: 53185951C/.
3-2: 53185954C/T
3-2: 53185959C/T
3-2: 53185965C/T
3-2: 53185986G/A
3-2: 53186017A/T
3-2: 53186025C/A
3-2: 53186033C/G
3-2: 53186036G/T
3-2: 53186037A/T
3-2: 53186049C/T
3-2: 53186058G/.
3-2: 53186070G/C
3-2: 53186080T/C
3-2: 53186091A/G
3-2: 53186096C/T
3-2: 53186204G/A
3-2: 53186213A/.
3-2: 53186220G/A
3-2: 53186227A/T
3-2: 53186239G/A
3-2: 53186248C/T
3-2: 53186261G/A
3-2: 53186265A/C
3-2: 53186289C/T
3-2: 53186291C/.
3-2: 53186300A/T
3-2: 53186305A/.
3-2: 53186306T/C
3-2: 53192178T/A
3-2: 53198101G/A
3-2: 53205427G/A
3-2: 53208248T/A
3-2: 53208335G/A
3-2: 53228066T/G
3-2: 53239783C/T
3-2: 53254411G/A
3-2: 53264941A/C
3-2: 53275284C/G
3-2: 53312909C/T
3-2: 53354493T/C
3-2: 53392272C/T
3-2: 53474553T/C
3-2: 53501418T/C
3-2: 53511659T/A
3-2: 53523961C/T
3-2: 53614390G/A
3-2: 53639299G/C
3-2: 53650190A/G
3-2: 53670648A/T
3-2: 53673618G/A
3-2: 53691453A/T
3-2: 53691605T/C
3-2: 53691634G/A
3-2: 53691822T/C
3-2: 53693046G/C
3-2: 53721574C/A
3-2: 53758035A/G
3-2: 53795532G/A
3-2: 53892336A/G
3-2: 53895870G/A
3-2: 53924664G/A
3-2: 53971400G/A
3-2: 54056590T/C
3-2: 54075657G/C
3-2: 54078172T/C
3-2: 54121073G/A
3-2: 54163388T/A
3-2: 54173152C/T
3-2: 54227419A/G
3-2: 54264566T/G
3-2: 54295870T/A
3-2: 54346169T/C
3-2: 54468145C/T
3-2: 54469418C/T
3-2: 54500602T/C
3-2: 54591884G/A
3-2: 54744859C/T
3-2: 54871151A/G
3-2: 54920255C/T
3-2: 54990191A/C
3-2: 55096341A/G
3-2: 55267409T/G
3-2: 55268710A/G
3-2: 55288331G/C
3-2: 55323716G/C
3-2: 55338139G/A
3-2: 55358824T/G
3-2: 55385245T/C
3-2: 55406858T/A
3-2: 55455190T/C
3-2: 55489335A/C
3-2: 55502717T/C
3-2: 55517792A/T
3-2: 55519847T/C
3-2: 55519870A/C
3-2: 55520129G/T
3-2: 55520320T/G
3-2: 55520447C/T
3-2: 55520896A/G
3-2: 55529464C/A
3-2: 55530825C/G
3-2: 55552688G/T
3-2: 55578494C/T
3-2: 55620404C/A
3-2: 55698930T/C
3-2: 55771123G/T
3-2: 55831827C/A
3-2: 55895318T/A
3-2: 55934158G/T
3-2: 55971947C/T
3-2: 56022119T/C
3-2: 56034529G/T
3-2: 56049369G/A
3-2: 56100638T/C
3-2: 56101235C/T
3-2: 56129717T/G
3-2: 56159098G/T
3-2: 56201348T/C
3-2: 56243277C/G
3-2: 56395186T/A
3-2: 56427920T/C
3-2: 56466226A/T
3-2: 56476459A/T
3-2: 56501924G/A
3-2: 56550294C/T
3-2: 56588717A/C
3-2: 56612288A/G
3-2: 56612850T/A
3-2: 56612933C/T
3-2: 56612973A/G
3-2: 56613928C/T
3-2: 56613952A/G
3-2: 56614943T/C
3-2: 56615972T/C
3-2: 56648935A/G
3-2: 56709914G/A
3-2: 56721002C/T
3-2: 56731248A/C
3-2: 56813352C/G
3-2: 56864528C/T
3-2: 56878040C/T
3-2: 56946245C/T
3-2: 57088908A/G
3-2: 57111703G/A
3-2: 57132467T/A
3-2: 57151539C/G
3-2: 57178269G/T
3-2: 57193754G/A
3-2: 57205850T/C
3-2: 57280822A/T
3-2: 57449233G/A
3-2: 57467169G/A
3-2: 57480451C/T
3-2: 57504861C/G
3-2: 57524575G/A
3-2: 57524727G/A
3-2: 57525904A/G
3-2: 57567502T/C
3-2: 57593448G/T
3-2: 57630597A/G
3-2: 57723247A/G
3-2: 57763903C/T
3-2: 57800374C/T
3-2: 57834945G/A
3-2: 57845168C/T
3-2: 57860275T/C
3-2: 57885507A/G
3-2: 57917578T/C
3-2: 58004570T/A
3-2: 58004928A/G
3-2: 58005036G/A
3-2: 58005204A/T
3-2: 58005317C/A
3-2: 58005365G/A
3-2: 58005438A/G
3-2: 58005597C/T
3-2: 58009543C/G
3-2: 58019606A/G
3-2: 58039779T/C
3-2: 58067411T/C
3-2: 58085972G/A
3-2: 58096579G/A
3-2: 58219134C/T
3-2: 58285406G/A
3-2: 58357897T/C
3-2: 58360857C/T
3-2: 58487557G/T
3-2: 58571493G/C
3-2: 58698865G/A
3-2: 58713257C/T
3-2: 58721420C/T
3-2: 58721454T/C
3-2: 58721532A/T
3-2: 58721934C/T
3-2: 58725339C/G
3-2: 58739295G/A
3-2: 58753983A/G
3-2: 58780268T/C
3-2: 58797022C/A
3-2: 58803976T/C
3-2: 58842720T/C
3-2: 58850268C/G
3-2: 58854322C/T
3-2: 58860440A/G
3-2: 58870631G/T
3-2: 58882611C/A
3-2: 58948937C/T
3-2: 58976161A/T
3-2: 59023445T/.
3-2: 59025953G/A
3-2: 59027375C/T
3-2: 59049634T/C
3-2: 59065584G/T
3-2: 59073032G/A
3-2: 59082236C/A
3-2: 59095505C/T
3-2: 59096703T/C
3-2: 59097545A/T
3-2: 59108843G/A
3-2: 59110153T/C
3-2: 59118708T/A
3-2: 59122444G/A
3-2: 59122735A/G
3-2: 59122766G/C
3-2: 59124955A/G
3-2: 59133827A/G
3-2: 59134326T/A
3-2: 59134365A/C
3-2: 59136483C/A
3-2: 59136556C/A
3-2: 59189572A/T
3-2: 59191186A/T
3-2: 59191462T/C
3-2: 59191470C/T
3-2: 59191484C/T
3-2: 59192459T/C
3-2: 59195857G/A
3-2: 59200905A/G
3-2: 59200930C/G
3-2: 59209808G/T
3-2: 59213694A/G
3-2: 59220213G/A
3-2: 59231237A/C
3-2: 59277642T/G
3-2: 59319782T/A
3-2: 59332760C/T
3-2: 59343242G/A
3-2: 59456106T/C
3-2: 59573190T/G
3-2: 59583560G/A
3-2: 59658078G/A
3-2: 59671274A/T
3-2: 59699177T/A
3-2: 59709823C/T
3-2: 59710966T/A
3-2: 59731319A/T
3-2: 59783351T/C
3-2: 59802332G/T
3-2: 59803940A/T
3-2: 59816767A/G
3-2: 59820661A/G
3-2: 59820669C/G
3-2: 59821161G/C
3-2: 59821223C/T
3-2: 59821319A/T
3-2: 59821461G/A
3-2: 59828980T/C
3-2: 59901882A/T
3-2: 59906554A/T
3-2: 59932905G/C
3-2: 59950050A/G
3-2: 59976667C/T
3-2: 59998999G/T
3-2: 60009378G/A
3-2: 60013602C/T
3-2: 60013627A/G
3-2: 60021873C/T
3-2: 60042406A/G
3-2: 60074667C/T
3-2: 60087443T/G
3-2: 60112498G/T
3-2: 60154801A/C
3-2: 60201457C/T
3-2: 60248842C/T
3-2: 60332033G/A
3-2: 60443160A/C
3-2: 60455580A/G
3-2: 60465993A/G
3-2: 60481647A/G
3-2: 60495018T/C
3-2: 60511574C/T
3-2: 60579756A/G
3-2: 60677208C/T
3-2: 60723305A/C
3-2: 60734760G/A
3-2: 60780572G/A
3-2: 60827303C/G
3-2: 60838294C/T
3-2: 60842472A/T
3-2: 60842486T/C
3-2: 60877097A/T
3-2: 60905703G/A
3-2: 60940187G/A
3-2: 60965939A/G
3-2: 60993143C/T
3-2: 61050690T/C
3-2: 61088593G/A
3-2: 61100059G/A
3-2: 61100203G/T
3-2: 61151583A/G
3-2: 61219165T/C
3-2: 61219321G/A
3-2: 61233186G/C
3-2: 61251948G/C
3-2: 61475786G/A
3-2: 61580895T/A
3-2: 61714650A/G
3-2: 61716640A/C
3-2: 61740102C/T
3-2: 61754750C/A
3-2: 61768021A/G
3-2: 61866065G/A
3-2: 61866175G/A
3-2: 61897064G/A
3-2: 61909024A/T
3-2: 61909728G/A
3-2: 61913906C/T
3-2: 61945363T/A
3-2: 61967832A/C
3-2: 61991630A/T
3-2: 62008008T/C
3-2: 62019314T/C
3-2: 62040494T/A
3-2: 62056274A/G
3-2: 62070839T/C
3-2: 62157468G/A
3-2: 62215711T/A
3-2: 62408291A/C
3-2: 62469833G/C
3-2: 62486011C/T
3-2: 62546431T/C
3-2: 62573011C/A
3-2: 62597034T/C
3-2: 62637694G/T
3-2: 62650702G/T
3-2: 62692719C/T
3-2: 62764018C/T
3-2: 62792123C/T
3-2: 62814132T/C
3-2: 62831924A/G
3-2: 62882983G/A
3-2: 62984117T/A
3-2: 63004475T/A
3-2: 63082111G/A
3-2: 63136548G/A
3-2: 63140361A/T
3-2: 63151867A/G
3-2: 63163166C/G
3-2: 63189890A/G
3-2: 63220628C/T
3-2: 63249987C/T
3-2: 63284731G/C
3-2: 63335289T/C
3-2: 63384214G/A
3-2: 63424406C/A
3-2: 63450746A/C
3-2: 63507258C/T
3-2: 63564571T/C
3-2: 63623091T/C
3-2: 63637139A/T
3-2: 63654466T/C
3-2: 63732483G/T
3-2: 63844926C/T
3-2: 63931792G/A
3-2: 64026501A/T
3-2: 64106425C/T
3-2: 64211561C/T
3-2: 64260663C/T
3-2: 64306836C/T
3-2: 64351931T/C
3-2: 64414416C/A
3-2: 64476762C/A
3-2: 64561744A/T
3-2: 64638371C/A
3-2: 64690998T/C
3-2: 64726946A/G
3-2: 64742674G/T
3-2: 64756431A/T
3-2: 64795227T/A
3-2: 64833893T/A
3-2: 64834267A/G
3-2: 64878867T/C
3-2: 64888199G/A
3-2: 64891462A/G
3-2: 64964071A/G
3-2: 65059665G/A
3-2: 65075705G/C
3-2: 65103673T/A
3-2: 65167123A/G
3-2: 65232764G/T
3-2: 65345922T/A
3-2: 65493740G/A
3-2: 65545301A/C
3-2: 65558427A/C
3-2: 65589070T/C
3-2: 65602144T/C
3-2: 65623665C/A
3-2: 65735604A/C
3-2: 65795209T/C
3-2: 65853211G/A
3-2: 65884578C/G
3-2: 65913795C/G
3-2: 65974270C/G
3-2: 65987834A/G
3-2: 66080536T/C
3-2: 66228552T/A
3-2: 66340975A/C
3-2: 66371750A/G
3-2: 66552578C/A
3-2: 66717588C/T
3-2: 66721835G/A
3-2: 66771522T/G
3-2: 66840149T/C
3-2: 66854725A/G
3-2: 66858989C/G
3-2: 66859093G/A
3-2: 66883454C/T
3-2: 66918960A/C
3-2: 66972461C/T
3-2: 67012823A/T
3-2: 67093165C/G
3-2: 67122876T/C
3-2: 67178660A/G
3-2: 67321509A/G
3-2: 67334437C/A
3-2: 67338881G/A
3-2: 67348950G/T
3-2: 67382550A/G
3-2: 67423825C/T
3-2: 67460746A/G
3-2: 67524915A/T
3-2: 67526722A/T
3-2: 67557908C/A
3-2: 67716210G/T
3-2: 67716963G/T
3-2: 67717616T/C
3-2: 67746593G/A
3-2: 67785723C/T
3-2: 67835004C/T
3-2: 68063763C/T
3-2: 68077856G/A
3-2: 68100647A/T
3-2: 68169578T/C
3-2: 68264171G/A
3-2: 68402250T/C
3-2: 68525310A/T
3-2: 68660676G/A
3-2: 68687949A/T
3-2: 68699359T/A
3-2: 68733507A/T
3-2: 68745417C/T
3-2: 68774485A/G
3-2: 68784614C/T
3-2: 68826310C/T
3-2: 68851645C/T
3-2: 68878016T/C
3-2: 68906512T/A
3-2: 68987870A/G
3-2: 68991296C/A
3-2: 69036339G/A
3-2: 69114403C/T
3-2: 69145510G/C
3-2: 69378201T/G
3-2: 69440929T/G
3-2: 69553942A/G
3-2: 69669102C/A
3-2: 69697426A/T
3-2: 69709071G/A
3-2: 69802663C/T
3-2: 69873649A/G
3-2: 69917471T/C
3-2: 69917573A/C
3-2: 69938751T/C
3-2: 69940496A/G
3-2: 69988231G/T
3-2: 69998724G/A
3-2: 70055202A/T
3-2: 70083782C/A
3-2: 70145094A/G
3-2: 70308820G/A
3-2: 70357027A/G
3-2: 70443565T/A
3-2: 70536377C/A
3-2: 70547985G/A
3-2: 70560831A/T
3-2: 70597324A/C
3-2: 70647392T/C
3-2: 70846102C/A
3-2: 70873865C/T
3-2: 71021050G/A
3-2: 71051384C/T
3-2: 71108718T/C
3-2: 71121159C/T
3-2: 71233783C/T
3-2: 71537412G/A
3-2: 71564548T/G
3-2: 71645095C/T
3-2: 71670348G/A
3-2: 71722758T/A
3-2: 71823572A/G
3-2: 71835661C/T
3-2: 71934753T/C
3-2: 71987155A/G
3-2: 72037529C/T
3-2: 72131068G/A
3-2: 72270322C/T
3-2: 72276231G/A
3-2: 72304630T/A
3-2: 72371102C/T
3-2: 72409661T/A
3-2: 72628489A/G
3-2: 72644949T/G
3-2: 72688360A/G
3-2: 72706919A/G
3-2: 72796012A/G
3-2: 72878865C/T
3-2: 72925345T/A
3-2: 72995767C/T
3-2: 73042382G/A
3-2: 73051633T/C
3-2: 73075488G/A
3-2: 73094536A/C
3-2: 73105092C/T
3-2: 73120065G/A
3-2: 73125927C/T
3-2: 73133684C/G
3-2: 73158741A/T
3-2: 73278387C/T
3-2: 73280312C/T
3-2: 73414565T/A
3-2: 73450019T/G
3-2: 73587678A/G
3-2: 73613641C/T
3-2: 73640463G/A
3-2: 73676756G/T
3-2: 73729354G/A
3-2: 73787195T/A
3-2: 73960765T/C
3-2: 74014431T/C
3-2: 74043684A/T
3-2: 74082221A/T
3-2: 74136390G/A
3-2: 74173353C/G
3-2: 74200777T/C
3-2: 74214522C/T
3-2: 74247199T/G
3-2: 74316127T/G
3-2: 74424386C/T
3-2: 74540582T/A
3-2: 74540683T/C
3-2: 74541581T/A
3-2: 74541813T/C
3-2: 74714818G/A
3-2: 74786605G/A
3-2: 74826881G/A
3-2: 74872101T/G
3-2: 74934171A/G
3-2: 74967807G/C
3-2: 74997380C/T
3-2: 74997461A/T
3-2: 74997484A/G
3-2: 75064102A/T
3-2: 75113613T/C
3-2: 75160009T/C
3-2: 75186549T/C
3-2: 75205998G/A
3-2: 75223111A/T
3-2: 75265826G/A
3-2: 75298003G/A
3-2: 75363193T/G
3-2: 75445681A/G
3-2: 75479804A/T
3-2: 75530992G/A
3-2: 75610369G/C
3-2: 75681410A/G
3-2: 75684831A/C
3-2: 75707746T/C
3-2: 75844375T/G
3-2: 75927312G/T
3-2: 76104149A/G
3-2: 76221080T/A
3-2: 76260938G/T
3-2: 76273906T/A
3-2: 76278929T/C
3-2: 76342548T/A
3-2: 76464263C/G
3-2: 76491176T/C
3-2: 76574518A/G
3-2: 76648954T/A
3-2: 76689444C/T
3-2: 76711245T/C
3-2: 76761761A/G
3-2: 76926706A/C
3-2: 76957579A/T
3-2: 77015193C/A
3-2: 77049885G/A
3-2: 77079277C/T
3-2: 77146645C/T
3-2: 77427206C/A
3-2: 77625662T/C
3-2: 77646674G/A
3-2: 77747256T/C
3-2: 77799404C/T
3-2: 77894723C/A
3-2: 77905290C/T
3-2: 77950082C/G
3-2: 78049481G/A
3-2: 78160112T/A
3-2: 78221793T/C
3-2: 78541463T/C
3-2: 78571941A/G
3-2: 78889320T/C
3-2: 78953817T/A
3-2: 78967933G/C
3-2: 79000160T/A
3-2: 79029571T/C
3-2: 79086149G/A
3-2: 79111203C/T
3-2: 79159228G/T
3-2: 79196551C/G
3-2: 79222870G/A
3-2: 79317055T/A
3-2: 79375720T/G
3-2: 79438942T/A
3-2: 79509403T/C
3-2: 79525219T/G
3-2: 79546929T/A
3-2: 79594545A/G
3-2: 79623155A/C
3-2: 79665218T/A
3-2: 79728598C/T
3-2: 79834690A/G
3-2: 79896664G/T
3-2: 79922587G/T
3-2: 80016296G/A
3-2: 80068611G/A
3-2: 80137288A/G
3-2: 80139069A/G
3-2: 80139567C/A
3-2: 80140516C/T
3-2: 80162116C/A
3-2: 80188910C/T
3-2: 80216563C/T
3-2: 80256506G/A
3-2: 80285642G/T
3-2: 80447338C/T
3-2: 80602437T/A
3-2: 80604879C/G
3-2: 80810622T/C
3-2: 80947285G/C
3-2: 80995037C/T
3-2: 81188500A/G
3-2: 81293170A/C
3-2: 81318440A/G
3-2: 81338207G/T
3-2: 81341802C/G
3-2: 81365613T/C
3-2: 81426525G/A
3-2: 81463142T/C
3-2: 81468662T/A
3-2: 81492637T/A
3-2: 81535709G/A
3-2: 81577243C/T
3-2: 81745569A/G
3-2: 81846337C/T
3-2: 81981906G/A
3-2: 82156297C/T
3-2: 82223620C/A
3-2: 82274728G/A
3-2: 82332399C/T
3-2: 82349265G/T
3-2: 82439788G/A
3-2: 82529362C/G
3-2: 82529832A/G
3-2: 82541711A/G
3-2: 82561686T/C
3-2: 82564469C/T
3-2: 82564495C/T
3-2: 82564511A/T
3-2: 82713291G/A
3-2: 82764655G/A
3-2: 82847676A/G
3-2: 82847850G/A
3-2: 82848004A/T
3-2: 82848380C/A
3-2: 82849548A/G
3-2: 82862034C/T
3-2: 82949452G/A
3-2: 82975917G/A
3-2: 83053824A/T
3-2: 83082813A/T
3-2: 83346417C/T
3-2: 83388494G/T
3-2: 83601278G/A
3-2: 83726257T/C
3-2: 83741765C/T
3-2: 83885950A/G
3-2: 83921379T/C
3-2: 83963680T/C
3-2: 84141835G/T
3-2: 84154744A/G
3-2: 84343892A/G
3-2: 84439385T/C
3-2: 84548807G/T
3-2: 84775576T/A
3-2: 85033263A/T
3-2: 85066187C/T
3-2: 85123351A/G
3-2: 85229718T/G
3-2: 85240185T/A
3-2: 85373339G/A
3-2: 85404060T/C
3-2: 85473556G/T
3-2: 85585203T/C
3-2: 85683657C/G
3-2: 85803406G/A
3-2: 85885720G/A
3-2: 86049277G/A
3-2: 86049494A/T
3-2: 86082452T/C
3-2: 86159020G/A
3-2: 86313258T/A
3-2: 86335238A/G
3-2: 86505813T/C
3-2: 86538118G/A
3-2: 86620794A/C
3-2: 86792517T/C
3-2: 86866201A/G
3-2: 86964457T/C
3-2: 86999113T/C
3-2: 87037250T/A
3-2: 87083326A/T
3-2: 87111342T/A
3-2: 87185302C/T
3-2: 87224490C/T
3-2: 87247544C/G
3-2: 87286609A/G
3-2: 87317333T/G
3-2: 87385910T/C
3-2: 87510301C/T
3-2: 87538532G/C
3-2: 87566848G/T
3-2: 87604298C/T
3-2: 87631968G/A
3-2: 87754110A/G
3-2: 87837484A/T
3-2: 87845604T/C
3-2: 88046716C/T
3-2: 88053332C/A
3-2: 88082306A/C
3-2: 88099314T/A
3-2: 88217056A/G
3-2: 88371628T/A
3-2: 88417912A/T
3-2: 88457481C/A
3-2: 88494865A/G
3-2: 88602247C/T
3-2: 88742927G/A
3-2: 88783760G/A
3-2: 88856106C/G
3-2: 88870662T/C
3-2: 88962715G/C
3-2: 88964299T/C
3-2: 89048007T/C
3-2: 89121824C/A
3-2: 89174361G/A
3-2: 89419886T/G
3-2: 89473820G/C
3-2: 89598181G/T
3-2: 89679382G/A
3-2: 89893729A/G
3-2: 89904874T/A
3-2: 89905413A/C
3-2: 89953641G/A
3-2: 90054526T/C
3-2: 90066978A/T
3-2: 90103840A/T
3-2: 90132357C/T
3-2: 90206295T/A
3-2: 90218090G/T
3-2: 90323965T/C
3-2: 90647584T/C
3-2: 90831151T/C
3-2: 91018265G/T
3-2: 91178827G/T
3-2: 91236150T/A
3-2: 91415254T/G
3-2: 91555737A/G
3-3: 117156A/C
3-3: 1095868G/A
3-3: 1214550C/T
3-3: 1216677G/A
3-3: 1347072A/G
3-3: 1349555A/G
3-3: 1602039T/C
3-3: 1671226A/T
3-3: 1726906T/A
3-3: 1746659T/C
3-3: 1828375A/T
3-3: 1842422A/T
3-3: 2060672A/T
3-3: 2176152A/T
3-3: 2345890T/A
3-3: 2386079T/A
3-3: 2566229C/T
3-3: 2748744G/T
3-3: 2775500T/G
3-3: 2834215A/G
3-3: 3216011A/T
3-3: 3243862G/T
3-3: 3347689A/G
3-3: 3430294T/A
3-3: 3506403A/G
3-3: 3778281C/T
3-3: 4647165A/G
3-3: 4715135T/C
3-3: 5103997T/G
3-3: 5124858C/T
3-3: 5464133T/C
3-3: 5794226T/C
3-3: 5938611T/C
3-3: 5971678G/A
3-3: 6018622C/T
3-3: 6087934G/A
3-3: 6089690G/C
3-3: 6159024A/T
3-3: 6252855T/A
3-3: 6387544A/G
3-3: 6433378A/T
3-3: 6508487A/T
3-3: 6508549T/A
3-3: 6547406T/C
3-3: 6559184C/A
3-3: 6671286G/A
3-3: 7007338A/G
3-3: 7399333C/A
3-3: 7518008T/C
3-3: 7569558T/C
3-3: 7698401T/C
3-3: 7951349C/T
3-3: 8031927C/A
3-3: 8033706G/A
3-3: 8071665G/A
3-3: 8083802A/C
3-3: 8165981A/G
3-3: 8439407T/C
3-3: 8478922A/C
3-3: 8523250C/T
3-3: 8685341T/A
3-3: 8730412T/A
3-3: 8927720G/T
3-3: 8987869G/A
3-3: 9045358C/T
3-3: 9072899C/A
3-3: 9093662T/G
3-3: 9094750C/T
3-3: 9128445A/C
3-3: 9204850C/A
3-3: 9250829C/A
3-3: 9258403C/A
3-3: 9262227A/T
3-3: 9305169G/A
3-3: 9306151G/A
3-3: 9371030T/C
3-3: 9414969G/A
3-3: 9480138C/T
3-3: 9580723A/G
3-3: 9623104C/T
3-3: 9636557A/G
3-3: 9673521C/T
3-3: 9686145C/T
3-3: 9704471T/C
3-3: 9704685A/T
3-3: 9704794T/C
3-3: 9714705G/T
3-3: 9841142G/A
3-3: 9862783C/T
3-3: 9904935A/G
3-3: 9925649T/G
3-3: 9967963T/.
3-3: 9986146G/A
3-3: 9996724C/T
3-3: 10003896G/T
3-3: 10023592G/A
3-3: 10027470C/A
3-3: 10036790A/G
3-3: 10063398G/C
3-3: 10095427C/A
3-3: 10122075C/G
3-3: 10160379G/C
3-3: 10191165C/G
3-3: 10197701C/G
3-3: 10230309G/A
3-3: 10283618C/T
3-3: 10381055C/T
3-3: 10451848T/A
3-3: 10462811T/A
3-3: 10553097G/A
3-3: 10709002A/G
3-3: 10725834C/T
3-3: 10750235C/T
3-3: 10757125T/C
3-3: 10771243T/A
3-3: 10795133T/A
3-3: 10863615T/A
3-3: 11040387A/T
3-3: 11172331G/T
3-3: 11296077T/A
3-3: 11303432A/G
3-3: 11306235T/G
3-3: 11330960T/C
3-3: 11426808G/T
3-3: 11455602A/G
3-3: 11503089G/A
3-3: 11540091T/A
3-3: 11542074T/.
3-3: 11550328G/C
3-3: 11561610A/G
3-3: 11576633T/C
3-3: 11614748A/G
3-3: 11665569G/A
3-3: 11677684C/G
3-3: 11693756C/A
3-3: 11713397T/C
3-3: 11742871A/T
3-3: 11776742G/A
3-3: 11793878C/T
3-3: 11809450T/C
3-3: 11819953G/C
3-3: 11837341C/T
3-3: 11862041G/T
3-3: 11893555C/T
3-3: 11925689C/T
3-3: 11952414C/T
3-3: 12012331C/T
3-3: 12020810C/A
3-3: 12020821C/T
3-3: 12209265C/T
3-3: 12233047C/A
3-3: 12244378T/C
3-3: 12302406A/T
3-3: 12446469A/C
3-3: 12529165A/T
3-3: 12557999G/A
3-3: 12611048C/T
3-3: 12685035A/G
3-3: 12729995T/C
3-3: 12792672C/A
3-3: 12829176G/C
3-3: 12874229A/G
3-3: 12886835C/T
3-3: 12948946G/C
3-3: 12958932G/A
3-3: 12963504A/C
3-3: 12975155A/C
3-3: 12985240G/A
3-3: 12997952G/A
3-3: 13019292T/C
3-3: 13045181C/A
3-3: 13089992A/G
3-3: 13103714A/T
3-3: 13119076T/A
3-3: 13162583T/G
3-3: 13200576C/T
3-3: 13227318C/T
3-3: 13233780T/C
3-3: 13233781G/A
3-3: 13233783C/T
3-3: 13233862T/C
3-3: 13233888G/C
3-3: 13233889T/C
3-3: 13234018A/G
3-3: 13234048A/G
3-3: 13234052T/C
3-3: 13242170C/A
3-3: 13242326C/T
3-3: 13245646A/G
3-3: 13247371G/A
3-3: 13247551C/A
3-3: 13247750C/T
3-3: 13247784G/A
3-3: 13247802C/G
3-3: 13247817A/T
3-3: 13247825G/A
3-3: 13247890C/A
3-3: 13247906G/A
3-3: 13273252G/T
3-3: 13360412G/C
3-3: 13362709T/C
3-3: 13392589A/G
3-3: 13416643A/G
3-3: 13442245T/A
3-3: 13456714C/T
3-3: 13494557G/A
3-3: 13536389C/T
3-3: 13544391T/A
3-3: 13571848T/C
3-3: 13630120T/C
3-3: 13666461T/A
3-3: 13748146A/G
3-3: 13759316G/A
3-3: 13788017G/A
3-3: 13789534A/C
3-3: 13804126T/C
3-3: 13857067G/A
3-3: 13884715G/A
3-3: 13978138T/C
3-3: 14021785A/G
3-3: 14069616G/A
3-3: 14132463C/T
3-3: 14150990A/G
3-3: 14168977C/G
3-3: 14194413A/G
3-3: 14194957G/A
3-3: 14215858A/G
3-3: 14269220T/C
3-3: 14472494T/C
3-3: 14484407T/C
3-3: 14588083C/T
3-3: 14593085T/C
3-3: 14712556A/G
3-3: 14891658C/T
3-3: 14941078G/A
3-3: 14980297C/A
3-3: 15088481A/G
3-3: 15141960T/C
3-3: 15156371G/T
3-3: 15312007T/C
3-3: 15360268G/A
3-3: 15394480C/T
3-3: 15430470A/T
3-3: 15626104A/G
3-3: 15637833A/G
3-3: 15682057T/C
3-3: 15806064A/C
3-3: 15818009A/G
3-3: 15895274A/G
3-3: 15916103C/T
3-3: 16032825G/A
3-3: 16033874A/G
3-3: 16052402C/G
3-3: 16082243T/C
3-3: 16110951C/T
3-3: 16152517T/A
3-3: 16330271A/G
3-3: 16330364G/A
3-3: 16330397A/G
3-3: 16333131G/C
3-3: 16337772T/C
3-3: 16337778C/T
3-3: 16384618G/A
3-3: 16462165A/T
3-3: 16606909T/C
3-3: 16635842C/A
3-3: 16661585G/A
3-3: 16685075G/A
3-3: 16787010C/T
3-3: 16806344C/A
3-3: 16817525A/T
3-3: 16888330C/T
3-3: 16989929T/C
3-3: 17100605A/G
3-3: 17100910A/G
3-3: 17111588A/G
3-3: 17144399T/C
3-3: 17180961C/T
3-3: 17182775T/C
3-3: 17182813A/T
3-3: 17182879T/G
3-3: 17186380G/A
3-3: 17196356A/C
3-3: 17196628C/T
3-3: 17209928C/A
3-3: 17223962G/A
3-3: 17224156C/T
3-3: 17258166C/T
3-3: 17362471C/T
3-3: 17391693T/G
3-3: 17392131C/G
3-3: 17443249G/A
3-3: 17469253C/T
3-3: 17474492A/G
3-3: 17540488C/T
3-3: 17668214G/A
3-3: 17828142A/T
3-3: 17829799T/A
3-3: 17829955T/A
3-3: 17830061T/G
3-3: 17830262T/C
3-3: 17830343G/A
3-3: 17830524C/T
3-3: 17832796C/T
3-3: 17833759G/A
3-3: 17833989G/A
3-3: 17834249C/T
3-3: 17834374C/T
3-3: 17834458G/A
3-3: 17834598T/C
3-3: 17834759G/A
3-3: 17835923A/G
3-3: 17836032T/G
3-3: 17836314A/T
3-3: 17836416T/C
3-3: 17836440A/C
3-3: 17836518G/A
3-3: 17836626T/C
3-3: 17836749A/T
3-3: 17836899A/C
3-3: 17836997T/C
3-3: 17837102G/A
3-3: 17837144C/A
3-3: 17837370A/G
3-3: 17837478C/A
3-3: 17837522T/A
3-3: 17837538T/C
3-3: 17837590G/A
3-3: 17837722G/A
3-3: 17838439G/A
3-3: 17920289C/T
3-3: 17936145G/A
3-3: 17951657A/C
3-3: 17962460T/A
3-3: 17963673G/A
3-3: 17966769C/T
3-3: 17966794C/G
3-3: 17966881G/T
3-3: 17988785C/A
3-3: 18055676T/C
3-3: 18117403C/A
3-3: 18146825G/A
3-3: 18172011C/T
3-3: 18173352T/A
3-3: 18180149A/G
3-3: 18206770C/T
3-3: 18245494T/G
3-3: 18338903A/T
3-3: 18344906C/A
3-3: 18423154C/T
3-3: 18500999A/T
3-3: 18502001G/T
3-3: 18549699T/G
3-3: 18757613C/T
3-3: 18772275A/T
3-3: 18782241C/T
3-3: 18782895G/A
3-3: 18817518T/C
3-3: 18899969G/A
3-3: 18957868G/A
3-3: 18979039C/G
3-3: 19008562T/G
3-3: 19152983G/A
3-3: 19153085T/C
3-3: 19162698T/A
3-3: 19179478C/A
3-3: 19263434C/T
3-3: 19291543C/T
3-3: 19304733G/T
3-3: 19315159T/C
3-3: 19315396A/G
3-3: 19330070G/T
3-3: 19400330T/A
3-3: 19428023T/G
3-3: 19442385A/G
3-3: 19444092A/G
3-3: 19453067C/A
3-3: 19500383C/T
3-3: 19594743G/A
3-3: 19620804A/C
3-3: 19655948T/C
3-3: 19668285G/C
3-3: 19758353A/G
3-3: 19771894A/G
3-3: 19794613A/G
3-3: 19852966C/A
3-3: 19880912G/A
3-3: 19928702A/G
3-3: 20003218T/A
3-3: 20037955A/C
3-3: 20055086A/C
3-3: 20080090C/T
3-3: 20117383T/A
3-3: 20143592G/T
3-3: 20168079A/G
3-3: 20183944G/A
3-3: 20233612G/A
3-3: 20275102C/T
3-3: 20435669G/T
3-3: 20464296G/A
3-3: 20486523C/T
3-3: 20583866A/T
3-3: 20631673C/A
3-3: 20644197A/G
3-3: 20646067T/C
3-3: 20646450T/G
3-3: 20646914G/A
3-3: 20651483C/A
3-3: 20651643C/A
3-3: 20651890G/A
3-3: 20655060C/T
3-3: 20671709A/G
3-3: 20754408G/A
3-3: 20764836G/A
3-3: 20764905G/A
3-3: 20842165T/C
3-3: 20857518G/A
3-3: 20877815G/A
3-3: 20891940T/C
3-3: 20906193T/G
3-3: 20976055C/T
3-3: 21007910G/A
3-3: 21061849T/C
3-3: 21080392G/T
3-3: 21100139A/C
3-3: 21128509G/A
3-3: 21155317T/A
3-3: 21192912G/A
3-3: 21218706T/A
3-3: 21222003C/T
3-3: 21254254C/T
3-3: 21334563G/A
3-3: 21375507G/T
3-3: 21382400C/T
3-3: 21384075G/C
3-3: 21393080G/A
3-3: 21445752G/A
3-3: 21507682A/G
3-3: 21534040T/C
3-3: 21670300C/T
3-3: 21671575C/T
3-3: 21740990G/A
3-3: 21772690G/T
3-3: 21847905T/A
3-3: 21912262T/C
3-3: 21912387A/C
3-3: 21912733C/T
3-3: 21912746A/C
3-3: 21924828C/T
3-3: 21974143G/A
3-3: 21993486A/T
3-3: 22019546A/G
3-3: 22037535G/C
3-3: 22090419A/T
3-3: 22220430G/A
3-3: 22242193C/T
3-3: 22274339A/T
3-3: 22306231G/A
3-3: 22380138A/G
3-3: 22428873C/T
3-3: 22446661T/C
3-3: 22483613C/T
3-3: 22586026T/G
3-3: 22642571G/A
3-3: 22732881T/A
3-3: 22865562A/T
3-3: 22895225G/T
3-3: 22938689C/A
3-3: 22964211A/T
3-3: 23008767G/T
3-3: 23022066T/A
3-3: 23191176C/A
3-3: 23324312A/T
3-3: 23485112G/A
3-3: 23520495T/A
3-3: 23574415G/A
3-3: 23606845G/T
3-3: 23737023A/C
3-3: 23745876A/T
3-3: 23784339A/G
3-3: 23848760C/G
3-3: 23965735A/G
3-3: 24006393C/A
3-3: 24101288T/C
3-3: 24179995T/C
3-3: 24225427C/T
3-3: 24252387G/A
3-3: 24306270A/G
3-3: 24333677A/G
3-3: 24383686T/C
3-3: 24410770G/A
3-3: 24424494A/T
3-3: 24540591G/T
3-3: 24550933A/G
3-3: 24557476T/G
3-3: 24565059T/C
3-3: 24648404A/G
3-3: 24658501T/A
3-3: 24699867G/A
3-3: 24766825T/A
3-3: 24842367G/T
3-3: 24997685G/A
3-3: 25064751G/A
3-3: 25110369A/T
3-3: 25211606T/C
3-3: 25230098A/G
3-3: 25254090G/A
3-3: 25282856C/T
3-3: 25307932G/A
3-3: 25375114A/G
3-3: 25380697G/A
3-3: 25392323G/A
3-3: 25422873T/C
3-3: 25444538C/A
3-3: 25544431G/T
3-3: 25633970A/C
3-3: 25686005C/A
3-3: 25734602G/A
3-3: 25917417T/A
3-3: 25922347C/T
3-3: 25954380T/A
3-3: 25954427C/A
3-3: 25954437G/C
3-3: 25954654C/G
3-3: 25963112A/G
3-3: 25963519T/A
3-3: 25973073T/C
3-3: 25973459C/T
3-3: 25973482G/C
3-3: 25973590G/A
3-3: 25974016C/A
3-3: 25974177G/A
3-3: 25974281A/T
3-3: 25996248C/A
3-3: 26013720T/A
3-3: 26069706A/T
3-3: 26317104C/A
3-3: 26510123T/A
3-3: 26549781G/A
3-3: 26600475A/G
3-3: 26671556C/T
3-3: 26712504T/A
3-3: 26716263A/G
3-3: 26751521C/T
3-3: 26793552T/C
3-3: 26811094T/C
3-3: 26811203C/A
3-3: 26812742G/A
3-3: 26812826T/C
3-3: 26825781C/T
3-3: 26826467T/A
3-3: 26827880G/T
3-3: 26863429G/T
3-3: 26876517T/A
3-3: 26921613T/C
3-3: 26996641A/G
3-3: 27191399G/A
3-3: 27206219G/A
3-3: 27321656C/T
3-3: 27326542A/T
3-3: 27360186A/G
3-3: 27472234T/C
3-3: 27490024T/C
3-3: 27500599C/T
3-3: 27619429T/A
3-3: 27622327A/T
3-3: 27685344A/G
3-3: 27701470A/T
3-3: 27718091C/T
3-3: 27738295G/A
3-3: 27749131T/G
3-3: 27772658A/G
3-3: 27775867T/A
3-3: 27801806G/A
3-3: 27846902A/G
3-3: 27935262A/T
3-3: 27937537G/A
3-3: 27937643T/G
3-3: 27937802T/C
3-3: 27946760A/C
3-3: 27946782T/C
3-3: 27947081G/T
3-3: 27957093C/G
3-3: 27957181G/T
3-3: 27958919G/A
3-3: 28038360A/G
3-3: 28148924T/A
3-3: 28155239G/T
3-3: 28166342C/T
3-3: 28167680A/G
3-3: 28267490C/G
3-3: 28291327G/A
3-3: 28317855G/C
3-3: 28333975C/T
3-3: 28445260G/A
3-3: 28453711A/C
3-3: 28506496A/T
3-3: 28541365T/C
3-3: 28631803G/A
3-3: 28634325G/A
3-3: 28761747C/T
3-3: 28983560C/T
3-3: 28988536A/T
3-3: 29036090C/T
3-3: 29081305G/A
3-3: 29141436C/A
3-3: 29193637C/T
3-3: 29204209C/T
3-3: 29215341A/G
3-3: 29244586C/T
3-3: 29244821C/T
3-3: 29246526T/C
3-3: 29247145G/A
3-3: 29247274T/C
3-3: 29265146T/C
3-3: 29336383A/C
3-3: 29381180T/A
3-3: 29412318G/A
3-3: 29474610G/T
3-3: 29528097T/G
3-3: 29677022T/A
3-3: 29704048G/A
3-3: 29729505A/T
3-3: 29764727G/A
3-3: 29766228C/A
3-3: 29777586T/C
3-3: 29905812A/T
3-3: 30020727G/A
3-3: 30041615C/A
3-3: 30072387A/T
3-3: 30206313A/T
3-3: 30210415G/A
3-3: 30450225T/C
3-3: 30519952T/A
3-3: 30541072A/C
3-3: 30563202A/C
3-3: 30582900T/C
3-3: 30625766C/T
3-3: 30681793C/T
3-3: 30712796T/G
3-3: 30812793T/C
3-3: 30844028C/T
3-3: 30884250C/T
3-3: 30884855G/A
3-3: 30885488C/T
3-3: 30913405T/A
3-3: 30943811C/T
3-3: 30973409C/G
3-3: 30992023T/C
3-3: 31027673C/T
3-3: 31028691C/A
3-3: 31029565G/A
3-3: 31029810G/A
3-3: 31029877T/A
3-3: 31030030C/G
3-3: 31038966T/C
3-3: 31039067T/C
3-3: 31040781A/G
3-3: 31041028T/C
3-3: 31041269C/T
3-3: 31041892G/A
3-3: 31042820C/T
3-3: 31042967A/G
3-3: 31125190C/G
3-3: 31189946G/T
3-3: 31217201C/T
3-3: 31319337T/A
3-3: 31320246G/A
3-3: 31322404T/C
3-3: 31323294G/A
3-3: 31325710C/T
3-3: 31325823G/A
3-3: 31326033C/T
3-3: 31329422C/G
3-3: 31330841G/A
3-3: 31331815A/G
3-3: 31332099A/G
3-3: 31332345G/C
3-3: 31332573C/A
3-3: 31332884A/C
3-3: 31333575C/T
3-3: 31341098A/G
3-3: 31353480C/T
3-3: 31418855T/C
3-3: 31461961A/G
3-3: 31464928A/C
3-3: 31467522C/T
3-3: 31468577A/C
3-3: 31480313G/A
3-3: 31492321C/T
3-3: 31580718C/T
3-3: 31611509G/C
3-3: 31625084C/T
3-3: 31644551A/G
3-3: 31728606A/G
3-3: 31740170C/T
3-3: 31770267G/A
3-3: 31846284A/G
3-3: 32132799T/C
3-3: 32306314A/C
3-3: 32400013T/A
3-3: 32618339T/C
3-3: 32821760G/T
3-3: 33016792G/A
3-3: 33112639T/C
3-3: 33158756A/G
3-3: 33182256G/A
3-3: 33297143T/G
3-3: 33299798A/C
3-3: 33422506G/A
3-3: 33491360A/C
3-3: 33531237C/T
3-3: 33731783A/G
3-3: 33851394G/A
3-3: 33884239A/G
3-3: 33915517T/C
3-3: 33933742T/C
3-3: 33946096T/A
3-3: 33992176G/C
3-3: 34005186A/G
3-3: 34096035T/C
3-3: 34160937C/T
3-3: 34301667T/C
3-3: 34330121C/T
3-3: 34370127C/T
3-3: 34456436T/G
3-3: 34501072C/T
3-3: 34524138T/C
3-3: 34540013G/C
3-3: 34773761T/C
3-3: 34775570G/T
3-3: 34788728C/T
3-3: 34812962C/T
3-3: 34859075C/A
3-3: 34967274C/A
3-3: 34979245T/G
3-3: 35001524C/T
3-3: 35108328A/G
3-3: 35345495A/G
3-3: 35353007C/T
3-3: 35377784G/A
3-3: 35377986C/A
3-3: 35548051G/A
3-3: 35617888A/G
3-3: 35664387C/A
3-3: 35764096G/T
3-3: 35793899T/A
3-3: 35796798G/A
3-3: 35836454A/G
3-3: 35836685A/G
3-3: 35867219A/G
3-3: 35876063A/T
3-3: 35878638T/G
3-3: 35878724T/C
3-3: 35878936T/A
3-3: 35879108A/T
3-3: 35879298A/G
3-3: 35879989T/G
3-3: 35890494A/C
3-3: 36182312G/A
3-3: 36373776A/G
3-3: 36395215G/T
3-3: 36399915C/G
3-3: 36427316T/C
3-3: 36730782C/T
3-3: 36791235T/A
3-3: 36845059G/A
3-3: 36914055T/C
3-3: 36957045C/T
3-3: 36987007T/C
3-3: 37042555A/T
3-3: 37072038G/A
3-3: 37079776A/G
3-3: 37097121A/G
3-3: 37128789C/A
3-3: 37171327T/A
3-3: 37186364G/A
3-3: 37239401G/T
3-3: 37261069T/G
3-3: 37313354T/A
3-3: 37316294G/A
3-3: 37317528T/G
3-3: 37327100T/G
3-3: 37371677C/A
3-3: 37385160A/G
3-3: 37403205A/G
3-3: 37434564G/C
3-3: 37481789G/A
3-3: 37561360G/A
3-3: 37564464T/C
3-3: 37591322C/T
3-3: 37677176T/A
3-3: 37717284C/G
3-3: 37762769A/G
3-3: 37794354C/A
3-3: 37929328T/C
3-3: 37935408T/G
3-3: 37949841G/A
3-3: 37972325A/T
3-3: 38013644A/T
3-3: 38037261G/T
3-3: 38071549T/C
3-3: 38075449C/T
3-3: 38087235A/G
3-3: 38127922C/A
3-3: 38153913T/A
3-3: 38200773T/C
3-3: 38210787T/G
3-3: 38364949A/G
3-3: 38543973A/T
3-3: 38573519C/G
3-3: 38583590C/G
3-3: 38595040A/G
3-3: 38674324T/C
3-3: 38716629G/T
3-3: 38730903C/G
3-3: 38797738A/T
3-3: 38943610A/T
3-3: 39022928A/G
3-3: 39103214A/T
3-3: 39161368A/G
3-3: 39161610T/C
3-3: 39260628C/T
3-3: 39265002T/C
3-3: 39359994G/T
3-3: 39370826G/T
3-3: 39569297A/T
3-3: 39671347G/A
3-3: 39672380A/C
3-3: 39682324G/T
3-3: 39695584A/G
3-3: 39706782A/G
3-3: 39715281G/A
3-3: 39731087A/T
3-3: 39823680A/T
3-3: 39948343C/G
3-3: 40022726T/A
3-3: 40044819C/T
3-3: 40090521C/T
3-3: 40195118A/G
3-3: 40196653T/G
3-3: 40205216T/G
3-3: 40208840G/A
3-3: 40358095G/T
3-3: 40425180T/A
3-3: 40453749C/T
3-3: 40512319T/G
3-3: 40558911A/C
3-3: 40657263C/T
3-3: 40692147G/T
3-3: 40742376G/C
3-3: 40763819T/A
3-3: 40904309G/C
3-3: 41045018T/G
3-3: 41090978C/G
3-3: 41102809C/T
3-3: 41195715G/A
3-3: 41291203T/A
3-3: 41433084G/C
3-3: 41473316G/A
3-3: 41555108T/C
3-3: 41770182G/T
3-3: 41781422G/A
3-3: 41783330G/A
3-3: 41795998A/C
3-3: 41823224C/T
3-3: 41914293C/T
3-3: 41926446T/A
3-3: 41958056A/C
3-3: 42013957A/G
3-3: 42049776A/G
3-3: 42079862C/G
3-3: 42110939C/A
3-3: 42232144C/T
3-3: 42325283G/C
3-3: 42383683A/G
3-3: 42433409T/C
3-3: 42524377G/A
3-3: 42554863G/A
3-3: 42690032A/C
3-3: 42702285A/G
3-3: 42770956C/T
3-3: 42856939G/A
3-3: 42907489T/C
3-3: 42925127C/A
3-3: 42936302G/A
3-3: 42942954A/C
3-3: 42943264A/G
3-3: 42943266G/T
3-3: 42943274A/G
3-3: 42944126C/T
3-3: 42951223A/G
3-3: 42963140C/G
3-3: 42993158T/A
3-3: 43017129T/C
3-3: 43027263T/C
3-3: 43124486A/G
3-3: 43148886T/C
3-3: 43221364A/G
3-3: 43254210A/C
3-3: 43276667C/G
3-3: 43332581G/A
3-3: 43361564G/A
3-3: 43379821A/G
3-3: 43412441G/A
3-3: 43420084C/T
3-3: 43422974A/T
3-3: 43423088G/T
3-3: 43434103A/G
3-3: 43435534C/G
3-3: 43500215G/A
3-3: 43529089C/T
3-3: 43542493T/C
3-3: 43555405C/T
3-3: 43573650C/A
3-3: 43591641G/T
3-3: 43591734T/G
3-3: 43604803A/G
3-3: 43610554T/C
3-3: 43616796G/T
3-3: 43669939G/A
3-3: 43731232C/T
3-3: 43757073T/C
3-3: 43825576T/A
3-3: 43861350A/G
3-3: 43885597T/C
3-3: 43893209C/T
3-3: 43895993A/C
3-3: 43898121G/A
3-3: 43901469A/G
3-3: 43915739A/G
3-3: 43926661A/G
3-3: 43960995A/G
3-3: 44011691C/T
3-3: 44055769A/G
3-3: 44135117A/T
3-3: 44155523G/C
3-3: 44159034C/A
3-3: 44159089A/G
3-3: 44159141T/C
3-3: 44160327C/T
3-3: 44160439G/A
3-3: 44160548G/A
3-3: 44160708A/T
3-3: 44160812G/C
3-3: 44161112T/A
3-3: 44161194A/T
3-3: 44161412G/T
3-3: 44165535C/A
3-3: 44175606G/T
3-3: 44209596C/T
3-3: 44260758G/.
3-3: 44266761A/T
3-3: 44338592G/A
3-3: 44392049C/T
3-3: 44430672T/C
3-3: 44442548T/C
3-3: 44502166G/A
3-3: 44531181G/T
3-3: 44557980A/T
3-3: 44587762C/T
3-3: 44614353C/A
3-3: 44617419C/T
3-3: 44632817G/A
3-3: 44656920G/A
3-3: 44661405A/G
3-3: 44689517A/G
3-3: 44740027G/A
3-3: 44852356T/A
3-3: 44866366T/C
3-3: 44898634G/C
3-3: 44933000G/A
3-3: 44993168A/G
3-3: 44993201G/T
3-3: 45002279A/G
3-3: 45002980C/A
3-3: 45003099C/A
3-3: 45003234T/C
3-3: 45004165A/G
3-3: 45014357C/T
3-3: 45021257C/T
3-3: 45061650G/C
3-3: 45129870A/T
3-3: 45156729T/C
3-3: 45198943G/A
3-3: 45237993C/T
3-3: 45286050C/T
3-3: 45359914C/T
3-3: 45438151G/A
3-3: 45457366T/C
3-3: 45467921C/T
3-3: 45527808A/C
3-3: 45530576T/C
3-3: 45551645G/A
3-3: 45568510T/A
3-3: 45591979G/A
3-3: 45602821A/G
3-3: 45614471C/T
3-3: 45618742G/T
3-3: 45650263G/A
3-3: 45840490C/T
3-3: 45846441A/G
3-3: 45851951A/T
3-3: 45872613A/G
3-3: 45945903G/T
3-3: 45978844A/G
3-3: 45998045C/T
3-3: 46147324C/G
3-3: 46169715A/C
3-3: 46186004G/A
3-3: 46225184T/C
3-3: 46236523G/A
3-3: 46282639C/A
3-3: 46354237C/T
3-3: 46426322C/T
3-3: 46450495C/T
3-3: 46451815T/C
3-3: 46486223T/A
3-3: 46487299C/T
3-3: 46537876G/A
3-3: 46542476G/A
3-3: 46551071C/G
3-3: 46575935G/T
3-3: 46596212G/A
3-3: 46694151C/T
3-3: 46737230C/T
3-3: 46779309C/T
3-3: 46819048G/C
3-3: 46853176T/G
3-3: 46942277A/G
3-3: 46970294A/C
3-3: 46971035C/A
3-3: 46971214G/A
3-3: 46971228G/A
3-3: 47003921A/G
3-3: 47014478G/A
3-3: 47036106T/.
3-3: 47041002T/C
3-3: 47052151G/A
3-3: 47067203G/T
3-3: 47081584C/A
3-3: 47092095T/C
3-3: 47121675T/C
3-3: 47169292T/A
3-3: 47207695G/A
3-3: 47213389C/T
3-3: 47214266T/A
3-3: 47233101A/G
3-3: 47245249G/C
3-3: 47245799C/T
3-3: 47305346G/A
3-3: 47337129T/G
3-3: 47349244A/G
3-3: 47369988T/C
3-3: 47377960T/C
3-3: 47378370G/A
3-3: 47404775A/G
3-3: 47437230C/A
3-3: 47467569A/G
3-3: 47478829T/C
3-3: 47488870A/G
3-3: 47500370A/G
3-3: 47608070T/C
3-3: 47620565T/A
3-3: 47653709G/A
3-3: 47654309T/A
3-3: 47656279C/T
3-3: 47689190T/C
3-3: 47689555G/A
3-3: 47702740G/A
3-3: 47808695T/C
3-3: 47895771A/C
3-3: 47912383C/T
3-3: 47918520G/A
3-3: 48005735A/T
3-3: 48063271A/G
3-3: 48081389C/T
3-3: 48126566C/T
3-3: 48146974C/T
3-3: 48147076T/A
3-3: 48149539G/C
3-3: 48149908C/A
3-3: 48150094T/A
3-3: 48150156A/T
3-3: 48150250C/T
3-3: 48160209A/T
3-3: 48168225A/G
3-3: 48168643A/G
3-3: 48170025G/A
3-3: 48172014T/A
3-3: 48172101G/A
3-3: 48172103C/T
3-3: 48172208A/T
3-3: 48172257T/.
3-3: 48185990T/G
3-3: 48245963A/G
3-3: 48249369G/A
3-3: 48256418G/A
3-3: 48257924T/C
3-3: 48258990A/C
3-3: 48259018C/A
3-3: 48268685A/G
3-3: 48328607C/G
3-3: 48335484T/G
3-3: 48336419T/C
3-3: 48348203A/G
3-3: 48392705G/A
3-3: 48395053C/T
3-3: 48442205T/C
3-3: 48442596A/G
3-3: 48557213C/A
3-3: 48572508C/T
3-3: 48598614A/G
3-3: 48635026C/T
3-3: 48638222G/A
3-3: 48737611T/C
3-3: 48755867T/C
3-3: 48756028C/T
3-3: 48757820A/G
3-3: 48761205T/C
3-3: 48761521G/A
3-3: 48762699T/G
3-3: 48762956G/A
3-3: 48771299C/T
3-3: 48790877C/A
3-3: 48826994A/G
3-3: 48878399T/C
3-3: 48925441A/T
3-3: 48991760C/T
3-3: 49048074T/C
3-3: 49097033C/T
3-3: 49097462G/T
3-3: 49097485T/C
3-3: 49126425A/T
3-3: 49157743A/G
3-3: 49209133T/C
3-3: 49228323A/G
3-3: 49301545C/T
3-3: 49323035G/T
3-3: 49326605T/.
3-3: 49329663C/T
3-3: 49329774A/T
3-3: 49330104G/A
3-3: 49330779C/T
3-3: 49332079A/G
3-3: 49336650C/A
3-3: 49378650A/G
3-3: 49434979A/T
3-3: 49435093A/G
3-3: 49506768C/G
3-3: 49590953T/G
3-3: 49669023G/A
3-3: 49698535G/T
3-3: 49761178G/A
3-3: 49835828G/A
3-3: 49852397T/C
3-3: 49978231C/T
3-3: 49995285C/T
3-3: 50052028A/G
3-3: 50062902A/G
3-3: 50085987A/G
3-3: 50096282G/A
3-3: 50119116G/A
3-3: 50146963C/T
3-3: 50225043G/C
3-3: 50317370T/G
3-3: 50366823G/A
3-3: 50410614C/T
3-3: 50437989G/A
3-3: 50601685T/C
3-3: 50627370C/A
3-3: 50687421C/T
3-3: 50782535T/A
3-3: 50812983G/A
3-3: 50885399G/A
3-3: 50905704G/T
3-3: 50944355A/G
3-3: 50956278T/A
3-3: 51001181G/A
3-3: 51001942G/C
3-3: 51062623A/G
3-3: 51083094G/T
3-3: 51160935A/T
3-3: 51166332G/A
3-3: 51182491C/T
3-3: 51286970G/A
3-3: 51307308C/T
3-3: 51319158G/A
3-3: 51359989C/A
3-3: 51378391A/G
3-3: 51380088T/C
3-3: 51427265T/C
3-3: 51428904C/T
3-3: 51429299T/C
3-3: 51435594G/T
3-3: 51435657A/C
3-3: 51438622C/T
3-3: 51470092A/G
3-3: 51519469G/A
3-3: 51537649G/A
3-3: 51628426T/A
3-3: 51669056T/G
3-3: 51707294T/C
3-3: 51725044T/A
3-3: 51796858C/T
3-3: 51921293G/A
3-3: 52028622G/A
3-3: 52069242C/T
3-3: 52080101G/C
3-3: 52093420T/A
3-3: 52213902T/C
3-3: 52258063C/A
3-3: 52322951A/T
3-3: 52335518T/A
3-3: 52348055G/T
3-3: 52360782A/T
3-3: 52377510A/T
3-3: 52474838C/T
3-3: 52478157T/G
3-3: 52478232C/T
3-3: 52478236T/C
3-3: 52478377A/G
3-3: 52478772C/A
3-3: 52487042T/A
3-3: 52531008A/T
3-3: 52624491G/A
3-3: 52663054G/A
3-3: 52689691A/G
3-3: 52750751A/T
3-3: 52783675C/T
3-3: 52789662A/G
3-3: 52798765A/G
3-3: 52809229G/C
3-3: 52850794A/C
3-3: 52882727T/C
3-3: 52902019G/A
3-3: 52902886T/A
3-3: 52941361C/A
3-3: 53029612G/T
3-3: 53035941A/T
3-3: 53053896G/A
3-3: 53066303T/A
3-3: 53066539A/G
3-3: 53076735G/A
3-3: 53096323G/A
3-3: 53125242T/G
3-3: 53142544T/G
3-3: 53345236A/C
3-3: 53373856G/A
3-3: 53389809T/C
3-3: 53514665G/T
3-3: 53545859A/G
3-3: 53559600G/A
3-3: 53576107G/C
3-3: 53576515A/G
3-3: 53615210T/A
3-3: 53634072G/.
3-3: 53642064C/T
3-3: 53676738T/A
3-3: 53721292C/A
3-3: 53734739T/C
3-3: 53747198T/C
3-3: 53795283G/T
3-3: 53850733A/T
3-3: 53851260T/C
3-3: 53865385A/T
3-3: 53868911C/T
3-3: 53886459A/C
3-3: 53904763A/T
3-3: 53921547G/A
3-3: 53950404G/T
3-3: 54005208C/A
3-3: 54041846G/T
3-3: 54053362C/T
3-3: 54072309C/T
3-3: 54080080C/T
3-3: 54080684T/A
3-3: 54082341A/T
3-3: 54099021T/C
3-3: 54117739T/A
3-3: 54132787A/T
3-3: 54148057T/C
3-3: 54154200A/G
3-3: 54157082A/C
3-3: 54157107C/G
3-3: 54171495G/T
3-3: 54199697C/T
3-3: 54230282T/C
3-3: 54263397A/G
3-3: 54268053G/A
3-3: 54276775G/A
3-3: 54299432T/C
3-3: 54312831A/G
3-3: 54359099T/G
3-3: 54387843C/T
3-3: 54400712G/T
3-3: 54400714A/T
3-3: 54413524C/T
3-3: 54471491G/A
3-3: 54475502T/C
3-3: 54475603T/A
3-3: 54493934T/A
3-3: 54498336A/C
3-3: 54498446T/C
3-3: 54498917T/A
3-3: 54498994C/T
3-3: 54499716C/T
3-3: 54538798G/A
3-3: 54559056T/C
3-3: 54585127T/C
3-3: 54632508C/T
3-3: 54632892C/T
3-3: 54633381C/T
3-3: 54633531C/T
3-3: 54633676A/G
3-3: 54633680A/C
3-3: 54634010A/T
3-3: 54637967C/T
3-3: 54645894T/C
3-3: 54657128A/G
3-3: 54697547G/A
3-3: 54759289C/T
3-3: 54794288T/G
3-3: 54822062T/A
3-3: 54859765T/A
3-3: 54876852C/T
3-3: 54877104C/T
3-3: 54877339T/C
3-3: 54877484T/A
3-3: 54906508A/G
3-3: 55026269G/A
3-3: 55065127A/G
3-3: 55093927C/T
3-3: 55110854G/A
3-3: 55141759T/G
3-3: 55198489G/A
3-3: 55249570C/T
3-3: 55259128A/T
3-3: 55259188T/A
3-3: 55259320G/A
3-3: 55259425T/C
3-3: 55259682T/A
3-3: 55259701G/A
3-3: 55259743G/A
3-3: 55298708A/T
3-3: 55311077T/A
3-3: 55327465G/C
3-3: 55328362C/T
3-3: 55341090C/T
3-3: 55342590T/G
3-3: 55342831G/T
3-3: 55342907T/A
3-3: 55350531T/C
3-3: 55351161A/G
3-3: 55351182G/A
3-3: 55410190G/C
3-3: 55452202C/A
3-3: 55482467G/A
3-3: 55508441T/C
3-3: 55545387C/T
3-3: 55559652T/C
3-3: 55572292C/T
3-3: 55604186T/C
3-3: 55605916A/G
3-3: 55606676G/A
3-3: 55607056G/A
3-3: 55607326A/C
3-3: 55607702T/A
3-3: 55608837T/C
3-3: 55608862G/A
3-3: 55615180A/T
3-3: 55653584C/A
3-3: 55681853T/C
3-3: 55717655T/G
3-3: 55757051T/C
3-3: 55777740C/T
3-3: 55790947C/G
3-3: 55792146T/C
3-3: 55792637G/A
3-3: 55792640G/A
3-3: 55792740C/T
3-3: 55801264C/T
3-3: 55906265C/T
3-3: 55945331T/G
3-3: 56025597C/T
3-3: 56032250A/T
3-3: 56040364C/G
3-3: 56082294A/T
3-3: 56098463T/G
3-3: 56175095C/T
3-3: 56213007A/G
3-3: 56229101A/G
3-3: 56242982C/A
3-3: 56290730G/T
3-3: 56301108T/A
3-3: 56377032T/A
3-3: 56377576A/G
3-3: 56396882T/C
3-3: 56397233G/T
3-3: 56445978C/T
3-3: 56466651T/A
3-3: 56477246G/A
3-3: 56487035T/G
3-3: 56542025G/A
3-3: 56593902A/G
3-3: 56609531T/C
3-3: 56652659T/A
3-3: 56695648T/C
3-3: 56719748C/A
3-3: 56731627C/T
3-3: 56753326T/C
3-3: 56858730G/A
3-3: 56897678T/C
3-3: 56962765A/G
3-3: 57023126C/T
3-3: 57037138C/T
3-3: 57126318G/T
3-3: 57129754C/T
3-3: 57148613G/A
3-3: 57158791G/C
3-3: 57203619A/G
3-3: 57220288T/C
3-3: 57220977G/A
3-3: 57251570T/G
3-3: 57275899C/A
3-3: 57291171A/G
3-3: 57296589G/A
3-3: 57371697G/A
3-3: 57392192A/C
3-3: 57437206T/C
3-3: 57458875A/T
3-3: 57489264A/C
3-3: 57648694A/T
3-3: 57663648T/C
3-3: 57798737C/T
3-3: 57834934A/G
3-3: 57866781T/G
3-3: 57918656T/A
3-3: 57987131A/C
3-3: 58041549T/C
3-3: 58079008T/A
3-3: 58122075A/T
3-3: 58140855T/A
3-3: 58194929T/A
3-3: 58207021A/G
3-3: 58231302G/A
3-3: 58234803T/C
3-3: 58247909C/T
3-3: 58270885A/G
3-3: 58320667A/T
3-3: 58365755T/A
3-3: 58403555G/A
3-3: 58470767A/G
3-3: 58499068G/A
3-3: 58539034C/T
3-3: 58577377A/T
3-3: 58625522T/C
3-3: 58635788A/G
3-3: 58651811A/T
3-3: 58769061C/T
3-3: 58806304T/C
3-3: 58844244A/C
3-3: 58870321G/A
3-3: 58915129T/G
3-3: 59022351A/C
3-3: 59088265G/A
3-3: 59101526C/T
3-3: 59112637G/A
3-3: 59112780A/G
3-3: 59141712T/C
3-3: 59201957C/T
3-3: 59273926G/A
3-3: 59314847A/T
3-3: 59369485C/T
3-3: 59424493G/A
3-3: 59502199T/C
3-3: 59553821T/C
3-3: 59562262G/A
3-3: 59563769C/A
3-3: 59563803C/T
3-3: 59606085G/T
3-3: 59627523A/G
3-3: 59639772G/A
3-3: 59654907A/G
3-3: 59656266C/A
3-3: 59656980A/G
3-3: 59658002G/A
3-3: 59658318T/C
3-3: 59666175A/G
3-3: 59673570C/T
3-3: 59721856C/T
3-3: 59801224T/C
3-3: 59845075A/G
3-3: 59878433T/C
3-3: 59892771C/A
3-3: 59928976A/G
3-3: 59956821G/A
3-3: 59963561T/C
3-3: 59968423A/T
3-3: 59999734T/A
3-3: 59999787A/C
3-3: 60184683G/A
3-3: 60195845T/C
3-3: 60206262G/T
3-3: 60212646G/A
3-3: 60248465A/G
3-3: 60274036T/C
3-3: 60303402C/A
3-3: 60349358A/G
3-3: 60370283A/T
3-3: 60381761T/C
3-3: 60410716G/A
3-3: 60448076C/T
3-3: 60477550C/G
3-3: 60498405G/A
3-3: 60563209G/T
3-3: 60662583T/A
3-3: 60780940A/C
3-3: 60843828C/G
3-3: 60872199A/T
3-3: 60901618T/C
3-3: 60914502C/T
3-3: 60970796G/A
3-3: 60998530G/A
3-3: 61031705A/G
3-3: 61136693T/G
3-3: 61152869G/A
3-3: 61198180G/T
3-3: 61263615C/T
3-3: 61290137G/T
3-3: 61408905G/A
3-3: 61479193A/G
3-3: 61493063T/C
3-3: 61574035C/T
3-3: 61612411C/T
3-3: 61655545T/A
3-3: 61753514A/G
3-3: 61884712G/A
3-3: 61906810C/T
3-3: 61943531A/T
3-3: 61956504A/T
3-3: 61987779G/T
3-3: 62014268C/A
3-3: 62040767G/T
3-3: 62093438G/C
3-3: 62127001G/A
3-3: 62187088G/A
3-3: 62214193T/C
3-3: 62296421T/C
3-3: 62349107G/A
3-3: 62372143G/A
3-3: 62461483T/G
3-3: 62474848G/A
3-3: 62485355C/T
3-3: 62496072T/C
3-3: 62521038C/T
3-3: 62538932G/T
3-3: 62563810T/C
3-3: 62589234T/C
3-3: 62658997A/G
3-3: 62733266C/A
3-3: 62765054G/A
3-3: 62856263A/G
3-3: 62867422A/T
3-3: 62910492T/A
3-3: 62940399A/G
3-3: 62963755G/A
3-3: 62992730G/A
3-3: 63005843T/C
3-3: 63045929T/G
3-3: 63077134G/A
3-3: 63090836C/A
3-3: 63091882A/G
3-3: 63107936A/G
3-3: 63119952T/C
3-3: 63154906A/G
3-3: 63188118G/A
3-3: 63189340T/G
3-3: 63210865C/A
3-3: 63231451C/T
3-3: 63273779C/T
3-3: 63310281A/T
3-3: 63330220C/T
3-3: 63346240T/C
3-3: 63348691G/A
3-3: 63349487A/G
3-3: 63384873G/A
3-3: 63392704A/T
3-3: 63392952A/G
3-3: 63393319G/C
3-3: 63407987A/G
3-3: 63418904C/T
3-3: 63434781C/A
3-3: 63475060A/T
3-3: 63503325T/A
3-3: 63545372A/G
3-3: 63596589A/G
3-3: 63629954T/C
3-3: 63669572C/T
3-3: 63747273A/T
3-3: 63782913C/T
3-3: 63837025C/T
3-3: 63840613G/C
3-3: 64030445T/G
3-3: 64108751C/T
3-3: 64332521T/C
3-3: 64351643T/C
3-3: 64421235A/G
3-3: 64494290C/T
3-3: 64551084G/T
3-3: 64600012G/A
3-3: 64630950G/A
3-3: 64721746G/C
3-3: 64757196C/T
3-3: 64782344C/A
3-3: 64810643A/G
3-3: 64880052T/C
3-3: 65006406T/C
3-3: 65036562A/G
3-3: 65161843T/G
3-3: 65167337C/T
3-3: 65251059G/A
3-3: 65305597G/A
3-3: 65343601A/G
3-3: 65377388C/T
3-3: 65377492C/T
3-3: 65377603A/T
3-3: 65487153G/T
3-3: 65500453G/A
3-3: 65500854A/G
3-3: 65501048C/T
3-3: 65501235G/A
3-3: 65547786T/G
3-3: 65558660C/T
3-3: 65715458T/C
3-3: 65729747A/G
3-3: 65902520G/A
3-3: 65991804A/G
3-3: 66043908A/C
3-3: 66103595C/T
3-3: 66150544T/C
3-3: 66242975T/G
3-3: 66319469T/A
3-3: 66334612A/G
3-3: 66428298A/G
3-3: 66436510A/T
3-3: 66528939A/T
3-3: 66542737A/G
3-3: 66587043G/A
3-3: 66587533C/T
3-3: 66587550A/G
3-3: 66587610C/T
3-3: 66587768C/T
3-3: 66614028C/T
3-3: 66736924G/A
3-3: 66751275T/C
3-3: 66763694C/G
3-3: 66804759T/C
3-3: 66807127A/C
3-3: 66843604G/T
3-3: 66916026A/G
3-3: 66995340A/G
3-3: 67015061G/A
3-3: 67027977C/T
3-3: 67031890T/C
3-3: 67031915A/C
3-3: 67090161C/T
3-3: 67142933C/G
3-3: 67190221A/G
3-3: 67213891G/A
3-3: 67236987A/T
3-3: 67277563A/T
3-3: 67292688C/T
3-3: 67308178C/A
3-3: 67372902A/C
3-3: 67406807A/G
3-3: 67436899C/A
3-3: 67464332A/T
3-3: 67561092G/A
3-3: 67649022C/A
3-3: 67695440A/G
3-3: 67722356G/T
3-3: 67806623G/C
3-3: 67902042G/T
3-3: 67952716A/G
3-3: 67983791A/G
3-3: 67990759A/G
3-3: 68027556A/G
3-3: 68064357G/C
3-3: 68069893G/A
3-3: 68070246G/A
3-3: 68094649A/T
3-3: 68149339T/G
3-3: 68277181G/A
3-3: 68292909T/C
3-3: 68307312A/T
3-3: 68359936A/C
3-3: 68384156T/A
3-3: 68493213T/C
3-3: 68519693C/T
3-3: 68548736C/T
3-3: 68586790C/T
3-3: 68728299A/G
3-3: 68733212C/G
3-3: 68781251T/C
3-3: 68810453G/A
3-3: 68810582C/A
3-3: 68873294G/A
3-3: 68973984A/G
3-3: 69002106A/G
3-3: 69163867A/C
3-3: 69197320T/G
3-3: 69497234C/T
3-3: 69507319G/A
3-3: 69548615C/T
3-3: 69603275A/G
3-3: 69751729A/G
3-3: 69936930T/C
3-3: 69997403T/G
3-3: 70205699T/C
3-3: 70243128C/G
3-3: 70335393T/C
3-3: 70376693C/T
3-3: 70545850T/C
3-3: 70545852A/C
3-3: 70546321G/A
3-3: 70551276C/A
3-3: 70597466T/C
3-3: 70618142T/C
3-3: 70653615C/T
3-3: 70713317C/T
3-3: 70777450A/G
3-3: 70830118G/A
3-3: 70926639C/T
3-3: 71009796C/T
3-3: 71070996C/T
3-3: 71081263C/A
3-3: 71095568G/A
3-3: 71145677C/A
3-3: 71259520A/T
3-3: 71315355G/T
3-3: 71356099G/A
3-3: 71419451C/T
3-3: 71488631G/A
3-3: 71695094T/A
3-3: 71728005G/C
3-3: 71809763G/C
3-3: 71851925C/G
3-3: 71908272T/C
3-3: 72139737C/T
3-3: 72140749G/A
3-3: 72140877T/C
3-3: 72140975A/G
3-3: 72152406T/A
3-3: 72172345C/T
3-3: 72351606A/G
3-3: 72382654T/A
3-3: 72434658A/C
3-3: 72526010G/C
3-3: 72555287T/A
3-3: 72587957T/G
3-3: 72600306G/A
3-3: 72609678G/A
3-3: 72659684C/T
3-3: 72669786A/T
3-3: 72696262T/C
3-3: 72737882T/C
3-3: 72756949A/G
3-3: 72788992C/T
3-3: 72793351T/C
3-3: 72798526A/T
3-3: 72815935A/C
3-3: 72853236A/G
3-3: 72877495A/C
3-3: 72897367C/T
3-3: 72921615A/G
3-3: 72960228T/C
3-3: 72982725T/C
3-3: 73005364A/T
3-3: 73005673C/T
3-3: 73005854G/A
3-3: 73005883T/C
3-3: 73005992G/A
3-3: 73007265T/C
3-3: 73024253A/G
3-3: 73024429A/T
3-3: 73035332A/G
3-3: 73036298G/A
3-3: 73043589C/T
3-3: 73050747T/C
3-3: 73076302A/G
3-3: 73102429T/.
3-3: 73117754T/G
3-3: 73137251A/G
3-3: 73149457T/G
3-3: 73180774C/T
3-3: 73237345C/T
3-3: 73266888T/C
3-3: 73374842G/T
3-3: 73439627G/A
3-3: 73477715C/G
3-3: 73509520G/T
3-3: 73533526A/T
3-3: 73626304G/A
3-3: 73652830T/A
3-3: 73691801C/A
3-3: 73733264G/A
3-3: 73978903T/A
3-3: 74070625T/C
3-3: 74173502G/T
3-3: 74209222C/T
3-3: 74210357T/C
3-3: 74210480C/T
3-3: 74234619G/A
3-3: 74244624C/T
3-3: 74246692G/C
3-3: 74247369G/A
3-3: 74277330C/T
3-3: 74324135C/G
3-3: 74335767G/A
3-3: 74351990T/C
3-3: 74415245G/A
3-3: 74529535T/A
3-3: 74602281A/T
3-3: 74696060C/T
3-3: 74762578A/G
3-3: 74801776T/C
3-3: 74801784C/T
3-3: 74847103T/C
3-3: 74885745G/T
3-3: 75160390A/G
3-3: 75201764C/T
3-3: 75338242T/A
3-3: 75431667A/C
3-3: 75457687T/C
3-3: 75596122C/T
3-3: 75687104G/A
3-3: 75811642A/T
3-3: 75928539A/G
3-3: 75931238C/A
3-3: 75953648T/C
3-3: 76064315C/T
3-3: 76101012A/G
3-3: 76145867A/G
3-3: 76188300A/G
3-3: 76226514G/A
3-3: 76289690T/C
3-3: 76354316A/G
3-3: 76399462T/A
3-3: 76428990A/T
3-3: 76446903A/G
3-3: 76547242G/T
3-3: 76790277A/T
3-3: 76869186G/A
3-3: 76940678A/G
3-3: 77108324A/G
3-3: 77285030T/A
3-3: 77353078C/T
3-3: 77413051C/T
3-3: 77449828G/A
3-3: 77489990T/C
3-3: 77525680G/A
3-3: 77552134T/G
3-3: 77585962G/C
3-3: 77586557T/G
3-3: 77586567C/A
3-3: 77659314G/T
3-3: 77683917A/C
3-3: 77729631C/T
3-3: 77757556G/C
3-3: 77792267T/C
3-3: 77847506C/T
3-3: 77890153A/G
3-3: 77927600T/C
3-3: 77957283T/A
3-3: 77979328A/G
3-3: 78013829A/C
3-3: 78034553A/G
3-3: 78039498C/A
3-3: 78039893A/C
3-3: 78084898C/T
3-3: 78122975C/T
3-3: 78148941C/T
3-3: 78167923T/A
3-3: 78178358G/A
3-3: 78219413G/A
3-3: 78258430C/A
3-3: 78289067T/C
3-3: 78349860C/T
3-3: 78362699A/G
3-3: 78388250A/G
3-3: 78415497T/C
3-3: 78459446A/G
3-3: 78474271C/T
3-3: 78524002G/T
3-3: 78578218C/T
3-3: 78623680T/G
3-3: 78660231T/A
3-3: 78704060C/T
3-3: 78729538C/G
3-3: 78763902C/G
3-3: 78791756G/A
3-3: 78921092C/A
3-3: 79041115C/T
3-3: 79092676G/A
3-3: 79264794G/A
3-3: 79394893A/G
3-3: 79395237T/A
3-3: 79472135A/T
3-3: 79528409C/T
3-3: 79667694G/A
3-3: 79727785T/C
3-3: 79746737C/T
3-3: 79789418C/T
3-3: 79804964T/A
3-3: 79822183C/T
3-3: 79973535A/G
3-3: 80007836T/C
3-3: 80038281C/T
3-3: 80081680C/A
3-3: 80186494C/G
3-3: 80242596G/T
3-3: 80277131G/A
3-3: 80307968A/G
3-3: 80372428T/C
3-3: 80423009G/A
3-3: 80451882G/T
3-3: 80518162T/G
3-3: 80566715C/T
3-3: 80625484C/A
3-3: 80903978T/C
3-3: 80979591C/T
3-3: 81049751T/A
3-3: 81144387G/A
3-3: 81294283A/G
3-3: 81589708T/C
3-3: 81597122A/G
3-3: 81653782G/A
3-3: 81713927A/G
3-3: 81748314G/C
3-3: 81918223G/A
3-3: 82154548G/A
3-3: 82218332C/T
3-3: 82279327C/A
3-3: 82389719C/T
3-3: 82444489T/C
3-3: 82494899C/A
3-3: 82621519T/C
3-3: 82780257C/T
3-3: 82798155T/C
3-3: 82817566A/T
3-3: 82843524C/T
3-3: 82958421G/A
3-3: 83026575T/G
3-3: 83152615C/T
3-3: 83221803C/T
3-3: 83310505C/T
3-3: 83409982C/T
3-3: 83663050G/A
3-3: 83704728C/T
3-3: 83886488C/T
3-3: 84042659G/T
3-3: 84138467G/A
3-3: 84155942A/T
3-3: 84180534A/T
3-3: 84250859G/T
3-3: 84281879G/A
3-3: 84311289T/C
3-3: 84370873T/C
3-3: 84509845C/T
3-3: 84538589T/C
3-3: 84616998C/T
3-3: 84813741A/G
3-3: 84909687T/C
3-3: 84926100C/G
3-3: 85196011G/A
3-3: 85314733C/T
3-3: 85363986G/C
3-3: 85389096G/A
3-3: 85560113G/C
3-3: 85672705C/A
3-3: 85750028A/G
3-3: 85767724G/A
3-3: 85814395A/G
3-3: 85906571G/T
3-3: 85975005C/T
3-3: 86004676G/T
3-3: 86158909A/C
3-3: 86256405G/C
3-3: 86420865C/A
3-3: 86530215T/C
3-3: 86567586T/G
3-3: 86823655C/T
3-3: 86944283A/G
3-3: 87028702C/G
3-3: 87102218T/C
3-3: 87227295T/A
3-3: 87246923T/C
3-3: 87261871G/A
3-3: 87273756G/A
3-3: 87289626A/T
3-3: 87314795C/T
3-3: 87495010C/T
3-3: 87535847T/C
3-3: 87557227G/A
3-3: 87741793C/T
3-3: 87879331C/T
3-3: 87972546T/A
3-3: 88041796C/T
3-3: 88221727G/A
3-3: 88300646C/A
3-3: 88554963A/G
3-3: 88690575T/A
3-3: 88785919A/G
3-3: 88831625G/A
3-3: 88892693T/A
3-3: 89099575C/T
3-3: 89186820T/C
3-3: 89334726A/C
3-3: 89441321C/G
3-3: 89443421C/T
3-3: 89507896A/G
3-3: 90307053G/A
3-3: 90380814A/T
3-3: 90406079T/A
3-3: 90450998C/T
3-3: 90451950G/C
3-3: 90451999A/T
3-3: 90607374T/A
3-3: 90981085G/A
3-3: 91125896G/A
3-4: 28169C/T
3-4: 428540A/G
3-4: 552630T/C
3-4: 589848C/T
3-4: 734733T/C
3-4: 1021665C/A
3-4: 1207344G/A
3-4: 1232433C/T
3-4: 1232953A/G
3-4: 1251167T/C
3-4: 1300317T/C
3-4: 1326424G/A
3-4: 1344229G/T
3-4: 1359832A/G
3-4: 1385620T/G
3-4: 1397227C/G
3-4: 1397784T/C
3-4: 1450347C/G
3-4: 1454374T/C
3-4: 1468051T/G
3-4: 1478182A/G
3-4: 1492791C/A
3-4: 1662572G/A
3-4: 1667454T/C
3-4: 1677711T/C
3-4: 1680066T/C
3-4: 1693703A/T
3-4: 1704014G/A
3-4: 1877185C/T
3-4: 1897252G/A
3-4: 1968732A/C
3-4: 2219004G/A
3-4: 2403895T/C
3-4: 2460364G/C
3-4: 2578637C/T
3-4: 2625317C/G
3-4: 2625979T/G
3-4: 2676739T/C
3-4: 2772205A/G
3-4: 2853652T/C
3-4: 2886602G/T
3-4: 2971341T/G
3-4: 3167976A/G
3-4: 3265658A/G
3-4: 3346672A/T
3-4: 3558775T/C
3-4: 3564875T/G
3-4: 3593360T/A
3-4: 3677482T/C
3-4: 3761280A/G
3-4: 3789158A/T
3-4: 4031876C/A
3-4: 4054909C/T
3-4: 4136763C/T
3-4: 4273270C/T
3-4: 4295200A/G
3-4: 4306697C/A
3-4: 4363064G/C
3-4: 4363656C/A
3-4: 4363688T/C
3-4: 4390721G/A
3-4: 4430427C/T
3-4: 4436394T/C
3-4: 4461391T/G
3-4: 4484462G/A
3-4: 4500725T/G
3-4: 4504809T/C
3-4: 4514408A/C
3-4: 4525204G/A
3-4: 4536413A/T
3-4: 4555210G/A
3-4: 4652973T/A
3-4: 4691118C/A
3-4: 4704263C/T
3-4: 4764548A/G
3-4: 4797618C/A
3-4: 4814207C/T
3-4: 4936149T/G
3-4: 4950985C/G
3-4: 5048830A/G
3-4: 5167319A/G
3-4: 5290875T/A
3-4: 5311493A/C
3-4: 5398304T/C
3-4: 5415199A/C
3-4: 5498539A/G
3-4: 5652484A/C
3-4: 5714029T/C
3-4: 5734612C/T
3-4: 5764851G/A
3-4: 5767707G/T
3-4: 5907488A/C
3-4: 6012078T/C
3-4: 6083853C/T
3-4: 6311908A/G
3-4: 6428966T/A
3-4: 6564284T/A
3-4: 6716525G/T
3-4: 6730832A/G
3-4: 6772115C/T
3-4: 6809074C/T
3-4: 6852069C/T
3-4: 6914427C/T
3-4: 6986604T/C
3-4: 7084560T/A
3-4: 7102202C/G
3-4: 7141482A/G
3-4: 7186741T/A
3-4: 7215073T/A
3-4: 7392208T/A
3-4: 7392762G/A
3-4: 7396781T/G
3-4: 7396904T/C
3-4: 7407122G/A
3-4: 7471198T/G
3-4: 7512410A/T
3-4: 7567451G/A
3-4: 7569300C/T
3-4: 7595825T/A
3-4: 7671100C/T
3-4: 7700856G/A
3-4: 7736013A/C
3-4: 7833651T/C
3-4: 7913236A/T
3-4: 7939457C/T
3-4: 8045530C/G
3-4: 8052025T/C
3-4: 8141848A/C
3-4: 8190489C/G
3-4: 8269260T/A
3-4: 8322368C/A
3-4: 8322393A/C
3-4: 8322524A/G
3-4: 8382136C/T
3-4: 8468428A/T
3-4: 8505364T/A
3-4: 8547580C/T
3-4: 8549682T/A
3-4: 8560884T/C
3-4: 8573950A/G
3-4: 8592043A/T
3-4: 8689266A/T
3-4: 8690385C/T
3-4: 8714842T/G
3-4: 8806102C/G
3-4: 8842124A/G
3-4: 8872143T/C
3-4: 8900210C/A
3-4: 8911603A/G
3-4: 8979616T/C
3-4: 9018589C/T
3-4: 9034406T/A
3-4: 9034514C/A
3-4: 9035191T/C
3-4: 9035236A/G
3-4: 9035283A/T
3-4: 9035306T/G
3-4: 9035309G/T
3-4: 9035321C/A
3-4: 9037561T/C
3-4: 9205189G/A
3-4: 9231255A/G
3-4: 9281007C/A
3-4: 9315200T/A
3-4: 9320463G/A
3-4: 9345385A/T
3-4: 9358580T/G
3-4: 9490918T/G
3-4: 9519315A/G
3-4: 9612144C/T
3-4: 9655298A/T
3-4: 9684582A/G
3-4: 9735056T/G
3-4: 9746547T/A
3-4: 9778830A/G
3-4: 9872346G/A
3-4: 9872452G/A
3-4: 9872600A/G
3-4: 9901886G/C
3-4: 9913075T/A
3-4: 9938970A/T
3-4: 10098914C/T
3-4: 10115305A/T
3-4: 10140797T/C
3-4: 10207086A/T
3-4: 10209166G/A
3-4: 10220826C/T
3-4: 10234945A/G
3-4: 10277837T/C
3-4: 10296701T/C
3-4: 10296963G/A
3-4: 10364170T/C
3-4: 10403127T/C
3-4: 10472148C/T
3-4: 10516710C/A
3-4: 10526470G/A
3-4: 10526736A/C
3-4: 10526781T/C
3-4: 10526854T/C
3-4: 10526976T/C
3-4: 10527222T/G
3-4: 10527292A/G
3-4: 10553058A/C
3-4: 10705052T/C
3-4: 10725385C/T
3-4: 10743349T/C
3-4: 10781594T/A
3-4: 10850803C/A
3-4: 10877463G/A
3-4: 10896412A/C
3-4: 10979771C/T
3-4: 11041571C/A
3-4: 11059842G/A
3-4: 11190975C/T
3-4: 11216122A/G
3-4: 11261233T/C
3-4: 11417006G/A
3-4: 11436452T/C
3-4: 11497394T/A
3-4: 11503485T/A
3-4: 11513497C/T
3-4: 11634342C/A
3-4: 11642838T/A
3-4: 11683064A/G
3-4: 11708752G/A
3-4: 11766467A/G
3-4: 11768480A/T
3-4: 11826058A/G
3-4: 11919903G/A
3-4: 11933088G/A
3-4: 11944019T/A
3-4: 11991283C/T
3-4: 12023197C/A
3-4: 12061726T/G
3-4: 12124407T/A
3-4: 12124976T/C
3-4: 12136933G/A
3-4: 12174717G/A
3-4: 12174967C/T
3-4: 12203534T/G
3-4: 12248194T/A
3-4: 12249418G/A
3-4: 12273992G/A
3-4: 12341324G/A
3-4: 12389841A/G
3-4: 12402809C/G
3-4: 12404395T/G
3-4: 12405765T/A
3-4: 12406186C/T
3-4: 12406290A/C
3-4: 12406522T/C
3-4: 12430096T/A
3-4: 12468118T/G
3-4: 12515340G/A
3-4: 12525956C/A
3-4: 12557621T/A
3-4: 12627554A/G
3-4: 12652776T/C
3-4: 12671018G/A
3-4: 12728902C/T
3-4: 12765698A/T
3-4: 12813869C/T
3-4: 12853582G/A
3-4: 12913971G/A
3-4: 13048272C/T
3-4: 13086093T/A
3-4: 13309950T/C
3-4: 13366291G/A
3-4: 13384439T/C
3-4: 13411238T/C
3-4: 13421990G/A
3-4: 13466109A/G
3-4: 13517921T/A
3-4: 13568952C/T
3-4: 13633536A/G
3-4: 13723294T/A
3-4: 13773532C/G
3-4: 13869716A/G
3-4: 14045545G/A
3-4: 14048670G/A
3-4: 14153729A/T
3-4: 14166580A/G
3-4: 14166595C/T
3-4: 14178086T/C
3-4: 14184228A/T
3-4: 14188440T/A
3-4: 14236751G/A
3-4: 14239776C/A
3-4: 14247867A/G
3-4: 14255556G/T
3-4: 14267638T/C
3-4: 14278414T/C
3-4: 14295470G/A
3-4: 14306064A/C
3-4: 14371833G/A
3-4: 14385136G/T
3-4: 14502504G/A
3-4: 14518475C/T
3-4: 14563432T/G
3-4: 14583549G/A
3-4: 14603611C/A
3-4: 14669298C/T
3-4: 14679606T/A
3-4: 14743308T/C
3-4: 14758076G/A
3-4: 14853284G/A
3-4: 14861293A/G
3-4: 14861410A/T
3-4: 14865696G/A
3-4: 14866326T/C
3-4: 14867194A/G
3-4: 14876372T/A
3-4: 14881485A/G
3-4: 14891817C/T
3-4: 14902513G/A
3-4: 14902618T/C
3-4: 14904980C/T
3-4: 14905138C/T
3-4: 14905290A/G
3-4: 14913861T/A
3-4: 14941357T/C
3-4: 14955870G/A
3-4: 15026543G/T
3-4: 15103467C/T
3-4: 15108112G/A
3-4: 15124152G/A
3-4: 15125370C/G
3-4: 15131463A/C
3-4: 15182628T/A
3-4: 15200525C/T
3-4: 15210579A/T
3-4: 15238471T/A
3-4: 15257379A/G
3-4: 15271874G/A
3-4: 15281669A/T
3-4: 15281937G/A
3-4: 15346647A/G
3-4: 15394943G/T
3-4: 15462746T/C
3-4: 15503367T/A
3-4: 15602069G/A
3-4: 15634122G/A
3-4: 15659936C/T
3-4: 15718801T/C
3-4: 15746399C/G
3-4: 15784272C/T
3-4: 15784309T/C
3-4: 15784422C/T
3-4: 15784474G/A
3-4: 15784556G/T
3-4: 15796786T/C
3-4: 15812905C/T
3-4: 15825157C/T
3-4: 15825253T/C
3-4: 15825268G/A
3-4: 15828608A/.
3-4: 15829005C/T
3-4: 15945615G/A
3-4: 16068020G/A
3-4: 16099747A/C
3-4: 16157851C/T
3-4: 16224618T/C
3-4: 16298059G/A
3-4: 16317095A/C
3-4: 16422241A/T
3-4: 16498668T/A
3-4: 16500474A/G
3-4: 16507933G/A
3-4: 16511158A/C
3-4: 16521255A/T
3-4: 16521301T/C
3-4: 16525297G/A
3-4: 16557504A/T
3-4: 16625450G/A
3-4: 16631208C/T
3-4: 16631219A/C
3-4: 16631427A/T
3-4: 16641343A/G
3-4: 16656812C/T
3-4: 16737358A/G
3-4: 16768529T/A
3-4: 16806977G/C
3-4: 16855631G/T
3-4: 16896348T/C
3-4: 16981804G/C
3-4: 17027068C/T
3-4: 17102223G/A
3-4: 17117374T/A
3-4: 17117397C/T
3-4: 17121541G/A
3-4: 17122070C/G
3-4: 17149157G/A
3-4: 17224397C/A
3-4: 17251833T/C
3-4: 17266975G/T
3-4: 17289587G/A
3-4: 17497754C/A
3-4: 17521439C/A
3-4: 17531621T/C
3-4: 17584654G/A
3-4: 17586947A/G
3-4: 17587057A/C
3-4: 17602672G/A
3-4: 17618912G/C
3-4: 17672504G/A
3-4: 17720844G/T
3-4: 17788093A/G
3-4: 17833723T/G
3-4: 17838225C/G
3-4: 17856024G/A
3-4: 17857252A/G
3-4: 17924574G/A
3-4: 17973834C/A
3-4: 17976534A/T
3-4: 17976621G/A
3-4: 17998874T/G
3-4: 18011567G/A
3-4: 18147383T/A
3-4: 18147514C/T
3-4: 18148600C/T
3-4: 18149530G/A
3-4: 18192703C/T
3-4: 18222230G/T
3-4: 18333026G/A
3-4: 18334606G/T
3-4: 18363851T/C
3-4: 18410627T/G
3-4: 18442688T/A
3-4: 18557940A/T
3-4: 18560445G/T
3-4: 18572381C/A
3-4: 18580777C/T
3-4: 18582814A/G
3-4: 18606191G/T
3-4: 18626707T/C
3-4: 18681099G/T
3-4: 18691237T/C
3-4: 18707825G/T
3-4: 18765083A/G
3-4: 18824303G/T
3-4: 18939051G/A
3-4: 18978004T/C
3-4: 19108694T/G
3-4: 19211223G/A
3-4: 19378713C/T
3-4: 19384742T/C
3-4: 19468791G/C
3-4: 19475865A/G
3-4: 19581779A/G
3-4: 19584386G/C
3-4: 19622147T/C
3-4: 19625755C/T
3-4: 19644154C/T
3-4: 19673289C/T
3-4: 19698569G/A
3-4: 19774860T/C
3-4: 19829843C/T
3-4: 19877457T/C
3-4: 19932887T/C
3-4: 19969949T/A
3-4: 20075779C/T
3-4: 20101006G/C
3-4: 20104397G/T
3-4: 20136011G/A
3-4: 20210321G/A
3-4: 20245288T/C
3-4: 20247597A/T
3-4: 20248465G/A
3-4: 20248747A/G
3-4: 20249230G/T
3-4: 20249295C/A
3-4: 20249481A/C
3-4: 20308847C/T
3-4: 20390532T/C
3-4: 20421239C/G
3-4: 20447476A/T
3-4: 20501462C/T
3-4: 20600046T/A
3-4: 20746234T/A
3-4: 20793086T/A
3-4: 20852390A/T
3-4: 20959748G/A
3-4: 21031627G/A
3-4: 21077168C/T
3-4: 21091624G/C
3-4: 21128730T/A
3-4: 21160072G/T
3-4: 21252991G/A
3-4: 21254113G/A
3-4: 21287509A/T
3-4: 21366489A/T
3-4: 21622392A/T
3-4: 21789421T/A
3-4: 21792078A/G
3-4: 21809897G/T
3-4: 21837408C/G
3-4: 21866184T/G
3-4: 22002762T/C
3-4: 22025621G/A
3-4: 22180326G/C
3-4: 22224782T/A
3-4: 22266013C/T
3-4: 22316126A/T
3-4: 22411482G/T
3-4: 22460656A/G
3-4: 22533134C/A
3-4: 22621712C/G
3-4: 22712336T/A
3-4: 22827006A/T
3-4: 22851641C/G
3-4: 22859771C/G
3-4: 22897780A/G
3-4: 22908304G/A
3-4: 22918512A/G
3-4: 22955423G/C
3-4: 22972282C/T
3-4: 23003320T/C
3-4: 23009227A/C
3-4: 23010292T/C
3-4: 23018126T/C
3-4: 23040946C/A
3-4: 23072989A/G
3-4: 23115330T/C
3-4: 23124296G/A
3-4: 23167693A/T
3-4: 23210659A/G
3-4: 23253065A/G
3-4: 23282861C/A
3-4: 23312888C/T
3-4: 23327473A/T
3-4: 23359875G/T
3-4: 23458529T/C
3-4: 23514018C/T
3-4: 23558692C/T
3-4: 23560762T/C
3-4: 23563425G/A
3-4: 23563716C/T
3-4: 23564481A/G
3-4: 23574601G/A
3-4: 23600032A/C
3-4: 23676891A/T
3-4: 23718906T/C
3-4: 23749587G/C
3-4: 23781687T/G
3-4: 23781877G/A
3-4: 23781902G/T
3-4: 23782098T/G
3-4: 23795099A/G
3-4: 23835406G/A
3-4: 23846107T/A
3-4: 23867263C/T
3-4: 23946452C/T
3-4: 23946645C/T
3-4: 23957970A/C
3-4: 23970224A/G
3-4: 24071639C/T
3-4: 24110912T/A
3-4: 24125425T/C
3-4: 24239565A/G
3-4: 24257228G/A
3-4: 24366527C/T
3-4: 24471441C/T
3-4: 24484317C/A
3-4: 24501491G/A
3-4: 24514613A/C
3-4: 24568986C/T
3-4: 24618512T/A
3-4: 24712641T/A
3-4: 24853555C/G
3-4: 24863857T/C
3-4: 24865102C/A
3-4: 24865342C/T
3-4: 24885210C/G
3-4: 24895289A/T
3-4: 24934824T/C
3-4: 24957315T/G
3-4: 24993102G/A
3-4: 25053847G/A
3-4: 25090109G/A
3-4: 25122529T/A
3-4: 25169215A/G
3-4: 25226192T/C
3-4: 25244403A/G
3-4: 25332634A/G
3-4: 25396086C/T
3-4: 25396089T/G
3-4: 25396099T/A
3-4: 25396111T/C
3-4: 25396113G/A
3-4: 25396115G/A
3-4: 25396120G/T
3-4: 25396152C/G
3-4: 25396170A/T
3-4: 25396193C/T
3-4: 25433521G/T
3-4: 25437364A/T
3-4: 25443838A/G
3-4: 25514590T/A
3-4: 25619973T/C
3-4: 25641613G/A
3-4: 25649773T/C
3-4: 25780903T/C
3-4: 25783472G/A
3-4: 25878816G/T
3-4: 25921130A/C
3-4: 25933863C/T
3-4: 25972640A/G
3-4: 25974987G/A
3-4: 26094937G/A
3-4: 26096748A/G
3-4: 26245639T/A
3-4: 26304185G/T
3-4: 26360837T/G
3-4: 26396179G/A
3-4: 26463018G/A
3-4: 26495440T/C
3-4: 26559726G/A
3-4: 26562117A/T
3-4: 26576803A/T
3-4: 26603277G/T
3-4: 26603383T/C
3-4: 26603572T/C
3-4: 26639874A/G
3-4: 26682698T/C
3-4: 26714252A/G
3-4: 26756076T/C
3-4: 26782239G/T
3-4: 26868817T/C
3-4: 26908647G/A
3-4: 27010062G/A
3-4: 27072327A/G
3-4: 27087075C/T
3-4: 27129030G/A
3-4: 27181921A/G
3-4: 27265422C/G
3-4: 27268246T/C
3-4: 27269138T/C
3-4: 27287342G/A
3-4: 27369250T/G
3-4: 27384384G/A
3-4: 27395628G/T
3-4: 27413348T/C
3-4: 27417301C/T
3-4: 27423535A/T
3-4: 27485199C/T
3-4: 27526090A/T
3-4: 27585936C/T
3-4: 27667858T/C
3-4: 27717116T/C
3-4: 27729549G/C
3-4: 27786873C/T
3-4: 27858848C/A
3-4: 27934672T/C
3-4: 27950329A/G
3-4: 27985160T/C
3-4: 28026569A/T
3-4: 28066634C/T
3-4: 28066735A/G
3-4: 28068302A/T
3-4: 28096197T/A
3-4: 28139182A/T
3-4: 28220071G/A
3-4: 28274208A/G
3-4: 28444010T/C
3-4: 28454668C/T
3-4: 28503550A/T
3-4: 28555663T/C
3-4: 28586787G/A
3-4: 28621441T/C
3-4: 28823419T/C
3-4: 28991786T/C
3-4: 28996552A/G
3-4: 29058828A/C
3-4: 29071768G/A
3-4: 29086794T/A
3-4: 29146131C/T
3-4: 29170280A/G
3-4: 29225872C/T
3-4: 29281734G/A
3-4: 29306931G/A
3-4: 29319718C/T
3-4: 29361051C/T
3-4: 29371229A/C
3-4: 29371491C/A
3-4: 29409559A/T
3-4: 29455537T/C
3-4: 29476097C/T
3-4: 29484460G/A
3-4: 29487315C/A
3-4: 29514421T/C
3-4: 29526056T/C
3-4: 29552692A/G
3-4: 29565119G/A
3-4: 29609607A/G
3-4: 29623019T/A
3-4: 29684977C/T
3-4: 29780886G/T
3-4: 29845733T/C
3-4: 29857491G/A
3-4: 29880679C/T
3-4: 29937230T/C
3-4: 29986147G/T
3-4: 30065218C/T
3-4: 30206891C/T
3-4: 30209197G/A
3-4: 30246278G/A
3-4: 30381974G/A
3-4: 30403131G/T
3-4: 30568135T/G
3-4: 30643719T/C
3-4: 30655554A/C
3-4: 30665521A/G
3-4: 30687489G/A
3-4: 30868522G/A
3-4: 30906834A/G
3-4: 30978904T/G
3-4: 31101518C/T
3-4: 31137046C/A
3-4: 31389167G/T
3-4: 31541883G/A
3-4: 31556403A/G
3-4: 31615169A/T
3-4: 31804014A/G
3-4: 31846557C/T
3-4: 31906006C/G
3-4: 31979870G/A
3-4: 31999146C/T
3-4: 32049192A/G
3-4: 32157004C/T
3-4: 32240751A/T
3-4: 32263186T/C
3-4: 32277573A/G
3-4: 32370872G/C
3-4: 32402542G/A
3-4: 32486602G/T
3-4: 32518852T/A
3-4: 32610357T/G
3-4: 32865820A/G
3-4: 32987319G/A
3-4: 33062569C/T
3-4: 33142702A/G
3-4: 33192003G/A
3-4: 33192527T/A
3-4: 33192549G/A
3-4: 33193993C/T
3-4: 33246791C/T
3-4: 33388169T/C
3-4: 33611044T/C
3-4: 33676967C/A
3-4: 33736616G/A
3-4: 33783798T/G
3-4: 33812351T/A
3-4: 33812511G/A
3-4: 33812664A/G
3-4: 33812976A/T
3-4: 33840399C/G
3-4: 33898877C/T
3-4: 33951709A/T
3-4: 34032000C/T
3-4: 34086666T/G
3-4: 34170097T/C
3-4: 34220462A/C
3-4: 34309582C/G
3-4: 34348251A/G
3-4: 34396750C/T
3-4: 34411308A/T
3-4: 34451925C/A
3-4: 34494587C/A
3-4: 34528698A/G
3-4: 34560736G/A
3-4: 34595126A/G
3-4: 34631167G/A
3-4: 34644285T/C
3-4: 34661041C/T
3-4: 34676328G/A
3-4: 34730588A/G
3-4: 34777727C/T
3-4: 34802682T/A
3-4: 34818603C/T
3-4: 34829900G/T
3-4: 34851477A/T
3-4: 34863781C/T
3-4: 34925535C/T
3-4: 35019346A/G
3-4: 35030116A/G
3-4: 35157794C/T
3-4: 35235274C/A
3-4: 35330388C/T
3-4: 35391830G/A
3-4: 35479859C/T
3-4: 35533492T/C
3-4: 35558761C/T
3-4: 35651344G/A
3-4: 35665456A/T
3-4: 35699742A/T
3-4: 35701953C/T
3-4: 35794914G/A
3-4: 35838630A/C
3-4: 35884187T/A
3-4: 35982007G/T
3-4: 35991036G/A
3-4: 35991238G/A
3-4: 35999869A/T
3-4: 36000008C/T
3-4: 36000118G/A
3-4: 36000368T/A
3-4: 36000378C/T
3-4: 36000575T/A
3-4: 36005490C/G
3-4: 36011702A/G
3-4: 36047805G/A
3-4: 36101299T/G
3-4: 36115455G/C
3-4: 36145805T/A
3-4: 36166319A/T
3-4: 36183657T/C
3-4: 36246115C/A
3-4: 36276021T/A
3-4: 36314750T/G
3-4: 36329162G/A
3-4: 36372073T/C
3-4: 36565224C/T
3-4: 36598180A/G
3-4: 36624583C/T
3-4: 36657087A/G
3-4: 36659092G/A
3-4: 36661834T/A
3-4: 36662065T/G
3-4: 36662127G/A
3-4: 36662147A/G
3-4: 36662597A/G
3-4: 36662666A/T
3-4: 36663185T/C
3-4: 36753251C/A
3-4: 36806711A/T
3-4: 36907373G/T
3-4: 36937669G/T
3-4: 36948383G/A
3-4: 37004438C/T
3-4: 37044466G/A
3-4: 37081096C/T
3-4: 37119473T/A
3-4: 37147976G/A
3-4: 37194788G/C
3-4: 37433601A/G
3-4: 37502137G/A
3-4: 37514385C/A
3-4: 37535161C/T
3-4: 37549813G/A
3-4: 37577244T/C
3-4: 37665756A/T
3-4: 37756974C/T
3-4: 37800060C/G
3-4: 37813184G/A
3-4: 37841226T/C
3-4: 37884484T/A
3-4: 37903710G/T
3-4: 37922826T/G
3-4: 38010872G/A
3-4: 38122087G/A
3-4: 38132494C/T
3-4: 38132953C/.
3-4: 38204912A/G
3-4: 38402862G/A
3-4: 38499062T/A
3-4: 38510827A/T
3-4: 38524456G/A
3-4: 38580000C/T
3-4: 38598944A/T
3-4: 38664762G/A
3-4: 38852735A/C
3-4: 39021468C/T
3-4: 39040084C/T
3-4: 39052100T/A
3-4: 39205687G/T
3-4: 39341821A/G
3-4: 39346773A/G
3-4: 39346860G/C
3-4: 39375145T/A
3-4: 39376802T/G
3-4: 39376926C/T
3-4: 39377560G/A
3-4: 39391084C/A
3-4: 39470034C/T
3-4: 39508013C/T
3-4: 39509315T/C
3-4: 39519328C/T
3-4: 39532136C/T
3-4: 39541534T/C
3-4: 39547084G/T
3-4: 39579853A/T
3-4: 39658325T/C
3-4: 39682456T/C
3-4: 39697740C/A
3-4: 39728401G/A
3-4: 39827010T/C
3-4: 39864050C/A
3-4: 39963900C/T
3-4: 40019252A/C
3-4: 40127293T/C
3-4: 40139623G/T
3-4: 40219499T/A
3-4: 40246135T/C
3-4: 40275293A/C
3-4: 40328662A/G
3-4: 40394742A/G
3-4: 40406397G/A
3-4: 40437994C/T
3-4: 40608277G/A
3-4: 40637772T/C
3-4: 40720862G/A
3-4: 40762831G/A
3-4: 40819326C/T
3-4: 40948919T/C
3-4: 41017488G/T
3-4: 41126457G/A
3-4: 41183086A/G
3-4: 41233489T/C
3-4: 41291711T/C
3-4: 41438197T/G
3-4: 41469887T/C
3-4: 41513684C/A
3-4: 41556435T/C
3-4: 41584000C/A
3-4: 41605301A/T
3-4: 41610549T/G
3-4: 41610650C/T
3-4: 41615040G/C
3-4: 41616160T/C
3-4: 41616208C/T
3-4: 41616364T/A
3-4: 41619423C/T
3-4: 41707558C/G
3-4: 41713718T/A
3-4: 41725603T/A
3-4: 41760303C/T
3-4: 42156551G/A
3-4: 42360129T/G
3-4: 42361559A/G
3-4: 42370280G/C
3-4: 42382536C/T
3-4: 42396563C/A
3-4: 42419177C/T
3-4: 42497213A/G
3-4: 42516968G/T
3-4: 42533147G/T
3-4: 42551562A/G
3-4: 42565966G/A
3-4: 42576005T/G
3-4: 42603008T/C
3-4: 42609515T/C
3-4: 42618091C/T
3-4: 42648213T/G
3-4: 42674174A/G
3-4: 42687530T/A
3-4: 42716728G/A
3-4: 42805094G/A
3-4: 42881448G/C
3-4: 42907490A/T
3-4: 43038343G/A
3-4: 43038365G/A
3-4: 43038568C/.
3-4: 43073704G/T
3-4: 43095228A/G
3-4: 43097100C/T
3-4: 43097253T/C
3-4: 43097319A/T
3-4: 43098248A/T
3-4: 43098560T/C
3-4: 43107803A/G
3-4: 43112814G/A
3-4: 43115529A/T
3-4: 43116956A/G
3-4: 43119258A/G
3-4: 43130881G/A
3-4: 43143138T/C
3-4: 43170673T/C
3-4: 43247644T/C
3-4: 43248774C/T
3-4: 43261375T/A
3-4: 43291208A/G
3-4: 43293443T/C
3-4: 43293672C/A
3-4: 43300088C/T
3-4: 43300203C/T
3-4: 43300311T/A
3-4: 43305266C/T
3-4: 43306938A/C
3-4: 43310631T/G
3-4: 43311017C/T
3-4: 43312481T/C
3-4: 43325590G/C
3-4: 43338127C/G
3-4: 43357412G/A
3-4: 43357485T/A
3-4: 43370469T/G
3-4: 43370511G/A
3-4: 43371917T/A
3-4: 43450007A/C
3-4: 43521303T/G
3-4: 43537155A/G
3-4: 43578063T/C
3-4: 43626631C/T
3-4: 43643755G/A
3-4: 43658678A/T
3-4: 43726799T/G
3-4: 43786020T/G
3-4: 43846590G/A
3-4: 43867611G/A
3-4: 44008505A/G
3-4: 44010337C/T
3-4: 44021381C/T
3-4: 44048985G/A
3-4: 44061133G/A
3-4: 44072259A/G
3-4: 44084182G/A
3-4: 44085778C/A
3-4: 44098684G/C
3-4: 44237490G/T
3-4: 44252183C/T
3-4: 44270301T/C
3-4: 44282981G/A
3-4: 44312528G/A
3-4: 44334378T/C
3-4: 44334808T/G
3-4: 44352235G/T
3-4: 44367895A/C
3-4: 44414633G/A
3-4: 44431258C/T
3-4: 44493712G/A
3-4: 44590844A/C
3-4: 44642345A/T
3-4: 44643647T/C
3-4: 44782136C/T
3-4: 44817669G/T
3-4: 44819269T/C
3-4: 44819339A/C
3-4: 44819375C/T
3-4: 44828942T/C
3-4: 44829368C/T
3-4: 44829914C/A
3-4: 44830176C/T
3-4: 44830201C/G
3-4: 44830602C/T
3-4: 44830848C/T
3-4: 44831680T/G
3-4: 44833471T/C
3-4: 44833663T/C
3-4: 44833850G/T
3-4: 44837224C/A
3-4: 44839318T/C
3-4: 44872761C/G
3-4: 44899254A/T
3-4: 44950859C/T
3-4: 44966948T/C
3-4: 44973773C/T
3-4: 44992246G/C
3-4: 45012776A/G
3-4: 45120321G/A
3-4: 45146870A/C
3-4: 45208806G/T
3-4: 45255756T/C
3-4: 45332243T/C
3-4: 45405515A/G
3-4: 45467167C/A
3-4: 45476228C/T
3-4: 45478510A/G
3-4: 45536065T/C
3-4: 45563570G/A
3-4: 45572990C/T
3-4: 45584678A/G
3-4: 45603275T/G
3-4: 45603469T/C
3-4: 45604997C/G
3-4: 45605859T/C
3-4: 45606047G/A
3-4: 45620962A/T
3-4: 45646834G/C
3-4: 45651095T/C
3-4: 45652099A/C
3-4: 45661639T/C
3-4: 45678189T/C
3-4: 45692135T/C
3-4: 45704936T/C
3-4: 45782801G/A
3-4: 45836982T/G
3-4: 45909707G/A
3-4: 45927794G/T
3-4: 45937212C/T
3-4: 45942188C/T
3-4: 45974242C/T
3-4: 46013600A/G
3-4: 46044329T/C
3-4: 46084987C/T
3-4: 46091305G/A
3-4: 46094770C/A
3-4: 46097206T/A
3-4: 46112291T/G
3-4: 46222999C/T
3-4: 46224609A/T
3-4: 46225537A/C
3-4: 46225545A/T
3-4: 46226535T/C
3-4: 46238092A/C
3-4: 46263182A/G
3-4: 46267847A/G
3-4: 46301383G/A
3-4: 46399516T/C
3-4: 46498280T/C
3-4: 46596383C/A
3-4: 46684298T/G
3-4: 46780968A/G
3-4: 46806612C/T
3-4: 46842658A/T
3-4: 46892761A/C
3-4: 46949283T/C
3-4: 46992295A/G
3-4: 47002071A/C
3-4: 47016053A/C
3-4: 47067942T/A
3-4: 47127800A/G
3-4: 47257394T/C
3-4: 47262215C/A
3-4: 47284445G/A
3-4: 47345250T/C
3-4: 47371913G/A
3-4: 47414946A/G
3-4: 47504336A/T
3-4: 47515608G/T
3-4: 47541188G/A
3-4: 47570139G/T
3-4: 47582031G/T
3-4: 47693986A/T
3-4: 47737726T/C
3-4: 47738996C/T
3-4: 47767845T/C
3-4: 47819641G/A
3-4: 47842606T/A
3-4: 47845378A/C
3-4: 47862757T/A
3-4: 47902225G/A
3-4: 47928673A/T
3-4: 47971813A/C
3-4: 47979332C/A
3-4: 48025451C/G
3-4: 48112308C/T
3-4: 48128919A/G
3-4: 48231376C/T
3-4: 48295028C/T
3-4: 48371645T/C
3-4: 48382633G/A
3-4: 48384113T/C
3-4: 48421797C/T
3-4: 48468729G/A
3-4: 48468804A/T
3-4: 48468806T/.
3-4: 48468819T/.
3-4: 48468831A/.
3-4: 48468916G/A
3-4: 48469082C/T
3-4: 48469524C/T
3-4: 48469564A/G
3-4: 48469613T/A
3-4: 48469644C/T
3-4: 48510467G/A
3-4: 48511149T/C
3-4: 48516629T/A
3-4: 48525341T/G
3-4: 48536423A/T
3-4: 48554724A/T
3-4: 48576463T/A
3-4: 48622161T/C
3-4: 48645787A/C
3-4: 48665781A/T
3-4: 48669892A/G
3-4: 48669916A/G
3-4: 48757088C/G
3-4: 48788709T/C
3-4: 48836165G/T
3-4: 48875715T/A
3-4: 48907565G/A
3-4: 48927221G/C
3-4: 48937262G/C
3-4: 48954679A/G
3-4: 48984762A/G
3-4: 48998750A/T
3-4: 49034455A/T
3-4: 49034589G/T
3-4: 49036088T/C
3-4: 49043035T/A
3-4: 49043287G/C
3-4: 49061399T/C
3-4: 49095827A/G
3-4: 49097502G/A
3-4: 49106574C/T
3-4: 49106676C/T
3-4: 49123931G/C
3-4: 49140316G/A
3-4: 49257503C/T
3-4: 49284785T/C
3-4: 49304793T/A
3-4: 49323103T/C
3-4: 49357181C/T
3-4: 49412915G/T
3-4: 49462482T/C
3-4: 49508327C/T
3-4: 49569222G/A
3-4: 49583029C/T
3-4: 49584646C/G
3-4: 49584753G/A
3-4: 49584757G/C
3-4: 49584905T/A
3-4: 49589783C/G
3-4: 49600974C/T
3-4: 49609836G/T
3-4: 49609877T/C
3-4: 49611317A/T
3-4: 49611719G/A
3-4: 49611828T/G
3-4: 49621523T/C
3-4: 49637418C/T
3-4: 49648200G/A
3-4: 49658262A/G
3-4: 49672520G/T
3-4: 49704876T/C
3-4: 49727390T/C
3-4: 49802203A/G
3-4: 49806576A/G
3-4: 49840443C/A
3-4: 49869711G/A
3-4: 49882738C/T
3-4: 49890024C/T
3-4: 49892791C/A
3-4: 49893067T/C
3-4: 49893188G/A
3-4: 49894493G/A
3-4: 49896190A/T
3-4: 49904188C/T
3-4: 49905577G/T
3-4: 49905626C/A
3-4: 49917605G/C
3-4: 49951466G/A
3-4: 49974242T/G
3-4: 49986380T/C
3-4: 50027082T/C
3-4: 50037545A/G
3-4: 50094810C/T
3-4: 50125903G/A
3-4: 50171938A/T
3-4: 50225709G/A
3-4: 50274782C/A
3-4: 50293818G/A
3-4: 50304093C/T
3-4: 50312221T/A
3-4: 50315627A/G
3-4: 50327241G/T
3-4: 50327738C/T
3-4: 50350936A/G
3-4: 50362154C/T
3-4: 50447489C/A
3-4: 50489682G/T
3-4: 50489687A/G
3-4: 50493878A/G
3-4: 50530783C/A
3-4: 50580297T/C
3-4: 50582753A/T
3-4: 50598636T/G
3-4: 50609683T/C
3-4: 50886784G/A
3-4: 50902963G/T
3-4: 50920119G/A
3-4: 50970306A/C
3-4: 51011427A/G
3-4: 51040345C/T
3-4: 51067895A/G
3-4: 51071625C/T
3-4: 51088197A/G
3-4: 51127207G/A
3-4: 51149268A/G
3-4: 51151995A/G
3-4: 51152104T/G
3-4: 51204260T/A
3-4: 51244241C/A
3-4: 51265122C/T
3-4: 51265443A/G
3-4: 51302331A/G
3-4: 51342208A/T
3-4: 51360647T/C
3-4: 51365475G/A
3-4: 51375033T/A
3-4: 51376484A/G
3-4: 51385389A/G
3-4: 51385739T/A
3-4: 51386151T/A
3-4: 51396786T/A
3-4: 51402100G/A
3-4: 51402313C/A
3-4: 51435588A/T
3-4: 51508162T/C
3-4: 51574504T/C
3-4: 51586191G/C
3-4: 51587192T/C
3-4: 51597242G/A
3-4: 51650965G/C
3-4: 51744978C/T
3-4: 51756175T/C
3-4: 51770175G/A
3-4: 51792573G/A
3-4: 51812261C/T
3-4: 51834375A/G
3-4: 51839749A/C
3-4: 51847097A/T
3-4: 51877828A/G
3-4: 51905324T/C
3-4: 51917477A/C
3-4: 51927913A/G
3-4: 51952350A/G
3-4: 51993548T/G
3-4: 52010190T/C
3-4: 52070935G/A
3-4: 52186300C/T
3-4: 52209556C/T
3-4: 52232702T/A
3-4: 52294187C/T
3-4: 52357150A/T
3-4: 52392793A/G
3-4: 52408152A/G
3-4: 52419104A/G
3-4: 52437180C/T
3-4: 52515851T/C
3-4: 52565915A/G
3-4: 52591016A/T
3-4: 52601155A/T
3-4: 52628641C/A
3-4: 52633768G/.
3-4: 52640139G/C
3-4: 52640159G/A
3-4: 52640362T/C
3-4: 52644920C/G
3-4: 52656838G/T
3-4: 52715020C/T
3-4: 52760762T/C
3-4: 52878265G/C
3-4: 52911414G/A
3-4: 52956370G/A
3-4: 52997345T/C
3-4: 53051562G/T
3-4: 53051907G/A
3-4: 53073882T/G
3-4: 53126929T/C
3-4: 53165550T/C
3-4: 53194436G/T
3-4: 53279928G/A
3-4: 53350608C/T
3-4: 53383107A/G
3-4: 53477775G/A
3-4: 53537873A/T
3-4: 53565616G/A
3-4: 53640512T/A
3-4: 53806740A/G
3-4: 53831527A/G
3-4: 53832297T/C
3-4: 53865403A/G
3-4: 53901997A/C
3-4: 53946557A/G
3-4: 53958557A/T
3-4: 54071261G/A
3-4: 54078580G/A
3-4: 54129742T/C
3-4: 54186323A/G
3-4: 54201472G/T
3-4: 54217483C/T
3-4: 54218198G/A
3-4: 54314961C/T
3-4: 54345810G/T
3-4: 54382735T/C
3-4: 54476176C/T
3-4: 54600555T/G
3-4: 54634038C/T
3-4: 54644594T/C
3-4: 54661226A/C
3-4: 54671996T/C
3-4: 54722455T/C
3-4: 54825207A/T
3-4: 54929339T/A
3-4: 55002732G/C
3-4: 55118648C/T
3-4: 55128729A/C
3-4: 55138785C/T
3-4: 55139621A/C
3-4: 55139819A/G
3-4: 55140559T/A
3-4: 55140694G/A
3-4: 55142528C/T
3-4: 55143084G/C
3-4: 55158678G/A
3-4: 55181351G/A
3-4: 55253910A/T
3-4: 55254426A/G
3-4: 55255130A/T
3-4: 55255151G/A
3-4: 55271439T/A
3-4: 55282200G/T
3-4: 55416949T/C
3-4: 55512635C/T
3-4: 55523729A/G
3-4: 55546399A/G
3-4: 55649023G/A
3-4: 55720773T/A
3-4: 55747459T/C
3-4: 55790668T/C
3-4: 55826279G/C
3-4: 55846341T/C
3-4: 55899976C/T
3-4: 55911728T/A
3-4: 55911758A/G
3-4: 55929129G/A
3-4: 55994492A/T
3-4: 56030441A/G
3-4: 56095118A/T
3-4: 56096323C/T
3-4: 56143720T/A
3-4: 56175040C/T
3-4: 56206121G/T
3-4: 56267150T/C
3-4: 56351991C/T
3-4: 56381662T/C
3-4: 56525224G/T
3-4: 56573762T/G
3-4: 56603764G/A
3-4: 56658890A/G
3-4: 56684554A/T
3-4: 56786315G/C
3-4: 56839633C/T
3-4: 56851639T/C
3-4: 56909447T/G
3-4: 57037989G/A
3-4: 57128733G/C
3-4: 57140246A/G
3-4: 57159069T/G
3-4: 57188304T/C
3-4: 57282706T/A
3-4: 57321151A/G
3-4: 57349441G/C
3-4: 57453894C/T
3-4: 57539954C/T
3-4: 57610740G/A
3-4: 57665285T/C
3-4: 57666667G/A
3-4: 57703679T/A
3-4: 57821147C/T
3-4: 57832595G/A
3-4: 57911350T/A
3-4: 57962982A/G
3-4: 58036201T/C
3-4: 58088306A/G
3-4: 58137071T/C
3-4: 58137860C/T
3-4: 58177279T/A
3-4: 58213272G/C
3-4: 58287925G/T
3-4: 58357058G/T
3-4: 58390582G/A
3-4: 58487032C/T
3-4: 58557113T/C
3-4: 58678541G/A
3-4: 58678931T/C
3-4: 58679046T/A
3-4: 58679257C/G
3-4: 58681230A/G
3-4: 58697544C/T
3-4: 58723150A/G
3-4: 58763274A/T
3-4: 58783082C/T
3-4: 58803869A/T
3-4: 58878038C/T
3-4: 58929827C/T
3-4: 59042101A/G
3-4: 59084263A/G
3-4: 59121051T/C
3-4: 59170570T/C
3-4: 59232871A/T
3-4: 59262196G/T
3-4: 59343383T/C
3-4: 59513751A/T
3-4: 59622105A/G
3-4: 59680428G/T
3-4: 59698083A/T
3-4: 59738939G/C
3-4: 59858895T/G
3-4: 59868908G/A
3-4: 59929031A/T
3-4: 59941122T/C
3-4: 59952336C/T
3-4: 60002715C/A
3-4: 60021854G/A
3-4: 60034958G/A
3-4: 60096487A/C
3-4: 60126216T/G
3-4: 60130727A/G
3-4: 60157606C/T
3-4: 60199837T/C
3-4: 60225985C/T
3-4: 60244681G/T
3-4: 60255056A/G
3-4: 60345494G/C
3-4: 60356120T/C
3-4: 60379084A/C
3-4: 60480594G/C
3-4: 60487182G/A
3-4: 60506536T/C
3-4: 60671927T/A
3-4: 60794417T/A
3-4: 60870739G/A
3-4: 60927610A/G
3-4: 60977627A/G
3-4: 61080327C/T
3-4: 61107819C/T
3-4: 61139033T/A
3-4: 61224963G/T
3-4: 61261824C/T
3-4: 61313425A/T
3-4: 61340993G/C
3-4: 61380931C/A
3-4: 61401354G/T
3-4: 61564138T/A
3-4: 61565971A/G
3-4: 61651421T/C
3-4: 61682719T/C
3-4: 61757893T/A
3-4: 61849664G/A
3-4: 61853306A/G
3-4: 61918539A/G
3-4: 61969423T/A
3-4: 62091138G/T
3-4: 62150001T/C
3-4: 62188850G/T
3-4: 62260962A/G
3-4: 62313221A/T
3-4: 62388180A/G
3-4: 62458748G/A
3-4: 62567939T/C
3-4: 62591847A/G
3-4: 62604685A/G
3-4: 62611233T/C
3-4: 62622729C/T
3-4: 62677082C/A
3-4: 62710276T/A
3-4: 62742492T/A
3-4: 62767431G/A
3-4: 62815185A/C
3-4: 62897362C/T
3-4: 62961042C/T
3-4: 62971891T/C
3-4: 62985435T/C
3-4: 62995709G/A
3-4: 63015963T/A
3-4: 63045151A/T
3-4: 63074121G/A
3-4: 63129573A/G
3-4: 63151258C/T
3-4: 63151486A/G
3-4: 63166056T/C
3-4: 63177330G/A
3-4: 63181583C/T
3-4: 63190379T/C
3-4: 63202384C/T
3-4: 63221333A/T
3-4: 63275208A/G
3-4: 63319134G/A
3-4: 63339730G/A
3-4: 63423587A/C
3-4: 63449732A/G
3-4: 63484922C/T
3-4: 63562722G/T
3-4: 63603201C/T
3-4: 63840388G/A
3-4: 63874746T/C
3-4: 63904043A/G
3-4: 64022025G/A
3-4: 64060999C/A
3-4: 64089314G/A
3-4: 64214000T/C
3-4: 64268267C/G
3-4: 64446522G/C
3-4: 64481511C/T
3-4: 64525097T/C
3-4: 64609531C/T
3-4: 64737208T/G
3-4: 64737397A/C
3-4: 64737592A/T
3-4: 64739174T/C
3-4: 64739413T/C
3-4: 64776109C/T
3-4: 64787210T/G
3-4: 64918816C/T
3-4: 64941871G/A
3-4: 64984400T/A
3-4: 65069369T/C
3-4: 65143553C/T
3-4: 65333762T/C
3-4: 65358591C/T
3-4: 65389133A/T
3-4: 65484105T/C
3-4: 65509904G/A
3-4: 65607370G/C
3-4: 65637103G/A
3-4: 65668887C/T
3-4: 65690710C/T
3-4: 65775392C/A
3-4: 65913441C/T
3-4: 65975803G/A
3-4: 66019136G/A
3-4: 66054561A/G
3-4: 66184563T/A
3-4: 66310279A/G
3-4: 66377006A/T
3-4: 66428637T/C
3-4: 66502489C/T
3-4: 66528237G/A
3-4: 66626172G/A
3-4: 66652595T/C
3-4: 66700888A/G
3-4: 66747639A/T
3-4: 66929462C/T
3-4: 67092103T/C
3-4: 67092655C/T
3-4: 67093175C/G
3-4: 67094222A/G
3-4: 67097024T/C
3-4: 67097163T/A
3-4: 67097261T/C
3-4: 67097268C/A
3-4: 67098629T/A
3-4: 67099254T/G
3-4: 67099525G/T
3-4: 67101432C/A
3-4: 67114174G/A
3-4: 67325417T/A
3-4: 67343885T/C
3-4: 67371500A/G
3-4: 67402677C/T
3-4: 67435374T/G
3-4: 67510749A/G
3-4: 67619540G/A
3-4: 67636529T/A
3-4: 67674425T/C
3-4: 67701997A/G
3-4: 67745140A/G
3-4: 67789027A/C
3-4: 67916648C/T
3-4: 67969006C/G
3-4: 68057120T/C
3-4: 68118883A/C
3-4: 68279625C/T
3-4: 68292350C/T
3-4: 68307423T/A
3-4: 68349863C/T
3-4: 68409949C/T
3-4: 68499407T/A
3-4: 68508461C/G
3-4: 68573735C/G
3-4: 68622529T/C
3-4: 68668132T/C
3-4: 68761549A/C
3-4: 68806902A/T
3-4: 68829606A/G
3-4: 68832003T/C
3-4: 68860644T/A
3-4: 68875687C/T
3-4: 68916649T/A
3-4: 68941349A/G
3-4: 69060340A/G
3-4: 69082690T/A
3-4: 69144110C/T
3-4: 69219002G/A
3-4: 69389860G/C
3-4: 69495005A/G
3-4: 69499127G/A
3-4: 69531049T/C
3-4: 69597892G/A
3-4: 69719405C/T
3-4: 69757117G/A
3-4: 69760737A/G
3-4: 69784360C/T
3-4: 69785292C/A
3-4: 69794689C/T
3-4: 70091247T/C
3-4: 70251296T/C
3-4: 70341622A/C
3-4: 70409628A/G
3-4: 70461489C/A
3-4: 70528253C/T
3-4: 70699065C/T
3-4: 70728899T/C
3-4: 70770198G/A
3-4: 70784004A/G
3-4: 70795554T/G
3-4: 70931115T/C
3-4: 70958730C/G
3-4: 71016655G/A
3-4: 71062116C/T
3-4: 71105363A/G
3-4: 71151880G/A
3-4: 71301468A/G
3-4: 71391170C/T
3-4: 71468933C/A
3-4: 71725602T/C
3-4: 71923485C/A
3-4: 71989959C/T
3-4: 72178212C/T
3-4: 72252423A/G
3-4: 72329756G/A
3-4: 72579782C/T
3-4: 72696918A/G
3-4: 72713655T/A
3-4: 72724517T/A
3-4: 72743935C/T
3-4: 72951651G/A
3-4: 72978168G/A
3-4: 73025787A/T
3-4: 73051871G/A
3-4: 73094538C/G
3-4: 73131580G/A
3-4: 73303959T/A
3-4: 73310150A/G
3-4: 73432899A/G
3-4: 73657293C/T
3-4: 73855644A/T
3-4: 73869758T/C
3-4: 73908071T/C
3-4: 73928181G/T
3-4: 73954721A/G
3-4: 74105525C/T
3-4: 74109742C/T
3-4: 74134616G/T
3-4: 74269615G/A
3-4: 74393075A/G
3-4: 74522826T/G
3-4: 74527027G/A
3-4: 74631389G/A
3-4: 74692706G/A
3-4: 74706368C/T
3-4: 74752845T/C
3-4: 74790596A/T
3-4: 74859391G/A
3-4: 74925143G/A
3-4: 75115478C/A
3-4: 75348754T/C
3-4: 75550399T/C
3-4: 75649075G/A
3-4: 75770171A/G
3-4: 75957795A/T
3-4: 75975135A/G
3-4: 75986266A/T
3-4: 76025951A/G
3-4: 76075509A/G
3-4: 76157680C/T
3-4: 76242107T/C
3-4: 76260280A/G
3-4: 76338625T/G
3-4: 76371546A/G
3-4: 76497851T/C
3-4: 76648354T/G
3-4: 76708917T/C
3-4: 76754126A/T
3-4: 76869431C/G
3-4: 76875717T/A
3-4: 76923543G/A
3-4: 77109103T/C
3-4: 77140558C/A
3-4: 77177425G/T
3-4: 77188992G/T
3-4: 77204675T/A
3-4: 77254377T/C
3-4: 77254560C/T
3-4: 77277767G/A
3-4: 77307739C/A
3-4: 77483258G/A
3-4: 77501929G/T
3-4: 77565211A/G
3-4: 77800236A/T
3-4: 78254223A/T
3-4: 78300546G/A
3-4: 78422155A/T
3-4: 78424496T/G
3-4: 78606184T/G
3-4: 78684216G/A
3-4: 78712121A/G
3-4: 78712258C/T
3-4: 78855640A/G
3-4: 78890637G/A
3-4: 79013641G/A
3-4: 79077093A/C
3-4: 79126867C/T
3-4: 79143187G/T
3-4: 79159962C/T
3-4: 79161128A/G
3-4: 79163513G/C
3-4: 79174286C/T
3-4: 79301540C/G
3-4: 79429662T/C
3-4: 79521335C/T
3-4: 79613451G/A
3-4: 79647891G/A
3-4: 79865427A/G
3-4: 79873328T/G
3-4: 79940455C/A
3-4: 79996493A/G
3-4: 80066567C/T
3-4: 80103236C/A
3-4: 80189837C/T
3-4: 80214511C/T
3-4: 80313383T/A
3-4: 80329651G/A
3-4: 80359328A/G
3-4: 80708264A/T
3-4: 80760962G/A
3-4: 80881792A/G
3-4: 80907774A/G
3-4: 80978364T/A
3-4: 81007686T/A
3-4: 81069233C/A
3-4: 81099915A/G
3-4: 81155946C/T
3-4: 81241269A/C
3-4: 81415253T/G
3-4: 81793325T/C
3-4: 81956973A/G
3-4: 81958201A/T
3-4: 82028472G/A
3-4: 82101454T/C
4-1: 402486A/G
4-1: 665230G/C
4-1: 725964G/C
4-1: 969324A/G
4-1: 1062380G/A
4-1: 1113210C/T
4-1: 1292058G/A
4-1: 1330369T/C
4-1: 1514070C/A
4-1: 1657504A/T
4-1: 1846109A/T
4-1: 1856364T/A
4-1: 1860529C/T
4-1: 1867060C/T
4-1: 1890474C/T
4-1: 1895953G/A
4-1: 1900498C/G
4-1: 1901762C/T
4-1: 1901936C/T
4-1: 1910913C/T
4-1: 1927944A/G
4-1: 1950808C/A
4-1: 1992082T/C
4-1: 2059161G/A
4-1: 2235270G/A
4-1: 2350119G/A
4-1: 2528247A/G
4-1: 2528882T/C
4-1: 2569843C/T
4-1: 2688989A/G
4-1: 2833757A/T
4-1: 2859312C/T
4-1: 3016294G/A
4-1: 3023822T/G
4-1: 3056752C/T
4-1: 3243392T/A
4-1: 3362215G/C
4-1: 3439734G/A
4-1: 3475809C/A
4-1: 3515750C/T
4-1: 3605787A/G
4-1: 3656245C/A
4-1: 3681482T/C
4-1: 3839014A/G
4-1: 3935241A/G
4-1: 3953006A/C
4-1: 3962030C/T
4-1: 4017298T/G
4-1: 4072446C/G
4-1: 4105415G/C
4-1: 4140368A/G
4-1: 4186617C/T
4-1: 4200240C/A
4-1: 4214638T/A
4-1: 4224680A/T
4-1: 4297757C/A
4-1: 4328932A/G
4-1: 4357012G/A
4-1: 4387250A/G
4-1: 4454486T/C
4-1: 4537550A/T
4-1: 4627693G/A
4-1: 4749215T/G
4-1: 4758772C/A
4-1: 4764165T/C
4-1: 4800975C/A
4-1: 4806537C/T
4-1: 4832739C/G
4-1: 4862204A/T
4-1: 4862305C/T
4-1: 4874062T/A
4-1: 4902419C/A
4-1: 4939412G/A
4-1: 4941571C/T
4-1: 5027967T/A
4-1: 5030145G/A
4-1: 5054166A/T
4-1: 5106522C/A
4-1: 5122659A/G
4-1: 5216852T/A
4-1: 5226013G/A
4-1: 5240876T/A
4-1: 5289319G/A
4-1: 5303336C/G
4-1: 5305078C/T
4-1: 5305179A/T
4-1: 5305532G/A
4-1: 5305560C/T
4-1: 5319272T/C
4-1: 5337725A/T
4-1: 5379701G/A
4-1: 5390859T/C
4-1: 5408561A/G
4-1: 5499776T/C
4-1: 5509714A/T
4-1: 5566499A/G
4-1: 5652654T/A
4-1: 5727465A/G
4-1: 5743889G/A
4-1: 5750161A/G
4-1: 5766541G/A
4-1: 5799492C/T
4-1: 5810844A/G
4-1: 5824308C/T
4-1: 5842320T/C
4-1: 5843505T/C
4-1: 5843683A/G
4-1: 5853969G/T
4-1: 5911948A/G
4-1: 6036850C/T
4-1: 6037585G/A
4-1: 6041538A/G
4-1: 6043247C/G
4-1: 6085437G/A
4-1: 6137733A/G
4-1: 6197880C/T
4-1: 6255671A/C
4-1: 6285133G/A
4-1: 6311417A/G
4-1: 6317464A/G
4-1: 6374532A/C
4-1: 6408107A/G
4-1: 6481275C/A
4-1: 6490202T/C
4-1: 6522959A/G
4-1: 6528182T/A
4-1: 6536243T/G
4-1: 6560571T/A
4-1: 6578399T/C
4-1: 6582747C/T
4-1: 6589361G/T
4-1: 6599944A/T
4-1: 6601885A/C
4-1: 6614807T/C
4-1: 6664493G/A
4-1: 6693490G/A
4-1: 6733895T/A
4-1: 6788707A/T
4-1: 6822059T/G
4-1: 6875172T/C
4-1: 6939763C/T
4-1: 7002850G/A
4-1: 7065416T/A
4-1: 7141243G/A
4-1: 7183877T/C
4-1: 7214313A/G
4-1: 7239765A/G
4-1: 7290975T/C
4-1: 7305035T/C
4-1: 7311266T/C
4-1: 7352960C/T
4-1: 7379108G/A
4-1: 7405482G/C
4-1: 7449532C/T
4-1: 7561446G/C
4-1: 7568161A/T
4-1: 7568200G/A
4-1: 7568314A/G
4-1: 7568452T/C
4-1: 7568938A/G
4-1: 7569085A/G
4-1: 7569496G/A
4-1: 7569955A/G
4-1: 7570104C/T
4-1: 7571631A/C
4-1: 7608662C/G
4-1: 7900495C/A
4-1: 8034507G/A
4-1: 8055083A/G
4-1: 8071561G/A
4-1: 8110669G/A
4-1: 8113721A/C
4-1: 8114373C/G
4-1: 8115108T/C
4-1: 8115117A/G
4-1: 8115830C/T
4-1: 8125982A/T
4-1: 8126855A/G
4-1: 8224258T/C
4-1: 8236705A/T
4-1: 8257375T/C
4-1: 8260114A/G
4-1: 8267196T/C
4-1: 8267582T/C
4-1: 8267649T/A
4-1: 8271522A/C
4-1: 8274460A/G
4-1: 8274483G/A
4-1: 8276016A/G
4-1: 8293354T/C
4-1: 8311273T/C
4-1: 8385818A/G
4-1: 8426424T/A
4-1: 8426849G/A
4-1: 8494292C/A
4-1: 8556704T/C
4-1: 8633190T/C
4-1: 8633679A/G
4-1: 8633750A/G
4-1: 8634188A/G
4-1: 8634231G/C
4-1: 8634563G/A
4-1: 8635401T/C
4-1: 8694966A/G
4-1: 8832757C/A
4-1: 8846672G/C
4-1: 8902649A/G
4-1: 9120226C/T
4-1: 9152099G/A
4-1: 9194033C/T
4-1: 9441836G/C
4-1: 9479415T/A
4-1: 9508228C/T
4-1: 9508380A/G
4-1: 9508501T/C
4-1: 9535005A/T
4-1: 9535254T/C
4-1: 9550653A/T
4-1: 9645968G/A
4-1: 9701335G/T
4-1: 9701748T/C
4-1: 9726924G/T
4-1: 9819777G/A
4-1: 9832803T/G
4-1: 9847232C/A
4-1: 9874258T/C
4-1: 9884721G/T
4-1: 9885976T/C
4-1: 9906176A/G
4-1: 9921299C/A
4-1: 9921816C/T
4-1: 9932292G/A
4-1: 9970314A/G
4-1: 9980337C/T
4-1: 9993457T/G
4-1: 10042847G/A
4-1: 10056014G/A
4-1: 10140772T/C
4-1: 10183989G/A
4-1: 10232562A/C
4-1: 10248966G/T
4-1: 10280960G/A
4-1: 10297063C/T
4-1: 10307756G/A
4-1: 10353309T/A
4-1: 10364606C/T
4-1: 10410288T/C
4-1: 10452076A/G
4-1: 10504927A/G
4-1: 10519664T/C
4-1: 10531539T/G
4-1: 10537045C/T
4-1: 10558020A/T
4-1: 10574622A/T
4-1: 10589103C/A
4-1: 10626474C/T
4-1: 10670408A/G
4-1: 10710643G/A
4-1: 10764564C/T
4-1: 10908560T/G
4-1: 10922158T/A
4-1: 10963924G/A
4-1: 10979643T/A
4-1: 10995259G/A
4-1: 11005733T/A
4-1: 11016307C/T
4-1: 11039907A/T
4-1: 11057150A/C
4-1: 11075429T/G
4-1: 11086935A/G
4-1: 11107559G/T
4-1: 11128213G/A
4-1: 11185397G/A
4-1: 11195602C/T
4-1: 11222274T/C
4-1: 11241617T/G
4-1: 11242648T/C
4-1: 11289498A/T
4-1: 11305680A/C
4-1: 11305731G/A
4-1: 11319497G/A
4-1: 11415259C/T
4-1: 11417470C/T
4-1: 11417640T/C
4-1: 11417785A/T
4-1: 11417819A/C
4-1: 11417974T/G
4-1: 11418418G/A
4-1: 11418599T/C
4-1: 11418711A/T
4-1: 11418769G/A
4-1: 11465756T/C
4-1: 11549989G/A
4-1: 11573172T/C
4-1: 11573384G/C
4-1: 11573390G/A
4-1: 11639886C/T
4-1: 11650947T/A
4-1: 11719459C/T
4-1: 11745003G/A
4-1: 11789425G/C
4-1: 11817114G/A
4-1: 11828021T/C
4-1: 11845448A/T
4-1: 11866841T/G
4-1: 11902644T/C
4-1: 11908612C/T
4-1: 11925848T/A
4-1: 11994287T/A
4-1: 11997430C/T
4-1: 11997500T/C
4-1: 11998172C/G
4-1: 12004580A/G
4-1: 12118738C/T
4-1: 12168642G/A
4-1: 12168698G/A
4-1: 12168820C/T
4-1: 12169330A/G
4-1: 12169551A/G
4-1: 12169706T/A
4-1: 12169763T/C
4-1: 12170631A/G
4-1: 12173357G/C
4-1: 12178822A/G
4-1: 12178939C/G
4-1: 12225856A/G
4-1: 12270631G/A
4-1: 12323709A/C
4-1: 12342802T/G
4-1: 12378895T/G
4-1: 12458075T/A
4-1: 12538467T/C
4-1: 12584030G/C
4-1: 12595414C/A
4-1: 12694509C/A
4-1: 12723487C/T
4-1: 12760112C/T
4-1: 12790062A/G
4-1: 12821452G/A
4-1: 12832849T/C
4-1: 12916129T/A
4-1: 12929634T/A
4-1: 12969467C/T
4-1: 12974357C/T
4-1: 13055343C/T
4-1: 13101034C/T
4-1: 13143744C/T
4-1: 13225281A/G
4-1: 13382666C/T
4-1: 13390949G/T
4-1: 13426344T/C
4-1: 13443356A/C
4-1: 13461215A/C
4-1: 13463994G/A
4-1: 13512422C/T
4-1: 13569100T/C
4-1: 13631509T/C
4-1: 13632639T/A
4-1: 13632664C/T
4-1: 13662816G/A
4-1: 13704783C/A
4-1: 13725809C/A
4-1: 13752818G/C
4-1: 13787055A/T
4-1: 13827782C/T
4-1: 13831299T/C
4-1: 13865286G/C
4-1: 13875472C/T
4-1: 13910676T/C
4-1: 13921911C/T
4-1: 13973651C/T
4-1: 13980297A/T
4-1: 14001609G/A
4-1: 14009756C/T
4-1: 14010533T/C
4-1: 14023856T/G
4-1: 14086510T/C
4-1: 14315219G/T
4-1: 14315226T/C
4-1: 14315240T/A
4-1: 14383104T/C
4-1: 14444262G/A
4-1: 14462059T/A
4-1: 14601356A/G
4-1: 14606884G/T
4-1: 14613489C/A
4-1: 14616715G/C
4-1: 14617094C/T
4-1: 14617185T/A
4-1: 14636276A/T
4-1: 14664368T/C
4-1: 14678275C/T
4-1: 14690240C/T
4-1: 14693486A/T
4-1: 14693949G/A
4-1: 14693960C/T
4-1: 14708530A/G
4-1: 14719454G/A
4-1: 14730086A/G
4-1: 14828785C/G
4-1: 14842552C/T
4-1: 14935180A/T
4-1: 14939564T/G
4-1: 14950149T/C
4-1: 14984472G/T
4-1: 14996479A/T
4-1: 15041699C/A
4-1: 15042491A/T
4-1: 15058905C/T
4-1: 15117222A/T
4-1: 15122028C/T
4-1: 15154070G/A
4-1: 15167230G/A
4-1: 15172458A/T
4-1: 15172650G/A
4-1: 15183886C/T
4-1: 15203818C/A
4-1: 15219609T/C
4-1: 15268246T/C
4-1: 15282860G/A
4-1: 15312752C/T
4-1: 15332847G/T
4-1: 15390577T/G
4-1: 15440741G/A
4-1: 15473684T/C
4-1: 15515291C/A
4-1: 15542474G/A
4-1: 15571571T/C
4-1: 15585594T/A
4-1: 15588701G/A
4-1: 15620053T/A
4-1: 15635735G/A
4-1: 15644293T/C
4-1: 15647652G/A
4-1: 15686010T/C
4-1: 15719947C/T
4-1: 15779377C/T
4-1: 15791399G/C
4-1: 15901947T/A
4-1: 15947367A/T
4-1: 15985568T/A
4-1: 16013386C/T
4-1: 16035459G/T
4-1: 16057205C/A
4-1: 16210500T/A
4-1: 16291003C/T
4-1: 16324613C/A
4-1: 16373907C/T
4-1: 16400644A/C
4-1: 16400650A/G
4-1: 16404891C/T
4-1: 16412719G/A
4-1: 16412960A/G
4-1: 16424139C/T
4-1: 16437503C/T
4-1: 16456490C/A
4-1: 16507482G/A
4-1: 16571109A/T
4-1: 16572738T/C
4-1: 16609581A/T
4-1: 16615629A/G
4-1: 16662448C/T
4-1: 16674187A/G
4-1: 16704591C/T
4-1: 16734998C/G
4-1: 16761962T/C
4-1: 16794136G/T
4-1: 16849207C/T
4-1: 16881030T/C
4-1: 16960732C/T
4-1: 16983126A/C
4-1: 16990610T/C
4-1: 17057306A/G
4-1: 17089516A/C
4-1: 17100963G/C
4-1: 17119882T/A
4-1: 17305077G/A
4-1: 17378364C/.
4-1: 17380033G/A
4-1: 17399258C/T
4-1: 17449708G/C
4-1: 17484502C/A
4-1: 17534842A/C
4-1: 17587728G/A
4-1: 17598860C/T
4-1: 17599256T/G
4-1: 17732084G/A
4-1: 17829193G/C
4-1: 17852944A/T
4-1: 17927259C/T
4-1: 17940003A/T
4-1: 17940193C/A
4-1: 17955852T/C
4-1: 18045037T/A
4-1: 18070425C/A
4-1: 18075536T/A
4-1: 18076053T/C
4-1: 18078852T/A
4-1: 18090557G/A
4-1: 18111743A/G
4-1: 18141487C/T
4-1: 18154920C/T
4-1: 18293890A/T
4-1: 18348976T/C
4-1: 18369458G/A
4-1: 18383934C/T
4-1: 18399605A/C
4-1: 18425499T/A
4-1: 18466619C/T
4-1: 18504468G/C
4-1: 18550140C/A
4-1: 18569979G/A
4-1: 18594447G/A
4-1: 18610504A/G
4-1: 18626367C/T
4-1: 18635049G/A
4-1: 18648783A/G
4-1: 18710829A/T
4-1: 18761465A/T
4-1: 18788491C/A
4-1: 18813692T/C
4-1: 18847434G/A
4-1: 18920845G/A
4-1: 19002258A/T
4-1: 19017497C/G
4-1: 19033181C/T
4-1: 19062637C/A
4-1: 19091556T/C
4-1: 19108617A/G
4-1: 19293645T/A
4-1: 19414211T/C
4-1: 19501641G/T
4-1: 19564681T/C
4-1: 19651628C/A
4-1: 19663745A/G
4-1: 19692524T/A
4-1: 19710048G/C
4-1: 19949534C/T
4-1: 20011194G/C
4-1: 20092226G/A
4-1: 20135496G/A
4-1: 20256211T/C
4-1: 20284406A/G
4-1: 20302132G/A
4-1: 20322218T/C
4-1: 20367865G/T
4-1: 20483156T/C
4-1: 20484762A/G
4-1: 20622569C/T
4-1: 20640039T/G
4-1: 20647026T/A
4-1: 20649560T/G
4-1: 20663687T/A
4-1: 20702718G/A
4-1: 20731183A/C
4-1: 20754957G/A
4-1: 20766873A/T
4-1: 20806862G/C
4-1: 20809586G/A
4-1: 20816844T/C
4-1: 20842983C/G
4-1: 20862622C/T
4-1: 20890234A/C
4-1: 20945612C/A
4-1: 20957986A/C
4-1: 20971799C/T
4-1: 21024532C/T
4-1: 21036082A/G
4-1: 21043180C/A
4-1: 21068099G/T
4-1: 21137094A/C
4-1: 21186135C/T
4-1: 21239889T/A
4-1: 21251242C/T
4-1: 21308370T/C
4-1: 21329488G/T
4-1: 21362618A/C
4-1: 21522295A/T
4-1: 21578817T/A
4-1: 21616316T/C
4-1: 21683542C/G
4-1: 21805569A/G
4-1: 22081294C/G
4-1: 22094081C/T
4-1: 22107396C/A
4-1: 22135632C/A
4-1: 22146771A/G
4-1: 22279563A/G
4-1: 22293186C/G
4-1: 22323461C/A
4-1: 22343929G/A
4-1: 22354697A/G
4-1: 22365669C/G
4-1: 22376785G/A
4-1: 22430534T/G
4-1: 22519521T/C
4-1: 22532848A/G
4-1: 22564755C/T
4-1: 22610833T/C
4-1: 22644931T/C
4-1: 22718203T/C
4-1: 22747140C/G
4-1: 22803337G/A
4-1: 22835394T/C
4-1: 22863798G/T
4-1: 22895608G/A
4-1: 22908080C/G
4-1: 22920542T/C
4-1: 22930936A/G
4-1: 22938056A/C
4-1: 22938072G/T
4-1: 22939003T/C
4-1: 22939069T/G
4-1: 22939172G/A
4-1: 22939269T/A
4-1: 22939411G/A
4-1: 22939454G/T
4-1: 22939580G/A
4-1: 22939678G/A
4-1: 22939878G/T
4-1: 22953723A/G
4-1: 22964943G/C
4-1: 23006853T/G
4-1: 23040536A/G
4-1: 23040681G/A
4-1: 23217222A/C
4-1: 23228492T/A
4-1: 23240838A/G
4-1: 23258553C/T
4-1: 23273849G/A
4-1: 23276639G/A
4-1: 23284798A/G
4-1: 23312174C/A
4-1: 23323243G/A
4-1: 23350355T/C
4-1: 23361657A/G
4-1: 23396991T/C
4-1: 23397946C/T
4-1: 23412507C/T
4-1: 23444086T/G
4-1: 23461734C/G
4-1: 23475578T/C
4-1: 23504699T/A
4-1: 23551553G/C
4-1: 23580268C/A
4-1: 23590498G/A
4-1: 23629368A/G
4-1: 23681385T/C
4-1: 23728424C/T
4-1: 23778590A/G
4-1: 23843379A/G
4-1: 23935348T/C
4-1: 23946380T/G
4-1: 23974721A/G
4-1: 24006351G/A
4-1: 24137674C/A
4-1: 24254501G/A
4-1: 24258467T/A
4-1: 24309943A/G
4-1: 24359308G/A
4-1: 24359767G/T
4-1: 24379819G/A
4-1: 24442264G/A
4-1: 24483609G/T
4-1: 24571464A/T
4-1: 24611508T/C
4-1: 24676321C/T
4-1: 24772131A/G
4-1: 24795275G/A
4-1: 24856262G/A
4-1: 24856296T/A
4-1: 24856308T/A
4-1: 25297409T/C
4-1: 25317743T/G
4-1: 25424288T/C
4-1: 25427990T/A
4-1: 25428598A/G
4-1: 25428823T/C
4-1: 25431291A/C
4-1: 25431481T/G
4-1: 25431499G/C
4-1: 25431595T/C
4-1: 25442740A/C
4-1: 25479223T/A
4-1: 25514922T/A
4-1: 25543475A/T
4-1: 25688830C/T
4-1: 25712527T/A
4-1: 25726679C/G
4-1: 25902194G/A
4-1: 25963759A/G
4-1: 25985325T/C
4-1: 26012725G/A
4-1: 26012878T/A
4-1: 26012902C/G
4-1: 26012916C/T
4-1: 26012923A/G
4-1: 26012964C/T
4-1: 26128804G/C
4-1: 26258494T/C
4-1: 26269299G/C
4-1: 26334788C/A
4-1: 26361790C/G
4-1: 26375538G/A
4-1: 26628643T/A
4-1: 26736781C/G
4-1: 26779895T/G
4-1: 26842424T/A
4-1: 26885508A/T
4-1: 26931898T/G
4-1: 27036199C/G
4-1: 27041308T/C
4-1: 27079718A/G
4-1: 27110961G/A
4-1: 27159484G/A
4-1: 27173919G/A
4-1: 27174058A/G
4-1: 27186635A/C
4-1: 27254580T/G
4-1: 27264600A/T
4-1: 27282838A/T
4-1: 27294797T/A
4-1: 27322580A/G
4-1: 27335219A/C
4-1: 27360460C/T
4-1: 27432373G/A
4-1: 27444269C/T
4-1: 27464251G/A
4-1: 27567246A/T
4-1: 27613118T/C
4-1: 27658669C/T
4-1: 27696271G/C
4-1: 27725301G/T
4-1: 28050775A/G
4-1: 28052811T/C
4-1: 28071285C/T
4-1: 28072382A/G
4-1: 28091430G/T
4-1: 28118082G/A
4-1: 28130986C/T
4-1: 28156819G/A
4-1: 28167025A/T
4-1: 28167806T/A
4-1: 28167822T/A
4-1: 28168112G/A
4-1: 28168219A/T
4-1: 28168426T/A
4-1: 28168726G/A
4-1: 28168831A/G
4-1: 28168890G/A
4-1: 28169003C/T
4-1: 28171146C/T
4-1: 28171360T/C
4-1: 28171408A/G
4-1: 28171463T/A
4-1: 28171629G/A
4-1: 28172758G/A
4-1: 28172857G/A
4-1: 28173121C/T
4-1: 28173364G/A
4-1: 28173413C/T
4-1: 28173423C/T
4-1: 28173615T/C
4-1: 28185616T/C
4-1: 28233169C/T
4-1: 28254177G/T
4-1: 28301566C/A
4-1: 28366294T/A
4-1: 28367366A/T
4-1: 28394265C/T
4-1: 28448318G/A
4-1: 28490097C/T
4-1: 28563644T/G
4-1: 28601681T/G
4-1: 28617047T/C
4-1: 28733329G/T
4-1: 28740495C/T
4-1: 28775101C/A
4-1: 28867451G/A
4-1: 28906069G/T
4-1: 29029783C/T
4-1: 29060776A/T
4-1: 29089727C/T
4-1: 29140616C/A
4-1: 29187768A/G
4-1: 29251970T/A
4-1: 29355461G/A
4-1: 29389169T/C
4-1: 29433210G/T
4-1: 29456950T/C
4-1: 29494930C/A
4-1: 29542089T/G
4-1: 29544048A/T
4-1: 29554458G/A
4-1: 29654089C/T
4-1: 29673809C/T
4-1: 29673854C/T
4-1: 29678346C/A
4-1: 29704699T/A
4-1: 29849082T/A
4-1: 29856804A/.
4-1: 29858880A/G
4-1: 29860041A/G
4-1: 29875485T/A
4-1: 30021171G/T
4-1: 30080360A/G
4-1: 30143240A/G
4-1: 30188582G/A
4-1: 30198763C/T
4-1: 30238086T/C
4-1: 30264674C/T
4-1: 30291623T/C
4-1: 30304613A/G
4-1: 30426398T/C
4-1: 30467726G/A
4-1: 30479251G/A
4-1: 30501026C/T
4-1: 30511329T/C
4-1: 30535621G/T
4-1: 30554600A/T
4-1: 30568558T/A
4-1: 30623725G/A
4-1: 30681528A/T
4-1: 30729591T/C
4-1: 30744519C/T
4-1: 30796896A/G
4-1: 30883761C/T
4-1: 30945421C/T
4-1: 30985351T/A
4-1: 31143534G/A
4-1: 31144736T/C
4-1: 31190825C/G
4-1: 31204129G/A
4-1: 31214869C/T
4-1: 31229535A/T
4-1: 31230153A/G
4-1: 31233328T/G
4-1: 31233688C/T
4-1: 31233697C/T
4-1: 31233698G/A
4-1: 31233820C/T
4-1: 31233821G/A
4-1: 31233825G/.
4-1: 31233830T/C
4-1: 31233851T/G
4-1: 31233888T/G
4-1: 31233890A/T
4-1: 31233915T/C
4-1: 31234033C/T
4-1: 31234087T/C
4-1: 31234222A/G
4-1: 31234336G/A
4-1: 31234358A/C
4-1: 31234384C/A
4-1: 31234393G/A
4-1: 31245080G/A
4-1: 31256595C/T
4-1: 31299134G/A
4-1: 31375495A/T
4-1: 31438221C/T
4-1: 31450737C/T
4-1: 31471834A/G
4-1: 31490111A/C
4-1: 31511177T/C
4-1: 31534858C/T
4-1: 31553095G/A
4-1: 31575651C/T
4-1: 31623494T/A
4-1: 31634150G/A
4-1: 31654608A/G
4-1: 31727595A/T
4-1: 31830427T/C
4-1: 31952734C/A
4-1: 32034680T/C
4-1: 32036532T/A
4-1: 32040427T/G
4-1: 32056732A/G
4-1: 32124364G/T
4-1: 32202447G/A
4-1: 32203860T/C
4-1: 32249834A/G
4-1: 32277159A/G
4-1: 32279470A/C
4-1: 32328660T/C
4-1: 32363780G/T
4-1: 32434426A/T
4-1: 32447140A/G
4-1: 32475397C/T
4-1: 32574668A/C
4-1: 32590421A/G
4-1: 32734775G/A
4-1: 32750223C/T
4-1: 32763673T/A
4-1: 32797589A/T
4-1: 32839913A/T
4-1: 32863271T/A
4-1: 32904084G/A
4-1: 32983595C/T
4-1: 33076060T/C
4-1: 33088496A/T
4-1: 33204552T/A
4-1: 33237280A/C
4-1: 33333654A/C
4-1: 33397100C/T
4-1: 33475249G/A
4-1: 33515220A/G
4-1: 33521415A/G
4-1: 33586844C/A
4-1: 33605525C/A
4-1: 33694677T/G
4-1: 33711201A/T
4-1: 33777808G/A
4-1: 33899090T/A
4-1: 33900272G/T
4-1: 33912589C/T
4-1: 33937350C/T
4-1: 33943910T/A
4-1: 33948097G/C
4-1: 33994582T/C
4-1: 34059681T/A
4-1: 34101870T/C
4-1: 34133828C/T
4-1: 34186278C/A
4-1: 34263179T/A
4-1: 34312424A/C
4-1: 34332100G/A
4-1: 34347484T/G
4-1: 34371088G/A
4-1: 34371316C/A
4-1: 34385925G/A
4-1: 34432125C/T
4-1: 34442828A/G
4-1: 34460987G/A
4-1: 34482222A/G
4-1: 34482241C/T
4-1: 34482607C/T
4-1: 34544217T/C
4-1: 34597805G/A
4-1: 34839140G/A
4-1: 34839865G/A
4-1: 34916891A/G
4-1: 34964880A/T
4-1: 35033645A/C
4-1: 35082899A/T
4-1: 35083129A/G
4-1: 35107976C/G
4-1: 35126333G/C
4-1: 35154285G/T
4-1: 35195233C/T
4-1: 35257693C/A
4-1: 35285143G/T
4-1: 35299089C/T
4-1: 35342207C/A
4-1: 35393328C/G
4-1: 35437060T/C
4-1: 35462566A/G
4-1: 35580841A/G
4-1: 35590391G/T
4-1: 35591186C/T
4-1: 35591857C/A
4-1: 35591882A/G
4-1: 35604973T/A
4-1: 35744053A/C
4-1: 35817451A/G
4-1: 35855633T/G
4-1: 35933346A/T
4-1: 36040893G/T
4-1: 36053001T/C
4-1: 36165942A/G
4-1: 36179841G/T
4-1: 36194019C/T
4-1: 36235497T/G
4-1: 36259972A/G
4-1: 36278192G/T
4-1: 36278498C/T
4-1: 36278726C/T
4-1: 36278857C/T
4-1: 36279435A/C
4-1: 36279896C/A
4-1: 36279973C/T
4-1: 36280110A/G
4-1: 36280707G/T
4-1: 36280840C/T
4-1: 36306493A/G
4-1: 36333241G/A
4-1: 36412352C/T
4-1: 36433636A/T
4-1: 36434291T/A
4-1: 36434519G/T
4-1: 36446432C/T
4-1: 36465721T/C
4-1: 36507656A/T
4-1: 36542047T/C
4-1: 36554191C/A
4-1: 36573241G/A
4-1: 36597671T/C
4-1: 36637860T/G
4-1: 36681132A/G
4-1: 36719903G/A
4-1: 36753317A/G
4-1: 36753656G/A
4-1: 36849765T/G
4-1: 36879911A/C
4-1: 36961282G/A
4-1: 36974042T/G
4-1: 37064927C/T
4-1: 37101587G/A
4-1: 37120051C/A
4-1: 37130608C/T
4-1: 37189821C/G
4-1: 37189957C/A
4-1: 37190267G/A
4-1: 37243649G/A
4-1: 37286423T/A
4-1: 37293392T/C
4-1: 37309339T/C
4-1: 37356834C/T
4-1: 37434227G/A
4-1: 37476698G/T
4-1: 37506696T/A
4-1: 37509146T/C
4-1: 37522881T/C
4-1: 37523643G/C
4-1: 37523852C/T
4-1: 37543574T/C
4-1: 37563139C/A
4-1: 37603080C/T
4-1: 37649049C/G
4-1: 37679724G/C
4-1: 37707917A/G
4-1: 37722382A/G
4-1: 37725027G/A
4-1: 37735713C/A
4-1: 37756686T/C
4-1: 37791212C/A
4-1: 37828633A/G
4-1: 37896461T/C
4-1: 37932104G/A
4-1: 37959471C/T
4-1: 37970158C/T
4-1: 37972872C/A
4-1: 37974172A/G
4-1: 37974433G/A
4-1: 37974954T/G
4-1: 37980774C/T
4-1: 37980828C/T
4-1: 37981271C/T
4-1: 37992322A/T
4-1: 38027085G/T
4-1: 38038562G/A
4-1: 38054183C/A
4-1: 38069884A/G
4-1: 38118535C/A
4-1: 38147749G/T
4-1: 38179849T/G
4-1: 38198154C/T
4-1: 38208731T/C
4-1: 38216056G/A
4-1: 38216134C/T
4-1: 38227168A/G
4-1: 38236069C/T
4-1: 38236188G/A
4-1: 38237784A/G
4-1: 38237902G/A
4-1: 38284063T/A
4-1: 38287284C/T
4-1: 38287330C/T
4-1: 38287864T/A
4-1: 38287897G/T
4-1: 38288783G/C
4-1: 38295656G/A
4-1: 38326058T/C
4-1: 38423644A/T
4-1: 38428660T/C
4-1: 38477000G/C
4-1: 38488183T/G
4-1: 38499470T/G
4-1: 38510258G/A
4-1: 38547519T/A
4-1: 38549252T/G
4-1: 38555351G/A
4-1: 38555461T/A
4-1: 38557783A/G
4-1: 38558341C/A
4-1: 38586790G/C
4-1: 38589854C/T
4-1: 38590270G/A
4-1: 38593531G/T
4-1: 38593826G/A
4-1: 38597681C/T
4-1: 38731761T/C
4-1: 38869142T/A
4-1: 38879518A/T
4-1: 38881774A/C
4-1: 38918484C/T
4-1: 38931246T/C
4-1: 38941454C/T
4-1: 38943333A/T
4-1: 38950449A/G
4-1: 38960003G/A
4-1: 38981867G/C
4-1: 38983250C/A
4-1: 38985354C/T
4-1: 38993214T/A
4-1: 39026844C/A
4-1: 39051929A/G
4-1: 39077838C/T
4-1: 39114669A/G
4-1: 39157887T/C
4-1: 39165286A/G
4-1: 39182427T/C
4-1: 39208185T/C
4-1: 39246445T/C
4-1: 39246474G/A
4-1: 39424128T/C
4-1: 39461295A/T
4-1: 39528135T/G
4-1: 39529732A/T
4-1: 39559915T/A
4-1: 39597791A/G
4-1: 39706237C/T
4-1: 39716767C/A
4-1: 39717794T/C
4-1: 39718412A/C
4-1: 39718780G/T
4-1: 39719574A/T
4-1: 39732172A/G
4-1: 39732450G/A
4-1: 39732455C/T
4-1: 39746088G/A
4-1: 39754314A/G
4-1: 39757148T/C
4-1: 39771083G/T
4-1: 39773392A/T
4-1: 39836296C/T
4-1: 39861666C/A
4-1: 39866368C/T
4-1: 39879275T/G
4-1: 39904965A/C
4-1: 39936507C/T
4-1: 39939872A/T
4-1: 39964831G/T
4-1: 40036821A/G
4-1: 40038390G/A
4-1: 40097924A/T
4-1: 40122637G/A
4-1: 40171265T/C
4-1: 40208652G/A
4-1: 40253494T/C
4-1: 40299940T/C
4-1: 40352011C/T
4-1: 40400907A/C
4-1: 40480836A/T
4-1: 40498843A/G
4-1: 40525440T/A
4-1: 40531254A/G
4-1: 40533333G/T
4-1: 40533573G/T
4-1: 40533804G/A
4-1: 40534009A/G
4-1: 40534235T/A
4-1: 40545469G/A
4-1: 40613282C/T
4-1: 40672486G/A
4-1: 40726733G/A
4-1: 40761560G/T
4-1: 40795901A/C
4-1: 40821517C/A
4-1: 40866702T/C
4-1: 40931620C/G
4-1: 40980836C/T
4-1: 41009277G/A
4-1: 41037266T/C
4-1: 41063201C/T
4-1: 41108464G/A
4-1: 41140146A/G
4-1: 41199183T/C
4-1: 41249093G/C
4-1: 41279665T/G
4-1: 41349845C/T
4-1: 41535268C/A
4-1: 41596964C/T
4-1: 41623830A/G
4-1: 41687483G/C
4-1: 41717117T/C
4-1: 41757644C/A
4-1: 41827661T/A
4-1: 41869422T/A
4-1: 41934272T/A
4-1: 41946897C/T
4-1: 41975101C/T
4-1: 42048801T/A
4-1: 42062502C/G
4-1: 42073801C/T
4-1: 42095467G/T
4-1: 42097970A/G
4-1: 42138139G/A
4-1: 42260092C/T
4-1: 42291962T/C
4-1: 42326103A/T
4-1: 42361713T/C
4-1: 42395481A/G
4-1: 42398072G/A
4-1: 42409506A/G
4-1: 42439661T/C
4-1: 42440247T/C
4-1: 42488793T/C
4-1: 42494806C/T
4-1: 42532778T/A
4-1: 42563711T/G
4-1: 42568581T/C
4-1: 42624661G/A
4-1: 42657811G/T
4-1: 42709818A/G
4-1: 42750366A/G
4-1: 42800874G/A
4-1: 42834517T/C
4-1: 42882727T/C
4-1: 42916411A/C
4-1: 42938866G/T
4-1: 42951519A/G
4-1: 42995893C/T
4-1: 43044838A/G
4-1: 43091553C/G
4-1: 43102647C/T
4-1: 43128226G/A
4-1: 43138748A/G
4-1: 43186367C/T
4-1: 43236464G/A
4-1: 43301325C/T
4-1: 43423006T/C
4-1: 43513626C/G
4-1: 43589991C/T
4-1: 43652598T/C
4-1: 43695748C/T
4-1: 43717519C/T
4-1: 43753179A/T
4-1: 43784466T/A
4-1: 43810368T/G
4-1: 43815573T/A
4-1: 43856149C/A
4-1: 43919717T/A
4-1: 43964933C/A
4-1: 43992162G/C
4-1: 44078467T/C
4-1: 44132712G/A
4-1: 44207816C/A
4-1: 44284192C/A
4-1: 44313543C/A
4-1: 44334368A/G
4-1: 44378865C/A
4-1: 44424107A/G
4-1: 44472096C/T
4-1: 44568538T/C
4-1: 44611325G/A
4-1: 44647536T/G
4-1: 44703142G/T
4-1: 44733961A/G
4-1: 44762595T/C
4-1: 44824762A/T
4-1: 44884065G/A
4-1: 44908840G/A
4-1: 44921899A/T
4-1: 44932886C/T
4-1: 44947513G/A
4-1: 44952935C/A
4-1: 44953006T/C
4-1: 44955035A/T
4-1: 44958605T/A
4-1: 44958634G/A
4-1: 44958697T/C
4-1: 44992117A/.
4-1: 44996855A/G
4-1: 45038963C/A
4-1: 45077292A/G
4-1: 45081927A/C
4-1: 45089698G/T
4-1: 45135967C/T
4-1: 45139466T/A
4-1: 45139615A/T
4-1: 45159833G/A
4-1: 45245604G/A
4-1: 45343554C/T
4-1: 45350657A/G
4-1: 45362838A/G
4-1: 45397004C/T
4-1: 45397455G/C
4-1: 45397501C/G
4-1: 45413252C/T
4-1: 45432630T/C
4-1: 45462073A/G
4-1: 45492399T/C
4-1: 45727453A/G
4-1: 45728693A/T
4-1: 45742762A/T
4-1: 45765276T/G
4-1: 45908720G/T
4-1: 45909911A/G
4-1: 45948126A/C
4-1: 46006761A/G
4-1: 46075872T/A
4-1: 46147815C/T
4-1: 46184323A/G
4-1: 46204873A/G
4-1: 46264704G/A
4-1: 46295012T/C
4-1: 46300325A/G
4-1: 46331352T/C
4-1: 46353195A/G
4-1: 46362139A/G
4-1: 46362219A/G
4-1: 46362528C/T
4-1: 46362801C/T
4-1: 46363114G/C
4-1: 46363215C/G
4-1: 46364661T/C
4-1: 46365559T/G
4-1: 46403985C/T
4-1: 46454577C/T
4-1: 46465240A/C
4-1: 46543394G/A
4-1: 46577508T/A
4-1: 46646305A/G
4-1: 46732406T/C
4-1: 46770490A/G
4-1: 46856571C/T
4-1: 46933025C/T
4-1: 47029906C/T
4-1: 47146662T/G
4-1: 47235382G/A
4-1: 47537510T/C
4-1: 47599070C/T
4-1: 47609109C/T
4-1: 47646606T/C
4-1: 47646711G/A
4-1: 47646827A/G
4-1: 47646943A/G
4-1: 47647598T/G
4-1: 47647599G/T
4-1: 47647716T/C
4-1: 47661335T/A
4-1: 47676466T/A
4-1: 47690411A/G
4-1: 47725149A/G
4-1: 47754394T/C
4-1: 47795021A/G
4-1: 47827783G/A
4-1: 47838847A/G
4-1: 47863117T/C
4-1: 47901690G/T
4-1: 47945118T/G
4-1: 47978819T/C
4-1: 48004535T/A
4-1: 48027459C/T
4-1: 48086559G/C
4-1: 48128807C/T
4-1: 48196943G/T
4-1: 48207723C/G
4-1: 48312725A/G
4-1: 48315882G/A
4-1: 48328202A/G
4-1: 48469221G/A
4-1: 48501038A/G
4-1: 48540916T/A
4-1: 48595475A/G
4-1: 48635980T/C
4-1: 48638408A/G
4-1: 48679417A/G
4-1: 48713500G/A
4-1: 48959523T/G
4-1: 49201950T/C
4-1: 49220812G/A
4-1: 49234802A/T
4-1: 49282137A/G
4-1: 49328203G/A
4-1: 49329450A/G
4-1: 49330046T/C
4-1: 49332105A/G
4-1: 49332624A/G
4-1: 49334330C/T
4-1: 49338341A/T
4-1: 49353416G/A
4-1: 49380054T/G
4-1: 49412108G/A
4-1: 49456710T/G
4-1: 49475277G/A
4-1: 49494278A/C
4-1: 49516607C/T
4-1: 49536560C/G
4-1: 49547154T/A
4-1: 49597255A/G
4-1: 49625514C/T
4-1: 49664827C/T
4-1: 49773011A/T
4-1: 49818466G/A
4-1: 49821747C/T
4-1: 49847626A/C
4-1: 49890089A/G
4-1: 49938818A/G
4-1: 49982588C/A
4-1: 50003791A/C
4-1: 50015918C/A
4-1: 50030129C/T
4-1: 50047193T/C
4-1: 50089148A/T
4-1: 50116810G/A
4-1: 50150756G/A
4-1: 50190774C/T
4-1: 50288041T/C
4-1: 50487837G/T
4-1: 50642835C/T
4-1: 50654106A/G
4-1: 50743821T/G
4-1: 50823543A/T
4-1: 50897418T/C
4-1: 50936768T/C
4-1: 50993534G/A
4-1: 51124314C/T
4-1: 51183457A/G
4-1: 51280162G/T
4-1: 51302206G/C
4-1: 51320162C/T
4-1: 51395174T/G
4-1: 51431087G/A
4-1: 51450645C/T
4-1: 51482572T/C
4-1: 51503613A/G
4-1: 51531686T/C
4-1: 51584440T/A
4-1: 51595448A/C
4-1: 51751105G/A
4-1: 51769944G/A
4-1: 51815327T/C
4-1: 51869750C/A
4-1: 51892571A/G
4-1: 51959294T/G
4-1: 52018084T/C
4-1: 52085466A/C
4-1: 52250376G/A
4-1: 52319959C/G
4-1: 52331594A/G
4-1: 52335683A/G
4-1: 52347312A/T
4-1: 52453470C/A
4-1: 52481587C/A
4-1: 52535947C/A
4-1: 52624325G/A
4-1: 52740388C/T
4-1: 52775307C/T
4-1: 52880949C/T
4-1: 52889369T/A
4-1: 52920923G/A
4-1: 52993715A/T
4-1: 53043401C/T
4-1: 53057753A/C
4-1: 53207734C/T
4-1: 53258997T/G
4-1: 53311607C/T
4-1: 53324547C/T
4-1: 53438765T/C
4-1: 53512048A/G
4-1: 53574487T/C
4-1: 53640318C/A
4-1: 53699954T/C
4-1: 53730183C/T
4-1: 53787473A/C
4-1: 53834656C/T
4-1: 53883955G/A
4-1: 53926720G/A
4-1: 53977533T/C
4-1: 53992786A/G
4-1: 54005842C/T
4-1: 54034087C/A
4-1: 54110933G/A
4-1: 54140287A/T
4-1: 54208298T/C
4-1: 54218697C/T
4-1: 54275868G/A
4-1: 54307187C/T
4-1: 54340967G/A
4-1: 54411007T/G
4-1: 54442032A/G
4-1: 54461967G/A
4-1: 54606526C/T
4-1: 54669181C/T
4-1: 54719817A/G
4-1: 54721834A/T
4-1: 54812828G/A
4-1: 54902438T/C
4-1: 55011996A/C
4-1: 55054138A/G
4-1: 55203341C/T
4-1: 55339986T/G
4-1: 55388111A/G
4-1: 55422799C/T
4-1: 55434532G/A
4-1: 55447645A/G
4-1: 55456326G/C
4-1: 55471431T/G
4-1: 55472191C/T
4-1: 55484183A/G
4-1: 55500176T/A
4-1: 55531941A/G
4-1: 55532879A/T
4-1: 55533048C/T
4-1: 55546171T/G
4-1: 55572067C/A
4-1: 55602244A/G
4-1: 55622043G/A
4-1: 55622154G/A
4-1: 55623617A/T
4-1: 55624743C/T
4-1: 55624791C/A
4-1: 55624821C/T
4-1: 55625149C/T
4-1: 55655947G/A
4-1: 55666746A/G
4-1: 55739261T/A
4-1: 55790180G/A
4-1: 55824374C/T
4-1: 55824679G/A
4-1: 55945416T/A
4-1: 56011800G/A
4-1: 56156840G/A
4-1: 56232613T/C
4-1: 56279957T/G
4-1: 56351903T/A
4-1: 56392688G/A
4-1: 56481432G/C
4-1: 56585867T/C
4-1: 56643317C/T
4-1: 56684155T/C
4-1: 56757222A/T
4-1: 56757483A/G
4-1: 56814620G/T
4-1: 56882336T/C
4-1: 56903403T/A
4-1: 57031151A/C
4-1: 57060816A/T
4-1: 57095558G/A
4-1: 57172997G/T
4-1: 57219214G/T
4-1: 57247209G/C
4-1: 57274176C/T
4-1: 57454531C/T
4-1: 57520305T/C
4-1: 57544942C/T
4-1: 57587649T/C
4-1: 57787262G/A
4-1: 57923586C/T
4-1: 57951682C/T
4-1: 57968784C/T
4-1: 57989843A/T
4-1: 58033579A/C
4-1: 58080660A/T
4-1: 58123056G/A
4-1: 58213148T/C
4-1: 58227905C/A
4-1: 58259048G/T
4-1: 58270682C/T
4-1: 58347152T/C
4-1: 58433525T/A
4-1: 58451379T/A
4-1: 58509116T/C
4-1: 58519969G/A
4-1: 58520519T/C
4-1: 58618809A/G
4-1: 58661889A/C
4-1: 58794988T/C
4-1: 58812188G/C
4-1: 58824172G/A
4-1: 58898002T/C
4-1: 59018504G/C
4-1: 59027207G/A
4-1: 59123560G/A
4-1: 59180374G/A
4-1: 59319990C/A
4-1: 59340299A/T
4-1: 59449189T/A
4-1: 59460521A/T
4-1: 59525942C/A
4-1: 59571621T/C
4-1: 59581549C/T
4-1: 59642622A/T
4-1: 59738092A/G
4-1: 59768405T/A
4-1: 59805327G/T
4-1: 59836231G/A
4-1: 59842932C/T
4-1: 59843036C/T
4-1: 59843085G/A
4-1: 59939911C/T
4-1: 59960487T/C
4-1: 59961010C/G
4-1: 59961225G/A
4-1: 59988129T/C
4-1: 60017345G/A
4-1: 60067138G/A
4-1: 60102314G/A
4-1: 60121614T/G
4-1: 60239915C/T
4-1: 60244285A/T
4-1: 60285066C/T
4-1: 60314062G/A
4-1: 60345501T/G
4-1: 60372653G/A
4-1: 60387660T/A
4-1: 60412154G/A
4-1: 60484235C/A
4-1: 60497628A/C
4-1: 60515077T/C
4-1: 60582466T/A
4-1: 60593162C/T
4-1: 60624964C/T
4-1: 60666030T/A
4-1: 60747266C/T
4-1: 60814793T/A
4-1: 60843084T/C
4-1: 60924293C/T
4-1: 60924844C/A
4-1: 60924869C/A
4-1: 60926706G/A
4-1: 60982231C/T
4-1: 61201567G/A
4-1: 61247686C/G
4-1: 61322404T/C
4-1: 61358977C/G
4-1: 61416049C/T
4-1: 61431039T/C
4-1: 61454113A/G
4-1: 61567761A/T
4-1: 61609982A/G
4-1: 61657722G/A
4-1: 61705320A/C
4-1: 61869304C/T
4-1: 61925361A/T
4-1: 62123567C/A
4-1: 62195993G/A
4-1: 62290605C/T
4-1: 62484272T/A
4-1: 62548857T/G
4-1: 62634907G/A
4-1: 62660408C/A
4-1: 62778630C/T
4-1: 62890402T/A
4-1: 62898166T/C
4-1: 62942339G/A
4-1: 62984556T/C
4-1: 63087469A/T
4-1: 63099683T/C
4-1: 63112713A/G
4-1: 63193008C/T
4-1: 63286872C/G
4-1: 63316818T/A
4-1: 63335779G/C
4-1: 63368762T/C
4-1: 63551767C/T
4-1: 63682056T/C
4-1: 63785248C/T
4-1: 63879344C/T
4-1: 63892569G/A
4-1: 64063837T/C
4-1: 64116394T/C
4-1: 64148302T/A
4-1: 64217472A/G
4-1: 64226892C/T
4-1: 64243514T/C
4-1: 64326591T/C
4-1: 64338531A/C
4-1: 64353091C/A
4-1: 64388560T/C
4-1: 64464030T/C
4-1: 64494551T/C
4-1: 64557223A/G
4-1: 64558481A/G
4-1: 64616961T/A
4-1: 64793820T/G
4-1: 64885492G/T
4-1: 65046144C/T
4-1: 65121494T/G
4-1: 65171437C/T
4-1: 65209649G/T
4-1: 65253807G/A
4-1: 65297780C/T
4-1: 65321474C/A
4-1: 65396465C/T
4-1: 65463224T/C
4-1: 65542078T/A
4-1: 65597219G/C
4-1: 65609265A/G
4-1: 65754721T/C
4-1: 65833635G/A
4-1: 65912306C/T
4-1: 65997638T/C
4-1: 66136418C/A
4-1: 66341481G/A
4-1: 66362790C/T
4-1: 66375851T/C
4-1: 66411409T/G
4-1: 66430081C/T
4-1: 66473392T/A
4-1: 66597069C/T
4-1: 66709170G/C
4-1: 66803571A/G
4-1: 66865358T/A
4-1: 66888687A/G
4-1: 66991073C/T
4-1: 67015369T/C
4-1: 67050517G/A
4-1: 67124483C/T
4-1: 67197354C/T
4-1: 67198608A/G
4-1: 67284646C/G
4-1: 67284848G/A
4-1: 67284879G/A
4-1: 67284886T/G
4-1: 67289854C/T
4-1: 67345597C/T
4-1: 67373073A/T
4-1: 67400920A/T
4-1: 67438557C/T
4-1: 67535895G/C
4-1: 67571277G/T
4-1: 67674876C/T
4-1: 67676277C/T
4-1: 67815577G/A
4-1: 67867091A/G
4-1: 67956650C/T
4-1: 68151853C/T
4-1: 68173203A/G
4-1: 68268564A/G
4-1: 68306696T/A
4-1: 68310793C/T
4-1: 68310808C/T
4-1: 68310870G/A
4-1: 68310900C/A
4-1: 68311347G/A
4-1: 68411105G/A
4-1: 68592731G/A
4-1: 68623661A/C
4-1: 68856281T/A
4-1: 68939662A/G
4-1: 68941792C/G
4-1: 68941806G/A
4-1: 68941809A/G
4-1: 68941813C/T
4-1: 68941815T/C
4-1: 68941824G/T
4-1: 68941833G/T
4-1: 69144353A/C
4-1: 69234531T/C
4-1: 69308550T/C
4-1: 69440236A/G
4-1: 69603927G/A
4-1: 69618668C/T
4-1: 69746176G/T
4-1: 69767115G/A
4-1: 69777520G/A
4-1: 69814934A/G
4-1: 69967186T/C
4-1: 70098589T/C
4-1: 70188441G/A
4-1: 70235833C/A
4-1: 70252958G/A
4-1: 70273695T/C
4-1: 70336065A/G
4-1: 70406853C/T
4-1: 70417308T/C
4-1: 70482181G/A
4-1: 70578811A/G
4-1: 70615265C/T
4-1: 70703958G/T
4-1: 70937661C/T
4-1: 71034401G/A
4-1: 71090252C/A
4-1: 71188521C/G
4-1: 71211087G/T
4-1: 71264319A/G
4-1: 71264388G/A
4-1: 71266560C/T
4-1: 71268958G/C
4-1: 71268976T/A
4-1: 71295625G/A
4-1: 71387188T/A
4-1: 71437530G/A
4-1: 71558512C/T
4-1: 71600814A/G
4-1: 71646641T/G
4-1: 71770346T/G
4-1: 71854874G/A
4-1: 71900705C/T
4-1: 71912832C/T
4-1: 71976247G/A
4-1: 72047808C/T
4-1: 72140391T/C
4-1: 72150689T/C
4-1: 72201126T/A
4-1: 72218005C/T
4-1: 72324629T/G
4-1: 72467523C/T
4-1: 72646071C/T
4-1: 72713133G/A
4-1: 72830509T/G
4-1: 72985515T/C
4-1: 73041567G/A
4-1: 73167112G/A
4-1: 73249678G/C
4-1: 73413176A/C
4-1: 73505846C/T
4-1: 73554388C/T
4-1: 73634358G/A
4-1: 73639362T/C
4-1: 73690317T/C
4-1: 73801265T/C
4-1: 73886994T/C
4-1: 74108934T/C
4-1: 74237042T/C
4-1: 74288900A/T
4-1: 74372302G/T
4-1: 74455492C/T
4-1: 74539024T/C
4-1: 74671906T/A
4-1: 74683564C/G
4-1: 74823137C/T
4-1: 74836724A/T
4-1: 74955731A/G
4-1: 74963538G/C
4-1: 75038731C/A
4-1: 75173840A/G
4-1: 75181158G/A
4-1: 75200201C/T
4-1: 75273872G/C
4-1: 75287664A/G
4-1: 75341019T/G
4-1: 75366414A/G
4-1: 75438614T/C
4-1: 75449354C/T
4-1: 75465430T/C
4-1: 75483645T/C
4-1: 75495249A/G
4-1: 75520311C/T
4-1: 75533450C/G
4-1: 75558072A/G
4-1: 75570289T/C
4-1: 75589661G/T
4-1: 75636765G/A
4-1: 75647200T/C
4-1: 75667434T/C
4-1: 75681926C/T
4-1: 75693022G/A
4-1: 75726432A/T
4-1: 75737119T/C
4-1: 75737131T/C
4-1: 75765535C/T
4-1: 75803259C/T
4-1: 75809223T/A
4-1: 75846326C/A
4-1: 75889899G/A
4-1: 75942795C/T
4-1: 75953560T/C
4-1: 75979277A/G
4-1: 76015728T/C
4-1: 76062108A/C
4-1: 76100854A/T
4-1: 76171293C/T
4-1: 76217817A/G
4-1: 76255990T/C
4-1: 76283133A/G
4-1: 76320393T/C
4-1: 76327781C/A
4-1: 76338845C/G
4-1: 76415571T/C
4-1: 76449523G/T
4-1: 76467711C/A
4-1: 76478331T/C
4-1: 76520726C/T
4-1: 76571858A/T
4-1: 76583422A/G
4-1: 76607827C/A
4-1: 76710048A/G
4-1: 76737281A/C
4-1: 76771754C/T
4-1: 76849726A/G
4-1: 76869140C/T
4-1: 76945666C/T
4-1: 76999008C/T
4-1: 77116653T/C
4-1: 77124771A/G
4-1: 77149495A/G
4-1: 77178975A/G
4-1: 77240427T/C
4-1: 77254798A/T
4-1: 77268987T/C
4-1: 77288418A/C
4-1: 77301975G/A
4-1: 77314420T/C
4-1: 77330463C/A
4-1: 77337148A/C
4-1: 77350721A/C
4-1: 77370718A/G
4-1: 77415993G/A
4-1: 77441310G/C
4-1: 77471641T/A
4-1: 77506978C/T
4-1: 77535237G/C
4-1: 77571118T/A
4-1: 77611212T/C
4-1: 77626702A/C
4-1: 77673776G/A
4-1: 77737791G/T
4-1: 77749684G/T
4-1: 77770089C/T
4-1: 77803713C/T
4-1: 77805783T/C
4-1: 77831200A/T
4-1: 77916876C/T
4-1: 77954834G/A
4-1: 77980734A/G
4-1: 78007231A/T
4-1: 78042312C/A
4-1: 78108686G/C
4-1: 78147935T/C
4-1: 78163802T/C
4-1: 78164647G/A
4-1: 78164876A/G
4-1: 78193042G/A
4-1: 78246668T/A
4-1: 78317874T/G
4-1: 78358495G/T
4-1: 78379078T/C
4-1: 78407638C/A
4-1: 78422172C/G
4-1: 78452735T/A
4-1: 78467186G/C
4-1: 78495101G/T
4-1: 78550228G/A
4-1: 78568151G/C
4-1: 78596181G/A
4-1: 78621843C/A
4-1: 78655357A/G
4-1: 78688230C/T
4-1: 78739055G/T
4-1: 78764743G/A
4-1: 78858187A/T
4-1: 78900647A/T
4-1: 78936057C/T
4-1: 78963005A/T
4-1: 79013838A/G
4-1: 79059730T/C
4-1: 79157957C/T
4-1: 79191779A/T
4-1: 79204773T/C
4-1: 79221258T/C
4-1: 79286840C/T
4-1: 79407160A/G
4-1: 79415710G/T
4-1: 79442363G/A
4-1: 79501602T/C
4-1: 79521062A/C
4-1: 79539919G/T
4-1: 79550756T/C
4-1: 79567248A/G
4-1: 79611462A/G
4-1: 79622555C/T
4-1: 79654619T/G
4-1: 79690944A/T
4-1: 79879623C/T
4-1: 79880645C/T
4-1: 79891861T/A
4-1: 79941257A/G
4-1: 79977807C/A
4-1: 80026751G/A
4-1: 80070393G/A
4-1: 80111336A/T
4-1: 80248479C/T
4-1: 80283770G/T
4-1: 80320497G/C
4-1: 80381554G/A
4-1: 80409628A/G
4-1: 80478423C/T
4-1: 80534328C/T
4-1: 80579566T/C
4-1: 80603921T/A
4-1: 80610021A/T
4-1: 80615521T/A
4-1: 80620797T/G
4-1: 80623115A/G
4-1: 80624489G/A
4-1: 80720733T/C
4-1: 80735079A/C
4-1: 80822304G/A
4-1: 80912849A/C
4-1: 80952278G/A
4-1: 80994082T/C
4-1: 81057570T/C
4-1: 81109375C/T
4-1: 81211573C/A
4-1: 81311304A/T
4-1: 81375554A/T
4-1: 81419057A/G
4-1: 81467174T/G
4-1: 81752708A/G
4-1: 81881799C/T
4-1: 81961551C/T
4-1: 82018103A/C
4-1: 82094061A/T
4-1: 82151376C/T
4-1: 82161410G/A
4-1: 82189264A/T
4-1: 82240946A/T
4-1: 82289264G/T
4-1: 82323183A/T
4-1: 82517866G/A
4-1: 82667786T/A
4-1: 82894154A/G
4-1: 82987226T/C
4-1: 83070913T/C
4-1: 83133335C/A
4-1: 83232014A/T
4-1: 83265289G/A
4-1: 83340051G/T
4-1: 83341893C/T
4-1: 83341926T/A
4-1: 83342868A/G
4-1: 83343051G/A
4-1: 83343386A/G
4-1: 83343556C/T
4-1: 83343842A/G
4-1: 83343915G/C
4-1: 83395036T/A
4-1: 83491177T/C
4-1: 83524786G/A
4-1: 83549807A/G
4-1: 83649032G/T
4-1: 83727034G/A
4-1: 83890784T/C
4-1: 83932002C/T
4-1: 83962434A/T
4-1: 84005794T/G
4-1: 84025453T/G
4-1: 84282607G/A
4-1: 84314532T/A
4-1: 84467448A/T
4-1: 84479729A/G
4-1: 84519751G/A
4-1: 84603555A/T
4-1: 84657282C/T
4-1: 84834229A/T
4-1: 84871494T/C
4-1: 84875085A/G
4-1: 84875281G/T
4-1: 84875902C/G
4-1: 84884222A/G
4-1: 84906003C/A
4-1: 84960683A/G
4-1: 85050627G/A
4-1: 85174016G/A
4-1: 85226256G/T
4-1: 85422092T/C
4-1: 85457341G/A
4-1: 85520407T/G
4-1: 85587647G/A
4-1: 85619893A/G
4-1: 85764834T/A
4-1: 85876810C/A
4-1: 86073936T/G
4-1: 86150005G/A
4-1: 86184039G/C
4-1: 86267313A/G
4-1: 86296405A/T
4-1: 86527010T/C
4-1: 86738501A/G
4-1: 86799786C/T
4-1: 86817585T/A
4-1: 86887363A/T
4-1: 86985339T/C
4-1: 87102068G/A
4-1: 87576581C/A
4-1: 87640931C/T
4-1: 87706819C/A
4-1: 87797496A/G
4-2: 584737G/C
4-2: 716781A/C
4-2: 1329711A/G
4-2: 1416937G/A
4-2: 1442780G/A
4-2: 1656124A/G
4-2: 1676209C/G
4-2: 1733643T/C
4-2: 1807423C/A
4-2: 1912230T/A
4-2: 2035319T/A
4-2: 2241933C/T
4-2: 2415073G/T
4-2: 2500957G/A
4-2: 2514674T/C
4-2: 2569028A/G
4-2: 2718769C/T
4-2: 2721174T/A
4-2: 2773606G/T
4-2: 3018127T/C
4-2: 3040745G/A
4-2: 3054869T/C
4-2: 3070005G/A
4-2: 3081511C/T
4-2: 3158972C/T
4-2: 3165516T/C
4-2: 3234218A/G
4-2: 3378494A/G
4-2: 3414729T/C
4-2: 3462911A/G
4-2: 3541824A/T
4-2: 3779509A/T
4-2: 3879969C/G
4-2: 3906170C/T
4-2: 4120649T/C
4-2: 4179714A/T
4-2: 4274516G/T
4-2: 4369657C/T
4-2: 4522785C/T
4-2: 4610764G/A
4-2: 4678867T/C
4-2: 4685969A/G
4-2: 4709166G/A
4-2: 4732955G/A
4-2: 4733609T/C
4-2: 4815126C/T
4-2: 4959878C/G
4-2: 4963515C/T
4-2: 5126854G/A
4-2: 5144054T/C
4-2: 5171745C/A
4-2: 5173083T/C
4-2: 5304397T/A
4-2: 5308558A/G
4-2: 5309034G/C
4-2: 5309273A/T
4-2: 5309741A/T
4-2: 5319104A/T
4-2: 5334174T/G
4-2: 5393616G/A
4-2: 5528237T/C
4-2: 5685517G/A
4-2: 5724110G/T
4-2: 5784718T/A
4-2: 5821476A/G
4-2: 5861047T/C
4-2: 5897148C/T
4-2: 6146679T/C
4-2: 6155440T/A
4-2: 6172668C/T
4-2: 6240382G/C
4-2: 6279844C/T
4-2: 6379081T/G
4-2: 6390728T/C
4-2: 6488488G/A
4-2: 6561834G/A
4-2: 6615945G/T
4-2: 6773194T/G
4-2: 6811552C/T
4-2: 6840942T/G
4-2: 6868591T/G
4-2: 6896301G/A
4-2: 6898034A/G
4-2: 6909876T/C
4-2: 6954541T/C
4-2: 6988407G/A
4-2: 7001882G/T
4-2: 7081490G/A
4-2: 7114392T/A
4-2: 7139574C/T
4-2: 7267349G/A
4-2: 7375472G/T
4-2: 7425104T/C
4-2: 7491096G/T
4-2: 7598650T/A
4-2: 7627017A/C
4-2: 7665360A/G
4-2: 7744942G/A
4-2: 7867319A/G
4-2: 7992783T/C
4-2: 8026891T/A
4-2: 8093253C/T
4-2: 8103753A/G
4-2: 8108660T/C
4-2: 8110364T/C
4-2: 8110861C/T
4-2: 8114911A/G
4-2: 8138139C/T
4-2: 8149313T/G
4-2: 8329044A/G
4-2: 8431530G/T
4-2: 8503323G/C
4-2: 8505648G/A
4-2: 8505713G/A
4-2: 8506339G/A
4-2: 8506461C/G
4-2: 8506672A/G
4-2: 8506726G/A
4-2: 8506786G/A
4-2: 8507358C/T
4-2: 8507703C/T
4-2: 8507884G/A
4-2: 8507988G/A
4-2: 8508024T/C
4-2: 8508094C/G
4-2: 8508294T/C
4-2: 8508336T/G
4-2: 8509613G/A
4-2: 8509875G/A
4-2: 8518913G/A
4-2: 8529799C/T
4-2: 8555246A/C
4-2: 8605706C/A
4-2: 8637507A/G
4-2: 8688462A/T
4-2: 8742753A/G
4-2: 8789326A/G
4-2: 8856916A/G
4-2: 8895637G/T
4-2: 8895640T/G
4-2: 8920350C/T
4-2: 9053931G/A
4-2: 9060950A/T
4-2: 9089399G/A
4-2: 9102271C/T
4-2: 9116790A/T
4-2: 9133788A/T
4-2: 9168938A/T
4-2: 9179160C/A
4-2: 9203673A/G
4-2: 9215016T/C
4-2: 9228798A/C
4-2: 9238557T/C
4-2: 9239307A/G
4-2: 9249637T/C
4-2: 9249955T/C
4-2: 9264477G/T
4-2: 9301118G/C
4-2: 9315889G/A
4-2: 9316184T/G
4-2: 9329410G/A
4-2: 9346739C/A
4-2: 9367397G/C
4-2: 9377769T/C
4-2: 9417848C/T
4-2: 9418741A/T
4-2: 9419492C/A
4-2: 9419784G/T
4-2: 9420354G/A
4-2: 9422409C/T
4-2: 9422560A/T
4-2: 9422636G/A
4-2: 9429777G/A
4-2: 9445449T/A
4-2: 9458129G/A
4-2: 9620388C/T
4-2: 9789657T/C
4-2: 9834869T/A
4-2: 9892866G/A
4-2: 9976382G/A
4-2: 9985755C/T
4-2: 10021320T/C
4-2: 10054508C/A
4-2: 10080052C/A
4-2: 10130332G/A
4-2: 10170262C/T
4-2: 10187501C/T
4-2: 10212072C/T
4-2: 10213839T/C
4-2: 10271873T/C
4-2: 10293120C/T
4-2: 10333430C/G
4-2: 10339100C/G
4-2: 10368960T/C
4-2: 10391758G/C
4-2: 10435576C/T
4-2: 10453565A/T
4-2: 10471035G/A
4-2: 10491793A/T
4-2: 10506817C/A
4-2: 10589671G/A
4-2: 10602006T/A
4-2: 10652649A/G
4-2: 10663471G/A
4-2: 10835412G/C
4-2: 10850725C/T
4-2: 10898563C/G
4-2: 10996301T/G
4-2: 11063098G/C
4-2: 11123398G/A
4-2: 11160273T/C
4-2: 11235405G/A
4-2: 11275059T/C
4-2: 11291333G/A
4-2: 11338984C/T
4-2: 11443060C/T
4-2: 11575065C/A
4-2: 11592447G/A
4-2: 11623602G/A
4-2: 11712760G/T
4-2: 11731435T/A
4-2: 11787242T/A
4-2: 11823570C/T
4-2: 11860527C/A
4-2: 11860791G/T
4-2: 11898594G/A
4-2: 11927338G/T
4-2: 12058854A/G
4-2: 12060234C/G
4-2: 12227468C/T
4-2: 12255409G/A
4-2: 12311382G/A
4-2: 12356572G/A
4-2: 12376553A/T
4-2: 12390442C/A
4-2: 12431728G/A
4-2: 12486523G/A
4-2: 12527496C/T
4-2: 12599955T/G
4-2: 12617471T/G
4-2: 12682439C/T
4-2: 12713231G/A
4-2: 12735109G/C
4-2: 12771712G/A
4-2: 12780640T/G
4-2: 12812961A/T
4-2: 12879069T/A
4-2: 12918789G/A
4-2: 12970381A/T
4-2: 12988329T/C
4-2: 13008094C/T
4-2: 13084902T/C
4-2: 13100853T/C
4-2: 13172786A/T
4-2: 13173383G/A
4-2: 13173402G/A
4-2: 13173491C/T
4-2: 13220522A/G
4-2: 13224395T/C
4-2: 13227678A/T
4-2: 13227787A/T
4-2: 13235430C/G
4-2: 13235553C/A
4-2: 13235708C/T
4-2: 13235810T/G
4-2: 13236314T/C
4-2: 13236902T/G
4-2: 13237004C/T
4-2: 13237125A/G
4-2: 13237171G/A
4-2: 13237238G/C
4-2: 13237324C/T
4-2: 13237377T/C
4-2: 13237479C/T
4-2: 13237599G/A
4-2: 13237704G/A
4-2: 13237805C/T
4-2: 13237851T/C
4-2: 13237907G/A
4-2: 13238206G/A
4-2: 13238217C/T
4-2: 13264102G/A
4-2: 13289196T/C
4-2: 13399927T/A
4-2: 13551009T/C
4-2: 13566951C/T
4-2: 13581221C/T
4-2: 13604714A/G
4-2: 13605023C/G
4-2: 13605156A/G
4-2: 13605323G/A
4-2: 13605420G/A
4-2: 13605497A/C
4-2: 13616497T/C
4-2: 13696021C/T
4-2: 13822763G/T
4-2: 13840922C/T
4-2: 13852059C/T
4-2: 13879363G/A
4-2: 13882437A/G
4-2: 13892522A/G
4-2: 13939084C/G
4-2: 13957936A/T
4-2: 14123703G/A
4-2: 14132825G/A
4-2: 14136851T/C
4-2: 14182346T/A
4-2: 14227901A/G
4-2: 14237072T/C
4-2: 14346283C/T
4-2: 14391464G/C
4-2: 14391633C/T
4-2: 14424440C/T
4-2: 14496570T/G
4-2: 14537309A/G
4-2: 14586463C/T
4-2: 14618274C/T
4-2: 14656708T/A
4-2: 14727849G/A
4-2: 14734631A/C
4-2: 14734755G/A
4-2: 14734868G/T
4-2: 14757262T/A
4-2: 14799411G/T
4-2: 14826132G/A
4-2: 14849281C/T
4-2: 14936570T/C
4-2: 14942016A/C
4-2: 14963201T/A
4-2: 14964443C/A
4-2: 14964455T/A
4-2: 15121385T/C
4-2: 15131439G/A
4-2: 15339831G/A
4-2: 15359896T/G
4-2: 15371431T/G
4-2: 15478754T/C
4-2: 15513799T/C
4-2: 15538862G/A
4-2: 15665359G/C
4-2: 15755550G/T
4-2: 15789700T/C
4-2: 15799797C/T
4-2: 15872859C/T
4-2: 15934464A/C
4-2: 15977516C/G
4-2: 16004280A/T
4-2: 16069899C/T
4-2: 16127862G/T
4-2: 16143641G/A
4-2: 16171474A/G
4-2: 16221957T/A
4-2: 16261598A/G
4-2: 16277857G/A
4-2: 16371564T/A
4-2: 16402931G/T
4-2: 16421220A/G
4-2: 16433580T/C
4-2: 16445704T/C
4-2: 16519591C/A
4-2: 16551498G/T
4-2: 16630651A/G
4-2: 16674914G/C
4-2: 16676616A/G
4-2: 16706241A/G
4-2: 16788805A/G
4-2: 16846113T/C
4-2: 16924536G/T
4-2: 16948104G/A
4-2: 16949813C/T
4-2: 16950350G/T
4-2: 17035237C/A
4-2: 17058556T/C
4-2: 17062766C/T
4-2: 17102944T/G
4-2: 17103330G/A
4-2: 17103576G/A
4-2: 17106910A/G
4-2: 17123768G/A
4-2: 17148300G/T
4-2: 17192375C/T
4-2: 17195025C/A
4-2: 17197251A/C
4-2: 17197774G/A
4-2: 17198589T/C
4-2: 17202940G/A
4-2: 17213127A/G
4-2: 17213143T/A
4-2: 17213597T/A
4-2: 17213737G/A
4-2: 17268494T/C
4-2: 17278842T/A
4-2: 17281648A/T
4-2: 17338677G/A
4-2: 17363746A/C
4-2: 17375487A/G
4-2: 17378004T/C
4-2: 17397076A/G
4-2: 17397438T/C
4-2: 17406858C/A
4-2: 17415018A/C
4-2: 17496331G/A
4-2: 17595167T/G
4-2: 17606776A/G
4-2: 17650207C/T
4-2: 17664682G/A
4-2: 17689823T/C
4-2: 17694933T/C
4-2: 17698477G/A
4-2: 17698729G/A
4-2: 17700834A/C
4-2: 17721604C/T
4-2: 17743717C/T
4-2: 17812633T/G
4-2: 17832561T/A
4-2: 17853134T/A
4-2: 17921831A/G
4-2: 17966855G/T
4-2: 18005084T/A
4-2: 18046255C/G
4-2: 18076501G/A
4-2: 18141386T/A
4-2: 18239521G/A
4-2: 18240198G/C
4-2: 18261638T/A
4-2: 18282520T/A
4-2: 18299033T/C
4-2: 18380186A/G
4-2: 18390866A/G
4-2: 18627825G/C
4-2: 18632027G/A
4-2: 18940509T/A
4-2: 18951871A/T
4-2: 19018527G/A
4-2: 19045457G/A
4-2: 19078187G/A
4-2: 19089312C/T
4-2: 19089738G/A
4-2: 19132794T/C
4-2: 19265410T/C
4-2: 19278393G/A
4-2: 19290490G/C
4-2: 19313828C/T
4-2: 19325272A/T
4-2: 19338964A/T
4-2: 19341080A/G
4-2: 19353485T/C
4-2: 19364166T/A
4-2: 19425001G/A
4-2: 19500813A/T
4-2: 19535341C/A
4-2: 19612321C/G
4-2: 19627699G/T
4-2: 19659638G/A
4-2: 19704709C/G
4-2: 19802957G/A
4-2: 19811341C/G
4-2: 19841405A/T
4-2: 19926240A/G
4-2: 19975916C/A
4-2: 19978388A/G
4-2: 19998086A/G
4-2: 20020389A/T
4-2: 20047450A/T
4-2: 20073656T/C
4-2: 20166798T/G
4-2: 20174050C/G
4-2: 20197419G/A
4-2: 20213824C/T
4-2: 20372992C/T
4-2: 20374541T/A
4-2: 20451932C/A
4-2: 20512116A/G
4-2: 20546521G/T
4-2: 20579812G/T
4-2: 20640133C/T
4-2: 20654149G/A
4-2: 20664945C/T
4-2: 20682168A/G
4-2: 20735179C/T
4-2: 20852200A/G
4-2: 20923382C/A
4-2: 20933577A/T
4-2: 20933585C/T
4-2: 20933587C/T
4-2: 20934216C/T
4-2: 20992323T/A
4-2: 21013123C/T
4-2: 21017365A/T
4-2: 21043651A/G
4-2: 21137769A/C
4-2: 21155963C/T
4-2: 21166019A/G
4-2: 21189125T/G
4-2: 21230680T/C
4-2: 21266636G/C
4-2: 21334225T/A
4-2: 21334670G/A
4-2: 21352140G/A
4-2: 21485027T/C
4-2: 21534989G/A
4-2: 21545686A/G
4-2: 21575239T/A
4-2: 21592769T/C
4-2: 21678429C/T
4-2: 21739423G/C
4-2: 21766840A/C
4-2: 21856033G/T
4-2: 21906045G/A
4-2: 22003696A/G
4-2: 22025506A/G
4-2: 22057725A/T
4-2: 22100233T/C
4-2: 22118656G/C
4-2: 22118972G/T
4-2: 22130547G/A
4-2: 22206733T/A
4-2: 22281854G/A
4-2: 22299447G/C
4-2: 22314265G/A
4-2: 22334467C/T
4-2: 22361913T/C
4-2: 22421751G/T
4-2: 22472024A/G
4-2: 22536855A/G
4-2: 22537783A/T
4-2: 22541076T/C
4-2: 22617330C/G
4-2: 22662667G/C
4-2: 22760314G/A
4-2: 22771996G/A
4-2: 22785017C/T
4-2: 22790290A/G
4-2: 22790632G/T
4-2: 22790650C/T
4-2: 22795188A/C
4-2: 22818569A/G
4-2: 22831683T/A
4-2: 22905630C/G
4-2: 22905643A/G
4-2: 22905752C/T
4-2: 22905963C/G
4-2: 22907812A/G
4-2: 22910208A/G
4-2: 22920323T/C
4-2: 22920800C/T
4-2: 22969676T/C
4-2: 23018140T/C
4-2: 23062940G/A
4-2: 23091009C/A
4-2: 23170069G/A
4-2: 23180701T/C
4-2: 23181355A/G
4-2: 23181670C/A
4-2: 23203932G/A
4-2: 23212586A/C
4-2: 23220861G/A
4-2: 23286183A/T
4-2: 23304270C/T
4-2: 23364217T/A
4-2: 23399296A/G
4-2: 23410271T/C
4-2: 23482973G/A
4-2: 23518273T/G
4-2: 23543307C/A
4-2: 23774669A/C
4-2: 23864051G/C
4-2: 23896453C/T
4-2: 23908341T/C
4-2: 24002130C/T
4-2: 24003708T/A
4-2: 24014998C/A
4-2: 24017092G/A
4-2: 24018792T/A
4-2: 24020559A/C
4-2: 24074585C/A
4-2: 24132187C/T
4-2: 24201678A/G
4-2: 24268800C/T
4-2: 24353024T/C
4-2: 24444261A/G
4-2: 24487672A/T
4-2: 24488376A/G
4-2: 24488499G/T
4-2: 24488621T/C
4-2: 24488723A/G
4-2: 24489009A/C
4-2: 24489201T/A
4-2: 24489337T/C
4-2: 24490559T/C
4-2: 24490741G/A
4-2: 24491070T/C
4-2: 24491288G/A
4-2: 24491505T/C
4-2: 24491557T/C
4-2: 24491751C/A
4-2: 24491864A/G
4-2: 24492027G/A
4-2: 24492296C/A
4-2: 24492412T/G
4-2: 24492573C/T
4-2: 24495889G/A
4-2: 24497448G/A
4-2: 24497495C/T
4-2: 24498579T/A
4-2: 24500817A/G
4-2: 24501039G/A
4-2: 24501495G/A
4-2: 24501680G/T
4-2: 24502227T/C
4-2: 24502399G/A
4-2: 24502579C/T
4-2: 24502694C/T
4-2: 24502714C/T
4-2: 24502812G/C
4-2: 24502913A/T
4-2: 24503067C/G
4-2: 24503097A/G
4-2: 24503181T/A
4-2: 24503216T/A
4-2: 24503291C/A
4-2: 24503440T/A
4-2: 24503577A/G
4-2: 24503715G/A
4-2: 24503717A/T
4-2: 24503771A/T
4-2: 24504118G/A
4-2: 24506336G/A
4-2: 24508780T/A
4-2: 24602943A/T
4-2: 24679169C/T
4-2: 24680837A/G
4-2: 24691647C/T
4-2: 24745841C/T
4-2: 24870640G/T
4-2: 24935943C/G
4-2: 24938130A/C
4-2: 24951584C/T
4-2: 24951927A/C
4-2: 24951929T/C
4-2: 24951986T/G
4-2: 25042484C/T
4-2: 25141343T/C
4-2: 25148695G/A
4-2: 25165336G/A
4-2: 25186995T/C
4-2: 25339460A/T
4-2: 25367083A/T
4-2: 25460511T/C
4-2: 25487936G/T
4-2: 25509007C/G
4-2: 25540728T/A
4-2: 25609949G/T
4-2: 25680885A/C
4-2: 25706609C/G
4-2: 25711157G/A
4-2: 25744287T/G
4-2: 25772160G/A
4-2: 25772185T/C
4-2: 25808662A/G
4-2: 25974718A/T
4-2: 25988473C/A
4-2: 26008218C/A
4-2: 26044658G/A
4-2: 26129592T/C
4-2: 26180222G/A
4-2: 26277532G/C
4-2: 26329743A/G
4-2: 26353936C/A
4-2: 26437888T/A
4-2: 26485679A/G
4-2: 26517602T/C
4-2: 26544955G/A
4-2: 26588016T/C
4-2: 26634739A/G
4-2: 26669282G/T
4-2: 26681430G/A
4-2: 26694077G/C
4-2: 26736609G/A
4-2: 26779319G/A
4-2: 26902501A/G
4-2: 27059557A/G
4-2: 27068495G/A
4-2: 27122463C/A
4-2: 27148299A/T
4-2: 27166146T/C
4-2: 27276857G/A
4-2: 27409007A/T
4-2: 27416270C/T
4-2: 27416290A/T
4-2: 27416371G/A
4-2: 27425967T/G
4-2: 27427410T/C
4-2: 27453386C/G
4-2: 27503079C/T
4-2: 27598069A/T
4-2: 27627205G/T
4-2: 27652330C/G
4-2: 27681792T/C
4-2: 27708303A/T
4-2: 27782806G/A
4-2: 27884779A/T
4-2: 27888595T/C
4-2: 27912218T/C
4-2: 27927099T/C
4-2: 27962219C/T
4-2: 28057295A/G
4-2: 28111888G/A
4-2: 28143579C/T
4-2: 28172848C/T
4-2: 28186772T/A
4-2: 28233634G/A
4-2: 28243065C/T
4-2: 28243785T/C
4-2: 28257448A/G
4-2: 28280976T/G
4-2: 28373299T/C
4-2: 28406994C/A
4-2: 28482337C/T
4-2: 28484936T/C
4-2: 28484960T/C
4-2: 28484969C/T
4-2: 28484984T/C
4-2: 28485006T/C
4-2: 28485374T/G
4-2: 28485420A/G
4-2: 28485461C/T
4-2: 28652321T/A
4-2: 28688515T/C
4-2: 28727747T/A
4-2: 28761127A/G
4-2: 28761302T/C
4-2: 28762041A/G
4-2: 28762231T/A
4-2: 28779502T/A
4-2: 28799311C/T
4-2: 28847629C/T
4-2: 28881367C/T
4-2: 28913153G/T
4-2: 28964907G/A
4-2: 28975850A/C
4-2: 29010698G/A
4-2: 29034428G/A
4-2: 29062576T/A
4-2: 29144079A/C
4-2: 29176731C/A
4-2: 29220552G/A
4-2: 29305872A/C
4-2: 29325750G/A
4-2: 29440716C/T
4-2: 29660148G/A
4-2: 29717518C/T
4-2: 29786999T/C
4-2: 29883425A/T
4-2: 29932087G/T
4-2: 29987382C/A
4-2: 29992927G/T
4-2: 30002681A/T
4-2: 30017268C/G
4-2: 30028955T/A
4-2: 30054945C/A
4-2: 30079945A/G
4-2: 30091760A/G
4-2: 30103150A/T
4-2: 30103153C/G
4-2: 30118052C/T
4-2: 30154270A/G
4-2: 30160296G/A
4-2: 30173903C/A
4-2: 30210213C/T
4-2: 30226991A/G
4-2: 30287565C/A
4-2: 30363732T/A
4-2: 30417188C/A
4-2: 30442481C/A
4-2: 30520498T/A
4-2: 30521587G/A
4-2: 30521619G/A
4-2: 30523511C/T
4-2: 30533846T/A
4-2: 30596989T/A
4-2: 30614054A/C
4-2: 30633130G/T
4-2: 30661017A/G
4-2: 30679739G/A
4-2: 30851286G/A
4-2: 30861505T/C
4-2: 30866892C/T
4-2: 30867064A/G
4-2: 30884304G/A
4-2: 30925219T/C
4-2: 30948787A/G
4-2: 30965719C/A
4-2: 31008089A/T
4-2: 31041906C/T
4-2: 31080339A/G
4-2: 31223271C/T
4-2: 31484475C/T
4-2: 31521880G/A
4-2: 31670847G/A
4-2: 31714115T/G
4-2: 31840787G/A
4-2: 31875821T/G
4-2: 31919487T/C
4-2: 31949494A/C
4-2: 32031955A/T
4-2: 32037367T/C
4-2: 32037385A/C
4-2: 32038533A/T
4-2: 32038639G/A
4-2: 32043719T/G
4-2: 32071376G/A
4-2: 32104696T/G
4-2: 32273656A/T
4-2: 32276257C/T
4-2: 32361172C/T
4-2: 32428482T/C
4-2: 32431737G/A
4-2: 32434392A/C
4-2: 32435552C/A
4-2: 32452816A/G
4-2: 32525133T/A
4-2: 32629216A/C
4-2: 32665978C/T
4-2: 32675812A/C
4-2: 32692883A/C
4-2: 32777995T/C
4-2: 32817337G/A
4-2: 32865847T/C
4-2: 33008277T/A
4-2: 33021935C/T
4-2: 33113564C/T
4-2: 33162764C/T
4-2: 33211551G/A
4-2: 33291649T/A
4-2: 33365201A/G
4-2: 33523972T/G
4-2: 33526507A/G
4-2: 33618683A/G
4-2: 33696909A/G
4-2: 33748452A/G
4-2: 33809047G/C
4-2: 33823876A/G
4-2: 34023137G/A
4-2: 34265820G/A
4-2: 34507590A/C
4-2: 34815573C/A
4-2: 34821777A/G
4-2: 34821885A/T
4-2: 34821971C/A
4-2: 34822372C/G
4-2: 34822729A/T
4-2: 34824350G/C
4-2: 34829658A/C
4-2: 35069882G/A
4-2: 35071136G/A
4-2: 35167004G/A
4-2: 35207348G/A
4-2: 35253881G/A
4-2: 35281017G/A
4-2: 35293461C/T
4-2: 35330539C/T
4-2: 35343298T/C
4-2: 35348881C/G
4-2: 35421257T/G
4-2: 35424451C/T
4-2: 35424655A/G
4-2: 35424810T/C
4-2: 35430171A/T
4-2: 35450733G/A
4-2: 35466283T/C
4-2: 35467809G/C
4-2: 35484214T/C
4-2: 35492252T/G
4-2: 35574828T/C
4-2: 35608217C/T
4-2: 35631482C/T
4-2: 35643083C/A
4-2: 35693743G/A
4-2: 35707985A/G
4-2: 35734673T/C
4-2: 35779417A/C
4-2: 35779473A/G
4-2: 35781004A/G
4-2: 35781201G/A
4-2: 35781527G/A
4-2: 35783499G/T
4-2: 35809927T/G
4-2: 35841114C/T
4-2: 35871129A/G
4-2: 35921235A/G
4-2: 35959871T/C
4-2: 36021361A/T
4-2: 36129658C/T
4-2: 36160604A/G
4-2: 36181428A/C
4-2: 36383716G/A
4-2: 36431652A/C
4-2: 36492590C/A
4-2: 36495976G/T
4-2: 36496078T/A
4-2: 36496193T/G
4-2: 36503851A/C
4-2: 36536182G/T
4-2: 36577039A/C
4-2: 36607543G/A
4-2: 36661856T/G
4-2: 36733166C/T
4-2: 36799344G/A
4-2: 36801397C/G
4-2: 36813131A/G
4-2: 36897492C/T
4-2: 37008710T/C
4-2: 37019648G/C
4-2: 37020465C/T
4-2: 37036004A/G
4-2: 37124498G/T
4-2: 37158452C/A
4-2: 37243465T/A
4-2: 37324105T/G
4-2: 37324355G/T
4-2: 37325562A/G
4-2: 37330500A/T
4-2: 37340954G/A
4-2: 37437434G/C
4-2: 37541149A/T
4-2: 37627741G/A
4-2: 37753656C/T
4-2: 37798816C/G
4-2: 37919119A/C
4-2: 37940886T/C
4-2: 38091198A/G
4-2: 38160079A/G
4-2: 38293846T/A
4-2: 38320133T/C
4-2: 38390043G/T
4-2: 38520321T/A
4-2: 38569339G/A
4-2: 38595314C/T
4-2: 38629387G/A
4-2: 38659236A/C
4-2: 38743582T/A
4-2: 38783642C/T
4-2: 38810718C/T
4-2: 38952979T/C
4-2: 38969081G/A
4-2: 38984142T/C
4-2: 39096780A/G
4-2: 39150648C/A
4-2: 39185956A/G
4-2: 39294693C/T
4-2: 39304133A/T
4-2: 39334774C/T
4-2: 39368740C/G
4-2: 39420682T/G
4-2: 39434257T/C
4-2: 39550808G/A
4-2: 39558209G/A
4-2: 39630506T/C
4-2: 39677702G/A
4-2: 39718904C/T
4-2: 39752146G/A
4-2: 39820799T/A
4-2: 39828343A/.
4-2: 39857049G/C
4-2: 39857067A/G
4-2: 39857076G/T
4-2: 39857085C/A
4-2: 39857089A/C
4-2: 39857097C/T
4-2: 39857114C/T
4-2: 39857115C/A
4-2: 39857124A/G
4-2: 39857130T/A
4-2: 39857169T/C
4-2: 39857171T/C
4-2: 39860022G/A
4-2: 40034079G/T
4-2: 40061693A/C
4-2: 40096245C/T
4-2: 40143876A/G
4-2: 40181011A/T
4-2: 40182256C/T
4-2: 40310209C/T
4-2: 40362440G/C
4-2: 40542889T/C
4-2: 40626377A/C
4-2: 40690587G/A
4-2: 40744956G/A
4-2: 40783717C/T
4-2: 40832817T/C
4-2: 41005996A/G
4-2: 41020287C/T
4-2: 41033560A/G
4-2: 41088898G/A
4-2: 41094825C/G
4-2: 41156524C/A
4-2: 41202332C/T
4-2: 41215953A/T
4-2: 41217266C/A
4-2: 41243732T/A
4-2: 41245455G/T
4-2: 41247637A/T
4-2: 41334987C/G
4-2: 41448914G/A
4-2: 41463689C/A
4-2: 41465559T/C
4-2: 41476619G/A
4-2: 41574121G/A
4-2: 41621227A/C
4-2: 41632147T/C
4-2: 41704015G/A
4-2: 41801909G/A
4-2: 41919591G/T
4-2: 41931230G/T
4-2: 42011028T/C
4-2: 42038359C/A
4-2: 42092370G/T
4-2: 42098771G/A
4-2: 42124144A/G
4-2: 42194786G/T
4-2: 42259743A/G
4-2: 42402304T/C
4-2: 42413936G/A
4-2: 42428941C/T
4-2: 42567392A/T
4-2: 42578272C/T
4-2: 42673626G/A
4-2: 42770023T/G
4-2: 42772574G/C
4-2: 42791403C/T
4-2: 42815024A/C
4-2: 42879553A/C
4-2: 42897580A/G
4-2: 42908463T/C
4-2: 42914991T/A
4-2: 42918983A/G
4-2: 42945067C/A
4-2: 42957750C/T
4-2: 43008334A/T
4-2: 43063680C/A
4-2: 43078890T/C
4-2: 43092111T/A
4-2: 43107616G/C
4-2: 43281549A/T
4-2: 43347650A/T
4-2: 43372225A/G
4-2: 43385872C/G
4-2: 43513615C/T
4-2: 43609800G/T
4-2: 43662099C/T
4-2: 43676010G/A
4-2: 43720842C/G
4-2: 43757175A/G
4-2: 43771778A/C
4-2: 43784566G/A
4-2: 43879750A/G
4-2: 43906355T/C
4-2: 43916556G/C
4-2: 43927069T/G
4-2: 43971306C/A
4-2: 43999016A/G
4-2: 44010266T/C
4-2: 44013154C/A
4-2: 44034126G/A
4-2: 44081532C/T
4-2: 44129702A/C
4-2: 44144009G/A
4-2: 44170636G/A
4-2: 44205369C/T
4-2: 44251449T/C
4-2: 44309734A/T
4-2: 44310120T/C
4-2: 44310121T/C
4-2: 44327596G/C
4-2: 44367975G/A
4-2: 44434059A/T
4-2: 44482894T/C
4-2: 44509207C/T
4-2: 44591426C/T
4-2: 44591573C/T
4-2: 44619712C/T
4-2: 44672985T/G
4-2: 44686622T/C
4-2: 44705373C/T
4-2: 44739088C/T
4-2: 44760611C/A
4-2: 44767779T/A
4-2: 44771587A/T
4-2: 44771751G/A
4-2: 44774614T/G
4-2: 44774980C/T
4-2: 44775031T/C
4-2: 44775047G/T
4-2: 44776773T/C
4-2: 44798819T/C
4-2: 44807592T/C
4-2: 44809785A/G
4-2: 44809902C/T
4-2: 44812152A/G
4-2: 44823366T/C
4-2: 44823562T/A
4-2: 44824021C/G
4-2: 44829931G/A
4-2: 44874269G/A
4-2: 44882320A/G
4-2: 44903036A/T
4-2: 45011544G/A
4-2: 45108858T/C
4-2: 45141176C/A
4-2: 45193515C/T
4-2: 45241275A/G
4-2: 45302360A/T
4-2: 45319947A/G
4-2: 45413959G/T
4-2: 45469288A/G
4-2: 45469334C/T
4-2: 45469592G/A
4-2: 45469622G/A
4-2: 45469841C/T
4-2: 45469880G/A
4-2: 45470352T/A
4-2: 45470365G/A
4-2: 45470891A/G
4-2: 45470980A/T
4-2: 45518156G/C
4-2: 45646093A/G
4-2: 45744338C/T
4-2: 45825695G/A
4-2: 45848122G/T
4-2: 45884098T/C
4-2: 45983299T/G
4-2: 46020510T/G
4-2: 46062989A/G
4-2: 46090087C/A
4-2: 46110914T/C
4-2: 46127440A/C
4-2: 46186463G/A
4-2: 46226069A/T
4-2: 46279823T/G
4-2: 46430759G/A
4-2: 46435793A/C
4-2: 46490847T/C
4-2: 46660916C/T
4-2: 46683053G/A
4-2: 46708173C/T
4-2: 46781707G/A
4-2: 46854065A/G
4-2: 47013040A/T
4-2: 47048419A/G
4-2: 47085225C/T
4-2: 47160056T/G
4-2: 47234312A/C
4-2: 47235615T/C
4-2: 47285784G/A
4-2: 47332556G/A
4-2: 47358803A/T
4-2: 47465637A/G
4-2: 47543024G/A
4-2: 47582602A/G
4-2: 47639150G/C
4-2: 47650736T/C
4-2: 47663951C/T
4-2: 47687165A/G
4-2: 47721863G/A
4-2: 47754550T/C
4-2: 47807111G/T
4-2: 47842460G/A
4-2: 47883593C/G
4-2: 47923057T/G
4-2: 47958119G/A
4-2: 47999361G/T
4-2: 48001290G/T
4-2: 48034781G/A
4-2: 48133272G/A
4-2: 48216074A/G
4-2: 48261341A/T
4-2: 48261698C/T
4-2: 48336824A/T
4-2: 48347542A/G
4-2: 48357921T/G
4-2: 48358075A/C
4-2: 48390340A/T
4-2: 48415992T/C
4-2: 48454581C/T
4-2: 48455217T/G
4-2: 48466729G/A
4-2: 48491292A/C
4-2: 48502063C/T
4-2: 48520757T/C
4-2: 48567270G/T
4-2: 48631480C/T
4-2: 48692532T/C
4-2: 48743517T/A
4-2: 48805750C/A
4-2: 48910451T/C
4-2: 48956250A/G
4-2: 48987927G/A
4-2: 49049560A/G
4-2: 49100373T/C
4-2: 49119722C/G
4-2: 49136985T/G
4-2: 49205371C/T
4-2: 49218193T/G
4-2: 49218611A/G
4-2: 49221760T/C
4-2: 49222513T/A
4-2: 49266757G/A
4-2: 49277166A/G
4-2: 49286133T/C
4-2: 49346241C/T
4-2: 49388082T/C
4-2: 49432018G/A
4-2: 49476733A/T
4-2: 49534655A/G
4-2: 49735985C/G
4-2: 49834302A/C
4-2: 49891242G/T
4-2: 49891460C/T
4-2: 49891986G/A
4-2: 49905732G/A
4-2: 49915641G/A
4-2: 49927470C/T
4-2: 49927684C/T
4-2: 50015815C/G
4-2: 50130300C/A
4-2: 50185541G/A
4-2: 50279086T/A
4-2: 50329436C/A
4-2: 50365576C/A
4-2: 50384617C/T
4-2: 50520855A/G
4-2: 50565775C/T
4-2: 50666015G/T
4-2: 50719651T/C
4-2: 50802594T/A
4-2: 50974126C/G
4-2: 51051446C/T
4-2: 51066433A/T
4-2: 51067039C/T
4-2: 51085498C/A
4-2: 51107004A/T
4-2: 51131615T/C
4-2: 51142970G/A
4-2: 51143528C/G
4-2: 51154699C/T
4-2: 51158167T/C
4-2: 51158215C/T
4-2: 51162670G/C
4-2: 51169419T/C
4-2: 51223114C/T
4-2: 51232231A/G
4-2: 51241403A/T
4-2: 51329957G/A
4-2: 51342850G/A
4-2: 51354082G/A
4-2: 51364781A/G
4-2: 51392599T/A
4-2: 51430776A/C
4-2: 51475729C/G
4-2: 51513701A/G
4-2: 51587859C/T
4-2: 51617380C/T
4-2: 51683756C/T
4-2: 51799547T/C
4-2: 51861340C/T
4-2: 51882750A/G
4-2: 51908952C/A
4-2: 51959465G/C
4-2: 51994999A/T
4-2: 52081766A/C
4-2: 52084091G/A
4-2: 52084623A/G
4-2: 52084658C/T
4-2: 52089891G/A
4-2: 52090038A/G
4-2: 52194424A/G
4-2: 52204667T/A
4-2: 52233538G/A
4-2: 52269107G/A
4-2: 52301744G/C
4-2: 52346425C/A
4-2: 52356323T/A
4-2: 52367263C/T
4-2: 52421702C/T
4-2: 52512043G/C
4-2: 52567916C/G
4-2: 52631215C/T
4-2: 52648882G/A
4-2: 52668377C/A
4-2: 52674531T/C
4-2: 52727245A/C
4-2: 52743622A/G
4-2: 52744699A/G
4-2: 52761100A/G
4-2: 52799603G/T
4-2: 52887053C/G
4-2: 52907394C/T
4-2: 52918933T/C
4-2: 52973496A/G
4-2: 53029823C/T
4-2: 53048615C/T
4-2: 53084502C/T
4-2: 53096489G/A
4-2: 53107392C/T
4-2: 53120542T/C
4-2: 53134022T/C
4-2: 53154352T/C
4-2: 53164597A/G
4-2: 53214055G/A
4-2: 53241329C/A
4-2: 53241588C/T
4-2: 53243079C/A
4-2: 53243320A/G
4-2: 53243470C/T
4-2: 53280356C/T
4-2: 53310471G/A
4-2: 53363657C/T
4-2: 53380701T/A
4-2: 53385439T/C
4-2: 53429789C/T
4-2: 53457487A/G
4-2: 53494040G/A
4-2: 53516349G/T
4-2: 53516677A/G
4-2: 53579958A/G
4-2: 53605129T/C
4-2: 53645301C/T
4-2: 53649562A/C
4-2: 53672934T/A
4-2: 53737916A/G
4-2: 53776585C/G
4-2: 53818674A/G
4-2: 53862206A/G
4-2: 53892110G/A
4-2: 53938512G/A
4-2: 54029224C/T
4-2: 54076461C/G
4-2: 54105056G/A
4-2: 54162344C/T
4-2: 54188237G/A
4-2: 54255586A/G
4-2: 54321114T/C
4-2: 54325933C/G
4-2: 54333067C/T
4-2: 54368860C/G
4-2: 54404575G/T
4-2: 54443235C/T
4-2: 54502857T/A
4-2: 54503139C/T
4-2: 54503152A/C
4-2: 54560610C/G
4-2: 54623138G/A
4-2: 54623425C/T
4-2: 54623452T/C
4-2: 54623470A/G
4-2: 54625121G/A
4-2: 54625276C/T
4-2: 54626104C/T
4-2: 54627278G/C
4-2: 54627411T/C
4-2: 54643638C/T
4-2: 54660261T/C
4-2: 54710793A/G
4-2: 54769350G/T
4-2: 54823116A/G
4-2: 54877345G/A
4-2: 54939756G/A
4-2: 54981589G/A
4-2: 54983317T/C
4-2: 55045653C/A
4-2: 55073137T/C
4-2: 55106107C/G
4-2: 55148597T/A
4-2: 55225291T/G
4-2: 55241473T/C
4-2: 55388947A/T
4-2: 55443325G/A
4-2: 55510457A/G
4-2: 55576542C/T
4-2: 55605585T/C
4-2: 55644618A/G
4-2: 55698605A/T
4-2: 55716338T/G
4-2: 55795970A/G
4-2: 55869013G/C
4-2: 55911992A/G
4-2: 55981191T/A
4-2: 56022746A/T
4-2: 56050578A/G
4-2: 56121153G/T
4-2: 56202879G/T
4-2: 56234842A/G
4-2: 56245212A/G
4-2: 56246018G/A
4-2: 56246306T/C
4-2: 56249366G/A
4-2: 56286909C/A
4-2: 56309603A/G
4-2: 56380829T/C
4-2: 56397736G/A
4-2: 56408766G/A
4-2: 56450170T/C
4-2: 56522889C/T
4-2: 56555539T/A
4-2: 56591819G/C
4-2: 56622422G/T
4-2: 56682549G/T
4-2: 56756961G/T
4-2: 56836445C/T
4-2: 56863128T/C
4-2: 57013172G/C
4-2: 57042695G/A
4-2: 57061552C/A
4-2: 57123880C/T
4-2: 57192567A/T
4-2: 57212620T/G
4-2: 57223115C/T
4-2: 57309381G/A
4-2: 57336032A/G
4-2: 57338131C/T
4-2: 57338207C/G
4-2: 57338239G/T
4-2: 57367872T/A
4-2: 57396345C/T
4-2: 57444376C/T
4-2: 57474346G/A
4-2: 57496942C/A
4-2: 57536995C/G
4-2: 57562758C/T
4-2: 57593250T/C
4-2: 57664097C/T
4-2: 57754395G/T
4-2: 57815593A/T
4-2: 57854127A/G
4-2: 58114534C/T
4-2: 58133237T/A
4-2: 58165969T/C
4-2: 58257830C/A
4-2: 58287296T/C
4-2: 58319749G/T
4-2: 58348382T/C
4-2: 58394140G/A
4-2: 58659042A/G
4-2: 58691366G/A
4-2: 58732942C/T
4-2: 58773296G/C
4-2: 58831051G/T
4-2: 58833875A/G
4-2: 58833964T/C
4-2: 58901146T/A
4-2: 58903011A/T
4-2: 58926827A/G
4-2: 58965712A/G
4-2: 59056706T/C
4-2: 59057831T/C
4-2: 59059370T/A
4-2: 59068375C/T
4-2: 59175376G/A
4-2: 59191983T/G
4-2: 59217432C/T
4-2: 59322311C/A
4-2: 59410591T/C
4-2: 59496086A/G
4-2: 59531777G/A
4-2: 59624039C/G
4-2: 59639737A/G
4-2: 59739711A/G
4-2: 59820670T/C
4-2: 59863515T/C
4-2: 59980881T/G
4-2: 60023349T/C
4-2: 60087320C/T
4-2: 60146212A/C
4-2: 60214922C/T
4-2: 60293902A/G
4-2: 60334943T/C
4-2: 60382881A/C
4-2: 60408165A/G
4-2: 60434019G/C
4-2: 60482455A/G
4-2: 60583447C/T
4-2: 60681380C/T
4-2: 60689439G/T
4-2: 60702077G/A
4-2: 60725297A/C
4-2: 60782522G/A
4-2: 60793783C/T
4-2: 60809177G/A
4-2: 60813544G/A
4-2: 60870581G/A
4-2: 60926811C/A
4-2: 60931234T/C
4-2: 60953549C/T
4-2: 60970102T/A
4-2: 60971652A/C
4-2: 61015683A/T
4-2: 61033817C/T
4-2: 61054113A/T
4-2: 61088883G/A
4-2: 61121641T/C
4-2: 61153915T/C
4-2: 61180026G/A
4-2: 61200002C/T
4-2: 61219207T/G
4-2: 61230569A/G
4-2: 61230792G/A
4-2: 61250837C/T
4-2: 61330466T/C
4-2: 61348210T/A
4-2: 61378214T/G
4-2: 61402956C/T
4-2: 61440073C/T
4-2: 61518184C/A
4-2: 61543150G/T
4-2: 61562052T/C
4-2: 61563479T/C
4-2: 61606207T/C
4-2: 61665080A/G
4-2: 61763429G/A
4-2: 61800136T/C
4-2: 61939655A/G
4-2: 62085967G/A
4-2: 62126712T/G
4-2: 62145121T/C
4-2: 62172212C/A
4-2: 62227335A/T
4-2: 62233810C/T
4-2: 62233946C/A
4-2: 62301150C/G
4-2: 62352222T/G
4-2: 62384065T/C
4-2: 62426139G/A
4-2: 62445968A/G
4-2: 62602798C/A
4-2: 62646889C/T
4-2: 62786782C/T
4-2: 62851035C/T
4-2: 62975655T/C
4-2: 63039427T/C
4-2: 63064703A/G
4-2: 63128677G/C
4-2: 63147520T/C
4-2: 63163909A/C
4-2: 63186470A/G
4-2: 63245014T/A
4-2: 63281398G/A
4-2: 63318136G/A
4-2: 63477762A/T
4-2: 63527009T/G
4-2: 63719565T/C
4-2: 63795246G/A
4-2: 63829477G/A
4-2: 63885427G/A
4-2: 63918760A/G
4-2: 63931505C/T
4-2: 64060172C/T
4-2: 64072996G/C
4-2: 64153034T/G
4-2: 64166421C/T
4-2: 64200159T/C
4-2: 64227732A/C
4-2: 64304698T/A
4-2: 64313073C/T
4-2: 64315806A/G
4-2: 64412865A/G
4-2: 64462149C/T
4-2: 64474235A/C
4-2: 64523046C/T
4-2: 64523332C/T
4-2: 64627218C/A
4-2: 64668747C/A
4-2: 64686895C/T
4-2: 64798948C/A
4-2: 64929441G/A
4-2: 64939967C/A
4-2: 64965085A/G
4-2: 65004876G/C
4-2: 65078405A/T
4-2: 65165786G/A
4-2: 65192693G/T
4-2: 65238038A/G
4-2: 65283136A/C
4-2: 65329649C/T
4-2: 65367557A/G
4-2: 65403854A/G
4-2: 65437477A/G
4-2: 65556604T/G
4-2: 65568270A/G
4-2: 65633875G/T
4-2: 65713835T/A
4-2: 65749756A/T
4-2: 65802076C/T
4-2: 65814617T/C
4-2: 65816566A/C
4-2: 65855713T/C
4-2: 66005645G/C
4-2: 66055620G/A
4-2: 66133198G/A
4-2: 66135354T/C
4-2: 66139218G/T
4-2: 66145104T/C
4-2: 66150584A/G
4-2: 66166302G/A
4-2: 66193384A/G
4-2: 66236973A/G
4-2: 66290403G/A
4-2: 66337762T/C
4-2: 66459539G/C
4-2: 66493753G/A
4-2: 66522421G/T
4-2: 66568688C/A
4-2: 66646933C/A
4-2: 66672070T/C
4-2: 66722460G/C
4-2: 66772916C/T
4-2: 66848391A/T
4-2: 66878491A/G
4-2: 67031610C/T
4-2: 67038581G/C
4-2: 67064607T/C
4-2: 67165683C/T
4-2: 67176746A/C
4-2: 67221967C/T
4-2: 67251747G/T
4-2: 67282574A/G
4-2: 67364846A/T
4-2: 67389878T/G
4-2: 67455969C/T
4-2: 67556419C/T
4-2: 67568952G/A
4-2: 67713459T/G
4-2: 67731388G/A
4-2: 67815505A/G
4-2: 67864419A/T
4-2: 67928595A/G
4-2: 68071014A/T
4-2: 68140246T/C
4-2: 68233002A/G
4-2: 68349395T/G
4-2: 68407667A/G
4-2: 68442532A/C
4-2: 68482847G/T
4-2: 68528075C/G
4-2: 68541858G/T
4-2: 68599762C/T
4-2: 68642995T/A
4-2: 68706944A/C
4-2: 68740583A/G
4-2: 68838535T/C
4-2: 68895709G/T
4-2: 69071056T/C
4-2: 69209678A/G
4-2: 69225055T/A
4-2: 69280236T/C
4-2: 69341430G/A
4-2: 69433208A/.
4-2: 69433211G/.
4-2: 69433215A/.
4-2: 69433216G/.
4-2: 69433220A/.
4-2: 69433236T/G
4-2: 69433345T/C
4-2: 69433357A/C
4-2: 69433362C/A
4-2: 69433365G/A
4-2: 69433366C/T
4-2: 69433368A/G
4-2: 69433378G/T
4-2: 69433384T/A
4-2: 69433395T/A
4-2: 69433396C/G
4-2: 69433403G/A
4-2: 69433404T/A
4-2: 69433410G/T
4-2: 69433413T/.
4-2: 69433415A/.
4-2: 69433417C/A
4-2: 69433437T/.
4-2: 69433476T/C
4-2: 69433777C/.
4-2: 69433780T/.
4-2: 69433802C/.
4-2: 69433805G/.
4-2: 69438072T/C
4-2: 69468951T/C
4-2: 69572274T/C
4-2: 69738617A/T
4-2: 69751110C/T
4-2: 69766280C/T
4-2: 69838326A/G
4-2: 69868242G/T
4-2: 69920519C/T
4-2: 69956581A/T
4-2: 70017327T/C
4-2: 70043798A/C
4-2: 70064228C/T
4-2: 70096130A/G
4-2: 70151827G/T
4-2: 70189485T/G
4-2: 70253929C/T
4-2: 70384121A/G
4-2: 70506523A/G
4-2: 70580089C/T
4-2: 70779560A/T
4-2: 70817979A/G
4-2: 70892824C/T
4-2: 70945331T/C
4-2: 71144746G/C
4-2: 71207280C/G
4-2: 71271428T/C
4-2: 71356022C/T
4-2: 71546346C/T
4-2: 71591526T/C
4-2: 71763596G/T
4-2: 72112041A/C
4-2: 72139406C/T
4-2: 72284693T/G
4-2: 72391344T/G
4-2: 72492670C/T
4-2: 72521684G/A
4-2: 72575152A/C
4-2: 72635820T/A
4-2: 72719352T/C
4-2: 72784144G/T
4-2: 72821816A/G
4-2: 72846964G/A
4-2: 72863356T/C
4-2: 73044681A/C
4-2: 73065854G/A
4-2: 73366011G/A
4-2: 73396103C/A
4-2: 73467323A/T
4-2: 73487400C/G
4-2: 73553379A/G
4-2: 73651705G/A
4-2: 73815792A/C
4-2: 73875884A/G
4-2: 73894977G/T
4-2: 73953426T/C
4-2: 73974176C/G
4-2: 73984418C/T
4-2: 73984427C/T
4-2: 74000323T/A
4-2: 74013644C/T
4-2: 74130140C/G
4-2: 74176285T/C
4-2: 74308749A/G
4-2: 74347135G/T
4-2: 74460766C/T
4-2: 74566929G/A
4-2: 74748626C/T
4-2: 74770477C/T
4-2: 74817726G/C
4-2: 74929119A/T
4-2: 74959660G/T
4-2: 74971944G/A
4-2: 74985131T/C
4-2: 74995390A/G
4-2: 74995589G/C
4-2: 75005031C/T
4-2: 75274932T/C
4-2: 75524601A/G
4-2: 75912496G/C
4-3: 85807G/A
4-3: 100680T/C
4-3: 242292C/T
4-3: 506873C/T
4-3: 689235G/A
4-3: 812834A/G
4-3: 1077438T/C
4-3: 1133741A/C
4-3: 1290953C/T
4-3: 1309190A/T
4-3: 1628792T/C
4-3: 1746868G/A
4-3: 1830608T/G
4-3: 1957739C/T
4-3: 2050163G/C
4-3: 2086212G/C
4-3: 2088224G/T
4-3: 2109425T/C
4-3: 2151448G/C
4-3: 2308204T/G
4-3: 2426592G/A
4-3: 2478754C/G
4-3: 2623499G/A
4-3: 2625673A/G
4-3: 2762570G/T
4-3: 2935673G/C
4-3: 3165233G/A
4-3: 3577537G/A
4-3: 3599878T/G
4-3: 3757370T/C
4-3: 3759666A/C
4-3: 3900120A/C
4-3: 3913761C/G
4-3: 3967607G/A
4-3: 4014593C/T
4-3: 4089607G/A
4-3: 4227612C/T
4-3: 4230496T/C
4-3: 4258465A/C
4-3: 4352731T/G
4-3: 4402989A/G
4-3: 4446617G/T
4-3: 4496811T/C
4-3: 4515814T/C
4-3: 4532184G/T
4-3: 4550658T/A
4-3: 4561350C/T
4-3: 4572605T/C
4-3: 4795292A/G
4-3: 4890833G/T
4-3: 4916015C/A
4-3: 4938458G/A
4-3: 4968640G/A
4-3: 4997616C/G
4-3: 5023535A/G
4-3: 5092692T/C
4-3: 5146060T/A
4-3: 5148972G/A
4-3: 5159064C/T
4-3: 5194463A/C
4-3: 5255309T/C
4-3: 5280751G/C
4-3: 5359414C/A
4-3: 5370209C/T
4-3: 5415436G/C
4-3: 5449188T/C
4-3: 5478005A/G
4-3: 5508240C/T
4-3: 5559597A/G
4-3: 5628058T/C
4-3: 5699099T/C
4-3: 5731329C/A
4-3: 5818225A/C
4-3: 5840785T/C
4-3: 5866040T/G
4-3: 5892497A/G
4-3: 5908880A/T
4-3: 5925719T/C
4-3: 5948567G/A
4-3: 5979063G/A
4-3: 6044252A/C
4-3: 6126945A/G
4-3: 6163486T/G
4-3: 6190417A/G
4-3: 6247081T/G
4-3: 6276683G/T
4-3: 6505973C/T
4-3: 6542847T/C
4-3: 6616590A/C
4-3: 6891268T/C
4-3: 6903775A/G
4-3: 6925814A/G
4-3: 6961461C/A
4-3: 7004265T/A
4-3: 7043462G/T
4-3: 7100717C/T
4-3: 7119822T/A
4-3: 7123176T/C
4-3: 7123366T/C
4-3: 7125981A/G
4-3: 7126653A/T
4-3: 7129774T/A
4-3: 7138679C/T
4-3: 7179987T/C
4-3: 7237908T/G
4-3: 7299770C/T
4-3: 7360824T/C
4-3: 7464473C/T
4-3: 7487717A/T
4-3: 7594108C/T
4-3: 7604969A/G
4-3: 7685533G/A
4-3: 7720837C/T
4-3: 7837089A/T
4-3: 8049680G/T
4-3: 8158777C/T
4-3: 8164886C/T
4-3: 8164997T/C
4-3: 8171091A/G
4-3: 8196695T/A
4-3: 8221782A/C
4-3: 8221962A/G
4-3: 8272420G/A
4-3: 8363256C/A
4-3: 8445264A/T
4-3: 8555420T/G
4-3: 8664344C/T
4-3: 8664916T/A
4-3: 8700623A/T
4-3: 8748967C/A
4-3: 8773576G/T
4-3: 8795038G/A
4-3: 8799079T/C
4-3: 8799107C/T
4-3: 8849160T/A
4-3: 8868610T/C
4-3: 8879191G/T
4-3: 8893429T/C
4-3: 8925206A/G
4-3: 8945215G/A
4-3: 8970657G/A
4-3: 9004717T/C
4-3: 9015909T/G
4-3: 9035517C/G
4-3: 9090447A/G
4-3: 9164446G/T
4-3: 9204346A/G
4-3: 9267575G/T
4-3: 9279870C/A
4-3: 9290973T/G
4-3: 9292448A/G
4-3: 9374741C/A
4-3: 9444951C/T
4-3: 9466429T/C
4-3: 9544495T/G
4-3: 9550672T/G
4-3: 9550889C/A
4-3: 9551222T/C
4-3: 9551772G/T
4-3: 9552228G/A
4-3: 9552741A/C
4-3: 9553564T/C
4-3: 9554543T/C
4-3: 9564990C/T
4-3: 9590011C/T
4-3: 9613108T/C
4-3: 9634431T/C
4-3: 9636323C/T
4-3: 9640063G/T
4-3: 9640780T/A
4-3: 9643051A/G
4-3: 9657113G/A
4-3: 9658505A/T
4-3: 9729475C/T
4-3: 9737572C/T
4-3: 9824790G/C
4-3: 9924046A/T
4-3: 9979088T/C
4-3: 10007075A/G
4-3: 10013186T/C
4-3: 10013854A/T
4-3: 10089750T/A
4-3: 10089867T/A
4-3: 10129089G/T
4-3: 10164089G/C
4-3: 10187379C/A
4-3: 10253284G/A
4-3: 10298012C/T
4-3: 10349912T/A
4-3: 10410571A/T
4-3: 10430445G/A
4-3: 10506122T/C
4-3: 10520590A/C
4-3: 10542701G/A
4-3: 10626004C/G
4-3: 10628556C/T
4-3: 10643339T/C
4-3: 10671695G/T
4-3: 10697042T/C
4-3: 10698802T/G
4-3: 10708751A/G
4-3: 10723327A/G
4-3: 10752862G/A
4-3: 10814898T/C
4-3: 10842169G/T
4-3: 10888168C/T
4-3: 10911257T/G
4-3: 10997930T/A
4-3: 11029228A/G
4-3: 11135299C/T
4-3: 11138236T/C
4-3: 11138274A/T
4-3: 11203783A/G
4-3: 11261295T/C
4-3: 11298744C/G
4-3: 11332523T/G
4-3: 11333176C/T
4-3: 11333218A/G
4-3: 11362002A/G
4-3: 11384775A/G
4-3: 11403868C/G
4-3: 11410078A/G
4-3: 11410163C/T
4-3: 11502182G/T
4-3: 11525470T/A
4-3: 11529572T/A
4-3: 11530468T/A
4-3: 11550193C/T
4-3: 11550294A/G
4-3: 11552350A/T
4-3: 11570945T/C
4-3: 11628432T/A
4-3: 11642443T/C
4-3: 11658328T/C
4-3: 11675674G/C
4-3: 11688282T/A
4-3: 11690945G/A
4-3: 11702358A/G
4-3: 11718443T/C
4-3: 11835589A/G
4-3: 11847326G/T
4-3: 11847371G/T
4-3: 11867939A/T
4-3: 11895556T/A
4-3: 12010881T/C
4-3: 12012243A/T
4-3: 12081494A/T
4-3: 12085317A/T
4-3: 12094248C/G
4-3: 12099331A/T
4-3: 12132300C/A
4-3: 12341024A/T
4-3: 12386808C/G
4-3: 12453885G/A
4-3: 12456290A/G
4-3: 12456659C/A
4-3: 12464245T/C
4-3: 12478594A/T
4-3: 12569083G/A
4-3: 12625847T/C
4-3: 12821838C/T
4-3: 12836029C/T
4-3: 12839033C/T
4-3: 12839079C/T
4-3: 12855540C/G
4-3: 12855654C/T
4-3: 12855676C/T
4-3: 12859024T/C
4-3: 12948369T/A
4-3: 13081366A/T
4-3: 13111817A/G
4-3: 13113982C/T
4-3: 13125897A/G
4-3: 13136058T/C
4-3: 13148322A/G
4-3: 13159667A/G
4-3: 13216046C/T
4-3: 13227836C/G
4-3: 13362137G/A
4-3: 13368630C/A
4-3: 13373924G/A
4-3: 13374435G/A
4-3: 13377087C/G
4-3: 13377141T/C
4-3: 13377376C/T
4-3: 13378162G/A
4-3: 13380122A/G
4-3: 13395568A/G
4-3: 13478552C/A
4-3: 13511215G/A
4-3: 13574080A/G
4-3: 13617068G/T
4-3: 13637594G/A
4-3: 13671663A/G
4-3: 13698007C/T
4-3: 13728508G/A
4-3: 13787205A/G
4-3: 13819496C/G
4-3: 13858267T/C
4-3: 13899944G/T
4-3: 13939849G/A
4-3: 13994107C/T
4-3: 14045263G/A
4-3: 14076572C/T
4-3: 14093726G/T
4-3: 14094194A/C
4-3: 14168721A/C
4-3: 14193131C/T
4-3: 14220419C/T
4-3: 14223570G/A
4-3: 14258000T/C
4-3: 14327857G/A
4-3: 14339405A/G
4-3: 14411283G/T
4-3: 14439531A/T
4-3: 14507990T/C
4-3: 14633846C/T
4-3: 14633969C/G
4-3: 14648558A/G
4-3: 14695696A/T
4-3: 14695702C/T
4-3: 14696253A/T
4-3: 14696862C/G
4-3: 14697050T/G
4-3: 14697636A/G
4-3: 14698664G/A
4-3: 14698755G/A
4-3: 14704736A/G
4-3: 14754990T/C
4-3: 14756234T/A
4-3: 14827060A/G
4-3: 14858731G/A
4-3: 14887206G/A
4-3: 14903230T/C
4-3: 14904084C/T
4-3: 14933251C/T
4-3: 14963230A/G
4-3: 15001942T/G
4-3: 15061514C/T
4-3: 15073810A/T
4-3: 15145828C/T
4-3: 15166028A/G
4-3: 15193412T/A
4-3: 15220890G/T
4-3: 15339246G/A
4-3: 15350623T/C
4-3: 15402013T/C
4-3: 15425562G/A
4-3: 15454648C/T
4-3: 15521411C/T
4-3: 15643820G/T
4-3: 15860302C/T
4-3: 15861388G/A
4-3: 15886694T/C
4-3: 15928145G/A
4-3: 16065568C/T
4-3: 16113025C/T
4-3: 16178575A/G
4-3: 16282783A/T
4-3: 16296154A/T
4-3: 16301323G/A
4-3: 16352065G/A
4-3: 16387666C/A
4-3: 16407025T/A
4-3: 16429316A/G
4-3: 16442945A/C
4-3: 16457857G/C
4-3: 16544366G/C
4-3: 16605614T/C
4-3: 16652191C/T
4-3: 16696567A/G
4-3: 16698749G/T
4-3: 16716031T/C
4-3: 16815507C/T
4-3: 16844926T/C
4-3: 16919174A/T
4-3: 16926825A/T
4-3: 16994852A/G
4-3: 17000858T/G
4-3: 17026791C/G
4-3: 17133991G/A
4-3: 17135087T/C
4-3: 17147577T/G
4-3: 17176745G/C
4-3: 17202679T/C
4-3: 17224873T/C
4-3: 17226025G/C
4-3: 17260284C/T
4-3: 17295585C/A
4-3: 17354126G/A
4-3: 17436062G/T
4-3: 17510456C/T
4-3: 17515406T/C
4-3: 17536895A/T
4-3: 17574864A/C
4-3: 17619594A/C
4-3: 17632754G/C
4-3: 17678211G/A
4-3: 17700816C/T
4-3: 17713239A/G
4-3: 17736102T/C
4-3: 17770782T/C
4-3: 17823388A/T
4-3: 17864026T/C
4-3: 17926223T/C
4-3: 18079317A/G
4-3: 18329220T/C
4-3: 18450613C/T
4-3: 18582336C/T
4-3: 18696471A/T
4-3: 18766229T/A
4-3: 18784282G/T
4-3: 18827052C/T
4-3: 18834517C/A
4-3: 18834806T/G
4-3: 18870444G/A
4-3: 18919128G/C
4-3: 19026995T/C
4-3: 19030406G/A
4-3: 19030423A/G
4-3: 19032152C/T
4-3: 19045055G/A
4-3: 19096419C/T
4-3: 19126951A/G
4-3: 19161912A/T
4-3: 19197784A/G
4-3: 19250038A/G
4-3: 19389429A/G
4-3: 19398226A/C
4-3: 19415074C/T
4-3: 19465451A/T
4-3: 19492304G/C
4-3: 19497799C/T
4-3: 19599768C/A
4-3: 19657187G/A
4-3: 19671204G/A
4-3: 19694532T/G
4-3: 19775773T/A
4-3: 19806858T/C
4-3: 19884978G/A
4-3: 19904622G/C
4-3: 19904747A/C
4-3: 19904775G/A
4-3: 19928829A/G
4-3: 19985179C/T
4-3: 19997740G/A
4-3: 20042415A/T
4-3: 20070348G/A
4-3: 20105481A/G
4-3: 20151593G/A
4-3: 20155312T/C
4-3: 20246952T/C
4-3: 20266160C/A
4-3: 20301171C/A
4-3: 20328892A/G
4-3: 20336952T/C
4-3: 20337473A/G
4-3: 20339262G/A
4-3: 20339326G/A
4-3: 20396110C/T
4-3: 20444507C/T
4-3: 20466205A/G
4-3: 20480087A/C
4-3: 20497208C/T
4-3: 20565317G/A
4-3: 20584949G/A
4-3: 20712221T/G
4-3: 20742018T/C
4-3: 20742935C/T
4-3: 20784384C/A
4-3: 20872289T/C
4-3: 20936823C/T
4-3: 20962003C/A
4-3: 20980918G/T
4-3: 21092671A/G
4-3: 21122331T/C
4-3: 21157676T/C
4-3: 21168164A/T
4-3: 21196494C/A
4-3: 21220975A/C
4-3: 21226539T/A
4-3: 21234001G/T
4-3: 21245902G/A
4-3: 21284936G/T
4-3: 21355074A/G
4-3: 21450882C/T
4-3: 21548388G/A
4-3: 21618025A/C
4-3: 21715405C/T
4-3: 21741294T/C
4-3: 21852106G/A
4-3: 21882680C/A
4-3: 21886009G/A
4-3: 21903568C/T
4-3: 21903989C/T
4-3: 21904200C/T
4-3: 21904901G/C
4-3: 21918038C/T
4-3: 21960230C/A
4-3: 22003777A/T
4-3: 22076207T/G
4-3: 22091628T/C
4-3: 22092356C/A
4-3: 22231969T/C
4-3: 22244988C/A
4-3: 22269025T/A
4-3: 22279719G/A
4-3: 22294336T/G
4-3: 22305977A/C
4-3: 22316433C/T
4-3: 22337653T/A
4-3: 22430122A/G
4-3: 22579084T/G
4-3: 22583804A/T
4-3: 22600722A/T
4-3: 22603517C/G
4-3: 22610709A/G
4-3: 22649596G/A
4-3: 22675196T/A
4-3: 22722281C/A
4-3: 22781179C/A
4-3: 22791801G/C
4-3: 22820788C/T
4-3: 22876084T/A
4-3: 22937003A/T
4-3: 22979118C/A
4-3: 23022625A/C
4-3: 23130444C/A
4-3: 23198672T/G
4-3: 23297764A/G
4-3: 23430696C/A
4-3: 23435098A/T
4-3: 23472361C/T
4-3: 23493628A/G
4-3: 23510222G/A
4-3: 23544420G/A
4-3: 23572166G/A
4-3: 23573006G/C
4-3: 23573032C/T
4-3: 23574877C/T
4-3: 23594202A/C
4-3: 23623063A/G
4-3: 23698524T/G
4-3: 23730672A/G
4-3: 23733907C/T
4-3: 23740953G/A
4-3: 23755421C/G
4-3: 23766280C/T
4-3: 23799118T/C
4-3: 23837716C/T
4-3: 23843271G/T
4-3: 23843388G/T
4-3: 23850543G/C
4-3: 23975213A/G
4-3: 23976809G/T
4-3: 24006406A/G
4-3: 24014868C/T
4-3: 24036190G/A
4-3: 24226402G/A
4-3: 24286706C/T
4-3: 24316063T/C
4-3: 24463175G/T
4-3: 24602308T/A
4-3: 24655074C/T
4-3: 24689712G/A
4-3: 24717325G/A
4-3: 24814137A/G
4-3: 24837620G/A
4-3: 24857058T/A
4-3: 24945115A/C
4-3: 24978311A/C
4-3: 25005404C/T
4-3: 25031706C/A
4-3: 25081032C/T
4-3: 25139446G/T
4-3: 25188034C/A
4-3: 25194180A/T
4-3: 25195322C/T
4-3: 25199601T/C
4-3: 25230270A/C
4-3: 25240145A/G
4-3: 25241969T/C
4-3: 25253550C/T
4-3: 25255071C/T
4-3: 25255167A/T
4-3: 25255222G/A
4-3: 25255550G/A
4-3: 25255619C/T
4-3: 25255655G/A
4-3: 25281884G/A
4-3: 25425665T/A
4-3: 25480161A/T
4-3: 25601832C/G
4-3: 25716055A/G
4-3: 25743773T/A
4-3: 26033635T/C
4-3: 26072417C/T
4-3: 26133082A/G
4-3: 26231583T/C
4-3: 26259746T/G
4-3: 26311883T/A
4-3: 26337398T/G
4-3: 26405391G/T
4-3: 26521775G/T
4-3: 26551013G/A
4-3: 26590422T/C
4-3: 26623862G/C
4-3: 26676529A/G
4-3: 26773689A/G
4-3: 26844709G/T
4-3: 26845734C/T
4-3: 27046595A/C
4-3: 27116914T/C
4-3: 27164025T/A
4-3: 27206353T/C
4-3: 27283816T/A
4-3: 27295193G/A
4-3: 27332334C/T
4-3: 27385975T/C
4-3: 27395179T/C
4-3: 27428381G/A
4-3: 27455126T/C
4-3: 27490685A/C
4-3: 27534877G/T
4-3: 27633634G/A
4-3: 27702991T/C
4-3: 27811892T/G
4-3: 28124267A/C
4-3: 28192099C/T
4-3: 28236578T/A
4-3: 28311955T/A
4-3: 28411882A/G
4-3: 28520013T/A
4-3: 28535340C/T
4-3: 28567035C/G
4-3: 28656172T/A
4-3: 28752356T/C
4-3: 28766681A/G
4-3: 28797335A/G
4-3: 28830547G/A
4-3: 28932036C/T
4-3: 28943110A/C
4-3: 28970539G/A
4-3: 29013875T/A
4-3: 29063659T/A
4-3: 29082948G/T
4-3: 29097897C/T
4-3: 29177007T/C
4-3: 29177033G/A
4-3: 29180039A/C
4-3: 29211025A/T
4-3: 29306166A/G
4-3: 29343829G/A
4-3: 29370590T/C
4-3: 29396497C/T
4-3: 29401984A/C
4-3: 29402014T/G
4-3: 29402017G/A
4-3: 29713978T/C
4-3: 29729042C/T
4-3: 29750816G/A
4-3: 29784590G/A
4-3: 29796102G/A
4-3: 29813402A/G
4-3: 29843479C/T
4-3: 29860920A/C
4-3: 29870944A/G
4-3: 29894391G/T
4-3: 29896654A/T
4-3: 30032711C/T
4-3: 30052390G/A
4-3: 30068688C/G
4-3: 30107157G/A
4-3: 30155765C/T
4-3: 30202857T/A
4-3: 30261832T/C
4-3: 30375366C/T
4-3: 30386134C/T
4-3: 30474556G/A
4-3: 30523296T/C
4-3: 30570832G/T
4-3: 30629949G/T
4-3: 30994257G/A
4-3: 31039590G/A
4-3: 31104331T/A
4-3: 31164453A/C
4-3: 31262367G/A
4-3: 31324152C/T
4-3: 31375089A/T
4-3: 31426598G/A
4-3: 31457957C/T
4-3: 31541520A/G
4-3: 31587022G/A
4-3: 31653958G/A
4-3: 31705963A/T
4-3: 31818691G/A
4-3: 31884145T/G
4-3: 32004184G/A
4-3: 32007987A/G
4-3: 32036736C/T
4-3: 32049128A/G
4-3: 32079410C/T
4-3: 32124762C/T
4-3: 32207722A/G
4-3: 32324201G/A
4-3: 32336967T/C
4-3: 32377858C/T
4-3: 32411362T/C
4-3: 32621229C/T
4-3: 32687473G/A
4-3: 32692439A/G
4-3: 32728127C/A
4-3: 32786774A/C
4-3: 33085029C/T
4-3: 33089788T/C
4-3: 33147364C/G
4-3: 33269316A/G
4-3: 33284680A/G
4-3: 33332046T/G
4-3: 33341865A/G
4-3: 33342178A/C
4-3: 33619591C/T
4-3: 33634750A/C
4-3: 33750546T/A
4-3: 33762014G/T
4-3: 33800698A/G
4-3: 33822343A/T
4-3: 33838427C/T
4-3: 33862105G/T
4-3: 34011918A/G
4-3: 34042209A/T
4-3: 34073848C/T
4-3: 34119557C/T
4-3: 34141818T/A
4-3: 34148488A/C
4-3: 34155870C/T
4-3: 34186607T/C
4-3: 34232086C/T
4-3: 34276648C/T
4-3: 34317573T/G
4-3: 34483698G/T
4-3: 34531069T/G
4-3: 34629085C/T
4-3: 34667955G/T
4-3: 34716615G/T
4-3: 34762629T/A
4-3: 34794997C/T
4-3: 34957255T/C
4-3: 34967969G/C
4-3: 34984481T/C
4-3: 35001503A/G
4-3: 35001521A/G
4-3: 35039313A/G
4-3: 35086086A/G
4-3: 35148160G/A
4-3: 35176416G/A
4-3: 35207473C/T
4-3: 35248701A/G
4-3: 35267589C/G
4-3: 35305190G/A
4-3: 35366819T/C
4-3: 35378506C/T
4-3: 35388710C/A
4-3: 35401398T/C
4-3: 35426069G/C
4-3: 35477971G/A
4-3: 35521017T/A
4-3: 35531618G/A
4-3: 35550051T/C
4-3: 35639143G/T
4-3: 35698256C/T
4-3: 35703677G/A
4-3: 35703870G/T
4-3: 35704115A/G
4-3: 35709038T/G
4-3: 35719497T/C
4-3: 35731283G/A
4-3: 35736688C/A
4-3: 35743660G/A
4-3: 35761986A/G
4-3: 35778622T/C
4-3: 35803079C/T
4-3: 35987193C/T
4-3: 36011246C/T
4-3: 36069264C/T
4-3: 36079644T/A
4-3: 36357875G/A
4-3: 36404277T/C
4-3: 36454420C/G
4-3: 36484945T/C
4-3: 36510486A/G
4-3: 36531775T/A
4-3: 36609914C/T
4-3: 36632474G/C
4-3: 36673241A/T
4-3: 36686223T/G
4-3: 36697434T/A
4-3: 36736317G/A
4-3: 36772976G/A
4-3: 36791236G/A
4-3: 36838472A/G
4-3: 36875058T/C
4-3: 36917003G/A
4-3: 36927940T/A
4-3: 36955436T/A
4-3: 36992062G/A
4-3: 37027434A/G
4-3: 37057689C/T
4-3: 37059238T/C
4-3: 37127130C/T
4-3: 37168390C/T
4-3: 37228931T/C
4-3: 37295885C/T
4-3: 37309266T/A
4-3: 37420355A/T
4-3: 37423616T/G
4-3: 37463996T/C
4-3: 37660366C/A
4-3: 37669879G/A
4-3: 37702479G/T
4-3: 37795378T/C
4-3: 37997397G/A
4-3: 38039594C/T
4-3: 38044323G/A
4-3: 38053435C/T
4-3: 38074347T/C
4-3: 38104802G/A
4-3: 38121329A/G
4-3: 38134949A/C
4-3: 38136343A/G
4-3: 38216440C/T
4-3: 38223614G/C
4-3: 38233992A/T
4-3: 38287751A/G
4-3: 38374888C/A
4-3: 38450061A/T
4-3: 38466449C/T
4-3: 38467235G/C
4-3: 38491130A/T
4-3: 38500889A/G
4-3: 38501306A/C
4-3: 38526653A/C
4-3: 38557615T/A
4-3: 38651620A/G
4-3: 38698411G/A
4-3: 38754535C/T
4-3: 38802589T/G
4-3: 38828324C/T
4-3: 38888452T/A
4-3: 38983435T/C
4-3: 38985360G/A
4-3: 38986719T/G
4-3: 38991201G/T
4-3: 38991202A/T
4-3: 38991227A/G
4-3: 38991228C/T
4-3: 38991246T/G
4-3: 38991261C/A
4-3: 38991267A/C
4-3: 38991294C/T
4-3: 38991314C/T
4-3: 38991453A/.
4-3: 38991483G/A
4-3: 38991507C/T
4-3: 38991510C/T
4-3: 38991547T/C
4-3: 38991579G/C
4-3: 38991597G/A
4-3: 38991615C/A
4-3: 38991619A/.
4-3: 38991627C/T
4-3: 38991645C/A
4-3: 38991654A/G
4-3: 38991660T/A
4-3: 39001541T/C
4-3: 39032183A/G
4-3: 39063081A/T
4-3: 39071141G/C
4-3: 39087500G/T
4-3: 39106951C/T
4-3: 39111435T/A
4-3: 39148883G/T
4-3: 39211947T/C
4-3: 39224081G/A
4-3: 39288010A/T
4-3: 39329885A/G
4-3: 39388098G/A
4-3: 39451953T/C
4-3: 39470889C/G
4-3: 39487924A/G
4-3: 39581817G/C
4-3: 39628220G/A
4-3: 39692761T/A
4-3: 39722089A/G
4-3: 39766409A/G
4-3: 39797085A/G
4-3: 39855269A/T
4-3: 39901043T/C
4-3: 39949271G/T
4-3: 40013079C/G
4-3: 40015604C/A
4-3: 40059782G/A
4-3: 40083200G/T
4-3: 40095650C/G
4-3: 40200640T/C
4-3: 40202853G/T
4-3: 40220353T/G
4-3: 40241244T/G
4-3: 40243250G/A
4-3: 40340796G/C
4-3: 40406732A/G
4-3: 40468207G/A
4-3: 40470595C/T
4-3: 40481032T/C
4-3: 40495805G/A
4-3: 40515183A/G
4-3: 40623312A/G
4-3: 40644168T/C
4-3: 40706725A/G
4-3: 40789857A/G
4-3: 40816606T/C
4-3: 40869280A/G
4-3: 40922609C/T
4-3: 40991035T/A
4-3: 41019683T/A
4-3: 41063413G/T
4-3: 41110093A/G
4-3: 41153317T/C
4-3: 41215233A/G
4-3: 41256504C/T
4-3: 41347982C/T
4-3: 41429386A/G
4-3: 41510609C/T
4-3: 41534639T/C
4-3: 41570017C/T
4-3: 41641124T/C
4-3: 41642151G/T
4-3: 41684909A/G
4-3: 41705269C/A
4-3: 41734186C/A
4-3: 41796627A/C
4-3: 41807699T/C
4-3: 41839149A/G
4-3: 41852026T/A
4-3: 41999537T/C
4-3: 42108525T/C
4-3: 42111613G/A
4-3: 42123477A/G
4-3: 42126599A/T
4-3: 42126826T/G
4-3: 42141436T/A
4-3: 42143439A/G
4-3: 42154092T/G
4-3: 42203722A/G
4-3: 42238938C/T
4-3: 42281714T/C
4-3: 42315219T/C
4-3: 42361855A/G
4-3: 42380274G/A
4-3: 42406557G/C
4-3: 42497510C/A
4-3: 42556388T/C
4-3: 42596133T/A
4-3: 42618784C/T
4-3: 42618892T/C
4-3: 42653509T/C
4-3: 42666519A/G
4-3: 42708743C/T
4-3: 42796676C/T
4-3: 42829309A/C
4-3: 42857488A/G
4-3: 42881070C/T
4-3: 43019966G/T
4-3: 43030351C/T
4-3: 43098384T/C
4-3: 43125109A/G
4-3: 43156284C/G
4-3: 43167085G/A
4-3: 43207139A/G
4-3: 43302290G/A
4-3: 43468492G/T
4-3: 43496289G/C
4-3: 43497453G/C
4-3: 43509446C/T
4-3: 43545156T/A
4-3: 43609406G/T
4-3: 43649532T/C
4-3: 43679774C/T
4-3: 43758129T/G
4-3: 43771861A/T
4-3: 43793408C/G
4-3: 43841906A/T
4-3: 43869184G/A
4-3: 43887985T/C
4-3: 43937569G/A
4-3: 43953354A/T
4-3: 43989747G/A
4-3: 44088741G/C
4-3: 44102123A/G
4-3: 44125885G/A
4-3: 44136320C/T
4-3: 44160278C/A
4-3: 44215956T/C
4-3: 44270437C/G
4-3: 44271162A/G
4-3: 44271469A/G
4-3: 44307238G/T
4-3: 44320394T/C
4-3: 44351874G/T
4-3: 44370355C/T
4-3: 44384108A/G
4-3: 44386045C/T
4-3: 44420268G/A
4-3: 44474068C/T
4-3: 44538106G/A
4-3: 44571163T/C
4-3: 44600934A/T
4-3: 44649829C/T
4-3: 44670177G/A
4-3: 44720424C/T
4-3: 44720591T/C
4-3: 44720724G/A
4-3: 44742397C/T
4-3: 44753717G/A
4-3: 44848680G/A
4-3: 44859861C/T
4-3: 44874253C/G
4-3: 44902456A/G
4-3: 44935418A/T
4-3: 44992901T/G
4-3: 45021472A/G
4-3: 45081253G/T
4-3: 45114429A/T
4-3: 45160129A/G
4-3: 45173301A/G
4-3: 45188766T/C
4-3: 45188867A/C
4-3: 45188952C/A
4-3: 45199771T/C
4-3: 45200050T/A
4-3: 45236070T/C
4-3: 45239398C/A
4-3: 45253105T/C
4-3: 45284811T/A
4-3: 45331797G/A
4-3: 45342077A/T
4-3: 45356655A/G
4-3: 45388589T/A
4-3: 45399268A/T
4-3: 45440313C/A
4-3: 45488711T/C
4-3: 45555827T/A
4-3: 45560652C/A
4-3: 45566310C/A
4-3: 45594265C/T
4-3: 45643081A/G
4-3: 45680050G/A
4-3: 45712757C/T
4-3: 45748375T/A
4-3: 45795619A/C
4-3: 45831110A/T
4-3: 45853378A/G
4-3: 45887099C/T
4-3: 45979735G/T
4-3: 46053664G/A
4-3: 46113338T/C
4-3: 46145591T/C
4-3: 46214334A/C
4-3: 46277599G/T
4-3: 46300259T/C
4-3: 46317112G/A
4-3: 46344421T/C
4-3: 46370363A/G
4-3: 46409282T/A
4-3: 46434438A/G
4-3: 46511088T/C
4-3: 46514329T/A
4-3: 46519307G/A
4-3: 46519325T/A
4-3: 46522092A/G
4-3: 46547727G/T
4-3: 46560096C/G
4-3: 46723627A/G
4-3: 46734774T/C
4-3: 46750379T/C
4-3: 46760812T/C
4-3: 46806280A/C
4-3: 46915676T/C
4-3: 47024887C/A
4-3: 47069352G/A
4-3: 47086995C/T
4-3: 47102026A/G
4-3: 47136336T/C
4-3: 47148799G/C
4-3: 47195526G/A
4-3: 47214149G/T
4-3: 47244576A/T
4-3: 47288034G/A
4-3: 47318266C/T
4-3: 47347797A/T
4-3: 47389296G/A
4-3: 47469675A/G
4-3: 47528476G/A
4-3: 47571474A/G
4-3: 47608070C/T
4-3: 47639482G/A
4-3: 47677211G/C
4-3: 47763465T/C
4-3: 47865227A/G
4-3: 47996723A/G
4-3: 48000914A/T
4-3: 48025929C/G
4-3: 48071909T/A
4-3: 48113571A/G
4-3: 48170602A/T
4-3: 48182480A/G
4-3: 48245823T/A
4-3: 48272302C/T
4-3: 48302053G/C
4-3: 48311120A/T
4-3: 48322621T/C
4-3: 48354512G/C
4-3: 48386490A/G
4-3: 48552671T/C
4-3: 48572786T/C
4-3: 48673343G/A
4-3: 48713518A/G
4-3: 48788999G/C
4-3: 48909154C/T
4-3: 48917896T/C
4-3: 48920647G/A
4-3: 48941605C/T
4-3: 48955527C/T
4-3: 48983440C/T
4-3: 49019198A/G
4-3: 49031908G/T
4-3: 49047453A/G
4-3: 49157018A/G
4-3: 49161644C/T
4-3: 49196172A/T
4-3: 49244378T/G
4-3: 49295768T/A
4-3: 49321484A/G
4-3: 49325180T/A
4-3: 49401888A/G
4-3: 49403136A/T
4-3: 49407018T/C
4-3: 49469514A/T
4-3: 49587692A/G
4-3: 49588233A/G
4-3: 49588251A/C
4-3: 49600311A/T
4-3: 49700157G/T
4-3: 49710589C/T
4-3: 49742189C/T
4-3: 49795638C/A
4-3: 49847269C/T
4-3: 49886599C/T
4-3: 49970243G/T
4-3: 50128966T/C
4-3: 50135598A/T
4-3: 50147989C/T
4-3: 50250436G/A
4-3: 50304304T/A
4-3: 50318829C/G
4-3: 50368101C/T
4-3: 50378472A/C
4-3: 50395167G/A
4-3: 50427313A/T
4-3: 50444300G/A
4-3: 50473713G/T
4-3: 50497705T/C
4-3: 50591612C/T
4-3: 50619721A/T
4-3: 50671510A/C
4-3: 50691262T/C
4-3: 50861183A/G
4-3: 50874202T/G
4-3: 50898070G/C
4-3: 50926763T/C
4-3: 50987232C/T
4-3: 51019973T/A
4-3: 51106321A/C
4-3: 51184259G/A
4-3: 51210353G/A
4-3: 51254122G/A
4-3: 51296990A/G
4-3: 51310377T/C
4-3: 51337087A/T
4-3: 51394464T/C
4-3: 51410006G/A
4-3: 51482131G/A
4-3: 51678721C/T
4-3: 51805113C/T
4-3: 51887120A/G
4-3: 51963380G/T
4-3: 51975432G/A
4-3: 52030671G/A
4-3: 52059392G/A
4-3: 52123356G/C
4-3: 52207559T/C
4-3: 52271009T/C
4-3: 52339215A/C
4-3: 52397167G/A
4-3: 52424869C/T
4-3: 52490029T/C
4-3: 52503662A/G
4-3: 52535029C/T
4-3: 52590560A/C
4-3: 52614353C/G
4-3: 52761304T/G
4-3: 52884848T/C
4-3: 52956334G/A
4-3: 52992029T/A
4-3: 53023663T/C
4-3: 53066664C/A
4-3: 53119023A/G
4-3: 53131357A/G
4-3: 53147980C/T
4-3: 53167508C/T
4-3: 53179992G/A
4-3: 53192405T/C
4-3: 53206744A/C
4-3: 53309884T/C
4-3: 53353261G/A
4-3: 53405817T/C
4-3: 53441269G/A
4-3: 53480549C/A
4-3: 53519215T/G
4-3: 53549016T/G
4-3: 53615407T/C
4-3: 53642983A/G
4-3: 53682287G/A
4-3: 53730753T/C
4-3: 53795802G/A
4-3: 53822979C/T
4-3: 53918978A/T
4-3: 53929973G/A
4-3: 53989430C/G
4-3: 54033769C/T
4-3: 54067949C/T
4-3: 54098483G/A
4-3: 54160387G/A
4-3: 54257071T/A
4-3: 54297455C/T
4-3: 54426856C/T
4-3: 54440628A/G
4-3: 54461202T/C
4-3: 54517416A/G
4-3: 54671034A/G
4-3: 54707931G/A
4-3: 54788214A/G
4-3: 54838987A/G
4-3: 54849378G/A
4-3: 54892234G/T
4-3: 54907886T/C
4-3: 55005422T/A
4-3: 55045852A/T
4-3: 55046168G/A
4-3: 55047060A/G
4-3: 55102059C/T
4-3: 55146011G/A
4-3: 55150719T/A
4-3: 55179193A/C
4-3: 55245062A/T
4-3: 55249240C/T
4-3: 55255519T/G
4-3: 55257348A/G
4-3: 55275313C/T
4-3: 55288516G/C
4-3: 55316311C/T
4-3: 55398050G/T
4-3: 55412797A/T
4-3: 55424108C/T
4-3: 55435335T/C
4-3: 55450804T/A
4-3: 55455388A/C
4-3: 55472807C/T
4-3: 55499161T/G
4-3: 55534650T/C
4-3: 55542881T/C
4-3: 55545322G/A
4-3: 55557707C/G
4-3: 55567836T/C
4-3: 55579704T/C
4-3: 55660510G/A
4-3: 55762496A/C
4-3: 55800560A/T
4-3: 55844826A/T
4-3: 55886743T/C
4-3: 55924699A/C
4-3: 55938102T/C
4-3: 56006676G/T
4-3: 56055261A/C
4-3: 56094590C/A
4-3: 56147212T/A
4-3: 56186706C/T
4-3: 56236272T/G
4-3: 56236297C/T
4-3: 56257048G/A
4-3: 56355674C/A
4-3: 56372366A/T
4-3: 56569932A/T
4-3: 56601652A/T
4-3: 56714905C/T
4-3: 56748908C/T
4-3: 56748963C/G
4-3: 56808897G/C
4-3: 56819589C/T
4-3: 56832717A/C
4-3: 56919516A/T
4-3: 56945164T/G
4-3: 56980500T/G
4-3: 57031168G/C
4-3: 57033432G/C
4-3: 57034703T/G
4-3: 57034724T/A
4-3: 57035675G/A
4-3: 57035774A/T
4-3: 57047005A/G
4-3: 57047276A/C
4-3: 57047664A/G
4-3: 57059706G/A
4-3: 57070473A/T
4-3: 57072137G/.
4-3: 57094206T/C
4-3: 57114569C/A
4-3: 57521214T/A
4-3: 57621033G/A
4-3: 57668680G/T
4-3: 57685885A/G
4-3: 57696325C/A
4-3: 57713793A/G
4-3: 57744726C/T
4-3: 57775507G/T
4-3: 57802180T/G
4-3: 57877616T/C
4-3: 57991975T/C
4-3: 58075691A/T
4-3: 58119683T/A
4-3: 58151129A/T
4-3: 58216174G/A
4-3: 58234762A/G
4-3: 58252224T/C
4-3: 58300828A/G
4-3: 58312872G/A
4-3: 58366105A/G
4-3: 58401255A/G
4-3: 58412831C/A
4-3: 58435613C/T
4-3: 58483441A/C
4-3: 58514557A/G
4-3: 58552861A/C
4-3: 58572606A/G
4-3: 58655741A/T
4-3: 58711906C/T
4-3: 58743856C/T
4-3: 58763649G/T
4-3: 58792474T/C
4-3: 58822264A/T
4-3: 58919896A/T
4-3: 58990583G/A
4-3: 59052741A/G
4-3: 59108875A/G
4-3: 59166744T/C
4-3: 59198291C/T
4-3: 59230417T/G
4-3: 59325194C/T
4-3: 59383851G/A
4-3: 59410657A/G
4-3: 59460367A/G
4-3: 59460664A/T
4-3: 59476087G/A
4-3: 59488430C/T
4-3: 59499369C/T
4-3: 59575360A/C
4-3: 59588170T/C
4-3: 59632269G/A
4-3: 59671906A/G
4-3: 59753310A/G
4-3: 59783357G/T
4-3: 59809047C/T
4-3: 59856033C/T
4-3: 59887371C/A
4-3: 59899300C/T
4-3: 59915391C/T
4-3: 59926057A/G
4-3: 60010675C/T
4-3: 60027599T/C
4-3: 60046492A/G
4-3: 60079163A/G
4-3: 60148901T/C
4-3: 60185525G/T
4-3: 60219112C/A
4-3: 60273698C/T
4-3: 60283855A/G
4-3: 60295922C/T
4-3: 60296434G/A
4-3: 60297529G/C
4-3: 60297665T/C
4-3: 60301168A/T
4-3: 60308950T/C
4-3: 60320314T/C
4-3: 60356013C/T
4-3: 60409514T/A
4-3: 60543292T/G
4-3: 60565705T/C
4-3: 60625804A/C
4-3: 60716166T/C
4-3: 60767699G/A
4-3: 60836921A/C
4-3: 60882635C/T
4-3: 60956902T/C
4-3: 61072972G/A
4-3: 61179634A/T
4-3: 61246993C/T
4-3: 61281343C/G
4-3: 61353265A/T
4-3: 61407899C/T
4-3: 61440863C/T
4-3: 61495164C/T
4-3: 61516382A/T
4-3: 61549344T/C
4-3: 61636021C/T
4-3: 61675770G/A
4-3: 61736263T/C
4-3: 61815853A/G
4-3: 61849655C/T
4-3: 61909710T/A
4-3: 61937299C/T
4-3: 61970882T/C
4-3: 62018522C/A
4-3: 62096688A/G
4-3: 62127013T/C
4-3: 62233188T/C
4-3: 62300341T/C
4-3: 62394765A/T
4-3: 62480694T/C
4-3: 62555037G/T
4-3: 62681770A/G
4-3: 62718572C/T
4-3: 62993058A/G
4-3: 63010000C/T
4-3: 63137929G/T
4-3: 63223596T/G
4-3: 63271472C/T
4-3: 63417238C/T
4-3: 63540374G/A
4-3: 63576134G/A
4-3: 63683915C/T
4-3: 63736295T/C
4-3: 63765416A/C
4-3: 63808204C/T
4-3: 63841322G/A
4-3: 63886794C/T
4-3: 64017587G/A
4-3: 64087063C/A
4-3: 64122488T/C
4-3: 64249242A/T
4-3: 64270639G/A
4-3: 64285299A/T
4-3: 64438876C/T
4-3: 64584120A/G
4-3: 64650150A/T
4-3: 64764335A/G
4-3: 64842744C/T
4-3: 64929453T/A
4-3: 64942248C/T
4-3: 65081834G/A
4-3: 65095948C/T
4-3: 65099426A/G
4-3: 65110054A/C
4-3: 65136754T/C
4-3: 65245673C/A
4-3: 65282511C/T
4-3: 65360980G/A
4-3: 65412776G/A
4-3: 65471208G/T
4-3: 65530321G/C
4-3: 65564711G/A
4-3: 65596062T/A
4-3: 65664277C/T
4-3: 65692659G/A
4-3: 65752772T/A
4-3: 65841200G/T
4-3: 65853223A/G
4-3: 65856434T/C
4-3: 65863700C/T
4-3: 65888493C/T
4-3: 65969569G/A
4-3: 65985038C/T
4-3: 66060550G/T
4-3: 66118109G/A
4-3: 66138734T/C
4-3: 66261829G/A
4-3: 66354973T/C
4-3: 66366622T/A
4-3: 66388390C/T
4-3: 66471268C/T
4-3: 66515760C/A
4-3: 66575447C/T
4-3: 66638107G/T
4-3: 66668729C/G
4-3: 66738892T/G
4-3: 66752858T/C
4-3: 66792375G/A
4-3: 66914574A/G
4-3: 67028909A/G
4-3: 67032384A/T
4-3: 67092475G/A
4-3: 67225495G/C
4-3: 67237769C/T
4-3: 67262858T/G
4-3: 67364935C/T
4-3: 67440134A/C
4-3: 67451765T/C
4-3: 67648505A/G
4-3: 67669080C/T
4-3: 67725809C/T
4-3: 67745370G/C
4-3: 67757222G/C
4-3: 67767646G/A
4-3: 67792083G/A
4-3: 67826125A/C
4-3: 67908375A/C
4-3: 67984586G/A
4-3: 68071622T/A
4-3: 68076827C/T
4-3: 68076831T/C
4-3: 68076837T/A
4-3: 68076880G/A
4-3: 68076881C/G
4-3: 68076897C/T
4-3: 68076912A/T
4-3: 68076922T/A
4-3: 68076952G/.
4-3: 68076953A/G
4-3: 68076967G/C
4-3: 68076979C/T
4-3: 68076995T/C
4-3: 68077020T/A
4-3: 68077027C/T
4-3: 68077037G/A
4-3: 68077051A/G
4-3: 68077060G/A
4-3: 68077066C/.
4-3: 68077067C/T
4-3: 68077068T/G
4-3: 68077070C/T
4-3: 68077074T/A
4-3: 68077075G/A
4-3: 68077078A/G
4-3: 68077080G/C
4-3: 68077087G/A
4-3: 68077096G/T
4-3: 68077101T/C
4-3: 68077110G/T
4-3: 68077116A/C
4-3: 68077142A/G
4-3: 68077153G/C
4-3: 68077157A/T
4-3: 68077170A/T
4-3: 68077181A/T
4-3: 68077208A/T
4-3: 68077227G/T
4-3: 68077243G/A
4-3: 68077260A/C
4-3: 68077266T/C
4-3: 68077273A/T
4-3: 68077274T/A
4-3: 68077280C/.
4-3: 68077288A/G
4-3: 68077292T/C
4-3: 68077294T/A
4-3: 68077308G/C
4-3: 68077324G/A
4-3: 68077334A/C
4-3: 68077339A/T
4-3: 68077341G/T
4-3: 68077375G/.
4-3: 68077385T/A
4-3: 68077389T/A
4-3: 68077406G/A
4-3: 68077552G/.
4-3: 68077587C/T
4-3: 68077589A/C
4-3: 68077593T/G
4-3: 68077648C/A
4-3: 68077781A/G
4-3: 68077926C/T
4-3: 68078040G/.
4-3: 68078043C/.
4-3: 68078059C/.
4-3: 68078062G/.
4-3: 68078064T/.
4-3: 68078070T/C
4-3: 68078071A/.
4-3: 68078108T/.
4-3: 68078109C/A
4-3: 68078115T/G
4-3: 68078118T/C
4-3: 68078341A/G
4-3: 68078385G/A
4-3: 68078386T/C
4-3: 68078387G/T
4-3: 68078396C/.
4-3: 68078436T/.
4-3: 68078457G/C
4-3: 68104578A/G
4-3: 68154274T/A
4-3: 68215942A/G
4-3: 68282698A/C
4-3: 68377467C/A
4-3: 68544562A/G
4-3: 68549177T/C
4-3: 68591957C/T
4-3: 68609679C/T
4-3: 68674239C/T
4-3: 68776390C/G
4-3: 68870813T/C
4-3: 68940749T/C
4-3: 69034015G/A
4-3: 69085271A/G
4-3: 69129015G/A
4-3: 69235788G/A
4-3: 69247078C/T
4-3: 69317985C/T
4-3: 69355644A/G
4-3: 69405709G/A
4-3: 69541073G/A
4-3: 69634374G/A
4-3: 69660391A/T
4-3: 69747319T/G
4-3: 69783959G/C
4-3: 69924839A/G
4-3: 69983680A/T
4-3: 69993837G/A
4-3: 70024430C/A
4-3: 70058605C/T
4-3: 70109322C/T
4-3: 70111908A/T
4-3: 70196564A/G
4-3: 70229192A/G
4-3: 70253429T/C
4-3: 70256191C/T
4-3: 70285895G/A
4-3: 70342532G/A
4-3: 70479415C/G
4-3: 70524563G/C
4-3: 70607465A/C
4-3: 70801020T/C
4-3: 70956013C/T
4-3: 71168506T/C
4-3: 71233391C/T
4-3: 71361500G/A
4-3: 71389834C/T
4-3: 71391509G/A
4-3: 71415116A/G
4-3: 71429689T/C
4-3: 71625248G/A
4-3: 71676953C/T
4-3: 71803227A/G
4-3: 72035177G/A
4-3: 72037265C/T
4-3: 72065025G/A
4-3: 72128138T/A
4-3: 72192781T/G
4-3: 72204039A/G
4-3: 72227869C/T
4-3: 72240792G/A
4-3: 72242451T/C
4-3: 72267851A/T
4-3: 72285439G/A
4-3: 72295470G/C
4-3: 72296791C/T
4-3: 72339702A/G
4-3: 72397076A/T
4-3: 72551861C/T
4-3: 72579647C/G
4-3: 72682635G/A
4-3: 72693956G/A
4-3: 72700742C/A
4-3: 72771662A/G
4-3: 72920071G/A
4-3: 73068694C/T
4-3: 73353551A/G
4-3: 73501691G/A
4-3: 73502754A/T
4-3: 73534633C/G
4-3: 73582939G/A
4-3: 73779862C/T
4-3: 73881862A/G
4-3: 74021480A/G
4-3: 74033841A/T
4-3: 74036284T/C
4-3: 74211180T/C
4-3: 74212140G/A
4-3: 74212187C/T
4-3: 74222535A/C
4-3: 74222747A/T
4-3: 74222798T/G
4-3: 74222801T/C
4-3: 74224759A/G
4-3: 74224762T/C
4-3: 74224764C/T
4-3: 74433954T/A
4-4: 279C/T
4-4: 147579C/T
4-4: 330853T/C
4-4: 338460A/C
4-4: 469529C/G
4-4: 652311T/C
4-4: 746734A/C
4-4: 983036T/A
4-4: 1066044C/G
4-4: 1179171G/A
4-4: 1264575G/T
4-4: 1305461T/C
4-4: 1347480C/T
4-4: 1854380C/T
4-4: 1877785G/T
4-4: 2319874T/A
4-4: 2319883T/A
4-4: 2320134T/C
4-4: 2345606A/C
4-4: 2526762T/A
4-4: 2610720A/C
4-4: 2734192G/A
4-4: 2779921T/C
4-4: 2810752T/G
4-4: 2848757C/T
4-4: 2873030T/C
4-4: 2894982G/A
4-4: 2917387T/G
4-4: 2935681A/T
4-4: 2962937G/C
4-4: 3123546T/C
4-4: 3136831T/C
4-4: 3247520C/A
4-4: 3301849G/A
4-4: 3398522A/G
4-4: 3426284A/G
4-4: 3461087G/A
4-4: 3490727A/G
4-4: 3502685G/A
4-4: 3546594C/T
4-4: 3560836C/A
4-4: 3561102T/C
4-4: 3626464T/C
4-4: 3654255C/T
4-4: 3655013T/C
4-4: 3743859G/A
4-4: 3770521G/A
4-4: 3795849C/T
4-4: 3819606T/G
4-4: 3835920G/T
4-4: 3926805G/T
4-4: 3982368C/T
4-4: 4034053G/T
4-4: 4088109G/A
4-4: 4238411C/A
4-4: 4249036G/A
4-4: 4260224T/G
4-4: 4272770T/A
4-4: 4283100C/T
4-4: 4296221A/G
4-4: 4358879A/G
4-4: 4534373T/C
4-4: 4540055G/A
4-4: 4615851G/A
4-4: 4695672A/G
4-4: 4699938T/C
4-4: 4759540A/G
4-4: 4776908C/T
4-4: 4849565T/G
4-4: 4901817G/A
4-4: 4931182C/T
4-4: 4963488G/T
4-4: 4976778C/T
4-4: 4979944C/T
4-4: 5048924G/A
4-4: 5103030C/T
4-4: 5117551G/T
4-4: 5118924G/C
4-4: 5118950T/G
4-4: 5143237A/G
4-4: 5229693A/T
4-4: 5263267A/G
4-4: 5318461C/A
4-4: 5509250G/A
4-4: 5612203T/C
4-4: 5683520T/G
4-4: 5718291A/C
4-4: 5719059A/G
4-4: 5790356T/C
4-4: 5883257C/G
4-4: 5907553T/A
4-4: 5993387G/C
4-4: 6149638T/C
4-4: 6236538A/G
4-4: 6251816C/G
4-4: 6305806T/C
4-4: 6337575T/A
4-4: 6472124T/C
4-4: 6474459A/G
4-4: 6485039G/T
4-4: 6493359C/T
4-4: 6513359C/T
4-4: 6514211T/C
4-4: 6526754T/A
4-4: 6587263C/T
4-4: 6615002C/T
4-4: 6638311C/T
4-4: 6721839G/A
4-4: 6770618G/A
4-4: 6991824C/T
4-4: 7006546G/A
4-4: 7093151T/G
4-4: 7155576A/T
4-4: 7195087A/T
4-4: 7224876T/A
4-4: 7232599G/C
4-4: 7334443G/C
4-4: 7366166C/G
4-4: 7419212G/T
4-4: 7496158A/C
4-4: 7527129G/A
4-4: 7576670C/T
4-4: 7678304G/A
4-4: 7758494A/T
4-4: 7787238A/G
4-4: 7886532G/A
4-4: 7919430G/A
4-4: 7983805G/A
4-4: 8128897A/G
4-4: 8168028T/C
4-4: 8180337G/T
4-4: 8244701A/T
4-4: 8245217A/C
4-4: 8245254G/A
4-4: 8246089T/C
4-4: 8254885G/T
4-4: 8263079A/G
4-4: 8269994T/C
4-4: 8282388A/T
4-4: 8297686C/A
4-4: 8309364A/G
4-4: 8311030A/G
4-4: 8369092C/T
4-4: 8513530T/C
4-4: 8663942A/G
4-4: 8675618C/T
4-4: 8707299A/G
4-4: 8757168A/T
4-4: 8826321C/T
4-4: 8840330A/G
4-4: 8890070T/A
4-4: 8950926A/C
4-4: 8962809G/T
4-4: 9025633G/C
4-4: 9025792T/C
4-4: 9073346T/A
4-4: 9090166G/T
4-4: 9105369G/A
4-4: 9156114C/T
4-4: 9189236A/C
4-4: 9200818A/G
4-4: 9262627T/C
4-4: 9310285C/G
4-4: 9310286A/G
4-4: 9352414C/A
4-4: 9377857C/T
4-4: 9392088C/T
4-4: 9471526G/A
4-4: 9484673A/G
4-4: 9484704T/C
4-4: 9513575A/T
4-4: 9560097G/T
4-4: 9658436G/A
4-4: 9936192T/C
4-4: 9937650A/G
4-4: 9970283G/A
4-4: 10002160A/G
4-4: 10057651T/C
4-4: 10106716T/C
4-4: 10178345A/T
4-4: 10245180G/T
4-4: 10299484T/G
4-4: 10325009A/T
4-4: 10370671C/G
4-4: 10418168C/A
4-4: 10431573T/C
4-4: 10436610A/C
4-4: 10476965T/C
4-4: 10512507A/G
4-4: 10549875C/T
4-4: 10644393A/G
4-4: 10646594G/T
4-4: 10688101C/A
4-4: 10724067C/A
4-4: 10771167T/C
4-4: 10771525T/C
4-4: 10782359G/T
4-4: 10787470T/C
4-4: 10801648T/C
4-4: 10810135A/G
4-4: 10864845C/T
4-4: 10877634C/T
4-4: 10879371G/C
4-4: 10907809A/T
4-4: 10946172T/C
4-4: 10993142A/G
4-4: 11017739C/T
4-4: 11032885C/T
4-4: 11064241G/A
4-4: 11139723T/C
4-4: 11198475T/C
4-4: 11198545G/A
4-4: 11198623C/T
4-4: 11198635C/G
4-4: 11198663G/A
4-4: 11198791C/T
4-4: 11198800C/T
4-4: 11198884G/C
4-4: 11198902C/T
4-4: 11198963G/T
4-4: 11215681A/G
4-4: 11241931A/T
4-4: 11286184A/T
4-4: 11303622A/T
4-4: 11374672C/A
4-4: 11548545G/A
4-4: 11557176G/A
4-4: 11557693T/C
4-4: 11592617A/G
4-4: 11630458C/T
4-4: 11644143A/G
4-4: 11651508C/A
4-4: 11669772C/T
4-4: 11671754A/C
4-4: 11673394T/A
4-4: 11696503A/G
4-4: 11711566T/C
4-4: 11722718T/C
4-4: 11756621G/A
4-4: 11771236A/G
4-4: 11800739T/A
4-4: 11887545G/A
4-4: 11918085T/C
4-4: 12048022T/C
4-4: 12048841C/A
4-4: 12049085T/C
4-4: 12049431A/T
4-4: 12053314A/G
4-4: 12053405G/A
4-4: 12055106G/C
4-4: 12055727C/T
4-4: 12056295A/G
4-4: 12057221G/A
4-4: 12061348C/T
4-4: 12071761T/C
4-4: 12122391C/T
4-4: 12134309A/G
4-4: 12187995G/A
4-4: 12237597T/G
4-4: 12276572A/T
4-4: 12342406G/T
4-4: 12381567T/C
4-4: 12439812C/T
4-4: 12515295A/T
4-4: 12543817T/A
4-4: 12593810A/G
4-4: 12698213C/T
4-4: 12772254G/A
4-4: 12791451T/C
4-4: 12824350T/C
4-4: 12854828G/T
4-4: 12886239G/T
4-4: 12937023C/A
4-4: 12970684G/A
4-4: 12991052G/A
4-4: 13015538A/G
4-4: 13025621A/G
4-4: 13049032G/T
4-4: 13057559A/C
4-4: 13058940A/G
4-4: 13059268C/A
4-4: 13059628G/A
4-4: 13100878T/C
4-4: 13105971T/C
4-4: 13105973T/C
4-4: 13150629G/A
4-4: 13166132G/A
4-4: 13256189A/C
4-4: 13325086G/A
4-4: 13430089G/C
4-4: 13463418C/T
4-4: 13488273G/A
4-4: 13540092C/G
4-4: 13618548A/G
4-4: 13645630G/C
4-4: 13675611G/T
4-4: 13747498G/A
4-4: 13861672C/G
4-4: 13879759T/C
4-4: 13934325T/C
4-4: 13977275A/T
4-4: 14002835T/C
4-4: 14016339G/C
4-4: 14097889A/T
4-4: 14112052C/A
4-4: 14215386C/T
4-4: 14237067A/T
4-4: 14237072T/A
4-4: 14307357C/G
4-4: 14375810A/G
4-4: 14401557C/G
4-4: 14436556T/C
4-4: 14447058G/A
4-4: 14491701G/A
4-4: 14569694C/T
4-4: 14596099G/T
4-4: 14616355C/T
4-4: 14658400T/C
4-4: 14776637A/C
4-4: 14792024T/C
4-4: 14804012A/C
4-4: 14942466C/T
4-4: 14968515C/A
4-4: 15002803G/A
4-4: 15089333C/A
4-4: 15110161T/A
4-4: 15246292A/G
4-4: 15365258A/C
4-4: 15399142C/A
4-4: 15434117A/G
4-4: 15463390G/A
4-4: 15486382A/G
4-4: 15519386C/T
4-4: 15586079C/G
4-4: 15658935C/T
4-4: 15669567G/T
4-4: 15730834A/G
4-4: 15766995G/A
4-4: 15778171A/G
4-4: 15836909C/A
4-4: 15882284C/A
4-4: 15942615T/A
4-4: 15961425C/A
4-4: 16097404T/C
4-4: 16130139A/G
4-4: 16200776T/A
4-4: 16230640T/G
4-4: 16258764T/A
4-4: 16288020G/A
4-4: 16302870T/C
4-4: 16400421C/G
4-4: 16470633T/C
4-4: 16498077A/G
4-4: 16571976G/A
4-4: 16683206A/G
4-4: 16710847C/G
4-4: 16732046C/A
4-4: 16855348C/A
4-4: 16900209G/T
4-4: 16964459G/T
4-4: 17191638A/C
4-4: 17196013T/C
4-4: 17270112T/G
4-4: 17308676C/T
4-4: 17481106A/G
4-4: 17566926T/A
4-4: 17655632T/C
4-4: 17717438C/T
4-4: 17766884A/G
4-4: 17785591T/C
4-4: 17814584A/T
4-4: 17994236A/T
4-4: 18209727A/T
4-4: 18227901C/T
4-4: 18229531T/A
4-4: 18255384T/G
4-4: 18267475A/T
4-4: 18461351G/T
4-4: 18512940C/T
4-4: 18624087G/A
4-4: 18651760G/C
4-4: 18653691A/G
4-4: 18710181A/G
4-4: 18710839G/A
4-4: 18712000A/G
4-4: 18712286C/T
4-4: 18725197A/G
4-4: 18814517T/C
4-4: 18818504C/T
4-4: 18830871T/C
4-4: 18841307A/T
4-4: 18946652T/A
4-4: 18971300T/C
4-4: 18992116A/G
4-4: 19041204C/T
4-4: 19071848T/A
4-4: 19123084G/A
4-4: 19125066A/G
4-4: 19142798G/T
4-4: 19155736T/C
4-4: 19178023G/A
4-4: 19218473A/T
4-4: 19240007T/C
4-4: 19261040A/T
4-4: 19262389C/T
4-4: 19277152G/T
4-4: 19297163A/G
4-4: 19312044C/A
4-4: 19333742G/A
4-4: 19344296G/A
4-4: 19405376T/C
4-4: 19448235T/C
4-4: 19467276C/A
4-4: 19468281A/T
4-4: 19496395G/A
4-4: 19554102A/G
4-4: 19642026A/G
4-4: 19668132T/C
4-4: 19776876C/A
4-4: 19813403A/G
4-4: 19902487A/T
4-4: 19957843A/T
4-4: 20006448T/G
4-4: 20095170G/A
4-4: 20106924G/A
4-4: 20118951T/G
4-4: 20181261C/T
4-4: 20218217C/T
4-4: 20222188T/C
4-4: 20247230G/A
4-4: 20276196C/T
4-4: 20302885A/G
4-4: 20376255C/G
4-4: 20449326A/T
4-4: 20463825G/A
4-4: 20615951A/G
4-4: 20626180T/C
4-4: 20697027C/A
4-4: 20814360T/G
4-4: 20862405A/T
4-4: 20896383A/G
4-4: 21002977C/T
4-4: 21034389G/C
4-4: 21069452C/T
4-4: 21101706A/T
4-4: 21113916C/T
4-4: 21319973A/G
4-4: 21488329G/T
4-4: 21514829G/A
4-4: 21558356G/A
4-4: 21574103A/C
4-4: 21667359C/A
4-4: 21679672T/A
4-4: 21709367T/G
4-4: 21736568C/A
4-4: 21800338G/A
4-4: 21823054T/C
4-4: 21838045C/T
4-4: 21856109G/A
4-4: 21954535A/G
4-4: 21965340C/G
4-4: 22177491G/A
4-4: 22235108G/A
4-4: 22275640G/T
4-4: 22287929A/C
4-4: 22305396A/T
4-4: 22323688G/A
4-4: 22382919T/A
4-4: 22427076C/A
4-4: 22454442G/A
4-4: 22717370T/C
4-4: 22765871A/G
4-4: 22779101A/T
4-4: 22937324A/G
4-4: 23085581G/A
4-4: 23103504T/G
4-4: 23138183A/G
4-4: 23163384A/T
4-4: 23190201C/A
4-4: 23267004G/A
4-4: 23455139C/T
4-4: 23545958G/A
4-4: 23576678C/T
4-4: 23699110T/G
4-4: 23771359T/A
4-4: 23798239A/T
4-4: 23864192C/T
4-4: 23866872T/A
4-4: 23867136G/A
4-4: 23867298A/G
4-4: 23867321G/A
4-4: 23867328A/C
4-4: 24106859A/C
4-4: 24345721G/T
4-4: 24345731G/T
4-4: 24345824T/G
4-4: 24345827G/T
4-4: 24345857T/G
4-4: 24345884C/T
4-4: 24345889C/T
4-4: 24345892T/G
4-4: 24345899A/G
4-4: 24345946T/A
4-4: 24345979T/C
4-4: 24345986A/G
4-4: 24345990T/C
4-4: 24346023G/A
4-4: 24346461C/T
4-4: 24346483T/C
4-4: 24346644G/A
4-4: 24346657C/T
4-4: 24346674C/T
4-4: 24346676A/G
4-4: 24346698A/G
4-4: 24346705T/C
4-4: 24620192T/G
4-4: 24633278T/C
4-4: 24710468T/C
4-4: 24778531A/C
4-4: 24789654T/C
4-4: 24866927G/T
4-4: 24910202C/T
4-4: 24923085A/G
4-4: 24930457T/C
4-4: 24936699G/A
4-4: 25035941T/C
4-4: 25053556G/T
4-4: 25068418G/A
4-4: 25082761C/T
4-4: 25118717G/A
4-4: 25321951G/A
4-4: 25425955G/A
4-4: 25438901A/T
4-4: 25459936G/A
4-4: 25598970A/G
4-4: 25692767G/A
4-4: 25695817G/A
4-4: 25708262T/A
4-4: 25709111A/G
4-4: 25711124A/G
4-4: 25737339G/A
4-4: 25795632T/C
4-4: 25825439C/A
4-4: 25942543T/C
4-4: 25984278C/T
4-4: 26090781C/T
4-4: 26198273C/T
4-4: 26285678T/C
4-4: 26440954G/A
4-4: 26441321A/T
4-4: 26441345A/C
4-4: 26441351T/C
4-4: 26441354T/A
4-4: 26441372C/T
4-4: 26441378T/A
4-4: 26441417C/T
4-4: 26441429T/C
4-4: 26441452T/A
4-4: 26632676C/G
4-4: 26782974G/A
4-4: 26795979T/C
4-4: 26810117G/A
4-4: 26825283A/G
4-4: 26831812G/A
4-4: 26834896A/T
4-4: 26836314A/G
4-4: 26836579T/C
4-4: 26836585A/G
4-4: 26837677C/T
4-4: 26837780T/C
4-4: 26837907G/A
4-4: 26837918G/T
4-4: 26837977G/T
4-4: 26838041T/C
4-4: 26838108A/G
4-4: 27030687C/T
4-4: 27050095T/A
4-4: 27061001C/A
4-4: 27077193C/A
4-4: 27113904C/T
4-4: 27150927A/T
4-4: 27161656C/T
4-4: 27173373T/C
4-4: 27257720A/G
4-4: 27313165A/T
4-4: 27409723T/A
4-4: 27417316G/A
4-4: 27436446G/C
4-4: 27453327C/T
4-4: 27491454A/C
4-4: 27501714G/A
4-4: 27518527T/C
4-4: 27550068C/G
4-4: 27557148T/C
4-4: 27573343G/T
4-4: 27647820C/A
4-4: 27772297A/G
4-4: 27782097C/T
4-4: 27787288A/C
4-4: 27973080T/C
4-4: 28029560C/T
4-4: 28136778C/T
4-4: 28153789G/A
4-4: 28214129T/C
4-4: 28254599T/G
4-4: 28285141G/A
4-4: 28333432T/C
4-4: 28333455C/T
4-4: 28333469C/T
4-4: 28333475T/C
4-4: 28333497T/A
4-4: 28333501G/A
4-4: 28333765A/G
4-4: 28333768T/C
4-4: 28584602T/G
4-4: 28655582C/T
4-4: 28667540T/C
4-4: 28761033A/G
4-4: 29045589T/C
4-4: 29114858T/C
4-4: 29140101G/A
4-4: 29172398G/A
4-4: 29204773G/A
4-4: 29296791T/C
4-4: 29333849C/T
4-4: 29380882G/T
4-4: 29440733G/A
4-4: 29451732T/C
4-4: 29470755G/A
4-4: 29498498A/G
4-4: 29520416T/A
4-4: 29547662G/A
4-4: 29603040G/A
4-4: 29630605C/T
4-4: 29700140A/T
4-4: 29700321G/A
4-4: 29719172A/C
4-4: 29785821T/C
4-4: 29817936T/A
4-4: 29847979C/T
4-4: 29936502A/G
4-4: 29957392T/C
4-4: 29976805G/A
4-4: 29992944A/T
4-4: 30034301C/T
4-4: 30264837C/T
4-4: 30403077T/C
4-4: 30489608T/A
4-4: 30556981T/A
4-4: 30571156G/A
4-4: 30807362G/A
4-4: 30920860C/A
4-4: 30937754T/C
4-4: 30963033A/T
4-4: 30984822G/A
4-4: 31003552A/G
4-4: 31397087T/G
4-4: 31440756A/T
4-4: 31441011A/G
4-4: 31553162C/T
4-4: 31572304T/C
4-4: 31585763C/A
4-4: 31604902T/A
4-4: 31621473T/C
4-4: 31636942C/G
4-4: 31649460C/T
4-4: 31676325A/T
4-4: 31710407G/A
4-4: 31723200A/C
4-4: 31771678C/T
4-4: 31796737A/C
4-4: 31834571G/A
4-4: 31919498G/A
4-4: 31995694G/C
4-4: 32048490T/A
4-4: 32104733C/T
4-4: 32191064A/G
4-4: 32302871C/T
4-4: 32383877G/A
4-4: 32416636G/A
4-4: 32449981G/A
4-4: 32460126C/T
4-4: 32480213T/G
4-4: 32531562T/C
4-4: 32587050T/C
4-4: 32624932A/T
4-4: 32636228T/C
4-4: 32652487C/A
4-4: 32720484T/A
4-4: 32734025A/G
4-4: 32755369T/C
4-4: 32757488G/A
4-4: 32771268G/A
4-4: 32877154T/C
4-4: 32881745C/T
4-4: 32906351A/T
4-4: 32987857G/A
4-4: 33035174T/C
4-4: 33174272G/A
4-4: 33300846C/T
4-4: 33446941A/G
4-4: 33449002G/A
4-4: 33514907T/C
4-4: 33567634T/C
4-4: 33691566T/G
4-4: 33701706T/A
4-4: 33767686T/A
4-4: 33825653G/C
4-4: 33842250G/A
4-4: 33852750G/A
4-4: 33867901A/G
4-4: 33904763G/A
4-4: 33955450C/T
4-4: 34022474A/G
4-4: 34102736T/C
4-4: 34131240G/A
4-4: 34131972A/G
4-4: 34190601G/T
4-4: 34214034G/A
4-4: 34290601C/T
4-4: 34322983C/T
4-4: 34340728T/C
4-4: 34368745A/G
4-4: 34407139C/T
4-4: 34407639A/C
4-4: 34425939A/G
4-4: 34475465T/C
4-4: 34490745G/C
4-4: 34511458C/A
4-4: 34616576G/A
4-4: 34626698T/C
4-4: 34682542T/C
4-4: 34699585C/A
4-4: 34721953C/A
4-4: 34731851T/A
4-4: 34781692T/G
4-4: 34837560A/C
4-4: 34947885G/A
4-4: 35120058A/G
4-4: 35130682A/G
4-4: 35172427T/A
4-4: 35203124G/A
4-4: 35219623G/A
4-4: 35231685A/T
4-4: 35246793C/T
4-4: 35308241A/C
4-4: 35348284T/C
4-4: 35441987T/C
4-4: 35518272A/C
4-4: 35594812G/T
4-4: 35646714C/T
4-4: 35664213G/A
4-4: 35675234T/A
4-4: 35692997C/T
4-4: 35694030G/A
4-4: 35697148T/C
4-4: 35707192A/G
4-4: 35752068G/C
4-4: 35752282G/A
4-4: 35767082A/T
4-4: 35859969G/A
4-4: 35897748A/G
4-4: 35990538T/C
4-4: 36036907G/A
4-4: 36108139G/A
4-4: 36135179C/A
4-4: 36189556G/A
4-4: 36245948T/C
4-4: 36272773G/A
4-4: 36334377A/G
4-4: 36382432A/T
4-4: 36449552C/T
4-4: 36475734G/T
4-4: 36535302G/C
4-4: 36564034A/T
4-4: 36652562G/A
4-4: 36769073T/C
4-4: 36771894A/G
4-4: 36808835A/G
4-4: 36836641G/T
4-4: 36880544G/A
4-4: 36911765A/G
4-4: 36961295C/T
4-4: 36975639G/A
4-4: 36999205T/C
4-4: 37004023C/T
4-4: 37007413C/T
4-4: 37056440T/G
4-4: 37170003T/C
4-4: 37266286C/T
4-4: 37267159T/A
4-4: 37322307G/A
4-4: 37336274G/A
4-4: 37388545T/A
4-4: 37473924G/T
4-4: 37497100A/C
4-4: 37521942T/G
4-4: 37573580C/G
4-4: 37583613A/G
4-4: 37597189G/T
4-4: 37607341G/A
4-4: 37617820G/A
4-4: 37633885T/A
4-4: 37639871A/G
4-4: 37640298T/C
4-4: 37640515G/A
4-4: 37661066G/T
4-4: 37700815T/A
4-4: 37737491T/C
4-4: 37739465A/G
4-4: 37739473T/C
4-4: 37739504A/G
4-4: 37739515C/T
4-4: 37916195C/T
4-4: 37934177A/G
4-4: 37958858T/C
4-4: 38031285G/T
4-4: 38122538G/A
4-4: 38171279C/T
4-4: 38187169C/T
4-4: 38309159G/A
4-4: 38338519A/C
4-4: 38366776T/C
4-4: 38368335C/T
4-4: 38389980T/G
4-4: 38404837A/G
4-4: 38406834A/G
4-4: 38467510A/G
4-4: 38618970A/G
4-4: 38674583G/T
4-4: 38799872G/A
4-4: 38850190T/C
4-4: 38952084G/T
4-4: 38975750A/C
4-4: 38976181G/C
4-4: 38994122A/C
4-4: 39041975G/A
4-4: 39073482A/T
4-4: 39075778A/T
4-4: 39086660C/T
4-4: 39091900T/.
4-4: 39107412C/T
4-4: 39229987G/A
4-4: 39231949A/G
4-4: 39285084G/A
4-4: 39342627G/A
4-4: 39403294C/T
4-4: 39465400G/A
4-4: 39494362C/A
4-4: 39586515C/T
4-4: 39613898T/A
4-4: 39677827G/A
4-4: 39766786C/T
4-4: 39767134G/A
4-4: 39808085A/T
4-4: 39818357G/A
4-4: 39830901A/T
4-4: 39831124G/A
4-4: 39831559G/A
4-4: 39832147A/G
4-4: 39832176G/A
4-4: 39832710T/C
4-4: 39834019G/A
4-4: 39843359G/A
4-4: 39844618T/G
4-4: 39846543A/G
4-4: 39887082A/T
4-4: 39900467G/A
4-4: 39953708A/G
4-4: 40106234G/C
4-4: 40162252T/C
4-4: 40219057A/G
4-4: 40327519T/C
4-4: 40392925C/A
4-4: 40478866A/G
4-4: 40538411C/A
4-4: 40587244G/A
4-4: 40656995A/G
4-4: 40667335C/G
4-4: 40667646A/G
4-4: 40724330C/A
4-4: 40736294C/A
4-4: 40810049T/A
4-4: 40844882T/C
4-4: 40937506C/A
4-4: 40977067G/A
4-4: 40999847C/A
4-4: 41040117C/T
4-4: 41043188A/C
4-4: 41106993C/T
4-4: 41119117C/A
4-4: 41266555C/T
4-4: 41272830A/G
4-4: 41306009G/A
4-4: 41316305T/C
4-4: 41338158G/C
4-4: 41368669C/T
4-4: 41460549A/G
4-4: 41533195T/C
4-4: 41560281C/T
4-4: 41587489T/A
4-4: 41655185A/T
4-4: 41673389G/C
4-4: 41685537G/A
4-4: 41730958G/T
4-4: 41736463G/C
4-4: 41737005T/G
4-4: 41745467T/C
4-4: 41776420A/G
4-4: 41795898C/G
4-4: 41878720G/C
4-4: 41895032G/T
4-4: 41911630G/C
4-4: 42032007C/T
4-4: 42075285C/T
4-4: 42097876G/A
4-4: 42190782C/T
4-4: 42292683T/A
4-4: 42362181T/C
4-4: 42365852A/G
4-4: 42401450T/G
4-4: 42462372C/A
4-4: 42488289T/G
4-4: 42489016G/A
4-4: 42501001A/G
4-4: 42526210G/T
4-4: 42569359C/A
4-4: 42615448C/A
4-4: 42639531G/A
4-4: 42655404A/T
4-4: 42667289A/G
4-4: 42674872T/C
4-4: 42693831C/T
4-4: 42722306C/T
4-4: 42741626C/T
4-4: 42765955T/C
4-4: 42777121A/G
4-4: 42802287A/C
4-4: 42835914A/G
4-4: 42900674C/T
4-4: 42933541A/C
4-4: 42944333C/T
4-4: 42951261A/G
4-4: 42953684C/T
4-4: 42953693C/T
4-4: 42964818C/T
4-4: 42982606A/G
4-4: 43034811C/T
4-4: 43047904T/C
4-4: 43050634T/C
4-4: 43052613G/A
4-4: 43064019C/T
4-4: 43065135C/T
4-4: 43065778T/A
4-4: 43067369T/A
4-4: 43080467T/C
4-4: 43091427A/G
4-4: 43117724G/C
4-4: 43186380C/T
4-4: 43188981A/G
4-4: 43199085G/A
4-4: 43269132C/T
4-4: 43283411A/G
4-4: 43324779A/G
4-4: 43358060C/T
4-4: 43375236C/T
4-4: 43453336A/C
4-4: 43557281A/G
4-4: 43558864G/A
4-4: 43559226G/A
4-4: 43597138A/G
4-4: 43690358T/C
4-4: 43767385A/G
4-4: 43827175G/A
4-4: 43847073C/T
4-4: 43867698G/A
4-4: 43882022C/A
4-4: 43915642A/G
4-4: 43978906T/C
4-4: 43995786G/A
4-4: 44038173A/T
4-4: 44063021T/G
4-4: 44075685T/C
4-4: 44082093T/C
4-4: 44113173A/C
4-4: 44122267T/A
4-4: 44126797A/T
4-4: 44296770C/T
4-4: 44302047G/A
4-4: 44331330G/A
4-4: 44384871A/G
4-4: 44560785C/G
4-4: 44567793A/G
4-4: 44599641G/A
4-4: 44633644A/G
4-4: 44682327G/A
4-4: 44724858T/G
4-4: 44786699T/C
4-4: 44820165T/A
4-4: 44844760C/T
4-4: 44937601A/G
4-4: 44990045G/A
4-4: 45019160A/G
4-4: 45111575C/T
4-4: 45180010T/C
4-4: 45229418T/C
4-4: 45261440G/A
4-4: 45320619C/T
4-4: 45426198C/T
4-4: 45490849C/G
4-4: 45535198T/A
4-4: 45613372G/A
4-4: 45658454C/T
4-4: 45674753C/A
4-4: 45719323T/G
4-4: 45746260C/A
4-4: 45746839A/G
4-4: 45747085C/T
4-4: 45774915C/A
4-4: 45801565C/T
4-4: 45879917T/G
4-4: 46016779A/T
4-4: 46054989C/T
4-4: 46173242G/C
4-4: 46227871A/G
4-4: 46246071G/A
4-4: 46397513A/G
4-4: 46473912G/T
4-4: 46517730C/T
4-4: 46532480A/G
4-4: 46635472A/T
4-4: 46649198C/T
4-4: 46667934T/C
4-4: 46697034T/C
4-4: 46752418T/C
4-4: 46795366T/C
4-4: 46823441C/A
4-4: 46843065T/C
4-4: 46844500C/T
4-4: 46880595T/C
4-4: 46892909A/C
4-4: 47012532C/T
4-4: 47115970C/G
4-4: 47165844T/C
4-4: 47181644A/G
4-4: 47210024A/C
4-4: 47215364G/A
4-4: 47278047A/G
4-4: 47317893C/T
4-4: 47357140C/T
4-4: 47399364A/C
4-4: 47438277T/C
4-4: 47492316C/T
4-4: 47492543G/A
4-4: 47579736A/C
4-4: 47606227C/T
4-4: 47640438G/A
4-4: 47678070C/T
4-4: 47693287G/A
4-4: 47738449T/A
4-4: 47754859G/A
4-4: 47783765A/G
4-4: 47788309A/C
4-4: 47822824T/A
4-4: 47862101C/T
4-4: 47897488G/C
4-4: 47967326A/T
4-4: 48015799T/C
4-4: 48021537T/C
4-4: 48049287T/C
4-4: 48136095C/T
4-4: 48183865T/C
4-4: 48256015A/T
4-4: 48304106C/T
4-4: 48344042T/A
4-4: 48371614A/G
4-4: 48397679C/G
4-4: 48423339C/A
4-4: 48455954G/A
4-4: 48506195T/C
4-4: 48511041T/G
4-4: 48521506A/T
4-4: 48547581A/G
4-4: 48654998C/T
4-4: 48702143G/A
4-4: 48859844A/C
4-4: 48938604G/C
4-4: 49066620G/A
4-4: 49081374T/C
4-4: 49081392A/G
4-4: 49213451A/C
4-4: 49296104C/A
4-4: 49313402A/T
4-4: 49346080C/A
4-4: 49375913T/A
4-4: 49407913C/A
4-4: 49444002C/T
4-4: 49454471T/C
4-4: 49530980A/G
4-4: 49581800A/G
4-4: 49627350C/A
4-4: 49687648A/G
4-4: 49804072T/C
4-4: 49871065G/T
4-4: 49890016G/A
4-4: 49891722A/G
4-4: 49954195A/T
4-4: 49997244A/T
4-4: 50041631C/A
4-4: 50052610T/C
4-4: 50094580A/T
4-4: 50130755A/G
4-4: 50172716A/T
4-4: 50266175T/G
4-4: 50291481G/T
4-4: 50318027A/G
4-4: 50413270A/T
4-4: 50419464A/C
4-4: 50423534T/A
4-4: 50439976T/C
4-4: 50523007T/C
4-4: 50650501T/C
4-4: 50781829C/T
4-4: 50781889A/T
4-4: 50782159T/C
4-4: 50782230G/C
4-4: 51024512A/G
4-4: 51079672G/T
4-4: 51162122A/G
4-4: 51192047G/A
4-4: 51235156A/G
4-4: 51247160T/G
4-4: 51361450A/T
4-4: 51371534C/A
4-4: 51402017G/A
4-4: 51461959A/G
4-4: 51481255G/A
4-4: 51491627C/T
4-4: 51492683A/G
4-4: 51507257A/C
4-4: 51533718A/C
4-4: 51625561C/A
4-4: 51649043C/G
4-4: 51811117C/T
4-4: 51821231A/G
4-4: 51840158T/C
4-4: 51853303T/G
4-4: 51888659C/T
4-4: 51932244A/G
4-4: 51983538T/A
4-4: 51988952T/A
4-4: 51988984G/A
4-4: 52034098A/G
4-4: 52083941G/A
4-4: 52096135G/A
4-4: 52111597A/T
4-4: 52139655T/G
4-4: 52174468G/A
4-4: 52236870A/G
4-4: 52266031C/T
4-4: 52303588A/C
4-4: 52332734A/G
4-4: 52397964C/T
4-4: 52406020T/C
4-4: 52408933A/G
4-4: 52424509C/T
4-4: 52461262A/G
4-4: 52471390G/A
4-4: 52486026T/C
4-4: 52488807G/A
4-4: 52489179G/A
4-4: 52499221T/C
4-4: 52548278G/A
4-4: 52637562C/T
4-4: 52701720C/A
4-4: 52896902A/G
4-4: 52910486T/G
4-4: 52926012T/C
4-4: 52932854T/C
4-4: 53027183T/G
4-4: 53275566T/C
4-4: 53355203G/A
4-4: 53441530G/A
4-4: 53574471T/C
4-4: 53654732T/G
4-4: 53656000C/G
4-4: 53670660C/G
4-4: 53683500C/T
4-4: 53721509C/T
4-4: 53751323G/A
4-4: 53806261G/A
4-4: 53878235T/G
4-4: 53927735C/T
4-4: 53953692G/C
4-4: 53971221T/G
4-4: 54288555T/C
4-4: 54296222A/G
4-4: 54550242C/T
4-4: 54574012T/C
4-4: 54584984T/A
4-4: 54608234A/T
4-4: 54663249T/C
4-4: 54983186A/G
4-4: 55046830T/A
4-4: 55161104C/T
4-4: 55176305G/C
4-4: 55193683A/T
4-4: 55219158A/C
4-4: 55236917T/G
4-4: 55296347G/A
4-4: 55313161T/C
4-4: 55638943C/T
4-4: 55711098T/C
4-4: 55884567C/T
4-4: 56029284T/C
4-4: 56216679G/T
4-4: 56228988C/G
4-4: 56415157A/T
4-4: 56552617A/G
4-4: 56757948A/T
4-4: 56828032A/G
4-4: 57007852T/C
4-4: 57072019G/A
4-4: 57093976C/G
4-4: 57306526T/A
4-4: 57401404G/A
4-4: 57411604A/G
4-4: 57438676A/C
4-4: 57459525C/T
4-4: 57503830G/A
4-4: 57538639G/T
4-4: 57614586C/T
4-4: 57713109C/T
4-4: 57772262C/T
4-4: 57873746C/T
4-4: 57882919G/A
4-4: 57941481C/T
4-4: 57998346G/A
4-4: 58051086T/C
4-4: 58130991C/T
4-4: 58189894A/G
4-4: 58217240C/T
4-4: 58263367G/A
4-4: 58296332T/C
4-4: 58335653G/T
4-4: 58402116A/C
4-4: 58417384G/A
4-4: 58446954C/T
4-4: 58473189A/G
4-4: 58500155A/T
4-4: 58609584C/T
4-4: 58755157A/T
4-4: 58833353T/C
4-4: 58856552G/C
4-4: 58900688C/G
4-4: 58996541C/T
4-4: 59023836C/A
4-4: 59087647T/A
4-4: 59204791T/C
4-4: 59228765G/A
4-4: 59230993T/C
4-4: 59256347G/C
4-4: 59288645T/A
4-4: 59348942G/A
4-4: 59362424A/G
4-4: 59388440G/A
4-4: 59439453G/T
4-4: 59473163C/T
4-4: 59700415C/T
4-4: 59704470T/C
4-4: 59733333G/A
4-4: 59785538T/C
4-4: 59815020C/T
4-4: 59954756G/C
4-4: 59954790C/T
4-4: 59957857C/T
4-4: 60014426T/C
4-4: 60057636G/A
4-4: 60086986A/C
4-4: 60129263A/G
4-4: 60156314G/A
4-4: 60181929G/A
4-4: 60218896A/T
4-4: 60249325C/G
4-4: 60344095G/A
4-4: 60352299A/G
4-4: 60390870T/A
4-4: 60425134G/A
4-4: 60488808C/T
4-4: 60531771C/T
4-4: 60550487G/A
4-4: 60557337G/.
4-4: 60564237C/T
4-4: 60565293C/T
4-4: 60565383G/A
4-4: 60649373G/T
4-4: 60667957G/A
4-4: 60700074C/T
4-4: 60739505G/A
4-4: 60770862G/A
4-4: 60800600G/C
4-4: 60840074T/C
4-4: 60917484G/A
4-4: 60967315T/C
4-4: 60977390C/T
4-4: 61048415A/T
4-4: 61197158T/A
4-4: 61306598T/C
4-4: 61354910A/C
4-4: 61418679A/G
4-4: 61462207G/T
4-4: 61566905A/T
4-4: 61579476C/A
4-4: 61593256G/A
4-4: 61606369C/G
4-4: 61659880C/A
4-4: 61761475T/C
4-4: 61764176G/A
4-4: 61779140A/C
4-4: 61795635T/C
4-4: 61845972G/T
4-4: 61991151T/C
4-4: 61991675T/C
4-4: 61992249A/G
4-4: 62049369A/G
4-4: 62088992T/A
4-4: 62130726G/A
4-4: 62242659A/T
4-4: 62267895C/G
4-4: 62293707T/C
4-4: 62313379T/C
4-4: 62364682C/T
4-4: 62466605C/T
4-4: 62519651A/G
4-4: 62535438A/G
4-4: 62563121C/T
4-4: 62622057C/T
4-4: 62623136T/A
4-4: 62623155A/G
4-4: 62689035C/T
4-4: 62708804T/C
4-4: 62758593G/T
4-4: 62789093T/G
4-4: 62837239T/C
4-4: 62865028A/T
4-4: 62872691G/A
4-4: 62888849A/T
4-4: 62907576G/A
4-4: 62909581A/G
4-4: 62950351G/A
4-4: 62982746G/T
4-4: 63031619A/T
4-4: 63152164C/T
4-4: 63152354C/T
4-4: 63174978T/C
4-4: 63175738C/A
4-4: 63215441A/C
4-4: 63255857G/C
4-4: 63282503T/A
4-4: 63412205A/T
4-4: 63421074T/C
4-4: 63447151T/G
4-4: 63522151T/C
4-4: 63578251G/A
4-4: 63681513A/G
4-4: 63684737A/T
4-4: 63741256A/C
4-4: 63784281G/A
4-4: 63816514G/C
4-4: 63868049G/A
4-4: 63926223T/G
4-4: 63960569T/C
4-4: 64002328C/A
4-4: 64033991G/A
4-4: 64060178T/C
4-4: 64084160A/G
4-4: 64097274T/C
4-4: 64136993C/G
4-4: 64236844G/A
4-4: 64284952C/A
4-4: 64321140A/T
4-4: 64387217A/G
4-4: 64487717G/T
4-4: 64494941A/T
4-4: 64496021C/A
4-4: 64589238A/G
4-4: 64615753A/G
4-4: 64692263T/C
4-4: 64735080G/C
4-4: 64796727C/A
4-4: 64839397T/C
4-4: 64849607G/A
4-4: 64863052A/T
4-4: 64892389A/T
4-4: 64932498C/T
4-4: 64954298G/A
4-4: 64966369G/A
4-4: 64994309C/T
4-4: 65033205T/A
4-4: 65083727A/G
4-4: 65109011T/C
4-4: 65135653C/T
4-4: 65178154G/A
4-4: 65278043A/G
4-4: 65296958A/G
4-4: 65341881T/C
4-4: 65414988T/C
4-4: 65468325C/T
4-4: 65573104A/C
4-4: 65604468T/C
4-4: 65634252A/G
4-4: 65686411T/A
4-4: 65744497C/T
4-4: 65786717T/A
4-4: 65845702T/G
4-4: 65859267T/C
4-4: 65890136G/A
4-4: 65910940G/A
4-4: 65959592C/T
4-4: 65973138A/G
4-4: 65987982G/A
4-4: 66043729G/A
4-4: 66086702T/C
4-4: 66112097T/A
4-4: 66124883G/A
4-4: 66242069G/A
4-4: 66254138C/T
4-4: 66259452C/A
4-4: 66279518G/C
4-4: 66368570T/C
4-4: 66415177T/C
4-4: 66534576A/G
4-4: 66539256G/A
4-4: 66560764T/C
4-4: 66572469G/C
4-4: 66625280T/A
4-4: 66644868T/C
4-4: 66691297T/A
4-4: 66808291G/C
4-4: 66829010C/T
4-4: 66865196T/G
4-4: 66895264T/G
4-4: 66930530T/G
4-4: 66960256C/G
4-4: 66992128C/T
4-4: 67016639C/T
4-4: 67042239A/C
4-4: 67073349G/A
4-4: 67156157T/C
4-4: 67204347A/G
4-4: 67205671T/A
4-4: 67207337C/T
4-4: 67207353T/A
4-4: 67247808C/T
4-4: 67278058T/C
4-4: 67334656T/A
4-4: 67355782C/A
4-4: 67424056A/T
4-4: 67456192A/G
4-4: 67489632T/C
4-4: 67503191T/C
4-4: 67515282G/A
4-4: 67529284A/G
4-4: 67627739T/C
4-4: 67642570C/T
4-4: 67655731G/C
4-4: 67719356T/C
4-4: 67734006G/A
4-4: 67758023T/A
4-4: 67764724C/T
4-4: 67773091C/A
4-4: 67783439A/T
4-4: 67794048C/T
4-4: 67796177C/T
4-4: 67798429C/T
4-4: 67809214C/T
4-4: 67841264A/G
4-4: 67852379A/G
4-4: 67865227C/T
4-4: 67869807G/C
4-4: 67871285A/G
4-4: 67872684A/G
4-4: 67878979T/A
4-4: 67889598C/T
4-4: 67916521T/A
4-4: 67949103T/G
4-4: 67988839C/T
4-4: 68020441T/G
4-4: 68062663A/G
4-4: 68095938T/G
4-4: 68138874T/C
4-4: 68252938T/G
4-4: 68288727C/T
4-4: 68302716C/T
4-4: 68328561A/T
4-4: 68347408C/T
4-4: 68369158G/T
4-4: 68388270C/G
4-4: 68411459G/A
4-4: 68454499T/C
4-4: 68480192A/G
4-4: 68482859G/A
4-4: 68488907A/G
4-4: 68491020C/G
4-4: 68512115T/A
4-4: 68524362G/A
4-4: 68612886T/C
4-4: 68779459A/T
4-4: 68784300G/A
4-4: 68795224T/C
4-4: 68934511C/A
4-4: 68956715C/G
4-4: 69061508T/C
4-4: 69098020C/G
4-4: 69132026A/T
4-4: 69167167G/A
4-4: 69248655C/T
4-4: 69283822G/T
4-4: 69329872T/C
4-4: 69371976A/C
4-4: 69485558T/G
4-4: 69549325A/G
4-4: 69563956A/C
4-4: 69610313A/T
4-4: 69612601C/T
4-4: 69682109C/T
4-4: 69718093A/G
4-4: 69749158T/C
4-4: 69799212A/T
4-4: 69828517G/A
4-4: 69876789T/C
4-4: 69893360C/T
4-4: 69906907T/A
4-4: 69923459C/T
4-4: 69938109G/A
4-4: 69948984A/G
4-4: 69968186A/G
4-4: 69992719A/G
4-4: 70029723T/G
4-4: 70042001A/G
4-4: 70060988G/A
4-4: 70110801T/G
4-4: 70132112A/G
4-4: 70151520A/C
4-4: 70193525G/A
4-4: 70308159A/T
4-4: 70344276T/G
4-4: 70394321A/G
4-4: 70420181G/T
4-4: 70457078C/T
4-4: 70508685C/A
4-4: 70535395C/T
4-4: 70593294G/A
4-4: 70618932C/T
4-4: 70667465G/A
4-4: 70785340T/C
4-4: 70794178A/G
4-4: 70965793T/A
4-4: 70991375G/T
4-4: 71028359C/T
4-4: 71079470C/T
4-4: 71179320G/A
4-4: 71191564C/G
4-4: 71208611T/C
4-4: 71275975A/G
4-4: 71288411C/G
4-4: 71403740C/A
4-4: 71489046C/A
4-4: 71490245A/C
4-4: 71503813G/T
4-4: 71520441C/T
4-4: 71540300G/A
4-4: 71638680G/T
4-4: 71716863T/A
4-4: 71829889G/T
4-4: 71909955A/C
4-4: 71923398A/T
4-4: 72007288G/A
4-4: 72077250A/G
4-4: 72115388G/C
4-4: 72149878T/A
4-4: 72244009G/T
4-4: 72347298C/T
4-4: 72684580A/T
4-4: 72787347C/T
4-4: 72817015A/G
4-4: 72828532A/G
4-4: 73029198C/T
4-4: 73108178A/G
4-4: 73297738T/C
4-4: 73339019G/T
4-4: 73374328G/A
4-4: 73450356C/T
4-4: 73471432A/C
4-4: 73496228G/A
4-4: 73603653A/G
4-4: 73752607C/T
4-4: 74009577A/T
4-4: 74091145A/T
4-4: 74461795T/G
4-4: 74475327T/C
4-4: 74504672C/A
4-4: 74521660A/G
4-4: 74705925C/A
4-4: 74769742C/T
4-4: 74924925A/C
4-4: 74978966C/T
4-4: 74979004T/C
4-4: 74979034C/T
4-4: 74979047G/A
4-4: 74979838A/T
4-4: 74979894G/T
4-4: 74979912C/A
4-4: 74980012C/G
4-4: 74980021T/G
4-4: 74980023A/C
4-4: 74980043C/G
4-4: 74980124G/T
4-4: 74980406C/G
4-4: 75006248A/T
4-4: 75120629A/C
4-4: 75130176T/C
4-4: 75164402C/T
4-4: 75231789C/T
4-4: 75279035A/C
4-4: 75290554T/C
4-4: 75362409T/A
4-4: 75396845T/C
4-4: 75683265T/C
4-4: 75759357A/T
4-4: 75830091A/C
4-4: 75872108C/T
4-4: 75919410C/T
4-4: 76028319C/G
4-4: 76385479T/C
4-4: 76418365A/C
4-4: 76458445G/A
4-4: 76628441A/G
4-4: 76790240T/G
4-4: 76829752A/G
4-4: 76875184G/C
4-4: 76931045C/T
4-4: 76957981C/T
4-4: 76987641A/G
4-4: 77052096G/A
4-4: 77077375A/G
4-4: 77199101A/G
4-4: 77383940T/A
4-4: 77444183A/T
4-4: 77455010C/T
4-4: 77539266C/T
4-4: 77613347T/A
4-4: 77678447A/T
4-4: 77719699A/T
4-4: 77782655A/G
4-4: 77886090C/G
4-4: 77939005G/A
4-4: 77972512T/C
4-4: 78142388T/A
4-4: 78267045C/T
4-4: 78312410T/G
4-4: 78355751C/G
4-4: 78396326G/A
4-4: 78428533T/G
4-4: 78499505G/C
4-4: 78546424A/T
4-4: 78582112C/T
4-4: 78609721T/C
4-4: 78676719A/T
4-4: 78788302G/A
4-4: 78895691C/T
4-4: 78956226T/A
4-4: 79099501C/T
4-4: 79140415T/A
4-4: 79165940T/C
4-4: 79247866C/T
4-4: 79277418G/A
4-4: 79334672C/A
4-4: 79360436A/G
4-4: 79495977T/C
4-4: 79505612G/C
4-4: 79525209C/T
4-4: 79596650A/C
4-4: 79622644T/A
4-4: 79701524C/T
4-4: 79752122G/A
4-4: 79777156A/C
4-4: 79821063T/C
4-4: 79843415C/T
4-4: 79869513T/C
4-4: 79909552T/C
4-4: 79957934C/T
4-4: 80010239A/C
4-4: 80036505A/G
4-4: 80046529C/T
4-4: 80077662T/C
4-4: 80094350T/G
4-4: 80179987A/G
4-4: 80246081G/A
4-4: 80309897T/C
4-4: 80309909C/T
4-4: 80325115G/A
4-4: 80344222T/C
4-4: 80364049C/G
4-4: 80381722G/T
4-4: 80399785T/A
4-4: 80418382C/A
4-4: 80439782A/G
4-4: 80560255G/A
4-4: 80563830G/T
4-4: 80569835G/A
4-4: 80571779A/G
4-4: 80585776A/G
4-4: 80613715G/A
4-4: 80657562C/T
4-4: 80697376C/T
4-4: 80725251A/G
4-4: 80763239A/G
4-4: 80840656C/A
4-4: 80868445A/C
4-4: 80912621A/G
4-4: 80970047T/C
4-4: 80971651G/A
4-4: 81003433C/T
4-4: 81036633A/T
4-4: 81116348A/G
4-4: 81146443C/T
4-4: 81184737G/C
4-4: 81196939T/C
4-4: 81222793G/C
4-4: 81262205A/G
4-4: 81288052A/C
4-4: 81328886A/C
4-4: 81370892A/T
4-4: 81401698T/A
4-4: 81438741G/T
4-4: 81486119T/C
4-4: 81553778T/C
4-4: 81568068C/T
4-4: 81632966T/G
4-4: 81655143C/T
4-4: 81711774A/G
4-4: 81729872T/A
4-4: 81758314A/G
4-4: 81791057A/T
4-4: 81841002T/C
4-4: 81882368A/T
4-4: 81933918G/A
4-4: 82050603T/A
4-4: 82138462T/A
4-4: 82147104G/A
4-4: 82147115T/C
4-4: 82147120T/C
4-4: 82147130G/C
4-4: 82147156C/T
4-4: 82371013T/A
4-4: 82497535T/G
4-4: 82498854G/A
4-4: 82509888A/G
4-4: 82572853C/T
4-4: 82818879T/C
4-4: 82830690G/A
5-1: 124A/T
5-1: 409085T/C
5-1: 434920T/G
5-1: 477399A/G
5-1: 516951T/G
5-1: 896998A/T
5-1: 1109716T/C
5-1: 2097494A/T
5-1: 2106222T/A
5-1: 2226913T/C
5-1: 2260943C/T
5-1: 2333895T/A
5-1: 2369873G/A
5-1: 2460080G/A
5-1: 2709055A/C
5-1: 2756595A/G
5-1: 2794943C/T
5-1: 2829492A/G
5-1: 2906371G/T
5-1: 2968834T/C
5-1: 3002958G/A
5-1: 3061586G/A
5-1: 3109236G/C
5-1: 3142957A/G
5-1: 3155285A/C
5-1: 3267522C/A
5-1: 3277671G/A
5-1: 3298545C/T
5-1: 3369480C/T
5-1: 3544242C/T
5-1: 3552493G/A
5-1: 3687112C/T
5-1: 3749247A/G
5-1: 3777980T/C
5-1: 3782223G/T
5-1: 3788564G/T
5-1: 3790040C/T
5-1: 3897522T/C
5-1: 3944107G/A
5-1: 3995136A/G
5-1: 4092326T/G
5-1: 4205899C/T
5-1: 4240187C/T
5-1: 4248923A/G
5-1: 4331897G/A
5-1: 4434545A/C
5-1: 4459819A/G
5-1: 4490480G/A
5-1: 4530081C/T
5-1: 4555646G/T
5-1: 4587479T/A
5-1: 4631661A/G
5-1: 4646339A/G
5-1: 4735849G/A
5-1: 4785782C/A
5-1: 4844775G/A
5-1: 4891718C/T
5-1: 4993175C/T
5-1: 5034517G/A
5-1: 5149635C/A
5-1: 5253159T/G
5-1: 5309845C/T
5-1: 5398580A/G
5-1: 5450846C/T
5-1: 5547752T/C
5-1: 5579119T/C
5-1: 5618867C/T
5-1: 5624368G/C
5-1: 5624435G/A
5-1: 5707474A/C
5-1: 5720416T/C
5-1: 5932917C/G
5-1: 5944091C/.
5-1: 5944140T/C
5-1: 6073662C/A
5-1: 6092359A/T
5-1: 6104000C/A
5-1: 6105815T/G
5-1: 6178267G/T
5-1: 6193655A/G
5-1: 6284071T/C
5-1: 6337259C/T
5-1: 6444376T/G
5-1: 6465308C/G
5-1: 6552613T/A
5-1: 6830720A/G
5-1: 6852854C/T
5-1: 6868115C/T
5-1: 6878255A/G
5-1: 6889584A/G
5-1: 6905106C/G
5-1: 6908966C/T
5-1: 6919397T/G
5-1: 6980829T/A
5-1: 7052103G/A
5-1: 7058592G/T
5-1: 7059098C/T
5-1: 7059107G/A
5-1: 7060070C/T
5-1: 7101885C/T
5-1: 7121816C/G
5-1: 7144345C/T
5-1: 7159986A/C
5-1: 7170554T/A
5-1: 7207565C/A
5-1: 7241789A/T
5-1: 7280781G/T
5-1: 7302458A/T
5-1: 7402917A/G
5-1: 7444822C/T
5-1: 7565531G/A
5-1: 7683542A/G
5-1: 7761214C/A
5-1: 7813068T/C
5-1: 7923187C/A
5-1: 7994483G/T
5-1: 8136280G/A
5-1: 8141274A/G
5-1: 8237787A/T
5-1: 8292231G/A
5-1: 8319410T/C
5-1: 8320575G/A
5-1: 8324602A/T
5-1: 8370665C/T
5-1: 8419267G/C
5-1: 8455729A/C
5-1: 8487739T/C
5-1: 8530861A/T
5-1: 8563399T/C
5-1: 8618368C/T
5-1: 8655204A/G
5-1: 8717412G/A
5-1: 8821401A/T
5-1: 8881174G/C
5-1: 8922653C/T
5-1: 8952827A/G
5-1: 9045101G/A
5-1: 9163977A/G
5-1: 9198820T/C
5-1: 9357021C/G
5-1: 9503808G/A
5-1: 9505477G/A
5-1: 9525864G/A
5-1: 9555192G/A
5-1: 9600243G/A
5-1: 9729860C/A
5-1: 9793116A/T
5-1: 9831906G/A
5-1: 9859397T/G
5-1: 9886495C/G
5-1: 9897978T/C
5-1: 9933900C/T
5-1: 9976287A/G
5-1: 10007212C/A
5-1: 10042129G/A
5-1: 10068669G/A
5-1: 10129275G/A
5-1: 10163658G/T
5-1: 10246062A/C
5-1: 10321625T/C
5-1: 10358584G/A
5-1: 10387597C/T
5-1: 10417042A/C
5-1: 10442543A/G
5-1: 10488333G/C
5-1: 10498514G/C
5-1: 10520544C/T
5-1: 10592320A/T
5-1: 10657572A/T
5-1: 10704238T/C
5-1: 10748910T/C
5-1: 10818643T/C
5-1: 10844051T/C
5-1: 10901559G/A
5-1: 10944081C/T
5-1: 10956014T/C
5-1: 10982890T/G
5-1: 11042120A/G
5-1: 11107951C/A
5-1: 11132212T/A
5-1: 11168894T/G
5-1: 11180669A/G
5-1: 11384838A/T
5-1: 11478408A/G
5-1: 11568022A/G
5-1: 11667543G/A
5-1: 11779909A/G
5-1: 11843255A/G
5-1: 11858556T/A
5-1: 11915430C/A
5-1: 11942706G/T
5-1: 11970435C/T
5-1: 12071745T/C
5-1: 12104429G/A
5-1: 12116486T/C
5-1: 12127831A/G
5-1: 12147263C/T
5-1: 12159098G/T
5-1: 12213404C/T
5-1: 12252237A/G
5-1: 12296773G/A
5-1: 12378797C/T
5-1: 12469796A/T
5-1: 12513312T/C
5-1: 12555763C/T
5-1: 12566143T/C
5-1: 12626067C/T
5-1: 12765756T/C
5-1: 12767032T/C
5-1: 12790431T/C
5-1: 12815765T/C
5-1: 12956463G/A
5-1: 13066385G/A
5-1: 13137101G/A
5-1: 13203348C/T
5-1: 13211608A/G
5-1: 13287624C/T
5-1: 13312610G/T
5-1: 13323079C/T
5-1: 13335447C/T
5-1: 13349342C/A
5-1: 13380286T/C
5-1: 13389137T/G
5-1: 13389583G/A
5-1: 13421997T/A
5-1: 13437814A/C
5-1: 13967986T/A
5-1: 14139993G/A
5-1: 14160758G/A
5-1: 14277070C/T
5-1: 14362239A/G
5-1: 14663633G/A
5-1: 14718185A/T
5-1: 14758047A/G
5-1: 14761890A/G
5-1: 14761929A/T
5-1: 14762020T/C
5-1: 14762048G/A
5-1: 14762077G/A
5-1: 14762079G/A
5-1: 14762080C/T
5-1: 14762095A/G
5-1: 14762468G/A
5-1: 14762662A/T
5-1: 14763088A/G
5-1: 14769887C/T
5-1: 14802126A/T
5-1: 14803598G/A
5-1: 14803671A/G
5-1: 14817129G/A
5-1: 14842202C/T
5-1: 14901985T/A
5-1: 14971143T/C
5-1: 15072011T/C
5-1: 15124587T/C
5-1: 15125951T/C
5-1: 15147090A/C
5-1: 15150391G/A
5-1: 15180702A/G
5-1: 15225010C/A
5-1: 15246689C/T
5-1: 15270004C/T
5-1: 15352475C/A
5-1: 15545552C/T
5-1: 15580852G/A
5-1: 15597990C/T
5-1: 15635379A/T
5-1: 15652540A/C
5-1: 15772068C/T
5-1: 15789999A/G
5-1: 15945449G/C
5-1: 15976499G/T
5-1: 15990711G/A
5-1: 15992411C/T
5-1: 16047423C/A
5-1: 16152052G/A
5-1: 16187677T/C
5-1: 16285033A/G
5-1: 16300987T/G
5-1: 16305025G/A
5-1: 16321536C/G
5-1: 16351035G/T
5-1: 16370190T/A
5-1: 16386475G/A
5-1: 16413892G/A
5-1: 16444548T/A
5-1: 16520256A/G
5-1: 16543040A/G
5-1: 16579603G/A
5-1: 16588372C/A
5-1: 16638220G/C
5-1: 16679553C/A
5-1: 16800500T/C
5-1: 16802353C/A
5-1: 16859108G/A
5-1: 16919933G/T
5-1: 16942911T/A
5-1: 16978096A/T
5-1: 16989275T/A
5-1: 17024981A/T
5-1: 17079895G/A
5-1: 17120730C/G
5-1: 17257645C/A
5-1: 17269400C/T
5-1: 17282893A/G
5-1: 17301767G/A
5-1: 17334081G/T
5-1: 17349006G/A
5-1: 17360662A/T
5-1: 17394898G/C
5-1: 17447626C/T
5-1: 17476186G/A
5-1: 17547707G/T
5-1: 17625174T/A
5-1: 17659611A/G
5-1: 17693966T/G
5-1: 17838438G/A
5-1: 17839700A/C
5-1: 17930182T/A
5-1: 17959195A/C
5-1: 17983712T/C
5-1: 18124308A/G
5-1: 18152067A/G
5-1: 18267436T/C
5-1: 18280970G/A
5-1: 18329409C/G
5-1: 18379567A/C
5-1: 18379590T/C
5-1: 18433532G/A
5-1: 18475920T/C
5-1: 18509267A/G
5-1: 18554488C/A
5-1: 18573035C/T
5-1: 18646301G/T
5-1: 18758297G/A
5-1: 18768906A/T
5-1: 18795626G/A
5-1: 18807603A/G
5-1: 18873736G/T
5-1: 18924334C/T
5-1: 19066826T/C
5-1: 19144566G/A
5-1: 19161026A/C
5-1: 19192705T/G
5-1: 19241963G/T
5-1: 19244388A/G
5-1: 19276298A/T
5-1: 19319040A/G
5-1: 19322642C/T
5-1: 19391100A/G
5-1: 19435126A/G
5-1: 19453715C/T
5-1: 19488236C/T
5-1: 19694047T/A
5-1: 19700032T/A
5-1: 19726369G/A
5-1: 19757329C/T
5-1: 19781645C/A
5-1: 19801164C/T
5-1: 19804492G/A
5-1: 19813451C/T
5-1: 19840421C/T
5-1: 19866869T/C
5-1: 19884429T/C
5-1: 19890844T/G
5-1: 19906436G/A
5-1: 19932252A/G
5-1: 19969895A/G
5-1: 20080064T/C
5-1: 20091684G/A
5-1: 20104369T/C
5-1: 20162154A/G
5-1: 20188816A/G
5-1: 20320676C/T
5-1: 20342045A/G
5-1: 20378671C/T
5-1: 20402744T/C
5-1: 20404072G/T
5-1: 20445384A/G
5-1: 20476601C/T
5-1: 20488758T/A
5-1: 20506667C/A
5-1: 20613733G/A
5-1: 20687656G/A
5-1: 20721782G/T
5-1: 20776895C/G
5-1: 20797026T/C
5-1: 20806475C/G
5-1: 20808298T/C
5-1: 20826665T/C
5-1: 20833092T/A
5-1: 20833099A/T
5-1: 20837177A/G
5-1: 20848302T/C
5-1: 20865908C/T
5-1: 20959416T/C
5-1: 21006044T/A
5-1: 21033703T/A
5-1: 21137926G/A
5-1: 21195863T/C
5-1: 21197674C/T
5-1: 21198884A/G
5-1: 21232664G/A
5-1: 21244284T/G
5-1: 21287119T/A
5-1: 21318319C/G
5-1: 21450325G/T
5-1: 21495159C/T
5-1: 21617982A/T
5-1: 21651955T/C
5-1: 21677577C/T
5-1: 21696266G/A
5-1: 21725328G/T
5-1: 21736658G/A
5-1: 21753435C/A
5-1: 21754189G/A
5-1: 21755252G/T
5-1: 21891067C/T
5-1: 21904129G/T
5-1: 21915077T/C
5-1: 21915346T/C
5-1: 21932229C/G
5-1: 21957971A/C
5-1: 22013821T/A
5-1: 22072863G/T
5-1: 22086022C/T
5-1: 22097480C/A
5-1: 22176333A/G
5-1: 22187327C/T
5-1: 22224528T/C
5-1: 22236221A/G
5-1: 22249177A/G
5-1: 22275121T/C
5-1: 22301784T/G
5-1: 22312217G/A
5-1: 22344383C/T
5-1: 22357096C/T
5-1: 22406671A/T
5-1: 22448066A/G
5-1: 22471070G/A
5-1: 22491880A/T
5-1: 22504943C/T
5-1: 22515338G/T
5-1: 22529202G/C
5-1: 22547720C/T
5-1: 22567017G/A
5-1: 22573308T/C
5-1: 22584861A/G
5-1: 22596562G/A
5-1: 22673962C/A
5-1: 22674505C/T
5-1: 22674683A/G
5-1: 22675323T/C
5-1: 22675851C/T
5-1: 22759226A/T
5-1: 22817262A/G
5-1: 22825751G/C
5-1: 22825753T/A
5-1: 22828331C/T
5-1: 22842363T/C
5-1: 22861427G/A
5-1: 22912649T/A
5-1: 22924136C/A
5-1: 22955148C/T
5-1: 23009487T/C
5-1: 23055669G/A
5-1: 23066862A/G
5-1: 23122134A/G
5-1: 23174643C/T
5-1: 23184829T/G
5-1: 23197489T/C
5-1: 23224812G/A
5-1: 23235864C/T
5-1: 23236404A/T
5-1: 23246568C/G
5-1: 23248507G/A
5-1: 23256978G/C
5-1: 23279327A/G
5-1: 23299369C/T
5-1: 23310637A/G
5-1: 23464742T/A
5-1: 23473017T/C
5-1: 23492229T/A
5-1: 23499773T/C
5-1: 23577028A/C
5-1: 23620365A/G
5-1: 23684878T/C
5-1: 23697373C/A
5-1: 23722455C/T
5-1: 23734444C/T
5-1: 23746465C/G
5-1: 23760084T/C
5-1: 23778169G/A
5-1: 23788359C/T
5-1: 23793031G/A
5-1: 23806845G/A
5-1: 23807431A/G
5-1: 23818105T/A
5-1: 23837889C/A
5-1: 23850116T/C
5-1: 23856609C/T
5-1: 23860356T/C
5-1: 23860653T/C
5-1: 23902020C/A
5-1: 23957049G/T
5-1: 24040947C/T
5-1: 24053674T/A
5-1: 24077423T/G
5-1: 24111954C/T
5-1: 24147563A/G
5-1: 24182275T/C
5-1: 24199782C/T
5-1: 24211725G/A
5-1: 24238681C/T
5-1: 24282919T/C
5-1: 24320877C/T
5-1: 24330919T/A
5-1: 24372318C/G
5-1: 24417137T/C
5-1: 24430261A/G
5-1: 24442881G/C
5-1: 24556849G/A
5-1: 24655575T/C
5-1: 24666406T/G
5-1: 24678997A/G
5-1: 24689996A/G
5-1: 24705079C/T
5-1: 24724564T/A
5-1: 24773601T/C
5-1: 24789330C/T
5-1: 24813102T/A
5-1: 24836987G/A
5-1: 24841296T/A
5-1: 24841325T/A
5-1: 24841783A/C
5-1: 24853759T/A
5-1: 24882060C/A
5-1: 24915622T/C
5-1: 24920989G/T
5-1: 24921167A/T
5-1: 24925760G/C
5-1: 24941578T/C
5-1: 24944709C/T
5-1: 24944710T/C
5-1: 24960304G/T
5-1: 24974922G/C
5-1: 24985132G/A
5-1: 25015734T/A
5-1: 25066547A/G
5-1: 25082933T/A
5-1: 25103581T/C
5-1: 25126986G/T
5-1: 25131860A/G
5-1: 25132866G/A
5-1: 25133369G/A
5-1: 25139211G/T
5-1: 25166523G/T
5-1: 25167420T/G
5-1: 25196020T/A
5-1: 25207285A/C
5-1: 25207698G/A
5-1: 25207874C/G
5-1: 25221263T/C
5-1: 25221540C/T
5-1: 25225759C/T
5-1: 25225795G/A
5-1: 25232534G/A
5-1: 25242691G/A
5-1: 25243314A/G
5-1: 25258037C/T
5-1: 25290764G/T
5-1: 25305053T/C
5-1: 25307961T/C
5-1: 25308578G/A
5-1: 25336918C/G
5-1: 25370550A/T
5-1: 25370556C/A
5-1: 25370559G/A
5-1: 25370577A/G
5-1: 25370579T/C
5-1: 25371029C/T
5-1: 25371030G/A
5-1: 25371031G/A
5-1: 25371042G/A
5-1: 25371044G/T
5-1: 25371046A/C
5-1: 25371075C/T
5-1: 25371088T/C
5-1: 25371096T/.
5-1: 25371099A/C
5-1: 25371108C/G
5-1: 25371118C/A
5-1: 25371130A/G
5-1: 25371131A/G
5-1: 25371133C/.
5-1: 25371138T/C
5-1: 25371166A/C
5-1: 25371174C/T
5-1: 25371175G/A
5-1: 25371207G/A
5-1: 25371216C/A
5-1: 25371223A/G
5-1: 25371251A/T
5-1: 25371274T/C
5-1: 25371275T/C
5-1: 25415351A/G
5-1: 25415380A/G
5-1: 25415385C/T
5-1: 25415387A/C
5-1: 25415388C/T
5-1: 25415445A/C
5-1: 25415455G/A
5-1: 25415509C/T
5-1: 25415521A/T
5-1: 25415527C/T
5-1: 25415548A/T
5-1: 25415578C/T
5-1: 25415648A/C
5-1: 25415688G/A
5-1: 25415719A/G
5-1: 25415743A/G
5-1: 25415748G/A
5-1: 25415764A/G
5-1: 25415786G/A
5-1: 25415796G/A
5-1: 25415827A/G
5-1: 25415855G/A
5-1: 25415882G/C
5-1: 25415905T/G
5-1: 25415939A/G
5-1: 25416559C/T
5-1: 25416566C/G
5-1: 25416604G/A
5-1: 25423032T/C
5-1: 25423043C/T
5-1: 25423057A/G
5-1: 25423061A/T
5-1: 25423068T/A
5-1: 25423070T/A
5-1: 25423078G/T
5-1: 25423086A/T
5-1: 25423094C/T
5-1: 25423098T/C
5-1: 25423117A/G
5-1: 25423122G/A
5-1: 25423123A/C
5-1: 25423519G/C
5-1: 25423571T/C
5-1: 25423580T/C
5-1: 25423589C/T
5-1: 25423661G/C
5-1: 25433053C/G
5-1: 25444684T/A
5-1: 25459657A/G
5-1: 25498388G/A
5-1: 25529213T/A
5-1: 25542391A/T
5-1: 25562792A/T
5-1: 25568591C/T
5-1: 25608402G/A
5-1: 25675524C/T
5-1: 25719667T/G
5-1: 25757258G/A
5-1: 25798433G/A
5-1: 25810723T/G
5-1: 25821019T/C
5-1: 25822161G/A
5-1: 25921867G/A
5-1: 25927185T/G
5-1: 25998462A/G
5-1: 26000341T/C
5-1: 26000917C/T
5-1: 26017772C/G
5-1: 26031746C/T
5-1: 26069449T/G
5-1: 26097690G/A
5-1: 26114121G/T
5-1: 26141035A/G
5-1: 26202784C/A
5-1: 26214801A/T
5-1: 26243108T/C
5-1: 26260325C/T
5-1: 26265559C/A
5-1: 26271225C/T
5-1: 26329722T/G
5-1: 26356588T/C
5-1: 26401768C/T
5-1: 26408480A/C
5-1: 26408857T/C
5-1: 26480213G/T
5-1: 26501171G/A
5-1: 26538040A/G
5-1: 26550970T/C
5-1: 26561749C/A
5-1: 26654392C/G
5-1: 26655459T/G
5-1: 26663719G/A
5-1: 26666330G/A
5-1: 26679139G/C
5-1: 26679944A/C
5-1: 26684026C/T
5-1: 26706318G/A
5-1: 26734712G/A
5-1: 26801401A/G
5-1: 26812252T/G
5-1: 26826775G/A
5-1: 26853861A/G
5-1: 26866090A/G
5-1: 26866209G/A
5-1: 26866689A/G
5-1: 26866843G/A
5-1: 26909603A/C
5-1: 26952670C/T
5-1: 27042773G/A
5-1: 27065602G/C
5-1: 27069023T/C
5-1: 27069065C/T
5-1: 27069603C/T
5-1: 27070686C/A
5-1: 27071201T/C
5-1: 27082286G/A
5-1: 27085524T/C
5-1: 27119312C/T
5-1: 27201122G/A
5-1: 27212673T/C
5-1: 27240428C/T
5-1: 27348130C/T
5-1: 27384704T/G
5-1: 27391799A/G
5-1: 27391856A/G
5-1: 27397375T/G
5-1: 27397559G/T
5-1: 27409186C/T
5-1: 27416427G/T
5-1: 27416845C/T
5-1: 27434727C/A
5-1: 27459533C/T
5-1: 27485159A/G
5-1: 27528205T/C
5-1: 27528234T/A
5-1: 27528273A/G
5-1: 27528347C/G
5-1: 27528350A/G
5-1: 27528367A/G
5-1: 27528403T/A
5-1: 27530405C/T
5-1: 27543840G/A
5-1: 27564347C/T
5-1: 27589602C/T
5-1: 27590816G/T
5-1: 27591738T/C
5-1: 27609834A/T
5-1: 27620207T/C
5-1: 27654712T/G
5-1: 27684276T/A
5-1: 27721612G/A
5-1: 27735219A/G
5-1: 27753222C/A
5-1: 27817570A/C
5-1: 27828382G/A
5-1: 27844419T/G
5-1: 27858928C/T
5-1: 27883630T/A
5-1: 27899893A/G
5-1: 27913977G/A
5-1: 27918572C/T
5-1: 27924828G/T
5-1: 27926906G/A
5-1: 27929075T/G
5-1: 27944358G/A
5-1: 27974695G/A
5-1: 27987366G/A
5-1: 27997566C/T
5-1: 28008208C/T
5-1: 28025102G/A
5-1: 28066035G/A
5-1: 28151652A/G
5-1: 28186186A/C
5-1: 28200877G/A
5-1: 28211868G/T
5-1: 28243504T/C
5-1: 28276047A/G
5-1: 28286843G/A
5-1: 28297246A/T
5-1: 28302366T/A
5-1: 28304484G/C
5-1: 28310680A/T
5-1: 28405062A/G
5-1: 28508065G/A
5-1: 28511395A/T
5-1: 28563214G/A
5-1: 28584763G/A
5-1: 28599855A/G
5-1: 28628150G/T
5-1: 28639204C/T
5-1: 28641671C/T
5-1: 28653167A/G
5-1: 28669965G/A
5-1: 28714089G/A
5-1: 28731098T/C
5-1: 28780507G/A
5-1: 28873231T/G
5-1: 28887053G/A
5-1: 28898348A/G
5-1: 28910163G/A
5-1: 28910825A/T
5-1: 28935137C/G
5-1: 28970171T/C
5-1: 29054260A/G
5-1: 29067880A/G
5-1: 29073149C/A
5-1: 29095872C/A
5-1: 29108372C/A
5-1: 29113399C/T
5-1: 29119473A/T
5-1: 29165580G/A
5-1: 29180447T/A
5-1: 29202858G/T
5-1: 29259159A/.
5-1: 29259172G/A
5-1: 29259184G/C
5-1: 29259251G/C
5-1: 29259272C/T
5-1: 29259307G/C
5-1: 29259327C/T
5-1: 29259352G/.
5-1: 29259367C/A
5-1: 29259402T/C
5-1: 29259403A/G
5-1: 29262816T/A
5-1: 29274638C/T
5-1: 29338384G/T
5-1: 29354566A/G
5-1: 29355158T/A
5-1: 29355368T/A
5-1: 29355436C/T
5-1: 29355794C/T
5-1: 29355933A/T
5-1: 29356448C/G
5-1: 29356568T/C
5-1: 29356592T/C
5-1: 29356621A/T
5-1: 29356643A/C
5-1: 29356661G/A
5-1: 29356668A/T
5-1: 29356684T/G
5-1: 29356685T/C
5-1: 29356690C/A
5-1: 29359744C/T
5-1: 29366962G/A
5-1: 29381119G/T
5-1: 29401779T/C
5-1: 29419367G/C
5-1: 29471954T/C
5-1: 29482922G/A
5-1: 29511114G/A
5-1: 29535561T/C
5-1: 29560412T/C
5-1: 29561283C/T
5-1: 29587659A/G
5-1: 29615175T/C
5-1: 29623798G/T
5-1: 29626414T/C
5-1: 29638613T/C
5-1: 29651389G/A
5-1: 29668451A/G
5-1: 29715498G/A
5-1: 29722041T/A
5-1: 29730151G/A
5-1: 29742308T/A
5-1: 29868811G/A
5-1: 29878970G/A
5-1: 29895349A/G
5-1: 29901957G/T
5-1: 29904710A/G
5-1: 29905948G/T
5-1: 29908929C/T
5-1: 29929554G/A
5-1: 29944075C/T
5-1: 29964183G/A
5-1: 29964677G/A
5-1: 29965193G/A
5-1: 29965512C/T
5-1: 29991217G/A
5-1: 30006931C/G
5-1: 30017787A/G
5-1: 30019601G/A
5-1: 30089868A/G
5-1: 30100719T/A
5-1: 30115883C/T
5-1: 30142431A/T
5-1: 30177262A/G
5-1: 30199654A/G
5-1: 30211352G/A
5-1: 30266960C/G
5-1: 30294134T/G
5-1: 30424193G/A
5-1: 30482541G/A
5-1: 30498197G/A
5-1: 30518865G/A
5-1: 30535532A/C
5-1: 30604021G/A
5-1: 30620168C/T
5-1: 30638244G/A
5-1: 30750195A/G
5-1: 30754623T/A
5-1: 30786129C/T
5-1: 30826178T/C
5-1: 30880417C/T
5-1: 30881550C/T
5-1: 30881778G/T
5-1: 30884285G/T
5-1: 30885219T/C
5-1: 30885443G/A
5-1: 30897982A/G
5-1: 30907486G/T
5-1: 30913086G/C
5-1: 31049339G/A
5-1: 31095171G/A
5-1: 31114365G/A
5-1: 31134976G/A
5-1: 31179117T/G
5-1: 31226645T/C
5-1: 31274172G/T
5-1: 31306258C/T
5-1: 31377016G/C
5-1: 31389070G/A
5-1: 31458674C/T
5-1: 31503187T/C
5-1: 31534331T/C
5-1: 31547403T/C
5-1: 31557979T/G
5-1: 31613420T/C
5-1: 31625822C/T
5-1: 31671272T/C
5-1: 31699114C/T
5-1: 31711356A/G
5-1: 31734546A/T
5-1: 31751894C/T
5-1: 31759032T/G
5-1: 31763892A/T
5-1: 31763922G/A
5-1: 31763956T/C
5-1: 31764015T/C
5-1: 31764021C/T
5-1: 31764066C/.
5-1: 31764131A/C
5-1: 31764135T/C
5-1: 31764160G/A
5-1: 31764161G/A
5-1: 31764578A/.
5-1: 31764607C/T
5-1: 31764697G/A
5-1: 31764705G/T
5-1: 31777357T/C
5-1: 32191697C/T
5-1: 32211162T/C
5-1: 32277285A/T
5-1: 32294199A/G
5-1: 32304948C/T
5-1: 32351549C/A
5-1: 32397872C/T
5-1: 32419703G/T
5-1: 32630596G/A
5-1: 32719899G/A
5-1: 32736590T/C
5-1: 32750457A/G
5-1: 32761433A/C
5-1: 32807048G/C
5-1: 32855145C/A
5-1: 32862798C/T
5-1: 32996317G/C
5-1: 33154472A/G
5-1: 33224072T/C
5-1: 33290969G/A
5-1: 33327598G/A
5-1: 33519588T/A
5-1: 33524990T/A
5-1: 33656387A/T
5-1: 33675167A/G
5-1: 33699397C/T
5-1: 33725752C/G
5-1: 33745091C/T
5-1: 33764774C/T
5-1: 33800057C/T
5-1: 34771653A/T
5-1: 35015709C/A
5-1: 35137940C/T
5-1: 35141367A/G
5-1: 35229004A/G
5-1: 35247813A/G
5-1: 35265159C/T
5-1: 35267277G/T
5-1: 35313286T/A
5-1: 35338422A/G
5-1: 35343729C/T
5-1: 35364529A/C
5-1: 35401383A/T
5-1: 35473809C/T
5-1: 35517785G/C
5-1: 35534153C/T
5-1: 35582426T/C
5-1: 35582454T/A
5-1: 35625070C/G
5-1: 35703128C/T
5-1: 35781755G/T
5-1: 35783655C/T
5-1: 35787715G/A
5-1: 35787896T/C
5-1: 36014288C/T
5-1: 36167346C/A
5-1: 36187174C/T
5-1: 36306114G/A
5-1: 36359450T/C
5-1: 36387670C/T
5-1: 36398894G/A
5-1: 36417354T/A
5-1: 36430642C/T
5-1: 36471560T/C
5-1: 36530599C/T
5-1: 36543781T/A
5-1: 36639255C/A
5-1: 36731599A/T
5-1: 36830204G/C
5-1: 36841055A/G
5-1: 36898869A/T
5-1: 37007474T/C
5-1: 37025326C/T
5-1: 37045168C/T
5-1: 37088334G/T
5-1: 37088521A/G
5-1: 37136289C/T
5-1: 37191608C/T
5-1: 37208000G/A
5-1: 37232531C/A
5-1: 37246421T/C
5-1: 37308279G/A
5-1: 37356724T/C
5-1: 37411634A/G
5-1: 37506680C/T
5-1: 37557606C/A
5-1: 37617466A/G
5-1: 37653032T/G
5-1: 37686669T/G
5-1: 37706987C/T
5-1: 37765108T/C
5-1: 37791686G/T
5-1: 37804315A/G
5-1: 37804936A/G
5-1: 37815331A/G
5-1: 37849838T/A
5-1: 37849855A/G
5-1: 37852146A/G
5-1: 37854911T/A
5-1: 37860680C/T
5-1: 37896562C/T
5-1: 37933872C/T
5-1: 37979630G/A
5-1: 38030260T/A
5-1: 38041451G/C
5-1: 38073165T/G
5-1: 38120988C/T
5-1: 38242922G/A
5-1: 38331930G/T
5-1: 38403239G/A
5-1: 38450725C/G
5-1: 38531122C/G
5-1: 38612119T/A
5-1: 38633407A/C
5-1: 38648772T/A
5-1: 38709398G/A
5-1: 39089786A/G
5-1: 39092122C/T
5-1: 39102912G/A
5-1: 39127540C/T
5-1: 39183170T/A
5-1: 39206731A/T
5-1: 39208218C/T
5-1: 39256342G/A
5-1: 39283156G/A
5-1: 39343487A/G
5-1: 39363417T/C
5-1: 39392120A/G
5-1: 39402713G/C
5-1: 39415944A/G
5-1: 39427624T/C
5-1: 39542582C/T
5-1: 39549057A/G
5-1: 39552792C/T
5-1: 39572297C/A
5-1: 39626019T/C
5-1: 39650416C/T
5-1: 39774596G/A
5-1: 39854209T/G
5-1: 39938215C/A
5-1: 40007533C/T
5-1: 40042323A/G
5-1: 40153216T/C
5-1: 40191721C/T
5-1: 40275643T/C
5-1: 40306193G/A
5-1: 40350668A/G
5-1: 40395874A/G
5-1: 40467248G/C
5-1: 40484527C/G
5-1: 40512091A/G
5-1: 40561742T/A
5-1: 40659531G/T
5-1: 40725996G/A
5-1: 40737709A/G
5-1: 40765318T/C
5-1: 40805165A/G
5-1: 40842740T/C
5-1: 40853450G/A
5-1: 41027236T/C
5-1: 41038941T/G
5-1: 41100882T/A
5-1: 41122358C/T
5-1: 41143517A/C
5-1: 41143708A/G
5-1: 41199396C/T
5-1: 41199432T/C
5-1: 41199450C/A
5-1: 41251720A/T
5-1: 41265124C/A
5-1: 41312522A/G
5-1: 41432993T/C
5-1: 41448978T/C
5-1: 41490292G/A
5-1: 41550327C/T
5-1: 41563379C/T
5-1: 41696589A/T
5-1: 41825919A/G
5-1: 41827205G/T
5-1: 41840513T/C
5-1: 41921802G/T
5-1: 41957640T/C
5-1: 41971552A/G
5-1: 41974435T/C
5-1: 41984951A/T
5-1: 41997213T/C
5-1: 42038197A/T
5-1: 42050648T/C
5-1: 42052510A/G
5-1: 42071323T/C
5-1: 42081829C/A
5-1: 42102324G/A
5-1: 42106403T/G
5-1: 42114873G/C
5-1: 42136875G/A
5-1: 42153052G/C
5-1: 42292051C/A
5-1: 42297680C/T
5-1: 42312567C/T
5-1: 42324669A/G
5-1: 42335708T/A
5-1: 42464611C/T
5-1: 42512896G/A
5-1: 42543486G/A
5-1: 42566920C/T
5-1: 42640730C/G
5-1: 42711931T/C
5-1: 42772029G/T
5-1: 42828744T/A
5-1: 42869989A/C
5-1: 42881379G/A
5-1: 42911541C/A
5-1: 42981675C/A
5-1: 42992802C/T
5-1: 43091974T/C
5-1: 43104481G/C
5-1: 43148991C/A
5-1: 43231247T/A
5-1: 43259740G/A
5-1: 43286919T/A
5-1: 43529336C/T
5-1: 43655620G/A
5-1: 43700613A/C
5-1: 43817018T/A
5-1: 43879026G/T
5-1: 43879652T/A
5-1: 43906489A/T
5-1: 43922338T/G
5-1: 43964999T/C
5-1: 43990218G/A
5-1: 44012967T/C
5-1: 44056221C/T
5-1: 44153056C/T
5-1: 44191690T/C
5-1: 44253808T/C
5-1: 44360317T/A
5-1: 44411674C/T
5-1: 44447357A/T
5-1: 44471360A/T
5-1: 44525531A/G
5-1: 44561009A/T
5-1: 44579755G/A
5-1: 44663495T/C
5-1: 44690261A/G
5-1: 44752678A/T
5-1: 44770503T/G
5-1: 44829407G/A
5-1: 44829787C/T
5-1: 44865737A/G
5-1: 44876397T/C
5-1: 44880482T/C
5-1: 44943953C/T
5-1: 44943989C/G
5-1: 44944322C/T
5-1: 44944466A/G
5-1: 44944481G/A
5-1: 44944616G/A
5-1: 44944631G/A
5-1: 44967010T/C
5-1: 44982896T/C
5-1: 44992342T/C
5-1: 44992521C/T
5-1: 44993549A/G
5-1: 45004079A/C
5-1: 45019598T/C
5-1: 45056343C/T
5-1: 45151079A/C
5-1: 45161355T/C
5-1: 45267998G/T
5-1: 45341877G/A
5-1: 45362799A/G
5-1: 45495213A/G
5-1: 45524035C/T
5-1: 45562958G/T
5-1: 45609976C/T
5-1: 45658938T/C
5-1: 45697181C/A
5-1: 45708906G/A
5-1: 45759865T/C
5-1: 45787866A/G
5-1: 45810308T/C
5-1: 45812966T/A
5-1: 45842363A/G
5-1: 45922389T/C
5-1: 45989923C/T
5-1: 46059940T/A
5-1: 46095074T/A
5-1: 46115255G/A
5-1: 46127413G/A
5-1: 46138342G/A
5-1: 46157026A/G
5-1: 46168775C/A
5-1: 46181495G/A
5-1: 46193831T/A
5-1: 46207248A/T
5-1: 46251180C/G
5-1: 46315300C/T
5-1: 46385816T/C
5-1: 46412009G/A
5-1: 46424887G/C
5-1: 46439045G/A
5-1: 46453383A/G
5-1: 46481406T/C
5-1: 46494519G/A
5-1: 46560915T/A
5-1: 46600828C/T
5-1: 46605221G/A
5-1: 46605231C/G
5-1: 46609897A/T
5-1: 46617013C/T
5-1: 46710239G/A
5-1: 46730111G/A
5-1: 46740385G/A
5-1: 46751845G/A
5-1: 46796682A/T
5-1: 46824773C/T
5-1: 46877083T/C
5-1: 46909182C/T
5-1: 46974877G/A
5-1: 47004466C/T
5-1: 47051877T/C
5-1: 47085012T/A
5-1: 47110848T/G
5-1: 47138477A/T
5-1: 47165774A/G
5-1: 47197888T/A
5-1: 47233935C/T
5-1: 47331611T/C
5-1: 47409437C/T
5-1: 47413474T/G
5-1: 47452611C/T
5-1: 47532452T/C
5-1: 47616148C/T
5-1: 47689435T/C
5-1: 47759617T/A
5-1: 47761256C/T
5-1: 47781317C/T
5-1: 47856410A/G
5-1: 47868673G/C
5-1: 47904275T/C
5-1: 47924926C/A
5-1: 47975035T/G
5-1: 47988625G/A
5-1: 48011687C/T
5-1: 48018212A/C
5-1: 48035577C/T
5-1: 48059956C/T
5-1: 48061357T/A
5-1: 48061881C/T
5-1: 48063442C/T
5-1: 48082409C/T
5-1: 48104323G/T
5-1: 48144390A/T
5-1: 48243687T/G
5-1: 48278624C/T
5-1: 48339194T/C
5-1: 48417619G/A
5-1: 48460042A/G
5-1: 48466973C/T
5-1: 48516783C/A
5-1: 48543449C/G
5-1: 48608082T/G
5-1: 48642490G/T
5-1: 48686726G/A
5-1: 48775901C/T
5-1: 48828517A/G
5-1: 48955374G/C
5-1: 48958639C/T
5-1: 48963588A/G
5-1: 48965407A/C
5-1: 48966090C/T
5-1: 49013736A/T
5-1: 49033511T/C
5-1: 49061366T/C
5-1: 49120319G/T
5-1: 49135413C/T
5-1: 49300703C/G
5-1: 49321407A/G
5-1: 49333993C/T
5-1: 49398148C/T
5-1: 49403385C/T
5-1: 49410826T/C
5-1: 49426379G/T
5-1: 49461657G/A
5-1: 49479162C/A
5-1: 49528448C/A
5-1: 49563776C/T
5-1: 49623659A/C
5-1: 49634661A/T
5-1: 49658755A/T
5-1: 49698233G/C
5-1: 49724274G/A
5-1: 49813106A/C
5-1: 49839198T/C
5-1: 49871781G/A
5-1: 49895610A/G
5-1: 49985510A/T
5-1: 50004512A/G
5-1: 50026854C/T
5-1: 50037643T/C
5-1: 50038763A/G
5-1: 50038775G/A
5-1: 50071777G/A
5-1: 50097625A/C
5-1: 50165313C/T
5-1: 50232509T/C
5-1: 50236472C/T
5-1: 50352179T/C
5-1: 50393143G/A
5-1: 50411575C/T
5-1: 50422625A/G
5-1: 50437790A/G
5-1: 50464460G/A
5-1: 50495833G/A
5-1: 50548592T/C
5-1: 50616320A/T
5-1: 50648769A/G
5-1: 50669032G/A
5-1: 50701650G/T
5-1: 50762920T/A
5-1: 50783224C/G
5-1: 50796815T/G
5-1: 50811911T/C
5-1: 50868301G/A
5-1: 50956123A/T
5-1: 50997321A/T
5-1: 51008941G/A
5-1: 51102367A/G
5-1: 51143482A/G
5-1: 51178468A/C
5-1: 51223358G/C
5-1: 51253804C/G
5-1: 51336093T/G
5-1: 51366309A/C
5-1: 51417261A/T
5-1: 51457552A/C
5-1: 51500380G/A
5-1: 51530813G/A
5-1: 51535387C/T
5-1: 51536345A/G
5-1: 51537154T/C
5-1: 51596814G/A
5-1: 51641607C/G
5-1: 51762883T/C
5-1: 51814571A/G
5-1: 51847541A/G
5-1: 51857609C/T
5-1: 51896635C/T
5-1: 52084189G/A
5-1: 52098008G/A
5-1: 52289782G/A
5-1: 52443582A/G
5-1: 52454921T/C
5-1: 52498863C/T
5-1: 52576263C/T
5-1: 52577437G/A
5-1: 52578042C/T
5-1: 52646938G/T
5-1: 52676470G/A
5-1: 52881799A/C
5-1: 52887062C/T
5-1: 52908626C/A
5-1: 52918129C/A
5-1: 52931336C/T
5-1: 52955655A/C
5-1: 52980012A/G
5-1: 53015863T/A
5-1: 53027778G/A
5-1: 53068934C/A
5-1: 53099917G/A
5-1: 53130854T/C
5-1: 53184738G/A
5-1: 53259100T/C
5-1: 53294485A/G
5-1: 53320541C/T
5-1: 53323576T/C
5-1: 53357458C/T
5-1: 53387674G/A
5-1: 53415765G/A
5-1: 53512949A/G
5-1: 53599729C/T
5-1: 53668960C/T
5-1: 53679074G/T
5-1: 53764047C/T
5-1: 53775641T/G
5-1: 53793280C/G
5-1: 53861572A/G
5-1: 53915467G/T
5-1: 53995022C/T
5-1: 54053705C/T
5-1: 54167200T/G
5-1: 54182144A/G
5-1: 54216014A/T
5-1: 54306375T/C
5-1: 54336190A/G
5-1: 54414948G/A
5-1: 54484721T/G
5-1: 54508218G/A
5-1: 54509252T/C
5-1: 54527088G/A
5-1: 54599497A/G
5-1: 54610771A/G
5-1: 54621528T/C
5-1: 54647898G/C
5-1: 54690604T/G
5-1: 54723981T/G
5-1: 54754281G/A
5-1: 54779337G/A
5-1: 54783007G/T
5-1: 54804459T/C
5-1: 54838536A/C
5-1: 54855722A/G
5-1: 54866568C/T
5-1: 54885105A/T
5-1: 54899382C/G
5-1: 54913144T/G
5-1: 54944086C/A
5-1: 54971581G/A
5-1: 54989054A/G
5-1: 55001440A/T
5-1: 55013686C/A
5-1: 55039797A/T
5-1: 55171634G/A
5-1: 55188196G/A
5-1: 55200204G/T
5-1: 55322560T/C
5-1: 55417970G/A
5-1: 55440852A/C
5-1: 55622342T/A
5-1: 55690708G/A
5-1: 55708900C/T
5-1: 55728492C/T
5-1: 55777407C/T
5-1: 55778463C/A
5-1: 55787898G/A
5-1: 55800183T/A
5-1: 55856611T/A
5-1: 55883626T/C
5-1: 55972442T/C
5-1: 56001690T/C
5-1: 56053774A/G
5-1: 56066904A/T
5-1: 56139233T/G
5-1: 56155725G/C
5-1: 56170454G/A
5-1: 56200454G/A
5-1: 56246068G/A
5-1: 56274820T/C
5-1: 56301780T/C
5-1: 56303720C/T
5-1: 56336654C/T
5-1: 56404954A/T
5-1: 56440223A/C
5-1: 56466242C/T
5-1: 56487298A/G
5-1: 56531820T/C
5-1: 56551628T/G
5-1: 56581823T/C
5-1: 56640576G/T
5-1: 56706632C/A
5-1: 56708369G/A
5-1: 56733940G/T
5-1: 56735805T/C
5-1: 56771289A/T
5-1: 56783086A/C
5-1: 56784062G/A
5-1: 56858524A/G
5-1: 56974327A/G
5-1: 56975487T/A
5-1: 56994752T/C
5-1: 57007701T/A
5-1: 57062947T/G
5-1: 57094226T/A
5-1: 57122838A/G
5-1: 57174523A/G
5-1: 57261021T/C
5-1: 57278635T/C
5-1: 57338460C/T
5-1: 57375403A/G
5-1: 57406561G/C
5-1: 57420570A/G
5-1: 57464055T/C
5-1: 57484008A/C
5-1: 57514306G/A
5-1: 57534165A/G
5-1: 57535982C/T
5-1: 57551406A/G
5-1: 57571709T/C
5-1: 57573833T/A
5-1: 57617228C/T
5-1: 57630085A/G
5-1: 57647669A/G
5-1: 57693282T/G
5-1: 57705738G/A
5-1: 57720297A/G
5-1: 57732440T/C
5-1: 57737188T/C
5-1: 57737693T/G
5-1: 57742553A/T
5-1: 57742568T/C
5-1: 57742631C/T
5-1: 57753653C/A
5-1: 57776338A/T
5-1: 57793729T/C
5-1: 57809050T/G
5-1: 57810901T/C
5-1: 57846451G/A
5-1: 57865044T/C
5-1: 57892625C/G
5-1: 57898885G/A
5-1: 57899357C/T
5-1: 57900395A/C
5-1: 57902738T/C
5-1: 57913458A/G
5-1: 57939144T/C
5-1: 57986873G/T
5-1: 58015725T/G
5-1: 58058745C/A
5-1: 58088024G/A
5-1: 58121195C/T
5-1: 58129739A/G
5-1: 58174884T/G
5-1: 58186262C/T
5-1: 58202175C/G
5-1: 58245061A/G
5-1: 58245177G/C
5-1: 58313244C/T
5-1: 58314224T/G
5-1: 58353735C/T
5-1: 58434282A/G
5-1: 58454575C/T
5-1: 58474495A/G
5-1: 58528943T/C
5-1: 58570488T/G
5-1: 58609816A/G
5-1: 58709496T/C
5-1: 58759123A/G
5-1: 58785303A/G
5-1: 58849644A/C
5-1: 58885254C/A
5-1: 58908732A/T
5-1: 58942804C/T
5-1: 58976435T/C
5-1: 59035323C/T
5-1: 59092879G/A
5-1: 59109734G/T
5-1: 59144827T/C
5-1: 59191917A/T
5-1: 59214567A/G
5-1: 59229983G/A
5-1: 59230045C/T
5-1: 59364914T/C
5-1: 59396743C/T
5-1: 59540836G/A
5-1: 59545495G/A
5-1: 59581171C/T
5-1: 59621518G/A
5-1: 59631648A/T
5-1: 59647494G/A
5-1: 59697623T/A
5-1: 59772802G/A
5-1: 59823995G/T
5-1: 59872688A/T
5-1: 59887194A/C
5-1: 59939377A/G
5-1: 59939867C/T
5-1: 59955715G/A
5-1: 59998660G/A
5-1: 60097912A/T
5-1: 60130252G/T
5-1: 60233495G/A
5-1: 60268665A/T
5-1: 60345620G/A
5-1: 60444800C/G
5-1: 60541088C/T
5-1: 60575360T/C
5-1: 60660609T/C
5-1: 60702309G/T
5-1: 60713215A/G
5-1: 60815741C/A
5-1: 60826519G/A
5-1: 60863246G/A
5-1: 60891266A/G
5-1: 61035682A/C
5-1: 61064446C/T
5-1: 61068848T/C
5-1: 61084513C/T
5-1: 61116556G/A
5-1: 61117856C/A
5-1: 61117936G/T
5-1: 61136265G/A
5-1: 61208466A/G
5-1: 61221810C/A
5-1: 61253156C/T
5-1: 61263609A/C
5-1: 61274279G/A
5-1: 61386369A/G
5-1: 61396445G/A
5-1: 61408256T/C
5-1: 61434362A/G
5-1: 61487933T/C
5-1: 61530629C/T
5-1: 61652468A/T
5-1: 61658636T/G
5-1: 61672044A/T
5-1: 61701912A/G
5-1: 61730173C/T
5-1: 61790533T/A
5-1: 61891725T/C
5-1: 61947489T/C
5-1: 62000290A/C
5-1: 62031699C/G
5-1: 62103970G/A
5-1: 62131320A/G
5-1: 62163161T/C
5-1: 62211118G/T
5-1: 62285323G/A
5-1: 62325389T/C
5-1: 62388351G/A
5-1: 62420119T/C
5-1: 62502998G/A
5-1: 62597780A/C
5-1: 62660087A/T
5-1: 62711598G/A
5-1: 62739120T/C
5-1: 62759766A/G
5-1: 62791918G/T
5-1: 62809870A/C
5-1: 62857724A/T
5-1: 62923958T/C
5-1: 62984270G/T
5-1: 63001860A/G
5-1: 63050104G/T
5-1: 63062223T/C
5-1: 63106928A/G
5-1: 63137948T/C
5-1: 63158583C/T
5-1: 63185374T/C
5-1: 63269803G/T
5-1: 63360045G/A
5-1: 63425355A/G
5-1: 63461657A/G
5-1: 63495100A/T
5-1: 63620225T/G
5-1: 63643333C/T
5-1: 63683036T/A
5-1: 63699372A/T
5-1: 63723601G/A
5-1: 63769268G/T
5-1: 63782791T/C
5-1: 63858726A/G
5-1: 63901174G/A
5-1: 63975452C/T
5-1: 64020260A/G
5-1: 64050186C/T
5-1: 64110435T/A
5-1: 64160615G/T
5-1: 64239964A/T
5-1: 64250596T/C
5-1: 64331042G/A
5-1: 64439467G/T
5-1: 64453293C/G
5-1: 64466517G/T
5-1: 64483467C/T
5-1: 64577846A/G
5-1: 64751841A/T
5-1: 64824016C/T
5-1: 64870031G/A
5-1: 64922195G/A
5-1: 64927921A/T
5-1: 64971022A/C
5-1: 65070438T/C
5-1: 65126338T/A
5-1: 65178135C/T
5-1: 65179836A/T
5-1: 65208764G/A
5-1: 65260045T/C
5-1: 65261032T/C
5-1: 65311336T/C
5-1: 65554874C/T
5-1: 65596473C/A
5-1: 65646744C/A
5-1: 65722450T/C
5-1: 65800311A/G
5-1: 65814766A/C
5-1: 65817864A/T
5-1: 65831492G/A
5-1: 65987234T/A
5-1: 66020540A/G
5-1: 66034847T/C
5-1: 66070735G/A
5-1: 66129986C/T
5-1: 66140285G/C
5-1: 66336432A/G
5-1: 66380937T/G
5-1: 66431797C/T
5-1: 66434594G/A
5-1: 66481580G/A
5-1: 66645064C/T
5-1: 66694662G/A
5-1: 66713469G/A
5-1: 66755676T/G
5-1: 66841587G/A
5-1: 66855735C/T
5-1: 66907881A/T
5-1: 66923170G/A
5-1: 66947093C/T
5-1: 67177204A/C
5-1: 67403974C/T
5-1: 67667899G/C
5-1: 67763987A/T
5-1: 67799237G/A
5-1: 67838935C/T
5-1: 67875910G/T
5-1: 67918652T/C
5-1: 68044544C/T
5-1: 68051573G/A
5-1: 68099755G/A
5-1: 68113696G/A
5-1: 68261551G/A
5-1: 68264974T/A
5-1: 68265066C/A
5-1: 68265075C/T
5-1: 68265089A/G
5-1: 68475488C/G
5-1: 68511284C/T
5-1: 68606899C/G
5-1: 68991867G/A
5-1: 69155000C/T
5-1: 69167554G/A
5-1: 69172907A/T
5-1: 69177985G/A
5-1: 69296447C/G
5-1: 69300250A/G
5-1: 69603667A/T
5-1: 69671275G/A
5-1: 70374177A/G
5-1: 70510246T/C
5-1: 70717109T/G
5-1: 70719104A/G
5-1: 70727226A/G
5-1: 70727989T/G
5-1: 70732293C/T
5-1: 70739492T/C
5-1: 70740966G/C
5-1: 70754978C/T
5-1: 70756869G/A
5-1: 70792070A/G
5-1: 70802534C/T
5-1: 70838732A/G
5-1: 70918475A/C
5-1: 71121170C/T
5-1: 71287599T/C
5-1: 71381336T/C
5-1: 71541781A/G
5-1: 71846653G/C
5-1: 71860353T/C
5-1: 71861403A/C
5-2: 567142A/G
5-2: 641209A/T
5-2: 666459C/A
5-2: 804496T/A
5-2: 1004987A/C
5-2: 1005110T/C
5-2: 1005161C/A
5-2: 1227414T/C
5-2: 1228981T/A
5-2: 1231807T/A
5-2: 1231842A/T
5-2: 1231848T/C
5-2: 1231903C/T
5-2: 1231951A/T
5-2: 1276459C/T
5-2: 1391804A/G
5-2: 1415927C/G
5-2: 1432461T/G
5-2: 1651305A/T
5-2: 1821697G/C
5-2: 2019991C/T
5-2: 2064478A/T
5-2: 2104345G/A
5-2: 2270602C/G
5-2: 2544447A/C
5-2: 2754867G/A
5-2: 2869863G/A
5-2: 2889598C/T
5-2: 2939359C/A
5-2: 2999121G/A
5-2: 3182501G/C
5-2: 3435895C/T
5-2: 3443571G/A
5-2: 3538047G/A
5-2: 3568087G/T
5-2: 3606753A/T
5-2: 3754649G/C
5-2: 3828976A/T
5-2: 3899267G/A
5-2: 3910219T/C
5-2: 3916865T/A
5-2: 3956898G/C
5-2: 4005598T/C
5-2: 4032181G/T
5-2: 4059973G/A
5-2: 4071542G/A
5-2: 4090694C/T
5-2: 4168899A/G
5-2: 4234934A/T
5-2: 4245521T/C
5-2: 4268150A/G
5-2: 4307383A/G
5-2: 4353402A/T
5-2: 4385889T/G
5-2: 4449131T/A
5-2: 4481773T/A
5-2: 4546657G/T
5-2: 4551828A/G
5-2: 4585776G/C
5-2: 4656374A/G
5-2: 4775048G/A
5-2: 4875526G/A
5-2: 4914302A/C
5-2: 4959742C/A
5-2: 5031094C/T
5-2: 5100900C/T
5-2: 5197007A/T
5-2: 5203008A/G
5-2: 5348340A/G
5-2: 5401099G/A
5-2: 5425898C/T
5-2: 5523011A/T
5-2: 5602874G/A
5-2: 5631727A/G
5-2: 5713745G/A
5-2: 5787913G/T
5-2: 5842238A/T
5-2: 5854244T/C
5-2: 5865987C/T
5-2: 5939328A/G
5-2: 6013553T/C
5-2: 6126072C/T
5-2: 6184481C/T
5-2: 6254450G/T
5-2: 6343945T/A
5-2: 6398411G/A
5-2: 6418114C/G
5-2: 6493029A/G
5-2: 6545225A/T
5-2: 6609483C/T
5-2: 6636100C/T
5-2: 6796216A/G
5-2: 6834619C/T
5-2: 6950755C/T
5-2: 6984411G/A
5-2: 7039815A/C
5-2: 7064363C/T
5-2: 7176166A/G
5-2: 7218122T/C
5-2: 7299819G/A
5-2: 7299966C/T
5-2: 7300176G/C
5-2: 7328238T/C
5-2: 7340018G/A
5-2: 7343395T/A
5-2: 7345719G/A
5-2: 7364073T/C
5-2: 7382446G/A
5-2: 7388150T/C
5-2: 7389932G/T
5-2: 7392105C/T
5-2: 7392766T/C
5-2: 7401463A/C
5-2: 7404656A/G
5-2: 7423185C/G
5-2: 7445981A/T
5-2: 7452519T/C
5-2: 7490567C/T
5-2: 7506921G/T
5-2: 7540146C/T
5-2: 7572521C/A
5-2: 7615556C/T
5-2: 7638519C/A
5-2: 7775359T/C
5-2: 7872393C/T
5-2: 8022266A/C
5-2: 8148244C/T
5-2: 8187662T/A
5-2: 8414307C/T
5-2: 8454717T/A
5-2: 8506634G/A
5-2: 8541797A/T
5-2: 8592900G/T
5-2: 8640876A/G
5-2: 8683083A/C
5-2: 8729820A/G
5-2: 8750436A/G
5-2: 8799560T/C
5-2: 8901200T/C
5-2: 8958226A/G
5-2: 9038157C/G
5-2: 9099597A/G
5-2: 9108506A/G
5-2: 9108672C/T
5-2: 9108719G/A
5-2: 9108722A/T
5-2: 9108791T/C
5-2: 9174558T/C
5-2: 9191872A/T
5-2: 9197255T/G
5-2: 9217398T/A
5-2: 9252333T/C
5-2: 9289825C/T
5-2: 9483751A/G
5-2: 9669183T/A
5-2: 9705659A/C
5-2: 9717304C/A
5-2: 9729864C/T
5-2: 9744215A/T
5-2: 9772702A/C
5-2: 9873697G/C
5-2: 9909670T/C
5-2: 9990282T/A
5-2: 10098544C/T
5-2: 10099594C/A
5-2: 10139361A/T
5-2: 10224114A/G
5-2: 10256235C/A
5-2: 10320754A/C
5-2: 10432319A/G
5-2: 10491126G/C
5-2: 10514612A/G
5-2: 10526931A/C
5-2: 10588720G/A
5-2: 10658176A/C
5-2: 10689678T/G
5-2: 10734871G/A
5-2: 10782990C/T
5-2: 10860137C/T
5-2: 10965723A/G
5-2: 11016319G/A
5-2: 11085844C/T
5-2: 11161124C/T
5-2: 11283555A/G
5-2: 11337853T/C
5-2: 11419248T/A
5-2: 11434291A/G
5-2: 11464383T/C
5-2: 11574798T/C
5-2: 11578269G/A
5-2: 11618298C/T
5-2: 11680249C/T
5-2: 11767840C/T
5-2: 11841165G/A
5-2: 11858201T/A
5-2: 11862545C/T
5-2: 12005568G/A
5-2: 12050499C/T
5-2: 12054194A/T
5-2: 12117040C/A
5-2: 12385707A/G
5-2: 12523559C/T
5-2: 12584009C/T
5-2: 12667373T/C
5-2: 12716601C/T
5-2: 12743637C/T
5-2: 12905060T/C
5-2: 13058168T/C
5-2: 13061656G/A
5-2: 13098313G/T
5-2: 13339956G/A
5-2: 13438156G/A
5-2: 13538887C/T
5-2: 13582496A/T
5-2: 13620991A/T
5-2: 13655679A/G
5-2: 13702281C/A
5-2: 13713337A/G
5-2: 13778761T/A
5-2: 13844111T/C
5-2: 13940751C/T
5-2: 13978321T/C
5-2: 14017863T/G
5-2: 14032336C/T
5-2: 14060375A/C
5-2: 14120664G/C
5-2: 14297687C/T
5-2: 14347878A/G
5-2: 14394830A/G
5-2: 14442104C/T
5-2: 14470159T/G
5-2: 14499952T/C
5-2: 14511827G/A
5-2: 14560990T/C
5-2: 14599796G/T
5-2: 14669947C/A
5-2: 14722011G/A
5-2: 14798436C/T
5-2: 14897783A/G
5-2: 14908267A/G
5-2: 14941722A/T
5-2: 14971111C/T
5-2: 15011423C/T
5-2: 15101465C/T
5-2: 15141604C/T
5-2: 15184904T/A
5-2: 15251244C/T
5-2: 15284696C/T
5-2: 15363200G/A
5-2: 15391318T/A
5-2: 15449424G/C
5-2: 15522653C/T
5-2: 15564622T/C
5-2: 15580255T/C
5-2: 15625489A/G
5-2: 15801253C/T
5-2: 15872334A/T
5-2: 15919428G/A
5-2: 16023990T/C
5-2: 16081507G/A
5-2: 16276455A/C
5-2: 16339530C/T
5-2: 16386352T/C
5-2: 16463402G/A
5-2: 16576098C/T
5-2: 16646156T/C
5-2: 16647186C/T
5-2: 16746705C/T
5-2: 16761141G/A
5-2: 16775120A/G
5-2: 16808958A/G
5-2: 16821186C/T
5-2: 16879770A/T
5-2: 16889848C/T
5-2: 16905497A/C
5-2: 16926599A/T
5-2: 16942897C/A
5-2: 17160772G/T
5-2: 17183363G/A
5-2: 17224624G/A
5-2: 17254752G/T
5-2: 17291255T/C
5-2: 17397260T/C
5-2: 17494569C/T
5-2: 17546120C/T
5-2: 17674671G/A
5-2: 17705522A/G
5-2: 17715955A/G
5-2: 17766162A/G
5-2: 17811833T/G
5-2: 17860781G/T
5-2: 17887513A/T
5-2: 18005055T/G
5-2: 18015586G/T
5-2: 18040588G/T
5-2: 18159334T/A
5-2: 18187481A/G
5-2: 18188806G/A
5-2: 18189337G/A
5-2: 18225007G/A
5-2: 18313346T/G
5-2: 18330717A/G
5-2: 18335937T/C
5-2: 18338786T/C
5-2: 18362881G/A
5-2: 18439654A/G
5-2: 18471360A/G
5-2: 18488024A/C
5-2: 18526659G/A
5-2: 18561647C/T
5-2: 18573129C/A
5-2: 18608177A/G
5-2: 18619879A/G
5-2: 18641216A/G
5-2: 18700311A/G
5-2: 18741383G/A
5-2: 18804339C/A
5-2: 18871318T/A
5-2: 18973471G/T
5-2: 18983476G/A
5-2: 18999937A/G
5-2: 19035912G/C
5-2: 19055107A/G
5-2: 19085298C/T
5-2: 19151801C/T
5-2: 19189823G/A
5-2: 19244135C/T
5-2: 19258275A/T
5-2: 19308706A/G
5-2: 19334066C/T
5-2: 19380469T/A
5-2: 19394584A/C
5-2: 19412343G/A
5-2: 19476874G/T
5-2: 19529968G/C
5-2: 19597237G/A
5-2: 19680142G/A
5-2: 19683697C/T
5-2: 19684491C/G
5-2: 19747978T/C
5-2: 19762288A/G
5-2: 20026872T/C
5-2: 20164080A/T
5-2: 20177925C/A
5-2: 20190550G/A
5-2: 20226214G/A
5-2: 20260510G/T
5-2: 20293613T/A
5-2: 20386865G/A
5-2: 20387745C/T
5-2: 20387874C/T
5-2: 20388793C/A
5-2: 20413096A/G
5-2: 20432332C/T
5-2: 20485161C/T
5-2: 20514515A/G
5-2: 20563081C/T
5-2: 20620242A/T
5-2: 20622125C/G
5-2: 20671160G/T
5-2: 20835336A/G
5-2: 20863193A/G
5-2: 20913032T/C
5-2: 20952415C/A
5-2: 21149123A/G
5-2: 21214956T/C
5-2: 21256373A/C
5-2: 21358216A/G
5-2: 21393195A/T
5-2: 21404876C/A
5-2: 21420863A/T
5-2: 21479944C/T
5-2: 21501825T/A
5-2: 21620604C/A
5-2: 21754813C/T
5-2: 21758433C/T
5-2: 21771309G/T
5-2: 21790934C/A
5-2: 21903846C/T
5-2: 21947590G/T
5-2: 21999469A/G
5-2: 22060296C/A
5-2: 22081266T/C
5-2: 22118772T/C
5-2: 22145328A/C
5-2: 22206312A/C
5-2: 22226675T/A
5-2: 22244654T/C
5-2: 22275594G/A
5-2: 22298965A/G
5-2: 22317119T/C
5-2: 22318839C/T
5-2: 22327567T/C
5-2: 22347883T/C
5-2: 22368963A/C
5-2: 22409164G/A
5-2: 22497362A/G
5-2: 22511228A/T
5-2: 22523990A/G
5-2: 22526575A/G
5-2: 22583361T/C
5-2: 22584891A/C
5-2: 22631130C/G
5-2: 22651612C/G
5-2: 22702743C/T
5-2: 22777759A/C
5-2: 22817460T/C
5-2: 22844635A/G
5-2: 22871606A/T
5-2: 22902751T/A
5-2: 22932415A/G
5-2: 22953029C/T
5-2: 22976016A/G
5-2: 22987128T/C
5-2: 23070164T/C
5-2: 23122467T/C
5-2: 23231153G/A
5-2: 23266176T/G
5-2: 23278408C/T
5-2: 23415916C/T
5-2: 23425981C/T
5-2: 23446677C/T
5-2: 23508239C/T
5-2: 23579253T/C
5-2: 23606315T/C
5-2: 23666215C/A
5-2: 23709132G/A
5-2: 23741107C/G
5-2: 23775042G/A
5-2: 23811158G/A
5-2: 23839063A/G
5-2: 23911020T/A
5-2: 23929605G/A
5-2: 23999344G/A
5-2: 24070145C/T
5-2: 24097775T/C
5-2: 24115108G/A
5-2: 24130591T/C
5-2: 24147218A/T
5-2: 24170200T/C
5-2: 24190650T/A
5-2: 24251086C/G
5-2: 24292396G/A
5-2: 24376579C/.
5-2: 24381223A/T
5-2: 24440984T/A
5-2: 24553678C/T
5-2: 24589718T/A
5-2: 24590776G/A
5-2: 24596317C/T
5-2: 24596829A/G
5-2: 24597343T/C
5-2: 24597608A/T
5-2: 24598267G/A
5-2: 24598912A/C
5-2: 24599076C/T
5-2: 24599182A/G
5-2: 24602985T/A
5-2: 24673283A/C
5-2: 24715146A/G
5-2: 24727835C/A
5-2: 24755571G/A
5-2: 24766008A/G
5-2: 24795308A/T
5-2: 24827216G/A
5-2: 24838309C/A
5-2: 24919450C/T
5-2: 24946709A/G
5-2: 24979879G/A
5-2: 25002713G/A
5-2: 25057942C/T
5-2: 25086944T/C
5-2: 25097282C/G
5-2: 25110765A/G
5-2: 25140456C/A
5-2: 25164323C/G
5-2: 25176303G/A
5-2: 25196014T/A
5-2: 25208556T/A
5-2: 25221453G/A
5-2: 25237049A/G
5-2: 25267528C/T
5-2: 25277731A/T
5-2: 25277733G/A
5-2: 25277786G/A
5-2: 25277798C/T
5-2: 25277845T/.
5-2: 25277867A/G
5-2: 25278079C/A
5-2: 25278405A/G
5-2: 25282680C/T
5-2: 25289312T/C
5-2: 25299779A/G
5-2: 25302073A/G
5-2: 25302119C/T
5-2: 25302639T/C
5-2: 25302730C/T
5-2: 25303235T/C
5-2: 25311306T/A
5-2: 25312250C/T
5-2: 25318486C/T
5-2: 25318858T/A
5-2: 25319014A/G
5-2: 25319371C/T
5-2: 25319553G/A
5-2: 25322945G/C
5-2: 25323012C/T
5-2: 25331907C/T
5-2: 25333517G/C
5-2: 25346001A/C
5-2: 25356649C/T
5-2: 25377599C/T
5-2: 25391700C/T
5-2: 25396267G/A
5-2: 25415806C/T
5-2: 25479179T/C
5-2: 25493489T/C
5-2: 25504451A/T
5-2: 25528297G/A
5-2: 25541814T/A
5-2: 25548853G/C
5-2: 25585551A/T
5-2: 25640752A/G
5-2: 25661638T/C
5-2: 25720481G/C
5-2: 25748069C/A
5-2: 25760105T/G
5-2: 25770365T/C
5-2: 25770534C/T
5-2: 25770884A/G
5-2: 25779220A/G
5-2: 25786418C/T
5-2: 25813848T/A
5-2: 25841973T/A
5-2: 25881261T/C
5-2: 25892339T/A
5-2: 25909747A/G
5-2: 25953087C/T
5-2: 25986172G/A
5-2: 26009767T/A
5-2: 26029859G/A
5-2: 26081536G/A
5-2: 26130207T/G
5-2: 26159522A/C
5-2: 26198655A/G
5-2: 26227626C/T
5-2: 26309025C/A
5-2: 26309834G/A
5-2: 26320509A/G
5-2: 26325422C/T
5-2: 26326531A/G
5-2: 26418796A/G
5-2: 26457684A/T
5-2: 26469468A/G
5-2: 26519910T/C
5-2: 26550763A/C
5-2: 26552680T/C
5-2: 26566282G/A
5-2: 26608702A/T
5-2: 26620600T/C
5-2: 26623430C/T
5-2: 26631978G/T
5-2: 26644190T/C
5-2: 26743612T/C
5-2: 26768249T/G
5-2: 26782697G/T
5-2: 26793527C/A
5-2: 26794260A/T
5-2: 26825995A/T
5-2: 26875777C/T
5-2: 26884415A/G
5-2: 26914177C/A
5-2: 26916767C/T
5-2: 26918281A/G
5-2: 26932445A/G
5-2: 26966383A/C
5-2: 26981296T/A
5-2: 27012814T/G
5-2: 27025591T/C
5-2: 27043325G/A
5-2: 27068981C/T
5-2: 27113458T/G
5-2: 27143389C/T
5-2: 27188544T/A
5-2: 27228751A/T
5-2: 27259795A/C
5-2: 27262996C/T
5-2: 27339756G/T
5-2: 27359497C/G
5-2: 27406444T/C
5-2: 27423264T/G
5-2: 27449525T/A
5-2: 27464808A/G
5-2: 27466006T/C
5-2: 27475468T/C
5-2: 27482675T/G
5-2: 27495497A/G
5-2: 27499611A/T
5-2: 27499909A/G
5-2: 27507427C/T
5-2: 27526701T/C
5-2: 27545743G/A
5-2: 27555789T/C
5-2: 27570896G/T
5-2: 27582734T/A
5-2: 27604281T/C
5-2: 27635158A/G
5-2: 27666558T/C
5-2: 27684469A/C
5-2: 27701647C/A
5-2: 27708429G/A
5-2: 27713156G/A
5-2: 27764634C/T
5-2: 27819333C/G
5-2: 27821584C/.
5-2: 27838438A/C
5-2: 27840180G/A
5-2: 27852219A/C
5-2: 27855049A/G
5-2: 27855060T/C
5-2: 27855146A/C
5-2: 27855222T/A
5-2: 27855267G/A
5-2: 27855356G/A
5-2: 27855444G/A
5-2: 27855449A/G
5-2: 27855471A/G
5-2: 27855497G/A
5-2: 27855525T/G
5-2: 27855897T/C
5-2: 27862196A/G
5-2: 27876934A/T
5-2: 27880315C/T
5-2: 27887628A/T
5-2: 27896881A/T
5-2: 27897812A/G
5-2: 27901597G/A
5-2: 27911477T/C
5-2: 27946195T/C
5-2: 27948936G/A
5-2: 27958821C/T
5-2: 28035240T/G
5-2: 28096548C/T
5-2: 28160901A/G
5-2: 28188778C/T
5-2: 28241160C/T
5-2: 28251673A/G
5-2: 28252764T/C
5-2: 28253456G/C
5-2: 28257420C/A
5-2: 28308390C/T
5-2: 28329369C/T
5-2: 28355803A/G
5-2: 28393170T/C
5-2: 28402908A/G
5-2: 28403991G/A
5-2: 28466663T/C
5-2: 28495975C/G
5-2: 28568844T/C
5-2: 28606380T/C
5-2: 28639746A/G
5-2: 28650324T/C
5-2: 28652122T/C
5-2: 28664816G/C
5-2: 28765731G/A
5-2: 28766773G/C
5-2: 28781828A/G
5-2: 28801722A/G
5-2: 28821370A/C
5-2: 28863895A/C
5-2: 28870231G/A
5-2: 28903301C/T
5-2: 28942019G/A
5-2: 28964244C/A
5-2: 28989842A/G
5-2: 29068443A/G
5-2: 29080564T/C
5-2: 29092078A/T
5-2: 29107973G/A
5-2: 29108035T/C
5-2: 29119564T/G
5-2: 29183716G/A
5-2: 29186009C/T
5-2: 29254045C/T
5-2: 29265496T/C
5-2: 29267238A/G
5-2: 29267275C/T
5-2: 29267293C/T
5-2: 29267346A/C
5-2: 29267354T/C
5-2: 29267374T/G
5-2: 29267441T/.
5-2: 29267518G/A
5-2: 29267540C/T
5-2: 29267628T/C
5-2: 29267648A/G
5-2: 29267696C/T
5-2: 29267698A/T
5-2: 29267727T/G
5-2: 29267811C/G
5-2: 29267831A/G
5-2: 29268236T/C
5-2: 29268238T/C
5-2: 29268245G/A
5-2: 29268257G/A
5-2: 29268300T/A
5-2: 29268365A/T
5-2: 29276318G/A
5-2: 29292448C/T
5-2: 29320742A/G
5-2: 29405247C/A
5-2: 29421541G/A
5-2: 29464374T/G
5-2: 29489694A/G
5-2: 29507197G/A
5-2: 29530219G/T
5-2: 29544044G/T
5-2: 29623147C/T
5-2: 29654651G/T
5-2: 29659060A/T
5-2: 29661826A/G
5-2: 29662177T/G
5-2: 29681319T/C
5-2: 29750563C/G
5-2: 29787387T/A
5-2: 29799158A/T
5-2: 29843805G/A
5-2: 29866787G/C
5-2: 29895432C/T
5-2: 29967720G/A
5-2: 29995859C/T
5-2: 30030773T/G
5-2: 30073294C/T
5-2: 30083698T/C
5-2: 30113848C/G
5-2: 30132074T/A
5-2: 30135309T/C
5-2: 30171695T/C
5-2: 30210782T/C
5-2: 30269012G/A
5-2: 30281158T/C
5-2: 30300877A/C
5-2: 30312915A/G
5-2: 30313074T/A
5-2: 30334200C/G
5-2: 30380856T/C
5-2: 30392140C/T
5-2: 30427382G/C
5-2: 30453144T/C
5-2: 30467801C/T
5-2: 30480856G/A
5-2: 30491131A/G
5-2: 30511910A/G
5-2: 30547678A/G
5-2: 30599163C/T
5-2: 30776156C/T
5-2: 30911661A/T
5-2: 30940012A/G
5-2: 31049070C/T
5-2: 31309277T/C
5-2: 31436961T/C
5-2: 31529938C/T
5-2: 31693152T/C
5-2: 31954860G/C
5-2: 32115172A/G
5-2: 32474306T/C
5-2: 32510712G/A
5-2: 32608835G/A
5-2: 32690256G/A
5-2: 32837070G/T
5-2: 33096505A/T
5-2: 33160384G/T
5-2: 33331143G/A
5-2: 33376200G/A
5-2: 33883073C/T
5-2: 33933429G/A
5-2: 34229556T/C
5-2: 34241495C/T
5-2: 34527937C/T
5-2: 34538041T/C
5-2: 34560619C/T
5-2: 34589310T/G
5-2: 34590437G/C
5-2: 34606113G/A
5-2: 34633825C/T
5-2: 34653618G/A
5-2: 34676950T/C
5-2: 34694503C/A
5-2: 34710777T/C
5-2: 34727438G/A
5-2: 34764540C/T
5-2: 34786598G/T
5-2: 34799745A/T
5-2: 34868067T/C
5-2: 34893253G/A
5-2: 34923983T/C
5-2: 34927202T/G
5-2: 34929074C/T
5-2: 34934541T/G
5-2: 34945073C/T
5-2: 34963295C/T
5-2: 35089265T/C
5-2: 35113659G/C
5-2: 35241577A/C
5-2: 35261094A/G
5-2: 35284202A/T
5-2: 35381245C/G
5-2: 35402064C/T
5-2: 35412733A/G
5-2: 35432389A/T
5-2: 35567655T/C
5-2: 35615169C/G
5-2: 35825203T/C
5-2: 35888126C/T
5-2: 35955570G/A
5-2: 36047451C/T
5-2: 36117981C/T
5-2: 36122711A/T
5-2: 36123107T/C
5-2: 36133849C/A
5-2: 36175297G/A
5-2: 36200774T/A
5-2: 36240481A/G
5-2: 36252554C/T
5-2: 36278279T/A
5-2: 36352788G/A
5-2: 36411230C/T
5-2: 36441693G/A
5-2: 36527629A/G
5-2: 36587732G/C
5-2: 36593518G/A
5-2: 36625940G/A
5-2: 36651056T/C
5-2: 36688244G/A
5-2: 36729762A/T
5-2: 36755565C/T
5-2: 36790821A/T
5-2: 36801180C/T
5-2: 36830650T/A
5-2: 36856886C/T
5-2: 36881943T/C
5-2: 36917021C/T
5-2: 36955891G/A
5-2: 36990485C/G
5-2: 37089495C/T
5-2: 37101219T/A
5-2: 37105326T/C
5-2: 37130706C/T
5-2: 37181187A/G
5-2: 37240652G/T
5-2: 37367076C/T
5-2: 37408578A/G
5-2: 37422569G/A
5-2: 37524394T/A
5-2: 37536633C/T
5-2: 37538787G/A
5-2: 37793681T/A
5-2: 37857724A/C
5-2: 37876924C/T
5-2: 38152675T/C
5-2: 38247619C/T
5-2: 38330523C/T
5-2: 38543819T/C
5-2: 38672788G/A
5-2: 38693048G/A
5-2: 38753451C/T
5-2: 38784283C/T
5-2: 38820212G/A
5-2: 39044014A/T
5-2: 39054896T/C
5-2: 39068249G/A
5-2: 39085764A/T
5-2: 39100026G/A
5-2: 39116712C/G
5-2: 39175213T/G
5-2: 39415711G/A
5-2: 39419187T/A
5-2: 39427527A/C
5-2: 39535176T/C
5-2: 39554088A/G
5-2: 39700015G/A
5-2: 39821874G/A
5-2: 39834072T/A
5-2: 39845433C/T
5-2: 39846823T/C
5-2: 39857295G/T
5-2: 39879973A/G
5-2: 39884431C/T
5-2: 39900541C/T
5-2: 39926507C/A
5-2: 39984888C/T
5-2: 40001254C/T
5-2: 40016938T/A
5-2: 40042778C/T
5-2: 40088835G/A
5-2: 40169045C/T
5-2: 40217170C/A
5-2: 40233650T/C
5-2: 40304766C/T
5-2: 40376570C/A
5-2: 40401994T/C
5-2: 40419539A/G
5-2: 40475088T/C
5-2: 40502913T/C
5-2: 40665496A/T
5-2: 40874667G/T
5-2: 40875742A/G
5-2: 40893838G/A
5-2: 40915925G/C
5-2: 40946494A/G
5-2: 40964424T/A
5-2: 40964725A/G
5-2: 40964770C/T
5-2: 40964781T/C
5-2: 40964786C/T
5-2: 41142640T/G
5-2: 41144549G/A
5-2: 41188448C/T
5-2: 41229636C/T
5-2: 41359015C/T
5-2: 41408904T/A
5-2: 41424686G/C
5-2: 41455558G/A
5-2: 41493779A/T
5-2: 41512476A/G
5-2: 41516829A/C
5-2: 41571971G/T
5-2: 41592692A/G
5-2: 41621114G/T
5-2: 41798705A/T
5-2: 41831626A/C
5-2: 41832750A/G
5-2: 41867626T/A
5-2: 41869582G/A
5-2: 41890039T/C
5-2: 41949554C/A
5-2: 41964160G/A
5-2: 41995018C/T
5-2: 42056874C/T
5-2: 42091844T/G
5-2: 42138492G/T
5-2: 42158481G/A
5-2: 42176937C/T
5-2: 42240725A/G
5-2: 42315158G/A
5-2: 42426390A/G
5-2: 42521244C/G
5-2: 42567838A/G
5-2: 42649494G/A
5-2: 42676285G/A
5-2: 42677802G/A
5-2: 42745020T/C
5-2: 42774077C/A
5-2: 42817547T/A
5-2: 42832995C/T
5-2: 42879791C/T
5-2: 42886157T/C
5-2: 42947771G/A
5-2: 42986938T/A
5-2: 43003736T/C
5-2: 43050850G/A
5-2: 43120070C/A
5-2: 43185436T/C
5-2: 43281924A/T
5-2: 43301432A/G
5-2: 43325857C/A
5-2: 43393625T/C
5-2: 43408231A/C
5-2: 43430789G/A
5-2: 43464932G/A
5-2: 43522559T/G
5-2: 43621610C/G
5-2: 43701065C/A
5-2: 43728014A/G
5-2: 43787801A/G
5-2: 43817199G/T
5-2: 43872011G/A
5-2: 43902371T/A
5-2: 43956638T/C
5-2: 44037278A/C
5-2: 44115493C/T
5-2: 44144825C/T
5-2: 44182175T/C
5-2: 44193562C/T
5-2: 44210709G/A
5-2: 44221778C/T
5-2: 44280056T/G
5-2: 44311767A/C
5-2: 44398739C/T
5-2: 44422240A/G
5-2: 44429277C/G
5-2: 44439401A/G
5-2: 44514726A/G
5-2: 44573355G/A
5-2: 44646159C/A
5-2: 44656703C/T
5-2: 44674091C/T
5-2: 44699510G/A
5-2: 44753219G/A
5-2: 44754736C/T
5-2: 44786587C/G
5-2: 44832539C/T
5-2: 44853951A/G
5-2: 44898967C/T
5-2: 44923379T/G
5-2: 44950522A/T
5-2: 45028252A/G
5-2: 45029982A/T
5-2: 45060437A/C
5-2: 45089435G/A
5-2: 45134528A/C
5-2: 45174047G/A
5-2: 45176664G/A
5-2: 45251147G/C
5-2: 45307676A/T
5-2: 45375906C/T
5-2: 45457053A/G
5-2: 45482547A/G
5-2: 45531180G/A
5-2: 45562397A/G
5-2: 45611231T/C
5-2: 45651318A/T
5-2: 45681927C/T
5-2: 45699155G/T
5-2: 45713445C/A
5-2: 45713949T/C
5-2: 45713962A/T
5-2: 45737215G/A
5-2: 45792001A/C
5-2: 45856583T/G
5-2: 45940220T/C
5-2: 45950224C/T
5-2: 45951549T/A
5-2: 45963728C/T
5-2: 46114571T/C
5-2: 46406316A/T
5-2: 46480095A/C
5-2: 46522952A/G
5-2: 46575015G/A
5-2: 46636160A/G
5-2: 46651193G/A
5-2: 46719130A/G
5-2: 46745325C/T
5-2: 46842920C/T
5-2: 46889290T/C
5-2: 46947405T/A
5-2: 46976701C/T
5-2: 47023116G/A
5-2: 47106959T/C
5-2: 47228328G/A
5-2: 47327531C/T
5-2: 47328642C/T
5-2: 47579276T/G
5-2: 47604891T/A
5-2: 47662462T/A
5-2: 47702278G/A
5-2: 47712915T/C
5-2: 47740588A/G
5-2: 47746971C/G
5-2: 47801335C/T
5-2: 47830115A/C
5-2: 47886854G/T
5-2: 47891911G/A
5-2: 47891957T/A
5-2: 47891984A/T
5-2: 47947789G/T
5-2: 47963460C/A
5-2: 48016129G/A
5-2: 48056702T/C
5-2: 48195866T/A
5-2: 48257431G/A
5-2: 48320202A/G
5-2: 48366875G/T
5-2: 48407335G/T
5-2: 48437105G/A
5-2: 48506364T/A
5-2: 48627165G/A
5-2: 48646602C/G
5-2: 48659591C/G
5-2: 48776928C/T
5-2: 48782703C/A
5-2: 48834466C/A
5-2: 48969376A/C
5-2: 49027169T/A
5-2: 49076392T/A
5-2: 49138783A/G
5-2: 49140423T/G
5-2: 49140463C/A
5-2: 49142080C/A
5-2: 49142210C/T
5-2: 49217167C/T
5-2: 49217852T/G
5-2: 49317955C/T
5-2: 49367354T/C
5-2: 49433128C/T
5-2: 49529556G/T
5-2: 49590490A/G
5-2: 49621392A/G
5-2: 49659194T/G
5-2: 49691751A/C
5-2: 49784981C/T
5-2: 49866517T/C
5-2: 49912151A/C
5-2: 49924248A/G
5-2: 49949249T/C
5-2: 50010056T/C
5-2: 50067466G/T
5-2: 50095064A/C
5-2: 50146829C/T
5-2: 50185590G/T
5-2: 50230270C/T
5-2: 50334715C/G
5-2: 50349093C/A
5-2: 50359055A/T
5-2: 50373910G/T
5-2: 50414385C/T
5-2: 50416940T/C
5-2: 50429830C/T
5-2: 50440377T/C
5-2: 50464420A/C
5-2: 50509678G/A
5-2: 50539709T/A
5-2: 50572452A/C
5-2: 50631618C/A
5-2: 50652569G/A
5-2: 50652708G/A
5-2: 50721070T/C
5-2: 50802207T/A
5-2: 50873195C/T
5-2: 50890451T/C
5-2: 50894231G/T
5-2: 50907049A/G
5-2: 50954970C/T
5-2: 50961151C/T
5-2: 50961731A/C
5-2: 50962183C/T
5-2: 51000960T/C
5-2: 51027009A/G
5-2: 51118799G/A
5-2: 51129724T/C
5-2: 51171387C/T
5-2: 51219481C/T
5-2: 51245637G/A
5-2: 51328657C/T
5-2: 51340535G/A
5-2: 51355269C/A
5-2: 51381992G/A
5-2: 51439355C/A
5-2: 51501786G/A
5-2: 51557456T/G
5-2: 51567716A/C
5-2: 51588303A/T
5-2: 51637734A/T
5-2: 51652799G/T
5-2: 51657337A/C
5-2: 51663805A/C
5-2: 51679980T/C
5-2: 51687745A/G
5-2: 51688293C/T
5-2: 51688879C/A
5-2: 51690181C/A
5-2: 51690217C/G
5-2: 51708503A/C
5-2: 51730104A/T
5-2: 51790729T/C
5-2: 51819710G/A
5-2: 51875598A/G
5-2: 51940958G/T
5-2: 51944754A/G
5-2: 51992146T/C
5-2: 52004465A/G
5-2: 52011349C/T
5-2: 52011372G/C
5-2: 52083273T/C
5-2: 52114043C/T
5-2: 52167569G/A
5-2: 52222221G/A
5-2: 52295450C/T
5-2: 52311810C/T
5-2: 52333847G/A
5-2: 52365603G/A
5-2: 52395718G/T
5-2: 52427191G/A
5-2: 52439275C/T
5-2: 52469874G/A
5-2: 52500183G/A
5-2: 52531882A/G
5-2: 52549357G/C
5-2: 52563289A/C
5-2: 52584752C/T
5-2: 52627301T/A
5-2: 52663000T/C
5-2: 52692994C/A
5-2: 52703551C/T
5-2: 52735611C/T
5-2: 52762048T/G
5-2: 52763395T/C
5-2: 52763628A/G
5-2: 52777195A/C
5-2: 52802815C/T
5-2: 52822266A/T
5-2: 52835617T/C
5-2: 52858862A/T
5-2: 52871220C/G
5-2: 52906685T/G
5-2: 52928280T/C
5-2: 52971356G/T
5-2: 52983098G/A
5-2: 53011820T/C
5-2: 53060145C/T
5-2: 53080689C/T
5-2: 53103141C/T
5-2: 53135104T/A
5-2: 53231072A/T
5-2: 53242337C/G
5-2: 53288710T/C
5-2: 53299545G/A
5-2: 53374313C/T
5-2: 53400771A/C
5-2: 53427373T/A
5-2: 53489791C/T
5-2: 53540162T/C
5-2: 53542789G/A
5-2: 53550988C/T
5-2: 53573740A/C
5-2: 53596747T/C
5-2: 53616232C/G
5-2: 53631383T/C
5-2: 53683563T/G
5-2: 53723327T/C
5-2: 53735327G/T
5-2: 53766106G/A
5-2: 53804185C/T
5-2: 53812214A/T
5-2: 53853495G/A
5-2: 53855092G/C
5-2: 53903625A/G
5-2: 53928034T/C
5-2: 53949475C/A
5-2: 53971347A/C
5-2: 53988555A/C
5-2: 54019713T/C
5-2: 54226584T/A
5-2: 54239201G/A
5-2: 54427678C/T
5-2: 54485884C/A
5-2: 54542503C/T
5-2: 54571678A/G
5-2: 54601781C/A
5-2: 54869592T/A
5-2: 54905435A/T
5-2: 54915572T/C
5-2: 54954496G/T
5-2: 55018762C/G
5-2: 55057981T/G
5-2: 55149103C/T
5-2: 55215163G/T
5-2: 55279329C/T
5-2: 55327913C/A
5-2: 55417915G/A
5-2: 55519147G/A
5-2: 55521852A/G
5-2: 55548151G/A
5-2: 55588927G/A
5-2: 55600920A/G
5-2: 55640787C/T
5-2: 55652205T/G
5-2: 55696777T/C
5-2: 55698002G/A
5-2: 55723452A/G
5-2: 55765589C/T
5-2: 55815327A/T
5-2: 55867895G/T
5-2: 55919119A/G
5-2: 55949534T/C
5-2: 56032807T/C
5-2: 56067814C/T
5-2: 56097176T/G
5-2: 56142014C/G
5-2: 56204283A/C
5-2: 56261483A/T
5-2: 56287246C/A
5-2: 56322654C/G
5-2: 56346301T/C
5-2: 56429162G/A
5-2: 56440720T/A
5-2: 56465371G/A
5-2: 56472154A/G
5-2: 56500391T/C
5-2: 56514052C/T
5-2: 56543827T/C
5-2: 56616292G/A
5-2: 56634598T/C
5-2: 56645442C/A
5-2: 56655617T/C
5-2: 56672602T/C
5-2: 56673825T/C
5-2: 56675090G/A
5-2: 56687283A/C
5-2: 56691550A/T
5-2: 56706087C/T
5-2: 56706597C/G
5-2: 56706634T/A
5-2: 56732320G/C
5-2: 56773814C/T
5-2: 56789723A/G
5-2: 56808290A/G
5-2: 56844313G/A
5-2: 56861456A/T
5-2: 56885632G/C
5-2: 56908586C/A
5-2: 56925421T/G
5-2: 56926678C/T
5-2: 56936936G/T
5-2: 56953126A/G
5-2: 57008757C/T
5-2: 57051829T/C
5-2: 57095405G/A
5-2: 57164557G/C
5-2: 57284982T/A
5-2: 57354716A/G
5-2: 57431519A/G
5-2: 57565852G/A
5-2: 57619774G/C
5-2: 57646184T/C
5-2: 57782769C/A
5-2: 57833589T/A
5-2: 57897689A/C
5-2: 57941535C/T
5-2: 57979752A/C
5-2: 57979909G/T
5-2: 58259256C/A
5-2: 58271375G/A
5-2: 58301313T/C
5-2: 58311828T/G
5-2: 58373211T/A
5-2: 58418038C/T
5-2: 58419565C/T
5-2: 58441248A/T
5-2: 58533760T/C
5-2: 58560142G/T
5-2: 58644477T/C
5-2: 58674570C/A
5-2: 58751599C/T
5-2: 58804560C/T
5-2: 58886878T/C
5-2: 58900992G/A
5-2: 58994648T/C
5-2: 59026240A/G
5-2: 59093456G/T
5-2: 59162538A/G
5-2: 59216772C/G
5-2: 59362618T/C
5-2: 59444911C/A
5-2: 59570937C/T
5-2: 59581113T/C
5-2: 59600907G/C
5-2: 59612128G/T
5-2: 59642860T/C
5-2: 59653003C/G
5-2: 59657338C/T
5-2: 59734013A/G
5-2: 59783490C/G
5-2: 59826568G/A
5-2: 59841760G/T
5-2: 59923503C/A
5-2: 59973694T/C
5-2: 60038241A/C
5-2: 60073235C/T
5-2: 60101339T/C
5-2: 60142775A/G
5-2: 60233668T/C
5-2: 60262622T/C
5-2: 60275138A/C
5-2: 60277211G/A
5-2: 60362428T/C
5-2: 60389794T/G
5-2: 60427964C/T
5-2: 60439216C/T
5-2: 60477223T/C
5-2: 60602568C/A
5-2: 60614153A/G
5-2: 60621130C/G
5-2: 60625720C/A
5-2: 60786549G/A
5-2: 60952528A/G
5-2: 61036862G/A
5-2: 61111097A/G
5-2: 61279678C/T
5-2: 61372760G/A
5-2: 61375088G/A
5-2: 61438943A/G
5-2: 61468264C/A
5-2: 61493677C/T
5-2: 61674864C/T
5-2: 61773130T/G
5-2: 61825120T/C
5-2: 61851179T/A
5-2: 61974250G/T
5-2: 61989071T/C
5-2: 62103062A/T
5-2: 62128802A/G
5-2: 62138954A/G
5-2: 62220166G/A
5-2: 62279557T/C
5-2: 62316951A/T
5-2: 62328226C/T
5-2: 62374064C/G
5-2: 62428964T/C
5-2: 62479577C/T
5-2: 62603321G/A
5-2: 62613607G/A
5-2: 62645134T/G
5-2: 62671992A/G
5-2: 62728385T/C
5-2: 62806534A/C
5-2: 62912778A/G
5-2: 62962421T/C
5-2: 63025588T/G
5-2: 63174949C/T
5-2: 63255322T/A
5-2: 63340799G/A
5-2: 63409715C/T
5-2: 63461305C/T
5-2: 63505589G/A
5-2: 63587435C/G
5-2: 63608406G/A
5-2: 63716549C/T
5-2: 63717795G/A
5-2: 63768875G/A
5-2: 63796259C/A
5-2: 63842730A/C
5-2: 63898992G/A
5-2: 63930593T/C
5-2: 63977449A/G
5-2: 63996968G/C
5-2: 64089024T/A
5-2: 64123213A/T
5-2: 64207768A/G
5-2: 64251038G/A
5-2: 64277816T/G
5-2: 64335323C/A
5-2: 64338335T/C
5-2: 64365870C/A
5-2: 64413359T/C
5-2: 64435633G/T
5-2: 64463362T/C
5-2: 64475546C/A
5-2: 64497357C/T
5-2: 64504386G/T
5-2: 64544232T/G
5-2: 64544507A/G
5-2: 64551796T/C
5-2: 64552372A/T
5-2: 64598587G/A
5-2: 64646376C/T
5-2: 64663797C/A
5-2: 64734959A/C
5-2: 64770341G/A
5-2: 64916964C/T
5-2: 65008485T/C
5-2: 65058273A/T
5-2: 65074715G/A
5-2: 65165338G/A
5-2: 65206065G/A
5-2: 65211638T/A
5-2: 65229696C/G
5-2: 65330443A/C
5-2: 65346200T/C
5-2: 65347360A/C
5-2: 65359880A/G
5-2: 65369617C/T
5-2: 65371295C/T
5-2: 65416336T/G
5-2: 65419259G/C
5-2: 65422838G/A
5-2: 65423090T/C
5-2: 65423616A/G
5-2: 65423705T/G
5-2: 65435128C/T
5-2: 65454239T/G
5-2: 65485715G/A
5-2: 65498888G/A
5-2: 65531912T/C
5-2: 65594435A/G
5-2: 65718823C/A
5-2: 65732641A/T
5-2: 65751621A/G
5-2: 65777038C/T
5-2: 65787405C/T
5-2: 65802904G/T
5-2: 65891298T/A
5-2: 65905288A/G
5-2: 65914514A/G
5-2: 65942770G/A
5-2: 65952498A/G
5-2: 65953012C/T
5-2: 65954124G/C
5-2: 65954522A/G
5-2: 65962429T/A
5-2: 65962552A/G
5-2: 65964138C/T
5-2: 65966696G/C
5-2: 65968590A/T
5-2: 66008033A/G
5-2: 66047707C/A
5-2: 66112500T/C
5-2: 66191585C/A
5-2: 66198337T/G
5-2: 66221221A/T
5-2: 66231923C/T
5-2: 66253662G/A
5-2: 66264526C/T
5-2: 66340802C/T
5-2: 66433149A/G
5-2: 66507804T/G
5-2: 66617213T/C
5-2: 66628898A/G
5-2: 66670461G/A
5-2: 66671860T/A
5-2: 66724200G/A
5-2: 66724702C/G
5-2: 66724711G/A
5-2: 66753716G/C
5-2: 66771286A/G
5-2: 66827326A/T
5-2: 66874902A/G
5-2: 66907422C/T
5-2: 66949466T/G
5-2: 67040688G/A
5-2: 67051548A/G
5-2: 67071249T/G
5-2: 67151784T/C
5-2: 67199490A/G
5-2: 67233099C/T
5-2: 67244128A/G
5-2: 67257026T/G
5-2: 67267603T/G
5-2: 67280011T/G
5-2: 67293030A/T
5-2: 67342115C/T
5-2: 67470586T/A
5-2: 67514472G/C
5-2: 67581835G/A
5-2: 67641846G/A
5-2: 67655921T/G
5-2: 67675658T/G
5-2: 67728916C/T
5-2: 67781175T/A
5-2: 67832883G/C
5-2: 67873271T/G
5-2: 67949638C/A
5-2: 68006163G/T
5-2: 68098683C/T
5-2: 68175406T/C
5-2: 68190489G/C
5-2: 68207239A/G
5-2: 68217722T/A
5-2: 68218897C/T
5-2: 68237801G/A
5-2: 68266507G/A
5-2: 68304306A/C
5-2: 68409155G/A
5-2: 68488704C/G
5-2: 68537418G/A
5-2: 68568590T/C
5-2: 68579740T/C
5-2: 68602244C/T
5-2: 68617780T/C
5-2: 68633135G/C
5-2: 68645331A/G
5-2: 68684286T/G
5-2: 68775641A/G
5-2: 68795952C/T
5-2: 68808931A/G
5-2: 68821316A/G
5-2: 68845823G/C
5-2: 68856073T/C
5-2: 68882019T/C
5-2: 68912608T/G
5-2: 68943746T/C
5-2: 68969512A/G
5-2: 68996796A/G
5-2: 69030724A/G
5-2: 69031234G/A
5-2: 69036310G/A
5-2: 69040798G/T
5-2: 69051730T/C
5-2: 69056031A/G
5-2: 69064137A/G
5-2: 69092467T/C
5-2: 69128725A/G
5-2: 69144410T/A
5-2: 69163309G/A
5-2: 69184781A/T
5-2: 69194988G/A
5-2: 69239181C/T
5-2: 69284196A/G
5-2: 69331865C/T
5-2: 69360533T/G
5-2: 69406512C/T
5-2: 69425236G/A
5-2: 69430454A/C
5-2: 69431531C/T
5-2: 69435814T/C
5-2: 69436544C/A
5-2: 69466307C/.
5-2: 69480633C/T
5-2: 69516202C/G
5-2: 69526783G/A
5-2: 69544831C/G
5-2: 69584242T/A
5-2: 69598355G/A
5-2: 69666005T/C
5-2: 69686919C/T
5-2: 69722753A/T
5-2: 69744397A/T
5-2: 69846789T/A
5-2: 69857208A/T
5-2: 69865837G/A
5-2: 69867690T/C
5-2: 69906690A/G
5-2: 69908526C/A
5-2: 69936657G/A
5-2: 69995637C/T
5-2: 70019450A/T
5-2: 70038989G/A
5-2: 70195375T/C
5-2: 70292647C/G
5-2: 70349800C/T
5-2: 70360369A/C
5-2: 70445068G/T
5-2: 70456179G/A
5-2: 70496640A/T
5-2: 70557546C/T
5-2: 70700524T/G
5-2: 70716735C/T
5-2: 70768120C/T
5-2: 70820327G/C
5-2: 70906816T/G
5-2: 71004348C/T
5-2: 71032596T/A
5-2: 71092025C/T
5-2: 71147245C/T
5-2: 71161334A/G
5-2: 71194256A/T
5-2: 71328231G/A
5-2: 71480914A/G
5-2: 71588532A/G
5-2: 71631718T/G
5-2: 71704138T/C
5-2: 71726709G/A
5-2: 71737318A/T
5-2: 71769960G/A
5-2: 71775135A/G
5-2: 71784538A/T
5-2: 71797718C/A
5-2: 71830928A/G
5-2: 71861513A/G
5-2: 71904206G/T
5-2: 71917660C/A
5-2: 71929777G/T
5-2: 71992598C/G
5-2: 72126534G/A
5-2: 72175411C/T
5-2: 72203763A/G
5-2: 72252085A/T
5-2: 72301483T/C
5-2: 72353202G/A
5-2: 72366348C/T
5-2: 72420055G/A
5-2: 72475982G/T
5-2: 72501693T/G
5-2: 72526915C/G
5-2: 72590449G/A
5-2: 72640591T/G
5-2: 72684966A/G
5-2: 72783832A/G
5-2: 72851319G/A
5-2: 72949427C/G
5-2: 72951202A/T
5-2: 72952908T/C
5-2: 72953417G/A
5-2: 72953548A/G
5-2: 73008013T/C
5-2: 73072227T/A
5-2: 73157482A/G
5-2: 73212726C/T
5-2: 73283823A/T
5-2: 73295524T/A
5-2: 73373103A/C
5-2: 73419352A/G
5-2: 73448865C/T
5-2: 73458254C/T
5-2: 73474573G/A
5-2: 73505544G/A
5-2: 73536178T/C
5-2: 73567280A/T
5-2: 73594058C/T
5-2: 73641803A/G
5-2: 73671928T/C
5-2: 73692561G/A
5-2: 73734503G/C
5-2: 73795346A/G
5-2: 73886866T/C
5-2: 73941613C/T
5-2: 74085694T/C
5-2: 74120345T/A
5-2: 74138744G/A
5-2: 74182017G/A
5-2: 74254374G/A
5-2: 74337502T/C
5-2: 74436073A/G
5-2: 74473266G/A
5-2: 74498749C/T
5-2: 74600977A/G
5-2: 74753351T/C
5-2: 74764397A/C
5-2: 74833833G/A
5-2: 74880062A/C
5-2: 74914900G/T
5-2: 75186776T/C
5-2: 75199271C/T
5-2: 75282557G/A
5-2: 75314141T/C
5-2: 75341832G/A
5-2: 75599008T/A
5-2: 75844960G/C
5-2: 76036251G/A
5-2: 76048687T/C
5-2: 76150962G/T
5-2: 76180049A/T
5-2: 76300089G/A
5-2: 76360023A/G
5-2: 76580344T/A
5-2: 76625978G/T
5-2: 76776626G/A
5-2: 76788557G/A
5-2: 76937756C/T
5-2: 77082377T/A
5-2: 77104984A/T
5-2: 77123516G/A
5-2: 77334218G/A
5-2: 77377554A/T
5-2: 77482908T/A
5-2: 77716475T/C
5-2: 78180194T/A
5-2: 78454360G/A
5-2: 78647504A/G
5-2: 78859911G/A
5-2: 78924357C/T
5-2: 78954220A/T
5-3: 81657C/T
5-3: 150457G/A
5-3: 330130A/G
5-3: 345581G/T
5-3: 401395G/C
5-3: 430628A/T
5-3: 493349T/A
5-3: 550531T/A
5-3: 694440C/T
5-3: 807085C/T
5-3: 1034290C/T
5-3: 1264384A/T
5-3: 1502102C/T
5-3: 1517220C/T
5-3: 1545288T/A
5-3: 1670147C/T
5-3: 1692345A/C
5-3: 1839204G/C
5-3: 1864246G/T
5-3: 2014448A/G
5-3: 2029769G/A
5-3: 2053863T/A
5-3: 2071425C/T
5-3: 2133315A/G
5-3: 2156578A/G
5-3: 2173611G/A
5-3: 2371636G/A
5-3: 2542189A/G
5-3: 2649142A/G
5-3: 2695990A/G
5-3: 2823391G/T
5-3: 2914500C/T
5-3: 2934396A/G
5-3: 3074254C/A
5-3: 3322980T/C
5-3: 3333600C/T
5-3: 3344547G/C
5-3: 3358246G/T
5-3: 3465869G/A
5-3: 3611684A/G
5-3: 3689640G/A
5-3: 3700150A/C
5-3: 3913191T/G
5-3: 3914651C/T
5-3: 4015272A/G
5-3: 4059130T/C
5-3: 4218971A/G
5-3: 4229170C/T
5-3: 4301244T/G
5-3: 4432182T/C
5-3: 4465043C/A
5-3: 4508425A/T
5-3: 4619852A/T
5-3: 4621041G/A
5-3: 4625215A/G
5-3: 4626661T/C
5-3: 4631404C/A
5-3: 4643096A/C
5-3: 4754497C/T
5-3: 4756332T/C
5-3: 4793610C/T
5-3: 4836028G/T
5-3: 4871832G/A
5-3: 4967090A/C
5-3: 5004887A/T
5-3: 5096959G/A
5-3: 5257009C/T
5-3: 5275804C/T
5-3: 5370265C/T
5-3: 5631429G/C
5-3: 5826875C/T
5-3: 5832247A/T
5-3: 5866207G/A
5-3: 5884796C/T
5-3: 5922377A/C
5-3: 5948483G/T
5-3: 5987475G/T
5-3: 6013281G/A
5-3: 6025989A/T
5-3: 6079574C/A
5-3: 6102954G/A
5-3: 6231691A/T
5-3: 6326463A/T
5-3: 6450321G/A
5-3: 6468679A/G
5-3: 6606127G/A
5-3: 6649841T/G
5-3: 6686722C/A
5-3: 6722718G/T
5-3: 6774948A/G
5-3: 6800506G/A
5-3: 6879176C/T
5-3: 6911110T/A
5-3: 6922198A/T
5-3: 7182862A/G
5-3: 7262600T/G
5-3: 7269231A/C
5-3: 7282480C/T
5-3: 7310551A/G
5-3: 7322913G/C
5-3: 7333859A/G
5-3: 7346089C/T
5-3: 7396182G/C
5-3: 7416526G/C
5-3: 7430774T/C
5-3: 7450262T/A
5-3: 7489883G/A
5-3: 7545529A/G
5-3: 7872153T/A
5-3: 7872175T/C
5-3: 7872984C/T
5-3: 7994212A/C
5-3: 8111603C/T
5-3: 8130287C/T
5-3: 8231565A/G
5-3: 8366052T/A
5-3: 8366235A/C
5-3: 8376326C/T
5-3: 8402709C/T
5-3: 8414680T/A
5-3: 8458224G/A
5-3: 8490698T/C
5-3: 8564149T/G
5-3: 8599044T/C
5-3: 8667262C/T
5-3: 8713328A/G
5-3: 8803477T/C
5-3: 8850235G/T
5-3: 8925935C/A
5-3: 8945088G/A
5-3: 8969142C/T
5-3: 8995752C/T
5-3: 9016219T/C
5-3: 9129492T/C
5-3: 9158101A/G
5-3: 9172052T/C
5-3: 9172123G/A
5-3: 9178892G/T
5-3: 9214120T/A
5-3: 9286045A/G
5-3: 9302334A/G
5-3: 9340902A/G
5-3: 9366431G/A
5-3: 9406312A/G
5-3: 9456355T/C
5-3: 9624017G/C
5-3: 9653703C/T
5-3: 9708693C/T
5-3: 9808290T/G
5-3: 9894154A/G
5-3: 9930076G/A
5-3: 9961971T/C
5-3: 10040481A/C
5-3: 10091048C/A
5-3: 10118538C/T
5-3: 10154558G/C
5-3: 10194958C/T
5-3: 10197450C/A
5-3: 10285097T/C
5-3: 10351502C/T
5-3: 10353954A/T
5-3: 10379150T/C
5-3: 10395209T/C
5-3: 10407634T/A
5-3: 10519312T/C
5-3: 10567154G/T
5-3: 10598567A/G
5-3: 10614847C/T
5-3: 10646803T/A
5-3: 10725503G/A
5-3: 10773479A/T
5-3: 10828891A/T
5-3: 10861885T/C
5-3: 10985841C/T
5-3: 11012964T/C
5-3: 11050104T/G
5-3: 11108929T/C
5-3: 11142271A/C
5-3: 11182349A/G
5-3: 11220092T/C
5-3: 11258823A/T
5-3: 11328639C/T
5-3: 11345493G/C
5-3: 11348115T/C
5-3: 11377521A/G
5-3: 11389958T/C
5-3: 11481599A/G
5-3: 11670803T/C
5-3: 11687864G/T
5-3: 11828312T/C
5-3: 11843205A/T
5-3: 11865365G/T
5-3: 11878380A/G
5-3: 11968913G/T
5-3: 11990531G/A
5-3: 11990842A/T
5-3: 12014666C/A
5-3: 12033328T/A
5-3: 12107949T/C
5-3: 12146327G/A
5-3: 12195363G/A
5-3: 12239022T/A
5-3: 12311420T/C
5-3: 12352366A/G
5-3: 12502046G/T
5-3: 12530703A/C
5-3: 12569849C/T
5-3: 12636446G/A
5-3: 12690301A/T
5-3: 12727402G/A
5-3: 12766002A/C
5-3: 12807775A/T
5-3: 12857287T/C
5-3: 12894958G/A
5-3: 12966827G/A
5-3: 13002373T/A
5-3: 13002499T/G
5-3: 13236522C/T
5-3: 13303141G/A
5-3: 13343890G/A
5-3: 13360564C/T
5-3: 13381845A/G
5-3: 13437333G/A
5-3: 13437380C/A
5-3: 13450873A/G
5-3: 13467126T/G
5-3: 13604083A/C
5-3: 13782340C/T
5-3: 13792356C/A
5-3: 13846862G/A
5-3: 13856816G/A
5-3: 13886093A/T
5-3: 13933815G/T
5-3: 13962072A/C
5-3: 13995575A/T
5-3: 14058441G/T
5-3: 14121167C/T
5-3: 14147033G/A
5-3: 14178135C/T
5-3: 14303219C/T
5-3: 14318642C/A
5-3: 14351048G/A
5-3: 14384350G/T
5-3: 14416684C/T
5-3: 14478736C/T
5-3: 14525956A/G
5-3: 14557732C/T
5-3: 14583219G/T
5-3: 14646739A/G
5-3: 14663128A/T
5-3: 14677786C/G
5-3: 14692586C/T
5-3: 14705101G/C
5-3: 14734520T/C
5-3: 14784850C/T
5-3: 14844812C/A
5-3: 14870834C/A
5-3: 14897116T/C
5-3: 14926108A/T
5-3: 14948758C/T
5-3: 14962888A/G
5-3: 15102006A/C
5-3: 15234657A/G
5-3: 15351333T/C
5-3: 15456962C/T
5-3: 15574572C/A
5-3: 15591347C/T
5-3: 15604051C/A
5-3: 15615857A/C
5-3: 15644644C/T
5-3: 15735141A/T
5-3: 15816128G/A
5-3: 15819591A/C
5-3: 15834985C/T
5-3: 15860951A/G
5-3: 15889339G/T
5-3: 15932640G/A
5-3: 15982753A/T
5-3: 16024561G/T
5-3: 16070695T/A
5-3: 16100995G/A
5-3: 16161948C/T
5-3: 16161962A/G
5-3: 16170364C/T
5-3: 16207571T/.
5-3: 16208887A/G
5-3: 16224485A/G
5-3: 16224930C/T
5-3: 16242321C/T
5-3: 16253236T/C
5-3: 16261311G/A
5-3: 16266585T/G
5-3: 16277331T/A
5-3: 16288639G/A
5-3: 16289244G/A
5-3: 16292873A/G
5-3: 16292906C/T
5-3: 16294737C/T
5-3: 16305016A/T
5-3: 16330264C/A
5-3: 16334393A/G
5-3: 16365819A/G
5-3: 16386983A/G
5-3: 16407723T/C
5-3: 16424143G/A
5-3: 16471886G/A
5-3: 16499884A/T
5-3: 16514059T/C
5-3: 16527680G/T
5-3: 16537449A/G
5-3: 16577354T/A
5-3: 16594454G/A
5-3: 16612287C/G
5-3: 16632750C/A
5-3: 16637423A/G
5-3: 16742113A/G
5-3: 16760024A/G
5-3: 16780820A/G
5-3: 16802806A/G
5-3: 16803501C/T
5-3: 16803512G/A
5-3: 16803582T/G
5-3: 16803602C/G
5-3: 16803641G/A
5-3: 16808846T/A
5-3: 16829354T/C
5-3: 16832590A/C
5-3: 16845720A/G
5-3: 16899130A/T
5-3: 16917069A/T
5-3: 16931020C/G
5-3: 16960613A/T
5-3: 16982474G/A
5-3: 16994066C/T
5-3: 17084196T/A
5-3: 17096156T/G
5-3: 17096350A/C
5-3: 17098596C/T
5-3: 17123206T/G
5-3: 17136646C/T
5-3: 17161442G/A
5-3: 17181194A/C
5-3: 17181775G/A
5-3: 17240870A/G
5-3: 17297735A/T
5-3: 17310777T/C
5-3: 17329421G/T
5-3: 17350608C/T
5-3: 17391001G/C
5-3: 17438561C/G
5-3: 17441933C/T
5-3: 17442271G/A
5-3: 17462076G/C
5-3: 17468557C/A
5-3: 17468913A/T
5-3: 17469602A/T
5-3: 17472024A/G
5-3: 17473279A/G
5-3: 17483617C/T
5-3: 17493952G/A
5-3: 17539941G/T
5-3: 17590248C/G
5-3: 17606684T/C
5-3: 17622322C/T
5-3: 17637624C/A
5-3: 17667399C/T
5-3: 17677839A/G
5-3: 17692401G/C
5-3: 17716485A/C
5-3: 17722539C/T
5-3: 17743361G/A
5-3: 17772501T/C
5-3: 17791335G/A
5-3: 17835341C/G
5-3: 17923180A/G
5-3: 18019287T/C
5-3: 18029462T/C
5-3: 18031126G/C
5-3: 18041412T/C
5-3: 18137038T/C
5-3: 18147932G/A
5-3: 18191713C/A
5-3: 18211198A/G
5-3: 18265538A/T
5-3: 18336876C/T
5-3: 18347210C/G
5-3: 18361827G/T
5-3: 18385555C/T
5-3: 18407980A/C
5-3: 18459698C/A
5-3: 18547065T/A
5-3: 18600536C/T
5-3: 18617232G/A
5-3: 18629489G/C
5-3: 18724173G/T
5-3: 18781613C/T
5-3: 18816800C/T
5-3: 18838073A/G
5-3: 18860422A/T
5-3: 18888405A/G
5-3: 19011009C/G
5-3: 19012092T/G
5-3: 19083109C/T
5-3: 19116618G/A
5-3: 19164493T/C
5-3: 19180194T/C
5-3: 19249482G/A
5-3: 19405365C/T
5-3: 19431651G/A
5-3: 19467751A/C
5-3: 19501335C/T
5-3: 19558003C/G
5-3: 19670334C/T
5-3: 19683102C/A
5-3: 19687565A/G
5-3: 19699110G/A
5-3: 19866005T/C
5-3: 19894593C/G
5-3: 19908442T/G
5-3: 20048242G/A
5-3: 20194171G/A
5-3: 20241018C/T
5-3: 20344886T/G
5-3: 20418540T/C
5-3: 20579926A/G
5-3: 20641728A/G
5-3: 20697514A/T
5-3: 20723084C/T
5-3: 20773975A/G
5-3: 20829449T/C
5-3: 20905863A/T
5-3: 20964541C/T
5-3: 21022355T/C
5-3: 21080430A/G
5-3: 21166468G/T
5-3: 21200703T/A
5-3: 21202119C/T
5-3: 21251767G/C
5-3: 21267146T/A
5-3: 21296726G/A
5-3: 21318535A/C
5-3: 21369683T/C
5-3: 21390022A/G
5-3: 21495467A/G
5-3: 21496850T/G
5-3: 21505922A/G
5-3: 21526206A/C
5-3: 21537552A/G
5-3: 21558565G/A
5-3: 21570121C/A
5-3: 21590439G/T
5-3: 21631556G/A
5-3: 21635887G/A
5-3: 21642748A/T
5-3: 21665574T/A
5-3: 21680371G/A
5-3: 21712923A/T
5-3: 21723836C/A
5-3: 21737026G/C
5-3: 21749405T/C
5-3: 21785956A/G
5-3: 21800180C/T
5-3: 21833876C/A
5-3: 21861810A/G
5-3: 21877303G/T
5-3: 21893733A/T
5-3: 21898059A/G
5-3: 21906436A/T
5-3: 21953671G/C
5-3: 21997316T/C
5-3: 22019149T/C
5-3: 22046860A/G
5-3: 22061303T/G
5-3: 22085290A/C
5-3: 22106371C/T
5-3: 22135213A/G
5-3: 22177720G/C
5-3: 22200063A/G
5-3: 22218303G/A
5-3: 22228835A/T
5-3: 22232884C/T
5-3: 22256982G/A
5-3: 22282398A/G
5-3: 22314392G/C
5-3: 22333261C/T
5-3: 22361603G/A
5-3: 22436748A/G
5-3: 22540559T/C
5-3: 22549003A/G
5-3: 22577393G/A
5-3: 22589992A/T
5-3: 22639045C/T
5-3: 22676449G/A
5-3: 22804470A/G
5-3: 22852101T/C
5-3: 22874700G/A
5-3: 22920013T/C
5-3: 23048401G/C
5-3: 23147419T/C
5-3: 23165508A/T
5-3: 23212873C/T
5-3: 23229507A/T
5-3: 23319169A/T
5-3: 23333148A/T
5-3: 23398981T/C
5-3: 23443588A/T
5-3: 23459731T/C
5-3: 23501295T/C
5-3: 23553570G/A
5-3: 23566692C/T
5-3: 23575926G/A
5-3: 23581370T/G
5-3: 23593334A/G
5-3: 23614315G/T
5-3: 23618035C/T
5-3: 23624563G/A
5-3: 23647430T/C
5-3: 23658205A/G
5-3: 23661453T/C
5-3: 23665405G/A
5-3: 23680016G/A
5-3: 23724745C/A
5-3: 23734761C/A
5-3: 23776371C/T
5-3: 23845361T/G
5-3: 24045509A/T
5-3: 24181099A/G
5-3: 24565475A/G
5-3: 24936430T/G
5-3: 25306010C/G
5-3: 25443573G/C
5-3: 26304641G/T
5-3: 26361251T/G
5-3: 26431511C/A
5-3: 26443790A/C
5-3: 26448548C/T
5-3: 26683887A/G
5-3: 26694819A/T
5-3: 26847679C/T
5-3: 26940539A/G
5-3: 27194034C/T
5-3: 27239378A/G
5-3: 27314358T/C
5-3: 27326993G/T
5-3: 27345576A/G
5-3: 27346421T/A
5-3: 27346519A/G
5-3: 27370069G/T
5-3: 27655420T/C
5-3: 27669776C/T
5-3: 27691624G/A
5-3: 27705331C/T
5-3: 27739810T/G
5-3: 27806561G/A
5-3: 27852103G/A
5-3: 27866564G/A
5-3: 27971196C/G
5-3: 27972618G/A
5-3: 28176815T/A
5-3: 28209640C/T
5-3: 28237779G/A
5-3: 28479658G/T
5-3: 28544331C/T
5-3: 28589395G/C
5-3: 28783789C/T
5-3: 28864378T/G
5-3: 28919642T/C
5-3: 28919705G/A
5-3: 29002691A/G
5-3: 29243622T/C
5-3: 29261280G/T
5-3: 29261555T/.
5-3: 29261699A/.
5-3: 29261703A/.
5-3: 29261711C/.
5-3: 29261720T/.
5-3: 29261726A/.
5-3: 29261759A/.
5-3: 29261763G/.
5-3: 29261999A/G
5-3: 29262674G/A
5-3: 29262698T/C
5-3: 29263370A/T
5-3: 29263472T/G
5-3: 29263495C/.
5-3: 29263506C/.
5-3: 29263555C/.
5-3: 29331843G/A
5-3: 29399361G/T
5-3: 29448561T/C
5-3: 29747945A/G
5-3: 29784581G/C
5-3: 29989747C/A
5-3: 30068995C/T
5-3: 30113019A/C
5-3: 30160940C/T
5-3: 30189014C/G
5-3: 30471846G/A
5-3: 30482572A/T
5-3: 30515410C/A
5-3: 30547577T/C
5-3: 30610750T/A
5-3: 30682128C/A
5-3: 30704258T/C
5-3: 30865742G/A
5-3: 30965725T/A
5-3: 30969415T/C
5-3: 30989190A/T
5-3: 31000412A/G
5-3: 31037978T/G
5-3: 31102994C/T
5-3: 31201615C/T
5-3: 31218391A/G
5-3: 31315858C/T
5-3: 31321964A/T
5-3: 31353371G/A
5-3: 31403929A/G
5-3: 31430885A/T
5-3: 31458235G/A
5-3: 31487059T/A
5-3: 31532546T/C
5-3: 31648544T/C
5-3: 31652648T/C
5-3: 31701200G/T
5-3: 31714145A/G
5-3: 31724352C/T
5-3: 31741816A/G
5-3: 31753366T/C
5-3: 31817799C/T
5-3: 31901372A/C
5-3: 31907225T/G
5-3: 31936663T/C
5-3: 32041506T/C
5-3: 32069595A/C
5-3: 32089079A/C
5-3: 32108688C/T
5-3: 32144099C/T
5-3: 32195830T/C
5-3: 32228630T/C
5-3: 32297345G/A
5-3: 32310784T/C
5-3: 32330903C/T
5-3: 32343700A/G
5-3: 32424154A/C
5-3: 32496022T/C
5-3: 32567583A/G
5-3: 32595748G/A
5-3: 32607692T/C
5-3: 32618989A/T
5-3: 32650719A/G
5-3: 32727339G/C
5-3: 32786216A/G
5-3: 32796329G/T
5-3: 32825064T/A
5-3: 32865044C/A
5-3: 32893157T/A
5-3: 32932699G/A
5-3: 32984933C/A
5-3: 33054553C/T
5-3: 33109396C/T
5-3: 33166728A/C
5-3: 33263918T/C
5-3: 33336022C/T
5-3: 33340480A/G
5-3: 33348223G/A
5-3: 33374212G/A
5-3: 33443074A/T
5-3: 33456628C/T
5-3: 33466906C/T
5-3: 33552289G/A
5-3: 33563148T/C
5-3: 33601432T/C
5-3: 33656535A/G
5-3: 33879022G/A
5-3: 33974793G/A
5-3: 34019714T/C
5-3: 34093491C/T
5-3: 34144771G/A
5-3: 34219816T/C
5-3: 34247413A/T
5-3: 34257493A/T
5-3: 34282121G/A
5-3: 34321579G/T
5-3: 34358272C/T
5-3: 34409891T/C
5-3: 34422143C/A
5-3: 34437598C/T
5-3: 34449320A/C
5-3: 34461586A/G
5-3: 34464481C/T
5-3: 34469126G/T
5-3: 34498374A/T
5-3: 34524712C/T
5-3: 34565649G/A
5-3: 34600739C/T
5-3: 34632126C/T
5-3: 34688221T/C
5-3: 34743177G/T
5-3: 34767030A/G
5-3: 34778245G/A
5-3: 34790330G/T
5-3: 34793119C/T
5-3: 34802098A/G
5-3: 34814824A/G
5-3: 34827363T/A
5-3: 34841149G/T
5-3: 34887471A/C
5-3: 34901510A/T
5-3: 34931931C/T
5-3: 34934613G/T
5-3: 34979877A/G
5-3: 34979891T/A
5-3: 34990990T/C
5-3: 35127817A/C
5-3: 35135306G/A
5-3: 35214136A/G
5-3: 35248679C/A
5-3: 35291725C/T
5-3: 35305782A/G
5-3: 35328957G/A
5-3: 35421451G/A
5-3: 35445081G/T
5-3: 35512847G/A
5-3: 35560846C/T
5-3: 35598148A/T
5-3: 35718962C/T
5-3: 35755879C/A
5-3: 35766260T/C
5-3: 35821637A/G
5-3: 35901057A/G
5-3: 35956917T/C
5-3: 36034093C/T
5-3: 36150573T/C
5-3: 36209239A/C
5-3: 36261910G/A
5-3: 36296729C/T
5-3: 36298061C/A
5-3: 36307663A/T
5-3: 36418355T/A
5-3: 36477478G/T
5-3: 36495361C/T
5-3: 36524536C/T
5-3: 36596098T/C
5-3: 36606415G/A
5-3: 36683630C/T
5-3: 36693691T/C
5-3: 36798901G/T
5-3: 36831506T/C
5-3: 36910687T/G
5-3: 36925308G/A
5-3: 36935696A/T
5-3: 36945757T/C
5-3: 36973690T/C
5-3: 37014564A/C
5-3: 37042627A/T
5-3: 37090907T/C
5-3: 37110123T/C
5-3: 37141245C/A
5-3: 37145655G/A
5-3: 37163357T/A
5-3: 37196649A/C
5-3: 37210171C/T
5-3: 37244637T/C
5-3: 37298763A/G
5-3: 37333226A/T
5-3: 37367674A/G
5-3: 37449834G/A
5-3: 37487704G/A
5-3: 37536589C/G
5-3: 37587406G/A
5-3: 37675179C/A
5-3: 37754063G/T
5-3: 37811911C/T
5-3: 37816110A/T
5-3: 37858162G/C
5-3: 37873762G/T
5-3: 37885872A/T
5-3: 37897333G/A
5-3: 37931100G/T
5-3: 37941181A/G
5-3: 37943854G/C
5-3: 37960737G/A
5-3: 37989754A/T
5-3: 38019198G/A
5-3: 38050122T/G
5-3: 38060369A/C
5-3: 38093940G/C
5-3: 38104331C/T
5-3: 38136719G/T
5-3: 38228403T/C
5-3: 38335742A/G
5-3: 38477750T/C
5-3: 38540669C/A
5-3: 38595004G/T
5-3: 38603655A/G
5-3: 38646868A/C
5-3: 38677461T/C
5-3: 38862562G/C
5-3: 38972350T/G
5-3: 38987626A/C
5-3: 39040575G/C
5-3: 39051274T/C
5-3: 39051349T/C
5-3: 39051374T/A
5-3: 39051448C/T
5-3: 39051556A/T
5-3: 39051655T/C
5-3: 39051963C/T
5-3: 39051966T/C
5-3: 39051985T/C
5-3: 39052109A/G
5-3: 39052110A/T
5-3: 39052129A/C
5-3: 39052135C/T
5-3: 39052270A/G
5-3: 39084957T/C
5-3: 39119831A/G
5-3: 39246166G/A
5-3: 39253644C/A
5-3: 39322023G/A
5-3: 39340159T/C
5-3: 39462741G/A
5-3: 39483359G/C
5-3: 39609696C/A
5-3: 39628257T/C
5-3: 39670748C/G
5-3: 39723246C/A
5-3: 39778464T/A
5-3: 39789315C/T
5-3: 39812116G/A
5-3: 39882522C/A
5-3: 39886178T/A
5-3: 39887750T/C
5-3: 39925471A/T
5-3: 39964378C/T
5-3: 39976451G/A
5-3: 39978735T/C
5-3: 39978863T/C
5-3: 39978971T/C
5-3: 39979388A/C
5-3: 39979412A/G
5-3: 40039548C/T
5-3: 40059642A/C
5-3: 40130537T/G
5-3: 40170662T/C
5-3: 40183535A/G
5-3: 40310966T/A
5-3: 40342097C/G
5-3: 40447877T/C
5-3: 40465595T/C
5-3: 40505462C/G
5-3: 40534520T/G
5-3: 40546472A/G
5-3: 40547138A/T
5-3: 40547297G/A
5-3: 40587432C/A
5-3: 40588975G/T
5-3: 40725136G/T
5-3: 40777870A/G
5-3: 40810813G/C
5-3: 40900429G/A
5-3: 40989078G/T
5-3: 41017877A/G
5-3: 41046409T/G
5-3: 41102341G/T
5-3: 41150514C/T
5-3: 41205981A/G
5-3: 41240696T/A
5-3: 41241167G/A
5-3: 41324035G/C
5-3: 41353170A/G
5-3: 41374013A/G
5-3: 41394388T/A
5-3: 41409914C/G
5-3: 41547358A/T
5-3: 41633353C/T
5-3: 41635373A/G
5-3: 41651160C/T
5-3: 41656126C/T
5-3: 41723137T/C
5-3: 41738634A/T
5-3: 41757613G/A
5-3: 41794329C/T
5-3: 41829760C/T
5-3: 41874589A/C
5-3: 41928016A/C
5-3: 41985856G/A
5-3: 42070723C/T
5-3: 42133443C/A
5-3: 42147302C/T
5-3: 42177829A/T
5-3: 42194375C/T
5-3: 42204507G/A
5-3: 42224353A/G
5-3: 42287391T/G
5-3: 42334066A/G
5-3: 42364666T/C
5-3: 42398427C/T
5-3: 42425310T/C
5-3: 42475681C/T
5-3: 42598041G/A
5-3: 42609570G/A
5-3: 42634673C/T
5-3: 42664393C/T
5-3: 42723566G/A
5-3: 42756431T/C
5-3: 42797311T/A
5-3: 42892153A/T
5-3: 42940005C/T
5-3: 42982002G/T
5-3: 43013376C/T
5-3: 43097166A/G
5-3: 43101578G/T
5-3: 43113930C/T
5-3: 43126914T/C
5-3: 43170786T/A
5-3: 43291480G/A
5-3: 43338851T/A
5-3: 43380672T/C
5-3: 43381441A/G
5-3: 43401602A/G
5-3: 43401853A/G
5-3: 43404987T/G
5-3: 43406040G/A
5-3: 43429901C/T
5-3: 43460448T/C
5-3: 43460925G/A
5-3: 43484517T/G
5-3: 43507721G/A
5-3: 43534093T/G
5-3: 43559691C/T
5-3: 43565188C/T
5-3: 43572532C/A
5-3: 43574179G/A
5-3: 43601594A/T
5-3: 43635396C/T
5-3: 43662132G/A
5-3: 43712545T/C
5-3: 43729956C/T
5-3: 43755080G/A
5-3: 43786362T/C
5-3: 43844166G/T
5-3: 43937341T/C
5-3: 43948670T/A
5-3: 43976050T/C
5-3: 44039338C/T
5-3: 44081118G/A
5-3: 44121782T/C
5-3: 44174263G/A
5-3: 44311693G/T
5-3: 44324742C/T
5-3: 44337530C/T
5-3: 44375776G/A
5-3: 44409475C/T
5-3: 44442354A/G
5-3: 44490027A/C
5-3: 44518310C/T
5-3: 44566285C/T
5-3: 44603924G/T
5-3: 44662772C/G
5-3: 44675177A/G
5-3: 44692987T/C
5-3: 44695760C/T
5-3: 44696117C/T
5-3: 44705367C/G
5-3: 44746317C/G
5-3: 44804166G/C
5-3: 44821272T/A
5-3: 44839672C/T
5-3: 44872304A/T
5-3: 44882608A/T
5-3: 45041626G/A
5-3: 45077673A/G
5-3: 45089554T/G
5-3: 45179922T/G
5-3: 45257270C/T
5-3: 45366387G/A
5-3: 45367584C/G
5-3: 45381699C/A
5-3: 45407211G/A
5-3: 45518798T/C
5-3: 45519823G/A
5-3: 45625440G/A
5-3: 45636327T/A
5-3: 45659171G/C
5-3: 45724887C/T
5-3: 45760934C/A
5-3: 45849997A/G
5-3: 45873507A/C
5-3: 45884476A/T
5-3: 45895779G/A
5-3: 45931019C/A
5-3: 45937459T/C
5-3: 45971599G/T
5-3: 45990141G/A
5-3: 46005247C/A
5-3: 46020329A/T
5-3: 46153889A/G
5-3: 46156512T/C
5-3: 46183564G/A
5-3: 46252519C/T
5-3: 46291862A/G
5-3: 46366292G/A
5-3: 46394156T/A
5-3: 46431414A/G
5-3: 46457060C/T
5-3: 46506212T/C
5-3: 46536854A/T
5-3: 46565515C/A
5-3: 46596661T/G
5-3: 46624894G/T
5-3: 46691084C/G
5-3: 46738994C/T
5-3: 46754520C/T
5-3: 46832797A/T
5-3: 46867947C/T
5-3: 46901541G/C
5-3: 46917773G/A
5-3: 46949127T/G
5-3: 46978073C/T
5-3: 46992237T/C
5-3: 47048471G/C
5-3: 47089427A/G
5-3: 47127583A/G
5-3: 47293994G/C
5-3: 47302916T/A
5-3: 47402548G/A
5-3: 47489657G/A
5-3: 47517886A/G
5-3: 47518863A/C
5-3: 47528677T/A
5-3: 47532239A/C
5-3: 47574963C/A
5-3: 47645383T/C
5-3: 47706460A/C
5-3: 47833877G/A
5-3: 47839384C/G
5-3: 47839401T/A
5-3: 47895231G/A
5-3: 47934703T/A
5-3: 47956137C/T
5-3: 48100814C/T
5-3: 48234180A/G
5-3: 48316955T/C
5-3: 48341422G/T
5-3: 48403874G/A
5-3: 48431314C/T
5-3: 48459637G/C
5-3: 48478769G/A
5-3: 48512905G/A
5-3: 48515397C/T
5-3: 48585436A/G
5-3: 48617136G/A
5-3: 48708579T/G
5-3: 48735785T/C
5-3: 48787413T/C
5-3: 48926455A/G
5-3: 48948410T/G
5-3: 48967485T/C
5-3: 49031335C/T
5-3: 49098439A/C
5-3: 49127652C/T
5-3: 49141560C/T
5-3: 49207969T/C
5-3: 49233610C/G
5-3: 49278560C/A
5-3: 49314651C/T
5-3: 49340831T/C
5-3: 49385061C/G
5-3: 49449792T/C
5-3: 49491670T/C
5-3: 49540377T/C
5-3: 49577864G/A
5-3: 49643245T/C
5-3: 49659688A/G
5-3: 49682961G/A
5-3: 49713599G/A
5-3: 49715082C/T
5-3: 49740987T/A
5-3: 49784571C/T
5-3: 49849675T/C
5-3: 49865510A/C
5-3: 49968784G/A
5-3: 50026031G/T
5-3: 50120463C/T
5-3: 50155044T/G
5-3: 50156073C/A
5-3: 50237480A/G
5-3: 50256185C/T
5-3: 50340583C/T
5-3: 50347035T/C
5-3: 50369221G/A
5-3: 50449992A/G
5-3: 50481435A/T
5-3: 50552743A/G
5-3: 50592818G/A
5-3: 50620075C/G
5-3: 50658933T/A
5-3: 50671218G/A
5-3: 50698008T/C
5-3: 50710969A/T
5-3: 50724917G/A
5-3: 50753131A/G
5-3: 50790619C/T
5-3: 50834874T/A
5-3: 50835003G/A
5-3: 50856695G/C
5-3: 51048249G/T
5-3: 51050938A/G
5-3: 51065670A/G
5-3: 51080087G/C
5-3: 51102856G/A
5-3: 51126331T/C
5-3: 51153739T/C
5-3: 51188192T/A
5-3: 51243433T/C
5-3: 51299817T/C
5-3: 51317924A/T
5-3: 51390092A/T
5-3: 51454118A/G
5-3: 51487265C/G
5-3: 51529429C/T
5-3: 51615848T/A
5-3: 51650900C/T
5-3: 51681732A/T
5-3: 51726183A/G
5-3: 51770406A/G
5-3: 51772334G/A
5-3: 51773077C/T
5-3: 51774751T/C
5-3: 51787944T/G
5-3: 51827531A/G
5-3: 51862092C/T
5-3: 51875694G/T
5-3: 51977372C/T
5-3: 52079448C/A
5-3: 52155089T/G
5-3: 52313371G/C
5-3: 52449777G/A
5-3: 52531479C/T
5-3: 52649227T/A
5-3: 52654558C/T
5-3: 52655610C/A
5-3: 52656003A/G
5-3: 52656678G/A
5-3: 52656733G/A
5-3: 52659479C/T
5-3: 52738699A/T
5-3: 52796655A/C
5-3: 52854841C/A
5-3: 52873027C/G
5-3: 52889641G/T
5-3: 52893318T/C
5-3: 52925320T/C
5-3: 52927316T/C
5-3: 52954071G/A
5-3: 52968528C/T
5-3: 53026070C/T
5-3: 53038154A/G
5-3: 53051193A/G
5-3: 53051394G/A
5-3: 53051524T/G
5-3: 53125947A/T
5-3: 53164566A/T
5-3: 53167705G/A
5-3: 53174732A/T
5-3: 53224256T/A
5-3: 53270155G/T
5-3: 53340049G/A
5-3: 53395707A/G
5-3: 53479868T/C
5-3: 53488481T/A
5-3: 53516115C/A
5-3: 53533383T/C
5-3: 53542058G/A
5-3: 53542102C/T
5-3: 53545353G/A
5-3: 53556322A/T
5-3: 53559519C/T
5-3: 53569723T/C
5-3: 53589670A/G
5-3: 53652319A/C
5-3: 53714413C/T
5-3: 53734324C/T
5-3: 53748533A/G
5-3: 53775421G/T
5-3: 53799249C/T
5-3: 53805584T/A
5-3: 53843499C/T
5-3: 53855852T/C
5-3: 53869304A/T
5-3: 53905539A/G
5-3: 53912502C/G
5-3: 53912508G/A
5-3: 53938944T/A
5-3: 53975397T/C
5-3: 54017438T/A
5-3: 54064430G/A
5-3: 54082435C/T
5-3: 54096588A/G
5-3: 54207095G/A
5-3: 54247891C/G
5-3: 54301288A/G
5-3: 54333359G/A
5-3: 54360064G/A
5-3: 54456285A/G
5-3: 54483544A/G
5-3: 54518600T/G
5-3: 54529837G/A
5-3: 54540020C/T
5-3: 54565728T/A
5-3: 54608094G/A
5-3: 54648779A/G
5-3: 54660958T/C
5-3: 54859709T/C
5-3: 54868075G/A
5-3: 54871376T/C
5-3: 54880150A/G
5-3: 54888336A/T
5-3: 54903495A/G
5-3: 54906015C/T
5-3: 54908577C/T
5-3: 54984235C/T
5-3: 54999555G/A
5-3: 55010857A/T
5-3: 55025958T/C
5-3: 55058208C/A
5-3: 55104367A/G
5-3: 55107076G/C
5-3: 55149959T/C
5-3: 55184285T/A
5-3: 55252735A/T
5-3: 55279368G/A
5-3: 55359401C/T
5-3: 55401380A/G
5-3: 55523087C/G
5-3: 55569282G/A
5-3: 55594865A/G
5-3: 55605272A/G
5-3: 55636178T/C
5-3: 55775964G/A
5-3: 55897491A/C
5-3: 55914996A/G
5-3: 55934781T/A
5-3: 55966469C/G
5-3: 56042262T/C
5-3: 56056443C/T
5-3: 56142581G/T
5-3: 56167286A/T
5-3: 56196934A/G
5-3: 56363019T/A
5-3: 56425393A/C
5-3: 56486806A/G
5-3: 56505958G/A
5-3: 56533461C/A
5-3: 56682759A/T
5-3: 56718562G/C
5-3: 56812250C/T
5-3: 56839179C/A
5-3: 56889487G/T
5-3: 56940193T/C
5-3: 56977566T/C
5-3: 56977576A/T
5-3: 56996180G/A
5-3: 56999679A/G
5-3: 57015672A/C
5-3: 57021586C/T
5-3: 57025281T/G
5-3: 57028083A/G
5-3: 57039302T/C
5-3: 57095121G/C
5-3: 57109789A/G
5-3: 57171717G/A
5-3: 57287156C/A
5-3: 57291966C/T
5-3: 57318691T/C
5-3: 57376054C/A
5-3: 57377828C/T
5-3: 57402701C/T
5-3: 57418698T/C
5-3: 57456305T/A
5-3: 57496220T/C
5-3: 57525566G/A
5-3: 57567455C/G
5-3: 57582515A/T
5-3: 57595418G/A
5-3: 57596956T/C
5-3: 57598255T/C
5-3: 57612227G/A
5-3: 57646349C/T
5-3: 57672525G/A
5-3: 57728480C/T
5-3: 57846759G/A
5-3: 57853114C/T
5-3: 57857101T/G
5-3: 57878569G/T
5-3: 57893196A/G
5-3: 57922435C/T
5-3: 57935893T/C
5-3: 57979732T/A
5-3: 58024530T/A
5-3: 58089000A/C
5-3: 58158913G/A
5-3: 58188406A/G
5-3: 58233922A/C
5-3: 58269002A/C
5-3: 58303749C/T
5-3: 58389438T/A
5-3: 58505966C/G
5-3: 58517421G/A
5-3: 58539783G/T
5-3: 58579546T/C
5-3: 58631627C/A
5-3: 58700638T/C
5-3: 58731862T/G
5-3: 58768947G/A
5-3: 58793638G/A
5-3: 58818784T/A
5-3: 58861269T/C
5-3: 58957547A/T
5-3: 59012834A/G
5-3: 59154554C/T
5-3: 59325820T/C
5-3: 59351058A/C
5-3: 59365043G/C
5-3: 59444928T/A
5-3: 59505974T/C
5-3: 59554418G/T
5-3: 59561146T/A
5-3: 59591635A/C
5-3: 59609854T/A
5-3: 59629660A/T
5-3: 59643129T/A
5-3: 59653235C/A
5-3: 59726454G/A
5-3: 59760052G/A
5-3: 59779655G/A
5-3: 59798499A/G
5-3: 59820823A/T
5-3: 59833386G/A
5-3: 59866791G/A
5-3: 59930164A/G
5-3: 59992284A/G
5-3: 60046468A/G
5-3: 60053909G/A
5-3: 60114755G/C
5-3: 60253442C/T
5-3: 60274267C/G
5-3: 60344970C/T
5-3: 60364595T/C
5-3: 60397710C/A
5-3: 60403354G/A
5-3: 60416733G/C
5-3: 60439139T/A
5-3: 60459484A/G
5-3: 60477040T/C
5-3: 60600331C/A
5-3: 60628253T/G
5-3: 60650572T/C
5-3: 60700937T/A
5-3: 60752274A/T
5-3: 60791031G/T
5-3: 60839426C/G
5-3: 60897363T/C
5-3: 60910648A/T
5-3: 60958430A/G
5-3: 60976938C/T
5-3: 61008510G/C
5-3: 61044808T/G
5-3: 61078271C/T
5-3: 61096911T/C
5-3: 61110978C/A
5-3: 61146221C/T
5-3: 61180317C/T
5-3: 61214103A/T
5-3: 61259502C/A
5-3: 61275656G/A
5-3: 61325230A/G
5-3: 61383712A/G
5-3: 61467634G/A
5-3: 61550697C/T
5-3: 61625689T/A
5-3: 61659612G/A
5-3: 61767906T/G
5-3: 61835439A/G
5-3: 61881961G/A
5-3: 61972440G/A
5-3: 61995218G/A
5-3: 62041245A/G
5-3: 62068023A/T
5-3: 62120694C/T
5-3: 62162520T/A
5-3: 62214865A/G
5-3: 62312457C/A
5-3: 62331228A/G
5-3: 62505429A/T
5-3: 62555229C/T
5-3: 62566332A/G
5-3: 62783522C/T
5-3: 62794299A/G
5-3: 62842730T/C
5-3: 62855466C/T
5-3: 62886398T/A
5-3: 62909693A/G
5-3: 62909922G/T
5-3: 62910215A/C
5-3: 62910221A/G
5-3: 62910437T/A
5-3: 62946890A/G
5-3: 63019886A/G
5-3: 63077801C/A
5-3: 63122466A/T
5-3: 63159332A/G
5-3: 63222513G/T
5-3: 63263782C/T
5-3: 63437284T/C
5-3: 63556963T/C
5-3: 63572621A/G
5-3: 63604258C/A
5-3: 63633476A/C
5-3: 63673501G/A
5-3: 63700730A/G
5-3: 63749084A/G
5-3: 63800792T/A
5-3: 63840769T/C
5-3: 63857765G/A
5-3: 64061949C/T
5-3: 64116181G/C
5-3: 64223512T/G
5-3: 64251419T/C
5-3: 64264016C/T
5-3: 64317224A/G
5-3: 64390010G/T
5-3: 64437654T/A
5-3: 64593624C/A
5-3: 64653669A/G
5-3: 64667523T/C
5-3: 64689808T/G
5-3: 64827095G/C
5-3: 64869097T/C
5-3: 64920039C/T
5-3: 64960414T/C
5-3: 65090430C/T
5-3: 65153888C/T
5-3: 65187621T/C
5-3: 65234471G/A
5-3: 65235550C/T
5-3: 65235565C/T
5-3: 65296040A/G
5-3: 65309145T/G
5-3: 65357027C/A
5-3: 65516099C/A
5-3: 65572846A/C
5-3: 65573794T/C
5-3: 65574596T/C
5-3: 65574697T/C
5-3: 65584158T/C
5-3: 65614288G/A
5-3: 65633507C/T
5-3: 65647067A/G
5-3: 65698000C/T
5-3: 65749605A/T
5-3: 65784949C/A
5-3: 65858052C/T
5-3: 65943336A/T
5-3: 65957325C/G
5-3: 66012591C/T
5-3: 66062439T/C
5-3: 66112451G/A
5-3: 66147455A/G
5-3: 66214946A/T
5-3: 66226530G/T
5-3: 66268410A/T
5-3: 66279625G/C
5-3: 66319686T/A
5-3: 66346203G/A
5-3: 66399222A/G
5-3: 66451302G/T
5-3: 66451316A/T
5-3: 66451321C/T
5-3: 66451338T/G
5-3: 66451344A/T
5-3: 66451346G/T
5-3: 66451367C/T
5-3: 66451401C/T
5-3: 66451403A/C
5-3: 66637322C/T
5-3: 66669806A/T
5-3: 66728533T/C
5-3: 66800436C/T
5-3: 66839303C/A
5-3: 66869028A/G
5-3: 66915674T/C
5-3: 67032461T/C
5-3: 67166544A/G
5-3: 67504253G/A
5-3: 67615537C/T
5-3: 67742548G/A
5-3: 67767098T/C
5-3: 67869892C/T
5-3: 67874186G/A
5-3: 68052511C/G
5-3: 68138766T/C
5-3: 68284334T/C
5-3: 68331955G/T
5-3: 68396257T/A
5-3: 68421974C/T
5-3: 68533704T/C
5-3: 68549983G/A
5-3: 68558288A/G
5-3: 68568950C/T
5-3: 68619025T/C
5-3: 68662827A/C
5-3: 68736580A/C
5-3: 68768683A/T
5-3: 68918674C/A
5-3: 69023071G/T
5-3: 69054679C/T
5-3: 69055228T/C
5-3: 69075519C/T
5-3: 69089540C/T
5-3: 69101177A/G
5-3: 69116764G/A
5-3: 69132443A/T
5-3: 69201739T/C
5-3: 69342187G/T
5-3: 69447854T/G
5-3: 69808362C/T
5-3: 69901926T/C
5-4: 400764G/A
5-4: 695221A/G
5-4: 803265T/C
5-4: 838686G/A
5-4: 1046815G/A
5-4: 1078249A/C
5-4: 1096932C/T
5-4: 1295562C/A
5-4: 1306107C/A
5-4: 1516671T/C
5-4: 1635699C/T
5-4: 1695201A/T
5-4: 1706685C/T
5-4: 2163978G/C
5-4: 2238753T/C
5-4: 2279620A/G
5-4: 2291102C/A
5-4: 2295272T/A
5-4: 2296511T/C
5-4: 2303555A/G
5-4: 2318158A/C
5-4: 2324021A/G
5-4: 2370624T/A
5-4: 2599749G/A
5-4: 2728991A/G
5-4: 2729708T/A
5-4: 2741454G/C
5-4: 2757747G/A
5-4: 2782799C/T
5-4: 2872882G/A
5-4: 2961335A/G
5-4: 3039149A/G
5-4: 3273460T/C
5-4: 3463667A/C
5-4: 3680981A/G
5-4: 3701897T/A
5-4: 3719939T/C
5-4: 3800661G/A
5-4: 3853983C/T
5-4: 3897753C/T
5-4: 3962359A/G
5-4: 4046816C/T
5-4: 4137342T/G
5-4: 4138112G/.
5-4: 4153532A/G
5-4: 4175483C/A
5-4: 4259086T/C
5-4: 4485758G/T
5-4: 4528166A/G
5-4: 4622529G/A
5-4: 4663543A/G
5-4: 4736226A/G
5-4: 4803012T/C
5-4: 4833938C/T
5-4: 4840587T/G
5-4: 4916083T/G
5-4: 4963994C/T
5-4: 5062378T/G
5-4: 5077550C/T
5-4: 5213370C/T
5-4: 5357948T/A
5-4: 5436165C/T
5-4: 5439980T/C
5-4: 5483672A/G
5-4: 5772241G/A
5-4: 5876052G/T
5-4: 5907544C/T
5-4: 5938095C/T
5-4: 6036410T/C
5-4: 6131917A/G
5-4: 6171321A/C
5-4: 6206957A/C
5-4: 6263370G/A
5-4: 6292175C/T
5-4: 6316950A/T
5-4: 6332856C/T
5-4: 6350087A/G
5-4: 6350441G/A
5-4: 6350444G/A
5-4: 6351899G/A
5-4: 6390418G/A
5-4: 6440676T/A
5-4: 6469420T/C
5-4: 6492178G/A
5-4: 6522591C/T
5-4: 6541697G/C
5-4: 6691832T/G
5-4: 6745857C/T
5-4: 6885812G/T
5-4: 7050043C/T
5-4: 7121220G/A
5-4: 7136636T/G
5-4: 7154538A/G
5-4: 7223224C/G
5-4: 7249102A/G
5-4: 7278476A/T
5-4: 7278621T/C
5-4: 7300473T/C
5-4: 7334629C/A
5-4: 7364529G/A
5-4: 7399129G/T
5-4: 7463988C/T
5-4: 7589106G/A
5-4: 7599144G/A
5-4: 7654922C/T
5-4: 7719960C/T
5-4: 7722773G/A
5-4: 7725326C/T
5-4: 7726752C/T
5-4: 7727475T/A
5-4: 7729342G/A
5-4: 7730719C/T
5-4: 7731669G/A
5-4: 7732255C/G
5-4: 7732773G/A
5-4: 7733556C/A
5-4: 7765564A/C
5-4: 7804626A/G
5-4: 7854819A/G
5-4: 7889919C/T
5-4: 7920684G/A
5-4: 8065923T/C
5-4: 8070337A/G
5-4: 8081104A/G
5-4: 8172646A/T
5-4: 8181940A/T
5-4: 8483326A/G
5-4: 8582357T/C
5-4: 8592814T/G
5-4: 8641423C/T
5-4: 8649232A/G
5-4: 8652987G/A
5-4: 8780733C/T
5-4: 8787305C/G
5-4: 8847193A/T
5-4: 8872524T/C
5-4: 8909552T/A
5-4: 8970675C/A
5-4: 9016501T/C
5-4: 9033908C/T
5-4: 9050400C/T
5-4: 9067296A/T
5-4: 9203112C/T
5-4: 9262499T/C
5-4: 9330757C/T
5-4: 9374816C/T
5-4: 9499974A/G
5-4: 9584494G/T
5-4: 9727346C/G
5-4: 9939565A/C
5-4: 9998790T/A
5-4: 10113184T/C
5-4: 10125708C/A
5-4: 10182234G/A
5-4: 10438647A/G
5-4: 10475938G/T
5-4: 10521546A/G
5-4: 10569726C/G
5-4: 10604092T/A
5-4: 10666550C/T
5-4: 10711195T/C
5-4: 10754107T/G
5-4: 10813646T/C
5-4: 10870232C/T
5-4: 10914981G/T
5-4: 10941089C/T
5-4: 10964694T/C
5-4: 10985128T/C
5-4: 11027961G/A
5-4: 11057653T/C
5-4: 11077835A/C
5-4: 11142434G/A
5-4: 11292659G/A
5-4: 11375482A/G
5-4: 11443438T/C
5-4: 11606196G/T
5-4: 11632513A/G
5-4: 11726012T/C
5-4: 11751680G/T
5-4: 11787428C/T
5-4: 11883724T/C
5-4: 11960289T/C
5-4: 12015439C/T
5-4: 12182467C/A
5-4: 12234504G/A
5-4: 12240602C/A
5-4: 12242982A/G
5-4: 12259383A/C
5-4: 12336442G/A
5-4: 12385120C/T
5-4: 12436593T/C
5-4: 12503761G/T
5-4: 12522835A/T
5-4: 12578041C/T
5-4: 12616069C/T
5-4: 12621170G/A
5-4: 12627056G/A
5-4: 12658773T/C
5-4: 12837402T/A
5-4: 12935448A/T
5-4: 12979848C/A
5-4: 12979856T/G
5-4: 13039228T/C
5-4: 13187741C/G
5-4: 13216982G/T
5-4: 13281213A/G
5-4: 13307759T/C
5-4: 13348556G/A
5-4: 13444239A/G
5-4: 13454853A/G
5-4: 13471391C/T
5-4: 13487262C/T
5-4: 13498171G/T
5-4: 13587317A/G
5-4: 13668056A/T
5-4: 13705644C/T
5-4: 13740441T/G
5-4: 13805453G/A
5-4: 13832978C/T
5-4: 13872050A/G
5-4: 13948090A/C
5-4: 13993392A/T
5-4: 14009186G/T
5-4: 14030881A/G
5-4: 14070373T/C
5-4: 14147511C/T
5-4: 14186416C/G
5-4: 14280399A/G
5-4: 14287673G/A
5-4: 14318516G/T
5-4: 14449570A/G
5-4: 14473181A/G
5-4: 14487619C/T
5-4: 14518887T/C
5-4: 14563880C/A
5-4: 14619914G/C
5-4: 14686219G/A
5-4: 14742050T/A
5-4: 14751615G/A
5-4: 14782826A/C
5-4: 14800852G/A
5-4: 14800876T/C
5-4: 14813850A/T
5-4: 14814406A/T
5-4: 14824110G/C
5-4: 14834543T/G
5-4: 14947796T/C
5-4: 14951202C/T
5-4: 14965124G/A
5-4: 15068248T/A
5-4: 15166747G/T
5-4: 15218769A/C
5-4: 15337252C/G
5-4: 15420596C/T
5-4: 15478681G/T
5-4: 15488888A/G
5-4: 15505871G/A
5-4: 15531618A/T
5-4: 15553167A/G
5-4: 15580427G/A
5-4: 15593413C/G
5-4: 15649633A/T
5-4: 15653864A/T
5-4: 15673236G/A
5-4: 15688872C/G
5-4: 15699161C/T
5-4: 15735207C/A
5-4: 15810680G/A
5-4: 15845537C/T
5-4: 15859417A/G
5-4: 15892125G/A
5-4: 15920543A/G
5-4: 15920553G/A
5-4: 15951596T/A
5-4: 16030239T/C
5-4: 16093505G/A
5-4: 16094983A/G
5-4: 16095391G/A
5-4: 16100173T/A
5-4: 16100211T/C
5-4: 16100225A/G
5-4: 16100231G/A
5-4: 16100232C/T
5-4: 16100301A/G
5-4: 16110055A/C
5-4: 16147722G/T
5-4: 16177503A/G
5-4: 16212017G/T
5-4: 16266461C/T
5-4: 16304674T/C
5-4: 16332010C/T
5-4: 16338641C/T
5-4: 16350238A/G
5-4: 16360771T/G
5-4: 16386834T/G
5-4: 16397096T/C
5-4: 16429111G/A
5-4: 16461982C/T
5-4: 16462743T/C
5-4: 16479447G/A
5-4: 16509007A/G
5-4: 16509474A/C
5-4: 16522656C/T
5-4: 16548003G/A
5-4: 16556084C/T
5-4: 16611517T/G
5-4: 16617123A/T
5-4: 16624884A/C
5-4: 16624960T/C
5-4: 16624967G/T
5-4: 16625103A/G
5-4: 16625153C/T
5-4: 16625234T/C
5-4: 16625330G/A
5-4: 16625348T/C
5-4: 16625372C/G
5-4: 16625391G/A
5-4: 16625463T/G
5-4: 16625494C/A
5-4: 16625525A/C
5-4: 16625531G/A
5-4: 16625666C/G
5-4: 16625700A/G
5-4: 16625705G/A
5-4: 16625717A/G
5-4: 16625800A/T
5-4: 16625968G/A
5-4: 16625993A/T
5-4: 16627216T/C
5-4: 16649172A/G
5-4: 16661536G/A
5-4: 16673980G/C
5-4: 16691412A/T
5-4: 16710755T/C
5-4: 16759581C/G
5-4: 16794713G/C
5-4: 16804485C/G
5-4: 16810529A/G
5-4: 16813098A/T
5-4: 16817222C/T
5-4: 16826724A/T
5-4: 16826793T/A
5-4: 16839353C/T
5-4: 16853713T/C
5-4: 16911136T/A
5-4: 16936187T/C
5-4: 16947698A/C
5-4: 16967634A/T
5-4: 16980490T/A
5-4: 16991067T/A
5-4: 16993000T/C
5-4: 17069963G/T
5-4: 17243448G/T
5-4: 17246790A/G
5-4: 17257211T/C
5-4: 17291198C/T
5-4: 17377515C/A
5-4: 17474530T/A
5-4: 17485229C/T
5-4: 17503838G/A
5-4: 17525803G/A
5-4: 17679704C/T
5-4: 17703154A/G
5-4: 17832271C/T
5-4: 17872546A/C
5-4: 17885726G/T
5-4: 17902014C/T
5-4: 17934700G/T
5-4: 17957051T/C
5-4: 18012566C/A
5-4: 18033898G/A
5-4: 18087829A/T
5-4: 18148615T/G
5-4: 18179708T/C
5-4: 18208205G/A
5-4: 18259012T/G
5-4: 18320327C/G
5-4: 18376123C/T
5-4: 18437143A/T
5-4: 18464275T/G
5-4: 18517887G/A
5-4: 18566076A/T
5-4: 18605516T/A
5-4: 18671592T/G
5-4: 18671640A/T
5-4: 18671651T/C
5-4: 18671840T/C
5-4: 18671888T/C
5-4: 18671984A/G
5-4: 18693103G/A
5-4: 18722879A/G
5-4: 18845702T/C
5-4: 18952907C/T
5-4: 18955072G/T
5-4: 18991095T/A
5-4: 19014933T/C
5-4: 19038112A/G
5-4: 19054263A/T
5-4: 19066759C/T
5-4: 19091413C/T
5-4: 19160219C/T
5-4: 19209995C/T
5-4: 19406032C/G
5-4: 19493661G/A
5-4: 19504519A/T
5-4: 19533141C/A
5-4: 19659266A/G
5-4: 19696074T/C
5-4: 19751111G/A
5-4: 19786737A/G
5-4: 19816094T/A
5-4: 19848500A/T
5-4: 19888953A/T
5-4: 19901793T/C
5-4: 19994595A/G
5-4: 20005113G/C
5-4: 20134852G/T
5-4: 20143441G/A
5-4: 20199153T/C
5-4: 20242578C/A
5-4: 20365580T/C
5-4: 20430091G/C
5-4: 20527154A/T
5-4: 20558170G/T
5-4: 20584024A/G
5-4: 20668097A/T
5-4: 20744187A/T
5-4: 20816482G/A
5-4: 20829158C/T
5-4: 20874666C/T
5-4: 20969973G/T
5-4: 20977658C/T
5-4: 20991346T/C
5-4: 21005825A/G
5-4: 21036568T/C
5-4: 21110757A/T
5-4: 21125823A/C
5-4: 21137918A/G
5-4: 21139957C/T
5-4: 21205786T/A
5-4: 21252154C/T
5-4: 21268396G/T
5-4: 21333883T/G
5-4: 21380896C/T
5-4: 21416756A/G
5-4: 21445112T/G
5-4: 21474547G/A
5-4: 21485609A/C
5-4: 21520689T/G
5-4: 21550074A/G
5-4: 21621068G/A
5-4: 21642236C/T
5-4: 21654083A/T
5-4: 21664642G/A
5-4: 21673311C/T
5-4: 21673612C/T
5-4: 21676782T/A
5-4: 21689457T/G
5-4: 21708829A/C
5-4: 21722915A/G
5-4: 21726447C/T
5-4: 21733119A/C
5-4: 21733733C/A
5-4: 21746375A/G
5-4: 21763090T/C
5-4: 21764714G/A
5-4: 21774001C/T
5-4: 21781013A/C
5-4: 21791490C/T
5-4: 21804074A/T
5-4: 21847730G/C
5-4: 21867954C/T
5-4: 21880843T/C
5-4: 21894436G/A
5-4: 21949858G/T
5-4: 22004145A/T
5-4: 22069566T/A
5-4: 22085260T/G
5-4: 22106952A/G
5-4: 22118371G/A
5-4: 22130028C/T
5-4: 22143374C/T
5-4: 22177204G/A
5-4: 22191535C/T
5-4: 22206175C/A
5-4: 22216413C/G
5-4: 22233447G/A
5-4: 22247115G/A
5-4: 22269048A/G
5-4: 22294198G/T
5-4: 22322204C/T
5-4: 22332492G/C
5-4: 22345183G/A
5-4: 22368527C/T
5-4: 22377055G/T
5-4: 22383555C/T
5-4: 22400471A/G
5-4: 22419597C/A
5-4: 22434401C/A
5-4: 22445929A/G
5-4: 22475516A/T
5-4: 22487914C/T
5-4: 22496076T/C
5-4: 22498447T/A
5-4: 22524899C/T
5-4: 22531812C/G
5-4: 22536474G/A
5-4: 22548991C/T
5-4: 22563737G/C
5-4: 22570075C/T
5-4: 22574269A/G
5-4: 22602954G/T
5-4: 22622882C/T
5-4: 22644232A/G
5-4: 22673817C/T
5-4: 22724684G/A
5-4: 22742162A/G
5-4: 22759716G/C
5-4: 22771477C/T
5-4: 22772739C/T
5-4: 22781843G/A
5-4: 22793048A/T
5-4: 22808946T/A
5-4: 22808983G/C
5-4: 22831454T/A
5-4: 22898861A/G
5-4: 23048203A/T
5-4: 23091894T/G
5-4: 23091927C/T
5-4: 23092307T/C
5-4: 23092405C/T
5-4: 23092615A/G
5-4: 23092836G/T
5-4: 23092849A/G
5-4: 23092966G/A
5-4: 23112330T/G
5-4: 23136041T/G
5-4: 23152606A/C
5-4: 23152655A/T
5-4: 23152657C/T
5-4: 23152672T/G
5-4: 23152674C/A
5-4: 23152695C/A
5-4: 23152703T/C
5-4: 23152733C/.
5-4: 23152747A/.
5-4: 23152809A/T
5-4: 23152812A/T
5-4: 23152824T/A
5-4: 23152825G/A
5-4: 23152828T/C
5-4: 23152829G/A
5-4: 23152845G/.
5-4: 23152885T/C
5-4: 23152888G/A
5-4: 23153246T/C
5-4: 23153314T/G
5-4: 23153435C/.
5-4: 23153438T/.
5-4: 23153446G/.
5-4: 23153869G/T
5-4: 23153944T/C
5-4: 23153969A/.
5-4: 23153975T/.
5-4: 23153987T/.
5-4: 23154007C/.
5-4: 23154010T/.
5-4: 23154011T/.
5-4: 23154105T/C
5-4: 23154174T/A
5-4: 23154231G/C
5-4: 23154320G/A
5-4: 23154479C/A
5-4: 23154545A/C
5-4: 23154556G/A
5-4: 23154571G/C
5-4: 23154578G/A
5-4: 23169624A/T
5-4: 23191970G/A
5-4: 23202417T/G
5-4: 23210955A/G
5-4: 23213408A/C
5-4: 23223843C/T
5-4: 23224334A/G
5-4: 23245978G/A
5-4: 23256567T/A
5-4: 23274345A/T
5-4: 23296714T/A
5-4: 23316762T/C
5-4: 23327876C/T
5-4: 23345323C/A
5-4: 23345332A/T
5-4: 23441223G/A
5-4: 23447307G/A
5-4: 23451449G/A
5-4: 23453436C/T
5-4: 23463849A/T
5-4: 23474168C/T
5-4: 23513731A/C
5-4: 23524734C/T
5-4: 23547484A/G
5-4: 23565882C/A
5-4: 23622400G/C
5-4: 23643850C/T
5-4: 23672486G/T
5-4: 23717861C/A
5-4: 23742205T/C
5-4: 23762508C/T
5-4: 23793701A/G
5-4: 23835966C/T
5-4: 23861537T/C
5-4: 23882176G/A
5-4: 23941954A/G
5-4: 23957472C/T
5-4: 23990138G/T
5-4: 24076802T/G
5-4: 24110880A/G
5-4: 24138493T/A
5-4: 24165636T/C
5-4: 24226658C/T
5-4: 24291528G/C
5-4: 24318372G/A
5-4: 24357769C/G
5-4: 24359639C/A
5-4: 24367553C/T
5-4: 24381253A/C
5-4: 24421743A/C
5-4: 24449231T/A
5-4: 24526499C/T
5-4: 24543974G/A
5-4: 24554790C/T
5-4: 24555165G/A
5-4: 24555174G/A
5-4: 24565174T/G
5-4: 24576945G/T
5-4: 24595190A/G
5-4: 24650105C/T
5-4: 24662940T/C
5-4: 24699694G/C
5-4: 24768843T/C
5-4: 24780167T/C
5-4: 24795946T/C
5-4: 24810020C/T
5-4: 24814483A/G
5-4: 24825625T/G
5-4: 24838041G/A
5-4: 24849277A/T
5-4: 24885270A/T
5-4: 24929070G/A
5-4: 24944199G/C
5-4: 24956141C/T
5-4: 24994481T/C
5-4: 25063369C/T
5-4: 25094999C/A
5-4: 25095066A/G
5-4: 25095068T/C
5-4: 25095128T/C
5-4: 25095147T/C
5-4: 25095152T/C
5-4: 25095168A/G
5-4: 25095171G/A
5-4: 25095177C/A
5-4: 25095203T/C
5-4: 25095240G/T
5-4: 25095477T/.
5-4: 25095483G/A
5-4: 25095541G/.
5-4: 25095563C/A
5-4: 25095588C/A
5-4: 25095590T/G
5-4: 25095601T/C
5-4: 25095604C/T
5-4: 25095609G/A
5-4: 25095617G/A
5-4: 25095618A/G
5-4: 25095620C/T
5-4: 25095623G/A
5-4: 25095661C/T
5-4: 25095675A/G
5-4: 25095694G/A
5-4: 25095703C/A
5-4: 25095710A/G
5-4: 25095734C/A
5-4: 25095738A/T
5-4: 25095741G/T
5-4: 25095761T/C
5-4: 25110006T/C
5-4: 25113158A/T
5-4: 25113179T/G
5-4: 25113207C/A
5-4: 25113212C/T
5-4: 25113261T/C
5-4: 25113293G/A
5-4: 25113299T/A
5-4: 25113465G/A
5-4: 25113547A/.
5-4: 25113557A/T
5-4: 25113596A/T
5-4: 25123510G/T
5-4: 25124621G/T
5-4: 25135786A/G
5-4: 25143982G/A
5-4: 25148301A/G
5-4: 25167785C/G
5-4: 25202105G/A
5-4: 25215114A/C
5-4: 25228837G/A
5-4: 25252824G/T
5-4: 25279857A/T
5-4: 25291401C/T
5-4: 25315938G/A
5-4: 25320934C/T
5-4: 25326945T/C
5-4: 25342394A/T
5-4: 25354337C/T
5-4: 25368281A/G
5-4: 25384695A/G
5-4: 25399336A/T
5-4: 25401028T/C
5-4: 25432915C/G
5-4: 25463087T/A
5-4: 25466746C/A
5-4: 25466752A/C
5-4: 25482981T/C
5-4: 25501769C/A
5-4: 25515086A/T
5-4: 25543185T/A
5-4: 25562448A/C
5-4: 25563553C/T
5-4: 25575352A/G
5-4: 25621801A/T
5-4: 25639627C/T
5-4: 25685797T/C
5-4: 25697047T/G
5-4: 25702658A/T
5-4: 25702659C/A
5-4: 25723765A/G
5-4: 25737747A/T
5-4: 25738665G/T
5-4: 25779116G/A
5-4: 25810077C/A
5-4: 25831109A/G
5-4: 25880306T/C
5-4: 25963915T/C
5-4: 25966232G/A
5-4: 26009274G/T
5-4: 26024700C/T
5-4: 26036259C/T
5-4: 26082401T/C
5-4: 26103888C/A
5-4: 26127672T/C
5-4: 26196934G/A
5-4: 26214340G/A
5-4: 26269755C/A
5-4: 26311729A/T
5-4: 26337335A/G
5-4: 26337339G/T
5-4: 26355255C/T
5-4: 26367697C/T
5-4: 26475974A/T
5-4: 26540945G/A
5-4: 26553129G/A
5-4: 26563690C/T
5-4: 26672926C/T
5-4: 26686934G/A
5-4: 26704519C/T
5-4: 26725789C/T
5-4: 26818616A/G
5-4: 27060523A/G
5-4: 27063497C/T
5-4: 27077159G/C
5-4: 27077720C/T
5-4: 27078321G/A
5-4: 27204416G/T
5-4: 27282813T/C
5-4: 27283315A/C
5-4: 27303634A/G
5-4: 27334371T/C
5-4: 27346245T/C
5-4: 27358373T/C
5-4: 27391253G/T
5-4: 27395821A/T
5-4: 27401416G/C
5-4: 27426431T/A
5-4: 27452893A/C
5-4: 27464775A/G
5-4: 27570446C/T
5-4: 27633240A/G
5-4: 27671149T/G
5-4: 27689884A/G
5-4: 27719875C/A
5-4: 27732717C/T
5-4: 27762610T/C
5-4: 27807273T/C
5-4: 27834914T/A
5-4: 28131000G/A
5-4: 28159598C/T
5-4: 28174275A/G
5-4: 28196884C/T
5-4: 28233379A/T
5-4: 28233784T/C
5-4: 28235234A/G
5-4: 28264317T/A
5-4: 28299045T/C
5-4: 28338116A/G
5-4: 28357403C/G
5-4: 28380020G/A
5-4: 28392364T/C
5-4: 28405129G/A
5-4: 28405846T/G
5-4: 28419380T/C
5-4: 28478558T/A
5-4: 28479164G/A
5-4: 28479185T/C
5-4: 28479200A/C
5-4: 28479203G/A
5-4: 28479251T/.
5-4: 28479280A/G
5-4: 28479333A/G
5-4: 28479368C/A
5-4: 28479444T/C
5-4: 28479655G/A
5-4: 28479684T/A
5-4: 28480383G/A
5-4: 28480398T/G
5-4: 28480445T/C
5-4: 28480451C/G
5-4: 28480486C/A
5-4: 28480516G/A
5-4: 28480570T/C
5-4: 28480594T/C
5-4: 28480617C/T
5-4: 28480618A/G
5-4: 28480623A/T
5-4: 28480673T/C
5-4: 28480705T/C
5-4: 28480715G/C
5-4: 28483383C/T
5-4: 28483465A/G
5-4: 28483468T/C
5-4: 28484262C/T
5-4: 28484416G/A
5-4: 28484426G/A
5-4: 28484493A/G
5-4: 28484524G/T
5-4: 28484608G/A
5-4: 28484625C/A
5-4: 28484634C/T
5-4: 28484700A/G
5-4: 28484702A/G
5-4: 28484759A/G
5-4: 28484761G/A
5-4: 28484773C/T
5-4: 28484803T/A
5-4: 28484833T/C
5-4: 28484865C/T
5-4: 28484888T/.
5-4: 28485009A/C
5-4: 28485165C/T
5-4: 28485177A/T
5-4: 28485206G/A
5-4: 28485209C/T
5-4: 28485239C/A
5-4: 28485290A/G
5-4: 28485293A/G
5-4: 28485294G/C
5-4: 28485352G/A
5-4: 28485357C/A
5-4: 28485359G/A
5-4: 28485363C/A
5-4: 28492453C/T
5-4: 28553976C/T
5-4: 28555903C/T
5-4: 28624927G/T
5-4: 28656938A/T
5-4: 28693611C/T
5-4: 28707808C/T
5-4: 28735597G/C
5-4: 28765753A/G
5-4: 28795662G/A
5-4: 28819866G/A
5-4: 28839850A/G
5-4: 28877187G/C
5-4: 29083837G/A
5-4: 29133371C/G
5-4: 29232184A/T
5-4: 29340273G/A
5-4: 29377444T/C
5-4: 29388965T/A
5-4: 29394799A/G
5-4: 29412271T/C
5-4: 29443107G/A
5-4: 29472578G/A
5-4: 29474659G/A
5-4: 29513613T/C
5-4: 29578886T/C
5-4: 29627213G/T
5-4: 29632488T/G
5-4: 29664884T/A
5-4: 29716887C/G
5-4: 29784462G/A
5-4: 29831691A/G
5-4: 29842555C/G
5-4: 29853798T/G
5-4: 29920611G/A
5-4: 29930564T/G
5-4: 29957342C/T
5-4: 29969187A/G
5-4: 30033189T/C
5-4: 30046122A/G
5-4: 30046680A/G
5-4: 30052002G/A
5-4: 30053882A/G
5-4: 30053937A/G
5-4: 30056279G/C
5-4: 30061923G/A
5-4: 30071758T/C
5-4: 30090244T/C
5-4: 30097759T/A
5-4: 30109551G/A
5-4: 30113708A/G
5-4: 30117873T/C
5-4: 30133088A/C
5-4: 30140895T/G
5-4: 30144303A/T
5-4: 30144306G/C
5-4: 30144311A/T
5-4: 30144315T/.
5-4: 30144346G/A
5-4: 30144444C/T
5-4: 30144481A/T
5-4: 30144489C/T
5-4: 30144621G/T
5-4: 30144989C/G
5-4: 30145035A/G
5-4: 30145081G/A
5-4: 30181973A/G
5-4: 30233093G/A
5-4: 30269593G/T
5-4: 30291599A/G
5-4: 30407659A/G
5-4: 30460434T/A
5-4: 30501915C/T
5-4: 30681322G/A
5-4: 30681474A/T
5-4: 30867111C/A
5-4: 31409549C/A
5-4: 31488309A/G
5-4: 31545008C/T
5-4: 31574932C/T
5-4: 31662963A/T
5-4: 31678327C/T
5-4: 31700354C/T
5-4: 31713088T/C
5-4: 31726193C/G
5-4: 31739596T/A
5-4: 31769774C/T
5-4: 31784009C/T
5-4: 31791751C/T
5-4: 31793061G/A
5-4: 31799036C/T
5-4: 31807080G/C
5-4: 31807432G/A
5-4: 31809748G/C
5-4: 31813157G/T
5-4: 31831390A/G
5-4: 31854439C/G
5-4: 31858971A/T
5-4: 31864810G/T
5-4: 31886591C/T
5-4: 31908461A/T
5-4: 31936459T/C
5-4: 31985405A/G
5-4: 32048090G/T
5-4: 32115874G/A
5-4: 32133316A/G
5-4: 32144523C/A
5-4: 32190251A/G
5-4: 32253160A/G
5-4: 32270539T/A
5-4: 32297643A/C
5-4: 32323808A/T
5-4: 32351131A/G
5-4: 32363698T/A
5-4: 32460290C/G
5-4: 32519053T/C
5-4: 32548388C/T
5-4: 32559825G/T
5-4: 32603849A/G
5-4: 32739912T/A
5-4: 32778209G/A
5-4: 32812574A/G
5-4: 32871632C/A
5-4: 32886124C/T
5-4: 32922401T/C
5-4: 32951194C/G
5-4: 33142569C/T
5-4: 33264250C/T
5-4: 33280662A/T
5-4: 33295196T/A
5-4: 33323836C/T
5-4: 33353640A/G
5-4: 33366038C/T
5-4: 33387420G/C
5-4: 33486161C/T
5-4: 33631047G/A
5-4: 33670570G/A
5-4: 33777381C/T
5-4: 33826800C/A
5-4: 33832686C/T
5-4: 33887262A/G
5-4: 34235421C/T
5-4: 34236740C/A
5-4: 34455014G/A
5-4: 34536322T/A
5-4: 34623689T/C
5-4: 34654312T/C
5-4: 34716129A/T
5-4: 34729474T/C
5-4: 34805630A/T
5-4: 34931787T/C
5-4: 34946899A/G
5-4: 34961354T/C
5-4: 34973943T/C
5-4: 34994861G/A
5-4: 35021909T/C
5-4: 35034457G/A
5-4: 35177501G/T
5-4: 35232648C/T
5-4: 35258031A/G
5-4: 35285682G/A
5-4: 35299018A/G
5-4: 35335244G/A
5-4: 35340846T/G
5-4: 35340902T/A
5-4: 35358517A/G
5-4: 35392766G/A
5-4: 35404308G/A
5-4: 35436907A/G
5-4: 35464934A/T
5-4: 35518368A/G
5-4: 35558040C/A
5-4: 35583909A/G
5-4: 35650116C/T
5-4: 35668270G/A
5-4: 35678423G/T
5-4: 35748630C/A
5-4: 35841434T/C
5-4: 35849049G/A
5-4: 35862155G/A
5-4: 35901508G/A
5-4: 35933703G/A
5-4: 35984859T/C
5-4: 36037345T/A
5-4: 36102508G/A
5-4: 36115762C/T
5-4: 36138535A/G
5-4: 36238901C/A
5-4: 36418918G/A
5-4: 36489484A/T
5-4: 36504911G/A
5-4: 36520626G/A
5-4: 36522818G/A
5-4: 36630028G/A
5-4: 36631232C/T
5-4: 36646500C/T
5-4: 36680491C/A
5-4: 36707294G/T
5-4: 36718038A/G
5-4: 36738193A/C
5-4: 36785919C/T
5-4: 36831088C/A
5-4: 36862449C/T
5-4: 36911930G/A
5-4: 36958682G/A
5-4: 36995054T/A
5-4: 37011834C/T
5-4: 37036829T/A
5-4: 37079858G/A
5-4: 37134221G/C
5-4: 37200630G/A
5-4: 37221463G/C
5-4: 37232089G/A
5-4: 37247964G/A
5-4: 37261028G/T
5-4: 37267652T/C
5-4: 37288404T/A
5-4: 37335612A/G
5-4: 37404985A/G
5-4: 37405178C/T
5-4: 37419612A/G
5-4: 37503715G/C
5-4: 37519290T/C
5-4: 37553677T/C
5-4: 37573624A/T
5-4: 37602167C/T
5-4: 37616688A/G
5-4: 37619697G/T
5-4: 37682194A/T
5-4: 37697471T/G
5-4: 37764931G/A
5-4: 37922587A/G
5-4: 38084274T/C
5-4: 38167807C/T
5-4: 38239738G/T
5-4: 38332913G/T
5-4: 38405594C/A
5-4: 38465036C/T
5-4: 38493384T/.
5-4: 38540902A/T
5-4: 38568262A/T
5-4: 38653964G/A
5-4: 38712235C/T
5-4: 38724702G/A
5-4: 38744966A/C
5-4: 38757821T/C
5-4: 38921274A/G
5-4: 38954736T/G
5-4: 38965297G/A
5-4: 38987588A/G
5-4: 39043924A/T
5-4: 39048784A/T
5-4: 39061403G/A
5-4: 39117249T/G
5-4: 39160893C/T
5-4: 39171730A/T
5-4: 39213043G/T
5-4: 39242173T/G
5-4: 39284236A/C
5-4: 39327127T/C
5-4: 39376668G/C
5-4: 39459109A/G
5-4: 39477317A/G
5-4: 39477549G/A
5-4: 39487808T/A
5-4: 39499568G/A
5-4: 39598034A/T
5-4: 39757056G/A
5-4: 39775043T/C
5-4: 39794403A/T
5-4: 39811498A/C
5-4: 39853205T/C
5-4: 39874378A/G
5-4: 39887833T/C
5-4: 39898759T/C
5-4: 39970443G/A
5-4: 40000383A/G
5-4: 40102550T/A
5-4: 40118911A/G
5-4: 40155922T/C
5-4: 40206224C/T
5-4: 40254883A/G
5-4: 40281379C/A
5-4: 40382130T/G
5-4: 40385622G/A
5-4: 40386291A/T
5-4: 40386375A/G
5-4: 40386394G/A
5-4: 40386400A/T
5-4: 40386401T/C
5-4: 40386403T/C
5-4: 40386566A/G
5-4: 40386620T/C
5-4: 40386639A/G
5-4: 40398481A/C
5-4: 40455276T/C
5-4: 40485544A/G
5-4: 40520945C/G
5-4: 40580961T/A
5-4: 40581550T/C
5-4: 40614405A/G
5-4: 40656064C/T
5-4: 40682535G/T
5-4: 40684758T/G
5-4: 40725436G/A
5-4: 40739941T/C
5-4: 40769766C/G
5-4: 40829925C/T
5-4: 40841184G/C
5-4: 40855116A/G
5-4: 40902174T/A
5-4: 41003420C/T
5-4: 41059479G/C
5-4: 41069526T/C
5-4: 41106544G/A
5-4: 41136745G/A
5-4: 41185621G/T
5-4: 41203078A/G
5-4: 41240416G/C
5-4: 41280946C/A
5-4: 41283366A/T
5-4: 41311611G/A
5-4: 41340478C/T
5-4: 41459855C/T
5-4: 41530246A/G
5-4: 41646355C/G
5-4: 41683562C/T
5-4: 41752424G/C
5-4: 41824654C/T
5-4: 41825800A/G
5-4: 41903856G/C
5-4: 41960437G/A
5-4: 42014047C/T
5-4: 42237939G/T
5-4: 42250088A/G
5-4: 42265503A/C
5-4: 42323723G/A
5-4: 42382930G/T
5-4: 42393483G/A
5-4: 42433965C/G
5-4: 42452146G/A
5-4: 42471371A/T
5-4: 42522519G/C
5-4: 42609371C/T
5-4: 42614068G/A
5-4: 42662557G/C
5-4: 42708246C/A
5-4: 42735231C/A
5-4: 42764776T/C
5-4: 42777369C/T
5-4: 42778373A/G
5-4: 42781150A/C
5-4: 42788883T/C
5-4: 42803146C/T
5-4: 42822039C/T
5-4: 42867464C/A
5-4: 42915298C/G
5-4: 42927137C/T
5-4: 42927436G/A
5-4: 42938769A/T
5-4: 42973124C/T
5-4: 43016424G/A
5-4: 43044146T/A
5-4: 43076097G/A
5-4: 43153213G/C
5-4: 43224424G/A
5-4: 43234296G/A
5-4: 43240256C/T
5-4: 43255551C/T
5-4: 43335869T/C
5-4: 43365012G/A
5-4: 43377925C/T
5-4: 43475009T/A
5-4: 43512723T/G
5-4: 43553432A/G
5-4: 43561384G/A
5-4: 43587993G/A
5-4: 43623493A/G
5-4: 43638976A/G
5-4: 43712846G/A
5-4: 43738080A/C
5-4: 43750677A/G
5-4: 43768637G/A
5-4: 43770783C/G
5-4: 43772148T/C
5-4: 43824049T/G
5-4: 43844372G/A
5-4: 43871368T/C
5-4: 43899593T/C
5-4: 43926974G/A
5-4: 43941469A/C
5-4: 43992737C/A
5-4: 44014764T/G
5-4: 44026392A/T
5-4: 44026548T/A
5-4: 44071921T/C
5-4: 44135598A/C
5-4: 44158407A/T
5-4: 44158626G/T
5-4: 44176749C/T
5-4: 44208916A/G
5-4: 44331649A/G
5-4: 44369966G/T
5-4: 44405491A/T
5-4: 44431342A/G
5-4: 44492683A/T
5-4: 44601978T/G
5-4: 44605213A/T
5-4: 44624416A/G
5-4: 44649107A/G
5-4: 44711869C/T
5-4: 44751031T/C
5-4: 44835797T/A
5-4: 44856937A/T
5-4: 44887199T/C
5-4: 44922771A/G
5-4: 44933604A/G
5-4: 44992640A/G
5-4: 45040263T/C
5-4: 45139678G/A
5-4: 45140540A/C
5-4: 45284440G/A
5-4: 45306841T/C
5-4: 45331085T/A
5-4: 45367584T/C
5-4: 45457980A/G
5-4: 45546933T/G
5-4: 45594425C/T
5-4: 45621157T/G
5-4: 45662253T/G
5-4: 45703147T/A
5-4: 45742617C/T
5-4: 45798400G/A
5-4: 45840603T/C
5-4: 45913679A/T
5-4: 45977454T/C
5-4: 45988601A/G
5-4: 46026204T/A
5-4: 46052618C/T
5-4: 46082230G/A
5-4: 46188063G/A
5-4: 46272380T/C
5-4: 46284318G/A
5-4: 46346992C/T
5-4: 46354129A/C
5-4: 46365129C/T
5-4: 46375674C/G
5-4: 46404766G/A
5-4: 46434813G/A
5-4: 46466461G/A
5-4: 46476540G/A
5-4: 46504386C/T
5-4: 46622332T/A
5-4: 46636544G/T
5-4: 46752758T/A
5-4: 46789214T/A
5-4: 46833054G/A
5-4: 46939219C/T
5-4: 46959465T/C
5-4: 47106225A/G
5-4: 47161831A/G
5-4: 47231968G/A
5-4: 47256372T/G
5-4: 47396022T/C
5-4: 47401402C/T
5-4: 47466876T/A
5-4: 47503027A/G
5-4: 47525546C/T
5-4: 47525549A/T
5-4: 47540245T/C
5-4: 47556009G/T
5-4: 47583698G/C
5-4: 47620989C/T
5-4: 47670409A/T
5-4: 47710529A/G
5-4: 47762583T/A
5-4: 47813607T/C
5-4: 47849619T/C
5-4: 47877479T/G
5-4: 47912468T/A
5-4: 47946089G/A
5-4: 47987559C/T
5-4: 48018405T/G
5-4: 48086511T/C
5-4: 48125007C/A
5-4: 48138521T/C
5-4: 48154246C/T
5-4: 48166949A/G
5-4: 48203657A/C
5-4: 48256716G/A
5-4: 48276882A/G
5-4: 48293859C/A
5-4: 48327144T/G
5-4: 48385525A/G
5-4: 48414879T/A
5-4: 48453939C/T
5-4: 48496305G/A
5-4: 48526149G/A
5-4: 48551252T/C
5-4: 48571080C/T
5-4: 48674835C/T
5-4: 48706802A/G
5-4: 48758224A/T
5-4: 48812144T/C
5-4: 48902744G/C
5-4: 48951075G/A
5-4: 48962531T/C
5-4: 49136276C/T
5-4: 49144595C/A
5-4: 49156745G/A
5-4: 49194327G/A
5-4: 49220235G/T
5-4: 49269807G/A
5-4: 49356407T/C
5-4: 49370629C/T
5-4: 49415909G/A
5-4: 49476099G/C
5-4: 49512048A/G
5-4: 49578365A/G
5-4: 49653014C/A
5-4: 49720559G/A
5-4: 49762305C/G
5-4: 49789992A/G
5-4: 49859785C/T
5-4: 49926497A/T
5-4: 50020952C/A
5-4: 50035541G/C
5-4: 50077787G/A
5-4: 50218783C/G
5-4: 50235827A/G
5-4: 50273902A/G
5-4: 50367161A/C
5-4: 50419490C/T
5-4: 50419549C/T
5-4: 50430021T/G
5-4: 50470910G/A
5-4: 50516021C/T
5-4: 50532998T/C
5-4: 50563515G/A
5-4: 50606690T/C
5-4: 50619166G/A
5-4: 50630096A/T
5-4: 50674500C/G
5-4: 50686640C/A
5-4: 50706888T/G
5-4: 50728768C/T
5-4: 50787558G/A
5-4: 50892190T/C
5-4: 51028050T/G
5-4: 51124255T/G
5-4: 51142534A/T
5-4: 51172325A/G
5-4: 51183597G/A
5-4: 51183849T/C
5-4: 51184682A/G
5-4: 51213283C/A
5-4: 51247606C/T
5-4: 51291786C/T
5-4: 51328220A/G
5-4: 51342268G/A
5-4: 51396147G/A
5-4: 51420482G/A
5-4: 51435779A/C
5-4: 51495898A/T
5-4: 51548504C/A
5-4: 51618416G/A
5-4: 51743213A/C
5-4: 51750814C/A
5-4: 51767600C/T
5-4: 51792871A/T
5-4: 51804959C/T
5-4: 51834800G/C
5-4: 51862772C/A
5-4: 51928048C/T
5-4: 51949952A/C
5-4: 51966416T/A
5-4: 51978006T/A
5-4: 51993884T/G
5-4: 51994463C/T
5-4: 52010714G/C
5-4: 52045706C/T
5-4: 52052680C/T
5-4: 52074847C/T
5-4: 52129005A/G
5-4: 52179088T/C
5-4: 52213293G/A
5-4: 52249697T/C
5-4: 52285191T/C
5-4: 52329482G/T
5-4: 52374114T/G
5-4: 52422910A/C
5-4: 52458932G/A
5-4: 52497426A/T
5-4: 52545697T/G
5-4: 52573110A/C
5-4: 52645666T/G
5-4: 52661396C/T
5-4: 52686265C/T
5-4: 52698875C/A
5-4: 52712081A/C
5-4: 52755910G/T
5-4: 52797036G/A
5-4: 52818199C/T
5-4: 52831863A/T
5-4: 52846779G/A
5-4: 52876502C/T
5-4: 52886511G/A
5-4: 53084330C/A
5-4: 53187645G/A
5-4: 53225038A/G
5-4: 53322080T/C
5-4: 53354079C/T
5-4: 53476448C/A
5-4: 53492833A/T
5-4: 53508111C/T
5-4: 53553643G/A
5-4: 53686134C/T
5-4: 53697727G/T
5-4: 53773180C/T
5-4: 53793745C/A
5-4: 53804658T/A
5-4: 53825602T/C
5-4: 53870715C/T
5-4: 53899365C/T
5-4: 53952301G/A
5-4: 53981089C/T
5-4: 54012761G/A
5-4: 54052919C/T
5-4: 54072260G/A
5-4: 54082968G/A
5-4: 54118926G/T
5-4: 54130442A/G
5-4: 54152860G/A
5-4: 54156010A/G
5-4: 54156022G/A
5-4: 54156651T/C
5-4: 54175334C/T
5-4: 54187597C/T
5-4: 54216100G/A
5-4: 54231046G/A
5-4: 54241167T/C
5-4: 54267892G/A
5-4: 54304693G/C
5-4: 54333640C/T
5-4: 54413322C/T
5-4: 54616519T/A
5-4: 54632802A/C
5-4: 54696116T/C
5-4: 54713880A/G
5-4: 54738853C/T
5-4: 54820510A/G
5-4: 54866820T/A
5-4: 54908961C/T
5-4: 54931080T/A
5-4: 54960772C/G
5-4: 55014998T/A
5-4: 55042480C/A
5-4: 55097889G/A
5-4: 55147119G/A
5-4: 55189632G/C
5-4: 55267322G/A
5-4: 55311477G/C
5-4: 55405872T/G
5-4: 55432983T/C
5-4: 55530308C/T
5-4: 55556521A/G
5-4: 55556538C/T
5-4: 55657576A/T
5-4: 55699678T/A
5-4: 55701741G/A
5-4: 55712851A/G
5-4: 55731272A/G
5-4: 55736668T/A
5-4: 55753554A/C
5-4: 55765612A/T
5-4: 55856822G/A
5-4: 55974580A/T
5-4: 56093133A/T
5-4: 56094813A/G
5-4: 56112959G/A
5-4: 56113207T/C
5-4: 56144692C/T
5-4: 56200714T/A
5-4: 56272126C/A
5-4: 56288153G/A
5-4: 56339598A/C
5-4: 56404939C/T
5-4: 56447775G/A
5-4: 56460969T/C
5-4: 56480523T/G
5-4: 56509530A/T
5-4: 56635931T/A
5-4: 56691888C/T
5-4: 56735316A/G
5-4: 56749817G/A
5-4: 56775194T/C
5-4: 56970288A/C
5-4: 56972027C/T
5-4: 56972030C/T
5-4: 56972932G/A
5-4: 56973063G/A
5-4: 56979067C/T
5-4: 56980619C/T
5-4: 56981161T/C
5-4: 56983088T/C
5-4: 57071840A/G
5-4: 57132974A/T
5-4: 57176810T/C
5-4: 57205742C/T
5-4: 57217854C/T
5-4: 57230043C/A
5-4: 57257061C/T
5-4: 57300081A/C
5-4: 57310267T/A
5-4: 57355408A/T
5-4: 57446245T/C
5-4: 57496210G/A
5-4: 57593214A/G
5-4: 57608618A/G
5-4: 57664379C/T
5-4: 57670830A/G
5-4: 57671031C/T
5-4: 57731398G/A
5-4: 57749277A/G
5-4: 57773764A/G
5-4: 57820816G/A
5-4: 57891343G/A
5-4: 57934885A/T
5-4: 57982719A/G
5-4: 57984741A/C
5-4: 58042394T/C
5-4: 58081033C/T
5-4: 58121476G/A
5-4: 58183461A/G
5-4: 58197704A/C
5-4: 58206344A/C
5-4: 58228360G/A
5-4: 58271782A/G
5-4: 58300731C/T
5-4: 58351717G/T
5-4: 58378258T/A
5-4: 58404702T/C
5-4: 58421514C/T
5-4: 58505283G/A
5-4: 58535695G/A
5-4: 58537016T/G
5-4: 58568316C/T
5-4: 58568904T/A
5-4: 58586726T/G
5-4: 58590162T/A
5-4: 58650328T/C
5-4: 58661987C/A
5-4: 58687280G/C
5-4: 58715768T/C
5-4: 58751948T/G
5-4: 58817607A/T
5-4: 58853413C/T
5-4: 58867898G/A
5-4: 58873343C/T
5-4: 58873657G/A
5-4: 58873852G/A
5-4: 58878472C/G
5-4: 58972758G/A
5-4: 58973317C/T
5-4: 58982500T/A
5-4: 59012569T/A
5-4: 59041027T/C
5-4: 59079325A/C
5-4: 59096053A/G
5-4: 59098047G/T
5-4: 59184685C/A
5-4: 59275676G/A
5-4: 59319405A/T
5-4: 59323524C/T
5-4: 59343603A/G
5-4: 59348543T/A
5-4: 59349079G/C
5-4: 59349628G/A
5-4: 59383813C/T
5-4: 59408342G/A
5-4: 59457898T/C
5-4: 59485291G/A
5-4: 59534313A/C
5-4: 59546193T/A
5-4: 59643288C/T
5-4: 59738080G/A
5-4: 59815362C/G
5-4: 59817928G/C
5-4: 59821078T/C
5-4: 59864595A/G
5-4: 59877872A/G
5-4: 59885579C/A
5-4: 59908912T/C
5-4: 59941402A/G
5-4: 59996452T/C
5-4: 60029212A/G
5-4: 60034385A/T
5-4: 60094556G/T
5-4: 60131717C/A
5-4: 60190390C/A
5-4: 60257996C/T
5-4: 60268783G/A
5-4: 60315986G/A
5-4: 60348117A/T
5-4: 60435181C/G
5-4: 60513349T/C
5-4: 60540962G/A
5-4: 60606455T/C
5-4: 60647180G/A
5-4: 60700135C/T
5-4: 60769611A/G
5-4: 60836687A/G
5-4: 60887224A/G
5-4: 60901814A/T
5-4: 61001408C/T
5-4: 61044098T/C
5-4: 61079354A/C
5-4: 61116633G/A
5-4: 61146264A/C
5-4: 61172384T/A
5-4: 61215028G/A
5-4: 61283319C/A
5-4: 61319296T/A
5-4: 61328124T/C
5-4: 61430173T/C
5-4: 61451816T/G
5-4: 61474580A/G
5-4: 61610266G/A
5-4: 61658899T/A
5-4: 61671348G/A
5-4: 61744628T/G
5-4: 61791146T/C
5-4: 61860239T/A
5-4: 61866931A/G
5-4: 61949728C/T
5-4: 61983793C/A
5-4: 62014951T/C
5-4: 62067168G/A
5-4: 62160183A/G
5-4: 62204417T/C
5-4: 62251907C/A
5-4: 62296464C/A
5-4: 62341430T/C
5-4: 62376920A/T
5-4: 62581603G/A
5-4: 62695247A/G
5-4: 62706078C/T
5-4: 62749644A/C
5-4: 62780764A/C
5-4: 62833193T/C
5-4: 62839166A/G
5-4: 62879261G/T
5-4: 62899301G/T
5-4: 62899514T/C
5-4: 62899524T/C
5-4: 62927812C/T
5-4: 62937688G/A
5-4: 62938058G/T
5-4: 62941555A/C
5-4: 62978750C/T
5-4: 63017993G/A
5-4: 63028689G/T
5-4: 63063660G/A
5-4: 63070554T/G
5-4: 63102437A/G
5-4: 63131132G/C
5-4: 63178412A/G
5-4: 63190894A/G
5-4: 63225163G/A
5-4: 63260318T/A
5-4: 63297540G/A
5-4: 63339876T/C
5-4: 63428733A/T
5-4: 63436817A/G
5-4: 63444108A/G
5-4: 63467556G/A
5-4: 63486294G/A
5-4: 63544678G/A
5-4: 63557697A/G
5-4: 63569199A/G
5-4: 63587051T/C
5-4: 63601908G/A
5-4: 63633661A/T
5-4: 63682960A/G
5-4: 63717155C/T
5-4: 63794550C/T
5-4: 63830081T/A
5-4: 63865636G/T
5-4: 63890997A/G
5-4: 63933816A/G
5-4: 64047933C/T
5-4: 64055582G/A
5-4: 64069605G/T
5-4: 64083396A/G
5-4: 64101692A/T
5-4: 64120360G/T
5-4: 64232505C/T
5-4: 64237001T/A
5-4: 64335088C/T
5-4: 64500655A/T
5-4: 64587483T/G
5-4: 64602866T/A
5-4: 64627903G/A
5-4: 64663742T/C
5-4: 64715077T/A
5-4: 64745211T/A
5-4: 64762165T/C
5-4: 64811802C/T
5-4: 64841886A/G
5-4: 64855774C/T
5-4: 64874375A/T
5-4: 64911422A/G
5-4: 64966871T/C
5-4: 65026929C/T
5-4: 65076708T/C
5-4: 65135165G/A
5-4: 65301149C/G
5-4: 65407710C/T
5-4: 65439830A/G
5-4: 65481945A/G
5-4: 65532312C/T
5-4: 65604387T/C
5-4: 65634996G/A
5-4: 65698316G/A
5-4: 65720237C/A
5-4: 65720673T/A
5-4: 65732318G/T
5-4: 65774155C/T
5-4: 65776525C/T
5-4: 65822751A/T
5-4: 65900386A/C
5-4: 65913473G/A
5-4: 65929312C/T
5-4: 65954025T/G
5-4: 65989193G/A
5-4: 65996952T/A
5-4: 66015730C/T
5-4: 66050765A/G
5-4: 66138117T/C
5-4: 66199485C/T
5-4: 66252299A/G
5-4: 66285712T/C
5-4: 66303329C/T
5-4: 66318569C/T
5-4: 66416550A/G
5-4: 66439333C/A
5-4: 66463212C/T
5-4: 66502434A/G
5-4: 66763440G/A
5-4: 66955662T/C
5-4: 67037082G/A
5-4: 67086617C/G
5-4: 67188095T/C
5-4: 67192680T/C
5-4: 67267735G/A
5-4: 67387201T/G
5-4: 67535107C/A
5-4: 67646810T/A
5-4: 67661619T/A
5-4: 67848755A/T
5-4: 67867555A/C
5-4: 67890641A/G
5-4: 67991911G/A
5-4: 68143736G/A
5-4: 68173909A/G
5-4: 68195244T/C
5-4: 68221454T/C
5-4: 68248067C/A
5-4: 68339546G/A
5-4: 68500987A/T
5-4: 68610705G/T
5-4: 68617769G/A
5-4: 68621109A/C
5-4: 68632469T/C
5-4: 68762352C/T
5-4: 68866640G/A
5-4: 69052534G/A
5-4: 69056752C/T
5-4: 69074517G/A
5-4: 69132986G/C
5-4: 69147333C/T
5-4: 69200542G/A
5-4: 69313442G/A
5-4: 69756971C/A
5-4: 69878869A/G
5-4: 69965480G/A
6-1: 359528C/G
6-1: 442366C/T
6-1: 586140G/C
6-1: 613285T/A
6-1: 801890T/A
6-1: 912015G/C
6-1: 1014847T/C
6-1: 1323765G/T
6-1: 1424265T/A
6-1: 1481331G/A
6-1: 1542747A/G
6-1: 1581133C/A
6-1: 1591786A/G
6-1: 1592904T/C
6-1: 1647586C/A
6-1: 1712440T/C
6-1: 1757859T/G
6-1: 1802800C/A
6-1: 1978121C/A
6-1: 1978164A/G
6-1: 2008146G/T
6-1: 2095289G/T
6-1: 2199589A/G
6-1: 2281819G/C
6-1: 2327538C/T
6-1: 2448655C/G
6-1: 2477139C/A
6-1: 2555694T/C
6-1: 2636421T/C
6-1: 2677317A/G
6-1: 2689889C/A
6-1: 2749251T/A
6-1: 2790583A/G
6-1: 2826701A/T
6-1: 2852778T/C
6-1: 2895064C/T
6-1: 2910622C/T
6-1: 2959208G/T
6-1: 3047854A/T
6-1: 3051523C/T
6-1: 3122256A/T
6-1: 3152449A/G
6-1: 3233991T/C
6-1: 3316093G/A
6-1: 3348604G/A
6-1: 3401581T/A
6-1: 3420207C/T
6-1: 3498946G/T
6-1: 3542237T/A
6-1: 3560501T/A
6-1: 3654139C/T
6-1: 3665027T/A
6-1: 3730662A/G
6-1: 3768273G/A
6-1: 3791357C/T
6-1: 3809183A/G
6-1: 3859241T/G
6-1: 3884861T/A
6-1: 3913351A/G
6-1: 3965284T/C
6-1: 4018808T/A
6-1: 4052634T/G
6-1: 4097085G/A
6-1: 4127425A/G
6-1: 4171699C/A
6-1: 4182170T/C
6-1: 4207872A/C
6-1: 4248427C/A
6-1: 4303618T/C
6-1: 4343122A/G
6-1: 4366547C/T
6-1: 4424132T/C
6-1: 4433520C/T
6-1: 4438044C/G
6-1: 4439072A/G
6-1: 4460899T/C
6-1: 4479676A/G
6-1: 4479679G/C
6-1: 4649472T/A
6-1: 4689714C/T
6-1: 4713046T/A
6-1: 4776777C/T
6-1: 4826518A/T
6-1: 4846718A/T
6-1: 5007963C/T
6-1: 5027415C/A
6-1: 5142901C/T
6-1: 5168635A/G
6-1: 5297347T/C
6-1: 5337665C/T
6-1: 5359956A/T
6-1: 5424408C/A
6-1: 5455772G/C
6-1: 5507462T/A
6-1: 5605678C/T
6-1: 5709853A/G
6-1: 5723914G/A
6-1: 5755913C/A
6-1: 5816868A/G
6-1: 5847688A/T
6-1: 5977683T/G
6-1: 6015842G/A
6-1: 6016905A/T
6-1: 6057207A/C
6-1: 6070919C/T
6-1: 6096093C/T
6-1: 6180012T/C
6-1: 6187868A/T
6-1: 6188796T/G
6-1: 6190784A/C
6-1: 6191780T/A
6-1: 6206566A/G
6-1: 6207595G/A
6-1: 6221848G/T
6-1: 6254164A/C
6-1: 6294582C/T
6-1: 6335652G/A
6-1: 6450545T/A
6-1: 6461573A/T
6-1: 6469475T/C
6-1: 6469537C/T
6-1: 6471445T/C
6-1: 6472275G/A
6-1: 6484915C/T
6-1: 6500078G/A
6-1: 6541052A/G
6-1: 6607545T/C
6-1: 6638034G/A
6-1: 6673023C/T
6-1: 6700909C/T
6-1: 6745334A/C
6-1: 6771703A/G
6-1: 6798860T/A
6-1: 6943557A/G
6-1: 6976658A/G
6-1: 7032439C/T
6-1: 7136003A/T
6-1: 7203228A/G
6-1: 7273949T/A
6-1: 7285803C/G
6-1: 7353863G/T
6-1: 7497826T/C
6-1: 7561334G/A
6-1: 7609313C/T
6-1: 7695285T/C
6-1: 7706360G/C
6-1: 7742023T/G
6-1: 7784700T/C
6-1: 7864743T/G
6-1: 7953166A/C
6-1: 7989753A/G
6-1: 8002622T/G
6-1: 8005344C/T
6-1: 8008193C/T
6-1: 8013410A/G
6-1: 8127637C/T
6-1: 8170569T/C
6-1: 8220186T/C
6-1: 8228951C/A
6-1: 8231953A/G
6-1: 8298737T/C
6-1: 8345798C/T
6-1: 8410988T/A
6-1: 8440918T/C
6-1: 8451072G/A
6-1: 8477599G/T
6-1: 8512308G/A
6-1: 8575219T/C
6-1: 8616727G/T
6-1: 8660001C/G
6-1: 8750996T/C
6-1: 8762452T/C
6-1: 8780300A/T
6-1: 8790616T/A
6-1: 8841968G/C
6-1: 8889924A/T
6-1: 8906294G/A
6-1: 8991010G/C
6-1: 9070191C/G
6-1: 9076873G/A
6-1: 9187213T/C
6-1: 9203341A/G
6-1: 9295987A/C
6-1: 9377777C/A
6-1: 9431698C/T
6-1: 9451808G/A
6-1: 9452099A/C
6-1: 9524122A/G
6-1: 9548239C/G
6-1: 9633193G/A
6-1: 9678517T/A
6-1: 9731837A/G
6-1: 9935424C/T
6-1: 9956962G/A
6-1: 9986460A/G
6-1: 9997374T/C
6-1: 9997688T/A
6-1: 9998005A/C
6-1: 9998534G/A
6-1: 10000393C/T
6-1: 10001053T/G
6-1: 10002073T/A
6-1: 10110790C/G
6-1: 10179836T/C
6-1: 10247214A/C
6-1: 10251356G/A
6-1: 10370864T/G
6-1: 10372963A/T
6-1: 10543423T/C
6-1: 10554535G/A
6-1: 10589903C/T
6-1: 10630732A/G
6-1: 10673420A/G
6-1: 10673488T/C
6-1: 10687134C/T
6-1: 10706068T/A
6-1: 10789317T/C
6-1: 10816983G/A
6-1: 10841441G/A
6-1: 10898581T/A
6-1: 10908118T/C
6-1: 10908123T/G
6-1: 10908127T/A
6-1: 10908241G/A
6-1: 10908242A/G
6-1: 10908274C/T
6-1: 10953821C/T
6-1: 10955848C/T
6-1: 10971578G/T
6-1: 10971592T/C
6-1: 10971625C/T
6-1: 10971628A/C
6-1: 10971691A/T
6-1: 10971712A/.
6-1: 10971713T/A
6-1: 10971899T/G
6-1: 10971919T/C
6-1: 10971953C/T
6-1: 10971961A/C
6-1: 10971966A/.
6-1: 10994974T/A
6-1: 11040217G/A
6-1: 11107462C/T
6-1: 11142045T/C
6-1: 11207387T/C
6-1: 11255651C/T
6-1: 11296170A/T
6-1: 11329238T/A
6-1: 11329816G/A
6-1: 11340139T/C
6-1: 11388061C/T
6-1: 11398935C/A
6-1: 11420299T/C
6-1: 11421741A/T
6-1: 11422665G/T
6-1: 11422763T/C
6-1: 11423609C/T
6-1: 11423794C/T
6-1: 11424145C/A
6-1: 11424330C/A
6-1: 11626021G/A
6-1: 11634996C/T
6-1: 11663286A/C
6-1: 11703777T/C
6-1: 11764567C/T
6-1: 11797096G/A
6-1: 11810705T/C
6-1: 11855460G/C
6-1: 11927161C/T
6-1: 11959735C/G
6-1: 11972258C/T
6-1: 11974382T/C
6-1: 11992613T/C
6-1: 12135402A/G
6-1: 12149332T/C
6-1: 12169802A/G
6-1: 12181214T/C
6-1: 12231532T/C
6-1: 12273883A/G
6-1: 12276944T/A
6-1: 12278141A/G
6-1: 12278269C/A
6-1: 12295191G/T
6-1: 12340165T/G
6-1: 12438379T/C
6-1: 12542506A/T
6-1: 12607343A/G
6-1: 12635223T/C
6-1: 12657001G/A
6-1: 12702077A/T
6-1: 12763866T/C
6-1: 12820359C/T
6-1: 12885179G/T
6-1: 12945681A/G
6-1: 12955814C/T
6-1: 12958630A/G
6-1: 12988039C/T
6-1: 13015720G/A
6-1: 13036688G/A
6-1: 13107398T/C
6-1: 13119017A/T
6-1: 13137238T/A
6-1: 13153786G/A
6-1: 13157494A/G
6-1: 13159556A/G
6-1: 13167764T/C
6-1: 13194938C/A
6-1: 13197694G/A
6-1: 13224402A/G
6-1: 13256371G/A
6-1: 13268158G/T
6-1: 13294020A/G
6-1: 13319834G/A
6-1: 13332320A/C
6-1: 13411084G/A
6-1: 13438546T/C
6-1: 13448934A/G
6-1: 13466700G/T
6-1: 13484043C/T
6-1: 13519891A/G
6-1: 13568150A/G
6-1: 13603491G/A
6-1: 13664543T/A
6-1: 13691565T/C
6-1: 13789642T/A
6-1: 13823847C/T
6-1: 13879199C/A
6-1: 13920024A/G
6-1: 13945881A/G
6-1: 13959650A/G
6-1: 13969766T/A
6-1: 13981735C/T
6-1: 14003558G/T
6-1: 14011087T/C
6-1: 14021475T/C
6-1: 14048314G/A
6-1: 14050863G/A
6-1: 14050927G/A
6-1: 14100493A/T
6-1: 14159872A/C
6-1: 14166674A/G
6-1: 14189091C/T
6-1: 14189235G/A
6-1: 14219062C/A
6-1: 14341434C/T
6-1: 14392185A/G
6-1: 14413823T/G
6-1: 14438286C/T
6-1: 14455312C/A
6-1: 14492540G/A
6-1: 14519449G/C
6-1: 14530214C/A
6-1: 14590701T/G
6-1: 14636056C/T
6-1: 14675412G/A
6-1: 14710609A/G
6-1: 14751051G/C
6-1: 14782928A/T
6-1: 14814865C/G
6-1: 14846564T/G
6-1: 14866072G/A
6-1: 14878201A/C
6-1: 14900488G/T
6-1: 14941022G/C
6-1: 14997461A/C
6-1: 15010214C/T
6-1: 15010431A/G
6-1: 15056312C/T
6-1: 15122687T/C
6-1: 15149803C/T
6-1: 15162215C/A
6-1: 15171737C/T
6-1: 15174148T/C
6-1: 15186703T/A
6-1: 15199298T/G
6-1: 15209942G/A
6-1: 15243114G/T
6-1: 15249041A/G
6-1: 15249098T/G
6-1: 15334621C/G
6-1: 15345615C/T
6-1: 15356576T/C
6-1: 15391920G/A
6-1: 15423266C/T
6-1: 15494214T/C
6-1: 15520146G/A
6-1: 15542285G/T
6-1: 15556095A/T
6-1: 15595632A/C
6-1: 15610643G/A
6-1: 15635643C/T
6-1: 15648876T/C
6-1: 15673638T/G
6-1: 15695118T/C
6-1: 15782433T/A
6-1: 15857931T/C
6-1: 15858205C/T
6-1: 15858549T/C
6-1: 15859067A/G
6-1: 15863524A/T
6-1: 15863581A/T
6-1: 15876567C/T
6-1: 15902169A/G
6-1: 15920364T/C
6-1: 16024724C/A
6-1: 16169923T/C
6-1: 16195220T/C
6-1: 16235878A/G
6-1: 16245894A/T
6-1: 16259074C/T
6-1: 16310272T/C
6-1: 16313774T/C
6-1: 16321121C/T
6-1: 16366507C/T
6-1: 16369180C/T
6-1: 16381063T/A
6-1: 16382928G/T
6-1: 16400180T/A
6-1: 16480409G/A
6-1: 16494007T/C
6-1: 16567582T/C
6-1: 16579292C/T
6-1: 16601300T/A
6-1: 16657245A/T
6-1: 16676830A/G
6-1: 16714364T/C
6-1: 16752246C/T
6-1: 16772673T/C
6-1: 16786172T/G
6-1: 16813005G/A
6-1: 16822860C/G
6-1: 16856503A/T
6-1: 16868711G/A
6-1: 16868717T/A
6-1: 16900923A/G
6-1: 16916604C/T
6-1: 16929025G/A
6-1: 16957367C/A
6-1: 16974398C/G
6-1: 16995365T/G
6-1: 17050258G/A
6-1: 17063388T/G
6-1: 17078695A/G
6-1: 17090417A/T
6-1: 17097380A/T
6-1: 17130227T/A
6-1: 17132226G/T
6-1: 17162525G/A
6-1: 17173217C/T
6-1: 17224029T/A
6-1: 17239193G/T
6-1: 17259451T/C
6-1: 17299941C/G
6-1: 17366594T/C
6-1: 17390596G/A
6-1: 17429014A/C
6-1: 17487712C/G
6-1: 17541643C/A
6-1: 17639066C/G
6-1: 17656209T/A
6-1: 17667818C/T
6-1: 17703692C/T
6-1: 17716508C/G
6-1: 17727496G/A
6-1: 17738914A/G
6-1: 17761868T/C
6-1: 17774191G/T
6-1: 17795684T/C
6-1: 17806952A/T
6-1: 17818123T/A
6-1: 17848601A/G
6-1: 17897127T/G
6-1: 17942239T/C
6-1: 17969813C/T
6-1: 17987478A/G
6-1: 18032332T/A
6-1: 18045246T/A
6-1: 18078193G/A
6-1: 18094691A/T
6-1: 18104833C/A
6-1: 18147110C/T
6-1: 18170915G/A
6-1: 18187910C/A
6-1: 18224011T/G
6-1: 18253525T/C
6-1: 18297679T/C
6-1: 18342908A/T
6-1: 18397004G/T
6-1: 18422029C/T
6-1: 18453519C/A
6-1: 18468843C/T
6-1: 18484121A/G
6-1: 18495230T/C
6-1: 18496836G/A
6-1: 18537886G/T
6-1: 18550893G/A
6-1: 18572142G/A
6-1: 18588636G/A
6-1: 18638416G/C
6-1: 18726742T/A
6-1: 18792846C/T
6-1: 18839056T/A
6-1: 18864343T/C
6-1: 18873183G/A
6-1: 18876221A/T
6-1: 18908929C/G
6-1: 18995344A/.
6-1: 19005813C/T
6-1: 19020309T/G
6-1: 19024983A/C
6-1: 19025075C/T
6-1: 19025090T/C
6-1: 19025138A/T
6-1: 19025163T/C
6-1: 19025293A/G
6-1: 19025327G/T
6-1: 19025392T/C
6-1: 19025409T/G
6-1: 19025440C/T
6-1: 19025450G/T
6-1: 19025451T/C
6-1: 19025478A/G
6-1: 19025564A/G
6-1: 19025573A/G
6-1: 19025632T/C
6-1: 19025639T/G
6-1: 19025666A/T
6-1: 19025675G/A
6-1: 19025723A/T
6-1: 19025840G/A
6-1: 19025858A/G
6-1: 19025862G/T
6-1: 19025873G/T
6-1: 19025882T/C
6-1: 19025976T/C
6-1: 19025978G/T
6-1: 19026002T/C
6-1: 19026098G/A
6-1: 19026107T/A
6-1: 19026150A/T
6-1: 19026167A/G
6-1: 19026272A/G
6-1: 19026306C/T
6-1: 19026335G/A
6-1: 19026536G/A
6-1: 19026569T/.
6-1: 19027140C/T
6-1: 19027209G/A
6-1: 19027230G/C
6-1: 19027299T/C
6-1: 19027404G/A
6-1: 19027476C/T
6-1: 19027485G/T
6-1: 19027554T/A
6-1: 19027641T/C
6-1: 19027656C/T
6-1: 19027657G/A
6-1: 19027701T/C
6-1: 19027713G/T
6-1: 19027752G/A
6-1: 19047025T/C
6-1: 19070664T/G
6-1: 19082387G/T
6-1: 19110833T/C
6-1: 19132333G/T
6-1: 19151129A/T
6-1: 19161943C/T
6-1: 19177392T/C
6-1: 19185351C/A
6-1: 19190262A/G
6-1: 19200429A/T
6-1: 19207223A/G
6-1: 19213656A/G
6-1: 19218768C/T
6-1: 19236242T/G
6-1: 19254010T/A
6-1: 19307746A/G
6-1: 19308284T/C
6-1: 19308301C/G
6-1: 19312479T/.
6-1: 19316242G/T
6-1: 19316249T/A
6-1: 19317611T/C
6-1: 19317619T/C
6-1: 19317625G/.
6-1: 19317716C/T
6-1: 19317724T/A
6-1: 19317742G/T
6-1: 19351905G/A
6-1: 19428053T/C
6-1: 19442886G/C
6-1: 19443281C/T
6-1: 19443283T/C
6-1: 19450350C/T
6-1: 19575309C/T
6-1: 19601557T/C
6-1: 19629246G/A
6-1: 19684518T/C
6-1: 19709402T/C
6-1: 19743102G/A
6-1: 19754165A/T
6-1: 19755843G/T
6-1: 19811544G/A
6-1: 19877159G/A
6-1: 19928963C/A
6-1: 19967058T/G
6-1: 20016302T/C
6-1: 20151091T/A
6-1: 20254917G/T
6-1: 20266180A/G
6-1: 20278300C/G
6-1: 20317266C/T
6-1: 20332850A/T
6-1: 20343459C/T
6-1: 20343739A/C
6-1: 20366965A/G
6-1: 20373137T/C
6-1: 20383203T/C
6-1: 20415696G/A
6-1: 20439128C/G
6-1: 20464286A/G
6-1: 20493682A/G
6-1: 20567757G/A
6-1: 20776761T/C
6-1: 20928846T/C
6-1: 21028316T/G
6-1: 21205017G/T
6-1: 21288807A/T
6-1: 21338726A/G
6-1: 21367172T/C
6-1: 22159822T/G
6-1: 22408567C/A
6-1: 22410020C/T
6-1: 22422974A/G
6-1: 22441693C/A
6-1: 22452231A/G
6-1: 22463104T/A
6-1: 22470632C/T
6-1: 22473999G/A
6-1: 22481459G/A
6-1: 22502343A/G
6-1: 22518459G/A
6-1: 22534493C/A
6-1: 22544760G/C
6-1: 22555152T/C
6-1: 22690987G/A
6-1: 22826121G/A
6-1: 22830418A/T
6-1: 22894223T/C
6-1: 23151976A/T
6-1: 23342082T/A
6-1: 23717510C/A
6-1: 24496741A/G
6-1: 26165694T/C
6-1: 26676034C/G
6-1: 26851012A/T
6-1: 26986563T/C
6-1: 27063759C/T
6-1: 27183849C/A
6-1: 27301602A/C
6-1: 27665419A/G
6-1: 27681188C/T
6-1: 27681307T/A
6-1: 27681327T/C
6-1: 27692088A/C
6-1: 27693126T/G
6-1: 27704455C/T
6-1: 27859205C/A
6-1: 28014486T/C
6-1: 28036398A/C
6-1: 28290907A/G
6-1: 28297444T/C
6-1: 28321801T/C
6-1: 28379895G/A
6-1: 28644664C/T
6-1: 28669745G/A
6-1: 28713656A/G
6-1: 28939005G/A
6-1: 29197732G/A
6-1: 29236671C/T
6-1: 29266595T/C
6-1: 29307733C/T
6-1: 29365182A/G
6-1: 29456938C/T
6-1: 29590459C/T
6-1: 29716694A/G
6-1: 29786819C/T
6-1: 29984481A/G
6-1: 30109790C/A
6-1: 30159335C/A
6-1: 30186109G/A
6-1: 30227884G/A
6-1: 30440833A/C
6-1: 30656361G/A
6-1: 30693702T/G
6-1: 30710933A/G
6-1: 30726411A/G
6-1: 30782581C/A
6-1: 30845243C/T
6-1: 31039742A/G
6-1: 31132105A/G
6-1: 31224046A/T
6-1: 31401366G/A
6-1: 31427509G/A
6-1: 31436860T/A
6-1: 31437888T/C
6-1: 31437950A/G
6-1: 31455124C/T
6-1: 31460751G/C
6-1: 31487223T/C
6-1: 31600774T/A
6-1: 31650187A/G
6-1: 31656525G/C
6-1: 31693646G/T
6-1: 31704391G/C
6-1: 31705764C/T
6-1: 31706074T/C
6-1: 31706425G/A
6-1: 31709014G/T
6-1: 31709278T/C
6-1: 31710224C/G
6-1: 31721945A/G
6-1: 31764948T/C
6-1: 31789001C/T
6-1: 31794520T/C
6-1: 31796387G/A
6-1: 31800319A/G
6-1: 31806658C/T
6-1: 31809623T/A
6-1: 31811403C/T
6-1: 31811456T/C
6-1: 31811948C/T
6-1: 31812072T/G
6-1: 31823965A/G
6-1: 31846871T/G
6-1: 31860061G/A
6-1: 31867067T/A
6-1: 31871289A/T
6-1: 31873244T/A
6-1: 31874058T/G
6-1: 31874101A/G
6-1: 31887213T/C
6-1: 31900295G/C
6-1: 31956510A/C
6-1: 32028436A/G
6-1: 32076249G/T
6-1: 32086027T/A
6-1: 32086847G/T
6-1: 32103727C/T
6-1: 32123806A/C
6-1: 32134203A/T
6-1: 32292544G/A
6-1: 32338651A/G
6-1: 32379920G/A
6-1: 32398321A/T
6-1: 32409398G/T
6-1: 32431900G/A
6-1: 32464734G/A
6-1: 32501810T/C
6-1: 32513005C/T
6-1: 32550078T/.
6-1: 32578069T/G
6-1: 32728628G/A
6-1: 32747241C/T
6-1: 32791648A/G
6-1: 32839871T/A
6-1: 32947761C/T
6-1: 32963902T/C
6-1: 32991303C/T
6-1: 33055000A/C
6-1: 33185380T/A
6-1: 33190481T/A
6-1: 33238632T/C
6-1: 33286339T/G
6-1: 33287491T/A
6-1: 33336999C/T
6-1: 33339121T/A
6-1: 33339474G/T
6-1: 33390821G/A
6-1: 33470529C/A
6-1: 33544289T/G
6-1: 33586419T/C
6-1: 33622572C/A
6-1: 33667920A/G
6-1: 33740188T/C
6-1: 33876177C/T
6-1: 33953174T/C
6-1: 34139282G/A
6-1: 34162631T/C
6-1: 34193117A/G
6-1: 34225235G/A
6-1: 34349590A/G
6-1: 34411599C/G
6-1: 34442205C/T
6-1: 34538084C/G
6-1: 34623230T/C
6-1: 34644102A/G
6-1: 34657459A/T
6-1: 34686262C/T
6-1: 34777336T/C
6-1: 34814754C/A
6-1: 34858838T/A
6-1: 34878314A/C
6-1: 34914162A/T
6-1: 35224088A/G
6-1: 35276575G/C
6-1: 35354049T/A
6-1: 35722538T/C
6-1: 35844757A/G
6-1: 35857633C/T
6-1: 35942117C/T
6-1: 35994348A/G
6-1: 36125962A/G
6-1: 36132535T/A
6-1: 36190232C/A
6-1: 36259825G/A
6-1: 36288852A/T
6-1: 36288886A/C
6-1: 36289241A/C
6-1: 36289244G/T
6-1: 36289278G/A
6-1: 36289301A/C
6-1: 36289303A/G
6-1: 36289355T/C
6-1: 36289360G/A
6-1: 36289377A/G
6-1: 36289383T/A
6-1: 36289403A/G
6-1: 36289420G/A
6-1: 36289616A/G
6-1: 36291437A/T
6-1: 36291453T/A
6-1: 36291464C/A
6-1: 36291472A/T
6-1: 36291483G/A
6-1: 36291498A/C
6-1: 36291501G/A
6-1: 36291523A/G
6-1: 36291527T/A
6-1: 36291530G/T
6-1: 36291533A/C
6-1: 36291628A/G
6-1: 36291774G/C
6-1: 36291851C/A
6-1: 36291878T/C
6-1: 36291887G/C
6-1: 36291929G/T
6-1: 36291931G/C
6-1: 36292552C/T
6-1: 36365038G/A
6-1: 36493928A/G
6-1: 36513816T/C
6-1: 36632790A/T
6-1: 36639362C/A
6-1: 36642589T/G
6-1: 36643042T/A
6-1: 36682855C/T
6-1: 36759195G/A
6-1: 36782467G/A
6-1: 36826221A/G
6-1: 37046215C/T
6-1: 37156879A/T
6-1: 37204928T/C
6-1: 37349702C/T
6-1: 37395280A/C
6-1: 37442094A/G
6-1: 37523127C/T
6-1: 37576779G/A
6-1: 37635045C/T
6-1: 37637259T/A
6-1: 37647449T/C
6-1: 37657897T/A
6-1: 37659334A/G
6-1: 37698006C/T
6-1: 37736785A/G
6-1: 37750744G/A
6-1: 37762206T/C
6-1: 37778157A/G
6-1: 37778490A/T
6-1: 37782569A/G
6-1: 37805298T/A
6-1: 37833299G/A
6-1: 38104432T/C
6-1: 38234087T/C
6-1: 38262559C/T
6-1: 38289098G/T
6-1: 38324997C/A
6-1: 38340831G/A
6-1: 38355185C/A
6-1: 38374352A/G
6-1: 38385023T/C
6-1: 38537839T/G
6-1: 38558597C/T
6-1: 38600969C/T
6-1: 38817983C/A
6-1: 38817989C/T
6-1: 38828254A/G
6-1: 38832443T/A
6-1: 38883716A/G
6-1: 38929525C/G
6-1: 38971310T/G
6-1: 39027673C/T
6-1: 39127787A/T
6-1: 39164655C/T
6-1: 39281850A/G
6-1: 39380633C/T
6-1: 39549546G/A
6-1: 39583109A/C
6-1: 39621203T/C
6-1: 39672366T/C
6-1: 39862234T/C
6-1: 39960555T/C
6-1: 40026122A/C
6-1: 40246471G/A
6-1: 40298880G/T
6-1: 40483909T/C
6-1: 40592084T/C
6-1: 40742073G/A
6-1: 40827063C/A
6-1: 40893443C/T
6-1: 41084312A/C
6-1: 41317040T/G
6-1: 41346893G/A
6-1: 41349835C/T
6-1: 41392129C/T
6-1: 41431917G/A
6-1: 41461338T/A
6-1: 41473549T/C
6-1: 41520422G/A
6-1: 41534988G/T
6-1: 41593044T/A
6-1: 41652514T/C
6-1: 41661629C/T
6-1: 41664401G/A
6-1: 41723234T/C
6-1: 41840652C/T
6-1: 41861821A/T
6-1: 41872325G/A
6-1: 41907432T/C
6-1: 41907444C/A
6-1: 41972846G/C
6-1: 41990963G/A
6-1: 42022490C/T
6-1: 42064092A/G
6-1: 42154766A/T
6-1: 42166041A/T
6-1: 42176154C/T
6-1: 42188107C/A
6-1: 42263937C/T
6-1: 42282840C/T
6-1: 42341152G/A
6-1: 42369521G/A
6-1: 42381851T/C
6-1: 42474201T/C
6-1: 42520783G/A
6-1: 42608845C/T
6-1: 42646177C/A
6-1: 42731362C/T
6-1: 42913587A/T
6-1: 42943790A/T
6-1: 43002489C/T
6-1: 43007804T/G
6-1: 43070183G/A
6-1: 43100650C/T
6-1: 43119548A/T
6-1: 43178680G/T
6-1: 43257341G/T
6-1: 43303261G/T
6-1: 43319735A/C
6-1: 43334981G/A
6-1: 43343393G/T
6-1: 43388839G/T
6-1: 43443501C/T
6-1: 43476125C/T
6-1: 43598050C/T
6-1: 43735071A/G
6-1: 43736209T/A
6-1: 43748488C/A
6-1: 43859211T/A
6-1: 43909413G/A
6-1: 43910275A/C
6-1: 44050277C/A
6-1: 44086330C/A
6-1: 44169262C/T
6-1: 44256214A/T
6-1: 44310657C/T
6-1: 44361860A/T
6-1: 44480923G/A
6-1: 44517588A/G
6-1: 44551900C/T
6-1: 44628080C/T
6-1: 44685967A/T
6-1: 44699182A/T
6-1: 44736984T/A
6-1: 44747030C/T
6-1: 44838006T/A
6-1: 44930479G/A
6-1: 44992138C/T
6-1: 45027545C/T
6-1: 45056109A/G
6-1: 45237355C/T
6-1: 45263596G/C
6-1: 45336567G/A
6-1: 45373382C/T
6-1: 45426411C/T
6-1: 45465130T/A
6-1: 45531168T/G
6-1: 45543198C/A
6-1: 45562439G/A
6-1: 45581673C/T
6-1: 45586058G/A
6-1: 45586272C/T
6-1: 45615605G/A
6-1: 45672172C/T
6-1: 45860699C/T
6-1: 45901473C/A
6-1: 45909126G/A
6-1: 45941477G/A
6-1: 45971015A/C
6-1: 45975693A/G
6-1: 46083225C/T
6-1: 46137008A/T
6-1: 46159129T/C
6-1: 46171364C/T
6-1: 46198321G/C
6-1: 46211892C/T
6-1: 46270296T/C
6-1: 46287527G/A
6-1: 46291315C/A
6-1: 46291501G/A
6-1: 46343146T/C
6-1: 46360077G/A
6-1: 46467056G/C
6-1: 46483992C/A
6-1: 46507176T/G
6-1: 46517484G/A
6-1: 46522654C/A
6-1: 46527655T/C
6-1: 46538248G/A
6-1: 46548485T/A
6-1: 46644272G/A
6-1: 46666300G/A
6-1: 46737134C/T
6-1: 46800298C/A
6-1: 46871254A/G
6-1: 47009236C/T
6-1: 47137020A/C
6-1: 47148000C/T
6-1: 47167892G/T
6-1: 47227161C/T
6-1: 47325152G/T
6-1: 47375291A/G
6-1: 47403137C/T
6-1: 47429558C/T
6-1: 47442570T/A
6-1: 47499037A/T
6-1: 47582135C/T
6-1: 47669983G/A
6-1: 47703414C/G
6-1: 47736059G/T
6-1: 47748929G/A
6-1: 47773745T/A
6-1: 47797726C/T
6-1: 47813604T/C
6-1: 47844821T/C
6-1: 47880418G/A
6-1: 47890510G/A
6-1: 48010463G/A
6-1: 48098096G/A
6-1: 48118836G/A
6-1: 48146440G/A
6-1: 48160511T/C
6-1: 48175697C/T
6-1: 48187221C/T
6-1: 48198284C/T
6-1: 48223452C/T
6-1: 48266226T/A
6-1: 48307525C/T
6-1: 48317861C/G
6-1: 48320989A/C
6-1: 48321315C/T
6-1: 48323992G/A
6-1: 48324002T/A
6-1: 48454036G/A
6-1: 48471317A/G
6-1: 48478580A/C
6-1: 48526259T/C
6-1: 48554769T/C
6-1: 48609020A/G
6-1: 48677242C/T
6-1: 48689961G/A
6-1: 48747368G/C
6-1: 48765765C/T
6-1: 48785237C/T
6-1: 48785369G/C
6-1: 48785683A/G
6-1: 48796153T/C
6-1: 48884980G/A
6-1: 49006616A/T
6-1: 49012683A/T
6-1: 49049854T/C
6-1: 49059647A/G
6-1: 49060532A/T
6-1: 49060533G/A
6-1: 49123536G/T
6-1: 49136359A/T
6-1: 49208349T/C
6-1: 49291178T/C
6-1: 49345988G/A
6-1: 49355847G/A
6-1: 49414066T/C
6-1: 49425261T/C
6-1: 49460337G/A
6-1: 49470408G/A
6-1: 49488671C/T
6-1: 49504036C/T
6-1: 49517123A/G
6-1: 49532340A/C
6-1: 49542357C/T
6-1: 49654329C/T
6-1: 49686355G/A
6-1: 49702856T/A
6-1: 49723998C/T
6-1: 49764316C/T
6-1: 49768406C/A
6-1: 49796721G/A
6-1: 49900327G/A
6-1: 49906850C/T
6-1: 49917537T/A
6-1: 49942079A/G
6-1: 49955391T/C
6-1: 49977974G/A
6-1: 50076962C/T
6-1: 50103812G/A
6-1: 50133000A/G
6-1: 50150805C/T
6-1: 50182912A/G
6-1: 50279708T/C
6-1: 50327248T/A
6-1: 50377878A/G
6-1: 50420875G/T
6-1: 50464781C/T
6-1: 50502986C/T
6-1: 50593673A/C
6-1: 50612884G/A
6-1: 50670531T/C
6-1: 50795778T/C
6-1: 50798365G/A
6-1: 50807012T/C
6-1: 50920237A/T
6-1: 50948290T/A
6-1: 50959329T/A
6-1: 50971791A/G
6-1: 50987112T/C
6-1: 51084422A/T
6-1: 51205481A/T
6-1: 51322850A/G
6-1: 51436398G/A
6-1: 51547768T/C
6-1: 51619369A/T
6-1: 51661277A/T
6-1: 51719474T/C
6-1: 52048717G/A
6-1: 52087090G/A
6-1: 52135585C/T
6-1: 52165476A/T
6-1: 52165996C/T
6-1: 52198682A/G
6-1: 52206535C/T
6-1: 52219316G/C
6-1: 52241912C/T
6-1: 52260560A/G
6-1: 52320993C/T
6-1: 52366612G/A
6-1: 52393944C/T
6-1: 52427805A/G
6-1: 52461892T/A
6-1: 52473872C/G
6-1: 52484379T/C
6-1: 52514698T/C
6-1: 52528045A/T
6-1: 52549956T/C
6-1: 52604361G/A
6-1: 52623865C/G
6-1: 52657329G/T
6-1: 52667448G/A
6-1: 52691094G/T
6-1: 52756575G/A
6-1: 52785334T/A
6-1: 52794128A/T
6-1: 52870381A/G
6-1: 52903858T/C
6-1: 52916299C/T
6-1: 52916445A/T
6-1: 52919611A/G
6-1: 52928903T/A
6-1: 52937774A/C
6-1: 52938340C/T
6-1: 52939049C/T
6-1: 52950298T/C
6-1: 52951469G/T
6-1: 52952260C/T
6-1: 52952319A/T
6-1: 52961143C/T
6-1: 52961190A/G
6-1: 52992295A/T
6-1: 53032325T/C
6-1: 53112407A/G
6-1: 53147886C/T
6-1: 53295834A/T
6-1: 53333085C/T
6-1: 53366573G/A
6-1: 53410174G/A
6-1: 53452020C/T
6-1: 53480755A/G
6-1: 53568244A/T
6-1: 53599624G/A
6-1: 53658700C/T
6-1: 53679452A/G
6-1: 53785963T/G
6-1: 53850285G/A
6-1: 53888634C/T
6-1: 53936199T/C
6-1: 53959172T/C
6-1: 54060189C/T
6-1: 54114703T/A
6-1: 54253561C/T
6-1: 54462460A/T
6-1: 54501881A/G
6-1: 54515078C/T
6-1: 54530086C/T
6-1: 54550024C/T
6-1: 54634800G/A
6-1: 54658602T/C
6-1: 54931382G/A
6-1: 54946863C/T
6-1: 54957016G/A
6-1: 55034503A/T
6-1: 55060792T/C
6-1: 55186399G/A
6-1: 55439096T/C
6-1: 55477577C/G
6-1: 55565977A/G
6-1: 55651738G/A
6-1: 55786561A/G
6-1: 55964380C/T
6-1: 56174168A/T
6-1: 56289506A/G
6-1: 56299753C/T
6-1: 56340738G/A
6-1: 56436360G/A
6-1: 56482432C/A
6-1: 56518059G/A
6-1: 56593559A/T
6-1: 56654908A/G
6-1: 56655702T/A
6-1: 56845012A/T
6-1: 56971223G/A
6-1: 57173077T/C
6-1: 57185381A/G
6-1: 57221711A/T
6-1: 57373158T/A
6-1: 57439799C/A
6-1: 57572213T/A
6-1: 57598437G/C
6-1: 57655231G/C
6-1: 57768119G/A
6-1: 57833836T/A
6-1: 57934739G/A
6-1: 58006502C/T
6-1: 58156229G/C
6-1: 58288276T/C
6-1: 58414263G/A
6-1: 58562751T/C
6-1: 58781907A/G
6-1: 58911423C/T
6-1: 59003441T/C
6-1: 59314734G/A
6-1: 59423769T/C
6-1: 59605828C/A
6-1: 59784497G/A
6-1: 59824453G/C
6-1: 59997333T/C
6-1: 60068352C/T
6-1: 60124149A/C
6-1: 60142712T/C
6-1: 60156034C/A
6-1: 60212009A/G
6-1: 60237089A/G
6-1: 60240387G/A
6-1: 60247641A/T
6-1: 60352215A/G
6-1: 60689301A/T
6-1: 60831872C/T
6-1: 60990787C/T
6-1: 61151959T/C
6-1: 61300727C/T
6-1: 61548873T/G
6-1: 61603987C/T
6-1: 61647645T/A
6-1: 61676085G/A
6-1: 61813034G/A
6-1: 61865153G/A
6-1: 61905885A/T
6-1: 61929192T/C
6-1: 61992140C/T
6-1: 62221616A/T
6-1: 62261867C/T
6-1: 62281480A/T
6-1: 62315948G/A
6-1: 62326027A/G
6-1: 62428190G/T
6-1: 62441131T/C
6-1: 62496716G/A
6-1: 62499428G/A
6-1: 62507280G/T
6-1: 62555523G/A
6-1: 62598318A/T
6-1: 62762100A/C
6-1: 62805890T/G
6-1: 62866518A/G
6-1: 62953044G/A
6-1: 63081267T/A
6-1: 63088594A/T
6-1: 63247762C/A
6-1: 63252038A/G
6-1: 63424580A/G
6-1: 63435232T/C
6-1: 63444507A/C
6-1: 63446056T/C
6-1: 63451103C/T
6-1: 63460285A/T
6-1: 63553284A/G
6-1: 63782807C/G
6-1: 63785727C/A
6-1: 63796588T/G
6-1: 63797094A/G
6-1: 63797609C/T
6-1: 63805483G/A
6-1: 63815573T/C
6-1: 63884567C/T
6-1: 63982539T/C
6-1: 64068201T/C
6-1: 64171498C/T
6-1: 64193428A/C
6-1: 64205389C/T
6-1: 64241774C/T
6-1: 64262861A/G
6-1: 64274821C/A
6-1: 64361884C/A
6-1: 64378456T/A
6-1: 64402338C/T
6-1: 64413476T/A
6-1: 64456977C/A
6-1: 64549650T/C
6-1: 64636067G/T
6-1: 64681305C/T
6-1: 64808598G/A
6-1: 64815056G/A
6-1: 64826251A/T
6-1: 64845000T/C
6-1: 64856178A/G
6-1: 64860293C/T
6-1: 64868450C/A
6-1: 64902964C/T
6-1: 64939306A/G
6-1: 64949897T/G
6-1: 64987858A/G
6-1: 65023602A/G
6-1: 65041104C/T
6-1: 65055884G/T
6-1: 65069956C/T
6-1: 65088566A/T
6-1: 65109507A/G
6-1: 65128647T/A
6-1: 65140659C/T
6-1: 65150773G/A
6-1: 65245500G/C
6-1: 65246231G/A
6-1: 65261547T/G
6-1: 65286642C/T
6-1: 65360966G/C
6-1: 65371361T/C
6-1: 65537424T/A
6-1: 65566758A/G
6-1: 65606857T/C
6-1: 65618299A/T
6-1: 65647178T/C
6-1: 65659385C/T
6-1: 65710741G/A
6-1: 65727972G/A
6-1: 65795709C/T
6-1: 65929477G/A
6-1: 65944303A/G
6-1: 65984040A/G
6-1: 66013886C/T
6-1: 66033463T/C
6-1: 66052786T/C
6-1: 66064891A/G
6-1: 66073589G/A
6-1: 66075117C/T
6-1: 66075882C/A
6-1: 66076522T/C
6-1: 66097205T/C
6-1: 66184302T/C
6-1: 66223133G/A
6-1: 66276173C/A
6-1: 66304400T/C
6-1: 66339932G/A
6-1: 66382461T/C
6-1: 66402640T/A
6-1: 66425150A/C
6-1: 66528574T/A
6-1: 66530094G/T
6-1: 66586988G/A
6-1: 66621940C/G
6-1: 66648206C/T
6-1: 66678978C/T
6-1: 66703390T/C
6-1: 66706933A/C
6-1: 66734898T/G
6-1: 66748864T/G
6-1: 66763245T/C
6-1: 66787938T/C
6-1: 66804336A/C
6-1: 66823102T/C
6-1: 66837585A/C
6-1: 66849718A/T
6-1: 66852311A/G
6-1: 66876095G/A
6-1: 67048519A/G
6-1: 67058968A/G
6-1: 67082412C/T
6-1: 67106401C/T
6-1: 67116121A/C
6-1: 67116605G/T
6-1: 67149270G/A
6-1: 67167707T/A
6-1: 67190002C/T
6-1: 67251475G/T
6-1: 67294723A/G
6-1: 67298779A/G
6-1: 67347531T/A
6-1: 67528664C/T
6-1: 67577966G/C
6-1: 67603277C/A
6-1: 67623740G/A
6-1: 67729918T/C
6-1: 67748200T/C
6-1: 67763560G/T
6-1: 67801756A/G
6-1: 67857589G/A
6-1: 67957438A/G
6-1: 68098170G/T
6-1: 68165041T/G
6-1: 68267817T/C
6-1: 68453500C/T
6-1: 68621171C/A
6-1: 68722967A/C
6-1: 68731647G/A
6-1: 68810568T/A
6-1: 68884247T/C
6-1: 68955980A/T
6-1: 69104604T/A
6-1: 69106814G/T
6-1: 69125632A/C
6-1: 69147506A/G
6-1: 69178353C/T
6-1: 69256889G/A
6-1: 69289948G/T
6-1: 69380529G/A
6-1: 69393149T/C
6-1: 69403735C/G
6-1: 69427330C/T
6-1: 69492501C/T
6-1: 69615192G/T
6-1: 69627421C/T
6-1: 69714807T/C
6-1: 69824051C/A
6-1: 69843760C/T
6-1: 69858957C/T
6-1: 69884119C/A
6-1: 69896915T/A
6-1: 70012661C/A
6-1: 70064252C/A
6-1: 70115120G/C
6-1: 70143274A/G
6-1: 70160355G/A
6-1: 70172796A/C
6-1: 70403587T/G
6-1: 70431079T/G
6-1: 70457866A/T
6-1: 70568382C/T
6-1: 70606409G/T
6-1: 70685464C/T
6-1: 70705551A/C
6-1: 70741251C/T
6-1: 70778943C/T
6-1: 70791002T/C
6-1: 70817457A/G
6-1: 70898522C/A
6-1: 70909739G/A
6-1: 70925623G/C
6-1: 70998683C/T
6-1: 71029364C/T
6-1: 71071595A/G
6-1: 71167575A/G
6-1: 71184639T/A
6-1: 71211643G/A
6-1: 71216695T/A
6-1: 71235714G/A
6-1: 71288120A/G
6-1: 71427781C/T
6-1: 71462226G/A
6-1: 71467679T/A
6-1: 71473175G/A
6-1: 71541281T/A
6-1: 71567056C/G
6-1: 71590074A/G
6-1: 71593396C/T
6-1: 71600951C/T
6-1: 71611470T/C
6-1: 71644371T/A
6-1: 71682339A/G
6-1: 71697507T/C
6-1: 71718673G/T
6-1: 71740176T/A
6-1: 71827359C/T
6-1: 71863019T/C
6-1: 71910741A/C
6-1: 71967312A/G
6-1: 71979634A/G
6-1: 71980646C/G
6-1: 71990524A/G
6-1: 72036203C/T
6-1: 72054713C/T
6-1: 72140553A/G
6-1: 72257177A/G
6-1: 72282470A/G
6-1: 72374727A/G
6-1: 72434179G/A
6-1: 72525105G/T
6-1: 72579203G/T
6-1: 72628021G/A
6-1: 72676216G/A
6-1: 72701148G/C
6-1: 72738727G/A
6-1: 72784344G/A
6-1: 72861614T/G
6-1: 72880316A/G
6-1: 72961793C/T
6-1: 73010682T/A
6-1: 73037863C/T
6-1: 73049079C/A
6-1: 73114606T/C
6-1: 73115276C/T
6-1: 73142558C/T
6-1: 73253641A/C
6-1: 73274791G/A
6-1: 73364848C/A
6-1: 73394521G/T
6-1: 73447039C/T
6-1: 73462025A/G
6-1: 73505581A/T
6-1: 73552258G/A
6-1: 73622551A/G
6-1: 73650167C/A
6-1: 73667568A/G
6-1: 73690784G/T
6-1: 73789869G/A
6-1: 73803032T/A
6-1: 74056604C/G
6-1: 74181775C/T
6-1: 74199957A/G
6-1: 74218845T/C
6-1: 74304049T/C
6-1: 74305089T/C
6-1: 74329420G/T
6-1: 74335831C/A
6-1: 74335855A/C
6-1: 74340378A/T
6-1: 74340899A/G
6-1: 74355294G/A
6-1: 74417811T/C
6-1: 74436872A/G
6-1: 74449665C/T
6-1: 74465114A/C
6-1: 74480577C/T
6-1: 74549106G/A
6-1: 74585730T/C
6-1: 74668353A/T
6-1: 74689909T/C
6-1: 74718406A/G
6-1: 74770937G/A
6-1: 74816425A/G
6-1: 74879443T/C
6-1: 74888263G/A
6-1: 74931893C/G
6-1: 74944040A/T
6-1: 74955954A/T
6-1: 74966845T/C
6-1: 75002346G/A
6-1: 75074227A/C
6-1: 75153048C/T
6-1: 75374710A/G
6-1: 75376384T/C
6-1: 75387323T/C
6-1: 75512512C/T
6-1: 75751328C/T
6-1: 75756366G/A
6-1: 75814529C/T
6-1: 75868148G/A
6-1: 75900688G/T
6-1: 75983876G/T
6-1: 75993882T/A
6-1: 76098818C/G
6-1: 76126411G/A
6-1: 76176505A/G
6-1: 76242167A/G
6-1: 76277528T/A
6-1: 76333245G/A
6-1: 76363182A/G
6-1: 76390766C/A
6-1: 76507479C/G
6-1: 76587587G/A
6-1: 76703078G/A
6-1: 76946547C/T
6-1: 77421631G/T
6-1: 77421651A/T
6-1: 77421676C/T
6-1: 77422072G/A
6-1: 77422075A/G
6-1: 77422147A/G
6-1: 77422213G/A
6-1: 77422776T/A
6-1: 77425232A/G
6-1: 77425253G/A
6-1: 77425276G/T
6-1: 77425287G/T
6-1: 77425304G/A
6-1: 77425310G/T
6-1: 77425337A/T
6-1: 77445895G/A
6-1: 77524856A/C
6-1: 77530627G/A
6-1: 77598844C/A
6-1: 77718313G/A
6-1: 77721844G/A
6-1: 77844637G/A
6-1: 77897561A/T
6-1: 77989299T/C
6-1: 78118658C/T
6-1: 78153983T/G
6-1: 78164719A/T
6-1: 78221523G/A
6-1: 78251883C/A
6-1: 78496930C/T
6-1: 78556536C/T
6-1: 78597063T/C
6-1: 78598884A/T
6-1: 78661109T/G
6-1: 78734914G/C
6-1: 78804978T/G
6-1: 78833084A/T
6-1: 78845385A/G
6-1: 78847341T/A
6-1: 78872927G/A
6-1: 78902997T/C
6-1: 78923764A/T
6-1: 78938405C/T
6-1: 79023069T/G
6-1: 79055776C/T
6-1: 79101854A/G
6-1: 79136500A/G
6-1: 79181972C/A
6-1: 79223966T/C
6-1: 79295076T/A
6-1: 79314886C/T
6-1: 79489546G/A
6-1: 79504683G/A
6-1: 79592028A/G
6-1: 79645901G/A
6-1: 79832748C/A
6-1: 79944115T/C
6-1: 79981191G/T
6-1: 80014004G/A
6-1: 80073108C/T
6-1: 80106587A/G
6-1: 80138791T/A
6-1: 80192974A/C
6-1: 80270643T/C
6-1: 80293835T/C
6-1: 80323212T/G
6-1: 80365007A/G
6-1: 80392106C/T
6-1: 80411004G/C
6-1: 80448220G/A
6-1: 80478017C/A
6-1: 80505091T/G
6-1: 80516927T/C
6-1: 80576517C/T
6-1: 80625620T/C
6-1: 80665077C/A
6-1: 80733760A/T
6-1: 80773194A/C
6-1: 80785560C/T
6-1: 80799765A/T
6-1: 80811239T/C
6-1: 80859964C/A
6-1: 80928031C/T
6-1: 80948647A/T
6-1: 81005683A/G
6-1: 81068334A/T
6-1: 81074069C/T
6-1: 81087369T/C
6-1: 81111816A/G
6-1: 81145423A/G
6-1: 81191030C/T
6-1: 81283427G/T
6-1: 81310678C/A
6-1: 81334527C/G
6-1: 81335116G/A
6-1: 81335158G/A
6-1: 81515944A/C
6-1: 81516848T/A
6-1: 81528097C/T
6-1: 81540731C/A
6-1: 81542472G/A
6-1: 81553129C/A
6-1: 81564414C/T
6-1: 81571567G/A
6-1: 81575360T/C
6-1: 81590367C/T
6-1: 81628241T/C
6-1: 81640862T/C
6-1: 81655094T/C
6-1: 81670043T/C
6-1: 81703191G/C
6-1: 81743511G/A
6-1: 81796483T/C
6-1: 81869651T/C
6-1: 81896158A/T
6-1: 81910800C/T
6-1: 82033146A/G
6-1: 82125903T/C
6-1: 82137999G/A
6-1: 82154017C/T
6-1: 82184313A/G
6-1: 82247125G/A
6-1: 82388116A/G
6-1: 82480497T/G
6-1: 82578788A/C
6-1: 82729109T/C
6-1: 82968471T/C
6-1: 82982889A/T
6-1: 83011682A/T
6-1: 83065345T/A
6-1: 83090706C/G
6-1: 83113965G/A
6-1: 83135418C/T
6-1: 83146671C/T
6-1: 83226446A/G
6-1: 83226795C/T
6-1: 83230540C/A
6-1: 83245134G/A
6-1: 83270982T/G
6-1: 83281084T/C
6-1: 83312390A/T
6-1: 83338193G/A
6-1: 83353224A/T
6-1: 83354137C/A
6-1: 83354220C/T
6-1: 83398927T/C
6-1: 83634636G/A
6-1: 83647010G/A
6-1: 83679245C/T
6-1: 83732756G/C
6-1: 83803851T/A
6-1: 83829502T/G
6-1: 83875109A/G
6-1: 83918797G/A
6-1: 83934624G/A
6-1: 83954729G/A
6-1: 83954734G/A
6-1: 83974925C/A
6-1: 84229133T/G
6-1: 84337048T/C
6-1: 84471625C/A
6-1: 84485216A/C
6-1: 84498541A/G
6-1: 84532808C/T
6-1: 84629431C/T
6-1: 84663093G/A
6-1: 84698543A/T
6-1: 84768723G/A
6-1: 84785413G/A
6-1: 84803401A/G
6-1: 84815307A/T
6-1: 84833160C/T
6-1: 84899038C/G
6-1: 84968631A/C
6-1: 85021964A/G
6-1: 85099371A/G
6-1: 85122163C/T
6-1: 85334216T/A
6-1: 85373238A/G
6-1: 85467255G/T
6-1: 85563642G/T
6-1: 85590417C/G
6-1: 85651553T/C
6-1: 85715219T/G
6-1: 85748860T/G
6-1: 85779091A/G
6-1: 85799496C/T
6-1: 85832131A/G
6-1: 85842841A/C
6-1: 85888641G/C
6-1: 85911395T/C
6-1: 85922542T/C
6-1: 85966834T/C
6-1: 85978432A/G
6-1: 85993483C/T
6-1: 86014041C/T
6-1: 86053755T/A
6-1: 86063316T/G
6-1: 86099848G/A
6-1: 86141566A/G
6-1: 86174417A/T
6-1: 86229034A/G
6-1: 86259656G/A
6-1: 86336418G/A
6-1: 86422213A/C
6-1: 86439712T/G
6-1: 86458952T/C
6-1: 86482955T/C
6-1: 86504188A/G
6-1: 86515723C/A
6-1: 86530753G/A
6-1: 86562477T/A
6-1: 86606279C/T
6-1: 86670662G/T
6-1: 86721813C/T
6-1: 86738919C/T
6-1: 86773799T/C
6-1: 86858038C/T
6-1: 86964846A/G
6-1: 87131197C/A
6-1: 87189396T/C
6-1: 87274615C/T
6-1: 87303444G/A
6-1: 87343128A/G
6-1: 87366145G/C
6-1: 87413607G/T
6-1: 87426687C/G
6-1: 87455432T/C
6-1: 87481445T/G
6-1: 87554195A/G
6-1: 87605533G/A
6-1: 87622191T/C
6-1: 87696182T/C
6-1: 87697189T/C
6-1: 87697202C/T
6-1: 87745680G/A
6-1: 87746185T/C
6-1: 87765377A/C
6-1: 87796589T/C
6-1: 87817473C/T
6-1: 87853673T/C
6-1: 87917079C/A
6-1: 87927290A/G
6-1: 87992758T/G
6-1: 88017184A/G
6-1: 88049192G/A
6-1: 88049807C/T
6-1: 88056139A/G
6-1: 88056154G/T
6-1: 88061244G/A
6-1: 88062305G/A
6-1: 88071468C/A
6-1: 88071974G/A
6-1: 88072241C/T
6-1: 88074880A/G
6-1: 88079376T/C
6-1: 88104025A/G
6-1: 88138387C/T
6-1: 88154756T/G
6-1: 88170910A/T
6-1: 88256572T/C
6-1: 88283090T/C
6-1: 88320755A/T
6-1: 88360299T/G
6-1: 88444324C/T
6-1: 88544184C/T
6-1: 88572886G/A
6-1: 88606609A/G
6-1: 88616560G/A
6-1: 88617990A/G
6-1: 88630182C/A
6-1: 88672860G/A
6-1: 88693047G/A
6-1: 88705386A/G
6-1: 88773027A/G
6-1: 88785461A/G
6-1: 88796643G/C
6-1: 88889643C/T
6-1: 89008969C/A
6-1: 89131185T/G
6-1: 89156413T/C
6-1: 89180088A/T
6-1: 89185339C/A
6-1: 89185719T/G
6-1: 89198292A/C
6-1: 89219273G/A
6-1: 89245564G/A
6-1: 89286752T/C
6-1: 89336416A/C
6-1: 89402604A/C
6-1: 89458743C/A
6-1: 89494313A/C
6-1: 89509187C/T
6-1: 89571154G/A
6-1: 89636903A/T
6-1: 89651604A/G
6-1: 89686630G/A
6-1: 89749700C/T
6-1: 89750505G/A
6-1: 89784488T/A
6-1: 89803306C/T
6-1: 89852852A/T
6-1: 89862826C/T
6-1: 89863078T/C
6-1: 89874501A/T
6-1: 89925481T/C
6-1: 89978676A/G
6-1: 89992064T/C
6-1: 90079623C/T
6-1: 90108276T/C
6-1: 90137232C/G
6-1: 90176315T/A
6-1: 90208093C/G
6-1: 90239888C/T
6-1: 90262875C/T
6-1: 90328239T/A
6-1: 90404247T/C
6-1: 90463996T/C
6-1: 90535678T/A
6-1: 90558315G/T
6-1: 90580763A/G
6-1: 90581242A/G
6-1: 90595234T/A
6-1: 90615908A/G
6-1: 90687488G/A
6-1: 90718551A/C
6-1: 90719110G/A
6-1: 90755974T/G
6-1: 90822924T/C
6-1: 91037272A/T
6-1: 91051213G/C
6-1: 91253826A/T
6-1: 91291949T/C
6-1: 91335546A/G
6-1: 91353851A/G
6-1: 91389670A/G
6-1: 91423715T/C
6-1: 91453458G/A
6-1: 91537472G/A
6-1: 91577694G/C
6-1: 91624811A/G
6-1: 91666176T/C
6-1: 91761818C/A
6-1: 91772760A/C
6-1: 91894170G/A
6-1: 91921418T/C
6-1: 92049969G/A
6-1: 92115341T/C
6-1: 92176408T/A
6-1: 92195625A/T
6-1: 92234761C/T
6-1: 92292832T/C
6-1: 92368668C/T
6-1: 92485493A/T
6-1: 92524607C/A
6-1: 92552752G/A
6-1: 92619296G/A
6-1: 92634490A/G
6-1: 92766390C/T
6-1: 92879351G/A
6-1: 92901071T/A
6-1: 93048323A/T
6-1: 93062487A/T
6-1: 93078516A/T
6-1: 93091481A/G
6-1: 93095747G/A
6-1: 93150637A/T
6-1: 93226491T/G
6-1: 93342934G/C
6-1: 93407340A/G
6-1: 93450596G/C
6-1: 93510684A/T
6-1: 93574220A/G
6-1: 93613663A/C
6-1: 93625220A/G
6-1: 93684559T/C
6-1: 93735352T/C
6-1: 93759094T/A
6-1: 93776148T/C
6-1: 93777987T/C
6-1: 93784132A/G
6-1: 93787284A/G
6-1: 93832660G/T
6-1: 93951161A/G
6-1: 93960144G/T
6-1: 93985645C/T
6-1: 93986165G/T
6-1: 94073260A/G
6-1: 94093992T/C
6-1: 94147899G/A
6-1: 94160819C/T
6-1: 94172463A/G
6-1: 94184285C/T
6-1: 94205789C/T
6-1: 94240683G/A
6-1: 94243931T/C
6-1: 94251646C/T
6-1: 94287600G/A
6-1: 94370687G/A
6-1: 94372318T/C
6-1: 94373309C/A
6-1: 94373427C/T
6-1: 94412526T/G
6-1: 94488979G/A
6-1: 94490381G/A
6-1: 94543196C/T
6-1: 94560163T/C
6-1: 94592593A/G
6-1: 94675485A/G
6-1: 94754385A/G
6-1: 94768266A/T
6-1: 94797776A/G
6-1: 94820263A/G
6-1: 94834127A/C
6-1: 94866831A/G
6-1: 94887970T/C
6-1: 94919460T/C
6-1: 94983459G/A
6-1: 95021444A/G
6-1: 95071399A/G
6-1: 95092511G/A
6-1: 95113295G/A
6-1: 95135178G/A
6-1: 95212911A/G
6-1: 95238099C/T
6-1: 95261632A/G
6-1: 95296021T/C
6-1: 95324053G/T
6-1: 95324428G/A
6-1: 95378816C/T
6-1: 95428663T/C
6-1: 95483022A/G
6-1: 95500908C/A
6-1: 95545938T/C
6-1: 95573883C/T
6-1: 95621417G/T
6-1: 95705777T/C
6-1: 95734837T/A
6-1: 95756232G/A
6-1: 95760553G/C
6-1: 95790905A/G
6-1: 95829291G/A
6-1: 95846955T/C
6-1: 95854547A/C
6-1: 95886640T/A
6-1: 95934499G/C
6-1: 96014215G/A
6-1: 96053128A/G
6-1: 96115241T/A
6-1: 96188459T/A
6-1: 96224448A/G
6-1: 96244129C/G
6-1: 96292378G/A
6-1: 96292913G/A
6-1: 96305471C/T
6-1: 96317407C/A
6-1: 96386654T/G
6-1: 96416567A/C
6-1: 96442052A/T
6-1: 96456284C/T
6-1: 96484417G/A
6-1: 96500203T/C
6-1: 96511295C/A
6-1: 96521331T/A
6-1: 96558436C/T
6-1: 96693924G/A
6-1: 96751824G/A
6-1: 96831297C/T
6-1: 96853870C/T
6-1: 96858093T/C
6-1: 96858168C/G
6-1: 96865251T/C
6-1: 96870439A/C
6-1: 96903532A/G
6-1: 96946714A/G
6-1: 96958144A/C
6-1: 97003928A/T
6-1: 97036578T/A
6-1: 97086223T/C
6-1: 97243879G/A
6-1: 97407542A/T
6-1: 97408746G/A
6-1: 97448907A/C
6-1: 97510408T/C
6-1: 97539165A/G
6-1: 97619778T/C
6-1: 97647149G/A
6-1: 97672729C/G
6-1: 97704235C/A
6-1: 97801816G/A
6-1: 97886351C/A
6-1: 97891314C/T
6-1: 97920236A/G
6-1: 97942131A/G
6-1: 98058065A/G
6-1: 98085051T/A
6-1: 98176626C/T
6-1: 98231553G/A
6-1: 98231975C/T
6-1: 98243094T/A
6-1: 98258598A/G
6-1: 98404926A/G
6-1: 98416294G/A
6-1: 98447110C/T
6-1: 98464002G/A
6-1: 98475277C/T
6-1: 98608073C/G
6-1: 98650437A/G
6-1: 98727139G/A
6-1: 98782970C/G
6-1: 98813501G/A
6-1: 98988095A/G
6-1: 99061583C/T
6-1: 99127117T/C
6-1: 99183375G/A
6-1: 99255619A/G
6-1: 99548729G/A
6-1: 99851436G/A
6-1: 99973576T/A
6-1: 100047621T/A
6-1: 100099845A/G
6-1: 100250345A/T
6-1: 100316232G/A
6-1: 100405665T/G
6-1: 100416617A/G
6-1: 100465045A/G
6-1: 100646018G/A
6-1: 100676766G/T
6-1: 100804147T/C
6-1: 100804468A/G
6-1: 100804523G/T
6-1: 100814739G/C
6-1: 100834975A/G
6-1: 100852880C/G
6-1: 101011223A/G
6-1: 101012301A/G
6-1: 101012314C/T
6-1: 101032197C/T
6-1: 101051387T/C
6-1: 102455279C/T
6-1: 102515128T/G
6-1: 102868462G/A
6-2: 15876T/C
6-2: 43613G/A
6-2: 43688T/C
6-2: 91207C/T
6-2: 116605T/A
6-2: 134318C/T
6-2: 154053T/C
6-2: 192434C/T
6-2: 204601C/A
6-2: 214817G/A
6-2: 301654C/A
6-2: 318610C/T
6-2: 335023G/A
6-2: 352631T/G
6-2: 367113T/C
6-2: 392163C/T
6-2: 423872A/G
6-2: 451490A/T
6-2: 479599G/A
6-2: 501443G/A
6-2: 530857T/C
6-2: 592558T/C
6-2: 695327T/G
6-2: 740489T/A
6-2: 742344A/G
6-2: 775090A/G
6-2: 973313T/A
6-2: 1063526C/A
6-2: 1172971G/A
6-2: 1292310T/G
6-2: 1332682T/C
6-2: 1365782C/A
6-2: 1414089T/C
6-2: 1504061T/A
6-2: 1573584A/T
6-2: 1597621C/G
6-2: 1622034A/T
6-2: 1696733G/T
6-2: 1721734G/C
6-2: 1825128C/T
6-2: 1825142C/T
6-2: 1825154G/T
6-2: 1885248C/T
6-2: 1927576G/A
6-2: 1939089A/G
6-2: 2048652T/G
6-2: 2062721A/G
6-2: 2076083A/T
6-2: 2137110T/C
6-2: 2192337G/A
6-2: 2278146T/G
6-2: 2350727C/T
6-2: 2380169T/C
6-2: 2412371G/T
6-2: 2432338T/A
6-2: 2480069G/A
6-2: 2518042T/C
6-2: 2652790C/T
6-2: 2722585G/T
6-2: 2764837C/T
6-2: 2820616T/C
6-2: 2854674A/G
6-2: 3015910A/G
6-2: 3068881C/T
6-2: 3149762G/C
6-2: 3203570T/C
6-2: 3216621A/T
6-2: 3417100A/G
6-2: 3463270A/G
6-2: 3554431A/G
6-2: 3623812G/A
6-2: 3635784A/G
6-2: 3696347T/A
6-2: 3749206G/A
6-2: 3797996C/T
6-2: 3861608C/T
6-2: 3909312G/T
6-2: 3941631A/T
6-2: 3981182C/T
6-2: 3992697T/G
6-2: 4080004G/A
6-2: 4139351T/C
6-2: 4202052T/C
6-2: 4362883A/C
6-2: 4414197C/A
6-2: 4431767T/G
6-2: 4459857T/C
6-2: 4472569T/C
6-2: 4486889T/A
6-2: 4509921T/C
6-2: 4532072T/C
6-2: 4604299C/T
6-2: 4623121A/G
6-2: 4647623A/G
6-2: 4681598G/A
6-2: 4697702C/T
6-2: 4712427C/G
6-2: 4712536A/G
6-2: 4783611T/G
6-2: 4875256G/A
6-2: 4888540T/A
6-2: 4907044C/G
6-2: 4946850G/A
6-2: 5002659T/A
6-2: 5019249A/T
6-2: 5097491A/C
6-2: 5179750T/A
6-2: 5198179C/T
6-2: 5201877G/A
6-2: 5213231T/C
6-2: 5261330C/A
6-2: 5287890A/G
6-2: 5313151C/G
6-2: 5372009C/T
6-2: 5386361G/A
6-2: 5421883T/C
6-2: 5435140G/A
6-2: 5450555A/G
6-2: 5478140A/G
6-2: 5584360G/A
6-2: 5739175T/C
6-2: 5752217C/T
6-2: 5781725A/T
6-2: 5793696C/T
6-2: 5802638C/A
6-2: 5811444G/T
6-2: 5901456A/G
6-2: 5918067C/T
6-2: 5969646C/T
6-2: 6001167G/A
6-2: 6043856G/T
6-2: 6145029G/T
6-2: 6149040A/T
6-2: 6207818T/A
6-2: 6301508A/G
6-2: 6375222C/T
6-2: 6389035A/T
6-2: 6432327G/A
6-2: 6444917A/T
6-2: 6482251G/A
6-2: 6498795A/T
6-2: 6507132T/C
6-2: 6522190A/G
6-2: 6523597T/A
6-2: 6556250T/C
6-2: 6708654C/T
6-2: 6840210G/A
6-2: 6920985C/A
6-2: 6953835A/G
6-2: 8752740G/A
6-2: 9093201T/A
6-2: 9277870T/C
6-2: 9474895T/C
6-2: 10045552A/T
6-2: 10067958C/T
6-2: 10081234G/A
6-2: 10124900T/G
6-2: 10248680T/C
6-2: 10262659A/T
6-2: 10262773C/T
6-2: 10262787T/C
6-2: 10262796C/T
6-2: 10262910T/C
6-2: 10708576T/A
6-2: 10741689C/T
6-2: 10753858T/C
6-2: 10815737G/A
6-2: 11172857A/G
6-2: 11539283C/A
6-2: 11791010C/T
6-2: 11895517A/G
6-2: 11968005A/G
6-2: 11978933T/C
6-2: 12002645C/G
6-2: 12037630C/T
6-2: 12481195T/C
6-2: 12707290G/C
6-2: 12716515G/A
6-2: 12732669C/T
6-2: 12758496G/A
6-2: 12796567T/C
6-2: 12928284C/T
6-2: 13084266T/C
6-2: 13093199A/G
6-2: 13247334C/T
6-2: 13257359A/G
6-2: 13287955C/T
6-2: 13314240T/A
6-2: 13344092C/T
6-2: 13355098T/C
6-2: 13364903T/C
6-2: 13384465T/A
6-2: 13397876C/T
6-2: 13398612G/A
6-2: 13409076C/A
6-2: 13411063A/T
6-2: 13411309T/C
6-2: 13451704A/C
6-2: 13461938C/T
6-2: 13487531A/T
6-2: 13516249A/G
6-2: 13587627T/C
6-2: 13684596G/C
6-2: 13703690G/A
6-2: 13801558C/T
6-2: 13812510A/G
6-2: 13965260C/T
6-2: 14130211T/A
6-2: 14135958T/C
6-2: 14174973T/A
6-2: 14185505G/C
6-2: 14292826G/A
6-2: 14331074T/A
6-2: 14499295G/T
6-2: 14532146A/C
6-2: 14603898T/G
6-2: 14614188T/C
6-2: 14616008G/T
6-2: 14669098T/C
6-2: 14700675G/A
6-2: 14730802A/C
6-2: 14808879A/G
6-2: 14926533G/A
6-2: 14948992T/C
6-2: 14988926A/G
6-2: 15014670G/A
6-2: 15043361T/C
6-2: 15075083T/C
6-2: 15089588C/T
6-2: 15101279G/A
6-2: 15293481T/C
6-2: 15360277G/A
6-2: 15375247G/A
6-2: 15388490T/A
6-2: 15404563C/T
6-2: 15414811G/T
6-2: 15455858A/G
6-2: 15459721T/G
6-2: 15502677C/T
6-2: 15539592C/T
6-2: 15590024T/C
6-2: 15649489G/T
6-2: 15676713A/G
6-2: 15736061A/T
6-2: 15778362G/T
6-2: 15812364A/C
6-2: 15925790A/G
6-2: 15943641G/A
6-2: 15958657C/T
6-2: 15981123T/A
6-2: 15993839G/A
6-2: 15994167T/C
6-2: 16016040T/C
6-2: 16054741A/G
6-2: 16068693T/C
6-2: 16152026T/G
6-2: 16459637A/G
6-2: 16461286C/A
6-2: 16487653C/T
6-2: 16515641A/G
6-2: 16596524T/A
6-2: 16665358A/G
6-2: 16665990A/T
6-2: 16676051G/A
6-2: 16761859T/G
6-2: 16787669T/C
6-2: 16865124T/G
6-2: 17076275G/A
6-2: 17196739G/A
6-2: 17281302A/C
6-2: 17309554C/T
6-2: 17343064G/T
6-2: 17460982C/T
6-2: 17485423A/T
6-2: 17499299C/G
6-2: 17565207A/T
6-2: 17631821G/A
6-2: 17641946T/C
6-2: 17653244C/T
6-2: 17671194C/T
6-2: 17711334A/G
6-2: 17803201T/A
6-2: 17851950T/C
6-2: 18095963C/T
6-2: 18107581C/T
6-2: 18107706T/A
6-2: 18108372A/T
6-2: 18118485A/C
6-2: 18126913C/T
6-2: 18128068G/T
6-2: 18129129T/C
6-2: 18129135T/C
6-2: 18213529T/C
6-2: 18302535C/T
6-2: 18323016G/A
6-2: 18466008C/T
6-2: 18491911G/A
6-2: 18544604G/T
6-2: 18580463G/A
6-2: 18682753G/A
6-2: 18791342G/A
6-2: 18801755C/T
6-2: 18893397C/A
6-2: 18921311T/G
6-2: 18931860T/A
6-2: 19000128T/A
6-2: 19027629C/T
6-2: 19082838G/A
6-2: 19111436T/C
6-2: 19241438T/C
6-2: 19266495A/T
6-2: 19304405G/A
6-2: 19319047T/C
6-2: 19334918C/T
6-2: 19371498A/G
6-2: 19444235A/G
6-2: 19526064A/C
6-2: 19541540C/A
6-2: 19575171C/T
6-2: 19628572G/A
6-2: 19708554A/G
6-2: 19764274A/G
6-2: 19828134C/T
6-2: 19902410T/C
6-2: 19942232C/A
6-2: 19956499T/A
6-2: 19971732A/C
6-2: 20019023C/T
6-2: 20148456C/T
6-2: 20242327T/C
6-2: 20307490G/A
6-2: 20363262T/C
6-2: 20490739A/G
6-2: 20584742A/G
6-2: 20712663G/C
6-2: 20770605T/G
6-2: 20874728C/G
6-2: 20886791G/A
6-2: 20925805A/G
6-2: 21000647T/A
6-2: 21059925A/T
6-2: 21084892A/T
6-2: 21214252A/T
6-2: 21411234C/T
6-2: 21485193G/T
6-2: 21578750C/T
6-2: 21639417C/T
6-2: 21643851A/C
6-2: 21678731G/A
6-2: 21694203T/A
6-2: 21707645G/A
6-2: 21723293A/G
6-2: 21837338T/C
6-2: 21874948A/G
6-2: 22052740A/G
6-2: 22063007A/T
6-2: 22113866C/T
6-2: 22137762C/T
6-2: 22152379G/A
6-2: 22189834A/G
6-2: 22220558A/G
6-2: 22254786T/C
6-2: 22300028G/T
6-2: 22312750C/T
6-2: 22354406T/G
6-2: 22399824G/A
6-2: 22487558G/C
6-2: 22550385T/C
6-2: 22643653A/G
6-2: 22670443C/T
6-2: 22709663T/C
6-2: 22771785T/A
6-2: 22827901C/G
6-2: 22855034C/A
6-2: 22889581C/A
6-2: 22899583G/A
6-2: 22961228G/A
6-2: 23008543C/T
6-2: 23067810T/C
6-2: 23183701G/A
6-2: 23246615G/T
6-2: 23285112C/T
6-2: 23301063C/T
6-2: 23325583G/T
6-2: 23353016C/T
6-2: 23389557G/C
6-2: 23399097G/A
6-2: 23400496T/C
6-2: 23412046A/T
6-2: 23518155G/C
6-2: 23587836C/A
6-2: 23619376G/C
6-2: 23639006T/A
6-2: 23704312A/G
6-2: 23721644A/G
6-2: 23733312G/A
6-2: 23755865T/C
6-2: 23757718G/A
6-2: 23770848T/C
6-2: 23786052T/A
6-2: 23814345C/T
6-2: 23850265T/G
6-2: 23933744C/A
6-2: 23947749A/T
6-2: 23959742C/T
6-2: 24071371T/A
6-2: 24101202A/T
6-2: 24121576C/T
6-2: 24150174C/T
6-2: 24172167C/A
6-2: 24190575T/A
6-2: 24227018T/C
6-2: 24253183G/A
6-2: 24300715T/A
6-2: 24305662C/T
6-2: 24313257A/G
6-2: 24326429T/C
6-2: 24352067C/A
6-2: 24385121C/T
6-2: 24398956C/T
6-2: 24415328T/C
6-2: 24454684G/A
6-2: 24476211A/T
6-2: 24479660C/T
6-2: 24479678T/C
6-2: 24479685G/A
6-2: 24503703G/T
6-2: 24503716C/T
6-2: 24503779T/G
6-2: 24672588C/T
6-2: 24771513T/C
6-2: 24789278G/A
6-2: 24801679G/A
6-2: 24884340C/T
6-2: 24941206A/G
6-2: 24981608C/T
6-2: 25069903A/C
6-2: 25139257A/C
6-2: 25185711T/C
6-2: 25210839C/T
6-2: 25246056A/G
6-2: 25276363A/C
6-2: 25306258T/C
6-2: 25357328A/T
6-2: 25493756A/C
6-2: 25524727G/A
6-2: 25553632G/A
6-2: 25574545T/G
6-2: 25633452C/A
6-2: 25748060C/T
6-2: 25758115G/A
6-2: 25794670G/C
6-2: 25814368G/T
6-2: 25878352A/G
6-2: 25893340A/G
6-2: 25904707T/G
6-2: 25928918G/C
6-2: 25938842A/.
6-2: 25952460C/T
6-2: 25963294A/G
6-2: 26031468A/G
6-2: 26055114T/C
6-2: 26104975A/T
6-2: 26130398G/A
6-2: 26159487C/T
6-2: 26201507C/G
6-2: 26232242A/T
6-2: 26268787T/C
6-2: 26336984A/G
6-2: 26582499G/A
6-2: 26600482C/T
6-2: 26649636A/G
6-2: 26664469A/C
6-2: 26676604T/A
6-2: 26683610T/A
6-2: 26687708T/C
6-2: 26700205T/C
6-2: 26711589G/T
6-2: 26725144G/A
6-2: 26739948A/C
6-2: 26740087T/C
6-2: 26740481A/G
6-2: 26768692A/T
6-2: 26784174A/G
6-2: 26799319G/A
6-2: 26811789A/T
6-2: 26813925C/T
6-2: 26814012T/C
6-2: 26864247C/G
6-2: 26948583C/A
6-2: 27044629T/G
6-2: 27057039G/A
6-2: 27069216G/A
6-2: 27069787T/C
6-2: 27070776C/A
6-2: 27115424A/G
6-2: 27158885T/A
6-2: 27186459T/A
6-2: 27199214A/T
6-2: 27241261T/C
6-2: 27328899G/A
6-2: 27362676A/C
6-2: 27380529A/G
6-2: 27455952T/A
6-2: 27482243T/A
6-2: 27510493C/A
6-2: 27703502C/T
6-2: 27720766T/C
6-2: 27730771C/A
6-2: 27766147G/A
6-2: 27798239C/T
6-2: 28321873A/C
6-2: 28322936A/G
6-2: 28334521G/A
6-2: 28345589A/G
6-2: 28358417T/G
6-2: 28389558G/A
6-2: 28408533G/A
6-2: 28469732G/A
6-2: 28488852A/T
6-2: 28502735A/C
6-2: 28592113A/G
6-2: 28665723T/G
6-2: 28682728A/G
6-2: 28719178G/A
6-2: 28723135C/T
6-2: 28811024C/T
6-2: 28869347C/T
6-2: 28909940C/A
6-2: 28958235G/A
6-2: 28991059A/C
6-2: 29018153T/C
6-2: 29138860A/G
6-2: 29159034C/T
6-2: 29163521G/A
6-2: 29182583A/T
6-2: 29226844A/G
6-2: 29272248C/G
6-2: 29349058G/A
6-2: 29360110A/T
6-2: 29398697A/C
6-2: 29476926G/A
6-2: 29493206T/C
6-2: 29515018T/C
6-2: 29532233C/T
6-2: 29535750T/C
6-2: 29542937G/A
6-2: 29613945G/T
6-2: 29683265C/T
6-2: 29698925T/G
6-2: 29711511T/G
6-2: 29802829A/G
6-2: 29872625C/A
6-2: 29894227G/A
6-2: 29980495C/T
6-2: 30184470G/T
6-2: 30195148T/A
6-2: 30283778T/C
6-2: 30303248C/T
6-2: 30326500A/G
6-2: 30339597G/A
6-2: 30370743G/T
6-2: 30414697C/T
6-2: 30430926C/G
6-2: 30453562C/T
6-2: 30487938A/T
6-2: 30608604C/G
6-2: 30646655A/G
6-2: 30670475A/T
6-2: 30759679T/C
6-2: 30831772A/G
6-2: 30900650C/T
6-2: 31002982T/G
6-2: 31036408T/C
6-2: 31107714G/A
6-2: 31166877T/C
6-2: 31225492A/T
6-2: 31251855T/G
6-2: 31309510C/T
6-2: 31309932A/G
6-2: 31326079T/G
6-2: 31353499C/A
6-2: 31371850G/A
6-2: 31408125T/A
6-2: 31443351G/T
6-2: 31475911C/A
6-2: 31513974C/T
6-2: 31539681A/T
6-2: 31627896G/A
6-2: 31738751T/A
6-2: 31767348A/C
6-2: 31811434A/G
6-2: 31850965G/A
6-2: 31877445A/C
6-2: 31906762G/C
6-2: 31911913G/C
6-2: 31913216G/A
6-2: 31914434T/A
6-2: 31914440A/T
6-2: 31917941C/T
6-2: 31918160C/T
6-2: 31918844G/T
6-2: 31920260C/T
6-2: 31931098A/G
6-2: 31947415G/A
6-2: 31968138C/T
6-2: 31997754A/T
6-2: 32055955A/G
6-2: 32123960G/A
6-2: 32254047T/C
6-2: 32281956T/C
6-2: 32310233C/T
6-2: 32333532C/T
6-2: 32403341G/A
6-2: 32534432A/G
6-2: 32847122A/T
6-2: 32946124C/T
6-2: 33029529T/C
6-2: 33045650T/C
6-2: 33355960A/C
6-2: 33433330A/T
6-2: 33433445T/C
6-2: 33627330T/G
6-2: 33819689T/C
6-2: 33830710C/T
6-2: 33919765G/A
6-2: 33973432T/A
6-2: 34085331G/C
6-2: 34092992C/T
6-2: 34118020A/C
6-2: 34173873C/T
6-2: 34375489T/C
6-2: 34394898G/T
6-2: 34395365C/T
6-2: 34396036A/G
6-2: 34439359C/T
6-2: 34466454G/A
6-2: 34502102A/T
6-2: 34544285C/T
6-2: 34672550C/G
6-2: 34677952G/A
6-2: 34780924C/A
6-2: 34782998C/G
6-2: 34895063T/G
6-2: 34896732G/C
6-2: 34913834A/G
6-2: 35188292G/A
6-2: 35433829C/G
6-2: 35610658A/G
6-2: 35613335C/T
6-2: 35650841G/T
6-2: 35696817G/A
6-2: 35753468C/T
6-2: 35826189T/C
6-2: 35883206C/T
6-2: 35936336T/C
6-2: 36431458T/G
6-2: 36435251T/C
6-2: 36688470T/C
6-2: 37070303C/A
6-2: 37400593T/G
6-2: 37500206A/G
6-2: 37548799G/T
6-2: 37694828C/T
6-2: 37815833T/C
6-2: 37826280A/T
6-2: 37857171C/T
6-2: 37877349T/C
6-2: 38124149A/T
6-2: 38124160T/C
6-2: 38124213A/T
6-2: 38148273T/C
6-2: 38186860A/G
6-2: 38233665C/A
6-2: 38297924C/T
6-2: 38401807C/T
6-2: 38417201C/T
6-2: 38423530T/C
6-2: 38432457G/T
6-2: 38446417C/T
6-2: 38473120T/A
6-2: 38487299G/T
6-2: 38503523G/T
6-2: 38503654T/C
6-2: 38560344C/T
6-2: 38591007C/T
6-2: 38632797T/C
6-2: 38740289A/T
6-2: 38741336T/A
6-2: 38756223T/C
6-2: 38823522G/A
6-2: 38823929T/C
6-2: 38855563G/T
6-2: 38952660C/T
6-2: 38992265A/T
6-2: 39108243C/A
6-2: 39266063G/A
6-2: 39393453T/A
6-2: 39456429C/T
6-2: 39519416G/A
6-2: 39576669T/A
6-2: 39608036C/T
6-2: 39622314T/C
6-2: 39721343C/A
6-2: 39923342G/T
6-2: 39955660G/T
6-2: 40171587G/T
6-2: 40181668G/A
6-2: 40232560T/G
6-2: 40341942G/T
6-2: 40416327G/C
6-2: 40591543G/A
6-2: 40657228C/A
6-2: 40778156C/T
6-2: 40782367C/A
6-2: 40850196A/G
6-2: 40992159C/A
6-2: 41116227T/C
6-2: 41272951C/T
6-2: 41306783T/A
6-2: 41418609T/G
6-2: 41421632A/G
6-2: 41500149A/C
6-2: 41604401A/G
6-2: 41693111G/A
6-2: 41744095C/T
6-2: 41806255G/A
6-2: 41816576G/A
6-2: 42143471C/G
6-2: 42180083T/G
6-2: 42274034C/A
6-2: 42594860C/G
6-2: 42860779C/T
6-2: 42903629C/T
6-2: 43109664T/C
6-2: 43152306T/C
6-2: 43450948A/T
6-2: 43504349T/C
6-2: 43616087A/G
6-2: 43643783A/G
6-2: 43722190G/T
6-2: 43848850T/C
6-2: 43849195A/G
6-2: 43944200A/C
6-2: 44108043A/G
6-2: 44145307G/A
6-2: 44207038C/G
6-2: 44220748T/C
6-2: 44226298A/G
6-2: 44250082G/C
6-2: 44375120A/G
6-2: 44411916C/G
6-2: 44481127C/A
6-2: 44522522T/A
6-2: 44553754T/G
6-2: 44686491G/A
6-2: 44860119A/G
6-2: 45083998G/A
6-2: 45111074C/T
6-2: 45189255A/T
6-2: 45235537C/A
6-2: 45271271C/T
6-2: 45323686T/C
6-2: 45380719G/A
6-2: 45419793C/T
6-2: 45421575A/G
6-2: 45434510T/C
6-2: 45457935C/T
6-2: 45473870T/A
6-2: 45508332T/C
6-2: 45536039T/C
6-2: 45684792T/A
6-2: 45739641T/A
6-2: 45776852A/G
6-2: 45819178A/C
6-2: 45859633A/C
6-2: 46013063A/C
6-2: 46094658C/T
6-2: 46102664T/A
6-2: 46127320C/T
6-2: 46127550T/A
6-2: 46129083C/T
6-2: 46148514A/G
6-2: 46149803C/T
6-2: 46149833T/C
6-2: 46149911C/T
6-2: 46150007G/A
6-2: 46150202G/A
6-2: 46150446C/T
6-2: 46163079G/T
6-2: 46247278A/T
6-2: 46385049G/A
6-2: 46458894G/A
6-2: 46647776G/C
6-2: 46660501T/A
6-2: 46783014C/T
6-2: 46822636A/T
6-2: 46850419A/C
6-2: 46983504A/G
6-2: 46994959A/T
6-2: 47002973C/T
6-2: 47003594C/T
6-2: 47034582A/T
6-2: 47063059T/C
6-2: 47124834C/G
6-2: 47209784A/T
6-2: 47420793C/T
6-2: 47459262A/G
6-2: 47490897A/G
6-2: 47565391T/G
6-2: 47723558G/A
6-2: 47724930G/T
6-2: 47759889T/C
6-2: 47761659A/G
6-2: 47852206T/C
6-2: 47852288G/T
6-2: 47956547T/A
6-2: 48043734T/C
6-2: 48132185C/T
6-2: 48209005C/T
6-2: 48475686G/A
6-2: 48514938T/C
6-2: 48582446A/C
6-2: 48597000T/G
6-2: 48631903C/T
6-2: 48724588A/G
6-2: 48770801A/G
6-2: 48833780A/T
6-2: 48851605A/T
6-2: 48894722C/T
6-2: 48926270C/T
6-2: 48974245A/G
6-2: 49004962G/T
6-2: 49057949G/T
6-2: 49294568G/A
6-2: 49349318C/G
6-2: 49462792G/A
6-2: 49743000C/T
6-2: 49851763G/A
6-2: 50051000T/C
6-2: 50096336G/A
6-2: 50290895T/C
6-2: 50291151G/A
6-2: 50293932T/A
6-2: 50302734A/G
6-2: 50313647G/T
6-2: 50338310G/A
6-2: 50357776G/A
6-2: 50412094A/T
6-2: 50415584G/A
6-2: 50416152C/T
6-2: 50416759T/G
6-2: 50417779T/A
6-2: 50453433T/C
6-2: 50483774C/A
6-2: 50524976T/C
6-2: 50548657C/T
6-2: 50559642C/T
6-2: 50588310C/T
6-2: 50627914A/G
6-2: 50700440G/T
6-2: 50707162T/A
6-2: 50711033G/T
6-2: 50734800G/A
6-2: 50760039C/G
6-2: 51020955A/C
6-2: 51047463G/C
6-2: 51099495C/T
6-2: 51196795C/T
6-2: 51281652T/C
6-2: 51302660A/T
6-2: 51427504C/A
6-2: 51472460A/T
6-2: 51505772G/A
6-2: 51518669T/A
6-2: 51587872T/A
6-2: 51645479G/A
6-2: 51659137G/A
6-2: 51659287A/G
6-2: 51659427T/C
6-2: 51659895T/A
6-2: 51660043G/T
6-2: 51660160A/T
6-2: 51683761C/T
6-2: 51687735A/T
6-2: 51694521C/A
6-2: 51742068A/C
6-2: 51780980G/T
6-2: 51817204C/A
6-2: 51817222C/A
6-2: 51818754A/G
6-2: 51819142T/C
6-2: 51977393C/T
6-2: 52313224G/A
6-2: 52346415C/A
6-2: 52415613C/T
6-2: 52440891C/T
6-2: 52569125T/G
6-2: 52671391G/A
6-2: 52699827G/A
6-2: 52710943G/A
6-2: 52738338T/C
6-2: 52749181G/A
6-2: 52833347T/A
6-2: 52858680T/C
6-2: 52872659G/A
6-2: 52900700T/A
6-2: 52962916G/A
6-2: 52974094A/G
6-2: 52976127A/G
6-2: 53112219C/T
6-2: 53218841G/A
6-2: 53259778G/A
6-2: 53289329G/A
6-2: 53312277T/C
6-2: 53354903C/T
6-2: 53376259T/C
6-2: 53455297G/T
6-2: 53468909G/A
6-2: 53492954C/G
6-2: 53520292G/A
6-2: 53599243T/C
6-2: 53611467T/C
6-2: 53692347C/T
6-2: 53725031T/A
6-2: 53738836G/A
6-2: 53777115A/C
6-2: 53824074G/A
6-2: 53837928G/A
6-2: 53863412A/T
6-2: 53873533G/T
6-2: 53885257C/T
6-2: 53900161G/A
6-2: 53915154T/A
6-2: 53925754A/G
6-2: 54003823T/A
6-2: 54026217T/G
6-2: 54048457A/T
6-2: 54069403G/A
6-2: 54081948A/G
6-2: 54086276A/T
6-2: 54093095T/G
6-2: 54106664A/G
6-2: 54232154C/T
6-2: 54294673T/G
6-2: 54382801C/T
6-2: 54426167T/A
6-2: 54459380G/C
6-2: 54545801T/A
6-2: 54938178A/G
6-2: 54939726C/T
6-2: 54953556G/T
6-2: 54973998A/T
6-2: 55026274A/C
6-2: 55068519C/T
6-2: 55323907T/C
6-2: 55340475C/A
6-2: 55350908T/C
6-2: 55354002T/C
6-2: 55386924T/G
6-2: 55438275T/C
6-2: 55484341G/A
6-2: 55530297C/T
6-2: 55581999T/A
6-2: 55628990C/T
6-2: 55699958G/A
6-2: 55762982T/C
6-2: 55783961G/T
6-2: 55818679G/T
6-2: 55831497G/T
6-2: 55874999A/C
6-2: 55908945A/G
6-2: 55919524T/C
6-2: 55950776C/A
6-2: 55977800T/C
6-2: 55991740A/G
6-2: 55991755T/C
6-2: 56142778G/A
6-2: 56273875C/A
6-2: 56325156T/C
6-2: 56468376T/G
6-2: 56589129T/C
6-2: 56599848T/C
6-2: 56624907C/T
6-2: 56717303T/A
6-2: 56761387G/C
6-2: 56795590C/A
6-2: 56901311T/C
6-2: 56926263T/C
6-2: 56976162G/T
6-2: 57024118C/T
6-2: 57038157G/A
6-2: 57046345A/C
6-2: 57073017A/G
6-2: 57073021G/C
6-2: 57096121A/G
6-2: 57096512T/G
6-2: 57097439C/T
6-2: 57181069G/A
6-2: 57182820A/G
6-2: 57209364C/T
6-2: 57256139G/A
6-2: 57267247A/G
6-2: 57374590C/T
6-2: 57400914A/C
6-2: 57451153C/T
6-2: 57532774G/A
6-2: 57609941G/A
6-2: 57657513G/T
6-2: 57671799T/C
6-2: 57696353C/T
6-2: 57718643A/G
6-2: 57727773G/A
6-2: 57754876T/A
6-2: 57787765C/T
6-2: 57861773T/G
6-2: 57948208G/A
6-2: 57959967T/A
6-2: 57984232C/T
6-2: 58002330C/T
6-2: 58083490A/G
6-2: 58099583G/T
6-2: 58127990A/T
6-2: 58221511A/G
6-2: 58247069A/T
6-2: 58332076G/A
6-2: 58466099A/G
6-2: 58491928C/T
6-2: 58497249T/G
6-2: 58497395G/A
6-2: 58497743G/A
6-2: 58498828G/T
6-2: 58499081G/A
6-2: 58499211C/T
6-2: 58499313C/T
6-2: 58499337T/C
6-2: 58499464C/T
6-2: 58499511A/G
6-2: 58542839G/A
6-2: 58581330T/A
6-2: 58608083T/G
6-2: 58620716C/A
6-2: 58629986T/C
6-2: 58631444T/A
6-2: 58648323T/G
6-2: 58676935C/T
6-2: 58678201C/T
6-2: 58678716T/A
6-2: 58691953G/A
6-2: 58695688T/C
6-2: 58705332C/T
6-2: 58719076T/C
6-2: 58748895A/G
6-2: 58750147G/A
6-2: 58765912G/A
6-2: 58802909A/T
6-2: 58877980G/A
6-2: 58967683A/T
6-2: 59010718T/G
6-2: 59183817A/G
6-2: 59273940A/C
6-2: 59331923A/G
6-2: 59366914T/C
6-2: 59368871G/C
6-2: 59368980T/C
6-2: 59369036C/A
6-2: 59369043T/A
6-2: 59385318A/C
6-2: 59391098C/T
6-2: 59403921G/A
6-2: 59487962C/T
6-2: 59535304G/A
6-2: 59563922A/G
6-2: 59647197T/A
6-2: 59674951A/G
6-2: 59705587C/G
6-2: 59742237A/T
6-2: 59794230A/G
6-2: 59835909T/A
6-2: 59892952A/T
6-2: 59922216A/T
6-2: 59950443T/G
6-2: 59963404T/C
6-2: 59973946C/T
6-2: 60064173C/T
6-2: 60082675A/T
6-2: 60098274A/T
6-2: 60117914C/T
6-2: 60190519T/C
6-2: 60292394C/T
6-2: 60337454T/C
6-2: 60400762T/C
6-2: 60427598T/C
6-2: 60451700C/T
6-2: 60451827A/T
6-2: 60466838C/T
6-2: 60494748A/C
6-2: 60556533A/G
6-2: 60567904C/A
6-2: 60580810A/G
6-2: 60618311G/C
6-2: 60645096T/C
6-2: 60691160T/C
6-2: 60723357T/A
6-2: 60723471A/T
6-2: 60723635C/A
6-2: 60751645A/C
6-2: 60764506G/A
6-2: 60793667C/A
6-2: 60804136A/T
6-2: 60814286A/G
6-2: 60857249C/T
6-2: 60890969T/A
6-2: 60906544G/A
6-2: 60917450A/G
6-2: 60918398T/G
6-2: 60978640G/A
6-2: 60994248G/A
6-2: 61065088T/A
6-2: 61106415C/A
6-2: 61155328A/G
6-2: 61251138C/A
6-2: 61259258T/C
6-2: 61303714C/T
6-2: 61349634C/T
6-2: 61362455C/T
6-2: 61405699A/G
6-2: 61434426C/A
6-2: 61470611T/C
6-2: 61486957G/A
6-2: 61530830T/G
6-2: 61620269A/G
6-2: 61658166A/T
6-2: 61663748A/G
6-2: 61664122A/G
6-2: 61732641A/T
6-2: 61745588G/A
6-2: 61784106T/A
6-2: 61849101A/T
6-2: 61875568C/T
6-2: 61915352C/T
6-2: 61961119G/A
6-2: 62027367T/C
6-2: 62038100G/A
6-2: 62081342C/A
6-2: 62153851T/C
6-2: 62197636T/C
6-2: 62197639A/G
6-2: 62197707T/G
6-2: 62197746T/G
6-2: 62199876C/A
6-2: 62232494C/A
6-2: 62237650C/T
6-2: 62262891A/G
6-2: 62308165T/C
6-2: 62322179G/A
6-2: 62440278A/T
6-2: 62504309A/G
6-2: 62577588C/T
6-2: 62589168C/T
6-2: 62709930A/T
6-2: 62714954G/A
6-2: 62744947A/T
6-2: 62766767G/T
6-2: 62770497T/A
6-2: 62797203A/G
6-2: 62856025G/A
6-2: 62866321T/C
6-2: 62876624G/T
6-2: 62879163A/T
6-2: 62893311G/T
6-2: 62920831C/G
6-2: 62976896A/T
6-2: 63037947C/T
6-2: 63097896T/C
6-2: 63137755C/T
6-2: 63178814A/G
6-2: 63257764G/T
6-2: 63308240C/T
6-2: 63344954T/C
6-2: 63357893A/G
6-2: 63369704G/A
6-2: 63416035G/T
6-2: 63438359T/C
6-2: 63497387A/G
6-2: 63530597C/T
6-2: 63804559T/A
6-2: 63807169C/A
6-2: 63824422T/C
6-2: 63843363A/G
6-2: 63935432C/A
6-2: 63983429A/T
6-2: 64011498G/A
6-2: 64019052C/G
6-2: 64019308T/A
6-2: 64019765G/A
6-2: 64019848A/G
6-2: 64085058C/T
6-2: 64106577A/C
6-2: 64115176G/A
6-2: 64162447T/C
6-2: 64177147T/C
6-2: 64196907T/A
6-2: 64222676T/G
6-2: 64304821C/T
6-2: 64402840A/C
6-2: 64427266G/A
6-2: 64451500G/A
6-2: 64489455C/T
6-2: 64500877A/T
6-2: 64536243C/T
6-2: 64546334C/T
6-2: 64571962C/T
6-2: 64724290A/G
6-2: 64724512G/T
6-2: 64811318G/T
6-2: 64833853C/T
6-2: 64863256T/C
6-2: 64949494G/T
6-2: 65013857A/G
6-2: 65051164G/A
6-2: 65139627T/C
6-2: 65158828T/C
6-2: 65267860C/T
6-2: 65334314G/A
6-2: 65338314G/A
6-2: 65389735C/G
6-2: 65415108A/G
6-2: 65434345T/A
6-2: 65476885T/C
6-2: 65534137G/A
6-2: 65562367A/G
6-2: 65568398A/C
6-2: 65598017G/A
6-2: 65633701T/C
6-2: 65663625A/G
6-2: 65783502G/T
6-2: 65801437T/A
6-2: 65846446T/G
6-2: 65911177G/C
6-2: 65986392C/A
6-2: 66030538C/A
6-2: 66097642T/A
6-2: 66116275C/G
6-2: 66134318T/A
6-2: 66144525T/C
6-2: 66157758G/A
6-2: 66169598A/G
6-2: 66181656G/C
6-2: 66192175C/T
6-2: 66203246A/G
6-2: 66205031T/C
6-2: 66211297G/C
6-2: 66213419G/C
6-2: 66243625A/G
6-2: 66323297T/C
6-2: 66354845T/G
6-2: 66395398A/G
6-2: 66442241T/C
6-2: 66476274G/A
6-2: 66512757T/C
6-2: 66713683C/T
6-2: 66717463C/T
6-2: 66731634T/C
6-2: 66780685A/G
6-2: 66868021A/T
6-2: 66881050A/C
6-2: 66901472G/C
6-2: 66911515A/C
6-2: 66912775A/C
6-2: 66976348G/A
6-2: 67002394G/A
6-2: 67029058T/C
6-2: 67039948A/T
6-2: 67091307G/A
6-2: 67135252G/A
6-2: 67135327A/T
6-2: 67135399C/T
6-2: 67135529G/A
6-2: 67197882G/A
6-2: 67229415T/C
6-2: 67229688A/G
6-2: 67248182T/G
6-2: 67258422A/T
6-2: 67328113A/G
6-2: 67354910C/T
6-2: 67416657T/A
6-2: 67418757A/G
6-2: 67419015G/A
6-2: 67419367G/A
6-2: 67419430G/A
6-2: 67419452A/G
6-2: 67587226A/T
6-2: 67603319C/A
6-2: 67604112G/T
6-2: 67604169G/A
6-2: 67604190T/G
6-2: 67604210A/G
6-2: 67774392A/G
6-2: 67784287T/C
6-2: 67813015T/C
6-2: 67872352G/A
6-2: 67913864C/T
6-2: 67913904T/G
6-2: 67915183G/A
6-2: 67994518T/A
6-2: 68009333T/C
6-2: 68092112C/T
6-2: 68168491G/A
6-2: 68404090A/G
6-2: 68415198A/G
6-2: 68482843G/A
6-2: 68534177A/G
6-2: 68583713G/A
6-2: 68638260A/T
6-2: 68664603C/T
6-2: 68739794A/T
6-2: 68797340C/T
6-2: 68840455C/T
6-2: 68860546G/A
6-2: 68891321G/T
6-2: 68891550G/T
6-2: 68893252T/C
6-2: 68893358T/C
6-2: 68910225C/T
6-2: 68921361T/C
6-2: 69018686G/T
6-2: 69031964C/T
6-2: 69072869A/G
6-2: 69117221C/T
6-2: 69145300A/G
6-2: 69176668T/A
6-2: 69207721A/G
6-2: 69223661C/T
6-2: 69224331T/A
6-2: 69239293C/T
6-2: 69297144G/A
6-2: 69345854G/T
6-2: 69380940T/C
6-2: 69384815C/T
6-2: 69392523G/T
6-2: 69414877T/C
6-2: 69456942C/T
6-2: 69551948A/C
6-2: 69638565A/G
6-2: 69664357A/C
6-2: 69666571T/C
6-2: 69670359G/T
6-2: 69670515C/T
6-2: 69674670G/T
6-2: 69692301T/C
6-2: 69699269T/A
6-2: 69702877A/G
6-2: 69713284T/C
6-2: 69723733T/C
6-2: 69723930T/C
6-2: 69724200A/G
6-2: 69724228A/G
6-2: 69724646G/A
6-2: 69725296A/G
6-2: 69725414C/T
6-2: 69725573G/A
6-2: 69738129C/A
6-2: 69738737T/A
6-2: 69749612G/C
6-2: 69811184G/A
6-2: 69905847T/C
6-2: 69917026A/G
6-2: 69930534T/C
6-2: 69945245G/A
6-2: 69990085T/A
6-2: 69990282T/A
6-2: 70051900A/G
6-2: 70139673T/C
6-2: 70269535C/G
6-2: 70273163C/G
6-2: 70283848T/C
6-2: 70312665C/A
6-2: 70364917C/T
6-2: 70398090A/G
6-2: 70412132T/G
6-2: 70492408T/G
6-2: 70580259T/G
6-2: 70658897T/C
6-2: 70670014C/T
6-2: 70705009A/G
6-2: 70749696C/G
6-2: 70769617C/T
6-2: 70781519A/G
6-2: 70803554G/A
6-2: 70849362C/G
6-2: 70879834A/T
6-2: 70894738G/A
6-2: 70963340T/C
6-2: 70973155T/C
6-2: 70980198A/T
6-2: 71021737A/T
6-2: 71086055C/T
6-2: 71145117T/C
6-2: 71244982A/T
6-2: 71277839C/T
6-2: 71303637T/C
6-2: 71303942A/G
6-2: 71304116G/A
6-2: 71304611G/A
6-2: 71304718T/C
6-2: 71333377G/C
6-2: 71413089G/T
6-2: 71477662G/A
6-2: 71509509G/A
6-2: 71544246T/A
6-2: 71547956G/A
6-2: 71631280A/G
6-2: 71731359A/C
6-2: 71741947G/A
6-2: 71795415T/C
6-2: 71826166C/T
6-2: 71923619A/G
6-2: 71950058T/A
6-2: 71975684C/T
6-2: 72006716T/G
6-2: 72036914T/G
6-2: 72065611G/T
6-2: 72105063C/T
6-2: 72161600A/G
6-2: 72341101A/T
6-2: 72380972C/T
6-2: 72417467T/A
6-2: 72450393C/G
6-2: 72495171G/A
6-2: 72558144A/T
6-2: 72580109A/G
6-2: 72590806G/A
6-2: 72619097A/G
6-2: 72683818A/G
6-2: 72714492C/T
6-2: 72747262T/A
6-2: 72820550G/A
6-2: 72915973G/A
6-2: 72961393A/G
6-2: 72987723T/C
6-2: 73114567A/G
6-2: 73128138G/A
6-2: 73148649T/G
6-2: 73190049G/A
6-2: 73200758G/C
6-2: 73200891A/G
6-2: 73200914G/C
6-2: 73250354A/T
6-2: 73294043T/C
6-2: 73306212T/A
6-2: 73319634C/A
6-2: 73320015T/C
6-2: 73320074G/T
6-2: 73354457T/G
6-2: 73385481G/T
6-2: 73437979C/T
6-2: 73465655T/C
6-2: 73479906G/A
6-2: 73500992G/A
6-2: 73515700G/A
6-2: 73530370C/T
6-2: 73545506T/A
6-2: 73612276C/T
6-2: 73759068A/C
6-2: 73770787C/T
6-2: 73771076A/G
6-2: 73783567A/G
6-2: 73796680T/A
6-2: 73814706C/A
6-2: 73914732A/G
6-2: 73924937C/T
6-2: 73968787A/G
6-2: 74038590T/C
6-2: 74207306T/C
6-2: 74222027G/A
6-2: 74242076A/G
6-2: 74252334T/C
6-2: 74262759T/A
6-2: 74322449G/A
6-2: 74620904C/A
6-2: 74807978G/C
6-2: 74985419C/T
6-2: 75056586C/T
6-2: 75067677A/T
6-2: 75104742C/A
6-2: 75122917G/A
6-2: 75129257G/T
6-2: 75175380G/T
6-2: 75233460G/A
6-2: 75291514T/C
6-2: 75325260G/A
6-2: 75509849C/T
6-2: 75625394G/T
6-2: 75714756A/G
6-2: 75758118A/G
6-2: 75854450T/A
6-2: 75977305G/A
6-2: 76117447G/A
6-2: 76118653C/T
6-2: 76188410G/A
6-2: 76300167C/G
6-2: 76589906G/A
6-2: 76606267A/C
6-2: 76671895C/A
6-2: 76774929A/T
6-2: 76776270G/T
6-2: 76806756G/A
6-2: 76815446A/C
6-2: 76819229A/C
6-2: 76894381C/T
6-2: 76909717T/C
6-2: 76997346C/T
6-2: 77012342T/C
6-2: 77013978T/C
6-2: 77022823T/C
6-2: 77030805C/T
6-2: 77031121T/A
6-2: 77057158T/G
6-2: 77084551T/A
6-2: 77263987C/T
6-2: 77358011G/A
6-2: 77510929G/A
6-2: 77551481C/T
6-2: 77567362C/T
6-2: 77618003C/T
6-2: 77675837C/T
6-2: 77826102C/T
6-2: 77827566G/A
6-2: 77857150T/G
6-2: 77939611G/T
6-2: 78428907C/T
6-2: 78457999T/G
6-2: 78492651T/A
6-2: 78507499G/A
6-2: 78525761C/T
6-2: 78544843G/A
6-2: 78570313T/A
6-2: 78588913G/A
6-2: 78632850A/G
6-2: 78665353A/T
6-2: 78693569C/A
6-2: 78732688G/C
6-2: 78762970C/A
6-2: 78802716C/T
6-2: 78827467T/C
6-2: 78837563G/A
6-2: 78874932G/A
6-2: 78917821A/C
6-2: 78966014T/A
6-2: 79021372G/A
6-2: 79059195T/C
6-2: 79094115A/T
6-2: 79110526T/A
6-2: 79127788G/A
6-2: 79177079T/C
6-2: 79208968G/A
6-2: 79210814C/T
6-2: 79276030G/T
6-2: 79361384C/G
6-2: 79386625T/A
6-2: 79420549C/T
6-2: 79449580A/G
6-2: 79484937G/A
6-2: 79514590T/C
6-2: 79550879C/T
6-2: 79590200G/A
6-2: 79626548G/A
6-2: 79658551C/T
6-2: 79747538T/G
6-2: 79775287C/T
6-2: 79811926A/T
6-2: 79847499G/A
6-2: 79883466A/T
6-2: 79926838T/C
6-2: 79976329T/A
6-2: 80006378A/G
6-2: 80035421G/A
6-2: 80056707T/C
6-2: 80071743A/T
6-2: 80092340T/C
6-2: 80112020C/T
6-2: 80147677T/C
6-2: 80207009A/T
6-2: 80457149C/T
6-2: 80747806C/T
6-2: 80776093G/A
6-2: 80813526A/C
6-2: 80878246A/G
6-2: 81039289C/A
6-2: 81089719C/T
6-2: 81119445T/G
6-2: 81149962G/T
6-2: 81167503C/T
6-2: 81266481T/G
6-2: 81324582A/G
6-2: 81736753C/T
6-2: 81757957A/G
6-2: 82888387G/A
6-2: 83259811T/C
6-3: 43067A/T
6-3: 82345A/T
6-3: 124603A/G
6-3: 145573G/A
6-3: 243222G/A
6-3: 302319A/G
6-3: 453105G/A
6-3: 530481A/G
6-3: 556682G/A
6-3: 615308G/A
6-3: 675419G/A
6-3: 732831A/T
6-3: 764072G/A
6-3: 790848A/G
6-3: 822775C/A
6-3: 922011T/C
6-3: 1037257T/A
6-3: 1068441C/G
6-3: 1104977A/T
6-3: 1125898G/A
6-3: 1302661C/T
6-3: 1356712C/T
6-3: 1390507C/G
6-3: 1421695A/G
6-3: 1504304T/G
6-3: 1578066G/A
6-3: 1628338C/T
6-3: 1628637A/G
6-3: 1645599A/G
6-3: 1649062G/C
6-3: 1664981T/A
6-3: 1706398C/T
6-3: 1780387T/G
6-3: 1794920G/T
6-3: 1918351C/A
6-3: 1976448T/C
6-3: 2020011G/T
6-3: 2102954T/G
6-3: 2111867C/T
6-3: 2114038T/C
6-3: 2229245G/C
6-3: 2268000T/C
6-3: 2269462T/A
6-3: 2280966A/G
6-3: 2289247C/G
6-3: 2303702G/C
6-3: 2410533T/A
6-3: 2425961A/G
6-3: 2507620G/A
6-3: 2529182G/A
6-3: 2561626A/G
6-3: 2571845A/G
6-3: 2572447G/A
6-3: 2582576G/T
6-3: 2633982G/A
6-3: 2648025C/T
6-3: 2669859A/G
6-3: 2698567G/A
6-3: 2764407A/T
6-3: 2829895A/G
6-3: 2859211T/C
6-3: 2880719T/C
6-3: 2981706G/A
6-3: 3137891A/G
6-3: 3173081G/A
6-3: 3183331T/C
6-3: 3228282T/A
6-3: 3267699T/A
6-3: 3280736T/C
6-3: 3383550T/C
6-3: 3397596A/G
6-3: 3410051T/C
6-3: 3488947T/A
6-3: 3637034G/A
6-3: 3695760T/C
6-3: 3722197T/A
6-3: 3765633A/G
6-3: 3784791T/A
6-3: 3799517C/A
6-3: 3809971G/A
6-3: 3834002C/G
6-3: 3903730T/A
6-3: 4001223T/A
6-3: 4001261C/T
6-3: 4001297G/A
6-3: 4001466C/T
6-3: 4011571C/T
6-3: 4165576T/C
6-3: 4165931A/T
6-3: 4166105A/G
6-3: 4166133A/G
6-3: 4166209G/A
6-3: 4174445C/T
6-3: 4308331C/A
6-3: 4414226G/A
6-3: 4515965T/C
6-3: 4525391A/G
6-3: 5027554G/A
6-3: 5033252C/A
6-3: 5034030T/C
6-3: 5034639T/A
6-3: 5037623C/T
6-3: 5051117A/G
6-3: 5064365T/A
6-3: 5176198G/C
6-3: 5183408T/C
6-3: 5203083C/T
6-3: 5214751A/G
6-3: 5264214G/A
6-3: 5276825G/A
6-3: 5301093G/T
6-3: 5465249A/G
6-3: 5467081A/G
6-3: 5481647A/G
6-3: 5491878C/T
6-3: 5492666G/A
6-3: 5492675G/A
6-3: 5495365G/A
6-3: 5503980A/G
6-3: 5518863T/A
6-3: 5541965A/C
6-3: 5630515T/A
6-3: 5655903T/C
6-3: 5684560T/C
6-3: 5687228T/C
6-3: 5687338G/A
6-3: 5689153T/C
6-3: 5721824A/G
6-3: 5766908A/C
6-3: 5881784C/A
6-3: 5925447T/A
6-3: 5967993T/C
6-3: 5985197A/G
6-3: 6050730T/C
6-3: 6092524G/A
6-3: 6128592T/C
6-3: 6151493G/A
6-3: 6193111G/T
6-3: 6203922C/T
6-3: 6204153G/T
6-3: 6214729T/A
6-3: 6243512T/C
6-3: 6243633G/A
6-3: 6245573G/A
6-3: 6245639A/T
6-3: 6245647G/T
6-3: 6253645C/T
6-3: 6274200C/T
6-3: 6296022T/C
6-3: 6323811C/T
6-3: 6352679G/A
6-3: 6366757C/T
6-3: 6376887C/T
6-3: 6377067G/A
6-3: 6391477T/C
6-3: 6391781G/A
6-3: 6404961C/T
6-3: 6447808C/T
6-3: 6463783C/T
6-3: 6484517T/C
6-3: 6484894A/G
6-3: 6528676C/T
6-3: 6607404G/T
6-3: 6649001T/A
6-3: 6700630T/G
6-3: 6805168A/G
6-3: 6817655A/G
6-3: 6848574G/C
6-3: 6850814G/T
6-3: 6860862A/G
6-3: 6935207T/A
6-3: 6986882T/C
6-3: 6998538C/T
6-3: 7012345C/T
6-3: 7018487C/T
6-3: 7033656T/A
6-3: 7085269T/C
6-3: 7106929T/G
6-3: 7112309T/C
6-3: 7145448G/A
6-3: 7163182C/T
6-3: 7165515A/G
6-3: 7167500G/A
6-3: 7184157T/C
6-3: 7205560C/T
6-3: 7255385C/A
6-3: 7290373G/T
6-3: 7368787T/C
6-3: 7421500A/G
6-3: 7449276G/A
6-3: 7474612T/G
6-3: 7509552A/G
6-3: 7591157G/A
6-3: 7591337C/T
6-3: 7596314G/A
6-3: 7613827T/C
6-3: 7665669C/T
6-3: 7838034G/A
6-3: 7887722G/A
6-3: 7915542A/T
6-3: 8003356T/C
6-3: 8031210A/G
6-3: 8064254C/T
6-3: 8201086C/T
6-3: 8253176G/A
6-3: 8255847C/T
6-3: 8257452A/G
6-3: 8280746C/A
6-3: 8294659A/C
6-3: 8358242C/T
6-3: 8359307G/A
6-3: 8392798A/T
6-3: 8445411G/A
6-3: 8505904A/G
6-3: 8551799C/A
6-3: 8595872A/C
6-3: 8607107A/G
6-3: 8635910T/C
6-3: 8667587C/T
6-3: 8696438G/T
6-3: 8721526T/G
6-3: 8751140A/G
6-3: 8815721C/T
6-3: 8830861T/A
6-3: 8884841T/C
6-3: 8921365C/T
6-3: 8991683C/G
6-3: 9014398G/A
6-3: 9163428A/G
6-3: 9350514A/G
6-3: 9361511G/A
6-3: 9392257G/A
6-3: 9419457C/T
6-3: 9465368G/T
6-3: 9465504T/A
6-3: 9495273A/T
6-3: 9510128G/A
6-3: 9535609C/A
6-3: 9557686G/T
6-3: 9567919C/T
6-3: 9583052T/C
6-3: 9600116T/C
6-3: 9622063C/T
6-3: 9633087A/G
6-3: 9633905G/T
6-3: 9634357G/A
6-3: 9635136C/A
6-3: 9635176C/T
6-3: 9637480G/A
6-3: 9643588G/A
6-3: 9653654T/G
6-3: 9744525A/G
6-3: 9785367G/T
6-3: 9828931T/C
6-3: 9857195T/C
6-3: 9914395C/A
6-3: 9947591C/T
6-3: 9965493C/G
6-3: 10091599A/G
6-3: 10118362A/G
6-3: 10160017A/C
6-3: 10160434T/C
6-3: 10160532C/T
6-3: 10160550G/A
6-3: 10167853T/A
6-3: 10169144T/A
6-3: 10182701T/G
6-3: 10249572A/G
6-3: 10249578G/A
6-3: 10271445G/A
6-3: 10376088A/G
6-3: 10429734C/T
6-3: 10485418C/A
6-3: 10522783C/T
6-3: 10532855A/C
6-3: 10598007C/G
6-3: 10635042G/A
6-3: 10688410A/T
6-3: 10688648T/C
6-3: 10691934A/C
6-3: 10720588T/C
6-3: 10800752T/C
6-3: 10845507C/T
6-3: 10892766C/T
6-3: 10936696A/T
6-3: 11036966A/T
6-3: 11079214A/T
6-3: 11128331G/A
6-3: 11130670A/G
6-3: 11192177C/T
6-3: 11210199G/A
6-3: 11238748G/T
6-3: 11295797C/T
6-3: 11379868A/T
6-3: 11401025C/T
6-3: 11505198G/T
6-3: 11679493A/T
6-3: 11696389T/G
6-3: 11715415G/A
6-3: 11715525C/T
6-3: 11715644A/C
6-3: 11715755T/C
6-3: 11718170T/A
6-3: 11718526A/G
6-3: 11718943G/T
6-3: 11740474C/T
6-3: 11805778T/C
6-3: 11825318A/T
6-3: 11859526G/T
6-3: 11911935C/T
6-3: 11912063T/C
6-3: 11931405T/C
6-3: 11931591T/A
6-3: 11949722A/T
6-3: 11978915A/T
6-3: 11988996T/C
6-3: 12001787G/A
6-3: 12022895C/T
6-3: 12054834C/T
6-3: 12084057T/C
6-3: 12095721G/T
6-3: 12131946G/A
6-3: 12194862C/A
6-3: 12205493T/C
6-3: 12235882G/A
6-3: 12250296A/G
6-3: 12255540A/T
6-3: 12255605G/T
6-3: 12286556A/G
6-3: 12293124A/T
6-3: 12330204A/T
6-3: 12381043A/C
6-3: 12416682T/A
6-3: 12463812A/G
6-3: 12476737G/A
6-3: 12513120A/G
6-3: 12524918A/T
6-3: 12536690G/A
6-3: 12539453A/T
6-3: 12597723G/A
6-3: 12608657G/A
6-3: 12611563T/C
6-3: 12659810C/A
6-3: 12684640G/A
6-3: 12721615C/T
6-3: 12762431T/C
6-3: 12816470A/G
6-3: 12835366A/G
6-3: 12895488A/T
6-3: 12934045G/T
6-3: 12989936A/T
6-3: 13019823T/C
6-3: 13034378T/A
6-3: 13047547C/T
6-3: 13063139G/A
6-3: 13168757T/C
6-3: 13196173T/C
6-3: 13202915G/A
6-3: 13223918T/C
6-3: 13248189A/T
6-3: 13270572A/G
6-3: 13281771G/C
6-3: 13298103G/A
6-3: 13310576T/C
6-3: 13325910C/T
6-3: 13336211C/T
6-3: 13369251C/T
6-3: 13380872G/C
6-3: 13486951A/G
6-3: 13624083A/T
6-3: 13625958A/G
6-3: 13630266T/A
6-3: 13630458G/A
6-3: 13630529G/A
6-3: 13631819T/C
6-3: 13632036G/A
6-3: 13632978A/T
6-3: 13639528T/C
6-3: 13671528C/T
6-3: 13702655C/T
6-3: 13732764A/C
6-3: 13774341G/A
6-3: 13801254A/G
6-3: 13837816G/A
6-3: 13854088A/G
6-3: 13867550G/C
6-3: 13878202C/T
6-3: 13897469A/C
6-3: 13925751T/A
6-3: 13926715G/A
6-3: 13944658G/A
6-3: 13983818A/G
6-3: 13996011C/T
6-3: 14010027T/C
6-3: 14013712G/C
6-3: 14013780C/T
6-3: 14021856C/T
6-3: 14022625G/A
6-3: 14032917A/T
6-3: 14033926G/A
6-3: 14034123G/A
6-3: 14034149G/T
6-3: 14035178C/T
6-3: 14111288A/G
6-3: 14167647C/A
6-3: 14201057G/A
6-3: 14238765A/C
6-3: 14273804T/C
6-3: 14341784T/G
6-3: 14357285T/C
6-3: 14509611G/T
6-3: 14509623A/T
6-3: 14509626C/A
6-3: 14509640A/G
6-3: 14509988A/G
6-3: 14522315G/A
6-3: 14545920A/G
6-3: 14546028C/T
6-3: 14546112T/C
6-3: 14547632G/A
6-3: 14552990A/T
6-3: 14553011T/G
6-3: 14556418A/G
6-3: 14564345T/A
6-3: 14566915C/G
6-3: 14585651C/T
6-3: 14599314T/G
6-3: 14645873T/G
6-3: 14655548T/C
6-3: 14655685C/T
6-3: 14655895T/C
6-3: 14660914C/T
6-3: 14666628G/A
6-3: 14666905T/G
6-3: 14681919T/C
6-3: 14730954C/A
6-3: 14848580T/G
6-3: 14935395G/A
6-3: 14950570G/A
6-3: 14950699G/T
6-3: 14952028C/T
6-3: 14984011G/A
6-3: 15095039C/T
6-3: 15147780A/T
6-3: 15227997A/C
6-3: 15230370A/G
6-3: 15244184T/C
6-3: 15244354C/T
6-3: 15256281C/T
6-3: 15274015T/C
6-3: 15278679G/A
6-3: 15278740G/A
6-3: 15278900G/A
6-3: 15278926T/C
6-3: 15279131C/G
6-3: 15279186C/T
6-3: 15279240T/C
6-3: 15291411C/G
6-3: 15297845T/C
6-3: 15298155T/C
6-3: 15298221C/A
6-3: 15298441A/G
6-3: 15298656A/G
6-3: 15302009T/A
6-3: 15303845C/T
6-3: 15303898C/T
6-3: 15304111C/A
6-3: 15309501C/T
6-3: 15312837G/A
6-3: 15314935A/T
6-3: 15367249C/G
6-3: 15376298T/C
6-3: 15378553A/G
6-3: 15382730T/A
6-3: 15383810C/G
6-3: 15384378A/T
6-3: 15384443C/T
6-3: 15385476G/C
6-3: 15400057A/T
6-3: 15426076G/A
6-3: 15450386G/A
6-3: 15476263C/T
6-3: 15551366A/G
6-3: 15585686T/C
6-3: 15613951A/G
6-3: 15674961G/A
6-3: 15692878A/G
6-3: 15696765C/T
6-3: 15707780T/C
6-3: 15730998A/G
6-3: 15767463A/T
6-3: 15771817G/A
6-3: 15808308G/A
6-3: 15824768G/A
6-3: 15847524C/A
6-3: 15886438G/A
6-3: 15920475G/A
6-3: 15984673C/G
6-3: 15996046T/C
6-3: 16007299C/T
6-3: 16038260T/C
6-3: 16095565G/T
6-3: 16126326C/T
6-3: 16161718G/A
6-3: 16172757T/A
6-3: 16191391T/C
6-3: 16287163C/T
6-3: 16320465A/G
6-3: 16336638T/C
6-3: 16356649G/A
6-3: 16416284C/A
6-3: 16465097G/T
6-3: 16487024A/G
6-3: 16498426C/T
6-3: 16502415A/C
6-3: 16502434C/A
6-3: 16502564G/A
6-3: 16506139G/T
6-3: 16516634C/G
6-3: 16530642A/T
6-3: 16547827G/A
6-3: 16558656G/A
6-3: 16560768T/C
6-3: 16563411G/A
6-3: 16661119T/C
6-3: 16717354A/G
6-3: 16767490A/G
6-3: 16797057T/C
6-3: 16801785G/T
6-3: 16808361G/T
6-3: 16867211C/T
6-3: 16892980T/C
6-3: 16904773C/T
6-3: 16921422C/T
6-3: 16931516C/A
6-3: 16931625G/T
6-3: 16931781C/T
6-3: 16942308T/C
6-3: 16951640T/A
6-3: 16951838G/T
6-3: 16974088C/T
6-3: 16997857A/G
6-3: 17101471A/C
6-3: 17105718A/G
6-3: 17133449T/G
6-3: 17174100C/T
6-3: 17197939A/T
6-3: 17326805C/T
6-3: 17335077C/T
6-3: 17353796C/T
6-3: 17429958C/A
6-3: 17466770G/C
6-3: 17500915G/T
6-3: 17505653A/G
6-3: 17623146A/T
6-3: 17624365C/T
6-3: 17624422A/G
6-3: 17624954A/G
6-3: 17625031C/A
6-3: 17636927T/C
6-3: 17640141A/G
6-3: 17647383C/A
6-3: 17651098A/T
6-3: 17654675C/T
6-3: 17665052G/A
6-3: 17675540T/G
6-3: 17688946G/A
6-3: 17752964C/G
6-3: 17753109G/A
6-3: 17753217A/G
6-3: 17777574G/A
6-3: 17788521C/G
6-3: 17788961G/A
6-3: 17789104G/A
6-3: 17789388G/A
6-3: 17790006C/A
6-3: 17795622C/T
6-3: 17795723A/T
6-3: 17826667C/A
6-3: 17861316A/G
6-3: 17891229A/G
6-3: 17947726C/T
6-3: 17979505C/G
6-3: 17981618G/A
6-3: 18007277G/T
6-3: 18007866G/C
6-3: 18008711A/G
6-3: 18008744G/C
6-3: 18008747A/T
6-3: 18008759A/T
6-3: 18039607C/T
6-3: 18039638C/T
6-3: 18039659C/A
6-3: 18039695C/T
6-3: 18039718C/T
6-3: 18039827C/T
6-3: 18039846G/A
6-3: 18039847C/T
6-3: 18039890C/G
6-3: 18039918G/T
6-3: 18039926A/T
6-3: 18040017C/A
6-3: 18040018C/G
6-3: 18040044T/A
6-3: 18040048A/G
6-3: 18040057T/G
6-3: 18040079A/T
6-3: 18040190T/.
6-3: 18040202G/A
6-3: 18040250A/C
6-3: 18040288G/T
6-3: 18040289A/T
6-3: 18040409A/G
6-3: 18040526G/T
6-3: 18040538A/G
6-3: 18040539A/G
6-3: 18077319G/C
6-3: 18091277T/C
6-3: 18102571A/G
6-3: 18102955T/C
6-3: 18103102T/C
6-3: 18107532G/T
6-3: 18112642T/C
6-3: 18122921C/T
6-3: 18125356G/A
6-3: 18151482C/A
6-3: 18152443C/T
6-3: 18152515C/T
6-3: 18152530A/T
6-3: 18152642C/T
6-3: 18156768C/T
6-3: 18156828G/A
6-3: 18312183C/T
6-3: 18319574T/A
6-3: 18330083A/T
6-3: 18334418T/C
6-3: 18336223T/C
6-3: 18336338G/A
6-3: 18336971A/C
6-3: 18336975C/G
6-3: 18337160G/A
6-3: 18337344T/G
6-3: 18337589T/C
6-3: 18337777A/C
6-3: 18339229C/T
6-3: 18345184A/T
6-3: 18395730C/T
6-3: 18431175G/A
6-3: 18524262C/A
6-3: 18547728C/T
6-3: 18550480C/T
6-3: 18564967A/G
6-3: 18575587G/A
6-3: 18604101C/T
6-3: 18617021G/T
6-3: 18627216T/G
6-3: 18667932C/T
6-3: 18795815G/A
6-3: 18797751C/T
6-3: 18822669T/C
6-3: 18898453T/C
6-3: 18947973G/A
6-3: 18983758T/C
6-3: 19001052C/A
6-3: 19002562A/G
6-3: 19035481T/C
6-3: 19065721T/C
6-3: 19073252C/T
6-3: 19073306C/T
6-3: 19106886G/A
6-3: 19125717G/C
6-3: 19156911C/T
6-3: 19158785T/A
6-3: 19175184A/G
6-3: 19186003T/C
6-3: 19218125C/T
6-3: 19249085T/C
6-3: 19256524G/T
6-3: 19256703C/T
6-3: 19275959G/A
6-3: 19279530C/G
6-3: 19295852A/T
6-3: 19348819G/T
6-3: 19365944C/T
6-3: 19386203G/A
6-3: 19390454T/A
6-3: 19402781T/C
6-3: 19425147G/A
6-3: 19442190C/G
6-3: 19448796C/G
6-3: 19449928C/T
6-3: 19450082C/T
6-3: 19455660G/A
6-3: 19457099G/A
6-3: 19457482T/C
6-3: 19457553T/C
6-3: 19539469A/T
6-3: 19559085T/A
6-3: 19593134G/A
6-3: 19640759T/C
6-3: 19658763G/A
6-3: 19781522G/A
6-3: 19787578T/A
6-3: 19808592T/C
6-3: 19854911C/A
6-3: 19876167T/A
6-3: 19877059A/G
6-3: 19878516T/A
6-3: 19882955T/A
6-3: 19888327A/T
6-3: 19896330T/C
6-3: 19904035A/G
6-3: 19924629C/T
6-3: 19936950G/A
6-3: 19956523G/A
6-3: 19975857C/T
6-3: 20006656C/T
6-3: 20049675T/C
6-3: 20066993G/A
6-3: 20107441A/T
6-3: 20122141G/A
6-3: 20148636G/A
6-3: 20183338G/C
6-3: 20193878A/G
6-3: 20209435T/C
6-3: 20223927C/T
6-3: 20225491G/A
6-3: 20254323G/T
6-3: 20346977C/T
6-3: 20358319G/T
6-3: 20369823G/T
6-3: 20369826C/A
6-3: 20369873C/T
6-3: 20369881A/G
6-3: 20369883T/C
6-3: 20369909T/C
6-3: 20369910A/T
6-3: 20369916A/T
6-3: 20369917C/T
6-3: 20369933A/C
6-3: 20370265G/T
6-3: 20370276G/.
6-3: 20370285T/C
6-3: 20370357T/C
6-3: 20370360T/C
6-3: 20370447A/G
6-3: 20370471A/T
6-3: 20370474G/A
6-3: 20370501G/A
6-3: 20370519C/T
6-3: 20370567A/G
6-3: 20370570A/G
6-3: 20370786A/G
6-3: 20370864T/G
6-3: 20370918T/C
6-3: 20370939A/G
6-3: 20370972T/C
6-3: 20371713T/C
6-3: 20380649T/A
6-3: 20410433T/G
6-3: 20429821A/G
6-3: 20437540T/G
6-3: 20437626C/G
6-3: 20441355A/T
6-3: 20493654C/A
6-3: 20540487A/T
6-3: 20560829C/T
6-3: 20572561C/T
6-3: 20581473G/A
6-3: 20583724G/A
6-3: 20606403T/C
6-3: 20609563C/A
6-3: 20609616A/T
6-3: 20609700G/A
6-3: 20609925A/T
6-3: 20611114A/G
6-3: 20611682T/G
6-3: 20612466A/T
6-3: 20613305A/T
6-3: 20613406G/T
6-3: 20621988A/C
6-3: 20622951C/T
6-3: 20631595C/T
6-3: 20636918C/T
6-3: 20670782T/C
6-3: 20714015A/G
6-3: 20758815T/C
6-3: 20775201C/A
6-3: 20794021G/A
6-3: 20922888G/A
6-3: 20925110A/T
6-3: 20925790A/G
6-3: 20939433C/T
6-3: 20972476A/G
6-3: 21011202C/T
6-3: 21022054T/C
6-3: 21071798G/C
6-3: 21107698C/G
6-3: 21156715C/T
6-3: 21161063T/C
6-3: 21198851C/G
6-3: 21213933G/T
6-3: 21227241G/A
6-3: 21240534G/T
6-3: 21298124C/T
6-3: 21329335G/A
6-3: 21343224G/T
6-3: 21366684G/T
6-3: 21376004T/G
6-3: 21446601G/C
6-3: 21449457C/T
6-3: 21449458C/T
6-3: 21459930T/A
6-3: 21482485G/A
6-3: 21488989A/T
6-3: 21495588A/C
6-3: 21508807T/A
6-3: 21524338T/A
6-3: 21537395T/A
6-3: 21566344T/C
6-3: 21574641T/A
6-3: 21577940C/T
6-3: 21603668T/G
6-3: 21654125G/T
6-3: 21660009T/A
6-3: 21660831A/T
6-3: 21662192A/G
6-3: 21662330A/G
6-3: 21662954C/A
6-3: 21662962T/C
6-3: 21682881A/C
6-3: 21722659T/G
6-3: 21723245A/G
6-3: 21723509T/C
6-3: 21723581A/G
6-3: 21743877T/G
6-3: 21763009T/C
6-3: 21866891T/A
6-3: 21881608T/G
6-3: 21897265C/T
6-3: 21951923G/A
6-3: 21951957A/G
6-3: 22019365C/T
6-3: 22622281A/G
6-3: 23104916C/T
6-3: 23380102C/T
6-3: 23658180C/A
6-3: 23660970G/A
6-3: 23779837G/A
6-3: 23888454C/G
6-3: 24282542A/G
6-3: 24413171C/T
6-3: 24491550C/T
6-3: 24605512C/T
6-3: 24605581C/T
6-3: 24656091T/G
6-3: 24690942A/G
6-3: 24705923A/G
6-3: 26084863C/T
6-3: 26092524A/T
6-3: 26113045T/A
6-3: 26151052A/C
6-3: 26156965C/A
6-3: 26187452T/C
6-3: 26194723T/C
6-3: 26329636C/A
6-3: 26335694A/G
6-3: 26383507C/T
6-3: 26634171G/C
6-3: 26646461T/C
6-3: 26680425A/G
6-3: 26705431G/A
6-3: 26852681G/A
6-3: 26967446G/A
6-3: 27201538G/C
6-3: 27218610G/A
6-3: 27334575T/A
6-3: 27676516G/A
6-3: 27724143A/G
6-3: 27881188T/C
6-3: 27956862C/T
6-3: 28032842C/A
6-3: 28040545C/T
6-3: 28050559A/C
6-3: 28071652C/T
6-3: 28246566C/T
6-3: 28304845A/G
6-3: 28323720C/T
6-3: 28388999T/G
6-3: 28457375T/A
6-3: 28733831G/A
6-3: 28798045A/C
6-3: 28810177T/A
6-3: 28820590A/G
6-3: 28939484G/A
6-3: 28949339T/A
6-3: 28949443C/G
6-3: 29055919A/G
6-3: 29643547C/T
6-3: 29799811A/C
6-3: 29801942C/T
6-3: 29803212C/T
6-3: 29879615G/A
6-3: 29912296A/T
6-3: 29963778G/T
6-3: 29980614A/T
6-3: 29981762T/A
6-3: 30018776C/T
6-3: 30019380T/C
6-3: 30045626G/A
6-3: 30051151A/G
6-3: 30051293T/A
6-3: 30052189C/T
6-3: 30052438A/G
6-3: 30052651T/C
6-3: 30052938C/A
6-3: 30055937G/T
6-3: 30079813C/T
6-3: 30105309G/A
6-3: 30116403C/T
6-3: 30132649A/G
6-3: 30153308A/G
6-3: 30159174A/G
6-3: 30159189G/A
6-3: 30183719G/A
6-3: 30196296C/T
6-3: 30212495A/C
6-3: 30230618T/C
6-3: 30231553C/A
6-3: 30232049G/A
6-3: 30249928G/A
6-3: 30341681C/A
6-3: 30344617C/T
6-3: 30352846A/T
6-3: 30362876A/G
6-3: 30363119C/T
6-3: 30363749G/C
6-3: 30365361C/A
6-3: 30365491A/G
6-3: 30366191G/A
6-3: 30366499T/C
6-3: 30368085T/A
6-3: 30368169C/T
6-3: 30368225T/C
6-3: 30387800A/G
6-3: 30414384T/G
6-3: 30428507C/T
6-3: 30442526T/C
6-3: 30443068C/T
6-3: 30443258A/G
6-3: 30443363G/A
6-3: 30443464C/T
6-3: 30443669T/C
6-3: 30443706T/C
6-3: 30459286T/G
6-3: 30462844A/G
6-3: 30462969A/G
6-3: 30463863T/C
6-3: 30465257A/G
6-3: 30468230T/A
6-3: 30468968T/A
6-3: 30469524A/G
6-3: 30474032C/A
6-3: 30474087A/G
6-3: 30474090A/G
6-3: 30489797C/G
6-3: 30490672A/C
6-3: 30490833T/A
6-3: 30490916G/A
6-3: 30491125C/G
6-3: 30491476T/G
6-3: 30505489A/G
6-3: 30525567C/T
6-3: 30526960G/C
6-3: 30634243G/T
6-3: 30711645C/T
6-3: 30799190A/T
6-3: 30819102T/C
6-3: 30826222G/A
6-3: 30829719G/T
6-3: 30831669T/C
6-3: 30854668T/C
6-3: 30903857G/T
6-3: 30914151A/G
6-3: 30961315A/G
6-3: 30973296A/G
6-3: 30987637T/A
6-3: 30998466A/G
6-3: 31009421C/T
6-3: 31036038C/G
6-3: 31071977C/T
6-3: 31084018G/T
6-3: 31159928C/T
6-3: 31241118A/G
6-3: 31304838C/T
6-3: 31359967G/A
6-3: 31448364T/C
6-3: 31456579T/C
6-3: 31482282C/T
6-3: 31488386T/C
6-3: 31505904A/G
6-3: 31535181C/G
6-3: 31559450T/C
6-3: 31601071C/T
6-3: 31630934T/G
6-3: 31667067T/A
6-3: 31667239G/T
6-3: 31696188T/A
6-3: 31696479G/A
6-3: 31696712G/T
6-3: 31748377G/C
6-3: 31881280T/G
6-3: 31971455T/C
6-3: 32014842T/A
6-3: 32053088G/A
6-3: 32090070T/C
6-3: 32157299G/A
6-3: 32185656A/T
6-3: 32256807C/T
6-3: 32361403A/C
6-3: 32441970G/T
6-3: 32507447A/T
6-3: 32533591C/T
6-3: 32560742G/A
6-3: 32675471C/A
6-3: 32919749T/G
6-3: 32938591A/G
6-3: 32957974C/T
6-3: 32984060T/A
6-3: 33081959C/A
6-3: 33133865A/T
6-3: 33193315G/A
6-3: 33304036G/T
6-3: 33360057C/G
6-3: 33364478A/C
6-3: 33406006T/G
6-3: 33416201G/T
6-3: 33473520T/C
6-3: 33488978G/C
6-3: 33567787A/G
6-3: 33635633A/T
6-3: 33656806A/G
6-3: 33656924A/G
6-3: 33657037C/G
6-3: 33657040G/A
6-3: 33657354A/G
6-3: 33657557G/A
6-3: 33664066T/C
6-3: 33773289T/A
6-3: 33802953C/T
6-3: 33803486A/G
6-3: 33826635C/T
6-3: 33872209T/C
6-3: 34033877C/G
6-3: 34051693T/C
6-3: 34100104C/G
6-3: 34148115C/T
6-3: 34277034A/C
6-3: 34329341C/T
6-3: 34589344C/T
6-3: 34660183T/C
6-3: 34772250A/T
6-3: 34846129C/T
6-3: 34928566C/A
6-3: 34963975G/C
6-3: 35016022G/A
6-3: 35105204G/T
6-3: 35120404A/T
6-3: 35184072T/C
6-3: 35212099G/A
6-3: 35364759T/C
6-3: 35378559T/C
6-3: 35401164A/T
6-3: 35476925T/A
6-3: 35509671C/G
6-3: 35545198G/A
6-3: 35584338C/A
6-3: 35588130G/A
6-3: 35883562G/A
6-3: 35938028C/T
6-3: 35963409C/T
6-3: 35983742C/T
6-3: 35985399G/T
6-3: 35985553C/T
6-3: 36003284A/G
6-3: 36029741A/T
6-3: 36087071A/G
6-3: 36091134C/G
6-3: 36118768G/A
6-3: 36133336T/A
6-3: 36151527T/C
6-3: 36162536C/T
6-3: 36162706C/T
6-3: 36181767C/T
6-3: 36271932C/G
6-3: 36307331C/T
6-3: 36338625A/T
6-3: 36413612C/T
6-3: 36447467G/C
6-3: 36601941C/T
6-3: 36679379C/G
6-3: 36793704A/T
6-3: 36852040G/C
6-3: 36863417C/A
6-3: 36863484C/G
6-3: 36884007C/A
6-3: 36897302C/G
6-3: 36920583A/C
6-3: 36949779T/G
6-3: 36974613A/C
6-3: 37041764A/T
6-3: 37090793C/T
6-3: 37217028G/A
6-3: 37241073C/A
6-3: 37290996C/T
6-3: 37293581T/C
6-3: 37297506T/C
6-3: 37319453C/T
6-3: 37319484T/C
6-3: 37320605C/T
6-3: 37321121G/A
6-3: 37373501A/T
6-3: 37390605A/T
6-3: 37419387C/A
6-3: 37442726T/C
6-3: 37463092G/A
6-3: 37463500T/A
6-3: 37464083C/T
6-3: 37464096C/T
6-3: 37489013T/C
6-3: 37489197C/A
6-3: 37501138A/G
6-3: 37588415C/T
6-3: 37605742C/T
6-3: 37630203T/C
6-3: 37714238T/A
6-3: 37783459A/G
6-3: 37817878T/G
6-3: 37867384G/A
6-3: 37962361G/A
6-3: 37991660G/A
6-3: 38043163A/G
6-3: 38204518T/C
6-3: 38220036G/T
6-3: 38221617A/G
6-3: 38221674C/T
6-3: 38223897T/C
6-3: 38224276G/A
6-3: 38224618T/A
6-3: 38224827A/G
6-3: 38224831G/A
6-3: 38240026A/T
6-3: 38249601T/C
6-3: 38259545T/G
6-3: 38309048G/A
6-3: 38322096C/T
6-3: 38332162C/T
6-3: 38342972G/C
6-3: 38343355G/C
6-3: 38343606C/T
6-3: 38344562C/T
6-3: 38344812A/T
6-3: 38351178A/G
6-3: 38351372A/T
6-3: 38352443T/C
6-3: 38352600C/T
6-3: 38352641C/T
6-3: 38352658C/T
6-3: 38352912T/A
6-3: 38353628T/C
6-3: 38353730C/A
6-3: 38356722A/T
6-3: 38356890T/C
6-3: 38369760C/T
6-3: 38390626A/G
6-3: 38403035A/G
6-3: 38415194T/G
6-3: 38416989A/T
6-3: 38430892G/A
6-3: 38520940A/G
6-3: 38535261T/A
6-3: 38556232T/A
6-3: 38570043C/G
6-3: 38668078A/G
6-3: 38708351A/G
6-3: 38779796G/A
6-3: 38782628A/G
6-3: 38876461G/A
6-3: 38893586T/G
6-3: 38957069G/A
6-3: 38972641A/G
6-3: 39071114T/A
6-3: 39124598C/A
6-3: 39213969G/A
6-3: 39236924T/C
6-3: 39253681T/A
6-3: 39289181A/G
6-3: 39336985T/C
6-3: 39363741C/T
6-3: 39454803A/G
6-3: 39459380T/A
6-3: 39459492G/A
6-3: 39459723C/T
6-3: 39473132A/G
6-3: 39481925T/C
6-3: 39482017A/G
6-3: 39541419C/G
6-3: 39587733T/C
6-3: 39587813A/T
6-3: 39623153G/A
6-3: 39651593C/T
6-3: 39705792A/G
6-3: 39755036T/G
6-3: 39768211A/G
6-3: 39789269T/A
6-3: 39789972A/C
6-3: 39828502T/C
6-3: 39844023A/T
6-3: 39885751G/A
6-3: 39927185C/A
6-3: 40033772A/G
6-3: 40052369C/T
6-3: 40107822A/T
6-3: 40168562C/T
6-3: 40281185T/A
6-3: 40319059C/A
6-3: 40386146A/T
6-3: 40453637C/T
6-3: 40456495T/G
6-3: 40457055A/T
6-3: 40466429G/A
6-3: 40466591G/A
6-3: 40479359A/T
6-3: 40524666C/T
6-3: 40562381G/A
6-3: 40642253T/C
6-3: 40674379C/T
6-3: 40704209G/A
6-3: 40759686A/T
6-3: 40782578C/T
6-3: 40871254C/T
6-3: 40878616C/T
6-3: 40898493A/T
6-3: 40910061T/A
6-3: 40931306C/T
6-3: 40974293G/A
6-3: 41054638C/T
6-3: 41054906C/G
6-3: 41076661A/T
6-3: 41087005G/A
6-3: 41135277C/T
6-3: 41271765C/T
6-3: 41353504C/A
6-3: 41381634G/A
6-3: 41419465T/C
6-3: 41512645A/G
6-3: 41524432G/A
6-3: 41551994G/C
6-3: 41555791C/T
6-3: 41555814G/C
6-3: 41555833T/C
6-3: 41559290G/C
6-3: 41584996T/A
6-3: 41624733A/G
6-3: 41669664A/G
6-3: 41687758A/T
6-3: 41779893A/G
6-3: 41824947C/A
6-3: 41878387A/T
6-3: 42034277C/G
6-3: 42105350A/C
6-3: 42222291T/A
6-3: 42224932G/T
6-3: 42245110T/A
6-3: 42261992C/A
6-3: 42411373A/C
6-3: 42468700G/A
6-3: 42548625G/A
6-3: 42597529C/T
6-3: 42613857C/G
6-3: 42656671C/T
6-3: 42667423A/T
6-3: 42827575G/A
6-3: 42911952C/A
6-3: 42993107A/G
6-3: 43103554A/C
6-3: 43114304A/T
6-3: 43128706A/G
6-3: 43177949T/C
6-3: 43274518A/T
6-3: 43284671T/A
6-3: 43297512A/T
6-3: 43318504C/A
6-3: 43338165G/T
6-3: 43360235C/T
6-3: 43378386C/T
6-3: 43406532C/T
6-3: 43446494C/T
6-3: 43462049A/G
6-3: 43524091C/T
6-3: 43542405T/A
6-3: 43544726G/T
6-3: 43545070C/T
6-3: 43545812A/T
6-3: 43567542A/T
6-3: 43570646T/C
6-3: 43579498A/G
6-3: 43580579A/T
6-3: 43588397T/A
6-3: 43616837G/A
6-3: 43626989G/A
6-3: 43659261C/A
6-3: 43718524T/C
6-3: 43737083T/A
6-3: 43749187A/T
6-3: 43750783A/G
6-3: 43768940G/A
6-3: 43780173C/T
6-3: 43850839C/T
6-3: 43870212C/T
6-3: 43944996C/T
6-3: 43982753C/T
6-3: 43991068G/A
6-3: 43993815C/G
6-3: 44015238A/T
6-3: 44046564T/C
6-3: 44344359T/C
6-3: 44375981T/C
6-3: 44387111G/A
6-3: 44460318G/A
6-3: 44616582A/T
6-3: 44643073T/C
6-3: 44703967T/C
6-3: 44738254C/T
6-3: 44750425A/G
6-3: 44854797G/A
6-3: 44863053G/T
6-3: 44884476T/C
6-3: 44919174A/G
6-3: 44962577A/T
6-3: 44976821C/T
6-3: 44987099T/C
6-3: 44991532A/C
6-3: 45019948A/G
6-3: 45188379A/G
6-3: 45260918T/C
6-3: 45260926C/T
6-3: 45260937C/T
6-3: 45262685A/G
6-3: 45265778T/C
6-3: 45265968G/A
6-3: 45266528T/A
6-3: 45275457A/G
6-3: 45275619G/C
6-3: 45275764C/G
6-3: 45279019C/T
6-3: 45279095C/T
6-3: 45317221G/A
6-3: 45317886T/C
6-3: 45318054C/A
6-3: 45318171T/C
6-3: 45466312T/C
6-3: 45526079G/T
6-3: 45544091T/A
6-3: 45556213C/T
6-3: 45558367C/G
6-3: 45571714C/A
6-3: 45610746C/A
6-3: 45647200C/A
6-3: 45687675A/T
6-3: 45732700T/A
6-3: 45777333G/A
6-3: 45783657T/A
6-3: 45785973T/C
6-3: 45851670C/T
6-3: 45891917G/A
6-3: 45907440C/T
6-3: 45926209T/G
6-3: 45938049A/G
6-3: 45948590A/G
6-3: 45960153T/G
6-3: 46007250T/C
6-3: 46018153C/T
6-3: 46032615T/G
6-3: 46043482A/C
6-3: 46071972C/T
6-3: 46086348G/A
6-3: 46109454G/T
6-3: 46120691G/A
6-3: 46121793C/T
6-3: 46154996T/A
6-3: 46182250G/C
6-3: 46199166T/G
6-3: 46205693G/A
6-3: 46245153A/C
6-3: 46322475T/A
6-3: 46352965G/A
6-3: 46374950C/A
6-3: 46529037G/A
6-3: 46608114C/T
6-3: 46609726A/G
6-3: 46632238C/T
6-3: 46695248G/A
6-3: 46714923A/C
6-3: 46730563T/A
6-3: 46733355G/A
6-3: 46738185G/T
6-3: 46756129A/T
6-3: 46854470A/T
6-3: 46871371A/G
6-3: 46889616A/T
6-3: 46902896C/T
6-3: 46916992A/G
6-3: 46927443T/C
6-3: 46953206G/A
6-3: 46968676G/T
6-3: 47017134A/T
6-3: 47021914G/A
6-3: 47022017T/C
6-3: 47022263C/T
6-3: 47037570T/C
6-3: 47057028A/C
6-3: 47060575G/T
6-3: 47067218A/G
6-3: 47090545C/T
6-3: 47090867C/A
6-3: 47092160C/T
6-3: 47098328T/G
6-3: 47098431A/G
6-3: 47098996A/G
6-3: 47099123A/T
6-3: 47099359G/A
6-3: 47099499C/T
6-3: 47099804T/A
6-3: 47099909A/G
6-3: 47100041C/T
6-3: 47100164T/G
6-3: 47100553T/C
6-3: 47107004G/A
6-3: 47107213A/T
6-3: 47107377A/G
6-3: 47107559T/C
6-3: 47107560A/C
6-3: 47107823G/A
6-3: 47108370A/G
6-3: 47108448G/T
6-3: 47110963T/C
6-3: 47141426C/A
6-3: 47159314C/A
6-3: 47183701G/A
6-3: 47194919G/T
6-3: 47195171G/A
6-3: 47195288A/G
6-3: 47195896A/G
6-3: 47209996C/T
6-3: 47210200T/G
6-3: 47234294T/C
6-3: 47246880C/T
6-3: 47285516T/G
6-3: 47305990C/T
6-3: 47326224T/G
6-3: 47330280G/A
6-3: 47338365G/A
6-3: 47359670C/A
6-3: 47373944T/A
6-3: 47391891G/T
6-3: 47402141A/G
6-3: 47417919A/T
6-3: 47516812C/T
6-3: 47553620A/C
6-3: 47576200T/G
6-3: 47581098T/C
6-3: 47588968T/C
6-3: 47599735C/T
6-3: 47610571T/C
6-3: 47610725T/A
6-3: 47610995T/G
6-3: 47622676T/A
6-3: 47637181G/A
6-3: 47652183G/T
6-3: 47653354G/A
6-3: 47654694C/A
6-3: 47663591G/A
6-3: 47670082A/T
6-3: 47670665A/G
6-3: 47674762T/A
6-3: 47709136G/A
6-3: 47712724G/A
6-3: 47712732C/T
6-3: 47733660C/T
6-3: 47740816A/T
6-3: 47775363G/A
6-3: 47784916A/G
6-3: 47784973A/G
6-3: 47786283T/C
6-3: 47786766A/T
6-3: 47787643T/C
6-3: 47787799A/G
6-3: 47787907A/C
6-3: 47796935C/T
6-3: 47807550C/G
6-3: 47813546A/T
6-3: 47838646C/T
6-3: 47882080G/A
6-3: 47892402A/G
6-3: 47892408T/.
6-3: 47892458A/.
6-3: 47892468C/.
6-3: 47892509G/.
6-3: 47892512G/.
6-3: 47892551A/.
6-3: 47913431G/A
6-3: 47915766G/A
6-3: 47926145A/G
6-3: 47938942T/A
6-3: 47967526T/C
6-3: 47979588C/G
6-3: 47992372A/C
6-3: 47996322G/A
6-3: 48012996A/T
6-3: 48040939T/C
6-3: 48115214G/A
6-3: 48181781C/A
6-3: 48233570G/A
6-3: 48270406T/A
6-3: 48286291C/T
6-3: 48378444G/A
6-3: 48403473T/G
6-3: 48434186C/T
6-3: 48473967A/T
6-3: 48596413A/G
6-3: 48597671T/C
6-3: 48607760C/A
6-3: 48643907T/A
6-3: 48733022G/A
6-3: 48796755C/T
6-3: 48822792A/G
6-3: 48854707T/A
6-3: 48899312C/T
6-3: 48956692C/T
6-3: 48979287G/A
6-3: 49023283A/C
6-3: 49033540C/T
6-3: 49033820T/A
6-3: 49033991C/A
6-3: 49034044C/T
6-3: 49034089T/C
6-3: 49034258G/T
6-3: 49034377G/A
6-3: 49034491C/T
6-3: 49034596T/G
6-3: 49034615G/A
6-3: 49034655T/G
6-3: 49034932T/G
6-3: 49035206G/C
6-3: 49035320G/A
6-3: 49035970G/A
6-3: 49036073G/A
6-3: 49044954A/G
6-3: 49094873A/C
6-3: 49161466G/A
6-3: 49205780T/A
6-3: 49246721T/A
6-3: 49296846T/C
6-3: 49356665C/T
6-3: 49368847T/C
6-3: 49399012C/T
6-3: 49542130C/T
6-3: 49569732G/A
6-3: 49676110G/A
6-3: 49687342C/T
6-3: 49856953T/C
6-3: 49939421C/T
6-3: 49964039T/C
6-3: 50035954C/A
6-3: 50088020C/T
6-3: 50283082A/T
6-3: 50439888G/C
6-3: 50566417T/A
6-3: 50948494T/G
6-3: 51055165G/A
6-3: 51087579T/C
6-3: 51103392G/T
6-3: 51211799G/A
6-3: 51255418C/T
6-3: 51275427T/C
6-3: 51472834T/A
6-3: 51638049C/G
6-3: 51813864T/C
6-3: 51877460T/A
6-3: 51967118A/G
6-3: 52373396A/G
6-3: 52447440A/G
6-3: 52472790A/T
6-3: 52505297T/G
6-3: 52567414C/T
6-3: 52665949A/T
6-3: 52699947G/A
6-3: 52723103T/C
6-3: 52806596C/T
6-3: 52989717C/T
6-3: 53151799C/G
6-3: 53187368A/G
6-3: 53207929A/T
6-3: 53545312G/A
6-3: 53689719G/A
6-3: 53774638G/A
6-3: 53861169T/C
6-3: 53956480C/A
6-3: 53977439T/C
6-3: 54260178A/G
6-3: 54319422G/A
6-3: 54444333A/G
6-3: 54594062A/T
6-3: 54735110C/T
6-3: 54898798A/G
6-3: 54971835T/C
6-3: 55051616C/T
6-3: 55207170A/G
6-3: 55358516T/A
6-3: 55501055A/G
6-3: 55579973A/T
6-3: 55711240C/T
6-3: 55718884T/C
6-3: 55748493C/A
6-3: 55761329A/C
6-3: 56024275A/T
6-3: 56078216C/T
6-3: 56206259G/T
6-3: 56259163C/T
6-3: 56329671C/T
6-3: 56461305G/A
6-3: 56476577C/A
6-3: 56609462G/A
6-3: 56734094C/T
6-3: 56881136T/A
6-3: 57221269C/T
6-3: 57228598G/A
6-3: 57249494G/A
6-3: 57402204C/T
6-3: 57418594G/T
6-3: 57434558T/C
6-3: 57580866G/A
6-3: 57590852A/G
6-3: 57668295T/A
6-3: 57699070A/G
6-3: 57753410T/C
6-3: 57817702C/T
6-3: 57885731G/C
6-3: 58047869A/C
6-3: 58092064G/T
6-3: 58092864A/G
6-3: 58120265A/G
6-3: 58134059G/A
6-3: 58168889T/A
6-3: 58217431C/T
6-3: 58243115C/G
6-3: 58258890G/A
6-3: 58435068T/G
6-3: 58486915G/A
6-3: 58525912A/T
6-3: 58575384A/T
6-3: 58605555T/A
6-3: 58804244G/A
6-3: 58860645C/T
6-3: 58884550T/C
6-3: 58942317T/C
6-3: 58953799C/T
6-3: 58957436A/G
6-3: 58957729A/G
6-3: 58989474T/C
6-3: 59042808G/T
6-3: 59152076C/A
6-3: 59163069T/A
6-3: 59245619T/A
6-3: 59292407G/A
6-3: 59311072G/A
6-3: 59323980C/T
6-3: 59326572C/T
6-3: 59337880C/G
6-3: 59348308A/G
6-3: 59357257G/T
6-3: 59357270G/C
6-3: 59359582C/G
6-3: 59500917G/T
6-3: 59570979A/T
6-3: 59715462C/T
6-3: 59783044G/T
6-3: 59784525C/T
6-3: 59784603T/C
6-3: 59785783C/A
6-3: 59787136A/C
6-3: 59869625C/T
6-3: 59924663C/A
6-3: 59929040T/A
6-3: 59973995T/A
6-3: 60065702A/G
6-3: 60111884T/C
6-3: 60131385C/T
6-3: 60193767G/A
6-3: 60235718C/T
6-3: 60253667T/G
6-3: 60267070C/T
6-3: 60345530G/A
6-3: 60379457C/T
6-3: 60408548T/G
6-3: 60471849T/C
6-3: 60500009G/A
6-3: 60526816C/T
6-3: 60529437G/A
6-3: 60532068A/G
6-3: 60543645G/A
6-3: 60565401T/C
6-3: 60568235T/C
6-3: 60675078G/A
6-3: 60733435C/T
6-3: 60782197A/T
6-3: 60795296G/A
6-3: 60928200C/T
6-3: 61085079C/T
6-3: 61103846A/G
6-3: 61147171G/A
6-3: 61148418G/A
6-3: 61161344C/A
6-3: 61190748T/A
6-3: 61271633A/T
6-3: 61339336C/A
6-3: 61437222T/A
6-3: 61504828C/T
6-3: 61588054A/G
6-3: 61615106A/G
6-3: 61637601T/C
6-3: 61702807A/G
6-3: 61754126T/A
6-3: 61798521T/G
6-3: 61826831G/T
6-3: 61856704G/C
6-3: 61860339A/T
6-3: 61920812G/A
6-3: 61933945T/C
6-3: 62001079G/A
6-3: 62012561A/G
6-3: 62074003A/G
6-3: 62084723G/A
6-3: 62095727A/T
6-3: 62189356T/G
6-3: 62234964G/A
6-3: 62235257G/C
6-3: 62240700G/A
6-3: 62247049G/A
6-3: 62329077T/C
6-3: 62355057C/T
6-3: 62356713A/G
6-3: 62386267C/T
6-3: 62411285T/C
6-3: 62429261T/A
6-3: 62458500G/C
6-3: 62517614T/A
6-3: 62714705C/T
6-3: 62719735A/C
6-3: 62784580C/A
6-3: 62801573C/G
6-3: 62883781T/A
6-3: 62932004A/T
6-3: 62974917C/G
6-3: 62985784G/T
6-3: 63277201C/T
6-3: 63293348G/A
6-3: 63300962C/T
6-3: 63315912T/C
6-3: 63345007G/A
6-3: 63416988G/A
6-3: 63432661C/T
6-3: 63447064G/A
6-3: 63463347C/G
6-3: 63463348G/C
6-3: 63534191A/G
6-3: 63685122G/A
6-3: 63698167G/T
6-3: 63708781C/T
6-3: 63719858G/T
6-3: 63730016G/C
6-3: 63731488G/T
6-3: 63783888C/G
6-3: 63823539G/A
6-3: 63846834A/T
6-3: 63869793G/T
6-3: 63951535A/T
6-3: 63964354G/T
6-3: 64028037G/A
6-3: 64038071C/T
6-3: 64038691T/C
6-3: 64051984T/C
6-3: 64069167G/A
6-3: 64091081G/A
6-3: 64132557A/C
6-3: 64145174A/T
6-3: 64204678A/C
6-3: 64271240C/A
6-3: 64370752G/A
6-3: 64441331A/G
6-3: 64645464C/T
6-3: 64773745T/C
6-3: 64928183A/T
6-3: 64928430T/C
6-3: 64928555G/T
6-3: 64928808G/A
6-3: 64929062A/T
6-3: 65026171T/A
6-3: 65075769A/G
6-3: 65105117T/A
6-3: 65165186C/T
6-3: 65184659A/T
6-3: 65331969C/A
6-3: 65346512C/T
6-3: 65475925C/T
6-3: 65487748G/A
6-3: 65504836C/T
6-3: 65516712T/A
6-3: 65540513T/G
6-3: 65564494A/T
6-3: 65628877T/C
6-3: 65650246A/G
6-3: 65947005G/A
6-3: 66115885G/A
6-3: 66117553C/A
6-3: 66184215A/T
6-3: 66288350G/A
6-3: 66370512C/T
6-3: 66400517T/C
6-3: 66580312A/G
6-3: 66700526G/A
6-3: 66730710A/T
6-3: 66774136T/A
6-3: 66885473C/A
6-3: 66886719T/C
6-3: 67023873A/G
6-3: 67028380A/G
6-3: 67043787C/T
6-3: 67062944A/T
6-3: 67064247G/A
6-3: 67088239A/G
6-3: 67144764C/T
6-3: 67192060A/C
6-3: 67237864A/T
6-3: 67275015A/T
6-3: 67395631T/A
6-3: 67477998T/A
6-3: 67575114C/T
6-3: 67681402A/G
6-3: 67850926G/A
6-3: 67869217A/T
6-3: 67913835C/A
6-3: 67943923T/C
6-3: 67969121C/T
6-3: 68005198G/A
6-3: 68005787G/T
6-3: 68006172G/T
6-3: 68022243G/C
6-3: 68042853A/G
6-3: 68140908C/T
6-3: 68174617G/A
6-3: 68188923A/G
6-3: 68214515G/T
6-3: 68292052A/T
6-3: 68693288G/T
6-3: 68772623G/A
6-3: 68828995T/A
6-3: 68879790T/C
6-3: 68913717A/G
6-3: 68915024C/A
6-3: 68915073A/T
6-3: 68941924G/C
6-3: 69000104C/A
6-3: 69058220G/A
6-3: 69077366T/A
6-3: 69087519T/C
6-3: 69249530A/C
6-3: 69297779C/T
6-3: 69331866C/T
6-3: 69368058G/A
6-3: 69533905T/G
6-3: 69639618T/G
6-3: 69639653T/C
6-3: 69642326T/C
6-3: 69678443T/C
6-3: 69712714T/C
6-3: 69730966A/C
6-3: 69737330G/T
6-3: 69799661A/T
6-3: 69836244A/G
6-3: 69952831T/A
6-3: 69982939T/C
6-3: 69986689C/T
6-3: 69999920T/C
6-3: 70016572G/T
6-3: 70030030T/G
6-3: 70065519C/T
6-3: 70075507A/G
6-3: 70075848A/G
6-3: 70109997C/T
6-3: 70124348A/G
6-3: 70156415A/G
6-3: 70172476C/T
6-3: 70184155G/A
6-3: 70373115A/G
6-3: 70390029T/A
6-3: 70420292T/C
6-3: 70443136G/A
6-3: 70454779T/C
6-3: 70488314G/A
6-3: 70529799G/A
6-3: 70580094G/A
6-3: 70605725C/T
6-3: 70720563A/G
6-3: 70736956T/C
6-3: 70737075A/G
6-3: 70737099A/T
6-3: 70738144C/A
6-3: 70738182G/A
6-3: 70738468T/C
6-3: 70752687T/C
6-3: 70785013A/T
6-3: 70785528G/C
6-3: 70787188A/T
6-3: 70788506C/T
6-3: 70788636C/T
6-3: 70788933A/G
6-3: 70789051T/C
6-3: 70789086A/G
6-3: 70789345A/G
6-3: 70789434T/C
6-3: 70789603T/C
6-3: 70789647A/T
6-3: 70799002G/A
6-3: 70812462C/T
6-3: 70823552G/A
6-3: 70998715G/T
6-3: 71297016T/A
6-3: 71464771T/C
6-3: 71515865T/G
6-3: 71600476G/A
6-3: 71633865A/G
6-3: 71688515C/T
6-3: 71720478A/C
6-3: 71735704G/C
6-3: 71767716A/T
6-3: 71802225A/G
6-3: 71817648C/T
6-3: 71861826A/G
6-3: 71876991C/G
6-3: 72053879T/G
6-3: 72129582G/A
6-3: 72281523A/C
6-3: 72352973C/T
6-3: 72359435T/C
6-3: 72399259T/C
6-3: 72407296G/A
6-3: 72437964T/G
6-3: 72529095G/A
6-3: 72561190C/G
6-3: 72577025G/A
6-3: 72587027A/T
6-3: 72703798G/C
6-3: 72747825G/A
6-3: 72751303C/A
6-3: 72774174A/G
6-3: 72885411G/T
6-3: 72954107T/C
6-3: 72972056C/T
6-3: 73044359A/T
6-3: 73059339T/C
6-3: 73095343A/G
6-3: 73112091T/G
6-3: 73205081G/A
6-3: 73248451G/A
6-3: 73258491A/C
6-3: 73291419A/T
6-3: 73306578C/T
6-3: 73341106C/T
6-3: 73387851A/G
6-3: 73420716T/C
6-3: 73472498C/T
6-3: 73537138G/A
6-3: 73559930T/C
6-3: 73587923G/A
6-3: 73645168C/T
6-3: 73657439T/C
6-3: 73662865C/T
6-3: 73693714C/T
6-3: 73769942C/T
6-3: 73800747A/T
6-3: 73849329C/T
6-3: 73860493T/C
6-3: 73885161A/G
6-3: 73934246C/T
6-3: 74037428A/G
6-3: 74198939G/A
6-3: 74210482C/T
6-3: 74254997A/G
6-3: 74278990A/G
6-3: 74289147C/T
6-3: 74303492G/A
6-3: 74331281G/A
6-3: 74346069C/T
6-3: 74363277T/C
6-3: 74425660C/T
6-3: 74581923T/C
6-3: 74592127T/A
6-3: 74604559A/G
6-3: 74618487A/G
6-3: 74651911A/C
6-3: 74671525C/T
6-3: 74709894C/T
6-3: 74737820A/G
6-3: 74749842A/C
6-3: 74780151A/G
6-3: 74796534G/A
6-3: 74835492T/C
6-3: 74869635G/A
6-3: 74982945C/T
6-3: 75023232T/C
6-3: 75032241T/A
6-3: 75048585C/T
6-3: 75074697A/G
6-3: 75107003C/T
6-3: 75133241T/C
6-3: 75144368T/C
6-3: 75184571G/A
6-3: 75203333C/T
6-3: 75209827C/A
6-3: 75209971G/C
6-3: 75215723C/T
6-3: 75226555G/T
6-3: 75259615A/T
6-3: 75316202A/G
6-3: 75333995C/T
6-3: 75340623T/C
6-3: 75341057T/G
6-3: 75341158T/A
6-3: 75381743A/G
6-3: 75421314C/T
6-3: 75452259T/G
6-3: 75452531T/C
6-3: 75477925C/A
6-3: 75488706C/T
6-3: 75507581G/A
6-3: 75649823A/T
6-3: 75650085G/A
6-3: 75697411G/C
6-3: 75742520C/T
6-3: 75843818A/G
6-3: 75856766A/G
6-3: 75856995T/C
6-3: 75873589G/T
6-3: 75880664A/G
6-3: 75893616A/C
6-3: 75913763G/A
6-3: 75968204G/A
6-3: 75999560T/A
6-3: 76155960A/G
6-3: 76185152T/G
6-3: 76209877T/C
6-3: 76238243A/G
6-3: 76274595C/G
6-3: 76327611T/A
6-3: 76328905T/A
6-3: 76329273T/A
6-3: 76403099A/G
6-3: 76437070T/C
6-3: 76447977C/A
6-3: 76486146C/A
6-3: 76584967G/T
6-3: 76623192T/G
6-3: 76703301A/C
6-3: 76750299G/T
6-3: 76846050T/C
6-3: 76856450A/C
6-3: 76943157G/C
6-3: 76962699T/C
6-3: 76968855T/C
6-3: 76969035C/T
6-3: 76969146G/A
6-3: 76969151T/C
6-3: 76970060G/A
6-3: 76975965A/C
6-3: 77006077A/T
6-3: 77019067G/A
6-3: 77032006T/C
6-3: 77051071C/A
6-3: 77064310A/C
6-3: 77069429C/A
6-3: 77076948G/C
6-3: 77082234A/T
6-3: 77092606A/T
6-3: 77128847G/A
6-3: 77143725G/A
6-3: 77190824C/A
6-3: 77203514T/C
6-3: 77241571A/T
6-3: 77313032A/T
6-3: 77349483T/C
6-3: 77370968G/A
6-3: 77455443T/C
6-3: 77505118T/G
6-3: 77530867G/C
6-3: 77536144G/A
6-3: 77550126A/T
6-3: 77676958C/A
6-3: 77760484G/C
6-3: 77856614G/A
6-3: 77866204A/G
6-3: 77883991G/A
6-3: 77925801T/C
6-3: 77926105A/T
6-3: 77926138C/T
6-3: 78139459T/C
6-3: 78584636A/G
6-3: 78619573T/C
6-3: 79093498C/T
6-3: 79239220A/G
6-3: 79248464G/T
6-3: 79274037G/A
6-3: 79286283C/T
6-3: 79297769C/T
6-3: 79314199T/A
6-3: 79325391A/T
6-3: 79433143C/T
6-3: 79436183C/A
6-3: 79437656C/T
6-3: 79438234A/T
6-3: 79473855G/A
6-3: 79515712A/C
6-3: 79526526A/C
6-3: 79554493T/C
6-3: 79652447A/G
6-3: 79705634C/T
6-3: 79729341T/C
6-3: 79740941G/A
6-3: 79757950A/T
6-3: 79806992G/A
6-3: 79830004C/T
6-3: 79883854A/T
6-3: 79890591C/T
6-3: 79970544T/C
6-3: 79971043C/T
6-3: 79971520T/A
6-3: 79971559T/G
6-3: 79995560G/A
6-3: 80004734C/T
6-3: 80023680A/C
6-3: 80093940C/T
6-3: 80125807T/C
6-3: 80231491A/C
6-3: 80274977C/T
6-3: 80301075T/C
6-3: 80321654T/A
6-3: 80396695T/C
6-3: 80463386C/T
6-3: 80516528A/C
6-3: 80587324T/A
6-3: 80621115G/A
6-3: 80646869T/A
6-3: 80706904G/C
6-3: 80757879T/C
6-3: 80787492G/A
6-3: 80789040T/C
6-3: 80849923C/T
6-3: 80934442C/T
6-3: 80995970C/A
6-3: 81000451C/G
6-3: 81000533A/G
6-3: 81022059G/A
6-3: 81052409C/A
6-3: 81066337C/T
6-3: 81067021T/A
6-3: 81067102G/A
6-3: 81080858A/C
6-3: 81108412A/G
6-3: 81120675C/T
6-3: 81131399A/G
6-3: 81131488C/A
6-3: 81131523T/C
6-3: 81137718G/A
6-3: 81176470A/C
6-3: 81262770T/C
6-3: 81288697G/A
6-3: 81311184A/.
6-3: 81317157G/T
6-3: 81372019A/G
6-3: 81391309C/T
6-3: 81426169A/G
6-3: 81485803C/T
6-3: 81532457G/A
6-3: 81560588G/A
6-3: 81610012A/G
6-3: 81674669T/C
6-3: 81685292A/G
6-3: 81712709A/C
6-3: 81723950T/A
6-3: 81776204G/C
6-3: 81794854C/T
6-3: 81841107G/T
6-3: 81866709G/A
6-3: 82023093C/T
6-3: 82105053C/T
6-3: 82114896C/T
6-3: 82133939A/G
6-3: 82203374G/A
6-3: 82219625G/T
6-3: 82238987C/G
6-3: 82265333T/A
6-3: 82304112G/A
6-3: 82335045T/C
6-3: 82337503T/C
6-3: 82393269T/G
6-3: 82398520G/T
6-3: 82422007C/A
6-3: 82463591A/G
6-3: 82494493A/G
6-3: 82526267T/C
6-3: 82544937T/G
6-3: 82550160G/A
6-3: 82619964C/G
6-3: 82654283C/A
6-3: 82696408C/T
6-3: 82781799C/T
6-3: 82881318T/C
6-3: 82929523G/A
6-3: 82991638G/T
6-3: 83043943A/T
6-3: 83086837T/A
6-3: 83100562A/G
6-3: 83116802T/C
6-3: 83116888T/G
6-3: 83117078T/C
6-3: 83130627A/T
6-3: 83147245C/T
6-3: 83168358A/G
6-3: 83190060T/C
6-3: 83192282G/A
6-3: 83200980T/C
6-3: 83201176T/C
6-3: 83201232C/A
6-3: 83201284C/A
6-3: 83201734A/G
6-3: 83203547C/T
6-3: 83205114T/C
6-3: 83205308T/C
6-3: 83206546G/A
6-3: 83208266A/C
6-3: 83209145C/T
6-3: 83209162C/G
6-3: 83209203T/C
6-3: 83210153G/A
6-3: 83210543G/C
6-3: 83210655C/T
6-3: 83210761A/C
6-3: 83211433T/A
6-3: 83211785C/T
6-3: 83222288G/A
6-3: 83245538T/C
6-3: 83249795A/C
6-3: 83264592C/A
6-3: 83326872G/A
6-3: 83354636G/A
6-3: 83406055T/C
6-3: 83437995A/T
6-3: 83496168T/C
6-3: 83507566T/C
6-3: 83532163T/C
6-3: 83543054G/A
6-3: 83559537T/C
6-3: 83645507A/T
6-3: 83651608A/G
6-3: 83656329C/T
6-3: 83676523C/A
6-3: 83683858G/A
6-3: 83757742T/A
6-3: 83790207T/A
6-3: 83832626G/A
6-3: 83880791C/T
6-3: 83906778T/G
6-3: 83906974C/G
6-3: 83922344G/C
6-3: 83951230G/T
6-3: 84096879A/C
6-3: 84214510G/C
6-3: 84242820T/C
6-3: 84258519A/C
6-3: 84287297T/A
6-3: 84362930C/T
6-3: 84418969G/A
6-3: 84453331C/A
6-3: 84505125C/T
6-3: 84533191G/A
6-3: 84629175A/G
6-3: 84641070C/T
6-3: 84737456G/T
6-3: 84798689T/C
6-3: 84848391T/G
6-3: 84900272A/G
6-3: 84935759G/C
6-3: 84977757C/T
6-3: 84996984C/T
6-3: 85047223G/A
6-3: 85080430C/T
6-3: 85138709A/G
6-3: 85176720A/G
6-3: 85213600C/T
6-3: 85223979A/T
6-3: 85242981G/A
6-3: 85266704C/T
6-3: 85300989A/C
6-3: 85369347G/T
6-3: 85389840C/T
6-3: 85497504T/C
6-3: 85540645T/C
6-3: 85544353T/C
6-3: 85557617G/T
6-3: 85566680C/T
6-3: 85568295T/C
6-3: 85572366C/T
6-3: 85572598T/A
6-3: 85590393C/A
6-3: 85604564A/T
6-3: 85610574T/C
6-3: 85704084G/C
6-3: 85704188T/G
6-3: 85708188C/T
6-3: 85708190C/T
6-3: 85733261C/A
6-3: 85770585A/G
6-3: 85801545A/T
6-3: 85897675C/T
6-3: 85968992G/A
6-3: 86010866G/A
6-3: 86038689T/G
6-3: 86111230G/T
6-3: 86207814A/T
6-3: 86261117T/C
6-3: 86304515G/T
6-3: 86351778G/A
6-3: 86387704A/G
6-3: 86443607T/A
6-3: 86466026G/A
6-3: 86620929C/T
6-3: 86650048A/C
6-3: 86841018G/C
6-3: 86853742A/T
6-3: 86867477T/C
6-3: 86905800G/T
6-3: 86934160A/G
6-3: 86976892A/G
6-3: 87003356C/A
6-3: 87071505T/A
6-3: 87089355C/T
6-3: 87180468C/T
6-3: 87200231C/T
6-3: 87241733A/G
6-3: 87285198A/T
6-3: 87389306G/A
6-3: 87470696T/C
6-3: 87625173A/G
6-3: 87666185C/T
6-3: 87698448C/T
6-3: 87711297G/A
6-3: 87728015T/A
6-3: 87746656A/G
6-3: 87773978G/A
6-3: 87824043G/T
6-3: 87845281A/G
6-3: 87933866T/C
6-3: 88045691G/A
6-3: 88123852C/T
6-3: 88124076G/A
6-3: 88124185G/A
6-3: 88124286A/G
6-3: 88145860C/T
6-3: 88215830T/A
6-3: 88245410G/C
6-3: 88304086A/T
6-3: 88421834C/T
6-3: 88464093G/A
6-3: 88529188C/A
6-3: 88578516C/T
6-3: 88612913G/A
6-3: 88687785A/G
6-3: 88729509A/G
6-3: 88817952T/C
6-3: 88820466C/T
6-3: 88922242T/C
6-3: 88987648C/T
6-3: 89030266T/A
6-3: 89054347A/C
6-3: 89083508C/A
6-3: 89140027T/C
6-3: 89201165T/G
6-3: 89236523G/C
6-3: 89265565C/G
6-3: 89279667G/A
6-3: 89349280T/A
6-3: 89505922T/C
6-3: 89642204G/C
6-3: 89794557G/A
6-3: 89866528A/C
6-3: 89954286G/T
6-3: 89964798T/C
6-3: 89979904A/G
6-3: 90067010G/A
6-3: 90240084T/C
6-3: 90255944A/T
6-3: 90293000A/T
6-3: 90333969A/G
6-3: 90362662A/G
6-3: 90377118T/C
6-3: 90393146T/A
6-3: 90400468A/G
6-3: 90400825A/T
6-3: 90403778T/C
6-3: 90445053A/T
6-3: 90456658C/T
6-3: 90476458A/C
6-3: 90477428A/C
6-3: 90478549G/A
6-3: 90483181G/A
6-3: 90483522G/T
6-3: 90483630C/G
6-3: 90484957A/G
6-3: 90485972T/G
6-3: 90486984A/C
6-3: 90502073T/C
6-3: 90514101C/A
6-3: 90514971A/G
6-3: 90515111G/A
6-3: 90515679A/T
6-3: 90515691C/G
6-3: 90519493G/A
6-3: 90519527T/A
6-3: 90524393C/G
6-3: 90527062A/G
6-3: 90527125A/C
6-3: 90527193A/T
6-3: 90541381C/A
6-3: 90553332T/C
6-3: 90566819T/A
6-3: 90604576A/T
6-3: 90666222A/T
6-3: 90690069T/C
6-3: 90692307A/T
6-3: 90694775G/A
6-3: 90694954T/C
6-3: 90695845A/G
6-3: 90698617T/G
6-3: 90700449T/A
6-3: 90701921A/G
6-3: 90703879T/G
6-3: 90710832C/A
6-3: 90736076T/A
6-3: 90736315A/C
6-3: 90739001A/T
6-3: 90752807T/C
6-3: 90760321C/T
6-3: 90760965T/C
6-3: 90760983C/T
6-3: 90825104C/T
6-3: 90896006A/G
6-3: 90922883G/A
6-3: 90935708T/C
6-3: 91025726A/G
6-3: 91065362T/A
6-3: 91082829C/G
6-3: 91156249G/T
6-3: 91256569T/A
6-3: 91278012T/A
6-3: 91295377A/C
6-3: 91346591A/G
6-3: 91348158T/G
6-3: 91367084G/C
6-3: 91394860G/A
6-3: 91395034A/G
6-3: 91423192A/T
6-3: 91526272G/A
6-3: 91539750T/A
6-3: 91599155G/A
6-3: 91662313T/A
6-3: 91738560C/T
6-3: 91753537A/C
6-3: 91874356T/C
6-3: 91900630G/A
6-3: 91918011T/C
6-3: 91949138T/A
6-3: 91950015A/C
6-3: 92093599C/T
6-3: 92126312A/G
6-3: 92136539C/T
6-3: 92161579T/A
6-3: 92175440C/G
6-3: 92213678A/T
6-3: 92237446G/T
6-3: 92248100T/C
6-3: 92338036T/C
6-3: 92391059G/C
6-3: 92623056A/T
6-3: 92725106A/G
6-3: 92743832T/C
6-3: 92845701C/T
6-3: 92873118C/T
6-3: 92888130C/G
6-3: 92906842T/A
6-3: 92940830A/T
6-3: 92983188C/T
6-3: 93020551G/T
6-3: 93106714A/T
6-3: 93122846A/G
6-3: 93168770T/A
6-3: 93258862T/C
6-3: 93311505T/C
6-3: 93343448G/A
6-3: 93376727C/T
6-3: 93473853A/T
6-3: 93514480C/T
6-3: 93622460G/T
6-3: 93627923C/T
6-3: 93628024A/T
6-3: 93628088C/T
6-3: 93628456A/G
6-3: 93629535A/C
6-3: 93633049T/A
6-3: 93642323T/A
6-3: 93642332A/T
6-3: 93642344G/A
6-3: 93665200T/G
6-3: 93740482C/T
6-3: 93846009T/A
6-3: 93849507C/A
6-3: 93871991C/T
6-3: 93877851A/G
6-3: 93882195A/T
6-3: 93923758T/G
6-3: 93967733C/T
6-3: 93968616G/A
6-3: 93980171G/A
6-3: 94135605G/.
6-3: 94145309T/A
6-3: 94296825T/A
6-3: 94318422A/T
6-3: 94320284A/T
6-3: 94361171C/T
6-3: 94388628A/G
6-3: 94466906C/T
6-3: 94567240G/A
6-3: 94567648C/T
6-3: 94568025T/G
6-3: 94568230C/G
6-3: 94576133G/A
6-3: 94599409G/C
6-3: 94610002T/C
6-3: 94705220C/T
6-3: 94717459A/C
6-3: 94747072T/C
6-3: 94826379C/T
6-3: 94918805C/A
6-3: 94955328C/A
6-3: 95007183T/C
6-3: 95033938T/A
6-3: 95083247T/C
6-3: 95122106G/C
6-3: 95132675T/C
6-3: 95151405T/C
6-3: 95151647C/G
6-3: 95172011G/A
6-3: 95182119T/G
6-3: 95195473C/T
6-3: 95258927C/T
6-3: 95270958G/A
6-3: 95274690A/G
6-3: 95323232T/A
6-3: 95366075A/T
6-3: 95412184T/C
6-3: 95508229G/A
6-3: 95534429G/C
6-3: 95576784A/G
6-3: 95605505C/T
6-3: 95630598T/C
6-3: 95755264A/G
6-3: 95791929G/C
6-3: 95820477A/G
6-3: 95962960C/T
6-3: 96006466A/T
6-3: 96042186A/G
6-3: 96313818G/A
6-3: 96325629A/T
6-3: 96330623T/A
6-3: 96359399G/T
6-3: 96405431T/G
6-3: 96458882A/T
6-3: 96488862G/A
6-3: 96528494C/T
6-3: 96616570T/C
6-3: 96762497A/T
6-3: 96775659G/A
6-3: 96835898A/G
6-3: 96837218C/A
6-3: 96837791A/T
6-3: 96838785G/A
6-3: 96851485A/C
6-3: 96909808G/A
6-3: 96985908G/A
6-3: 97295651G/A
6-3: 97314616A/C
6-3: 97328985C/T
6-3: 97356373T/C
6-3: 97362186G/A
6-3: 97367669T/C
6-3: 97380897T/C
6-3: 97746161A/G
6-3: 97774058A/G
6-3: 97782117G/T
6-3: 97788199A/G
6-3: 97801149T/C
6-3: 98037814A/G
6-3: 98145106T/A
6-3: 98190490A/G
6-3: 98296822A/C
6-3: 98334683T/C
6-3: 98412979A/C
6-3: 98542731T/A
6-3: 98543200A/G
6-3: 98543223G/T
6-3: 98543258G/A
6-3: 98543285C/A
6-3: 98543297C/T
6-3: 98543308C/T
6-3: 98744636C/T
6-3: 98854712T/A
6-3: 99340472C/T
6-3: 99482244T/G
6-3: 99851660G/T
6-3: 99852750A/G
6-3: 99852901T/A
6-3: 99853292A/G
6-3: 99854256C/T
6-3: 99863581T/A
6-4: 68839G/A
6-4: 121215T/G
6-4: 450961G/A
6-4: 456963G/A
6-4: 482224C/G
6-4: 567613A/G
6-4: 621450T/G
6-4: 631484A/T
6-4: 773904C/T
6-4: 832923G/A
6-4: 880590A/T
6-4: 1022395T/A
6-4: 1023534T/A
6-4: 1023706C/T
6-4: 1030045A/T
6-4: 1102738A/C
6-4: 1173832G/A
6-4: 1233517T/A
6-4: 1314640C/A
6-4: 1335120C/G
6-4: 1418033G/A
6-4: 1432768A/T
6-4: 1452942T/A
6-4: 1478334A/G
6-4: 1529210T/G
6-4: 1563891T/A
6-4: 1595524C/T
6-4: 1672238C/A
6-4: 1689861T/A
6-4: 1700273A/G
6-4: 1728428C/T
6-4: 1729534A/G
6-4: 1739344T/A
6-4: 1778476A/T
6-4: 1877395A/T
6-4: 1920091T/C
6-4: 1988742A/G
6-4: 2022394G/T
6-4: 2098757T/A
6-4: 2184768A/G
6-4: 2229128C/T
6-4: 2277400G/A
6-4: 2296840A/G
6-4: 2393542G/C
6-4: 2427156T/C
6-4: 2470097T/A
6-4: 2470118C/A
6-4: 2484652G/A
6-4: 2539367T/G
6-4: 2637019C/A
6-4: 2686137A/G
6-4: 2744730A/G
6-4: 2744733T/C
6-4: 2814012A/G
6-4: 2883683T/A
6-4: 2897964G/A
6-4: 2986815T/A
6-4: 3005587G/A
6-4: 3042550C/T
6-4: 3044123T/C
6-4: 3059274T/C
6-4: 3081620A/G
6-4: 3129060G/A
6-4: 3168558A/G
6-4: 3202928C/A
6-4: 3267084C/T
6-4: 3359052T/C
6-4: 3384944A/G
6-4: 3403250C/T
6-4: 3414705C/T
6-4: 3457187C/T
6-4: 3466893A/G
6-4: 3466914C/T
6-4: 3467364T/C
6-4: 3467465C/T
6-4: 3467703G/T
6-4: 3516640A/T
6-4: 3527753G/T
6-4: 3571667T/A
6-4: 3625746A/G
6-4: 3632133T/A
6-4: 3632464A/C
6-4: 3632469T/A
6-4: 3643495T/C
6-4: 3664365A/G
6-4: 3689263A/T
6-4: 3732487T/C
6-4: 3747525C/T
6-4: 3805945G/C
6-4: 3808281G/A
6-4: 3808776G/A
6-4: 3810189A/G
6-4: 3811655A/G
6-4: 3811856G/A
6-4: 3811915G/A
6-4: 3821842C/G
6-4: 3827547T/A
6-4: 3851545G/A
6-4: 3874019T/A
6-4: 3882933A/T
6-4: 3883822A/C
6-4: 3883864G/T
6-4: 3885918T/G
6-4: 3893124C/T
6-4: 3894537G/A
6-4: 3894695G/T
6-4: 3894956A/G
6-4: 3895094C/A
6-4: 3907120T/A
6-4: 3923270C/A
6-4: 3937506G/C
6-4: 3951938G/A
6-4: 3987454G/A
6-4: 4008664G/A
6-4: 4019863A/G
6-4: 4031515T/C
6-4: 4038126G/A
6-4: 4041582T/C
6-4: 4058151T/C
6-4: 4061510A/G
6-4: 4062458G/A
6-4: 4076177G/A
6-4: 4106297C/T
6-4: 4107635T/A
6-4: 4107770T/C
6-4: 4114395C/A
6-4: 4114642C/A
6-4: 4114830G/A
6-4: 4121641C/A
6-4: 4123238C/T
6-4: 4136085C/A
6-4: 4148423G/A
6-4: 4159732A/C
6-4: 4160456C/G
6-4: 4160592G/A
6-4: 4160739C/T
6-4: 4172781T/C
6-4: 4173629A/T
6-4: 4173835G/C
6-4: 4173912T/G
6-4: 4175207T/C
6-4: 4175311A/G
6-4: 4184405G/A
6-4: 4202441A/G
6-4: 4227568A/G
6-4: 4253777C/A
6-4: 4268341A/C
6-4: 4293118A/G
6-4: 4304078G/A
6-4: 4408032C/T
6-4: 4408465T/A
6-4: 4408567A/T
6-4: 4434891T/C
6-4: 4449246G/A
6-4: 4463806G/C
6-4: 4530785G/A
6-4: 4541615T/C
6-4: 4541812C/T
6-4: 4550106T/C
6-4: 4623347C/A
6-4: 4644760C/T
6-4: 4647902A/T
6-4: 4663525T/C
6-4: 4695213C/T
6-4: 4759961G/A
6-4: 4821451A/T
6-4: 4878662C/G
6-4: 4922726A/C
6-4: 4934624T/A
6-4: 4954196T/G
6-4: 5008188A/T
6-4: 5013617T/C
6-4: 5017297A/G
6-4: 5018908T/A
6-4: 5041475A/G
6-4: 5066801G/A
6-4: 5066900G/A
6-4: 5070158T/C
6-4: 5078147T/C
6-4: 5078809C/T
6-4: 5085386C/A
6-4: 5085472C/A
6-4: 5085710G/A
6-4: 5086577C/A
6-4: 5086644A/T
6-4: 5087095T/C
6-4: 5087104G/A
6-4: 5087198A/G
6-4: 5087388T/A
6-4: 5087494G/A
6-4: 5087960C/T
6-4: 5088065A/G
6-4: 5088116T/C
6-4: 5088160T/C
6-4: 5088508A/T
6-4: 5088774C/T
6-4: 5088819C/A
6-4: 5089192G/A
6-4: 5089329C/T
6-4: 5089436C/T
6-4: 5100821A/G
6-4: 5127232A/T
6-4: 5181106A/G
6-4: 5217176C/A
6-4: 5252908T/C
6-4: 5309265C/T
6-4: 5344774A/G
6-4: 5369218G/A
6-4: 5407965G/A
6-4: 5443168G/T
6-4: 5487079C/T
6-4: 5499392T/G
6-4: 5611086G/A
6-4: 5631574T/C
6-4: 5647785A/G
6-4: 5669178T/C
6-4: 5694512A/T
6-4: 5697929T/A
6-4: 5741548C/T
6-4: 5780485A/G
6-4: 5859367A/G
6-4: 5873693G/A
6-4: 5873935T/G
6-4: 5874103A/G
6-4: 5887600T/C
6-4: 5897849A/G
6-4: 5914145A/T
6-4: 5932421A/G
6-4: 5961282T/A
64: 5964027T/G
6-4: 5964045G/A
6-4: 6027450T/C
6-4: 6076124G/A
6-4: 6126980A/C
6-4: 6197667T/G
6-4: 6226839G/T
6-4: 6293277G/A
6-4: 6315439T/C
6-4: 6346743T/C
6-4: 6363721A/G
6-4: 6415138C/T
6-4: 6453131G/A
6-4: 6492792T/C
6-4: 6525373T/G
6-4: 6525893G/T
6-4: 6536506G/T
6-4: 6539483A/T
6-4: 6550474C/A
6-4: 6561143T/A
6-4: 6579728G/A
6-4: 6612579G/A
6-4: 6625408G/A
6-4: 6628140G/A
6-4: 6639123A/G
6-4: 6679365G/A
6-4: 6681282C/T
6-4: 6682310C/T
6-4: 6682471A/T
6-4: 6682589T/C
6-4: 6682994C/T
6-4: 6688930G/T
6-4: 6689048G/C
6-4: 6689197A/G
6-4: 6689311G/A
6-4: 6689397C/T
6-4: 6689437G/A
6-4: 6690482G/A
6-4: 6690839G/A
6-4: 6698958A/G
6-4: 6717635A/G
6-4: 6825878T/A
6-4: 6877285G/A
6-4: 6882903A/G
6-4: 6888180A/G
6-4: 6918213C/G
6-4: 6919409A/G
6-4: 6919767A/G
6-4: 6919863A/G
6-4: 6920872C/T
6-4: 6921220C/T
6-4: 6929855G/A
6-4: 6965248T/C
6-4: 6991053T/C
6-4: 7041763A/T
6-4: 7114554G/T
6-4: 7134794C/T
6-4: 7136771G/A
6-4: 7141200G/A
6-4: 7202301T/C
6-4: 7235896T/A
6-4: 7256350C/A
6-4: 7287637G/A
6-4: 7319815C/A
6-4: 7356833C/T
6-4: 7386009G/A
6-4: 7397738G/A
6-4: 7410656T/A
6-4: 7416328G/A
6-4: 7420592G/C
6-4: 7420822C/A
6-4: 7421140C/T
6-4: 7421156A/C
6-4: 7421157A/G
6-4: 7421432A/T
6-4: 7422016C/A
6-4: 7458230T/C
6-4: 7519829T/C
6-4: 7525610T/A
6-4: 7564814A/G
6-4: 7585241G/A
6-4: 7612716G/A
6-4: 7653888G/T
6-4: 7699125A/T
6-4: 7715165C/A
6-4: 7725855C/T
6-4: 7792003A/G
6-4: 7821264T/C
6-4: 7834285A/G
6-4: 7860693A/T
6-4: 7876960G/A
6-4: 7910358T/C
6-4: 7959586T/C
6-4: 7990143A/G
6-4: 8023488C/T
6-4: 8086799C/T
6-4: 8094517T/A
6-4: 8094894G/A
6-4: 8095197T/G
6-4: 8097390A/C
6-4: 8109468T/C
6-4: 8119805G/T
6-4: 8157744T/A
6-4: 8184500T/C
6-4: 8184690T/C
6-4: 8184818C/T
6-4: 8184949C/T
6-4: 8184992G/T
6-4: 8185073C/T
6-4: 8207475T/A
6-4: 8232067T/A
6-4: 8298840A/T
6-4: 8309298C/T
6-4: 8321736T/C
6-4: 8358849G/A
6-4: 8458844A/G
6-4: 8463052C/T
6-4: 8473317G/C
6-4: 8474246T/C
6-4: 8474812A/G
6-4: 8474821A/C
6-4: 8574460C/A
6-4: 8643077C/T
6-4: 8664437A/G
6-4: 8699461T/C
6-4: 8711057G/A
6-4: 8726806G/A
6-4: 8838416G/C
6-4: 8873161C/A
6-4: 8931583C/T
6-4: 8956280T/C
6-4: 8991076C/A
6-4: 8992950T/C
6-4: 9027379C/A
6-4: 9064430G/A
6-4: 9081568G/T
6-4: 9102408G/A
6-4: 9114339T/C
6-4: 9221382G/A
6-4: 9256476G/A
6-4: 9291463C/T
6-4: 9311880G/A
6-4: 9321144C/T
6-4: 9322020A/G
6-4: 9322897T/C
6-4: 9325087T/C
6-4: 9325209C/A
6-4: 9353951G/C
6-4: 9368476A/G
6-4: 9381284A/G
6-4: 9381938T/C
6-4: 9396010G/A
6-4: 9416650G/A
6-4: 9418144T/G
6-4: 9473630G/A
6-4: 9525420C/T
6-4: 9640688A/G
6-4: 9663018A/C
6-4: 9688304G/A
6-4: 9720405T/C
6-4: 9781346G/A
6-4: 9811348T/C
6-4: 9876918G/A
6-4: 9915425C/T
6-4: 9983063A/G
6-4: 9997823T/C
6-4: 10010062G/C
6-4: 10050001T/A
6-4: 10077519C/A
6-4: 10088195C/A
6-4: 10089273C/T
6-4: 10091910A/C
6-4: 10092578T/A
6-4: 10093665A/G
6-4: 10093925A/C
6-4: 10159624A/G
6-4: 10170690G/A
6-4: 10192003G/A
6-4: 10193384G/T
6-4: 10206106C/T
6-4: 10246991C/A
6-4: 10274025T/C
6-4: 10288648G/A
6-4: 10288889G/A
6-4: 10289312A/T
6-4: 10290281A/G
6-4: 10290820G/C
6-4: 10298757A/T
6-4: 10299199A/G
6-4: 10299304G/A
6-4: 10323171G/T
6-4: 10336562C/A
6-4: 10350503T/C
6-4: 10376849T/C
6-4: 10405377T/C
6-4: 10463100A/T
6-4: 10488351A/C
6-4: 10523088C/T
6-4: 10562303A/G
6-4: 10595291T/A
6-4: 10624367C/T
6-4: 10636788C/A
6-4: 10764288A/G
6-4: 10770568T/A
6-4: 10770667T/A
6-4: 10822404C/T
6-4: 10848792G/.
6-4: 10877339G/T
6-4: 10877552G/A
6-4: 10877555G/A
6-4: 10897075A/T
6-4: 10923555C/T
6-4: 10990744G/A
6-4: 11001008G/A
6-4: 11013466C/T
6-4: 11024497A/G
6-4: 11034018G/A
6-4: 11034136G/A
6-4: 11034323C/T
6-4: 11034358T/C
6-4: 11034428T/A
6-4: 11040801C/A
6-4: 11079534C/A
6-4: 11099961C/A
6-4: 11102357G/A
6-4: 11103135A/C
6-4: 11103242T/C
6-4: 11103676C/T
6-4: 11103790C/T
6-4: 11103999G/A
6-4: 11104219G/C
6-4: 11104823A/T
6-4: 11105183C/T
6-4: 11108183C/T
6-4: 11108384G/T
6-4: 11108650A/G
6-4: 11109308C/T
6-4: 11109438A/G
6-4: 11109674T/C
6-4: 11109852G/A
6-4: 11110217T/C
6-4: 11181183T/C
6-4: 11197412G/A
6-4: 11230561C/T
6-4: 11287131A/C
6-4: 11316819A/T
6-4: 11316857C/T
6-4: 11382177G/A
6-4: 11384443T/A
6-4: 11398407C/T
6-4: 11424107C/G
6-4: 11436769C/T
6-4: 11463884T/G
6-4: 11522545A/G
6-4: 11534343A/G
6-4: 11550478A/T
6-4: 11628658C/T
6-4: 11692510A/T
6-4: 11704088A/T
6-4: 11749947G/A
6-4: 11810417C/A
6-4: 11874889G/A
6-4: 11889158A/G
6-4: 11929434A/G
6-4: 11959681A/G
6-4: 11970841T/A
6-4: 11970886A/G
6-4: 11972600C/A
6-4: 11974591C/T
6-4: 11974722T/C
6-4: 11974824C/T
6-4: 11975395G/T
6-4: 11975528C/T
6-4: 11976035T/A
6-4: 11976147A/G
6-4: 11976203G/A
6-4: 11976378G/T
6-4: 11978278T/G
6-4: 11978292C/T
6-4: 11984518A/G
6-4: 11995352C/A
6-4: 12001957T/C
6-4: 12004718A/C
6-4: 12037524G/C
6-4: 12042593T/A
6-4: 12046028G/A
6-4: 12046197C/T
6-4: 12048829T/A
6-4: 12071889T/C
6-4: 12082374C/T
6-4: 12085336G/C
6-4: 12085439G/T
6-4: 12099702C/T
6-4: 12135192G/A
6-4: 12183305A/G
6-4: 12227149G/A
6-4: 12274372C/T
6-4: 12285136C/T
6-4: 12285431A/C
6-4: 12287041T/C
6-4: 12349393T/G
6-4: 12487897T/G
6-4: 12511139T/C
6-4: 12658752T/A
6-4: 12705651A/G
6-4: 12769872C/A
6-4: 12864272G/A
6-4: 12885067A/C
6-4: 12906279T/C
6-4: 12919503A/T
6-4: 12940875A/G
6-4: 12994699G/A
6-4: 12995124G/T
6-4: 13025749C/T
6-4: 13028840A/C
6-4: 13077649C/T
6-4: 13088406T/G
6-4: 13136146G/A
6-4: 13179586C/T
6-4: 13211077A/G
6-4: 13251420T/C
6-4: 13265143C/A
6-4: 13291419C/T
6-4: 13320015G/A
6-4: 13344016G/A
6-4: 13363427G/C
6-4: 13423437C/A
6-4: 13476571A/T
6-4: 13492675C/G
6-4: 13659148G/A
6-4: 13727735G/A
6-4: 13749009C/T
6-4: 13789120A/G
6-4: 13820767C/A
6-4: 13964307C/T
6-4: 13965047C/T
6-4: 13965103A/C
6-4: 13975006T/C
6-4: 14010747A/C
6-4: 14105852G/T
6-4: 14110611G/C
6-4: 14126460T/C
6-4: 14166298A/T
6-4: 14194540T/C
6-4: 14245532C/T
6-4: 14245546T/C
6-4: 14245649T/C
6-4: 14246101A/G
6-4: 14246568C/T
6-4: 14246582C/A
6-4: 14246605G/A
6-4: 14248934T/G
6-4: 14249394T/G
6-4: 14249451A/G
6-4: 14249839A/G
6-4: 14250050C/G
6-4: 14250156A/C
6-4: 14250295C/T
6-4: 14256719T/C
6-4: 14267021C/T
6-4: 14430667A/G
6-4: 14433258A/G
6-4: 14444646G/A
6-4: 14478066C/T
6-4: 14488351G/A
6-4: 14489355C/A
6-4: 14513739G/A
6-4: 14518870G/A
6-4: 14522635T/G
6-4: 14528334T/A
6-4: 14529111A/G
6-4: 14529775A/G
6-4: 14540993T/C
6-4: 14553573C/T
6-4: 14555270G/A
6-4: 14557133T/C
6-4: 14557495C/T
6-4: 14580451T/C
6-4: 14636922G/A
6-4: 14800474A/G
6-4: 14800489T/C
6-4: 14800504G/A
6-4: 14800859T/C
6-4: 14806733G/A
6-4: 14819062A/C
6-4: 14829104G/C
6-4: 14847334A/T
6-4: 14869114G/C
6-4: 14883523G/A
6-4: 14900941C/T
6-4: 14901506G/A
6-4: 14901645T/C
6-4: 14902953A/G
6-4: 14903816G/A
6-4: 14923353C/A
6-4: 14933608A/G
6-4: 14953179T/C
6-4: 14967282A/G
6-4: 14985137T/C
6-4: 15024324A/T
6-4: 15039975C/T
6-4: 15056057A/G
6-4: 15068927G/A
6-4: 15082819T/A
6-4: 15115182T/A
6-4: 15140773T/C
6-4: 15173633G/A
6-4: 15174621C/G
6-4: 15174886A/G
6-4: 15174992T/C
6-4: 15187273C/A
6-4: 15196687G/A
6-4: 15199608T/C
6-4: 15199717A/G
6-4: 15202380T/A
6-4: 15225245G/A
6-4: 15263234A/T
6-4: 15273855G/A
6-4: 15305741C/T
6-4: 15319594A/T
6-4: 15362951T/A
6-4: 15366319C/T
6-4: 15367953G/A
6-4: 15368024C/T
6-4: 15368072G/T
6-4: 15368181C/T
6-4: 15368492T/C
6-4: 15369855T/G
6-4: 15374791C/T
6-4: 15375968T/C
6-4: 15383976G/A
6-4: 15387213C/T
6-4: 15482102G/T
6-4: 15530375A/C
6-4: 15542316C/A
6-4: 15552890T/C
6-4: 15565484A/G
6-4: 15594323C/T
6-4: 15600227G/T
6-4: 15644372T/A
6-4: 15694786T/G
6-4: 15766682A/C
6-4: 15841978C/T
6-4: 15846014C/T
6-4: 15850982G/C
6-4: 15852018T/A
6-4: 15852223G/A
6-4: 15854499T/A
6-4: 15872204A/C
6-4: 15891838C/T
6-4: 15909272T/A
6-4: 15930058T/C
6-4: 15955254A/C
6-4: 15981349C/T
6-4: 16016592T/A
6-4: 16022632T/A
6-4: 16030780G/C
6-4: 16052414C/T
6-4: 16068015A/T
6-4: 16078535T/G
6-4: 16098837T/G
6-4: 16110440A/G
6-4: 16120932T/A
6-4: 16150921A/T
6-4: 16155820G/A
6-4: 16178175A/T
6-4: 16182108T/C
6-4: 16193586A/C
6-4: 16199167T/C
6-4: 16199274A/G
6-4: 16199388C/T
6-4: 16200805C/T
6-4: 16203777A/T
6-4: 16205157T/G
6-4: 16217495A/C
6-4: 16228067C/T
6-4: 16256612G/A
6-4: 16287650T/C
6-4: 16289464A/G
6-4: 16316893C/G
6-4: 16322359A/T
6-4: 16329781C/T
6-4: 16367028G/A
6-4: 16389164C/A
6-4: 16419744C/T
6-4: 16449251A/T
6-4: 16478555G/C
6-4: 16539549C/T
6-4: 16619225A/T
6-4: 16661793G/A
6-4: 16698848C/G
6-4: 16714902C/A
6-4: 16714958T/C
6-4: 16717805C/T
6-4: 16718126A/G
6-4: 16718254A/C
6-4: 16724989C/A
6-4: 16728552G/T
6-4: 16729083A/T
6-4: 16731169G/T
6-4: 16731379T/C
6-4: 16732982C/T
6-4: 16733307C/T
6-4: 16757058C/T
6-4: 16760628C/T
6-4: 16760767A/T
6-4: 16768703G/T
6-4: 16772493C/T
6-4: 16772592C/T
6-4: 16772683C/T
6-4: 16773167A/G
6-4: 16773188C/T
6-4: 16773215A/T
6-4: 16773928A/G
6-4: 16796667C/T
6-4: 16806620C/.
6-4: 16806642G/A
6-4: 16806680C/T
6-4: 16806717T/C
6-4: 16806732C/A
6-4: 16806788C/T
6-4: 16806791C/T
6-4: 16806804T/C
6-4: 16806900T/C
6-4: 16810082C/T
6-4: 16838903C/A
6-4: 16843008C/T
6-4: 16843048A/C
6-4: 16843051G/A
6-4: 16843063A/G
6-4: 16843156G/C
6-4: 16857751A/.
6-4: 16857767G/A
6-4: 16857779G/A
6-4: 16857796A/G
6-4: 16857804C/T
6-4: 16857809C/A
6-4: 16857869T/C
6-4: 16857879T/G
6-4: 16857980T/A
6-4: 16858008T/G
6-4: 16869075G/A
6-4: 16879823A/G
6-4: 16894322G/A
6-4: 16899156T/G
6-4: 16907210C/T
6-4: 16907808G/T
6-4: 16918764A/C
6-4: 16929904C/T
6-4: 16972246T/C
6-4: 16983328T/C
6-4: 16997187T/G
6-4: 17032313T/C
6-4: 17039591C/T
6-4: 17039750G/A
6-4: 17046917G/A
6-4: 17072447G/A
6-4: 17075878G/A
6-4: 17098404T/G
6-4: 17108740T/C
6-4: 17120994T/C
6-4: 17133711G/T
6-4: 17140931A/G
6-4: 17141076C/A
6-4: 17141077G/A
6-4: 17163547T/G
6-4: 17198282C/A
6-4: 17276844G/A
6-4: 17367205C/A
6-4: 17381958A/G
6-4: 17414517T/A
6-4: 17555742A/G
6-4: 17590208T/C
6-4: 17605085C/T
6-4: 17667278C/T
6-4: 17726956A/C
6-4: 17826283A/T
6-4: 17869499A/C
6-4: 17882809A/G
6-4: 17893567G/A
6-4: 17898010A/G
6-4: 17907456C/T
6-4: 17924173A/C
6-4: 17924699T/A
6-4: 17924816T/A
6-4: 17931698G/A
6-4: 17933226C/A
6-4: 17933567T/C
6-4: 17934473A/C
6-4: 17935345G/A
6-4: 17956990G/A
6-4: 17964631A/T
6-4: 17987779T/A
6-4: 18048494T/A
6-4: 18077640C/T
6-4: 18083499C/T
6-4: 18106037G/A
6-4: 18106072A/C
6-4: 18115667T/A
6-4: 18116075C/T
6-4: 18116776G/A
6-4: 18120002C/T
6-4: 18129417G/A
6-4: 18161287T/C
6-4: 18197461A/G
6-4: 18281062C/T
6-4: 18294027A/G
6-4: 18294605C/T
6-4: 18294770G/A
6-4: 18295762G/A
6-4: 18295813A/G
6-4: 18295837A/T
6-4: 18295840G/A
6-4: 18295984T/A
6-4: 18295996G/A
6-4: 18296041G/A
6-4: 18296060C/T
6-4: 18296075C/T
6-4: 18296152A/G
6-4: 18296176T/G
6-4: 18313664T/G
6-4: 18331633T/A
6-4: 18331815A/C
6-4: 18352266G/C
6-4: 18358548C/G
6-4: 18371614T/C
6-4: 18383700C/T
6-4: 18383959A/G
6-4: 18396149T/C
6-4: 18401298A/C
6-4: 18401559C/T
6-4: 18402015C/T
6-4: 18402092C/G
6-4: 18402233A/G
6-4: 18404414A/T
6-4: 18405558A/G
6-4: 18405706A/G
6-4: 18405901A/G
6-4: 18406018C/T
6-4: 18406309C/A
6-4: 18406997G/A
6-4: 18408014C/T
6-4: 18408150G/A
6-4: 18408378C/T
6-4: 18408401G/A
6-4: 18408511G/A
6-4: 18408513C/T
6-4: 18408649C/A
6-4: 18408787T/A
6-4: 18408978G/A
6-4: 18409055G/A
6-4: 18409147A/G
6-4: 18409460C/T
6-4: 18409570G/A
6-4: 18409725A/G
6-4: 18417415A/G
6-4: 18448037A/G
6-4: 18535556G/T
6-4: 18604553A/G
6-4: 18614760C/A
6-4: 18614764C/A
6-4: 18637364A/T
6-4: 18647742C/T
6-4: 18657337A/G
6-4: 18658105C/T
6-4: 18658955A/G
6-4: 18659534T/A
6-4: 18702340A/G
6-4: 18722499G/T
6-4: 18745133C/G
6-4: 18747659G/A
6-4: 18747801A/T
6-4: 18753128G/T
6-4: 18753137G/T
6-4: 18756047G/A
6-4: 18769871G/C
6-4: 18812127T/A
6-4: 18880108T/G
6-4: 18886768G/A
6-4: 18931997T/G
6-4: 18942707C/T
6-4: 18953896G/A
6-4: 18956300C/T
6-4: 18956402C/G
6-4: 18956750T/G
6-4: 18970435T/C
6-4: 18985689A/G
6-4: 19008532T/A
6-4: 19056233G/T
6-4: 19066792C/T
6-4: 19080491T/A
6-4: 19081691A/C
6-4: 19081799T/C
6-4: 19082000T/C
6-4: 19082329T/C
6-4: 19106212C/A
6-4: 19139820C/G
6-4: 19164859G/A
6-4: 19172399G/A
6-4: 19177781T/G
6-4: 19202839G/A
6-4: 19255058C/T
6-4: 19274519G/T
6-4: 19274681A/C
6-4: 19292082C/A
6-4: 19313480G/T
6-4: 19353825T/G
6-4: 19395381G/T
6-4: 19396311T/C
6-4: 19396895C/T
6-4: 19414745A/G
6-4: 19418030G/A
6-4: 19418149G/A
6-4: 19430518T/G
6-4: 19432402C/T
6-4: 19432602G/A
6-4: 19435144G/A
6-4: 19435787G/C
6-4: 19435983T/A
6-4: 19442045C/T
6-4: 19442502A/G
6-4: 19442930A/G
6-4: 19443200T/C
6-4: 19443347G/C
6-4: 19443533T/A
6-4: 19462224C/A
6-4: 19463394G/A
6-4: 19474737C/T
6-4: 19564758T/C
6-4: 19575067T/G
6-4: 19603862A/G
6-4: 19624961C/T
6-4: 19637640A/C
6-4: 19648530A/G
6-4: 19661766A/G
6-4: 19679840A/G
6-4: 19699507T/G
6-4: 19712013G/T
6-4: 19736244A/T
6-4: 19768581T/C
6-4: 19778639A/T
6-4: 19781392A/T
6-4: 19781407C/.
6-4: 19781434T/A
6-4: 19781443T/G
6-4: 19781484G/.
6-4: 19983457G/A
6-4: 20104031T/A
6-4: 20154433A/T
6-4: 20212766T/C
6-4: 20225293C/A
6-4: 20264982A/G
6-4: 20425562A/G
6-4: 20428358A/T
6-4: 20436694A/G
6-4: 20457359C/T
6-4: 20637517C/T
6-4: 20807933C/T
6-4: 20826262T/C
6-4: 20877982T/A
6-4: 20922387A/G
6-4: 20944246C/T
6-4: 21056985A/C
6-4: 21447612A/G
6-4: 21471990T/C
6-4: 21553823A/G
6-4: 21588222T/C
6-4: 21750995T/A
6-4: 21763005A/G
6-4: 21769998G/A
6-4: 21771076C/T
6-4: 21806622T/C
6-4: 21840314A/C
6-4: 21974309G/A
6-4: 22011176T/G
6-4: 22037177T/C
6-4: 22316883G/C
6-4: 22331237G/A
6-4: 22345207C/G
6-4: 22385988C/T
6-4: 22440719G/A
6-4: 23024670C/T
6-4: 23243443C/T
6-4: 23254191C/T
6-4: 23300160A/G
6-4: 24280428A/G
6-4: 25842676A/G
6-4: 26489134A/C
6-4: 26500214G/T
6-4: 26503859T/A
6-4: 26505964T/G
6-4: 26511596T/G
6-4: 26522374T/A
6-4: 26908983T/C
6-4: 27052770A/T
6-4: 27144655T/C
6-4: 27621804T/C
6-4: 27622018T/A
6-4: 27623535T/G
6-4: 27640897C/T
6-4: 27753910T/G
6-4: 27770491T/A
6-4: 27812045T/A
6-4: 27833970C/G
6-4: 27887524C/T
6-4: 27920956G/A
6-4: 27996675C/G
6-4: 28145142G/A
6-4: 28429776G/T
6-4: 28515261G/T
6-4: 28677101G/A
6-4: 28782188G/T
6-4: 28805469T/A
6-4: 28826192A/G
6-4: 29051369T/G
6-4: 29120837G/A
6-4: 29184722T/A
6-4: 29219496A/G
6-4: 29300386C/A
6-4: 29476171G/A
6-4: 29499968A/T
6-4: 29515291G/A
6-4: 29544207T/C
6-4: 29605350C/T
6-4: 29609042C/T
6-4: 29621205T/A
6-4: 29673974G/A
6-4: 29813248G/T
6-4: 29814944C/T
6-4: 29867196G/A
6-4: 29886707A/G
6-4: 29930336A/G
6-4: 29956503T/C
6-4: 29967316T/C
6-4: 29993562G/T
6-4: 30037765C/T
6-4: 30053914A/G
6-4: 30070251A/G
6-4: 30100867A/G
6-4: 30143864G/A
6-4: 30162346T/A
6-4: 30197046C/G
6-4: 30251319G/A
6-4: 30262053T/C
6-4: 30329978T/A
6-4: 30348556T/C
6-4: 30364572G/A
6-4: 30368735A/G
6-4: 30420011T/A
6-4: 30504337G/A
6-4: 30531089C/T
6-4: 30542271T/C
6-4: 30609029A/T
6-4: 30610018C/T
6-4: 30623066C/T
6-4: 30642243A/G
6-4: 30743551T/G
6-4: 30777254C/T
6-4: 30856111G/C
6-4: 30908554A/G
6-4: 31006271A/T
6-4: 31082448C/A
6-4: 31103038A/G
6-4: 31117344C/T
6-4: 31128933G/A
6-4: 31143020G/T
6-4: 31159090A/G
6-4: 31159426C/T
6-4: 31160793G/T
6-4: 31160797T/A
6-4: 31162074C/A
6-4: 31165014A/C
6-4: 31165020C/T
6-4: 31245874A/G
6-4: 31276113A/G
6-4: 31302848G/C
6-4: 31393868A/C
6-4: 31550267C/T
6-4: 31553559G/A
6-4: 31649527A/G
6-4: 31661047A/G
6-4: 31805772T/C
6-4: 31918705A/G
6-4: 31940220T/C
6-4: 32034245C/T
6-4: 32112987C/A
6-4: 32124192C/T
6-4: 32168358C/T
6-4: 32188810G/A
6-4: 32262745C/G
6-4: 32277028C/T
6-4: 32297583G/A
6-4: 32346956G/A
6-4: 32403063A/G
6-4: 32533666G/A
6-4: 32536932T/C
6-4: 32548591A/G
6-4: 32572833G/C
6-4: 32826799T/C
6-4: 32930875G/A
6-4: 33029265T/A
6-4: 33078141G/A
6-4: 33090217A/G
6-4: 33168906G/A
6-4: 33184591A/T
6-4: 33243252A/G
6-4: 33245426T/C
6-4: 33245900T/G
6-4: 33246039T/A
6-4: 33248382T/C
6-4: 33248492C/T
6-4: 33287933T/C
6-4: 33325339C/T
6-4: 33355241C/T
6-4: 33357050T/A
6-4: 33389478A/G
6-4: 33429989A/C
6-4: 33468434A/T
6-4: 33486437T/A
6-4: 33570948T/C
6-4: 33583782A/C
6-4: 33631329G/A
6-4: 33659062C/T
6-4: 33678894G/C
6-4: 33815128G/A
6-4: 33830019C/T
6-4: 33860062A/C
6-4: 33891863T/C
6-4: 33980934T/C
6-4: 33991009G/A
6-4: 34019123C/A
6-4: 34029308T/C
6-4: 34031800T/A
6-4: 34034793C/T
6-4: 34078557G/A
6-4: 34079063C/T
6-4: 34104683C/A
6-4: 34221474G/A
6-4: 34235757C/A
6-4: 34249134C/T
6-4: 34266914A/T
6-4: 34325197C/A
6-4: 34342778T/A
6-4: 34367906A/C
6-4: 34528878A/G
6-4: 34530183G/A
6-4: 34530841C/T
6-4: 34578975C/T
6-4: 34590374C/A
6-4: 34729677C/T
6-4: 34919086C/T
6-4: 35021528G/A
6-4: 35022251T/G
6-4: 35022254A/G
6-4: 35196156A/G
6-4: 35220175A/G
6-4: 35230897T/A
6-4: 35292984T/G
6-4: 35316616G/A
6-4: 35317117C/A
6-4: 35337887C/T
6-4: 35337929A/G
6-4: 35369444T/G
6-4: 35394621G/A
6-4: 35441592C/A
6-4: 35492078A/G
6-4: 35547980C/A
6-4: 35736166T/C
6-4: 35861470A/T
6-4: 35897102A/C
6-4: 35971314C/T
6-4: 36035027G/A
6-4: 36036979T/G
6-4: 36128263A/G
6-4: 36222403C/T
6-4: 36248965T/C
6-4: 36272291C/A
6-4: 36295093C/T
6-4: 36386506C/T
6-4: 36398116G/A
6-4: 36399894G/A
6-4: 36427722T/C
6-4: 36508750C/G
6-4: 36541589A/G
6-4: 36568803C/T
6-4: 36607098A/G
6-4: 36617655C/A
6-4: 36770939A/G
6-4: 36776344C/G
6-4: 36776356T/C
6-4: 36781418T/C
6-4: 36783545T/C
6-4: 36798745A/T
6-4: 36840429T/C
6-4: 36865801C/A
6-4: 36892922C/T
6-4: 36967238G/A
6-4: 36971513T/A
6-4: 37081819C/T
6-4: 37102033G/C
6-4: 37164610T/C
6-4: 37237139A/T
6-4: 37248938A/T
6-4: 37326023G/A
6-4: 37367690G/A
6-4: 37475380A/G
6-4: 37494116T/A
6-4: 37515141T/C
6-4: 37570379C/A
6-4: 37630523T/C
6-4: 37671388C/T
6-4: 37711130A/T
6-4: 37748938C/A
6-4: 37790447A/G
6-4: 37793792T/C
6-4: 37822408T/C
6-4: 37870854G/A
6-4: 37910925C/T
6-4: 37937836T/A
6-4: 37983380G/A
6-4: 38031246A/G
6-4: 38061197A/G
6-4: 38105131T/C
6-4: 38171043C/T
6-4: 38223967G/C
6-4: 38286611C/T
6-4: 38321328G/A
6-4: 38346554T/C
6-4: 38402901A/G
6-4: 38426247G/A
6-4: 38675973G/T
6-4: 38703600G/A
6-4: 38744969C/T
6-4: 38773603A/G
6-4: 38818559A/T
6-4: 38820849A/G
6-4: 38832412T/C
6-4: 38841911A/G
6-4: 38878434G/A
6-4: 38932115G/A
6-4: 39039941G/A
6-4: 39267167C/T
6-4: 39382349T/C
6-4: 39526094C/T
6-4: 39538150A/G
6-4: 39542135C/T
6-4: 39555399T/C
6-4: 39563065A/G
6-4: 39570752C/A
6-4: 39589148A/C
6-4: 39683333G/A
6-4: 39827630C/T
6-4: 39830520C/T
6-4: 39863459A/G
6-4: 39925778T/A
6-4: 40035267G/T
6-4: 40075881A/G
6-4: 40135435A/T
6-4: 40136992G/C
6-4: 40137079C/T
6-4: 40282051C/T
6-4: 40314534G/T
6-4: 40396765T/C
6-4: 40435472C/T
6-4: 40570338G/A
6-4: 40641238T/C
6-4: 40723725G/A
6-4: 40776304G/A
6-4: 40969322T/G
6-4: 40977711A/C
6-4: 41038323G/T
6-4: 41109251A/G
6-4: 41109398C/A
6-4: 41110956A/G
6-4: 41123432C/T
6-4: 41148526T/G
6-4: 41158615G/A
6-4: 41269196A/G
6-4: 41331475C/A
6-4: 41363074A/C
6-4: 41513935T/A
6-4: 41548797G/A
6-4: 41577533A/T
6-4: 41607957T/C
6-4: 41645761A/G
6-4: 41646133C/A
6-4: 41646244G/T
6-4: 41868876C/T
6-4: 41959242T/A
6-4: 42014039G/A
6-4: 42140747C/T
6-4: 42185361C/T
6-4: 42188668G/C
6-4: 42227027T/C
6-4: 42230646T/C
6-4: 42230813G/A
6-4: 42230929T/A
6-4: 42255047G/C
6-4: 42255771T/C
6-4: 42258438C/G
6-4: 42307373C/T
6-4: 42320643C/A
6-4: 42392087C/T
6-4: 42406666C/T
6-4: 42455483C/A
6-4: 42496887C/T
6-4: 42546911A/G
6-4: 42580118T/A
6-4: 42630437G/A
6-4: 42651110T/A
6-4: 42670482A/G
6-4: 42761575G/A
6-4: 42788900C/G
6-4: 42788993A/T
6-4: 42789291T/A
6-4: 42790851C/A
6-4: 42790857T/C
6-4: 42794475T/C
6-4: 42794581G/A
6-4: 42801461T/C
6-4: 42802530C/T
6-4: 42856011G/A
6-4: 42870866T/G
6-4: 42877626T/A
6-4: 42880473C/T
6-4: 42880544A/C
6-4: 42880568A/T
6-4: 42883587A/G
6-4: 42884001C/T
6-4: 42884089G/A
6-4: 42884131G/A
6-4: 42884160C/T
6-4: 42886069A/G
6-4: 42905992T/G
6-4: 42917518A/G
6-4: 42922726G/T
6-4: 42948660T/G
6-4: 42950584G/C
6-4: 42950703T/G
6-4: 42957304C/T
6-4: 42957734G/T
6-4: 42957761G/T
6-4: 42963775G/T
6-4: 42967022A/T
6-4: 42967036G/A
6-4: 42967192C/T
6-4: 42977625A/T
6-4: 42986521A/T
6-4: 43017019G/T
6-4: 43066436G/A
6-4: 43127866C/A
6-4: 43162133A/G
6-4: 43182930T/G
6-4: 43196622C/T
6-4: 43235291T/C
6-4: 43296412C/T
6-4: 43353335T/C
6-4: 43411074A/G
6-4: 43450969A/G
6-4: 43466645T/A
6-4: 43530498C/T
6-4: 43569384A/G
6-4: 43631881A/G
6-4: 43644444T/C
6-4: 43694016T/C
6-4: 43700226T/A
6-4: 43744671C/T
6-4: 43796885A/G
6-4: 43809297G/A
6-4: 43826770G/T
6-4: 43846819C/G
6-4: 43902409G/A
6-4: 43930809C/T
6-4: 43988827T/A
6-4: 44017122T/A
6-4: 44137964T/C
6-4: 44185751C/T
6-4: 44205361C/A
6-4: 44335550T/G
6-4: 44336890G/A
6-4: 44348192C/G
6-4: 44364379G/A
6-4: 44389673C/T
6-4: 44435250C/T
6-4: 44458133T/A
6-4: 44546649G/A
6-4: 44994164C/T
6-4: 45163434G/T
6-4: 45188314A/G
6-4: 45466952A/G
6-4: 45472487T/A
6-4: 45505019G/T
6-4: 45569916T/G
6-4: 45580251C/A
6-4: 45590715A/G
6-4: 45858564G/A
6-4: 45859763C/T
6-4: 45887987G/T
6-4: 45898896C/T
6-4: 45899091A/T
6-4: 45982308G/T
6-4: 46015964C/T
6-4: 46067863C/T
6-4: 46108600G/C
6-4: 46161866G/A
6-4: 46302632A/G
6-4: 46334561G/A
6-4: 46396395C/T
6-4: 46432095C/A
6-4: 46521180T/G
6-4: 46717538A/G
6-4: 46730730A/T
6-4: 46755583T/A
6-4: 46816911C/G
6-4: 46858169C/A
6-4: 46904355C/T
6-4: 46979583C/T
6-4: 46990242C/T
6-4: 47093170G/A
6-4: 47143814A/G
6-4: 47175834A/G
6-4: 47262908T/G
6-4: 47288692G/A
6-4: 47359459T/A
6-4: 47391485G/T
6-4: 47408099C/T
6-4: 47445027C/A
6-4: 47560207A/T
6-4: 47615325G/A
6-4: 47695615C/T
6-4: 47706711T/C
6-4: 47754578T/A
6-4: 47786352T/C
6-4: 47825051T/C
6-4: 47835494C/T
6-4: 47911932C/T
6-4: 47986622T/A
6-4: 48083208G/A
6-4: 48113441C/T
6-4: 48282761G/T
6-4: 48390767T/A
6-4: 48525647A/T
6-4: 48595391T/A
6-4: 48625337T/C
6-4: 48690505G/A
6-4: 48711019A/T
6-4: 48726444A/G
6-4: 48746723T/C
6-4: 48800037T/G
6-4: 48988396T/C
6-4: 49043174A/G
6-4: 49131522C/T
6-4: 49259041A/G
6-4: 49352713T/C
6-4: 49422223A/G
6-4: 49688284A/G
6-4: 49746497C/G
6-4: 49778135C/T
6-4: 49859911G/A
6-4: 49905001A/C
6-4: 50093559A/G
6-4: 50322378G/A
6-4: 50399061T/C
6-4: 50403015T/G
6-4: 50442386T/C
6-4: 50459729T/C
6-4: 50543905G/T
6-4: 50594123T/G
6-4: 50696709A/C
6-4: 50713499A/G
6-4: 50724759A/G
6-4: 50960089A/G
6-4: 51054335G/A
6-4: 51138555T/C
6-4: 51179680C/T
6-4: 51288801G/A
6-4: 51468993C/A
6-4: 51480494T/G
6-4: 51491889A/T
6-4: 51502168G/A
6-4: 51505596T/A
6-4: 51529024T/A
6-4: 51540312G/A
6-4: 51646846C/T
6-4: 51694072C/T
6-4: 51732557A/G
6-4: 51894257A/T
6-4: 51913657G/A
6-4: 51952934C/T
6-4: 52087202G/T
6-4: 52088725G/A
6-4: 52147345G/A
6-4: 52257923T/A
6-4: 52288465T/C
6-4: 52296087T/C
6-4: 52300951A/G
6-4: 52312767A/T
6-4: 52324815T/C
6-4: 52327607A/G
6-4: 52430921G/A
6-4: 52458477T/C
6-4: 52474690C/T
6-4: 52531766G/A
6-4: 52549716G/T
6-4: 52566565T/C
6-4: 52566734C/T
6-4: 52567542G/A
6-4: 52616783C/T
6-4: 52651492T/C
6-4: 52749881C/A
6-4: 52761967T/G
6-4: 52791097G/C
6-4: 52915023T/G
6-4: 53003781T/C
6-4: 53013893A/G
6-4: 53014018T/G
6-4: 53015773C/T
6-4: 53016418A/G
6-4: 53016845C/T
6-4: 53058252T/C
6-4: 53188164A/G
6-4: 53298169A/G
6-4: 53476063C/G
6-4: 53641574T/A
6-4: 53836881A/C
6-4: 53882791A/G
6-4: 53986401T/A
6-4: 54004409T/G
6-4: 54056289A/G
6-4: 54066626C/T
6-4: 54076832G/T
6-4: 54087281C/T
6-4: 54108532T/A
6-4: 54161350T/G
6-4: 54197672G/T
6-4: 54220823C/T
6-4: 54237936A/T
6-4: 54274902C/T
6-4: 54281317G/A
6-4: 54281327C/T
6-4: 54281562C/A
6-4: 54281622G/T
6-4: 54281878T/C
6-4: 54282028C/T
6-4: 54282125G/A
6-4: 54282132C/T
6-4: 54282340T/A
6-4: 54282482G/A
6-4: 54282754G/T
6-4: 54283852G/A
6-4: 54285380A/C
6-4: 54302534T/A
6-4: 54376843C/T
6-4: 54557277A/G
6-4: 54629923A/G
6-4: 54725770C/G
6-4: 54832032A/G
6-4: 54833331C/G
6-4: 54834097G/T
6-4: 54894000A/T
6-4: 54941918A/T
6-4: 54944034C/T
6-4: 54993756T/C
6-4: 55048260G/A
6-4: 55080890G/A
6-4: 55100139G/C
6-4: 55100710G/A
6-4: 55101678C/A
6-4: 55102056A/T
6-4: 55102210C/T
6-4: 55102376C/A
6-4: 55102517A/G
6-4: 55118959C/T
6-4: 55166843C/T
6-4: 55209589A/G
6-4: 55237655G/C
6-4: 55296505G/A
6-4: 55404183T/A
6-4: 55506153T/C
6-4: 55565040A/C
6-4: 55578685A/G
6-4: 55592659C/A
6-4: 55617344G/A
6-4: 55681611A/G
6-4: 55735367G/T
6-4: 55772873C/A
6-4: 55784169A/T
6-4: 55804133T/A
6-4: 55954577T/C
6-4: 56031899C/A
6-4: 56034876A/T
6-4: 56071269T/A
6-4: 56081759A/G
6-4: 56093216A/G
6-4: 56127794A/G
6-4: 56138829A/G
6-4: 56146870A/C
6-4: 56158843T/A
6-4: 56166890A/G
6-4: 56170391C/T
6-4: 56181640C/T
6-4: 56192370A/G
6-4: 56212227A/T
6-4: 56222491T/C
6-4: 56235144C/T
6-4: 56263860A/G
6-4: 56275785G/T
6-4: 56306325G/C
6-4: 56381690C/A
6-4: 56412331G/A
6-4: 56442710C/T
6-4: 56721891A/G
6-4: 56738952G/T
6-4: 56775187G/A
6-4: 56801929T/C
6-4: 57036589C/T
6-4: 57077564C/T
6-4: 57092387A/G
6-4: 57130250A/G
6-4: 57198131A/G
6-4: 57238942G/A
6-4: 57272967C/A
6-4: 57305812T/C
6-4: 57321439C/T
6-4: 57342685C/A
6-4: 57359708G/T
6-4: 57368272G/A
6-4: 57368279T/A
6-4: 57388925G/A
6-4: 57400523C/A
6-4: 57415556C/T
6-4: 57415621C/T
6-4: 57415652A/G
6-4: 57644204G/A
6-4: 57676359G/T
6-4: 57703597A/C
6-4: 57784882A/T
6-4: 57840199T/C
6-4: 57958841T/A
6-4: 58064281A/G
6-4: 58189758G/A
6-4: 58191142G/A
6-4: 58193081C/T
6-4: 58295894A/C
6-4: 58332684T/C
6-4: 58347759T/C
6-4: 58473103G/A
6-4: 58595282G/A
6-4: 58652379G/A
6-4: 58918279G/A
6-4: 58987318C/T
6-4: 59050539A/G
6-4: 59119042C/T
6-4: 59169778T/A
6-4: 59185216T/C
6-4: 59272600G/A
6-4: 59308465G/A
6-4: 59355031A/T
6-4: 59516937A/T
6-4: 59589264T/C
6-4: 59663330A/T
6-4: 59709738C/A
6-4: 59769625G/A
6-4: 59789247C/T
6-4: 59802106G/A
6-4: 59871307G/A
6-4: 59941621G/A
6-4: 60051102G/C
6-4: 60063436A/G
6-4: 60141963T/A
6-4: 60175574T/C
6-4: 60190823G/A
6-4: 60283519A/T
6-4: 60342608G/T
6-4: 60456491T/C
6-4: 60457657G/A
6-4: 60459285G/A
6-4: 60466708T/C
6-4: 60485116C/G
6-4: 60534798A/T
6-4: 60546952C/A
6-4: 60560562C/T
6-4: 60624128T/A
6-4: 60804136T/C
6-4: 60886110T/C
6-4: 60896664C/T
6-4: 60979790G/A
6-4: 61016528A/T
6-4: 61039852C/T
6-4: 61175992T/A
6-4: 61191807G/T
6-4: 61327791T/A
6-4: 61537973C/T
6-4: 61594904C/T
6-4: 61606636T/C
6-4: 61622358A/C
6-4: 61663085A/C
6-4: 61669745G/T
6-4: 61669764T/A
6-4: 61886847C/T
6-4: 61897492T/C
6-4: 61959050G/T
6-4: 61999769A/G
6-4: 62092855C/T
6-4: 62108789G/A
6-4: 62195998T/C
6-4: 62216870T/C
6-4: 62277985T/G
6-4: 62514120G/A
6-4: 62545716A/G
6-4: 62560260A/C
6-4: 62653487A/T
6-4: 62711818A/C
6-4: 62773086G/A
6-4: 62788782C/T
6-4: 62831113C/T
6-4: 62846507A/C
6-4: 63017053C/T
6-4: 63039082C/T
6-4: 63148394C/G
6-4: 63159325C/T
6-4: 63185291T/C
6-4: 63260768C/T
6-4: 63316288T/C
6-4: 63358051T/G
6-4: 63387942A/C
6-4: 63463272G/A
6-4: 63510830C/T
6-4: 63561204C/T
6-4: 63582385C/T
6-4: 63672746G/A
6-4: 63674495T/G
6-4: 63686325A/G
6-4: 63726575A/G
6-4: 63739507C/T
6-4: 63841639C/T
6-4: 63884359C/T
6-4: 63925799A/C
6-4: 63978117C/T
6-4: 64095218T/G
6-4: 64169125A/C
6-4: 64238235A/T
6-4: 64282444A/G
6-4: 64366554T/C
6-4: 64445109C/T
6-4: 64458613C/A
6-4: 64641560A/T
6-4: 64674142T/C
6-4: 64689840T/G
6-4: 64709440A/G
6-4: 64742404C/T
6-4: 64775359C/G
6-4: 64856895G/T
6-4: 64947487G/T
6-4: 65047391A/G
6-4: 65067283A/G
6-4: 65100361A/G
6-4: 65135055A/T
6-4: 65205512T/G
6-4: 65274844G/A
6-4: 65337706A/G
6-4: 65354663C/T
6-4: 65364713C/A
6-4: 65379382A/G
6-4: 65393044T/C
6-4: 65414767A/C
6-4: 65442422C/A
6-4: 65479510T/C
6-4: 65494855T/G
6-4: 65505256A/G
6-4: 65515273T/G
6-4: 65544142C/G
6-4: 65544244G/T
6-4: 65544424T/A
6-4: 65544564C/T
6-4: 65547850A/C
6-4: 65572585A/T
6-4: 65611822C/T
6-4: 65672686G/A
6-4: 65709341C/T
6-4: 65781736T/G
6-4: 65799289T/C
6-4: 65799441A/T
6-4: 65799547G/T
6-4: 65821468C/A
6-4: 65867669G/T
6-4: 65868604G/A
6-4: 65883939A/G
6-4: 65904854A/T
6-4: 65905826C/A
6-4: 65919089A/G
6-4: 65954547C/A
6-4: 65959089G/A
6-4: 66003627C/G
6-4: 66032379G/A
6-4: 66104496C/T
6-4: 66146891T/C
6-4: 66195871T/G
6-4: 66235965G/A
6-4: 66361131T/A
6-4: 66365236A/C
6-4: 66383759T/C
6-4: 66410355A/G
6-4: 66548137T/C
6-4: 66556510C/T
6-4: 66583093A/T
6-4: 66584510C/A
6-4: 66627893G/A
6-4: 66682379A/G
6-4: 66746811C/A
6-4: 66942496C/T
6-4: 66979689T/G
6-4: 67041177A/T
6-4: 67057514A/G
6-4: 67246982T/C
6-4: 67372849G/A
6-4: 67428576A/G
6-4: 67456159A/C
6-4: 67466917G/A
6-4: 67477775A/G
6-4: 67495909C/G
6-4: 67515635G/A
6-4: 67562409G/T
6-4: 67666071C/T
6-4: 67680958A/G
6-4: 67717562G/T
6-4: 67746809G/A
6-4: 67772740T/A
6-4: 67784158G/T
6-4: 67802145T/C
6-4: 67819421A/T
6-4: 67923177T/C
6-4: 67924262G/T
6-4: 67926273T/C
6-4: 67926296A/T
6-4: 67926527C/T
6-4: 67926553T/C
6-4: 67926711G/A
6-4: 67926737C/T
6-4: 67927044G/A
6-4: 67927271C/T
6-4: 67927342G/A
6-4: 67927544G/A
6-4: 67927594G/A
6-4: 67927722G/C
6-4: 67927829G/A
6-4: 67927952C/T
6-4: 67927997C/T
6-4: 67928335C/T
6-4: 67963617C/A
6-4: 67965056C/A
6-4: 68031147G/A
6-4: 68072452T/C
6-4: 68089098G/A
6-4: 68100226T/C
6-4: 68113211A/G
6-4: 68309504A/G
6-4: 68384614G/A
6-4: 68419660G/T
6-4: 68514395A/C
6-4: 68542857T/C
6-4: 68544806A/T
6-4: 68547423C/T
6-4: 68683383T/C
6-4: 68695427T/C
6-4: 68714421C/A
6-4: 68726631C/T
6-4: 68754192G/A
6-4: 68755152T/C
6-4: 68787980A/G
6-4: 68815076A/G
6-4: 68859374A/G
6-4: 68887000T/C
6-4: 68916797C/T
6-4: 68927341C/G
6-4: 68963145A/G
6-4: 69018386C/T
6-4: 69072356T/A
6-4: 69084764C/T
6-4: 69116441A/G
6-4: 69246726T/C
6-4: 69279231A/G
6-4: 69297203T/G
6-4: 69307649C/A
6-4: 69441751C/T
6-4: 69442952G/A
6-4: 69454171T/G
6-4: 69465632C/G
6-4: 69541615A/G
6-4: 69570246C/T
6-4: 69685464T/A
6-4: 69695957T/C
6-4: 69716522G/T
6-4: 69728410A/T
6-4: 69743095T/A
6-4: 69813851T/C
6-4: 69885439T/C
6-4: 69909615T/C
6-4: 69940092T/A
6-4: 69952624T/A
6-4: 69973122C/T
6-4: 70005638A/T
6-4: 70081136C/T
6-4: 70126546G/C
6-4: 70137574G/A
6-4: 70162382C/T
6-4: 70193502T/A
6-4: 70196855A/G
6-4: 70196919C/T
6-4: 70196947C/G
6-4: 70197379T/A
6-4: 70197483T/C
6-4: 70198488T/C
6-4: 70206879C/T
6-4: 70221894C/T
6-4: 70222986G/A
6-4: 70222987A/T
6-4: 70223452A/G
6-4: 70249013C/T
6-4: 70322709A/G
6-4: 70354749C/T
6-4: 70381887A/G
6-4: 70505762G/A
6-4: 70516634A/G
6-4: 70553732G/T
6-4: 70590307A/T
6-4: 70643720C/T
6-4: 70660108C/T
6-4: 70687715A/T
6-4: 70776401A/G
6-4: 70888642C/T
6-4: 70894993C/T
6-4: 70910280A/G
6-4: 70954754A/G
6-4: 71003898G/A
6-4: 71047276G/A
6-4: 71117244A/G
6-4: 71210952T/C
6-4: 71213025T/C
6-4: 71233087T/C
6-4: 71275442C/T
6-4: 71308660T/C
6-4: 71395043G/C
6-4: 71535875G/C
6-4: 71564248A/G
6-4: 71575182A/C
6-4: 71586117A/T
6-4: 71599240T/C
6-4: 71777121A/T
6-4: 71876791T/A
6-4: 71876937G/C
6-4: 71876968C/T
6-4: 71876983T/A
6-4: 71877029A/G
6-4: 72034206T/A
6-4: 72171862A/C
6-4: 72328828G/A
6-4: 72392343G/A
6-4: 72392497G/T
6-4: 72403820C/A
6-4: 72450640T/C
6-4: 72480577G/A
6-4: 72513906G/A
6-4: 72547708C/T
6-4: 72591025A/T
6-4: 72606914T/G
6-4: 72618011T/A
6-4: 72731863G/A
6-4: 72763349C/T
6-4: 72777626T/G
6-4: 72842906C/T
6-4: 72844430G/C
6-4: 72845704C/T
6-4: 72930478T/G
6-4: 72940598A/G
6-4: 72953719C/T
6-4: 73078956C/A
6-4: 73089782C/T
6-4: 73102422A/T
6-4: 73135472C/T
6-4: 73201518T/G
64: 73228181G/C
6-4: 73280942T/C
6-4: 73386188C/G
6-4: 73414748T/C
6-4: 73431732A/T
6-4: 73451606G/A
6-4: 73505233C/A
6-4: 73517273T/C
6-4: 73563296G/A
6-4: 73586845T/A
6-4: 73611239T/C
6-4: 73675548G/A
6-4: 73703072G/A
6-4: 73722496C/T
6-4: 73750204A/T
6-4: 73757607G/T
6-4: 73759354G/A
6-4: 73759375G/A
6-4: 73759466G/A
6-4: 73760727G/T
6-4: 73760903G/A
6-4: 73761228T/A
6-4: 73762855G/A
6-4: 73773930C/T
6-4: 73774950G/A
6-4: 73779720G/T
6-4: 73779838C/T
6-4: 73780829G/A
6-4: 73813266G/A
6-4: 73825215A/T
6-4: 73852992C/T
6-4: 73892147G/T
6-4: 74023293C/T
6-4: 74023997G/A
6-4: 74024123G/T
6-4: 74028253G/A
6-4: 74029021C/T
6-4: 74029134G/A
6-4: 74051713A/T
6-4: 74060779T/C
6-4: 74112057T/C
6-4: 74164018C/A
6-4: 74212907C/A
6-4: 74263598A/G
6-4: 74282179G/A
6-4: 74299728C/T
6-4: 74312733A/T
6-4: 74339952A/G
6-4: 74379990A/G
6-4: 74423463T/G
6-4: 74439596G/A
6-4: 74506127A/G
6-4: 74519341A/T
6-4: 74529944C/T
6-4: 74558386A/G
6-4: 74574205G/A
6-4: 74605947T/G
6-4: 74705204T/C
6-4: 74831884C/T
6-4: 74839689A/T
6-4: 74871214T/C
6-4: 74895767T/A
6-4: 74944237C/G
6-4: 74976186T/C
6-4: 75001738C/T
6-4: 75013770G/A
6-4: 75026006G/A
6-4: 75069577T/G
6-4: 75111821A/T
6-4: 75132008C/.
6-4: 75149180T/G
6-4: 75200098C/G
6-4: 75221146G/A
6-4: 75253503C/A
6-4: 75253734T/C
6-4: 75274841T/C
6-4: 75289117G/A
6-4: 75308938A/G
6-4: 75338727A/T
6-4: 75397908G/A
6-4: 75435705T/C
6-4: 75454447A/C
6-4: 75491534T/C
6-4: 75545460T/C
6-4: 75561955A/T
6-4: 75590284A/C
6-4: 75621310T/A
6-4: 75660499G/A
6-4: 75744052A/C
6-4: 75757679C/T
6-4: 75810630G/T
6-4: 75880838C/T
6-4: 75886357T/C
6-4: 75889381A/T
6-4: 75889635G/T
6-4: 75913066C/A
6-4: 75916993C/A
6-4: 75932956T/A
6-4: 75943099A/T
6-4: 75964588A/T
6-4: 75972156A/G
6-4: 75976349C/T
6-4: 75988645G/T
6-4: 75989741G/A
6-4: 76006927G/A
6-4: 76017258A/T
6-4: 76017359T/C
6-4: 76017950G/T
6-4: 76018883C/A
6-4: 76019176G/A
6-4: 76019224G/T
6-4: 76019344A/T
6-4: 76019430G/A
6-4: 76039636T/C
6-4: 76045398A/T
6-4: 76049977A/G
6-4: 76064630T/C
6-4: 76074911A/T
6-4: 76080805G/T
6-4: 76080949A/G
6-4: 76081049T/C
6-4: 76086185C/T
6-4: 76086838A/G
6-4: 76098567G/C
6-4: 76111835A/G
6-4: 76112935T/A
6-4: 76113242T/A
6-4: 76113312T/A
6-4: 76113563A/G
6-4: 76120903G/T
6-4: 76122272A/G
6-4: 76163835A/G
6-4: 76174503C/T
6-4: 76188584A/C
6-4: 76210613G/C
6-4: 76216788G/T
6-4: 76218885A/G
6-4: 76219275G/A
6-4: 76221019A/T
6-4: 76221354A/G
6-4: 76221420G/A
6-4: 76221424G/A
6-4: 76221521A/C
6-4: 76221731C/T
6-4: 76221965T/A
6-4: 76223327C/T
6-4: 76223866T/G
6-4: 76223980A/T
6-4: 76224759T/A
6-4: 76226519C/T
6-4: 76226569C/T
6-4: 76228075C/T
6-4: 76228223G/A
6-4: 76228351C/T
6-4: 76229749A/T
6-4: 76230403A/G
6-4: 76230405T/C
6-4: 76230514G/A
6-4: 76230608C/T
6-4: 76230621T/A
6-4: 76231531G/A
6-4: 76231861T/C
6-4: 76231969A/C
6-4: 76232041A/T
6-4: 76232073G/A
6-4: 76232246T/C
6-4: 76232474T/C
6-4: 76233023C/G
6-4: 76233068C/T
6-4: 76233131T/C
6-4: 76233256C/T
6-4: 76233380A/T
6-4: 76233559C/T
6-4: 76235026T/G
6-4: 76235040T/C
6-4: 76235452A/G
6-4: 76241416G/A
6-4: 76338774C/T
6-4: 76354338A/G
6-4: 76396008C/T
6-4: 76427039T/C
6-4: 76442251T/C
6-4: 76489292G/A
6-4: 76508517G/T
6-4: 76516419G/A
6-4: 76516497G/A
6-4: 76517084C/T
6-4: 76520903C/T
6-4: 76525908C/T
6-4: 76531077C/G
6-4: 76531660A/G
6-4: 76561534G/A
6-4: 76572625A/G
6-4: 76592874G/A
6-4: 76615816T/C
6-4: 76626419T/C
6-4: 76730306T/A
6-4: 76785444C/T
6-4: 76969354T/G
6-4: 76992260C/T
6-4: 76993589C/T
6-4: 77012588T/C
6-4: 77029645A/G
6-4: 77138331G/A
6-4: 77191872C/T
6-4: 77204398G/T
6-4: 77231158C/A
6-4: 77250834T/A
6-4: 77315366T/C
6-4: 77325639G/T
6-4: 77375859G/C
6-4: 77477912A/T
6-4: 77486553A/G
6-4: 77507088A/G
6-4: 77532160G/C
6-4: 77593189C/T
6-4: 77636819A/C
6-4: 77662003C/T
6-4: 77714130C/G
6-4: 77803171A/C
6-4: 77845600G/T
6-4: 77848080T/C
6-4: 77848380C/T
6-4: 77868999T/A
6-4: 77911230A/T
6-4: 77944111A/G
6-4: 77944586G/T
6-4: 78049763T/C
6-4: 78049876T/C
6-4: 78062429A/G
6-4: 78062442A/T
6-4: 78092782G/A
6-4: 78099174A/G
6-4: 78115637T/C
6-4: 78148138T/A
6-4: 78192605G/A
6-4: 78249173A/T
6-4: 78321429A/T
6-4: 78370707T/A
6-4: 78384370A/G
6-4: 78429279C/T
6-4: 78439448A/G
6-4: 78468302C/T
6-4: 78510135G/C
6-4: 78567218C/G
6-4: 78663121A/T
6-4: 78672187C/T
6-4: 78683242C/T
6-4: 78696369C/T
6-4: 78708970C/G
6-4: 78750335G/A
6-4: 78828678G/T
6-4: 78840679T/C
6-4: 78845943T/C
6-4: 78866172G/A
6-4: 79036272A/G
6-4: 79051579A/T
6-4: 79055421A/T
6-4: 79058841A/G
6-4: 79058843A/G
6-4: 79133046C/T
6-4: 79209788T/G
6-4: 79312482C/T
6-4: 79347829T/C
6-4: 79372617A/G
6-4: 79389834T/G
6-4: 79409060G/A
6-4: 79422565A/G
6-4: 79424171G/C
6-4: 79443460T/C
6-4: 79476559T/A
6-4: 79481011T/A
6-4: 79521368G/A
6-4: 79522386C/T
6-4: 79536541G/A
6-4: 79562265T/A
6-4: 79570147G/T
6-4: 79573174T/G
6-4: 79588618A/G
6-4: 79599637A/T
6-4: 79612630C/T
6-4: 79634084T/C
6-4: 79643121A/T
6-4: 79644597T/C
6-4: 79644598T/G
6-4: 79645374G/A
6-4: 79654403C/T
6-4: 79663555G/A
6-4: 79674475T/C
6-4: 79769770C/T
6-4: 79785769T/A
6-4: 79831818G/A
6-4: 79927753T/C
6-4: 79976860G/A
6-4: 80006114G/C
6-4: 80066612A/G
6-4: 80104208C/T
6-4: 80143716A/G
6-4: 80148444T/C
6-4: 80155868G/A
6-4: 80178382A/G
6-4: 80189393C/T
6-4: 80201837A/G
6-4: 80220669T/C
6-4: 80238057A/G
6-4: 80258647T/C
6-4: 80296932T/A
6-4: 80307504C/T
6-4: 80354803A/T
6-4: 80366090A/T
6-4: 80381322G/T
6-4: 80419883A/T
6-4: 80445121T/C
6-4: 80541817G/C
6-4: 80546580T/C
6-4: 80577526A/G
6-4: 80579905C/T
6-4: 80674521C/G
6-4: 80761070A/C
6-4: 80764937G/A
6-4: 80772635G/T
6-4: 80772640C/A
6-4: 80799277A/G
6-4: 80860816T/C
6-4: 80893784G/A
6-4: 80921395A/G
6-4: 80931912C/G
6-4: 80957654C/G
6-4: 81001033A/G
6-4: 81060551T/C
6-4: 81061692G/C
6-4: 81076891G/C
6-4: 81088903T/C
6-4: 81107648C/T
6-4: 81119400G/C
6-4: 81270645A/G
6-4: 81410624T/C
6-4: 81412196T/A
6-4: 81426699G/A
6-4: 81487536C/T
6-4: 81499574G/A
6-4: 81522129A/C
6-4: 81523250G/A
6-4: 81523649T/G
6-4: 81534510A/G
6-4: 81599606C/T
6-4: 81724833G/A
6-4: 81765715G/T
6-4: 81787259T/G
6-4: 81801669G/A
6-4: 81803544G/A
6-4: 81803673T/C
6-4: 81804404G/A
6-4: 81804499C/A
6-4: 81805793T/A
6-4: 81806032C/A
6-4: 81831842G/A
6-4: 81833088G/A
6-4: 81844899C/A
6-4: 81891898C/T
6-4: 81903246T/A
6-4: 82085713T/C
6-4: 82154689C/T
6-4: 82283293T/C
6-4: 82386250A/G
6-4: 82396290T/A
6-4: 82408596C/T
6-4: 82457932A/T
6-4: 82459553G/A
6-4: 82486750T/A
6-4: 82501685G/T
6-4: 82536071G/A
6-4: 82554198G/A
6-4: 82554741G/T
6-4: 82584385T/C
6-4: 82600008C/A
6-4: 82643128C/T
6-4: 82705553C/T
6-4: 82722775A/G
6-4: 82779021A/G
6-4: 82848079T/C
6-4: 82908381A/T
6-4: 82969305T/C
6-4: 83100098G/A
6-4: 83123883G/C
6-4: 83178666T/A
6-4: 83185235T/C
6-4: 83191589G/T
6-4: 83236259G/A
6-4: 83335847T/G
6-4: 83394505A/.
6-4: 83394600T/A
6-4: 83426103G/A
6-4: 83463974G/A
6-4: 83506869T/C
6-4: 83534630C/A
6-4: 83583365T/C
6-4: 83625288C/T
6-4: 83714841A/T
6-4: 83778041G/A
6-4: 83819872G/A
6-4: 83914214G/T
6-4: 83926779A/T
6-4: 83987388T/C
6-4: 83989715G/A
6-4: 84031840C/A
6-4: 84074919T/A
6-4: 84118432C/A
6-4: 84200876G/T
6-4: 84233722C/T
6-4: 84256237C/A
6-4: 84319232G/A
6-4: 84359080A/G
6-4: 84424577T/C
6-4: 84514008C/A
6-4: 84579155A/C
6-4: 84614262C/T
6-4: 84722587T/C
6-4: 84751477C/T
6-4: 84771574T/C
6-4: 84817048C/T
6-4: 84853696C/A
6-4: 84907379C/G
6-4: 84944997G/T
6-4: 84945434C/T
6-4: 84970422T/C
6-4: 85053155C/T
6-4: 85154090A/G
6-4: 85165322C/T
6-4: 85170104C/T
6-4: 85242167G/A
6-4: 85253765C/A
6-4: 85278181G/A
6-4: 85310519T/C
6-4: 85397713A/G
6-4: 85420284G/A
6-4: 85619376T/C
6-4: 85719740A/C
6-4: 85943182T/G
6-4: 85954652G/T
6-4: 86133601G/T
6-4: 86197241G/A
6-4: 86478436G/A
6-4: 86552121T/C
6-4: 86792768C/A
6-4: 86796546C/T
6-4: 86955733A/G
6-4: 86960867C/T
6-4: 87307804A/T
6-4: 87344467G/A
6-4: 87405090G/A
6-4: 87566051C/T
6-4: 87965009T/A
6-4: 87966050T/A
6-4: 88000841C/G
6-4: 88072439G/A
6-4: 88090168A/G
6-4: 88184985A/G
6-4: 88250851A/G
6-4: 88256781C/G
6-4: 88283626T/C
6-4: 88299286T/A
6-4: 88946198T/A
6-4: 88966155T/G
6-4: 88976181G/A
6-4: 89040625T/C
6-4: 89069838C/G
6-4: 89314941C/T
6-4: 89347354A/G
6-4: 89363467C/G
6-4: 89373814C/G
6-4: 89687292A/T
7-1: 246669A/C
7-1: 278262T/A
7-1: 289337G/A
7-1: 414992C/T
7-1: 511306G/A
7-1: 628304C/A
7-1: 806535A/G
7-1: 928059T/C
7-1: 942854C/T
7-1: 1015663C/A
7-1: 1030193C/T
7-1: 1065836A/T
7-1: 1105631T/A
7-1: 1128498T/A
7-1: 1147251C/T
7-1: 1209393C/T
7-1: 1302041G/A
7-1: 1383906C/T
7-1: 1433624A/G
7-1: 1517730T/G
7-1: 1647973G/A
7-1: 1730104T/A
7-1: 1833296T/G
7-1: 1833435A/T
7-1: 1860113G/A
7-1: 1889893C/A
7-1: 1936844C/T
7-1: 1954152A/G
7-1: 1960614A/G
7-1: 2043776C/T
7-1: 2080780G/A
7-1: 2111882C/T
7-1: 2292865T/G
7-1: 2352290T/A
7-1: 2397916A/G
7-1: 2588110G/A
7-1: 2608275A/T
7-1: 2611147G/A
7-1: 2635064G/C
7-1: 2653510A/G
7-1: 2757837A/G
7-1: 2797941C/T
7-1: 2881972A/T
7-1: 2929569C/A
7-1: 2930311G/C
7-1: 2930719T/C
7-1: 2931373A/G
7-1: 2953350C/T
7-1: 2960366T/A
7-1: 2964706T/A
7-1: 2976845A/C
7-1: 3003679T/C
7-1: 3051049G/A
7-1: 3052080T/A
7-1: 3052403T/A
7-1: 3075053G/A
7-1: 3091008G/C
7-1: 3093340G/T
7-1: 3094284C/T
7-1: 3096233T/A
7-1: 3104238C/T
7-1: 3106178C/A
7-1: 3132190C/T
7-1: 3147887A/G
7-1: 3148025A/T
7-1: 3148059T/G
7-1: 3172629T/C
7-1: 3204250A/G
7-1: 3287498A/T
7-1: 3371812C/A
7-1: 3418413C/G
7-1: 3441465A/T
7-1: 3653904G/C
7-1: 3689367T/A
7-1: 3766447A/T
7-1: 3767533A/G
7-1: 3802957A/C
7-1: 3828114T/C
7-1: 3860303A/T
7-1: 3913989G/A
7-1: 3914259A/C
7-1: 3915802A/G
7-1: 3996811C/T
7-1: 4003631C/T
7-1: 4030257C/G
7-1: 4083038T/A
7-1: 4136495C/T
7-1: 4292959T/C
7-1: 4311573T/C
7-1: 4472381A/T
7-1: 4528243T/A
7-1: 4556260C/G
7-1: 4583542C/A
7-1: 4616241A/T
7-1: 4644571A/G
7-1: 4724285C/T
7-1: 4737459C/T
7-1: 4790808T/A
7-1: 4847071G/A
7-1: 4854566C/T
7-1: 4858347C/T
7-1: 4901890A/G
7-1: 4985733C/G
7-1: 4986134A/G
7-1: 5005967A/C
7-1: 5291174G/A
7-1: 5346716A/T
7-1: 5399505A/T
7-1: 5470650T/C
7-1: 5497824T/C
7-1: 5498426G/T
7-1: 5512151C/T
7-1: 5524202T/C
7-1: 5546047C/T
7-1: 5578168T/C
7-1: 5618751A/C
7-1: 5675281A/G
7-1: 5697735T/C
7-1: 5726407A/G
7-1: 5786039C/G
7-1: 5824018G/A
7-1: 5919991T/C
7-1: 5935101G/A
7-1: 5987759G/T
7-1: 6203608A/G
7-1: 6258002A/C
7-1: 6346560T/G
7-1: 6358439C/G
7-1: 6358911A/G
7-1: 6415897G/A
7-1: 6448517A/G
7-1: 6509336G/T
7-1: 6562963A/G
7-1: 6608924T/C
7-1: 6640171T/A
7-1: 6662507T/G
7-1: 6666344G/A
7-1: 6672535A/G
7-1: 6672691G/C
7-1: 6676487T/C
7-1: 6676648G/C
7-1: 6677336C/T
7-1: 6688523A/G
7-1: 6714898T/A
7-1: 6742644C/T
7-1: 6828826T/A
7-1: 6854676T/C
7-1: 6856019C/T
7-1: 6898608A/G
7-1: 6947083G/A
7-1: 6982282T/C
7-1: 7075648G/A
7-1: 7081522G/A
7-1: 7081561G/A
7-1: 7099408G/A
7-1: 7105169C/T
7-1: 7226222G/A
7-1: 7227983C/T
7-1: 7229943A/G
7-1: 7235260A/G
7-1: 7235274C/T
7-1: 7235916T/A
7-1: 7246111G/A
7-1: 7262438T/G
7-1: 7335213G/C
7-1: 7386408G/T
7-1: 7431007G/A
7-1: 7478937C/A
7-1: 7532246G/A
7-1: 7566665C/T
7-1: 7632309G/A
7-1: 7685953T/A
7-1: 7771193A/T
7-1: 7856247A/G
7-1: 7888827G/C
7-1: 7888949A/C
7-1: 7890532G/A
7-1: 7934828G/A
7-1: 7969455A/G
7-1: 8114389A/T
7-1: 8183763G/T
7-1: 8250051A/G
7-1: 8269714T/G
7-1: 8308837G/T
7-1: 8310411G/A
7-1: 8390431T/G
7-1: 8429455C/T
7-1: 8492429T/C
7-1: 8567587G/A
7-1: 8650279A/T
7-1: 8672377G/A
7-1: 8806471T/C
7-1: 8842091A/C
7-1: 8887450G/A
7-1: 8908163G/A
7-1: 8948388T/C
7-1: 9023212C/A
7-1: 9081467T/G
7-1: 9111386C/T
7-1: 9123704A/G
7-1: 9218829G/A
7-1: 9233532T/C
7-1: 9309277T/A
7-1: 9406574G/A
7-1: 9523116G/A
7-1: 9531670C/A
7-1: 9562472T/A
7-1: 9568553T/A
7-1: 9619702C/G
7-1: 9672691G/A
7-1: 9724350T/C
7-1: 9734718T/A
7-1: 9829782A/C
7-1: 9846254T/G
7-1: 9965620T/C
7-1: 10010929A/T
7-1: 10044781C/T
7-1: 10045513T/A
7-1: 10046984G/T
7-1: 10047630T/C
7-1: 10048676A/G
7-1: 10048686G/A
7-1: 10057039T/G
7-1: 10057168C/T
7-1: 10057367C/T
7-1: 10078029A/G
7-1: 10163187T/A
7-1: 10185316C/T
7-1: 10199368C/T
7-1: 10236194G/A
7-1: 10310425C/A
7-1: 10351225C/A
7-1: 10410137C/G
7-1: 10412131A/G
7-1: 10434371G/A
7-1: 10514793C/T
7-1: 10574641T/C
7-1: 10626888C/T
7-1: 10646585C/T
7-1: 10651697C/T
7-1: 10651802T/C
7-1: 10653774T/A
7-1: 10654014C/A
7-1: 10654062T/C
7-1: 10656089G/A
7-1: 10684809C/T
7-1: 10692191G/A
7-1: 10692204C/G
7-1: 10702105C/T
7-1: 10711242G/A
7-1: 10717280C/T
7-1: 10750278A/G
7-1: 10760569T/C
7-1: 10823105T/A
7-1: 10875682G/A
7-1: 10888260A/C
7-1: 10934605T/C
7-1: 10968346G/A
7-1: 11020415A/C
7-1: 11048610T/C
7-1: 11159674C/T
7-1: 11173645A/G
7-1: 11193504C/A
7-1: 11293454A/T
7-1: 11293835A/G
7-1: 11293944C/G
7-1: 11294094A/T
7-1: 11471114A/T
7-1: 11474261T/A
7-1: 11489974A/C
7-1: 11500346C/A
7-1: 11501019C/T
7-1: 11507963G/A
7-1: 11563264C/A
7-1: 11605044G/A
7-1: 11650866G/A
7-1: 11685192C/A
7-1: 11713120T/C
7-1: 11754293A/G
7-1: 11781998G/T
7-1: 11799366T/C
7-1: 11810117A/G
7-1: 11810168G/A
7-1: 11888729G/A
7-1: 11895296C/T
7-1: 11935132G/A
7-1: 12001454C/T
7-1: 12014416A/G
7-1: 12197013G/A
7-1: 12236798T/A
7-1: 12266772G/A
7-1: 12289606G/A
7-1: 12317976C/T
7-1: 12346925G/A
7-1: 12367781A/C
7-1: 12379448G/C
7-1: 12408591A/T
7-1: 12453333T/G
7-1: 12486563C/T
7-1: 12502379T/A
7-1: 12527845G/A
7-1: 12569159G/A
7-1: 12604321G/A
7-1: 12632436C/T
7-1: 12755315T/A
7-1: 12768166G/T
7-1: 12768966C/G
7-1: 12790757T/C
7-1: 12794664A/T
7-1: 12794794G/A
7-1: 12794958C/T
7-1: 12795074T/A
7-1: 12795240A/T
7-1: 12796196T/G
7-1: 12796370A/G
7-1: 12796479A/T
7-1: 12798228G/A
7-1: 12799658T/C
7-1: 12801372C/G
7-1: 12814626T/C
7-1: 12826165G/T
7-1: 12847794G/A
7-1: 12865454T/C
7-1: 12952084T/C
7-1: 12955185G/T
7-1: 12976201A/G
7-1: 13011273C/A
7-1: 13122391T/A
7-1: 13134636G/A
7-1: 13167307C/A
7-1: 13225472T/C
7-1: 13282374T/A
7-1: 13308965T/A
7-1: 13339828C/A
7-1: 13400489C/T
7-1: 13464986T/C
7-1: 13480434T/A
7-1: 13485857C/T
7-1: 13485893T/A
7-1: 13493097C/G
7-1: 13493735C/A
7-1: 13513880A/G
7-1: 13558560T/C
7-1: 13695725G/A
7-1: 13745739G/A
7-1: 13794539A/G
7-1: 13890488G/A
7-1: 13904793T/A
7-1: 13937549C/G
7-1: 13964406G/C
7-1: 13982950G/A
7-1: 14020240T/C
7-1: 14029547T/G
7-1: 14137918A/T
7-1: 14162682G/T
7-1: 14238811G/C
7-1: 14261947T/A
7-1: 14390541C/T
7-1: 14444767G/A
7-1: 14482317A/G
7-1: 14555121G/C
7-1: 14693782T/C
7-1: 14704004A/G
7-1: 14705186T/C
7-1: 14737992G/A
7-1: 14855144A/G
7-1: 14870269A/G
7-1: 14961095G/A
7-1: 14971318C/T
7-1: 14978955C/T
7-1: 15106002A/G
7-1: 15300221T/A
7-1: 15417927G/A
7-1: 15478464G/A
7-1: 15558908C/A
7-1: 15570261G/A
7-1: 15607473T/C
7-1: 15661026C/T
7-1: 15683421G/T
7-1: 15738248T/C
7-1: 15823995C/T
7-1: 15825233C/T
7-1: 15861016T/C
7-1: 15893059C/T
7-1: 15968076C/G
7-1: 16076108T/C
7-1: 16165044A/G
7-1: 16183997T/C
7-1: 16216821T/A
7-1: 16243836G/T
7-1: 16342041A/G
7-1: 16448952G/A
7-1: 16487511A/G
7-1: 16515538G/A
7-1: 16547916T/C
7-1: 16562547A/G
7-1: 16686275C/T
7-1: 16726938C/A
7-1: 16819442A/G
7-1: 16832505T/G
7-1: 16842899G/A
7-1: 16867969T/C
7-1: 16968404G/T
7-1: 16982131A/C
7-1: 16993961A/T
7-1: 16995144A/G
7-1: 17097664A/T
7-1: 17120725G/T
7-1: 17144760A/C
7-1: 17148880C/T
7-1: 17152686T/C
7-1: 17166182A/G
7-1: 17193233T/C
7-1: 17214149A/G
7-1: 17234905T/C
7-1: 17253894A/G
7-1: 17287533C/A
7-1: 17532082G/A
7-1: 17539469T/C
7-1: 17557559T/C
7-1: 17616559C/T
7-1: 17643648A/C
7-1: 17683037G/A
7-1: 17736638C/G
7-1: 17750350C/T
7-1: 17796072G/A
7-1: 17853670G/A
7-1: 17883173A/G
7-1: 17915749C/T
7-1: 17949000A/G
7-1: 18101681G/A
7-1: 18111231G/A
7-1: 18112517G/A
7-1: 18137813G/A
7-1: 18190087C/T
7-1: 18232899A/C
7-1: 18245162A/T
7-1: 18265676G/A
7-1: 18282633A/T
7-1: 18398954G/A
7-1: 18504284A/T
7-1: 18508711A/G
7-1: 18542262A/C
7-1: 18551102C/T
7-1: 18563214G/T
7-1: 18588597T/C
7-1: 18608558C/T
7-1: 18635762A/T
7-1: 18653793C/T
7-1: 18665175C/T
7-1: 18674656C/T
7-1: 18676050T/A
7-1: 18687033C/T
7-1: 18692044T/C
7-1: 18705768A/C
7-1: 18709947C/T
7-1: 18747270C/T
7-1: 18816629T/G
7-1: 18839234C/T
7-1: 18850946T/C
7-1: 18851022C/T
7-1: 18904248T/G
7-1: 18908193T/A
7-1: 18932532A/G
7-1: 18985304C/G
7-1: 19016024C/T
7-1: 19038568G/A
7-1: 19044878G/T
7-1: 19055241C/T
7-1: 19070269C/A
7-1: 19102939G/A
7-1: 19134477A/C
7-1: 19145971T/C
7-1: 19158227A/G
7-1: 19293280G/C
7-1: 19385037C/T
7-1: 19461506C/T
7-1: 19507497C/T
7-1: 19581217T/G
7-1: 19630188T/C
7-1: 19699699G/A
7-1: 19701287G/A
7-1: 19701298T/C
7-1: 19971195T/A
7-1: 20003076A/G
7-1: 20095066C/T
7-1: 20133205A/G
7-1: 20202190G/A
7-1: 20257563G/A
7-1: 20274707C/G
7-1: 20300033C/T
7-1: 20313405G/T
7-1: 20314773C/T
7-1: 20373980A/G
7-1: 20411039T/C
7-1: 20418946T/.
7-1: 20422848A/T
7-1: 20436131C/T
7-1: 20507127A/C
7-1: 20658713A/C
7-1: 20672050C/T
7-1: 20681966G/A
7-1: 20702483A/G
7-1: 20759751T/A
7-1: 20822589A/G
7-1: 20823133C/T
7-1: 20823138T/G
7-1: 20839998C/G
7-1: 20842687A/C
7-1: 20842870A/T
7-1: 20842899G/T
7-1: 20842931T/C
7-1: 20843053C/T
7-1: 20852309A/G
7-1: 20863856T/A
7-1: 20864777C/T
7-1: 20865505C/G
7-1: 20865633A/G
7-1: 20866145T/C
7-1: 20866147C/T
7-1: 20866418G/T
7-1: 20880392C/T
7-1: 20892707A/G
7-1: 20959638C/A
7-1: 21015015C/A
7-1: 21031236A/G
7-1: 21057454G/A
7-1: 21118075C/T
7-1: 21134320A/T
7-1: 21245834T/A
7-1: 21248389G/A
7-1: 21266620G/A
7-1: 21310808G/A
7-1: 21380715A/G
7-1: 21484617C/A
7-1: 21630592C/T
7-1: 21640834C/T
7-1: 21803274T/C
7-1: 21952011C/T
7-1: 22104435T/G
7-1: 22184821A/G
7-1: 22290259A/C
7-1: 22317116T/C
7-1: 22317140C/T
7-1: 22332018C/T
7-1: 22344441C/T
7-1: 22359718T/C
7-1: 22388813G/A
7-1: 22433408C/T
7-1: 22445654A/T
7-1: 22460451G/A
7-1: 22460677C/T
7-1: 22487162T/A
7-1: 22531916C/A
7-1: 22538110C/T
7-1: 22586735C/T
7-1: 22615098T/A
7-1: 22650834T/C
7-1: 22687806C/T
7-1: 22750236G/T
7-1: 22766587C/T
7-1: 22783938A/C
7-1: 22794182T/C
7-1: 22795053T/C
7-1: 22795493A/T
7-1: 22800506A/T
7-1: 22800614A/T
7-1: 22800727A/C
7-1: 22802452C/T
7-1: 22804624G/T
7-1: 22817584C/T
7-1: 22825193A/G
7-1: 22836671T/C
7-1: 22846884A/G
7-1: 22871918A/C
7-1: 22949243G/T
7-1: 22959001C/T
7-1: 22959875C/A
7-1: 22975565C/A
7-1: 23017182A/G
7-1: 23076341G/A
7-1: 23156387T/A
7-1: 23185276T/C
7-1: 23185488T/C
7-1: 23187944A/G
7-1: 23189850C/T
7-1: 23190118A/G
7-1: 23192201G/C
7-1: 23194197A/T
7-1: 23195424C/T
7-1: 23204094T/C
7-1: 23298050G/A
7-1: 23349258G/A
7-1: 23398691A/G
7-1: 23413058C/A
7-1: 23463920A/G
7-1: 23546448C/T
7-1: 23598379A/T
7-1: 23606219C/T
7-1: 23606405T/A
7-1: 23607158C/T
7-1: 23611520T/A
7-1: 23643679C/T
7-1: 23661193C/G
7-1: 23783021C/A
7-1: 23794645A/C
7-1: 23796335T/C
7-1: 23825269C/T
7-1: 23837122G/A
7-1: 23875799A/C
7-1: 23931134C/T
7-1: 23974479C/G
7-1: 23989905C/T
7-1: 24042766G/C
7-1: 24049998T/C
7-1: 24054051C/A
7-1: 24054641A/G
7-1: 24061533T/A
7-1: 24071339C/T
7-1: 24096717T/C
7-1: 24112942C/T
7-1: 24115976G/A
7-1: 24116349T/A
7-1: 24116444T/A
7-1: 24119555C/T
7-1: 24184512A/T
7-1: 24233696C/T
7-1: 24250597A/C
7-1: 24306945A/G
7-1: 24315510C/G
7-1: 24325055T/C
7-1: 24416995C/T
7-1: 24512365G/A
7-1: 24526119G/A
7-1: 24561195C/T
7-1: 24625640C/A
7-1: 24741852A/C
7-1: 24797459A/G
7-1: 24840256G/A
7-1: 24974524G/A
7-1: 25041919T/A
7-1: 25083994T/G
7-1: 25123073C/T
7-1: 25137511C/G
7-1: 25156782C/T
7-1: 25225839T/C
7-1: 25239047C/T
7-1: 25252719A/G
7-1: 25285016A/T
7-1: 25313890C/.
7-1: 25360421G/C
7-1: 25360427A/T
7-1: 25425237G/T
7-1: 25509049C/T
7-1: 25580836G/A
7-1: 25631641T/A
7-1: 25822890G/T
7-1: 25961779T/A
7-1: 26052024T/C
7-1: 26066905A/T
7-1: 26077771C/T
7-1: 26175656C/A
7-1: 26179784T/C
7-1: 26195392C/A
7-1: 26195427C/T
7-1: 26203496G/A
7-1: 26206691C/T
7-1: 26216503T/C
7-1: 26216863C/A
7-1: 26229836T/G
7-1: 26257102G/A
7-1: 26271025G/C
7-1: 26320975C/T
7-1: 26376024C/T
7-1: 26413118G/T
7-1: 26441605T/A
7-1: 26461513T/C
7-1: 26472763C/T
7-1: 26501461G/A
7-1: 26532610A/G
7-1: 26550156G/A
7-1: 26561700T/G
7-1: 26576090C/T
7-1: 26576329C/T
7-1: 26577204G/A
7-1: 26579936T/C
7-1: 26604103T/G
7-1: 26604728C/A
7-1: 26616388C/G
7-1: 26651538T/G
7-1: 26667129T/C
7-1: 26682739G/A
7-1: 26709834G/A
7-1: 26779482A/G
7-1: 26923960T/G
7-1: 26953060T/G
7-1: 27005675C/A
7-1: 27128123A/G
7-1: 27177611A/G
7-1: 27189866G/A
7-1: 27200824C/G
7-1: 27256765A/T
7-1: 27270187G/A
7-1: 27272224T/C
7-1: 27285618T/C
7-1: 27311090C/G
7-1: 27393540T/C
7-1: 27435701T/A
7-1: 27442757T/A
7-1: 27442761C/T
7-1: 27445754C/A
7-1: 27498200T/A
7-1: 27499150C/T
7-1: 27515264G/T
7-1: 27526046G/A
7-1: 27528093C/T
7-1: 27592674A/G
7-1: 27593769C/T
7-1: 27615028G/A
7-1: 27642553T/A
7-1: 27680590T/G
7-1: 27682319C/T
7-1: 27684484A/T
7-1: 27691376C/T
7-1: 27699697G/A
7-1: 27700271G/A
7-1: 27710300C/A
7-1: 27710333C/T
7-1: 27710590G/A
7-1: 27740465G/C
7-1: 27781429G/T
7-1: 27789139G/A
7-1: 27789298A/G
7-1: 27789421T/C
7-1: 27793680C/A
7-1: 27997388A/C
7-1: 28074910A/G
7-1: 28086229T/C
7-1: 28086453C/T
7-1: 28087371G/A
7-1: 28087388C/T
7-1: 28098907G/T
7-1: 28099479C/T
7-1: 28099687C/T
7-1: 28100362C/T
7-1: 28100403C/T
7-1: 28100572G/A
7-1: 28100882G/A
7-1: 28100928G/A
7-1: 28101065G/T
7-1: 28101222G/A
7-1: 28101416A/C
7-1: 28101432G/A
7-1: 28101591T/C
7-1: 28101747A/T
7-1: 28101882G/C
7-1: 28101948G/A
7-1: 28102005T/C
7-1: 28102137G/A
7-1: 28102428A/T
7-1: 28102449T/A
7-1: 28102914G/T
7-1: 28103155C/T
7-1: 28103217T/G
7-1: 28103486T/C
7-1: 28103529T/C
7-1: 28106816G/A
7-1: 28107064A/G
7-1: 28107195G/T
7-1: 28107342G/A
7-1: 28108957A/G
7-1: 28109421C/T
7-1: 28109427T/A
7-1: 28141696T/C
7-1: 28145450T/C
7-1: 28268015G/A
7-1: 28355510T/C
7-1: 28357196G/A
7-1: 28482591G/A
7-1: 28505531A/G
7-1: 28519135G/A
7-1: 28529384A/G
7-1: 28582034G/C
7-1: 28600566A/T
7-1: 28669608T/C
7-1: 28674321C/A
7-1: 28736280A/G
7-1: 28769325G/C
7-1: 28799259C/T
7-1: 28838665T/C
7-1: 28944925A/G
7-1: 28960185G/T
7-1: 28970192T/G
7-1: 28975509A/G
7-1: 28994278A/G
7-1: 29063287G/A
7-1: 29713594T/G
7-1: 29716660C/T
7-1: 29727253T/A
7-1: 30049612G/A
7-1: 30130473A/G
7-1: 30142375A/T
7-1: 30196117T/G
7-1: 30356337G/A
7-1: 30525930A/T
7-1: 30535688A/G
7-1: 30552345T/A
7-1: 30624201A/G
7-1: 30707827A/C
7-1: 30747451T/C
7-1: 30850518C/T
7-1: 30861393C/G
7-1: 30861545A/G
7-1: 30898725C/T
7-1: 30908109C/T
7-1: 30908293T/A
7-1: 30912514T/C
7-1: 30924578G/A
7-1: 31083811T/C
7-1: 31138676G/A
7-1: 31190329A/G
7-1: 31267230C/G
7-1: 31303514C/A
7-1: 31328743A/T
7-1: 31361866A/G
7-1: 31399005A/G
7-1: 31508253A/T
7-1: 31577788A/G
7-1: 31601015A/T
7-1: 31603406A/G
7-1: 31643182T/A
7-1: 31676784A/T
7-1: 31706147C/T
7-1: 31714629A/C
7-1: 31716785T/C
7-1: 31732078G/A
7-1: 31746204G/A
7-1: 31762047G/A
7-1: 31779717A/G
7-1: 31793664T/C
7-1: 31803990G/A
7-1: 31826882T/A
7-1: 31882057C/T
7-1: 31897370A/G
7-1: 31916601T/G
7-1: 31961609C/T
7-1: 32010064A/G
7-1: 32152286A/G
7-1: 32185617A/G
7-1: 32200097C/T
7-1: 32210982G/T
7-1: 32249060C/T
7-1: 32262193T/G
7-1: 32295874C/A
7-1: 32308192T/C
7-1: 32310455A/G
7-1: 32325939A/G
7-1: 32367428T/A
7-1: 32384308G/A
7-1: 32400134G/A
7-1: 32461836T/C
7-1: 32472433T/A
7-1: 32489997T/A
7-1: 32519649C/T
7-1: 32529969G/C
7-1: 32571681T/C
7-1: 32582563A/C
7-1: 32602799A/G
7-1: 32624766A/C
7-1: 32697608T/C
7-1: 32710483C/T
7-1: 32724430C/T
7-1: 32764200G/A
7-1: 32785221A/T
7-1: 32799566A/G
7-1: 32813577T/A
7-1: 32824442C/T
7-1: 32834149T/A
7-1: 32834447T/C
7-1: 32834659C/A
7-1: 32834971C/T
7-1: 32835582C/T
7-1: 32888836T/A
7-1: 32890029A/G
7-1: 32890142T/A
7-1: 32900757A/C
7-1: 32912092G/A
7-1: 32923928T/C
7-1: 32970270C/T
7-1: 33016912G/A
7-1: 33051587C/T
7-1: 33072196T/C
7-1: 33083711A/G
7-1: 33114739C/G
7-1: 33125294G/T
7-1: 33130524T/C
7-1: 33130825T/C
7-1: 33131679A/G
7-1: 33131780C/G
7-1: 33131924G/T
7-1: 33132122T/A
7-1: 33132338C/T
7-1: 33132476T/C
7-1: 33132607C/G
7-1: 33132709G/A
7-1: 33135025T/C
7-1: 33135079C/G
7-1: 33135471G/T
7-1: 33135501T/C
7-1: 33136001T/G
7-1: 33136168A/G
7-1: 33136191T/G
7-1: 33136610T/C
7-1: 33136755A/G
7-1: 33137264C/A
7-1: 33137347T/C
7-1: 33137617C/G
7-1: 33208513C/T
7-1: 33404985T/G
7-1: 33421989C/G
7-1: 33424080C/T
7-1: 33527812T/C
7-1: 33545803A/G
7-1: 33549422A/G
7-1: 33564707G/A
7-1: 33566046G/A
7-1: 33568828C/T
7-1: 33568933A/G
7-1: 33582094G/A
7-1: 33592984G/T
7-1: 33616851G/A
7-1: 33632085C/A
7-1: 33637516T/C
7-1: 33642877A/G
7-1: 33694304A/T
7-1: 33724531A/G
7-1: 33750576A/C
7-1: 33805990T/C
7-1: 33867133T/C
7-1: 33892996T/C
7-1: 33899342T/C
7-1: 33916954T/C
7-1: 33943114G/C
7-1: 33999264C/A
7-1: 34036492T/C
7-1: 34040397G/T
7-1: 34048033C/T
7-1: 34048943T/A
7-1: 34049152C/T
7-1: 34053685T/C
7-1: 34060255C/T
7-1: 34071488C/T
7-1: 34093758T/C
7-1: 34176346G/A
7-1: 34200061A/C
7-1: 34232183G/A
7-1: 34264398T/C
7-1: 34292167C/T
7-1: 34307198C/A
7-1: 34339916G/T
7-1: 34361124T/G
7-1: 34379257C/T
7-1: 34429405G/A
7-1: 34439935G/A
7-1: 34457052C/T
7-1: 34529699G/A
7-1: 34541455G/A
7-1: 34561356G/A
7-1: 34572542C/T
7-1: 34602999C/T
7-1: 34624182C/A
7-1: 34739823G/A
7-1: 34818927T/C
7-1: 34830092C/T
7-1: 34866637C/T
7-1: 34872207G/A
7-1: 34878953C/A
7-1: 34934080T/G
7-1: 34949496A/G
7-1: 34967768A/T
7-1: 34978504C/A
7-1: 34980807C/A
7-1: 34990076T/G
7-1: 35007272C/G
7-1: 35012844C/T
7-1: 35016029T/G
7-1: 35020217G/A
7-1: 35047706T/C
7-1: 35050621A/C
7-1: 35074129A/G
7-1: 35088175C/G
7-1: 35108494T/C
7-1: 35113867G/A
7-1: 35174451C/T
7-1: 35193068T/A
7-1: 35309628A/T
7-1: 35322560A/C
7-1: 35322679G/A
7-1: 35323022A/G
7-1: 35323123A/T
7-1: 35323771T/C
7-1: 35327661C/T
7-1: 35329403T/C
7-1: 35331054T/A
7-1: 35331158G/A
7-1: 35331268A/G
7-1: 35335113T/C
7-1: 35363826G/T
7-1: 35446759T/C
7-1: 35499984C/T
7-1: 35541296A/G
7-1: 35588779T/A
7-1: 35735170G/A
7-1: 35749441C/T
7-1: 35755486G/A
7-1: 35783005T/C
7-1: 35855453C/T
7-1: 35958937A/T
7-1: 36192648T/C
7-1: 36235784A/G
7-1: 36332575C/T
7-1: 36386841T/C
7-1: 36394567T/C
7-1: 36403749C/A
7-1: 36415195C/T
7-1: 36484978A/G
7-1: 36558050A/G
7-1: 36564941T/A
7-1: 36564944A/T
7-1: 36564994C/A
7-1: 36565097G/A
7-1: 36565137A/T
7-1: 36565145T/C
7-1: 36565179G/A
7-1: 36565263G/A
7-1: 36565333C/A
7-1: 36650650C/A
7-1: 36745328T/C
7-1: 36793319G/A
7-1: 36805590C/T
7-1: 36821574T/A
7-1: 36847428C/T
7-1: 36847822G/A
7-1: 36896609G/C
7-1: 36948753C/T
7-1: 36988277C/T
7-1: 37010981G/A
7-1: 37018298A/C
7-1: 37036743G/A
7-1: 37063811G/A
7-1: 37076841A/C
7-1: 37090977A/G
7-1: 37118848A/G
7-1: 37129788T/C
7-1: 37167785A/G
7-1: 37184767C/T
7-1: 37230566A/G
7-1: 37308799A/C
7-1: 37383736G/A
7-1: 37432701T/A
7-1: 37510025T/C
7-1: 37524041G/T
7-1: 37548425G/T
7-1: 37575369C/A
7-1: 37627852G/A
7-1: 37638808G/A
7-1: 37657462T/A
7-1: 37660463G/T
7-1: 37660476T/C
7-1: 37661028T/A
7-1: 37661382G/T
7-1: 37661536G/A
7-1: 37662128A/T
7-1: 37662225G/A
7-1: 37692248G/A
7-1: 37725830T/C
7-1: 37749908C/T
7-1: 37766084C/T
7-1: 37805068G/A
7-1: 37821281A/G
7-1: 37824630A/G
7-1: 37827799G/T
7-1: 37828160C/T
7-1: 37829019G/A
7-1: 37829259G/A
7-1: 37829604C/T
7-1: 37831663A/C
7-1: 37835634A/G
7-1: 37835705G/C
7-1: 37835807G/A
7-1: 37839741T/A
7-1: 37839747C/T
7-1: 37844555G/A
7-1: 37845465C/T
7-1: 37848996T/C
7-1: 37849071G/T
7-1: 37849144G/A
7-1: 37849969T/A
7-1: 37850126T/A
7-1: 37850348A/C
7-1: 37850459G/A
7-1: 37850659C/T
7-1: 37854897G/A
7-1: 37878502G/A
7-1: 37908251C/T
7-1: 37912705A/G
7-1: 37912870C/G
7-1: 37918365G/C
7-1: 37919766C/T
7-1: 37928700C/A
7-1: 37928849T/A
7-1: 37929098G/A
7-1: 37934327C/T
7-1: 37934354C/A
7-1: 37935658T/A
7-1: 37936124G/A
7-1: 37936489C/G
7-1: 37937666A/C
7-1: 37937775G/T
7-1: 37938027T/A
7-1: 37938802A/C
7-1: 37938877G/A
7-1: 37938888C/T
7-1: 37948720C/T
7-1: 37948899C/A
7-1: 37949174A/C
7-1: 37949576C/G
7-1: 37949771T/G
7-1: 37950080A/T
7-1: 37952080T/C
7-1: 37954090C/G
7-1: 37954227C/T
7-1: 37954252G/A
7-1: 37954265T/G
7-1: 37954482G/A
7-1: 37970641A/C
7-1: 37982904T/C
7-1: 38004846A/G
7-1: 38023317T/C
7-1: 38036059C/T
7-1: 38048877C/T
7-1: 38089780T/A
7-1: 38112342G/A
7-1: 38112632G/T
7-1: 38113097G/T
7-1: 38113606T/A
7-1: 38134173A/G
7-1: 38135450T/C
7-1: 38136018C/A
7-1: 38150376C/T
7-1: 38259553G/A
7-1: 38270014G/T
7-1: 38285212G/A
7-1: 38286251C/G
7-1: 38295717C/T
7-1: 38316340T/A
7-1: 38331324G/A
7-1: 38350293A/G
7-1: 38355692G/A
7-1: 38356193A/G
7-1: 38356339C/T
7-1: 38358077T/A
7-1: 38358886G/T
7-1: 38375102C/T
7-1: 38389072G/T
7-1: 38389123T/A
7-1: 38389550A/T
7-1: 38389592G/A
7-1: 38391005A/G
7-1: 38391227T/C
7-1: 38392561G/A
7-1: 38407948T/C
7-1: 38423580A/G
7-1: 38436044C/T
7-1: 38436057C/T
7-1: 38436180C/T
7-1: 38436879A/T
7-1: 38445791T/A
7-1: 38445856A/G
7-1: 38447122G/A
7-1: 38450045C/T
7-1: 38450776T/C
7-1: 38520863A/T
7-1: 38534955A/G
7-1: 38552771A/G
7-1: 38580354G/T
7-1: 38594542C/T
7-1: 38657886C/G
7-1: 38725789G/T
7-1: 38757628G/C
7-1: 38821631C/A
7-1: 38833885T/C
7-1: 38857666T/A
7-1: 38857806A/T
7-1: 38857868A/T
7-1: 38857923T/A
7-1: 38858275A/T
7-1: 38868239C/T
7-1: 38937509G/A
7-1: 38968662A/G
7-1: 38987914A/G
7-1: 39029975C/T
7-1: 39033270T/A
7-1: 39035040T/C
7-1: 39037026A/C
7-1: 39037163A/C
7-1: 39046439C/T
7-1: 39089247T/A
7-1: 39105906A/G
7-1: 39129423A/T
7-1: 39141483A/G
7-1: 39146248T/C
7-1: 39146874T/C
7-1: 39147637G/A
7-1: 39147740G/A
7-1: 39150636C/T
7-1: 39152598A/T
7-1: 39153472A/T
7-1: 39169366G/A
7-1: 39181853G/A
7-1: 39213307A/G
7-1: 39242602C/A
7-1: 39306204G/A
7-1: 39338883T/C
7-1: 39350106A/T
7-1: 39361491T/C
7-1: 39373866T/G
7-1: 39375844A/G
7-1: 39383636T/C
7-1: 39388040C/G
7-1: 39414327T/A
7-1: 39474893C/T
7-1: 39525320T/G
7-1: 39526707A/T
7-1: 39526720G/A
7-1: 39527032C/A
7-1: 39527103G/T
7-1: 39537284T/A
7-1: 39552237G/T
7-1: 39572539T/C
7-1: 39587136T/C
7-1: 39598794G/A
7-1: 39621729T/A
7-1: 39677690A/C
7-1: 39769701A/T
7-1: 39771916C/A
7-1: 39784117T/A
7-1: 39786543C/A
7-1: 39788496C/A
7-1: 39794508T/C
7-1: 39805436G/A
7-1: 39828960T/A
7-1: 39839709G/T
7-1: 39850471A/C
7-1: 39867669A/G
7-1: 39884838G/A
7-1: 39913740A/T
7-1: 39927583C/T
7-1: 39930926C/A
7-1: 39959146G/A
7-1: 40073055G/T
7-1: 40113184C/T
7-1: 40145856G/A
7-1: 40183176G/T
7-1: 40213153C/G
7-1: 40232687A/G
7-1: 40232844C/A
7-1: 40235541G/A
7-1: 40246274C/T
7-1: 40248625A/G
7-1: 40263459G/T
7-1: 40267824A/T
7-1: 40271911C/T
7-1: 40342878T/C
7-1: 40369568G/A
7-1: 40383295A/C
7-1: 40430559G/A
7-1: 40459976G/T
7-1: 40478208G/T
7-1: 40492990A/G
7-1: 40504282A/C
7-1: 40618170T/C
7-1: 40720900G/A
7-1: 40722300T/G
7-1: 40733477G/A
7-1: 40831002T/G
7-1: 40853753C/G
7-1: 40979578A/G
7-1: 40996633C/T
7-1: 41015806T/C
7-1: 41022001G/T
7-1: 41061517A/G
7-1: 41061762G/A
7-1: 41064093G/A
7-1: 41064326T/A
7-1: 41075904C/T
7-1: 41086189T/C
7-1: 41112173T/C
7-1: 41123287T/C
7-1: 41241707T/G
7-1: 41245306T/C
7-1: 41257951A/C
7-1: 41457592A/G
7-1: 41569580C/T
7-1: 41592738G/C
7-1: 41610250C/A
7-1: 41624098A/C
7-1: 41640416C/T
7-1: 41842367T/C
7-1: 41864775G/A
7-1: 41874801C/A
7-1: 41894047C/A
7-1: 41933853C/A
7-1: 41981294T/A
7-1: 42161534C/T
7-1: 42167557A/G
7-1: 42179489A/G
7-1: 42191796A/G
7-1: 42191864A/G
7-1: 42245301A/G
7-1: 42389544A/T
7-1: 42442119T/G
7-1: 42770843A/G
7-1: 42776099C/T
7-1: 42789893A/C
7-1: 42803427G/A
7-1: 42826024C/T
7-1: 42840481A/G
7-1: 42851624C/A
7-1: 42860830G/A
7-1: 42900716A/C
7-1: 42948161A/G
7-1: 42990751T/A
7-1: 43025608T/A
7-1: 43056454G/A
7-1: 43145642C/T
7-1: 43158498A/C
7-1: 43336493C/A
7-1: 43641824C/T
7-1: 43669268T/C
7-1: 43931466T/A
7-1: 43937241T/C
7-1: 43956650C/T
7-1: 43967379T/C
7-1: 44103449G/A
7-1: 44182994G/A
7-1: 44185414C/T
7-1: 44185426A/G
7-1: 44188280G/C
7-1: 44188359A/G
7-1: 44189719T/C
7-1: 44199596A/C
7-1: 44225603T/A
7-1: 44433865C/A
7-1: 44435238A/T
7-1: 44436083G/T
7-1: 44472215G/A
7-1: 44564470C/G
7-1: 44620425C/A
7-1: 44676835G/A
7-1: 44677639C/T
7-1: 44677673A/G
7-1: 44698701A/G
7-1: 44795915C/G
7-1: 44819951G/A
7-1: 44832401A/T
7-1: 44842787A/C
7-1: 44849370C/G
7-1: 44939236T/A
7-1: 45014223C/T
7-1: 45015897C/T
7-1: 45016120A/T
7-1: 45019561G/T
7-1: 45019720T/G
7-1: 45019731T/A
7-1: 45030015A/G
7-1: 45055025C/G
7-1: 45078919A/G
7-1: 45102249G/A
7-1: 45119525G/T
7-1: 45132765T/C
7-1: 45231703C/A
7-1: 45254853G/T
7-1: 45254857C/A
7-1: 45255527A/G
7-1: 45255703A/G
7-1: 45268877A/T
7-1: 45287703T/A
7-1: 45294606C/T
7-1: 45298135G/T
7-1: 45299722A/G
7-1: 45306712T/C
7-1: 45306827G/A
7-1: 45307133G/A
7-1: 45307299C/G
7-1: 45307404G/C
7-1: 45307481C/T
7-1: 45307515A/T
7-1: 45324352A/T
7-1: 45335699T/C
7-1: 45351831T/A
7-1: 45358293T/C
7-1: 45358302C/T
7-1: 45358321C/T
7-1: 45358404C/T
7-1: 45366921T/A
7-1: 45377306A/T
7-1: 45450606G/A
7-1: 45510774C/T
7-1: 45535001C/A
7-1: 45555496T/A
7-1: 45574833C/G
7-1: 45587516T/C
7-1: 45600015C/G
7-1: 45618479A/T
7-1: 45683684C/T
7-1: 45695970A/T
7-1: 45708778G/T
7-1: 45719414C/T
7-1: 45720279G/A
7-1: 45722731A/G
7-1: 45723067T/G
7-1: 45723083A/C
7-1: 45742154G/C
7-1: 45870758A/G
7-1: 45880890G/T
7-1: 45893774T/C
7-1: 45961627C/T
7-1: 45978350C/A
7-1: 46013201A/C
7-1: 46045328T/A
7-1: 46090480A/C
7-1: 46113538A/T
7-1: 46130443G/C
7-1: 46153230A/T
7-1: 46165787A/G
7-1: 46207701C/T
7-1: 46235391T/G
7-1: 46262255T/C
7-1: 46309930T/C
7-1: 46320468G/A
7-1: 46359280C/G
7-1: 46369832T/C
7-1: 46410267G/C
7-1: 46481605T/A
7-1: 46546566C/T
7-1: 46587346C/A
7-1: 46623437T/C
7-1: 46635633T/A
7-1: 46635672T/C
7-1: 46649611C/G
7-1: 46662049A/G
7-1: 46681034C/A
7-1: 46733654T/C
7-1: 46742562G/A
7-1: 46742714C/T
7-1: 46743050G/T
7-1: 46743064T/A
7-1: 46743936A/G
7-1: 46761765G/A
7-1: 46762411T/A
7-1: 46764587A/T
7-1: 46768803T/A
7-1: 46778868T/G
7-1: 46791502C/T
7-1: 46822163C/A
7-1: 46843053T/A
7-1: 46853323G/A
7-1: 46863389C/A
7-1: 46888559T/C
7-1: 46899984T/C
7-1: 46904266A/G
7-1: 46904895T/C
7-1: 46917073G/A
7-1: 46944208T/A
7-1: 47043402G/T
7-1: 47122596G/A
7-1: 47238458G/A
7-1: 47253341C/G
7-1: 47274979G/A
7-1: 47275021A/G
7-1: 47297909C/T
7-1: 47323872A/G
7-1: 47385984C/T
7-1: 47430610G/C
7-1: 47466799A/C
7-1: 47467136T/C
7-1: 47647249G/A
7-1: 47648298C/T
7-1: 47714776T/C
7-1: 47789941A/T
7-1: 47933832A/G
7-1: 47952714C/A
7-1: 47980123T/A
7-1: 47992811C/T
7-1: 47999792T/C
7-1: 47999895T/G
7-1: 48000170A/G
7-1: 48000458G/T
7-1: 48015932T/C
7-1: 48027493C/T
7-1: 48042614T/C
7-1: 48203917C/T
7-1: 48263266C/G
7-1: 48298924A/G
7-1: 48333396T/C
7-1: 48392239A/T
7-1: 48431912A/G
7-1: 48467637G/A
7-1: 48470154A/T
7-1: 48471491A/G
7-1: 48472047C/A
7-1: 48475782A/C
7-1: 48475829G/T
7-1: 48475833G/T
7-1: 48475887C/T
7-1: 48492581A/G
7-1: 48496170A/C
7-1: 48497214G/T
7-1: 48526424A/G
7-1: 48597451G/T
7-1: 48639363G/C
7-1: 48670929T/C
7-1: 48700413G/A
7-1: 48790377G/C
7-1: 48856282C/A
7-1: 48878550A/T
7-1: 48995760C/T
7-1: 49030363G/C
7-1: 49108023C/T
7-1: 49141949C/G
7-1: 49188002C/T
7-1: 49228543A/T
7-1: 49283999A/G
7-1: 49301145C/T
7-1: 49348061C/A
7-1: 49375159A/G
7-1: 49413650C/T
7-1: 49471221G/A
7-1: 49496364G/C
7-1: 49527814T/C
7-1: 49557070A/G
7-1: 49628936C/T
7-1: 49718220C/T
7-1: 49732418A/G
7-1: 49780809T/G
7-1: 49832536T/C
7-1: 49850616G/T
7-1: 49865382T/A
7-1: 49866337G/A
7-1: 49870797G/A
7-1: 49881339T/C
7-1: 49903022C/T
7-1: 49919351G/A
7-1: 49981732A/G
7-1: 50034372C/A
7-1: 50049352C/T
7-1: 50087266G/A
7-1: 50114925G/C
7-1: 50167763G/C
7-1: 50216605A/G
7-1: 50271445C/T
7-1: 50322684C/T
7-1: 50381551T/G
7-1: 50416409T/G
7-1: 50443279C/T
7-1: 50456176C/A
7-1: 50494793T/A
7-1: 50504881T/C
7-1: 50505012C/T
7-1: 50505193T/C
7-1: 50514858T/C
7-1: 50514912T/A
7-1: 50548022C/A
7-1: 50577937T/G
7-1: 50612726T/A
7-1: 50625859T/C
7-1: 50627378C/T
7-1: 50636001A/T
7-1: 50636131T/C
7-1: 50683855T/C
7-1: 50727869A/C
7-1: 50813425C/T
7-1: 50969974C/T
7-1: 51008369A/T
7-1: 51020495G/C
7-1: 51047663T/C
7-1: 51082571G/T
7-1: 51130480C/A
7-1: 51130512G/A
7-1: 51211463G/A
7-1: 51251601T/C
7-1: 51400834C/A
7-1: 51415847T/C
7-1: 51428051T/C
7-1: 51428221A/T
7-1: 51428620T/A
7-1: 51432755G/A
7-1: 51433005G/T
7-1: 51585903G/T
7-1: 51620621A/G
7-1: 51627501T/C
7-1: 51638598A/G
7-1: 51669358A/G
7-1: 51692982T/A
7-1: 51697771T/G
7-1: 51698789C/T
7-1: 51709271G/C
7-1: 51799788C/T
7-1: 51882385G/T
7-1: 51889617T/C
7-1: 51890287C/T
7-1: 51946618G/A
7-1: 51955216T/A
7-1: 52003158C/A
7-1: 52003184G/A
7-1: 52005314A/G
7-1: 52007280A/G
7-1: 52007282C/G
7-1: 52012453T/C
7-1: 52013073C/A
7-1: 52068515T/C
7-1: 52069697G/A
7-1: 52071360T/C
7-1: 52071914T/G
7-1: 52071918A/G
7-1: 52072030C/T
7-1: 52072201A/T
7-1: 52072208G/A
7-1: 52072420G/A
7-1: 52074012G/A
7-1: 52074080A/G
7-1: 52075296C/T
7-1: 52084440T/G
7-1: 52139804G/C
7-1: 52281449G/A
7-1: 52299165A/T
7-1: 52309518A/T
7-1: 52376951T/C
7-1: 52387623T/A
7-1: 52594684A/C
7-1: 52656392G/A
7-1: 52723741T/G
7-1: 52777383G/C
7-1: 52859999A/T
7-1: 52965549G/A
7-1: 53202438C/A
7-1: 53294126C/T
7-1: 53475577C/T
7-1: 53565518T/C
7-1: 53613979T/C
7-1: 53744414T/C
7-1: 53756551G/A
7-1: 53780490C/T
7-1: 53793757A/G
7-1: 53852697A/G
7-1: 53889220G/A
7-1: 53910792T/G
7-1: 53922390A/G
7-1: 53927849A/T
7-1: 53958175G/A
7-1: 53988674A/G
7-1: 54009103C/T
7-1: 54025353G/A
7-1: 54044142C/A
7-1: 54063036G/T
7-1: 54067004G/T
7-1: 54111052C/T
7-1: 54153106T/A
7-1: 54182959C/T
7-1: 54261601G/A
7-1: 54387531G/A
7-1: 54402158C/T
7-1: 54428728T/G
7-1: 54468450C/A
7-1: 54512822G/A
7-1: 54527601A/T
7-1: 54537959T/C
7-1: 54539194G/A
7-1: 54539351G/A
7-1: 54554231T/G
7-1: 54562057A/T
7-1: 54572610C/A
7-1: 54629937G/A
7-1: 54734548G/A
7-1: 54778168C/T
7-1: 54778269T/A
7-1: 54778551C/G
7-1: 54778591T/A
7-1: 54778845C/A
7-1: 54800287A/T
7-1: 54812688A/T
7-1: 54814063C/T
7-1: 54865931A/T
7-1: 55014202T/A
7-1: 55025891A/G
7-1: 55025996G/A
7-1: 55026570A/G
7-1: 55026719G/A
7-1: 55026728T/C
7-1: 55027716T/A
7-1: 55043164C/T
7-1: 55056265C/T
7-1: 55095382A/G
7-1: 55122365A/T
7-1: 55143742G/A
7-1: 55160129A/G
7-1: 55191352T/G
7-1: 55310037T/C
7-1: 55350983G/A
7-1: 55402525C/A
7-1: 55454996G/T
7-1: 55480613G/A
7-1: 55546133A/G
7-1: 55557442G/T
7-1: 55628741T/A
7-1: 55718234T/A
7-1: 55719445T/C
7-1: 55762671A/G
7-1: 55812090G/T
7-1: 55813187C/A
7-1: 56002767A/G
7-1: 56009648T/C
7-1: 56021793T/C
7-1: 56033538G/A
7-1: 56147113C/A
7-1: 56151619G/A
7-1: 56274046T/A
7-1: 56318905A/C
7-1: 56335271A/T
7-1: 56349455G/A
7-1: 56443841A/G
7-1: 56486779G/A
7-1: 56613631C/T
7-1: 56615235A/G
7-1: 56615346C/T
7-1: 56635575T/C
7-1: 56777656G/C
7-1: 56777848A/G
7-1: 56778128A/C
7-1: 56812469C/A
7-1: 56973865G/A
7-1: 57145679A/T
7-1: 57171149A/T
7-1: 57210182A/G
7-1: 57228805G/C
7-1: 57238609G/A
7-1: 57301581A/G
7-1: 57353824A/C
7-1: 57379081A/G
7-1: 57383737C/T
7-1: 57387467A/C
7-1: 57387698G/A
7-1: 57390979A/T
7-1: 57391029T/G
7-1: 57464953A/G
7-1: 57535134G/A
7-1: 57539781A/T
7-1: 57602020T/A
7-1: 57630185C/T
7-1: 57677838T/C
7-1: 57697213T/A
7-1: 57711114T/A
7-1: 57712794G/A
7-1: 57712795C/A
7-1: 57736610G/A
7-1: 57792754T/A
7-1: 57792782C/G
7-1: 57798917T/C
7-1: 57843595C/T
7-1: 57845258A/G
7-1: 57874213A/G
7-1: 57938150T/A
7-1: 57971605T/C
7-1: 58057820T/C
7-1: 58084082G/A
7-1: 58117861G/A
7-1: 58127909G/A
7-1: 58148074T/G
7-1: 58205545C/T
7-1: 58314522C/A
7-1: 58407036A/G
7-1: 58474107C/T
7-1: 58479058C/T
7-1: 58519373C/T
7-1: 58525824G/A
7-1: 58538782T/C
7-1: 58555050T/A
7-1: 58574285G/T
7-1: 58584662G/A
7-1: 58694624A/G
7-1: 58696993C/T
7-1: 58723512G/A
7-1: 58742866A/T
7-1: 58802730C/T
7-1: 58877906T/C
7-1: 58916790G/A
7-1: 59081511T/C
7-1: 59115798C/A
7-1: 59140330T/A
7-1: 59169568C/G
7-1: 59201155C/T
7-1: 59231261G/A
7-1: 59232349T/C
7-1: 59263905C/T
7-1: 59306328T/C
7-1: 59349239T/A
7-1: 59384374C/T
7-1: 59446121G/T
7-1: 59517511C/T
7-1: 59640713G/T
7-1: 59856268G/A
7-1: 59861838C/G
7-1: 59905266G/A
7-1: 59928060C/T
7-1: 60007983C/T
7-1: 60057166A/G
7-1: 60105103T/A
7-1: 60127225G/A
7-1: 60139289T/C
7-1: 60155567G/A
7-1: 60166646G/A
7-1: 60167604G/A
7-1: 60224192T/A
7-1: 60242959T/C
7-1: 60257394T/C
7-1: 60268794G/A
7-1: 60285393A/G
7-1: 60314886C/T
7-1: 60370735C/T
7-1: 60378133A/G
7-1: 60392855T/A
7-1: 60397129G/A
7-1: 60430313T/A
7-1: 60434711T/C
7-1: 60435482T/G
7-1: 60435593G/T
7-1: 60448551T/G
7-1: 60558931T/A
7-1: 60571543A/T
7-1: 60571987T/C
7-1: 60589327T/A
7-1: 60672827C/T
7-1: 60734888C/T
7-1: 60735050G/A
7-1: 60735188A/C
7-1: 60735339A/G
7-1: 60736219T/A
7-1: 60736249T/C
7-1: 60736646G/A
7-1: 60736750T/A
7-1: 60736876A/G
7-1: 60737122G/C
7-1: 60737675C/G
7-1: 60737707C/T
7-1: 60796141G/A
7-1: 60861185G/A
7-1: 60898160T/C
7-1: 60918466T/A
7-1: 60965856T/C
7-1: 61006061T/C
7-1: 61016256T/C
7-1: 61016589A/G
7-1: 61016863C/T
7-1: 61017044A/G
7-1: 61017050C/A
7-1: 61019299A/G
7-1: 61083613T/C
7-1: 61126802T/C
7-1: 61250900A/T
7-1: 61288735T/G
7-1: 61307229A/G
7-1: 61316940A/G
7-1: 61317307T/C
7-1: 61317502C/T
7-1: 61317547G/A
7-1: 61317643G/A
7-1: 61317695T/A
7-1: 61317982C/T
7-1: 61318202A/G
7-1: 61318283C/A
7-1: 61318391T/A
7-1: 61318512C/T
7-1: 61327309A/T
7-1: 61327424C/T
7-1: 61407177C/T
7-1: 61455901A/T
7-1: 61515551T/C
7-1: 61607958G/A
7-1: 61638535C/T
7-1: 61648568C/A
7-1: 61659439A/T
7-1: 61670993A/G
7-1: 61689590T/G
7-1: 61702053G/A
7-1: 61722482A/G
7-1: 61761250C/T
7-1: 61805821A/T
7-1: 61808857G/A
7-1: 61912407A/G
7-1: 61961742G/A
7-1: 62012072A/G
7-1: 62020859A/C
7-1: 62021221A/T
7-1: 62021398G/T
7-1: 62021407T/A
7-1: 62025862C/G
7-1: 62044835G/A
7-1: 62187915C/G
7-1: 62237308T/C
7-1: 62265000G/A
7-1: 62297015C/A
7-1: 62364254T/G
7-1: 62410797C/G
7-1: 62461156G/A
7-1: 62475249A/G
7-1: 62587122G/A
7-1: 62694967T/C
7-1: 62696280G/T
7-1: 62724605C/T
7-1: 62775119C/G
7-1: 62827123G/A
7-1: 62878467A/C
7-1: 62879467A/T
7-1: 62943133C/A
7-1: 62948972T/C
7-1: 62984528A/G
7-1: 63070577C/T
7-1: 63074662T/A
7-1: 63076758G/A
7-1: 63148687C/T
7-1: 63177969G/A
7-1: 63221126T/G
7-1: 63242477T/C
7-1: 63391143A/T
7-1: 63391354C/T
7-1: 63461211T/G
7-1: 63474938C/A
7-1: 63488347G/A
7-1: 63506226G/T
7-1: 63512696A/T
7-1: 63564962A/T
7-1: 63565179C/A
7-1: 63565244A/T
7-1: 63614726T/A
7-1: 63660818G/A
7-1: 63713720A/T
7-1: 63771867A/C
7-1: 63840981T/C
7-1: 63890369C/G
7-1: 63920707T/G
7-1: 63943381C/T
7-1: 63980872G/A
7-1: 63993490G/A
7-1: 64074294A/G
7-1: 64139539A/G
7-1: 64140090C/T
7-1: 64140228A/T
7-1: 64180293C/T
7-1: 64194995T/G
7-1: 64205528T/G
7-1: 64261948A/T
7-1: 64297634C/A
7-1: 64376875A/T
7-1: 64410939C/A
7-1: 64457397G/A
7-1: 64491967G/A
7-1: 64589976C/T
7-1: 64614325T/C
7-1: 64656572C/T
7-1: 64713988T/G
7-1: 64771689A/G
7-1: 64791530C/T
7-1: 64807528C/T
7-1: 64855086T/C
7-1: 64865756T/A
7-1: 64942911A/G
7-1: 65016712G/A
7-1: 65076602G/A
7-1: 65142013G/A
7-1: 65146110T/C
7-1: 65189096C/T
7-1: 65381595C/T
7-1: 65445105C/T
7-1: 65493637A/G
7-1: 65493658G/A
7-1: 65586497C/T
7-1: 65608871A/T
7-1: 65609450G/.
7-1: 65637486A/G
7-1: 65654968C/A
7-1: 65687324A/T
7-1: 65730424C/T
7-1: 65782529T/A
7-1: 65798958A/G
7-1: 65821359A/C
7-1: 65834174G/.
7-1: 65834424A/.
7-1: 65834449T/C
7-1: 65834521C/T
7-1: 65834596A/G
7-1: 65834632A/G
7-1: 65835137T/C
7-1: 65835632G/A
7-1: 65836163A/T
7-1: 65836269C/T
7-1: 65859255A/T
7-1: 65935550C/T
7-1: 65984050A/G
7-1: 65984206A/T
7-1: 66025101G/C
7-1: 66101709C/T
7-1: 66139115A/G
7-1: 66180050G/T
7-1: 66232599T/A
7-1: 66249044C/T
7-1: 66360411T/C
7-1: 66378794G/A
7-1: 66439353G/T
7-1: 66462008C/A
7-1: 66485474A/C
7-1: 66510647A/C
7-1: 66532994G/A
7-1: 66545041A/G
7-1: 66583937T/C
7-1: 66615276T/A
7-1: 66622907T/A
7-1: 66633886T/C
7-1: 66671239C/A
7-1: 66745155G/A
7-1: 66793350C/G
7-1: 66805158A/C
7-1: 66819075G/A
7-1: 66845263C/T
7-1: 66857313G/A
7-1: 66867911A/G
7-1: 66918962T/C
7-1: 66928991G/C
7-1: 66929643T/A
7-1: 66936023A/G
7-1: 66946120G/C
7-1: 66963677G/T
7-1: 66972136T/C
7-1: 66972312T/C
7-1: 66974369G/T
7-1: 66994177T/A
7-1: 67001855C/T
7-1: 67030652T/C
7-1: 67060851A/G
7-1: 67074569T/C
7-1: 67120446C/T
7-1: 67135793C/T
7-1: 67152237A/G
7-1: 67182135C/A
7-1: 67220612T/G
7-1: 67233409G/A
7-1: 67245676T/C
7-1: 67257915C/T
7-1: 67271440G/T
7-1: 67307908T/C
7-1: 67310876A/G
7-1: 67326516A/G
7-1: 67331685G/A
7-1: 67333402C/A
7-1: 67343640T/C
7-1: 67378377G/A
7-1: 67406632T/C
7-1: 67422107G/A
7-1: 67525195G/T
7-1: 67585459A/G
7-1: 67606996A/G
7-1: 67657560G/A
7-1: 67741439A/C
7-1: 67854745A/T
7-1: 67857182T/C
7-1: 67908893C/A
7-1: 68023688A/C
7-1: 68035693C/A
7-1: 68059476A/T
7-1: 68091573T/A
7-1: 68131584C/T
7-1: 68164469A/C
7-1: 68213700T/A
7-1: 68277852G/C
7-1: 68320163A/C
7-1: 68356940C/T
7-1: 68388526G/A
7-1: 68406513G/T
7-1: 68411815T/A
7-1: 68411962T/C
7-1: 68411974T/C
7-1: 68677840A/T
7-1: 68687099T/C
7-1: 68716177G/A
7-1: 68725613C/T
7-1: 68762376A/G
7-1: 68796537T/C
7-1: 68826594C/T
7-1: 68870194T/C
7-1: 68931310A/G
7-1: 68965975T/G
7-1: 69020814T/C
7-1: 69064850C/T
7-1: 69108034T/C
7-1: 69209318T/C
7-1: 69292891C/T
7-1: 69391453C/T
7-1: 69446550G/A
7-1: 69455132A/G
7-1: 69464447C/T
7-1: 69509535A/T
7-1: 69550251C/A
7-1: 69564100C/A
7-1: 69589570C/T
7-1: 69638340T/C
7-1: 69695892A/G
7-1: 69742013C/A
7-1: 69789210G/A
7-1: 69832389A/T
7-1: 69871508G/A
7-1: 70039617C/A
7-1: 70039658A/C
7-1: 70039662G/A
7-1: 70039701C/T
7-1: 70039707C/T
7-1: 70040392T/A
7-1: 70050764T/G
7-1: 70142907G/A
7-1: 70153589C/G
7-1: 70166084C/A
7-1: 70189099A/G
7-1: 70200745A/G
7-1: 70217034G/C
7-1: 70236116C/A
7-1: 70254911T/A
7-1: 70285434G/A
7-1: 70348302C/T
7-1: 70383796A/G
7-1: 70481473A/G
7-1: 70562111C/A
7-1: 70595529A/G
7-1: 70664333C/T
7-1: 70693408T/A
7-1: 70722685T/A
7-1: 70747806C/A
7-1: 70747818G/A
7-1: 70772334T/C
7-1: 70800560G/A
7-1: 70804653A/C
7-1: 70804814C/T
7-1: 70813011G/A
7-1: 70816657C/A
7-1: 70816951A/C
7-1: 70823911C/T
7-1: 70829930C/T
7-1: 70836583C/T
7-1: 70847864G/C
7-1: 70847949G/A
7-1: 70898078A/T
7-1: 70928972A/T
7-1: 70944086A/G
7-1: 70974371A/G
7-1: 71014114A/G
7-1: 71027856G/A
7-1: 71031926A/G
7-1: 71066710A/T
7-1: 71164422T/C
7-1: 71186834G/C
7-1: 71229993G/A
7-1: 71248931C/T
7-1: 71280721A/C
7-1: 71340674A/T
7-1: 71396611G/T
7-1: 71425228C/A
7-1: 71470503T/C
7-1: 71506677A/G
7-1: 71535481G/A
7-1: 71650737G/A
7-1: 71747258C/T
7-1: 71803074A/G
7-1: 71882940C/T
7-1: 71985199G/T
7-1: 71987114G/T
7-1: 72108902T/A
7-1: 72261732G/A
7-1: 72269441C/T
7-1: 72319432G/A
7-1: 72427038G/A
7-1: 72458145C/G
7-1: 72537221G/A
7-1: 72599809C/T
7-1: 72678840A/T
7-1: 72775445C/A
7-1: 72887453A/G
7-1: 72911624C/T
7-1: 72989540C/T
7-1: 73021878G/A
7-1: 73048969A/G
7-1: 73092955A/G
7-1: 73131417A/C
7-1: 73194764T/A
7-1: 73215166T/G
7-1: 73245715A/G
7-1: 73292562T/C
7-1: 73448198G/T
7-1: 73455369C/T
7-1: 73523425C/T
7-1: 73615294G/A
7-1: 73694401G/A
7-1: 73767765T/G
7-1: 73818090C/G
7-1: 74079766T/C
7-1: 74102307G/T
7-1: 74114331A/G
7-1: 74155253G/A
7-1: 74190754T/G
7-1: 74249654C/T
7-1: 74314527G/A
7-1: 74336939G/A
7-1: 74374530A/G
7-1: 74404177C/T
7-1: 74451549G/A
7-1: 74456218C/T
7-1: 74461898G/A
7-1: 74475202A/G
7-1: 74486069C/T
7-1: 74518674A/G
7-1: 74564177A/G
7-1: 74589034C/T
7-1: 74638962C/T
7-1: 74655769C/A
7-1: 74664710G/A
7-1: 74667681C/G
7-1: 74709269G/A
7-1: 74719340G/A
7-1: 74724413G/C
7-1: 74736658G/A
7-1: 74744951G/C
7-1: 74761431A/T
7-1: 74832412T/A
7-1: 74923229A/G
7-1: 74923404A/T
7-1: 74923539T/C
7-1: 74923702C/G
7-1: 74923766G/T
7-1: 74924683G/A
7-1: 74952475T/C
7-1: 74982595A/C
7-1: 75019417A/C
7-1: 75061213T/G
7-1: 75098666C/G
7-1: 75118157G/A
7-1: 75144744T/C
7-1: 75171377T/A
7-1: 75225970C/G
7-1: 75300759G/A
7-1: 75353034C/T
7-1: 75512748A/C
7-1: 75576447T/C
7-1: 75592236C/T
7-1: 75753575C/T
7-1: 75805135T/C
7-1: 75831399C/T
7-1: 75867079T/G
7-1: 75974217G/A
7-1: 76119257G/A
7-1: 76165655A/G
7-1: 76197652T/C
7-1: 76263775C/A
7-1: 76264616G/A
7-1: 76264834T/C
7-1: 76265248A/G
7-1: 76265269T/C
7-1: 76265566A/G
7-1: 76265692C/T
7-1: 76265923G/A
7-1: 76266227G/A
7-1: 76266428G/T
7-1: 76266574T/C
7-1: 76266899C/T
7-1: 76267022G/A
7-1: 76267255C/T
7-1: 76267359C/T
7-1: 76634145T/A
7-1: 76720943A/G
7-1: 76725734T/C
7-1: 76786037C/T
7-1: 76960928G/A
7-1: 77093803C/A
7-1: 77103876T/A
7-1: 77232839G/A
7-1: 77270378T/C
7-1: 77308032A/G
7-1: 77335958A/G
7-1: 77484421G/A
7-1: 77542272T/A
7-1: 77585278G/A
7-1: 77629300G/A
7-1: 77697821A/G
7-1: 77742309T/G
7-1: 77882584G/T
7-1: 77913178G/A
7-1: 77977683C/T
7-1: 78029874T/A
7-1: 78109100T/A
7-1: 78255923G/C
7-1: 78428589A/G
7-1: 78566954A/G
7-1: 78568546C/T
7-1: 78782201C/A
7-1: 78787422C/A
7-1: 78824289A/C
7-1: 78875706A/C
7-1: 78916017G/A
7-1: 78927304A/T
7-1: 79066542G/C
7-1: 79120328A/G
7-1: 79279327T/A
7-1: 79340775G/A
7-1: 79409820T/A
7-1: 79538312G/A
7-1: 79624420T/C
7-1: 79691199A/C
7-1: 79787495T/C
7-1: 79852925T/C
7-1: 80038190G/A
7-1: 80133044T/C
7-1: 80229564T/G
7-1: 80452764A/G
7-1: 80530528T/A
7-1: 80624276C/T
7-1: 80732935A/C
7-1: 80827356T/A
7-1: 80874801A/G
7-1: 80903034A/G
7-1: 80995150A/C
7-1: 81048414G/T
7-1: 81102013T/A
7-1: 81113734G/A
7-2: 3788T/G
7-2: 222960T/A
7-2: 585542G/A
7-2: 597595T/A
7-2: 600201C/T
7-2: 672258G/A
7-2: 902659C/T
7-2: 989248A/T
7-2: 1007042C/A
7-2: 1049075C/G
7-2: 1183009T/C
7-2: 1559925T/G
7-2: 1566014T/C
7-2: 1669230C/T
7-2: 1911566G/C
7-2: 1914390C/T
7-2: 1954906T/A
7-2: 1976070T/A
7-2: 1989238G/A
7-2: 2000689C/T
7-2: 2072579A/C
7-2: 2082917G/A
7-2: 2101576G/A
7-2: 2277030C/T
7-2: 2325600G/A
7-2: 2342514G/A
7-2: 2552510C/T
7-2: 2736268T/A
7-2: 2902557A/T
7-2: 2943513G/C
7-2: 3241019C/T
7-2: 3241310C/T
7-2: 3242106T/A
7-2: 3242145T/A
7-2: 3242178C/T
7-2: 3242289C/T
7-2: 3242397G/A
7-2: 3252830G/A
7-2: 3253037C/T
7-2: 3253310G/T
7-2: 3253351T/C
7-2: 3253376G/A
7-2: 3253418G/T
7-2: 3253581G/A
7-2: 3254161A/G
7-2: 3254264G/A
7-2: 3254565G/A
7-2: 3254566C/T
7-2: 3255084A/T
7-2: 3255141T/C
7-2: 3255261T/C
7-2: 3255735A/C
7-2: 3256608G/A
7-2: 3442997A/G
7-2: 3443531C/T
7-2: 3497474G/A
7-2: 3808649C/T
7-2: 3808665C/A
7-2: 3808691T/A
7-2: 3825270T/G
7-2: 3833127T/A
7-2: 3979633G/C
7-2: 3992305A/C
7-2: 4005484T/G
7-2: 4149457T/A
7-2: 4171407G/A
7-2: 4243357G/C
7-2: 4324146T/C
7-2: 4430950A/G
7-2: 4446793T/C
7-2: 4461055C/T
7-2: 4571929G/A
7-2: 4822168C/T
7-2: 5289294A/G
7-2: 5386493G/A
7-2: 5401072G/A
7-2: 5445164A/C
7-2: 5783560T/C
7-2: 5866734T/G
7-2: 6045725A/G
7-2: 6059821C/T
7-2: 6062064T/C
7-2: 6175536C/T
7-2: 6177110A/G
7-2: 6474236A/T
7-2: 6562491A/T
7-2: 6583075C/T
7-2: 6730888C/G
7-2: 6766941T/C
7-2: 6767228G/A
7-2: 6801238T/G
7-2: 6816097T/C
7-2: 6839022C/T
7-2: 6873507A/G
7-2: 6965772A/C
7-2: 7066028A/G
7-2: 7075003G/T
7-2: 7118332G/A
7-2: 7155192C/A
7-2: 7219947A/G
7-2: 7319847G/T
7-2: 7354049C/T
7-2: 7370243G/C
7-2: 7514415G/A
7-2: 7526511T/A
7-2: 7612126A/T
7-2: 7654338T/C
7-2: 7749906C/G
7-2: 7797479A/G
7-2: 7863756A/G
7-2: 7914529C/T
7-2: 7944200A/C
7-2: 7945019C/A
7-2: 7966940A/G
7-2: 7967161A/C
7-2: 7967165G/A
7-2: 7967173A/C
7-2: 7967194C/T
7-2: 7967197C/A
7-2: 8196233T/G
7-2: 8214376T/C
7-2: 8255782T/A
7-2: 8286913T/A
7-2: 8289315T/C
7-2: 8300461G/T
7-2: 8325531A/G
7-2: 8355562A/G
7-2: 8400804G/T
7-2: 8447341C/T
7-2: 8518491T/A
7-2: 8535486T/A
7-2: 8565794C/T
7-2: 8734026A/C
7-2: 8734035A/T
7-2: 8739503G/T
7-2: 8781078T/A
7-2: 8782224G/T
7-2: 8804424A/G
7-2: 8820325C/T
7-2: 8957729A/G
7-2: 8968198A/T
7-2: 8981147A/G
7-2: 9010850G/T
7-2: 9027874A/C
7-2: 9119826A/T
7-2: 9163830C/G
7-2: 9223253G/A
7-2: 9272994G/T
7-2: 9316217C/T
7-2: 9339884A/G
7-2: 9459347C/T
7-2: 9537236A/T
7-2: 9561668C/A
7-2: 9596475C/T
7-2: 9612933A/G
7-2: 9613040T/C
7-2: 9625191T/C
7-2: 9648714T/A
7-2: 9649455A/G
7-2: 9660159C/T
7-2: 9673825T/A
7-2: 9674576T/C
7-2: 9675088T/C
7-2: 9675134T/A
7-2: 9730471A/G
7-2: 9739756C/T
7-2: 9794485G/C
7-2: 9832281C/T
7-2: 9843870T/G
7-2: 9859285G/T
7-2: 9875735C/T
7-2: 9895696G/C
7-2: 9937764A/T
7-2: 9942496C/A
7-2: 9976687G/A
7-2: 9999196T/A
7-2: 10003292A/G
7-2: 10065043C/T
7-2: 10066850A/G
7-2: 10078235C/T
7-2: 10089933A/G
7-2: 10100620A/C
7-2: 10130418G/A
7-2: 10145709T/A
7-2: 10172389A/C
7-2: 10218222A/T
7-2: 10264986C/T
7-2: 10337695C/T
7-2: 10371721A/T
7-2: 10426446G/A
7-2: 10482817C/T
7-2: 10531409G/T
7-2: 10559572C/T
7-2: 10883609C/T
7-2: 10906935T/C
7-2: 10950223G/A
7-2: 10974440A/G
7-2: 11112214T/A
7-2: 11163078C/A
7-2: 11225668A/G
7-2: 11363236C/T
7-2: 11395057C/A
7-2: 11427231C/T
7-2: 11428271T/C
7-2: 11450760C/G
7-2: 11496824T/C
7-2: 11547917G/T
7-2: 11551513T/C
7-2: 11559155T/G
7-2: 11564403G/A
7-2: 11564588T/C
7-2: 11564692C/T
7-2: 11566971A/G
7-2: 11567150G/A
7-2: 11567900C/A
7-2: 11568794G/A
7-2: 11572783T/C
7-2: 11610488A/T
7-2: 11624357T/G
7-2: 11649420T/G
7-2: 11680031A/G
7-2: 11723325C/T
7-2: 11752961C/T
7-2: 11795154T/C
7-2: 11821050A/G
7-2: 11841018C/T
7-2: 11898242T/C
7-2: 11950521A/C
7-2: 11951447C/T
7-2: 11961613G/A
7-2: 11989880T/C
7-2: 12000525A/C
7-2: 12052104C/T
7-2: 12052249A/T
7-2: 12123522C/T
7-2: 12207417T/A
7-2: 12256634G/A
7-2: 12332541C/T
7-2: 12382221G/C
7-2: 12475747C/T
7-2: 12541091T/A
7-2: 12581056G/A
7-2: 12592374C/G
7-2: 12649833A/G
7-2: 12684338G/T
7-2: 12746525C/A
7-2: 12747226C/T
7-2: 12747241G/C
7-2: 12747847G/A
7-2: 12747851G/A
7-2: 12747952T/C
7-2: 12748090G/T
7-2: 12774961T/C
7-2: 12844499G/A
7-2: 12854541T/C
7-2: 12867596T/C
7-2: 12949382G/A
7-2: 12955858C/T
7-2: 12964489G/T
7-2: 13202106G/T
7-2: 13203480A/C
7-2: 13376070A/G
7-2: 13434693T/C
7-2: 13476805A/T
7-2: 13531088C/A
7-2: 13596315T/A
7-2: 13665838C/A
7-2: 13702756T/C
7-2: 13728795C/A
7-2: 13850075G/A
7-2: 13866568T/C
7-2: 13921439G/A
7-2: 13968462G/T
7-2: 13983794G/C
7-2: 14043391G/A
7-2: 14137026G/T
7-2: 14148805C/A
7-2: 14232014A/T
7-2: 14302335T/C
7-2: 14353253T/G
7-2: 14395096T/A
7-2: 14407650C/A
7-2: 14432682C/A
7-2: 14463507T/G
7-2: 14541119A/C
7-2: 14613107C/T
7-2: 14655230A/G
7-2: 14718432A/G
7-2: 14754245T/A
7-2: 14793477C/T
7-2: 14829124C/T
7-2: 14936759C/A
7-2: 14947940T/C
7-2: 14962115A/G
7-2: 15020931C/T
7-2: 15211623T/A
7-2: 15254052A/G
7-2: 15491622G/A
7-2: 15673964G/C
7-2: 15902209G/A
7-2: 16002437C/T
7-2: 16004374G/A
7-2: 16034216T/C
7-2: 16175034C/A
7-2: 16289834C/T
7-2: 16308039C/T
7-2: 16337852A/G
7-2: 16420313A/G
7-2: 16425507A/T
7-2: 16474164G/C
7-2: 16520992G/T
7-2: 16521497A/G
7-2: 16521584A/G
7-2: 16557760G/A
7-2: 16578230A/T
7-2: 16602577A/G
7-2: 16730679T/G
7-2: 16810548C/T
7-2: 16926768C/T
7-2: 16978374A/G
7-2: 16979921G/A
7-2: 17021969C/A
7-2: 17123691A/G
7-2: 17130761A/C
7-2: 17130818A/G
7-2: 17132520C/T
7-2: 17132907A/G
7-2: 17134515C/T
7-2: 17174236T/A
7-2: 17367725G/A
7-2: 17394392G/A
7-2: 17491745A/C
7-2: 17552191G/A
7-2: 17789986A/G
7-2: 17845636C/T
7-2: 17868077A/G
7-2: 17951855A/G
7-2: 17987292T/G
7-2: 17995947A/G
7-2: 18012431T/C
7-2: 18014246T/C
7-2: 18014779C/A
7-2: 18059630G/T
7-2: 18092463G/A
7-2: 18162110T/A
7-2: 18198837T/A
7-2: 18209047C/T
7-2: 18231385C/T
7-2: 18274509C/T
7-2: 18331553A/T
7-2: 18395725A/G
7-2: 18554676A/G
7-2: 18575579G/A
7-2: 18595593C/G
7-2: 18626521T/G
7-2: 18645837G/T
7-2: 18662154G/A
7-2: 18664727C/T
7-2: 18665662G/A
7-2: 18710798C/T
7-2: 18808840T/C
7-2: 18823245C/A
7-2: 18874161T/G
7-2: 18901942C/T
7-2: 18914748G/A
7-2: 18931218G/A
7-2: 18945524C/T
7-2: 18984942C/T
7-2: 19015308G/A
7-2: 19043591A/C
7-2: 19106902G/A
7-2: 19107223G/A
7-2: 19107420C/T
7-2: 19110321A/G
7-2: 19110771C/T
7-2: 19110951T/C
7-2: 19111301C/G
7-2: 19112079T/G
7-2: 19116731G/A
7-2: 19122861G/T
7-2: 19195377C/T
7-2: 19284555A/G
7-2: 19317345A/G
7-2: 19330380T/C
7-2: 19362023T/C
7-2: 19374619C/T
7-2: 19391144C/T
7-2: 19454994T/G
7-2: 19480809T/A
7-2: 19517706T/C
7-2: 19552601C/A
7-2: 19628484G/T
7-2: 19671203G/T
7-2: 19698483T/A
7-2: 19727147T/C
7-2: 19741516T/A
7-2: 19753078T/C
7-2: 19767920T/C
7-2: 19775017G/C
7-2: 19775150T/C
7-2: 19775276G/A
7-2: 19775368A/C
7-2: 19793026C/T
7-2: 19823199G/A
7-2: 19940511A/C
7-2: 19948524T/A
7-2: 19967430T/C
7-2: 20021036C/T
7-2: 20021045C/T
7-2: 20145011C/T
7-2: 20223820T/A
7-2: 20287300T/C
7-2: 20325978C/A
7-2: 20353874A/G
7-2: 20384817G/A
7-2: 20412480C/T
7-2: 20464706C/T
7-2: 20519006A/C
7-2: 20564441G/A
7-2: 20605348G/A
7-2: 20620826G/A
7-2: 20631179A/G
7-2: 20665262C/A
7-2: 20808148A/T
7-2: 20891479T/C
7-2: 20916039A/T
7-2: 20945886C/T
7-2: 21015219T/G
7-2: 21075323T/C
7-2: 21193146T/G
7-2: 21213945A/T
7-2: 21262582G/T
7-2: 21315973C/T
7-2: 21371062C/G
7-2: 21388360C/G
7-2: 21388986A/T
7-2: 21389071G/T
7-2: 21389306G/A
7-2: 21402513C/T
7-2: 21417117T/C
7-2: 21443675C/T
7-2: 21471152C/G
7-2: 21573257T/G
7-2: 21783135T/C
7-2: 21871023C/T
7-2: 21902073A/G
7-2: 21993358G/C
7-2: 22127715A/G
7-2: 22128953A/G
7-2: 22214899A/T
7-2: 22247760C/T
7-2: 22254261A/C
7-2: 22254373A/G
7-2: 22256362T/A
7-2: 22256456G/T
7-2: 22256512C/T
7-2: 22258079G/A
7-2: 22258124T/A
7-2: 22258478A/C
7-2: 22259129A/T
7-2: 22266207G/T
7-2: 22267157C/T
7-2: 22267170A/G
7-2: 22398255T/G
7-2: 22434475A/C
7-2: 22498908T/G
7-2: 22528081T/G
7-2: 22593885T/A
7-2: 22636170G/A
7-2: 22665596T/A
7-2: 22667545T/A
7-2: 22700538C/T
7-2: 22837190T/A
7-2: 22923653A/G
7-2: 22959040A/G
7-2: 23022436C/T
7-2: 23079917C/T
7-2: 23118813T/C
7-2: 23121511G/T
7-2: 23121696C/A
7-2: 23121702T/C
7-2: 23121717T/C
7-2: 23150175T/C
7-2: 23248085C/G
7-2: 23257612A/C
7-2: 23263966T/A
7-2: 23278324C/A
7-2: 23315434T/A
7-2: 23372707T/C
7-2: 23383671A/C
7-2: 23421994A/G
7-2: 23460097C/T
7-2: 23473192T/G
7-2: 23501347A/T
7-2: 23552475C/T
7-2: 23580796G/T
7-2: 23636162T/A
7-2: 23659259A/T
7-2: 23692873C/T
7-2: 23725056G/A
7-2: 23736013A/T
7-2: 23768908G/A
7-2: 23795912A/C
7-2: 23812678G/A
7-2: 23828030T/C
7-2: 23841560T/A
7-2: 23867361T/C
7-2: 23879884G/A
7-2: 23949910C/T
7-2: 24108939A/T
7-2: 24204179T/A
7-2: 24308327T/C
7-2: 24313062C/T
7-2: 24335844T/A
7-2: 24444566G/A
7-2: 24455086T/C
7-2: 24469949A/G
7-2: 24521369C/T
7-2: 24532120G/T
7-2: 24535276G/A
7-2: 24542173A/T
7-2: 24549662C/A
7-2: 24550253C/T
7-2: 24563404T/A
7-2: 24569860C/T
7-2: 24588096T/C
7-2: 24638349A/T
7-2: 24690747G/A
7-2: 24711610T/C
7-2: 24726053G/T
7-2: 24741520G/T
7-2: 24789082G/T
7-2: 24872066C/T
7-2: 24889115C/A
7-2: 24924026A/T
7-2: 24972877A/C
7-2: 24993452G/A
7-2: 25007419T/A
7-2: 25040709G/A
7-2: 25112889C/T
7-2: 25130969C/T
7-2: 25132298T/C
7-2: 25170485A/G
7-2: 25210658T/A
7-2: 25276469T/C
7-2: 25359615C/T
7-2: 25462734G/A
7-2: 25557408A/C
7-2: 25653529C/T
7-2: 25716307C/A
7-2: 25743175A/G
7-2: 25744471G/A
7-2: 25759150T/C
7-2: 25761374G/A
7-2: 25762464G/A
7-2: 25765861A/C
7-2: 25771399A/T
7-2: 25787995T/C
7-2: 25805987C/G
7-2: 25854005T/C
7-2: 25854081T/C
7-2: 25870031T/C
7-2: 25896558A/T
7-2: 25908945G/A
7-2: 25913010T/C
7-2: 25956598A/G
7-2: 25966843T/C
7-2: 25977639G/C
7-2: 26046391T/A
7-2: 26048977A/T
7-2: 26110593T/A
7-2: 26130691A/T
7-2: 26146580T/C
77-2: 26158211C/T
7-2: 26188719T/C
7-2: 26207593G/A
7-2: 26208192T/A
7-2: 26225711G/C
7-2: 26231290A/T
7-2: 26280146T/A
7-2: 26337183A/G
7-2: 26338044T/C
7-2: 26355446G/T
7-2: 26383037C/G
7-2: 26404990T/C
7-2: 26423313A/T
7-2: 26435876C/T
7-2: 26559602G/T
7-2: 26583319G/A
7-2: 26593805C/A
7-2: 26618925T/C
7-2: 26671320C/A
7-2: 26712143T/A
7-2: 26714537G/A
7-2: 26715401C/A
7-2: 26715406A/T
7-2: 26715511A/G
7-2: 26715657C/T
7-2: 26716401G/T
7-2: 26739923T/C
7-2: 26751126A/G
7-2: 26837376T/G
7-2: 26965765T/A
7-2: 27000124T/G
7-2: 27032356G/T
7-2: 27035998C/T
7-2: 27037238T/A
7-2: 27037265G/A
7-2: 27037664A/C
7-2: 27045973A/G
7-2: 27058451G/T
7-2: 27076882G/A
7-2: 27081179C/A
7-2: 27081570C/T
7-2: 27081692C/T
7-2: 27082068T/C
7-2: 27082088A/G
7-2: 27082466A/T
7-2: 27083295C/A
7-2: 27084826T/C
7-2: 27086206C/T
7-2: 27086751G/A
7-2: 27095032T/C
7-2: 27108630A/G
7-2: 27125903A/G
7-2: 27143612T/C
7-2: 27159892C/T
7-2: 27162904G/A
7-2: 27168609T/C
7-2: 27215965G/A
7-2: 27252942T/A
7-2: 27432416C/T
7-2: 27549363T/A
7-2: 27662810C/T
7-2: 27690873A/C
7-2: 27740267C/T
7-2: 27754817A/C
7-2: 27759400T/A
7-2: 27772753A/G
7-2: 27889439A/T
7-2: 27910525C/A
7-2: 27910590C/T
7-2: 27912902A/G
7-2: 27914079T/C
7-2: 27914081G/A
7-2: 27914115A/G
7-2: 27919007A/C
7-2: 27921672G/T
7-2: 27931859T/C
7-2: 27936222C/A
7-2: 27947604A/G
7-2: 27965112C/G
7-2: 27983296G/T
7-2: 28032146T/C
7-2: 28081105T/C
7-2: 28090000G/T
7-2: 28112601A/T
7-2: 28137589A/T
7-2: 28148326C/T
7-2: 28148555G/A
7-2: 28152247C/G
7-2: 28152342T/G
7-2: 28152347C/T
7-2: 28152354T/C
7-2: 28172469T/C
7-2: 28245762C/T
7-2: 28251789G/A
7-2: 28272067T/A
7-2: 28325477T/A
7-2: 28336468A/T
7-2: 28379966C/T
7-2: 28394898G/A
7-2: 28413526C/T
7-2: 28476339T/G
7-2: 28476536A/C
7-2: 28477234G/T
7-2: 28477369A/G
7-2: 28495816C/T
7-2: 28502318T/C
7-2: 28503743T/C
7-2: 28533919G/A
7-2: 28570070G/A
7-2: 28647903C/T
7-2: 28659920T/C
7-2: 28661222T/C
7-2: 28664815C/T
7-2: 28665482G/A
7-2: 28665644T/G
7-2: 28665669T/C
7-2: 28666087G/A
7-2: 28666150G/C
7-2: 28675359A/G
7-2: 28685674A/T
7-2: 28704194C/G
7-2: 28708046A/G
7-2: 28708373C/A
7-2: 28714368C/A
7-2: 28729128T/C
7-2: 28752125T/C
7-2: 28752188G/A
7-2: 28796332A/T
7-2: 28826538A/G
7-2: 28839051T/C
7-2: 28839261A/G
7-2: 28839376A/G
7-2: 28839480C/T
7-2: 28851686A/G
7-2: 28862772A/T
7-2: 28872049C/T
7-2: 28872837G/A
7-2: 28873515A/G
7-2: 28875343A/C
7-2: 28939662G/C
7-2: 28950209G/A
7-2: 28976172C/T
7-2: 28993611T/G
7-2: 29004986C/T
7-2: 29015431C/T
7-2: 29026438G/A
7-2: 29050760G/A
7-2: 29063818T/A
7-2: 29087580A/G
7-2: 29111249T/A
7-2: 29114877G/A
7-2: 29115389T/C
7-2: 29165269C/A
7-2: 29244697C/G
7-2: 29294323G/A
7-2: 29319153C/A
7-2: 29326889T/C
7-2: 29332076C/T
7-2: 29332772A/T
7-2: 29332786A/C
7-2: 29332900T/A
7-2: 29334416A/G
7-2: 29334551C/T
7-2: 29342599G/A
7-2: 29352542A/G
7-2: 29354609C/G
7-2: 29432945A/G
7-2: 29572245G/T
7-2: 29587857T/G
7-2: 29590528G/A
7-2: 29595130A/C
7-2: 29598866C/G
7-2: 29608231G/A
7-2: 29612333G/C
7-2: 29653249A/C
7-2: 29669895T/C
7-2: 29712246A/G
7-2: 29714894A/C
7-2: 29764899A/G
7-2: 29769255A/C
7-2: 29769386G/A
7-2: 29769566C/T
7-2: 29784343A/T
7-2: 29793260C/A
7-2: 29796684A/G
7-2: 29831302A/G
7-2: 29842516C/G
7-2: 29868357C/T
7-2: 29880310G/A
7-2: 29894364A/C
7-2: 29911777C/G
7-2: 29922883G/A
7-2: 29923095A/G
7-2: 29964968C/A
7-2: 30053806T/C
7-2: 30064969G/A
7-2: 30142043A/C
7-2: 30158759C/T
7-2: 30178591A/C
7-2: 30200304G/A
7-2: 30203930C/G
7-2: 30225310G/C
7-2: 30312959C/T
7-2: 30313847G/A
7-2: 30324494T/C
7-2: 30326607A/G
7-2: 30326709G/A
7-2: 30347192C/T
7-2: 30375407G/C
7-2: 30385459C/A
7-2: 30417804T/G
7-2: 30526632G/A
7-2: 30548987A/G
7-2: 30625006G/A
7-2: 30628326T/C
7-2: 30652148T/A
7-2: 30678644C/A
7-2: 30722223G/A
7-2: 30750374T/A
7-2: 30772582C/T
7-2: 30775516C/T
7-2: 30783436G/A
7-2: 30804234C/T
7-2: 30834965G/A
7-2: 30848798T/C
7-2: 30864289C/T
7-2: 30879224G/A
7-2: 30881081A/C
7-2: 30906676A/G
7-2: 30940611C/T
7-2: 30976595T/A
7-2: 31027875T/C
7-2: 31092593A/T
7-2: 31157870G/C
7-2: 31187550G/A
7-2: 31260602C/G
7-2: 31293865C/T
7-2: 31317420G/A
7-2: 31398621C/A
7-2: 31446065C/A
7-2: 31725617T/G
7-2: 31757762T/G
7-2: 31955544C/T
7-2: 32022947A/G
7-2: 32044276A/C
7-2: 32202250A/G
7-2: 32213037C/T
7-2: 32237622C/T
7-2: 32285826A/T
7-2: 32306394T/C
7-2: 32428516A/G
7-2: 32614850G/A
7-2: 32640852T/A
7-2: 32750271G/A
7-2: 32861169A/T
7-2: 32970949T/A
7-2: 33121848A/T
7-2: 33129886G/A
7-2: 33150148C/G
7-2: 33169061T/C
7-2: 33195766C/G
7-2: 33200745C/T
7-2: 33201444T/C
7-2: 33201573C/A
7-2: 33203076G/A
7-2: 33205092A/G
7-2: 33205350A/T
7-2: 33231938G/C
7-2: 33234686C/T
7-2: 33234717A/G
7-2: 33299680C/T
7-2: 33314066A/T
7-2: 33317022G/A
7-2: 33360466A/G
7-2: 33453583A/G
7-2: 33486457G/A
7-2: 33501007G/A
7-2: 33562929T/C
7-2: 33607777C/T
7-2: 33639639T/A
7-2: 33657355T/A
7-2: 33731152G/A
7-2: 33838507T/G
7-2: 33839014G/A
7-2: 33839032T/A
7-2: 33839036C/T
7-2: 33839047G/T
7-2: 33929804C/T
7-2: 33947899C/T
7-2: 33971584C/T
7-2: 34001969C/T
7-2: 34033388T/C
7-2: 34033440G/C
7-2: 34035802C/T
7-2: 34036220T/A
7-2: 34037140T/C
7-2: 34038288T/C
7-2: 34038410A/G
7-2: 34038510T/C
7-2: 34038795A/T
7-2: 34039410C/T
7-2: 34043119A/G
7-2: 34043404A/G
7-2: 34043794A/T
7-2: 34044084A/T
7-2: 34044132G/C
7-2: 34066695A/C
7-2: 34090033C/T
7-2: 34154168T/C
7-2: 34202695A/T
7-2: 34236581A/G
7-2: 34267557C/T
7-2: 34279865G/A
7-2: 34295516T/A
7-2: 34309734T/C
7-2: 34519571T/A
7-2: 34529484T/C
7-2: 34556120A/C
7-2: 34568325T/C
7-2: 34584413C/T
7-2: 34693941G/A
7-2: 34717686C/A
7-2: 34780871G/A
7-2: 34820254G/A
7-2: 34866003C/T
7-2: 34890966C/T
7-2: 34932773C/G
7-2: 35121857C/T
7-2: 35137860G/A
7-2: 35226084C/T
7-2: 35354474A/T
7-2: 35404200A/T
7-2: 35460525G/A
7-2: 35460608G/C
7-2: 35470625T/G
7-2: 35494230C/T
7-2: 35534852A/C
7-2: 35583559A/T
7-2: 35620961C/T
7-2: 35637073C/G
7-2: 35649715G/A
7-2: 35670405G/T
7-2: 35671175G/A
7-2: 35679963T/C
7-2: 35692293T/C
7-2: 35730807G/A
7-2: 35738994A/G
7-2: 35740869A/C
7-2: 35759498G/A
7-2: 35771114G/T
7-2: 35773775C/T
7-2: 35792460A/G
7-2: 35810837C/G
7-2: 35826462G/A
7-2: 35857967A/G
7-2: 35887755G/A
7-2: 36021936T/A
7-2: 36245293A/C
7-2: 36310448G/C
7-2: 36364334T/A
7-2: 36364746C/T
7-2: 36364834C/T
7-2: 36365105T/C
7-2: 36365341G/A
7-2: 36365452G/A
7-2: 36436202A/C
7-2: 36453479C/A
7-2: 36560022G/A
7-2: 36744293G/A
7-2: 36856503A/G
7-2: 36947117G/T
7-2: 37031766C/A
7-2: 37048992G/T
7-2: 37158592A/T
7-2: 37160934A/G
7-2: 37182943T/C
7-2: 37183174A/T
7-2: 37183184A/G
7-2: 37183210C/T
7-2: 37238984T/A
7-2: 37488775G/A
7-2: 37716165G/A
7-2: 37920335C/T
7-2: 37947178C/A
7-2: 38247779G/A
7-2: 38275204C/T
7-2: 38315872G/A
7-2: 38327695C/T
7-2: 38355660A/G
7-2: 38403143G/C
7-2: 38435731G/A
7-2: 38437262G/A
7-2: 38447796G/A
7-2: 38448633G/T
7-2: 38463568T/A
7-2: 38494650A/C
7-2: 38544287A/G
7-2: 38591349G/A
7-2: 38615034T/C
7-2: 38725022A/C
7-2: 38726979A/C
7-2: 38751044G/A
7-2: 38770571C/G
7-2: 38795212G/A
7-2: 39098476G/A
7-2: 39099742T/C
7-2: 39191988C/T
7-2: 39218089C/T
7-2: 39246205C/T
7-2: 39260789C/A
7-2: 39276321A/T
7-2: 39320852A/C
7-2: 39350166A/C
7-2: 39354657A/G
7-2: 39368782C/T
7-2: 39373371T/A
7-2: 39404996G/A
7-2: 39422201A/C
7-2: 39457586G/A
7-2: 39488371G/A
7-2: 39522489T/C
7-2: 39558339C/T
7-2: 39566766C/A
7-2: 39569731G/A
7-2: 39570995G/A
7-2: 39626605A/T
7-2: 39653232C/T
7-2: 39669196A/T
7-2: 39673554T/C
7-2: 39707410T/C
7-2: 39722787T/A
7-2: 39724815T/C
7-2: 39764314C/A
7-2: 39777726G/A
7-2: 39890500A/G
7-2: 39932093A/G
7-2: 39963737G/C
7-2: 40025140G/T
7-2: 40364151T/G
7-2: 40379726A/T
7-2: 40416151C/A
7-2: 40445286T/A
7-2: 40446329T/C
7-2: 40475020T/C
7-2: 40507562T/A
7-2: 40534555C/A
7-2: 40586184A/T
7-2: 40662412T/A
7-2: 40678562C/T
7-2: 40813165T/G
7-2: 40823877C/T
7-2: 40871869A/T
7-2: 40901101A/T
7-2: 40914501G/A
7-2: 40984819C/T
7-2: 41010193G/A
7-2: 41054159A/G
7-2: 41062848G/A
7-2: 41063022G/A
7-2: 41063188G/A
7-2: 41063237C/T
7-2: 41069027T/C
7-2: 41092108C/T
7-2: 41147986A/T
7-2: 41178159T/A
7-2: 41192289A/T
7-2: 41218164C/T
7-2: 41240826G/A
7-2: 41252990T/C
7-2: 41254188A/T
7-2: 41273361T/A
7-2: 41298734G/A
7-2: 41331818A/T
7-2: 41399811C/G
7-2: 41425895A/G
7-2: 41483692C/T
7-2: 41517069G/C
7-2: 41543086C/G
7-2: 41584761G/A
7-2: 41648767G/A
7-2: 41664360A/G
7-2: 41692319A/T
7-2: 41726998G/A
7-2: 41741697A/C
7-2: 41743126T/A
7-2: 41759763T/A
7-2: 41787216G/A
7-2: 41805603A/G
7-2: 41811790T/A
7-2: 41812467T/C
7-2: 41812850C/T
7-2: 41812894T/C
7-2: 41813921A/T
7-2: 41814673T/G
7-2: 41816340C/G
7-2: 41827497G/A
7-2: 41831098A/C
7-2: 41839551A/G
7-2: 41873601A/T
7-2: 41883731G/A
7-2: 41901328C/T
7-2: 41923285A/G
7-2: 41924066C/A
7-2: 41924229C/G
7-2: 41932306A/G
7-2: 41933660C/A
7-2: 41933703C/A
7-2: 41933858C/G
7-2: 41935332G/A
7-2: 41977585A/T
7-2: 42029660A/G
7-2: 42066938A/G
7-2: 42092050C/T
7-2: 42162207C/A
7-2: 42178117T/A
7-2: 42229255G/A
7-2: 42284017C/G
7-2: 42364487T/C
7-2: 42475818C/T
7-2: 42550693G/A
7-2: 42667620A/C
7-2: 42681654G/A
7-2: 42695520A/G
7-2: 42860590T/A
7-2: 42924381A/C
7-2: 42988726A/G
7-2: 43144998G/A
7-2: 43156932G/A
7-2: 43213238A/G
7-2: 43379501G/T
7-2: 43398126G/A
7-2: 43421082G/T
7-2: 43467311C/T
7-2: 43503843G/C
7-2: 43571742C/A
7-2: 43725014T/C
7-2: 43727858T/A
7-2: 43827111T/G
7-2: 44033141C/T
7-2: 44123756G/C
7-2: 44126082C/T
7-2: 44127000A/G
7-2: 44183716A/T
7-2: 44329735C/A
7-2: 44345817C/T
7-2: 44357666T/A
7-2: 44409160G/A
7-2: 44456983C/A
7-2: 44618334G/A
7-2: 44631074G/A
7-2: 44656791G/A
7-2: 44769012G/A
7-2: 44838327A/G
7-2: 44878878G/A
7-2: 44953642C/T
7-2: 44960033C/T
7-2: 44995722G/C
7-2: 45059241T/G
7-2: 45157564C/G
7-2: 45181185G/C
7-2: 45220233G/T
7-2: 45235827T/A
7-2: 45256047G/A
7-2: 45277870C/T
7-2: 45312869G/A
7-2: 45358604G/T
7-2: 45388222C/T
7-2: 45435952G/T
7-2: 45455673G/A
7-2: 45537088T/C
7-2: 45559105A/G
7-2: 45597593C/T
7-2: 45646544C/A
7-2: 45647239A/T
7-2: 45658370A/G
7-2: 45658427G/A
7-2: 45663431G/A
7-2: 45683629G/C
7-2: 45714256G/A
7-2: 45746650A/G
7-2: 45787260G/A
7-2: 45798100G/A
7-2: 45798286A/C
7-2: 45800156A/T
7-2: 45800289G/T
7-2: 45854924C/T
7-2: 45857595T/A
7-2: 45870666C/T
7-2: 45875207T/A
7-2: 45902048C/A
7-2: 45904658C/T
7-2: 45990299T/C
7-2: 46030025G/A
7-2: 46213319A/G
7-2: 46354433T/A
7-2: 46365019T/C
7-2: 46370639C/T
7-2: 46405829A/G
7-2: 46453758A/G
7-2: 46463845T/A
7-2: 46559127C/G
7-2: 46702393T/G
7-2: 46722784G/A
7-2: 46778174T/A
7-2: 46837452C/T
7-2: 47102224T/A
7-2: 47154415G/A
7-2: 47188688A/G
7-2: 47260657G/T
7-2: 47451056T/C
7-2: 47451951A/T
7-2: 47463944G/A
7-2: 47511745G/T
7-2: 47539909A/G
7-2: 47570108A/C
7-2: 47582927A/C
7-2: 47624399C/T
7-2: 47650583G/A
7-2: 47679810A/C
7-2: 47777302G/T
7-2: 47833506G/A
7-2: 47931001A/G
7-2: 47966168T/G
7-2: 48022216T/G
7-2: 48024756A/G
7-2: 48040177C/A
7-2: 48117345C/A
7-2: 48198802C/T
7-2: 48260217A/G
7-2: 48415083A/T
7-2: 48493447A/T
7-2: 48576521G/A
7-2: 48611872T/C
7-2: 48701294C/T
7-2: 48761732G/A
7-2: 48793931A/G
7-2: 48847020T/C
7-2: 48972981A/T
7-2: 49049901T/A
7-2: 49120036G/T
7-2: 49171251T/C
7-2: 49182985T/G
7-2: 49214616G/T
7-2: 49215504G/A
7-2: 49267922G/T
7-2: 49404882T/C
7-2: 49438009G/A
7-2: 49472952T/G
7-2: 49473258C/T
7-2: 49554826T/C
7-2: 49600657T/G
7-2: 49600897C/T
7-2: 49600924T/.
7-2: 49657109G/A
7-2: 49664340A/G
7-2: 49670213T/A
7-2: 49683326C/T
7-2: 49683949C/T
7-2: 49684707G/A
7-2: 49694845T/C
7-2: 49706086C/T
7-2: 49718839C/A
7-2: 49764746G/C
7-2: 49775026T/C
7-2: 49786384A/T
7-2: 49911712G/A
7-2: 49994894G/T
7-2: 50056532C/T
7-2: 50066552C/T
7-2: 50099020C/T
7-2: 50111020C/T
7-2: 50116599G/A
7-2: 50135041A/T
7-2: 50154485G/A
7-2: 50208526C/T
7-2: 50238433A/T
7-2: 50303428T/C
7-2: 50349044A/C
7-2: 50350045G/A
7-2: 50389967C/T
7-2: 50407172C/T
7-2: 50433705C/A
7-2: 50434955A/G
7-2: 50434994C/G
7-2: 50461068C/T
7-2: 50529686A/G
7-2: 50568299A/G
7-2: 50596639C/T
7-2: 50643327A/T
7-2: 50669748G/T
7-2: 50718625G/A
7-2: 50781907C/T
7-2: 50806990C/T
7-2: 50834510T/C
7-2: 50862539A/C
7-2: 50891335A/G
7-2: 50960019A/T
7-2: 51000337T/C
7-2: 51033890C/T
7-2: 51050391A/C
7-2: 51060984T/G
7-2: 51135912T/C
7-2: 51144216C/T
7-2: 51214746C/A
7-2: 51263400A/G
7-2: 51317332T/G
7-2: 51342506C/T
7-2: 51427546G/T
7-2: 51467593G/T
7-2: 51467602A/T
7-2: 51467739G/A
7-2: 51467848T/C
7-2: 51467950G/T
7-2: 51481090T/C
7-2: 51492968A/G
7-2: 51498268C/G
7-2: 51498436A/G
7-2: 51498443T/C
7-2: 51499011C/T
7-2: 51499043T/G
7-2: 51513084G/A
7-2: 51527892C/T
7-2: 51557864G/T
7-2: 51588088T/C
7-2: 51630789T/C
7-2: 51732492G/A
7-2: 51855023G/A
7-2: 51856283G/C
7-2: 51869962G/T
7-2: 51976051T/G
7-2: 52009819C/T
7-2: 52035543C/T
7-2: 52060546G/A
7-2: 52073286T/A
7-2: 52084326C/G
7-2: 52110915C/T
7-2: 52141229C/T
7-2: 52158987A/C
7-2: 52159355A/T
7-2: 52162588T/C
7-2: 52182431A/C
7-2: 52195522T/A
7-2: 52203923A/C
7-2: 52206129C/G
7-2: 52246237C/T
7-2: 52258462T/C
7-2: 52272390G/A
7-2: 52349512A/C
7-2: 52419009A/G
7-2: 52454256A/G
7-2: 52457338A/T
7-2: 52457339G/C
7-2: 52468658C/T
7-2: 52542641G/A
7-2: 52621442G/T
7-2: 52696025A/G
7-2: 52773206C/T
7-2: 52780359G/A
7-2: 52783990A/T
7-2: 52975996C/A
7-2: 53001621G/A
7-2: 53035526C/T
7-2: 53070561C/T
7-2: 53096906A/G
7-2: 53249374C/A
7-2: 53418956T/C
7-2: 53474999C/G
7-2: 53475607T/A
7-2: 53475931C/G
7-2: 53514914T/G
7-2: 53515069T/A
7-2: 53515514C/A
7-2: 53515539T/A
7-2: 53557976G/A
7-2: 53561419T/A
7-2: 53596644G/A
7-2: 53609387A/G
7-2: 53609733A/T
7-2: 53609801A/C
7-2: 53622824G/T
7-2: 53623059T/G
7-2: 53721374A/G
7-2: 53723142T/C
7-2: 53723570A/T
7-2: 53723700T/C
7-2: 53727272G/T
7-2: 53742809G/A
7-2: 53743604G/C
7-2: 53750872A/T
7-2: 53751023A/C
7-2: 53751160T/C
7-2: 53751312G/T
7-2: 53751452G/C
7-2: 53751553C/T
7-2: 53751656G/A
7-2: 53751759G/A
7-2: 53751863A/G
7-2: 53751907A/G
7-2: 53752279G/A
7-2: 53752457A/T
7-2: 53752467A/G
7-2: 53753323G/A
7-2: 53753446G/T
7-2: 53753620A/G
7-2: 53755151A/T
7-2: 53761211T/C
7-2: 53761716C/T
7-2: 53785462T/A
7-2: 53820097T/A
7-2: 53844423T/C
7-2: 53844933A/G
7-2: 53845200A/G
7-2: 53853183A/T
7-2: 53853296G/A
7-2: 53853526T/G
7-2: 53861265A/G
7-2: 53920215G/A
7-2: 53920810T/C
7-2: 53925107A/T
7-2: 53925506T/A
7-2: 53925591A/G
7-2: 53925673T/C
7-2: 53940006C/T
7-2: 53943698T/G
7-2: 53944132C/T
7-2: 53944979T/C
7-2: 53947893T/C
7-2: 53947943T/C
7-2: 53948462A/T
7-2: 53948572A/T
7-2: 53949062T/C
7-2: 53949445T/A
7-2: 53949489C/T
7-2: 53949958C/T
7-2: 53952054C/G
7-2: 53952292T/A
7-2: 53952557C/A
7-2: 53954258G/A
7-2: 54002757A/G
7-2: 54049487G/T
7-2: 54116916T/G
7-2: 54176437A/C
7-2: 54195547C/A
7-2: 54196268T/C
7-2: 54199101A/G
7-2: 54212253G/T
7-2: 54216678A/T
7-2: 54217002C/T
7-2: 54218696A/C
7-2: 54229378G/C
7-2: 54242249C/T
7-2: 54268926C/G
7-2: 54274473A/C
7-2: 54285043C/T
7-2: 54302409C/T
7-2: 54307609A/G
7-2: 54307619T/A
7-2: 54347446C/T
7-2: 54369281G/T
7-2: 54491831T/C
7-2: 54515702C/T
7-2: 54575090T/C
7-2: 54585472T/A
7-2: 54595856G/A
7-2: 54605004C/T
7-2: 54605041T/C
7-2: 54605105C/A
7-2: 54605833G/A
7-2: 54607719A/G
7-2: 54617441A/C
7-2: 54617544G/A
7-2: 54617788C/T
7-2: 54617918T/A
7-2: 54618308G/A
7-2: 54618427T/A
7-2: 54618579C/T
7-2: 54618684G/T
7-2: 54618955A/G
7-2: 54619019T/C
7-2: 54637433T/A
7-2: 54685926G/A
7-2: 54728646G/A
7-2: 54763635C/T
7-2: 54816160G/A
7-2: 54870376A/G
7-2: 54934840C/T
7-2: 54956423C/A
7-2: 54967936A/G
7-2: 54989060G/A
7-2: 55000173A/T
7-2: 55129521G/C
7-2: 55163384A/G
7-2: 55173844A/T
7-2: 55195309A/G
7-2: 55306827G/A
7-2: 55344640G/A
7-2: 55376531A/G
7-2: 55376638A/G
7-2: 55376680T/G
7-2: 55377789G/A
7-2: 55379693G/A
7-2: 55379843G/T
7-2: 55379990T/C
7-2: 55381075G/A
7-2: 55403137G/T
7-2: 55434724A/G
7-2: 55468178C/T
7-2: 55483816C/T
7-2: 55532578A/G
7-2: 55677864C/A
7-2: 55711026T/A
7-2: 55749673A/G
7-2: 55793979A/G
7-2: 55813838A/C
7-2: 55829967A/G
7-2: 55831782C/T
7-2: 55921357G/A
7-2: 55966729C/A
7-2: 56015563C/T
7-2: 56050823C/T
7-2: 56099549T/G
7-2: 56180577T/G
7-2: 56207978T/C
7-2: 56233557C/T
7-2: 56303993T/C
7-2: 56349516T/A
7-2: 56425057C/T
7-2: 56466115A/G
7-2: 56482278C/T
7-2: 56521039A/G
7-2: 56541098G/A
7-2: 56560767G/A
7-2: 56588516A/G
7-2: 56664926G/A
7-2: 56736099C/T
7-2: 56761323G/A
7-2: 56795940G/T
7-2: 56817957A/C
7-2: 56836855T/C
7-2: 56861924T/C
7-2: 56915569T/C
7-2: 56933086A/C
7-2: 56969613A/T
7-2: 56993522C/T
7-2: 57046285C/T
7-2: 57078252A/G
7-2: 57184800A/T
7-2: 57249590C/T
7-2: 57315868G/T
7-2: 57387511G/A
7-2: 57531561A/G
7-2: 57601679A/C
7-2: 57603615C/T
7-2: 57635365T/C
7-2: 57675329T/A
7-2: 57686963T/C
7-2: 57726444C/A
7-2: 57772200G/A
7-2: 57813564A/G
7-2: 57858225G/A
7-2: 57867835G/A
7-2: 57895198T/C
7-2: 57907852G/C
7-2: 57990826C/A
7-2: 58034461C/T
7-2: 58045230T/C
7-2: 58051391C/T
7-2: 58140012A/C
7-2: 58194949C/T
7-2: 58260042A/T
7-2: 58291551A/G
7-2: 58306907G/A
7-2: 58319723T/G
7-2: 58336281T/C
7-2: 58348497T/A
7-2: 58375806T/C
7-2: 58390008G/A
7-2: 58406893A/T
7-2: 58411497A/G
7-2: 58422609G/A
7-2: 58438311T/G
7-2: 58467822G/A
7-2: 58474855C/T
7-2: 58506226G/C
7-2: 58535231T/C
7-2: 58645759C/T
7-2: 58648277T/A
7-2: 58695804G/C
7-2: 58777859A/T
7-2: 58791074C/T
7-2: 58810943C/T
7-2: 58845561A/T
7-2: 58913641C/T
7-2: 59026591G/C
7-2: 59277378A/G
7-2: 59348493T/C
7-2: 59387278C/G
7-2: 59409927C/T
7-2: 59469464G/T
7-2: 59469866T/A
7-2: 59510143G/A
7-2: 59784957G/C
7-2: 59786393C/A
7-2: 59805567A/T
7-2: 59826841C/G
7-2: 59856987A/T
7-2: 59966295C/T
7-2: 59981912A/G
7-2: 60105233G/A
7-2: 60135732A/G
7-2: 60233578G/A
7-2: 60499724T/C
7-2: 60526377G/A
7-2: 60554278T/C
7-2: 60572330T/C
7-2: 60651299T/A
7-2: 60751038A/G
7-2: 60850481G/A
7-2: 60982749T/C
7-2: 60992862A/T
7-2: 61244312T/G
7-2: 61331694A/C
7-2: 61353445C/T
7-2: 61461720A/G
7-2: 61461825A/G
7-2: 61461920A/T
7-2: 61483206A/T
7-2: 61508704T/C
7-2: 61521394A/G
7-2: 61549165G/A
7-2: 61574608T/C
7-2: 61593919C/T
7-2: 61621068C/G
7-2: 61738519T/C
7-2: 61816261A/G
7-2: 61853514C/T
7-2: 61984239T/C
7-2: 62008493A/T
7-2: 62081791C/T
7-2: 62108769C/G
7-2: 62125172C/T
7-2: 62135649G/A
7-2: 62247216C/G
7-2: 62267619G/C
7-2: 62295575G/C
7-2: 62348185C/T
7-2: 62355873T/C
7-2: 62355878G/A
7-2: 62371378A/G
7-2: 62387492A/G
7-2: 62423930C/T
7-2: 62494645G/A
7-2: 62499591C/A
7-2: 62814014C/T
7-2: 62860500G/C
7-2: 62902892T/C
7-2: 62943074T/G
7-2: 62955589C/T
7-2: 63010364G/A
7-2: 63089351G/A
7-2: 63131661T/G
7-2: 63137930C/T
7-2: 63139651G/A
7-2: 63139866A/G
7-2: 63140447A/G
7-2: 63154749G/C
7-2: 63187401C/T
7-2: 63220853G/A
7-2: 63237867C/G
7-2: 63278246A/G
7-2: 63278773C/A
7-2: 63394572A/T
7-2: 63409061C/T
7-2: 63429899T/A
7-2: 63472128G/A
7-2: 63598623C/T
7-2: 63607397G/A
7-2: 63631854T/A
7-2: 63668857A/G
7-2: 63700837C/T
7-2: 63758705A/G
7-2: 63848437C/T
7-2: 63885605T/C
7-2: 63942146G/C
7-2: 64036652T/A
7-2: 64086784A/G
7-2: 64140482A/C
7-2: 64140890C/G
7-2: 64224919T/G
7-2: 64286426C/G
7-2: 64361685G/A
7-2: 64406193G/A
7-2: 64436451T/C
7-2: 64453398G/A
7-2: 64471124C/A
7-2: 64527997A/G
7-2: 64564622A/G
7-2: 64581614C/T
7-2: 64657234A/G
7-2: 64657644G/A
7-2: 64657746G/A
7-2: 64657767A/G
7-2: 64658307C/T
7-2: 64658814G/A
7-2: 64658921T/C
7-2: 64659060C/T
7-2: 64659086G/A
7-2: 64661661G/T
7-2: 64705015A/T
7-2: 64733992T/A
7-2: 64755610C/A
7-2: 64761761A/C
7-2: 64777060G/A
7-2: 64835064C/T
7-2: 64862887T/G
7-2: 64947780A/C
7-2: 64955794T/C
7-2: 65020657C/A
7-2: 65053450T/G
7-2: 65083447T/C
7-2: 65083500C/G
7-2: 65107434A/C
7-2: 65182778C/T
7-2: 65213347G/A
7-2: 65409804C/T
7-2: 65544213T/C
7-2: 65555801G/A
7-2: 65588943A/T
7-2: 65599819T/A
7-2: 65623439T/G
7-2: 65623503T/C
7-2: 65623647A/G
7-2: 65624964G/A
7-2: 65651945T/G
7-2: 65664678A/G
7-2: 65688140T/A
7-2: 65734918G/A
7-2: 65771835A/G
7-2: 65836168C/G
7-2: 65911563T/G
7-2: 65943232G/A
7-2: 65989845T/.
7-2: 66012151T/C
7-2: 66044293G/A
7-2: 66092804C/G
7-2: 66093914G/C
7-2: 66136516G/A
7-2: 66145792T/A
7-2: 66145809T/G
7-2: 66145876C/T
7-2: 66232899C/G
7-2: 66256680C/T
7-2: 66318096T/C
7-2: 66351191G/T
7-2: 66377328T/C
7-2: 66383166T/C
7-2: 66400650G/A
7-2: 66400671C/T
7-2: 66444419C/T
7-2: 66502733A/G
7-2: 66571798T/A
7-2: 66603630A/.
7-2: 66610614T/.
7-2: 66610650A/.
7-2: 66610661A/.
7-2: 66610676T/.
7-2: 66610801T/.
7-2: 66610802G/.
7-2: 66624780C/G
7-2: 66711949T/C
7-2: 66802944G/A
7-2: 66842990T/C
7-2: 66875494C/A
7-2: 66876912G/T
7-2: 66890802A/G
7-2: 66901760A/T
7-2: 66912046T/A
7-2: 66922955C/T
7-2: 66978628C/T
7-2: 66988637A/T
7-2: 66989038T/C
7-2: 66989172C/G
7-2: 66989930T/C
7-2: 66990964T/C
7-2: 66996937G/T
7-2: 66997057C/A
7-2: 66997408T/A
7-2: 66998118T/A
7-2: 66998130A/G
7-2: 67001753T/A
7-2: 67002330A/G
7-2: 67002518A/G
7-2: 67002767G/C
7-2: 67028765G/A
7-2: 67061116T/C
7-2: 67100714T/C
7-2: 67141046G/A
7-2: 67180033C/T
7-2: 67239580G/A
7-2: 67251548C/T
7-2: 67271568C/T
7-2: 67280029A/G
7-2: 67316074A/T
7-2: 67338265T/A
7-2: 67349076G/T
7-2: 67363071A/C
7-2: 67417276C/A
7-2: 67610476T/C
7-2: 67704052T/A
7-2: 67716740G/A
7-2: 67769228A/G
7-2: 67832564T/C
7-2: 67847932A/T
7-2: 67875353G/A
7-2: 68004345T/C
7-2: 68008235C/A
7-2: 68124084A/G
7-2: 68125662T/C
7-2: 68335045C/T
7-2: 68354603A/T
7-2: 68395232T/A
7-2: 68432830T/A
7-2: 68510140G/A
7-2: 68580218G/C
7-2: 68587045T/A
7-2: 68609623T/A
7-2: 68655232A/G
7-2: 68662904G/A
7-2: 68666955G/A
7-2: 68673571C/T
7-2: 68729418A/G
7-2: 68790787G/A
7-2: 68860446A/T
7-2: 69014477G/A
7-2: 69087380A/C
7-2: 69135679T/C
7-2: 69157887G/A
7-2: 69242237T/C
7-2: 69293742A/G
7-2: 69313202G/T
7-2: 69317350G/A
7-2: 69325105T/C
7-2: 69336114C/A
7-2: 69429175G/A
7-2: 69471971T/C
7-2: 69550673G/A
7-2: 69561339T/A
7-2: 69564118G/T
7-2: 69564501G/A
7-2: 69585684C/A
7-2: 69596556A/T
7-2: 69609166G/C
7-2: 69638734G/A
7-2: 69659999T/A
7-2: 69758474C/A
7-2: 69779406T/A
7-2: 69806203C/T
7-2: 69806227A/T
7-2: 69807126T/A
7-2: 69894417C/T
7-2: 69912505C/G
7-2: 69923409T/C
7-2: 70016014C/T
7-2: 70040064A/G
7-2: 70082917A/G
7-2: 70132048T/A
7-2: 70145044C/T
7-2: 70183600G/C
7-2: 70215411A/G
7-2: 70248388C/A
7-2: 70305023G/T
7-2: 70318225C/A
7-2: 70444748T/C
7-2: 70456200T/G
7-2: 70473962C/T
7-2: 70524701G/A
7-2: 70584015T/C
7-2: 70596884T/C
7-2: 70737506A/G
7-2: 70759731A/C
7-2: 70857337G/A
7-2: 70884425T/A
7-2: 70954735T/C
7-2: 70975425A/G
7-2: 70984875A/T
7-2: 70994046A/T
7-2: 71002114A/G
7-2: 71006800G/A
7-2: 71016893A/G
7-2: 71051657T/C
7-2: 71074376A/G
7-2: 71124090G/T
7-2: 71192726G/A
7-2: 71212131G/T
7-2: 71223101A/G
7-2: 71281682G/A
7-2: 71311689G/A
7-2: 71442281C/G
7-2: 71462532G/A
7-2: 71501485G/C
7-2: 71501568G/A
7-2: 71531225G/A
7-2: 71558293T/C
7-2: 71585509A/G
7-2: 71625362C/T
7-2: 71705652G/A
7-2: 71786088G/C
7-2: 71813910G/A
7-2: 71893391T/G
7-2: 71895129T/G
7-2: 71896167T/G
7-2: 71937743T/A
7-2: 71988399G/A
7-2: 72046975C/T
7-2: 72082251A/G
7-2: 72112880A/G
7-2: 72135286G/A
7-2: 72219712C/A
7-2: 72248499A/G
7-2: 72325084C/A
7-2: 72342047A/C
7-2: 72405396C/T
7-2: 72425340G/A
7-2: 72451092C/G
7-2: 72486233C/A
7-2: 72530251C/T
7-2: 72560034T/C
7-2: 72561563C/G
7-2: 72634608T/C
7-2: 72741537T/C
7-2: 72772293G/A
7-2: 72803821G/A
7-2: 72815218G/A
7-2: 72829559T/C
7-2: 72837271C/T
7-2: 72876093T/C
7-2: 72887696A/T
7-2: 72905090A/G
7-2: 72924895G/T
7-2: 72935253T/C
7-2: 72973293T/A
7-2: 73008359C/G
7-2: 73043408C/T
7-2: 73088163C/A
7-2: 73146994T/A
7-2: 73214058G/A
7-2: 73271349A/C
7-2: 73281538T/C
7-2: 73292217T/G
7-2: 73293070T/C
7-2: 73302955C/A
7-2: 73331234G/A
7-2: 73426392T/A
7-2: 73437426G/A
7-2: 73437486C/T
7-2: 73515244C/A
7-2: 73551719T/C
7-2: 73585642G/T
7-2: 73616503C/T
7-2: 73704665A/T
7-2: 73738937C/T
7-2: 73784016C/T
7-2: 73898477C/T
7-2: 73912237T/G
7-2: 73967707T/C
7-2: 74060150C/T
7-2: 74120645T/C
7-2: 74157166T/C
7-2: 74283449A/C
7-2: 74404609T/A
7-2: 74429971T/C
7-2: 74475387C/T
7-2: 74513685A/G
7-2: 74570318T/A
7-2: 74635946C/A
7-2: 74686646T/C
7-2: 74780526C/G
7-2: 74900172C/T
7-2: 74901804G/A
7-2: 74953428T/A
7-2: 75045654T/C
7-2: 75142172G/A
7-2: 75197367A/C
7-2: 75239892G/A
7-2: 75273175C/T
7-2: 75342626A/G
7-2: 75405385C/T
7-2: 75638552T/C
7-2: 75650861A/G
7-2: 75661930C/T
7-2: 75826769A/G
7-2: 75861906C/G
7-2: 75932750T/A
7-2: 75943948T/G
7-2: 75991481A/G
7-2: 76051516G/A
7-2: 76159782C/T
7-2: 76233148A/G
7-2: 76343787T/G
7-2: 76412373C/T
7-2: 76453461T/A
7-2: 76453760G/T
7-2: 76461878A/C
7-2: 76461908A/G
7-2: 76462564G/A
7-2: 76463093T/C
7-2: 76463632C/A
7-2: 76519066T/G
7-2: 76662378C/A
7-2: 76662516C/T
7-2: 76819554T/A
7-2: 76836511G/A
7-2: 76863051G/C
7-2: 77009826C/T
7-2: 77052088C/A
7-2: 77163887A/C
7-2: 77165016C/A
7-2: 77202773C/T
7-2: 77241834A/G
7-2: 77301123C/T
7-2: 77336052T/C
7-2: 77336187G/C
7-2: 77362521A/T
7-2: 77399966C/T
7-2: 77461663T/C
7-2: 77582636C/A
7-2: 77639379C/A
7-2: 77835062C/A
7-2: 77854745T/C
7-2: 78043674A/G
7-2: 78055873G/A
7-2: 78056343G/A
7-2: 78105366C/T
7-2: 78142564C/T
7-2: 78184244G/A
7-2: 78255827A/T
7-2: 78502276A/T
7-2: 78516718C/T
7-2: 78638174A/G
7-2: 78719111T/G
7-2: 78805081T/C
7-2: 78864554A/T
7-2: 78879979C/T
7-2: 78895911C/A
7-2: 78923516C/A
7-2: 78955398A/C
7-2: 78993257G/T
7-2: 79032693C/T
7-2: 79068839T/C
7-2: 79189287G/A
7-2: 79202531T/A
7-2: 79210975A/C
7-2: 79224610A/G
7-2: 79267377T/C
7-2: 79459337G/T
7-2: 79467325T/C
7-2: 79785619C/T
7-2: 79965489G/A
7-2: 79976750A/C
7-2: 80001936T/A
7-2: 80050495C/A
7-2: 80080949T/C
7-2: 80111716A/G
7-2: 80229879T/G
7-2: 80318261A/G
7-2: 80336000A/T
7-2: 80481679A/G
7-2: 80495997A/G
7-2: 80510072G/A
7-2: 80660390C/T
7-2: 80672154G/A
7-2: 80764720T/G
7-2: 80905269T/C
7-2: 80960560T/C
7-2: 81124243C/T
7-2: 81189502A/G
7-2: 81446093G/A
7-2: 81490947A/C
7-2: 81570370C/T
7-2: 81625074G/A
7-2: 81688583A/T
7-2: 81742261C/T
7-2: 81873405T/G
7-2: 81876016G/A
7-2: 81893221T/A
7-2: 81986631A/G
7-2: 82055993A/G
7-2: 82116708T/C
7-2: 82203483C/T
7-2: 82248948A/T
7-2: 82274591G/A
7-2: 82392112C/T
7-2: 82427868A/T
7-2: 82454846T/C
7-2: 82537670C/T
7-2: 82664162A/G
7-2: 82769499C/T
7-2: 82848660T/A
7-2: 82988693T/C
7-2: 82989811A/T
7-2: 82989848C/G
7-2: 82989889A/G
7-2: 82989909A/T
7-2: 83102188A/G
7-2: 83277021G/C
7-2: 83295005T/G
7-2: 83344636G/A
7-2: 83382409G/A
7-2: 83438481G/A
7-2: 83537060G/T
7-2: 83581574C/G
7-2: 83683599T/C
7-2: 83929043T/C
7-2: 84094049G/A
7-2: 84145559A/G
7-2: 84146705A/G
7-2: 84158678A/G
7-2: 84174912A/T
7-2: 84185978A/T
7-2: 84206743G/A
7-2: 84245437A/T
7-2: 84261332C/T
7-2: 84353763G/T
7-2: 84391790T/C
7-2: 84442000A/C
7-2: 84480410C/A
7-2: 84766491A/G
7-2: 85173784A/T
7-2: 85217986A/C
7-2: 85316650C/T
7-2: 85604503G/C
7-2: 85701471C/T
7-2: 85771637T/C
7-2: 85908771A/G
7-2: 85926511A/G
7-2: 85975382G/A
7-2: 86076912C/G
7-2: 86289052T/G
7-2: 86332442A/G
7-2: 86370172A/G
7-2: 86463901T/C
7-2: 86481763T/C
7-2: 86499666C/T
7-2: 86527173G/A
7-2: 86648634A/G
7-2: 86753111G/A
7-2: 86988015A/G
7-2: 86996681C/A
7-2: 86996954C/A
7-2: 86996983A/G
7-2: 86998568A/G
7-2: 86998635G/T
7-2: 86998742G/T
7-2: 86998843A/G
7-2: 87002739A/T
7-2: 87823333G/A
7-2: 87983115G/A
7-2: 87993162G/A
7-3: 75255A/T
7-3: 86185C/G
7-3: 87239T/C
7-3: 241768T/A
7-3: 314544C/T
7-3: 360594C/T
7-3: 561131A/G
7-3: 789048G/T
7-3: 1076313G/A
7-3: 1150832G/C
7-3: 1202719A/G
7-3: 1292767A/C
7-3: 1408389C/A
7-3: 1624949C/T
7-3: 1953881A/G
7-3: 2008426G/T
7-3: 2009673G/A
7-3: 2114755T/C
7-3: 2119200C/T
7-3: 2122117A/G
7-3: 2354261C/T
7-3: 2357417C/A
7-3: 2401259G/A
7-3: 2513993G/C
7-3: 2635897C/T
7-3: 2922685A/G
7-3: 2974106G/A
7-3: 2991807C/T
7-3: 3060411A/T
7-3: 3115736A/G
7-3: 3165926T/A
7-3: 3372611A/G
7-3: 3474385C/G
7-3: 3483252T/A
7-3: 3498204C/A
7-3: 3699075C/T
7-3: 3709186G/A
7-3: 3720107T/C
7-3: 3774616A/T
7-3: 3792003T/C
7-3: 3812486T/G
7-3: 4012718G/A
7-3: 4014737C/T
7-3: 4615321T/C
7-3: 4674742A/G
7-3: 4723673A/G
7-3: 4758063G/C
7-3: 4811451G/A
7-3: 4828333T/C
7-3: 4839701G/T
7-3: 4854195G/A
7-3: 5255042G/A
7-3: 5297676T/A
7-3: 5338236G/T
7-3: 5572995C/A
7-3: 5726120A/T
7-3: 5815855G/C
7-3: 5880267C/T
7-3: 5947223A/T
7-3: 5989196T/C
7-3: 6026874C/T
7-3: 6087368G/A
7-3: 6141371G/A
7-3: 6244676C/T
7-3: 6486379C/A
7-3: 6510214A/T
7-3: 6522855T/A
7-3: 6589646A/G
7-3: 6626576C/T
7-3: 6643340C/T
7-3: 6683754G/T
7-3: 6753375G/A
7-3: 6782241G/T
7-3: 6828985T/A
7-3: 6920784C/T
7-3: 7009576T/G
7-3: 7062004A/G
7-3: 7091706C/T
7-3: 7107497T/C
7-3: 7125864C/T
7-3: 7147138A/G
7-3: 7189030T/A
7-3: 7191734A/T
7-3: 7202008T/A
7-3: 7202886T/C
7-3: 7215026A/G
7-3: 7265164G/C
7-3: 7273902A/T
7-3: 7278811T/A
7-3: 7278920A/G
7-3: 7298853C/T
7-3: 7360727T/G
7-3: 7361810C/G
7-3: 7367378G/C
7-3: 7387210G/A
7-3: 7450992G/T
7-3: 7477926C/T
7-3: 7478168A/G
7-3: 7510351G/A
7-3: 7545144T/C
7-3: 7628333G/C
7-3: 7699335C/T
7-3: 7746175C/T
7-3: 7833234C/A
7-3: 7996375G/T
7-3: 8011761T/A
7-3: 8072629G/T
7-3: 8091894C/G
7-3: 8254338T/C
7-3: 8258390A/G
7-3: 8265904C/T
7-3: 8288950G/A
7-3: 8301270G/A
7-3: 8304026A/C
7-3: 8331876G/T
7-3: 8355458A/G
7-3: 8384803A/C
7-3: 8430208T/C
7-3: 8469701A/G
7-3: 8475302G/A
7-3: 8475988T/A
7-3: 8476218G/A
7-3: 8476328G/A
7-3: 8484526C/T
7-3: 8484569G/A
7-3: 8485717A/T
7-3: 8486664C/A
7-3: 8521155G/A
7-3: 8521355A/G
7-3: 8523530A/G
7-3: 8554792C/T
7-3: 8592002G/A
7-3: 8620911T/C
7-3: 8660887A/G
7-3: 8735637T/C
7-3: 8854299C/T
7-3: 8860080T/C
7-3: 8862185C/T
7-3: 8863012T/G
7-3: 8865912C/G
7-3: 8898235T/C
7-3: 8961620C/T
7-3: 8972360G/A
7-3: 8991679A/T
7-3: 8992078G/C
7-3: 9086138T/C
7-3: 9119857G/A
7-3: 9158702C/A
7-3: 9160634G/A
7-3: 9169812A/G
7-3: 9190694T/A
7-3: 9216329A/C
7-3: 9230700A/G
7-3: 9266902A/G
7-3: 9319434T/G
7-3: 9352858G/A
7-3: 9407759C/A
7-3: 9420492T/C
7-3: 9435014A/G
7-3: 9457730G/A
7-3: 9575609A/T
7-3: 9656926G/A
7-3: 9867063A/G
7-3: 9876499G/A
7-3: 9910865A/T
7-3: 9916917C/A
7-3: 9959956T/C
7-3: 9969605G/A
7-3: 9970989A/G
7-3: 10002345A/G
7-3: 10367102C/T
7-3: 10515389C/T
7-3: 10627772C/A
7-3: 10630388T/C
7-3: 10649554G/A
7-3: 10736015A/G
7-3: 10736375T/G
7-3: 10919373A/G
7-3: 10939808T/C
7-3: 11076915C/T
7-3: 11178841G/C
7-3: 11325724A/T
7-3: 11488942C/T
7-3: 11512837C/T
7-3: 11565185G/C
7-3: 11602866G/T
7-3: 11690545A/C
7-3: 11778838C/T
7-3: 11811682G/A
7-3: 11823303T/A
7-3: 11841380C/T
7-3: 11855514A/T
7-3: 11866169C/T
7-3: 11930455G/A
7-3: 11995247T/A
7-3: 12005562C/T
7-3: 12009589C/T
7-3: 12053384T/C
7-3: 12131485T/C
7-3: 12141925G/C
7-3: 12142840T/G
7-3: 12256532C/T
7-3: 12266776G/A
7-3: 12283581G/A
7-3: 12301094T/C
7-3: 12349821A/G
7-3: 12375274C/T
7-3: 12386115C/T
7-3: 12391150T/C
7-3: 12397426C/A
7-3: 12430751A/T
7-3: 12433490C/T
7-3: 12433657A/G
7-3: 12433808C/T
7-3: 12433813T/C
7-3: 12435069G/A
7-3: 12435148C/T
7-3: 12435283A/T
7-3: 12435456T/C
7-3: 12437426C/T
7-3: 12438473G/T
7-3: 12447768C/G
7-3: 12448222A/T
7-3: 12448313T/C
7-3: 12448366T/C
7-3: 12468375G/A
7-3: 12606677A/T
7-3: 12618608C/T
7-3: 12632429G/A
7-3: 12649766A/C
7-3: 12651858C/T
7-3: 12652034C/A
7-3: 12653374A/T
7-3: 12666224T/A
7-3: 12667613C/T
7-3: 12681828C/G
7-3: 12695610G/A
7-3: 12696684A/G
7-3: 12711264A/G
7-3: 12726477T/A
7-3: 12737267T/C
7-3: 12742260G/A
7-3: 12749367A/G
7-3: 12772078A/G
7-3: 12810727C/A
7-3: 12857944G/A
7-3: 12925127G/A
7-3: 12950189G/A
7-3: 12980389T/C
7-3: 12992001C/A
7-3: 13012202G/A
7-3: 13045564G/A
7-3: 13045688G/A
7-3: 13045889G/T
7-3: 13046051G/A
7-3: 13056791T/G
7-3: 13076279A/G
7-3: 13081687T/C
7-3: 13081792A/T
7-3: 13084664T/C
7-3: 13087454C/T
7-3: 13087802T/C
7-3: 13087814C/T
7-3: 13088813C/T
7-3: 13096853C/G
7-3: 13097652T/C
7-3: 13118266G/T
7-3: 13127171G/T
7-3: 13127861T/G
7-3: 13129457G/C
7-3: 13129982A/G
7-3: 13130473A/G
7-3: 13176364C/T
7-3: 13213554G/T
7-3: 13227640A/G
7-3: 13237959G/C
7-3: 13238116C/A
7-3: 13238227T/A
7-3: 13238977G/T
7-3: 13337784A/C
7-3: 13396795A/C
7-3: 13586736A/G
7-3: 13621723G/A
7-3: 13647759A/G
7-3: 13663099C/A
7-3: 13705795C/G
7-3: 13719781T/C
7-3: 13722833T/C
7-3: 13733064A/G
7-3: 13758463T/C
7-3: 13769366G/A
7-3: 13813924A/G
7-3: 13820524C/T
7-3: 13820954G/A
7-3: 13821632T/C
7-3: 13824152G/A
7-3: 13864183C/T
7-3: 13933540T/A
7-3: 13944611T/C
7-3: 14034410T/C
7-3: 14047101G/T
7-3: 14071998A/T
7-3: 14101249A/G
7-3: 14149512A/G
7-3: 14164590G/A
7-3: 14183112A/G
7-3: 14202010G/A
7-3: 14250584A/T
7-3: 14294464C/G
7-3: 14312745G/A
7-3: 14331886A/C
7-3: 14362987G/A
7-3: 14407903T/C
7-3: 14559087C/T
7-3: 14564336A/T
7-3: 14564449G/A
7-3: 14564707A/G
7-3: 14565175G/C
7-3: 14565392A/C
7-3: 14565395A/C
7-3: 14566096T/G
7-3: 14566582T/C
7-3: 14566604C/T
7-3: 14567103C/T
7-3: 14567132T/C
7-3: 14567301C/A
7-3: 14569934T/A
7-3: 14571863T/A
7-3: 14572552A/C
7-3: 14644666A/G
7-3: 14655583G/A
7-3: 14686083C/T
7-3: 14692304A/G
7-3: 14696086C/T
7-3: 14738520A/G
7-3: 14781950A/G
7-3: 14801788G/A
7-3: 14802955A/G
7-3: 14810546G/A
7-3: 14811065C/T
7-3: 14811090T/G
7-3: 14811805A/T
7-3: 14829516T/C
7-3: 14829711G/A
7-3: 14848536G/A
7-3: 14854933A/G
7-3: 14855661G/A
7-3: 14858582G/A
7-3: 14869526C/G
7-3: 14898096G/A
7-3: 14950611G/A
7-3: 14973577G/A
7-3: 14991161A/C
7-3: 15003196G/A
7-3: 15003368A/C
7-3: 15003532C/T
7-3: 15005694C/T
7-3: 15007737G/A
7-3: 15066158T/G
7-3: 15119232C/G
7-3: 15158722A/C
7-3: 15169142C/T
7-3: 15177911T/C
7-3: 15231582A/T
7-3: 15240686A/T
7-3: 15271756A/T
7-3: 15272532A/T
7-3: 15272556T/C
7-3: 15290039A/C
7-3: 15330792A/G
7-3: 15349338C/T
7-3: 15367428C/G
7-3: 15410498C/T
7-3: 15424400C/T
7-3: 15439447G/A
7-3: 15453701C/G
7-3: 15471487T/C
7-3: 15482450C/T
7-3: 15493598T/A
7-3: 15511868T/A
7-3: 15512293T/G
7-3: 15528384C/T
7-3: 15541791C/T
7-3: 15553520C/T
7-3: 15559244A/T
7-3: 15560579C/A
7-3: 15560716C/T
7-3: 15568206C/G
7-3: 15580326G/A
7-3: 15591662C/T
7-3: 15633246C/T
7-3: 15666697G/A
7-3: 15678924A/C
7-3: 15722663T/A
7-3: 15748254A/G
7-3: 15780887G/A
7-3: 15799529C/T
7-3: 15802446C/T
7-3: 15821631T/C
7-3: 15855390G/A
7-3: 15870369G/T
7-3: 15884170A/C
7-3: 15884425T/C
7-3: 15885523C/T
7-3: 15920100C/T
7-3: 15924822A/G
7-3: 15937479A/T
7-3: 15968594A/T
7-3: 16001949G/A
7-3: 16018542C/T
7-3: 16061617A/G
7-3: 16110548A/T
7-3: 16199579C/T
7-3: 16221098G/A
7-3: 16275508C/T
7-3: 16377771C/A
7-3: 16380205A/G
7-3: 16395089T/C
7-3: 16426830A/G
7-3: 16504855T/C
7-3: 16554157G/A
7-3: 16596508G/A
7-3: 16608084C/T
7-3: 16701785G/A
7-3: 16702039T/G
7-3: 16828053G/A
7-3: 16829308G/T
7-3: 16862918A/G
7-3: 16874362C/T
7-3: 16874381T/A
7-3: 16876011T/C
7-3: 16881242T/C
7-3: 16881964C/T
7-3: 16882069C/T
7-3: 16882303T/A
7-3: 16885994T/C
7-3: 16891460A/T
7-3: 16983373C/T
7-3: 16993889T/A
7-3: 17016919A/G
7-3: 17030549G/C
7-3: 17092608C/T
7-3: 17093177G/T
7-3: 17124018T/C
7-3: 17167327A/C
7-3: 17194739A/G
7-3: 17205947C/T
7-3: 17284320C/A
7-3: 17305748A/G
7-3: 17316911G/T
7-3: 17335357G/A
7-3: 17519797C/T
7-3: 17683887T/C
7-3: 17689739C/T
7-3: 17719118T/A
7-3: 17745367T/G
7-3: 17821965G/C
7-3: 17885591T/C
7-3: 17904039A/G
7-3: 17922531A/T
7-3: 17934413G/T
7-3: 17963835C/T
7-3: 18071088T/C
7-3: 18072555C/T
7-3: 18121217T/C
7-3: 18189438T/C
7-3: 18227322C/A
7-3: 18308579C/A
7-3: 18321827A/G
7-3: 18346814C/G
7-3: 18360942C/T
7-3: 18403945G/A
7-3: 18440473T/C
7-3: 18456259T/G
7-3: 18469888A/C
7-3: 18483342G/T
7-3: 18499911A/C
7-3: 18520285G/A
7-3: 18543788A/G
7-3: 18584900C/T
7-3: 18630046A/G
7-3: 18630160A/T
7-3: 18630879T/C
7-3: 18631209G/A
7-3: 18644155A/G
7-3: 18697671C/G
7-3: 18757803C/T
7-3: 18771092C/T
7-3: 18808642A/G
7-3: 18847181T/C
7-3: 18880122T/C
7-3: 18942952G/A
7-3: 19071204T/C
7-3: 19106951T/C
7-3: 19122529T/C
7-3: 19172204T/A
7-3: 19241910T/C
7-3: 19290268A/C
7-3: 19356754T/C
7-3: 19492611G/T
7-3: 19540931C/T
7-3: 19567542A/T
7-3: 19703024G/A
7-3: 19714444C/A
7-3: 19731419G/T
7-3: 19751409C/T
7-3: 19778814G/A
7-3: 19807579C/T
7-3: 19846338C/T
7-3: 19893127A/G
7-3: 19990216C/A
7-3: 20068799G/A
7-3: 20109061A/G
7-3: 20185348A/G
7-3: 20242751A/G
7-3: 20282083A/G
7-3: 20311339A/T
7-3: 20345546A/G
7-3: 20410277A/T
7-3: 20457248A/G
7-3: 20498356G/T
7-3: 20531419T/A
7-3: 20596044A/T
7-3: 20640804A/T
7-3: 20671329T/A
7-3: 20704930G/C
7-3: 20721719G/A
7-3: 20746728C/T
7-3: 20802939A/G
7-3: 20806904A/G
7-3: 20807114C/T
7-3: 20807151C/A
7-3: 20822563G/A
7-3: 20891753G/A
7-3: 21127783G/C
7-3: 21266469T/C
7-3: 21315000C/T
7-3: 21335941C/T
7-3: 21534891A/G
7-3: 21540501C/T
7-3: 21569927A/T
7-3: 21658364A/G
7-3: 21685483G/A
7-3: 21734277G/A
7-3: 21928256T/C
7-3: 21928752C/T
7-3: 21928793T/C
7-3: 21928794T/C
7-3: 21928859G/C
7-3: 21929005G/T
7-3: 21930004G/A
7-3: 21930259C/T
7-3: 21930517A/T
7-3: 21930545A/G
7-3: 21964775T/G
7-3: 22004565A/G
7-3: 22043656T/A
7-3: 22055531T/A
7-3: 22101991A/T
7-3: 22174787A/C
7-3: 22255765A/G
7-3: 22331877C/T
7-3: 22397433G/T
7-3: 22432411A/T
7-3: 22433778C/T
7-3: 22434315T/C
7-3: 22466791A/G
7-3: 22507091A/G
7-3: 22562353G/A
7-3: 22568893C/T
7-3: 22581054G/A
7-3: 22584257G/A
7-3: 22586446T/C
7-3: 22586747T/A
7-3: 22586859C/T
7-3: 22587414C/T
7-3: 22587526T/G
7-3: 22587634C/T
7-3: 22594063A/G
7-3: 22595150T/A
7-3: 22595157T/G
7-3: 22607107G/A
7-3: 22607176T/A
7-3: 22607250T/G
7-3: 22607563C/A
7-3: 22608307C/A
7-3: 22637658T/G
7-3: 22640300C/A
7-3: 22640412A/G
7-3: 22640534C/T
7-3: 22641002C/A
7-3: 22642031G/A
7-3: 22642048A/T
7-3: 22642569T/G
7-3: 22665112A/G
7-3: 22670810G/T
7-3: 22671380T/C
7-3: 22671493A/G
7-3: 22678074T/G
7-3: 22695458A/T
7-3: 22702243C/T
7-3: 22702348T/G
7-3: 22714025T/A
7-3: 22736944A/G
7-3: 22741277C/T
7-3: 22741645T/C
7-3: 22755584G/T
7-3: 22755936A/T
7-3: 22756330G/T
7-3: 22756352A/G
7-3: 22756730T/A
7-3: 22756833A/G
7-3: 22757726C/T
7-3: 22767324G/A
7-3: 22781512A/G
7-3: 22797238C/T
7-3: 22854879T/C
7-3: 22855150C/A
7-3: 22857314C/T
7-3: 22940263C/T
7-3: 23004052G/C
7-3: 23054053T/G
7-3: 23102112A/T
7-3: 23148414T/G
7-3: 23192908A/T
7-3: 23328987C/A
7-3: 23376760A/G
7-3: 23408915G/A
7-3: 23444974T/C
7-3: 23477068C/T
7-3: 23508750C/T
7-3: 23538808A/G
7-3: 23698406T/C
7-3: 23701861A/G
7-3: 23737315G/T
7-3: 23777921C/T
7-3: 23861185A/T
7-3: 23866757C/T
7-3: 23874793A/T
7-3: 23889724T/G
7-3: 23916910C/A
7-3: 23966582A/T
7-3: 23999622T/C
7-3: 24013673A/G
7-3: 24051306T/C
7-3: 24072645T/A
7-3: 24106693G/T
7-3: 24155661G/T
7-3: 24203857A/G
7-3: 24230335G/A
7-3: 24245366T/C
7-3: 24257192A/C
7-3: 24292966T/A
7-3: 24354426A/C
7-3: 24400566T/C
7-3: 24401187G/A
7-3: 24401289G/A
7-3: 24402877G/A
7-3: 24402918G/T
7-3: 24402947C/T
7-3: 24403447C/T
7-3: 24403523G/A
7-3: 24403621T/C
7-3: 24403729A/G
7-3: 24403817C/A
7-3: 24429820G/T
7-3: 24438897C/T
7-3: 24438918T/G
7-3: 24458504C/T
7-3: 24496074A/G
7-3: 24531604G/C
7-3: 24606604A/T
7-3: 24639169G/T
7-3: 24679743A/C
7-3: 24759719T/A
7-3: 24793103T/C
7-3: 24904194A/G
7-3: 24939688T/C
7-3: 24996042G/A
7-3: 25113113G/A
7-3: 25113192T/G
7-3: 25134279T/A
7-3: 25134308T/A
7-3: 25180130C/T
7-3: 25271784A/C
7-3: 25286151G/A
7-3: 25453159T/A
7-3: 25541929C/A
7-3: 25543584A/G
7-3: 25629138T/C
7-3: 25629984A/G
7-3: 25824154G/C
7-3: 25837196T/C
7-3: 25862530T/G
7-3: 25933033G/A
7-3: 26072104A/T
7-3: 26171755T/A
7-3: 26199611G/C
7-3: 26327068C/T
7-3: 26340596T/A
7-3: 26342292C/T
7-3: 26342595G/A
7-3: 26345949T/C
7-3: 26347234T/C
7-3: 26348859G/T
7-3: 26349048G/A
7-3: 26349216G/T
7-3: 26349322G/A
7-3: 26349347G/A
7-3: 26350970C/T
7-3: 26375190C/A
7-3: 26376345G/T
7-3: 26376377T/A
7-3: 26386269C/T
7-3: 26437303A/T
7-3: 26491210A/G
7-3: 26493727C/T
7-3: 26496919G/A
7-3: 26523328T/G
7-3: 26557964C/T
7-3: 26638981G/A
7-3: 26650652T/A
7-3: 26668369A/G
7-3: 26680539G/A
7-3: 26688400C/T
7-3: 26691550T/C
7-3: 26710284T/C
7-3: 26724711A/G
7-3: 26752718G/A
7-3: 26752901T/C
7-3: 26752909T/A
7-3: 26794689G/A
7-3: 26855891C/T
7-3: 26894250T/C
7-3: 26926214G/C
7-3: 27009462C/T
7-3: 27027015C/T
7-3: 27110975G/A
7-3: 27168786G/A
7-3: 27197642G/C
7-3: 27262518A/G
7-3: 27262794C/T
7-3: 27278773A/T
7-3: 27307333A/C
7-3: 27370192G/A
7-3: 27423400A/C
7-3: 27488931T/A
7-3: 27498671C/G
7-3: 27510614A/C
7-3: 27531792C/T
7-3: 27570073G/T
7-3: 27603072A/G
7-3: 27614501A/G
7-3: 27625305A/G
7-3: 27656113A/G
7-3: 27721493T/A
7-3: 27737020G/T
7-3: 27835003G/T
7-3: 27861693A/G
7-3: 27863854A/G
7-3: 27887932G/C
7-3: 27904135G/A
7-3: 27953329T/C
7-3: 27990659C/G
7-3: 28011606C/A
7-3: 28047745G/A
7-3: 28169420G/A
7-3: 28174592A/T
7-3: 28239897A/T
7-3: 28240333T/C
7-3: 28254140G/A
7-3: 28288681C/T
7-3: 28315004A/C
7-3: 28315110G/T
7-3: 28331740G/A
7-3: 28332433C/G
7-3: 28364835G/C
7-3: 28392143T/C
7-3: 28456623A/G
7-3: 28483121C/T
7-3: 28591233C/T
7-3: 28591617G/A
7-3: 28606834T/C
7-3: 28625026T/C
7-3: 28635751T/A
7-3: 28727297T/C
7-3: 28800933G/T
7-3: 28835760C/T
7-3: 28867492A/G
7-3: 29061706T/C
7-3: 29178840G/A
7-3: 29195182C/T
7-3: 29214431G/A
7-3: 29284480C/T
7-3: 29325219A/G
7-3: 29364655A/C
7-3: 29371235T/A
7-3: 29491193G/A
7-3: 29498837C/T
7-3: 29527394A/G
7-3: 29570983C/T
7-3: 29582633T/A
7-3: 29583897A/G
7-3: 29598832T/C
7-3: 29630755A/G
7-3: 29643255T/G
7-3: 29708368G/C
7-3: 29770534A/C
7-3: 29796070G/C
7-3: 29816292T/A
7-3: 29823699A/G
7-3: 29852185T/G
7-3: 29887992T/C
7-3: 29939755C/T
7-3: 29949252A/G
7-3: 29949366A/C
7-3: 29949494T/G
7-3: 29949596A/G
7-3: 29949711T/C
7-3: 29949863C/T
7-3: 29950008T/C
7-3: 29950164A/T
7-3: 29992873C/T
7-3: 30007366G/A
7-3: 30067567A/T
7-3: 30077988A/T
7-3: 30131244A/T
7-3: 30169914T/A
7-3: 30217368C/T
7-3: 30304335C/G
7-3: 30316344A/T
7-3: 30378555A/T
7-3: 30453266C/T
7-3: 30464453T/C
7-3: 30619238G/A
7-3: 30629902G/C
7-3: 30659555T/C
7-3: 30690826G/C
7-3: 30743128G/A
7-3: 30842205C/T
7-3: 30901319A/G
7-3: 30908021C/T
7-3: 30952192G/C
7-3: 30979589T/C
7-3: 31017053A/C
7-3: 31052523T/A
7-3: 31073230G/A
7-3: 31114578A/T
7-3: 31150170A/G
7-3: 31173886A/G
7-3: 31264670A/G
7-3: 31282773G/A
7-3: 31363141A/G
7-3: 31435510T/A
7-3: 31546640G/A
7-3: 31655547G/A
7-3: 31692102T/C
7-3: 31763134A/T
7-3: 31828015A/T
7-3: 31918493T/C
7-3: 31969329A/G
7-3: 32108750A/T
7-3: 32183363C/T
7-3: 32222139T/C
7-3: 32392492A/G
7-3: 32471918C/T
7-3: 32500955C/T
7-3: 32511554T/C
7-3: 32562471G/T
7-3: 32833246G/A
7-3: 32848950A/G
7-3: 32867593A/T
7-3: 32889488T/C
7-3: 32889515C/T
7-3: 32901288G/T
7-3: 32990002T/C
7-3: 33216805C/T
7-3: 33225633T/G
7-3: 33248631A/T
7-3: 33260692A/G
7-3: 33282760A/T
7-3: 33294539G/A
7-3: 33294673A/C
7-3: 33335472A/G
7-3: 33381677C/G
7-3: 33438711C/G
7-3: 33439518A/C
7-3: 33452831G/A
7-3: 33480020T/G
7-3: 33510995A/T
7-3: 33555677C/T
7-3: 33593012C/T
7-3: 33619457A/G
7-3: 33694486G/T
7-3: 33727977G/A
7-3: 33756972C/T
7-3: 33819638C/T
7-3: 33850623G/A
7-3: 33893825T/C
7-3: 33905886T/G
7-3: 33947962G/T
7-3: 33967615A/G
7-3: 33979992T/C
7-3: 34011534A/G
7-3: 34051979G/A
7-3: 34052419T/C
7-3: 34052674T/C
7-3: 34055718C/T
7-3: 34055757A/T
7-3: 34064660C/A
7-3: 34078338G/A
7-3: 34104759T/G
7-3: 34107097T/C
7-3: 34158714A/G
7-3: 34172529G/A
7-3: 34202006G/T
7-3: 34212267A/G
7-3: 34230964G/T
7-3: 34244389C/T
7-3: 34254598T/A
7-3: 34261165G/A
7-3: 34261166G/T
7-3: 34262585T/A
7-3: 34263030G/A
7-3: 34263045G/A
7-3: 34265201C/T
7-3: 34279883A/G
7-3: 34322884A/C
7-3: 34342971A/G
7-3: 34349620A/T
7-3: 34380387A/G
7-3: 34404214A/G
7-3: 34538815A/T
7-3: 34605820T/C
7-3: 34625341G/A
7-3: 34627978A/T
7-3: 34644995T/G
7-3: 34647852G/A
7-3: 34662974C/T
7-3: 34675822C/A
7-3: 34691325T/A
7-3: 34700338A/T
7-3: 34716626A/G
7-3: 34750255C/T
7-3: 34750596G/A
7-3: 34759168T/A
7-3: 34759303C/T
7-3: 34760623C/T
7-3: 34766749A/G
7-3: 34779673C/A
7-3: 34806283G/A
7-3: 34811451T/C
7-3: 34817140A/G
7-3: 34843268A/G
7-3: 34935170T/C
7-3: 35024832T/C
7-3: 35208605G/T
7-3: 35228394G/A
7-3: 35276026T/C
7-3: 35289604T/C
7-3: 35301641C/T
7-3: 35321189G/A
7-3: 35336473G/A
7-3: 35383103T/C
7-3: 35432474T/C
7-3: 35448097C/G
7-3: 35476985T/C
7-3: 35502291C/T
7-3: 35533307T/C
7-3: 35617452T/G
7-3: 35661264A/G
7-3: 35663768G/A
7-3: 35777814A/G
7-3: 35777931T/G
7-3: 35786359G/A
7-3: 35796720C/T
7-3: 35796950C/T
7-3: 35913190C/A
7-3: 35952119T/A
7-3: 36126640C/T
7-3: 36126657C/T
7-3: 36137134A/T
7-3: 36382438C/A
7-3: 36537793A/C
7-3: 36637564A/G
7-3: 36684301A/G
7-3: 36685759A/G
7-3: 36745542A/G
7-3: 36795620A/G
7-3: 36843054T/C
7-3: 36896592C/T
7-3: 36933933T/A
7-3: 36955594G/A
7-3: 36998735T/C
7-3: 37097165A/C
7-3: 37108640T/C
7-3: 37143905C/T
7-3: 37218751T/C
7-3: 37219649T/A
7-3: 37235280A/T
7-3: 37263618G/A
7-3: 37310512T/C
7-3: 37416340G/A
7-3: 37428709A/G
7-3: 37448215T/C
7-3: 37539869A/G
7-3: 37553187G/A
7-3: 37558807T/C
7-3: 37563227T/A
7-3: 37611540A/G
7-3: 37659905G/A
7-3: 37671642G/A
7-3: 37785107C/T
7-3: 37833242T/G
7-3: 37856498A/G
7-3: 37882096T/G
7-3: 37882647G/C
7-3: 37882868A/G
7-3: 37896314C/T
7-3: 37909895C/T
7-3: 37939489C/T
7-3: 38002955C/T
7-3: 38158771T/G
7-3: 38196648C/T
7-3: 38196692C/A
7-3: 38196754T/G
7-3: 38196907G/T
7-3: 38208982A/C
7-3: 38224875A/G
7-3: 38258174C/T
7-3: 38264988G/C
7-3: 38265092G/A
7-3: 38265186T/C
7-3: 38265499A/G
7-3: 38265518C/T
7-3: 38273062A/T
7-3: 38284119C/T
7-3: 38284420A/T
7-3: 38378543G/A
7-3: 38422523C/T
7-3: 38530798A/G
7-3: 38575921G/A
7-3: 38593276G/A
7-3: 38603565C/T
7-3: 38626151A/G
7-3: 38661069T/C
7-3: 38674451A/T
7-3: 38686403G/A
7-3: 38704500G/A
7-3: 38718846C/T
7-3: 38731931G/A
7-3: 38762353G/C
7-3: 38785154T/C
7-3: 38785155G/A
7-3: 38785164C/A
7-3: 38785171T/C
7-3: 38785197T/A
7-3: 38785203C/T
7-3: 38785207A/G
7-3: 38785222G/T
7-3: 38785231T/C
7-3: 38785248T/C
7-3: 38785274T/A
7-3: 38785279C/T
7-3: 38785287C/G
7-3: 38785312C/T
7-3: 38785335G/A
7-3: 38785354C/A
7-3: 38785371T/C
7-3: 38785399T/A
7-3: 38785448C/T
7-3: 38785472T/C
7-3: 38785473G/A
7-3: 38811973C/T
7-3: 38842725C/A
7-3: 38865761C/A
7-3: 38865765G/T
7-3: 38869150C/T
7-3: 38885998G/C
7-3: 38969953A/T
7-3: 39013267A/T
7-3: 39029980C/T
7-3: 39049723G/A
7-3: 39060069A/G
7-3: 39091846G/T
7-3: 39127139A/C
7-3: 39156357T/C
7-3: 39172829T/C
7-3: 39183674G/A
7-3: 39199145G/A
7-3: 39226727A/G
7-3: 39242728A/C
7-3: 39286500C/T
7-3: 39287769A/G
7-3: 39306062T/A
7-3: 39329591G/A
7-3: 39349743G/A
7-3: 39355227A/G
7-3: 39361727G/C
7-3: 39377457C/T
7-3: 39389447A/G
7-3: 39391854T/C
7-3: 39394617T/C
7-3: 39394618C/T
7-3: 39394658G/A
7-3: 39394723G/A
7-3: 39394825G/A
7-3: 39400745A/G
7-3: 39414077A/G
7-3: 39414215T/A
7-3: 39417472T/A
7-3: 39423461C/T
7-3: 39445317G/A
7-3: 39533011A/C
7-3: 39558165G/A
7-3: 39595204G/A
7-3: 39596845T/C
7-3: 39616371G/A
7-3: 39643441A/G
7-3: 39726716T/C
7-3: 39734961T/G
7-3: 39737286G/A
7-3: 39740522C/A
7-3: 39741408C/T
7-3: 39752226C/T
7-3: 39752564G/A
7-3: 39752577A/G
7-3: 39775193C/T
7-3: 39793889A/G
7-3: 39849647G/A
7-3: 39859954T/C
7-3: 39903082G/A
7-3: 39940315A/G
7-3: 40006715T/C
7-3: 40039622C/G
7-3: 40131418G/A
7-3: 40174205A/G
7-3: 40212078T/A
7-3: 40233130A/G
7-3: 40264174T/G
7-3: 40287315A/C
7-3: 40327121A/C
7-3: 40327206C/T
7-3: 40403812G/T
7-3: 40439320C/G
7-3: 40458055A/C
7-3: 40481210C/T
7-3: 40492660T/C
7-3: 40514674C/T
7-3: 40517262A/G
7-3: 40531423T/C
7-3: 40535138G/A
7-3: 40538756C/A
7-3: 40538900A/G
7-3: 40539156A/G
7-3: 40539571A/G
7-3: 40540373A/T
7-3: 40541085T/C
7-3: 40541838A/T
7-3: 40553392T/C
7-3: 40563962A/G
7-3: 40575756C/T
7-3: 40580652C/G
7-3: 40581275C/T
7-3: 40583487C/T
7-3: 40604707C/T
7-3: 40605138C/T
7-3: 40605145C/T
7-3: 40611006G/T
7-3: 40617598C/T
7-3: 40644460G/A
7-3: 40657575T/C
7-3: 40716018T/G
7-3: 40727977T/A
7-3: 40818273T/A
7-3: 40830745A/C
7-3: 40853146C/A
7-3: 40857641A/G
7-3: 40867224C/T
7-3: 40967643T/G
7-3: 40976832T/C
7-3: 41053031G/C
7-3: 41053718A/C
7-3: 41054421A/G
7-3: 41087228T/C
7-3: 41191838C/T
7-3: 41219228G/A
7-3: 41261596T/C
7-3: 41302674C/T
7-3: 41354179C/T
7-3: 41366726T/C
7-3: 41383479C/T
7-3: 41396284A/C
7-3: 41406315C/A
7-3: 41424744G/A
7-3: 41450191C/T
7-3: 41496963T/G
7-3: 41527733C/T
7-3: 41547948T/G
7-3: 41610672T/G
7-3: 41618004C/T
7-3: 41624510A/T
7-3: 41624647C/G
7-3: 41645366A/T
7-3: 41696645G/T
7-3: 41731850G/A
7-3: 41766697T/C
7-3: 41991761C/T
7-3: 42038617A/G
7-3: 42074777C/T
7-3: 42153179T/C
7-3: 42176102A/G
7-3: 42199208G/A
7-3: 42304664A/G
7-3: 42327310A/G
7-3: 42345791G/A
7-3: 42361684A/G
7-3: 42372405G/A
7-3: 42530114A/G
7-3: 42530221A/C
7-3: 42530445C/A
7-3: 42579658T/A
7-3: 42612425C/T
7-3: 42639601C/G
7-3: 42670285C/T
7-3: 42682973G/A
7-3: 42685151G/A
7-3: 42690992C/A
7-3: 42714426T/C
7-3: 42736446T/C
7-3: 42743795C/T
7-3: 42765336G/A
7-3: 42767005G/T
7-3: 42767148T/C
7-3: 42767220A/C
7-3: 42767418T/G
7-3: 42767432A/G
7-3: 42767549G/A
7-3: 42767840A/G
7-3: 42775857G/C
7-3: 42782246C/A
7-3: 42789631T/A
7-3: 42817157G/A
7-3: 42840496C/T
7-3: 42865494C/T
7-3: 42893226A/T
7-3: 42924470A/G
7-3: 42960775G/A
7-3: 43014939A/G
7-3: 43040393T/C
7-3: 43054374G/A
7-3: 43059094G/T
7-3: 43072656C/G
7-3: 43075973A/T
7-3: 43075999C/A
7-3: 43231277A/G
7-3: 43231303C/A
7-3: 43231480G/A
7-3: 43231593A/G
7-3: 43231636A/C
7-3: 43231647A/G
7-3: 43233492A/G
7-3: 43253101C/T
7-3: 43264134T/A
7-3: 43264266T/A
7-3: 43264368T/C
7-3: 43266043G/A
7-3: 43266118T/A
7-3: 43274221G/A
7-3: 43274225A/G
7-3: 43275868T/G
7-3: 43276828T/C
7-3: 43282212G/A
7-3: 43282236C/T
7-3: 43282403T/C
7-3: 43282506G/A
7-3: 43282578T/C
7-3: 43282648C/T
7-3: 43282731T/C
7-3: 43288754A/C
7-3: 43290428C/T
7-3: 43290529G/A
7-3: 43292419T/G
7-3: 43371309A/G
7-3: 43447473C/A
7-3: 43510032A/C
7-3: 43577300G/A
7-3: 43577348C/A
7-3: 43640110T/C
7-3: 43693624A/T
7-3: 43704559C/T
7-3: 43714468A/T
7-3: 43714469T/C
7-3: 43728187C/T
7-3: 43743483C/A
7-3: 43743491G/A
7-3: 43743755G/A
7-3: 43743990G/A
7-3: 43842752A/T
7-3: 43855440A/C
7-3: 43872678T/A
7-3: 43879754C/T
7-3: 43879873G/A
7-3: 43882800G/T
7-3: 43893016G/A
7-3: 43903908A/T
7-3: 43926302T/C
7-3: 43983744G/A
7-3: 44002382C/T
7-3: 44011348C/G
7-3: 44035309G/A
7-3: 44045056A/T
7-3: 44096894T/C
7-3: 44126538A/T
7-3: 44227159T/G
7-3: 44230594T/C
7-3: 44345715A/G
7-3: 44385071A/G
7-3: 44501446C/G
7-3: 44609366T/C
7-3: 44615432C/T
7-3: 44626334G/T
7-3: 44628666T/C
7-3: 44663505G/A
7-3: 44689711G/A
7-3: 44723468T/C
7-3: 44754323C/T
7-3: 44798089A/T
7-3: 44877649C/A
7-3: 45039415A/T
7-3: 45107623A/G
7-3: 45192540C/A
7-3: 45235983C/A
7-3: 45283703C/T
7-3: 45357074A/T
7-3: 45360502A/T
7-3: 45360581A/G
7-3: 45360713G/T
7-3: 45360725T/.
7-3: 45360743T/.
7-3: 45360819T/.
7-3: 45360860G/A
7-3: 45360892T/.
7-3: 45360907C/T
7-3: 45360970T/.
7-3: 45361044T/C
7-3: 45367984T/G
7-3: 45384366C/T
7-3: 45431996G/T
7-3: 45469479T/C
7-3: 45538505C/T
7-3: 45707489A/T
7-3: 45738989T/C
7-3: 45864230G/A
7-3: 45896036A/T
7-3: 45922582C/A
7-3: 45947993C/A
7-3: 45974129G/A
7-3: 45995264G/T
7-3: 45997893G/A
7-3: 46011239T/A
7-3: 46036837G/C
7-3: 46140721A/G
7-3: 46305415A/T
7-3: 46308776A/C
7-3: 46321925A/T
7-3: 46327732T/C
7-3: 46334576T/C
7-3: 46334907T/G
7-3: 46362281A/C
7-3: 46425833C/T
7-3: 46427778G/A
7-3: 46427789T/A
7-3: 46455603A/C
7-3: 46458231G/T
7-3: 46513250A/G
7-3: 46531431T/A
7-3: 46578721T/G
7-3: 46641101T/A
7-3: 46678505G/T
7-3: 46718648G/A
7-3: 46767407T/A
7-3: 46770606G/C
7-3: 46770663T/A
7-3: 46794630T/C
7-3: 46844388A/G
7-3: 46869508G/A
7-3: 47062865C/G
7-3: 47213164T/C
7-3: 47244071A/G
7-3: 47272646T/C
7-3: 47366152T/C
7-3: 47436911G/A
7-3: 47462300G/A
7-3: 47495724T/C
7-3: 47521070T/C
7-3: 47560105G/T
7-3: 47583606C/T
7-3: 47611156C/A
7-3: 47624628C/A
7-3: 47651321A/C
7-3: 47687245T/C
7-3: 47713692G/A
7-3: 47728737T/C
7-3: 47728879C/T
7-3: 47782724C/T
7-3: 47820401A/T
7-3: 47874536C/T
7-3: 47884548A/G
7-3: 47925894A/G
7-3: 47977144A/C
7-3: 47977442C/T
7-3: 47977543G/A
7-3: 47977659G/T
7-3: 47977663C/T
7-3: 47979551G/A
7-3: 47979708G/A
7-3: 47980001A/C
7-3: 47980034G/C
7-3: 47980192C/T
7-3: 47980294T/C
7-3: 47980892G/C
7-3: 47980952A/G
7-3: 47980966G/A
7-3: 47980999C/T
7-3: 47995833A/C
7-3: 48081683C/T
7-3: 48193022C/T
7-3: 48199353G/A
7-3: 48203804C/T
7-3: 48203825C/T
7-3: 48211925A/T
7-3: 48245917G/A
7-3: 48375215G/A
7-3: 48386194C/T
7-3: 48423445A/C
7-3: 48469575C/A
7-3: 48480364T/C
7-3: 48511883A/T
7-3: 48554881A/T
7-3: 48576734C/T
7-3: 48602273A/G
7-3: 48636227T/A
7-3: 48656182G/T
7-3: 48675257C/T
7-3: 48743687T/C
7-3: 48798185C/T
7-3: 48808712C/T
7-3: 48825996G/A
7-3: 48883586T/C
7-3: 48895943G/T
7-3: 48913535C/T
7-3: 48984882C/G
7-3: 48995375A/G
7-3: 49005879A/G
7-3: 49022142C/T
7-3: 49099093C/T
7-3: 49154432T/C
7-3: 49213745G/C
7-3: 49227398C/A
7-3: 49241554A/G
7-3: 49251894C/T
7-3: 49271501G/T
7-3: 49320990A/C
7-3: 49363202G/T
7-3: 49389471C/T
7-3: 49514230G/A
7-3: 49582123G/A
7-3: 49625547C/A
7-3: 49685657G/A
7-3: 49754062G/A
7-3: 49846561G/A
7-3: 49887722A/T
7-3: 49888701T/C
7-3: 49963689T/C
7-3: 49993592T/G
7-3: 50036135A/T
7-3: 50037292A/T
7-3: 50037313A/T
7-3: 50037550T/G
7-3: 50038867T/G
7-3: 50039153G/A
7-3: 50039264G/A
7-3: 50039380G/A
7-3: 50040420C/T
7-3: 50040532T/C
7-3: 50040928G/A
7-3: 50041499G/A
7-3: 50041509T/C
7-3: 50041952G/T
7-3: 50042033A/T
7-3: 50042059C/T
7-3: 50042160A/G
7-3: 50042341T/A
7-3: 50042442G/T
7-3: 50042650C/A
7-3: 50042687T/C
7-3: 50047171A/G
7-3: 50047838T/A
7-3: 50048315A/C
7-3: 50048558C/T
7-3: 50048560A/G
7-3: 50048873A/C
7-3: 50052410C/T
7-3: 50052556T/C
7-3: 50052703G/T
7-3: 50052813T/C
7-3: 50052816T/C
7-3: 50052996C/T
7-3: 50061235A/C
7-3: 50061486C/A
7-3: 50061585A/G
7-3: 50093163A/G
7-3: 50107374A/G
7-3: 50109109A/T
7-3: 50109121A/C
7-3: 50109323T/C
7-3: 50109436G/A
7-3: 50109621T/A
7-3: 50109644G/T
7-3: 50109807C/A
7-3: 50110037G/C
7-3: 50110077C/A
7-3: 50110122G/T
7-3: 50110674A/C
7-3: 50112945C/T
7-3: 50112949T/C
7-3: 50146716G/A
7-3: 50148798C/A
7-3: 50165346A/T
7-3: 50166862A/G
7-3: 50167440T/A
7-3: 50167727G/T
7-3: 50167854A/G
7-3: 50167944A/T
7-3: 50168194G/A
7-3: 50168356A/C
7-3: 50168492C/A
7-3: 50168627G/A
7-3: 50168765C/G
7-3: 50168853C/T
7-3: 50169488C/A
7-3: 50169566C/G
7-3: 50266310A/G
7-3: 50267136A/T
7-3: 50267801C/T
7-3: 50311932A/G
7-3: 50329057G/A
7-3: 50346706G/T
7-3: 50347526C/A
7-3: 50415329A/G
7-3: 50446520T/A
7-3: 50480684C/T
7-3: 50505460A/G
7-3: 50517202A/G
7-3: 50565151G/A
7-3: 50575715T/G
7-3: 50615789A/G
7-3: 50624699C/A
7-3: 50643064C/T
7-3: 50682692G/A
7-3: 50762834C/T
7-3: 50817534A/T
7-3: 50828539A/G
7-3: 50974167C/T
7-3: 50996589G/A
7-3: 51007064T/A
7-3: 51010661C/A
7-3: 51011181A/T
7-3: 51088247G/A
7-3: 51132256C/T
7-3: 51200493A/G
7-3: 51295986T/A
7-3: 51315233A/T
7-3: 51323892A/C
7-3: 51382910G/T
7-3: 51408826G/A
7-3: 51436681A/C
7-3: 51450080G/T
7-3: 51505200C/T
7-3: 51515830G/A
7-3: 51521099T/C
7-3: 51553124A/G
7-3: 51582394G/A
7-3: 51648144T/G
7-3: 51723677G/A
7-3: 51817387C/G
7-3: 51854979G/A
7-3: 51905086A/G
7-3: 51984349T/C
7-3: 52009294C/T
7-3: 52014067G/A
7-3: 52024223A/G
7-3: 52028201C/T
7-3: 52055938T/A
7-3: 52070920A/G
7-3: 52084597C/T
7-3: 52102190A/G
7-3: 52102730T/C
7-3: 52103197G/A
7-3: 52103255T/C
7-3: 52119323G/A
7-3: 52122638C/T
7-3: 52122793A/T
7-3: 52131998G/A
7-3: 52147866A/T
7-3: 52154138C/A
7-3: 52155700C/A
7-3: 52155726G/A
7-3: 52209841T/A
7-3: 52245800T/A
7-3: 52278591G/A
7-3: 52330149G/A
7-3: 52411305A/G
7-3: 52459298C/T
7-3: 52484229A/G
7-3: 52575816T/A
7-3: 52614303T/A
7-3: 52757841T/C
7-3: 52803872G/A
7-3: 52821759G/T
7-3: 52828667G/A
7-3: 52832371G/A
7-3: 52833084T/C
7-3: 52877802G/A
7-3: 52888508G/A
7-3: 52908746A/G
7-3: 52921356C/A
7-3: 52928471G/A
7-3: 52939326C/T
7-3: 52968293C/T
7-3: 53038694T/A
7-3: 53065535C/T
7-3: 53090690A/G
7-3: 53090993T/A
7-3: 53091017T/C
7-3: 53171490T/C
7-3: 53198047T/A
7-3: 53268109C/T
7-3: 53375258C/T
7-3: 53377715A/G
7-3: 53445888A/T
7-3: 53492753G/A
7-3: 53710434A/T
7-3: 53837565A/C
7-3: 53967275G/A
7-3: 54005010T/C
7-3: 54028906A/G
7-3: 54059714C/T
7-3: 54060109C/G
7-3: 54064254C/T
7-3: 54064895G/A
7-3: 54064925G/T
7-3: 54070400C/T
7-3: 54082726T/C
7-3: 54112860A/G
7-3: 54155117A/G
7-3: 54169549T/G
7-3: 54185017G/T
7-3: 54272169C/T
7-3: 54287687G/A
7-3: 54300929C/T
7-3: 54306733T/A
7-3: 54307783T/G
7-3: 54307828A/T
7-3: 54307932G/A
7-3: 54336018C/T
7-3: 54350082G/A
7-3: 54359656A/C
7-3: 54456878A/G
7-3: 54538139A/C
7-3: 54593986A/T
7-3: 54627475C/A
7-3: 54629907G/C
7-3: 54630693T/C
7-3: 54655331C/T
7-3: 54675842G/A
7-3: 54676667T/C
7-3: 54676778A/C
7-3: 54676805G/A
7-3: 54705270T/C
7-3: 54716098C/T
7-3: 54716938G/A
7-3: 54720298A/T
7-3: 54731561A/G
7-3: 54744083G/A
7-3: 54744195T/C
7-3: 54744323T/G
7-3: 54745946C/A
7-3: 54748676C/T
7-3: 54751977C/T
7-3: 54765989C/T
7-3: 54768263G/A
7-3: 54768482T/A
7-3: 54774327G/A
7-3: 54778308T/G
7-3: 54816500T/G
7-3: 54848124C/T
7-3: 54928166C/T
7-3: 55017453T/A
7-3: 55093414C/A
7-3: 55114859G/A
7-3: 55140560T/C
7-3: 55146441A/G
7-3: 55146443G/A
7-3: 55146805A/T
7-3: 55146836C/G
7-3: 55146842T/A
7-3: 55147780A/G
7-3: 55147784A/G
7-3: 55148046A/C
7-3: 55328682G/C
7-3: 55456755A/G
7-3: 55696570T/A
7-3: 55780758A/G
7-3: 55953158G/A
7-3: 55967406A/T
7-3: 56032931G/T
7-3: 56087225C/T
7-3: 56099959C/T
7-3: 56100029A/C
7-3: 56113957A/C
7-3: 56182487C/T
7-3: 56210467T/C
7-3: 56210600A/T
7-3: 56326218C/T
7-3: 56380707A/G
7-3: 56385658T/C
7-3: 56416727C/A
7-3: 56444845G/A
7-3: 56484383C/T
7-3: 56503539T/C
7-3: 56508203T/C
7-3: 56544807T/A
7-3: 56577020G/A
7-3: 56612077C/T
7-3: 56674750C/T
7-3: 56773226G/A
7-3: 56919339C/T
7-3: 56924321T/A
7-3: 56956571A/T
7-3: 57041048A/G
7-3: 57077620T/C
7-3: 57114998C/G
7-3: 57127974T/C
7-3: 57153749G/A
7-3: 57171884C/G
7-3: 57220761A/G
7-3: 57261430T/C
7-3: 57359552C/A
7-3: 57404729C/T
7-3: 57447675T/C
7-3: 57452456C/T
7-3: 57476507A/T
7-3: 57513657G/A
7-3: 57543653T/C
7-3: 57545488G/A
7-3: 57545596A/G
7-3: 57545864C/G
7-3: 57545977G/A
7-3: 57547010A/T
7-3: 57548070T/A
7-3: 57569918T/C
7-3: 57601603T/G
7-3: 57604571T/A
7-3: 57664063C/A
7-3: 57710626T/A
7-3: 57763943C/T
7-3: 57826895G/A
7-3: 57871772T/A
7-3: 57961345T/A
7-3: 57975908T/C
7-3: 58058049A/T
7-3: 58227697C/T
7-3: 58292506A/G
7-3: 58328039A/T
7-3: 58377648A/T
7-3: 58406722A/C
7-3: 58426151C/A
7-3: 58513391G/A
7-3: 58514446A/G
7-3: 58519570C/T
7-3: 58539308G/A
7-3: 58539773T/C
7-3: 58542266T/C
7-3: 58542672C/T
7-3: 58543799A/G
7-3: 58544687T/C
7-3: 58544720A/C
7-3: 58564243T/C
7-3: 58577701T/C
7-3: 58588361C/A
7-3: 58588647G/A
7-3: 58589046A/G
7-3: 58589228C/A
7-3: 58593783A/G
7-3: 58599136T/C
7-3: 58618163G/A
7-3: 58630173T/G
7-3: 58630879G/T
7-3: 58631041T/G
7-3: 58640973T/C
7-3: 58641287A/G
7-3: 58647183A/G
7-3: 58663004A/G
7-3: 58672643T/C
7-3: 58673440G/A
7-3: 58688179A/T
7-3: 58708795C/T
7-3: 58719416T/G
7-3: 58753574C/A
7-3: 58904970G/A
7-3: 58910588A/G
7-3: 58947141A/G
7-3: 59005212A/G
7-3: 59074484T/C
7-3: 59098568A/C
7-3: 59123614G/A
7-3: 59167868G/T
7-3: 59196603A/T
7-3: 59220737C/G
7-3: 59245357G/A
7-3: 59251291T/G
7-3: 59270157A/C
7-3: 59284431A/G
7-3: 59382215A/G
7-3: 59450830C/T
7-3: 59461832T/C
7-3: 59484575C/G
7-3: 59508512T/C
7-3: 59513231A/C
7-3: 59535613C/A
7-3: 59562489G/A
7-3: 59754970C/G
7-3: 59754989C/T
7-3: 59755019T/C
7-3: 59977942C/T
7-3: 60029373C/T
7-3: 60031401A/C
7-3: 60128035G/A
7-3: 60188789A/G
7-3: 60309861C/A
7-3: 60515419G/A
7-3: 60594826G/A
7-3: 60633441A/T
7-3: 60687710G/A
7-3: 60737774T/A
7-3: 60876701G/A
7-3: 60887955A/G
7-3: 60955942C/A
7-3: 61028352G/A
7-3: 61061017C/T
7-3: 61085282G/A
7-3: 61085387C/A
7-3: 61089124A/G
7-3: 61115681T/C
7-3: 61115783T/A
7-3: 61130720T/G
7-3: 61214773G/A
7-3: 61259541G/T
7-3: 61425816C/A
7-3: 61437252A/G
7-3: 61490319A/G
7-3: 61519904C/G
7-3: 61559182G/T
7-3: 61569628T/C
7-3: 61659899G/A
7-3: 61723124A/G
7-3: 61769792A/G
7-3: 61777039T/A
7-3: 61785878T/C
7-3: 61817681C/T
7-3: 61817917A/G
7-3: 61818235T/C
7-3: 61853877A/T
7-3: 61868966T/C
7-3: 61921227G/A
7-3: 62015479C/T
7-3: 62026834G/A
7-3: 62056882T/C
7-3: 62114891T/A
7-3: 62205394T/C
7-3: 62231954T/A
7-3: 62278506C/A
7-3: 62317580G/A
7-3: 62342526C/T
7-3: 62400917T/C
7-3: 62414284G/A
7-3: 62486565T/G
7-3: 62522083C/T
7-3: 62602094A/G
7-3: 62694768G/C
7-3: 62732784C/T
7-3: 62786639A/G
7-3: 62818911A/T
7-3: 62855668T/C
7-3: 62870513C/T
7-3: 62874542C/T
7-3: 62874827C/T
7-3: 62876559G/T
7-3: 62884774C/T
7-3: 62951449G/C
7-3: 62951837A/G
7-3: 62980982G/A
7-3: 63052747T/G
7-3: 63063122T/A
7-3: 63063371T/C
7-3: 63068692A/T
7-3: 63069038G/A
7-3: 63069314C/T
7-3: 63071507G/A
7-3: 63071913C/A
7-3: 63072019G/C
7-3: 63072023T/C
7-3: 63083515T/C
7-3: 63098486G/T
7-3: 63099458T/G
7-3: 63113122C/A
7-3: 63114603G/T
7-3: 63116730G/C
7-3: 63116738A/T
7-3: 63123361G/A
7-3: 63135838C/A
7-3: 63193359T/G
7-3: 63223780C/T
7-3: 63263709G/C
7-3: 63317965T/A
7-3: 63390095G/A
7-3: 63416792C/T
7-3: 63455722A/G
7-3: 63597355A/G
7-3: 63640314G/T
7-3: 63727672T/C
7-3: 63814179T/G
7-3: 63893116T/G
7-3: 63938277C/T
7-3: 63948614A/G
7-3: 64040998G/C
7-3: 64073045G/A
7-3: 64178588T/C
7-3: 64224534C/T
7-3: 64266728G/T
7-3: 64313679C/T
7-3: 64368042T/C
7-3: 64507996G/A
7-3: 64580107G/A
7-3: 64602674C/T
7-3: 64727505T/C
7-3: 64756205T/C
7-3: 64790976A/G
7-3: 64873856G/A
7-3: 64989498G/T
7-3: 65188919A/G
7-3: 65191332C/T
7-3: 65203017T/G
7-3: 65337042A/C
7-3: 65366304G/A
7-3: 65381487G/C
7-3: 65460513A/G
7-3: 65529801A/T
7-3: 65637884A/G
7-3: 65678949A/G
7-3: 65716682C/T
7-3: 65809257A/T
7-3: 65949502A/G
7-3: 66059875T/C
7-3: 66129844A/T
7-3: 66204522A/C
7-3: 66236563T/A
7-3: 66251029C/T
7-3: 66287751G/A
7-3: 66371480T/C
7-3: 66543981G/C
7-3: 66614188C/T
7-3: 66658898T/C
7-3: 66767088A/G
7-3: 66850473T/C
7-3: 66892548G/C
7-3: 66981266C/A
7-3: 67113668T/G
7-3: 67113776G/C
7-3: 67162278A/C
7-3: 67265056A/T
7-3: 67294792C/T
7-3: 67364797C/T
7-3: 67474796G/T
7-3: 67504319A/T
7-3: 67544685G/A
7-3: 67621596T/C
7-3: 67671089A/G
7-3: 67681665C/T
7-3: 67719416A/T
7-3: 67843178T/C
7-3: 67846617A/G
7-3: 67911699C/T
7-3: 68089185A/G
7-3: 68114233A/T
7-3: 68127950C/A
7-3: 68219684T/A
7-3: 68301812C/G
7-3: 68432215G/T
7-3: 68432393A/T
7-3: 68433155G/T
7-3: 68433516T/C
7-3: 68433717C/T
7-3: 68434228T/C
7-3: 68434357T/G
7-3: 68434478A/G
7-3: 68435223T/G
7-3: 68451845G/C
7-3: 68466753T/C
7-3: 68488206C/T
7-3: 68488223C/T
7-3: 68488238G/A
7-3: 68582751C/G
7-3: 68642187T/A
7-3: 68674927C/T
7-3: 68741780A/T
7-3: 68829669C/T
7-3: 68863583T/C
7-3: 68905862G/T
7-3: 68906449G/A
7-3: 68906554G/A
7-3: 68907345A/G
7-3: 68907985G/C
7-3: 68909943G/T
7-3: 68910527T/C
7-3: 68912446C/T
7-3: 68912653T/C
7-3: 68913124A/C
7-3: 68914840C/T
7-3: 68916375C/T
7-3: 68980299C/T
7-3: 69043317C/G
7-3: 69073374A/G
7-3: 69248489T/A
7-3: 69309490C/T
7-3: 69395065A/G
7-3: 69499233G/T
7-3: 69549029C/G
7-3: 69578510C/A
7-3: 69653158T/G
7-3: 69784556T/C
7-3: 69806850C/T
7-3: 69826411A/G
7-3: 69879146C/T
7-3: 69984284A/C
7-3: 70010888T/C
7-3: 70061568C/T
7-3: 70065349C/T
7-3: 70081655A/G
7-3: 70083136G/A
7-3: 70083199G/A
7-3: 70083285T/A
7-3: 70083433A/G
7-3: 70143251T/C
7-3: 70154423A/G
7-3: 70251106G/A
7-3: 70259138A/G
7-3: 70270289T/C
7-3: 70272521C/T
7-3: 70272633G/T
7-3: 70276273A/T
7-3: 70276436A/T
7-3: 70276535A/G
7-3: 70276601C/A
7-3: 70276626T/C
7-3: 70276668A/G
7-3: 70276907C/T
7-3: 70277207A/T
7-3: 70277309G/T
7-3: 70277412G/A
7-3: 70280554G/A
7-3: 70295427C/G
7-3: 70311180G/A
7-3: 70312655A/G
7-3: 70312758G/A
7-3: 70313163T/C
7-3: 70375246C/T
7-3: 70402511C/T
7-3: 70435304A/G
7-3: 70499687T/C
7-3: 70549527A/G
7-3: 70601053C/T
7-3: 70629115G/T
7-3: 70651667A/G
7-3: 70653733A/C
7-3: 70694245T/C
7-3: 70696793G/A
7-3: 70697024A/T
7-3: 70697248A/G
7-3: 70724786A/G
7-3: 70774924A/G
7-3: 70866808C/A
7-3: 70914377G/A
7-3: 70976387A/G
7-3: 70994071C/T
7-3: 71141369G/T
7-3: 71160643C/A
7-3: 71188664A/G
7-3: 71287516G/A
7-3: 71334699C/A
7-3: 71349738G/A
7-3: 71421229T/C
7-3: 71441792C/A
7-3: 71475045A/T
7-3: 71475340G/T
7-3: 71476503A/C
7-3: 71476529C/A
7-3: 71476880G/A
7-3: 71477864G/A
7-3: 71490608G/A
7-3: 71492266T/A
7-3: 71522572G/A
7-3: 71554301A/G
7-3: 71603204G/A
7-3: 71655883A/G
7-3: 71739932G/T
7-3: 71804152G/A
7-3: 71816392G/A
7-3: 71878914T/A
7-3: 71925157T/C
7-3: 71990654A/G
7-3: 72017529C/T
7-3: 72051853A/G
7-3: 72174791C/T
7-3: 72246444C/A
7-3: 72393167A/G
7-3: 72554810T/C
7-3: 72608144G/A
7-3: 72695809T/C
7-3: 72706860G/T
7-3: 72737336G/A
7-3: 72765053A/G
7-3: 72831507T/A
7-3: 72877492C/A
7-3: 72986656A/G
7-3: 73070800G/T
7-3: 73108132C/T
7-3: 73186507C/T
7-3: 73197367G/A
7-3: 73208237A/T
7-3: 73234333G/A
7-3: 73761950G/A
7-3: 73978644A/C
7-3: 74042381T/C
7-3: 74073332A/C
7-3: 74119456T/G
7-3: 74146447G/A
7-3: 74232555G/A
7-3: 74238315T/G
7-3: 74344959A/G
7-3: 74359406C/T
7-3: 74365172G/A
7-3: 74391790G/A
7-3: 74425017A/G
7-3: 74446623G/A
7-3: 74591573C/A
7-3: 74728423G/T
7-3: 74790372C/T
7-3: 74911314A/G
7-3: 75038061C/T
7-3: 75141311A/G
7-3: 75339538T/G
7-3: 75346007G/A
7-3: 75388195A/G
7-3: 75509554C/A
7-3: 75535112A/G
7-3: 75546128G/C
7-3: 75567455C/T
7-3: 75595502T/C
7-3: 75606697G/A
7-3: 75636729T/C
7-3: 75683432C/A
7-3: 75715506C/T
7-3: 75732339G/C
7-3: 75735364G/A
7-3: 75757859T/C
7-3: 75814292A/G
7-3: 75838610A/T
7-3: 75886362G/A
7-3: 75904539A/G
7-3: 75912164A/C
7-3: 75931782T/C
7-3: 75962609T/A
7-3: 76085534A/T
7-3: 76148218A/C
7-3: 76203025A/G
7-3: 76280615C/T
7-3: 76336385C/T
7-3: 76356596C/A
7-3: 76447432A/G
7-3: 76669414G/A
7-3: 76787748C/T
7-3: 76799429G/A
7-3: 76823322C/T
7-3: 76965364A/G
7-3: 77237911T/C
7-3: 77352102G/A
7-3: 77352486G/C
7-3: 77353233G/A
7-3: 77381401A/G
7-3: 77407551G/A
7-3: 77418446T/C
7-3: 77652095C/T
7-3: 77700732C/T
7-3: 77718003T/C
7-3: 77719907T/G
7-3: 77720267G/T
7-3: 77756882C/T
7-3: 77815690A/G
7-3: 77930407T/C
7-3: 77936251C/A
7-3: 78001223A/C
7-3: 78116368T/A
7-3: 78137639G/A
7-3: 78170826C/G
7-3: 78192000G/A
7-3: 78202318T/A
7-3: 78218280G/A
7-3: 78230010G/A
7-3: 78230184C/T
7-3: 78230683C/T
7-3: 78256427A/T
7-3: 78558837A/T
7-3: 78614933G/A
7-3: 78705541G/A
7-3: 78806372C/T
7-3: 78827329C/T
7-3: 78883331A/C
7-3: 78933887A/C
7-3: 78971677A/C
7-3: 78994808G/A
7-3: 79005300T/C
7-3: 79067822A/C
7-3: 79159138A/G
7-3: 79217563C/T
7-3: 79325468T/A
7-3: 79350587G/A
7-3: 79358794C/G
7-3: 79400218C/T
7-3: 79432719G/A
7-3: 79477547T/C
7-3: 79508889C/A
7-3: 79569864T/C
7-3: 79646402T/C
7-3: 79694836T/A
7-3: 79773091T/G
7-3: 79809730G/T
7-3: 79850098T/C
7-3: 80004081A/T
7-3: 80019525T/C
7-3: 80040064G/A
7-3: 80165640A/G
7-3: 80178917G/A
7-3: 80255602G/T
7-3: 80298946T/C
7-3: 80369046C/T
7-3: 80496624T/A
7-3: 80680916G/A
7-3: 80683552C/T
7-3: 80696473A/C
7-3: 80729391C/T
7-3: 80730197T/G
7-3: 80784822C/T
7-3: 80855173G/T
7-3: 80955599A/C
7-3: 81092271C/T
7-3: 81248696T/C
7-3: 81357285A/T
7-3: 81367548A/G
7-3: 81391393A/G
7-3: 81452916G/A
7-3: 81462864G/A
7-3: 81472252C/T
7-3: 81489017G/C
7-3: 81546808G/A
7-3: 81572911T/C
7-3: 81784164G/T
7-3: 81790873G/C
7-3: 81794430A/C
7-3: 81896266A/C
7-3: 81996370C/T
7-3: 82000575A/G
7-3: 82007166C/A
7-3: 82019510G/T
7-3: 82035058A/G
7-3: 82046857C/T
7-3: 82058851C/T
7-3: 82074779T/C
7-3: 82075121T/C
7-3: 82076414T/C
7-3: 82135570G/A
7-3: 82251215A/G
7-3: 82256128C/T
7-3: 82271654T/C
7-3: 82290015T/A
7-3: 82296599T/A
7-3: 82296854C/T
7-3: 82296976G/T
7-3: 82417576C/A
7-3: 82422411A/G
7-3: 82433620G/C
7-3: 82436640A/G
7-3: 82545272T/C
7-3: 82556694T/G
7-3: 82689445A/G
7-3: 82700153A/G
7-3: 82721758T/G
7-3: 82810210C/T
7-3: 82841504T/G
7-3: 82853334T/A
7-3: 82904702G/A
7-3: 82929995C/A
7-3: 82992573A/C
7-3: 83048409A/T
7-3: 83074831A/G
7-3: 83111255G/A
7-3: 83121797G/A
7-3: 83207895A/G
7-3: 83423573G/T
7-3: 83516931G/A
7-3: 83646649G/A
7-3: 83731110A/T
7-3: 83839337C/A
7-3: 83882610T/A
7-3: 83900770G/T
7-3: 83904852A/T
7-3: 83915655T/C
7-3: 83918022C/T
7-3: 83918070C/T
7-3: 83918152A/G
7-3: 83918375C/T
7-3: 83918526C/T
7-3: 83918749C/T
7-3: 83918887G/A
7-3: 83919105G/A
7-3: 83919228G/A
7-3: 83919451G/A
7-3: 83919707C/T
7-3: 83941117C/T
7-3: 83941663C/T
7-3: 83942789C/T
7-3: 83949409G/A
7-3: 83961451T/A
7-3: 83997221C/G
7-3: 84058615C/T
7-3: 84099423A/T
7-3: 84133787A/C
7-3: 84162532T/C
7-3: 84214019C/T
7-3: 84238994T/G
7-3: 84332253A/T
7-3: 84336087C/T
7-3: 84358886C/T
7-3: 84443784C/T
7-3: 84530091T/C
7-3: 84687427T/A
7-3: 84886620T/C
7-3: 84962993G/A
7-3: 85063996C/T
7-3: 85161880A/T
7-3: 85241892G/A
7-3: 85448343T/A
7-3: 85465810C/G
7-3: 85672074A/C
7-3: 85681626G/A
7-3: 85774946A/G
7-3: 85956056C/T
7-3: 86051556C/G
7-3: 86119207C/T
7-3: 86131116C/T
7-3: 86302723G/T
7-3: 86325450T/G
7-3: 86349551T/C
7-3: 86484922A/T
7-3: 86567595T/A
7-3: 86650556A/G
7-3: 86825413G/A
7-3: 86900981C/T
7-3: 87070237G/A
7-3: 87115107A/T
7-3: 87286426C/T
7-3: 87442019T/C
7-3: 87479233G/T
7-4: 238800G/T
7-4: 410597G/C
7-4: 661843A/T
7-4: 683841T/A
7-4: 701172C/T
7-4: 810262A/G
7-4: 859825T/C
7-4: 970678T/A
7-4: 1002776T/C
7-4: 1040463T/C
7-4: 1132106G/A
7-4: 1178526T/C
7-4: 1190365G/A
7-4: 1257828A/G
7-4: 1300973G/A
7-4: 1303717C/A
7-4: 1314295C/T
7-4: 1324821G/A
7-4: 1334995G/A
7-4: 1364056A/G
7-4: 1395771A/G
7-4: 1645443A/C
7-4: 1671999T/A
7-4: 1739490A/T
7-4: 1765645T/C
7-4: 1827978T/G
7-4: 1853264T/C
7-4: 1878300T/A
7-4: 1902870T/A
7-4: 1911215C/T
7-4: 1911550A/G
7-4: 1911614G/A
7-4: 1911735A/G
7-4: 1911805C/T
7-4: 1912139C/T
7-4: 1912209T/A
7-4: 1912368T/C
7-4: 1912484C/A
7-4: 1912921A/G
7-4: 1928496A/G
7-4: 1946589C/T
7-4: 1981667T/C
7-4: 2029947A/C
7-4: 2030066G/A
7-4: 2030195G/A
7-4: 2047642A/T
7-4: 2093050G/A
7-4: 2093584T/A
7-4: 2118195A/T
7-4: 2138974G/A
7-4: 2158522G/A
7-4: 2187271G/A
7-4: 2187447G/T
7-4: 2226275T/C
7-4: 2293018G/A
7-4: 2343470C/T
7-4: 2396183A/C
7-4: 2428633A/G
7-4: 2536089A/C
7-4: 2557985T/C
7-4: 2590010C/T
7-4: 2717388G/T
7-4: 2783664C/T
7-4: 2824474A/G
7-4: 2837551C/T
7-4: 2857600G/A
7-4: 2873464T/C
7-4: 2900541A/G
7-4: 2902446G/C
7-4: 2914362A/G
7-4: 2950010T/C
7-4: 2974282G/A
7-4: 3054860T/G
7-4: 3140019A/G
7-4: 3162365G/A
7-4: 3175860G/C
7-4: 3185025C/T
7-4: 3213244C/T
7-4: 3306550C/T
7-4: 3309617G/A
7-4: 3309879T/C
7-4: 3317605T/A
7-4: 3317807T/A
7-4: 3327323A/G
7-4: 3328792T/G
7-4: 3334030A/G
7-4: 3334327C/A
7-4: 3337619T/A
7-4: 3337792A/G
7-4: 3338114T/G
7-4: 3342995C/A
7-4: 3354173A/T
7-4: 3355076C/T
7-4: 3355155G/A
7-4: 3355713G/A
7-4: 3413295C/G
7-4: 3448122A/G
7-4: 3502540C/T
7-4: 3502601T/C
7-4: 3502732G/A
7-4: 3503141T/C
7-4: 3513991C/A
7-4: 3524976A/T
7-4: 3572408T/A
7-4: 3633658C/T
7-4: 3669622A/T
7-4: 3725863T/C
7-4: 3744619T/C
7-4: 3759675T/C
7-4: 3771250A/C
7-4: 3786882G/A
7-4: 3844650T/C
7-4: 3887750T/C
7-4: 3901192G/A
7-4: 3929550G/A
7-4: 4128497T/C
7-4: 4154898G/A
7-4: 4203061G/C
7-4: 4265950C/T
7-4: 4320253A/C
7-4: 4384525A/G
7-4: 4445345G/A
7-4: 4472806G/A
7-4: 4500112T/A
7-4: 4516219T/G
7-4: 4519720T/C
7-4: 4583332A/C
7-4: 4606471G/T
7-4: 4606537A/G
7-4: 4644112A/C
7-4: 4673221A/G
7-4: 4679575C/T
7-4: 4783176T/C
7-4: 4878431A/G
7-4: 4885193A/G
7-4: 4901883T/C
7-4: 5028742T/C
7-4: 5080430T/A
7-4: 5112058G/A
7-4: 5197697T/C
7-4: 5211846C/T
7-4: 5229251A/C
7-4: 5229298A/C
7-4: 5229403C/T
7-4: 5229546C/T
7-4: 5229847G/A
7-4: 5230106G/A
7-4: 5230323C/T
7-4: 5239015T/A
7-4: 5239081G/T
7-4: 5239242G/A
7-4: 5239401T/C
7-4: 5239693T/C
7-4: 5239773T/A
7-4: 5239949G/A
7-4: 5240077C/T
7-4: 5240271T/A
7-4: 5240349C/A
7-4: 5240822G/A
7-4: 5240923C/T
7-4: 5241106C/T
7-4: 5241111A/T
7-4: 5241315A/G
7-4: 5241411C/A
7-4: 5259283G/T
7-4: 5285222C/T
7-4: 5379298C/T
7-4: 5517350A/T
7-4: 5607535G/A
7-4: 5626006T/A
7-4: 5649734A/G
7-4: 5689180G/A
7-4: 5719237A/G
7-4: 5783858A/T
7-4: 5829779C/T
7-4: 5897597C/A
7-4: 5929346T/A
7-4: 5958519C/T
7-4: 5959330G/A
7-4: 5980125T/C
7-4: 5993454G/A
7-4: 6003737T/A
7-4: 6003759T/C
7-4: 6080870G/A
7-4: 6148012G/A
7-4: 6217606T/C
7-4: 6244891A/G
7-4: 6296070T/A
7-4: 6309223C/T
7-4: 6413116C/T
7-4: 6440782T/C
7-4: 6465581T/C
7-4: 6467749C/T
7-4: 6478760G/T
7-4: 6479209T/C
7-4: 6522746T/G
7-4: 6522762A/T
7-4: 6547823T/A
7-4: 6612625A/C
7-4: 6646875C/T
7-4: 6678290T/C
7-4: 6690402C/T
7-4: 6719902G/A
7-4: 6753364C/T
7-4: 6758094C/T
7-4: 6775781T/C
7-4: 6807813C/G
7-4: 6916233A/T
7-4: 6983835T/A
7-4: 6998188G/T
7-4: 6998192G/A
7-4: 6998327T/C
7-4: 6998706C/T
7-4: 6998709C/G
7-4: 6998817T/C
7-4: 6998947C/T
7-4: 7009224G/A
7-4: 7026550T/C
7-4: 7097159A/C
7-4: 7166590C/G
7-4: 7193632G/C
7-4: 7222347C/A
7-4: 7222746A/G
7-4: 7270472A/T
7-4: 7286720C/T
7-4: 7297115T/G
7-4: 7306126G/A
7-4: 7311787G/A
7-4: 7341685T/C
7-4: 7382522T/A
7-4: 7438701T/C
7-4: 7441379A/C
7-4: 7453756A/G
7-4: 7486271A/T
7-4: 7577978A/G
7-4: 7604663C/G
7-4: 7613370A/G
7-4: 7623379G/C
7-4: 7688159A/T
7-4: 7704558C/T
7-4: 7705161T/A
7-4: 7716094C/T
7-4: 7716675T/A
7-4: 7716785A/C
7-4: 7717515T/C
7-4: 7717516G/C
7-4: 7741769C/T
7-4: 7751488A/G
7-4: 7776272T/G
7-4: 7790726T/C
7-4: 7832381G/A
7-4: 7919273C/T
7-4: 8010512C/T
7-4: 8013872C/T
7-4: 8041529C/G
7-4: 8073767G/A
7-4: 8087942C/T
7-4: 8094863G/T
7-4: 8098219C/T
7-4: 8131664A/G
7-4: 8191281C/T
7-4: 8229982G/T
7-4: 8333196C/T
7-4: 8362414A/G
7-4: 8376500G/A
7-4: 8392943T/C
7-4: 8409588C/T
7-4: 8432107A/T
7-4: 8432143G/C
7-4: 8432245G/A
7-4: 8433213A/G
7-4: 8433482C/T
7-4: 8433586A/G
7-4: 8433788G/A
7-4: 8433801T/C
7-4: 8442347T/A
7-4: 8483770G/A
7-4: 8500171T/A
7-4: 8549531G/C
7-4: 8615936A/C
7-4: 8675074C/T
7-4: 8716864A/T
7-4: 8761405A/G
7-4: 8810315G/A
7-4: 8912975T/C
7-4: 8915957A/C
7-4: 8979891A/G
7-4: 9022289A/G
7-4: 9055929G/A
7-4: 9063108C/T
7-4: 9087487A/G
7-4: 9164222A/G
7-4: 9209781A/G
7-4: 9252732C/A
7-4: 9256125G/A
7-4: 9335462C/T
7-4: 9447353T/C
7-4: 9525363G/A
7-4: 9553750C/A
7-4: 9633569G/T
7-4: 9635153G/A
7-4: 9644212C/A
7-4: 9666961A/G
7-4: 9781580T/A
7-4: 9880140G/A
7-4: 9906722A/G
7-4: 9930978A/T
7-4: 9983768G/T
7-4: 10131263C/T
7-4: 10161792G/A
7-4: 10229791G/A
7-4: 10258807A/T
7-4: 10295069G/T
7-4: 10354598A/C
7-4: 10392934A/T
7-4: 10425551C/T
7-4: 10428827C/T
7-4: 10454689A/G
7-4: 10481092T/A
7-4: 10513043C/A
7-4: 10534201C/G
7-4: 10553876T/C
7-4: 10553916A/G
7-4: 10556707C/T
7-4: 10567664C/T
7-4: 10587871A/T
7-4: 10631619A/T
7-4: 10716491C/G
7-4: 10751567G/A
7-4: 10883946G/T
7-4: 11029041T/C
7-4: 11056588T/G
7-4: 11056652A/C
7-4: 11057252T/C
7-4: 11057563T/A
7-4: 11057695A/G
7-4: 11066952C/T
7-4: 11093156A/G
7-4: 11094263C/T
7-4: 11094304T/A
7-4: 11096242A/G
7-4: 11096453T/A
7-4: 11096555G/T
7-4: 11097822A/C
7-4: 11097901C/G
7-4: 11097991T/A
7-4: 11098326C/T
7-4: 11098352A/T
7-4: 11098429G/A
7-4: 11098552A/C
7-4: 11098642G/T
7-4: 11098659C/T
7-4: 11098796C/T
7-4: 11098862A/G
7-4: 11105188G/A
7-4: 11168380G/A
7-4: 11170036G/A
7-4: 11170114G/A
7-4: 11170360A/G
7-4: 11170543G/A
7-4: 11170782T/C
7-4: 11170973A/G
7-4: 11171045T/C
7-4: 11171081G/A
7-4: 11171393G/A
7-4: 11171494A/G
7-4: 11171546C/A
7-4: 11171641G/A
7-4: 11171771C/T
7-4: 11172119G/A
7-4: 11172380A/T
7-4: 11173605C/A
7-4: 11173954T/C
7-4: 11174163T/C
7-4: 11197440A/G
7-4: 11243362C/A
7-4: 11305348T/A
7-4: 11439448C/A
7-4: 11446595C/T
7-4: 11508337G/A
7-4: 11544273G/C
7-4: 11577260G/A
7-4: 11617767A/G
7-4: 11658960A/G
7-4: 11800096C/T
7-4: 11879703A/T
7-4: 11922700C/T
7-4: 11958177G/A
7-4: 12033620T/C
7-4: 12084675A/G
7-4: 12096748T/C
7-4: 12115562A/T
7-4: 12125802A/G
7-4: 12164500T/C
7-4: 12196324C/A
7-4: 12211852G/A
7-4: 12237566T/C
7-4: 12291291T/G
7-4: 12316946C/T
7-4: 12367180T/A
7-4: 12386152T/A
7-4: 12446131A/C
7-4: 12447527C/T
7-4: 12489116C/A
7-4: 12511518G/A
7-4: 12533604G/T
7-4: 12545015T/A
7-4: 12564934G/A
7-4: 12575602C/T
7-4: 12575603C/T
7-4: 12575765C/T
7-4: 12585813T/C
7-4: 12634298A/G
7-4: 12674167G/C
7-4: 12714630C/T
7-4: 12760460A/T
7-4: 12795008T/A
7-4: 12941282C/T
7-4: 12957948G/A
7-4: 13065284C/T
7-4: 13103917A/T
7-4: 13199663G/A
7-4: 13380645G/A
7-4: 13389368T/G
7-4: 13408631G/A
7-4: 13484001G/A
7-4: 13520906G/T
7-4: 13520909C/T
7-4: 13529437T/G
7-4: 13529509G/A
7-4: 13607107C/A
7-4: 13630411C/A
7-4: 13762332G/T
7-4: 13796860G/C
7-4: 13807414T/G
7-4: 13826281A/T
7-4: 13835264T/C
7-4: 13836743A/G
7-4: 13837043C/G
7-4: 13841762T/C
7-4: 14130058A/T
7-4: 14175549T/C
7-4: 14285722A/G
7-4: 14331780C/T
7-4: 14342926A/T
7-4: 14366728T/A
7-4: 14402997G/A
7-4: 14566189A/T
7-4: 14599133G/A
7-4: 14631240C/A
7-4: 14631659C/T
7-4: 14632113G/A
7-4: 14652261G/T
7-4: 14653505A/G
7-4: 14653615T/A
7-4: 14656090A/G
7-4: 14663014G/A
7-4: 14663137A/C
7-4: 14663359A/G
7-4: 14663525C/T
7-4: 14674794C/T
7-4: 14725855T/C
7-4: 14741762G/A
7-4: 14742054T/A
7-4: 14745759C/T
7-4: 14974051C/T
7-4: 15067006A/G
7-4: 15068318C/T
7-4: 15078488T/C
7-4: 15105007T/G
7-4: 15135272C/G
7-4: 15155900C/T
7-4: 15172363C/T
7-4: 15173180A/.
7-4: 15212915G/T
7-4: 15247729A/T
7-4: 15247913T/A
7-4: 15265958G/C
7-4: 15270531G/A
7-4: 15270749G/A
7-4: 15270926A/T
7-4: 15271031C/G
7-4: 15271034T/C
7-4: 15272354T/C
7-4: 15272457G/A
7-4: 15294714A/T
7-4: 15309067C/T
7-4: 15334886G/A
7-4: 15439186C/T
7-4: 15505176T/C
7-4: 15529947G/T
7-4: 15773583G/A
7-4: 15941904G/A
7-4: 15941926C/T
7-4: 15941928C/A
7-4: 15941971A/G
7-4: 15941983A/T
7-4: 15942012A/T
7-4: 15942044A/G
7-4: 15942052C/T
7-4: 16165818A/C
7-4: 16183564A/G
7-4: 16185026T/A
7-4: 16186675C/T
7-4: 16187464C/A
7-4: 16406547G/C
7-4: 16425719C/T
7-4: 16440992A/G
7-4: 16510601T/C
7-4: 16525504G/A
7-4: 16579469G/T
7-4: 16679586G/A
7-4: 16708550T/C
7-4: 16747284G/A
7-4: 16791257A/T
7-4: 16791267T/C
7-4: 16836903T/G
7-4: 16865615T/C
7-4: 16901239G/A
7-4: 16920003T/C
7-4: 16933246T/G
7-4: 16943636A/G
7-4: 16963911C/A
7-4: 17045186T/A
7-4: 17093801C/A
7-4: 17124629T/C
7-4: 17138398A/T
7-4: 17138419C/T
7-4: 17149441A/G
7-4: 17161289T/A
7-4: 17165768T/.
7-4: 17172985G/A
7-4: 17192771T/G
7-4: 17204736T/A
7-4: 17266663C/G
7-4: 17272523T/A
7-4: 17277250A/G
7-4: 17315776A/G
7-4: 17335273T/C
7-4: 17370601A/T
7-4: 17397562T/C
7-4: 17420766G/T
7-4: 17499694G/A
7-4: 17504901G/T
7-4: 17505711C/T
7-4: 17541025A/T
7-4: 17597796A/C
7-4: 17729430C/T
7-4: 17731505A/G
7-4: 17768732C/A
7-4: 17801016A/G
7-4: 17814231G/T
7-4: 17848695C/T
7-4: 17888762T/A
7-4: 18125311T/G
7-4: 18128304A/G
7-4: 18287530C/A
7-4: 18693345G/A
7-4: 18759035T/A
7-4: 18776579A/G
7-4: 18845630T/C
7-4: 19065301C/T
7-4: 19151880G/A
7-4: 19151984T/C
7-4: 19151991A/T
7-4: 19223429G/A
7-4: 19464818C/A
7-4: 19649306G/T
7-4: 19671277T/C
7-4: 19687258T/C
7-4: 19816248T/G
7-4: 19822468G/A
7-4: 19862844C/T
7-4: 20019463A/T
7-4: 20030505G/T
7-4: 20073509T/C
7-4: 20212084T/A
7-4: 20267060T/C
7-4: 20307677A/T
7-4: 20390373G/A
7-4: 20403662C/A
7-4: 20403902T/A
7-4: 20404016T/C
7-4: 20404563G/A
7-4: 20405635A/G
7-4: 20406219G/T
7-4: 20408818T/C
7-4: 20424019C/T
7-4: 20463798A/G
7-4: 20476234A/T
7-4: 20492469C/T
7-4: 20718453G/A
7-4: 20806308G/A
7-4: 20946594T/C
7-4: 20968503A/G
7-4: 21065976C/T
7-4: 21314228G/A
7-4: 21325628C/A
7-4: 21327545C/T
7-4: 21378278G/T
7-4: 21406190C/T
7-4: 21439050T/C
7-4: 21469292A/G
7-4: 21503738T/C
7-4: 21557429T/C
7-4: 21596268G/T
7-4: 21609485T/G
7-4: 21609740T/A
7-4: 21661714C/T
7-4: 21764180G/T
7-4: 21939852C/A
7-4: 22021444T/A
7-4: 22154589C/G
7-4: 22204756T/A
7-4: 22250136T/C
7-4: 22268239A/G
7-4: 22280797A/C
7-4: 22304037G/C
7-4: 22350718A/G
7-4: 22397183C/G
7-4: 22455927C/T
7-4: 22526668C/T
7-4: 22551539A/G
7-4: 22641027C/G
7-4: 22656753T/G
7-4: 22695539C/A
7-4: 22723123C/T
7-4: 22877009G/C
7-4: 23109342G/C
7-4: 23233542C/G
7-4: 23261001T/C
7-4: 23272939A/T
7-4: 23272951C/A
7-4: 23273056A/G
7-4: 23278956T/C
7-4: 23279760T/C
7-4: 23333753A/G
7-4: 23405419C/G
7-4: 23405525A/G
7-4: 23406214C/T
7-4: 23410619C/A
7-4: 23410837G/A
7-4: 23410845A/G
7-4: 23410878G/T
7-4: 23410901T/A
7-4: 23410930T/A
7-4: 23410932C/T
7-4: 23411584C/T
7-4: 23411585T/.
7-4: 23411598A/T
7-4: 23411599G/T
7-4: 23411602C/.
7-4: 23411605A/.
7-4: 23467132A/G
7-4: 23511749G/A
7-4: 23542332T/C
7-4: 23568445G/C
7-4: 23603967G/A
7-4: 23652319C/T
7-4: 23663159C/A
7-4: 23673439T/G
7-4: 23691348T/A
7-4: 23708171A/T
7-4: 23725879T/A
7-4: 23751983A/G
7-4: 23780531C/T
7-4: 23799099C/T
7-4: 23809669G/A
7-4: 23810946A/G
7-4: 23945045T/G
7-4: 23997085A/G
7-4: 24031757C/T
7-4: 24130492C/A
7-4: 24155169A/G
7-4: 24223553C/A
7-4: 24259293C/T
7-4: 24313102G/T
7-4: 24416947C/T
7-4: 24465608G/A
7-4: 24561291G/A
7-4: 24569687C/G
7-4: 24588520T/A
7-4: 24632583G/A
7-4: 24633567G/T
7-4: 24644425G/A
7-4: 24724741A/G
7-4: 24724912T/A
7-4: 24752942C/T
7-4: 24762508G/A
7-4: 24762864G/T
7-4: 24763208C/T
7-4: 24772218C/T
7-4: 24794896A/C
7-4: 24799703A/T
7-4: 24799838T/C
7-4: 24799997G/T
7-4: 24802593C/T
7-4: 24803341G/T
7-4: 24803554A/G
7-4: 24803685T/A
7-4: 24803838T/A
7-4: 24847149C/T
7-4: 24882195G/A
7-4: 24882196G/T
7-4: 24915753G/A
7-4: 24933539A/G
7-4: 24962829T/A
7-4: 24963006A/T
7-4: 24963109C/A
7-4: 24963216C/T
7-4: 24966782T/C
7-4: 24976614C/G
7-4: 24978944G/C
7-4: 24978963C/G
7-4: 25031058T/G
7-4: 25037386G/A
7-4: 25050151A/G
7-4: 25060494C/A
7-4: 25098644C/T
7-4: 25129467C/T
7-4: 25137618G/A
7-4: 25177535C/T
7-4: 25199634A/T
7-4: 25216972T/A
7-4: 25231399G/T
7-4: 25241614G/A
7-4: 25252066G/A
7-4: 25262696A/G
7-4: 25270377G/T
7-4: 25270420C/A
7-4: 25275368A/G
7-4: 25276117A/G
7-4: 25276201A/G
7-4: 25276218G/C
7-4: 25276477C/T
7-4: 25276686A/G
7-4: 25282720C/A
7-4: 25296930G/A
7-4: 25312198G/C
7-4: 25325099T/A
7-4: 25340316G/C
7-4: 25340441C/T
7-4: 25348087C/A
7-4: 25348355A/T
7-4: 25348409G/A
7-4: 25348472C/T
7-4: 25349015C/A
7-4: 25349059C/T
7-4: 25416423C/T
7-4: 25433251C/A
7-4: 25444438G/A
7-4: 25457929T/C
7-4: 25482937G/A
7-4: 25495954C/T
7-4: 25535090C/T
7-4: 25546881C/T
7-4: 25551827A/G
7-4: 25557477C/T
7-4: 25575970C/T
7-4: 25586107C/T
7-4: 25605166C/T
7-4: 25623367A/C
7-4: 25630711C/T
7-4: 25630974G/T
7-4: 25634214G/A
7-4: 25636410G/A
7-4: 25662936C/T
7-4: 25677792T/A
7-4: 25678833T/C
7-4: 25679006C/G
7-4: 25689715C/T
7-4: 25689904T/G
7-4: 25690163G/A
7-4: 25690410A/G
7-4: 25690442G/C
7-4: 25694720C/T
7-4: 25714103T/C
7-4: 25741225A/G
7-4: 25752232G/A
7-4: 25754167A/G
7-4: 25754336T/G
7-4: 25755727C/G
7-4: 25762948A/C
7-4: 25771114C/G
7-4: 25774001A/G
7-4: 25861545G/A
7-4: 25891260C/T
7-4: 25968695A/C
7-4: 26029069A/T
7-4: 26059591A/G
7-4: 26146346T/C
7-4: 26195742A/T
7-4: 26205540T/C
7-4: 26265848C/G
7-4: 26276519C/T
7-4: 26322730C/G
7-4: 26333961T/G
7-4: 26336953T/A
7-4: 26337955C/T
7-4: 26338071C/T
7-4: 26353794G/A
7-4: 26401677T/C
7-4: 26440322G/C
7-4: 26529147G/C
7-4: 26539611C/T
7-4: 26546759C/T
7-4: 26546915C/T
7-4: 26546989A/G
7-4: 26547122A/T
7-4: 26547228C/A
7-4: 26548554A/G
7-4: 26549184C/A
7-4: 26550228T/A
7-4: 26561317G/A
7-4: 26591953G/A
7-4: 26612236C/G
7-4: 26612649A/T
7-4: 26644278A/G
7-4: 26699610T/C
7-4: 26734897G/T
7-4: 26746504A/T
7-4: 26765224A/C
7-4: 26771439G/A
7-4: 26778096G/A
7-4: 26778672G/A
7-4: 26779105G/A
7-4: 26848723G/A
7-4: 26869129C/G
7-4: 26891138C/A
7-4: 26902976T/G
7-4: 26933361G/A
7-4: 26960457C/T
7-4: 26979572C/T
7-4: 26980144C/T
7-4: 26991403C/T
7-4: 26991518A/G
7-4: 26998896G/C
7-4: 27020377T/A
7-4: 27167988C/T
7-4: 27321088G/A
7-4: 27341483C/A
7-4: 27368469T/C
7-4: 27413499T/A
7-4: 27429221T/A
7-4: 27443624G/C
7-4: 27482840G/T
7-4: 27515513T/G
7-4: 27516900T/C
7-4: 27536162T/G
7-4: 27559989T/C
7-4: 27617833T/A
7-4: 27655813A/G
7-4: 27671051A/G
7-4: 27691344A/G
7-4: 27727473T/C
7-4: 27749274G/A
7-4: 27762073C/T
7-4: 27762280A/T
7-4: 27762444T/A
7-4: 27762641G/A
7-4: 27762757G/A
7-4: 27763046C/T
7-4: 27772294A/T
7-4: 27966848G/T
7-4: 28009187A/T
7-4: 28022355T/G
7-4: 28029221G/A
7-4: 28040402A/G
7-4: 28066960A/G
7-4: 28099239C/T
7-4: 28131862C/T
7-4: 28160007G/A
7-4: 28194159G/A
7-4: 28240831T/A
7-4: 28261530A/T
7-4: 28308514G/A
7-4: 28410674A/T
7-4: 28467361C/T
7-4: 28503426A/T
7-4: 28517448G/T
7-4: 28537246T/C
7-4: 28559268G/A
7-4: 28561758G/A
7-4: 28561859A/G
7-4: 28561946T/C
7-4: 28561977C/A
7-4: 28562108C/A
7-4: 28574587A/C
7-4: 28643140A/G
7-4: 28655170T/C
7-4: 28850690C/T
7-4: 28851806C/T
7-4: 28880523A/G
7-4: 28908413T/C
7-4: 28949971C/A
7-4: 28980678A/T
7-4: 29010456T/C
7-4: 29115346A/G
7-4: 29119461C/T
7-4: 29322843G/A
7-4: 29323215G/A
7-4: 29349590G/T
7-4: 29366094G/T
7-4: 29390189G/A
7-4: 29442321T/C
7-4: 29474568C/T
7-4: 29564976G/A
7-4: 29578575A/C
7-4: 29657439T/A
7-4: 29729447T/A
7-4: 29744291G/C
7-4: 29744633T/A
7-4: 29813798C/T
7-4: 29828716A/T
7-4: 29839051A/T
7-4: 29864207T/G
7-4: 29890951T/G
7-4: 29891514T/C
7-4: 29953137T/C
7-4: 30002983G/A
7-4: 30030145G/A
7-4: 30068755G/A
7-4: 30103062T/C
7-4: 30144466C/A
7-4: 30259480T/C
7-4: 30327504C/T
7-4: 30369833A/G
7-4: 30443648C/T
7-4: 30459463A/G
7-4: 30525097T/C
7-4: 30546697C/G
7-4: 30559654G/A
7-4: 30571728C/G
7-4: 30580758C/T
7-4: 30582765T/C
7-4: 30586209A/G
7-4: 30606835A/C
7-4: 30625138G/T
7-4: 30653615C/T
7-4: 30654564T/C
7-4: 30654739A/G
7-4: 30660521C/T
7-4: 30660710A/C
7-4: 30766908C/T
7-4: 30785927A/T
7-4: 30803995A/G
7-4: 30849428A/G
7-4: 30873376G/A
7-4: 30874947A/G
7-4: 30884288C/T
7-4: 30908589G/A
7-4: 30929804C/T
7-4: 30943259T/G
7-4: 31003449T/C
7-4: 31006653G/A
7-4: 31014655G/A
7-4: 31054888C/T
7-4: 31056915A/G
7-4: 31065268C/T
7-4: 31067192T/G
7-4: 31080671T/A
7-4: 31090734G/T
7-4: 31143920A/T
7-4: 31156529T/G
7-4: 31169348C/T
7-4: 31174062A/G
7-4: 31181921C/A
7-4: 31187063T/G
7-4: 31189221T/A
7-4: 31189253A/G
7-4: 31189334C/T
7-4: 31190828T/C
7-4: 31190863T/C
7-4: 31190868T/C
7-4: 31194500A/T
7-4: 31210529A/T
7-4: 31263931C/A
7-4: 31322040G/A
7-4: 31348758G/C
7-4: 31375626G/A
7-4: 31412818G/T
7-4: 31455327A/T
7-4: 31505399T/A
7-4: 31524463A/G
7-4: 31568497T/A
7-4: 31772202C/T
7-4: 31808153A/T
7-4: 31827554A/T
7-4: 31833464G/A
7-4: 31899782G/A
7-4: 31910937A/T
7-4: 31929249A/G
7-4: 31939612C/A
7-4: 31946385A/G
7-4: 31949725C/T
7-4: 32055802G/A
7-4: 32186034C/T
7-4: 32197272A/T
7-4: 32214897T/C
7-4: 32259187A/G
7-4: 32295732G/A
7-4: 32328874G/T
7-4: 32355384G/T
7-4: 32361708C/T
7-4: 32361754C/A
7-4: 32361755A/.
7-4: 32397958C/T
7-4: 32434891C/T
7-4: 32472762A/G
7-4: 32490434G/A
7-4: 32522892T/C
7-4: 32642776A/G
7-4: 32672707C/T
7-4: 32685673A/T
7-4: 32718947G/C
7-4: 32752533T/C
7-4: 32784426C/T
7-4: 32861986T/C
7-4: 32871214C/T
7-4: 32948702C/T
7-4: 32993222A/G
7-4: 33005489G/T
7-4: 33023929A/T
7-4: 33088960C/A
7-4: 33112998A/C
7-4: 33131485A/G
7-4: 33195056A/G
7-4: 33216152A/G
7-4: 33218183G/T
7-4: 33236742T/A
7-4: 33264830G/A
7-4: 33376096A/G
7-4: 33395366T/A
7-4: 33409928G/C
7-4: 33435913A/T
7-4: 33446182G/A
7-4: 33472417T/C
7-4: 33504864A/G
7-4: 33507433G/T
7-4: 33507575T/C
7-4: 33516120T/G
7-4: 33587118C/G
7-4: 33597591A/T
7-4: 33598503A/C
7-4: 33598564T/C
7-4: 33602090C/A
7-4: 33643432G/A
7-4: 33669631C/T
7-4: 33725828G/A
7-4: 33809189A/G
7-4: 33819382C/T
7-4: 33855114C/T
7-4: 33891491C/G
7-4: 33905357G/T
7-4: 33914467A/T
7-4: 33939697C/T
7-4: 33959135C/T
7-4: 34090214G/A
7-4: 34150108G/A
7-4: 34204159T/C
7-4: 34243074G/A
7-4: 34321820A/G
7-4: 34348646G/C
7-4: 34360508T/A
7-4: 34389506C/A
7-4: 34419096G/A
7-4: 34426693G/A
7-4: 34432410T/G
7-4: 34447338T/C
7-4: 34455667C/G
7-4: 34455872T/G
7-4: 34474452G/A
7-4: 34485566A/T
7-4: 34497527A/T
7-4: 34498176T/A
7-4: 34499224A/C
7-4: 34588381G/A
7-4: 34603006T/C
7-4: 34669674T/G
7-4: 34681933T/C
7-4: 34744277C/T
7-4: 34756726T/G
7-4: 34773626T/A
7-4: 34775467T/C
7-4: 34822440A/T
7-4: 34847210A/G
7-4: 34881561C/T
7-4: 34949296C/T
7-4: 34986053C/A
7-4: 35002003C/T
7-4: 35002367G/A
7-4: 35003557C/T
7-4: 35049530A/C
7-4: 35090354A/T
7-4: 35118353T/A
7-4: 35136670A/C
7-4: 35152548A/T
7-4: 35210147A/T
7-4: 35256802C/T
7-4: 35324192A/C
7-4: 35337774G/A
7-4: 35342184C/T
7-4: 35470951A/T
7-4: 35501455T/A
7-4: 35502459A/C
7-4: 35502635T/A
7-4: 35503085A/C
7-4: 35503222C/T
7-4: 35503230C/T
7-4: 35503384A/G
7-4: 35503513G/A
7-4: 35503662G/A
7-4: 35511736A/T
7-4: 35527316A/G
7-4: 35668102A/C
7-4: 35691061G/A
7-4: 35701470G/A
7-4: 35717104C/T
7-4: 35730209C/T
7-4: 35730599A/C
7-4: 35737719C/T
7-4: 35737852A/G
7-4: 35738309A/T
7-4: 35738477C/T
7-4: 35738729A/T
7-4: 35738851C/G
7-4: 35835148A/G
7-4: 35864757T/C
7-4: 35955109T/G
7-4: 36076916A/G
7-4: 36080161A/T
7-4: 36101612C/T
7-4: 36101702A/G
7-4: 36165777A/G
7-4: 36178347T/A
7-4: 36178574G/A
7-4: 36178727G/A
7-4: 36178838T/G
7-4: 36178945G/A
7-4: 36179107C/T
7-4: 36179231G/A
7-4: 36179337T/A
7-4: 36186205T/A
7-4: 36189268C/A
7-4: 36193667C/A
7-4: 36194890A/G
7-4: 36195061A/G
7-4: 36195063A/T
7-4: 36195377G/A
7-4: 36195688C/A
7-4: 36196598G/A
7-4: 36197490A/T
7-4: 36198226A/G
7-4: 36198229A/C
7-4: 36198231G/A
7-4: 36198353A/G
7-4: 36198465C/T
7-4: 36198576C/T
7-4: 36198688A/T
7-4: 36206052T/A
7-4: 36206210G/A
7-4: 36206458A/G
7-4: 36206562C/A
7-4: 36206684G/C
7-4: 36207015G/C
7-4: 36207129T/C
7-4: 36214677T/C
7-4: 36218417C/T
7-4: 36218765G/A
7-4: 36220849T/A
7-4: 36275891G/A
7-4: 36320477T/G
7-4: 36339618A/G
7-4: 36376338C/T
7-4: 36401836T/G
7-4: 36405607C/T
7-4: 36414424G/A
7-4: 36415657C/G
7-4: 36484189A/T
7-4: 36523238A/G
7-4: 36544747A/G
7-4: 36556854G/T
7-4: 36570939G/A
7-4: 36575369C/T
7-4: 36597113T/C
7-4: 36608720A/T
7-4: 36730396A/G
7-4: 36730566A/T
7-4: 36730856A/T
7-4: 36731074G/A
7-4: 36731955G/A
7-4: 36732762G/T
7-4: 36740695T/C
7-4: 36780079T/G
7-4: 36783235G/A
7-4: 36805243A/T
7-4: 36882667G/T
7-4: 36931913C/T
7-4: 36948799T/G
7-4: 36974466C/A
7-4: 36991060G/A
7-4: 37013464C/T
7-4: 37014402T/A
7-4: 37015183A/G
7-4: 37018886T/C
7-4: 37020436G/T
7-4: 37023501A/G
7-4: 37041225C/T
7-4: 37049744T/G
7-4: 37049846T/A
7-4: 37049966A/G
7-4: 37050068T/A
7-4: 37050277A/G
7-4: 37050278A/T
7-4: 37050411A/G
7-4: 37050643G/T
7-4: 37050685T/G
7-4: 37050880T/G
7-4: 37050885G/T
7-4: 37062343A/G
7-4: 37235259T/A
7-4: 37270171T/G
7-4: 37290875T/C
7-4: 37320214G/A
7-4: 37365373A/C
7-4: 37376490C/G
7-4: 37391265T/A
7-4: 37442325A/C
7-4: 37452992G/A
7-4: 37517462C/T
7-4: 37552014A/G
7-4: 37558885A/G
7-4: 37579123C/T
7-4: 37626774C/T
7-4: 37637763C/T
7-4: 37668817G/A
7-4: 37669769G/A
7-4: 37669775A/G
7-4: 37699995G/A
7-4: 37742016A/C
7-4: 37760806G/A
7-4: 37868456A/T
7-4: 37907787A/G
7-4: 37966561C/T
7-4: 38024204G/A
7-4: 38051214T/A
7-4: 38057964T/C
7-4: 38061399G/A
7-4: 38112949G/A
7-4: 38113505C/A
7-4: 38196279A/T
7-4: 38227397G/A
7-4: 38229760G/A
7-4: 38241803C/T
7-4: 38242987C/T
7-4: 38253094G/A
7-4: 38253147T/C
7-4: 38276861T/C
7-4: 38316510G/A
7-4: 38318470C/G
7-4: 38320244C/A
7-4: 38322992A/T
7-4: 38323840G/A
7-4: 38396269T/A
7-4: 38416817T/G
7-4: 38484648C/T
7-4: 38593227A/T
7-4: 38613736T/C
7-4: 38691192C/T
7-4: 38720691G/A
7-4: 38739864G/A
7-4: 38817063G/A
7-4: 38838288C/A
7-4: 38838482G/A
7-4: 38950004G/A
7-4: 38957484G/A
7-4: 39014253G/T
7-4: 39058480T/A
7-4: 39175756C/T
7-4: 39177412T/C
7-4: 39205802C/T
7-4: 39262558C/T
7-4: 39289697C/A
7-4: 39316777A/G
7-4: 39369131C/T
7-4: 39380591T/C
7-4: 39393548T/C
7-4: 39497993A/T
7-4: 39503690C/T
7-4: 39527563A/G
7-4: 39547238C/T
7-4: 39581696T/C
7-4: 39592292T/A
7-4: 39640815A/G
7-4: 39675387G/C
7-4: 39689017T/C
7-4: 39710566A/T
7-4: 39793713C/T
7-4: 39889582G/T
7-4: 39969381C/T
7-4: 39969887A/G
7-4: 39969924G/A
7-4: 39970018T/A
7-4: 39970395C/A
7-4: 39970543T/C
7-4: 39971607G/A
7-4: 39992233G/A
7-4: 40013813A/G
7-4: 40027488T/C
7-4: 40053805T/C
7-4: 40111174T/A
7-4: 40172224T/A
7-4: 40193052G/T
7-4: 40209009A/G
7-4: 40220089G/A
7-4: 40238816C/A
7-4: 40240467A/T
7-4: 40242468G/A
7-4: 40299921A/T
7-4: 40335935T/G
7-4: 40397017A/G
7-4: 40436333G/C
7-4: 40484259T/C
7-4: 40526690T/G
7-4: 40570406A/G
7-4: 40613558C/T
7-4: 40667369C/G
7-4: 40698396A/G
7-4: 40709485A/C
7-4: 40803484A/C
7-4: 40845806A/G
7-4: 40870669G/A
7-4: 40881890G/C
7-4: 40892699A/T
7-4: 40957744A/T
7-4: 41008028A/G
7-4: 41034706T/A
7-4: 41064784A/G
7-4: 41114598A/G
7-4: 41127533A/G
7-4: 41195087T/C
7-4: 41220078G/A
7-4: 41279267T/C
7-4: 41316726G/A
7-4: 41317302C/T
7-4: 41348316G/A
7-4: 41366202T/C
7-4: 41510236G/A
7-4: 41521377A/G
7-4: 41544918A/G
7-4: 41577925G/A
7-4: 41619214C/T
7-4: 41675235C/T
7-4: 41707573A/G
7-4: 41743777G/T
7-4: 41815545G/A
7-4: 41874832C/T
7-4: 41900021G/A
7-4: 41934293G/A
7-4: 42026151C/A
7-4: 42037476C/A
7-4: 42160564T/C
7-4: 42188101C/A
7-4: 42248434G/A
7-4: 42261176A/C
7-4: 42332975T/C
7-4: 42362553T/A
7-4: 42466854T/C
7-4: 42487622T/C
7-4: 42490995A/G
7-4: 42522738T/C
7-4: 42598094G/T
7-4: 42638185G/A
7-4: 42694855C/A
7-4: 42720404C/G
7-4: 42749343G/C
7-4: 42770381T/C
7-4: 42804579C/T
7-4: 42952104C/T
7-4: 42969643T/C
7-4: 42979772T/C
7-4: 43064278A/G
7-4: 43171526T/G
7-4: 43226123T/C
7-4: 43263496G/T
7-4: 43298867C/T
7-4: 43376057C/T
7-4: 43415618A/G
7-4: 43477123G/A
7-4: 43477485C/A
7-4: 43488010G/T
7-4: 43499070T/C
7-4: 43510013G/A
7-4: 43518354T/C
7-4: 43521073C/G
7-4: 43541273G/A
7-4: 43552781C/T
7-4: 43553169C/G
7-4: 43553624C/T
7-4: 43553871A/G
7-4: 43570509C/A
7-4: 43587273A/G
7-4: 43589596T/C
7-4: 43590259A/T
7-4: 43590380G/C
7-4: 43590593G/T
7-4: 43590601A/T
7-4: 43591004G/A
7-4: 43592395A/C
7-4: 43598603G/A
7-4: 43701056T/C
7-4: 43754903G/T
7-4: 43795054C/T
7-4: 43960230T/A
7-4: 43970233T/A
7-4: 44173643C/T
7-4: 44207208T/C
7-4: 44292479A/G
7-4: 44361433A/G
7-4: 44477782C/G
7-4: 44488081C/A
7-4: 44535668C/T
7-4: 44614972A/C
7-4: 44666133G/C
7-4: 44666429G/A
7-4: 44667578A/G
7-4: 44708805G/T
7-4: 44753410C/A
7-4: 44765241T/C
7-4: 44765521G/A
7-4: 44776105G/C
7-4: 44884025T/A
7-4: 44972036C/T
7-4: 45012198C/T
7-4: 45025289T/A
7-4: 45126948G/A
7-4: 45138136G/A
7-4: 45245580A/G
7-4: 45268046A/C
7-4: 45293191C/T
7-4: 45293527C/A
7-4: 45293669C/T
7-4: 45293699C/A
7-4: 45293702C/T
7-4: 45293930C/T
7-4: 45293988T/C
7-4: 45294178T/A
7-4: 45294206C/T
7-4: 45304274T/A
7-4: 45356390T/C
7-4: 45397855G/A
7-4: 45428887T/C
7-4: 45465280G/A
7-4: 45491019G/A
7-4: 45539870G/C
7-4: 45565938T/C
7-4: 45644442A/G
7-4: 45697418G/A
7-4: 45735767A/G
7-4: 45742026A/G
7-4: 45742413C/T
7-4: 45742525C/T
7-4: 45742808A/G
7-4: 45742951C/T
7-4: 45748828A/G
7-4: 45758854G/A
7-4: 45774025G/A
7-4: 45801907A/T
7-4: 45835549T/A
7-4: 45857032T/C
7-4: 45910476C/T
7-4: 45910601A/T
7-4: 45921057A/G
7-4: 45967229T/C
7-4: 46029432G/T
7-4: 46039806C/T
7-4: 46070912T/G
7-4: 46100475C/T
7-4: 46144493T/C
7-4: 46171622T/C
7-4: 46206917G/A
7-4: 46234146T/G
7-4: 46303194A/C
7-4: 46344981A/G
7-4: 46377812G/A
7-4: 46426651T/C
7-4: 46481441A/G
7-4: 46525570T/G
7-4: 46614346C/A
7-4: 46624954T/G
7-4: 46654208C/T
7-4: 46654222C/T
7-4: 46654316C/T
7-4: 46654361G/T
7-4: 46654412G/A
7-4: 46654424C/G
7-4: 46654435G/C
7-4: 46654463T/G
7-4: 46654655A/T
7-4: 46660805T/G
7-4: 46681092A/G
7-4: 46687514G/T
7-4: 46687622T/.
7-4: 46707421G/C
7-4: 46735575T/G
7-4: 46745419T/G
7-4: 46749410G/A
7-4: 46801216A/T
7-4: 46886192C/A
7-4: 46934513C/T
7-4: 46997193A/G
7-4: 47110186C/G
7-4: 47112457T/C
7-4: 47114925G/C
7-4: 47115248C/G
7-4: 47166128T/C
7-4: 47190295C/G
7-4: 47204879C/T
7-4: 47209037T/C
7-4: 47209232A/C
7-4: 47233869G/A
7-4: 47310803T/C
7-4: 47404935A/G
7-4: 47552276T/C
7-4: 47611697G/A
7-4: 47613849G/A
7-4: 47684271T/C
7-4: 47800360C/T
7-4: 47861391A/T
7-4: 47957712G/A
7-4: 47994646T/C
7-4: 48030615T/C
7-4: 48079726T/C
7-4: 48245152T/C
7-4: 48366456T/C
7-4: 48414152G/A
7-4: 48453657T/C
7-4: 48486335T/A
7-4: 48554374G/T
7-4: 48672027G/T
7-4: 48735271A/G
7-4: 48807028G/A
7-4: 48836774A/G
7-4: 48879102T/C
7-4: 48924891A/C
7-4: 48925109G/C
7-4: 48978276C/G
7-4: 49048976G/C
7-4: 49181170C/T
7-4: 49237700T/G
7-4: 49275756T/C
7-4: 49312710A/G
7-4: 49381873C/G
7-4: 49417231T/A
7-4: 49493205G/A
7-4: 49600229T/C
7-4: 49638055A/C
7-4: 49701608G/T
7-4: 49764790C/T
7-4: 49895784A/G
7-4: 49896886C/G
7-4: 49899353G/A
7-4: 49980504T/C
7-4: 50024497G/A
7-4: 50086604C/T
7-4: 50193185G/A
7-4: 50241083T/C
7-4: 50273234G/A
7-4: 50276209T/C
7-4: 50340929A/G
7-4: 50382407G/A
7-4: 50469191G/A
7-4: 50506684C/A
7-4: 50540274T/C
7-4: 50590148G/A
7-4: 50740192C/A
7-4: 50864488G/A
7-4: 50866553T/C
7-4: 50875486C/T
7-4: 50901305C/T
7-4: 51000851A/G
7-4: 51036107G/A
7-4: 51058175C/T
7-4: 51134484G/T
7-4: 51147542G/A
7-4: 51200219C/T
7-4: 51283398A/G
7-4: 51432550G/A
7-4: 51511144A/C
7-4: 51615125T/G
7-4: 51681500A/G
7-4: 51745920A/G
7-4: 51775079C/G
7-4: 51804006A/T
7-4: 51830002G/A
7-4: 52017044A/T
7-4: 52031876C/T
7-4: 52082250T/C
7-4: 52086158T/C
7-4: 52148419T/G
7-4: 52150587T/C
7-4: 52191233A/T
7-4: 52202440A/T
7-4: 52225624T/C
7-4: 52294572C/A
7-4: 52377572T/A
7-4: 52421216C/T
7-4: 52446319A/G
7-4: 52474284T/C
7-4: 52618658T/G
7-4: 52635520A/G
7-4: 52695864A/G
7-4: 52798840T/C
7-4: 52852583G/A
7-4: 52870636A/T
7-4: 52942392C/T
7-4: 52991332T/A
7-4: 53044534C/T
7-4: 53104675A/C
7-4: 53182856A/G
7-4: 53266165T/C
7-4: 53310714C/G
7-4: 53337057T/C
7-4: 53362708G/A
7-4: 53365425T/A
7-4: 53420875C/T
7-4: 53466470C/T
7-4: 53537130T/G
7-4: 53621609A/T
7-4: 53673845G/T
7-4: 53684816A/G
7-4: 53726111T/C
7-4: 53760314C/T
7-4: 53809851T/A
7-4: 53906717T/C
7-4: 53915597T/A
7-4: 53916911T/C
7-4: 53931606C/T
7-4: 53978810C/A
7-4: 54030492G/A
7-4: 54031218A/G
7-4: 54218516C/T
7-4: 54237081T/C
7-4: 54249814C/T
7-4: 54268708C/T
7-4: 54277822A/T
7-4: 54318213A/G
7-4: 54353618G/T
7-4: 54403156T/C
7-4: 54422303A/G
7-4: 54441828G/T
7-4: 54442399G/A
7-4: 54474871T/C
7-4: 54476677C/T
7-4: 54476808A/G
7-4: 54506536T/C
7-4: 54589481A/T
7-4: 54618317A/G
7-4: 54672692C/T
7-4: 54758251T/C
7-4: 54920832G/A
7-4: 54965328C/T
7-4: 55031784T/C
7-4: 55078894G/A
7-4: 55169279G/A
7-4: 55197552A/T
7-4: 55240404C/G
7-4: 55271517C/A
7-4: 55313891A/G
7-4: 55327683C/T
7-4: 55361244A/G
7-4: 55392466G/A
7-4: 55485148C/T
7-4: 55573800C/A
7-4: 55625431C/A
7-4: 55754142C/T
7-4: 55769127T/C
7-4: 55805862G/A
7-4: 55882705T/G
7-4: 56155343T/A
7-4: 56305012C/T
7-4: 56354262T/A
7-4: 56371680A/G
7-4: 56577581C/A
7-4: 56643604C/T
7-4: 56721628G/A
7-4: 56725917C/A
7-4: 56810915G/A
7-4: 56877437C/A
7-4: 56933092A/G
7-4: 57003932A/G
7-4: 57051748A/G
7-4: 57096057C/T
7-4: 57167970C/T
7-4: 57304411A/G
7-4: 57316914T/C
7-4: 57353515C/T
7-4: 57379183G/A
7-4: 57418436G/A
7-4: 57468234C/A
7-4: 57506160A/G
7-4: 57617767C/T
7-4: 57619710A/C
7-4: 57655759C/T
7-4: 57713925G/T
7-4: 57715217C/T
7-4: 57778893G/A
7-4: 57873559G/A
7-4: 58132879G/A
7-4: 58164807A/C
7-4: 58302290C/T
7-4: 58492031A/G
7-4: 58517867C/T
7-4: 58551500C/G
7-4: 58761097A/T
7-4: 58846392A/G
7-4: 58878134C/T
7-4: 58929453A/T
7-4: 59136545C/A
7-4: 59193935T/A
7-4: 59280084G/A
7-4: 59295978C/T
7-4: 59311076T/C
7-4: 59328570G/A
7-4: 59341365A/G
7-4: 59380205G/A
7-4: 59390514G/A
7-4: 59454919C/T
7-4: 59621256A/T
7-4: 59817114C/T
7-4: 59874658T/G
7-4: 60076511A/G
7-4: 60088641A/G
7-4: 60114914T/C
7-4: 60151841T/C
7-4: 60159917C/A
7-4: 60265244G/A
7-4: 60436935T/A
7-4: 60510433G/A
7-4: 60565448C/G
7-4: 60703377G/A
7-4: 60707186G/A
7-4: 60739954G/A
7-4: 60796407T/G
7-4: 60990654T/C
7-4: 61074540A/G
7-4: 61124745T/C
7-4: 61247958T/G
7-4: 61363991A/G
7-4: 61401546C/T
7-4: 61502108A/T
7-4: 61569912A/T
7-4: 61799580T/C
7-4: 61880786A/G
7-4: 61990253C/A
7-4: 62046881C/T
7-4: 62250705C/T
7-4: 62395812A/G
7-4: 62617101T/C
7-4: 62662807T/C
7-4: 62703503C/T
7-4: 62739048T/C
7-4: 62740030T/A
7-4: 62767709T/G
7-4: 62768527C/T
7-4: 62769986C/A
7-4: 62772570G/A
7-4: 62772948G/A
7-4: 62778250A/G
7-4: 62779012C/A
7-4: 62784106C/T
7-4: 62829006G/A
7-4: 63062903T/C
7-4: 63427158A/T
7-4: 63428228A/G
7-4: 63442690C/A
7-4: 63500967G/A
7-4: 63752510T/C
7-4: 63912556A/G
7-4: 63981013C/G
7-4: 64112251C/T
8-1: 97136T/C
8-1: 142650C/G
8-1: 359609A/C
8-1: 504596T/C
8-1: 558004A/C
8-1: 737666T/C
8-1: 753143A/T
8-1: 791866C/T
8-1: 806025G/A
8-1: 877154A/C
8-1: 877173G/C
8-1: 877196G/A
8-1: 877252C/T
8-1: 877260G/T
8-1: 877321C/T
8-1: 877497C/T
8-1: 877920A/G
8-1: 1004982A/G
8-1: 1255184A/C
8-1: 1308765A/T
8-1: 1399741G/A
8-1: 1503486A/G
8-1: 1505724A/T
8-1: 1513399C/T
8-1: 1751780A/C
8-1: 1786225C/T
8-1: 1933465A/C
8-1: 2048293C/T
8-1: 2062125T/A
8-1: 2203400C/T
8-1: 2275409T/A
8-1: 2278441A/C
8-1: 2291586C/G
8-1: 2478348C/A
8-1: 2622869T/C
8-1: 2644243C/T
8-1: 2655982A/G
8-1: 2658776T/C
8-1: 2795646T/G
8-1: 3086468A/G
8-1: 3110751C/G
8-1: 3136046G/C
8-1: 3221837G/A
8-1: 3252602T/C
8-1: 3413235T/A
8-1: 3523937T/C
8-1: 3566385C/A
8-1: 3769033G/A
8-1: 3780290G/A
8-1: 3854437T/A
8-1: 3897086G/T
8-1: 3977508A/T
8-1: 4049477T/C
8-1: 4068323C/A
8-1: 4086023G/A
8-1: 4124638C/T
8-1: 4145572G/T
8-1: 4396609A/C
8-1: 4560877A/G
8-1: 4569337C/T
8-1: 4570087C/A
8-1: 4570300C/A
8-1: 4570324C/A
8-1: 4579383C/T
8-1: 4602062G/T
8-1: 4621489T/C
8-1: 4660446C/T
8-1: 4691033C/T
8-1: 4716455C/A
8-1: 4806964T/C
8-1: 5047690G/T
8-1: 5143373A/T
8-1: 5191499G/A
8-1: 5227462G/A
8-1: 5383359A/G
8-1: 5442367T/A
8-1: 5503018T/G
8-1: 5528554G/A
8-1: 5605761T/A
8-1: 5804340A/G
8-1: 5882146T/C
8-1: 5962937C/G
8-1: 5965754T/C
8-1: 6011752C/T
8-1: 6244988C/T
8-1: 6397906G/A
8-1: 6447531T/A
8-1: 6465340C/T
8-1: 6485562C/T
8-1: 6508310G/A
8-1: 6544158C/A
8-1: 6565184C/T
8-1: 6641214G/A
8-1: 6685217T/A
8-1: 6726125A/T
8-1: 6729998A/G
8-1: 6871764A/G
8-1: 6886264T/A
8-1: 6935142A/G
8-1: 6951014C/T
8-1: 7127903T/A
8-1: 7364925T/G
8-1: 7366761G/T
8-1: 7402722T/C
8-1: 7416340A/T
8-1: 7426671T/G
8-1: 7486578T/C
8-1: 7501358C/A
8-1: 7534330C/A
8-1: 7551422A/G
8-1: 7572649G/T
8-1: 7577373T/C
8-1: 7578625T/C
8-1: 7578655G/A
8-1: 7578818C/T
8-1: 7579097A/G
8-1: 7579831A/G
8-1: 7588682G/A
8-1: 7611323A/T
8-1: 7651104G/A
8-1: 7773827T/A
8-1: 7791758T/C
8-1: 7832306C/T
8-1: 7989112C/T
8-1: 8000848T/A
8-1: 8070158T/A
8-1: 8139683T/C
8-1: 8147265A/G
8-1: 8174715T/C
8-1: 8187363C/T
8-1: 8255226G/A
8-1: 8280478T/C
8-1: 8297961C/T
8-1: 8314698T/C
8-1: 8466648T/A
8-1: 8582786T/A
8-1: 8605955C/A
8-1: 8840588G/A
8-1: 8859309A/T
8-1: 8891101C/A
8-1: 8912006G/C
8-1: 8943120C/A
8-1: 8977028A/T
8-1: 9036791A/C
8-1: 9056875A/T
8-1: 9078324G/C
8-1: 9200773T/C
8-1: 9236330C/T
8-1: 9281597A/G
8-1: 9327382G/C
8-1: 9357063G/T
8-1: 9384384C/T
8-1: 9416210G/A
8-1: 9418607G/A
8-1: 9480431A/C
8-1: 9491420C/T
8-1: 9492808A/G
8-1: 9500300T/C
8-1: 9513536A/C
8-1: 9532593T/A
8-1: 9544506A/T
8-1: 9571248T/A
8-1: 9597545T/C
8-1: 9637467G/A
8-1: 9645218A/T
8-1: 9645231A/G
8-1: 9648136G/A
8-1: 9649541T/A
8-1: 9649660T/A
8-1: 9663623C/T
8-1: 9733932A/G
8-1: 9736169A/T
8-1: 9776975A/G
8-1: 9806279C/T
8-1: 9833143G/T
8-1: 9882150C/A
8-1: 9893476A/C
8-1: 9920130A/G
8-1: 9944580C/T
8-1: 9984398G/A
8-1: 10002808A/C
8-1: 10057210C/T
8-1: 10077037C/A
8-1: 10102503T/C
8-1: 10171859G/C
8-1: 10189453A/G
8-1: 10271916T/C
8-1: 10272944T/G
8-1: 10295017C/T
8-1: 10384538C/G
8-1: 10473202T/A
8-1: 10499026G/A
8-1: 10599335C/T
8-1: 10630912A/G
8-1: 10658911T/G
8-1: 10719715T/A
8-1: 10731888T/C
8-1: 10763534A/G
8-1: 10795863G/A
8-1: 10821390A/G
8-1: 10850693A/G
8-1: 10888562C/T
8-1: 10945266T/C
8-1: 10945978C/T
8-1: 10950014A/T
8-1: 10968064C/T
8-1: 10969221C/T
8-1: 10970136C/T
8-1: 10975805T/C
8-1: 10975811A/T
8-1: 10976012A/G
8-1: 10986125G/T
8-1: 10986738T/A
8-1: 10986793G/A
8-1: 11015131T/G
8-1: 11049853G/A
8-1: 11078290C/A
8-1: 11145561C/G
8-1: 11188796T/C
8-1: 11220847T/G
8-1: 11254888A/T
8-1: 11357026A/T
8-1: 11448293T/C
8-1: 11533460A/T
8-1: 11543599A/T
8-1: 11579405A/G
8-1: 11629375T/A
8-1: 11683772G/A
8-1: 11711825G/A
8-1: 11756546C/T
8-1: 11768721C/T
8-1: 11801471T/G
8-1: 11813332C/T
8-1: 11833487A/G
8-1: 11894440T/G
8-1: 11917799T/C
8-1: 12030075C/G
8-1: 12204568A/G
8-1: 12223180A/C
8-1: 12277731T/C
8-1: 12357695C/T
8-1: 12440604C/G
8-1: 12468939C/T
8-1: 12471001G/A
8-1: 12505593A/T
8-1: 12572418C/A
8-1: 12589321A/G
8-1: 12591503T/A
8-1: 12592455G/A
8-1: 12658617T/C
8-1: 12658732T/C
8-1: 12684681C/T
8-1: 12695040A/T
8-1: 12719971C/A
8-1: 12856992G/A
8-1: 12993944T/G
8-1: 13015271C/A
8-1: 13035555T/C
8-1: 13055816A/C
8-1: 13196429T/G
8-1: 13218603T/C
8-1: 13270316G/A
8-1: 13339815C/T
8-1: 13379535A/T
8-1: 13460386T/C
8-1: 13593937C/A
8-1: 13776757A/C
8-1: 13857088C/T
8-1: 13916349G/A
8-1: 13985403T/C
8-1: 14020068A/T
8-1: 14039681T/C
8-1: 14074649A/G
8-1: 14081002C/G
8-1: 14082163G/A
8-1: 14082368A/G
8-1: 14085074C/T
8-1: 14085188G/C
8-1: 14114316T/C
8-1: 14166281T/A
8-1: 14193418A/G
8-1: 14288151G/A
8-1: 14311439A/C
8-1: 14382145G/A
8-1: 14485078G/A
8-1: 14633555A/C
8-1: 14671824T/A
8-1: 14689815A/G
8-1: 14690423C/T
8-1: 14697583A/G
8-1: 14700932C/T
8-1: 14878863A/G
8-1: 14901456G/A
8-1: 14915652G/A
8-1: 14934543T/G
8-1: 14997931T/C
8-1: 15021903G/A
8-1: 15053305C/G
8-1: 15106486C/T
8-1: 15181118C/T
8-1: 15221396A/T
8-1: 15251992C/T
8-1: 15263546G/A
8-1: 15275554A/.
8-1: 15279417C/A
8-1: 15295381A/T
8-1: 15307652A/T
8-1: 15310212T/A
8-1: 15320618A/G
8-1: 15389790C/G
8-1: 15393724C/T
8-1: 15394462A/G
8-1: 15394659C/T
8-1: 15394832G/A
8-1: 15395039T/A
8-1: 15395156T/G
8-1: 15395359T/C
8-1: 15395466G/A
8-1: 15396276C/A
8-1: 15396409G/C
8-1: 15396479A/G
8-1: 15397252A/G
8-1: 15404260A/G
8-1: 15418701G/A
8-1: 15434297A/C
8-1: 15434398A/G
8-1: 15435880C/T
8-1: 15436087A/C
8-1: 15447407T/A
8-1: 15448550T/A
8-1: 15456540A/T
8-1: 15457948T/C
8-1: 15497289A/T
8-1: 15498238C/A
8-1: 15513428C/A
8-1: 15513863T/C
8-1: 15513986T/C
8-1: 15527846G/A
8-1: 15529518T/C
8-1: 15529531A/G
8-1: 15554031T/G
8-1: 15566150A/C
8-1: 15592727A/G
8-1: 15608997A/T
8-1: 15620000G/A
8-1: 15647268A/T
8-1: 15650787G/A
8-1: 15674156T/C
8-1: 15705468T/A
8-1: 15721889T/A
8-1: 15723321G/T
8-1: 15731128C/A
8-1: 15743654C/G
8-1: 15755274T/A
8-1: 15755380C/A
8-1: 15771112C/A
8-1: 15783726A/G
8-1: 15795012C/T
8-1: 15795240C/T
8-1: 15808112C/T
8-1: 15838173G/C
8-1: 15839408T/C
8-1: 15840278C/T
8-1: 15850045A/G
8-1: 15852734C/T
8-1: 15854491A/G
8-1: 15854610T/C
8-1: 15864069T/A
8-1: 15869444G/C
8-1: 15870426C/T
8-1: 15870566C/A
8-1: 15876049G/A
8-1: 15887306T/G
8-1: 15908168T/C
8-1: 15916644T/C
8-1: 15916691T/A
8-1: 15916775C/T
8-1: 15918870T/A
8-1: 15930867T/C
8-1: 15931283T/C
8-1: 15935578T/C
8-1: 15944228A/G
8-1: 15961944A/G
8-1: 16024011C/A
8-1: 16039675G/A
8-1: 16049924C/T
8-1: 16072675G/A
8-1: 16088781G/A
8-1: 16104478A/G
8-1: 16112575C/T
8-1: 16112686C/G
8-1: 16116081A/G
8-1: 16138263C/A
8-1: 16142583T/C
8-1: 16156303T/G
8-1: 16184780T/A
8-1: 16215663A/G
8-1: 16226038C/T
8-1: 16236891C/A
8-1: 16254303A/G
8-1: 16256031G/T
8-1: 16269735A/G
8-1: 16280031T/A
8-1: 16290690C/G
8-1: 16294424G/C
8-1: 16294451T/C
8-1: 16294824T/.
8-1: 16301777A/C
8-1: 16317609T/A
8-1: 16333069T/A
8-1: 16349027C/T
8-1: 16360243T/C
8-1: 16371521A/C
8-1: 16387100C/T
8-1: 16401222C/A
8-1: 16440468T/C
8-1: 16503385C/T
8-1: 16531156A/T
8-1: 16559632A/C
8-1: 16573207A/T
8-1: 16586756G/A
8-1: 16620513C/A
8-1: 16646427A/G
8-1: 16672757T/C
8-1: 16687779C/T
8-1: 16692082G/A
8-1: 16703638A/C
8-1: 16716389G/A
8-1: 16730689C/T
8-1: 16762251T/C
8-1: 16789516G/C
8-1: 16800447A/G
8-1: 16822371A/T
8-1: 16835075T/A
8-1: 16869035G/T
8-1: 16894643A/G
8-1: 16916693C/A
8-1: 16920244A/T
8-1: 16975324C/T
8-1: 17005953G/C
8-1: 17006061G/A
8-1: 17006672T/A
8-1: 17058553G/C
8-1: 17100293G/A
8-1: 17127205A/C
8-1: 17166334T/G
8-1: 17177233C/T
8-1: 17197716G/A
8-1: 17205087T/C
8-1: 17260887G/A
8-1: 17292887A/G
8-1: 17336700A/T
8-1: 17337539A/T
8-1: 17338672T/G
8-1: 17338702T/A
8-1: 17375287G/A
8-1: 17378406G/A
8-1: 17386256G/T
8-1: 17416520A/G
8-1: 17417494T/A
8-1: 17418664T/C
8-1: 17419428C/T
8-1: 17420041G/T
8-1: 17420191G/A
8-1: 17420319T/A
8-1: 17420529C/A
8-1: 17420640A/T
8-1: 17420678T/C
8-1: 17420791C/T
8-1: 17421086G/A
8-1: 17421327C/T
8-1: 17421357G/T
8-1: 17421943C/A
8-1: 17421998A/G
8-1: 17422366A/T
8-1: 17422499G/T
8-1: 17422564G/A
8-1: 17422601C/T
8-1: 17422793G/A
8-1: 17423167T/C
8-1: 17444287G/A
8-1: 17444505A/G
8-1: 17444770G/T
8-1: 17444834A/G
8-1: 17445020C/T
8-1: 17494421A/T
8-1: 17578451C/T
8-1: 17589989T/C
8-1: 17612650G/T
8-1: 17732746G/A
8-1: 17734304A/G
8-1: 17800461G/T
8-1: 17877056C/T
8-1: 17903037T/C
8-1: 17999630T/G
8-1: 18017785C/T
8-1: 18085632A/T
8-1: 18097964C/T
8-1: 18206493T/A
8-1: 18250701G/A
8-1: 18250710G/A
8-1: 18255259T/C
8-1: 18265004A/C
8-1: 18373492C/G
8-1: 18382042A/C
8-1: 18398946A/C
8-1: 18413337C/T
8-1: 18423676T/C
8-1: 18434282C/T
8-1: 18457394T/A
8-1: 18492072C/G
8-1: 18576562A/G
8-1: 18626538T/C
8-1: 18667836T/C
8-1: 18699421A/G
8-1: 18753909T/C
8-1: 18870288G/T
8-1: 18874844C/T
8-1: 18874860G/T
8-1: 18896115G/A
8-1: 18916797C/T
8-1: 18937258G/A
8-1: 18941221A/G
8-1: 18991274T/A
8-1: 19011337A/G
8-1: 19023062T/C
8-1: 19061907T/A
8-1: 19098554A/G
8-1: 19114805A/G
8-1: 19140719T/A
8-1: 19293888T/G
8-1: 19312445A/T
8-1: 19331197A/C
8-1: 19346600A/T
8-1: 19422255C/T
8-1: 19448704G/C
8-1: 19551636T/C
8-1: 19581743T/G
8-1: 19617573G/T
8-1: 19636328C/G
8-1: 19645601A/.
8-1: 19655538C/A
8-1: 19706665G/A
8-1: 19749659C/A
8-1: 19785570G/A
8-1: 19851240C/G
8-1: 19861575C/T
8-1: 19893026C/A
8-1: 19943061A/T
8-1: 19990980T/G
8-1: 20200954C/T
8-1: 20241969A/G
8-1: 20290922G/A
8-1: 20340256T/A
8-1: 20351927G/T
8-1: 20353204C/T
8-1: 20387034T/C
8-1: 20400288T/C
8-1: 20415484T/C
8-1: 20497378C/A
8-1: 20526526T/G
8-1: 20539497T/C
8-1: 20565666A/T
8-1: 20566750T/A
8-1: 20567555G/A
8-1: 20569045G/A
8-1: 20569246T/G
8-1: 20583028G/T
8-1: 20583157T/C
8-1: 20604546C/T
8-1: 20665255T/G
8-1: 20683488G/T
8-1: 20805443C/T
8-1: 20836440C/T
8-1: 20865448G/A
8-1: 20865576C/T
8-1: 20876603A/G
8-1: 20894366G/A
8-1: 20910197C/T
8-1: 20936556G/T
8-1: 20982252C/T
8-1: 21001364T/C
8-1: 21064992A/C
8-1: 21090304A/G
8-1: 21092139G/A
8-1: 21224762A/T
8-1: 21263619G/A
8-1: 21276834C/T
8-1: 21405575T/G
8-1: 21481375G/A
8-1: 21561397T/A
8-1: 21647398A/G
8-1: 21702499G/A
8-1: 21739728G/A
8-1: 21819250T/A
8-1: 21882532G/A
8-1: 21883450A/C
8-1: 21897399G/C
8-1: 21913171G/C
8-1: 21972020T/A
8-1: 22003905A/C
8-1: 22023236C/T
8-1: 22090396G/A
8-1: 22239229T/C
8-1: 22259029G/A
8-1: 22266838C/A
8-1: 22439922A/G
8-1: 22465981G/C
8-1: 22477067A/T
8-1: 22478730G/T
8-1: 22564888G/A
8-1: 22574111C/T
8-1: 22591055C/T
8-1: 22618407C/T
8-1: 22650457T/A
8-1: 22714793T/C
8-1: 22747306C/T
8-1: 22805316C/G
8-1: 22811082G/A
8-1: 22843566A/C
8-1: 22867110T/C
8-1: 22923564A/T
8-1: 22963082G/A
8-1: 23047138T/A
8-1: 23071221T/A
8-1: 23103783C/T
8-1: 23146201G/A
8-1: 23159849C/T
8-1: 23162778T/C
8-1: 23217928G/T
8-1: 23266695C/A
8-1: 23347003G/C
8-1: 23394492C/A
8-1: 23432305C/T
8-1: 23483381C/T
8-1: 23577755T/A
8-1: 23603904G/A
8-1: 23637909G/A
8-1: 23637936A/G
8-1: 23744193C/T
8-1: 23744246G/A
8-1: 23744437A/G
8-1: 23744708C/T
8-1: 23744711G/A
8-1: 23744773T/C
8-1: 23744951G/T
8-1: 23807099C/T
8-1: 23846740T/G
8-1: 23846990G/A
8-1: 23899602A/T
8-1: 23911545G/A
8-1: 23913593T/A
8-1: 23944143A/G
8-1: 24013610T/C
8-1: 24159706A/C
8-1: 24184167T/A
8-1: 24184179C/T
8-1: 24220581C/T
8-1: 24227337C/T
8-1: 24275099G/A
8-1: 24277006A/.
8-1: 24305309T/C
8-1: 24344260C/T
8-1: 24356669T/A
8-1: 24409613G/A
8-1: 24428245G/A
8-1: 24448725G/A
8-1: 24523686T/C
8-1: 24621410C/T
8-1: 24621569T/G
8-1: 24622314C/T
8-1: 24716556G/A
8-1: 24868624T/G
8-1: 24886413G/T
8-1: 25010221G/T
8-1: 25030283G/A
8-1: 25085156C/G
8-1: 25190419G/A
8-1: 25191468G/T
8-1: 25200537A/G
8-1: 25247157G/A
8-1: 25327996T/C
8-1: 25406957A/C
8-1: 25450376C/T
8-1: 25478079C/T
8-1: 25526078A/G
8-1: 25564534T/C
8-1: 25622125C/A
8-1: 25657159T/A
8-1: 25667495C/T
8-1: 25686321G/A
8-1: 25715868G/A
8-1: 25744879G/T
8-1: 25814902T/G
8-1: 25911842G/A
8-1: 25926441G/A
8-1: 25998014G/T
8-1: 26107500A/G
8-1: 26126122C/T
8-1: 26141707C/A
8-1: 26183258T/C
8-1: 26188030A/T
8-1: 26224477G/A
8-1: 26224654C/T
8-1: 26242289A/G
8-1: 26304074A/C
8-1: 26313377T/C
8-1: 26316232A/C
8-1: 26336559T/C
8-1: 26576609A/G
8-1: 26658629G/A
8-1: 26681820T/C
8-1: 26712070A/G
8-1: 26749059C/T
8-1: 26775237G/T
8-1: 26867588T/G
8-1: 26945053A/C
8-1: 26963276C/A
8-1: 27017497G/A
8-1: 27044616A/G
8-1: 27075880T/C
8-1: 27108433A/C
8-1: 27139343C/T
8-1: 27174702T/G
8-1: 27200377C/T
8-1: 27276821T/C
8-1: 27293367T/C
8-1: 27323091G/T
8-1: 27436952A/T
8-1: 27466771T/C
8-1: 27530108A/C
8-1: 27568684G/C
8-1: 27614346C/T
8-1: 27626785G/T
8-1: 27668381A/T
8-1: 27685161A/T
8-1: 27696744T/C
8-1: 27710989G/C
8-1: 27739887G/T
8-1: 27784114A/G
8-1: 27810413T/C
8-1: 27927649T/C
8-1: 27929964T/A
8-1: 27930146T/A
8-1: 27931287C/T
8-1: 27933712G/A
8-1: 27940053C/T
8-1: 27952391T/C
8-1: 27970148C/T
8-1: 27999282T/G
8-1: 28102339C/T
8-1: 28141221C/T
8-1: 28163331G/A
8-1: 28171147A/G
8-1: 28186230A/C
8-1: 28216374G/A
8-1: 28216889C/A
8-1: 28216893G/T
8-1: 28223297C/T
8-1: 28226846G/A
8-1: 28233938T/C
8-1: 28250025G/A
8-1: 28268173A/C
8-1: 28302465G/A
8-1: 28387146G/T
8-1: 28449717A/G
8-1: 28508761C/A
8-1: 28554243T/C
8-1: 28577631A/G
8-1: 28579445A/G
8-1: 28609099G/T
8-1: 28636091A/G
8-1: 28682208A/G
8-1: 28692594G/A
8-1: 28703715G/A
8-1: 28765987A/G
8-1: 28843050C/T
8-1: 28888864G/A
8-1: 28898183G/A
8-1: 28934241T/C
8-1: 28951024A/C
8-1: 29007605G/T
8-1: 29008013A/G
8-1: 29042538A/G
8-1: 29099998A/C
8-1: 29109920A/G
8-1: 29109921T/C
8-1: 29166944A/G
8-1: 29195866G/C
8-1: 29241290T/A
8-1: 29281045G/T
8-1: 29312239A/G
8-1: 29339460G/T
8-1: 29380107G/T
8-1: 29463464C/G
8-1: 29507454C/T
8-1: 29539910C/A
8-1: 29608046G/A
8-1: 29634830A/G
8-1: 29672673G/A
8-1: 29702595T/C
8-1: 29704942G/A
8-1: 29722262A/G
8-1: 29779979C/T
8-1: 29894871C/T
8-1: 29907393T/C
8-1: 29921163G/T
8-1: 29966510C/A
8-1: 30029110A/C
8-1: 30051937C/G
8-1: 30161724C/T
8-1: 30168004T/C
8-1: 30198014G/A
8-1: 30255024A/G
8-1: 30265137G/A
8-1: 30331983C/T
8-1: 30421325A/T
8-1: 30430422T/A
8-1: 30483328G/A
8-1: 30682471G/A
8-1: 30767587C/G
8-1: 30911080G/T
8-1: 30914184G/A
8-1: 30914896G/A
8-1: 30921518T/C
8-1: 31011285A/G
8-1: 31175409C/A
8-1: 31176762G/C
8-1: 31222227G/A
8-1: 31264754A/C
8-1: 31325428C/A
8-1: 31368973T/A
8-1: 31369322G/A
8-1: 31417296A/G
8-1: 31478115G/C
8-1: 31628356T/G
8-1: 31659696A/G
8-1: 31670826A/G
8-1: 31695683T/C
8-1: 31769189A/G
8-1: 31797994T/C
8-1: 31878728T/C
8-1: 31914330G/T
8-1: 31944996C/G
8-1: 31977442G/A
8-1: 32018985C/G
8-1: 32023544G/A
8-1: 32128021C/T
8-1: 32222548A/G
8-1: 32275123T/C
8-1: 32287575A/G
8-1: 32320710C/A
8-1: 32391556T/A
8-1: 32451967A/C
8-1: 32485072G/T
8-1: 32526468C/T
8-1: 32612865C/T
8-1: 32640758A/T
8-1: 32715956C/T
8-1: 32735541T/C
8-1: 32753803G/A
8-1: 32784157A/G
8-1: 32832120A/G
8-1: 32861009A/G
8-1: 32927711T/C
8-1: 32987182A/G
8-1: 33077602A/C
8-1: 33205216A/C
8-1: 33235814A/G
8-1: 33270609A/T
8-1: 33270930T/A
8-1: 33271087T/C
8-1: 33271163C/T
8-1: 33272191A/G
8-1: 33278938C/T
8-1: 33279043C/T
8-1: 33279254T/C
8-1: 33279476C/T
8-1: 33279508A/G
8-1: 33281004A/G
8-1: 33281312G/A
8-1: 33281622T/G
8-1: 33281819T/G
8-1: 33292500G/A
8-1: 33305094G/T
8-1: 33317670T/C
8-1: 33318089A/G
8-1: 33332051A/C
8-1: 33342066C/T
8-1: 33356551G/A
8-1: 33367157T/A
8-1: 33476791T/C
8-1: 33502347A/G
8-1: 33510009C/A
8-1: 33510284A/G
8-1: 33514237A/C
8-1: 33517031A/C
8-1: 33517263C/G
8-1: 33517445T/C
8-1: 33518149C/T
8-1: 33518591T/C
8-1: 33518667C/A
8-1: 33518851A/G
8-1: 33522106C/A
8-1: 33522281A/G
8-1: 33534817G/A
8-1: 33544863C/T
8-1: 33618834C/T
8-1: 33637752A/T
8-1: 33649202A/G
8-1: 33686912C/T
8-1: 33687187G/A
8-1: 33687431C/T
8-1: 33691526C/T
8-1: 33691660A/G
8-1: 33691671C/G
8-1: 33693169A/G
8-1: 33694229A/G
8-1: 33698139C/T
8-1: 33699116G/T
8-1: 33714419T/C
8-1: 33714555T/C
8-1: 33714560A/G
8-1: 33714563C/T
8-1: 33722359A/T
8-1: 33724577G/A
8-1: 33804457T/G
8-1: 33899653C/T
8-1: 33957030G/A
8-1: 33957146G/C
8-1: 33977026C/T
8-1: 34011472T/C
8-1: 34026658G/T
8-1: 34043114C/T
8-1: 34091243A/T
8-1: 34177730G/A
8-1: 34209130A/G
8-1: 34248519T/A
8-1: 34249180G/A
8-1: 34249362G/C
8-1: 34259729C/T
8-1: 34405555T/C
8-1: 34406079A/C
8-1: 34406165G/A
8-1: 34462447A/C
8-1: 34487541G/A
8-1: 34620071A/T
8-1: 34670770T/C
8-1: 34715845T/C
8-1: 34770748C/T
8-1: 34795147G/T
8-1: 34942984T/C
8-1: 35017596A/C
8-1: 35078779C/T
8-1: 35097487G/A
8-1: 35261032C/A
8-1: 35362256T/C
8-1: 35375824G/T
8-1: 35391375A/C
8-1: 35391806A/G
8-1: 35391909G/A
8-1: 35392299G/T
8-1: 35392402A/G
8-1: 35392463G/T
8-1: 35392519T/C
8-1: 35392766A/G
8-1: 35393138C/G
8-1: 35400665C/T
8-1: 35414967C/T
8-1: 35496400T/C
8-1: 35507084C/T
8-1: 35631317C/T
8-1: 35825954C/T
8-1: 35862278A/G
8-1: 35862970A/G
8-1: 35863029T/C
8-1: 35889324C/A
8-1: 36066102A/T
8-1: 36080733C/T
8-1: 36144766A/G
8-1: 36241333A/G
8-1: 36267253T/A
8-1: 36332381T/C
8-1: 36351663A/T
8-1: 36386982A/G
8-1: 36422411G/A
8-1: 36550728G/A
8-1: 36567958A/G
8-1: 36616238C/A
8-1: 36653117C/T
8-1: 36681705C/A
8-1: 36722388T/A
8-1: 36781599C/T
8-1: 36803251A/G
8-1: 36837957G/A
8-1: 36867708G/A
8-1: 36877958C/A
8-1: 36890044A/G
8-1: 36919044T/C
8-1: 36945292C/T
8-1: 36992020C/T
8-1: 37001969A/G
8-1: 37005554G/A
8-1: 37038119G/C
8-1: 37053118G/A
8-1: 37091671A/C
8-1: 37179962G/T
8-1: 37181290A/G
8-1: 37191988T/G
8-1: 37218424A/G
8-1: 37247711G/A
8-1: 37283885C/T
8-1: 37297831T/C
8-1: 37309164C/T
8-1: 37320827C/G
8-1: 37352423G/T
8-1: 37360653T/A
8-1: 37370724A/T
8-1: 37394190T/A
8-1: 37422298A/G
8-1: 37437271G/A
8-1: 37463148T/C
8-1: 37471431G/T
8-1: 37488932G/A
8-1: 37512923A/G
8-1: 37517509A/T
8-1: 37532559G/A
8-1: 37685416G/T
8-1: 37745544A/G
8-1: 37798517T/G
8-1: 37848288A/G
8-1: 37917345T/G
8-1: 37947821G/T
8-1: 37948034T/A
8-1: 37958179C/A
8-1: 38009134C/G
8-1: 38078019A/T
8-1: 38124593C/T
8-1: 38129303A/T
8-1: 38397646A/G
8-1: 38400469C/G
8-1: 38433647G/A
8-1: 38465088G/A
8-1: 38544200A/G
8-1: 38613996T/C
8-1: 38652275G/A
8-1: 38653801T/A
8-1: 38851731C/G
8-1: 38855252C/T
8-1: 38855318C/T
8-1: 38904772T/C
8-1: 38989215T/C
8-1: 39046308T/A
8-1: 39108959T/A
8-1: 39159350G/T
8-1: 39222562A/G
8-1: 39230144A/T
8-1: 39259380G/T
8-1: 39259696C/.
8-1: 39259697C/.
8-1: 39259698C/.
8-1: 39259705G/.
8-1: 39259719C/T
8-1: 39259727T/.
8-1: 39259735C/.
8-1: 39302396T/A
8-1: 39304089T/A
8-1: 39328061A/T
8-1: 39365282T/C
8-1: 39390475T/G
8-1: 39447177C/T
8-1: 39460651C/T
8-1: 39461858G/A
8-1: 39462971T/C
8-1: 39463600G/A
8-1: 39477470G/C
8-1: 39639412T/G
8-1: 39643590A/T
8-1: 39674644T/C
8-1: 39804354C/T
8-1: 39869073A/G
8-1: 39896973A/G
8-1: 39915459T/C
8-1: 39933873C/G
8-1: 39937785A/G
8-1: 40006522C/A
8-1: 40081082G/C
8-1: 40098855A/G
8-1: 40100159C/T
8-1: 40142933G/A
8-1: 40157094G/T
8-1: 40194346T/G
8-1: 40203158G/T
8-1: 40204711T/G
8-1: 40252905T/G
8-1: 40306928T/A
8-1: 40353076G/C
8-1: 40460035C/T
8-1: 40467745T/C
8-1: 40556754A/C
8-1: 40637955T/G
8-1: 40692156C/T
8-1: 40838051G/A
8-1: 40838057C/T
8-1: 40848210A/G
8-1: 40858734C/G
8-1: 40954305G/T
8-1: 41000089G/T
8-1: 41012452A/G
8-1: 41025575C/A
8-1: 41049827T/C
8-1: 41054732A/C
8-1: 41054942G/C
8-1: 41071159C/G
8-1: 41076428A/G
8-1: 41082647A/G
8-1: 41118360A/G
8-1: 41180575A/C
8-1: 41211684T/A
8-1: 41251498A/G
8-1: 41289626C/T
8-1: 41402835G/A
8-1: 41432724C/A
8-1: 41433036T/C
8-1: 41439288T/G
8-1: 41439398G/A
8-1: 41444140G/A
8-1: 41444247G/A
8-1: 41444528C/T
8-1: 41445714T/C
8-1: 41456028A/C
8-1: 41463918A/C
8-1: 41464608C/T
8-1: 41464615G/A
8-1: 41464771A/G
8-1: 41466867T/C
8-1: 41472646G/A
8-1: 41545909C/T
8-1: 41572603T/C
8-1: 41652638T/G
8-1: 41696273C/T
8-1: 41736918T/C
8-1: 41891199T/A
8-1: 41894035G/A
8-1: 41904859C/T
8-1: 41915066G/A
8-1: 41926727C/G
8-1: 42063724T/C
8-1: 42129872C/T
8-1: 42297878C/A
8-1: 42354798C/T
8-1: 42378761T/A
8-1: 42424543A/T
8-1: 42449410C/A
8-1: 42465864A/G
8-1: 42667803A/G
8-1: 42759634A/G
8-1: 42839730A/C
8-1: 42882253C/A
8-1: 42911668T/C
8-1: 42964008A/T
8-1: 43027346A/G
8-1: 43175984A/G
8-1: 43206752C/T
8-1: 43294910C/A
8-1: 43560391C/T
8-1: 43714394C/T
8-1: 43714730A/C
8-1: 43735524G/A
8-1: 43779922T/C
8-1: 43803740T/C
8-1: 43858310A/C
8-1: 43910767A/C
8-1: 43951774T/C
8-1: 44037580G/A
8-1: 44050457A/G
8-1: 44050565C/T
8-1: 44051766A/C
8-1: 44063697C/T
8-1: 44094022A/G
8-1: 44117194G/A
8-1: 44125789G/A
8-1: 44126208C/T
8-1: 44126414T/C
8-1: 44126464T/C
8-1: 44126583T/C
8-1: 44126750A/G
8-1: 44126958A/G
8-1: 44127259G/A
8-1: 44127308G/A
8-1: 44127554A/G
8-1: 44127555C/T
8-1: 44141843T/C
8-1: 44169911A/G
8-1: 44171485T/C
8-1: 44171520C/T
8-1: 44171610A/G
8-1: 44175820G/C
8-1: 44175981G/A
8-1: 44185235G/A
8-1: 44188797G/A
8-1: 44189205T/G
8-1: 44218580G/T
8-1: 44223107A/G
8-1: 44249497A/T
8-1: 44260421G/A
8-1: 44260524G/A
8-1: 44264828A/G
8-1: 44265032C/T
8-1: 44266379C/G
8-1: 44286757G/A
8-1: 44309826G/A
8-1: 44349330G/A
8-1: 44354794G/A
8-1: 44407165C/A
8-1: 44538405C/G
8-1: 44547734T/C
8-1: 44547740T/G
8-1: 44551153G/C
8-1: 44569633T/C
8-1: 44628404G/A
8-1: 44640073G/A
8-1: 44732984C/A
8-1: 44758911G/A
8-1: 44783554G/A
8-1: 44812168T/C
8-1: 44841054A/C
8-1: 44895295C/T
8-1: 44934714C/T
8-1: 44965869C/A
8-1: 44977347G/A
8-1: 45016604C/T
8-1: 45054849G/A
8-1: 45061655C/T
8-1: 45122013C/T
8-1: 45178253A/C
8-1: 45191496C/T
8-1: 45208490G/A
8-1: 45247916T/A
8-1: 45258883C/T
8-1: 45263603G/A
8-1: 45352657A/G
8-1: 45397515A/G
8-1: 45464780C/T
8-1: 45543347C/T
8-1: 45657775C/T
8-1: 45751242T/C
8-1: 45878528C/T
8-1: 45902946C/T
8-1: 45944028T/A
8-1: 45974155A/T
8-1: 46017490A/T
8-1: 46018097A/C
8-1: 46098631G/A
8-1: 46108851C/T
8-1: 46168425A/G
8-1: 46185510T/A
8-1: 46186265T/C
8-1: 46190944T/C
8-1: 46192930T/A
8-1: 46214578C/T
8-1: 46224921C/G
8-1: 46255762C/T
8-1: 46283935T/A
8-1: 46353762G/A
8-1: 46448514G/A
8-1: 46472041G/C
8-1: 46497959A/G
8-1: 46549658C/T
8-1: 46568326T/C
8-1: 46569346C/T
8-1: 46620273T/A
8-1: 46653948G/A
8-1: 46697998A/G
8-1: 46710620G/A
8-1: 46768081A/T
8-1: 46858013C/G
8-1: 46898554C/T
8-1: 46910069C/A
8-1: 46921564C/T
8-1: 46967323G/A
8-1: 47043723C/A
8-1: 47090288A/G
8-1: 47130433G/A
8-1: 47160053C/T
8-1: 47209798G/T
8-1: 47244359G/A
8-1: 47256399A/T
8-1: 47260513C/G
8-1: 47263619C/G
8-1: 47267173C/A
8-1: 47283869C/A
8-1: 47322952C/T
8-1: 47326388A/T
8-1: 47351338A/G
8-1: 47392212G/A
8-1: 47466462T/G
8-1: 47488735C/T
8-1: 47531083G/T
8-1: 47621129C/T
8-1: 47690140A/C
8-1: 47772672G/T
8-1: 47875919G/A
8-1: 47923156G/C
8-1: 47963497G/A
8-1: 48191517A/G
8-1: 48200703A/T
8-1: 48205001T/C
8-1: 48218930A/T
8-1: 48229978A/T
8-1: 48308596A/T
8-1: 48334044T/C
8-1: 48369607G/A
8-1: 48399846T/A
8-1: 48447892A/T
8-1: 48475757G/A
8-1: 48533122A/G
8-1: 48543950A/G
8-1: 48558776G/T
8-1: 48632106A/C
8-1: 48664213G/T
8-1: 48690234T/C
8-1: 48777546T/C
8-1: 48824100C/T
8-1: 48824612G/T
8-1: 48835023T/G
8-1: 48835063A/G
8-1: 48846233C/T
8-1: 48846885C/T
8-1: 48860819C/G
8-1: 48878341A/T
8-1: 48878958A/C
8-1: 48907780A/G
8-1: 48952049C/T
8-1: 49003890T/C
8-1: 49072396G/T
8-1: 49078272C/G
8-1: 49080650A/G
8-1: 49084771C/A
8-1: 49087165A/T
8-1: 49087854C/G
8-1: 49088536T/C
8-1: 49102281C/T
8-1: 49114800G/T
8-1: 49123067C/T
8-1: 49171236A/T
8-1: 49190214A/T
8-1: 49230833C/T
8-1: 49244604A/G
8-1: 49244831C/T
8-1: 49249323G/T
8-1: 49253413C/T
8-1: 49254992G/T
8-1: 49299151A/G
8-1: 49311950A/G
8-1: 49313861G/T
8-1: 49344206G/A
8-1: 49344388C/T
8-1: 49371519C/T
8-1: 49427275G/T
8-1: 49442214A/T
8-1: 49476568G/A
8-1: 49487914A/G
8-1: 49504145T/C
8-1: 49505390A/C
8-1: 49530367C/T
8-1: 49598907G/A
8-1: 49599300C/T
8-1: 49599634G/T
8-1: 49614469C/G
8-1: 49629434C/T
8-1: 49640011A/G
8-1: 49643806G/T
8-1: 49672665C/T
8-1: 49705488C/T
8-1: 49736768G/A
8-1: 49766005C/A
8-1: 49777927C/T
8-1: 49783303A/G
8-1: 49783468A/C
8-1: 49799821C/A
8-1: 49834944A/T
8-1: 49873839T/C
8-1: 49911225A/G
8-1: 49936274T/C
8-1: 49962537A/G
8-1: 49967844A/G
8-1: 49978276T/C
8-1: 49985378T/C
8-1: 49990307A/T
8-1: 50004273G/C
8-1: 50019238T/A
8-1: 50030981A/T
8-1: 50065613A/G
8-1: 50068869T/A
8-1: 50069126T/C
8-1: 50069276T/C
8-1: 50070490C/T
8-1: 50092466C/A
8-1: 50092882C/A
8-1: 50122785A/T
8-1: 50142404G/A
8-1: 50147644C/A
8-1: 50152688C/T
8-1: 50200572T/G
8-1: 50231275T/A
8-1: 50277218A/G
8-1: 50310121G/A
8-1: 50330640A/T
8-1: 50389806A/T
8-1: 50417281C/G
8-1: 50428274G/A
8-1: 50453223T/A
8-1: 50491740C/A
8-1: 50502020C/T
8-1: 50514421T/C
8-1: 50516654C/A
8-1: 50517113A/T
8-1: 50517192C/T
8-1: 50533985C/T
8-1: 50559554T/C
8-1: 50560467C/G
8-1: 50573132G/A
8-1: 50591738T/C
8-1: 50638234G/A
8-1: 50650430G/T
8-1: 50651379A/G
8-1: 50652226A/G
8-1: 50652250G/C
8-1: 50656140G/A
8-1: 50680102T/A
8-1: 50716176C/T
8-1: 50717577C/T
8-1: 50772614G/A
8-1: 50821021G/A
8-1: 50921074T/G
8-1: 50953496C/T
8-1: 50954019A/G
8-1: 51015330C/T
8-1: 51037375A/G
8-1: 51059998G/C
8-1: 51075096T/G
8-1: 51089529G/A
8-1: 51103406G/A
8-1: 51111288A/T
8-1: 51111330C/T
8-1: 51118733T/C
8-1: 51118736G/C
8-1: 51118740T/G
8-1: 51118746T/G
8-1: 51118780G/A
8-1: 51118786T/G
8-1: 51118809A/C
8-1: 51118865T/C
8-1: 51118886A/G
8-1: 51118909T/C
8-1: 51118952C/G
8-1: 51118993C/T
8-1: 51119285A/C
8-1: 51139794G/T
8-1: 51139870T/C
8-1: 51140120A/G
8-1: 51150957T/C
8-1: 51181946T/C
8-1: 51218956G/A
8-1: 51269587A/G
8-1: 51340960C/T
8-1: 51341112G/A
8-1: 51343097C/A
8-1: 51405040C/T
8-1: 51503500C/T
8-1: 51611700C/T
8-1: 51614058C/T
8-1: 51614413A/T
8-1: 51649813A/G
8-1: 51709520T/A
8-1: 51728030A/G
8-1: 51914535T/G
8-1: 51914877G/T
8-1: 51915128T/C
8-1: 51916340G/A
8-1: 51917127T/G
8-1: 51919495G/A
8-1: 51919618T/A
8-1: 51924299G/A
8-1: 51924417A/T
8-1: 51925326G/A
8-1: 51927410T/C
8-1: 51933883A/C
8-1: 51933891G/A
8-1: 51936536T/A
8-1: 52014724G/T
8-1: 52057051C/T
8-1: 52062624C/T
8-1: 52084475T/C
8-1: 52118824A/G
8-1: 52126976A/G
8-1: 52135026A/G
8-1: 52138241T/C
8-1: 52154729G/A
8-1: 52165062C/G
8-1: 52223372A/G
8-1: 52243139C/T
8-1: 52291425T/C
8-1: 52294398A/C
8-1: 52296334A/C
8-1: 52298098G/A
8-1: 52298114A/T
8-1: 52306953T/C
8-1: 52368957C/A
8-1: 52393828A/G
8-1: 52414973T/C
8-1: 52450480A/G
8-1: 52479226A/T
8-1: 52495167T/C
8-1: 52496916C/A
8-1: 52513409C/T
8-1: 52545778G/A
8-1: 52599229G/A
8-1: 52627153G/A
8-1: 52653733G/A
8-1: 52693974T/C
8-1: 52704308G/T
8-1: 52735803G/A
8-1: 52768062C/T
8-1: 52779806G/A
8-1: 52846393G/A
8-1: 52864824G/T
8-1: 52877615C/T
8-1: 52890433C/T
8-1: 52902039T/C
8-1: 52932661T/C
8-1: 52932964G/A
8-1: 52955969A/G
8-1: 52956752A/G
8-1: 52970691A/T
8-1: 53009410T/C
8-1: 53053811C/T
8-1: 53056359G/C
8-1: 53057259A/G
8-1: 53065391G/A
8-1: 53065430G/C
8-1: 53105923T/A
8-1: 53130072G/T
8-1: 53194239C/A
8-1: 53220374T/A
8-1: 53281608A/G
8-1: 53310115A/C
8-1: 53310380T/C
8-1: 53369840C/T
8-1: 53415336G/A
8-1: 53456159C/T
8-1: 53492385G/A
8-1: 53508643A/T
8-1: 53556480A/C
8-1: 53573246A/G
8-1: 53612675A/C
8-1: 53641387T/C
8-1: 53642957A/T
8-1: 53655638G/A
8-1: 53678237A/C
8-1: 53693787T/C
8-1: 53727417T/G
8-1: 53731348A/G
8-1: 53732943T/C
8-1: 53737796C/A
8-1: 53739769G/A
8-1: 53740695T/C
8-1: 53741302G/T
8-1: 53769762G/A
8-1: 53802953C/T
8-1: 53812711A/T
8-1: 53815136A/G
8-1: 53821427A/T
8-1: 53822431G/A
8-1: 53822551G/C
8-1: 53823461G/A
8-1: 53841356G/T
8-1: 53973553T/A
8-1: 53989923G/A
8-1: 53990273G/A
8-1: 53991636C/T
8-1: 54035091C/A
8-1: 54086954A/C
8-1: 54130328G/A
8-1: 54153184T/A
8-1: 54163414A/G
8-1: 54195906G/A
8-1: 54242291A/G
8-1: 54256517C/T
8-1: 54283841C/.
8-1: 54300199C/T
8-1: 54310256T/C
8-1: 54327605T/C
8-1: 54385032G/A
8-1: 54433836A/T
8-1: 54435733A/G
8-1: 54467222A/G
8-1: 54468252G/A
8-1: 54481802A/G
8-1: 54494234T/C
8-1: 54494239G/A
8-1: 54504400T/C
8-1: 54528379C/G
8-1: 54553383C/T
8-1: 54564816A/G
8-1: 54575798A/T
8-1: 54590027T/C
8-1: 54602066G/A
8-1: 54610401G/A
8-1: 54628760G/A
8-1: 54635597A/T
8-1: 54645711T/C
8-1: 54678800G/T
8-1: 54692657C/G
8-1: 54750147A/C
8-1: 54766921C/A
8-1: 54768826T/A
8-1: 54769999T/C
8-1: 54770125A/G
8-1: 54770229G/A
8-1: 54792058T/A
8-1: 54840967A/G
8-1: 54840987T/G
8-1: 54858991A/G
8-1: 54908983A/G
8-1: 54964196A/C
8-1: 55011329C/A
8-1: 55014451G/A
8-1: 55036923G/C
8-1: 55071819C/G
8-1: 55092460T/C
8-1: 55101021A/G
8-1: 55121803T/A
8-1: 55168112A/G
8-1: 55185073C/T
8-1: 55185086C/A
8-1: 55185315C/A
8-1: 55191445G/A
8-1: 55214303G/A
8-1: 55240642T/C
8-1: 55255636C/T
8-1: 55261805C/T
8-1: 55261906C/T
8-1: 55261907G/A
8-1: 55277387A/G
8-1: 55317417G/A
8-1: 55355750A/T
8-1: 55382231G/A
8-1: 55413646G/A
8-1: 55501520C/T
8-1: 55558549T/C
8-1: 55632055G/A
8-1: 55660945G/A
8-1: 55724224G/C
8-1: 55753712G/A
8-1: 55839379G/A
8-1: 55935224G/A
8-1: 55981194C/G
8-1: 56046869A/G
8-1: 56062080C/T
8-1: 56066866A/G
8-1: 56076679A/G
8-1: 56117720C/T
8-1: 56192507A/G
8-1: 56234804T/G
8-1: 56272612C/T
8-1: 56283276T/C
8-1: 56310196G/T
8-1: 56353401T/A
8-1: 56401856G/A
8-1: 56429568C/T
8-1: 56473065G/A
8-1: 56496904G/C
8-1: 56506941C/T
8-1: 56522136G/A
8-1: 56528432T/A
8-1: 56528464C/T
8-1: 56530910G/A
8-1: 56533521C/T
8-1: 56533841C/T
8-1: 56534046G/T
8-1: 56545581C/A
8-1: 56548300C/T
8-1: 56584446T/A
8-1: 56598479A/C
8-1: 56621363A/G
8-1: 56637727C/A
8-1: 56642623T/C
8-1: 56674033A/G
8-1: 56684748G/A
8-1: 56716506A/G
8-1: 56813560A/T
8-1: 56834536C/G
8-1: 56862532G/A
8-1: 56902079A/C
8-1: 56927277A/C
8-1: 56957810C/T
8-1: 56958834A/G
8-1: 56988395C/T
8-1: 56991064C/A
8-1: 57000325T/C
8-1: 57011008G/T
8-1: 57029085G/A
8-1: 57077547A/T
8-1: 57088867T/C
8-1: 57103457T/G
8-1: 57110587T/A
8-1: 57110699A/G
8-1: 57111853C/A
8-1: 57111879C/A
8-1: 57114889G/A
8-1: 57128584C/A
8-1: 57180399C/T
8-1: 57218191T/C
8-1: 57256834G/C
8-1: 57271863A/G
8-1: 57285560T/C
8-1: 57312911G/A
8-1: 57369621A/G
8-1: 57398462G/T
8-1: 57403168A/T
8-1: 57403207G/C
8-1: 57416403A/T
8-1: 57416515G/C
8-1: 57431636C/T
8-1: 57460188C/T
8-1: 57475284C/T
8-1: 57508474T/A
8-1: 57510497G/A
8-1: 57544715A/C
8-1: 57563878A/C
8-1: 57615255C/T
8-1: 57667841A/G
8-1: 57724367A/T
8-1: 57728971C/G
8-1: 57728997G/T
8-1: 57729374T/G
8-1: 57729520C/A
8-1: 57729964T/C
8-1: 57854294A/C
8-1: 57869804A/G
8-1: 57927455A/G
8-1: 58006678C/T
8-1: 58019378A/T
8-1: 58031924A/G
8-1: 58061471G/A
8-1: 58091494G/A
8-1: 58105750C/G
8-1: 58120173A/C
8-1: 58146556G/A
8-1: 58205281T/A
8-1: 58233460A/C
8-1: 58264567T/C
8-1: 58306111C/A
8-1: 58404271C/A
8-1: 58408162C/T
8-1: 58432396G/A
8-1: 58518551T/C
8-1: 58521929A/G
8-1: 58529347C/T
8-1: 58530218G/T
8-1: 58531603C/T
8-1: 58531794G/C
8-1: 58545667T/C
8-1: 58590961T/C
8-1: 58680665G/A
8-1: 58690720T/G
8-1: 58753002C/T
8-1: 58847965A/G
8-1: 58903236T/C
8-1: 58913915T/C
8-1: 58993011C/T
8-1: 59011170C/T
8-1: 59030118G/C
8-1: 59085488T/A
8-1: 59157105T/C
8-1: 59194252C/T
8-1: 59228920C/G
8-1: 59277069C/T
8-1: 59328513C/A
8-1: 59351647C/T
8-1: 59352191A/T
8-1: 59352334C/T
8-1: 59352400A/G
8-1: 59353881G/A
8-1: 59369815T/G
8-1: 59395867T/C
8-1: 59428698A/T
8-1: 59471367C/A
8-1: 59505050C/T
8-1: 59559521C/T
8-1: 59668879A/T
8-1: 59690544G/A
8-1: 59700899A/C
8-1: 59733445G/A
8-1: 59865368G/C
8-1: 59876020T/C
8-1: 59924457A/G
8-1: 60072898C/T
8-1: 60098736T/C
8-1: 60267587C/A
8-1: 60308438A/G
8-1: 60352643C/A
8-1: 60428580G/T
8-1: 60450920A/G
8-1: 60491043C/T
8-1: 60522228T/C
8-1: 60663453A/G
8-1: 60699715G/C
8-1: 60734831C/G
8-1: 60745218A/C
8-1: 60789461T/C
8-1: 60880531T/G
8-1: 60937928G/C
8-1: 60992507C/T
8-1: 61007775T/C
8-1: 61184136A/G
8-1: 61223715T/G
8-1: 61265813G/A
8-1: 61266947T/A
8-1: 61358685T/A
8-1: 61389930G/A
8-1: 61533984A/G
8-1: 61546764A/T
8-1: 61572647G/A
8-1: 61623383A/G
8-1: 61688117A/T
8-1: 61746549G/A
8-1: 61759244G/A
8-1: 61771897C/T
8-1: 61771936C/G
8-1: 61788580A/T
8-1: 61854719A/G
8-1: 61864966A/C
8-1: 61931284G/A
8-1: 61938667G/C
8-1: 61938816G/A
8-1: 61939560C/G
8-1: 61962997A/C
8-1: 61988779C/T
8-1: 62008277G/A
8-1: 62043995T/C
8-1: 62121573T/A
8-1: 62148645C/T
8-1: 62189806G/T
8-1: 62256888T/C
8-1: 62294165G/A
8-1: 62337040G/A
8-1: 62368002G/C
8-1: 62368354T/C
8-1: 62396634A/G
8-1: 62492691T/A
8-1: 62493499C/T
8-1: 62524371G/A
8-1: 62575029A/T
8-1: 62602761T/C
8-1: 62620503A/C
8-1: 62717741C/A
8-1: 62723787T/C
8-1: 62725343T/C
8-1: 62734137G/A
8-1: 63019227T/G
8-1: 63107417C/T
8-1: 63120270T/C
8-1: 63133829A/G
8-1: 63229160C/G
8-1: 63235594C/T
8-1: 63264989G/A
8-1: 63296130A/T
8-1: 63351199A/G
8-1: 63410783A/G
8-1: 63464132C/T
8-1: 63646923C/T
8-1: 63732113A/T
8-1: 63765536C/G
8-1: 63842369T/C
8-1: 63852545A/G
8-1: 63853242T/A
8-1: 63853395T/A
8-1: 63853637A/G
8-1: 63855194G/A
8-1: 63869263A/C
8-1: 63924611A/G
8-1: 64042207C/A
8-1: 64132164T/C
8-1: 64220356C/T
8-1: 64289426G/T
8-1: 64299573A/T
8-1: 64449835A/G
8-1: 64451227A/G
8-1: 64459923T/A
8-1: 64671972G/A
8-1: 64699628A/G
8-1: 64756209T/C
8-1: 64832242A/G
8-1: 64929843A/G
8-1: 64961795T/C
8-1: 65022209C/T
8-1: 65102216T/C
8-1: 65128063A/G
8-1: 65156616C/A
8-1: 65183967C/T
8-1: 65309550A/G
8-1: 65317137T/C
8-1: 65356566C/T
8-1: 65393496G/A
8-1: 65426317A/T
8-1: 65518396G/T
8-1: 65592594C/T
8-1: 65675231A/G
8-1: 65778276G/A
8-1: 65811543A/G
8-1: 65830483A/T
8-1: 65929519A/G
8-1: 66012298G/A
8-1: 66021609A/G
8-1: 66021614A/T
8-1: 66021645C/T
8-1: 66021652C/G
8-1: 66021678A/G
8-1: 66021739G/A
8-1: 66287323A/G
8-1: 66365333T/C
8-1: 66403550G/A
8-1: 66463897C/T
8-1: 66691356G/A
8-1: 66796333T/C
8-1: 66993602T/A
8-1: 67008885A/T
8-1: 67084978A/C
8-1: 67115532G/A
8-1: 67137450T/G
8-1: 67155265G/A
8-1: 67194777A/T
8-1: 67275795T/G
8-1: 67330478T/G
8-1: 67395638T/C
8-1: 67399743A/C
8-1: 67415645T/C
8-1: 67449123T/G
8-1: 67521336G/A
8-1: 67570625C/T
8-1: 67602456C/T
8-1: 67893364T/C
8-1: 67963035C/T
8-1: 68069350T/G
8-1: 68076823C/G
8-1: 68077635G/A
8-1: 68113943G/A
8-1: 68164537G/T
8-1: 68286286T/C
8-1: 68331269A/G
8-1: 68372795T/A
8-1: 68402825G/A
8-1: 68424964C/T
8-1: 68598401C/T
8-1: 68673695G/A
8-1: 68703326G/A
8-1: 68771089T/C
8-1: 68808979C/T
8-1: 68884322T/C
8-1: 69061141G/A
8-1: 69144817G/T
8-1: 69175663A/G
8-1: 69194103G/A
8-1: 69244270A/G
8-1: 69355916A/G
8-1: 69440242T/A
8-1: 69526145T/C
8-1: 69573070A/C
8-1: 69690022C/T
8-1: 69859501T/A
8-1: 70008224T/G
8-1: 70054839T/C
8-1: 70224487G/C
8-1: 70344469C/T
8-1: 70378191G/A
8-1: 70528615G/A
8-1: 70692350T/C
8-1: 70711374G/A
8-1: 70904575T/C
8-1: 70909415G/A
8-1: 70924695C/A
8-1: 70994485G/A
8-1: 71051800G/A
8-1: 71107071G/A
8-1: 71136989A/T
8-1: 71223284C/T
8-1: 71259069A/T
8-1: 71281762T/G
8-1: 71323921C/T
8-1: 71336927T/C
8-1: 71612999T/C
8-1: 71622065T/A
8-1: 71672678G/A
8-1: 71789781A/T
8-1: 71873226T/C
8-1: 71894999T/A
8-1: 71929322A/C
8-1: 72041671C/T
8-1: 72067589C/A
8-1: 72089421C/T
8-1: 72212910A/C
8-1: 72314264T/G
8-1: 72446975C/T
8-1: 72768029A/G
8-1: 72976271G/T
8-1: 73009030G/A
8-1: 73065763A/T
8-1: 73120180A/G
8-1: 73193775C/A
8-1: 73234690A/G
8-1: 73355452G/A
8-1: 73494226T/C
8-1: 73571595A/G
8-1: 73660028G/A
8-1: 73817605T/A
8-1: 73873235A/C
8-1: 73897003T/C
8-1: 73964696G/A
8-1: 74100887A/T
8-1: 74165767A/T
8-1: 74248253A/T
8-1: 74410668G/A
8-1: 74469183A/G
8-1: 74487514T/C
8-1: 74561062G/T
8-1: 74576727C/G
8-1: 74596077C/T
8-1: 74620167T/G
8-1: 74635646A/T
8-1: 74693479T/C
8-1: 74802280G/A
8-1: 74827012G/T
8-1: 74911471G/A
8-1: 74961248A/T
8-1: 75024134C/T
8-1: 75095906C/G
8-1: 75108188G/T
8-1: 75120311T/A
8-1: 75160634C/T
8-1: 75237510C/T
8-1: 75256105G/A
8-1: 75346435A/G
8-1: 75402070T/C
8-1: 75451016C/A
8-1: 75493798C/T
8-1: 75527051A/G
8-1: 75590367C/T
8-1: 75632978C/G
8-1: 75690383A/C
8-1: 75693930A/G
8-1: 75702977T/A
8-1: 75803447G/T
8-1: 75804464C/A
8-1: 75834232A/G
8-1: 75899920T/G
8-1: 75901322G/A
8-1: 75902580C/T
8-1: 75941302C/T
8-1: 75956462G/C
8-1: 76022610T/C
8-1: 76190830G/A
8-1: 76291332T/C
8-1: 76376398C/T
8-1: 76415916A/T
8-1: 76448341C/A
8-1: 76448818C/T
8-1: 76448861C/T
8-1: 76513404C/T
8-1: 76528930C/G
8-1: 76555252A/C
8-1: 76555458A/T
8-1: 76555533G/T
8-1: 76609656T/A
8-1: 76749115T/C
8-1: 76757488T/A
8-1: 76758638C/T
8-1: 76783482A/T
8-1: 76881716A/T
8-1: 76892881G/T
8-1: 76893985T/C
8-1: 76896822C/G
8-1: 76903670C/T
8-1: 76918828A/G
8-1: 77077683C/A
8-1: 77081866C/G
8-1: 77173494A/C
8-1: 77204783A/G
8-1: 77271422C/T
8-1: 77279775C/A
8-1: 77286439T/C
8-1: 77286441C/T
8-1: 77303113C/A
8-1: 77306471A/C
8-1: 77307695T/C
8-1: 77406927C/T
8-1: 77408340A/G
8-1: 77421201T/C
8-1: 77443141T/C
8-1: 77464767C/A
8-1: 77466542G/C
8-1: 77466569A/T
8-1: 77582960A/G
8-1: 77729545G/A
8-1: 77843388C/T
8-1: 77901538T/C
8-1: 77955911A/G
8-1: 78077256C/T
8-1: 78091597A/G
8-1: 78231854G/T
8-1: 78270039T/A
8-1: 78280835T/A
8-1: 78298353G/A
8-1: 78446009T/G
8-1: 78492140C/T
8-1: 78645158C/T
8-1: 78655166C/T
8-1: 78788500C/A
8-1: 78884952G/A
8-1: 78896501T/G
8-1: 78940016A/G
8-1: 78967037G/A
8-1: 79004206A/G
8-1: 79119102T/A
8-1: 79120228A/G
8-1: 79164502G/A
8-1: 79216418G/A
8-1: 79301801G/A
8-1: 79368316C/A
8-1: 79546770A/G
8-1: 79821205C/A
8-1: 79833401A/G
8-1: 79846751A/T
8-1: 79863416A/G
8-1: 79918089T/C
8-1: 79948413C/T
8-1: 80029923A/T
8-1: 80075403G/T
8-1: 80134850G/A
8-1: 80202300A/C
8-1: 80221210C/T
8-1: 80247527C/T
8-1: 80274721T/C
8-1: 80321010G/C
8-1: 80332037C/T
8-1: 80353519C/T
8-1: 80419722G/A
8-1: 80432996T/C
8-1: 80522828T/C
8-1: 80539934C/T
8-1: 80554781G/A
8-1: 80560490G/C
8-1: 80560754T/A
8-1: 80560854G/A
8-1: 80561134T/G
8-1: 80563701T/A
8-1: 80565452C/T
8-1: 80590754G/T
8-1: 80596765T/A
8-1: 80602258A/G
8-1: 80602778T/A
8-1: 80603226T/G
8-1: 80614496A/C
8-1: 80618537A/C
8-1: 80634289T/C
8-1: 80643127C/A
8-1: 80649362G/A
8-1: 80680956A/T
8-1: 80692839A/G
8-1: 80727377C/T
8-1: 80742657G/T
8-1: 80994262A/T
8-1: 81099850G/A
8-1: 81099864T/C
8-1: 81099876G/T
8-1: 81099878G/A
8-1: 81099903A/C
8-1: 81099905A/C
8-1: 81100015C/G
8-1: 81100024C/A
8-1: 81100394T/G
8-1: 81117035G/T
8-1: 81231438G/C
8-1: 81231828T/C
8-1: 81245693C/T
8-1: 81249353T/C
8-1: 81262578C/T
8-1: 81265566A/G
8-1: 81273405C/A
8-1: 81277425G/C
8-1: 81279103G/A
8-1: 81279349C/T
8-1: 81279966G/A
8-1: 81281367G/A
8-1: 81368818G/A
8-1: 81389714T/G
8-1: 81423614G/A
8-1: 81449669C/T
8-1: 81460350C/T
8-1: 81473135A/G
8-1: 81498874T/C
8-1: 81512389G/C
8-1: 81526892T/C
8-1: 81545596T/G
8-1: 81546922C/G
8-1: 81546941T/A
8-1: 81562518G/A
8-1: 81569617G/A
8-1: 81576046G/A
8-1: 81589224A/T
8-1: 81674192C/T
8-1: 81695078A/C
8-1: 81710447T/A
8-1: 81807459C/T
8-1: 81809018C/T
8-1: 81809042G/T
8-1: 81816286T/C
8-1: 81818422C/A
8-1: 81835507A/G
8-1: 81849586A/G
8-1: 81906787T/A
8-1: 81913347T/A
8-1: 81913395A/C
8-1: 81991862A/T
8-1: 82010601C/G
8-1: 82010791G/A
8-1: 82011105T/C
8-1: 82011587G/A
8-1: 82011876A/T
8-1: 82011898T/C
8-1: 82028284T/C
8-1: 82056551G/A
8-1: 82225988T/G
8-1: 82227776C/T
8-1: 82239863A/G
8-1: 82251136T/C
8-1: 82304234A/G
8-1: 82556991A/C
8-1: 82706332C/G
8-1: 82776464C/T
8-1: 82941009A/G
8-1: 83221659G/A
8-1: 83244430A/G
8-1: 83251151G/A
8-1: 83279155A/T
8-1: 83417354T/C
8-1: 83496186A/T
8-1: 83596940T/G
8-1: 83741242T/A
8-1: 83767272A/G
8-1: 83870856G/T
8-1: 84063089G/T
8-1: 84190298A/G
8-1: 84219614G/A
8-1: 84413644C/T
8-1: 84474972T/C
8-1: 84509895A/C
8-1: 84569264T/C
8-1: 84636538G/A
8-1: 84650298A/G
8-1: 84681482A/C
8-1: 84714331A/G
8-1: 84731944G/A
8-1: 84774201G/C
8-1: 84829204C/A
8-1: 84857324G/T
8-1: 84897405C/T
8-1: 84930723T/C
8-1: 84957880A/G
8-1: 85058424G/A
8-1: 85065121G/T
8-1: 85098088A/T
8-1: 85146933G/A
8-1: 85199502A/G
8-1: 85223823A/G
8-1: 85255591G/A
8-1: 85287124T/A
8-1: 85322396G/A
8-1: 85353912A/T
8-1: 85381065C/T
8-1: 85445415G/A
8-1: 85481498C/T
8-1: 85526746A/C
8-1: 85560732T/G
8-1: 85591097C/G
8-1: 85592242C/T
8-1: 85657010A/G
8-1: 85670800G/A
8-1: 85713162C/A
8-1: 85794886C/T
8-1: 85844812T/C
8-1: 85891844C/T
8-1: 85931470A/G
8-1: 85978743T/C
8-1: 86001976T/G
8-1: 86061917A/G
8-1: 86091565T/C
8-1: 86119013T/C
8-1: 86130697T/C
8-1: 86136078T/C
8-1: 86138018T/C
8-1: 86138084A/G
8-1: 86176227C/A
8-1: 86244190A/G
8-1: 86257992A/T
8-1: 86309498A/G
8-1: 86412821G/A
8-1: 86521714T/C
8-1: 86544456A/C
8-1: 86568210A/C
8-1: 86578848A/T
8-1: 86673534G/A
8-1: 86963445G/A
8-1: 86997282C/A
8-1: 87151895C/A
8-1: 87225543G/T
8-1: 87281563C/T
8-1: 87307160G/A
8-1: 87455682A/T
8-1: 87520895A/T
8-1: 87525408G/A
8-1: 87553194G/A
8-1: 87580933A/T
8-1: 87722518A/G
8-1: 87894109C/T
8-1: 87906748A/G
8-1: 87932462T/C
8-1: 87987857C/A
8-1: 87990731G/A
8-1: 87996941T/G
8-1: 87997085A/G
8-1: 87998124A/G
8-1: 88026995A/T
8-1: 88112191C/G
8-1: 88170740A/C
8-1: 88232153A/T
8-1: 88381646C/T
8-1: 88446594A/G
8-1: 88523118T/C
8-1: 88529919T/C
8-1: 88530009G/A
8-1: 88536022C/T
8-1: 88536387G/A
8-1: 88546581G/C
8-1: 88601096G/A
8-1: 88687176G/A
8-1: 88734113C/A
8-1: 88886397C/T
8-1: 89010006C/G
8-1: 89514497A/G
8-1: 89586601C/T
8-1: 89611883T/G
8-1: 89649476C/A
8-1: 89674091A/C
8-1: 89736462C/G
8-1: 89845374G/A
8-1: 89893288A/C
8-1: 89968437T/C
8-1: 90024104G/A
8-1: 90058236T/C
8-1: 90345085A/T
8-1: 90416795T/C
8-1: 90629540C/T
8-1: 90767120A/G
8-1: 91040684A/G
8-1: 91151538G/A
8-1: 91173878A/T
8-1: 91192354A/C
8-1: 91389190C/T
8-1: 91412184G/A
8-1: 91417301A/T
8-1: 91511773A/G
8-2: 24328T/C
8-2: 161099G/A
8-2: 311507A/C
8-2: 326711G/C
8-2: 326960T/A
8-2: 617889T/A
8-2: 930589A/G
8-2: 1010659A/T
8-2: 1219698C/A
8-2: 1342123G/A
8-2: 1556314T/A
8-2: 1668812T/C
8-2: 1766820G/T
8-2: 1883850T/A
8-2: 1933616T/C
8-2: 2112752G/A
8-2: 2200162G/A
8-2: 2200209T/C
8-2: 2254217C/T
8-2: 2313424G/A
8-2: 2402424T/G
8-2: 2415862G/A
8-2: 2429609A/G
8-2: 2501752A/T
8-2: 2522319G/C
8-2: 2547620T/C
8-2: 2561874T/A
8-2: 2712742C/A
8-2: 2787410G/A
8-2: 2810729G/A
8-2: 3019243A/G
8-2: 3071766C/A
8-2: 3150184T/A
8-2: 3191766T/C
8-2: 3269741C/T
8-2: 3353982C/G
8-2: 3379037A/T
8-2: 3468055A/T
8-2: 3521941G/A
8-2: 3554861G/A
8-2: 3644615G/T
8-2: 3698267T/C
8-2: 3715374G/A
8-2: 3816890T/A
8-2: 3827865G/C
8-2: 3878050G/A
8-2: 3948392C/T
8-2: 3980582A/G
8-2: 4010041C/T
8-2: 4033743C/T
8-2: 4082935G/A
8-2: 4083221C/T
8-2: 4083576C/T
8-2: 4099165C/T
8-2: 4112665A/C
8-2: 4118627A/T
8-2: 4126825G/A
8-2: 4131179T/A
8-2: 4171355A/T
8-2: 4223194G/C
8-2: 4259116C/A
8-2: 4290825A/T
8-2: 4330282G/A
8-2: 4350493T/C
8-2: 4430416T/C
8-2: 4488125G/T
8-2: 4525379C/T
8-2: 4555107T/C
8-2: 4704366T/C
8-2: 4720758G/T
8-2: 4874596G/T
8-2: 4917114C/T
8-2: 4968552T/G
8-2: 5035772C/T
8-2: 5069776A/C
8-2: 5073432A/G
8-2: 5075182C/T
8-2: 5083834C/T
8-2: 5203028C/T
8-2: 5280956T/A
8-2: 5327370G/A
8-2: 5542077T/C
8-2: 5625997T/C
8-2: 5647968G/T
8-2: 5723951G/T
8-2: 5743881G/A
8-2: 5743983G/T
8-2: 5755088C/T
8-2: 5802249G/A
8-2: 5827636C/T
8-2: 5978716G/A
8-2: 6074529G/A
8-2: 6113696C/.
8-2: 6116845A/G
8-2: 6196065A/C
8-2: 6200241T/A
8-2: 6219097G/A
8-2: 6318955C/T
8-2: 6397224C/T
8-2: 6412228C/T
8-2: 6414190A/T
8-2: 6422767C/A
8-2: 6449951T/A
8-2: 6450277A/G
8-2: 6452533C/T
8-2: 6514105G/T
8-2: 6514493C/T
8-2: 6517072C/T
8-2: 6658738C/T
8-2: 6712209T/C
8-2: 7071033A/T
8-2: 7264767C/T
8-2: 7270354T/C
8-2: 7270728A/C
8-2: 7270915G/T
8-2: 7271066C/A
8-2: 7271393T/A
8-2: 7275555C/T
8-2: 7291220T/C
8-2: 7339532C/G
8-2: 7349963T/A
8-2: 7350099T/A
8-2: 7382707C/G
8-2: 7382834T/C
8-2: 7408342T/A
8-2: 7465601A/T
8-2: 7497266T/G
8-2: 7534810G/T
8-2: 7578450T/C
8-2: 7630268T/C
8-2: 7631286C/A
8-2: 7632864C/G
8-2: 7633020G/T
8-2: 7659197C/A
8-2: 7690992T/C
8-2: 7705375A/G
8-2: 7804397T/G
8-2: 7860533T/G
8-2: 7911322A/C
8-2: 7960176C/A
8-2: 7976321T/G
8-2: 8018714G/A
8-2: 8029593T/G
8-2: 8072417A/G
8-2: 8073981A/G
8-2: 8098492G/C
8-2: 8161810C/T
8-2: 8199304G/T
8-2: 8225954A/G
8-2: 8303394G/A
8-2: 8335205C/T
8-2: 8368323A/T
8-2: 8500726A/T
8-2: 8502453C/T
8-2: 8615135A/C
8-2: 8676757G/A
8-2: 8767033G/A
8-2: 8924320C/G
8-2: 9016111G/C
8-2: 9029311C/A
8-2: 9102729C/T
8-2: 9178450A/G
8-2: 9229265A/G
8-2: 9235930A/T
8-2: 9257076C/T
8-2: 9260203T/C
8-2: 9261665C/T
8-2: 9458637T/G
8-2: 9471523A/T
8-2: 9494546T/A
8-2: 9515459G/A
8-2: 9575989G/A
8-2: 9646328G/C
8-2: 9706996T/C
8-2: 9732698G/A
8-2: 9838675A/G
8-2: 9887758C/T
8-2: 9906263G/A
8-2: 9941787T/C
8-2: 9943321A/G
8-2: 9944769G/A
8-2: 9958495A/T
8-2: 10192225C/G
8-2: 10243487T/C
8-2: 10281851C/A
8-2: 10298751A/G
8-2: 10322804C/A
8-2: 10349790C/T
8-2: 10350203G/T
8-2: 10350619T/C
8-2: 10382187C/T
8-2: 10406742T/C
8-2: 10430688C/T
8-2: 10445518G/A
8-2: 10541328T/A
8-2: 10580184C/T
8-2: 10665395C/A
8-2: 10665878A/G
8-2: 10679466A/G
8-2: 10683307T/A
8-2: 10705544C/G
8-2: 10734582T/G
8-2: 10747783G/T
8-2: 10764664T/G
8-2: 10797349A/G
8-2: 10823320G/A
8-2: 10851064T/C
8-2: 10861827A/G
8-2: 10888527C/A
8-2: 10941696G/T
8-2: 10970206A/C
8-2: 10985580T/C
8-2: 11002752T/C
8-2: 11052232A/T
8-2: 11052356T/A
8-2: 11052653G/A
8-2: 11052735G/A
8-2: 11052771G/T
8-2: 11059190G/A
8-2: 11074580T/C
8-2: 11097889T/A
8-2: 11099795C/A
8-2: 11099904C/T
8-2: 11100009C/A
8-2: 11101314G/C
8-2: 11115748C/T
8-2: 11122599G/A
8-2: 11170386C/A
8-2: 11226188T/C
8-2: 11270877G/T
8-2: 11296771G/A
8-2: 11336137G/T
8-2: 11509091T/C
8-2: 11554307C/A
8-2: 11554327A/G
8-2: 11599787A/G
8-2: 11616600C/T
8-2: 11665687T/C
8-2: 11738558C/T
8-2: 11768567C/T
8-2: 11794171T/A
8-2: 11896687A/T
8-2: 11974838T/C
8-2: 12003598A/C
8-2: 12064017A/T
8-2: 12116251C/A
8-2: 12175307C/G
8-2: 12286713G/T
8-2: 12287927G/C
8-2: 12342410A/C
8-2: 12376945C/T
8-2: 12420177C/A
8-2: 12436203C/T
8-2: 12478276A/G
8-2: 12500781T/C
8-2: 12530782A/T
8-2: 12553754T/A
8-2: 12553877A/G
8-2: 12636892G/T
8-2: 12740111A/G
8-2: 12785541T/C
8-2: 12820711G/A
8-2: 12874902C/A
8-2: 12915873C/A
8-2: 12983548A/G
8-2: 13008762T/G
8-2: 13036988G/T
8-2: 13100277C/T
8-2: 13130575T/G
8-2: 13132010C/T
8-2: 13202222C/G
8-2: 13301045T/C
8-2: 13362262G/A
8-2: 13395815C/T
8-2: 13432071T/C
8-2: 13511800C/A
8-2: 13565407T/A
8-2: 13644876C/T
8-2: 13645598T/C
8-2: 13645797C/T
8-2: 13646012C/T
8-2: 13646302C/T
8-2: 13646434G/A
8-2: 13646908C/T
8-2: 13684882T/C
8-2: 13732123G/A
8-2: 13838066C/G
8-2: 13864548C/T
8-2: 13896088A/G
8-2: 13927324T/C
8-2: 14009698T/C
8-2: 14126198T/C
8-2: 14141556T/C
8-2: 14171384T/A
8-2: 14217429A/G
8-2: 14272681C/T
8-2: 14308362G/A
8-2: 14526035G/T
8-2: 14575874C/A
8-2: 14636614A/G
8-2: 14675435A/T
8-2: 14739247A/G
8-2: 14757403A/T
8-2: 14799342T/A
8-2: 14890148A/G
8-2: 14981241C/T
8-2: 15064683A/T
8-2: 15219732T/A
8-2: 15270022G/A
8-2: 15314981A/G
8-2: 15337555T/C
8-2: 15356430G/T
8-2: 15385462A/G
8-2: 15416706A/G
8-2: 15506532T/G
8-2: 15533251A/G
8-2: 15558970G/T
8-2: 15573412T/G
8-2: 15595078C/T
8-2: 15595185T/A
8-2: 15595304A/G
8-2: 15595313G/T
8-2: 15595610A/C
8-2: 15595760G/A
8-2: 15617614G/T
8-2: 15699074G/A
8-2: 15729865A/C
8-2: 15806123T/C
8-2: 15938545A/G
8-2: 15950796A/G
8-2: 16013961A/G
8-2: 16074588C/T
8-2: 16100358T/A
8-2: 16117670A/G
8-2: 16142914T/G
8-2: 16171660G/A
8-2: 16200053T/A
8-2: 16297049C/T
8-2: 16331831C/A
8-2: 16339320A/G
8-2: 16377089A/G
8-2: 16436171G/C
8-2: 16517903A/G
8-2: 16550250A/G
8-2: 16611615G/A
8-2: 16726211T/C
8-2: 16771873T/C
8-2: 16867694C/T
8-2: 16898729T/C
8-2: 16968095G/C
8-2: 16997331G/A
8-2: 17028892G/T
8-2: 17068776A/G
8-2: 17103097A/T
8-2: 17161146T/G
8-2: 17161452G/A
8-2: 17198941C/G
8-2: 17227418C/T
8-2: 17346824C/T
8-2: 17379137G/T
8-2: 17471868A/T
8-2: 17562871G/A
8-2: 17584230G/A
8-2: 17630345T/A
8-2: 17654354G/A
8-2: 17689105G/A
8-2: 17713989T/C
8-2: 17745491G/A
8-2: 17760019T/G
8-2: 17841625C/T
8-2: 17953996G/A
8-2: 18061089T/A
8-2: 18120261C/T
8-2: 18183990T/C
8-2: 18309588C/G
8-2: 18349285C/T
8-2: 18450234A/T
8-2: 18625111T/C
8-2: 18657938T/C
8-2: 18663909T/C
8-2: 18664062G/A
8-2: 18664886G/A
8-2: 18677415A/G
8-2: 18703470G/T
8-2: 18814923T/C
8-2: 18816016C/T
8-2: 18838778C/T
8-2: 18841612T/A
8-2: 18849446C/A
8-2: 18879023C/T
8-2: 18881006A/C
8-2: 18893106C/A
8-2: 18906604T/G
8-2: 18918000G/A
8-2: 18971550A/C
8-2: 18973523A/G
8-2: 19008321C/A
8-2: 19046816G/A
8-2: 19089832C/T
8-2: 19093406G/A
8-2: 19105305A/T
8-2: 19152149G/C
8-2: 19199028C/T
8-2: 19326143A/T
8-2: 19344829T/C
8-2: 19350674C/T
8-2: 19350780G/T
8-2: 19359952A/C
8-2: 19374513G/A
8-2: 19390835A/G
8-2: 19403998A/G
8-2: 19423723T/G
8-2: 19464375G/A
8-2: 19516160A/T
8-2: 19582017G/A
8-2: 19622574C/T
8-2: 19633291A/G
8-2: 19760138C/T
8-2: 19811049C/A
8-2: 19910191T/G
8-2: 19947384A/G
8-2: 19975237G/A
8-2: 20003917T/C
8-2: 20008063T/.
8-2: 20008539T/C
8-2: 20066413A/G
8-2: 20175990G/A
8-2: 20192767T/G
8-2: 20217861A/G
8-2: 20263151G/A
8-2: 20264019A/G
8-2: 20264047T/C
8-2: 20275608T/C
8-2: 20288522C/G
8-2: 20289720T/C
8-2: 20374176G/A
8-2: 20422646A/T
8-2: 20429841T/C
8-2: 20430260T/C
8-2: 20509699T/C
8-2: 20626400T/C
8-2: 20676095C/T
8-2: 20743077A/T
8-2: 20825154A/T
8-2: 20867493C/G
8-2: 20915091T/G
8-2: 20990912A/G
8-2: 21010103C/T
8-2: 21059649T/C
8-2: 21059860C/T
8-2: 21059978C/T
8-2: 21060108G/T
8-2: 21081055C/T
8-2: 21113219A/G
8-2: 21139024C/T
8-2: 21197834A/G
8-2: 21209228T/C
8-2: 21232283C/T
8-2: 21279913A/T
8-2: 21345054G/A
8-2: 21382944T/A
8-2: 21405540C/T
8-2: 21453837C/A
8-2: 21486782T/A
8-2: 21530650A/C
8-2: 21595052C/T
8-2: 21614091A/G
8-2: 21635210C/T
8-2: 21677603T/C
8-2: 21704566C/T
8-2: 21732556G/A
8-2: 21759106A/G
8-2: 21843550G/A
8-2: 21928012C/T
8-2: 21967538C/T
8-2: 21995589C/T
8-2: 22021115T/C
8-2: 22066366A/G
8-2: 22116074C/T
8-2: 22156359C/G
8-2: 22191069T/G
8-2: 22253298T/A
8-2: 22274122C/T
8-2: 22317277G/C
8-2: 22334818G/A
8-2: 22363730C/T
8-2: 22381252T/C
8-2: 22413692G/A
8-2: 22426834A/C
8-2: 22493619T/C
8-2: 22523897A/G
8-2: 22536698C/T
8-2: 22660687T/A
8-2: 22675711A/G
8-2: 22709910A/G
8-2: 22723566A/G
8-2: 22751859T/C
8-2: 22804857T/C
8-2: 22822590A/G
8-2: 22838543C/T
8-2: 22861581G/T
8-2: 22953594A/T
8-2: 22955613A/G
8-2: 22971232A/C
8-2: 22987311C/T
8-2: 23013493T/G
8-2: 23026188T/A
8-2: 23046308T/C
8-2: 23058048G/A
8-2: 23099293A/T
8-2: 23240573T/A
8-2: 23268195A/G
8-2: 23328571T/A
8-2: 23350512G/A
8-2: 23599935T/C
8-2: 23630487C/T
8-2: 23644684G/T
8-2: 23679204G/A
8-2: 23684670C/T
8-2: 23697943A/G
8-2: 23701659A/G
8-2: 23758636C/T
8-2: 23767359C/T
8-2: 23767405G/A
8-2: 23767566G/A
8-2: 23767764T/C
8-2: 23768095G/A
8-2: 23768110G/T
8-2: 23768691C/T
8-2: 23781163A/G
8-2: 23792924C/A
8-2: 23796546G/A
8-2: 23799207A/G
8-2: 23799415C/T
8-2: 23799461T/C
8-2: 23822798T/A
8-2: 23996057C/A
8-2: 24017897A/G
8-2: 24062446T/G
8-2: 24078214C/T
8-2: 24119210G/C
8-2: 24242000A/C
8-2: 24270971A/T
8-2: 24316945C/T
8-2: 24418049A/G
8-2: 24474464G/A
8-2: 24487169G/A
8-2: 24519632A/C
8-2: 24559799T/G
8-2: 24601380C/T
8-2: 24606534T/C
8-2: 24660079C/T
8-2: 24694120C/T
8-2: 24705559C/A
8-2: 24727728A/C
8-2: 24734976C/T
8-2: 24808749T/G
8-2: 24885583A/G
8-2: 24950898T/C
8-2: 25010600T/C
8-2: 25071271G/T
8-2: 25150533G/T
8-2: 25167560A/T
8-2: 25178256G/A
8-2: 25192642T/G
8-2: 25231503T/C
8-2: 25258454C/A
8-2: 25274706G/A
8-2: 25288572T/C
8-2: 25297988G/A
8-2: 25308471C/G
8-2: 25309886G/T
8-2: 25315035T/C
8-2: 25327141G/T
8-2: 25340410A/G
8-2: 25373274A/G
8-2: 25417529G/A
8-2: 25430997G/A
8-2: 25443424C/T
8-2: 25487259C/T
8-2: 25494646A/G
8-2: 25542178C/G
8-2: 25567814A/T
8-2: 25613069C/T
8-2: 25632797A/G
8-2: 25695028A/T
8-2: 25718514C/T
8-2: 25731608T/A
8-2: 25790038T/C
8-2: 25808811T/C
8-2: 25829727G/A
8-2: 25859887G/C
8-2: 25875752A/G
8-2: 25878162T/A
8-2: 25897304T/A
8-2: 25897305T/C
8-2: 25897320A/C
8-2: 25897428C/T
8-2: 25904031C/T
8-2: 25989976G/T
8-2: 26023039T/C
8-2: 26064604C/T
8-2: 26135847T/A
8-2: 26331951T/C
8-2: 26371520T/C
8-2: 26414911C/T
8-2: 26445365C/T
8-2: 26458286C/T
8-2: 26490906C/T
8-2: 26524912A/G
8-2: 26526688G/A
8-2: 26580372T/A
8-2: 26595854G/A
8-2: 26653474A/C
8-2: 26661917T/G
8-2: 26662037A/T
8-2: 26662752G/T
8-2: 26707100G/A
8-2: 26724008T/C
8-2: 26727073A/C
8-2: 26795917C/A
8-2: 26842960A/G
8-2: 26843550T/C
8-2: 26848076C/T
8-2: 26848200C/A
8-2: 26848261A/G
8-2: 26855952C/T
8-2: 26866440C/T
8-2: 26894727G/A
8-2: 26909638A/T
8-2: 26925366T/C
8-2: 26937304T/G
8-2: 27013320C/T
8-2: 27078262A/G
8-2: 27131487T/C
8-2: 27142930C/T
8-2: 27154290G/T
8-2: 27244778C/G
8-2: 27328845T/C
8-2: 27386039C/A
8-2: 27434864G/T
8-2: 27461154T/G
8-2: 27496866A/G
8-2: 27546622T/A
8-2: 27558840A/T
8-2: 27560340A/G
8-2: 27604239C/T
8-2: 27659012T/A
8-2: 27660551T/C
8-2: 27699354G/T
8-2: 27725556A/G
8-2: 27782609A/G
8-2: 27813449C/T
8-2: 27908061T/C
8-2: 27974262G/T
8-2: 28024658T/C
8-2: 28062663T/G
8-2: 28101256T/C
8-2: 28163889T/A
8-2: 28187297T/C
8-2: 28222634A/G
8-2: 28241386C/A
8-2: 28377764T/C
8-2: 28507666C/T
8-2: 28520586G/A
8-2: 28609413A/G
8-2: 28690549C/G
8-2: 28769193G/T
8-2: 28791095T/A
8-2: 28825999C/A
8-2: 28990450G/C
8-2: 28991512C/T
8-2: 28993101T/G
8-2: 29005358C/T
8-2: 29075172C/T
8-2: 29155958G/T
8-2: 29177020T/A
8-2: 29381441C/G
8-2: 29465328A/G
8-2: 29472102G/A
8-2: 29493542G/C
8-2: 29532510G/A
8-2: 29562975T/G
8-2: 29585637C/A
8-2: 29589400T/C
8-2: 29605631G/A
8-2: 29620118A/G
8-2: 29620621G/A
8-2: 29621415G/A
8-2: 29631719A/G
8-2: 29650103C/G
8-2: 29682102C/T
8-2: 29768746A/G
8-2: 29797305T/C
8-2: 29843919G/T
8-2: 29905407A/T
8-2: 29990467A/T
8-2: 30129201T/C
8-2: 30134718G/A
8-2: 30147757C/T
8-2: 30166993T/A
8-2: 30223620G/A
8-2: 30249307T/A
8-2: 30304717C/A
8-2: 30362210A/G
8-2: 30401875C/T
8-2: 30445798A/T
8-2: 30503039C/A
8-2: 30609549A/G
8-2: 30609756C/T
8-2: 30609866G/T
8-2: 30609984C/G
8-2: 30610080T/G
8-2: 30610092G/T
8-2: 30621071T/C
8-2: 30627358T/A
8-2: 30634304G/A
8-2: 30658887T/C
8-2: 30672409G/T
8-2: 30714378A/T
8-2: 30761015C/T
8-2: 30803671T/A
8-2: 30834420T/C
8-2: 30890195T/G
8-2: 31023643C/T
8-2: 31184440G/C
8-2: 31212108A/T
8-2: 31244951G/T
8-2: 31293211A/C
8-2: 31324252T/A
8-2: 31350755C/T
8-2: 31421360C/T
8-2: 31555213C/T
8-2: 31595108C/T
8-2: 31622454T/C
8-2: 31634424T/C
8-2: 31634953T/C
8-2: 31637493A/G
8-2: 31662667G/A
8-2: 31706269A/T
8-2: 31730951G/A
8-2: 31760750C/T
8-2: 31807987C/G
8-2: 31849094G/A
8-2: 31907999A/C
8-2: 31959939A/G
8-2: 31970891T/A
8-2: 31981079C/A
8-2: 31981861G/A
8-2: 31981910T/C
8-2: 31982020T/A
8-2: 31983153G/A
8-2: 31983258G/A
8-2: 31991465T/G
8-2: 31992133A/T
8-2: 32057597C/T
8-2: 32083065T/A
8-2: 32083270G/A
8-2: 32086125C/T
8-2: 32109589T/A
8-2: 32164231A/T
8-2: 32226774C/T
8-2: 32227708C/T
8-2: 32304890T/A
8-2: 32399770A/C
8-2: 32442201C/G
8-2: 32455898G/A
8-2: 32469174T/G
8-2: 32661469T/G
8-2: 32681030T/C
8-2: 32692387C/T
8-2: 32720946G/A
8-2: 32752899T/C
8-2: 32786379C/A
8-2: 32854610C/T
8-2: 32903862T/C
8-2: 33065224C/G
8-2: 33141420A/C
8-2: 33172451A/G
8-2: 33172525A/G
8-2: 33173104A/T
8-2: 33221051T/G
8-2: 33320261G/T
8-2: 33337949G/T
8-2: 33405659G/A
8-2: 33553925A/C
8-2: 33582141T/C
8-2: 33642992A/T
8-2: 33788787G/A
8-2: 33864117G/A
8-2: 33925787G/A
8-2: 33996256A/G
8-2: 34022523T/C
8-2: 34141742C/A
8-2: 34174927A/G
8-2: 34343649A/C
8-2: 34357757G/T
8-2: 34357858A/G
8-2: 34382303G/C
8-2: 34406708T/C
8-2: 34406723A/G
8-2: 34406801T/G
8-2: 34406869G/A
8-2: 34415099T/C
8-2: 34518984G/A
8-2: 34550188G/C
8-2: 34566081C/T
8-2: 34586115T/A
8-2: 34611666G/A
8-2: 34617177T/C
8-2: 34706522T/C
8-2: 34749962C/T
8-2: 34750036T/C
8-2: 34760633C/A
8-2: 34763748G/T
8-2: 34764423C/T
8-2: 34764721A/G
8-2: 34764744C/T
8-2: 34764768T/C
8-2: 34925126A/G
8-2: 35077426C/T
8-2: 35123158A/G
8-2: 35124457G/A
8-2: 35124630C/T
8-2: 35156439G/A
8-2: 35157174G/A
8-2: 35189714G/A
8-2: 35245172G/A
8-2: 35353676C/A
8-2: 35491991C/A
8-2: 35515171T/A
8-2: 35525443T/C
8-2: 35536405G/A
8-2: 35565691T/C
8-2: 35592178C/T
8-2: 35622966A/G
8-2: 35681312C/T
8-2: 35695095C/T
8-2: 35697245C/T
8-2: 35742634C/T
8-2: 35783428T/A
8-2: 35855450T/C
8-2: 35912273A/T
8-2: 36054382G/A
8-2: 36248808G/A
8-2: 36296544T/G
8-2: 36317320C/T
8-2: 36323697A/T
8-2: 36419614A/G
8-2: 36461279G/A
8-2: 36533366T/A
8-2: 36732392C/G
8-2: 36848998G/A
8-2: 36890809G/A
8-2: 36905753G/A
8-2: 36908319G/A
8-2: 36908387T/A
8-2: 36908516C/T
8-2: 36908758T/G
8-2: 36909440A/G
8-2: 36909509A/T
8-2: 36948679T/A
8-2: 37161254G/A
8-2: 37260433G/A
8-2: 37478316G/A
8-2: 37509657T/A
8-2: 37512197A/T
8-2: 37519957T/C
8-2: 37579273T/A
8-2: 37653771A/G
8-2: 37673962C/T
8-2: 37691755T/A
8-2: 37707758T/C
8-2: 37707910T/C
8-2: 37737565T/C
8-2: 37822810C/T
8-2: 37830358C/A
8-2: 37867856T/G
8-2: 37882487T/C
8-2: 37934587C/T
8-2: 37960068T/G
8-2: 37995291G/C
8-2: 38035747G/T
8-2: 38047872C/T
8-2: 38071943G/A
8-2: 38089335C/T
8-2: 38097229C/T
8-2: 38103702G/A
8-2: 38164228C/A
8-2: 38183898C/T
8-2: 38189433A/T
8-2: 38189477G/A
8-2: 38206326G/T
8-2: 38208272G/A
8-2: 38243271C/A
8-2: 38253326A/G
8-2: 38309394G/A
8-2: 38375817C/T
8-2: 38462136A/G
8-2: 38464435G/A
8-2: 38549836T/A
8-2: 38561248C/T
8-2: 38590236C/A
8-2: 38605243A/G
8-2: 38621443G/A
8-2: 38622367G/A
8-2: 38684548G/A
8-2: 38728467T/G
8-2: 38764407T/G
8-2: 38778078C/A
8-2: 38821955T/C
8-2: 38853964T/G
8-2: 38899887A/T
8-2: 38935711C/T
8-2: 38977603T/A
8-2: 39039994G/A
8-2: 39051470G/C
8-2: 39055933A/T
8-2: 39058897T/C
8-2: 39064488G/A
8-2: 39077049C/T
8-2: 39117024G/T
8-2: 39199033T/A
8-2: 39200252T/A
8-2: 39229121A/T
8-2: 39284542T/A
8-2: 39285111A/T
8-2: 39285262A/C
8-2: 39335028T/G
8-2: 39353984C/T
8-2: 39387506A/G
8-2: 39392521T/C
8-2: 39409610G/A
8-2: 39444802G/A
8-2: 39487740T/C
8-2: 39582244C/T
8-2: 39626345C/A
8-2: 39705284T/C
8-2: 39761408C/T
8-2: 39761433C/A
8-2: 39773429A/G
8-2: 39778346G/A
8-2: 39808763G/T
8-2: 39830200T/C
8-2: 39858610C/T
8-2: 39873926C/A
8-2: 39977936T/C
8-2: 40067508C/A
8-2: 40072062A/G
8-2: 40072394T/A
8-2: 40073018A/T
8-2: 40083236T/G
8-2: 40101403T/C
8-2: 40210450T/A
8-2: 40213082C/A
8-2: 40226741T/A
8-2: 40243967C/T
8-2: 40345201A/G
8-2: 40396869T/A
8-2: 40442235C/T
8-2: 40463143G/A
8-2: 40521415A/T
8-2: 40558241G/A
8-2: 40618966A/T
8-2: 40669272G/A
8-2: 40669302C/T
8-2: 40695712T/C
8-2: 40697576C/T
8-2: 40697684A/G
8-2: 40720676C/A
8-2: 40720782C/T
8-2: 40753316C/T
8-2: 40793867T/C
8-2: 40796914T/C
8-2: 40826135A/G
8-2: 40991534A/G
8-2: 41066585A/G
8-2: 41384619A/G
8-2: 41385035C/T
8-2: 41385199G/C
8-2: 41385209G/A
8-2: 41412180T/A
8-2: 41412606C/T
8-2: 41412687G/A
8-2: 41425911A/G
8-2: 41448325A/T
8-2: 41448812C/T
8-2: 41448854T/A
8-2: 41469410C/A
8-2: 41512303A/T
8-2: 41529270A/G
8-2: 41615622T/A
8-2: 41646291T/G
8-2: 41656992C/T
8-2: 41678286C/T
8-2: 41689667G/T
8-2: 41799387A/G
8-2: 41810650A/G
8-2: 41811618G/A
8-2: 41830378C/T
8-2: 41847402C/T
8-2: 41858724C/A
8-2: 41882603T/G
8-2: 41893833G/T
8-2: 41945347G/A
8-2: 41955402T/G
8-2: 41994798G/T
8-2: 41996789C/A
8-2: 42007298G/A
8-2: 42247075C/T
8-2: 42386014A/G
8-2: 42460736G/A
8-2: 42477324T/G
8-2: 42501984T/G
8-2: 42501991G/T
8-2: 42508604A/T
8-2: 42508822C/T
8-2: 42509101A/G
8-2: 42509916A/G
8-2: 42510355G/A
8-2: 42512747T/C
8-2: 42519441G/A
8-2: 42523271G/A
8-2: 42558786A/T
8-2: 42657094A/C
8-2: 42684193G/A
8-2: 42699345G/T
8-2: 42728569T/C
8-2: 42767706C/A
8-2: 42768515T/A
8-2: 42800809G/A
8-2: 42865596C/T
8-2: 42871675G/A
8-2: 42891769T/G
8-2: 42938554T/C
8-2: 42949332C/T
8-2: 42959362A/G
8-2: 43049590C/T
8-2: 43078756G/T
8-2: 43108312T/C
8-2: 43349562C/G
8-2: 43472855T/G
8-2: 43507205A/T
8-2: 43576977G/T
8-2: 43659359C/T
8-2: 43661080G/A
8-2: 43719447C/A
8-2: 43748685A/C
8-2: 43749951A/T
8-2: 43789447C/T
8-2: 43968650C/G
8-2: 43972424A/G
8-2: 44085910A/G
8-2: 44120154T/C
8-2: 44132529T/A
8-2: 44174855A/T
8-2: 44205433T/C
8-2: 44310007G/T
8-2: 44389052T/A
8-2: 44465536A/T
8-2: 44477423G/A
8-2: 44512840A/T
8-2: 44514308G/T
8-2: 44531785G/C
8-2: 44594798A/T
8-2: 44598472C/G
8-2: 44598755G/T
8-2: 44599103T/C
8-2: 44607649C/A
8-2: 44680481T/C
8-2: 44705533A/T
8-2: 44747836C/G
8-2: 44782554G/A
8-2: 44869269C/T
8-2: 44918622C/T
8-2: 44995373T/C
8-2: 45045155G/A
8-2: 45096227C/T
8-2: 45139774C/T
8-2: 45151028A/C
8-2: 45163876T/C
8-2: 45163917C/T
8-2: 45163919G/A
8-2: 45163923A/C
8-2: 45163942G/T
8-2: 45163960G/A
8-2: 45164225C/A
8-2: 45164238G/A
8-2: 45164541C/T
8-2: 45164558T/C
8-2: 45164624A/C
8-2: 45164626G/A
8-2: 45364200T/C
8-2: 45472737A/T
8-2: 45514944G/T
8-2: 45597255G/A
8-2: 45754611G/A
8-2: 45778316A/G
8-2: 45866350A/G
8-2: 45910420A/G
8-2: 45924959C/T
8-2: 45946753C/T
8-2: 45947737T/A
8-2: 45978075G/T
8-2: 46033291A/C
8-2: 46092459G/A
8-2: 46121208T/C
8-2: 46194433G/A
8-2: 46337039G/A
8-2: 46391532G/T
8-2: 46491766A/G
8-2: 46518277A/C
8-2: 46531142C/T
8-2: 46568567T/C
8-2: 46614345A/T
8-2: 46619061A/G
8-2: 46678009G/A
8-2: 46783417T/A
8-2: 46794436A/G
8-2: 46840732A/T
8-2: 46867441T/A
8-2: 46931897T/C
8-2: 46933001G/A
8-2: 46936523A/G
8-2: 46961950A/G
8-2: 46962920T/C
8-2: 46962960T/A
8-2: 46971203C/T
8-2: 46989775C/G
8-2: 47028040G/T
8-2: 47072861A/T
8-2: 47086649A/G
8-2: 47115930G/A
8-2: 47121952G/A
8-2: 47127163T/C
8-2: 47226226G/T
8-2: 47236480T/A
8-2: 47261457C/G
8-2: 47272333A/G
8-2: 47293600A/C
8-2: 47296763A/T
8-2: 47305269G/A
8-2: 47308972T/C
8-2: 47332342G/A
8-2: 47339068G/C
8-2: 47339071G/A
8-2: 47366122G/A
8-2: 47366284G/T
8-2: 47400774C/G
8-2: 47426950T/A
8-2: 47452266G/A
8-2: 47462920C/A
8-2: 47480917A/G
8-2: 47485545G/T
8-2: 47485592G/T
8-2: 47495504T/C
8-2: 47522597G/A
8-2: 47551531A/G
8-2: 47551746T/A
8-2: 47554471T/C
8-2: 47555998T/C
8-2: 47561080A/G
8-2: 47561680G/A
8-2: 47604658A/G
8-2: 47691207T/A
8-2: 47705935C/A
8-2: 47765325G/A
8-2: 47863159A/G
8-2: 47872810T/A
8-2: 47872952C/G
8-2: 47873309A/G
8-2: 47873683A/G
8-2: 47873880T/A
8-2: 47875170A/T
8-2: 47903395C/T
8-2: 47923627C/T
8-2: 47936415G/A
8-2: 47955142T/A
8-2: 47957659A/G
8-2: 48003357C/T
8-2: 48065785A/G
8-2: 48087012T/C
8-2: 48121244C/A
8-2: 48140939G/T
8-2: 48148649A/C
8-2: 48148763C/T
8-2: 48148870T/C
8-2: 48150488T/C
8-2: 48151488A/G
8-2: 48152088G/T
8-2: 48182841A/T
8-2: 48202637A/G
8-2: 48202823C/G
8-2: 48211030A/G
8-2: 48240932C/T
8-2: 48286890T/A
8-2: 48314791C/A
8-2: 48332848T/G
8-2: 48345199G/A
8-2: 48362625G/T
8-2: 48367499G/A
8-2: 48373363T/A
8-2: 48386680G/C
8-2: 48457216T/A
8-2: 48481634T/C
8-2: 48487409T/C
8-2: 48487478A/G
8-2: 48492499A/G
8-2: 48494226A/T
8-2: 48514303A/G
8-2: 48557787A/G
8-2: 48601718T/C
8-2: 48629164T/C
8-2: 48657427T/C
8-2: 48690925T/C
8-2: 48727702A/C
8-2: 48734988A/G
8-2: 48735130G/A
8-2: 48768174C/A
8-2: 48768284T/A
8-2: 48792195A/T
8-2: 48805577C/A
8-2: 48811334T/C
8-2: 48815265T/C
8-2: 48815399G/A
8-2: 48815502T/C
8-2: 48815616A/T
8-2: 48815707A/G
8-2: 48815808G/A
8-2: 48816578C/T
8-2: 48816741G/T
8-2: 48829052A/T
8-2: 48841684T/G
8-2: 48860660C/T
8-2: 48869847T/C
8-2: 48895575G/A
8-2: 48907795G/T
8-2: 48982852G/A
8-2: 49012212C/A
8-2: 49032509T/A
8-2: 49047056T/C
8-2: 49064951G/T
8-2: 49085962T/G
8-2: 49086274A/T
8-2: 49088050A/C
8-2: 49103513G/C
8-2: 49107651T/G
8-2: 49113665T/C
8-2: 49124428A/T
8-2: 49124948G/A
8-2: 49135437A/T
8-2: 49161030T/A
8-2: 49203818C/A
8-2: 49214068C/T
8-2: 49247585C/G
8-2: 49257832G/A
8-2: 49301920T/G
8-2: 49351037T/A
8-2: 49389895A/T
8-2: 49406366T/G
8-2: 49430705T/G
8-2: 49439092A/T
8-2: 49455233G/A
8-2: 49493405G/T
8-2: 49533551T/C
8-2: 49563275T/C
8-2: 49563552C/A
8-2: 49563677T/A
8-2: 49565351C/T
8-2: 49565461C/A
8-2: 49590429A/G
8-2: 49602447T/C
8-2: 49602475C/T
8-2: 49602481A/C
8-2: 49620693G/T
8-2: 49687711T/G
8-2: 49706178G/A
8-2: 49706285C/T
8-2: 49713996A/G
8-2: 49744954C/T
8-2: 49755424G/A
8-2: 49785503T/C
8-2: 49799978C/A
8-2: 49800576C/.
8-2: 49800607C/.
8-2: 49800609A/.
8-2: 49800618C/T
8-2: 49800633C/.
8-2: 49800639T/A
8-2: 49800648A/.
8-2: 49800666A/.
8-2: 49800693C/.
8-2: 49800700A/.
8-2: 49800701T/C
8-2: 49800708C/.
8-2: 49800731C/T
8-2: 49800732T/C
8-2: 49800783C/.
8-2: 49807558C/.
8-2: 49807578A/C
8-2: 49807579G/C
8-2: 49807615C/T
8-2: 49807618G/A
8-2: 49807653A/G
8-2: 49807698T/C
8-2: 49807751T/G
8-2: 49807774A/C
8-2: 49808248G/A
8-2: 49808272C/A
8-2: 49808293A/T
8-2: 49828431T/C
8-2: 49875530A/T
8-2: 49971402G/C
8-2: 50014970C/T
8-2: 50072092G/T
8-2: 50072216A/G
8-2: 50097723A/G
8-2: 50127386G/C
8-2: 50132766A/G
8-2: 50149722A/C
8-2: 50160569T/A
8-2: 50168403G/A
8-2: 50186369A/G
8-2: 50246893T/C
8-2: 50262379G/C
8-2: 50283255C/A
8-2: 50344960A/G
8-2: 50440187C/T
8-2: 50507385T/A
8-2: 50539386C/T
8-2: 50564832A/C
8-2: 50613184A/T
8-2: 50660116A/G
8-2: 50660221A/G
8-2: 50679935A/G
8-2: 50686985A/G
8-2: 50689213C/T
8-2: 50692401G/A
8-2: 50729984C/A
8-2: 50734765A/G
8-2: 50820377A/T
8-2: 50834838A/G
8-2: 50839042G/T
8-2: 50872395T/A
8-2: 50880121A/G
8-2: 50881212C/G
8-2: 50891082G/A
8-2: 50892551C/A
8-2: 50892680G/A
8-2: 50892827G/A
8-2: 50893267G/A
8-2: 50893419C/T
8-2: 50897107C/T
8-2: 50897208A/G
8-2: 50897312C/T
8-2: 50897871G/A
8-2: 50897883T/G
8-2: 50955100T/C
8-2: 50965538G/A
8-2: 50992567A/G
8-2: 51002875G/T
8-2: 51030015A/G
8-2: 51030075A/G
8-2: 51030786C/T
8-2: 51031194C/G
8-2: 51035928G/C
8-2: 51036084A/G
8-2: 51037296C/T
8-2: 51038190G/T
8-2: 51038386A/G
8-2: 51038575G/A
8-2: 51038620A/C
8-2: 51038698T/C
8-2: 51040965A/G
8-2: 51074304C/T
8-2: 51114085C/T
8-2: 51114193C/T
8-2: 51144711A/G
8-2: 51170766A/G
8-2: 51200174A/T
8-2: 51250698A/T
8-2: 51264468G/A
8-2: 51264489G/T
8-2: 51264504G/A
8-2: 51264549G/T
8-2: 51264691G/A
8-2: 51268688G/C
8-2: 51281044A/T
8-2: 51313629A/T
8-2: 51356470C/G
8-2: 51356637C/A
8-2: 51356734A/G
8-2: 51361543A/T
8-2: 51366863G/T
8-2: 51412655A/C
8-2: 51438009G/A
8-2: 51497538G/A
8-2: 51519850C/T
8-2: 51519996T/A
8-2: 51546916A/G
8-2: 51577017A/C
8-2: 51606243G/C
8-2: 51622011C/A
8-2: 51634251T/C
8-2: 51664417C/T
8-2: 51710352A/G
8-2: 51754771G/A
8-2: 51765555G/T
8-2: 51847198A/T
8-2: 51858212T/C
8-2: 51885501C/A
8-2: 51899396T/C
8-2: 51921348C/T
8-2: 51973612G/C
8-2: 51984465C/T
8-2: 51995810A/T
8-2: 52005185A/G
8-2: 52063186A/G
8-2: 52069725C/T
8-2: 52081141T/C
8-2: 52127116A/T
8-2: 52168872G/A
8-2: 52171048T/A
8-2: 52187934C/T
8-2: 52205701T/G
8-2: 52206001T/A
8-2: 52220482G/C
8-2: 52239676A/G
8-2: 52250481A/T
8-2: 52259630G/T
8-2: 52260618C/T
8-2: 52318649A/C
8-2: 52368925C/T
8-2: 52394970T/C
8-2: 52442352A/T
8-2: 52450551G/T
8-2: 52471785A/G
8-2: 52491748T/G
8-2: 52525542G/A
8-2: 52578527T/A
8-2: 52603298G/A
8-2: 52604827T/A
8-2: 52612199C/A
8-2: 52613825C/T
8-2: 52636541T/G
8-2: 52644865A/G
8-2: 52653434G/T
8-2: 52656482A/T
8-2: 52682890T/C
8-2: 52697476T/A
8-2: 52713609T/C
8-2: 52734024G/A
8-2: 52751402T/A
8-2: 52781726G/A
8-2: 52795336T/C
8-2: 52833445T/C
8-2: 52848624C/T
8-2: 52886502C/T
8-2: 52920369T/A
8-2: 52921604A/G
8-2: 52921793G/A
8-2: 52947001T/A
8-2: 53065389G/A
8-2: 53065547A/C
8-2: 53081042G/T
8-2: 53096260A/G
8-2: 53109237T/A
8-2: 53135377G/A
8-2: 53172935G/A
8-2: 53215288G/A
8-2: 53227091A/C
8-2: 53229045A/T
8-2: 53234461T/G
8-2: 53257898C/T
8-2: 53296508C/T
8-2: 53318924C/A
8-2: 53359405T/G
8-2: 53364300T/A
8-2: 53409304A/T
8-2: 53443406T/C
8-2: 53476420C/A
8-2: 53512435A/G
8-2: 53567875A/C
8-2: 53612253G/A
8-2: 53649513A/T
8-2: 53694306A/T
8-2: 53710690T/A
8-2: 53718760C/G
8-2: 53722954C/T
8-2: 53746161A/C
8-2: 53746331A/T
8-2: 53746361A/T
8-2: 53747038G/C
8-2: 53791904T/C
8-2: 53820429A/G
8-2: 53858644G/A
8-2: 53859405G/A
8-2: 53914760G/A
8-2: 53969312A/C
8-2: 54035895A/T
8-2: 54061379A/C
8-2: 54082499T/A
8-2: 54196784T/C
8-2: 54202360G/C
8-2: 54219314T/A
8-2: 54230763T/C
8-2: 54259014G/A
8-2: 54289538T/C
8-2: 54308345C/A
8-2: 54308519C/T
8-2: 54310038C/T
8-2: 54310372C/T
8-2: 54331645G/T
8-2: 54334401T/C
8-2: 54380510G/A
8-2: 54395387T/C
8-2: 54416330A/G
8-2: 54426799A/T
8-2: 54460760G/A
8-2: 54498800C/T
8-2: 54537426G/A
8-2: 54621794T/A
8-2: 54656568A/C
8-2: 54703966A/T
8-2: 54730430A/G
8-2: 54774935T/C
8-2: 54809082A/T
8-2: 54894160C/T
8-2: 54981008C/T
8-2: 55026849T/C
8-2: 55090973T/G
8-2: 55165861C/A
8-2: 55249857A/T
8-2: 55359749A/G
8-2: 55423256A/G
8-2: 55469807C/T
8-2: 55580269T/G
8-2: 55616461T/C
8-2: 55652070T/A
8-2: 55686816T/G
8-2: 55688414A/G
8-2: 55723516T/G
8-2: 55786201A/G
8-2: 55800022G/C
8-2: 55866934G/A
8-2: 55879560T/C
8-2: 55880062A/G
8-2: 55910306T/C
8-2: 55928742G/C
8-2: 55965006G/C
8-2: 56017597T/C
8-2: 56018797C/T
8-2: 56076922A/G
8-2: 56151640A/T
8-2: 56169427T/C
8-2: 56199631C/T
8-2: 56234122C/G
8-2: 56256291T/C
8-2: 56315673G/A
8-2: 56332256G/A
8-2: 56421760C/T
8-2: 56483206T/G
8-2: 56498384C/T
8-2: 56570560T/C
8-2: 56646051C/T
8-2: 56758405G/A
8-2: 56773962C/T
8-2: 56774502C/T
8-2: 56815145C/G
8-2: 56950195G/A
8-2: 57095334A/T
8-2: 57133056C/T
8-2: 57162664G/A
8-2: 57189859C/G
8-2: 57248879G/A
8-2: 57301200T/C
8-2: 57376246G/A
8-2: 57466691T/A
8-2: 57532132C/A
8-2: 57569358A/C
8-2: 57605467G/A
8-2: 57638306A/G
8-2: 57681722A/G
8-2: 57729255T/C
8-2: 57757012A/G
8-2: 57860347T/G
8-2: 57967378G/T
8-2: 57969637G/A
8-2: 58099417C/T
8-2: 58106550A/T
8-2: 58109935T/G
8-2: 58149063C/T
8-2: 58180124T/G
8-2: 58181040T/G
8-2: 58181094G/T
8-2: 58181255C/G
8-2: 58183082C/A
8-2: 58190523C/T
8-2: 58207812T/A
8-2: 58218435C/G
8-2: 58219213C/T
8-2: 58221129A/G
8-2: 58222399A/T
8-2: 58224201C/T
8-2: 58224350G/A
8-2: 58225358G/C
8-2: 58281505A/T
8-2: 58292207A/G
8-2: 58329353A/G
8-2: 58341802C/G
8-2: 58425984C/T
8-2: 58463728T/C
8-2: 58475262T/C
8-2: 58613788T/C
8-2: 58683643A/T
8-2: 58721586G/C
8-2: 58744811T/C
8-2: 58827307C/T
8-2: 58890830T/A
8-2: 58942738A/G
8-2: 59045704A/C
8-2: 59209652G/A
8-2: 59319304A/G
8-2: 59359997C/T
8-2: 59385321G/A
8-2: 59385389G/A
8-2: 59535678G/T
8-2: 59575576C/T
8-2: 59696032G/A
8-2: 59713675T/C
8-2: 59903202A/G
8-2: 60101696T/A
8-2: 60184579C/A
8-2: 60261910C/G
8-2: 60530143G/A
8-2: 60594583T/G
8-2: 60663802T/A
8-2: 60763389A/G
8-2: 60775219T/C
8-2: 60869964G/A
8-2: 60872170C/T
8-2: 60949448G/T
8-2: 60976630A/T
8-2: 61102704A/C
8-2: 61106312C/A
8-2: 61116568T/C
8-2: 61162898A/G
8-2: 61185350C/T
8-2: 61264855G/A
8-2: 61276199C/G
8-2: 61497140T/C
8-2: 61761956C/T
8-2: 61765193C/G
8-2: 61795234C/A
8-2: 61847068A/G
8-2: 61959428G/A
8-2: 62014268T/C
8-2: 62177111T/G
8-2: 62259901T/C
8-2: 62319361A/G
8-2: 62364678G/A
8-2: 62523886C/T
8-2: 62626247G/C
8-2: 62674430G/C
8-2: 62674434G/A
8-2: 62681467A/G
8-2: 62681505A/T
8-2: 62681580C/T
8-2: 62681683C/A
8-2: 62691096T/C
8-2: 62706715A/G
8-2: 62717778C/T
8-2: 62734034G/A
8-2: 62787944T/C
8-2: 62808415A/G
8-2: 62922933C/T
8-2: 62925495C/T
8-2: 62937721A/G
8-2: 62950785C/T
8-2: 62952315C/A
8-2: 62969813G/T
8-2: 63033040A/C
8-2: 63073330C/T
8-2: 63148994G/C
8-2: 63245496T/A
8-2: 63380223T/C
8-2: 63403683C/A
8-2: 63516736A/T
8-2: 63526848C/T
8-2: 63539356G/A
8-2: 63578717A/G
8-2: 63616285G/T
8-2: 63797209G/T
8-2: 64182430G/A
8-2: 64529259G/A
8-2: 64543335C/T
8-2: 64553948A/G
8-2: 64566278C/T
8-2: 64585346G/C
8-2: 64646709T/C
8-2: 64710632C/T
8-2: 64738236A/G
8-2: 64771093G/A
8-2: 64788904C/T
8-2: 64799375T/C
8-2: 64809077C/T
8-2: 64809384A/G
8-2: 64809526T/C
8-2: 64839145T/C
8-2: 64921261T/C
8-2: 65053021C/T
8-2: 65150173T/A
8-2: 65175246T/G
8-2: 65187947C/T
8-2: 65201509A/C
8-2: 65212110C/T
8-2: 65224901T/A
8-2: 65242262C/T
8-2: 65271578T/G
8-2: 65336122G/A
8-2: 65350786C/A
8-2: 65351408A/G
8-2: 65384154A/G
8-2: 65435070C/G
8-2: 65437322T/G
8-2: 65451677G/A
8-2: 65457824G/T
8-2: 65509027G/A
8-2: 65528637T/A
8-2: 65529311G/C
8-2: 65531889A/G
8-2: 65532216G/T
8-2: 65533304G/A
8-2: 65534575T/A
8-2: 65538904C/T
8-2: 65553100A/G
8-2: 65575881C/T
8-2: 65593769A/G
8-2: 65603841T/G
8-2: 65604329G/A
8-2: 65604344G/A
8-2: 65604477A/T
8-2: 65604794C/T
8-2: 65604849T/C
8-2: 65607650T/C
8-2: 65607718T/C
8-2: 65607772C/A
8-2: 65610245C/A
8-2: 65616980A/T
8-2: 65617027A/G
8-2: 65617242A/G
8-2: 65617775T/C
8-2: 65627181G/T
8-2: 65632883C/T
8-2: 65635783A/T
8-2: 65709185G/A
8-2: 65734800G/A
8-2: 65782130T/G
8-2: 65847250A/G
8-2: 65852765A/G
8-2: 65879824G/A
8-2: 65889831C/A
8-2: 65912396A/C
8-2: 65986974G/T
8-2: 66027198T/C
8-2: 66122106T/G
8-2: 66276617C/T
8-2: 66312429T/A
8-2: 66393796C/T
8-2: 66558887G/C
8-2: 66595663T/C
8-2: 66603191C/T
8-2: 66773060C/T
8-2: 66842806T/C
8-2: 66881760C/T
8-2: 66896253G/A
8-2: 66946048T/C
8-2: 66998129A/G
8-2: 67016062T/C
8-2: 67113470G/C
8-2: 67367637G/A
8-2: 67457611A/T
8-2: 67531334A/G
8-2: 67577371C/T
8-2: 67602195C/T
8-2: 67620024C/T
8-2: 67652376T/A
8-2: 67682088A/G
8-2: 67707654A/G
8-2: 67773325C/T
8-2: 67773332A/G
8-2: 67930278T/C
8-2: 67972106G/T
8-2: 68050585C/T
8-2: 68065154T/C
8-2: 68125095C/T
8-2: 68232686T/C
8-2: 68259735C/T
8-2: 68386105C/T
8-2: 68414362T/A
8-2: 68427304C/T
8-2: 68486686T/C
8-2: 68515870G/A
8-2: 68691577T/A
8-2: 68769023C/T
8-2: 68769034A/G
8-2: 68769049G/A
8-2: 68769135T/A
8-2: 68924615G/T
8-2: 68982593A/G
8-2: 69167158C/T
8-2: 69323967A/G
8-2: 69481394G/A
8-2: 69564714T/C
8-2: 69622095C/T
8-2: 69804491C/T
8-2: 69872370G/A
8-2: 69931598C/A
8-2: 69946405A/G
8-2: 69988082C/T
8-2: 70021946A/T
8-2: 70079943G/A
8-2: 70201028G/A
8-2: 70212564C/T
8-2: 70291617A/G
8-2: 70339925T/C
8-2: 70340395A/G
8-2: 70341453G/A
8-2: 70341534C/T
8-2: 70380124T/C
8-2: 70532738G/A
8-2: 70543385A/G
8-2: 70544015C/T
8-2: 70555460C/T
8-2: 70569748A/G
8-2: 70585704A/G
8-2: 70598604A/T
8-2: 70703844T/C
8-2: 70737915T/A
8-2: 71003772C/A
8-2: 71320028T/A
8-2: 71507410C/T
8-2: 71594017A/G
8-2: 71631953A/T
8-2: 71718281T/C
8-2: 71735588G/A
8-2: 71869378A/G
8-2: 71989332C/T
8-2: 71989349G/A
8-2: 72041287T/G
8-2: 72357278C/G
8-2: 72477166T/C
8-2: 72527720G/A
8-2: 72574367G/A
8-2: 72620171C/T
8-2: 72693134C/T
8-2: 72875316C/T
8-2: 73216780T/C
8-2: 73251853A/T
8-2: 73301726G/T
8-3: 10373C/A
8-3: 10437A/T
8-3: 79290T/C
8-3: 129625A/G
8-3: 285748T/C
8-3: 405891T/C
8-3: 408110C/T
8-3: 446292A/C
8-3: 701063A/G
8-3: 730097C/A
8-3: 951750A/C
8-3: 1016070C/G
8-3: 1048500G/T
8-3: 1099061T/G
8-3: 1148136A/C
8-3: 1296743A/G
8-3: 1307473A/T
8-3: 1440683G/A
8-3: 1475706G/C
8-3: 1481459A/G
8-3: 1689004A/T
8-3: 1877273C/T
8-3: 1902168A/T
8-3: 1935864C/A
8-3: 1939510C/T
8-3: 1939818G/A
8-3: 1939919C/T
8-3: 1940086C/T
8-3: 1940112G/A
8-3: 1951710C/A
8-3: 1962524G/A
8-3: 1983303C/T
8-3: 1996336T/C
8-3: 2001551G/C
8-3: 2001614A/T
8-3: 2007665C/T
8-3: 2022052T/C
8-3: 2028518G/C
8-3: 2029515T/C
8-3: 2037431T/C
8-3: 2053759G/T
8-3: 2064364G/A
8-3: 2068652A/T
8-3: 2079834T/C
8-3: 2079982A/G
8-3: 2080144T/A
8-3: 2094318T/C
8-3: 2104973T/G
8-3: 2109422A/G
8-3: 2109691T/C
8-3: 2139135C/A
8-3: 2178371T/C
8-3: 2233690T/G
8-3: 2299033G/A
8-3: 2404679A/C
8-3: 2404714T/C
8-3: 2404717C/T
8-3: 2404790G/A
8-3: 2404811G/A
8-3: 2404823G/T
8-3: 2404864C/A
8-3: 2404927C/T
8-3: 2404958C/T
8-3: 2444035A/G
8-3: 2467162T/A
8-3: 2467273C/A
8-3: 2467339G/A
8-3: 2467346G/C
8-3: 2674751C/G
8-3: 2718463T/C
8-3: 2771352A/G
8-3: 2916648A/G
8-3: 3109630T/C
8-3: 3166428C/T
8-3: 3275808A/T
8-3: 3334945G/A
8-3: 3411785G/T
8-3: 3515825C/A
8-3: 3581082C/A
8-3: 3719894T/A
8-3: 3816295G/A
8-3: 4005537C/T
8-3: 4152136T/C
8-3: 4173005T/G
8-3: 4340995A/T
8-3: 4355872G/C
8-3: 4381112A/G
8-3: 4414405T/A
8-3: 4475009T/C
8-3: 4515173C/T
8-3: 4516649C/A
8-3: 4518367C/T
8-3: 4567193C/T
8-3: 4591639G/T
8-3: 4617407T/C
8-3: 4686331A/T
8-3: 4753785G/A
8-3: 4771065G/C
8-3: 4778916C/A
8-3: 4779010A/T
8-3: 4779188A/G
8-3: 4779546C/G
8-3: 4779664G/A
8-3: 4797261T/C
8-3: 4880356A/G
8-3: 4931564A/G
8-3: 5111736T/C
8-3: 5166007C/T
8-3: 5316901C/G
8-3: 5409215G/A
8-3: 5481804T/C
8-3: 5497632T/A
8-3: 5654522A/T
8-3: 5654530T/C
8-3: 5706301C/T
8-3: 5793540T/A
8-3: 5799774T/C
8-3: 5825535G/A
8-3: 5840019A/G
8-3: 5952790T/C
8-3: 5997896A/G
8-3: 6065366A/C
8-3: 6138054G/A
8-3: 6183214G/T
8-3: 6198531C/G
8-3: 6239602T/C
8-3: 6327686C/G
8-3: 6394582T/C
8-3: 6406192C/T
8-3: 6410234A/G
8-3: 6410698G/A
8-3: 6410702T/A
8-3: 6411006C/A
8-3: 6411074T/G
8-3: 6411204C/A
8-3: 6431518C/T
8-3: 6452706A/T
8-3: 6536877A/G
8-3: 6554723T/C
8-3: 6576280T/G
8-3: 6588273T/A
8-3: 6683805C/T
8-3: 6715371C/A
8-3: 6777324T/C
8-3: 6777435G/A
8-3: 6803937G/A
8-3: 6845401T/G
8-3: 6874592A/G
8-3: 6893595G/A
8-3: 6901356A/T
8-3: 6941843T/A
8-3: 6959711T/C
8-3: 6988951C/G
8-3: 7036803T/C
8-3: 7047340C/A
8-3: 7063678G/C
8-3: 7129118C/A
8-3: 7155312T/C
8-3: 7159837T/C
8-3: 7162725A/T
8-3: 7162856T/C
8-3: 7170736A/G
8-3: 7198910T/C
8-3: 7491000C/T
8-3: 7565385T/G
8-3: 7689889A/C
8-3: 7711820C/A
8-3: 7791898T/G
8-3: 7982080T/A
8-3: 8077058T/C
8-3: 8088037T/C
8-3: 8108986G/A
8-3: 8162635T/C
8-3: 8347086A/T
8-3: 8493624C/A
8-3: 8511582G/A
8-3: 8521735T/A
8-3: 8521954T/G
8-3: 8522595A/T
8-3: 8523938A/G
8-3: 8523981C/G
8-3: 8524074G/T
8-3: 8525938A/C
8-3: 8528553G/T
8-3: 8532709A/C
8-3: 8548035C/A
8-3: 8582069A/T
8-3: 8621269T/G
8-3: 8646350G/A
8-3: 8646468A/G
8-3: 8647086C/T
8-3: 8647253C/T
8-3: 8647391A/T
8-3: 8651695A/C
8-3: 8692489T/C
8-3: 8734924G/A
8-3: 8755931C/G
8-3: 8877896G/A
8-3: 8914657G/A
8-3: 8936522G/T
8-3: 8980878T/A
8-3: 9043249T/A
8-3: 9071222T/C
8-3: 9122550C/T
8-3: 9133030A/C
8-3: 9184792T/C
8-3: 9364010T/C
8-3: 9364110T/C
8-3: 9426468C/T
8-3: 9464636G/A
8-3: 9497352A/G
8-3: 9553930A/T
8-3: 9624314T/C
8-3: 9638888A/G
8-3: 9718460T/C
8-3: 9719562C/T
8-3: 9747885C/A
8-3: 9781939A/G
8-3: 9782929T/C
8-3: 9784152G/A
8-3: 9784161A/G
8-3: 9784342T/A
8-3: 9785299T/C
8-3: 9785373C/G
8-3: 9785401G/T
8-3: 9785558T/G
8-3: 9799230T/A
8-3: 9801119A/C
8-3: 9801227A/G
8-3: 9801283C/T
8-3: 9801319G/T
8-3: 9801343T/A
8-3: 9801472G/C
8-3: 9803307C/T
8-3: 9803411A/T
8-3: 9803618T/C
8-3: 9803651C/T
8-3: 9803722G/A
8-3: 9803923G/A
8-3: 9804012G/C
8-3: 9804590A/T
8-3: 9804774C/T
8-3: 9804913G/A
8-3: 9805026A/G
8-3: 9805965A/T
8-3: 9806651A/G
8-3: 9807448A/T
8-3: 9808051T/C
8-3: 9808296G/A
8-3: 9813897G/T
8-3: 9822066G/A
8-3: 9822251T/A
8-3: 9836520C/T
8-3: 9847801G/T
8-3: 9847842T/C
8-3: 9848224G/A
8-3: 9848333T/A
8-3: 9848343A/G
8-3: 9848743G/A
8-3: 9849137T/G
8-3: 9849364A/C
8-3: 9849407A/T
8-3: 9849519A/G
8-3: 9849888A/G
8-3: 9849929G/A
8-3: 9850397C/T
8-3: 9850458C/T
8-3: 9850759T/A
8-3: 9851192G/A
8-3: 9851432C/T
8-3: 9851466G/A
8-3: 9851593A/C
8-3: 9852370G/C
8-3: 9852961A/T
8-3: 9852982A/T
8-3: 9853219A/G
8-3: 9853459A/G
8-3: 9853512A/G
8-3: 9854102G/C
8-3: 9937815C/T
8-3: 9954158C/T
8-3: 9954159T/G
8-3: 9973266T/A
8-3: 9996193A/G
8-3: 10065471T/C
8-3: 10096828G/C
8-3: 10149968T/C
8-3: 10249237A/G
8-3: 10383614G/T
8-3: 10404642G/A
8-3: 10477292A/T
8-3: 10514477T/A
8-3: 10588878T/G
8-3: 10624545C/T
8-3: 10683154C/T
8-3: 10759260G/T
8-3: 10802872C/A
8-3: 10817501T/A
8-3: 10818493A/G
8-3: 10819848A/C
8-3: 10819966C/A
8-3: 10820068C/T
8-3: 10820808C/A
8-3: 10827146T/C
8-3: 10827259T/C
8-3: 10827356A/T
8-3: 10827473C/G
8-3: 10827934G/T
8-3: 10829071C/T
8-3: 10829140C/T
8-3: 10829295A/C
8-3: 10843033A/T
8-3: 10854419G/T
8-3: 10855071T/C
8-3: 10895590C/T
8-3: 10920281C/T
8-3: 10926977G/A
8-3: 11005322T/C
8-3: 11030986C/A
8-3: 11075866C/T
8-3: 11138068C/T
8-3: 11209363A/G
8-3: 11221709A/G
8-3: 11248197T/C
8-3: 11305829C/T
8-3: 11379672T/C
8-3: 11393316T/C
8-3: 11412977C/T
8-3: 11491049G/A
8-3: 11525335A/C
8-3: 11551632T/C
8-3: 11626523G/T
8-3: 11681352G/C
8-3: 11725791T/A
8-3: 11781161A/G
8-3: 11838854T/C
8-3: 11944607G/A
8-3: 11946742C/A
8-3: 11979486G/A
8-3: 11990708A/G
8-3: 12060235A/G
8-3: 12149486G/A
8-3: 12162553T/C
8-3: 12285500C/A
8-3: 12374880A/C
8-3: 12414280T/A
8-3: 12476479T/C
8-3: 12523832A/G
8-3: 12554627T/C
8-3: 12560805A/T
8-3: 12581211T/A
8-3: 12614374G/T
8-3: 12851652A/T
8-3: 12960031C/T
8-3: 12987054G/A
8-3: 13036947G/T
8-3: 13087242C/T
8-3: 13099714A/G
8-3: 13109852T/A
8-3: 13158803C/T
8-3: 13169101A/G
8-3: 13181300C/T
8-3: 13259862C/A
8-3: 13302825G/C
8-3: 13360585A/T
8-3: 13398708G/C
8-3: 13445047G/A
8-3: 13481311G/A
8-3: 13555508A/T
8-3: 13567863A/G
8-3: 13593616T/C
8-3: 13614990G/A
8-3: 13751283T/G
8-3: 13779406T/A
8-3: 13900351A/G
8-3: 13947641C/T
8-3: 14010767T/C
8-3: 14047545A/G
8-3: 14127150G/A
8-3: 14180205T/G
8-3: 14222703T/C
8-3: 14326555T/C
8-3: 14394514C/A
8-3: 14423466C/T
8-3: 14480670A/C
8-3: 14512620G/A
8-3: 14641324T/C
8-3: 14656116T/C
8-3: 14690995C/T
8-3: 14709828A/G
8-3: 14713414A/G
8-3: 14787154T/C
8-3: 14888897C/T
8-3: 14963806T/A
8-3: 15004103A/T
8-3: 15009600A/G
8-3: 15016200G/A
8-3: 15021629T/C
8-3: 15035924G/T
8-3: 15074355C/A
8-3: 15137273T/C
8-3: 15139481G/A
8-3: 15152707G/T
8-3: 15201799C/T
8-3: 15235984T/A
8-3: 15268824C/T
8-3: 15353563C/T
8-3: 15355555T/C
8-3: 15418583A/T
8-3: 15440771C/T
8-3: 15460923C/T
8-3: 15536785G/A
8-3: 15547079C/T
8-3: 15580869A/T
8-3: 15606062A/T
8-3: 15639723G/T
8-3: 15664073A/G
8-3: 15686484C/T
8-3: 15744652G/A
8-3: 15794398G/A
8-3: 15804555C/A
8-3: 15963839C/T
8-3: 15978274C/T
8-3: 16002346C/T
8-3: 16027067A/G
8-3: 16042956T/A
8-3: 16056835A/G
8-3: 16100070A/C
8-3: 16119288T/G
8-3: 16120190T/A
8-3: 16131839A/G
8-3: 16133747T/C
8-3: 16141310A/T
8-3: 16141313C/T
8-3: 16141725C/G
8-3: 16141884A/T
8-3: 16143524C/T
8-3: 16144267A/C
8-3: 16144380G/C
8-3: 16150883T/G
8-3: 16152602T/C
8-3: 16167907T/C
8-3: 16182805T/A
8-3: 16200877A/T
8-3: 16215166G/A
8-3: 16216365A/G
8-3: 16221549G/A
8-3: 16223927G/T
8-3: 16224128C/G
8-3: 16224881T/C
8-3: 16236027A/C
8-3: 16337989T/C
8-3: 16366953A/G
8-3: 16406680G/A
8-3: 16430611G/T
8-3: 16460988T/C
8-3: 16538041G/T
8-3: 16586852C/T
8-3: 16887714G/T
8-3: 16945527A/C
8-3: 17002169A/G
8-3: 17100665G/C
8-3: 17293111C/T
8-3: 17310884C/T
8-3: 17310996G/T
8-3: 17315880T/A
8-3: 17403282T/A
8-3: 17494131C/T
8-3: 17546066A/T
8-3: 17667455T/A
8-3: 17674086C/T
8-3: 17680828C/T
8-3: 17683014G/A
8-3: 17692508T/A
8-3: 17705682C/A
8-3: 17771724T/C
8-3: 17775451A/T
8-3: 17791450G/A
8-3: 17956894G/A
8-3: 17974247T/C
8-3: 17990434T/A
8-3: 18013700C/T
8-3: 18029205C/T
8-3: 18073860T/C
8-3: 18105600T/A
8-3: 18183012C/T
8-3: 18210711G/A
8-3: 18232833G/A
8-3: 18240532G/A
8-3: 18243868C/T
8-3: 18254063A/G
8-3: 18301095T/C
8-3: 18343843A/T
8-3: 18435426A/G
8-3: 18518284C/A
8-3: 18553472G/A
8-3: 18632009T/C
8-3: 18646616G/T
8-3: 18668319C/A
8-3: 18776633C/T
8-3: 18804910C/T
8-3: 18909261G/A
8-3: 18913402G/T
8-3: 18945415C/T
8-3: 18997893T/A
8-3: 19023140C/G
8-3: 19030409T/C
8-3: 19039253G/A
8-3: 19045410T/A
8-3: 19062459A/C
8-3: 19063753T/G
8-3: 19065963G/A
8-3: 19068232A/G
8-3: 19075485G/A
8-3: 19089085G/T
8-3: 19107379G/A
8-3: 19107396A/G
8-3: 19108492G/T
8-3: 19120082A/G
8-3: 19152910T/C
8-3: 19189747A/C
8-3: 19267500T/A
8-3: 19299676T/C
8-3: 19355572T/A
8-3: 19382616A/G
8-3: 19409541C/T
8-3: 19427133T/A
8-3: 19430783C/T
8-3: 19460368A/G
8-3: 19508892G/A
8-3: 19594753C/A
8-3: 19675486G/A
8-3: 19722054C/T
8-3: 19792493C/T
8-3: 19823619T/C
8-3: 19862287C/T
8-3: 19963231C/T
8-3: 20028870A/G
8-3: 20065985C/A
8-3: 20122507A/T
8-3: 20207170C/T
8-3: 20221239G/A
8-3: 20232722A/G
8-3: 20357900A/C
8-3: 20378958G/A
8-3: 20397703G/T
8-3: 20449911C/T
8-3: 20537580C/T
8-3: 20561902A/C
8-3: 20596489T/A
8-3: 20616196T/C
8-3: 20625201T/A
8-3: 20683131T/C
8-3: 20717335T/A
8-3: 20800798C/T
8-3: 20830668T/C
8-3: 20873221G/A
8-3: 20874623T/C
8-3: 20874750G/A
8-3: 20905651A/G
8-3: 20920082C/A
8-3: 20949183G/A
8-3: 21001671G/A
8-3: 21027345G/A
8-3: 21073400A/T
8-3: 21073458G/C
8-3: 21073509G/A
8-3: 21075041A/C
8-3: 21075199A/G
8-3: 21075226T/C
8-3: 21075429G/A
8-3: 21075614G/A
8-3: 21075680A/T
8-3: 21075899C/T
8-3: 21075901G/A
8-3: 21077960G/A
8-3: 21078110T/C
8-3: 21079103C/A
8-3: 21079498G/A
8-3: 21081249T/C
8-3: 21081383G/T
8-3: 21081526C/T
8-3: 21081635G/T
8-3: 21081642T/A
8-3: 21081819G/A
8-3: 21081989G/A
8-3: 21082114T/A
8-3: 21095305A/G
8-3: 21103330A/C
8-3: 21103420T/C
8-3: 21103588C/T
8-3: 21103975A/T
8-3: 21104043T/C
8-3: 21104311T/A
8-3: 21104412A/T
8-3: 21104788G/T
8-3: 21104829T/C
8-3: 21105365T/C
8-3: 21105457A/G
8-3: 21105521G/A
8-3: 21105625A/G
8-3: 21105742T/G
8-3: 21105966A/G
8-3: 21108295G/A
8-3: 21108371T/G
8-3: 21108481G/A
8-3: 21116635A/G
8-3: 21117997A/G
8-3: 21118114T/C
8-3: 21120147C/T
8-3: 21147784G/A
8-3: 21147822A/C
8-3: 21148796C/T
8-3: 21154632T/C
8-3: 21213116T/A
8-3: 21246586C/G
8-3: 21274080T/A
8-3: 21289898T/C
8-3: 21323073C/T
8-3: 21355130T/C
8-3: 21386443T/C
8-3: 21424344C/G
8-3: 21434692A/G
8-3: 21448167G/T
8-3: 21461122A/C
8-3: 21471710G/A
8-3: 21508706G/A
8-3: 21518861C/G
8-3: 21525799T/C
8-3: 21526490A/T
8-3: 21543968G/T
8-3: 21601769C/A
8-3: 21611945G/A
8-3: 21618867A/G
8-3: 21656727A/T
8-3: 21707495T/C
8-3: 21748571C/T
8-3: 21771602G/T
8-3: 21791524G/A
8-3: 21922571A/C
8-3: 21999696C/A
8-3: 22058729C/A
8-3: 22087863G/A
8-3: 22154894A/G
8-3: 22211852T/C
8-3: 22330361T/C
8-3: 22417562A/C
8-3: 22418496A/T
8-3: 22428825G/A
8-3: 22450530T/C
8-3: 22468718A/G
8-3: 22484543T/A
8-3: 22504701C/T
8-3: 22539370T/G
8-3: 22645912T/C
8-3: 22658726T/G
8-3: 22683056G/A
8-3: 22698807A/G
8-3: 22721880G/A
8-3: 22818406C/T
8-3: 22832680G/A
8-3: 22854751A/G
8-3: 22901995A/G
8-3: 22902405T/A
8-3: 22917703G/T
8-3: 22964507G/A
8-3: 23011464C/A
8-3: 23095671G/A
8-3: 23121943C/A
8-3: 23151658A/C
8-3: 23152623C/A
8-3: 23152789A/T
8-3: 23167295A/T
8-3: 23178352G/A
8-3: 23240664A/T
8-3: 23314113T/C
8-3: 23367277T/C
8-3: 23398887T/A
8-3: 23439824T/A
8-3: 23483052C/T
8-3: 23514424A/G
8-3: 23677748G/C
8-3: 23689051C/T
8-3: 23689993T/G
8-3: 23707066G/A
8-3: 23720032T/A
8-3: 23721582G/T
8-3: 23722867G/A
8-3: 23813707A/T
8-3: 23860213T/C
8-3: 23905447T/A
8-3: 23976746G/A
8-3: 23989960C/T
8-3: 24082406A/G
8-3: 24112644C/T
8-3: 24143442A/G
8-3: 24183887T/G
8-3: 24255753C/T
8-3: 24274287G/A
8-3: 24285285T/G
8-3: 24328982C/T
8-3: 24365482T/C
8-3: 24433349G/A
8-3: 24490752A/G
8-3: 24613500G/A
8-3: 24693323T/G
8-3: 24718778A/T
8-3: 24733086T/G
8-3: 24836533T/A
8-3: 24873369T/C
8-3: 24884469G/A
8-3: 24884955C/A
8-3: 24889587T/A
8-3: 25081028A/G
8-3: 25125447T/G
8-3: 25191141G/T
8-3: 25203325C/T
8-3: 25274743C/A
8-3: 25311121G/A
8-3: 25341632G/T
8-3: 25366811T/C
8-3: 25383984A/G
8-3: 25385039C/T
8-3: 25403292A/T
8-3: 25433209G/A
8-3: 25491358C/T
8-3: 25532240T/C
8-3: 25532376G/A
8-3: 25532856A/G
8-3: 25543870G/A
8-3: 25543881G/A
8-3: 25544024G/A
8-3: 25567158A/T
8-3: 25710359A/T
8-3: 25773282C/T
8-3: 25784874G/C
8-3: 25873581T/C
8-3: 25974301A/G
8-3: 26009374C/G
8-3: 26039121C/T
8-3: 26093679C/T
8-3: 26094356C/T
8-3: 26094381C/T
8-3: 26157357G/T
8-3: 26175036G/A
8-3: 26185951G/A
8-3: 26208018C/A
8-3: 26227675G/A
8-3: 26282934C/T
8-3: 26286828T/C
8-3: 26301937A/T
8-3: 26391986C/T
8-3: 26394446T/C
8-3: 26405225T/G
8-3: 26421981G/A
8-3: 26434586A/G
8-3: 26437618G/A
8-3: 26437696G/A
8-3: 26445445T/C
8-3: 26475925T/A
8-3: 26507055C/T
8-3: 26536326C/T
8-3: 26603032T/A
8-3: 26681041A/T
8-3: 26686289A/T
8-3: 26724231C/T
8-3: 26803744A/C
8-3: 26847173G/A
8-3: 26892470C/T
8-3: 27095406C/G
8-3: 27122340C/T
8-3: 27216366T/A
8-3: 27307466A/G
8-3: 27309225G/T
8-3: 27328321T/A
8-3: 27357442C/G
8-3: 27371969T/C
8-3: 27372670C/A
8-3: 27485443G/A
8-3: 27486881G/A
8-3: 27511218G/T
8-3: 27736231G/A
8-3: 27799083T/G
8-3: 27850477G/A
8-3: 27853218C/A
8-3: 27863041A/T
8-3: 27864703C/T
8-3: 27876570A/G
8-3: 27979471C/A
8-3: 27982985T/C
8-3: 27987896A/T
8-3: 27998157C/T
8-3: 28016211G/T
8-3: 28041659C/T
8-3: 28056424G/A
8-3: 28072359G/A
8-3: 28164948A/T
8-3: 28214854A/G
8-3: 28215362A/G
8-3: 28215365C/G
8-3: 28227289T/G
8-3: 28278076A/T
8-3: 28345149C/A
8-3: 28490555A/G
8-3: 28578314T/C
8-3: 28615325T/A
8-3: 28711936G/T
8-3: 28714929G/C
8-3: 28751929A/C
8-3: 28773004T/A
8-3: 28826421C/T
8-3: 28912043T/C
8-3: 28923678A/C
8-3: 28931209A/G
8-3: 28953605C/T
8-3: 29095379C/T
8-3: 29134200G/A
8-3: 29153035C/T
8-3: 29223914C/T
8-3: 29224419T/G
8-3: 29238061C/G
8-3: 29313566A/G
8-3: 29572732G/A
8-3: 29835207G/A
8-3: 29912586T/C
8-3: 30083541T/C
8-3: 30097552A/G
8-3: 30098583A/G
8-3: 30325599G/A
8-3: 30442632T/A
8-3: 30588365C/T
8-3: 30678035T/C
8-3: 30782031T/C
8-3: 31095199T/A
8-3: 31306222A/G
8-3: 31445538A/T
8-3: 31490013C/G
8-3: 31711366A/G
8-3: 31724322G/A
8-3: 31930489G/T
8-3: 31952663T/A
8-3: 31983950G/T
8-3: 32010845A/C
8-3: 32021648T/C
8-3: 32032902T/C
8-3: 32045420A/G
8-3: 32055684T/G
8-3: 32066218C/T
8-3: 32070376G/C
8-3: 32074222C/T
8-3: 32074937G/T
8-3: 32074941A/G
8-3: 32074981G/A
8-3: 32075013T/C
8-3: 32083787A/G
8-3: 32103642T/A
8-3: 32104739G/C
8-3: 32120417T/C
8-3: 32147894C/A
8-3: 32185523A/G
8-3: 32186644T/C
8-3: 32222426C/T
8-3: 32266553T/C
8-3: 32284188C/G
8-3: 32289270A/T
8-3: 32422150C/T
8-3: 32440805G/A
8-3: 32549814C/T
8-3: 32550602G/A
8-3: 32555981A/T
8-3: 32566526G/A
8-3: 32584015A/G
8-3: 32819480A/C
8-3: 32829859C/T
8-3: 32890952A/G
8-3: 32893820C/T
8-3: 32894399A/G
8-3: 32894656G/A
8-3: 32897143G/T
8-3: 32905314G/A
8-3: 32923363C/T
8-3: 32950127G/A
8-3: 32972051C/T
8-3: 32987163G/T
8-3: 33101078T/A
8-3: 33145567G/A
8-3: 33174432T/A
8-3: 33177269G/T
8-3: 33284321C/T
8-3: 33388712G/T
8-3: 33534520T/A
8-3: 33671504G/A
8-3: 33684477G/T
8-3: 33813532A/G
8-3: 33816020T/A
8-3: 33855176C/T
8-3: 33856837A/T
8-3: 33856939C/T
8-3: 33867343T/C
8-3: 33879666A/G
8-3: 33880042C/G
8-3: 33927778G/T
8-3: 33952889A/G
8-3: 33988483T/C
8-3: 34005635G/A
8-3: 34032732T/C
8-3: 34058245A/G
8-3: 34085315G/A
8-3: 34254499T/C
8-3: 34254504C/T
8-3: 34254549T/G
8-3: 34254553A/G
8-3: 34254558T/C
8-3: 34254585G/A
8-3: 34254589G/A
8-3: 34254618A/T
8-3: 34254625G/T
8-3: 34254627A/G
8-3: 34255562A/G
8-3: 34262552G/A
8-3: 34279932T/C
8-3: 34331706C/T
8-3: 34365299A/C
8-3: 34412336G/A
8-3: 34422930T/C
8-3: 34427529C/A
8-3: 34427906T/C
8-3: 34446422A/C
8-3: 34465940T/A
8-3: 34476799C/T
8-3: 34477062A/G
8-3: 34477247C/T
8-3: 34478011A/T
8-3: 34479283T/C
8-3: 34488098T/A
8-3: 34490995A/G
8-3: 34491013T/A
8-3: 34505039T/A
8-3: 34591601C/T
8-3: 34731663T/C
8-3: 34812699G/T
8-3: 34829162G/T
8-3: 34936845T/G
8-3: 35034545T/C
8-3: 35073595G/A
8-3: 35123736A/G
8-3: 35141416A/T
8-3: 35216052A/T
8-3: 35238906T/G
8-3: 35266755C/A
8-3: 35308225G/C
8-3: 35338139G/A
8-3: 35339132C/A
8-3: 35416534G/A
8-3: 35435229G/A
8-3: 35456166A/G
8-3: 35484805C/T
8-3: 35516189A/G
8-3: 35588142T/C
8-3: 35611212C/A
8-3: 35612679A/G
8-3: 35613035G/C
8-3: 35622048A/G
8-3: 35622308G/A
8-3: 35634165C/T
8-3: 35743616T/G
8-3: 35801805A/T
8-3: 35801811C/G
8-3: 35877792G/C
8-3: 35984760T/C
8-3: 36466211A/T
8-3: 36650778C/G
8-3: 36807183A/T
8-3: 36836900C/G
8-3: 36938685T/A
8-3: 36951155A/G
8-3: 36992584G/A
8-3: 37030513G/A
8-3: 37045338C/T
8-3: 37065274T/C
8-3: 37238100A/C
8-3: 37252880C/G
8-3: 37475386T/C
8-3: 37491348A/T
8-3: 37520959A/G
8-3: 37599764T/A
8-3: 37623950A/T
8-3: 37642442C/A
8-3: 37643058G/A
8-3: 37643143G/A
8-3: 37679548G/C
8-3: 37707120A/G
8-3: 37707758A/T
8-3: 37730153G/C
8-3: 37748884T/A
8-3: 37768531C/T
8-3: 37780140T/C
8-3: 37799255T/A
8-3: 37846128C/T
8-3: 37966605A/G
8-3: 37976629A/T
8-3: 37999313A/C
8-3: 38169482C/T
8-3: 38248726A/T
8-3: 38343160G/A
8-3: 38385909G/A
8-3: 38401051A/G
8-3: 38420008C/T
8-3: 38443754C/A
8-3: 38463228A/C
8-3: 38545523T/C
8-3: 38577445A/G
8-3: 38625977T/A
8-3: 38748494A/G
8-3: 38760530C/T
8-3: 38760804G/C
8-3: 38760964A/C
8-3: 38763051C/T
8-3: 38763097T/C
8-3: 38763525G/A
8-3: 38764082T/G
8-3: 38764093C/A
8-3: 38771406A/G
8-3: 38816577C/T
8-3: 38829898T/C
8-3: 38866402C/A
8-3: 38908640C/T
8-3: 38908789C/G
8-3: 38908808A/G
8-3: 38908842G/A
8-3: 38908965A/C
8-3: 38908970T/C
8-3: 38908988T/G
8-3: 38909039C/T
8-3: 38909047A/G
8-3: 38909051A/T
8-3: 38966696C/A
8-3: 39114636A/T
8-3: 39120486G/A
8-3: 39209528C/T
8-3: 39222139T/G
8-3: 39262049A/C
8-3: 39308336C/G
8-3: 39318901T/C
8-3: 39389062G/T
8-3: 39413723A/G
8-3: 39414707T/C
8-3: 39425728C/A
8-3: 39426875G/T
8-3: 39427287G/A
8-3: 39427299T/A
8-3: 39427395T/C
8-3: 39464955C/T
8-3: 39483514T/C
8-3: 39498895T/A
8-3: 39656526T/C
8-3: 39660560A/C
8-3: 39670984T/C
8-3: 39710029T/C
8-3: 39711472C/T
8-3: 39711659A/G
8-3: 39724333G/A
8-3: 39735587G/C
8-3: 39767935A/G
8-3: 39771450T/C
8-3: 39787913G/T
8-3: 39791558C/T
8-3: 39816200T/G
8-3: 39826651G/A
8-3: 39949302A/G
8-3: 40181454T/G
8-3: 40285844G/A
8-3: 40377062A/C
8-3: 40378230T/A
8-3: 40394687C/T
8-3: 40430740G/T
8-3: 40464757T/A
8-3: 40502191A/T
8-3: 40512556A/G
8-3: 40595453G/T
8-3: 40609398A/G
8-3: 40615295C/T
8-3: 40615551T/A
8-3: 40629878G/A
8-3: 40635712A/T
8-3: 40743615G/A
8-3: 40744728C/T
8-3: 40748105A/G
8-3: 40764536C/T
8-3: 40770679A/C
8-3: 40770730T/A
8-3: 40771028C/A
8-3: 40772199T/A
8-3: 40773688T/C
8-3: 40774665A/G
8-3: 40774791T/C
8-3: 40778369C/G
8-3: 40843919C/T
8-3: 40881710C/T
8-3: 40901172A/T
8-3: 40918633T/C
8-3: 40935755G/T
8-3: 40961161C/T
8-3: 40996005T/C
8-3: 41042016A/T
8-3: 41055621C/A
8-3: 41087388C/T
8-3: 41132895C/A
8-3: 41238145A/C
8-3: 41253482C/T
8-3: 41265313G/T
8-3: 41267757A/T
8-3: 41270721C/T
8-3: 41315019A/G
8-3: 41316735C/T
8-3: 41346133T/C
8-3: 41346510T/G
8-3: 41362545C/T
8-3: 41375962A/C
8-3: 41375974C/T
8-3: 41376339C/G
8-3: 41376376G/A
8-3: 41376381A/C
8-3: 41376401G/A
8-3: 41483436C/G
8-3: 41555206T/G
8-3: 41605265A/G
8-3: 41634035C/T
8-3: 41666680A/G
8-3: 41709944G/A
8-3: 41746717T/C
8-3: 41840872G/A
8-3: 41898018T/G
8-3: 41906630A/G
8-3: 41929227G/A
8-3: 41952105G/A
8-3: 41991922G/A
8-3: 42011606A/G
8-3: 42121480A/G
8-3: 42143368C/G
8-3: 42156961C/T
8-3: 42178295A/T
8-3: 42202246G/A
8-3: 42220964A/T
8-3: 42262586C/T
8-3: 42324092C/T
8-3: 42410740C/T
8-3: 42450221G/A
8-3: 42465967A/T
8-3: 42487433T/C
8-3: 42504620T/G
8-3: 42505865A/T
8-3: 42527163T/C
8-3: 42527276T/G
8-3: 42613622A/T
8-3: 42665141C/G
8-3: 42675717C/T
8-3: 42805532C/T
8-3: 42845918C/T
8-3: 42913867A/G
8-3: 42954843T/C
8-3: 43006370C/T
8-3: 43089501G/A
8-3: 43143819T/C
8-3: 43230071A/C
8-3: 43257200G/A
8-3: 43283812C/T
8-3: 43372105G/A
8-3: 43478085C/T
8-3: 43601744T/A
8-3: 43625893G/A
8-3: 43664052C/T
8-3: 43674574G/A
8-3: 43714209G/A
8-3: 43744368A/G
8-3: 43744557A/T
8-3: 43744672T/G
8-3: 43760863C/T
8-3: 43893870C/T
8-3: 43943977C/T
8-3: 43986318T/C
8-3: 43998503T/C
8-3: 44016742C/T
8-3: 44030942T/C
8-3: 44045132G/A
8-3: 44118654G/T
8-3: 44203634C/A
8-3: 44205039C/G
8-3: 44223404G/A
8-3: 44235878A/T
8-3: 44257699T/A
8-3: 44282323T/C
8-3: 44282381T/C
8-3: 44304842G/C
8-3: 44312071T/C
8-3: 44343748C/A
8-3: 44369093T/C
8-3: 44388700G/A
8-3: 44419415A/G
8-3: 44419910G/A
8-3: 44420941A/T
8-3: 44422037T/C
8-3: 44422464A/T
8-3: 44422588T/C
8-3: 44431381A/G
8-3: 44434270T/G
8-3: 44442949A/G
8-3: 44587808C/T
8-3: 44762614G/A
8-3: 44851866C/T
8-3: 44901822T/C
8-3: 44912827C/T
8-3: 45040971A/C
8-3: 45043763C/T
8-3: 45051794C/G
8-3: 45052311C/A
8-3: 45052872A/G
8-3: 45053640T/G
8-3: 45053691C/T
8-3: 45054030G/A
8-3: 45054842C/G
8-3: 45055044A/T
8-3: 45055223A/G
8-3: 45055225C/T
8-3: 45055331A/G
8-3: 45055512T/C
8-3: 45058901C/T
8-3: 45064061C/T
8-3: 45115499A/T
8-3: 45163901A/G
8-3: 45228824C/T
8-3: 45229578G/A
8-3: 45306098T/G
8-3: 45441319C/A
8-3: 45481754A/G
8-3: 45617367A/T
8-3: 45677272C/T
8-3: 45747938C/T
8-3: 45838286A/G
8-3: 45909398C/G
8-3: 46077903A/T
8-3: 46099455G/A
8-3: 46137149A/T
8-3: 46198520A/T
8-3: 46229201G/A
8-3: 46256624T/C
8-3: 46303901C/T
8-3: 46333002T/A
8-3: 46341805T/A
8-3: 46370915G/A
8-3: 46398767G/C
8-3: 46448906G/A
8-3: 46496394T/C
8-3: 46588054A/G
8-3: 46646112T/C
8-3: 46665856T/C
8-3: 46687044A/G
8-3: 46697823T/G
8-3: 46754162C/A
8-3: 46776238G/C
8-3: 46786734G/A
8-3: 46803647C/T
8-3: 46859155A/C
8-3: 46880795C/T
8-3: 46914918T/C
8-3: 46979760T/C
8-3: 47073003T/C
8-3: 47115766T/C
8-3: 47120648A/T
8-3: 47120743G/C
8-3: 47120777G/A
8-3: 47121898G/A
8-3: 47127021C/T
8-3: 47127859C/G
8-3: 47128165G/A
8-3: 47148009G/A
8-3: 47157158G/C
8-3: 47162612T/A
8-3: 47162652A/G
8-3: 47164586C/T
8-3: 47165010T/A
8-3: 47165014A/T
8-3: 47165064C/A
8-3: 47166541A/G
8-3: 47227633A/T
8-3: 47264392T/A
8-3: 47340231A/G
8-3: 47351747C/A
8-3: 47412119G/A
8-3: 47413193A/T
8-3: 47417156G/A
8-3: 47418912G/A
8-3: 47427246A/G
8-3: 47444366A/C
8-3: 47468926G/A
8-3: 47528099C/T
8-3: 47576520C/T
8-3: 47587179C/T
8-3: 47597288G/A
8-3: 47634660T/G
8-3: 47673526T/C
8-3: 47738335A/T
8-3: 47750341A/T
8-3: 47751795T/C
8-3: 47766347T/C
8-3: 47784503C/A
8-3: 47839341A/T
8-3: 47866756T/G
8-3: 47886751G/A
8-3: 47905040G/T
8-3: 47907295C/T
8-3: 47926690T/A
8-3: 47938730T/C
8-3: 47956309A/G
8-3: 47979264C/T
8-3: 47982055C/A
8-3: 47982168G/A
8-3: 47982258G/A
8-3: 47982605G/A
8-3: 47992626A/T
8-3: 47998306T/G
8-3: 47998408C/A
8-3: 47998419A/C
8-3: 47998569T/C
8-3: 47998692C/A
8-3: 47998805A/G
8-3: 48010953C/T
8-3: 48021320T/C
8-3: 48036720G/A
8-3: 48056679G/T
8-3: 48070802T/A
8-3: 48082785G/A
8-3: 48100095T/C
8-3: 48115929C/T
8-3: 48160277G/T
8-3: 48174087C/T
8-3: 48200463A/C
8-3: 48207875G/C
8-3: 48248970G/A
8-3: 48250493T/C
8-3: 48250786A/T
8-3: 48310218C/T
8-3: 48326958A/G
8-3: 48334306G/A
8-3: 48334412T/C
8-3: 48340610A/C
8-3: 48340924C/T
8-3: 48341086T/C
8-3: 48341473C/T
8-3: 48341476G/A
8-3: 48347728A/G
8-3: 48352869C/G
8-3: 48362416T/C
8-3: 48365878G/T
8-3: 48373406T/G
8-3: 48387441T/C
8-3: 48401195G/C
8-3: 48413290A/C
8-3: 48413748G/A
8-3: 48423893A/T
8-3: 48428489G/A
8-3: 48428627G/A
8-3: 48428763T/C
8-3: 48444039G/T
8-3: 48465751T/C
8-3: 48477386G/A
8-3: 48500945G/A
8-3: 48511780T/G
8-3: 48516956T/G
8-3: 48517182G/A
8-3: 48522403A/C
8-3: 48547421A/T
8-3: 48604207T/C
8-3: 48608684C/T
8-3: 48608829G/A
8-3: 48627619C/T
8-3: 48656132G/A
8-3: 48661478A/G
8-3: 48696886T/A
8-3: 48723174G/A
8-3: 48723342A/C
8-3: 48723607C/T
8-3: 48723610C/A
8-3: 48723679G/A
8-3: 48728314C/T
8-3: 48730247C/T
8-3: 48730254G/A
8-3: 48730560G/A
8-3: 48730600A/G
8-3: 48794874C/A
8-3: 48833273G/A
8-3: 48864029C/T
8-3: 48890115T/G
8-3: 48921535A/C
8-3: 48925083T/G
8-3: 48925164T/G
8-3: 48925725A/T
8-3: 48925853A/G
8-3: 48926260C/A
8-3: 48932895C/T
8-3: 48935031C/T
8-3: 48959566C/A
8-3: 49010825G/T
8-3: 49022050T/G
8-3: 49044970G/A
8-3: 49058519T/C
8-3: 49117313G/T
8-3: 49133672C/A
8-3: 49174263C/T
8-3: 49184593T/G
8-3: 49188194T/C
8-3: 49212215G/T
8-3: 49219097A/T
8-3: 49219111A/G
8-3: 49223589A/G
8-3: 49240817T/C
8-3: 49271964T/C
8-3: 49273778T/G
8-3: 49318993T/G
8-3: 49332938C/T
8-3: 49390764C/T
8-3: 49427968C/T
8-3: 49434834C/G
8-3: 49434956G/C
8-3: 49456029G/A
8-3: 49478491C/G
8-3: 49519368G/A
8-3: 49534572C/T
8-3: 49561094C/T
8-3: 49593399T/C
8-3: 49606386A/G
8-3: 49714225C/T
8-3: 49748788C/T
8-3: 49768548A/G
8-3: 49768993A/C
8-3: 49784831C/T
8-3: 49784843A/G
8-3: 49788805G/A
8-3: 49794434A/G
8-3: 49808857A/G
8-3: 49826288T/G
8-3: 49855687C/G
8-3: 49885738T/A
8-3: 49897845G/A
8-3: 49947474C/T
8-3: 49975826C/T
8-3: 50013136A/G
8-3: 50063632C/T
8-3: 50096350C/A
8-3: 50147581T/C
8-3: 50186634A/T
8-3: 50224477T/G
8-3: 50276308T/C
8-3: 50315918C/A
8-3: 50358456T/G
8-3: 50358587A/C
8-3: 50364383T/A
8-3: 50365415T/C
8-3: 50368712C/T
8-3: 50372238A/G
8-3: 50387986G/A
8-3: 50421047G/A
8-3: 50442968C/T
8-3: 50444923T/C
8-3: 50445072C/A
8-3: 50445283T/G
8-3: 50453190C/T
8-3: 50480012T/C
8-3: 50494061A/G
8-3: 50546262G/T
8-3: 50549766T/A
8-3: 50597516C/A
8-3: 50601059A/G
8-3: 50602758C/T
8-3: 50603130G/T
8-3: 50603309G/A
8-3: 50616856A/G
8-3: 50635146A/C
8-3: 50659090G/T
8-3: 50692925C/T
8-3: 50725146G/A
8-3: 50760418C/T
8-3: 50774289T/A
8-3: 50789792A/G
8-3: 50933033C/A
8-3: 50963145G/A
8-3: 50977113T/C
8-3: 51019188T/G
8-3: 51052438A/T
8-3: 51097490G/C
8-3: 51121974G/A
8-3: 51134023C/T
8-3: 51180792G/T
8-3: 51211950A/C
8-3: 51216771A/T
8-3: 51219182T/G
8-3: 51240806G/A
8-3: 51240918C/G
8-3: 51295223C/T
8-3: 51342613G/A
8-3: 51381218T/A
8-3: 51411321G/A
8-3: 51413607C/T
8-3: 51445817G/A
8-3: 51468128G/A
8-3: 51512430C/T
8-3: 51514565T/C
8-3: 51514577T/G
8-3: 51514596C/T
8-3: 51514626A/G
8-3: 51514669A/T
8-3: 51514872A/T
8-3: 51530935G/C
8-3: 51562044G/A
8-3: 51573979A/T
8-3: 51604736T/A
8-3: 51605210G/A
8-3: 51605386A/C
8-3: 51616793A/G
8-3: 51642981T/G
8-3: 51660080C/A
8-3: 51683114A/G
8-3: 51732283C/T
8-3: 51736853T/G
8-3: 51738048G/A
8-3: 51738094A/G
8-3: 51748340T/C
8-3: 51770761C/G
8-3: 51779394T/C
8-3: 51793001G/A
8-3: 51811410T/A
8-3: 51831041C/T
8-3: 51831406C/G
8-3: 51831518C/T
8-3: 51831549A/C
8-3: 51835155C/G
8-3: 51835404A/T
8-3: 51835549T/C
8-3: 51835660G/T
8-3: 51835776A/G
8-3: 51845431G/A
8-3: 51854005T/C
8-3: 51868566A/G
8-3: 51893795T/G
8-3: 51969027C/A
8-3: 51971000T/C
8-3: 51972247G/T
8-3: 51993146A/G
8-3: 52018484T/C
8-3: 52066754A/G
8-3: 52077928C/T
8-3: 52129520C/T
8-3: 52196961G/A
8-3: 52249634A/G
8-3: 52277738A/T
8-3: 52279169A/T
8-3: 52303748T/A
8-3: 52315012T/A
8-3: 52327894T/A
8-3: 52339275G/C
8-3: 52357178G/A
8-3: 52391828T/G
8-3: 52434389A/G
8-3: 52528582C/T
8-3: 52620725G/T
8-3: 52620756T/G
8-3: 52621220T/C
8-3: 52622236C/A
8-3: 52626049T/A
8-3: 52627484G/A
8-3: 52632284A/G
8-3: 52632573G/C
8-3: 52645853T/A
8-3: 52719554G/C
8-3: 52731629C/A
8-3: 52750831C/G
8-3: 52766938C/T
8-3: 52783408C/T
8-3: 52797162C/T
8-3: 52829959C/G
8-3: 52879530T/C
8-3: 52896020T/A
8-3: 52938540A/G
8-3: 52971277G/A
8-3: 52998264G/A
8-3: 52998745G/C
8-3: 53017706C/A
8-3: 53054722A/T
8-3: 53056700T/A
8-3: 53125004G/A
8-3: 53154047G/A
8-3: 53220051T/C
8-3: 53265717T/C
8-3: 53308656T/C
8-3: 53356449T/A
8-3: 53376836G/A
8-3: 53390409C/A
8-3: 53416446G/A
8-3: 53446328A/C
8-3: 53463539A/T
8-3: 53469454C/T
8-3: 53473991A/G
8-3: 53479646G/A
8-3: 53490023C/T
8-3: 53509103T/C
8-3: 53522401G/C
8-3: 53535735T/C
8-3: 53569464C/T
8-3: 53594686T/C
8-3: 53629544G/A
8-3: 53650853A/G
8-3: 53684246G/T
8-3: 53721191G/A
8-3: 53760850C/T
8-3: 53776739C/T
8-3: 53819550A/G
8-3: 53861088T/G
8-3: 53874010C/A
8-3: 53920805C/A
8-3: 53933880A/G
8-3: 53935935C/G
8-3: 53938874C/T
8-3: 53946182G/A
8-3: 53951281A/G
8-3: 53952358T/C
8-3: 53952460C/T
8-3: 53952476T/A
8-3: 53964652T/C
8-3: 53971345A/C
8-3: 53993408T/C
8-3: 54032273C/G
8-3: 54044458T/C
8-3: 54048955A/G
8-3: 54051831C/A
8-3: 54153252T/A
8-3: 54167499T/C
8-3: 54181081T/C
8-3: 54192989T/C
8-3: 54193073C/A
8-3: 54193178T/C
8-3: 54193569A/C
8-3: 54215478G/A
8-3: 54219077T/C
8-3: 54261174T/C
8-3: 54300053A/C
8-3: 54325377A/C
8-3: 54357489A/T
8-3: 54388957G/T
8-3: 54416941C/T
8-3: 54462236T/C
8-3: 54462473A/G
8-3: 54489826G/A
8-3: 54533130G/A
8-3: 54535947C/T
8-3: 54536388G/A
8-3: 54565102T/C
8-3: 54605818T/C
8-3: 54651292T/A
8-3: 54730962C/G
8-3: 54742554A/G
8-3: 54768528A/G
8-3: 54768616T/C
8-3: 54793149A/G
8-3: 54826412A/C
8-3: 54870598A/T
8-3: 54901070G/C
8-3: 54924244T/C
8-3: 55033538C/T
8-3: 55072234A/T
8-3: 55129230C/G
8-3: 55218116C/A
8-3: 55290851T/C
8-3: 55315998T/A
8-3: 55370231C/T
8-3: 55371415C/T
8-3: 55393909C/G
8-3: 55443704T/C
8-3: 55449251A/T
8-3: 55471626T/C
8-3: 55491529G/A
8-3: 55534643T/A
8-3: 55593322C/T
8-3: 55626350G/C
8-3: 55662723C/A
8-3: 55707477G/C
8-3: 55775667T/A
8-3: 55814939C/T
8-3: 55815052T/C
8-3: 55818888C/G
8-3: 55827799G/T
8-3: 55829339C/A
8-3: 55829340A/T
8-3: 55829787C/T
8-3: 55830187C/T
8-3: 55830411A/G
8-3: 55922584T/A
8-3: 55968856G/A
8-3: 56005526A/G
8-3: 56086686G/A
8-3: 56123736G/T
8-3: 56137007T/C
8-3: 56149436A/G
8-3: 56204190T/G
8-3: 56345484G/A
8-3: 56415444G/C
8-3: 56425535T/A
8-3: 56427658C/A
8-3: 56427996C/T
8-3: 56428099G/A
8-3: 56428213T/C
8-3: 56438443G/A
8-3: 56440263G/A
8-3: 56454341G/A
8-3: 56487204A/T
8-3: 56526222G/A
8-3: 56552853C/T
8-3: 56623595C/A
8-3: 56629282T/C
8-3: 56629369A/G
8-3: 56630141T/G
8-3: 56665655C/T
8-3: 56731589C/T
8-3: 56737947C/T
8-3: 56745048A/T
8-3: 56752697T/C
8-3: 56755280G/T
8-3: 56755833G/A
8-3: 56755885G/T
8-3: 56755953G/A
8-3: 56780099A/T
8-3: 56795325C/T
8-3: 56901062T/C
8-3: 57016634A/T
8-3: 57062919C/T
8-3: 57105449A/T
8-3: 57157558A/G
8-3: 57235827G/A
8-3: 57492964A/G
8-3: 57531863T/C
8-3: 57544622G/A
8-3: 57654831T/A
8-3: 57711959T/A
8-3: 57712439T/G
8-3: 57737533T/C
8-3: 57791123T/C
8-3: 57827301C/A
8-3: 57854448G/C
8-3: 57955918T/C
8-3: 57967595T/A
8-3: 58031014A/C
8-3: 58213410A/C
8-3: 58309753A/G
8-3: 58369779T/C
8-3: 58403663A/C
8-3: 58425059G/A
8-3: 58481368T/C
8-3: 58519334T/A
8-3: 58593600A/T
8-3: 58625931A/G
8-3: 58663354T/G
8-3: 58663380G/C
8-3: 58663425T/C
8-3: 58671834T/C
8-3: 58671837T/C
8-3: 58671866G/C
8-3: 58798820A/T
8-3: 58870763G/T
8-3: 58942954G/A
8-3: 58978321A/T
8-3: 59118811A/G
8-3: 59144316A/T
8-3: 59267808A/C
8-3: 59471765T/C
8-3: 59483230A/T
8-3: 59578033G/A
8-3: 59628893C/A
8-3: 59660346C/T
8-3: 59677592T/G
8-3: 59788530A/G
8-3: 59836932G/A
8-3: 59919754G/A
8-3: 60099148T/C
8-3: 60177224T/C
8-3: 60179794A/C
8-3: 60240106C/T
8-3: 60278952C/A
8-3: 60347396G/A
8-3: 60467407T/C
8-3: 60511610G/A
8-3: 60563881G/A
8-3: 60632078C/A
8-3: 60650238C/T
8-3: 60660899T/C
8-3: 60749956T/C
8-3: 60795669G/A
8-3: 60951576T/C
8-3: 60979263C/T
8-3: 61048735T/C
8-3: 61099162C/T
8-3: 61179480C/A
8-3: 61187344G/A
8-3: 61187376C/T
8-3: 61209769G/T
8-3: 61336605A/G
8-3: 61367931A/G
8-3: 61391295A/T
8-3: 61489067G/A
8-3: 61548941A/G
8-3: 61580797A/G
8-3: 61607776A/G
8-3: 61684412C/T
8-3: 61764590C/T
8-3: 61811155A/G
8-3: 61870573T/A
8-3: 61961660C/G
8-3: 62008198T/C
8-3: 62049970G/A
8-3: 62140980G/T
8-3: 62157665T/A
8-3: 62182460C/A
8-3: 62272117A/G
8-3: 62357362C/T
8-3: 62426138T/A
8-3: 62525893A/G
8-3: 62567585C/T
8-3: 62654349T/A
8-3: 62687562A/C
8-3: 62724184C/T
8-3: 62899647T/C
8-3: 62901506T/C
8-3: 62949989A/G
8-3: 63047391G/A
8-3: 63052200A/G
8-3: 63052253T/C
8-3: 63070807T/C
8-3: 63094819A/G
8-3: 63095138G/C
8-3: 63095369T/C
8-3: 63150021A/C
8-3: 63184638T/C
8-3: 63228782C/A
8-3: 63229992T/C
8-3: 63258780T/C
8-3: 63260835A/G
8-3: 63277978C/T
8-3: 63319244A/C
8-3: 63330281T/C
8-3: 63348329A/G
8-3: 63367768G/A
8-3: 63380403A/G
8-3: 63410701G/C
8-3: 63426749C/G
8-3: 63427044T/G
8-3: 63427551T/C
8-3: 63446918T/C
8-3: 63487972T/C
8-3: 63569573C/T
8-3: 63600880C/T
8-3: 63631180A/G
8-3: 63698225T/C
8-3: 63711063C/T
8-3: 63724819T/A
8-3: 63727375C/T
8-3: 63754118T/C
8-3: 63785081A/T
8-3: 63800940A/G
8-3: 63819855T/C
8-3: 63828224C/T
8-3: 63828360C/T
8-3: 63833938A/C
8-3: 63855143A/G
8-3: 63863293A/G
8-3: 63874633A/T
8-3: 63885295T/C
8-3: 63896610C/T
8-3: 63897237C/T
8-3: 63914048T/C
8-3: 63916135G/A
8-3: 63924127T/C
8-3: 63944693C/T
8-3: 63954955A/C
8-3: 63966371G/A
8-3: 63979100T/C
8-3: 64034998C/A
8-3: 64054550A/C
8-3: 64069821C/A
8-3: 64111056A/C
8-3: 64162219C/T
8-3: 64199958G/A
8-3: 64216871C/T
8-3: 64340147C/A
8-3: 64349481G/A
8-3: 64360363T/A
8-3: 64387490T/C
8-3: 64436818T/C
8-3: 64463565T/G
8-3: 64540326C/T
8-3: 64617445A/C
8-3: 64647514C/T
8-3: 64734444C/A
8-3: 64761880C/T
8-3: 64801677A/C
8-3: 64854832A/C
8-3: 64926769A/G
8-3: 64988811A/G
8-3: 65042308C/T
8-3: 65193906A/G
8-3: 65324352C/T
8-3: 65341836T/C
8-3: 65370390A/G
8-3: 65432422A/G
8-3: 65437030G/C
8-3: 65443689A/T
8-3: 65473368A/G
8-3: 65500116C/T
8-3: 65538315A/G
8-3: 65566535C/T
8-3: 65609925C/T
8-3: 65624765C/A
8-3: 65650722C/A
8-3: 65693896T/C
8-3: 65758276C/T
8-3: 65812568T/A
8-3: 65867564C/T
8-3: 65973830C/T
8-3: 66060162C/T
8-3: 66126610G/T
8-3: 66162482T/C
8-3: 66235935C/G
8-3: 66362147T/G
8-3: 66476559G/A
8-3: 66488651C/T
8-3: 66536861C/T
8-3: 66560545A/G
8-3: 66572989C/A
8-3: 66584446C/T
8-3: 66596909C/G
8-3: 66681504T/A
8-3: 66698798T/A
8-3: 66754857C/T
8-3: 66878759A/G
8-3: 67064649C/T
8-3: 67077420T/A
8-3: 67317454A/C
8-3: 67344464A/G
8-3: 67358605A/T
8-3: 67388592C/T
8-3: 67428671G/A
8-3: 67573983G/A
8-3: 67762667C/T
8-3: 67841583C/A
8-3: 67963903A/T
8-3: 68137941A/G
8-3: 68294725T/A
8-3: 68383913A/T
8-3: 68557560G/A
8-3: 68721966C/A
8-3: 68933454C/T
8-3: 68933498G/T
8-3: 68933548T/C
8-3: 68933550T/G
8-3: 68933835T/G
8-3: 68935198T/C
8-3: 68975114T/C
8-3: 68978303T/C
8-3: 68979301A/G
8-3: 68980060G/T
8-3: 68980095G/T
8-3: 68985256C/A
8-3: 68985677T/A
8-3: 68985974T/A
8-3: 68989854G/A
8-3: 68990264G/T
8-3: 68990301C/G
8-3: 68990412C/T
8-3: 68990416C/T
8-3: 68993075C/A
8-3: 68993085A/T
8-3: 68993118A/C
8-3: 68993123C/T
8-3: 69226870C/T
8-3: 69386031T/A
8-3: 69426797G/A
8-3: 69628656C/T
8-3: 69652433G/A
8-4: 197445T/A
8-4: 211345A/G
8-4: 212019G/A
8-4: 212313T/A
8-4: 212498C/G
8-4: 213561T/C
8-4: 213958G/A
8-4: 562846C/A
8-4: 742695C/A
8-4: 985684G/T
8-4: 987980C/A
8-4: 1007737A/C
8-4: 1234637T/C
8-4: 1353273G/T
8-4: 1383258C/G
8-4: 1477137A/C
8-4: 1726162T/A
8-4: 1764864G/A
8-4: 1807648C/T
8-4: 1910849T/G
8-4: 2014229C/G
8-4: 2212719C/G
8-4: 2276615G/A
8-4: 2334724G/A
8-4: 2367110A/T
84: 2708622T/A
8-4: 3203084C/T
8-4: 3261134A/G
8-4: 3412950A/G
8-4: 3553274A/G
8-4: 3608288C/T
8-4: 3945034T/A
8-4: 4065012G/C
8-4: 4156967A/T
8-4: 4279145C/T
8-4: 4299089T/A
8-4: 4323662C/T
8-4: 4432895G/A
8-4: 4475830T/C
8-4: 4514071G/C
8-4: 4529099G/A
8-4: 4574792T/G
8-4: 4661210A/G
8-4: 4729279T/C
8-4: 4959002T/C
8-4: 5135029C/G
8-4: 5137223G/A
8-4: 5178414T/C
84: 5196526A/G
8-4: 5244174G/A
8-4: 5276029A/C
8-4: 5300141A/G
8-4: 5330978G/A
8-4: 5370371T/C
8-4: 5382877C/T
8-4: 5424140G/A
8-4: 5425436A/T
8-4: 5425473A/T
8-4: 5425511A/T
8-4: 5471661A/G
8-4: 5471684G/A
8-4: 5511496T/G
8-4: 5559200C/T
8-4: 5605987T/A
8-4: 5616016A/G
8-4: 5616957A/G
8-4: 5638779A/G
8-4: 5728734C/T
8-4: 5732220G/A
8-4: 5825045T/C
8-4: 5826702G/C
8-4: 5946667C/T
8-4: 5952035A/T
8-4: 5966833C/T
8-4: 6045774C/T
8-4: 6192488T/A
8-4: 6288854C/T
8-4: 6399391G/A
8-4: 6457515A/G
8-4: 6466377A/G
8-4: 6468073T/C
8-4: 6502341C/T
8-4: 6520642G/A
8-4: 6566837G/C
8-4: 6619241A/G
8-4: 6745446G/T
8-4: 6761884A/G
84: 6791164G/A
8-4: 6941397A/G
8-4: 7064665G/A
8-4: 7086076G/A
8-4: 7086540A/T
8-4: 7086731T/C
8-4: 7086816G/A
8-4: 7102213C/A
8-4: 7102532G/T
8-4: 7105387A/G
8-4: 7175147G/A
8-4: 7295573G/C
8-4: 7347462A/G
8-4: 7412652T/G
8-4: 7611862C/G
8-4: 7611886C/T
8-4: 7623524C/G
8-4: 7629557C/T
8-4: 7636098C/T
8-4: 7683397A/C
8-4: 7683426C/T
8-4: 7686643T/A
8-4: 7687166T/G
8-4: 7687670G/A
8-4: 7688049T/C
8-4: 7742035C/T
8-4: 7751363C/A
8-4: 7774062A/C
8-4: 7824608A/G
8-4: 7824966C/G
8-4: 7825081A/G
8-4: 7825186G/T
8-4: 7825548A/C
8-4: 7828374G/A
8-4: 7828494C/G
8-4: 7828661G/A
8-4: 7828755G/T
8-4: 7829323A/T
8-4: 7829426C/T
8-4: 7829588C/A
8-4: 7829654T/C
8-4: 7829840G/A
8-4: 7829989T/A
8-4: 7830280G/A
8-4: 7830349T/A
8-4: 7830608C/T
8-4: 7830893C/G
8-4: 7838132A/G
8-4: 7838490C/T
8-4: 7841561A/G
8-4: 7842058A/C
8-4: 7842300G/T
8-4: 7842402T/G
8-4: 7842420T/C
8-4: 7842662C/A
8-4: 7843028G/A
8-4: 7843280A/T
8-4: 7843544A/T
8-4: 7843603A/C
8-4: 7844054A/T
8-4: 7844153A/G
8-4: 7844499G/A
8-4: 7844550G/A
8-4: 7844810T/A
8-4: 7848312T/G
8-4: 7849903C/A
8-4: 7850461G/A
8-4: 7851968C/T
8-4: 7852465G/T
8-4: 7852536T/A
8-4: 7858521G/A
8-4: 7904329C/G
8-4: 7947251A/T
8-4: 8001611A/C
8-4: 8008223A/G
8-4: 8013580C/T
8-4: 8014196T/A
8-4: 8014228A/T
8-4: 8015303A/G
8-4: 8071509G/A
8-4: 8126573A/G
8-4: 8168315G/A
8-4: 8187024G/T
8-4: 8315109T/C
8-4: 8316220G/T
8-4: 8316286G/A
8-4: 8327808G/A
8-4: 8596831G/A
8-4: 8800405A/G
8-4: 8815624T/A
8-4: 8818796T/G
8-4: 8821504A/T
8-4: 8821866T/A
8-4: 8821868G/A
8-4: 8822027C/T
8-4: 8822108G/C
8-4: 8822144T/A
8-4: 8822248G/A
8-4: 8834805T/C
8-4: 8863487G/A
8-4: 8875650T/C
8-4: 8879116C/T
8-4: 8881159T/C
8-4: 8881163A/T
8-4: 8891540T/C
8-4: 8975208C/T
8-4: 8988741G/A
8-4: 9003532T/C
8-4: 9017369G/C
8-4: 9029714T/A
8-4: 9031270T/C
8-4: 9039901G/A
8-4: 9059057C/A
8-4: 9072593A/T
8-4: 9089830T/C
8-4: 9169254T/A
8-4: 9178139G/A
8-4: 9212136T/C
8-4: 9267140C/T
8-4: 9292331C/T
8-4: 9325673G/C
8-4: 9365468T/C
8-4: 9388942G/A
8-4: 9407548T/A
8-4: 9426059C/A
8-4: 9473157A/G
8-4: 9500973C/T
8-4: 9511578G/A
8-4: 9559104A/G
8-4: 9581187A/G
8-4: 9637379A/G
8-4: 9646785G/A
8-4: 9658480A/G
8-4: 9704612A/G
8-4: 9748696G/A
8-4: 9783515A/C
8-4: 9878214G/A
8-4: 10063915G/T
8-4: 10106179C/T
8-4: 10119212T/C
8-4: 10288411T/A
8-4: 10422044C/T
8-4: 10532682G/A
8-4: 10583079G/A
8-4: 10583182T/C
8-4: 10612595T/A
8-4: 10712445G/C
8-4: 10738133A/T
8-4: 10802334G/A
8-4: 10828790G/T
8-4: 10847356G/A
8-4: 10880859G/T
8-4: 11045363G/T
8-4: 11098466G/A
8-4: 11124942G/A
8-4: 11226976A/T
8-4: 11309746C/T
8-4: 11384102C/T
8-4: 11389340A/G
8-4: 11402976C/A
8-4: 11428388A/C
8-4: 11529689C/T
8-4: 11542388A/G
8-4: 11552774T/C
8-4: 11582791G/T
8-4: 11607906A/G
8-4: 11636194C/T
8-4: 11709295G/T
8-4: 11759943T/C
8-4: 11800361G/A
8-4: 11827220G/A
8-4: 11900828A/G
8-4: 11953565A/G
8-4: 11995862G/A
8-4: 12122650G/A
8-4: 12170960A/G
8-4: 12217271A/C
8-4: 12233959T/G
8-4: 12234585A/C
8-4: 12249709T/C
8-4: 12250373A/G
8-4: 12263611G/T
8-4: 12334170T/A
8-4: 12399647C/G
8-4: 12455256T/C
8-4: 12517321G/T
8-4: 12521173A/G
8-4: 12605317A/C
8-4: 12622928G/A
8-4: 12643637T/C
8-4: 12644258C/A
8-4: 12652398A/C
8-4: 12681478C/T
8-4: 12682513A/G
8-4: 12684944G/A
8-4: 12772197C/T
8-4: 12876275G/A
8-4: 12904826G/T
8-4: 12935056T/A
8-4: 12981061G/C
8-4: 13006826G/A
8-4: 13060410C/T
8-4: 13079614G/C
8-4: 13120347G/A
8-4: 13142008C/G
8-4: 13162179A/T
8-4: 13187349A/G
8-4: 13238000T/C
8-4: 13293704T/A
8-4: 13323114C/G
8-4: 13332600C/T
8-4: 13388479G/A
8-4: 13413928A/G
8-4: 13454657C/T
8-4: 13498041G/A
8-4: 13550967G/T
8-4: 13701549T/C
8-4: 13751261A/G
8-4: 13828953G/T
8-4: 13854620A/T
8-4: 13888034C/T
8-4: 13998660C/T
8-4: 14007196A/T
8-4: 14007457A/G
8-4: 14008942A/T
8-4: 14041827T/C
8-4: 14082105C/T
8-4: 14131907T/C
8-4: 14146286A/G
8-4: 14161380C/T
8-4: 14223607A/G
8-4: 14310521C/T
8-4: 14464313C/G
8-4: 14472319T/C
8-4: 14533735T/A
8-4: 14550844T/A
8-4: 14581719A/G
8-4: 14604380T/G
8-4: 14604519A/C
8-4: 14612082G/A
8-4: 14625047T/G
8-4: 14627027T/C
8-4: 14655187A/C
8-4: 14655352T/A
8-4: 14667200A/C
8-4: 14779076A/T
8-4: 14781255A/G
8-4: 14791462T/A
8-4: 14792485T/A
8-4: 14856985G/A
8-4: 14939721T/A
8-4: 14984010A/G
8-4: 14991319A/G
8-4: 15033569C/T
8-4: 15072997A/C
8-4: 15137028G/C
8-4: 15166731T/C
8-4: 15266550T/C
8-4: 15268849C/A
8-4: 15313326A/T
8-4: 15348733T/C
8-4: 15377800G/T
8-4: 15418615G/A
8-4: 15457771C/T
8-4: 15514429G/A
8-4: 15562304G/A
8-4: 15567314T/G
8-4: 15567784G/A
84: 15578678C/A
8-4: 15618538A/G
8-4: 15629232C/G
8-4: 15647693T/A
8-4: 15662112T/A
8-4: 15677968T/C
8-4: 15694918C/T
8-4: 15715811A/G
8-4: 15791567G/T
8-4: 15839835T/G
8-4: 15850297T/A
8-4: 15864967T/C
8-4: 15875305A/T
8-4: 15905934T/A
8-4: 15954560A/G
8-4: 16006495C/T
8-4: 16043117T/C
8-4: 16055454A/T
8-4: 16094840G/A
8-4: 16124960T/G
8-4: 16196474A/G
8-4: 16240750T/C
8-4: 16286884G/A
8-4: 16317992T/C
8-4: 16398208A/C
8-4: 16408824C/G
8-4: 16431821G/A
8-4: 16472568A/G
8-4: 16514421A/C
8-4: 16598952C/T
8-4: 16672387G/A
8-4: 16699354G/C
8-4: 16736408T/C
8-4: 16788139T/C
8-4: 16829316A/C
8-4: 16862021A/G
8-4: 16864762A/G
8-4: 16865077G/T
8-4: 16883077T/C
84: 16911731G/A
8-4: 16938337G/A
8-4: 16997726T/C
8-4: 17021099G/A
8-4: 17025689A/T
8-4: 17026672T/C
8-4: 17026976A/G
8-4: 17036781T/C
8-4: 17038247G/A
8-4: 17070002G/A
8-4: 17070366C/T
8-4: 17129996C/T
8-4: 17162152C/T
8-4: 17192797G/A
8-4: 17220439G/C
8-4: 17254136G/A
8-4: 17326881T/C
8-4: 17416685T/C
8-4: 17420773C/T
8-4: 17449693G/A
8-4: 17504424T/C
8-4: 17531871A/C
8-4: 17591583C/G
8-4: 17740991T/A
8-4: 17751426A/G
8-4: 17762988C/G
8-4: 17860583T/G
8-4: 17873729T/A
8-4: 17935830A/T
8-4: 17951205A/C
8-4: 17962439T/C
8-4: 17975768G/A
84: 17988016T/G
8-4: 18018332G/A
8-4: 18051481C/G
8-4: 18083589A/G
8-4: 18117680C/T
8-4: 18123171A/G
8-4: 18170747C/G
8-4: 18208668G/A
8-4: 18220046C/T
8-4: 18290203T/A
8-4: 18301370T/C
8-4: 18335509T/C
8-4: 18371595G/A
8-4: 18384543G/A
8-4: 18398167T/C
8-4: 18400686G/T
8-4: 18420685T/C
8-4: 18437698C/G
8-4: 18441445A/G
8-4: 18449673G/T
8-4: 18457052C/G
8-4: 18461485A/T
8-4: 18491372C/T
8-4: 18523712A/G
8-4: 18534894G/A
8-4: 18603035G/A
8-4: 18605413G/T
8-4: 18616004C/T
8-4: 18624106C/T
8-4: 18644461T/C
8-4: 18659419G/A
8-4: 18681223T/A
8-4: 18681349C/T
8-4: 18681400C/T
8-4: 18707867C/G
8-4: 18748327C/T
8-4: 18750259T/C
8-4: 18784797A/T
8-4: 18827163A/G
8-4: 18940175A/G
8-4: 19014955G/C
8-4: 19048951C/T
8-4: 19063429G/A
8-4: 19098446A/G
8-4: 19108680G/A
8-4: 19148687C/T
8-4: 19161605G/A
8-4: 19166657A/T
8-4: 19168217A/G
8-4: 19168727A/G
8-4: 19168975A/T
8-4: 19169120G/A
8-4: 19169415C/T
8-4: 19173097C/T
8-4: 19173899C/T
8-4: 19173956A/G
8-4: 19174159A/G
8-4: 19174489G/C
8-4: 19174507T/A
8-4: 19175024G/A
8-4: 19177134A/G
8-4: 19177165C/G
8-4: 19177616C/T
8-4: 19177667T/A
8-4: 19177886T/C
8-4: 19179479G/A
8-4: 19179692T/A
8-4: 19179880A/G
8-4: 19181372T/C
8-4: 19182518A/C
8-4: 19184402G/A
8-4: 19184752A/G
8-4: 19200180T/G
8-4: 19210688T/A
8-4: 19243963G/A
8-4: 19271861C/T
8-4: 19311808G/A
8-4: 19327839T/A
8-4: 19413821C/T
8-4: 19413980G/T
8-4: 19480382T/C
84: 19645715G/A
8-4: 19773820A/G
8-4: 19792910A/C
8-4: 19823853G/A
8-4: 19860494G/A
8-4: 19920919T/A
8-4: 19932688A/G
8-4: 20090510T/C
8-4: 20099506A/G
8-4: 20122641T/A
8-4: 20187983T/C
8-4: 20195511A/C
8-4: 20205136C/A
8-4: 20205525G/T
8-4: 20206228A/T
8-4: 20207166A/G
8-4: 20207330C/T
8-4: 20232294A/G
8-4: 20257870G/A
8-4: 20272834T/C
8-4: 20278051G/A
8-4: 20304225A/G
8-4: 20323507A/T
8-4: 20358519T/C
8-4: 20365705T/C
8-4: 20376610G/A
8-4: 20381764C/T
8-4: 20402465A/G
8-4: 20467695T/C
8-4: 20507307C/T
8-4: 20530096T/C
8-4: 20561433G/A
8-4: 20593606T/C
8-4: 20657976T/G
8-4: 20690560A/T
8-4: 20736339T/A
8-4: 20747144G/T
8-4: 20749319T/C
8-4: 20763352G/A
8-4: 20797000A/G
8-4: 20801316T/C
8-4: 20899043C/T
8-4: 21143377C/T
8-4: 21237445T/G
8-4: 21420618C/T
8-4: 22121504G/A
8-4: 22184839A/T
8-4: 22230300T/C
8-4: 22320330G/A
8-4: 22431038T/C
8-4: 22432964G/A
8-4: 22443081G/A
8-4: 22450332G/T
8-4: 22469560C/A
8-4: 22488831T/C
8-4: 22491328C/T
8-4: 22503094A/G
8-4: 22546343G/A
8-4: 22564968C/T
8-4: 22656183G/A
8-4: 22685400A/C
8-4: 22705132T/G
8-4: 22728448G/A
8-4: 22746811C/A
8-4: 22811612C/T
8-4: 22893583G/A
8-4: 22912292C/T
8-4: 22922316A/G
8-4: 22955168T/A
8-4: 22989500G/A
8-4: 23006009A/T
8-4: 23021632C/T
8-4: 23101515G/A
8-4: 23355856T/A
8-4: 23411556T/C
8-4: 23424968A/G
8-4: 23430191G/A
8-4: 23465713G/A
8-4: 23494637C/T
8-4: 23495021G/A
8-4: 23533372G/T
8-4: 23645529C/A
8-4: 23679434A/G
8-4: 23690065C/T
8-4: 23695805G/C
8-4: 23716946A/G
8-4: 23762801A/G
8-4: 23790804C/T
8-4: 23802773T/G
8-4: 23841023A/G
8-4: 23855517C/T
8-4: 23903349G/T
8-4: 23903739G/A
8-4: 23919906C/T
8-4: 23947237C/T
8-4: 24011186C/G
8-4: 24095732C/T
8-4: 24109664G/A
8-4: 24111852G/A
8-4: 24134262G/T
8-4: 24138463A/T
8-4: 24139022C/T
8-4: 24188144A/G
8-4: 24253640C/T
8-4: 24299755C/T
8-4: 24348999T/C
8-4: 24389814G/A
8-4: 24417866C/A
8-4: 24418075G/A
8-4: 24418241G/A
8-4: 24435019C/A
8-4: 24445498G/A
8-4: 24456543T/A
8-4: 24533332C/G
8-4: 24545378C/T
8-4: 24579770G/A
8-4: 24595426A/C
8-4: 24595497A/T
8-4: 24595603C/T
8-4: 24621713T/G
8-4: 24710568G/T
8-4: 24739084A/G
8-4: 24775394T/C
8-4: 24804920T/C
8-4: 24840980A/G
8-4: 24895746G/A
8-4: 24941837A/G
8-4: 24983303T/C
8-4: 25015977G/A
8-4: 25042347G/A
8-4: 25058636C/T
8-4: 25085809A/G
8-4: 25141057A/G
8-4: 25221345G/A
8-4: 25257582T/C
8-4: 25384624T/A
8-4: 25386182A/C
8-4: 25415729C/T
8-4: 25486472G/T
8-4: 25514111C/T
8-4: 25550211T/G
8-4: 25560146T/C
8-4: 25570979C/T
8-4: 25590303G/C
8-4: 25600444C/T
8-4: 25624296G/A
8-4: 25659932C/T
8-4: 25680792T/C
8-4: 25704753G/A
8-4: 25929365T/C
8-4: 26019611T/G
8-4: 26058295G/A
8-4: 26127819T/G
8-4: 26161657T/C
8-4: 26184926T/A
8-4: 26200057C/A
8-4: 26200266C/G
8-4: 26201824A/T
8-4: 26201948G/A
8-4: 26202920G/T
8-4: 26203016C/A
8-4: 26203233T/C
8-4: 26203598G/A
8-4: 26204011G/T
8-4: 26216517G/A
8-4: 26217471C/A
8-4: 26218112T/C
8-4: 26229802T/C
8-4: 26246428A/T
8-4: 26276993G/A
8-4: 26312264T/A
8-4: 26401094C/T
8-4: 26434404T/A
8-4: 26434681C/T
8-4: 26452156T/C
8-4: 26526037T/G
8-4: 26547191A/G
8-4: 26612030T/A
8-4: 26664079A/G
8-4: 26710743C/T
8-4: 26776054A/G
8-4: 26871676T/C
8-4: 26909472G/C
8-4: 26968712T/G
8-4: 26998975G/A
8-4: 26999851A/.
8-4: 26999852G/.
8-4: 27002002T/C
8-4: 27066512A/G
8-4: 27067549T/A
8-4: 27099435G/T
8-4: 27124786G/A
8-4: 27224037G/C
8-4: 27340766C/T
8-4: 27438611C/G
8-4: 27469086A/C
8-4: 27483720T/C
8-4: 27511142A/T
8-4: 27525070G/A
8-4: 27525175A/G
8-4: 27542078T/A
8-4: 27581530A/G
8-4: 27607726G/C
8-4: 27630461A/T
8-4: 27630655T/C
8-4: 27640777T/C
8-4: 27671273G/C
8-4: 27691203T/G
8-4: 27699327C/A
8-4: 27699530G/A
8-4: 27749396A/G
8-4: 27856893C/G
8-4: 27858102G/C
8-4: 27887066A/G
8-4: 27892591A/C
8-4: 27914374T/C
8-4: 27999059G/A
8-4: 28001435A/C
8-4: 28023499T/A
8-4: 28097202T/A
8-4: 28134393A/G
8-4: 28267587G/A
8-4: 28324586A/T
8-4: 28514713G/A
8-4: 28552104C/G
8-4: 28688673G/T
8-4: 28700362T/A
8-4: 28700517C/T
8-4: 28717434G/A
8-4: 28731649T/A
8-4: 28753988G/A
8-4: 28789281G/A
8-4: 28799825C/A
8-4: 28830903G/A
8-4: 28918822G/A
8-4: 28971355G/C
8-4: 29070715G/T
8-4: 29113177G/A
8-4: 29123233G/A
8-4: 29236446C/T
8-4: 29259023A/G
8-4: 29262425G/A
8-4: 29263059G/T
8-4: 29263568T/A
8-4: 29269486G/A
8-4: 29269607C/A
8-4: 29269813T/A
8-4: 29314209G/A
8-4: 29358996C/T
8-4: 29372771A/G
8-4: 29375733C/T
8-4: 29388721G/T
8-4: 29443601A/G
8-4: 29476519C/T
8-4: 29523399C/T
8-4: 29541601C/T
8-4: 29598421A/T
8-4: 29642745C/T
8-4: 29674149A/G
8-4: 29714175A/T
8-4: 29732179A/C
8-4: 29796616G/A
8-4: 29917927C/T
8-4: 30067290C/T
8-4: 30115722A/G
8-4: 30125850T/C
8-4: 30151705G/A
8-4: 30154948T/G
8-4: 30165111C/A
8-4: 30203527G/C
8-4: 30218782T/G
8-4: 30229217T/C
8-4: 30242366C/A
8-4: 30261227G/T
8-4: 30273726C/T
8-4: 30299012T/A
8-4: 30342567G/A
8-4: 30375896A/G
8-4: 30491428A/T
8-4: 30682490G/A
8-4: 30692726C/T
8-4: 30721200G/T
8-4: 30724147A/C
8-4: 30819862G/A
8-4: 30857311A/G
8-4: 30871489A/G
8-4: 30914429C/T
8-4: 30941417G/T
8-4: 30980242A/C
8-4: 31038939T/G
8-4: 31038949G/A
8-4: 31038953C/T
8-4: 31038983A/G
8-4: 31039022G/A
8-4: 31039127A/G
8-4: 31039157A/.
8-4: 31043100C/T
8-4: 31120100C/T
8-4: 31172131A/G
8-4: 31203757G/T
8-4: 31227102C/A
8-4: 31237236A/G
8-4: 31237545G/A
8-4: 31297860G/A
8-4: 31354134T/G
8-4: 31516447C/A
8-4: 31525718C/T
8-4: 31543040A/C
8-4: 31553236A/T
8-4: 31556393A/G
8-4: 31584523C/T
8-4: 31645188A/T
8-4: 31689427G/A
8-4: 31706401C/T
8-4: 31759337A/G
8-4: 31775856C/T
8-4: 31792725C/T
8-4: 31833444A/G
8-4: 31834578G/A
8-4: 31835014A/C
8-4: 31835177T/C
8-4: 31858627C/T
8-4: 31882715G/A
8-4: 31896723G/A
8-4: 31965293C/T
8-4: 31965302T/G
8-4: 31995844A/T
8-4: 32075415G/A
8-4: 32115125C/T
8-4: 32134389C/T
8-4: 32188554A/G
8-4: 32264501G/A
8-4: 32280387C/T
8-4: 32311949A/G
8-4: 32372678A/T
8-4: 32447157T/C
8-4: 32563576C/A
8-4: 32581371A/T
8-4: 32625828T/C
8-4: 32626181G/A
8-4: 32626183C/T
8-4: 32626200A/T
8-4: 32831216G/A
8-4: 32934460G/A
8-4: 32987154G/C
8-4: 33012539C/T
8-4: 33091808C/G
8-4: 33113316A/T
8-4: 33140939A/T
8-4: 33311061G/C
8-4: 33353088A/G
8-4: 33358470G/A
8-4: 33378996C/T
8-4: 33455640G/A
8-4: 33456749G/A
8-4: 33456838A/C
8-4: 33459199A/C
8-4: 33467088A/T
8-4: 33467851A/C
8-4: 33487423G/A
8-4: 33490446G/C
8-4: 33526828T/C
8-4: 33566823A/T
8-4: 33591594C/T
8-4: 33623785C/T
8-4: 33741535G/A
8-4: 33991347C/T
8-4: 34055460C/T
8-4: 34092637A/G
8-4: 34107400C/T
8-4: 34107531A/T
8-4: 34147117A/T
8-4: 34262276C/T
8-4: 34446814T/A
8-4: 34457559A/G
8-4: 34471571A/T
8-4: 34527617A/G
8-4: 34596855G/A
8-4: 34667040C/A
8-4: 34696231T/G
8-4: 34730018T/C
8-4: 34793665C/A
8-4: 34930648G/A
8-4: 34934751A/G
8-4: 34935259A/G
8-4: 34935269T/A
8-4: 34935279C/G
8-4: 34935342T/A
8-4: 34952568A/C
8-4: 34978124G/A
8-4: 35029100T/A
8-4: 35058139A/G
8-4: 35067904A/G
8-4: 35067949T/A
8-4: 35072718G/A
8-4: 35083910T/C
8-4: 35084534G/A
8-4: 35102945C/T
8-4: 35256755A/G
8-4: 35273691A/G
8-4: 35302533C/A
8-4: 35316947T/A
8-4: 35408621C/T
8-4: 35490992A/T
8-4: 35674667A/G
8-4: 35675635T/C
8-4: 35822199T/C
8-4: 35957777C/T
8-4: 36015472A/G
8-4: 36049044A/G
8-4: 36090965G/A
8-4: 36116337C/T
8-4: 36141784C/T
8-4: 36171769T/A
8-4: 36241915C/A
8-4: 36310922A/G
8-4: 36335121C/T
8-4: 36415008G/A
8-4: 36551525A/G
8-4: 36565031A/G
8-4: 36628059G/A
8-4: 36670313T/C
84: 36773908G/A
8-4: 36809387A/T
8-4: 36846395T/C
8-4: 36900810C/A
8-4: 36954674T/A
8-4: 37102841A/G
8-4: 37120425C/A
8-4: 37215083C/T
8-4: 37279804G/T
8-4: 37330358G/A
8-4: 37333681A/G
8-4: 37333692T/C
8-4: 37345956T/C
8-4: 37422296T/A
8-4: 37450033T/G
8-4: 37501443C/T
8-4: 37501454A/G
8-4: 37501472C/T
8-4: 37501478A/T
8-4: 37501483A/T
8-4: 37501488G/A
8-4: 37501525T/A
8-4: 37501553C/T
8-4: 37502525G/T
8-4: 37502744G/A
8-4: 37504145G/T
8-4: 37566439G/A
8-4: 37655462A/T
8-4: 37668530T/A
8-4: 37669465C/T
8-4: 37674691C/T
8-4: 37681909A/C
8-4: 37682708A/.
8-4: 37689370T/C
8-4: 37689481T/A
8-4: 37689656C/T
8-4: 37709419C/A
8-4: 37711119C/A
8-4: 37711143A/G
8-4: 37711382A/G
8-4: 37711496G/T
8-4: 37730847T/C
8-4: 37756322G/A
8-4: 37775053T/C
8-4: 37798303T/C
8-4: 37845236A/G
8-4: 37912144C/A
8-4: 37966526A/G
8-4: 37990834G/A
8-4: 38006235T/C
8-4: 38037252C/T
8-4: 38065505C/T
8-4: 38118589A/C
8-4: 38371462G/A
8-4: 38399881T/C
8-4: 38427506T/A
8-4: 38492141A/G
8-4: 38531441G/A
8-4: 38642464T/C
8-4: 38646786T/C
8-4: 38648330A/T
8-4: 38664869G/T
8-4: 38691741A/G
8-4: 38701882T/A
8-4: 38775089C/T
8-4: 38861120C/T
8-4: 38944729T/C
8-4: 39079090C/T
8-4: 39170018C/A
8-4: 39190263C/A
8-4: 39207825T/C
8-4: 39212299G/A
8-4: 39214915T/C
8-4: 39296503T/C
8-4: 39298963T/C
8-4: 39310345A/G
8-4: 39399310C/A
8-4: 39497168C/T
8-4: 39513974A/G
8-4: 39530213G/A
8-4: 39579712T/C
8-4: 39613898T/C
8-4: 40010606C/T
8-4: 40318985A/C
8-4: 40335114T/G
8-4: 40336935G/A
8-4: 40340105C/T
8-4: 40361465C/A
8-4: 40395572T/G
8-4: 40405684C/T
8-4: 40559469A/C
8-4: 40614402C/A
8-4: 40717143C/T
8-4: 40719018C/T
8-4: 40750426C/T
8-4: 40774480A/G
8-4: 40948367G/T
8-4: 41139892A/G
8-4: 41158272C/A
8-4: 41158671T/.
8-4: 41159624C/G
8-4: 41351874G/A
8-4: 41662379C/G
8-4: 41666207A/G
8-4: 41993835A/T
8-4: 42143029C/T
8-4: 42381919A/C
8-4: 42408135A/C
8-4: 42459025G/A
8-4: 42518771G/C
8-4: 42843438A/G
8-4: 42865115T/A
8-4: 42878275T/C
8-4: 42886898A/C
8-4: 42897900G/C
8-4: 42926043C/T
8-4: 42956113T/C
8-4: 43049947T/C
8-4: 43093705T/C
8-4: 43173407T/G
8-4: 43431802C/A
8-4: 43445967G/A
8-4: 43489172A/T
8-4: 43541507T/C
8-4: 43578568A/G
8-4: 43651996T/C
8-4: 43698249C/T
8-4: 43712976T/G
8-4: 43724728C/A
8-4: 43744968T/G
8-4: 43825836C/T
8-4: 43845344T/A
8-4: 44228506C/T
8-4: 44266543G/A
8-4: 44374396A/T
8-4: 44405565A/T
8-4: 44454887G/A
8-4: 44487336C/G
8-4: 44614614G/T
8-4: 44865493C/A
8-4: 44885236A/C
8-4: 44915870A/T
8-4: 44962285A/C
8-4: 45028602C/T
8-4: 45056036G/C
8-4: 45061306C/T
8-4: 45164718G/T
8-4: 45209918A/G
8-4: 45286695C/T
8-4: 45435243C/T
8-4: 45440969A/G
8-4: 45471116C/T
8-4: 45503118G/C
8-4: 45536754A/T
8-4: 45555929C/T
8-4: 45589526A/C
8-4: 45660096C/T
8-4: 45761425C/A
8-4: 45820033T/C
8-4: 45882725G/T
8-4: 45890968G/A
8-4: 46034684T/A
8-4: 46088631T/C
8-4: 46125507T/G
8-4: 46168200T/C
8-4: 46177825T/C
8-4: 46230180T/A
8-4: 46253052C/G
8-4: 46389442G/C
8-4: 46409391T/C
8-4: 46436247T/C
8-4: 46522498A/C
8-4: 46544320A/T
8-4: 46564166G/A
8-4: 46580544C/A
8-4: 46640655C/T
8-4: 46828788T/A
8-4: 46832773A/G
8-4: 46855723G/A
8-4: 46963122T/C
8-4: 46999665A/G
8-4: 47013697A/C
8-4: 47121432T/A
8-4: 47186013A/G
8-4: 47345507G/A
8-4: 47460174A/G
8-4: 47499713C/A
8-4: 47663252G/T
8-4: 47821036G/A
8-4: 47881896A/G
8-4: 47923120C/T
8-4: 48168131A/G
8-4: 48246662A/G
8-4: 48422898G/A
8-4: 48512074T/G
8-4: 48551693A/G
8-4: 48562356T/C
8-4: 48663696C/T
8-4: 48835335A/G
8-4: 48836694G/A
8-4: 48897565T/G
8-4: 49054510C/T
8-4: 49061870A/T
8-4: 49093491A/G
8-4: 49123881A/C
8-4: 49152291T/G
8-4: 49169255T/C
8-4: 49194140C/T
8-4: 49327551A/T
8-4: 49503186T/C
8-4: 49560464A/G
8-4: 49595386C/T
8-4: 49604723C/T
8-4: 49622099T/C
8-4: 49665096C/T
8-4: 49735225C/T
8-4: 49746897A/T
8-4: 49976338A/T
8-4: 50037387A/G
8-4: 50150863G/T
8-4: 50244624T/C
8-4: 50284622A/G
8-4: 50470754G/C
8-4: 50508383A/T
8-4: 50590522A/T
8-4: 50618578T/C
8-4: 50710725T/A
8-4: 50794378C/A
8-4: 50884084C/A
8-4: 51023638T/A
8-4: 51040477C/T
8-4: 51061904G/A
8-4: 51317484A/G
8-4: 51375449C/T
8-4: 51476785T/C
8-4: 51533178C/T
8-4: 51581630T/G
8-4: 51731771T/C
8-4: 51942122C/G
8-4: 52087001C/T
8-4: 52111083C/A
8-4: 52261851A/G
8-4: 52453227T/C
8-4: 52517601C/T
8-4: 52519237T/C
8-4: 52544363C/T
8-4: 52608689A/T
8-4: 52626317G/T
8-4: 52626773C/G
8-4: 52706809G/C
8-4: 52742035C/G
8-4: 52746054G/A
8-4: 52746134G/A
8-4: 52754133T/C
8-4: 52812377C/G
8-4: 52818715A/G
8-4: 52818776A/G
8-4: 52819236A/T
8-4: 52819247T/C
8-4: 52819309A/G
8-4: 52819584T/A
8-4: 52819686G/A
8-4: 52819755A/G
8-4: 52819760T/C
8-4: 52819821T/G
8-4: 52821562C/T
8-4: 52821875G/A
8-4: 52821998G/A
8-4: 52822051A/G
8-4: 52822101C/A
8-4: 52822203G/A
8-4: 52822309T/A
8-4: 52822417A/T
8-4: 52822591C/G
8-4: 52822628C/T
8-4: 52823057C/T
8-4: 52823325C/T
8-4: 52823328T/C
8-4: 52823780T/C
8-4: 52823918C/T
8-4: 52823965A/G
8-4: 52833324G/T
8-4: 52844788A/G
8-4: 52864589T/C
8-4: 52884707T/G
8-4: 52901662A/G
8-4: 52935785G/C
8-4: 52961549T/C
8-4: 52975964A/G
8-4: 52987670A/T
8-4: 53015734T/A
8-4: 53103859G/A
8-4: 53178843T/C
8-4: 53207963A/T
8-4: 53310269T/A
8-4: 53315650G/A
8-4: 53367729A/G
8-4: 53549104T/C
8-4: 53655567C/T
8-4: 53781644G/A
8-4: 53880658C/T
8-4: 54180503G/T
8-4: 54200543A/T
8-4: 54288924C/A
8-4: 54317741A/G
8-4: 54404489T/C
8-4: 54448931C/A
8-4: 54564734A/G
8-4: 54676895A/G
8-4: 54693352A/C
8-4: 54877272A/G
8-4: 54881071A/T
8-4: 55009864C/T
8-4: 55045281A/G
8-4: 55236963A/G
8-4: 55463632T/C
8-4: 55519674C/A
8-4: 55540773G/A
8-4: 56150350T/C
1-1: 3330429A/T
1-1: 5871150C/T
1-1: 13762908C/T
1-1: 22606239C/T
1-1: 25073941G/C
1-1: 25882429C/T
1-1: 28855478G/T
1-1: 33311929A/T
1-1: 34286924G/A
1-1: 35559224A/G
1-1: 42894456A/T
1-1: 45554168T/G
1-1: 45587954T/A
1-1: 48120625T/C
1-1: 53554447A/G
1-1: 60221893A/G
1-1: 68562093T/A
1-1: 78496661C/T
1-2: 15050806T/C
1-2: 19590226G/A
1-2: 21550720T/G
1-2: 23353030C/A
1-2: 29301666C/T
1-2: 29370801C/A
1-2: 29777184C/T
1-2: 31963597T/C
1-2: 38816057A/C
1-2: 42125809G/A
1-2: 43446321A/G
1-2: 49013367A/C
1-2: 50918193A/G
1-2: 63261570G/T
1-3: 1914789G/T
1-3: 3427213A/G
1-3: 4019979T/C
1-3: 8131313A/T
1-3: 9376962A/T
1-3: 11146707G/A
1-3: 13000892G/A
1-3: 16054123T/C
1-3: 17466569C/T
1-3: 17609958C/T
1-3: 22390306G/A
1-3: 23961829T/A
1-3: 24716713T/C
1-3: 25922304C/A
1-3: 26190206T/C
1-3: 30407276G/T
1-3: 34760460G/A
1-3: 34770376T/C
1-3: 38538073C/T
1-3: 48728338G/A
1-3: 48884035T/A
1-3: 51488963T/A
1-3: 52170209G/A
1-3: 55752142G/T
1-3: 59759855C/T
1-3: 61363532T/A
1-3: 61592892C/A
1-3: 66639165A/T
1-3: 72129060T/G
1-4: 11907087T/G
1-4: 12946127C/A
1-4: 14284924A/T
1-4: 15437771T/A
1-4: 17651879C/A
1-4: 18102557G/C
1-4: 19044815T/C
1-4: 19375283T/A
1-4: 19983620C/A
1-4: 23363178C/T
1-4: 25655864G/T
1-4: 26608661G/A
1-4: 27569245A/C
1-4: 27605342T/C
1-4: 28662185A/G
1-4: 29739263A/C
1-4: 31075738C/T
1-4: 36425166G/T
1-4: 38672130G/A
1-4: 40941244A/T
1-4: 40995565C/A
1-4: 42034881T/C
1-4: 42116403G/C
1-4: 42165029C/T
1-4: 43498078T/C
1-4: 47819013A/G
1-4: 48090516C/A
1-4: 52828088A/G
1-4: 57246923G/A
1-4: 71029774T/C
1-4: 74838726T/C
2-1: 5360590A/G
2-1: 10315447C/T
2-1: 12816408A/C
2-1: 22658464T/C
2-1: 22758449A/C
2-1: 25777944T/C
2-1: 29714337A/C
2-1: 29762115G/A
2-1: 31433646C/T
2-1: 36200056A/T
2-1: 42637134G/A
2-1: 42874383C/T
2-1: 44248706A/G
2-1: 46292152A/C
2-1: 53422409G/T
2-1: 53641877T/C
2-1: 54633217T/C
2-1: 55315354G/A
2-1: 58962607A/G
2-1: 66535756C/A
2-1: 66599611G/A
2-1: 76907968G/A
2-2: 16857642T/A
2-2: 20241171A/C
2-2: 20283506C/T
2-2: 20595859G/C
2-2: 24572754C/T
2-2: 27849543A/C
2-2: 29328232G/A
2-2: 33228998G/T
2-2: 41448468C/A
2-2: 47341918T/C
2-2: 57912068A/G
2-3: 7770443T/C
2-3: 13829399C/T
2-3: 15363415A/C
2-3: 22751704G/A
2-3: 26590913T/G
2-3: 27402866C/G
2-3: 33681635A/C
2-3: 36108330T/G
2-3: 38591720C/G
2-3: 39781786T/A
2-3: 40214837G/A
2-3: 40459510G/A
2-3: 40584801T/C
2-3: 40637023A/G
2-3: 44973439A/T
2-3: 45661612A/T
2-3: 50591490G/A
2-3: 51889613C/T
2-3: 52004003A/G
2-3: 54381495T/C
2-3: 57563392T/A
2-4: 8373089T/C
2-4: 14728546G/T
2-4: 15441290A/G
2-4: 19029841A/T
2-4: 20943338G/C
2-4: 23029574G/A
2-4: 24390888T/C
2-4: 26028224G/A
2-4: 35132296A/T
2-4: 38041892C/G
2-4: 42386532G/A
2-4: 42993507C/G
2-4: 48067780G/A
2-4: 53355762T/C
2-4: 53800160C/T
2-4: 54549498G/A
2-4: 59199328A/G
2-4: 59219092C/G
3-1: 6034576C/A
3-1: 10912409A/C
3-1: 10980231A/T
3-1: 12107368T/C
3-1: 13034558A/G
3-1: 16284506C/T
3-1: 17706753A/T
3-1: 19899508A/T
3-1: 24120257C/A
3-1: 25814470G/C
3-1: 36460224C/T
3-1: 39471765C/T
3-1: 41184442A/G
3-1: 46369034G/A
3-1: 47093168G/T
3-1: 49851779T/G
3-1: 56502858G/A
3-1: 59027455A/G
3-1: 59798350C/T
3-1: 59949951T/G
3-1: 63317675C/A
3-1: 63734951A/T
3-1: 63865278A/G
3-1: 64011339A/T
3-1: 65815805T/C
3-1: 66625823A/T
3-1: 66663824C/G
3-1: 75600149A/G
3-1: 75811639T/A
3-1: 80204895A/C
3-1: 88469470C/A
3-1: 91976177A/T
3-2: 1671450T/G
3-2: 7226214A/T
3-2: 8825281G/T
3-2: 9829566T/A
3-2: 10944087T/A
3-2: 23638244C/G
3-2: 24747068T/A
3-2: 25477677T/C
3-2: 29066141T/G
3-2: 34905348T/A
3-2: 39605714G/T
3-2: 50485146A/T
3-2: 51253474G/A
3-2: 51585104A/T
3-2: 52445300C/G
3-2: 55347493C/A
3-2: 56456971G/C
3-2: 58957278C/G
3-2: 60587993T/G
3-2: 66380417T/C
3-2: 68755047A/C
3-2: 73066864A/G
3-2: 85131834G/A
3-3: 9316149A/G
3-3: 10181532A/G
3-3: 14722302T/C
3-3: 16671432G/C
3-3: 19188094T/A
3-3: 20126184T/G
3-3: 22229867G/A
3-3: 31000851A/G
3-3: 37248092T/C
3-3: 44020444G/A
3-3: 44401146G/A
3-3: 46746991T/C
3-3: 47177491T/G
3-3: 47628893A/G
3-3: 48054820C/T
3-3: 51677083T/C
3-3: 52015650G/A
3-3: 53584680A/C
3-3: 55661668C/T
3-3: 57383820A/G
3-3: 59573394T/A
3-3: 60961724G/C
3-3: 64765518A/G
3-3: 72863042T/C
3-3: 72991457T/C
3-3: 78021896T/A
3-4: 7379567T/C
34: 9663397C/T
3-4: 15669758G/A
3-4: 19219627C/G
3-4: 20754438C/A
3-4: 21817913T/C
3-4: 23876057C/G
3-4: 27959211G/A
3-4: 34842660C/A
3-4: 40414843A/G
3-4: 45030965A/G
3-4: 46023171G/A
3-4: 46604984C/T
3-4: 48883901A/C
3-4: 49713339A/C
3-4: 51255426A/T
3-4: 51605550C/A
3-4: 53841097G/T
4-1: 6551058T/A
4-1: 15550746G/C
4-1: 28140869C/T
4-1: 29347214C/T
4-1: 31520852A/G
4-1: 31644794C/T
4-1: 33956934A/T
4-1: 37365408T/G
4-1: 37657711G/T
4-1: 38566447A/G
4-1: 39085949C/G
4-1: 39191067T/A
4-1: 43661649G/T
4-1: 43864730A/T
4-1: 45006016A/G
4-1: 45149457T/G
4-1: 49831122A/G
4-1: 55509200C/T
4-1: 59391562G/A
4-1: 66540047T/C
4-1: 71395662G/A
4-1: 75702743G/A
4-1: 75773809A/G
4-1: 75793241T/C
4-1: 77379496G/A
4-1: 77406134A/T
4-1: 81761870A/T
4-2: 9272951A/T
4-2: 17673300C/G
4-2: 17709304A/G
4-2: 17730189G/T
4-2: 27174776C/A
4-2: 30687825T/A
4-2: 31016844C/T
4-2: 32265161T/C
4-2: 40190519C/T
4-2: 41257170G/A
4-2: 48474813A/T
4-2: 51093580A/C
4-2: 51596740T/A
4-2: 52717454C/T
4-2: 56371492G/A
4-3: 4925640C/T
4-3: 9383087A/G
4-3: 9603540G/A
4-3: 11371361G/A
4-3: 18793380C/T
4-3: 20007275A/G
4-3: 20309838A/T
4-3: 21364593G/A
4-3: 26599303C/T
4-3: 28245014C/T
4-3: 29188428C/T
4-3: 30164923T/A
4-3: 30394503A/T
4-3: 32057601A/G
4-3: 32344999A/T
4-3: 35094329G/A
4-3: 35409750A/T
4-3: 36709093A/T
4-3: 39232209G/A
4-3: 39700804G/A
4-3: 39774507C/T
4-3: 43801940C/A
4-3: 44110784T/C
4-3: 44294813T/C
4-3: 44883647G/T
4-3: 45089871G/A
4-3: 45602740C/T
4-3: 47111722T/A
4-3: 49750223T/C
4-3: 50628138C/A
4-3: 55055947C/T
4-3: 55296677C/T
4-4: 3844208G/A
4-4: 5238981G/A
4-4: 5893163C/T
4-4: 7374175G/A
4-4: 15407346C/T
4-4: 15527633T/A
4-4: 20127266C/T
44: 22333281C/A
4-4: 23198882T/C
4-4: 24946004C/A
4-4: 27123194C/T
4-4: 27461340G/C
4-4: 32468993T/G
4-4: 32595583A/G
4-4: 41051587A/G
4-4: 41281369G/T
4-4: 43925275A/T
4-4: 47830865A/G
4-4: 51380820A/G
4-4: 51634934T/C
44: 51998786T/A
4-4: 60165534G/A
4-4: 66102358T/C
4-4: 67256102A/T
4-4: 69977979T/C
4-4: 70159631C/T
4-4: 70402558G/T
4-4: 80426946C/T
4-4: 80594362T/C
5-1: 20386888C/T
5-1: 20454590C/A
5-1: 20696195G/A
5-1: 20786144A/T
5-1: 23911441C/T
5-1: 24994982T/C
5-1: 29391790G/T
5-1: 30185436G/A
5-1: 30508074T/C
5-1: 40520428C/A
5-1: 40745846T/A
5-1: 42123007T/G
5-1: 42649907T/C
5-1: 42721663A/G
5-1: 48553095C/T
5-1: 49644419A/G
5-1: 54535808A/C
5-1: 56075031A/T
5-1: 61144458A/C
5-1: 61230013T/C
5-2: 7433039T/C
5-2: 7516325C/G
5-2: 9205888C/T
5-2: 10536428C/A
5-2: 22378081T/C
5-2: 24155840C/A
5-2: 24179099G/T
5-2: 24810461C/T
5-2: 25368529T/C
5-2: 30219694G/T
5-2: 30321389T/C
5-2: 30948170G/T
5-2: 35270508G/A
5-2: 36287996C/A
5-2: 36965207G/A
5-2: 41134455A/G
5-2: 41601740G/T
5-2: 43310343C/G
5-2: 44889846G/A
5-2: 45659963G/A
5-2: 45801436A/C
5-2: 52745514G/T
5-2: 52785599C/T
5-2: 56269894G/A
5-2: 56522059T/C
5-2: 58909436T/C
5-2: 60241802C/T
5-2: 67159841C/G
5-2: 67842265A/G
5-2: 69100778A/G
5-3: 1042304C/A
5-3: 1526116T/A
5-3: 7459483A/C
5-3: 10623532A/G
5-3: 11152091G/A
5-3: 14565946T/C
5-3: 16568950G/A
5-3: 18370194T/C
5-3: 21546518T/A
5-3: 21600270T/A
5-3: 22186160A/G
5-3: 31467598C/G
5-3: 31637896C/T
5-3: 34229334A/G
5-3: 37172371G/A
5-3: 37219944C/T
5-3: 40048220C/T
5-3: 41333442T/G
5-3: 44183561T/G
5-3: 45097620G/C
5-3: 45644480T/G
5-3: 46546603G/A
5-3: 48486772A/G
5-3: 50128584G/A
5-3: 50848329C/A
5-3: 51088542T/C
5-3: 53896114C/T
5-4: 4163304C/A
5-4: 6450436A/G
5-4: 10973323T/C
5-4: 20251267A/T
5-4: 21243323A/C
5-4: 23482426G/T
5-4: 24458563A/T
5-4: 24671948A/G
5-4: 25649627T/G
5-4: 28307072A/G
5-4: 28427508T/A
5-4: 28501905T/A
5-4: 28717375T/C
5-4: 30451345C/T
5-4: 31839976G/A
5-4: 35548648A/G
5-4: 43263841G/A
5-4: 43832440A/C
5-4: 43950850C/T
5-4: 45314941G/T
5-4: 46214683G/A
5-4: 46224063C/T
5-4: 46293839A/G
5-4: 47169835T/C
5-4: 48423152C/T
5-4: 48462877C/G
5-4: 48535924G/T
5-4: 52563150T/G
5-4: 54258739G/T
5-4: 54875287T/G
5-4: 55050774T/A
5-4: 56760003G/C
5-4: 58236747C/G
5-4: 58281713A/C
5-4: 60614484C/T
5-4: 61992385A/G
5-4: 62908772A/T
5-4: 69208948G/A
6-1: 2104467T/C
6-1: 4215903T/C
6-1: 6087900C/T
6-1: 11433181T/A
6-1: 12158561A/G
6-1: 14909476G/A
6-1: 15619643T/C
6-1: 15884993C/T
6-1: 16761454G/A
6-1: 17437967A/C
6-1: 17783570T/C
6-1: 17839382C/G
6-1: 18560083A/G
6-1: 19262474A/G
6-1: 31832144A/G
6-1: 32112499A/T
6-1: 35951056G/T
6-1: 49433805A/G
6-1: 50511068A/G
6-1: 52375070G/A
6-1: 54688238G/A
6-1: 60164135T/G
6-1: 65737222C/T
6-1: 66231744A/C
6-1: 66716014C/T
6-1: 67125944T/A
6-1: 69134108C/A
6-1: 69274840G/A
6-1: 71652645C/T
6-1: 72291256G/A
6-1: 73019922G/A
6-1: 73514501T/C
6-1: 73781356T/C
6-1: 74676491G/A
6-1: 74697990A/C
6-1: 75395713T/C
6-1: 80456478A/T
6-1: 83363675G/A
6-1: 83963027A/G
6-1: 84976820T/C
6-1: 85725185G/T
6-1: 88025561A/T
6-1: 90184492A/C
6-1: 90247896T/A
6-1: 92203793A/T
6-1: 93162903T/C
6-1: 93797280T/C
6-1: 94842258G/C
6-1: 95228397A/G
6-1: 95246918C/T
6-1: 96912292A/G
6-2: 783960T/C
6-2: 5977708C/T
6-2: 24336064C/A
6-2: 26114520C/G
6-2: 26608904G/A
6-2: 29552675G/A
6-2: 53269574C/T
6-2: 53846004A/C
6-2: 54468983C/T
6-2: 59714618C/T
6-2: 60731861T/C
6-2: 61885371T/A
6-2: 62901800C/T
6-2: 63146357T/A
6-2: 64418246T/A
6-2: 65397762G/C
6-2: 65542302G/A
6-2: 66484676G/A
6-2: 75633895A/G
6-2: 77092740C/T
6-3: 5309360T/A
6-3: 6137143T/C
6-3: 9543818A/C
6-3: 9608453C/A
6-3: 13010386A/T
6-3: 13256538G/C
6-3: 14158605G/A
6-3: 14282277A/C
6-3: 21384981G/C
6-3: 21692877T/C
6-3: 26688685C/G
6-3: 30140796T/A
6-3: 30396146G/A
6-3: 31018069C/T
6-3: 31092120C/G
6-3: 31622413C/A
6-3: 32911515G/A
6-3: 36929125G/A
6-3: 37427496G/A
6-3: 37472306A/T
6-3: 38578713G/T
6-3: 41263720C/A
6-3: 42921365G/A
6-3: 43880075G/A
6-3: 46977164C/T
6-3: 47218568T/C
6-3: 47346710G/A
6-3: 47562114G/A
6-3: 47822981G/A
6-3: 58812796G/A
6-3: 58997544A/G
6-3: 62021228A/G
6-3: 68300071A/G
6-3: 70084328C/T
6-3: 70462989T/A
6-3: 73349649A/G
6-3: 73545846T/A
6-3: 73637137G/A
6-3: 75268741C/G
6-3: 80134217T/C
6-3: 80309079G/T
6-3: 81325457C/T
6-3: 82142668T/C
6-3: 83273924G/A
6-3: 83665249C/A
6-3: 85257772G/A
6-3: 90354456C/T
6-3: 91723631C/T
6-3: 93334643C/T
6-3: 93385947T/A
6-4: 1657020C/G
6-4: 5451627A/G
6-4: 5732295A/G
6-4: 6303323T/A
6-4: 10604895A/C
6-4: 15211269C/T
6-4: 16236210A/C
6-4: 16376307C/T
6-4: 17005715T/C
6-4: 17943803A/G
6-4: 20162445A/C
6-4: 32355862A/C
6-4: 32596915A/G
6-4: 35359806C/T
6-4: 36257514C/A
6-4: 37090132A/T
6-4: 41585599C/G
6-4: 43539596C/T
6-4: 43652486C/T
6-4: 57207514A/G
6-4: 58304695C/T
6-4: 61632162T/C
6-4: 64718684T/C
6-4: 64955530G/A
6-4: 65143306G/A
6-4: 68427839C/A
6-4: 68735314G/A
6-4: 73192412C/T
6-4: 74388612G/A
6-4: 75462927C/T
6-4: 75570462C/T
6-4: 76028021T/A
6-4: 76582470T/C
6-4: 77857191G/C
6-4: 80266923G/A
6-4: 80316024G/A
6-4: 81877621T/G
6-4: 83592893A/C
7-1: 8320211A/G
7-1: 13912893A/G
7-1: 17203906A/T
7-1: 18825616C/T
7-1: 22468975A/C
7-1: 22695845T/C
7-1: 22856377T/A
7-1: 26329621G/A
7-1: 26384765G/A
7-1: 26480938A/T
7-1: 27488726T/C
7-1: 31812059C/T
7-1: 32773713G/A
7-1: 32843892C/A
7-1: 33091996C/T
7-1: 33601357A/C
7-1: 33926509C/T
7-1: 36905289A/G
7-1: 36997535C/T
7-1: 37202315A/G
7-1: 37532475G/A
7-1: 38098873A/G
7-1: 39607282T/C
7-1: 39813642T/A
7-1: 40280222C/A
7-1: 40351854G/A
7-1: 40512334A/T
7-1: 44980202A/T
7-1: 45088241G/A
7-1: 49383282A/G
7-1: 50057628A/G
7-1: 50464489A/G
7-1: 54437926A/G
7-1: 60065395A/G
7-1: 60213825G/A
7-1: 60294760G/A
7-1: 66476902C/T
7-1: 67190788T/C
7-1: 68742349C/T
7-1: 70983330C/A
7-1: 79932036C/A
7-2: 9633698G/A
7-2: 10154232T/C
7-2: 11633164C/T
7-2: 11889981A/G
7-2: 13540502T/A
7-2: 16531346T/C
7-2: 16720879G/A
7-2: 17144326G/A
7-2: 18022797A/G
7-2: 20639704T/C
7-2: 21767907A/T
7-2: 23744393T/G
7-2: 23849625A/T
7-2: 26058743A/C
7-2: 26068432G/A
7-2: 26680209G/A
7-2: 33326012A/G
7-2: 33959010T/C
7-2: 34210988A/C
7-2: 39532202C/T
7-2: 40543944T/C
7-2: 45545110C/T
7-2: 45691795A/C
7-2: 45808319T/C
7-2: 53010338C/G
7-2: 57259155A/G
7-2: 57885819C/T
7-2: 61473474G/A
7-2: 66851948C/T
7-2: 68682676T/C
7-2: 73311637C/T
7-2: 75350923C/T
7-2: 78904078A/T
7-2: 87010874A/T
7-3: 3729680G/A
7-3: 9001630A/T
7-3: 9179199G/T
7-3: 13001467A/G
7-3: 14063276C/T
7-3: 18528314C/T
7-3: 19760121A/T
7-3: 22265188T/C
7-3: 22616454C/T
7-3: 27912365C/T
7-3: 29607761C/A
7-3: 35510615G/A
7-3: 37866454T/C
7-3: 38770425G/C
7-3: 39570857A/G
7-3: 39761309C/T
7-3: 40838757A/G
7-3: 41433745G/T
7-3: 43222237T/G
7-3: 47375389A/G
7-3: 48220523A/T
7-3: 48488938T/G
7-3: 48520014T/C
7-3: 53482758T/A
7-3: 56041305T/C
7-3: 56453066A/T
7-3: 59205775C/T
7-3: 64232748C/T
7-3: 66212739G/T
7-3: 70385240T/G
7-3: 73950992T/A
7-3: 75692056A/G
7-3: 75877881T/A
7-3: 79013471C/T
7-3: 80308815T/C
7-3: 82861993C/T
7-4: 2302030T/C
7-4: 2847387A/G
7-4: 8540724G/A
7-4: 8770220T/A
7-4: 8903811A/C
7-4: 11184012T/A
7-4: 14351143C/T
7-4: 17213506C/T
7-4: 17285771G/A
7-4: 20433508T/C
7-4: 21448208T/C
7-4: 22535912T/C
7-4: 23551998G/A
7-4: 28269541G/A
7-4: 30840981T/C
7-4: 30892965T/G
7-4: 30952263T/A
7-4: 31100655A/G
7-4: 31357077A/G
7-4: 32268833C/T
7-4: 34889906G/A
7-4: 35964773T/A
7-4: 36814051G/A
7-4: 37527188G/C
7-4: 38263104G/A
7-4: 38749345A/G
7-4: 40621888A/G
7-4: 43849782T/A
7-4: 51794062C/A
7-4: 53096517C/G
8-1: 9954513A/T
8-1: 16541130T/C
8-1: 16629055C/A
8-1: 17349030C/T
8-1: 22723707G/A
8-1: 25018327G/A
8-1: 27302410A/C
8-1: 28151087T/A
8-1: 28774195T/A
8-1: 29682104A/C
8-1: 30440168C/T
8-1: 31923615G/A
8-1: 33553477A/C
8-1: 34218162T/A
8-1: 36576856A/G
8-1: 44231630G/A
8-1: 44295206T/A
8-1: 47475474C/T
8-1: 48814456T/A
8-1: 50963859A/C
8-1: 52918252T/G
8-1: 55421813T/C
8-1: 56651052T/G
8-1: 59286233C/T
8-1: 61017015T/G
8-1: 62198621C/T
8-1: 71682284G/T
8-1: 77212824G/A
8-1: 80574060A/C
8-1: 80666924C/T
8-1: 81379180G/A
8-1: 85363490G/A
8-1: 88554987G/A
8-2: 2510557C/T
8-2: 4041834T/A
8-2: 4232879C/T
8-2: 6431234A/G
8-2: 10306974C/T
8-2: 11746582T/C
8-2: 12055627G/A
8-2: 17170458T/G
8-2: 22164832C/T
8-2: 22995905A/T
8-2: 26534768A/C
8-2: 31235157C/T
8-2: 32000186T/A
8-2: 34575897A/T
8-2: 38787886C/T
8-2: 38909253T/C
8-2: 40835554G/T
8-2: 41479347T/C
8-2: 42968267A/G
8-2: 47504535T/C
8-2: 48075172C/T
8-2: 48505072T/C
8-2: 48717810A/C
8-2: 49003665A/T
8-2: 50024089G/A
8-2: 51375204G/A
8-2: 52269420C/A
8-2: 54072710C/A
8-2: 55776868T/A
8-2: 70745916A/G
8-3: 1912936A/G
8-3: 2453305T/C
8-3: 6933071C/T
8-3: 11559831C/G
8-3: 17998748G/A
8-3: 18335531G/A
8-3: 21715571T/A
8-3: 26017615A/C
8-3: 33888410C/T
8-3: 37610274C/T
8-3: 42111805C/T
8-3: 44244432C/T
8-3: 50470807T/A
8-3: 51626021C/T
8-3: 52654499C/T
8-3: 54290508A/G
8-3: 55138906A/G
8-3: 60988062T/A
8-3: 62958659C/T
8-3: 65451881A/G
8-4: 6770025C/A
8-4: 10746985A/G
8-4: 13150129T/G
8-4: 17200826G/T
8-4: 17541042G/T
8-4: 19117141T/C
8-4: 25149710T/C
8-4: 27617446C/A
8-4: 27650134T/A
8-4: 30700906T/C
8-4: 32634989A/T
8-4: 33100007T/G
8-4: 33122868A/T
8-4: 37783181C/T
8-4: 43470325T/C
8-4: 43753260G/A
8-4: 44623676T/C
8-4: 52635712A/G
8-4: 52714851T/C

In some embodiments of the present disclosure, Alt is “.”, indicating various mutation possibilities.

In some embodiments of the present disclosure, when ref is A, Alt is “.”, indicating the mutation can be T, G, C, or N; when ref is T, Alt is “.”, indicating the mutation can be A, G, C, or N; when ref is G, Alt is “.”, indicating the mutation can be A, T, C, or N; and when ref is C, Alt is “.”, indicating the mutation can be A, T, G, or N, wherein N is deletion.

In some embodiments of the present disclosure, the “−” in the table can be “_”.

In some embodiments of the present disclosure, the position information of the SNP molecular markers are represented in the form of chromosome numbering: physical position.

In some embodiments of the present disclosure, the 61351 SNP molecular markers are as displayed in SNP00001-61351; and the specific locus information of SNP00001-61351 are sorted from top to bottom and from left to right in Table 1.

According to the second aspect of the present disclosure, a primer set and/or liquid-phase probe for detecting the above SNP molecular marker array of alfalfa is provided.

In some embodiments of the present disclosure, the liquid-phase probe is designed and synthesized based on the position of the SNP locus and sequences that flank the SNP locus by means of targeted capture sequencing technology.

In some embodiments of the present disclosure, the liquid-phase probe has a length of about 100 bp and a GC content between 20% and 80%.

According to the third aspect of the present disclosure, a genome-wide liquid-phase chip of alfalfa is provided, wherein the genome-wide liquid-phase chip of alfalfa comprises a primer set and/or liquid-phase probe for detecting the above SNP molecular marker array of alfalfa.

The genome-wide liquid-phase chip of alfalfa of the present disclosure is based on the targeted capture and can be applied to efficient and precise genotyping assay on large-scale alfalfa populations at a low cost. (1) In terms of performance, all the SNP loci of this chip are derived from genome-wide resequencing data, and compared to widely used reduced-representation genome sequencing technologies (such as RAD-seq and GBS (Genotyping-by-Sequencing)), these loci have a broader coverage across the whole genome of alfalfa, a better representativeness and a higher polymorphism, and are advantageous in terms of both detection accuracy and density. (2) In terms of economic costs, compared with currently widely used genome-wide resequencing methods, which mostly cost 1000-1200 yuan per sample for the genotyping assay of alfalfa, and imported sequencers (such as Illumina and Thermo Fisher), which lead to higher prices, the technique using this SNP array results in a low detection cost of 200 yuan per sample or less, representing a greatly reduced detection cost; moreover, the chip has flexible loci, allowing for the addition of new functional marker loci at any time in the future, demonstrating strong potential for application and promotion. (3) This chip comprises 1448 trait-associated loci, which can be directly used for the identification, evaluation, development, and utilization of important economic traits such as yield, quality, and stress resistance of alfalfa germplasm materials, indicating that the practicability is high.

According to the fourth aspect of the present disclosure, a kit is provided, wherein the kit comprises a primer set and/or liquid-phase probe for detecting the above SNP molecular markers of alfalfa.

According to the fifth aspect of the present disclosure, the use of the above SNP molecular marker array of alfalfa, kit, or genome-wide liquid-phase chip of alfalfa is provided.

In some embodiments of the present disclosure, the use is the genotyping of alfalfa.

In some embodiments of the present disclosure, the use is the genome-wide association analysis of alfalfa.

In some embodiments of the present disclosure, the use is the cluster analysis or genetic relationship identification of alfalfa.

In some embodiments of the present disclosure, the use is the evaluation of genetic diversity in alfalfa.

In some embodiments of the present disclosure, the use is the breeding or assisted breeding of alfalfa.

In some embodiments of the present disclosure, the breeding or assisted breeding comprises at least one of major effect genes selection, molecular marker-assisted breeding for directional improvement, genome-wide selection breeding, verification of alfalfa variety authenticity, genetic map construction, gene mapping, and evaluation of genetic diversity in germplasm resources.

According to the sixth aspect of the present disclosure, a method for breeding alfalfa is provided, comprising the following steps: using at least one of the above SNP molecular markers, kit, or genome-wide liquid-phase chip to detect DNA in alfalfa, and selecting the appropriate alfalfa germplasm materials for subsequent breeding.

In some embodiments of the present disclosure, the detection is based on liquid-phase probe capture and sequencing typing technology.

The present disclosure has at least the following beneficial effects: the SNP molecular markers of the present disclosure have good locus-specificity, excellent versatility, and high polymorphism and can accurately and effectively distinguish alfalfa germplasm resources of different origins with different characteristics. A genome-wide liquid-phase gene chip of alfalfa prepared using the SNP molecular markers of the present disclosure can be used for genetic structure analysis in alfalfa germplasm resource populations, assessment of genetic diversity, genetic map construction, functional gene mapping, genome-wide association analysis, etc., providing an essential tool and technical support for molecular design breeding of alfalfa and verification of variety authenticity. The present disclosure is significant for the high-quality advancement of the alfalfa seed industry.

### BRIEF DESCRIPTION OF THE DRAWINGS

The present disclosure will be further described below with reference to the accompanying drawings and examples, in which:

FIG. 1 is an MAF distribution histogram of Example 1 of the present disclosure;

FIG. 2 is a chromosomal distribution map of alfalfa 60K cGPS liquid-phase chip loci in Example 1 of the present disclosure;

FIG. 3 is a schematic diagram of the detection workflow by a cGPS liquid-phase chip in Example 2 of the present disclosure;

FIG. 4 is a cluster analysis diagram of alfalfa in Example 4 of the present disclosure.

### DETAILED DESCRIPTION

The concept and generated technical effects of the present disclosure will be described below clearly and completely combined with the examples, so as to fully understand the objects, features, and effects of the present disclosure. Apparently, the described examples are only a part of the embodiments of the present invention, not all examples. Based on the examples of the present disclosure, other examples obtained by those skilled in the art without involving creative labor shall fall within the scope of protection of the present disclosure. Where specific conditions are not specified in the examples, standard conditions or those recommended by the manufacturer shall apply. The reagents or instruments used without specifying the manufacturer are all commercially available conventional products.

### Example 1

This example provided a SNP molecular marker array of alfalfa comprising 61351 SNP loci, wherein the physical positions of the 61351 SNP loci were determined by sequence alignment based on the reference of genome alfalfa (ZM-4 alfalfa genome, www.figshare.com/s/fb4ba8e0b871007a9e6c). The 61351 SNP molecular markers are as shown above (see the table 1 for detailed information). The screening process therefor was specifically as follows:

1. Acquisition of a Set of Candidate SNP Loci Derived from Genome-Wide Resequencing Data

In order to obtain genome-wide loci with rich genetic variation, we collected resequencing data of 358 alfalfa core germplasm resources worldwide, published by the National Genome Data Center (www.ngdc.cncb.ac.cn/). Additionally, we gathered 380 alfalfa germplasm materials from various countries such as China, the United States, Russia, Spain and others, for genome-wide resequencing. Libraries were sequenced using MGI's DNBSEQ-T7 high-throughput sequencing platform. SNP calling was performed following a workflow involving: first filtering with fastp (v0.20.0); then aligning against the alfalfa reference genome (ZM-4 alfalfa genome, www.figshare.com/s/fb4ba8e0b871007a9e6c) by means of Burrows-Wheeler Aligner (0.7.12-r1039); Subsequently, Picard tools version 1.107 (www.psc.edu/index.php/user-resources/software/picard) sorted the SAM files and converted them to bam format, and removed PCR duplicates; SNP detection and filtering were performed using GATK software, and the following filtering criteria were applied based on the genomic data: QD<2.0∥FS>60.0∥MQ<35.0∥MQRankSum<−12.5∥Read PosRankSum<−8.0∥DP>6950. After filtering, a .vcf file containing SNP variation information for all samples was obtained, comprising a total of 4,052,804 SNP loci.

### 2. Screening for Loci

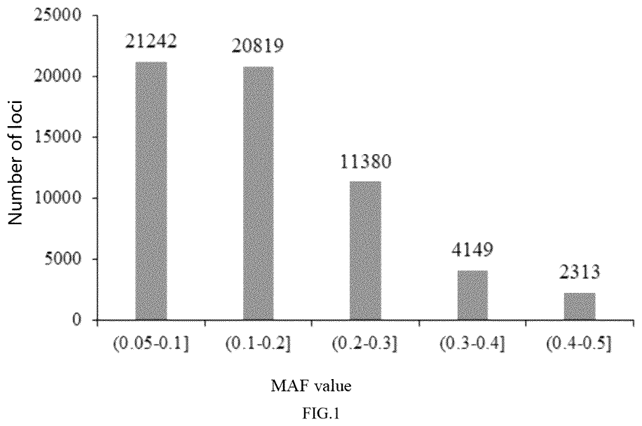

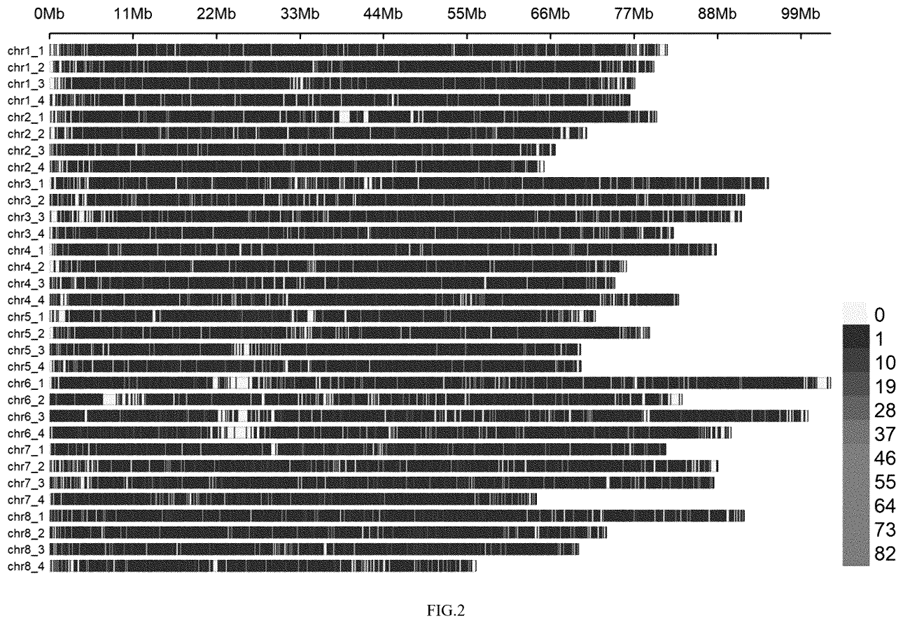

Quality indicators were calculated for all loci and screened 1739982 SNP loci based on parameters such as Maf≥0.05, detection rate ≥85%, heterozygosity ≤0.5, and Depth ≥5. These loci will be used for probe design. The principle of probe design involves designing probes for all target sites within a 100 bp flanking region on either side. Probes are typically 100 bp in length, with a GC content ranging from 20% to 80%. Based on the probe design results, probes that could not be uniquely mapped to the genome or contained repetitive sequences in the flanking regions were removed. Following the principle of uniform distribution across chromosomes, a total of 59903 high-polymorphism SNP loci were ultimately obtained, as shown in FIG. 1. Functional genes previously reported to be associated with important economic traits of alfalfa, such as growth and development, quality, and stress resistance, were collected. A total of 1448 SNP loci were screened from these genes as candidate loci, including 295 loci associated with growth and development traits, 1129 loci associated with resistance (including biotic and abiotic stress resistance), 11 loci linked to quality traits, and 12 loci of unknown functions. Ultimately, 61351 candidate SNP loci were obtained. The information of the loci is presented in Table 1 with an average spacing of 41 Kb. Chromosomal distribution map is as shown in FIG. 2.

### Example 2

This example provided an alfalfa 60K genome-wide liquid-phase chip.

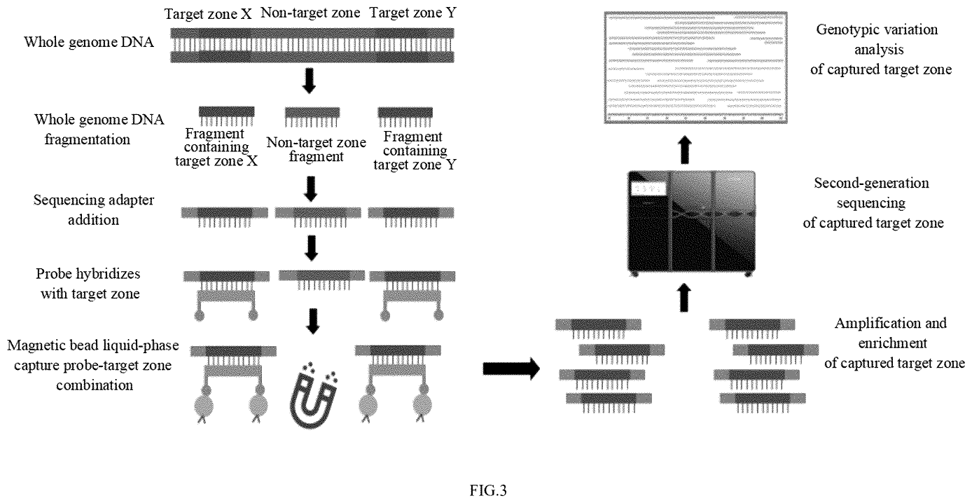

Based on the upstream and downstream sequences of the 61351 SNP polymorphic loci in alfalfa, and following the principle of probe design in Example 1, liquid-phase capture probes were synthesized by Higentec Co., Ltd. By means of a technology of genotyping by pinpoint sequencing of captured target region genomic sequences (cGPS) in liquid phase, a genome-wide liquid-phase chip system of alfalfa was formed. cGPS employs an optimized thermodynamic stability algorithm model to design probes for genomic sequences across different target regions. The synthesized specific probes were used to perform liquid-phase hybridization capture enrichment for a plurality of distinct target sequences located at different genomic positions. Subsequently, the captured and enriched target genomic sequences were used for library construction and high-throughput sequencing, thereby obtaining genotypes of all SNP/InDel loci within the target regions. A schematic diagram of cGPS-based liquid-phase array detection workflow is as shown in FIG. 3.

### Example 3: Use of Alfalfa 60K Genome-Wide Liquid-Phase Array in Detection of Alfalfa DNA Samples

1. Extraction and Detection of Alfalfa gDNA

25 alfalfa germplasm materials were selected as validation samples for the developed alfalfa 60K genome-wide liquid-phase chip system, 25 fresh leaf samples of alfalfa materials were respectively collected, and gDNA was extracted from the tissues using a magnetic bead-based method. The integrity and purity of the gDNA were analyzed by a 1% agarose gel electrophoresis method, and the concentration was accurately quantified by means of Qubit.

2. cGPS Experiment Process
(1) A fixed amount of 200 ng of qualified gDNA was taken, the DNA was digested into fragments of 100-500 bp in size by means of a digestion enzyme reagent, and then, Taq enzyme was added for end repair;
(2) Adapter fragments were ligated to both ends of the DNA using T4 ligase, and the ligated products were purified by means of fragment selection magnetic beads, followed by library amplification, thus completing the library construction;
(3) The qualified libraries, along with a blocking reagent, RNase Inhibitor (Rnase Block), and 60K liquid-phase chip probes, were placed on a PCR instrument for a hybridization reaction, wherein hybridization incubation was carried out overnight at 55° C. (16-24 h);
(4) The hybridized products were captured by means of streptavidin, the captured libraries were amplified and enriched, and PE150 sequencing was performed on a MGISEQ-2000 sequencing platform (MGI Tech Co., Ltd.);
(5) The raw data obtained after high-throughput sequencing were subjected to processing such as quality control filtering, and the reads with adapter contamination and low-quality reads were removed using fastp software. After alignment to the target genome by means of BWA software, the sequencing data were subjected to variant site analysis using GATK software to obtain the genotyping results of the target loci. A schematic diagram of the workflow is as shown in FIG. 3.

TABLE 2

| | Number | | Detection | |
|---|---|---|---|---|
| Sample Name | of loci | Heterozygosity | rate | Origin |
| 10890 | 61351 | 12.78% | 91.16% | Russia |
| 14139 | 61351 | 12.88% | 90.78% | Russia |
| 213MG | 61351 | 17.87% | 96.04% | China |
| 4020 | 61351 | 13.31% | 91.68% | Canada |
| 4020-re | 61351 | 13.27% | 91.24% | Canada |
| 8659 | 61351 | 12.73% | 91.21% | China |
| BG | 61351 | 16.90% | 95.88% | Oregon |
| JN601 | 61351 | 12.66% | 91.90% | Oregon |
| KH | 61351 | 18.66% | 96.57% | Oregon |
| P01-A01 | 61351 | 13.00% | 91.90% | Spain |
| P01-C02 | 61351 | 12.85% | 91.61% | Spain |
| P01-C03 | 61351 | 13.37% | 91.57% | US |
| P01-C05 | 61351 | 17.33% | 96.75% | Russia |
| P01-C06 | 61351 | 13.17% | 91.59% | Russia |
| P01-C07 | 61351 | 12.89% | 91.76% | Russia |
| P01-C08 | 61351 | 13.51% | 91.84% | Bulgaria |
| P01-C09 | 61351 | 13.23% | 92.02% | Spain |
| P01-C10 | 61351 | 12.95% | 92.08% | Tajikistan |
| P01-C10-re | 61351 | 13.08% | 92.01% | Tajikistan |
| P02-A05 | 61351 | 13.58% | 93.28% | Canada |
| P02-B05 | 61351 | 12.58% | 93.04% | France |
| SBD | 61351 | 11.94% | 90.15% | Canada |
| WL319 | 61331 | 13.44% | 91.66% | Oregon |
| WL349 | 61351 | 16.43% | 96.00% | Oregon |
| WL358 | 61351 | 12.15% | 90.35% | US |
| YDA | 61351 | 11.93% | 90.00% | US |
| ZM-4 | 61351 | 12.23% | 91.16% | China |
| ZM-4-re | 61351 | 12.22% | 90.81% | China |
| Avarage | 61351 | 13.48% | 92.11% | — |

TABLE 3

| | | Number of | Number of | Genotype |
|---|---|---|---|---|
| Sample | Sample | consistent | inconsistent | consistency |
| name 1 | name 2 | loci | loci | rate |
| P01-C10 | P01-C10-re | 53740 | 1647 | 97.03% |
| ZM-4 | ZM-4-re | 52910 | 1561 | 97.13% |
| 4020 | 4020-re | 53375 | 1604 | 97.08% |

| | | | |
|---|---|---|---|
| Avarage | — | — | 97.08% |

### 3. Assessment of Genotyping Effect of Alfalfa 60K Liquid-Phase Chip

The 60K liquid-phase chip obtained in Example 1 was used for a genotyping assay on 25 alfalfa materials (see Example 2 for the specific operation method). A total of 28 pieces of data were generated (including 3 replicate samples). The results are as shown in Table 2 and Table 3. As shown in Table 2, the loci detection rate across the 28 samples was between 90.00% and 96.75%, with an average detection rate of 92.11%. As shown in Table 3, the results of genotypic comparison of the three replicate samples showed a consistency rate between 97.03% and 97.13%, with an average consistency rate of 97.08%.

### Example 4: Analysis of Alfalfa Population Structure by Alfalfa 60K Genome-Wide Liquid-Phase Array

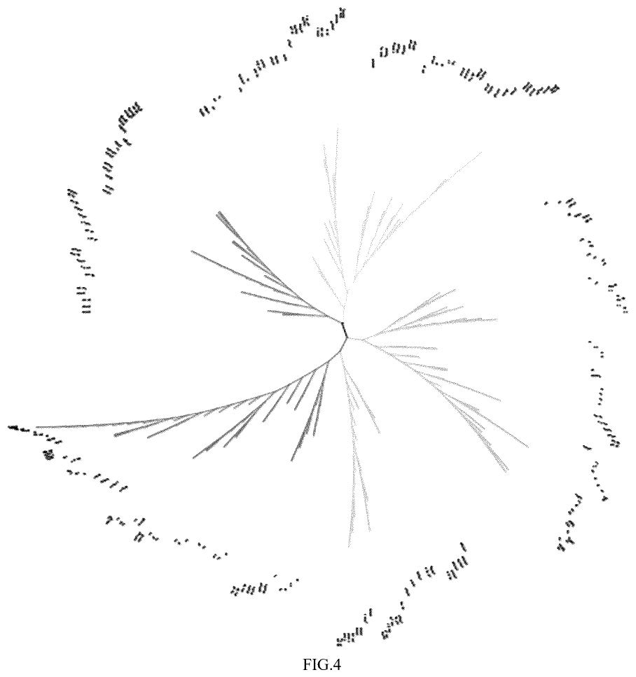

457 alfalfa germplasm samples worldwide (of known origins) were genotyped using the alfalfa 60K genome-wide liquid-phase array prepared in Example 2. A genetic distance matrix was calculated based on IBS (Identity By State) method using Plink software, followed by cluster analysis to construct a phylogenetic tree, so that the genetic relationships, evolutionary relationships, and population structure among the various materials could be determined. The result was shown in FIG. 4, from which it can be seen that the alfalfa germplasm samples of the same geographical origin or from the same continent were clustered into one cluster, indicating that there is a significant genetic variation among alfalfa germplasm from different regions. This liquid-phase array can well analyze genetic diversity and population structure.

The examples of the present disclosure have been described in detail above. However, the present disclosure is not limited to the above examples. Various changes can also be made within the knowledge of those of ordinary skill in the pertinent technical field without departing from the gist of the present disclosure. In addition, the examples of the present disclosure and the features in the examples can be combined with each other without conflict.

1. A SNP molecular marker array of alfalfa, wherein the SNP molecular marker array is composed of 61351 SNP molecular markers; physical positions of the 61351 SNP molecular markers are determined by sequence alignment based on a reference genome ZM-4 alfalfa genome of alfalfa, and locus information is as specifically shown in Table 1 in the description.

2. A primer set and/or liquid-phase probe for detecting the SNP molecular marker array of alfalfa according to claim 1.

3. A genome-wide liquid-phase chip of alfalfa, wherein the genome-wide liquid-phase chip of alfalfa comprises the primer set and/or liquid-phase probe according to claim 2.

4. A kit comprising the primer set and/or liquid-phase probe according to claim 2.

5. (canceled)

6. (canceled)

7. (canceled)

8. (canceled)

9. A method for breeding of alfalfa, wherein the method comprises the step of using the SNP molecular marker array of alfalfa according to claim 1 to detect DNA in a sample to be detected.

10. A method for breeding alfalfa, wherein the method comprises the steps of using the SNP molecular marker array of alfalfa according to claim 1 to detect DNA in a sample to be detected, and selecting appropriate alfalfa germplasm materials for subsequent breeding.

11. A method for breeding alfalfa, wherein the method comprises the steps of using the primer set and/or liquid-phase probe according to claim 2 to detect DNA in a sample to be detected, and selecting alfalfa germplasm materials for subsequent breeding.

12. A method for breeding alfalfa, wherein the method comprises the steps of using the genome-wide liquid-phase chip of alfalfa according to claim 3 to detect DNA in a sample to be detected, and selecting alfalfa germplasm materials for subsequent breeding.

13. A method for breeding alfalfa, wherein the method comprises the steps of using the kit according to claim 4 to detect DNA in a sample to be detected, and selecting alfalfa germplasm materials for subsequent breeding.

14. The method according to claim 9, wherein the breeding or assisted breeding comprises at least one of major effect gene selection, molecular marker-assisted breeding for directional improvement, genome-wide selection breeding, alfalfa variety identification, gene mapping, and evaluation of genetic diversity in germplasm resources.

15. A method for breeding or assisted breeding of alfalfa, wherein the method comprises the step of using the primer set and/or liquid-phase probe according to claim 2 to detect DNA in a sample to be detected.

16. A method for breeding or assisted breeding of alfalfa, wherein the method comprises the step of using the genome-wide liquid-phase chip of alfalfa according to claim 3 to detect DNA in a sample to be detected.

17. A method for breeding or assisted breeding of alfalfa, wherein the method comprises the step of using the kit according to claim 4 to detect DNA in a sample to be detected.

18. A method used in the genotyping of alfalfa, the genome-wide association analysis, the cluster analysis or the genetic diversity identification of alfalfa, wherein the method comprises the step of using the SNP molecular marker array of alfalfa according to claim 1 to detect DNA in a sample to be detected.

19. A method used in the genotyping of alfalfa, the genome-wide association analysis, the cluster analysis or the genetic diversity identification of alfalfa, wherein the method comprises the step of using the primer set and/or liquid-phase probe according to claim 2 to detect DNA in a sample to be detected.

20. A method used in the genotyping of alfalfa, the genome-wide association analysis, the cluster analysis or genetic relationship identification of alfalfa, wherein the method comprises the step of using the genome-wide liquid-phase probe of alfalfa according to claim 3 to detect DNA in a sample to be detected.

21. A method used in the genotyping of alfalfa, the genome-wide association analysis, the cluster analysis or the genetic diversity identification of alfalfa, wherein the method comprises the step of using the kit according to claim 4 to detect DNA in a sample to be detected.
